(19) United States
(12) Patent Application Publication
(10) Pub. No.: US 2025/0025478 A1
(43) Pub. Date: Jan. 23, 2025

(54) METHODS OF TARGETING RNF213 SIGNALING PATHWAY FOR TREATING DISEASES

(71) Applicant: New York University, New York, NY (US)

(72) Inventors: Benjamin NEEL, New York, NY (US); Abhishek Bhardwaj, New York, NY (US)

(73) Assignee: New York University, New York, NY (US)

(21) Appl. No.: 18/763,300

(22) Filed: Jul. 3, 2024

Related U.S. Application Data
(60) Provisional application No. 63/524,807, filed on Jul. 3, 2023.

(51) Int. Cl. A61K 31/575; A61K 31/4439; A61K 31/506; A61K 45/06; A61P 35/00; C12N 15/113

(52) U.S. Cl. CPC A61K 31/575; A61K 31/4439; A61K 31/506; A61K 45/06; A61P 35/00; C12N 15/1137; C12N 2310/14; C12N 2310/531

(57) ABSTRACT

This application provides methods of inhibiting an inflammation- or hypoxia-induced death of a cell involving inhibiting an RNF213 signaling pathway. The application further provides methods of preventing or treating various diseases and disorders in a subject involving administering to the subject an inhibitor of expression of RNF213 protein or an inhibitor of one or more functions of the RNF213 protein. The application still further provides methods for enhancing the effectiveness of a cancer treatment in a subject where the cancer involves a hypoxic tumor microenvironment involving co-administering the cancer treatment with an inhibitor or activator of different functions of the RNF213 protein. The application also provides methods of eliminating drug-resistant tumor cells by co-administering an inhibitor of the expression of RNF213 protein or by inhibiting or activating specific functions of the RNF213 protein.

### CROSS-REFERENCE TO RELATED APPLICATIONS

This patent application claims priority to U.S. Provisional Application No. 63/524,807, filed on Jul. 3, 2023, the disclosure of which is herein incorporated by reference in its entirety.

### STATEMENT REGARDING FEDERALLY SPONSORED RESEARCH

This invention was made with government support under P30 CA016087, R01 CA264933, and R01 CA049152 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jun. 27, 2024, is named 243735_000376_SL.xml and is 4,502,680 bytes in size.

### FIELD OF THE DISCLOSURE

This application relates to methods of inhibiting an inflammation- or hypoxia-induced death of a cell involving inhibiting an RNF213 signaling pathway. The application further relates to methods of preventing or treating various diseases and disorders (e.g., vascular occlusive events or disorders, autoimmune or inflammatory diseases, diseases associated with lipotoxicity, liver damage, cancer) in a subject involving administering to the subject an inhibitor of expression of RNF213 protein or an inhibitor of one or more functions of the RNF213 protein. The application still further relates to methods for enhancing the effectiveness of a cancer treatment in a subject where the cancer involves a hypoxic tumor microenvironment involving co-administering the cancer treatment with an inhibitor or activator of different functions of the RNF213 protein. The application also relates to methods of eliminating drug-resistant tumor cells (a.k.a. drug-tolerant persister cells, DTPs) by co-administering an inhibitor of the expression of RNF213 protein or by inhibiting or activating specific functions of the RNF213 protein.

### BACKGROUND

Heart attack and stroke are the leading causes of death globally, often necessitating surgical intervention in addition to medications such as statins, thiazide diuretics, beta-blockers, and alpha-blockers to prevent further harm. Most drugs do not specifically target these conditions. Moyamoya disease (MMD) is a rare syndrome causing precocious strokes, often in teenagers and young adults, that are characterized by steno-occlusion of the carotid artery, a critical supplier of blood to the brain. MMD is also associated with more common types of steno-occlusive diseases and lipotoxicity. Currently, there is no effective medical treatment for MMD aside from complex surgical interventions. Single nucleotide polymorphisms (SNPs) in RNF213 are known to be associated with MMD. RNF213 is a giant (˜600 kDa) AAA-ATPase/ubiquitin ligase, which oligomerizes via its ATPase domains and has two potential E3 ubiquitin ligase domains (RING, RZ). RNF213 has been shown to protect the host against various pathogens through its oligomerized form and its RZ domain activity. Prior work has established that PTP1B negatively regulates the activity of RNF213 ubiquitin ligase activity and that loss of PTP1B regulation of RNF213 led to cell death in hypoxia. In particular PTP1B deficiency or inhibition can increase the death of HER2+ breast cancer cells maintained at ≤1% O₂. Most of the highly penetrant MMD SNPs reside within the RING domain of RNF213.

In HER2+ breast cancer, a common problem with current established HER2+ breast cancer treatment regimens is the emergence of drug-tolerant persisters (DTPs), which can seed tumor recurrence.

### SUMMARY OF THE INVENTION

As specified in the Background section above, there is a great need in the art for development of new therapeutic strategies for Moyamoya disease (MMD), as well as for patients with more conventional occlusive events (e.g., stroke, myocardial infarction, arterial thrombosis), diseases of lipotoxicity (e.g., hepatic steatosis), and cancer (e.g., HER2+ breast cancer). The present application addresses these and other needs.

In one aspect, provided herein is a method of inhibiting inflammatory cell death in a subject in need thereof, comprising inhibiting the RZ domain of RNF213 protein in the cells of the subject.

In another aspect, provided herein is a method of inhibiting inflammatory cell death in a subject in need thereof, comprising inhibiting a functional ATPase domain of RNF213 protein in the cells of the subject.

In another aspect, provided herein is a method of inhibiting inflammatory cell death in a subject in need thereof, comprising inhibiting RNF213 protein expression or degrading RNF213 protein in the cells of the subject.

In another aspect, provided herein is a method of inhibiting inflammatory cell death in a subject in need thereof, comprising activating the RING domain of RNF213 protein in the cells of the subject.

In another aspect, provided herein is a method of inhibiting inflammatory cell death in a subject in need thereof, comprising inhibiting oligomerization of RNF213 protein in the cells of the subject.

In another aspect, provided herein is a method of inhibiting inflammatory cell death in a subject in need thereof, comprising inhibiting an RNF213 activator in the cells of the subject.

In some embodiments of any of the above-described methods, the inflammatory cell death is pyroptotic cell death.

In another aspect, provided herein is a method of inhibiting hypoxia-induced cell death in a subject in need thereof, comprising inhibiting the RZ domain of RNF213 protein in the cells of the subject.

In another aspect, provided herein is a method of inhibiting hypoxia-induced cell death in a subject in need thereof, comprising inhibiting a functional ATPase domain of RNF213 protein in the cells of the subject.

In another aspect, provided herein is a method of inhibiting hypoxia-induced cell death in a subject in need thereof, comprising inhibiting RNF213 protein expression or degrading RNF213 protein in the cells of the subject.

In another aspect, provided herein is a method of inhibiting hypoxia-induced cell death in a subject in need thereof, comprising activating the RING domain of RNF213 protein in the cells of the subject.

In another aspect, provided herein is a method of inhibiting hypoxia-induced cell death in a subject in need thereof, comprising inhibiting oligomerization of RNF213 protein in the cells of the subject.

In another aspect, provided herein is a method of inhibiting hypoxia-induced cell death in a subject in need thereof, comprising inhibiting an RNF213 activator in the cells of the subject.

In another aspect, provided herein is a method of preventing or treating a vascular occlusive event or disorder in a subject in need thereof, comprising inhibiting the RZ domain of RNF213 protein in the cells of the subject.

In another aspect, provided herein is a method of preventing or treating a vascular occlusive event or disorder in a subject in need thereof, comprising inhibiting a functional ATPase domain of RNF213 protein in the cells of the subject.

In another aspect, provided herein is a method of preventing or treating a vascular occlusive event or disorder in a subject in need thereof, comprising inhibiting RNF213 protein expression or degrading RNF213 protein in the cells of the subject.

In another aspect, provided herein is a method of preventing or treating a vascular occlusive event or disorder in a subject in need thereof, comprising activating the RING domain of RNF213 protein in the cells of the subject.

In another aspect, provided herein is a method of preventing or treating a vascular occlusive event or disorder in a subject in need thereof, comprising inhibiting oligomerization of RNF213 protein in the cells of the subject.

In another aspect, provided herein is a method of preventing or treating a vascular occlusive event or disorder in a subject in need thereof, comprising inhibiting an RNF213 activator in the cells of the subject.

In some embodiments of any of the above-described methods, the vascular occlusive event or disorder is a stroke, myocardial infarction, arterial thrombosis, atherosclerosis, peripheral arterial disease, renal vascular disease, Moyamoya disease (MMD), or Moyamoya syndrome.

In another aspect, provided herein is a method of preventing or treating an autoimmune or inflammatory disease in a subject in need thereof, comprising inhibiting the RZ domain of RNF213 protein in the cells of the subject.

In another aspect, provided herein is a method of preventing or treating an autoimmune or inflammatory disease in a subject in need thereof, comprising inhibiting a functional ATPase domain of RNF213 protein in the cells of the subject.

In another aspect, provided herein is a method of preventing or treating an autoimmune or inflammatory disease in a subject in need thereof, comprising inhibiting RNF213 protein expression or degrading RNF213 protein in the cells of the subject.

In another aspect, provided herein is a method of preventing or treating an autoimmune or inflammatory disease in a subject in need thereof, comprising activating the RING domain of RNF213 protein in the cells of the subject.

In another aspect, provided herein is a method of preventing or treating an autoimmune or inflammatory disease in a subject in need thereof, comprising inhibiting oligomerization of RNF213 protein in the cells of the subject.

In another aspect, provided herein is a method of preventing or treating an autoimmune or inflammatory disease in a subject in need thereof, comprising inhibiting an RNF213 activator in the cells of the subject.

In some embodiments of any of the above-described methods, the autoimmune or inflammatory disease is Graves' disease, systemic lupus erythematosus, Sjögren's syndrome, multiple sclerosis, or rheumatoid arthritis.

In another aspect, provided herein is a method of preventing or treating a disease associated with lipotoxicity in a subject in need thereof, comprising inhibiting the RZ domain of RNF213 protein in the cells of the subject.

In another aspect, provided herein is a method of preventing or treating a disease associated with lipotoxicity in a subject in need thereof, comprising inhibiting a functional ATPase domain of RNF213 protein in the cells of the subject.

In another aspect, provided herein is a method of preventing or treating a disease associated with lipotoxicity in a subject in need thereof, comprising activating the RING domain of RNF213 protein in the cells of the subject.

In another aspect, provided herein is a method of preventing or treating a disease associated with lipotoxicity in a subject in need thereof, comprising inhibiting RNF213 protein expression or degrading RNF213 protein in the cells of the subject.

In another aspect, provided herein is a method of preventing or treating a disease associated with lipotoxicity in a subject in need thereof, comprising inhibiting oligomerization of RNF213 protein in the cells of the subject.

In another aspect, provided herein is a method of preventing or treating a disease associated with lipotoxicity in a subject in need thereof, comprising inhibiting an RNF213 activator in the cells of the subject.

In some embodiments of any of the above-described methods, the disease associated with lipotoxicity is hepatic steatosis, non-alcoholic steatohepatitis (NASH), non-alcoholic fatty liver disease (NAFLD), primary lipodystrophy, or secondary lipodystrophy.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising inhibiting the RZ domain of RNF213 protein in the cells of the subject.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising inhibiting a functional ATPase domain of RNF213 protein in the cells of the subject.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising activating the RING domain of RNF213 protein in the cells of the subject.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising inhibiting RNF213 protein expression or degrading RNF213 protein in the cells of the subject.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising inhibiting oligomerization of RNF213 protein in the cells of the subject.

In another aspect, provided herein is a method of preventing or limiting liver damage in a subject having hepatic steatosis, non-alcoholic steatohepatitis (NASH), non-alcoholic fatty liver disease (NAFLD), primary lipodystrophy, or secondary lipodystrophy, the method comprising inhibiting an RNF213 activator in the cells of the subject.

In another aspect, provided herein is a method of preventing the generation of drug-tolerant persister cancer cells (DTPs) in a subject in need thereof who has been treated with a tyrosine kinase inhibitor (TKI), comprising inhibiting a functional ATPase domain of RNF213 protein in the cells of the subject.

In another aspect, provided herein is a method of preventing the generation of drug-tolerant persister cancer cells (DTPs) in a subject in need thereof who has been treated with a tyrosine kinase inhibitor (TKI), comprising inhibiting RNF213 protein expression or degrading RNF213 protein in the cells of the subject.

In another aspect, provided herein is a method of preventing the generation of drug-tolerant persister cancer cells (DTPs) in a subject in need thereof who has been treated with a tyrosine kinase inhibitor (TKI), comprising inhibiting the RING domain of RNF213 protein in the cells of the subject.

In another aspect, provided herein is a method of preventing the generation of drug-tolerant persister cancer cells (DTPs) in a subject in need thereof who has been treated with a tyrosine kinase inhibitor (TKI), comprising inhibiting oligomerization of RNF213 protein in the cells of the subject.

In another aspect, provided herein is a method of preventing the generation of drug-tolerant persister cancer cells (DTPs) in a subject in need thereof who has been treated with a tyrosine kinase inhibitor (TKI), comprising inhibiting an RNF213 activator in the cells of the subject.

In another aspect, provided herein is a method of sensitizing cancer cells to a tyrosine kinase inhibitor (TKI) in a subject in need thereof, comprising inhibiting a functional ATPase domain of RNF213 protein in the cells of the subject.

In another aspect, provided herein is a method of sensitizing cancer cells to a tyrosine kinase inhibitor (TKI) in a subject in need thereof, comprising inhibiting RNF213 protein expression or degrading RNF213 protein in the cells of the subject.

In another aspect, provided herein is a method of sensitizing cancer cells to a tyrosine kinase inhibitor (TKI) in a subject in need thereof, comprising inhibiting the RING domain of RNF213 protein in the cells of the subject.

In another aspect, provided herein is a method of sensitizing cancer cells to a tyrosine kinase inhibitor (TKI) in a subject in need thereof, comprising inhibiting oligomerization of RNF213 protein in the cells of the subject.

In another aspect, provided herein is a method of sensitizing cancer cells to a tyrosine kinase inhibitor (TKI) in a subject in need thereof, comprising inhibiting an RNF213 activator in the cells of the subject.

In another aspect, provided herein is a method of treating a cancer in a subject in need thereof or preventing recurrence of a cancer in a subject who has been treated with a targeted therapy, comprising activating the RZ domain of RNF213 protein in cancer cells of the subject.

In another aspect, provided herein is a method of treating a cancer in a subject in need thereof or preventing recurrence of a cancer in a subject who has been treated with a targeted therapy, comprising inhibiting a functional ATPase domain of RNF213 protein in cancer cells of the subject.

In another aspect, provided herein is a method of treating a cancer in a subject in need thereof or preventing recurrence of a cancer in a subject who has been treated with a a targeted therapy, comprising inhibiting the RING domain of RNF213 protein in cancer cells of the subject.

In another aspect, provided herein is a method of treating a cancer in a subject in need thereof or preventing recurrence of a cancer in a subject who has been treated with a targeted therapy, comprising inhibiting oligomerization of RNF213 protein in cancer cells of the subject.

In various embodiments, the tyrosine kinase inhibitor (TKI) is lapatinib, tucatinib, neratinib, afatinib, alectinib, axitinib, bosutinib, brigatinib, cabozantinib, ceritinib, crizotinib, dasatinib, entrectinib, erlotinib, gefitinib, imatinib, larotrectinib, lorlatinib, nilotinib, pazopanib, ponatinib, pyrotinib, regorafenib, sorafenib, sunitinib, or vandetanib, or a combination thereof. In some embodiments, the tyrosine kinase inhibitor (TKI) is lapatinib, tucatinib, or neratinib, or a combination thereof.

In various embodiments, the cells express human epidermal growth factor receptor 2 (HER2), HER4, epidermal growth factor receptor (EGFR), vascular endothelial growth factor receptor (VEGFR), platelet-derived growth factor receptor (PDGFR), rearranged during transfection (RET), c-ros oncogene 1 (ROS1), mesenchymal-epithelial transition factor (MET), tropomyosin receptor kinase (TRK), breakpoint cluster region-abelson murine leukemia viral oncogene homolog 1 (BCR-ABL), anaplastic lymphoma kinase (ALK), KIT proto-oncogene, receptor tyrosine kinase (KIT), sarcoma (Src) oncogene, or rapidly accelerated fibrosarcoma (RAF), or a combination thereof.

In various embodiments, the cancer is a breast cancer, a pancreatic cancer, a gastric cancer, a cervical cancer, a colorectal cancer, a head and neck cancer, a thyroid cancer, a prostate cancer, an ovarian cancer, non-small cell lung cancer (NSCLC), endometrial cancer, bladder cancer, melanoma, renal cell carcinoma, hepatocellular carcinoma, seminoma, gastrointestinal stromal tumor (GIST), glioblastoma, inflammatory myofibroblastic tumor (IMT), dermatofibrosarcoma protuberans, chronic myeloid leukemia (CML), or acute lymphoblastic leukemia (ALL). In some embodiments, the breast cancer is a human epidermal growth factor receptor 2 (HER2+) breast cancer.

In some embodiments of any of the above-described methods, the RNF213 activator is ABL1/2 or TNK1. In some embodiments, inhibiting ABL1/2 comprises administering to the subject an effective amount of asciminib, dasatinib, imatinib, AP 24149, ponatinib, ruserontinib, PD173952, bosutinib, rebastinib, olverembatinib, KW-2449, bafetinib, or nilotinib. In some embodiments, inhibiting TNK1 comprises administering to the subject an effective amount of TP-5801, TP-5801 TFA, XMD8-92, CZC-25146, or CZC-25146 hydrochloride.

In some embodiments of any of the above-described methods, degrading RNF213 protein comprises administering to the subject an effective amount of PROTAC or a molecular glue.

In some embodiments of any of the above-described methods, inhibiting RNF213 protein expression comprises administering to the subject an effective amount of an siRNA, an shRNA, an anti-sense oligonucleotide, or a site-specific nuclease.

In another aspect, provided herein is a method of treating a cancer in a subject in need thereof or preventing recurrence of a cancer in a subject who has been treated with a chemotherapy, radiotherapy, or a targeted therapy, comprising activating the RZ domain of RNF213 protein in cancer cells of the subject.

In another aspect, provided herein is a method of treating a cancer in a subject in need thereof or preventing recurrence of a cancer in a subject who has been treated with a chemotherapy, radiotherapy, or a targeted therapy, comprising activating a functional ATPase domain of RNF213 protein in cancer cells of the subject.

In another aspect, provided herein is a method of treating a cancer in a subject in need thereof or preventing recurrence of a cancer in a subject who has been treated with a chemotherapy, radiotherapy, or a targeted therapy, comprising inhibiting the RING domain of RNF213 protein in cancer cells of the subject.

In another aspect, provided herein is a method of treating a cancer in a subject in need thereof or preventing recurrence of a cancer in a subject who has been treated with a chemotherapy, radiotherapy, or a targeted therapy, comprising promoting oligomerization of RNF213 protein in cancer cells of the subject.

In another aspect, provided herein is a method for eliminating hypoxic tumor cells in a subject in need thereof, comprising activating the RZ domain of RNF213 protein in the tumor cells of the subject.

In another aspect, provided herein is a method for eliminating hypoxic tumor cells in a subject in need thereof, comprising activating a functional ATPase domain of RNF213 protein in the tumor cells of the subject.

In another aspect, provided herein is a method for eliminating hypoxic tumor cells in a subject in need thereof, comprising inhibiting the RING domain of RNF213 protein in the tumor cells of the subject.

In another aspect, provided herein is a method for eliminating hypoxic tumor cells in a subject in need thereof, comprising promoting oligomerization of RNF213 protein in the tumor cells of the subject.

In another aspect, provided herein is a method for enhancing effectiveness of an anti-cancer treatment in a subject in need thereof, comprising co-administering the anti-cancer treatment with an effective amount of an activator of the RZ domain of RNF213 protein. In some embodiments, co-administering comprises administering to the subject the anti-cancer treatment and an activator of the RZ domain of RNF213 protein simultaneously in one composition or in separate compositions. In some embodiments, co-administering comprises administering the anti-cancer treatment and an activator of the RZ domain of RNF213 protein sequentially, in any order.

In another aspect, provided herein is a method for enhancing effectiveness of an anti-cancer treatment in a subject in need thereof, comprising co-administering the anti-cancer treatment with an effective amount of an activator of a functional ATPase domain of RNF213 protein. In some embodiments, co-administering comprises administering the anti-cancer treatment and the activator of the functional ATPase domain of RNF213 protein simultaneously in one composition or in separate compositions. In some embodiments, co-administering comprises administering the anti-cancer treatment and the activator of the functional ATPase domain of RNF213 protein sequentially, in any order.

In another aspect, provided herein is a method for enhancing effectiveness of an anti-cancer treatment in a subject in need thereof, comprising co-administering the anti-cancer treatment with an effective amount of an inhibitor of the RING domain of RNF213 protein. In some embodiments, co-administering comprises administering the anti-cancer treatment and an inhibitor of the RING domain of RNF213 protein simultaneously in one composition or in separate compositions. In some embodiments, co-administering comprises administering the anti-cancer treatment and an inhibitor of the RING domain of RNF213 protein sequentially, in any order.

In another aspect, provided herein is a method for enhancing effectiveness of an anti-cancer treatment in a subject in need thereof, comprising co-administering the anti-cancer treatment with an effective amount of a compound promoting oligomerization of RNF213 protein in cancer cells of the subject. In some embodiments, co-administering comprises administering the anti-cancer treatment and the compound promoting oligomerization of RNF213 protein simultaneously in one composition or in separate compositions. In some embodiments, co-administering comprises administering the anti-cancer treatment and the compound promoting oligomerization of RNF213 protein sequentially, in any order.

In some embodiments of any of the above-described methods, the anti-cancer treatment is a chemotherapy, targeted therapy, or antibody-drug conjugate (ADC) treatment.

In some embodiments of any of the above-described methods, the cancer is a breast cancer, pancreatic cancer, or cervical cancer. In some embodiments, the breast cancer is a HER2+ breast cancer.

In some embodiments of any of the above-described methods, the cancer has significant regions of hypoxia.

In some embodiments of any of the above-described methods, the functional ATPase domain of RNF213 protein is the 3rd or 4th ATPase domain.

In some embodiments of any of the above-described methods, an activator of a functional ATPase domain is a PTP1B inhibitor.

In some embodiments of any of the above-described methods, the subject is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H show RNF213 phosphorylation at Y1275 is regulated by PTP1B and ABL1/2 and controls oligomerization. In FIG. 1A RNF213 Y1275 phosphorylation regulates interaction with PTP1B substrate-trapping mutant. BT474 RNF213-KO-PTPN1-KD cells, with or without stable expression of a doxycycline (Dox)-inducible, HA-tagged substrate trapping mutant (D181A) of mouse PTP1B (HA-Ptpn1^{D/A}), were transiently transfected with 3×-FLAG-RNF213 or 3×-FLAG-RNF213^{Y1275F} and exposed to 0.5 μg/ml Dox (+) or left untreated (−). Lysates (left panel) were subjected to anti-HA immunoprecipitation and immunoblotting for the indicated proteins (right panel). ERK2 serves as a loading control. In FIG. 1B, Y1275 is phosphorylated in BT474 cells. BT474 RNF213-KO-PTPN1-KD cells were transiently transfected with 3×-FLAG-RNF213 or 3×-FLAG-RNF213^{Y1275F}. Lysates were subjected to anti-FLAG immunoprecipitation and immunoblotting, as indicated, and pY/FLAG signal (each in arbitrary units) was quantified. In FIG. 1C, Y1275 phosphorylation promotes RNF213 oligomerization. BT474 RNF213-KO-PTPN1-KD cells were transiently transfected with GFP-RNF213 and 3×-FLAG-RNF213 or 3×-FLAG-RNF213^{Y1275F}. Lysates were subjected to anti-FLAG immunoprecipitation/immunoblotting. Vinculin serves as a loading control. In FIG. 1D, ABL kinases promote RNF213-mediated hypoxia hypersensitivity. BT474 and BT474 PTPN1-KO cells were treated with the indicated kinase inhibitors. Cells were subjected to 0.1% O₂ for 55 hours, and viable cells were counted. Error bars represent ±S.E.M. In FIG. 1E, Dasatinib inhibits RNF213-PTPTB interaction. BT474 RNF213-KO-PTPN1-KD cells expressing substrate trapping mutant HA-Ptpn1^{D/A} were transiently transfected with 3×-FLAG-RNF213 and then treated with dasatinib or vehicle (DMSO) for 24 hours. Cell lysates were subjected to anti-HA immunoprecipitation/immunoblotting. In FIG. 1F, Dasatinib inhibits tyrosine phosphorylation of RNF213. BT474 RNF213-KO-PTPN1-KD cells were transiently transfected with 3×-FLAG-RNF213 followed by treatment with DMSO or dasatinib for 24 hours, and lysates were subjected to anti-FLAG immunoprecipitation/immunoblotting. In FIG. 1G, ABL phosphorylates RNF213 on Y1275. 3×-FLAG-RNF213 and 3×-FLAG-RNF213^{Y1275F} were purified and phosphorylated in vitro with the indicated amounts of recombinant ABL1. In FIG. 1H, ABL1/ABL2 promotes RNF213 mediated hypoxia hypersensitivity in PTPN1-KO cells. BT474 and BT474 PTPN1-KO cells were transfected with ABL1 and/or ABL2 siRNAs targeting or control non-targeting siRNA. Cells were subjected to 0.1% O₂ for 55 hours, and viable cells were counted (top panel). Error bars represent ±S.E.M. Bottom panel shows immunoblots for the indicated proteins. All experiments (FIGS. 1A-1G) are representative results from three independent experiments except for the quantifications in FIGS. 1B, 1D, 1F, and 1H, which are pooled data from the triplicates. Panels FIGS. 1B and 1F were analyzed by ratio paired two-tailed t-test; all other results were evaluated by two-way ANOVA (analysis of variance), *P<0.05, ****P<0.0001, ns=non-specific band.

FIGS. 2A-2F illustrate RNF213 interacts with CYLD/SPATA2. FIG. 2A is a schematic showing proximity-based labelling of RNF213 interactors. HeLa FlpIn-TRex cells expressing TurboID (control), TurboID-RNF213 (N-Turbo) or RNF213-TurboID (C-Turbo) were used for proximity-based biotin labeling. FIG. 2B is a one-sided volcano plot showing the fold-change and SAINT score of proteins identified in C-Turbo versus Control Turbo. Proteins with a SAINT Score >5% FDR are highlighted and considered to be potential interacting partners of RNF213. FIG. 2C shows that CYLD and SPATA2 are in proximity with RNF213. Lysates prepared as in FIG. 2A were subjected to streptavidin affinity purification and immunoblotting for the indicated proteins. FIG. 2D shows that CYLD and SPATA2 interact with RNF213. BT474 RNF213-KO-PTPN1-KD cells were transiently transfected with 3×-FLAG-RNF213, and lysates were subjected to immunoprecipitation and immunoblotting as indicated. FIGS. 2E and 2F show CYLD and SPATA2 protect against hypoxia hypersensitivity. BT474 and BT474 PTPN1-KO cells, with or without CYLD, were subjected to 0.1% O₂ for indicated times, and viable cells were counted (top panel). Immunoblots (bottom panels) show absence of CYLD/SPATA2 in stable pools generated with the indicated sgRNAs. Error bars represent ±S.E.M. FIGS. 2B, 2E, and 2F are pooled results from three biological replicates; all other data are representative of these replicates, ****P<0.0001, two-way ANOVA.

FIGS. 3A-3E demonstrate the role of RNF213 RZ and RING domains in CYLD and SPATA2 ubiquitylation. FIG. 3A illustrates that RNF213 promotes SPATA2 and CYLD ubiquitylation. BT474 and BT474 RNF213-KO cells were co-transfected with expression constructs for HA-ubiquitin (HA-UB) and ALFA-SPATA2 or ALFA-CYLD and treated with claramine or vehicle (−), as indicated. Lysates (left panel) were subjected to anti-ALFA immunoprecipitation/immunoblotting (right panel). FIGS. 3B-3E demonstrate opposing actions of RNF213 RZ and RING domains in CYLD and SPATA2 ubiquitylation. Purified wild type or mutant 3×-FLAG-RNF213 was added to in vitro ubiquitylation assays with UBE2L3, CYLD, and/or SPATA2 and immunoblotted, as indicated. Each blot is representative of two biological replicates, ns=non-specific band.

FIGS. 4A-4E demonstrate RNF213 RING mutants/highly penetrant MMD SNPs activate RZ domain and require ATPase activity. In FIGS. 4A-4D, BT474 RNF213-KO cell reconstituted with RNF213^{WT}, the indicated RNF213 mutants, parental BT474 cells, and BT474 RNF213-KO cells with or without PTP1B, were subjected to 0.1% O₂ for 40 hours. Immunoblotting was performed for the indicated proteins. In FIG. 4E, RZ and ATPase domains of RNF213 are required for hypoxia hypersensitivity of BT474 PTPN1-KO cells. Cells prepared as in FIGS. 4A-4D were subjected to 0.1% O₂, and viable cells were counted after 55 hours. Error bars represent ±S.E.M. (n=3 biological replicates, each panel) and each blot is representative of two biological replicates. ****P<0.0001, two-way ANOVA, ns=non-specific band.

FIGS. 5A-5H illustrate PTPN1-KO cells dying via RNF213- and LUBAC-dependent pyroptosis. FIG. 5A shows that wild-type, but not RING-dead RNF213 requires LUBAC for CYLD/SPATA2 degradation. BT474 RNF213-KO cells reconstituted with wild-type RNF213 or RNF213^{H4014A} parental BT474 cells and BT474 RNF213-KO with or without PTP1B were transfected with RBCK1 or control siRNA. Cells were subjected to 0.1% 02 for 40 hours, lysed, and, immunoblotted as indicated (top panel). RBCK1 knockdown was confirmed by RT-qPCR (bottom panel). In FIG. 5B, RING-dead RNF213 bypasses LUBAC requirement. The indicated cells were subjected to 0.1% O₂ for 55 hours, and viable cell numbers were counted. FIG. 5C shows that RNF213 promotes NF-κB activity. Cells described in FIG. 4E were transfected with an NF-κB reporter construct and subjected to 0.1% O₂ for 48 hours. Firefly and Renilla luciferase signals were measured, reporter activity was quantified as the ratio of Firefly/Renilla signal, and all other signals were normalized to that from parental BT474 cells. FIG. 5D shows hypoxic cell death in PTPN1-KO cells is caspase-dependent. BT474 and BT474 PTPN1-KO cells were treated with the indicated inhibitors, placed in 0.1% O₂ for 55 hours, and cell viability was assessed. FIGS. 5E and 5F provide hypoxic cell death in PTPN1-KO cells requires inflammatory caspases and the inflammasome, as in FIG. 5D, except with/without VX765 (FIG. 5E) or MCC950 (FIG. 5F). FIG. 5G shows GSDMD promotes hypoxic cell death of PTPN1-KO cells. Pools of BT474 and BT474 PTPN1-KO cells, with or without GSDMD deletion, were subjected to 0.1% O₂, and cell viability was measured at 55 hours (top panel). Immunoblot indicates efficiency of GSDMD knockout (bottom panel). FIG. 5H shows hypoxia-hypersensitive cells release IL-10. Cells prepared as in FIG. 4E were subjected to 0.1% O₂ for 55 hours, and IL-1β in supernatants was quantified by immunoassay. In all panels, error bars represent ±S.E.M. (n=3 biological replicates) and each blot is representative of two biological replicates. ****P<0.0001, two-way ANOVA, ns=non-specific band.

FIGS. 6A-6I show hypoxia-evoked ER stress activates the inflammasome for RNF213-triggered pyroptosis, in accordance with an exemplary embodiment of the present disclosure. FIGS. 6A and 6B show that ER stress promotes hypoxia hypersensitivity. BT474 and BT474 PTPN1-KO cells treated with the IRE1α inhibitor 4μ8C (FIG. 6A) or the PERK inhibitor GSK2606414 (FIG. 6B), were subjected to 0.1% 02 for 55 hours, and viability was assessed. FIG. 6C demonstrates that ER stress causes pyroptosis in CYLD/SPATA2-deficient cells in normoxia. BT474 cells, with or without CYLD/SPATA2, were treated with tunicamycin (10 μM) or thapsigargin (5 μM) for 40 hours, and cell viability was measured at 40 hours. Parental and PTPN1-KO BT474 cells, with or without CYLD- or NLRP3-KO, were injected subcutaneously into athymic nu nu mice (6/group) (FIGS. 6D and 6E). Tumor size was measured weekly for 9 weeks (FIG. 6D). Residual tumors were excised, fixed, and stained with H&E (FIG. 6E). RNF213-KO-sgPTPN1 BT474 cells were stably reconstituted with inducible vectors expressing WT RNF213 or the indicated RNF213 mutants and implanted into athymic nu nu mice (6/group) (FIGS. 6F and 6G). When tumors reached 100 mm³, mice were switched to doxycycline (Dox) chow, and tumor size was measured weekly (FIG. 6F). Residual tumors were excised, fixed, and stained with H&E (FIG. 6G). * in FIGS. 6E and 6G indicates necrosis, with necrotic area quantified at right. Lysates from FIGS. 6D and 6E were subjected to immunoblotting for pyroptosis markers (FIGS. 6H and 6I). Error bars represent ±S.E.M (n=3 biological replicates), ****P<0.0001, two-way ANOVA.

FIGS. 7A-7E demonstrate Y1275 phosphorylation is regulated by PTP1B and controls RNF213 oligomerization, in accordance with an exemplary embodiment of the present disclosure. FIG. 7A shows PTP1B inhibitor sensitizes multiple breast cancer subtypes to hypoxia. The indicated cell lines were treated with the PTP1B inhibitor claramine, placed in 0.1% O₂ for 55 hours, and viable cells were counted. FIG. 7B shows spectrum supporting phosphorylation of RNF213-Y1275 on peptide EAAEPLSEPKEDQEAAELLSEPEEESERHILELEEVYDYLYQPSYR (SEQ ID NO: 4590), from 3×-FLAG-RNF213-TurboID-enriched samples. The HCD MS/MS spectra of sextuply charged m/z 922.9244 ion recorded in the ion trap of an Orbitrap Eclipse is shown. Observed N-terminal sequence ions (b-ions) are shown in blue, and C-terminal fragment ions (y-ions) are shown in red. Backbone cleavages are indicated in the peptide sequence, and the phosphorylated tyrosine is indicated (red). Figure discloses SEQ ID NOS 18 and 4661, respectively, in order of appearance. FIG. 7C provides Y1275 phosphorylation affects RNF213-PTP1B interaction. AU565 RNF213-KO-PTPN1-KD cells with or without stable expression of doxycycline (Dox)-inducible, HA-Ptpn1^{D/A} were transiently transfected with 3×-FLAG-RNF213 or 3×-FLAG-RNF213^{Y1275F} and exposed to 0.5 μg/ml Dox (+) or left untreated (−). Lysates (top panel) were subjected to anti-HA immunoprecipitation and immunoblotting for the indicated proteins (bottom panel). ERK2 serves as a loading control. FIG. 7D shows RNF213-Y1275 is also phosphorylated in AU565 cells. AU565 RNF213-KO-PTPN1-KD cells were transiently transfected with 3×-FLAG-RNF213 or 3×-FLAG-RNF213^{Y1275F}. Lysates were subjected to anti-FLAG immunoprecipitation and immunoblotting, as indicated, and pY/FLAG signal (each in arbitrary units) was quantified. FIG. 7E shows Y1275 phosphorylation promotes RNF213 oligomerization. AU565 RNF213-KO-PTPN1-KD cells were transiently transfected with expression vectors for GFP-RNF213 and 3×-FLAG-RNF213 or 3×-FLAG-RNF213^{Y1275F}. Lysates were subjected to anti-FLAG immunoprecipitation/immunoblotting. Vinculin serves as a loading control. Error bars represent ±S.E.M. FIGS. 7A and 7D (lower panels) are pooled results from three biological replicates; all other data except (FIG. 7B) are representative of these replicates. FIGS. 7A and 7E were evaluated by two-way ANOVA, whereas FIG. 7D was analyzed by ratio paired two-tailed t-test, **P<0.01, ***P<0.001, ****P<0.0001, ns=non-specific band.

FIGS. 8A-8G show ABL phosphorylates RNF213 on Y1275, in accordance with an exemplary embodiment of the present disclosure. FIG. 8A demonstrate that SKBR3 and SKBR3 PTPN1-KO cells were treated with the indicated inhibitors. Cells were subjected to 0.1% O₂ for 36 hours, and viable cells were counted. FIG. 8B illustrate that Dasatinib inhibits RNF213-PTP1B interaction. AU565 RNF213-KO-PTPN1-KD cells expressing substrate trapping mutant HA-Ptpn1^{D/A} were transiently transfected with expression vector for 3×-FLAG-RNF213, then treated with dasatinib or vehicle (DMSO) for 24 hours. Lysates were subjected to anti-HA immunoprecipitation/immunoblotting. FIG. 8C shows Dasatinib inhibits tyrosine phosphorylation of RNF213. AU565 RNF213-KO-PTPN1-KD cells were transiently transfected with 3×-FLAG-RNF213 followed by treatment with DMSO or dasatinib for 24 hours. Lysates were subjected to anti-FLAG immunoprecipitation/immunoblotting. FIG. 8D provides purity of 3×-FLAG-RNF213 and 3×-FLAG-RNF21^{Y1275F} proteins. FIG. 8E shows that ABL phosphorylates RNF213 on Y1275. Purified 3×-FLAG-RNF213 and 3×-FLAG-RNF213^{Y1275F} were phosphorylated in vitro with recombinant His-ABL1 for the indicated times. FIG. 8F shows that ABL1/ABL2 promote RNF213-mediated hypoxia hypersensitivity in PTPN1-KO cells. MDA-MB-361 and MDA-MB-361 PTPN1-KO cells were transfected with siRNAs targeting ABL1 and/or ABL2 or control non-targeting siRNA. Cells were subjected to 0.1% O₂ for 48 hours, and viable cells were counted (top panel). Immunoblot shows knockdown efficiency (bottom panel). Error bars represent ±S.E.M. All panels (FIGS. 8A-8F) are representative results from two independent experiments, except for the quantifications in FIGS. 8A, 8C, and 8F, which are pooled data from triplicates. Purified 3×-FLAG-RNF213 was phosphorylated in vitro with recombinant His-ABL1 for 10 minutes, followed by anti-FLAG immunoprecipitation and incubation with recombinant GST-PTP1B in the presence or absence of the PTP inhibitor sodium orthovandate (FIG. 8G). FIGS. 8A and 8F were evaluated by two-way ANOVA; FIG. 8C was analyzed by ratio paired two-tailed t-test **P<0.01, ****P<0.0001, ns=non-specific band.

FIGS. 9A-9E provide sample preparation for proximity-based labelling screen, in accordance with an exemplary embodiment of the present disclosure. FIG. 9A shows HeLa FlpIn-TRex cells reconstituted with Control Turbo, N-turbo, or C-turbo were induced with 0.5 μg/ml of Dox. Post-biotin labelling, lysates (left panel) were subjected to streptavidin affinity purification (right panel) and immunoblotting. In FIG. 9B, silver-stained SDS-PAGE gel showing that comparable amounts of streptavidin-purified proteins were submitted for mass spectrometry. FIG. 9C is a one-sided volcano plot showing fold-change and SAINT score of proteins identified in N-Turbo versus Control Turbo. Proteins with SAINT Score >5% FDR are highlighted. FIGS. 9C and 9E provide GO pathway analysis showing top 10 enriched molecular functions of C-Turbo (FIG. 9D)- and N-Turbo (FIG. 9E)-interacting proteins.

FIGS. 10A-10H demonstrate that CYLD/SPATA2 are relevant substrates of RNF213 in mediating hypoxia hypersensitivity, in accordance with an exemplary embodiment of the present disclosure. FIGS. 10A and 10B show SKBR3 and SKBR3 PTPN1-KO cells, with or without CYLD (FIG. 10A) or SPATA2 (FIG. 10B), subjected to 0.10% O₂ for the indicated times, and viable cells were counted (top panels). Immunoblots (bottom panels) show absence of CYLD/SPATA2 in stable pools generated with the indicated sgRNAs. FIG. 10C shows that RNF213 promotes SPATA2 and CYLD ubiquitylation. AU565 and AU565 RNF213-KO cells were co-transfected with expression constructs for HA-UB and ALFA-SPATA2 or ALFA-CYLD and treated with claramine or vehicle (−), as indicated. Lysates (left panel) were subjected to anti-ALFA immunoprecipitation/immunoblotting (right panel). FIG. 10D provides purity of 3×-FLAG-RNF213, 3×-FLAG-RNF21^{H4509A}, 3×-FLAG-RNF21^{H4014A}, 3×-FLAG-RNF21^{H4014A/H4509A} after indicated steps. FIGS. 10E and 10F show effects of RNF213 mutants on global ubiquitylation. Hela FlpIn-TRex RNF213-KO (FIG. 10E) and BT474 RNF213-KO (FIG. 10F) cells were co-transfected with expression constructs for HA-UB and 3×-FLAG-tagged RNF213 or -RNF213^{H4014A}-RNF213^{H4509A} or -RNF213^{H4014A/H4509A}. Cells were subjected to 0.1% O₂ for the indicated times, and lysates were subjected to immunoblotting. FIGS. 10G and 10H show in vitro ubiquitylation assays with CYLD (FIG. 10G) or SPATA2 (FIG. 10H) using wild type or K63R ubiquitin. CYLD (FIG. 10G) and SPATA2 (FIG. 10H) were recovered and immunoblotted, as indicated. Error bars represent S.E.M. FIGS. 10A and 10B are pooled results from three biological replicates; data in FIGS. 10C, 10E, and 10F are representative of replicates, ****P<0.0001, two-way ANOVA, ns=non-specific bands.

FIGS. 11A-11F demonstrate RNF213 RING mutants/highly penetrant MMD SNPs activate RZ domain and require ATPase activity, in accordance with an exemplary embodiment of the present disclosure. FIGS. 11A through 11D show AU565 RNF213-KO cell reconstituted with RNF213^{WT} or the indicated RNF213 mutants, parental AU565 cells, and AU565 RNF213-KO cells with or without PTPN1 deletion, subjected to 0.1% O₂ for 40 hours. Immunoblotting was performed for the indicated proteins. FIG. 11E show RZ and ATPase domains of RNF213 are required for hypoxia hypersensitivity of AU565 PTPN1-KO cells. Cells prepared as in FIGS. 11A-11D were subjected to 0.1% O₂, and viable cells were counted after 48 hours. RNF213-KO BT474 cells reconstituted with RNF213 or RNF213^{Y1275F} were treated with 20 μM cycloheximide (CHX) for the indicated times. MG132 (10 μM), 100 μM CQ (chloroquine), or vehicle (DMSO) was added for the last 3 hours, as indicated, and lysates were immunoblotted for the indicated proteins (FIG. 11F). Error bars represent ±S.E.M (n=3 biological replicates, each panel); each blot is representative of two biological replicates. ***P<0.001, ****P<0.0001, two-way ANOVA, ns=non-specific band.

FIGS. 12A-12K demonstrate PTPN1-KO cell death via RNF213- and LUBAC-dependent pyroptosis, in accordance with an exemplary embodiment of the present disclosure. FIG. 12A shows that wild-type, but not RING-dead, RNF213 requires LUBAC for CYLD/SPATA2 degradation. AU565 RNF213-KO cells reconstituted with wild-type RNF213 or RNF213^{H4014A}, parental AU565 cells, and AU565 RNF213-KO cells with or without PTPN1 deletion were transfected with RBCK1 or control siRNAs. Cells were subjected to 0.1% 02 for 40 hours, lysed, and immunoblotted as indicated (top panel). RBCK1 knockdown was confirmed by RT-qPCR (bottom panel). FIG. 12B demonstrates RING-dead RNF213 bypasses LUBAC requirement. The indicated cells were subjected to 0.1% O₂ for 48 hours, and viable cell numbers were counted. FIG. 12C shows RNF213 promotes NF-κB activity. The cells described in FIG. 11E were transfected with an NF-κB reporter construct and subjected to 0.1% O₂ for 40 hours. Firefly and Renilla luciferase signals were measured, reporter activity was quantified as the ratio of Firefly/Renilla signal, and all signals were normalized that from parental AU565 cells. FIGS. 12D and 12E demonstrate hypoxic cell death in PTPN1-KO cells is caspase-dependent. SKBR3 and SKBR3 PTPN1-KO (FIG. 12D) and MDA-MB-361 and MDA-MB-361 PTPN1-KO (FIG. 12E) cells were treated with the indicated inhibitors, placed in 0.1% O₂ for 40 (FIG. 12D) or 48 (FIG. 12E) hours, and cell viability was assessed. FIGS. 12F and 12G demonstrate hypoxic cell death in MDA-MB-361 PTPN1-KO cells requires inflammatory caspases and the inflammasome. As in (FIG. 12E), except with/without VX765 (FIG. 12F) or MCC950 (FIG. 12G). FIG. 12H shows GSDMD promotes hypoxic cell death of PTPN1-KO cells. Pools of MDA-MB-361 and MDA-MB-361 PTPN1-KO cells, with or without GSDMD deletion, were subjected to 0.1% O₂, and cell viability was measured at 48 hours (top panel). Immunoblot indicates efficiency of GSDMD knockout (bottom panel). IL-1β (FIG. 12J) and % maximal LDH release (FIG. 12K) were quantified in supernatants from the indicated hypoxic cells. FIG. 121 demonstrates hypoxia-hypersensitive cells release IL-1β. Cells prepared as in FIG. 12E were subjected to 0.1% 02 for 48 hours, and IL-10 in supernatants was analyzed by immunoassay. In all panels, error bars represent ±S.E.M. (n=3 biological replicates), and each blot is representative of two biological replicates. ***P<0.001, ****P<0.0001, two-way ANOVA, ns=non-specific band.

FIGS. 13A-13L demonstrate hypoxia-evoked ER stress activates the inflammasome for RNF213-triggered pyroptosis, in accordance with an exemplary embodiment of the present disclosure. FIGS. 13A and 13B show ER stress promotes hypoxia hypersensitivity. MDA-MB-361 and MDA-MB-361 PTPN1-KO cells treated with the IRE1α inhibitor 4μ8C (FIG. 13A) or the PERK inhibitor GSK2606414 (FIG. 13B), were subjected to 0.1% O₂ for 48 hours, and viability was assessed. FIG. 13C shows ER stress causes pyroptosis in CYLD/SPATA2-deficient cells in normoxia. MDA-MB-361 cells, with or without CYLD/SPATA2, were treated with tunicamycin (10 μM) or thapsigargin (5 μM) for 40 hours and viability was assessed. For FIGS. 13D and 13E, ERN1, HSPA5, and DDIT3 mRNA levels in parental and PTPN1-KO BT474 cells subjected to 0.1% O₂ were quantified by qRT-PCR. DDIT3 levels were also assessed in parental and PTPN1-KO BT474 cells transfected with DDIT3 or control siRNAs for 24 hours (FIG. 13E). Cells transfected with siRNAs as in FIG. 13E were subjected to 0.1% O₂ for 48 hours, and viable cells were counted (FIG. 13F). Parental and PTPN1-KO BT474 cells were transfected with an NF-κB reporter construct and subjected to 0.10% O₂ for 48 hours (FIG. 13G). Reporter activity was quantified as the ratio of Firefly/Renilla signal, normalized to that in parental cells. Expression of the indicated genes was assessed by qRT-qPCR (FIG. 13H). Same as in FIG. 13G and FIG. 13H, except that the cognate MDA-MB-361 cells were analyzed (FIGS. 131 and 13J). For FIGS. 13K and 13L, BT474 cells with or without CYLD/SPATA2 were treated with nigericin (20 μM) for 6 hours, and cell viability (FIG. 13K) and LDH release (FIG. 13L) were assessed. Error bars represent ±S.E.M. (n=3 biological replicates), ****P<0.0001, two-way ANOVA.

FIG. 14 is a schematic model for PTP1B/RNF213-mediated hypoxia hypersensitivity. Left panel: ABL1/2-catalyzed phosphorylation of RNF213 at Y1275 promotes RNF213 oligomerization; PTP1B dephosphorylates this site. Oligomerized RNF213, via its RZ domain, ubiquitylates CYLD and SPATA2, and further ubiquitylation, catalyzed by the LUBAC complex, promotes CYLD/SPATA2 degradation. Left middle panel: ATPase-dead RNF213 cannot oligomerize and does not promote hypoxia hypersensitivity. Right middle panel: RING-dead RNF213 enhances RZ-mediated ubiquitylation of CYLD/SPATA2 and leads to CYLD and SPATA2 degradation even in the absence of LUBAC. Degradation of CYLD/SPATA2 leads to increased NF-κB signaling, priming pyroptosis. Hypoxia-associated ER-stress provides the “second signal” to activate the inflammasome formation, leading to RNF213-driven pyroptotic cell death. Right panel: RZ-dead or RING/RZ-dead RNF213 cannot ubiquitylate CYLD/SPATA2 and does not promote hypoxia hypersensitivity in response to PTP1B inhibition.

FIGS. 15A and 15B provide schematic domain map and domain amino acid sequences of RNF213. For the relevance of RNF213 domains or amino acid residues (see also, e.g., Table 6): (1) Y1275 phosphorylation by ABL1/2 promotes RNF213 oligomerization. This site is dephosphorylated by PTP1B; (2) ATPase domains AAA3 and AAA4 are functional ATPase domains required for RNF213 oligomerization; (3) K2426, K2775 or E2845 are critical amino acid sites required for ATPase activity and RNF213 oligomerization; (4) H4014 is catalytic site for RING domain activity of RNF213 which may be required for ubiquitylation; (5) H4509 is a catalytic site for RZ domain activity of RNF213 which can be required for ubiquitylation; (6) C9997Y is a highly penetrant MMD SNP; (7) R4810K is the most prevalent but low penetrance MMD SNP. The present disclosure demonstrates RING-impaired (H4014A) RNF213 has a hyperactive RZ domain, but this can additionally require RNF213 oligomerization (e.g., in context with CYLD and SPATA2 as substrates). Since C3997Y SNP is also in the RING domain of RNF213 and it functions similarly to RING dead RNF213 (H4014A), the data described herein predicts that MMD SNPs will also be a gain of function of RZ domain.

FIG. 16 is a plot showing the number of drug-tolerant persister cells (DTPs) after HER2 inhibitor treatment comparing wild type to RNF213 knock out.

FIG. 17 demonstrates that TNK1 promotes global ubiquitylation upon PTP1B inhibition. BT474 cells were treated with DMSO or PTP1B inhibitor (Claramine, 10 PM). After 24 hours of seeding the cells, siControl or siTNK1 were transfected into the cells with either DMSO or PTP1B inhibitor. Lysates prepared were subjected to immunoblotting for the indicated proteins.

FIG. 18 demonstrates that TNK1 promotes RNF213-mediated hypoxia hypersensitivity in PTPN1-KO cells. BT474 and BT474 PTPN1-KO cells (top) or MDA-MB-361 and MDA-MB-361 PTPN1-KO cells (bottom) were transfected with TNK1 siRNAs targeting or control non-targeting siRNA. Cells were subjected to 0.1% O₂ for 55 hours for BT474 (top) or 48 hours for MDA-MB-361 (bottom), and viable cells were counted.

FIG. 19 demonstrates that TNK1 phosphorylates RNF213. 3×-FLAG-RNF213 was purified and phosphorylated in vitro with the indicated amounts of recombinant TNK1.

FIG. 20 illustrates PTP1B inhibition sensitized cancer cells to hypoxia. The indicated cell lines were treated with the PTP1B inhibitor claramine, placed in 0.1% O₂, and viable cells were counted. Pooled results from three biological replicates are shown; each data point represents the mean of triplicates from each experiment. Error bars represent +S.E.M.**P<0.01, ***P<0.001, ****P<0.0001, two-way ANOVA, ns=not significant.

FIGS. 21A-21E demonstrate that the PTP1B/RNF213/CYLD/SPATA2 pathway controls HER2+ breast tumorigenesis. For FIGS. 21A and 21B, parental and PTPN1-KO MDA-MB-361 cells, with or without CYLD-KO, were injected subcutaneously into athymic nu nu mice (6/group). Tumor size was measured weekly for 6 weeks (FIG. 21A). Residual tumors were excised, fixed, and stained with H&E (FIG. 21B). *representative necrotic regions. Tumors as described in FIGS. 6D and 6E were excised, fixed, and immunostained with Ki67 (top), HIF1α (middle), and EF5 (bottom) (FIG. 21C). ERN1, HSPA5, and DDIT3 mRNAs from the core or surface of parental and PTPN1-KO BT474 xenografts (3 each) were measured by qRT-PCR (FIG. 21D). Tumors from FIG. 6F were immunoblotted for the indicated proteins (FIG. 21E). **P<0.01, ***P<0.001, ****P<0.0001, two-way ANOVA with Dunnett's (FIG. 21A) or Tukey's (FIG. 21D) correction. Immunoblot, H & E images, and EF5 immunofluorescence are from one, and immunohistochemical staining (Ki67, HIF1α) are from two, sets of tumors. For gel source data, see FIG. 20.

FIG. 22 demonstrates that RNF213-KO sensitizes HER2+ breast cancer (BC) cells to HER2 tyrosine kinase inhibitor (TKI). Parental and RN % AF2I3-KO HER2+ breast cancer cell lines were treated with 1.2 gmol/L tucatinib (blue) or with 2.5 μmol/L lapatinib (red), with fresh media+inhibitors replaced every 3 days. Viable cells were counted after 15 days of treatment. Mean±SEM from three independent experiments is shown.

FIG. 23 demonstrates that the RNF213 RING-domain and ATPase-domain are required for drug-tolerant persister cancer cell (DTP) formation, Parental BT474 cells and RNF213-K0 BT474 cells stably reconstituted with RNF213 WT or the indicated RNF213 mutants were treated with 1.2 μmol/L tucatinib (blue) or with 2.5 μmol/L lapatinib (red), with fresh media+inhibitors replaced every 3 days. Viable cells were counted after 15 days of treatment. Mean±SEM from three independent experiments is shown.

### DETAILED DESCRIPTION

### Definitions

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The terms “inhibit” or “inhibition” as used herein refer to reducing a function or activity to an extent sufficient to achieve a desired biological or physiological effect. Inhibition may be complete or partial.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

“Drug-tolerant persister cells” or “drug-tolerant persister cancer cells” or “DTPs”, or the like, as used herein refer to a sub-population of cancer cells that survive under anti-cancer treatment(s), e.g., chemotherapy, radiation therapy, and/or targeted therapy.

It must also be noted that, as used in the specification and the appended claims, the singular forms “a,” “an,” and “the” include plural references, unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents that operate in a similar manner to accomplish a similar purpose.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

Most tumors have hypoxic or anoxic areas¹, and cancer cells adapt to limited oxygen in the tumor microenvironment (TME) by successively activating several signaling pathways. In normoxia, the transcription factors HIF1α and HIF2α (hereafter “HIF”) are hydroxylated by oxygen-dependent prolyl hydroxylases (PHD 1-3), bound by the Van Hippel Lindau tumor suppressor gene product (VHL), and targeted for ubiquitylation and proteasomal degradation². PHD enzymes are inhibited in low oxygen, resulting in HIF stabilization and induction of hypoxia response genes. More severe hypoxia (≤1% O₂) triggers the AMP-dependent kinase (AMPK) and endoplasmic reticulum (ER) stress pathways, which evoke distinct adaptive responses^{3,4}. The activity of NF-κB, a key transcription factor controlling inflammation and innate immunity, also increases in hypoxia⁵ via (an) incompletely defined mechanism(s) that include(s) decreased PHD regulation of NF-κB pathway signaling components. Conversely, NF-κB is required for basal HIF expression, and thus directly and indirectly regulates the transcriptome of hypoxic cells⁶. The complex interplay between HIF-induced, metabolic (AMPK), integrated stress response (ER stress), and inflammatory/innate immune^{7,8}(NF-κB) pathways helps determine cancer cell sensitivity to conventional (chemoradiation) and targeted therapy³, as well as whether tumor cell death evoked by antineoplastic therapies is “sterile” or immunogenic⁹⁻¹¹.

The protein-tyrosine phosphatase PTP1B (encoded by PTPN1) resides on the cytosolic surface of the endoplasmic reticulum (ER), where it dephosphorylates incoming receptor tyrosine kinases (RTKs) and cytokine receptors^{12,13}. Best known as a negative regulator of insulin and leptin signaling¹⁴⁻¹⁷, PTP1B is also required for Neu (encoding rodent HER2)-induced breast cancer in mice^{18,19}. Moreover, PTPN1 is amplified (˜5%) and overexpressed (˜72%) in human cancer^{20,21}. It was previously reported that PTP1B deficiency or inhibition increased the death of HER2+ breast cancer cells maintained at ≤1% O₂ without altering the HIF, ER stress, or AMPK pathways²². Instead, hypoxia hypersensitivity required RNF213, a large (˜600 kDa) protein with AAA-ATPase and E3 ligase domains. PTP1B deficiency/inhibition promoted an increase in RNF213-dependent global ubiquitylation, and RNF213 was tyrosine phosphorylated and appeared to be a PTP1B substrate. However, key details, including exactly how PTP1B regulates RNF213 and the mechanism and type of RNF213-dependent cell death, remained unclear.

Single nucleotide polymorphisms (SNPs) in RNF213 are associated with Moyamoya disease (MMD), a rare syndrome characterized by precocious carotid artery stenosis and stroke, often in teenagers or young adults²³⁻²⁵. RNF213 features six AAA-ATPase modules, two of which are active, a RING domain, a recently discovered “RZ” E3 ligase domain, and large unstructured regions²⁶⁻³⁰. In an AAA-ATPase- and ISG15-dependent manner, RNF213 can oligomerize into a hexameric, ˜3.6 mDa complex^{26,28}. RNF213 also localizes on lipid droplets³¹ (also dependent on AAA-ATPase activity/ISG15), and is required for lysophosphatidic acid (LPA)-induced cell death in vitro³². Transfection studies implicate RNF213 in NF-κB signaling^{30,32}, and recently, the RNF213 RZ domain was found to ubiquitylate lipid A of Salmonella²⁹. In concert, these findings suggest roles for RNF213 in lipotoxicity and inflammation, both potentially relevant for MMD pathogenesis. The most highly penetrant MMD SNPs affect the RNF213 RING, not the RZ domain^{27,30,33}, leaving the detailed molecular pathogenesis of this disease—and its relationship, if any, to hypoxia hypersensitivity in PTP1B-deficient/inhibited breast cancer cells obscure.

In certain embodiments, inhibitors or activators of specific domains of the RNF213 protein may provide a therapeutic benefit to patients having one or more of breast cancer, pancreatic cancer, or other cancers with hypoxic tumor microenvironments (TMEs); to cancer patients treated with chemotherapy or targeted therapy that leave “drug-tolerant persister” cancer cells (DTPs), to individuals with Moyamoya Disease (MMD)/stroke/arthrosclerosis); non-alcoholic steatohepatitis (NASH), non-alcoholic fatty liver disease (NAFLD), or other types of lipotoxicity (e.g., primary lipodystrophies); autoimmune diseases and inflammatory diseases.

In certain embodiments, inhibitors or activators of specific domains of the RNF213 protein may provide a therapeutic benefit, e.g., in combination with a HER2 tyrosine kinase inhibitor (TKI) to treat a drug-tolerant population (DTP) in breast cancer and potentially other malignancies. In some embodiments, inhibiting expression or promoting degradation of RNF213 can have salutary effects on preventing DTP formation.

In some embodiments, an RNF213-targeted agent can be used to treat the above-listed conditions. For instance, for hypoxic breast tumors (and other hypoxic tumors), agents can include small molecule activators of RNF213 ATPase activity, RNF213 oligomerization, or the RNF213 RZ domain, or small molecule inhibitors of the RNF213 RING domain. In some embodiments, small molecule activators or inhibitors may be used in combination with conventional chemotherapy, antibody drug conjugates (ADCs), targeted therapies, therapeutic antibodies, or combinations thereof.

In another embodiment, for Moyamoya disease (MMD), stroke, or atherosclerosis, inhibitors of the ATPase or RZ domain of RNF213 or RNF213 proteolysis targeting chimeras (PROTACs) may have therapeutic potential in MMD. Inhibitors of specific tyrosine kinases (for ABL1/2-asciminib, dasatinib, imatinib, nilotinib; for TNK1, TP-5801) that promote RNF213 oligomerization can also have therapeutic potential. RNF213 interactors identified by TurboID proximity-based labeling described herein are involved in stroke, atherosclerosis, lipotoxicity, and cholesterol metabolism. A hallmark of strokes can be inflammatory cell death (pyroptosis). RNF213 triggers pyroptosis through activation of NF-κB in a stress condition when accompanied by hypoxia. Therefore, ABL or TNK inhibitors, inhibitors of the RNF213 ATPase domain or RZ domain, inhibitors of RNF213 oligomerization, RNF213 PROTACs, or activators of RNF213 RING domain could have utility in limiting neuronal cell death in strokes, MI, and other steno-occlusive disorders.

In yet another embodiment, for non-alcoholic steatohepatitis (NASH), non-alcoholic fatty liver disease (NAFLD), or lipotoxicity, RNF213 ATPase or RZ inhibitors, inhibitors of RNF213 oligomerization, RNF213 PROTACs, or activators of RNF213 RING domain may prevent or limit liver damage in NASH.

In certain embodiments, for autoimmune diseases and inflammatory diseases, wild-type RNF213 can promote NF-κB signaling through the degradation of CYLD/SPATA2. Impairing RNF213 ATPase activity or RZ activity, inhibiting RNF213 oligomerization, activators of RNF213 RING domain or inhibitors of tyrosine kinases for RNF213 (e.g., ABL1/2 or TNK1) could be therapeutic for such cases.

In another aspect, the present disclosure provides methods for preventing the generation of drug-tolerant persisters (DTPs), for example, in HER2+ breast cancer. RNF213 knockout cells are more susceptible to prescribed HER2+BC treatment drugs like lapatinib, tucatinib (HER2 inhibitors) suggesting that RNF213-KO cells are less likely to form DTPs. Combining a HER2-targeted agent with RNF213 PROTACs or inhibitors of other RNF213 domains could augment HER2-based therapies. ABL1/2 or TNK1 inhibition might also have benefits. In certain embodiments, for HER2+ breast cancer, combining a HER2-targeted agent with, e.g., RNF213 PROTACs, inhibiting RNF123 expression, or inhibitors or activators of specific RNF213 domains may be useful in the treatment of such cancers.

A tyrosine phosphorylation site in RNF213 was identified herein as being regulated by PTP1B (Y1275) and phosphorylated by ABL1/2. Y1275 phosphorylation promotes AAA-ATPase domain-dependent oligomerization of RNF213 and activation of its ubiquitin ligase activity. Proximity ligation experiments with Turbo-ID described herein identified multiple novel interacting proteins, including CYLD/SPATA2, a major negative regulator of NF-κB. Detailed analysis described herein shows that the RNF213, via its RZ domain, ubiquitylates CYLD/SPATA2 and, in concert with the LUBAC complex, leads to their degradation in hypoxia, NF-κB activation, induction of NLRP3, and inflammasome priming. The RZ domain also ubiquitylates Lipid A of Salmonella LPS, and in concert with LUBAC, promotes bacterial clearance by autophagy. Hypoxia-evoked ER stress then provides the “second signal” leading to inflammasome activation and Gasdermin D-dependent pyroptotic cell death. Hence, PTP1B inhibition/degradation, in addition to killing hypoxic tumor cells that are typically resistant to chemoradiation and targeted therapies, could “turn cold tumors hot” and facilitate immunological clearance. HER2+ breast cancer and many other breast cancer lines also show hypoxia hypersensitivity upon PTP1B inhibition. The RING domain of RNF213 negatively regulates RZ domain ubiquitin ligase activity and removes the requirement for the LUBAC complex to degrade CYLD/SPATA2 and activate NF-κB. The most common MMD SNPs affect the RING, and MMD alleles activate NF-κB. This may be related to the frequent co-occurrence of autoimmune and inflammatory diseases with MMD. Furthermore, several other interactors in the Turbo screen described herein are implicated in hypoxia and atherosclerosis such that RNF213 might be targeted for therapeutic benefit in those disorders.

As described herein, RNF213 promotes pyroptosis, a characteristic feature of vascular occlusive events. By understanding that tyrosine kinases ABL1/2 and TNK1 enhance RNF213 pyroptotic activity, use of tyrosine kinase inhibitors is explored for stroke treatment. Moreover, direct inhibitors targeting RNF213 are developed for greater specificity. A pathway and mechanism for targeting RNF213, a giant AAA-ATPase/ring finger-containing ubiquitin ligase is described, which promotes pyroptotic cell death in severe hypoxia. The role of RNF213 can require its RZ domain as well as its ATPase domain, whereas the RING domain inhibits the RZ domain. The ATPase activity of RNF213 controls oligomerization of RNF213 and is negatively regulated by the protein-tyrosine phosphatase PTP1B and positively regulated by ABL family kinases and TNK1. Single nucleotide polymorphism (SNPs) in RNF213 are known to be associated with Moyamoya disease. Inactivation of the RNF213 RING domain (where most of the highly penetrant MMD SNPs reside) promotes its RZ activity. Since this RZ activity is requires ATPase activity, the findings that RNF213 oligomerization is promoted by ABL family kinases and TNK1 could have therapeutic potential for Moyamoya disease, as well as for patients with more conventional occlusive events (e.g., stroke, myocardial infarction (MI), arterial thrombosis) and diseases of lipotoxicity (e.g., hepatic steatosis, lipodystrophy). Specifically, ABL, TNK1, or RNF213 RZ inhibitors or degraders could be active in these settings.

It was found that ABL1/2 phosphorylate, whereas PTP1B dephosphorylates, RNF213 on tyrosine 1275 (Y1275). Phosphorylation of this site promotes RNF213 oligomerization and activation of its E3 ligase activity. Via proximity-based proteomics, the CYLD-SPATA2 complex, a major negative regulator of NF-κB activation, was identified as an RNF213 substrate critical for hypoxia-induced cell death. Moreover, the RZ domain is required for CYLD-SPATA2 ubiquitylation/degradation, while the RING domain restrains RZ activity. Wild type RNF213 requires the LUBAC complex for effective CYLD-SPATA2 degradation, but RNF213 RING mutants, including highly penetrant SNPs associated with MMD, bypass this requirement. Increased RNF213 activity, caused by PTP1B deficiency/inhibition or RNF213 RING mutations, drives CYLD/SPATA2 ubiquitylation and degradation and increases NF-κB activity, priming cells for pyroptosis via the NLRP3 inflammasome. Concomitantly, hypoxia-induced ER stress delivers the “second signal” resulting in increased cancer cell death. The results described herein have implications for the mechanism of action and potential uses of dual inhibitors of PTP1B/TC-PTP (PTPN2)³⁴, now in clinical trials for various cancers, as well as for the pathogenesis of MMD and other steno-occlusive disorders.

### Inhibitors of RNF213 Protein Expression

In some embodiments, the inhibitor of RNF213 protein expression is an siRNA, an shRNA, an antisense oligonucleotide, a miRNA, or a site-specific nuclease. Non-limiting examples of the inhibitor are described below.

In some embodiments, the inhibitor is a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e., a ssRNA molecule comprising just an antisense strand) or double stranded (i.e., a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 more nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprise an antisense strand having a nucleotide sequence that has up 0, 1, 2, or 3 mismatches over 15-22 bases with a target sequence. In some embodiments, siRNA molecules comprise an antisense strand having a nucleotide sequence that has 0, 1, or 2 mismatches over 15-22 bases with a target sequence. In some embodiments, siRNA molecules comprise an antisense strand having a nucleotide sequence that has 0 or 1 mismatch over 15-22 bases with a target sequence. In some embodiments, siRNA molecules comprise an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated in the present disclosure. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In some embodiments, the inhibitor is a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

In some embodiments, the inhibitor of RNF213 protein expression comprises a site-specific nuclease. In some embodiments, the site-specific nuclease comprises a DNA nuclease such as an engineered (e.g., programmable or targetable) DNA nuclease to induce genome editing of a target DNA sequence of RNF213. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof.

In some embodiments, the inhibitor of RNF213 protein expression is an antisense oligonucleotide (ASO). An ASO can downregulate a target, for example, by steric hindrance of ribosomal activity, by causing RNase H endonuclease cleavage of a target RNA, or by altering splicing or by inhibiting 5′ cap formation. An ASO can generally comprise a short nucleotide sequence which is substantially complementary to a target nucleotide sequence in a pre-mRNA molecule, an mRNA molecule, or a heterogeneous nuclear RNA (hnRNA). The degree of complementarity (or substantial complementarity) of the antisense sequence can be such that a molecule comprising the antisense sequence may form a stable double-stranded hybrid with the target nucleotide sequence in the RNA molecule. Without wishing to be bound by theory, “complementarity” of nucleic acids can mean that a nucleotide sequence in one strand of nucleic acid, e.g., due to orientation of its nucleobase groups, forms hydrogen bonds with another sequence on an opposing nucleic acid strand. The complementary bases in DNA are generally A paired with T and C paired with G. In RNA, the complementary bases are generally C with paired with G and U paired with A. Complementarity can be perfect or substantial/sufficient. Perfect complementarity between two nucleic acids means that the two nucleic acids can form a duplex in which every base within the duplex is bonded to a complementary base by Watson-Crick pairing. “Substantial” or “sufficient” complementarity means that a sequence in one strand is not completely and/or perfectly complementary to a sequence in an opposing strand but that sufficient bonding takes place between bases on the two strands to form a stable hybrid complex in set of hybridization conditions (e.g., temperature and/or salt concentration). Such conditions can be determined by, e.g., empirical determination of Tₘ (melting temperature) by employing routine methods in the art or by using the sequences and standard mathematical calculations to predict the Tₘ of hybridized strands. Tₘ can include the temperature at which a population of hybridization complexes formed between two nucleic acid strands are 50% denatured (i.e., a population of double-stranded nucleic acid molecules becomes half dissociated into single strands). At a temperature below the Tₘ, formation of a hybridization complex can be favored, while at a temperature above the Tₘ, melting or separation of the strands in the hybridization complex can be favored.

In some embodiments, an ASO is a morpholino or a gapmer.

Antisense oligonucleotides can be synthetic and chemically modified.

In some embodiments, antisense oligonucleotides may be 100% complementary to the target sequence, or may comprise mismatches. so long as a heteroduplex formed between the oligonucleotide and the target sequence is sufficiently stable to tolerate the action of modes of degradation which may occur in vivo, e.g., by way of cellular nucleases. Mismatches, if present, are generally less destabilizing toward the end regions of the hybrid duplex than in the middle. The number of mismatches permitted can depend on, e.g., the percentage of G:C base pairs in the duplex, the length of the oligonucleotide, and/or the position of the mismatch(es) in the duplex, according to principles of duplex stability within the knowledge of one skilled in the art. In some embodiments, an oligonucleotide may have about 70% to about 100% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

### RNF213 Protein Degraders

In some embodiments, degrading RNF213 protein comprises administering to the subject an effective amount of a proteolysis targeting chimera (PROTAC) or a molecular glue.

Proteolysis targeting chimeras (PROTACs) are bifunctional molecules that combine a ligand for an E3 ligase with a second ligand that targets a protein of interest and catalyze its polyubiquitination and eventual proteasomal degradation. Both ends of the PROTACs are connected via a linker. First generation PROTACs used peptides to recruit a protein of interest to E3 ligases, but subsequent ones have relied on smaller and more cell-permeable synthetic ligands. These include hydroxyproline derivatives and molecules derived from thalidomide, which bind the von Hippel-Lindau protein (VHL) and cereblon (CRBN), respectively. VHL and CRBN are the substrate receptors of two cullin-RING ubiquitin ligase (CRL) complexes, namely CRL2^{VHL} and CRL4^{CRBN}.

Molecular glues are small molecule protein degraders that are capable of inducing interactions between an E3 ubiquitin ligase and a protein, thereby resulting in ubiquitination and degradation of the protein. Without wishing to be bound by theory, upon binding to a protein, a molecular glue can induce a conformational change and render the small molecule-protein complex a “neo-substrate” for E3 ligase. Following the formation of a ternary complex, the “neo-substrate” is ubiquitinated, which leads to ubiquitin-proteasome system (UPS)-mediated protein degradation. Non-limiting examples of molecular glues are anti-cancer aryl-sulfonamides, e.g., Indisulam and CR-83 (a CDK inhibitor). In some embodiments, a molecular glue can drive protein degradation by making novel interactions between ubiquitin ligases and neo-substrates.

### EXAMPLES

The following examples are provided to further describe some of the embodiments disclosed herein. The examples are intended to illustrate, not to limit, the disclosed embodiments.

### Example 1. Y1275 Phosphorylation of RNF213 is Regulated by PTP1B/ABL, which Promotes RNF213 Oligomerization

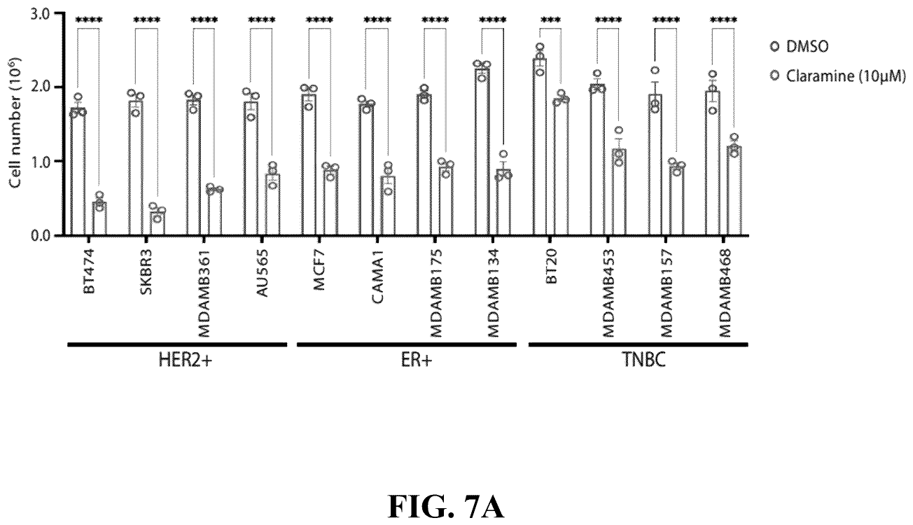

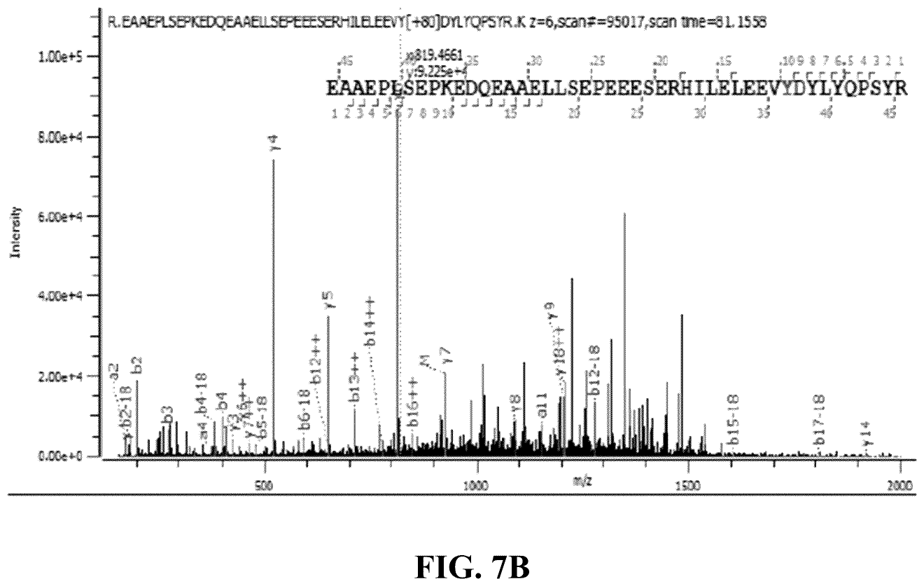

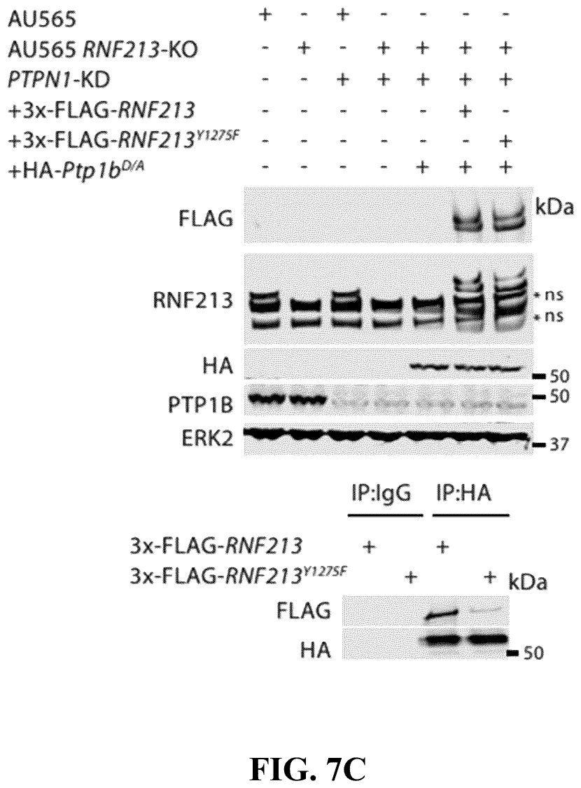

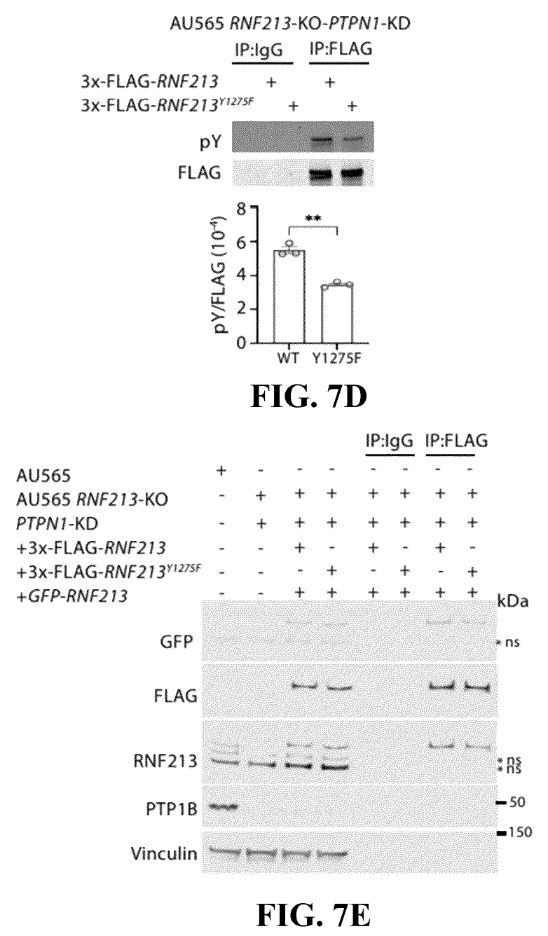

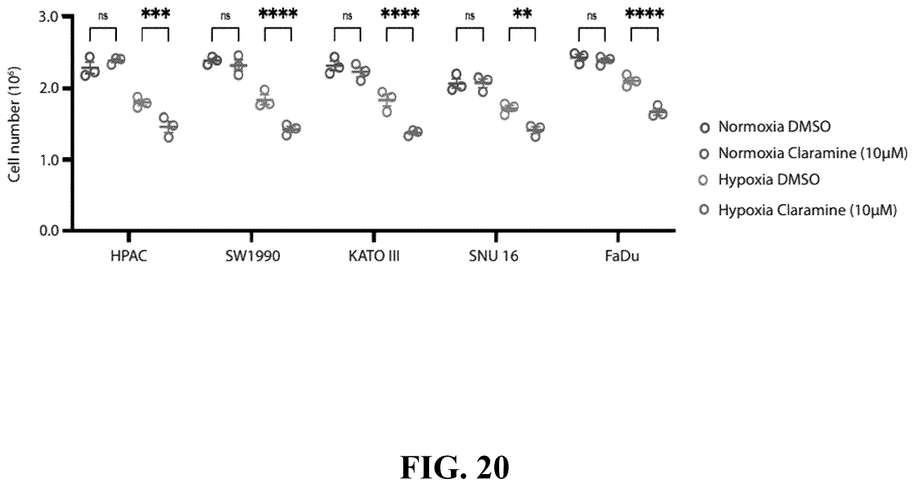

HER2+ breast cancer cells with PTPN1 knockdown or treated with a PTP1B inhibitor are hypersensitive to hypoxia²². However, by studying an expanded panel of breast cancer cell lines, hypoxia hypersensitivity is not limited to the HER2+ subtype; treatment with the allosteric PTP1B inhibitor claramine also caused hypoxia hypersensitivity in several ER+ and triple negative breast cancer (TNBC) lines (FIG. 7A, FIG. 20). Hence, PTP1B is more general regulator of the hypoxia response, at least in breast cancer cells and possibly in other malignancies.

RNF213 is basally tyrosine phosphorylated in HER2+ breast cancer cells and interacts with PTP1B. “Substrate-trapping” mutants of PTP1B show increased RNF213 binding, and this interaction is competed by the active site PTP inhibitor sodium orthovanadate, suggesting that RNF213 is a PTP1B substrate²². However, RNF213 tyrosine phosphorylation is not increased in PTP1B-inhibited/deficient HER2+ breast cancer cells, indicating that only select sites are PTP1B targets. An RNF213-TurboID fusion protein was generated with the goal of identifying RNF213 binding partners by proximity ligation³⁵, labelled interacting proteins with biotin, enriched these proteins on streptavidin beads, performed on-bead tryptic digestion, and analyzed the peptide mixture by mass spectrometry (MS). This analysis also revealed evidence of RNF213 phosphorylation on residue Y1275 (FIG. 7B and Tables 7-9).

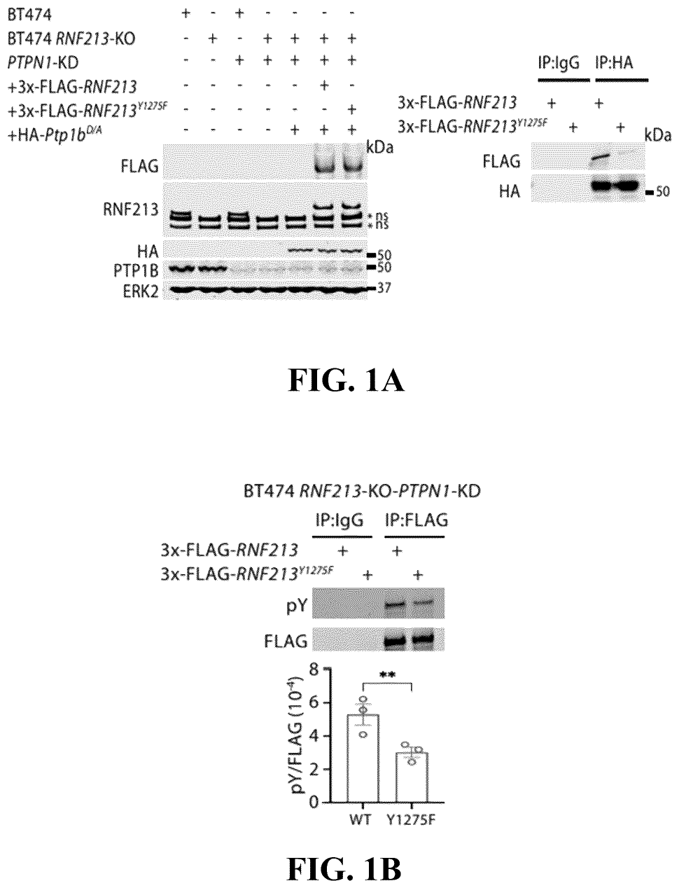

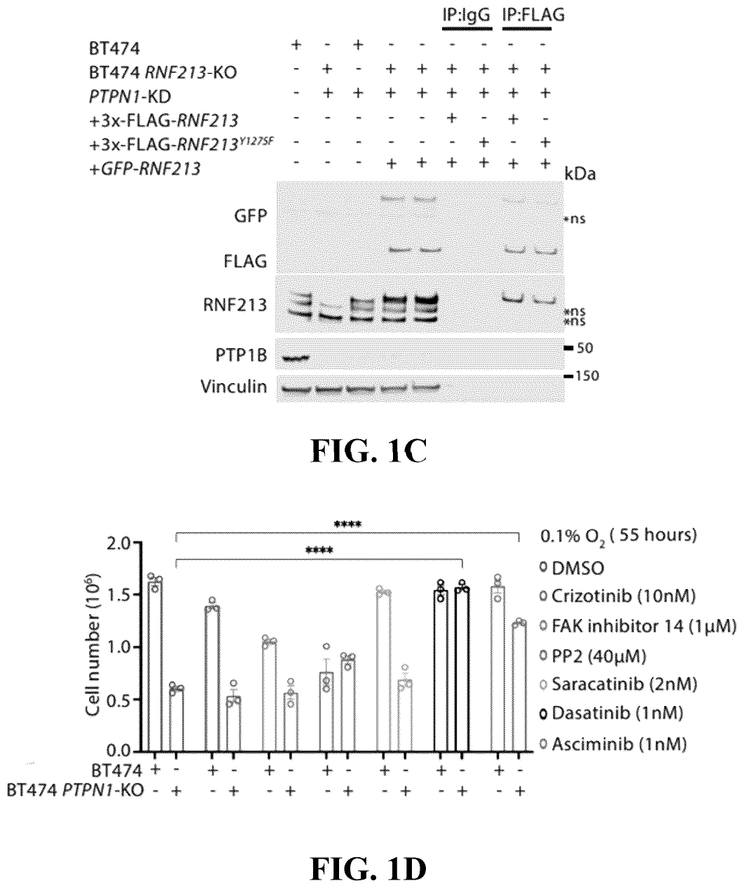

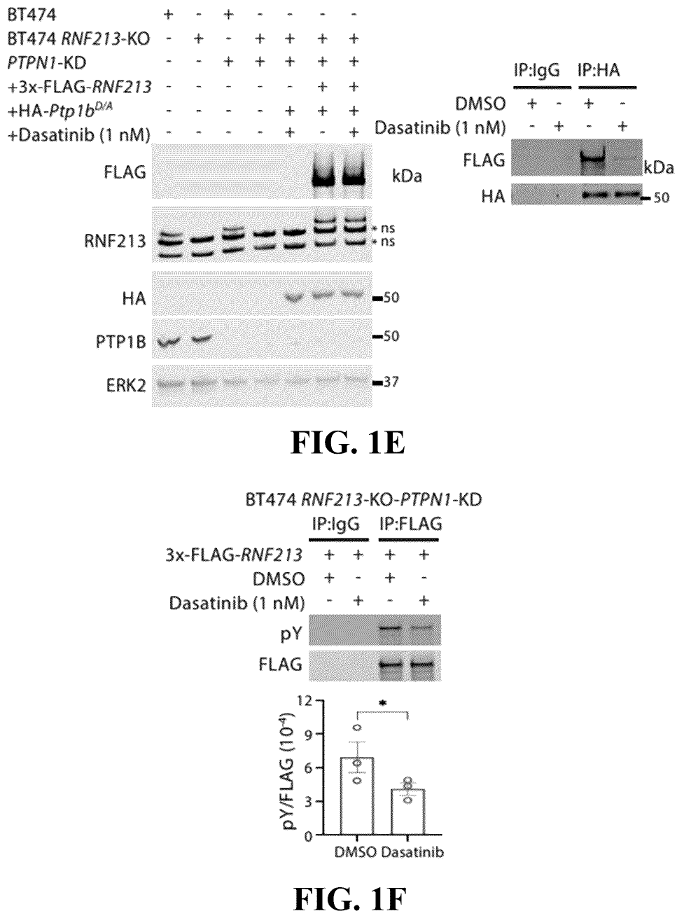

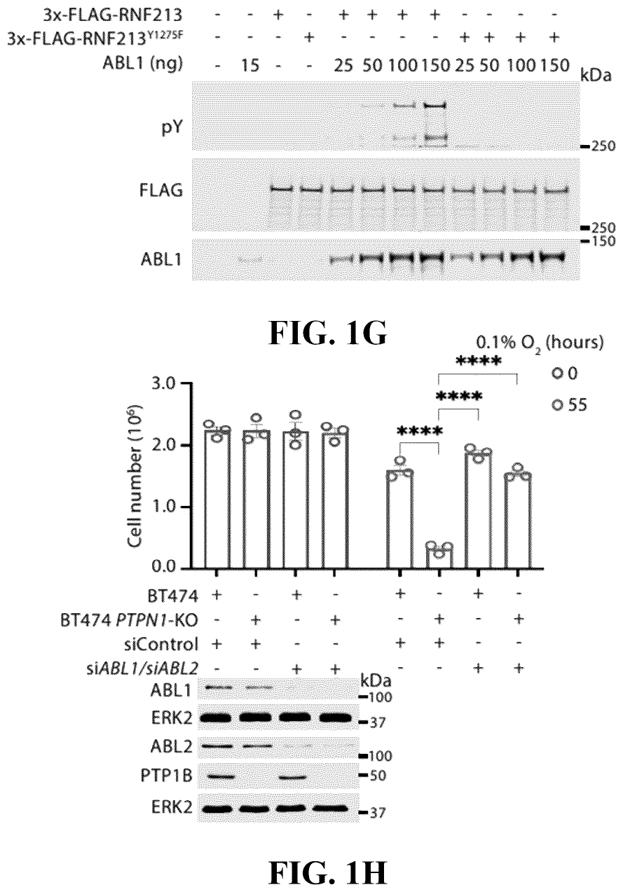

CRISPR/Cas9 was used to generate RNF213-knockout (KO) BT474 cells and retroviral gene transduction to introduce PTPN1 or non-targeting (Control) shRNAs. This verified that phosphorylation was relevant for RNF213/PTP1B interaction. The cells were transduced with a lentivirus expressing hemagglutinin (HA) tagged-mouse Ptpn1^{D/A}, which encodes the substrate-trapping mutant PTP1B^{D181A} (HA-PTP1B^{D/A}). The resultant lines were transfected with expression vectors for 3×-FLAG-RNF213 or 3×-FLAG-RNF213^{Y1275F}. Immunoprecipitation (IP) of HA-PTP1B^{D/A} resulted in recovery of 3×-FLAG-RNF213, while recovery of 3×-FLAG-RNF213^{Y1275F} was reduced markedly (FIG. 1A, right panel), even though it was expressed at comparable levels to wild-type RNF213 (FIG. 1A, left panel). Similar results were obtained from analogous experiments with AU565-derived cell lines (FIG. 7C). Tyrosine phosphorylation of 3×-FLAG-RNF213^{Y1275F} was decreased compared with that of 3×-FLAG-RNF213, but to a lesser extent (˜40%) than the near total decrease in its recovery in HA-mPTP1B^{D/A} immunoprecipitates (FIG. 1B and FIG. 7D). These data suggest that additional tyrosine phosphorylation sites exist in RNF213 that are not detected by MS or that new sites are generated in RNF213^{Y1275F}. Regardless, the results disclosed herein indicate that RNF213-Y1275 is a specific site for PTP1B-catalyzed dephosphorylation.

PTP1B deficiency/inhibition markedly increases overall cellular ubiquitylation, dependent on RNF213²². Yet Y1275 is distant from the RING or RZ domains in the monomeric RNF213 structure²⁶, arguing against direct regulation. The hypothesis that phosphorylation affects RNF213 oligomerization was tested by co-transfecting expression vectors for 3×-FLAG-RNF213 or 3×-FLAG-RNF213^{Y1275F} and GFP-RNF213 and performing anti-FLAG immunoprecipitations. Notably, recovery of GFP-RNF213 in 3×-FLAG-RNF213^{Y1275F} immunoprecipitates was impaired in BT474 (FIG. 1C) and AU565 cells (FIG. 7E).

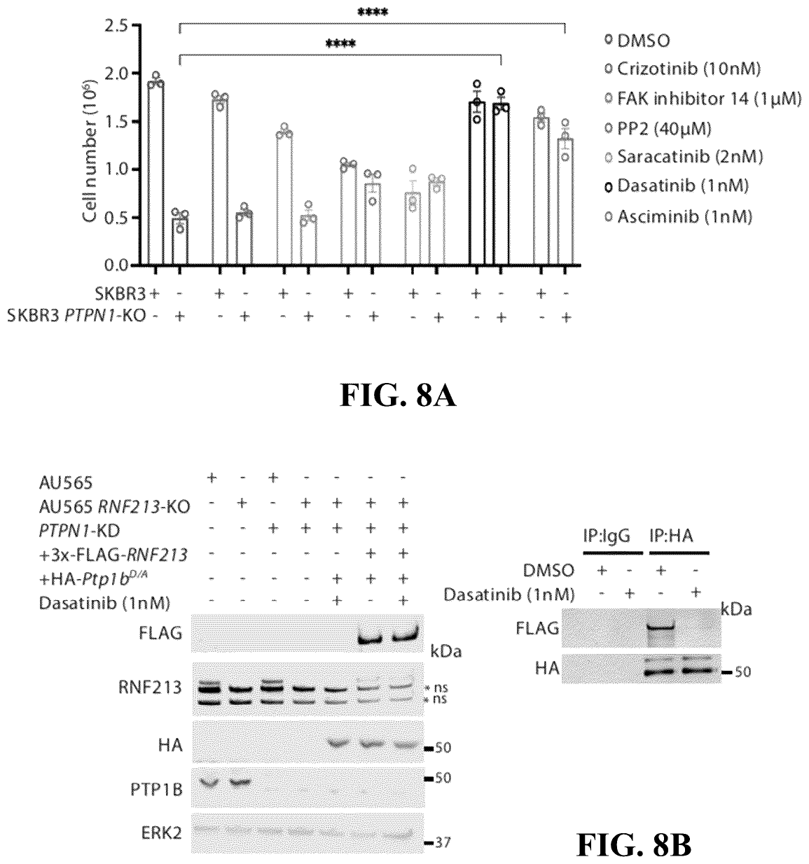

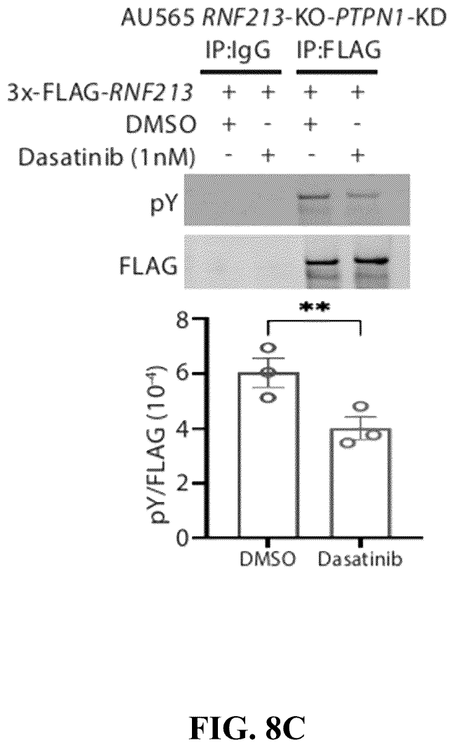

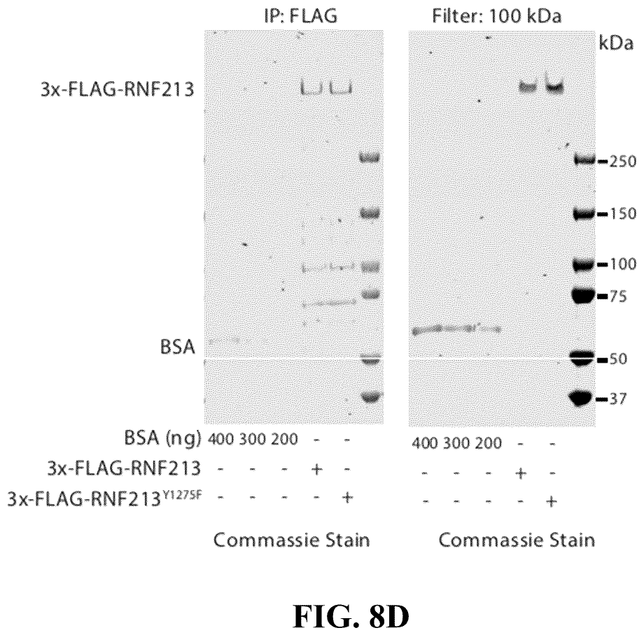

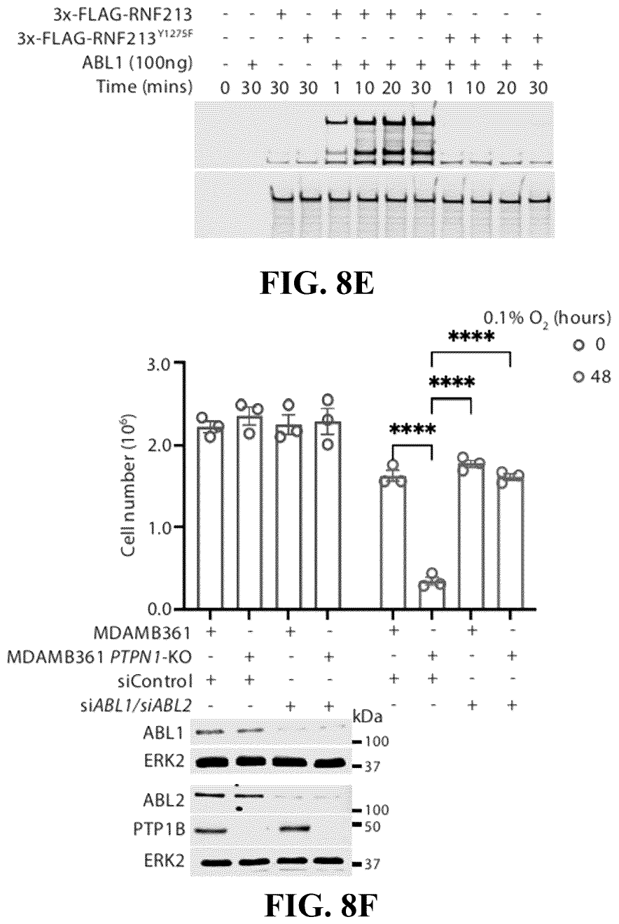

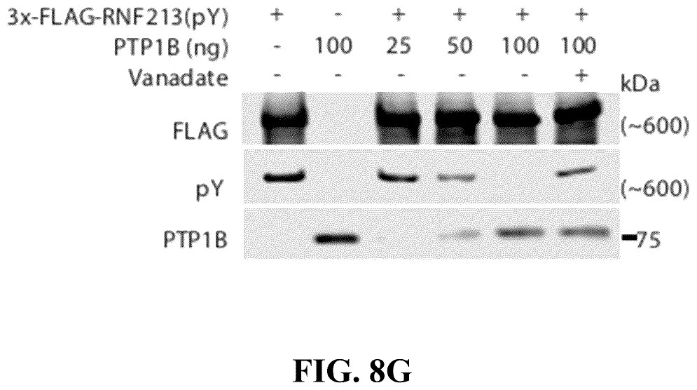

The kinase(s) that phosphorylate(s) RNF213-Y1275 were investigated. ABL family kinase inhibitors (dasatinib, asciminib) abrogated the hypoxia hypersensitivity of BT474 PTPN1-KO cells and SKBR3 PTPN1-KO cells (FIG. 1D and FIG. 8A). Dasatanib treatment (compared to treatment with DMSO vehicle alone) also dramatically reduced the recovery of RNF213 in PTP1B trapping mutant immunoprecipates from these cells (FIG. 1E, right panel and FIG. 8B) and reduced overall RNF213 tyrosine phosphorylation to an extent similar to that caused by Y1275F mutation (FIG. 1F and FIG. 8C). Whether ABL1 phosphorylates RNF213 in vitro was evaluated. Immunopurified FLAG IPed 3×-FLAG-RNF213 and 3×-FLAG-RNF213^{Y1275F} (FIG. 8D) were incubated with recombinant His-tagged ABL1. Indeed, His-ABL1 phosphorylated WT-RNF213, but not RNF213^{Y1275F} in a time- and ABL1-concentration-dependent manner (FIG. 1G and FIG. 8E). Conversely, GST-PTP1B dephosphorylated ABL1-phosphorylated RNF213 (FIG. 8G). Furthermore, ABL1/2 siRNA-treated cells were resistant to hypoxia hypersensitivity induced by PTP1B deficiency (FIG. 1H). Similar results were observed using parental and PTPN1-KO MDA-MB-361 cells (FIG. 8F). It was determined that RNF213 Y1275 phosphorylation is regulated by PTP1B and ABL1/2, and phosphorylation of this site promotes RNF213 oligomerization and hypoxia hypersensitivity.

### Example 2. RNF213 Interacts with CYLD and SPATA2

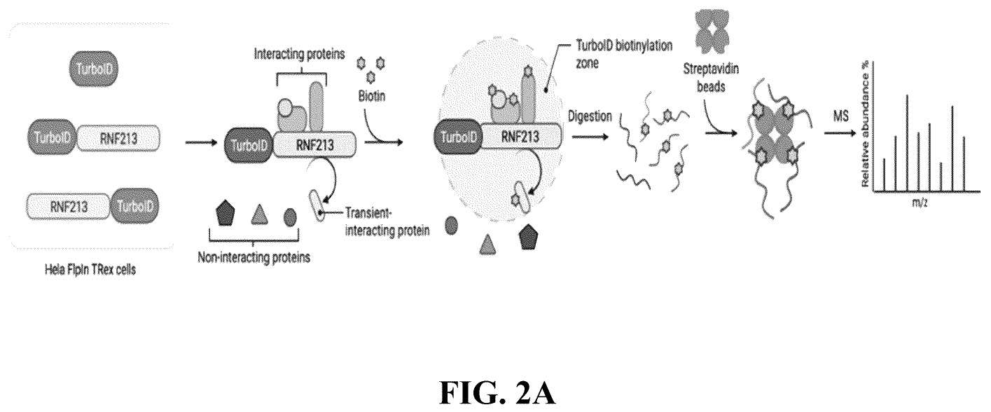

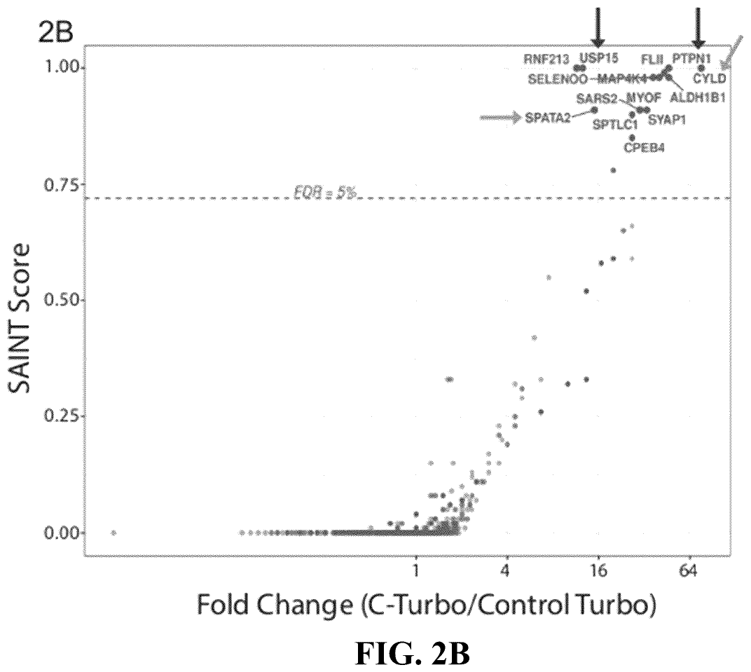

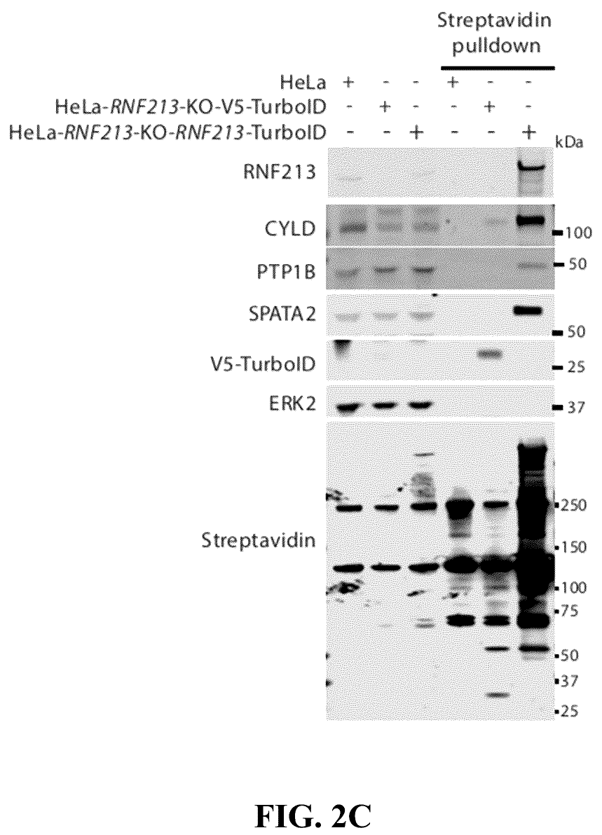

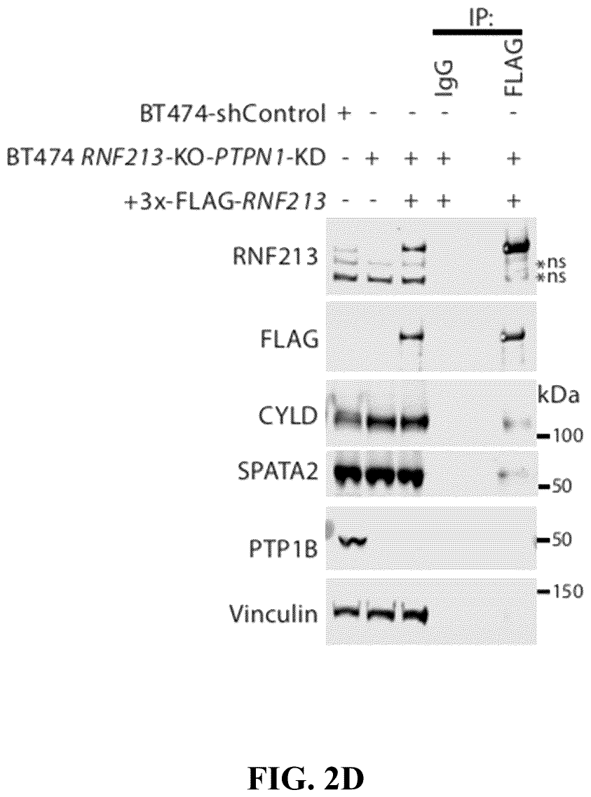

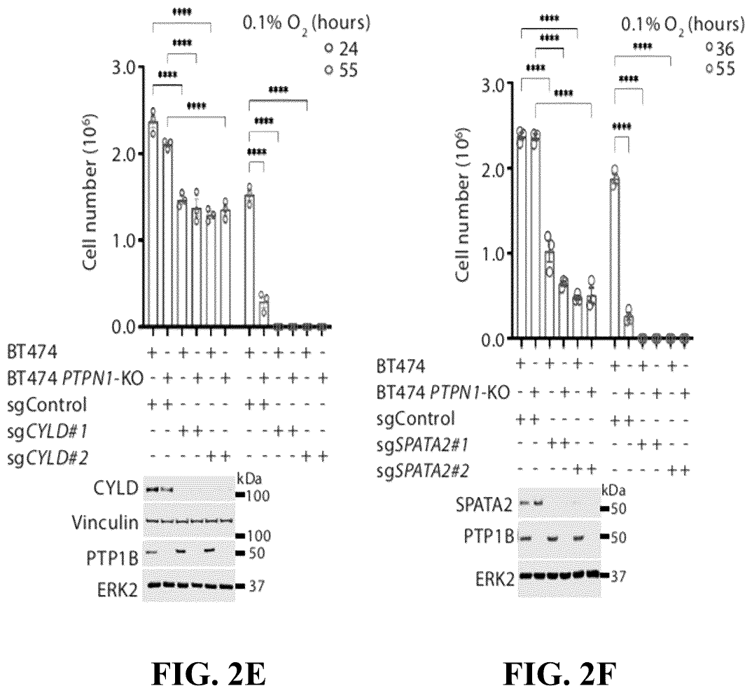

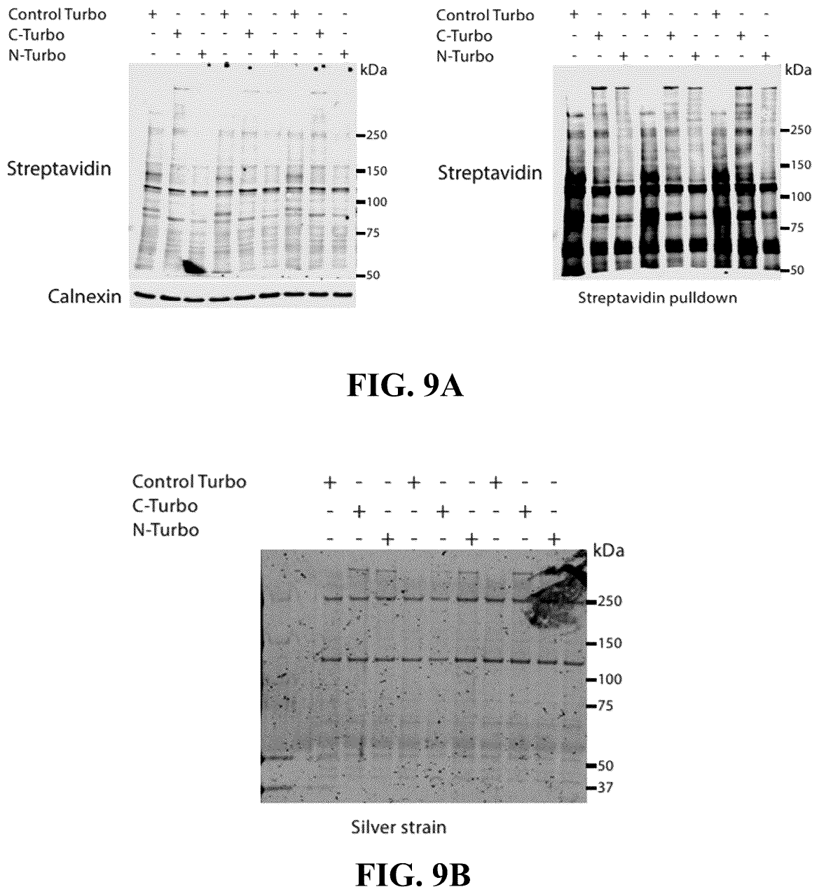

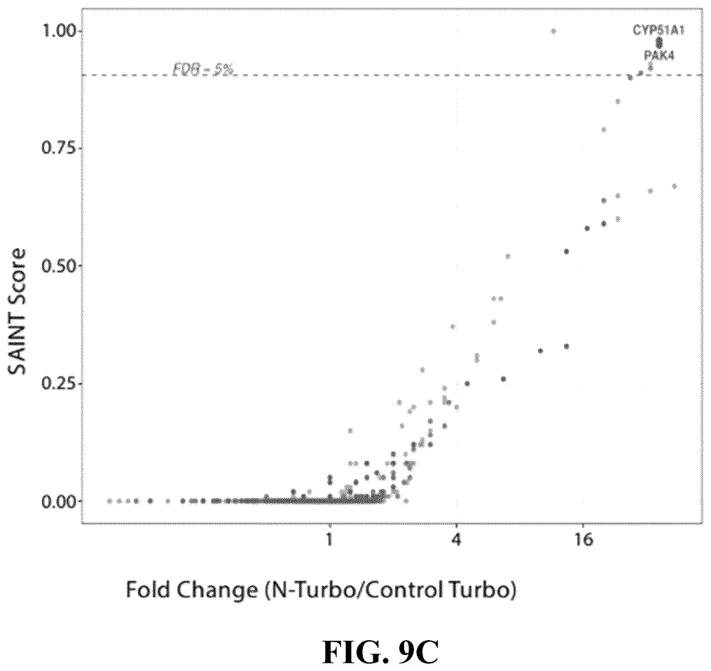

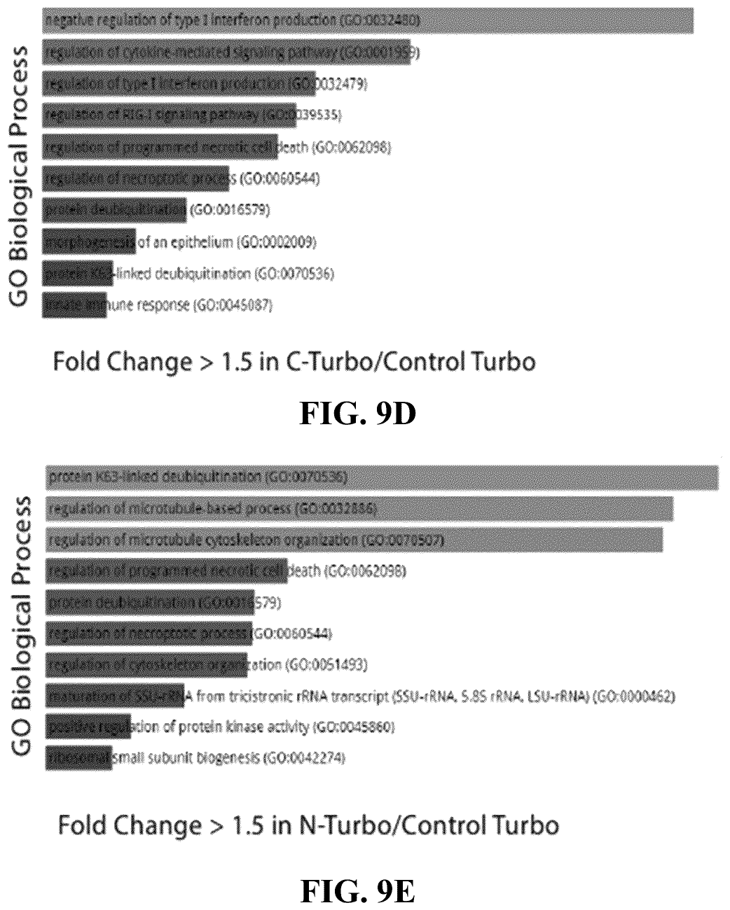

RNF213-interacting proteins were identified to evaluate how RNF213 regulates hypoxia sensitivity (FIG. 2A). TurboID-RNF213 (“N-Turbo”), RNF213-TurboID (“C-Turbo”), or TurboID alone (Control Turbo) were recombined into HeLa FlpIn-TRex RNF213-KO cells. These cells were labelled with biotin for 10 min., and biotinylated proteins were recovered on streptavidin, digested with trypsin, and analyzed by MS (FIG. 2A). Proteins were ranked as potential binding partners by using the SAINT express algorithm³⁶ Immunoblotting (FIG. 9A) and silver staining (FIG. 9B) confirmed that similar amounts of each sample were submitted for analysis. Relatively few proteins labelled by N-Turbo were labelled to an extent greater than their labeling by Control-Turbo (FIG. 9C). By contrast, in the C-Turbo experiment, PTP1B (PTPN1) and USP15 (blue-colored arrows), previously reported to interact with RNF213^{22,37}, as well as several new proteins were significantly enriched over the control sample (FIG. 2B and Tables 10-12). Among the other top candidates were the major K63-deubiquitinase and NF-κB regulator CYLD and its interacting protein SPATA2 (FIG. 2B, orange-colored arrows). Notably, RNF213/CYLD interaction was recently reported, although its biological significance was not determined³⁸. Other top C-Turbo interactors included proteins involved in VEGF signaling, which is induced in hypoxia (FLII, MYOF^{39,40}), inflammation and atherosclerosis (MAP4K4, SPTLC1^{41,42}), lipotoxicity (SPTLC1, ALDH1B1^{43,44}), ischemia response (CPEB4⁴⁵), and stroke (SARS2⁴⁶). Top N-Turbo interactors included CYP51A1, which plays a crucial role in cholesterol metabolism and atherosclerosis⁴⁷. Pathway analysis suggests involvement of RNF213 in K63-linked deubiquitylation, cytokine (including interferon)-mediated signaling, innate immunity, necroptotic cell death, and cytoskeletal regulation (FIGS. 9D and 9E).

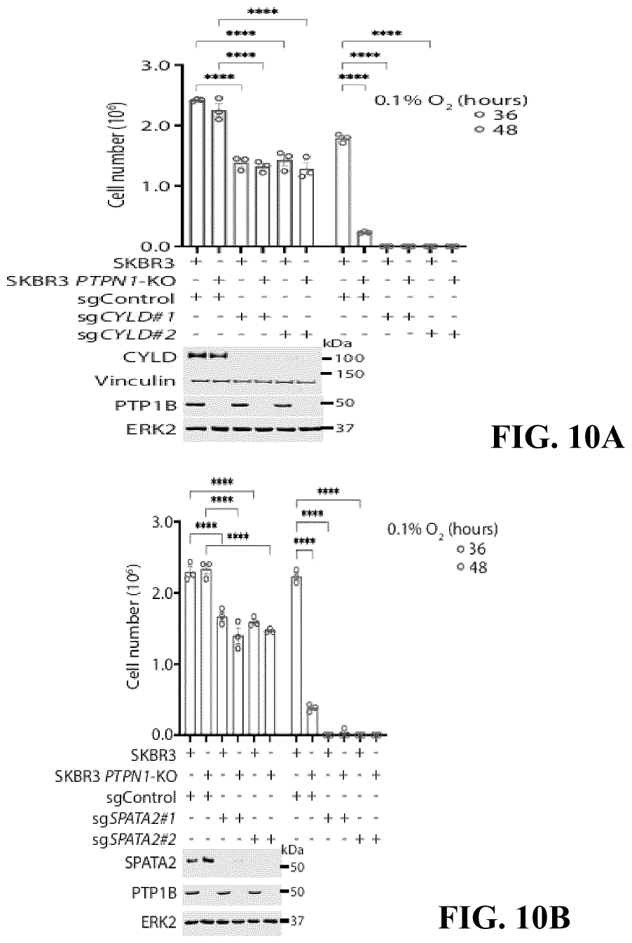

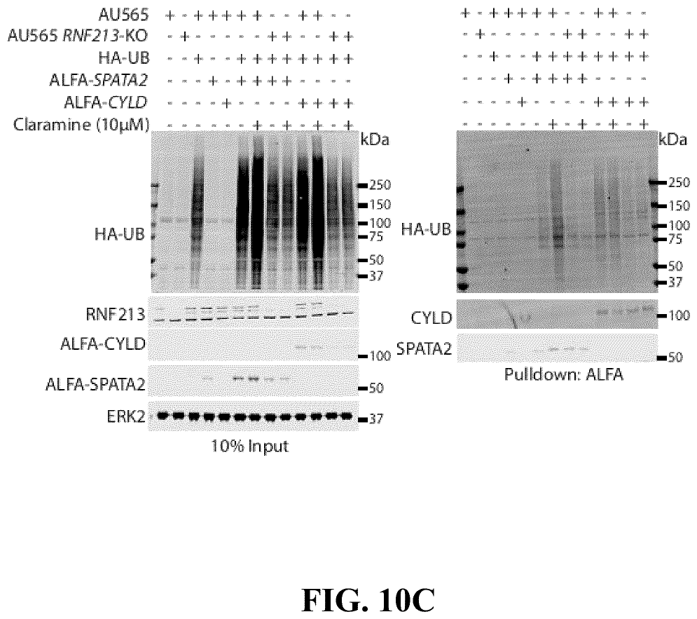

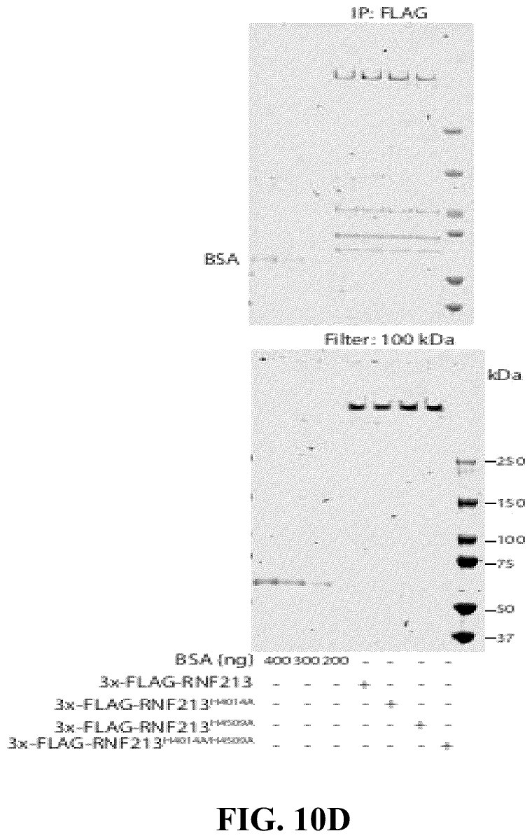

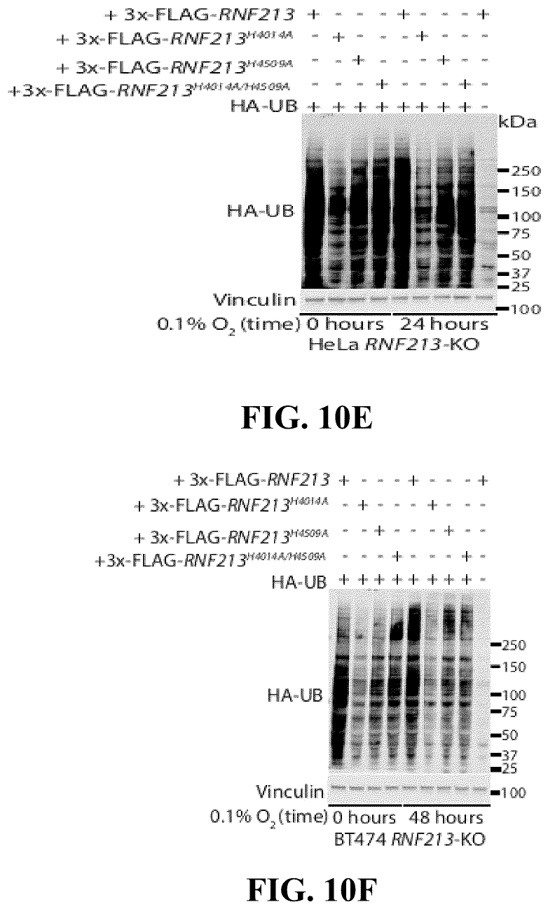

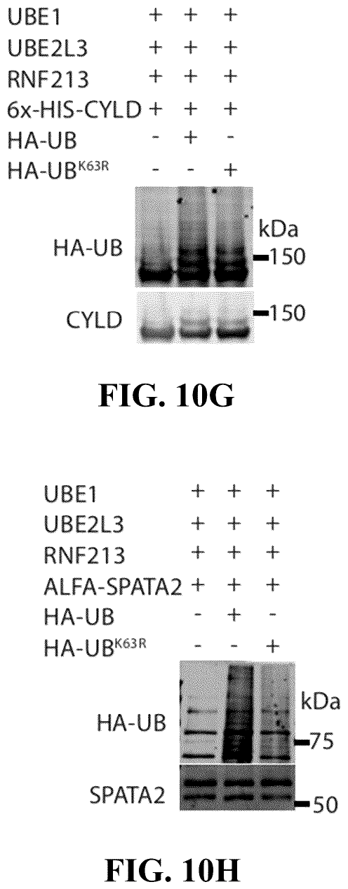

Given their role in NF-κB response, inflammation, and innate immunity, the CYLD/SPATA2 interaction with RNF213 was evaluated. Immunoblotting confirmed the interaction of C-Turbo, but not Turbo alone, with CYLD, SPATA2, and PTP1B; neither Turbo protein interacted with ERK2, which served as a control for non-specific biotinylation (FIG. 2C). CYLD and SPATA2 also co-immunoprecipitated with 3×-FLAG-RNF213 expressed by transient transfection of BT474-RNF213-KO-shPTPN1 cells (FIG. 2D). CRISPR/Cas9 technology and two independent sgRNAs were used to delete each gene in BT474 and BT474 PTPN1-KO cells. Remarkably, CYLD-KO or SPATA2-KO sensitized BT474 cells to hypoxia even more than PTPN1-KO alone (FIG. 2E). Similar results were obtained when analogous SKBR3-derived cell lines were studied (FIGS. 10A and 10B). Hence, RNF213 interacts with CYLD and SPATA2, and CYLD and SPATA2 (and, most likely, the CYLD/SPATA2 complex) prevent hypoxia hypersensitivity in breast cancer cell lines.

### Example 3. RNF213 Uses its RZ Domain to Ubiquitylate CYLD and SPATA2

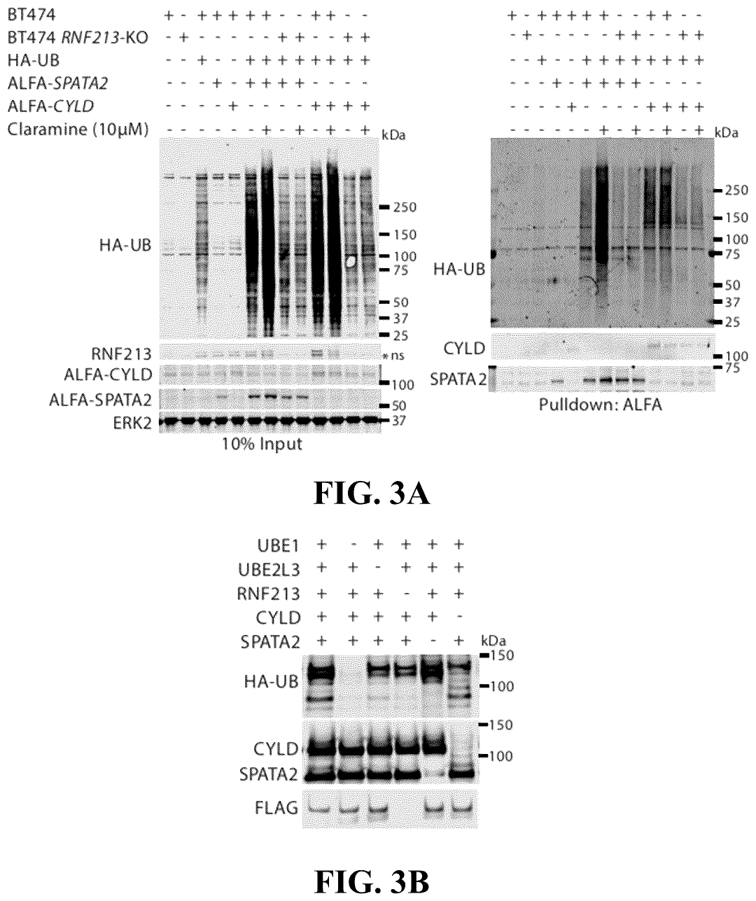

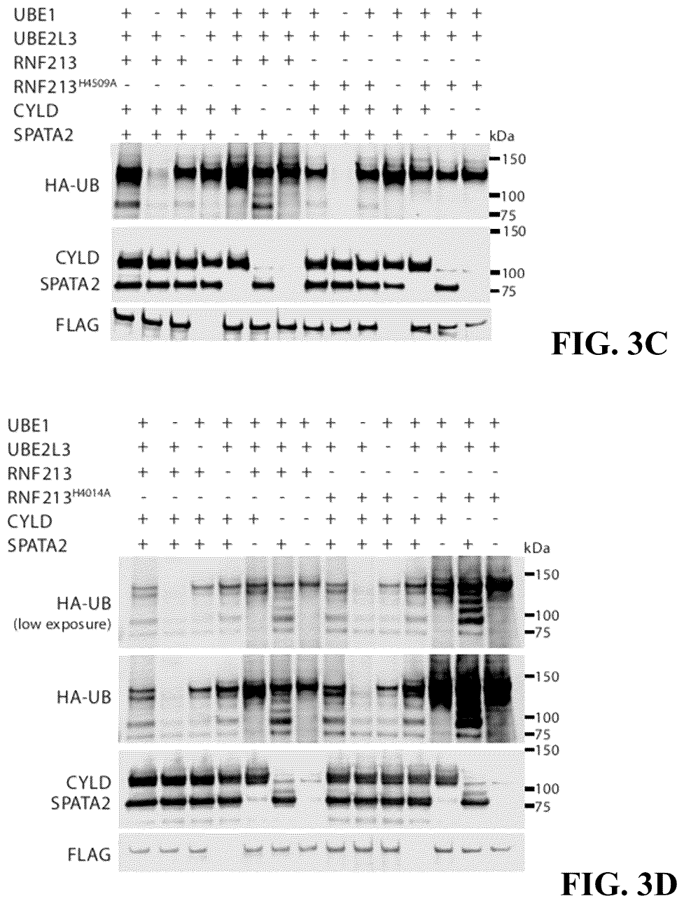

ALFA-tagged SPATA2 or CYLD and HA-Ubiquitin (HA-UB) in parental or RNF213-KO BT474 cells were co expressed to test whether CYLD and SPATA2 are RNF213 substrates. The cells were treated with claramine (10 μM) or vehicle (FIG. 3A). Co-transfection of UB resulted in an increase in HA-reactive species, which were enhanced further in ALFA-CYLD- or ALFA-SPATA2-expressing cells. Comporting with prior studies²², claramine treatment led to an increase in overall ubiquitylation (FIG. 3A, left panel). Recovery of each ALFA-tagged protein, followed by anti-HA immunoblotting, revealed RNF213-dependent ubiquitylation of CYLD and SPATA2, which was enhanced by PTP1B inhibition (FIG. 3A, right panel). Analogous results were obtained using AU565 cells (FIG. 10C).

3×FLAG-tagged RNF213 and various RNF213 mutants were purified from HeLa FlpIn-TRex RNF213-KO cells (FIG. 10D top, bottom), and assessed the ability of each protein to ubiquitylate CYLD and SPATA2 in vitro using UBE1 as the E1 enzyme and UBE2L3 as the E2. Consistent with the transfection studies, SPATA2 and CYLD, when tested alone or in equimolar concentrations, were ubiquitinated by 3×FLAG-RNF213 (FIG. 3B). As noted above, RNF213 has two potential ubiquitin ligase domains (RING, RZ). There was no significant ubiquitylation of CYLD or SPATA2 when an RZ mutant (“RZ-dead”) form of RNF213 (RNF213^{H4509A}) was tested (FIG. 3D). Surprisingly, however, both proteins showed increased ubiquitylation when the RING mutant RNF213^{H4014A} (RING-dead) was assayed (compared with WT RNF213). Based on these results, it was hypothesized that the RING negatively regulates the RZ domain. The ubiquitylation activity of WT RNF213 was compared to RNF213^{H4014A/H4509A} (RING/RZ-dead); however, RING/RZ-dead was unable to ubiquitylate CYLD/SPATA2 (FIG. 3E). Therefore, RNF213 is an E3-ubiquitin ligase for CYLD and SPATA2, their ubiquitylation is catalyzed by the RZ domain, and the RING can restrain RZ activity. CYLD and SPATA2 ubiquitylation were reduced (but not eliminated) when K63R-UB was used as the substrate (FIGS. 10G and 10H). Therefore, RNF213 catalyzes CYLD and SPATA2 ubiquitylation; the RZ catalyzes ubiquitylation with a preference for K63 of UB; and the RING restrains RZ activity. Furthermore, RING-dead (or RING-impaired) mutations are RNF213 RZ gain-of-function mutants; notably, highly penetrant MMD SNPs map to the RING domain.

It was reported that RING-dead RNF213 or highly penetrant MMD SNPs decrease global ubiquitylation in HeLa or 293T cells and appear to act as dominant negative alleles. By contrast, the disclosure provided herein suggests that RING-dead RNF123 is an RZ gain-of-function mutation, which might be expected to increase global ubiquitylation. In an attempt to resolve this apparent discrepancy, 3×FLAG-RNF213 or RNF213 mutants were co-expressed with HA-Ubiquitin (HA-UB) in HeLa Flp-In-TRex RNF213-KO cells (FIG. 10E) and subjected these cells to normoxia or hypoxia (0.1% O₂ for 24 hours). A sharp decrease in global ubiquitylation with the RING-dead mutant was observed; however, global ubiquitylation also was decreased in cells expressing RZ-dead or RING- and RZ-dead RNF213. Similar results were observed in experiments with BT474 RNF213-KO cells reconstituted with RNF213 or various RNF213 mutants (FIG. 10F). These data suggest that RING and RZ domain each have unique substrates, and that negative regulation of the RZ domain by the RING is probably substrate specific. Alternatively, the RZ domain could regulate targets that, in concert, perturb global ubiquitylation.

### Example 4. The RNF213 RZ and ATPase Domains Promote Hypoxia Hypersensitivity Via Degradation of SPATA2 and CYLD

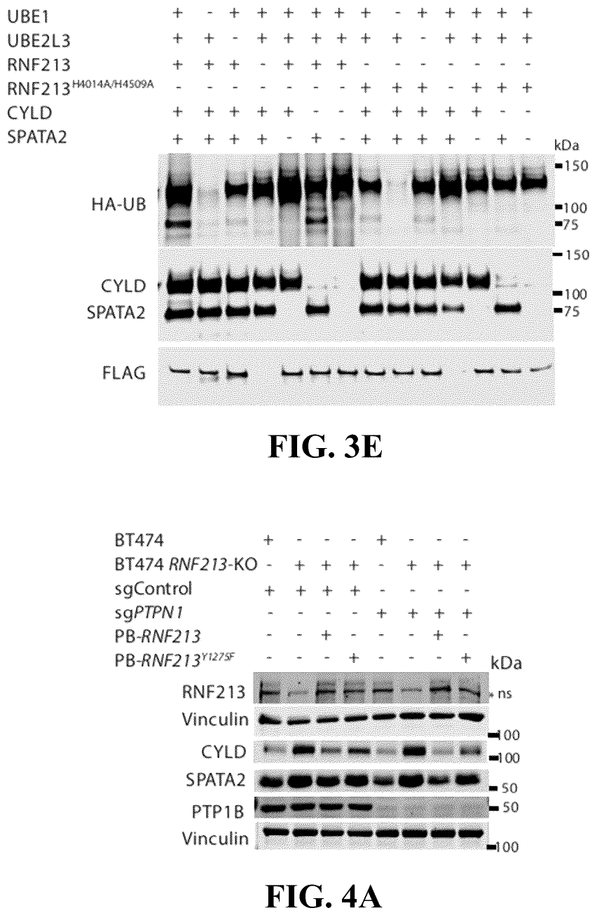

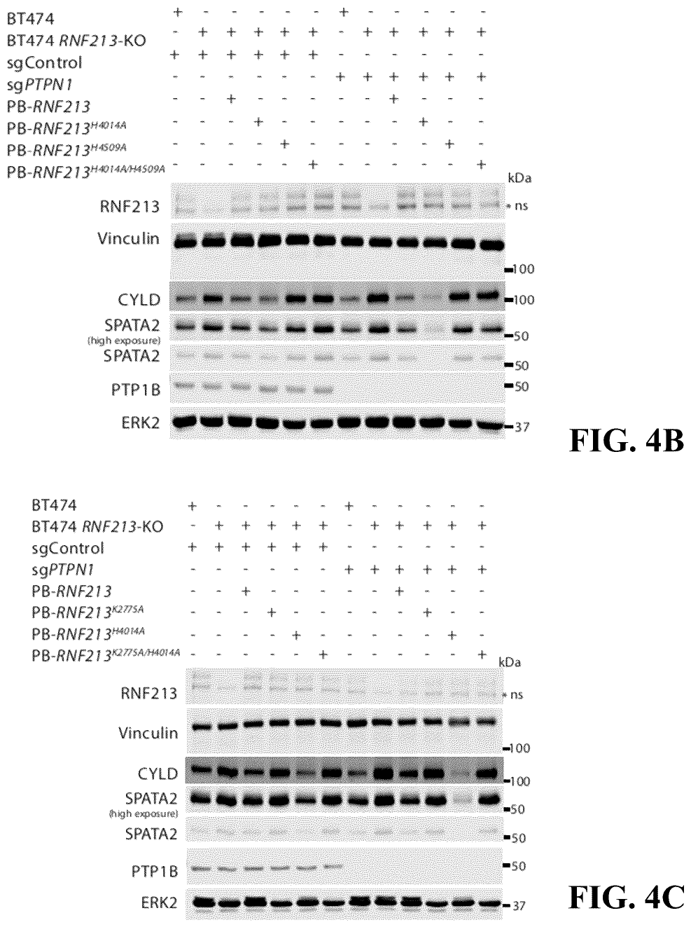

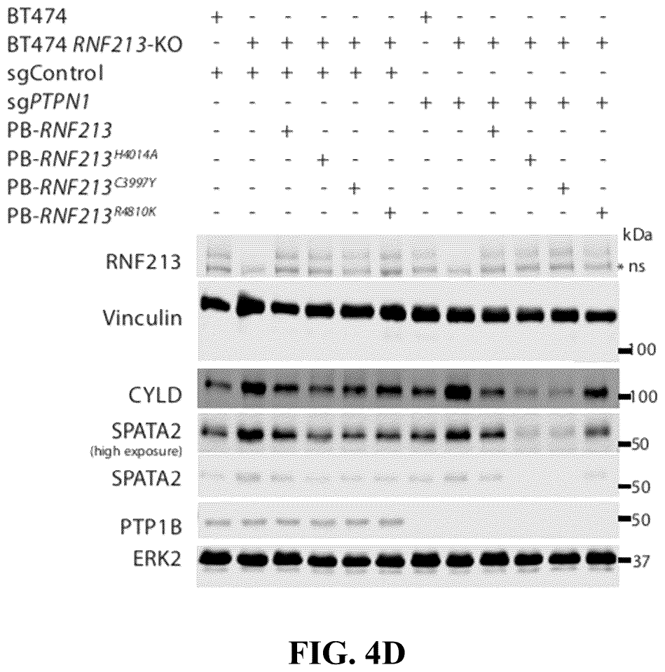

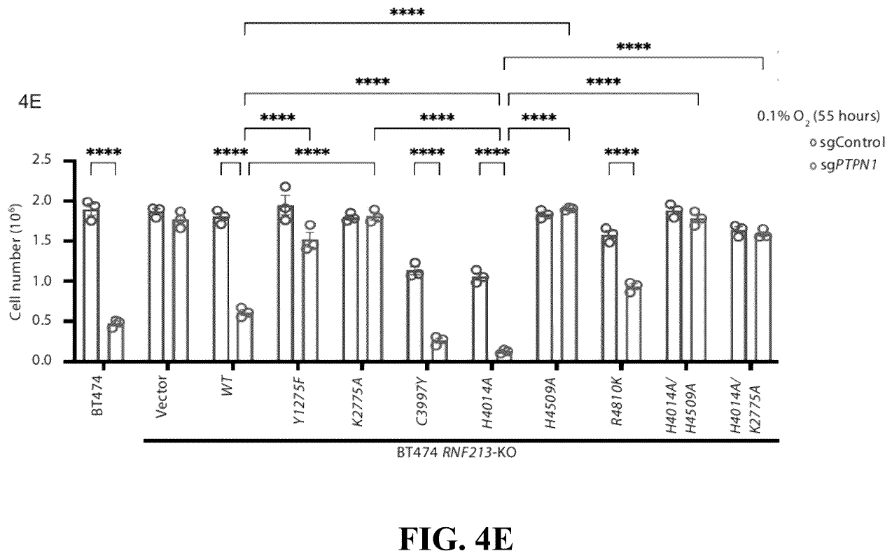

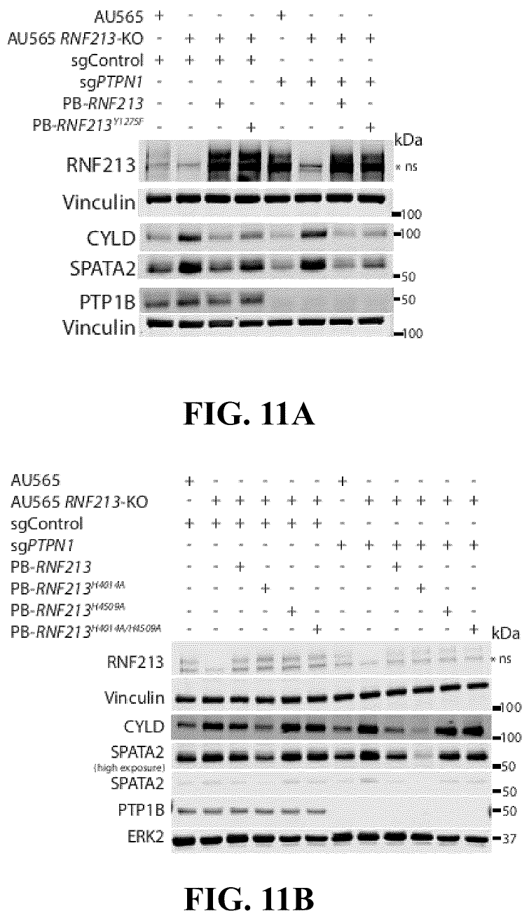

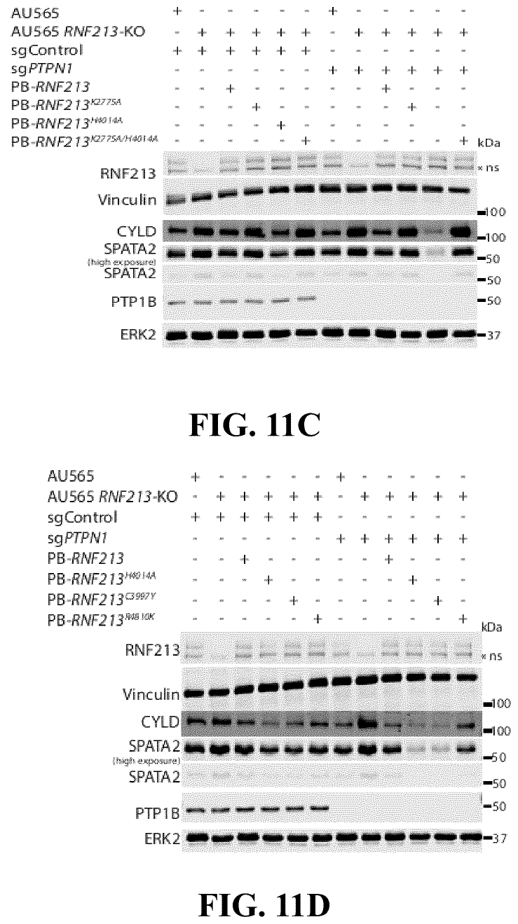

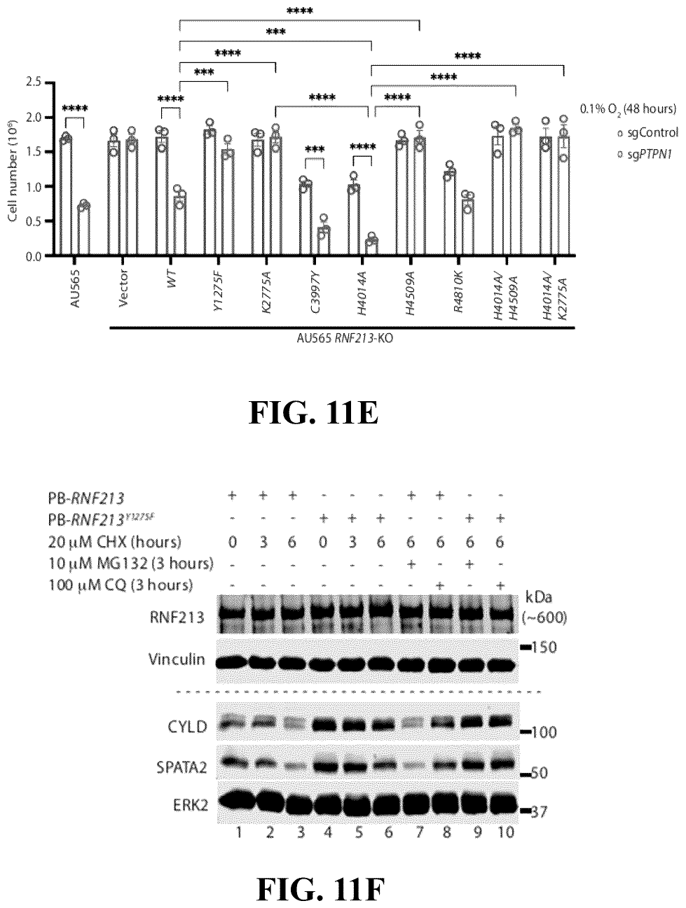

BT474 RNF213-KO (or AU565-RNF213-KO) cells were reconstituted with WT RNF213, RNF213^{Y1275F}, RNF213^{K2775A} (second AAA-ATPase domain mutant), RNF213^{H4014A} (RING-dead), RNF213^{H4509A} (RZ-dead), RNF213^{H4014A/H4509A} (RING/RZ-dead) or RNF213^{K2775A/H4014A} (ATPase/RZ-dead) to determine which RNF213 domains are required for hypoxia hypersensitivity. CRISPR/Cas9 was used to generate PTPN1-KO pools of each cell line. The resultant cell lines/pools were subjected to hypoxia, and lysates were analyzed by immunoblotting. WT RNF213, but not RNF213^{Y1275F} promoted CYLD and, to a lesser extent, SPATA2 degradation in BT474 (FIG. 4A) and AU565 cells (FIG. 11A). Consistent with post-translational regulation, CYLD/SPATA2 degradation was enhanced in cycloheximide (CHX)-treated cells that expressed WT RNF213 compared with those expressing RNF213Y1275 (FIG. 11F, lanes 3, 6). Chloroquine (CQ), but not MG132, blocked CYLD/SPATA2 degradation in the presence of CHX, indicating lysosomal degradation (FIG. 11F, lanes 3, 7, 8). Moreover, degradation was increased by PTP1B deficiency. RZ-dead or RING/RZ-dead RNF213 were unable to promote CYLD or SPATA2 degradation, whereas compared with WT RNF213, the RING-dead mutant enhanced degradation (FIG. 4B and FIG. 11B), especially in the PTPN1-KO background. Previous work showed that RNF213 ATPase activity is required for RZ domain-mediated ubiquitin ligase activity²⁹. Given the finding that RING-dead mutations show RZ domain gain-of function, it was hypothesized that an active ATPase domain should also be required for substrate degradation by RING-dead mutants. Indeed, a compound ATPase/RING domain mutant (RNF213^{K2775A/H4014A}) was unable to degrade CYLD or SPATA2 in the presence or absence of PTP1B (FIG. 4C and FIG. 11C). The effects of select MMD SNPs that affect the RING domain of RNF213^{23,48,49} was tested by restoring doxycycline-inducible expression of RNF213^{C3997Y} (highly penetrant) and RNF213^{R4810K} (most common, low penetrance) to RNF213-KO BT474 and AU565 cells. The highly penetrant MMD SNP had similar effects on SPATA2 and CYLD to RING-dead RNF213 (FIG. 4D and FIG. 11D).

The effect of each mutant on hypoxia sensitivity in the presence or absence of PTPN1-KO was tested. Consistent with the above biochemical results, cells reconstituted with tyrosine phosphorylation site-, ATPase-, RZ-, compound ATPase/RZ-, or compound RING/RZ-, mutants failed to show hypoxia hypersensitivity in response to PTP1B deficiency (FIG. 4E and FIG. 11E). By contrast, RING-dead RNF213 or highly penetrant MMD SNPs showed increased hypoxic cell death even in PTP1B-replete cells and almost no survival upon PTPN1 deletion. The low penetrant, common MMD SNP RNF213^{R4810K} also showed increased cell death in hypoxia in the PTP1B-replete state, although hypersensitivity was not further enhanced by PTPN1 deletion (FIG. 4E, and FIG. 11E). Without wishing to be bound by theory, the R4810K mutation interferes with transmission of the stimulatory effect of Y1275 phosphorylation/oligomerization to the RZ domain. The data described herein indicates that tyrosine phosphorylation of RNF213 promotes ATPase-mediated oligomerization and activation of the RZ domain ubiquitin ligase activity, which in turn leads to CYLD/SPATA2 ubiquitylation, degradation, and cell death. The RING domain inhibits the RZ domain, and MMD SNPs abrogate this regulation and promote increased CYLD/SPATA2 degradation and hypoxia hypersensitivity.

### Example 5. The LUBAC Complex Also is Required for CYLD/SPATA2 Degradation by RNF213

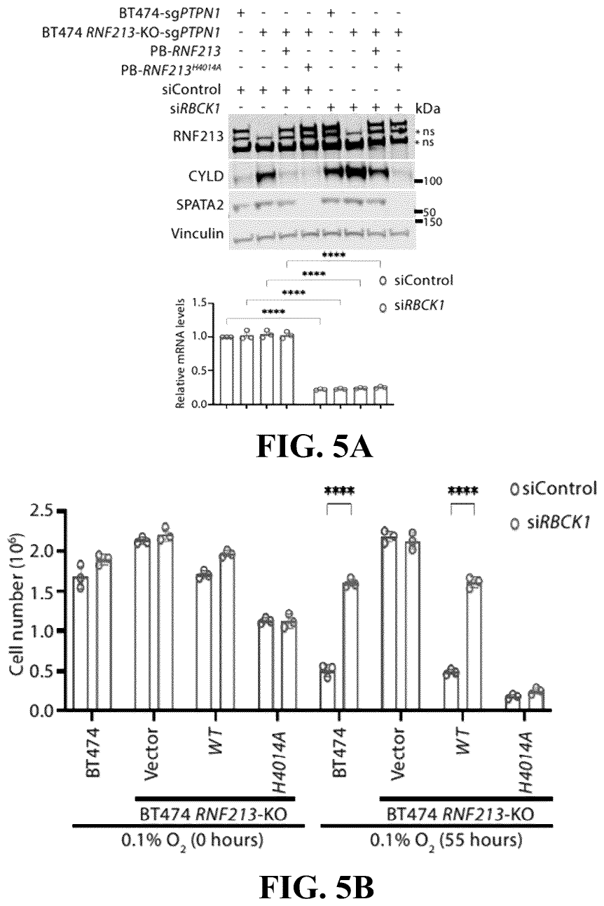

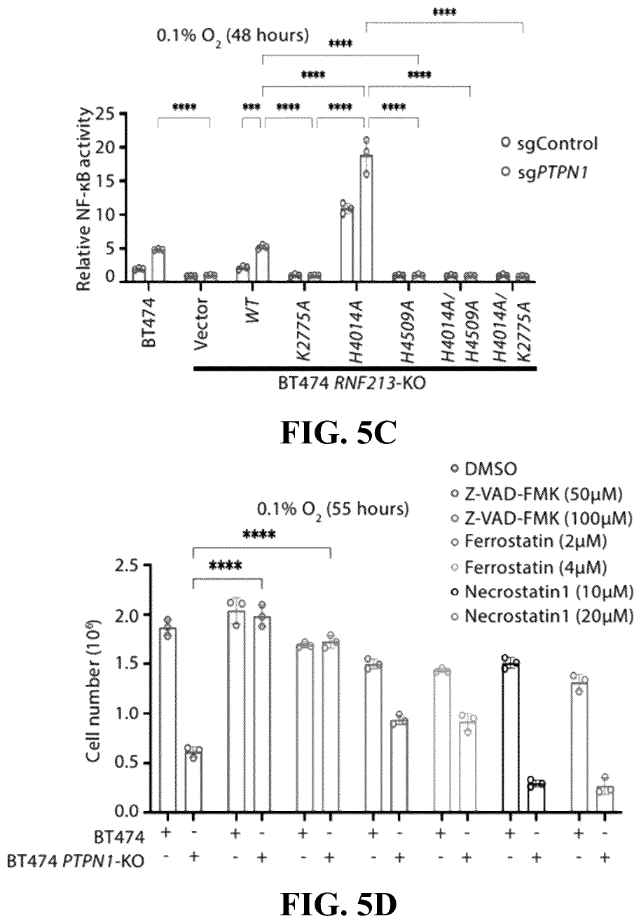

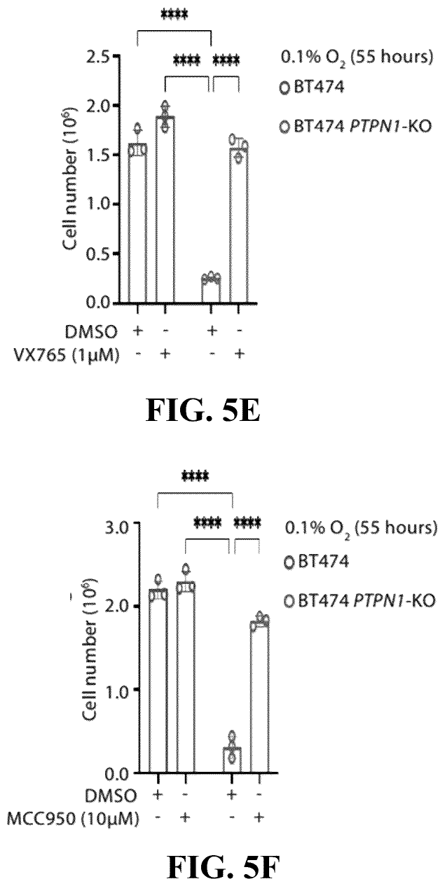

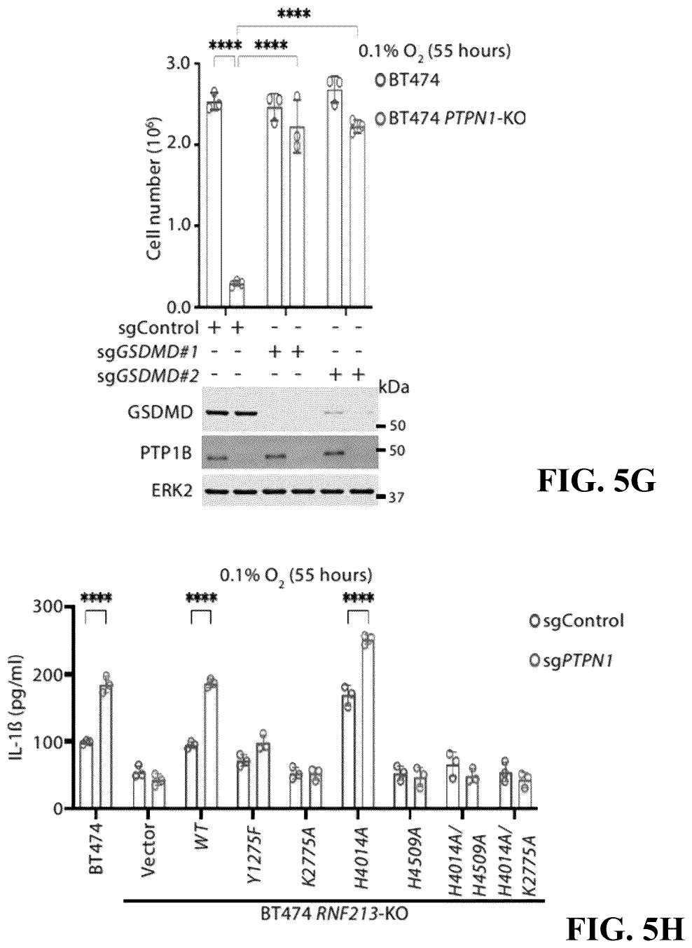

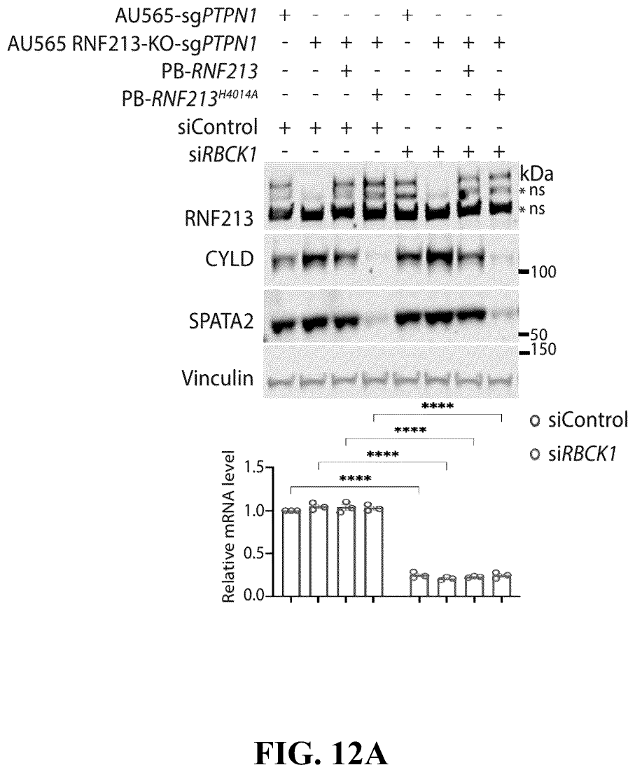

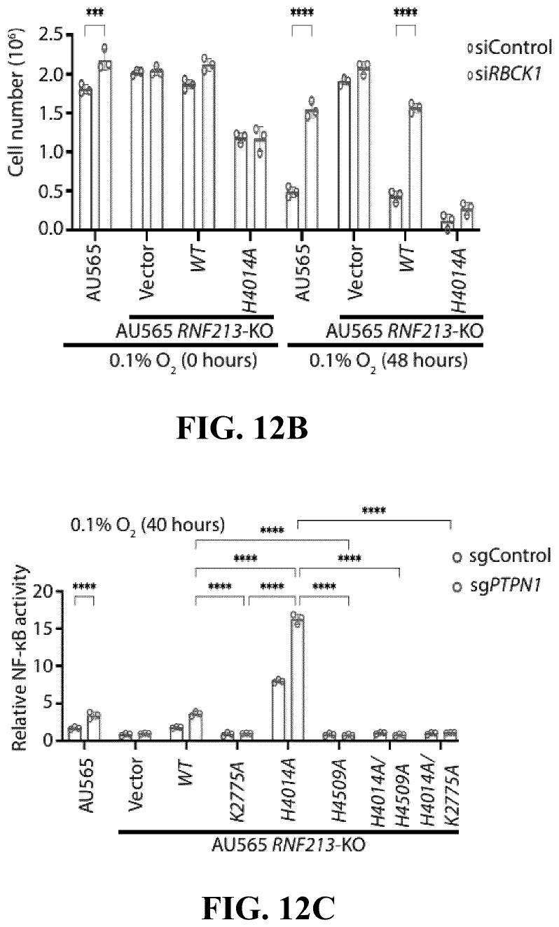

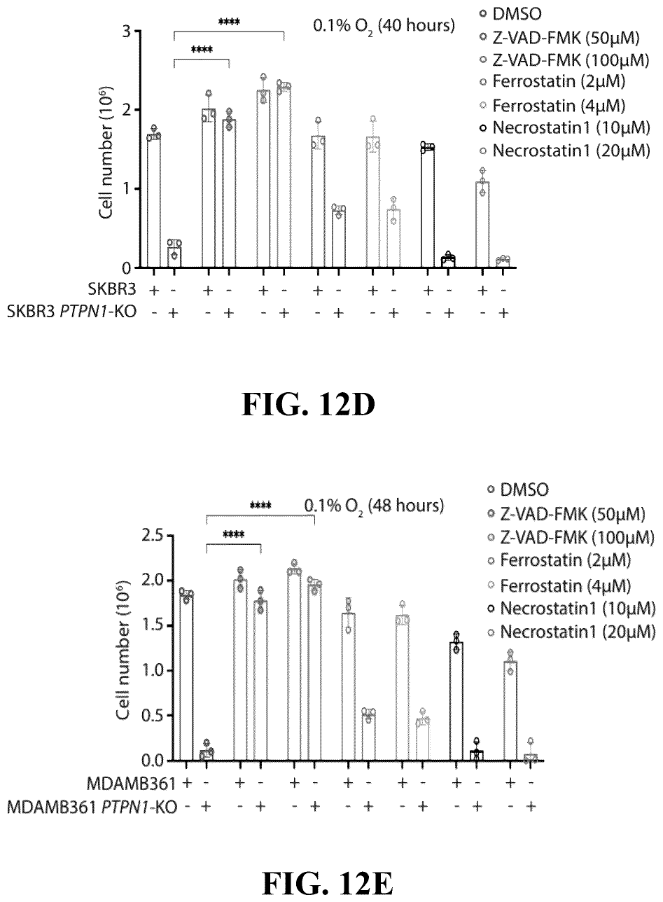

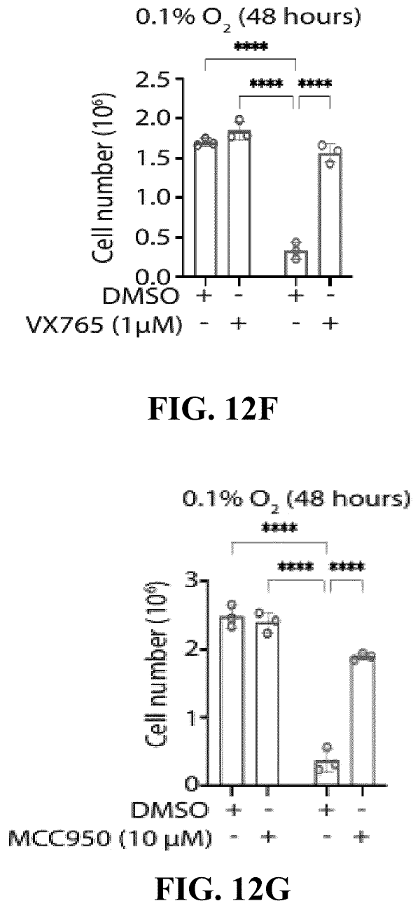

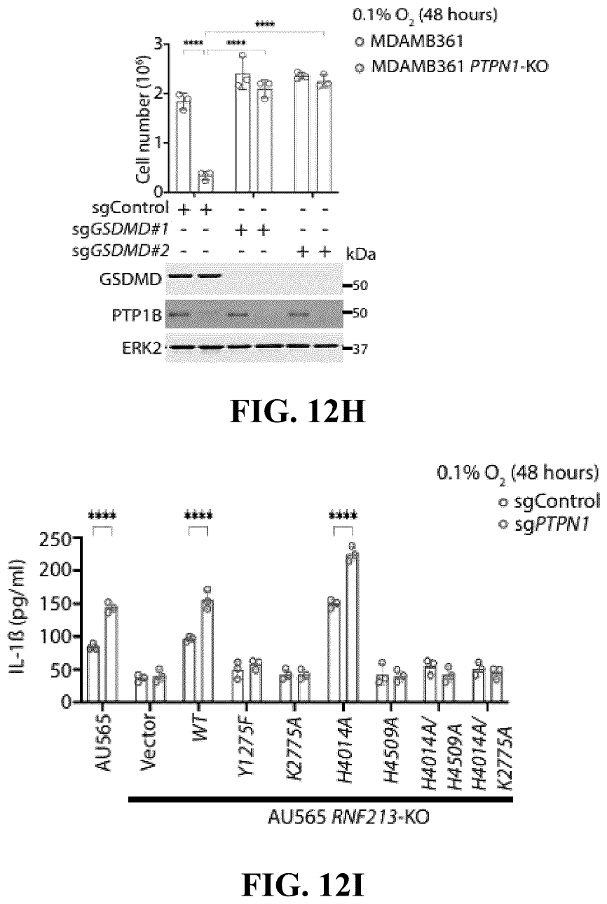

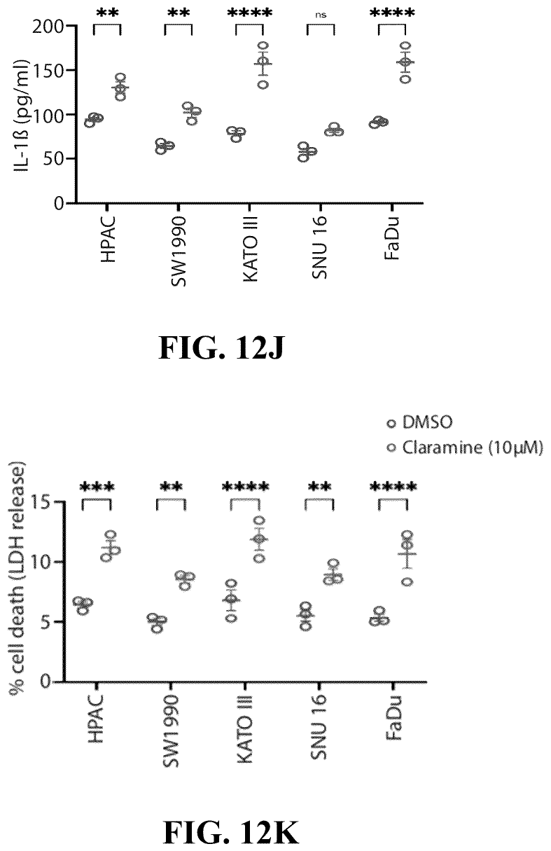

Recently, the RZ domain was found to ubiquitylate Salmonella lipopolysaccharide, most likely on its lipid component²⁹. The LUBAC complex then catalyzes further ubiquitylation, promoting bacterial clearance via autophagy. Knockdown of the LUBAC complex component RBCK1 prevented WT-RNF213-catalyzed degradation of CYLD and SPATA2 even in PTP1B-depleted BT474 or AU565 cells (FIG. 5A and FIG. 12A). The LUBAC complex was dispensable for CYLD/SPATA2 degradation by RING-dead RNF213. RBCK1 knockdown was unable to prevent hypoxia-hypersensitivity caused by reconstituting RNF213-KO cells with the RING-dead mutant (FIG. 5B and FIG. 12B).

### Example 6. RNF213, Via its RZ Domain, Promotes NF-κB Activation and Primes Cells for Pyroptosis

CYLD and SPATA2 are important negative regulators of NF-κB, so CYLD/SPATA2 degradation would be expected to increase NF-κB activity. A dual luciferase reporter assay was used to monitor NF-κB activity in RNF213-KO BT474 and AU565 cells reconstituted with WT RNF213 and various mutants with or without concomitant pooled PTPN1 deletion. RNF213-KO cells (Vector) failed to increase reporter activity when PTP1B was depleted, while reconstituting these cells with WT-RNF213 increased reporter activity in PTPN1-KO cells to levels similar to that of parental BT474 cells with PTPN1 deletion. RING-dead RNF213-reconstituted cells had markedly increased NF-κB activity even when PTPN1 was intact, and activity increased further upon PTPN1 deletion. Consistent with their effects on CYLD/SPATA2, RZ mutant, RING/RZ mutant, or ATPase/RZ mutant forms of RNF213 failed to induce NF-κB under any condition and might even have a dominant negative effect (FIG. 5C and FIG. 12C).

CYLD promotes apoptosis and necroptosis^{50,51}, and NF-κB typically promotes cancer cell survival⁵². Yet PTP1B depletion or inhibition increases cell death in hypoxia, while promoting CYLD degradation and enhancing NF-κB activity. The mechanism that PTP1B-deficient, hypoxic cells die, was evaluated by testing the effects of known inhibitors of various cell death pathways. Neither ferroptosis nor necroptosis inhibitors blocked hypoxia hypersensitivity in PTPN1-KO cells; however, the general caspase inhibitor Z-VAD-FMK prevented hypoxia-induced cell death (FIG. 5D). Similar results were obtained using SKBR3 or MDA-MB-361 cells (FIGS. 12D and 12E).

No increase in apoptosis (a caspase-dependent process) was previously observed in PTP1B-deficient breast cancer cells; instead, death appeared to be necrotic²². However, Z-VAD-FMK also inhibits “inflammatory caspases” (Caspases 1,4,5 in humans, Caspases 1 and 11 in mouse). Remarkably, treatment with VX765 (1 μM), a specific inhibitor of Caspase 1 and Caspase 4, completely blocked hypoxic cell death in PTPN1-KO BT474 cells (FIG. 5E). Similar results were observed using MDA-MB-361 cells (FIG. 12F).

NF-κB induces the expression of the inflammasome component NLRP3, as well as IL1B. The inflammasome, in turn, facilitates pro-IL-1β cleavage and release of the active cytokine, as well as the cleavage of Gasdermins⁵³. Cleaved Gasdermins oligomerize and form pores in cell membranes, leading to pyroptotic cell death. Notably, hypoxia hypersensitivity of PTPN1-KO BT474 or MDA-MB-361 cells was also blocked by treatment with the NLRP3 inhibitor MCC950 (FIG. 5F and FIG. 12G) or by knockout of GSDMD with either of two sgRNAs (FIG. 5G and FIG. 12H). PTPN1-KO also increased IL-1b production in RNF213-WT-reconsituted BT474 or AU565 cells (FIG. 5H and FIG. 121). IL-10 levels increased even further in cells expressing RING-dead RNF213, but there was no detectable increase in cells expressing RNF213 that cannot be tyrosine phosphorylated (Y1275F) or in RZ-dead, ATPase-dead, RING-dead and RZ-dead or RING-dead and ATPase-dead mutants. Increased release of IL-1β and LDH from other cancer cells treated with PTP1B inhibitor was also detected (FIG. 20, FIGS. 12J and 12K).

### Example 7. Hypoxia-Driven ER Stress Sends the Second Signal for Pyroptotic Cell Death

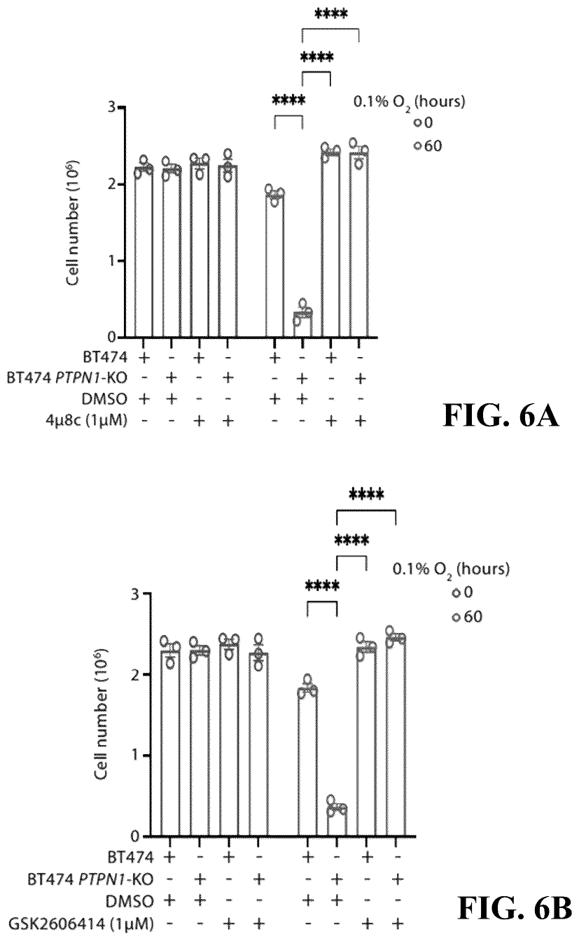

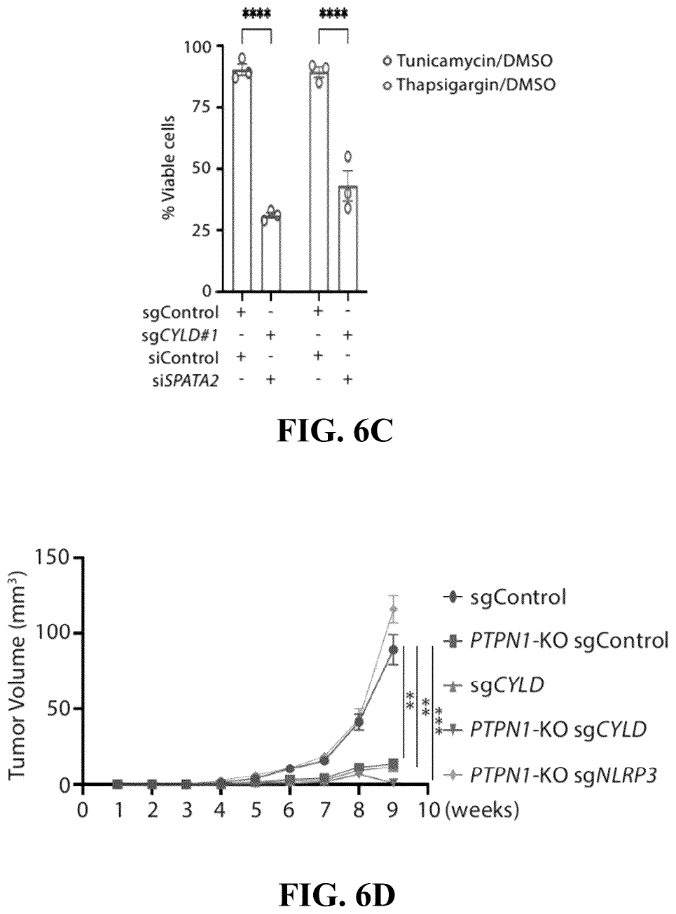

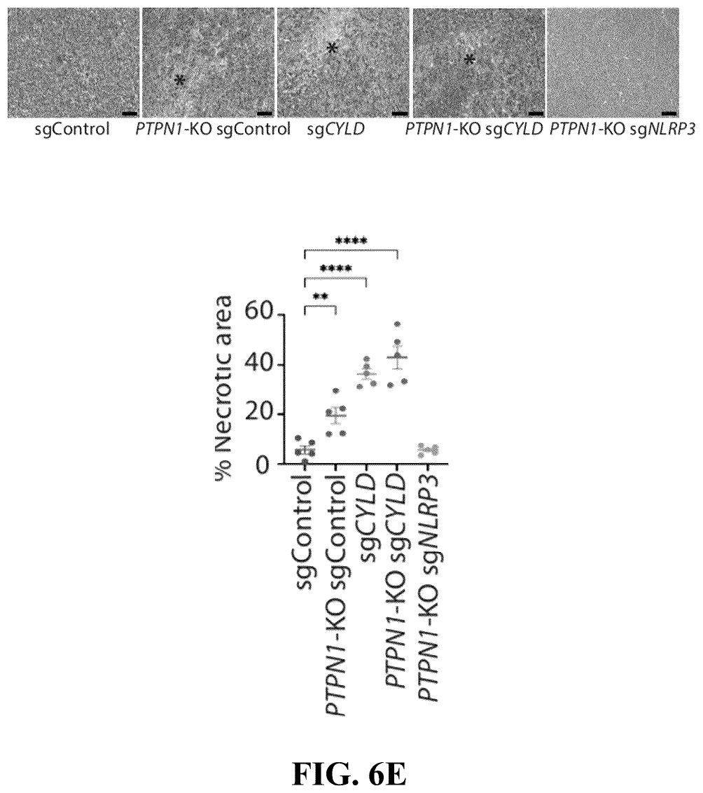

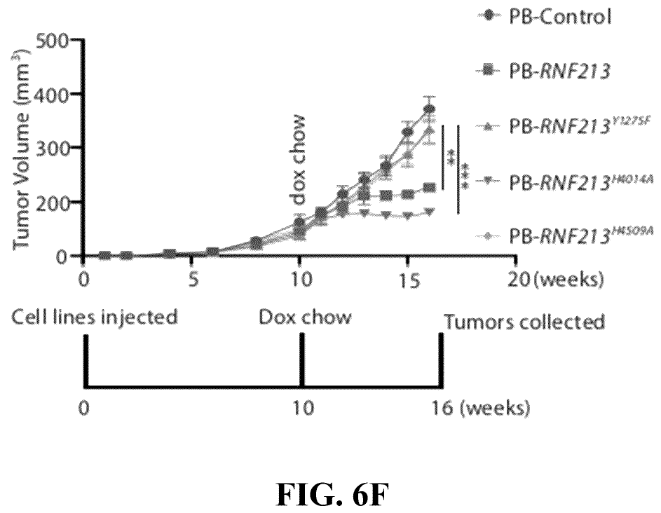

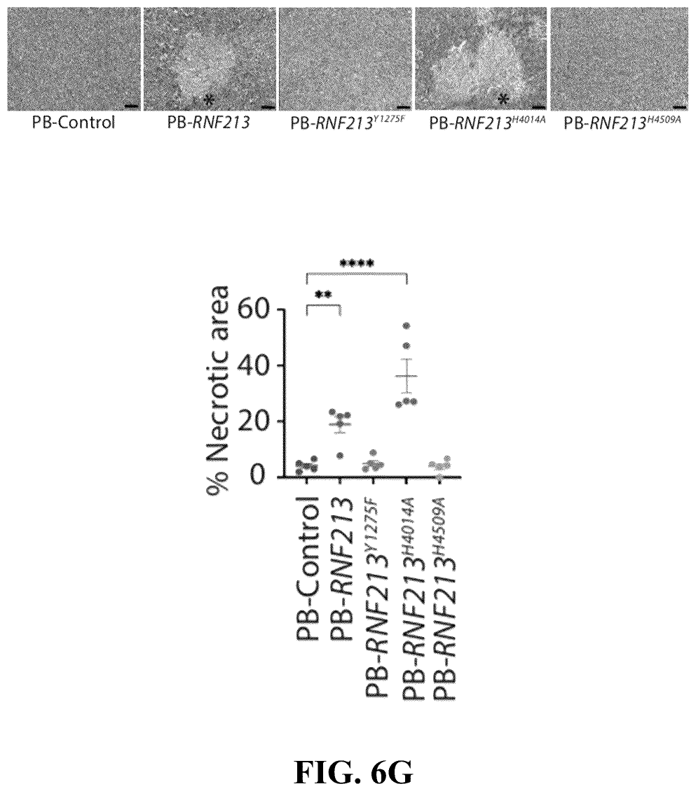

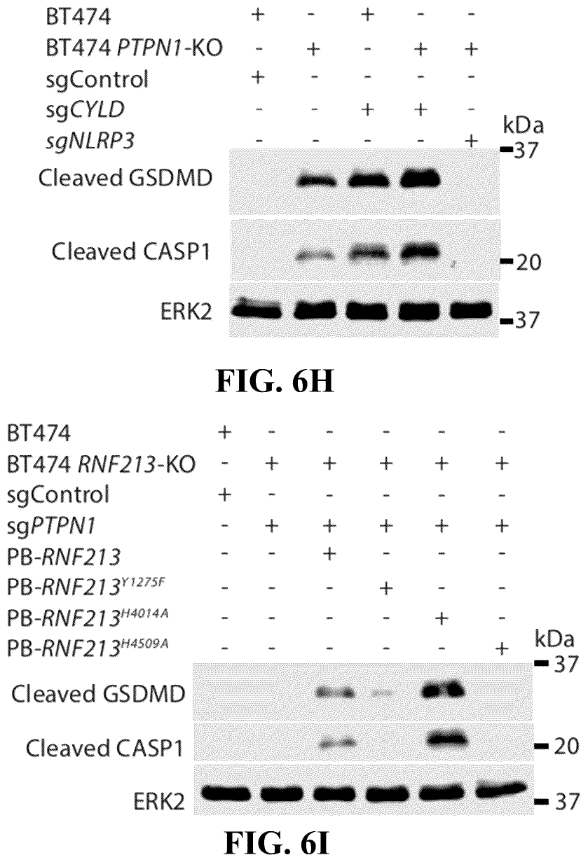

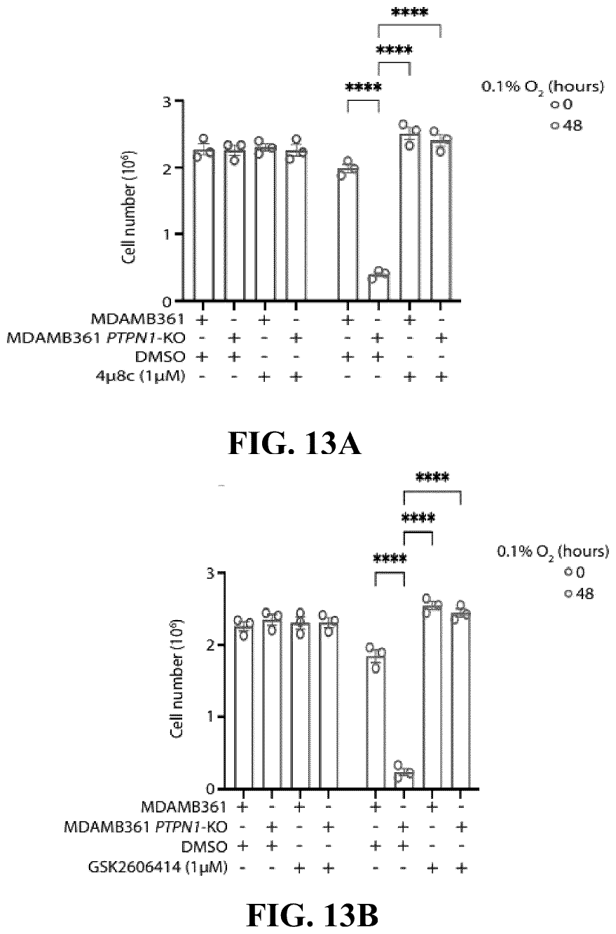

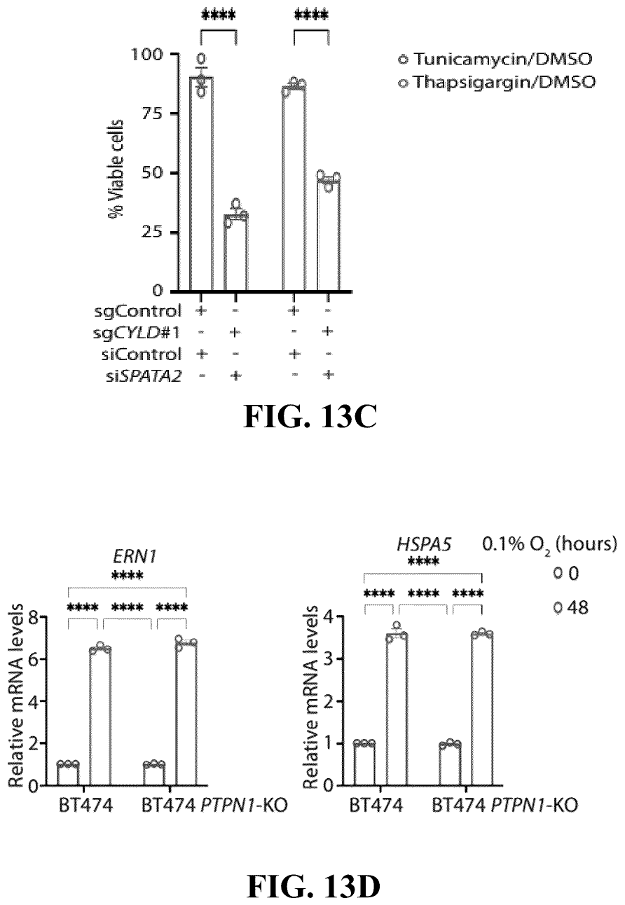

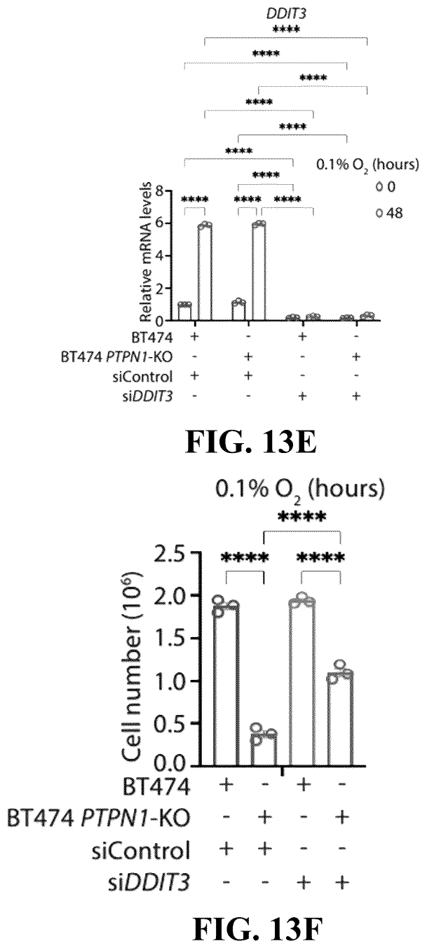

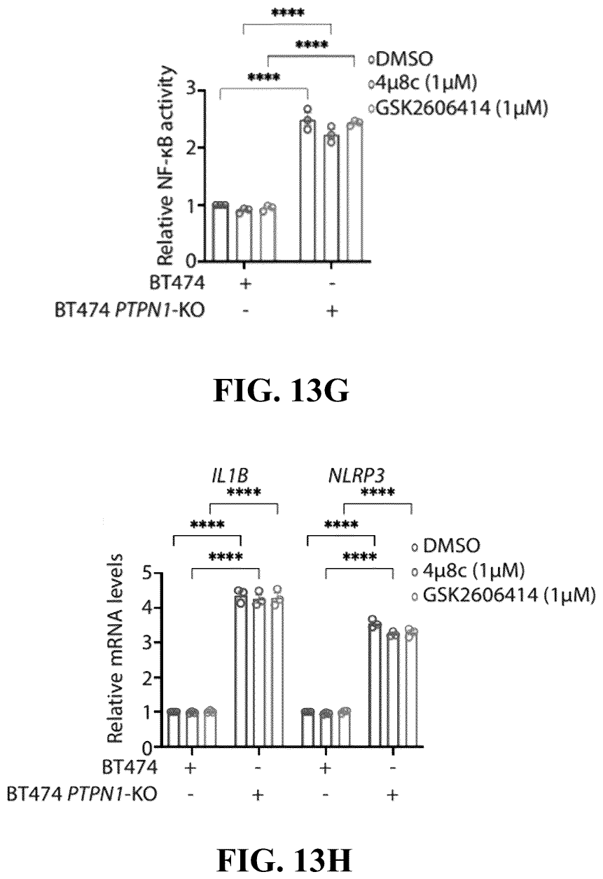

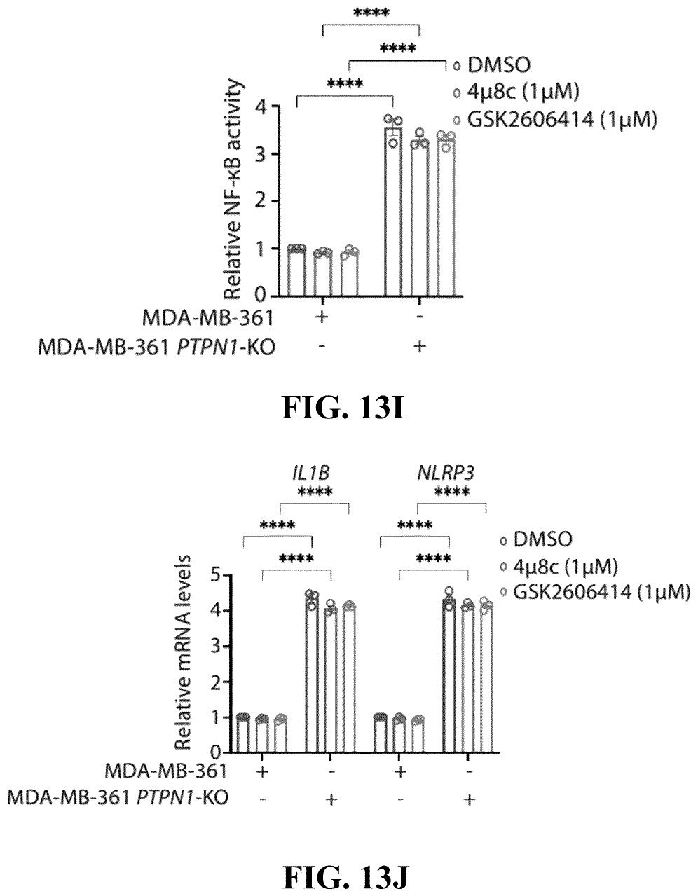

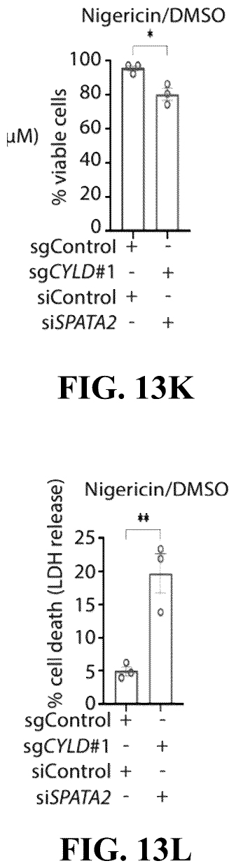

NF-κB can prime cells for pyroptosis, but a “second signal” is required to trigger inflammasome assembly/inflammatory caspase activation, Gasdermin cleavage, and cell death. Several stimuli can provide the second signal, including ER stress, which is evoked by O₂ levels ≤1%⁵⁴. Remarkably, PTPN1-KO cells treated with the IRE1α inhibitor 4m8c were resistant to hypoxic cell death compared with DMSO control-treated cells (FIG. 6A). Similar results were obtained using MDA-MB-361 cells (FIG. 13A) or by treating PTPN1-KO BT474 or MDA-MB-361 cells with the PERK inhibitor GSK2606414 (FIG. 6B and FIG. 13B). Parental BT474 cells and BT474 cells rendered SPATA2- and CYLD-deficient were treated to test whether ER stress is sufficient to send the second signal. The cells were treated to mimic the effects of PTP1B deficiency in hypoxia (by CYLD-KO and SPATA2 siRNA) with tunicamycin or thapsigargin in normoxia. Both agents induced cell death selectively in SPATA2- and CYLD-deficient cells (FIG. 6C). Analogous results were obtained using cognate MDA-MB-361 cells (FIG. 13C). Moreover, the ER stress markers ERN1 (encoding IRE1α, HSPA5 (BIP)), and DDIT3 (CHOP) were induced in hypoxic HER2+ breast cancer cells regardless of their PTP1B status (FIGS. 13D and 13E). CHOP is also implicated as an effector of ER stress-induced cell death and inflammation; notably, DDIT3 knockdown partially (but incompletely) reversed hypoxia hypersensitivity in PTPN1-KO cells (FIG. 13F). By contrast, ER stress did not affect NF-κB reporter activity or target gene expression in PTPN1-KO BT474 or MDA-MB-361 cells (FIGS. 13G-13J). Another known pyroptosis activator, nigericin, also caused cell death in normoxic parental and SPATA2/CYLD-deficient BT474 cells (FIGS. 13K and 13L).

### Example 8. RNF213 Triggers Pyroptosis in HER2+ Breast Tumors

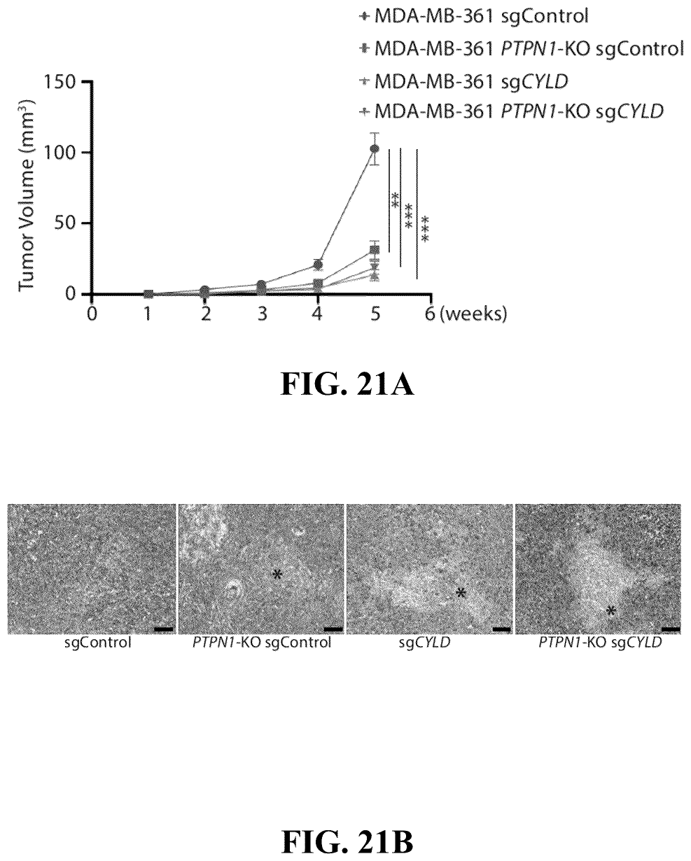

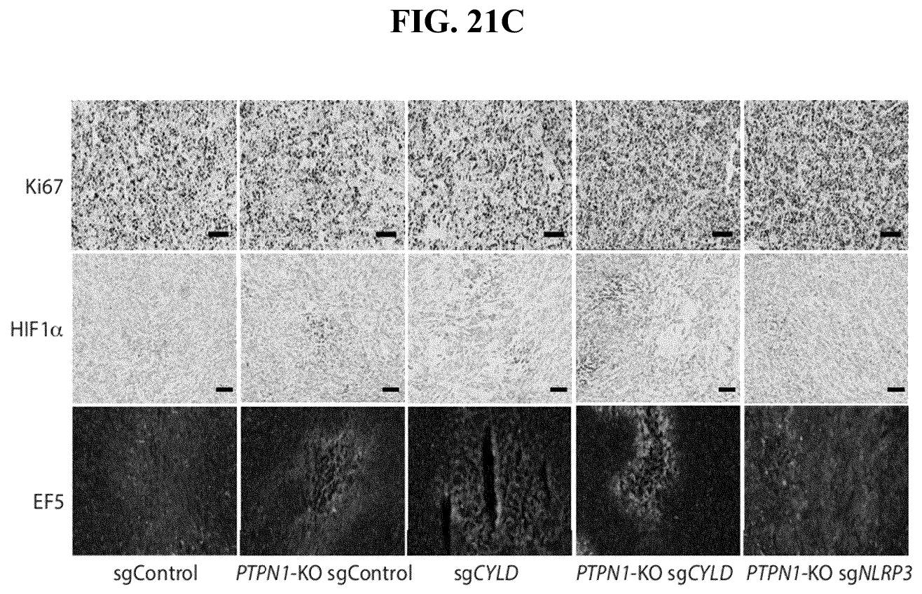

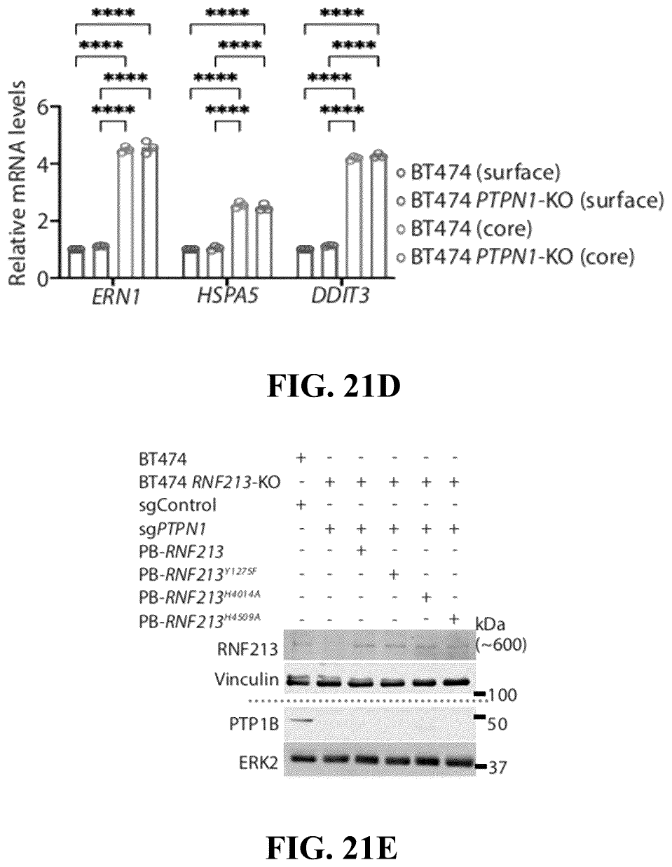

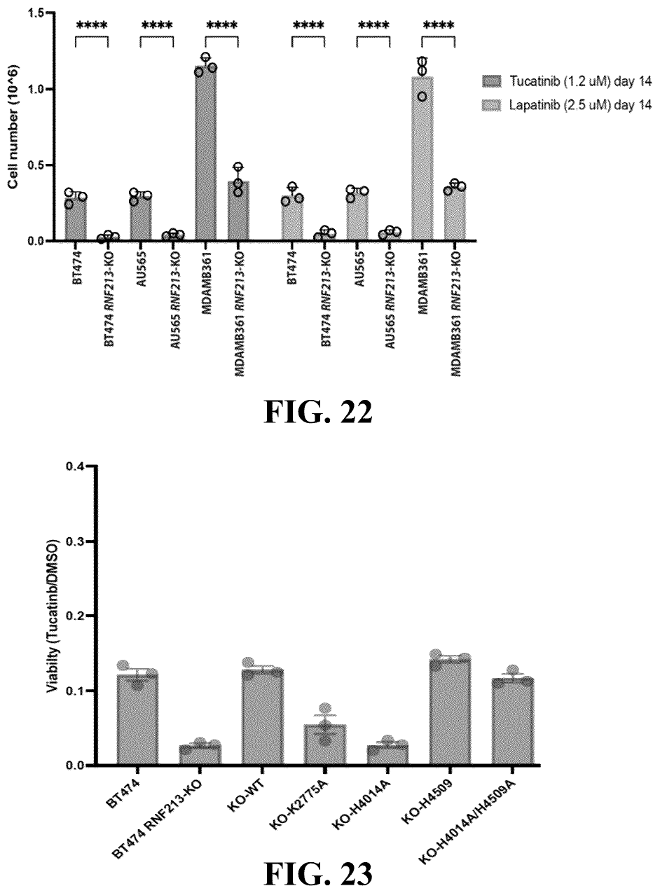

Finally, to explore the pathophysiological relevance of the above-described observations, parental, PTPN1-KO, CYLD-KO, and PTPN1-KO-CYLD-KO BT474 and MDA-MB-361 cells were implanted into athymic nu nu mice and tumor development was monitored. Absence of PTP1B, CYLD, or both, markedly inhibited tumor growth (FIG. 6D and FIG. 21A). Importantly, NLRP3 deletion was epistatic to PTPN1 deficiency, establishing the essentiality of the inflammasome for inhibiting tumor growth (FIG. 6D, PTPN-KO sgNLRP3). PTPN1-KO, CYLD-KO or PTPN1/CYLD-KO BT474- and MDA-MB-361-derived tumors showed increased necrosis (by H&E staining) compared with those from parental or PTPN1-KO/NLRP3-KO cells, even though the former were much smaller (FIG. 6E, left panels, quantified at right and FIG. 21B). Consistent with these findings (and in vitro experiments described herein), tumor cores were hypoxic (as indicated by HIF1α and EF5 staining, FIG. 21C), and ER stress genes were induced (FIG. 21D). By contrast, Ki67 and EdU staining were unaffected by tumor cell genotype (FIGS. 21C and 21E).

To determine which RNF213 domains are required for tumor death, RNF213-KO sgPTPN1 BT474 cells reconstituted with doxycycline-inducible WT RNF213, RNF213^{Y275F}, RNF213^{H4014A}, or RNF213^{H4509A} were implanted. Tumors were allowed to establish, but when volumes reached ˜100 mm³, mice were switched to doxycycline (Dox)-containing chow to induce WT or mutant RNF213 expression. Expression of WT or RING-dead RNF213 impaired tumor growth; by contrast, tumors expressing phosphosite- or RZ-dead mutant-reconstituted cells grew like RNF213-KO tumors (FIG. 6F and FIG. 21E). WT RNF213- and RNF213^{H4014A}-reconstituted tumors showed marked necrosis (FIG. 6G, left panels, quantified at right). Immunoblots of xenograft lysates confirmed that PTPN1- or CYLD-KO induced cleaved CASP1 and cleaved GSDMD (FIGS. 6H and 61), indicating pyroptotic cell death.

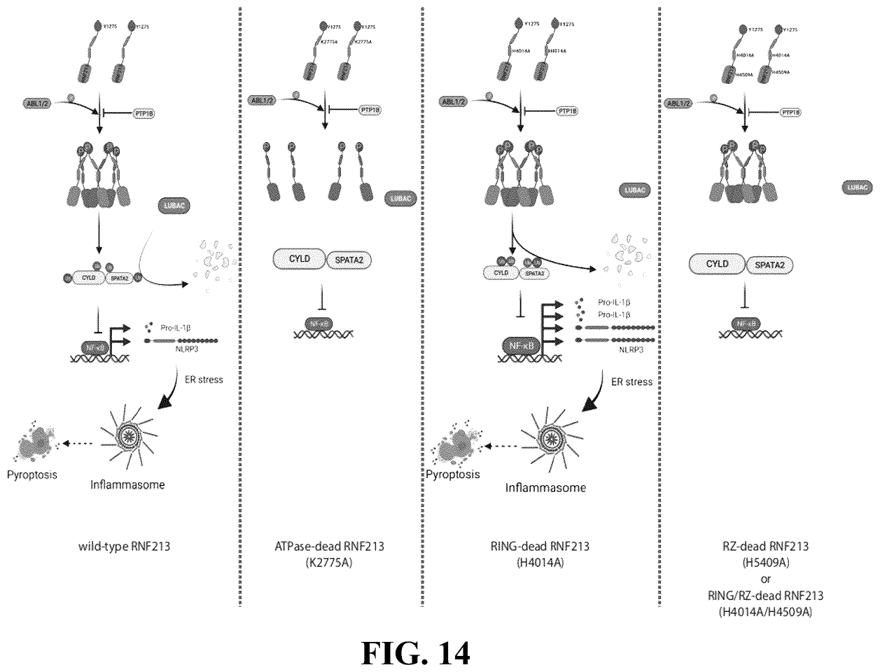

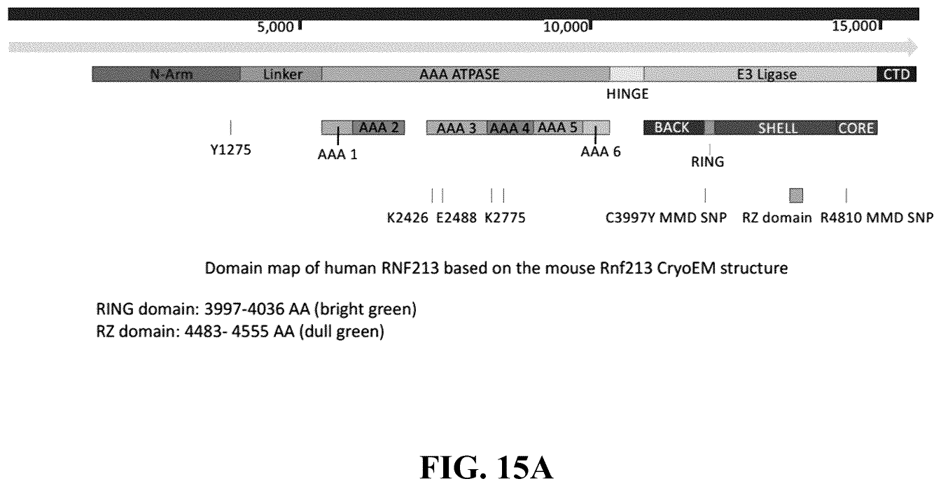

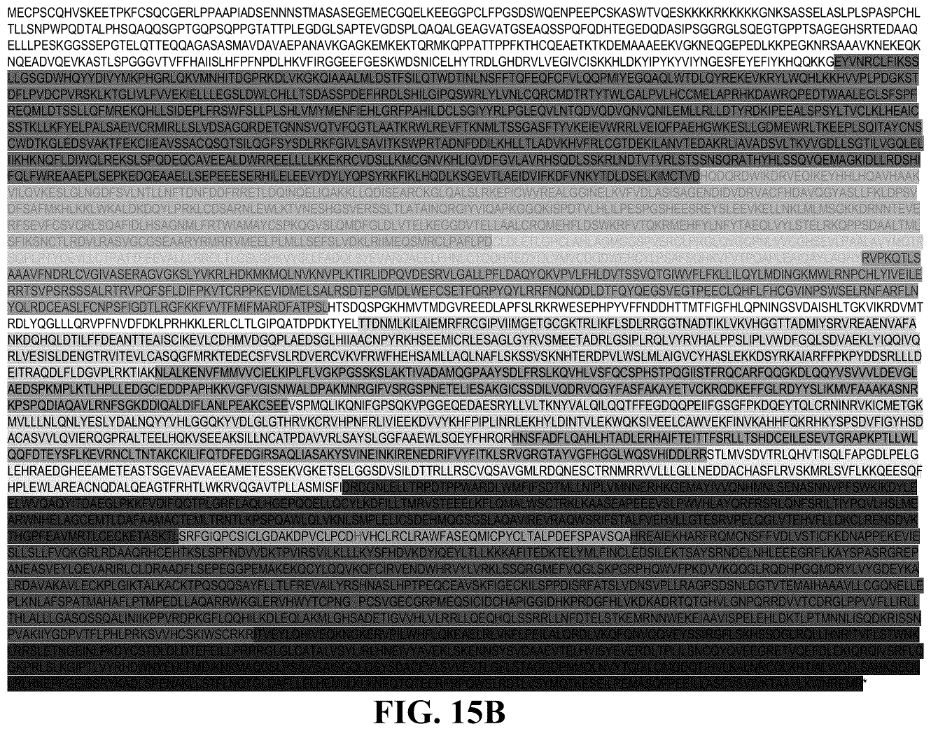

Described herein includes unique mechanisms of inflammatory cell death in breast cancer cells exposed to hypoxia (FIG. 14). RNF213, the product of the major susceptibility gene for MMD, was found to be phosphorylated at Y1275. Y1275 phosphorylation promotes RNF213 oligomerization, and is reciprocally regulated by ABL1,2 and PTP1B. Oligomerization activates the RNF213 RZ domain to catalyze CYLD/SPATA2 ubiquitylation and degradation. The LUBAC complex also is required for RNF213-initiated degradation of CYLD/SPATA2, but this requirement is abrogated in MMD SNPs or by engineered mutants that inactivate the RNF213 RING. Decreased CYLD/SPATA2 levels lead to increased NF-κB activation in hypoxia, which promotes NLRP3 transcription. A second signal, most likely hypoxia-induced ER stress, promotes inflammasome assembly and activation, leading to pro-IL-1β processing/secretion and GSDMD-dependent pyroptotic cell death. The results described herein provide new insights into the hypoxia response, RNF213 regulation, and MMD pathogenesis. The pathway revealed here, possibly in concert with other RNF213 interactors identified by proximity ligation screen, could also contribute to more common disorders, such as myocardial infarction/stroke and lipotoxicity (e.g., hepatic steatosis).

Hypoxic regions in tumors typically are therapy-resistant and provide a cellular reservoir for tumor recurrence^{3,7}. PTP1B inhibition or degradation (e.g., using PROTAC technology⁵⁵) can kill these hypoxic cells and could be useful in combination with other anti-neoplastics. NF-κB signaling is activated in hypoxia through IKKβ, but the detailed mechanism has remained unclear. Scholz et al.⁵⁶ reported that components of the TRAF6 complex (OTUB, UVEV1a) associate with PHD1 and that multiple NF-κB pathway components are ubiquitylated. The results described herein indicate that hypoxia also promotes NF-κB activation via degradation of a major deubiquitylase complex. HPV-infected cells activate NF-κB in hypoxia via CYLD degradation⁵⁷. Degradation requires E6, but not E6AP, the ubiquitin ligase that typically mediates E6 action.

Importantly, PTP1B inhibition/deficiency induces pyroptosis, an inflammatory form of cell death, which could help “turn cold tumors hot” and promote the anti-tumor immune response⁵³. Inflammation also can suppress tumor immunity⁵³; notably, PTP1B inhibition/deficiency increases IL-1β production, which promotes immunosuppression by myeloid cells⁵⁸. PTP1B also has direct effects in myeloid cells⁵⁹ and lymphocytes^{60,61}; whether the PTP1B/RNF213/CYLD/SPATA2 pathway is active in these cells remains unclear. A PTP1B/TCPTP inhibitor is in clinical trials as an anti-neoplastic agent³⁴ (NCT04777994), mainly because of its predicted immunostimulatory effects^{60,62}. Elucidating the cell-autonomous and non-autonomous effects of such agents, including if/how TCPTP inhibition affects RNF213, should aid in their rational deployment. It also will be important to test whether PTP1B controls hypoxia sensitivity in other tumor types, particularly pancreas cancer, which is notoriously hypoxic⁶³.

Most previous studies of RNF213 have focused on its E3-ubiquitin ligase domains, although it is known that its E3 ligase activity requires ATPase function and, most likely, hexamerization²⁹. Oligomerized RNF213 serves as a sensor for ISG15⁶⁴; however, reciprocal control of Y1275 phosphorylation by ABL1/2 and PTP1B is the first example of a signaling pathway regulating RNF213 oligomerization/E3 activation. The RZ domain, previously implicated in lipid ubiquitylation, was found to have protein targets (CYLD/SPATA2).

The results described herein also yield several new clues into MMD pathogenesis. RNF213-catalyzed CYLD/SPATA2 degradation provides a mechanistic explanation for earlier reports that RNF213, and to a greater extent, MMD-associated SNPs, activate NF-κB³⁰. MMD is characterized by precocious carotid occlusions and stroke. The most common RNF213 SNPs activate the RZ domain and confer LUBAC-independence on CYLD/SPATA2 degradation (and consequently, NF-κB activation and increased inflammation) could explain how these alleles promote vascular disease. MMD is also associated with Graves' disease, systemic lupus erythematosus, Sjögren's syndrome, multiple sclerosis, rheumatoid arthritis, and other immune/inflammatory disorders^{65,66}. Bypassing the LUBAC complex requirement for CYLD/SPATA2 degradation/NF-κB signaling could help explain these associations.

Finally, disordered regulation of SPATA2/CYLD and possibly other RNF213 interactors could be relevant to more common diseases and might be targeted therapeutically. For example, RNF213 deficiency abrogates lipotoxicity caused by saturated fatty acids in vitro³². Saturated fatty acids also can provide the “second signal” for pyroptosis⁶⁷ and induce ER stress⁶⁸. In some embodiments, RNF213 degraders, ATPase inhibitors, or RZ inhibitors might have therapeutic benefit in, for example, non-alcoholic fatty acid disease. Several other RNF213 interactors (MAP4K4, SPTLC1, SARS2) are associated with atherosclerotic disorders including stroke.

### Example 9. The Number of Drug-Tolerant Persister Cells (DTPs) Following HER2 Inhibitor Treatment is Reduced in RNF213 Knockout (KO) BT474 Cells as Compared to Wild Type RNF213 BT474 Cells

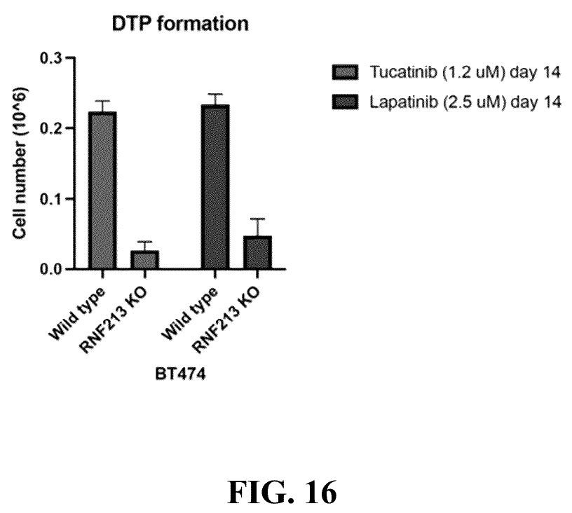

As shown in FIG. 16, RNF213 KO cells show a significant reduction in cell number compared to wild type RNF213. Samples including 4×10⁶ BT474 and BT474 RNF213-KO cells were seeded on a 10 cm plate. 24 hours after seeding, cells were treated with HER2 inhibitors tucatinib and lapatinib for 14 days at either 1.2 μM or 2.5 μM. After 14 days, remaining viable cells were counted. These results indicate that RNF213-KO cells are less likely to form drug-tolerant persister cells (DTPs) post HER2 inhibitor treatment which is the prescribed drug for treating HER2+ BC patients.

### Example 10. TNK1 Phosphorylates RNF213 and Positively Regulates RNF213 Activity

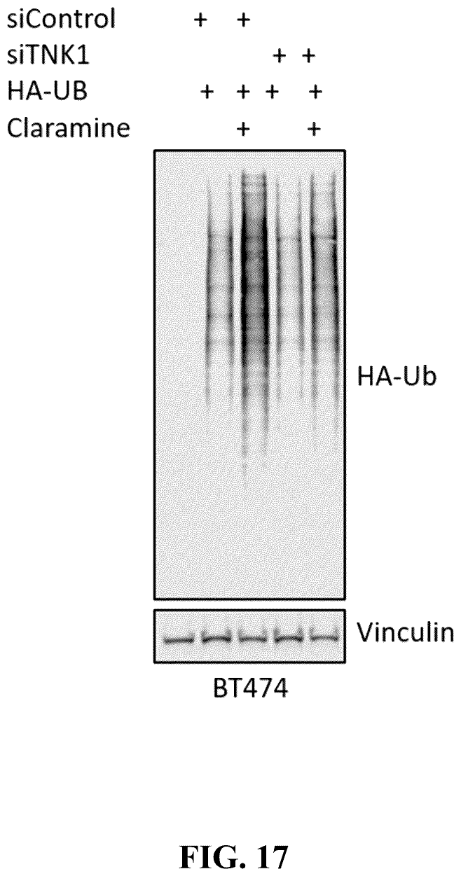

Previously, it has been reported that RNF213 increases global ubiquitylation in absence or inhibition of PTP1B. Since TNK1 is a major tyrosine kinase that could affect global ubiquitylation as it directly binds to ubiquitin, whether TNK1 regulates global ubiquitination in PTP1B inhibited cells was tested. The results showed that TNK1-KD suppressed the increased global ubiquitylation in PTP1B inhibited cells (FIG. 17).

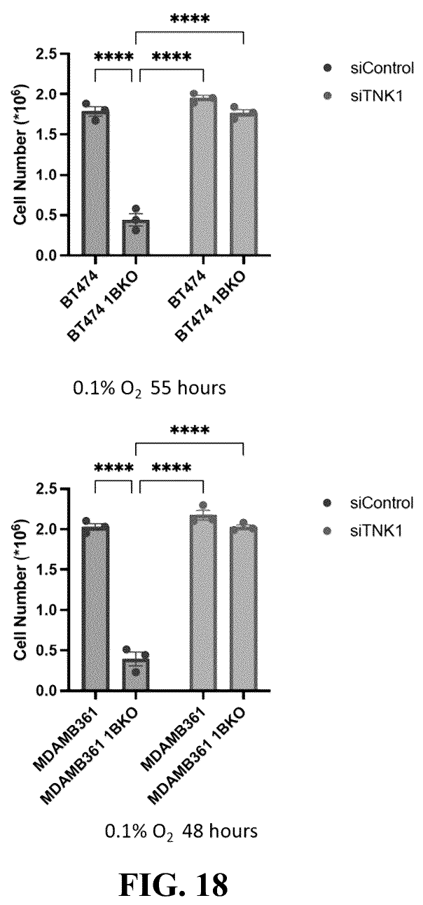

Since RNF213 promotes hypoxia hypersensitivity in PTPN1-KO cells, the effects of TNK1-KD in PTPN1-KO cells on hypoxia hypersensitivity was tested. The results suggested that TNK1-KD impaired the hypoxia hypersensitivity of BT474 PTPN1-KO cells (top) and MDA-MB-361 PTP1B-KO cells (bottom) (FIG. 18).

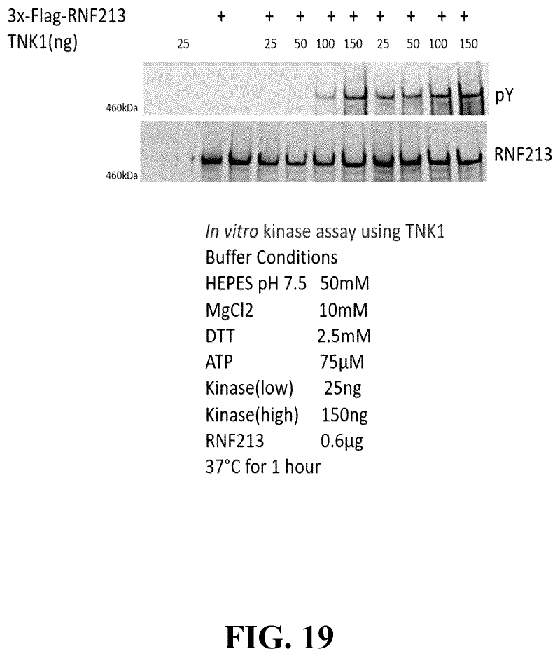

Further, whether the effects of TNK1 on hypoxia viability were regulated through RNF213 phosphorylation was tested. For this, purified RNF213 was subjected to an in vitro kinase assay. The results suggested that TNK1 phosphorylates RNF213 in a dose dependent manner (FIG. 19).

Overall, results described in the present Example suggest that TNK1 phosphorylates RNF213 and positively regulates RNF213 activity.

Below are the methods used in the Examples described above.

### Cell Culture and Cell Line Generation

Sources of the breast cancer cell lines used here were described⁶⁹; all are maintained as frozen stocks by the Neel Laboratory. Except where indicated, cells were cultured in DMEM+10% FBS with 100 U/ml penicillin and 100 mg/ml streptomycin at 37° C. in 5% CO₂ and tested monthly for mycoplasma by PCR.

TABLE 1
Cell lines

| | | |
|---|---|---|
| HeLa Flp-In-TRex | DMEM | +10% FBS |
| HEK293T | DMEM | +10% FBS |
| BT474 | DMEM | +10% FBS |
| SKBR3 | DMEM | +10% FBS |
| MDA-MB-361 | DMEM | +10% FBS |
| AU565 | DMEM | +10% FBS |
| MCF7 | DMEM | +10% FBS |
| CAMA1 | DMEM | +10% FBS |
| MDA-MB-175 | DMEM | +10% FBS |
| MDA-MB-134 | DMEM | +10% FBS |
| BT20 | DMEM | +10% FBS |
| MDA-MB-453 | DMEM | +10% FBS |
| MDA-MB-157 | DMEM | +10% FBS |
| MDA-MB-468 | DMEM | +10% FBS |
| HPAC | RPMI | +10% FBS |
| SW1990 | DMEM | +10% FBS |
| KATO III | RPMI | +20% FBS, |
| | | 2 mM |
| | | Glutamine |
| SNU 16 | RPMI | +10% FBS |
| FaDu | DMEM | +10% FBS |

Isogenic HeLa Flp-In T-Rex RNF213-KO cells that inducibly express wild-type RNF213 or RNF213 variants in response to doxycycline (DOX) were generated by co-transfecting pcDNA5/FRT/TO-based expression constructs with 3×-Flag tagged RNF213 or RNF213 variant with pOG44, which directs constitutive expression of Flp recombinase (FLP). were selected by adding hygromycin B (200 μg/ml) 48 hours post-transfection. Single cell clones of RNF213-deleted HeLa, BT474, and AU565 cells, or of PTPN1-deleted BT474, SKBR3, and MDAMB361 cells, were generated by using CRISPR/Cas9 technology⁷⁰. Briefly, sgRNAs targeting each gene were cloned into the BbsI site of pSpCas9 (BB)-2A-Puro (PX459; Addgene), and cells were transfected with each clone using Lipofectamine™ 3000 Transfection Reagent (ThermoFisher Scientific). After 48 hours, cells were diluted into 96-well plates (1 cell/well) containing 48-hour conditioned media from the same line in addition to standard media (1:1). Knockout clones were confirmed by immunoblotting. The sgRNA sequences are shown in Table 2.

TABLE 2
siRNA and guide RNA (gRNA)

| | | SEQ | |
|---|---|---|---|
| siRNA and gRNA | | ID | |
| used in study | Target sequence | NO: | Catalog |
| sgRNF213 | ACAATGGCGTCGGCCTCGGA | 4591 | Synthesized from |
| | | | Millipore-Sigma |
| sgPTPN1 | ACCTTCGATCACAGCCAGGT | 4592 | Synthesized from |
| | | | Millipore-Sigma |
| sgCYLD#1 | AAAGCATGTTTTCAGCAACG | 4593 | Synthesized from |
| | | | Millipore-Sigma |
| sgCYLD#2 | TTCAACTAAGGAACTCCGGG | 4594 | Synthesized from |
| | | | Millipore-Sigma |
| sgSPATA2#1 | TCAACCTCTTCCTCTACCCG | 4595 | Synthesized from |
| | | | Millipore-Sigma |
| sgSPATA2#2 | CAGATCCACGTCATCCTGAG | 4596 | Synthesized from |
| | | | Millipore-Sigma |
| sgGSDMD#1 | AGGTTGACACACTTATAACG | 4597 | Synthesized from |
| | | | Millipore-Sigma |
| sgGSDMD#2 | CAGGGATGAACTCCCCACCA | 4598 | Synthesized from |
| | | | Millipore-Sigma |
| sgNLRP3 | GATCGCAGCGAAGATCCACA | 4601 | Synthesized from |
| | | | Millipore-Sigma |
| shPTPN1 | CCGCCCAAAGGAGTTACATTC | 4599 | Synthesized from |
| | | | Millipore-Sigma |
| shPtpn1 | CCGCCCAGAGGAGCTATATTC | 4600 | Synthesized from |
| | | | Millipore-Sigma |
| siABL1 | — | — | ON-TARGETplus |
| | | | SMARTpool Human |
| | | | ABL1 siRNA cat. no. L- |
| | | | 003100-00-0005 |
| siABL2 | — | — | ON-TARGETplus |
| | | | SMARTpool Human |
| | | | ABL2 siRNA cat. no. L- |
| | | | 003101-00-0005 |
| siRBCK1 | — | — | ON-TARGETplus |
| | | | SMARTpool Human |
| | | | RBCKI siRNA cat. |
| | | | no. L-006932-00-0005 |
| siRNF31 | — | — | ON-TARGETplus |
| | | | SMARTpool Human |
| | | | RNF31 siRNA cat. no. |
| | | | L-021419-00-0005 |
| siDDIT3 | — | — | ON-TARGETplus |
| | | | SMARTpool Human |
| | | | DDIT3 siRNA cat. no. |
| | | | L-004819-00-0005 |
| siSPATA2 | — | — | ON-TARGETplus |
| | | | SMARTpool Human |
| | | | SPATA2 siRNA cat. no. |
| | | | L-020065-02-0005 |

Piggbac (PB)-based transposon expression was used to re-express WT RNF213 or RNF213 variants under Dox control in RNF213-KO BT474 and RNF213-KO AU565 cells. Briefly, PB-TA-ERN (Addgene)-based expression constructs containing RNF213 and RNF213 variants were co-transfected with transposase expression vector, and integrants were selected by adding G418 (800 μg/ml). RNF213 expression was induced by adding 0.5 gig/ml Dox.

PTPN1 depletion was accomplished by stable shRNA knockdown using pSUPER-Puro-based retroviral vectors. RBCK1, ABL1, and ABL2 siRNA (Horizon Discovery) were introduced using Lipofectamine™ RNAiMAX reagent (ThermoFisher Scientific). Pooled knockout lines were generated by infection with lentiviruses expressing CAS9 and appropriate sgRNAs. Lentiviruses and retroviruses were packaged as described⁷¹.

### Viability Assays

Cells were seeded in 6-well plates (2×10⁶/well for MDAMB361, 1.5×10⁶/well for other lines) and maintained in at 37° C., 500 CO₂. After 24 hours, viable cell number was determined by Trypan Blue exclusion, and fresh media was added to replicates placed in a standard incubator (normoxia) and in hypoxic conditions (0.1% O₂, Whitley H35 Hypoxystation). Viable cells were counted at various times, as indicated.

### Generation of RNF213 Mutants

RNF213 mutants were generated by PCR or by using fragments synthesized by Genewiz/IDT. Fragments containing the desired mutation were sequenced, digested, and cloned into wild-type RNF213 plasmids by using T4 DNA ligase or via Gibson assembly⁷².

### NF-κB Reporter Assays

Cells co-transfected with an NF-κB reporter construct (p2×-NF-κB-BS-Luc-Firefly, a generous gift of Dan Littman, NYU Grossman School of Medicine) and pCMV-Renilla, were subjected to 0.1% O₂ for the indicated times. Firefly and Renilla luciferase was measured using the Dual-Luciferase® Reporter Assay System (Promega) according to the manufacturer's instructions. Reporter activity was quantified as the ratio of Firefly/Renilla signal, and all signals were normalized to the signal in parental BT474 cells.

### IL-1β Assays

Cell supernatants were collected at the indicated times, and IL-1 levels were quantified by using the Lumit™ IL-10 Immunoassay (Promega), according to manufacturer's instructions.

### Biochemical Assays

### Immunoblotting and Immunoprecipitations

Cells were lysed in RIPA (total cell lysates, ALFA-tag precipitations) or NP40 buffer (co-immunoprecipitations); buffer compositions and immunoprecipitation conditions are described below²⁷. ALFA-tagged proteins were recovered using a single domain camelid antibody bound to magnetic beads (NanoTag Biotechnologies). Antibody details and concentrations used are summarized in Table 3.

TABLE 3
Antibodies and related reagents

| Antibodies against | | | |
|---|---|---|---|
| protein used | Vendor | Catalog | Dilution |
| RNF213 | ThermoFisher Scientific | # PA5-51902 | 1:1000 |
| RNF213 | Millipore Sigma | ABC1391 | 1:1000 |
| PTP1B | Biotechne-RnD Systems | AF1366 | 1:1000 |
| HA | Cell Signaling Technology | 3724S | 1:1000 |
| FLAG | Millipore Sigma | F1804-1MG | 1:2000 |
| CYLD | Cell Signaling Technology | 8462S | 1:1000 |
| SPATA2 | Santacruz | sc-515283 | 1:500 |
| ERK2 | Santacruz | sc-1647 | 1:2000 |
| Vinculin | Santacruz | sc-73614 | 1:2000 |
| GSDMD | Cell Signaling Technology | 39754S | 1:1000 |
| V5 | Cell Signaling Technology | 1320S | 1:1000 |
| ALFA | NanoTag Biotechnologies | N1582 | 1:1000 |
| pY | Millipore Sigma | 05-321 | 1:1000 |
| ABL1 | Santacruz | sc-56887 | 1:1000 |
| ABL2 | ThermoFisher Scientific | XD3571172 | 1:1000 |
| V5 | Santacruz | sc-271944 | 1:1000 |
| GFP | Cell Signaling Technology | 2555S | 1:1000 |
| Cleaved Caspase1 | Cell Signaling Technology | 4199 | 1:1000 |
| Cleaved GSDMD | Cell Signaling Technology | 36425 | 1:1000 |
| RIPK3/RIP3 | Fisher Scientific | MAB7604SP | 1:1000 |
| Antibody | | | |
| pMLKL | Millipore Sigma | 91689 | 1:1000 |
| Cleaved Caspase3 | Cell Signaling Technology | 9661 | 1:1000 |
| EF5, Alexa Fluor ® | Millipore Sigma | EF5010-250UG | — |
| 488 conjugate | | | |
| Ki67 | NYU Pathology Core | | — |
| HIF-1a | NYU Pathology Core | | — |
| Beads | Vendor | Catalog | — |
| Streptavidin beads | Fisher Scientific | PI20357 | — |
| FLAG M2 beads | Millipore Sigma | MM823 | — |
| HA-beads | ThermoFisher Scientific | 26181 | — |
| ALFA-beads | NanoTag Biotechnologies | N1511 | — |
| Glutathione | Cytiva | 17075601 | — |
| Sepharose 4B beads | | | |

### Proximity-Based Labelling

Cells were cultured in standard media but with 10% tetracycline (Tet)-free FBS (Clonetech) for at least 1 week. Tet-free/biotin-free FBS was generated by incubating Tet-free FBS with sterile streptavidin beads overnight followed by centrifugation to remove the beads. N-Turbo, C-Turbo, and Turbo proteins were induced by adding 1 μg/ml Dox to cells for 36 hours. For the last ten min. of incubation, 500 μM of biotin was added to the medium. Labelling reactions was terminated by removing the medium and adding ice-cold PBS to plates, which were kept on ice. Lysates were collected in RIPA buffer without Na-deoxycholate, and biotin-labelled proteins were collected by incubating with high-capacity streptavidin agarose beads for 4 hours at 4° C. Comparable protein amounts (assessed by silver stain and avidin blotting) were analyzed by MS.

### Purification of RNF213 and RNF213 Variants

HeLa RNF213-KO cells expressing RNF213 or RNF213 variants (except RNF213^{H4509A}) were induced with 0.5 μg/ml Dox for 4 days. The 3×-FLAG-RNF213^{H4509A} variant was transiently transfected into HeLa RNF213-KO cells, as the stable expression of this variant was low. Lysates from ten 15 cm plates were subjected to anti-FLAG immunoprecipitation. RNF213 species were eluted with 3×-FLAG peptide and concentrated by passage through an Amicon Ultra-4 Centrifugal Filter (100 kDa cut-off).

### In Vitro Ubiquitylation Assays

Ubiquitylation assays were performed as described²⁷. Briefly, 0.25 μM of purified RNF213 or RNF213 variant were incubated with 2.5 μM GST-SPATA2 (Abnova) or 2.5 μM 6×His-CYLD (R&D) in E3-Ligase buffer (R&D) along with 50 μM HA-Ub, 100 nM UBE1 (E1), 1 μM UBE2L3 (E2), and 10 mM Mg-ATP (all from Boston Biochem) for 30 min. in a total reaction volume of 25 μl. Reactions were terminated by adding SDS-PAGE sample buffer followed by incubation for 5 min. at 95° C.

### In Vitro Kinase Assays

Purified full-length 3×-FLAG-tagged RNF213 or RNF213^{Y1275F} were mixed with varying amounts of bacterially produced His-ABL1 (as indicated) and incubated with 600 ng of RNF213 in kinase assay buffer (50 mM HEPES [pH 7.5], 10 mM MgCl₂, 2.5 mM DTT, 75 μM ATP) for varying times, as indicated. Final reaction volumes were 20 μl. Reactions were terminated by adding SDS-PAGE sample buffer followed by incubation for 5 min. at 95° C.

### Mass Spectrometry

Proteins collected on streptavidin beads were digested with sequencing grade trypsin and analyzed by LC/MS/MS using a Thermo Scientific EASY-nLC 1200 and a Thermo Scientific Orbitrap Eclipse Mass Spectrometer MS/MS spectra were searched against the Uniprot human database with the addition of the sequences of RNF213 with TurboID at the N- and C-terminus using Sequest within Proteome Discoverer 1.4. MS/MS spectra were also searched using Byonic by Protein Metrics specifically to identify phosphorylation sites on RNF213.

### Quantification and Statistical Analysis

Graphs were generated and statistical analyses were performed using GraphPad Prism9. Multiple groups were compared by two-way ANOVA, with additional Tukey multiple comparisons test. Two group comparisons were evaluated by two-tailed t test. Values represent the mean of experiments, with error bars representing S.E.M.

### Immunoblotting, Immunoprecipitation, and SDS-PAGE

For co-immunoprecipitations, cells were lysed in NP40 buffer (50 mM Tris-HCl [pH 8], 150 mM NaCl, 2 mM EDTA, 10 mM Na₄P₂O₇, 100 mM NaF, 2 mM Na₃VO₄, 1% [vol/vol] NP-40, 40 μg/ml phenylmethyl sulfonyl fluoride, 2 μg/ml antipain, 2 μg/ml pepstatin A, 20 μg/ml leupeptin, and 20 μg/ml aprotinin). For streptavidin affinity purifications and standard immunoprecipitations, modified RIPA buffer (50 mM Tris-HCl [pH 8], 150 mM NaCl, 2 mM EDTA, 10 mM Na₄P₂O₇, 100 mM NaF, 2 mM Na₃VO₄, 10% [vol/vol] NP-40, 0.10% (wt/vol) SDS, 40 μg/ml phenylmethyl sulfonyl fluoride, 2 μg/ml antipain, 2 μg/ml pepstatin A, 20 μg/ml leupeptin, and 20 μg/ml aprotinin) was used for cell lysis. For detection of ubiquitylated species in vivo, 10 mM N-ethylmaleimide (NEM) and 10 mM iodoacetamide (IAM) were added to the lysis buffer.

For immunoblotting of whole cell lysates, equal amounts of protein per sample were subjected to SDS-PAGE and transferred to polyvinylidene fluoride (PVDF) membranes (Millipore). For immunoprecipitation of FLAG-tagged proteins, lysates were incubated with anti-FLAG-magnetic beads for 4 hours on a rotator-mixer at 4° C. For immunoprecipitation of HA-tagged proteins, lysates were incubated with anti-HA-agarose beads for 4 hours on a rotator-mixer at 4° C. For immunoprecipitation of ALFA-tagged proteins, lysates were incubated with ALFA-selector ST agarose beads overnight on a rotor-mixer at 4° C. For recovery of biotinylated proteins, high-capacity streptavidin agarose resin (ThermoScientific) was added to lysates, which were rotated for 4 hours at 4° C. Beads were washed five times in lysis buffer, incubated in sample buffer at 95C for 5 min. and resolved by SDS-PAGE and immunoblotting. For in vitro ubiquitylation and phosphorylation using purified RNF213 and RNF213 variants, proteins were eluted from beads by incubation with an excess of 3×-FLAG peptide. Lysates and immunoprecipitates were resolved by modified SDS-PAGE on 3-8% Tris-acetate gels (Novex) or 5% Tris-glycine gels. Gels were transferred in 1× transfer buffer, 10% methanol for 1 hour at 25 V using an XCell II Blot Module (Novex) and analyzed by acquisition software (Image Studio Lite), on an Odyssey Infrared imaging system (Li-Cor Biosciences). All antibodies used are listed in Table 3 above.

### Mass Spectrometry

Proteins bound to streptavidin agarose beads were washed with 100 mM NH₄HCO₃ (pH 8), reduced with 2.5 μL of 0.2M dithiothreitol at 57C for 1 hour, alkylated with 2.5 μL of 0.5M IAM for 45 min. at room temperature in the dark, and digested overnight at room temperature with 500 ng sequencing grade modified trypsin (Promega). After digestion, samples were acidified to a final concentration of 0.5% trifluoroacetic acid (TFA). A C18 cleanup was performed on all samples using Ultra-Micro SpinColumns (Harvard Apparatus). Briefly, samples were loaded onto equilibrated spin columns, rinsed with 0.1% TFA, and eluted with 40% acetonitrile/0.5% acetic acid and 80% acetonitrile/0.5% acetic acid. Organic solvent was removed using a SpeedVac concentrator, and all samples were reconstituted in 0.5% acetic acid.

One-third of each sample was analyzed individually by LC/MS/MS. Samples were separated online using a Thermo Scientific EASY-nLC 1200, where solvent A was 2% acetonitrile/0.5% acetic acid, and solvent B was 80% acetonitrile/0.5% acetic acid. A 60-min. gradient from 5-35% B was applied to all samples. Peptides were gradient-eluted directly to a Thermo Scientific Orbitrap Eclipse Mass Spectrometer. High resolution, full MS spectra were acquired with a resolution of 240,000, an AGC target of 1e6, a maximum ion time of 50 ms, and a scan range of 400 to 1500 m/z. Following each full MS scan, data dependent HCD MS/MS spectra collected with low resolution at top speed with a 3 ms cycle time. All MS/MS spectra were collected with a rapid scan in the ion trap, an AGC target of 2e4, a maximum ion time of 18 ms, one microscan, 0.7 m/z isolation window, auto scan range mode, and NCE of 27. MS/MS spectra were searched against the Uniprot human database using Sequest within Proteome Discoverer. 1.4. MS/MS spectra were also searched with Byonic by Protein Metrics against a database containing just RNF213 and common contaminants to identify phosphorylation sites.

For TurboID analyses, beads were washed two times with 100 mM NH₄HCO (pH 8). Samples were reduced with 2 μL of 0.2M dithiothreitol at 57C for 1 hour and alkylated with 2 μL of 0.5M IAM for 45 min. at room temperature in the dark. Samples were digested overnight at room temperature with 250 ng sequencing trypsin. After digestion, samples were acidified to a final concentration of 0.5% trifluoroacetic acid (TFA) and cleaned up on C18 columns as above.

One-third of each sample was analyzed individually by LC/MS/MS. LC conditions were identical to those above. High resolution full MS spectra were acquired with a resolution of 70,000, an AGC target of 1e6, a maximum ion time of 120 ms, and scan range of 400 to 1500 m/z. Following each full MS were 20 data-dependent high resolution HCD MS/MS spectra. All MS/MS spectra were collected with resolution of 17,500, an AGC target of 5e4, a maximum ion time of 120 ms, one microscan, 2 m/z isolation window, fixed first mass of 150 m/z, and NCE of 27. MS/MS spectra were searched against the Uniprot human database with the added sequences of RNF213 with the TurboID on the N- and C-terminus using Sequest within Proteome Discoverer 1.4.

Additional chemicals, kits, and reagents used in above-described methods are provided in Table 4. Examples of plasmids used in the above-described methods are provided in Table 5A. Examples of primers used in the above-described methods are provided in Table 5B.

TABLE 4
Chemicals and kits

| Chemical | Vendor | Catalog |
|---|---|---|
| Claramine trifuoroacetate salt | Millipore Sigma | SML1545-25MG |
| Crizotinib | MedChemExpress | HY-50878 |
| FAK inhibitor 14 | Millipore Sigma | SML0837-10MG |
| PP2 | MedChemExpress | HY-13805 |
| Saracatinib | Selleck | S1006 |
| Dasatinib | Selleck | S1021 |
| Asciminib | Selleck | S8555 |
| Biotin | Millipore Sigma | B4501 |
| Z-VAD-FMK | Selleck | S7023 |
| Ferrostatin-1 | Selleck | S7243 |
| Necrostatin | ThermoFisher | 23-241-0 |
| VX765 | Millipore Sigma | 5313720001 |
| MCC950 | Millipore Sigma | 5381200001 |
| 48 μC | MedChemExpress | HY-19701 |
| GSK2606414 | MedChemExpress | HY-18072 |
| Tunicamycin | Tocris Biosciences | 3516 |
| Thapsigargin | Cayman | 10522-5mg |
| Iodoacetamide | Millipore Sigma | I1149-5G |
| N-Ethylmaleimide | Millipore Sigma | 04259-5G |
| SM-164 | MedChemExpress | HY-15989 |
| EF5 | Millipore Sigma | SML1961 |
| Cyclohexamide | Millipore Sigma | C1988 |
| MG132 | Millipore Sigma | M7449 |
| Chloroquine | Millipore Sigma | C6628 |
| BODIPY ™ 581/591 C11 | ThermoFisher | D3861 |
| Nigericin sodium salt | Millipore Sigma | N7143-5MG |
| Doxocrubicin | Millipore Sigma | D5220 |
| Kit | Vendor | Catalog |
| Dual-Luciferase Reporter Assay System | Promega | E1910 |
| Lumit ™ Human IL-1beta immunoassay | Promega | W6010 |
| LDH-Glo ™ Cytotoxicity Assay | Promega | J2380 |
| Click-iT ™ EdU Cell Proliferation Kit for | ThermoFisher | C10340 |
| Imaging | | |
| Ni-NTA Spin Kit | Qiagen | 31314 |
| Reagent | Vendor | Catalog |
| His-ABL1 | Abcam | ab69810 |
| His-CYLD | R&D | E-556 |
| GST-SPATA2 | Abnova | H00009825-P01 |
| TNF-alpha/TNFSF2 MEDCHEM | MedChemExpress | HY-P7058 |
| Gateway ™ BP Clonase ™ II Enzyme mix | ThermoFisher | 11789020 |
| | Scientific | |
| Gateway ™ LR Clonase ™ II Enzyme mix | ThermoFisher | 11791020 |
| | Scientific | |

TABLE 5A
Plasmids

| Plasmids | Source |
|---|---|
| ALFA-SPATA2 | in this study |
| ALFA-CYLD | in this study |
| HA-Ptpn1-DA | in this study |
| pcDNA5-3x-FLAG-RNF213 | Used in Bhardwaj et al., 2022 |
| pcDNA5-3x-FLAG-RNF213^{Y1275F} | in this study |
| pcDNA5-3x-FLAG-RNF213^{H4014A} | Used in Bhardwaj et al., 2022 |
| pcDNA5-3x-FLAG-RNF213^{H4509A} | in this study |
| pcDNA5-3x-FLAG- | in this study |
| RNF213^{H4014A/H4509A} | |
| pcDNA5-GFP-RNF213 | in this study |
| pcDNA5-3x-FLAG-RNF213-TurboID | in this study |
| pcDNA5-TurboID-RNF213 | in this study |
| pcDNA5-V5-TurboID | in this study |
| pcDNA5-BirA | in this study |
| pcDNA5-BirA-RNF213 | in this study |
| PB-TA-ERN-RNF213 | in this study |
| PB-TA-ERN-RNF213^{Y1275F} | in this study |
| PB-TA-ERN-RNF213^{K2775A} | in this study |
| PB-TA-ERN-RNF213^{C3997Y} | in this study |
| PB-TA-ERN-RNF213^{H4014A} | in this study |
| PB-TA-ERN-RNF213^{H4509A} | in this study |
| PB-TA-ERN-RNF213^{R4810K} | in this study |
| PB-TA-ERN-RNF213^{H4014A/H4509A} | in this study |
| PB-TA-ERN-RNF213^{K2775A/H4014A} | in this study |
| p2X-NF-kB-BS-Luc-Firefly | NYU |
| pCMV-Renilla | NYU |
| HA-UB | Used in Bhardwaj et al., 2022 |

TABLE 5B
Primers

| Gene | Forward primer | SEQ ID | Reverse primer | SEQ ID | |
|---|---|---|---|---|---|
| Name | (5′->3′) | NO | (5′->3′) | NO | Vendor |
| IL1B | CAGAAGTACC | 4602 | GAAGCCCTTG | 4611 | Millipore Sigma |
| | TGAGCTCGCC | | CTGTAGTGGT | | |
| NLRP3 | GATCTTCGCTG | 4603 | GACGGGCATT | 4612 | Millipore Sigma |
| | CGATCAACA | | CCTGTCTTCA | | |
| DDIT3 | TTAAAGATGA | 4604 | ATTTTTGGAA | 4613 | Millipore Sigma |
| | GCGGGTGGCA | | AAGGGTAGG | | |
| | | | TTAAGT | | |
| HSPA5 | CACTCCTGAA | 4605 | ACCACCTTGA | 4614 | Millipore Sigma |
| | GGGGAACGTC | | ACGGCAAGA | | |
| | | | A | | |
| ERN1 | ACCAGCGTGG | 4606 | CTGGAATTGC | 4615 | Millipore Sigma |
| | TGATAGTTGG | | CGGTCCTTCT | | |
| CYLD | GTCCTGGGGA | 4607 | TCACACTCTC | 4616 | Millipore Sigma |
| | CACAATGCAG | | TGATAAAGCT | | |
| | | | GGG | | |
| SPATA2 | GATGGCTTTG | 4608 | TCCATTGAAC | 4617 | Millipore Sigma |
| | CAAGAGTCGG | | TGGGCTTCCC | | |
| RBCK1 | CAGGTGGCAA | 4609 | TAGAGGTAG | 4618 | Millipore Sigma |
| | TGAAGTGTGC | | GCACTGTCCC | | |
| | | | C | | |
| RNF31 | GAGTGACCGT | 4610 | TGTTGAGGTA | 4619 | Millipore Sigma |
| | GGTCTGAGTG | | GTTTCGGGGC | | |

### REFERENCES

1. Bertout, J. A., Patel, S. A. & Simon, M. C. The impact of 02 availability on human cancer. Nat Rev Cancer 8, 967-975 (2008). doi.or2:10.1038/nrc2540
2. Schofield, C. J. & Ratcliffe, P. J. Oxygen sensing by HIF hydroxylases. Nat Rev Mol Cell Biol 5, 343-354 (2004). doi.org:10.1038/nrm1366
3. Singleton, D. C., Macann, A. & Wilson, W. R. Therapeutic targeting of the hypoxic tumour microenvironment. Nat Rev Clin Oncol 18, 751-772 (2021). doi.org:10.1038/s41571-021-00539-4
4. Wouters, B. G. & Koritzinsky, M. Hypoxia signalling through mTOR and the unfolded protein response in cancer. Nat Rev Cancer 8, 851-864 (2008). doi.org:10.1038/nrc2501
5. Cummins, E. P. et al. Prolyl hydroxylase-1 negatively regulates IkappaB kinase-beta, giving insight into hypoxia-induced NFkappaB activity. Proc Natl Acad Sci USA 103, 18154-18159 (2006). doi.or2:10.1073/pnas.0602235103
6. Rius, J. et al. NF-kappaB links innate immunity to the hypoxic response through transcriptional regulation of HIF-1alpha. Nature 453, 807-811 (2008). doi.org:10.1038/nature06905
7. Eltzschig, H. K., Bratton, D. L. & Colgan, S. P. Targeting hypoxia signalling for the treatment of ischaemic and inflammatory diseases. Nat Rev Drug Discov 13, 852-869 (2014). doi.org:10.1038/nrd4422
8. Nizet, V. & Johnson, R. S. Interdependence of hypoxic and innate immune responses. Nat Rev Immunol 9, 609-617 (2009). doi.org:10.1038/nri2607
9. Pakos-Zebrucka, K. et al. The integrated stress response. EMBO Rep 17, 1374-1395 (2016). doi.org:10.15252/embr.201642195
10. Kroemer, G., Galluzzi, L., Kepp, O. & Zitvogel, L. Immunogenic cell death in cancer therapy. Annu Rev Immunol 31, 51-72 (2013). doi.or2:10.1146/annurev-immunol-032712-100008
11. Eltzschig, H. K. & Carmeliet, P. Hypoxia and inflammation. N Engl J Med 364, 656-665 (2011). doi.org:10.1056/NEJMra0910283
12. Frangioni, J. V., Beahm, P. H., Shifrin, V., Jost, C. A. & Neel, B. G. The nontransmembrane tyrosine phosphatase PTP-1B localizes to the endoplasmic reticulum via its 35 amino acid C-terminal sequence. Cell 68, 545-560 (1992). doi.org:10.1016/0092-8674(92)90190-n
13. Haj, F. G., Verveer, P. J., Squire, A., Neel, B. G. & Bastiaens, P. I. Imaging sites of receptor dephosphorylation by PTP1B on the surface of the endoplasmic reticulum. Science 295, 1708-1711 (2002). doi.org:10.1126/science.1067566
14. Elchebly, M. et al. Increased insulin sensitivity and obesity resistance in mice lacking the protein tyrosine phosphatase-1B gene. Science 283, 1544-1548 (1999). doi.org:10.1126/science.283.5407.1544
15. Klaman, L. D. et al. Increased energy expenditure, decreased adiposity, and tissue-specific insulin sensitivity in protein-tyrosine phosphatase 1B-deficient mice. Mol Cell Biol 20, 5479-5489 (2000). doi.org:10.1128/MCB.20.15.5479-5489.2000
16. Zabolotny, J. M. et al. PTP1B regulates leptin signal transduction in vivo. Dev Cell 2, 489-495 (2002). doi.org:10.1016/si534-5807(02)00148-x
17. Cheng, A. et al. Attenuation of leptin action and regulation of obesity by protein tyrosine phosphatase 1B. Dev Cell 2, 497-503 (2002). doi.org:10.1016/s1534-5807(02)00149-1
18. Bentires-Alj, M. & Neel, B. G. Protein-tyrosine phosphatase 1B is required for HER2/Neu-induced breast cancer. Cancer Res 67, 2420-2424 (2007). doi.org:10.1158/0008-5472.CAN-06-4610
19. Julien, S. G. et al. Protein tyrosine phosphatase 1B deficiency or inhibition delays ErbB2-induced mammary tumorigenesis and protects from lung metastasis. Nat Genet 39, 338-346 (2007). doi.org:10.1038/ng1963
20. Cancer Genome Atlas, N. Comprehensive molecular portraits of human breast tumours. Nature 490, 61-70 (2012). doi.org:10.1038/nature11412
21. Wiener, J. R. et al. Overexpression of the protein tyrosine phosphatase PTP1B in human breast cancer: association with p185c-erbB-2 protein expression. J Natl Cancer Inst 86, 372-378 (1994). doi.or2:10.1093/jnci/86.5.372
22. Banh, R. S. et al. PTP1B controls non-mitochondrial oxygen consumption by regulating RNF213 to promote tumour survival during hypoxia. Nat Cell Biol 18, 803-813 (2016). doi.org:10.1038/ncb3376
23. Kamada, F. et al. A genome-wide association study identifies RNF213 as the first Moyamoya disease gene. J Hum Genet 56, 34-40 (2011). doi.org:10.1038/jh2.2010.132
24. Nanba, R. et al. [Familial moyamoya disease--clinical features and current study]. No Shinkei Geka 32, 7-16 (2004).
25. Nanba, R. et al. Clinical features of familial moyamoya disease. Childs Nerv Syst 22, 258-262 (2006). doi.org:10.1007/s00381-005-1230-5
26. Ahel, J. et al. Moyamoya disease factor RNF213 is a giant E3 ligase with a dynein-like core and a distinct ubiquitin-transfer mechanism. Elife 9 (2020). doi.org:10.7554/eLife.56185
27. Bhardwaj, A., Banh, R. S., Zhang, W., Sidhu, S. S. & Neel, B. G. MMD-associated RNF213 SNPs encode dominant-negative alleles that globally impair ubiquitylation. Life Sci Alliance 5 (2022). doi.org:10.26508/lsa.202000807
28. Morito, D. et al. Moyamoya disease-associated protein mysterin/RNF213 is a novel AAA+ ATPase, which dynamically changes its oligomeric state. Sci Rep 4, 4442 (2014). doi.org:10.1038/srep04442
29. Otten, E. G. et al. Ubiquitylation of lipopolysaccharide by RNF213 during bacterial infection. Nature 594, 111-116 (2021). doi.or2:10.1038/s41586-021-03566-4
30. Takeda, M. et al. Moyamoya disease patient mutations in the RING domain of RNF213 reduce its ubiquitin ligase activity and enhance NFkappaB activation and apoptosis in an AAA+ domain-dependent manner. Biochem Biophys Res Commun 525, 668-674 (2020). doi.org:10.1016/j.bbrc.2020.02.024
31. Sugihara, M. et al. The AAA+ ATPase/ubiquitin ligase mysterin stabilizes cytoplasmic lipid droplets. J Cell Biol 218, 949-960 (2019). doi.org:10.1083/jcb.201712120
32. Piccolis, M. et al. Probing the Global Cellular Responses to Lipotoxicity Caused by Saturated Fatty Acids. Mol Cell 74, 32-44 e38 (2019). doi.org:10.1016/j.molcel.2019.01.036
33. Habu, T. & Harada, K. H. UBC13 is an RNF213-associated E2 ubiquitin-conjugating enzyme, and Lysine 63-linked ubiquitination by the RNF213-UBC13 axis is responsible for angiogenic activity. FASEB Bioadv 3, 243-258 (2021). doi.org:10.1096/fba.2019-00092
34. Baumgartner, C. K. et al. ABBV-CLS-484: An active site PTPN2/N1 inhibitor that augments the immune response and sensitizes tumors to immune-mediated killing. Cancer Research 82 (2022).
35. Branon, T. C. et al. Efficient proximity labeling in living cells and organisms with TurboID. Nat Biotechnol 36, 880-887 (2018). doi.org:10.1038/nbt.4201
36. Teo, G. et al. SAINTexpress: improvements and additional features in Significance Analysis of INTeractome software. J Proteomics 100, 37-43 (2014). doi.org:10.1016/j.jprot.2013.10.023
37. Kotani, Y. et al. Alternative exon skipping biases substrate preference of the deubiquitylase USP15 for mysterin/RNF213, the moyamoya disease susceptibility factor. Sci Rep 7, 44293 (2017). doi.or2:10.1038/srep44293
38. Louvrier, C. et al. RNF213-associated urticarial lesions with hypercytokinemia. J Allergy Clin Immunol 150, 1545-1555 (2022). doi.org:10.1016/j.jaci.2022.06.016
39. Ruzehaji, N. et al. Attenuation of flightless I improves wound healing and enhances angiogenesis in a murine model of type 1 diabetes. Diabetologia 57, 402-412 (2014). doi.org:10.1007/s00125-013-3107-6
40. Bematchez, P. N. et al. Myoferlin regulates vascular endothelial growth factor receptor-2 stability and function. J Biol Chem 282, 30745-30753 (2007). doi.org:10.1074/jbc.M704798200
41. Roth Flach, R. J. et al. Endothelial protein kinase MAP4K4 promotes vascular inflammation and atherosclerosis. Nat Commun 6, 8995 (2015). doi.org:10.1038/ncomms9995
42. Tamehiro, N. et al. SPTLC1 binds ABCA1 to negatively regulate trafficking and cholesterol efflux activity of the transporter. Biochemistry 47, 6138-6147 (2008). doi.org:10.1021/bi800182t
43. Chaurasia, B. & Summers, S. A. Ceramides in Metabolism: Key Lipotoxic Players. Annu Rev Physiol 83, 303-330 (2021). doi.org:10.1146/annurev-physiol-031620-093815
44. Chen, X. et al. A noncanonical function of EIF4E limits ALDH1B1 activity and increases susceptibility to ferroptosis. Nat Commun 13, 6318 (2022). doi.or2:10.1038/s41467-022-34096-w
45. Kan, M. C. et al. CPEB4 is a cell survival protein retained in the nucleus upon ischemia or endoplasmic reticulum calcium depletion. Mol Cell Biol 30, 5658-5671 (2010). doi.or2:10.1128/MCB.00716-10
46. Roux, C. J. et al. Phenotypic diversity of brain MRI patterns in mitochondrial aminoacyl-tRNA synthetase mutations. Mol Genet Metab 133, 222-229 (2021). doi.org:10.1016/j.vmgme.2021.04.004
47. Rozman, D. Lanosterol 14alpha-demethylase (CYP51)—a cholesterol biosynthetic enzyme involved in production of meiosis activating sterols in oocytes and testis—a minireview. Pflugers Arch 439, r056-r057 (2000). doi.org:10.1007/s004240000090
48. Liu, W. et al. Identification of RNF213 as a susceptibility gene for moyamoya disease and its possible role in vascular development. PLoS One 6, e22542 (2011). doi.org:10.1371/journal.pone.0022542
49. Cecchi, A. C. et al. RNF213 rare variants in an ethnically diverse population with Moyamoya disease. Stroke 45, 3200-3207 (2014). doi.org:10.1161/STROKEAHA.114.006244
50. Moquin, D. M., McQuade, T. & Chan, F. K. CYLD deubiquitinates RIP1 in the TNFalpha-induced necrosome to facilitate kinase activation and programmed necrosis. PLoS One 8, e76841 (2013). doi.org:10.1371/journal.pone.0076841
51. Kovalenko, A. et al. The tumour suppressor CYLD negatively regulates NF-kappaB signalling by deubiquitination. Nature 424, 801-805 (2003). doi.org:10.1038/nature01802
52. Taniguchi, K. & Karin, M. NF-kappaB, inflammation, immunity and cancer: coming of age. Nat Rev Immunol 18, 309-324 (2018). doi.org:10.1038/nri.2017.142
53. Karki, R. & Kanneganti, T. D. Diverging inflammasome signals in tumorigenesis and potential targeting. Nat Rev Cancer 19, 197-214 (2019). doi.org:10.1038/s41568-019-0123-y
54. Menu, P. et al. ER stress activates the NLRP3 inflammasome via an UPR-independent pathway. Cell Death Dis 3, e261 (2012). doi.org:10.1038/cddis.2011.132
55. Dong, J. et al. Small Molecule Degraders of Protein Tyrosine Phosphatase 1B and T-Cell Protein Tyrosine Phosphatase for Cancer Immunotherapy. Angew Chem Int Ed Engl 62, e202303818 (2023). doi.org:10.1002/anie.202303818
56. Scholz, C. C. et al. Regulation of IL-1beta-induced NF-kappaB by hydroxylases links key hypoxic and inflammatory signaling pathways. Proc Natl Acad Sci USA 110, 18490-18495 (2013). doi.org:10.1073/pnas.1309718110
57. An, J. et al. Inactivation of the CYLD deubiquitinase by HPV E6 mediates hypoxia-induced NF-kappaB activation. Cancer Cell 14, 394-407 (2008). doi.org:10.1016/j.ccr.2008.10.007
58. Jura, N., Archer, H. & Bar-Sagi, D. Chronic pancreatitis, pancreatic adenocarcinoma and the black box in-between. Cell Res 15, 72-77 (2005). doi.org:10.1038/sj.cr.7290269
59. Grant, L. et al. Myeloid-cell protein tyrosine phosphatase-1B deficiency in mice protects against high-fat diet and lipopolysaccharide-induced inflammation, hyperinsulinemia, and endotoxemia through an IL-10 STAT3-dependent mechanism. Diabetes 63, 456-470 (2014). doi.org:10.2337/db13-0885
60. Wiede, F. et al. PTP1B Is an Intracellular Checkpoint that Limits T-cell and CAR T-cell Antitumor Immunity. Cancer Discov 12, 752-773 (2022). doi.or2:10.1158/2159-8290.CD-21-0694
61. Medgyesi, D. et al. The protein tyrosine phosphatase PTP1B is a negative regulator of CD40 and BAFF-R signaling and controls B cell autoimmunity. J Exp Med 211, 427-440 (2014). doi.org:10.1084/jem.20131196
62. Stanford, S. M. & Bottini, N. Targeting protein phosphatases in cancer immunotherapy and autoimmune disorders. Nat Rev Drug Discov 22, 273-294 (2023). doi.org:10.1038/s41573-022-00618-w
63. Chen, X., Zeh, H. J., Kang, R., Kroemer, G. & Tang, D. Cell death in pancreatic cancer: from pathogenesis to therapy. Nat Rev Gastroenterol Hepatol 18, 804-823 (2021). doi.org:10.1038/s41575-021-00486-6
64. Thery, F. et al. Ring finger protein 213 assembles into a sensor for ISGylated proteins with antimicrobial activity. Nat Commun 12, 5772 (2021). doi.org:10.1038/s41467-021-26061-w
65. Ohkubo, K. et al. Moyamoya disease susceptibility gene RNF213 links inflammatory and angiogenic signals in endothelial cells. Sci Rep 5, 13191 (2015). doi.or2:10.1038/srep13191
66. Asselman, C., Hemelsoet, D., Eggermont, D., Dermaut, B. & Impens, F. Moyamoya disease emerging as an immune-related angiopathy. Trends Mol Med 28, 939-950 (2022). doi.org:10.1016/j.molmed.2022.08.009
67. Karasawa, T. et al. Saturated Fatty Acids Undergo Intracellular Crystallization and Activate the NLRP3 Inflammasome in Macrophages. Arterioscler Thromb Vasc Biol 38, 744-756 (2018). doi.or2:10.1161/ATVBAHA.117.310581
68. Wang, D., Wei, Y. & Pagliassotti, M. J. Saturated fatty acids promote endoplasmic reticulum stress and liver injury in rats with hepatic steatosis. Endocrinology 147, 943-951 (2006). doi.org:10.1210/en.2005-0570
69. Marcotte, R. et al. Functional Genomic Landscape of Human Breast Cancer Drivers, Vulnerabilities, and Resistance. Cell 164, 293-309 (2016). doi.org:10.1016/j.cell.2015.11.062
70. Ran, F. A. et al. Genome engineering using the CRISPR-Cas9 system. Nat Protoc 8, 2281-2308 (2013). doi.org:10.1038/nprot.2013.143
71. Zhang, S. et al. Genetically Defined, Syngeneic Organoid Platform for Developing Combination Therapies for Ovarian Cancer. Cancer Discov 11, 362-383 (2021). doi.org:10.1158/2159-8290.CD-20-0455
72. Gibson, D. G. et al. Enzymatic assembly of DNA molecules up to several hundred kilobases. Nat Methods 6, 343-345 (2009). doi.org:10.1038/nmeth.1318

TABLE 6
Amino acid sequences of RNF213 and listed domains of RNF213

| SEQ ID | |
|---|---|
| NO | Sequence (amino acid) |
| SEQ ID | MECPSCQHVSKEETPKFCSQCGERLPPAAPIADSENNNSTMASASEGEME |
| NO: 1 | CGQELKEEGGPCLFPGSDSWQENPEEPCSKASWTVQESKKKKRKKKKKG |
| [RNF213] | NKSASSELASLPLSPASPCHLTLLSNPWPQDTALPHSQAQQSGPTGQPSQPP |
| | GTATTPLEGDGLSAPTEVGDSPLQAQALGEAGVATGSEAQSSPQFQDHTE |
| | GEDQDASIPSGGRGLSQEGTGPPTSAGEGHSRTEDAAQELLLPESKGGSSE |
| | PGTELQTTEQQAGASASMAVDAVAEPANAVKGAGKEMKEKTQRMKQPP |
| | ATTPPFKTHCQEAETKTKDEMAAAEEKVGKNEQGEPEDLKKPEGKNRSA |
| | AAVKNEKEQKNQEADVQEVKASTLSPGGGVTVFFHAIISLHFPFNPDLHK |
| | VFIRGGEEFGESKWDSNICELHYTRDLGHDRVLVEGIVCISKKHLDKYIPY |
| | KYVIYNGESFEYEFIYKHQQKKGEYVNRCLFIKSSLLGSGDWHQYYDIVY |
| | MKPHGRLQKVMNHITDGPRKDLVKGKQIAAALMLDSTFSILQTWDTINLN |
| | SFFTQFEQFCFVLQQPMIYEGQAQLWTDLQYREKEVKRYLWQHLKKHVV |
| | PLPDGKSTDFLPVDCPVRSKLKTGLIVLFVVEKIELLLEGSLDWLCHLLTSD |
| | ASSPDEFHRDLSHILGIPQSWRLYLVNLCQRCMDTRTYTWLGALPVLHCC |
| | MELAPRHKDAWRQPEDTWAALEGLSFSPFREQMLDTSSLLQFMREKQHL |
| | LSIDEPLFRSWFSLLPLSHLVMYMENFIEHLGRFPAHILDCLSGIYYRLPGL |
| | EQVLNTQDVQDVQNVQNILEMLLRLLDTYRDKIPEEALSPSYLTVCLKLH |
| | EAICSSTKLLKFYELPALSAEIVCRMIRLLSLVDSAGQRDETGNNSVQTVFQ |
| | GTLAATKRWLREVFTKNMLTSSGASFTYVKEIEVWRRLVEIQFPAEHGWK |
| | ESLLGDMEWRLTKEEPLSQITAYCNSCWDTKGLEDSVAKTFEKCIIEAVSS |
| | ACQSQTSILQGFSYSDLRKFGIVLSAVITKSWPRTADNFDDILKHLLTLADV |
| | KHVFRLCGTDEKILANVTEDAKRLIAVADSVLTKVVGDLLSGTILVGQLEL |
| | IIKHKNQFLDIWQLREKSLSPQDEQCAVEEALDWRREELLLLKKEKRCVD |
| | SLLKMCGNVKHLIQVDFGVLAVRHSQDLSSKRLNDTVTVRLSTSSNSQRA |
| | THYHLSSQVQEMAGKIDLLRDSHIFQLFWREAAEPLSEPKEDQEAAELLSE |
| | PEEESERHILELEEVYDYLYQPSYRKFIKLHQDLKSGEVTLAEIDVIFKDFV |
| | NKYTDLDSELKIMCTVDHQDQRDWIKDRVEQIKEYHHLHQAVHAAKVIL |
| | QVKESLGLNGDFSVLNTLLNFTDNFDDFRRETLDQINQELIQAKKLLQDIS |
| | EARCKGLQALSLRKEFICWVREALGGINELKVFVDLASISAGENDIDVDRV |
| | ACFHDAVQGYASLLFKLDPSVDFSAFMKHLKKLWKALDKDQYLPRKLCD |
| | SARNLEWLKTVNESHGSVERSSLTLATAINQRGIYVIQAPKGGQKISPDTV |
| | LHLILPESPGSHEESREYSLEEVKELLNKLMLMSGKKDRNNTEVERFSEVF |
| | CSVQRLSQAFIDLHSAGNMLFRTWIAMAYCSPKQGVSLQMDFGLDLVTEL |
| | KEGGDVTELLAALCRQMEHFLDSWKRFVTQKRMEHFYLNFYTAEQLVYL |
| | STELRKQPPSDAALTMLSFIKSNCTLRDVLRASVGCGSEAARYRMRRVME |
| | ELPLMLLSEFSLVDKLRIIMEQSMRCLPAFLPDCLDLETLGHCLAHLAGMG |
| | GSPVERCLPRGLQVGQPNLVVCGHSEVLPAALAVYMQTPSQPLPTYDEVL |
| | LCTPATTFEEVALLLRRCLTLGSLGHKVYSLLFADQLSYEVARQAEELFHN |
| | LCTQQHREDYQLVMVCDGDWEHCYLPSAFSQHKVFVTPQAPLEAIQAYL |
| | AGHYRVPKQTLSAAAVFNDRLCVGIVASERAGVGKSLYVKRLHDKMKM |
| | QLNVKNVPLKTIRLIDPQVDESRVLGALLPFLDAQYQKVPVLFHLDVTSSV |
| | QTGIWVFLFKLLILQYLMDINGKMWLRNPCHLYIVEILERRTSVPSRSSSAL |
| | RTRVPQFSFLDIFPKVTCRPPKEVIDMELSALRSDTEPGMDLWEFCSETFQR |
| | PYQYLRRFNQNQDLDTFQYQEGSVEGTPEECLQHFLFHCGVINPSWSELR |
| | NFARFLNYQLRDCEASLFCNPSFIGDTLRGFKKFVVTFMIFMARDFATPSL |
| | HTSDQSPGKHMVTMDGVREEDLAPFSLRKRWESEPHPYVFFNDDHTTMT |
| | FIGFHLQPNINGSVDAISHLTGKVIKRDVMTRDLYQGLLLQRVPFNVDFDK |
| | LPRHKKLERLCLTLGIPQATDPDKTYELTTDNMLKILAIEMRFRCGIPVIIM |
| | GETGCGKTRLIKFLSDLRRGGTNADTIKLVKVHGGTTADMIYSRVREAEN |
| | VAFANKDQHQLDTILFFDEANTTEAISCIKEVLCDHMVDGQPLAEDSGLHI |
| | IAACNPYRKHSEEMICRLESAGLGYRVSMEETADRLGSIPLRQLVYRVHAL |
| | PPSLIPLVWDFGQLSDVAEKLYIQQIVQRLVESISLDENGTRVITEVLCASQ |
| | GFMRKTEDECSFVSLRDVERCVKVFRWFHEHSAMLLAQLNAFLSKSSVS |
| | KNHTERDPVLWSLMLAIGVCYHASLEKKDSYRKAIARFFPKPYDDSRLLL |
| | DEITRAQDLFLDGVPLRKTIAKNLALKENVFMMVVCIELKIPLFLVGKPGS |
| | SKSLAKTIVADAMQGPAAYSDLFRSLKQVHLVSFQCSPHSTPQGIISTFRQ |
| | CARFQQGKDLQQYVSVVVLDEVGLAEDSPKMPLKTLHPLLEDGCIEDDPA |
| | PHKKVGFVGISNWALDPAKMNRGIFVSRGSPNETELIESAKGICSSDILVQD |
| | RVQGYFASFAKAYETVCKRQDKEFFGLRDYYSLIKMVFAAAKASNRKPS |
| | PQDIAQAVLRNFSGKDDIQALDIFLANLPEAKCSEEVSPMQLIKQNIFGPSQ |
| | KVPGGEQEDAESRYLLVLTKNYVALQILQQTFFEGDQQPEIIFGSGFPKDQ |
| | EYTQLCRNINRVKICMETGKMVLLLNLQNLYESLYDALNQYYVHLGGQK |
| | YVDLGLGTHRVKCRVHPNFRLIVIEEKDVVYKHFPIPLINRLEKHYLDINT |
| | VLEKWQKSIVEELCAWVEKFINVKAHHFQKRHKYSPSDVFIGYHSDACAS |
| | VVLQVIERQGPRALTEELHQKVSEEAKSILLNCATPDAVVRLSAYSLGGFA |
| | AEWLSQEYFHRQRHNSFADFLQAHLHTADLERHAIFTEITTFSRLLTSHDC |
| | EILESEVTGRAPKPTLLWLQQFDTEYSFLKEVRNCLTNTAKCKILIFQTDFE |
| | DGIRSAQLIASAKYSVINEINKIRENEDRIFVYFITKLSRVGRGTAYVGFHG |
| | GLWQSVHIDDLRRSTLMVSDVTRLQHVTISQLFAPGDLPELGLEHRAEDG |
| | HEEAMETEASTSGEVAEVAEEAMETESSEKVGKETSELGGSDVSILDTTRL |
| | LRSCVQSAVGMLRDQNESCTRNMRRVVLLLGLLNEDDACHASFLRVSKM |
| | RLSVFLKKQEESQFHPLEWLAREACNQDALQEAGTFRHTLWKRVQGAVT |
| | PLLASMISFIDRDGNLELLTRPDTPPWARDLWMFIFSDTMLLNIPLVMNNE |
| | RHKGEMAYIVVQNHMNLSENASNNVPFSWKIKDYLEELWVQAQYITDAE |
| | GLPKKFVDIFQQTPLGRFLAQLHGEPQQELLQCYLKDFILLTMRVSTEEEL |
| | KFLQMALWSCTRKLKAASEAPEEEVSLPWVHLAYQRFRSRLQNFSRILTIY |
| | PQVLHSLMEARWNHELAGCEMTLDAFAAMACTEMLTRNTLKPSPQAWL |
| | QLVKNLSMPLELICSDEHMQGSGSLAQAVIREVRAQWSRIFSTALFVEHVL |
| | LGTESRVPELQGLVTEHVFLLDKCLRENSDVKTHGPFEAVMRTLCECKET |
| | ASKTLSRFGIQPCSICLGDAKDPVCLPCDHVHCLRCLRAWFASEQMICPYC |
| | LTALPDEFSPAVSQAHREAIEKHARFRQMCNSFFVDLVSTICFKDNAPPEK |
| | EVIESLLSLLFVQKGRLRDAAQRHCEHTKSLSPFNDVVDKTPVIRSVILKLL |
| | LKYSFHDVKDYIQEYLTLLKKKAFITEDKTELYMLFINCLEDSILEKTSAYS |
| | RNDELNHLEEEGRFLKAYSPASRGREPANEASVEYLQEVARIRLCLDRAA |
| | DFLSEPEGGPEMAKEKQCYLQQVKQFCIRVENDWHRVYLVRKLSSQRGM |
| | EFVQGLSKPGRPHQWVFPKDVVKQQGLRQDHPGQMDRYLVYGDEYKAL |
| | RDAVAKAVLECKPLGIKTALKACKTPQSQQSAYFLLTLFREVAILYRSHN |
| | ASLHPTPEQCEAVSKFIGECKILSPPDISRFATSLVDNSVPLLRAGPSDSNLD |
| | GTVTEMAIHAAAVLLCGQNELLEPLKNLAFSPATMAHAFLPTMPEDLLAQ |
| | ARRWKGLERVHWYTCPNGHPCSVGECGRPMEQSICIDCHAPIGGIDHKPR |
| | DGFHLVKDKADRTQTGHVLGNPQRRDVVTCDRGLPPVVFLLIRLLTHLAL |
| | LLGASQSSQALINIIKPPVRDPKGFLQQHILKDLEQLAKMLGHSADETIGVV |
| | HLVLRRLLQEQHQLSSRRLLNFDTELSTKEMRNNWEKEIAAVISPELEHLD |
| | KTLPTMNNLISQDKRISSNPVAKIIYGDPVTFLPHLPRKSVVHCSKIWSCRK |
| | RITVEYLQHIVEQKNGKERVPILWHFLQKEAELRLVKFLPEILALQRDLVK |
| | QFQNVQQVEYSSIRGFLSKHSSDGLRQLLHNRITVFLSTWNKLRRSLETNG |
| | EINLPKDYCSTDLDLDTEFEILLPRRRGLGLCATALVSYLIRLHNEIVYAVE |
| | KLSKENNSYSVDAAEVTELHVISYEVERDLTPLILSNCQYQVEEGRETVQE |
| | FDLEKIQRQIVSRFLQGKPRLSLKGIPTLVYRHDWNYEHLFMDIKNKMAQ |
| | DSLPSSVISAISGQLQSYSDACEVLSVVEVTLGFLSTAGGDPNMQLNVYTQ |
| | DILQMGDQTIHVLKALNRCQLKHTIALWQFLSAHKSEQLLRLHKEPFGEIS |
| | SRYKADLSPENAKLLSTFLNQTGLDAFLLELHEMIILKLKNPQTQTEERFRP |
| | QWSLRDTLVSYMQTKESEILPEMASQFPEEILLASCVSVWKTAAVLKWNR |
| | EMR* |
| SEQ ID | EYVNRCLFIKSSLLGSGDWHQYYDIVYMKPHGRLQKVMNHITDGPRKDL |
| NO: 2 | VKGKQIAAALMLDSTFSILQTWDTINLNSFFTQFEQFCFVLQQPMIYEGQA |
| [N-Arm | QLWTDLQYREKEVKRYLWQHLKKHVVPLPDGKSTDFLPVDCPVRSKLKT |
| domain] | GLIVLFVVEKIELLLEGSLDWLCHLLTSDASSPDEFHRDLSHILGIPQSWRL |
| | YLVNLCQRCMDTRTYTWLGALPVLHCCMELAPRHKDAWRQPEDTWAA |
| | LEGLSFSPFREQMLDTSSLLQFMREKQHLLSIDEPLFRSWFSLLPLSHLVMY |
| | MENFIEHLGRFPAHILDCLSGIYYRLPGLEQVLNTQDVQDVQNVQNILEML |
| | LRLLDTYRDKIPEEALSPSYLTVCLKLHEAICSSTKLLKFYELPALSAEIVCR |
| | MIRLLSLVDSAGQRDETGNNSVQTVFQGTLAATKRWLREVFTKNMLTSS |
| | GASFTYVKEIEVWRRLVEIQFPAEHGWKESLLGDMEWRLTKEEPLSQITA |
| | YCNSCWDTKGLEDSVAKTFEKCIIEAVSSACQSQTSILQGFSYSDLRKFGIV |
| | LSAVITKSWPRTADNFDDILKHLLTLADVKHVFRLCGTDEKILANVTEDA |
| | KRLIAVADSVLTKVVGDLLSGTILVGQLELIIKHKNQFLDIWQLREKSLSPQ |
| | DEQCAVEEALDWRREELLLLKKEKRCVDSLLKMCGNVKHLIQVDFGVLA |
| | VRHSQDLSSKRLNDTVTVRLSTSSNSQRATHYHLSSQVQEMAGKIDLLRD |
| | SHIFQLFWREAAEPLSEPKEDQEAAELLSEPEEESERHILELEEVYDYLYQP |
| | SYRKFIKLHQDLKSGEVTLAEIDVIFKDFVNKYTDLDSELKIMCTVD |
| SEQ ID | HQDQRDWIKDRVEQIKEYHHLHQAVHAAKVILQVKESLGLNGDFSVLNT |
| NO: 3 | LLNFTDNFDDFRRETLDQINQELIQAKKLLQDISEARCKGLQALSLRKEFIC |
| [Linker | WVREALGGINELKVFVDLASISAGENDIDVDRVACFHDAVQGYASLLFKL |
| domain] | DPSVDFSAFMKHLKKLWKALDKDQYLPRKLCDSARNLEWLKTVNESHGS |
| | VERSSLTLATAINQRGIYVIQAPKGGQKISPDTVLHLILPESPGSHEESREYS |
| | LEEVKELLNKLMLMSGKKDRNNTEVERFSEVFCSVQRLSQAFIDLHSAGN |
| | MLFRTWIAMAYCSPKQGVSLQMDFGLDLVTELKEGGDVTELLAALCRQ |
| | MEHFLDSWKRFVTQKRMEHFYLNFYTAEQLVYLSTELRKQPPSDAALTM |
| | LSFIKSNCTLRDVLRASVGCGSEAARYRMRRVMEELPLMLLSEFSLVDKL |
| | RIIMEQSMRCLPAFLPD |
| SEQ ID | CLDLETLGHCLAHLAGMGGSPVERCLPRGLQVGQPNLVVCGHSEVLPAA |
| NO: 4 | LAVYMQTPSQPLPTYDEVLLCTPATTFEEVALLLRRCLTLGSLGHKVYSLL |
| [AAA | FADQLSYEVARQAEELFHNLCTQQHREDYQLVMVCDGDWEHCYLPSAFS |
| ATPASE | QHKVFVTPQAPLEAIQAYLAGHYRVPKQTLSAAAVFNDRLCVGIVASERA |
| domain] | GVGKSLYVKRLHDKMKMQLNVKNVPLKTIRLIDPQVDESRVLGALLPFL |
| | DAQYQKVPVLFHLDVTSSVQTGIWVFLFKLLILQYLMDINGKMWLRNPC |
| | HLYIVEILERRTSVPSRSSSALRTRVPQFSFLDIFPKVTCRPPKEVIDMELSA |
| | LRSDTEPGMDLWEFCSETFQRPYQYLRRFNQNQDLDTFQYQEGSVEGTPE |
| | ECLQHFLFHCGVINPSWSELRNFARFLNYQLRDCEASLFCNPSFIGDTLRGF |
| | KKFVVTFMIFMARDFATPSLHTSDQSPGKHMVTMDGVREEDLAPFSLRKR |
| | WESEPHPYVFFNDDHTTMTFIGFHLQPNINGSVDAISHLTGKVIKRDVMTR |
| | DLYQGLLLQRVPFNVDFDKLPRHKKLERLCLTLGIPQATDPDKTYELTTD |
| | NMLKILAIEMRFRCGIPVIIMGETGCGKTRLIKFLSDLRRGGTNADTIKLVK |
| | VHGGTTADMIYSRVREAENVAFANKDQHQLDTILFFDEANTTEAISCIKEV |
| | LCDHMVDGQPLAEDSGLHIIAACNPYRKHSEEMICRLESAGLGYRVSMEE |
| | TADRLGSIPLRQLVYRVHALPPSLIPLVWDFGQLSDVAEKLYIQQIVQRLV |
| | ESISLDENGTRVITEVLCASQGFMRKTEDECSFVSLRDVERCVKVFRWFHE |
| | HSAMLLAQLNAFLSKSSVSKNHTERDPVLWSLMLAIGVCYHASLEKKDS |
| | YRKAIARFFPKPYDDSRLLLDEITRAQDLFLDGVPLRKTIAKNLALKENVF |
| | MMVVCIELKIPLFLVGKPGSSKSLAKTIVADAMQGPAAYSDLFRSLKQVH |
| | LVSFQCSPHSTPQGIISTFRQCARFQQGKDLQQYVSVVVLDEVGLAEDSPK |
| | MPLKTLHPLLEDGCIEDDPAPHKKVGFVGISNWALDPAKMNRGIFVSRGS |
| | PNETELIESAKGICSSDILVQDRVQGYFASFAKAYETVCKRQDKEFFGLRD |
| | YYSLIKMVFAAAKASNRKPSPQDIAQAVLRNFSGKDDIQALDIFLANLPEA |
| | KCSEEVSPMQLIKQNIFGPSQKVPGGEQEDAESRYLLVLTKNYVALQILQQ |
| | TFFEGDQQPEIIFGSGFPKDQEYTQLCRNINRVKICMETGKMVLLLNLQNL |
| | YESLYDALNQYYVHLGGQKYVDLGLGTHRVKCRVHPNFRLIVIEEKDVV |
| | YKHFPIPLINRLEKHYLDINTVLEKWQKSIVEELCAWVEKFINVKAHHFQK |
| | RHKYSPSDVFIGYHSDACASVVLQVIERQGPRALTEELHQKVSEEAKSILL |
| | NCATPDAVVRLSAYSLGGFAAEWLSQEYFHRQRHNSFADFLQAHLHTAD |
| | LERHAIFTEITTFSRLLTSHDCEILESEVTGRAPKPTLLWLQQFDTEYSFLKE |
| | VRNCLTNTAKCKILIFQTDFEDGIRSAQLIASAKYSVINEINKIRENEDRIFV |
| | YFITKLSRVGRGTAYVGFHGGLWQSVHIDDLRR |
| SEQ ID | CLDLETLGHCLAHLAGMGGSPVERCLPRGLQVGQPNLVVCGHSEVLPAA |
| NO: 5 | LAVYMQTPSQPLPTYDEVLLCTPATTFEEVALLLRRCLTLGSLGHKVYSLL |
| [AAA 1 | FADQLSYEVARQAEELFHNLCTQQHREDYQLVMVCDGDWEHCYLPSAFS |
| domain] | QHKVFVTPQAPLEAIQAYLAGHY |
| SEQ ID | RVPKQTLSAAAVFNDRLCVGIVASERAGVGKSLYVKRLHDKMKMQLNV |
| NO: 6 | KNVPLKTIRLIDPQVDESRVLGALLPFLDAQYQKVPVLFHLDVTSSVQTGI |
| [AAA 2 | WVFLFKLLILQYLMDINGKMWLRNPCHLYIVEILERRTSVPSRSSSALRTR |
| domain] | VPQFSFLDIFPKVTCRPPKEVIDMELSALRSDTEPGMDLWEFCSETFQRPY |
| | QYLRRFNQNQDLDTFQYQEGSVEGTPEECLQHFLFHCGVINPSWSELRNF |
| | ARFLNYQLRDCEASLFCNPSFIGDTLRGFKKFVVTFMIFMARDFATPSL |
| SEQ ID | TTDNMLKILAIEMRFRCGIPVIIMGETGCGKTRLIKFLSDLRRGGTNADTIK |
| NO: 7 | LVKVHGGTTADMIYSRVREAENVAFANKDQHQLDTILFFDEANTTEAISCI |
| [AAA 3 | KEVLCDHMVDGQPLAEDSGLHIIAACNPYRKHSEEMICRLESAGLGYRVS |
| domain] | MEETADRLGSIPLRQLVYRVHALPPSLIPLVWDFGQLSDVAEKLYIQQIVQ |
| | RLVESISLDENGTRVITEVLCASQGFMRKTEDECSFVSLRDVERCVKVFRW |
| | FHEHSAMLLAQLNAFLSKSSVSKNHTERDPVLWSLMLAIGVCYHASLEKK |
| | DSYRKAIARFFPKPYDDSRLLLDEITRAQDLFLDGVPLRKTIAK |
| SEQ ID | NLALKENVFMMVVCIELKIPLFLVGKPGSSKSLAKTIVADAMQGPAAYSD |
| NO: 8 | LFRSLKQVHLVSFQCSPHSTPQGIISTFRQCARFQQGKDLQQYVSVVVLDE |
| [AAA 4 | VGLAEDSPKMPLKTLHPLLEDGCIEDDPAPHKKVGFVGISNWALDPAKMN |
| domain] | RGIFVSRGSPNETELIESAKGICSSDILVQDRVQGYFASFAKAYETVCKRQD |
| | KEFFGLRDYYSLIKMVFAAAKASNRKPSPQDIAQAVLRNFSGKDDIQALDI |
| | FLANLPEAKCSEE |
| SEQ ID | VSPMQLIKQNIFGPSQKVPGGEQEDAESRYLLVLTKNYVALQILQQTFFEG |
| NO: 9 | DQQPEIIFGSGFPKDQEYTQLCRNINRVKICMETGKMVLLLNLQNLYESLY |
| [AAA 5 | DALNQYYVHLGGQKYVDLGLGTHRVKCRVHPNFRLIVIEEKDVVYKHFPI |
| domain] | PLINRLEKHYLDINTVLEKWQKSIVEELCAWVEKFINVKAHHFQKRHKYS |
| | PSDVFIGYHSDACASVVLQVIERQGPRALTEELHQKVSEEAKSILLNCATP |
| | DAVVRLSAYSLGGFAAEWLSQEYFHRQR |
| SEQ ID | HNSFADFLQAHLHTADLERHAIFTEITTFSRLLTSHDCEILESEVTGRAPKPT |
| NO: 10 | LLWLQQFDTEYSFLKEVRNCLTNTAKCKILIFQTDFEDGIRSAQLIASAKYS |
| [AAA 6 | VINEINKIRENEDRIFVYFITKLSRVGRGTAYVGFHGGLWQSVHIDDLRR |
| domain] | |
| SEQ ID | STLMVSDVTRLQHVTISQLFAPGDLPELGLEHRAEDGHEEAMETEASTSGE |
| NO: 11 | VAEVAEEAMETESSEKVGKETSELGGSDVSILDTTRLLRSCVQSAVGMLR |
| [Hinge | DQNESCTRNMRRVVLLLGLLNEDDACHASFLRVSKMRLSVFLKKQEESQ |
| domain] | FHPLEWLAREACNQDALQEAGTFRHTLWKRVQGAVTPLLASMISFI |
| SEQ ID | DRDGNLELLTRPDTPPWARDLWMFIFSDTMLLNIPLVMNNERHKGEMAYI |
| NO: 12 | VVQNHMNLSENASNNVPFSWKIKDYLEELWVQAQYITDAEGLPKKFVDIF |
| [E3 | QQTPLGRFLAQLHGEPQQELLQCYLKDFILLTMRVSTEEELKFLQMALWS |
| Ligase | CTRKLKAASEAPEEEVSLPWVHLAYQRFRSRLQNFSRILTIYPQVLHSLME |
| domain] | ARWNHELAGCEMTLDAFAAMACTEMLTRNTLKPSPQAWLQLVKNLSMP |
| | LELICSDEHMQGSGSLAQAVIREVRAQWSRIFSTALFVEHVLLGTESRVPE |
| | LQGLVTEHVFLLDKCLRENSDVKTHGPFEAVMRTLCECKETASKTLSRFGI |
| | QPCSICLGDAKDPVCLPCDHVHCLRCLRAWFASEQMICPYCLTALPDEFSP |
| | AVSQAHREAIEKHARFRQMCNSFFVDLVSTICFKDNAPPEKEVIESLLSLLF |
| | VQKGRLRDAAQRHCEHTKSLSPFNDVVDKTPVIRSVILKLLLKYSFHDVK |
| | DYIQEYLTLLKKKAFITEDKTELYMLFINCLEDSILEKTSAYSRNDELNHLE |
| | EEGRFLKAYSPASRGREPANEASVEYLQEVARIRLCLDRAADFLSEPEGGP |
| | EMAKEKQCYLQQVKQFCIRVENDWHRVYLVRKLSSQRGMEFVQGLSKP |
| | GRPHQWVFPKDVVKQQGLRQDHPGQMDRYLVYGDEYKALRDAVAKAV |
| | LECKPLGIKTALKACKTPQSQQSAYFLLTLFREVAILYRSHNASLHPTPEQC |
| | EAVSKFIGECKILSPPDISRFATSLVDNSVPLLRAGPSDSNLDGTVTEMAIH |
| | AAAVLLCGQNELLEPLKNLAFSPATMAHAFLPTMPEDLLAQARRWKGLE |
| | RVHWYTCPNGHPCSVGECGRPMEQSICIDCHAPIGGIDHKPRDGFHLVKD |
| | KADRTQTGHVLGNPQRRDVVTCDRGLPPVVFLLIRLLTHLALLLGASQSS |
| | QALINIIKPPVRDPKGFLQQHILKDLEQLAKMLGHSADETIGVVHLVLRRL |
| | LQEQHQLSSRRLLNFDTELSTKEMRNNWEKEIAAVISPELEHLDKTLPTMN |
| | NLISQDKRISSNPVAKIIYGDPVTFLPHLPRKSVVHCSKIWSCRKRITVEYL |
| | QHIVEQKNGKERVPILWHFLQKEAELRLVKFLPEILALQRDLVKQFQNVQ |
| | QVEYSSIRGFLSKHSSDGLRQLLHNRITVFLSTWNKLRRSLETNGEINLPKD |
| | YCSTDLDLDTEFEILLPRRRGLGLCATALVSYLIRLHNEIVYAVEKLSKENN |
| | SYSVDAAEVTELHVISYEVERDLTPLILSNCQYQVEEGRETVQEFDLEKIQ |
| | RQIVSRFLQGKPRLSLKGIPTLVYRHD |
| SEQ ID | DRDGNLELLTRPDTPPWARDLWMFIFSDTMLLNIPLVMNNERHKGEMAYI |
| NO: 13 | VVQNHMNLSENASNNVPFSWKIKDYLEELWVQAQYITDAEGLPKKFVDIF |
| [BACK | QQTPLGRFLAQLHGEPQQELLQCYLKDFILLTMRVSTEEELKFLQMALWS |
| domain] | CTRKLKAASEAPEEEVSLPWVHLAYQRFRSRLQNFSRILTIYPQVLHSLME |
| | ARWNHELAGCEMTLDAFAAMACTEMLTRNTLKPSPQAWLQLVKNLSMP |
| | LELICSDEHMQGSGSLAQAVIREVRAQWSRIFSTALFVEHVLLGTESRVPE |
| | LQGLVTEHVFLLDKCLRENSDVKTHGPFEAVMRTLCECKETASKTL |
| SEQ ID | SRFGIQPCSICLGDAKDPVCLPCDHVHCLRCLRAWFASEQMICPYCLTALP |
| NO: 14 | DEFSPAVSQA |
| [RING | |
| domain] | |
| SEQ ID | HREAIEKHARFRQMCNSFFVDLVSTICFKDNAPPEKEVIESLLSLLFVQKGR |
| NO: 15 | LRDAAQRHCEHTKSLSPFNDVVDKTPVIRSVILKLLLKYSFHDVKDYIQEY |
| [SHELL | LTLLKKKAFITEDKTELYMLFINCLEDSILEKTSAYSRNDELNHLEEEGRFL |
| domain] | KAYSPASRGREPANEASVEYLQEVARIRLCLDRAADFLSEPEGGPEMAKE |
| | KQCYLQQVKQFCIRVENDWHRVYLVRKLSSQRGMEFVQGLSKPGRPHQ |
| | WVFPKDVVKQQGLRQDHPGQMDRYLVYGDEYKALRDAVAKAVLECKP |
| | LGIKTALKACKTPQSQQSAYFLLTLFREVAILYRSHNASLHPTPEQCEAVS |
| | KFIGECKILSPPDISRFATSLVDNSVPLLRAGPSDSNLDGTVTEMAIHAAAV |
| | LLCGQNELLEPLKNLAFSPATMAHAFLPTMPEDLLAQARRWKGLERVHW |
| | YTCPNGHPCSVGECGRPMEQSICIDCHAPIGGIDHKPRDGFHLVKDKADRT |
| | QTGHVLGNPQRRDVVTCDRGLPPVVFLLIRLLTHLALLLGASQSSQALINII |
| | KPPVRDPKGFLQQHILKDLEQLAKMLGHSADETIGVVHLVLRRLLQEQHQ |
| | LSSRRLLNFDTELSTKEMRNNWEKEIAAVISPELEHLDKTLPTMNNLISQD |
| | KRISSNPVAKIIYGDPVTFLPHLPRKSVVHCSKIWSCRKR |
| SEQ ID | MPEDLLAQARRWKGLERVHWYTCPNGHPCSVGECGRPMEQSICIDCHAPI |
| NO: 4589 | GGIDHKPRDGFHLVKDKADRTQT |
| [RZ | |
| domain] | |
| SEQ ID | ITVEYLQHIVEQKNGKERVPILWHFLQKEAELRLVKFLPEILALQRDLVKQ |
| NO: 16 | FQNVQQVEYSSIRGFLSKHSSDGLRQLLHNRITVFLSTWNKLRRSLETNGEI |
| [CORE | NLPKDYCSTDLDLDTEFEILLPRRRGLGLCATALVSYLIRLHNEIVYAVEKL |
| domain] | SKENNSYSVDAAEVTELHVISYEVERDLTPLILSNCQYQVEEGRETVQEFD |
| | LEKIQRQIVSRFLQGKPRLSLKGIPTLVYRHD |
| SEQ ID | WNYEHLFMDIKNKMAQDSLPSSVISAISGQLQSYSDACEVLSVVEVTLGFL |
| NO: 17 | STAGGDPNMQLNVYTQDILQMGDQTIHVLKALNRCQLKHTIALWQFLSA |
| [CTD | HKSEQLLRLHKEPFGEISSRYKADLSPENAKLLSTFLNQTGLDAFLLELHE |
| domain] | MIILKLKNPQTQTEERFRPQWSLRDTLVSYMQTKESEILPEMASQFPEEILL |
| | ASCVSVWKTAAVLKWNREMR |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

TABLE 7
RNF213 Peptides Rep 1

| | Peptide | | | | | | | | Start- | | | | |
|---|---|---|---|---|---|---|---|---|---|---|---|---|---|
| SEQ | < Protein- | Modifi- | | | | Calc. | Off- | Mass | ing | | | | |
| ID | Metrics | cation | Observed | | Observed | mass | by-x | error | posi- | | | Delta | Log |
| NO: | Confidential > | Type(s) | m/z | z | (M+H) | (M+H) | error | (ppm) | tion | Score | Delta | Mod | Prob |
| 880 | K.ASWTVQESK.K | | 518.259 | 2 | 1035.510 | 1035.511 | 0 | −0.1 | 81 | 500.2 | 500.2 | 500.2 | 7.57 |
| 884 | R.GLSQEGTGPPTSA | | 608.954 | 3 | 1824.849 | 1824.847 | 0 | 0.9 | 215 | 822.9 | 822.9 | 822.9 | 14.95 |
| | GEGHSR.T | | | | | | | | | | | | |
| 884 | R.GLSQEGTGPPTSA | | 608.954 | 3 | 1824.849 | 1824.847 | 0 | 0.9 | 215 | 818.4 | 818.4 | 818.4 | 14.95 |
| | GEGHSR.T | | | | | | | | | | | | |
| 1079 | R.GLSQEGTGPPTSA | | 1117.209 | 3 | 3349.614 | 3349.614 | 0 | −0.2 | 215 | 808.3 | 808.3 | 808.3 | 14.39 |
| | GEGHSRTEDAAQELL | | | | | | | | | | | | |
| | LPESK.G | | | | | | | | | | | | |
| 884 | R.GLSQEGTGPPTSA | | 608.955 | 3 | 1824.850 | 1824.847 | 0 | 1.4 | 215 | 688.2 | 688.2 | 688.2 | 11.96 |
| | GEGHSR.T | | | | | | | | | | | | |
| 884 | R.GLSQEGTGPPTSA | | 608.954 | 3 | 1824.847 | 1824.847 | 0 | 0.0 | 215 | 652.0 | 652.0 | 652.0 | 11.96 |
| | GEGHSR.T | | | | | | | | | | | | |
| 1079 | R.GLSQEGTGPPTSA | | 1117.209 | 3 | 3349.613 | 3349.614 | 0 | −0.3 | 215 | 626.6 | 626.6 | 626.6 | 10.39 |
| | GEGHSRTEDAAQELL | | | | | | | | | | | | |
| | LPESK.G | | | | | | | | | | | | |
| 884 | R.GLSQEGTGPPTSA | | 912.927 | 2 | 1824.847 | 1824.847 | 0 | −0.1 | 215 | 618.5 | 618.5 | 618.5 | 10.96 |
| | GEGHSR.T | | | | | | | | | | | | |
| 1079 | R.GLSQEGTGPPTSA | | 1117.210 | 3 | 3349.616 | 3349.614 | 0 | 0.5 | 215 | 606.0 | 606.0 | 606.0 | 10.39 |
| | GEGHSRTEDAAQELL | | | | | | | | | | | | |
| | LPESK.G | | | | | | | | | | | | |
| 884 | R.GLSQEGTGPPTSA | | 456.967 | 4 | 1824.847 | 1824.847 | 0 | 0.1 | 215 | 588.5 | 588.5 | 588.5 | 9.37 |
| | GEGHSR.T | | | | | | | | | | | | |
| 884 | R.GLSQEGTGPPTSA | | 608.954 | 3 | 1824.848 | 1824.847 | 0 | 0.7 | 215 | 573.3 | 573.3 | 573.3 | 9.96 |
| | GEGHSR.T | | | | | | | | | | | | |
| 842 | R.GLSQEGTGPPTSA | | 1358.849 | 5 | 6790.214 | 6790.214 | 0 | 0.0 | 215 | 570.6 | 570.6 | 570.6 | 6.65 |
| | GEGHSRTEDAAQELL | | | | | | | | | | | | |
| | LPESKGGSSEPGTEL | | | | | | | | | | | | |
| | QTTEQQAGASASMAV | | | | | | | | | | | | |
| | DAVAEPANAVK.G | | | | | | | | | | | | |
| 1079 | R.GLSQEGTGPPTSA | | 838.159 | 4 | 3349.614 | 3349.614 | 0 | 0.2 | 215 | 543.1 | 543.1 | 543.1 | 18.55 |
| | GEGHSRTEDAAQELL | | | | | | | | | | | | |
| | LPESK.G | | | | | | | | | | | | |
| 1079 | R.GLSQEGTGPPTSA | | 1117.543 | 3 | 3350.616 | 3349.614 | 1 | −0.6 | 215 | 494.1 | 494.1 | 494.1 | 7.63 |
| | GEGHSRTEDAAQELL | | | | | | | | | | | | |
| | LPESK.G | | | | | | | | | | | | |
| 884 | R.GLSQEGTGPPTSA | | 608.954 | 3 | 1824.848 | 1824.847 | 0 | 0.7 | 215 | 426.1 | 426.1 | 426.1 | 7.83 |
| | GEGHSR.T | | | | | | | | | | | | |
| 1079 | R.GLSQEGTGPPTSA | | 670.729 | 5 | 3349.616 | 3349.614 | 0 | 0.5 | 215 | 399.4 | 399.4 | 399.4 | 5.91 |
| | GEGHSRTEDAAQELL | | | | | | | | | | | | |
| | LPESK.G | | | | | | | | | | | | |
| 1079 | R.GLSQEGTGPPTSA | | 838.159 | 4 | 3349.613 | 3349.614 | 0 | −0.4 | 215 | 386.7 | 386.7 | 386.7 | 6.19 |
| | GEGHSRTEDAAQELL | | | | | | | | | | | | |
| | LPESK.G | | | | | | | | | | | | |
| 884 | R.GLSQEGTGPPTSA | | 912.927 | 2 | 1824.848 | 1824.847 | 0 | 0.3 | 215 | 386.5 | 386.5 | 386.5 | 8.45 |
| | GEGHSR.T | | | | | | | | | | | | |
| 884 | R.GLSQEGTGPPTSA | | 608.954 | 3 | 1824.849 | 1824.847 | 0 | 0.9 | 215 | 369.0 | 369.0 | 369.0 | 7.47 |
| | GEGHSR.T | | | | | | | | | | | | |
| 1185 | R.TEDAAQELLLPES | | 772.397 | 2 | 1543.786 | 1543.785 | 0 | 0.5 | 234 | 677.9 | 603.5 | 603.5 | 11.96 |
| | K.G | | | | | | | | | | | | |
| 1185 | R.TEDAAQELLLPES | | 772.396 | 2 | 1543.785 | 1543.785 | 0 | 0.1 | 234 | 621.1 | 580.2 | 580.2 | 10.96 |
| | K.G | | | | | | | | | | | | |
| 1078 | K.GGSSEPGTELQTT | | 943.953 | 4 | 3772.791 | 3772.793 | 0 | −0.4 | 248 | 967.2 | 967.2 | 967.2 | 15.65 |
| | EQQAGASASMAVDAV | | | | | | | | | | | | |
| | AEPANAVKGAGK.E | | | | | | | | | | | | |
| 1210 | K.GGSSEPGTELQTT | | 1040.999 | 4 | 4160.973 | 4160.971 | 0 | 0.5 | 248 | 933.0 | 933.0 | 933.0 | 15.65 |
| | EQQAGASASMAVDAV | | | | | | | | | | | | |
| | AEPANAVKGAGKEMK | | | | | | | | | | | | |
| | .E | | | | | | | | | | | | |
| 1078 | K.GGSSEPGTELQTT | | 1258.268 | 3 | 3772.790 | 3772.793 | 0 | 0.9 | 248 | 918.8 | 918.8 | 918.8 | 15.26 |
| | EQQAGASASMAVDAV | | | | | | | | | | | | |
| | AEPANAVKGAGK.E | | | | | | | | | | | | |
| 1078 | K.GGSSEPGTELQTT | | 1258.270 | 3 | 3772.795 | 3772.793 | 0 | 0.6 | 248 | 914.0 | 914.0 | 914.0 | 15.95 |
| | EQQAGASASMAVDAV | | | | | | | | | | | | |
| | AEPANAVKGAGK.E | | | | | | | | | | | | |
| 4620 | K.GGSSEPGTELQTT | N[+1] | 1258.930 | 3 | 3774.776 | 3773.777 | 1 | −1.2 | 248 | 826.0 | 826.0 | 183.5 | 13.26 |
| | EQQAGASASMAVDAV | | | | | | | | | | | | |
| | AEPAN[+0.984]AV | | | | | | | | | | | | |
| | KGAGK.E | | | | | | | | | | | | |
| 1210 | K.GGSSEPGTELQTT | | 1040.998 | | 4160.972 | 4160.971 | 0 | 0.1 | 248 | 825.8 | 825.8 | 825.8 | 13.66 |
| | EQQAGASASMAVDAV | | | | | | | | | | | | |
| | AEPANAVKGAGKEMK | | | | | | | | | | | | |
| | .E | | | | | | | | | | | | |
| 1567 | K.GGSSEPGTELQTT | M[+16] | 1159.208 | 3 | 3475.611 | 3475.613 | 0 | −0.6 | 248 | 750.7 | 750.7 | 750.7 | 11.86 |
| | EQQAGASASM | | | | | | | | | | | | |
| | [+15.995]AVDA | | | | | | | | | | | | |
| | VAEPANAVK.G | | | | | | | | | | | | |
| 1066 | K.GGSSEPGTELQTT | | 1154.545 | 3 | 3461.622 | 3459.618 | 2 | 0.9 | 248 | 728.4 | 536.0 | 325.1 | 10.86 |
| | EQQAGASASMAVDAV | | | | | | | | | | | | |
| | AEPANAVK.G | | | | | | | | | | | | |
| 1066 | K.GGSSEPGTELQTT | | 1153.877 | 3 | 3459.617 | 3459.618 | 0 | 0.3 | 248 | 726.1 | 726.1 | 726.1 | 11.56 |
| | EQQAGASASMAVDAV | | | | | | | | | | | | |
| | AEPANAVK.G | | | | | | | | | | | | |
| 1567 | K.GGSSEPGTELQTT | M[+16] | 1159.209 | 3 | 3475.614 | 3475.613 | 0 | 0.2 | 248 | 713.1 | 713.1 | 713.1 | 11.56 |
| | EQQAGASASM | | | | | | | | | | | | |
| | [+15.995]AVDAV | | | | | | | | | | | | |
| | AEPANA | | | | | | | | | | | | |
| | VK.G | | | | | | | | | | | | |
| 4621 | K.GGSSEPGTELQTT | M[+16] | 1044.996 | | 4176.963 | 4176.966 | 0 | −0.8 | 248 | 696.1 | 607.7 | 339.8 | 8.98 |
| | EQQAGASASMAVDAV | | | | | | | | | | | | |
| | AEPANAVKGAGKEM | | | | | | | | | | | | |
| | [+15.995]K.E | | | | | | | | | | | | |
| 1066 | K.GGSSEPGTELQTT | | 865.660 | 4 | 3459.619 | 3459.618 | 0 | 0.3 | 248 | 687.8 | 687.8 | 687.8 | 10.56 |
| | EQQAGASASMAVDAV | | | | | | | | | | | | |
| | AEPANAVK.G | | | | | | | | | | | | |
| 1066 | K.GGSSEPGTELQTT | | 1154.211 | 3 | 3460.619 | 3459.618 | 1 | −0.8 | 248 | 687.7 | 687.7 | 368.5 | 9.86 |
| | EQQAGASASMAVDAV | | | | | | | | | | | | |
| | AEPANAVK.G | | | | | | | | | | | | |
| 1066 | K.GGSSEPGTELQTT | | 865.660 | 4 | 3459.619 | 3459.618 | 0 | 0.3 | 248 | 685.4 | 685.4 | 685.4 | 10.56 |
| | EQQAGASASMAVDAV | | | | | | | | | | | | |
| | AEPANAVK.G | | | | | | | | | | | | |
| 4622 | K.GGSSEPGTELQTT | M[+16] | 1045.245 | 4 | 4177.959 | 4176.966 | 1 | 2.5 | 248 | 669.8 | 669.8 | 281.0 | 9.22 |
| | EQQAGASASM | | | | | | | | | | | | |
| | [+15.995] | | | | | | | | | | | | |
| | AVDAVAEPANA | | | | | | | | | | | | |
| | VKGAGKEMK.E | | | | | | | | | | | | |
| 1066 | K.GGSSEPGTELQTT | | 1153.878 | 3 | 3459.620 | 3459.618 | 0 | 0.6 | 248 | 663.5 | 613.2 | 613.2 | 10.56 |
| | EQQAGASASMAVDAV | | | | | | | | | | | | |
| | AEPANAVK.G | | | | | | | | | | | | |
| 1066 | K.GGSSEPGTELQTT | | 865.660 | 4 | 3459.620 | 3459.618 | 0 | 0.5 | 248 | 651.9 | 651.9 | 651.9 | 10.56 |
| | EQQAGASASMAVDAV | | | | | | | | | | | | |
| | AEPANAVK.G | | | | | | | | | | | | |
| 1066 | K.GGSSEPGTELQTT | | 1153.878 | 3 | 3459.621 | 3459.618 | 0 | 0.7 | 248 | 649.4 | 649.4 | 649.4 | 9.56 |
| | EQQAGASASMAVDAV | | | | | | | | | | | | |
| | AEPANAVK.G | | | | | | | | | | | | |
| 1066 | K.GGSSEPGTELQTT | | 1153.878 | 3 | 3459.618 | 3459.618 | 0 | 0.0 | 248 | 616.6 | 616.6 | 616.6 | 8.83 |
| | EQQAGASASMAVDAV | | | | | | | | | | | | |
| | AEPANAVK.G | | | | | | | | | | | | |
| 4621 | K.GGSSEPGTELQTT | M[+16] | 1044.996 | 4 | 4176.963 | 4176.966 | 0 | 0.7 | 248 | 615.2 | 525.6 | 409.4 | 17.75 |
| | EQQAGASASMAVDAV | | | | | | | | | | | | |
| | AEPANAVKGAGKEM | | | | | | | | | | | | |
| | [+15.995]K.E | | | | | | | | | | | | |
| 1066 | K.GGSSEPGTELQTT | | 865.660 | 4 | 3459.618 | 3459.618 | 0 | 0.0 | 248 | 598.2 | 598.2 | 598.2 | 7.83 |
| | EQQAGASASMAVDAV | | | | | | | | | | | | |
| | AEPANAVK.G | | | | | | | | | | | | |
| 1567 | K.GGSSEPGTELQTT | M[+16] | 1159.544 | 3 | 3476.616 | 3475.613 | 1 | 0.0 | 248 | 548.2 | 548.2 | 345.9 | 6.75 |
| | EQQAGASASM | | | | | | | | | | | | |
| | [+15.995] | | | | | | | | | | | | |
| | AVDAVAEPANA | | | | | | | | | | | | |
| | VK.G | | | | | | | | | | | | |
| 1066 | K.GGSSEPGTELQTT | | 1153.881 | 3 | 3459.629 | 3459.618 | 0 | 3.0 | 248 | 546.7 | 492.8 | 492.8 | 5.28 |
| | EQQAGASASMAVDAV | | | | | | | | | | | | |
| | AEPANAVK.G | | | | | | | | | | | | |
| 1078 | K.GGSSEPGTELQTT | | 944.204 | 4 | 3773.794 | 3772.793 | 1 | −0.8 | 248 | 521.3 | 293.3 | 293.3 | 4.19 |
| | EQQAGASASMAVDAV | | | | | | | | | | | | |
| | AEPANAVKGAGK.E | | | | | | | | | | | | |
| 1066 | K.GGSSEPGTELQTT | | 865.667 | 4 | 3459.646 | 3459.618 | 0 | 8.1 | 248 | 309.1 | 309.1 | 309.1 | 2.61 |
| | EQQAGASASMAVDAV | | | | | | | | | | | | |
| | AEPANAVK.G | | | | | | | | | | | | |
| 1066 | K.GGSSEPGTELQTT | | 1154.211 | 3 | 3460.619 | 3459.618 | 1 | −0.6 | 248 | 307.6 | 300.3 | 300.3 | 3.33 |
| | EQQAGASASMAVDAV | | | | | | | | | | | | |
| | AEPANAVK.G | | | | | | | | | | | | |
| 1725 | R.MKQPPATTPPFKT | C[+57] | 809.732 | 3 | 2427.180 | 2427.180 | 0 | 0.4 | 296 | 651.1 | 651.1 | 651.1 | 10.08 |
| | HC[+57.021]QEAE | | | | | | | | | | | | |
| | TK.T | | | | | | | | | | | | |
| 1725 | R.MKQPPATTPPFKT | C[+57] | 607.551 | 4 | 2427.181 | 2427.180 | 0 | 0.6 | 296 | 614.1 | 614.1 | 614.1 | 10.46 |
| | HC[+57.021]QEAE | | | | | | | | | | | | |
| | TK.T | | | | | | | | | | | | |
| 824 | R.MKQPPATTPPFK. | | 671.864 | 2 | 1342.721 | 1342.719 | 0 | 1.3 | 296 | 559.4 | 559.4 | 559.4 | 7.59 |
| | T | | | | | | | | | | | | |
| 1666 | R.M[+15.995]KQP | C[+57], | 611.549 | 4 | 2443.175 | 2443.174 | 0 | 0.0 | 296 | 538.4 | 538.4 | 538.4 | 8.62 |
| | PATTPPFKTHC | M[+16] | | | | | | | | | | | |
| | [+57.021]QEA | | | | | | | | | | | | |
| | ETK.T | | | | | | | | | | | | |
| 1725 | R.MKQPPATTPPFKT | C[+57] | 607.550 | 4 | 2427.180 | 2427.180 | 0 | 0.2 | 296 | 537.0 | 537.0 | 537.0 | 8.62 |
| | HC[+57.021]QEAE | | | | | | | | | | | | |
| | TK.T | | | | | | | | | | | | |
| 1725 | R.MKQPPATTPPFKT | C[+57] | 486.242 | 5 | 2427.181 | 2427.180 | 0 | 0.5 | 296 | 530.7 | 530.7 | 530.7 | 8.62 |
| | HC[+57.021]QEAE | | | | | | | | | | | | |
| | TK.T | | | | | | | | | | | | |
| 1725 | R.MKQPPATTPPFKT | C[+57] | 486.242 | 5 | 2427.183 | 2427.180 | 0 | 1.5 | 296 | 513.6 | 513.6 | 513.6 | 7.68 |
| | HC[+57.021]QEAE | | | | | | | | | | | | |
| | TK.T | | | | | | | | | | | | |
| 1725 | R.MKQPPATTPPFKT | C[+57] | 486.242 | 5 | 2427.181 | 2427.180 | 0 | 0.7 | 296 | 511.5 | 511.5 | 511.5 | 8.41 |
| | HC[+57.021]QEAE | | | | | | | | | | | | |
| | TK.T | | | | | | | | | | | | |
| 1725 | R.MKQPPATTPPFKT | C[+57] | 486.243 | 5 | 2427.185 | 2427.180 | 0 | 2.0 | 296 | 508.3 | 508.3 | 508.3 | 8.41 |
| | HC[+57.021]QEAE | | | | | | | | | | | | |
| | TK.T | | | | | | | | | | | | |
| 1666 | R.M[+15.995]KQP | C[+57], | 489.441 | 5 | 2443.174 | 2443.174 | 0 | −0.1 | 296 | 462.5 | 462.5 | 462.5 | 8.17 |
| | PATTPPFKTHC | M[+16] | | | | | | | | | | | |
| | [+57.021] | | | | | | | | | | | | |
| | QEAETK.T | | | | | | | | | | | | |
| 824 | R.MKQPPATTPPFK. | | 448.245 | 3 | 1342.719 | 1342.719 | 0 | 0.1 | 296 | 446.4 | 446.4 | 446.4 | 8.11 |
| | T | | | | | | | | | | | | |
| 824 | R.MKQPPATTPPFK. | | 448.244 | 3 | 1342.718 | 1342.719 | 1 | −0.3 | 296 | 407.7 | 407.7 | 407.7 | 7.03 |
| | T | | | | | | | | | | | | |
| 1666 | R.M[+15.995]KQP | C[+57], | 611.549 | 4 | 2443.175 | 2443.174 | 0 | 0.3 | 296 | 404.1 | 404.1 | 404.1 | 7.33 |
| | PATTPPFKTHC | M[+16] | | | | | | | | | | | |
| | [+57.021] | | | | | | | | | | | | |
| | QEAETK.T | | | | | | | | | | | | |
| 1539 | R.M[+15.995]KQP | M[+16] | 453.576 | 3 | 1358.713 | 1358.714 | 0 | −0.3 | 296 | 400.4 | 276.2 | 276.2 | 5.27 |
| | PATTPPFK.T | | | | | | | | | | | | |
| 1666 | R.M[+15.995]KQP | C[+57], | 489.440 | 5 | 2443.173 | 2443.174 | 0 | 0.7 | 296 | 388.1 | 388.1 | 388.1 | 7.24 |
| | PATTPPFKTHC | M[+16] | | | | | | | | | | | |
| | [+57.021] | | | | | | | | | | | | |
| | QEAETK.T | | | | | | | | | | | | |
| 824 | R.MKQPPATTPPFK. | | 448.245 | 3 | 1342.719 | 1342.719 | 0 | 0.5 | 296 | 369.4 | 369.4 | 369.4 | 6.68 |
| | T | | | | | | | | | | | | |
| 824 | R.MKQPPATTPPFK. | | 672.365 | 2 | 1343.723 | 1342.719 | 1 | 0.7 | 296 | 359.7 | 359.7 | 359.7 | 3.41 |
| | T | | | | | | | | | | | | |
| 1539 | R.M[+15.995]KQP | M[+16] | 453.576 | 3 | 1358.714 | 1358.714 | 0 | 0.5 | 296 | 355.8 | 229.6 | 229.6 | 5.16 |
| | PATTPPFK.T | | | | | | | | | | | | |
| 1666 | R.M[+15.995]KQP | C[+57], | 611.549 | 4 | 2443.175 | 2443.174 | 0 | 0.1 | 296 | 337.7 | 337.7 | 337.7 | 5.35 |
| | PATTPPFKTHC | M[+16] | | | | | | | | | | | |
| | [+57.021]QEAET | | | | | | | | | | | | |
| | K.T | | | | | | | | | | | | |
| 1539 | R.M[+15.995]KQP | M[+16] | 679.860 | 2 | 1358.713 | 1358.714 | 0 | 0.5 | 296 | 332.0 | 308.1 | 308.1 | 4.28 |
| | PATTPPFK.T | | | | | | | | | | | | |
| 1539 | R.M[+15.995]KQP | M[+16] | 453.576 | 3 | 1358.714 | 1358.714 | 0 | 0.5 | 296 | 313.8 | 144.3 | 144.3 | 4.41 |
| | PATTPPFK.T | | | | | | | | | | | | |
| 1687 | K.QPPATTPPFKTHC | C[+57] | 542.767 | 4 | 2168.045 | 2168.044 | 0 | 0.3 | 298 | 507.9 | 507.9 | 507.9 | 8.72 |
| | [+57.021]QEAETK | | | | | | | | | | | | |
| | .T | | | | | | | | | | | | |
| 1687 | K.QPPATTPPFKTHC | C[+57] | 542.766 | 4 | 2168.044 | 2168.044 | 0 | −0.2 | 298 | 441.2 | 441.2 | 441.2 | 7.75 |
| | [+57.021]QEAETK | | | | | | | | | | | | |
| | .T | | | | | | | | | | | | |
| 1687 | K.QPPATTPPFKTHC | C[+57] | 542.767 | 4 | 2168.044 | 2168.044 | 0 | 0.0 | 298 | 412.7 | 412.7 | 412.7 | 7.64 |
| | [+57.021]QEAETK | | | | | | | | | | | | |
| | .T | | | | | | | | | | | | |
| 1690 | K.THC[+57.021]Q | C[+57] | 769.679 | 3 | 2307.024 | 2307.023 | 0 | 0.3 | 308 | 643.7 | 643.7 | 643.7 | 9.08 |
| | EAETKTKDEMAAAEE | | | | | | | | | | | | |
| | K.V | | | | | | | | | | | | |
| 1690 | K.THC[+57.021]Q | C[+57] | 577.511 | 4 | 2307.022 | 2307.023 | 0 | −0.5 | 308 | 570.8 | 570.8 | 570.8 | 8.75 |
| | EAETKTKDEMAAAEE | | | | | | | | | | | | |
| | K.V | | | | | | | | | | | | |
| 1667 | K.THC[+57.021]Q | C[+57], | 581.510 | 4 | 2323.019 | 2323.018 | 0 | 0.4 | 308 | 320.3 | 320.3 | 320.3 | 5.35 |
| | EAETKTKDEM | M[+16] | | | | | | | | | | | |
| | [+15.995] | | | | | | | | | | | | |
| | AAAEEK.V | | | | | | | | | | | | |
| 1357 | K.TKDEMAAAEEKVG | | 502.921 | 3 | 1506.747 | 1506.747 | 0 | 0.0 | 317 | 716.5 | 628.2 | 628.2 | 12.46 |
| | K.N | | | | | | | | | | | | |
| 2203 | K.NQEADVQEVK.A | | 580.283 | 2 | 1159.559 | 1159.559 | 0 | 0.1 | 360 | 409.2 | 385.5 | 385.5 | 6.99 |
| 994 | K.SSLLGSGDWHQYY | | 903.768 | 3 | 2709.289 | 2709.288 | 0 | 0.6 | 484 | 708.6 | 708.6 | 708.6 | 11.58 |
| | DIVYMKPHGR.L | | | | | | | | | | | | |
| 994 | K.SSLLGSGDWHQYY | | 542.663 | 5 | 2709.288 | 2709.288 | 0 | 0.0 | 484 | 608.6 | 608.6 | 608.6 | 10.96 |
| | DIVYMKPHGR.L | | | | | | | | | | | | |
| 994 | K.SSLLGSGDWHQYY | | 542.664 | 5 | 2709.289 | 2709.288 | 0 | 0.3 | 484 | 542.1 | 542.1 | 542.1 | 9.12 |
| | DIVYMKPHGR.L | | | | | | | | | | | | |
| 1590 | K.SSLLGSGDWHQYY | M[+16] | 545.863 | 5 | 2725.284 | 2725.283 | 0 | 0.4 | 484 | 506.5 | 506.5 | 506.5 | 8.18 |
| | DIVYM[+15.995]K | | | | | | | | | | | | |
| | PHGR.L | | | | | | | | | | | | |
| 1590 | K.SSLLGSGDWHQYY | M[+16] | 545.863 | 5 | 2725.284 | 2725.283 | 0 | 0.4 | 484 | 478.2 | 478.2 | 478.2 | 7.94 |
| | DIVYM[+15.995]K | | | | | | | | | | | | |
| | PHGR.L | | | | | | | | | | | | |
| 994 | K.SSLLGSGDWHQYY | | 542.664 | 5 | 2709.289 | 2709.288 | 0 | 0.3 | 484 | 394.0 | 394.0 | 394.0 | 7.24 |
| | DIVYMKPHGR.L | | | | | | | | | | | | |
| 3593 | R.LQKVMNHITDGPR | | 503.605 | 3 | 1508.800 | 1508.800 | 0 | −0.2 | 507 | 453.3 | 391.2 | 391.2 | 7.13 |
| | .K | | | | | | | | | | | | |
| 2034 | R.LQKVMNHITDGPR | | 409.980 | 4 | 1636.898 | 1636.895 | 0 | 1.7 | 507 | 373.2 | 373.2 | 373.2 | 6.97 |
| | K.D | | | | | | | | | | | | |
| 860 | K.VMNHITDGPRKDL | | 574.982 | 3 | 1722.932 | 1722.932 | 0 | −0.1 | 510 | 482.2 | 482.2 | 482.2 | 6.06 |
| | VK.G | | | | | | | | | | | | |
| 860 | K.VMNHITDGPRKDL | | 431.489 | 4 | 1722.935 | 1722.932 | 0 | 2.0 | 510 | 450.9 | 450.9 | 450.9 | 7.44 |
| | VK.G | | | | | | | | | | | | |
| 860 | K.VMNHITDGPRKDL | | 431.489 | 4 | 1722.933 | 1722.932 | 0 | 0.3 | 510 | 442.9 | 442.9 | 442.9 | 7.44 |
| | VK.G | | | | | | | | | | | | |
| 860 | K.VMNHITDGPRKDL | | 574.982 | 3 | 1722.932 | 1722.932 | 0 | 0.0 | 510 | 431.6 | 431.6 | 431.6 | 5.71 |
| | VK.G | | | | | | | | | | | | |
| 860 | K.VMNHITDGPRKDL | | 431.489 | 4 | 1722.934 | 1722.932 | 0 | 1.0 | 510 | 419.4 | 419.4 | 419.4 | 7.09 |
| | VK.G | | | | | | | | | | | | |
| 1526 | K.VM[+15.995]NH | M[+16] | 580.314 | 3 | 1738.928 | 1738.927 | 0 | 0.6 | 510 | 379.7 | 379.7 | 379.7 | 5.36 |
| | ITDGPRKDLVK.G | | | | | | | | | | | | |
| 860 | K.VMNHITDGPRKDL | | 431.489 | 4 | 1722.932 | 1722.932 | 0 | 0.1 | 510 | 368.3 | 368.3 | 368.3 | 6.97 |
| | VK.G | | | | | | | | | | | | |
| 1526 | K.VM[+15.995]NH | M[+16] | 435.487 | 4 | 1738.928 | 1738.927 | 0 | 0.5 | 510 | 362.9 | 362.9 | 362.9 | 6.97 |
| | ITDGPRKDLVK.G | | | | | | | | | | | | |
| 860 | K.VMNHITDGPRKDL | | 431.489 | 4 | 1722.933 | 1722.932 | 0 | 0.5 | 510 | 341.0 | 341.0 | 341.0 | 5.58 |
| | VK.G | | | | | | | | | | | | |
| 860 | K.VMNHITDGPRKDL | | 431.489 | 4 | 1722.933 | 1722.932 | 0 | 0.5 | 510 | 329.2 | 329.2 | 329.2 | 5.35 |
| | VK.G | | | | | | | | | | | | |
| 1526 | K.VM[+15.995]NH | M[+16] | 435.487 | 4 | 1738.927 | 1738.927 | 0 | 0.0 | 510 | 310.9 | 310.9 | 310.9 | 5.11 |
| | ITDGPRKDLVK.G | | | | | | | | | | | | |
| 1834 | K.RYLWQHLKK.H | | 424.584 | 3 | 1271.738 | 1271.737 | 0 | 0.5 | 588 | 432.2 | 432.2 | 432.2 | 5.65 |
| 907 | R.YLWQHLKK.H | | 558.322 | 2 | 1115.636 | 1115.636 | 0 | −0.2 | 589 | 506.1 | 506.1 | 506.1 | 5.77 |
| 2363 | R.YLWQHLK.K | | 494.274 | 2 | 987.541 | 987.541 | 0 | 0.1 | 589 | 463.5 | 463.5 | 463.5 | 6.94 |
| 907 | R.YLWQHLKK.H | | 558.322 | 2 | 1115.636 | 1115.636 | 0 | 0.1 | 589 | 427.1 | 427.1 | 427.1 | 15.42 |
| 907 | R.YLWQHLKK.H | | 558.322 | 2 | 1115.636 | 1115.636 | 0 | 0.3 | 589 | 348.7 | 348.7 | 348.7 | 13.05 |
| 956 | K.HVVPLPDGK.S | | 481.277 | 2 | 961.546 | 961.547 | 0 | 0.2 | 597 | 535.6 | 358.9 | 358.9 | 6.74 |
| 956 | K.HVVPLPDGK.S | | 481.277 | 2 | 961.546 | 961.547 | 0 | 0.2 | 597 | 449.6 | 351.9 | 351.9 | 6.04 |
| 4624 | K.HVVPLPDGKSTDF | C[+57] | 587.807 | 4 | 2348.207 | 2348.207 | 0 | 0.1 | 597 | 337.8 | 337.8 | 337.8 | 5.66 |
| | LPVDC[+57.021]P | | | | | | | | | | | | |
| | VR.S | | | | | | | | | | | | |
| 4624 | K.HVVPLPDGKSTDF | C[+57] | 587.807 | 4 | 2348.208 | 2348.207 | 0 | 0.4 | 597 | 328.8 | 328.8 | 328.8 | 5.51 |
| | LPVDC[+57.021]P | | | | | | | | | | | | |
| | VR.S | | | | | | | | | | | | |
| 1704 | K.STDFLPVDC | C[+57] | 703.343 | 2 | 1405.678 | 1405.678 | 0 | 0.3 | 606 | 588.8 | 557.3 | 557.3 | 9.59 |
| | [+57.021]PVR.S | | | | | | | | | | | | |
| 1704 | K.STDFLPVDC | C[+57] | 469.231 | 3 | 1405.677 | 1405.678 | 0 | −0.5 | 606 | 467.8 | 467.8 | 467.8 | 5.79 |
| | [+57.021] | | | | | | | | | | | | |
| | PVR.S | | | | | | | | | | | | |
| 812 | R.DLSHILGIPQSW | | 507.944 | 3 | 1521.817 | 1521.817 | 0 | 0.3 | 661 | 451.2 | 451.2 | 451.2 | 6.62 |
| | R.L | | | | | | | | | | | | |
| 1133 | R.QPEDTWAALEGLS | | 933.453 | 4 | 3730.792 | 3730.788 | 0 | 1.0 | 714 | 777.8 | 777.8 | 777.8 | 12.79 |
| | FSPFREQMLDTSSLL | | | | | | | | | | | | |
| | QFMR.E | | | | | | | | | | | | |
| 1616 | R.QPEDTWAALEGLS | M[+16] | 1249.602 | 3 | 3746.790 | 3746.783 | 0 | 2.0 | 714 | 732.3 | 676.3 | 91.3 | 12.39 |
| | FSPFREQM | | | | | | | | | | | | |
| | [+15.995]LDTSS | | | | | | | | | | | | |
| | LLQFMR.E | | | | | | | | | | | | |
| 1133 | R.QPEDTWAALEGLS | | 1244.271 | 3 | 3730.797 | 3730.788 | 0 | 2.5 | 714 | 508.9 | 508.9 | 508.9 | 18.34 |
| | FSPFREQMLDTSSLL | | | | | | | | | | | | |
| | QFMR.E | | | | | | | | | | | | |
| 885 | R.QPEDTWAALEGLS | | 684.334 | 3 | 2050.988 | 2050.987 | 0 | 0.5 | 714 | 491.8 | 491.8 | 491.8 | 18.32 |
| | FSPFR.E | | | | | | | | | | | | |
| 949 | R.EQMLDTSSLLQFM | | 849.913 | 2 | 1698.819 | 1698.819 | 0 | 0.1 | 732 | 595.8 | 595.8 | 595.8 | 9.96 |
| | R.E | | | | | | | | | | | | |
| 949 | R.EQMLDTSSLLQFM | | 566.945 | 3 | 1698.819 | 1698.819 | 0 | 0.2 | 732 | 523.5 | 523.5 | 523.5 | 18.53 |
| | R.E | | | | | | | | | | | | |
| 1558 | R.EQM[+15.995]L | M[+16] | 572.276 | 3 | 1714.814 | 1714.814 | 0 | 0.1 | 732 | 507.7 | 507.7 | 507.7 | 7.59 |
| | DTSSLLQFMR.E | | | | | | | | | | | | |
| 1860 | R.EKQHLLSIDEPLF | | 575.649 | 3 | 1724.933 | 1724.933 | 0 | 0.0 | 746 | 409.3 | 409.3 | 409.3 | 7.64 |
| | R.S | | | | | | | | | | | | |
| 1860 | R.EKQHLLSIDEPLF | | 431.989 | 4 | 1724.933 | 1724.933 | 0 | 0.0 | 746 | 371.4 | 371.4 | 371.4 | 7.28 |
| | R.S | | | | | | | | | | | | |
| 2190 | K.QHLLSIDEPLFR. | | 489.937 | 3 | 1467.796 | 1467.795 | 0 | 0.0 | 748 | 437.6 | 437.6 | 437.6 | 7.22 |
| | S | | | | | | | | | | | | |
| 1074 | R.LLSLVDSAGQR.D | | 579.828 | 2 | 1158.648 | 1158.648 | 0 | −0.1 | 881 | 568.8 | 497.1 | 497.1 | 9.59 |
| 1226 | R.DETGNNSVQTVFQ | | 746.377 | 3 | 2237.116 | 2237.116 | 0 | 0.2 | 892 | 601.6 | 601.6 | 601.6 | 10.77 |
| | GTLAATKR.W | | | | | | | | | | | | |
| 867 | R.LVEIQFPAEHGWK | | 693.347 | 4 | 2770.366 | 2770.366 | 0 | 0.2 | 942 | 552.4 | 552.4 | 552.4 | 8.93 |
| | ESLLGDMEWR.L | | | | | | | | | | | | |
| 867 | R.LVEIQFPAEHGWK | | 693.347 | 4 | 2770.365 | 2770.366 | 0 | −0.3 | 942 | 482.8 | 482.8 | 482.8 | 8.72 |
| | ESLLGDMEWR.L | | | | | | | | | | | | |
| 4202 | K.ESLLGDMEWR.L | | 618.290 | 2 | 1235.573 | 1235.573 | 0 | 0.3 | 955 | 341.0 | 341.0 | 341.0 | 5.48 |
| 996 | K.GLEDSVAKTFEK. | | 662.344 | 2 | 1323.680 | 1323.679 | 0 | 0.6 | 986 | 530.3 | 327.7 | 327.7 | 6.55 |
| | C | | | | | | | | | | | | |
| 996 | K.GLEDSVAKTFEK. | | 441.898 | 3 | 1323.679 | 1323.679 | 0 | −0.1 | 986 | 410.0 | 309.1 | 309.1 | 5.59 |
| | C | | | | | | | | | | | | |
| 972 | K.FGIVLSAVITK.S | | 574.358 | 2 | 1147.709 | 1147.709 | 0 | 0.2 | 1025 | 533.4 | 533.4 | 533.4 | 8.02 |
| 913 | R.TADNFDDILKHLL | | 714.720 | 3 | 2142.146 | 2142.144 | 0 | 0.9 | 1040 | 659.2 | 659.2 | 659.2 | 11.77 |
| | TLADVK.H | | | | | | | | | | | | |
| 913 | R.TADNFDDILKHLL | | 536.292 | 4 | 2142.146 | 2142.144 | 0 | 0.6 | 1040 | 575.3 | 575.3 | 575.3 | 9.77 |
| | TLADVK.H | | | | | | | | | | | | |
| 913 | R.TADNFDDILKHLL | | 536.292 | 4 | 2142.146 | 2142.144 | 0 | 0.8 | 1040 | 574.9 | 574.9 | 574.9 | 9.77 |
| | TLADVK.H | | | | | | | | | | | | |
| 913 | R.TADNFDDILKHLL | | 536.292 | 4 | 2142.145 | 2142.144 | 0 | 0.5 | 1040 | 545.3 | 545.3 | 545.3 | 8.93 |
| | TLADVK.H | | | | | | | | | | | | |
| 817 | R.TADNFDDILK.H | | 576.283 | 2 | 1151.558 | 1151.558 | 0 | 0.1 | 1040 | 490.9 | 490.9 | 490.9 | 7.57 |
| 913 | R.TADNFDDILKHLL | | 714.721 | 3 | 2142.147 | 2142.144 | 0 | 1.3 | 1040 | 419.6 | 1419.6 | 419.6 | 7.40 |
| | TLADVK.H | | | | | | | | | | | | |
| 1060 | K.HLLTLADVK.H | | 505.306 | 2 | 1009.604 | 1009.604 | 0 | −0.1 | 1050 | 581.0 | 581.0 | 581.0 | 18.86 |
| 1724 | R.LC[+57.021]GT | C[+57] | 509.014 | 4 | 2033.034 | 2033.033 | 0 | 0.2 | 1063 | 643.8 | 625.8 | 625.8 | 10.46 |
| | DEKILANVTEDAKR. | | | | | | | | | | | | |
| | L | | | | | | | | | | | | |
| 1724 | R.LC[+57.021]GT | C[+57] | 509.014 | 4 | 2033.034 | 2033.033 | 0 | 0.3 | 1063 | 427.4 | 427.4 | 427.4 | 7.09 |
| | DEKILANVTEDAKR. | | | | | | | | | | | | |
| | L | | | | | | | | | | | | |
| 1724 | R.LC[+57.021]GT | C[+57] | 509.014 | 4 | 2033.034 | 2033.033 | 0 | 0.5 | 1063 | 311.2 | 311.2 | 311.2 | 5.27 |
| | DEKILANVTEDAKR. | | | | | | | | | | | | |
| | L | | | | | | | | | | | | |
| 951 | K.ILANVTEDAKR.L | | 615.346 | 2 | 1229.684 | 1229.685 | 0 | 0.7 | 1070 | 559.2 | 559.2 | 559.2 | 7.13 |
| 951 | K.ILANVTEDAKR.L | | 410.567 | 3 | 1229.685 | 1229.685 | 0 | 0.1 | 1070 | 530.2 | 1530.2 | 530.2 | 18.56 |
| 829 | K.ILANVTEDAK.R | | 537.295 | 2 | 1073.583 | 1073.584 | 0 | 0.4 | 1070 | 478.3 | 301.6 | 301.6 | 5.19 |
| 951 | K.ILANVTEDAKR.L | | 410.567 | 3 | 1229.685 | 1229.685 | 0 | 0.0 | 1070 | 464.0 | 464.0 | 464.0 | 7.38 |
| 951 | K.ILANVTEDAKR.L | | 410.566 | 3 | 1229.685 | 1229.685 | 0 | 0.2 | 1070 | 362.5 | 289.6 | 289.6 | 15.25 |
| 951 | K.ILANVTEDAKR.L | | 615.346 | 2 | 1229.685 | 1229.685 | 0 | 0.1 | 1070 | 340.7 | 340.7 | 340.7 | 5.52 |
| 1772 | K.RLIAVADSVLTK. | | 643.396 | 2 | 1285.784 | 1285.784 | 0 | 0.4 | 1080 | 411.9 | 411.9 | 411.9 | 6.83 |
| | V | | | | | | | | | | | | |
| 2210 | R.LIAVADSVLTK.V | | 565.345 | 2 | 1129.683 | 1129.683 | 0 | 0.1 | 1081 | 461.3 | 461.3 | 461.3 | 7.10 |
| 1220 | K.VVGDLLSGTILVG | | 1040.636 | 2 | 2080.264 | 2080.263 | 0 | 0.5 | 1092 | 634.4 | 634.4 | 634.4 | 10.96 |
| | QLELIIK.H | | | | | | | | | | | | |
| 1220 | K.VVGDLLSGTILVG | | 694.093 | 3 | 2080.265 | 2080.263 | 0 | 0.9 | 1092 | 613.1 | 613.1 | 613.1 | 10.37 |
| | QLELIIK.H | | | | | | | | | | | | |
| 2369 | K.NQFLDIWQLREK. | | 530.620 | 3 | 1589.844 | 1589.844 | 0 | 0.3 | 1114 | 451.7 | 451.7 | 451.7 | 7.38 |
| | S | | | | | | | | | | | | |
| 2135 | K.NQFLDIWQLR.E | | 444.907 | 3 | 1332.707 | 1332.706 | 0 | 0.4 | 1114 | 435.0 | 435.0 | 435.0 | 6.62 |
| 2135 | K.NQFLDIWQLR.E | | 666.857 | 2 | 1332.707 | 1332.706 | 0 | 0.7 | 1114 | 361.9 | 361.9 | 361.9 | 6.72 |
| 1729 | K.SLSPQDEQC | C[+57] | 1066.982 | 2 | 2132.957 | 2132.955 | 0 | 0.6 | 1126 | 513.4 | 513.4 | 513.4 | 8.91 |
| | [+57.021]AVEEAL | | | | | | | | | | | | |
| | DWR.R | | | | | | | | | | | | |
| 4626 | K.SLSPQDEQC | C[+57] | 763.691 | 3 | 2289.058 | 2289.056 | 0 | 0.7 | 1126 | 382.1 | 382.1 | 382.1 | 7.05 |
| | [+57.021]AVEEAL | | | | | | | | | | | | |
| | DWRR.E | | | | | | | | | | | | |
| 1763 | R.REELLLLK.K | | 507.321 | 2 | 1013.635 | 1013.635 | 0 | −0.4 | 1144 | 375.7 | 375.7 | 375.7 | 5.83 |
| 989 | R.EELLLLKK.E | | 493.318 | 2 | 985.629 | 985.629 | 0 | 0.1 | 1145 | 550.1 | 550.1 | 550.1 | 7.83 |
| 989 | R.EELLLLKK.E | | 493.318 | 2 | 985.629 | 985.629 | 0 | 0.0 | 1145 | 540.7 | 540.7 | 540.7 | 8.56 |
| 2221 | R.EELLLLK.K | | 429.271 | 2 | 857.535 | 857.534 | 0 | 0.4 | 1145 | 465.3 | 465.3 | 465.3 | 7.33 |
| 989 | R.EELLLLKK.E | | 493.318 | 2 | 985.629 | 985.629 | 0 | −0.3 | 1145 | 383.7 | 383.7 | 383.7 | 6.52 |
| 1703 | R.C[+57.021]VDS | C[+57] | 417.723 | 2 | 834.439 | 834.439 | 0 | 0.3 | 1156 | 506.0 | 506.0 | 506.0 | 7.18 |
| | LLK.M | | | | | | | | | | | | |
| 1703 | R.C[+57.021]VDS | C[+57] | 417.723 | 2 | 834.439 | 834.439 | 0 | 0.0 | 1156 | 463.6 | 463.6 | 463.6 | 6.94 |
| | LLK.M | | | | | | | | | | | | |
| 1703 | R.C[+57.021]VDS | C[+57] | 417.723 | 2 | 834.439 | 834.439 | 0 | 0.2 | 1156 | 350.1 | 182.9 | 182.9 | 4.86 |
| | LLK.M | | | | | | | | | | | | |
| 926 | K.HLIQVDFGVLAVR | | 733.928 | 2 | 1466.848 | 1466.848 | 0 | −0.1 | 1169 | 561.9 | 561.9 | 561.9 | 9.59 |
| | .H | | | | | | | | | | | | |
| 926 | K.HLIQVDFGVLAVR | | 489.621 | 3 | 1466.847 | 1466.848 | 0 | −0.6 | 1169 | 518.8 | 518.8 | 518.8 | 7.83 |
| | .H | | | | | | | | | | | | |
| 926 | K.HLIQVDFGVLAVR | | 489.620 | 3 | 1466.846 | 1466.848 | 0 | 1.1 | 1169 | 421.0 | 421.0 | 421.0 | 6.75 |
| | .H | | | | | | | | | | | | |
| 1160 | R.HSQDLSSKRLNDT | | 489.762 | 4 | 1956.026 | 1956.026 | 0 | −0.1 | 1182 | 614.1 | 614.1 | 614.1 | 10.46 |
| | VTVR.L | | | | | | | | | | | | |
| 2114 | R.LNDTVTVR.L | | 459.256 | 2 | 917.505 | 917.505 | 0 | 0.2 | 1191 | 384.0 | 384.0 | 384.0 | 6.54 |
| 4627 | R.LSTSSNSQR.A | | 490.244 | 2 | 979.481 | 979.480 | 0 | 0.2 | 1199 | 380.0 | 380.0 | 380.0 | 6.72 |
| 4627 | R.LSTSSNSQR.A | | 490.244 | 2 | 979.480 | 979.480 | 0 | 0.0 | 1199 | 346.8 | 346.8 | 346.8 | 5.33 |
| 1917 | R.LSTSSNSQRATHY | | 687.585 | 4 | 2747.317 | 2747.317 | 0 | 0.0 | 1199 | 303.7 | 303.7 | 303.7 | 5.20 |
| | HLSSQVQEMAGK.I | | | | | | | | | | | | |
| 811 | R.ATHYHLSSQVQEM | | 600.064 | 4 | 2397.234 | 2397.234 | 0 | 0.0 | 1208 | 696.6 | 696.6 | 696.6 | 11.77 |
| | AGKIDLLR.D | | | | | | | | | | | | |
| 1012 | R.ATHYHLSSQVQEM | | 893.930 | 2 | 1786.853 | 1786.854 | 0 | −0.6 | 1208 | 696.2 | 696.2 | 696.2 | 9.88 |
| | AGK.I | | | | | | | | | | | | |
| 1012 | R.ATHYHLSSQVQEM | | 596.290 | 3 | 1786.854 | 1786.854 | 0 | −0.1 | 1208 | 674.9 | 674.9 | 674.9 | 11.96 |
| | AGK.I | | | | | | | | | | | | |
| 1012 | R.ATHYHLSSQVQEM | | 596.289 | 3 | 1786.854 | 1786.854 | 0 | 0.2 | 1208 | 634.9 | 634.9 | 634.9 | 10.96 |
| | AGK.I | | | | | | | | | | | | |
| 1012 | R.ATHYHLSSQVQEM | | 596.290 | 3 | 1786.855 | 1786.854 | 0 | 0.2 | 1208 | 587.1 | 587.1 | 587.1 | 9.96 |
| | AGK.I | | | | | | | | | | | | |
| 1562 | R.ATHYHLSSQVQEM | M[+16] | 601.621 | 3 | 1802.849 | 1802.849 | 0 | −0.2 | 1208 | 583.8 | 583.8 | 583.8 | 9.96 |
| | [+15.995]AGK.I | | | | | | | | | | | | |
| 1562 | R.ATHYHLSSQVQEM | M[+16] | 601.621 | 3 | 1802.849 | 1802.849 | 0 | 0.0 | 1208 | 564.9 | 564.9 | 564.9 | 9.96 |
| | [+15.995]AGK.I | | | | | | | | | | | | |
| 1012 | R.ATHYHLSSQVQEM | | 596.289 | 3 | 1786.853 | 1786.854 | 0 | −0.5 | 1208 | 529.4 | 529.4 | 529.4 | 7.69 |
| | AGK.I | | | | | | | | | | | | |
| 1562 | R.ATHYHLSSQVQEM | M[+16] | 601.621 | 3 | 1802.850 | 1802.849 | 0 | 0.4 | 1208 | 467.1 | 467.1 | 467.1 | 7.70 |
| | [+15.995]AGK.I | | | | | | | | | | | | |
| 811 | R.ATHYHLSSQVQEM | | 480.253 | 5 | 2397.234 | 2397.234 | 0 | −0.2 | 1208 | 462.2 | 462.2 | 462.2 | 8.48 |
| | AGKIDLLR.D | | | | | | | | | | | | |
| 811 | R.ATHYHLSSQVQEM | | 480.253 | 5 | 2397.234 | 2397.234 | 0 | 0.2 | 1208 | 452.2 | 452.2 | 452.2 | 7.75 |
| | AGKIDLLR.D | | | | | | | | | | | | |
| 1012 | R.ATHYHLSSQVQEM | | 447.470 | 4 | 1786.857 | 1786.854 | 0 | 1.6 | 1208 | 441.6 | 441.6 | 441.6 | 7.94 |
| | AGK.I | | | | | | | | | | | | |
| 1012 | R.ATHYHLSSQVQEM | | 447.469 | 4 | 1786.856 | 1786.854 | 0 | 1.0 | 1208 | 430.7 | 430.7 | 430.7 | 8.56 |
| | AGK.I | | | | | | | | | | | | |
| 1012 | R.ATHYHLSSQVQEM | | 447.469 | 4 | 1786.854 | 1786.854 | 0 | 0.0 | 1208 | 395.6 | 395.6 | 395.6 | 7.47 |
| | AGK.I | | | | | | | | | | | | |
| 1012 | R.ATHYHLSSQVQEM | | 447.469 | 4 | 1786.854 | 1786.854 | 0 | −0.2 | 1208 | 376.8 | 376.8 | 376.8 | 7.24 |
| | AGK.I | | | | | | | | | | | | |
| 1562 | R.ATHYHLSSQVQEM | M[+16] | 451.468 | 4 | 1802.848 | 1802.849 | 0 | −0.3 | 1208 | 374.7 | 374.7 | 374.7 | 6.77 |
| | [+15.995]AGK.I | | | | | | | | | | | | |
| 1012 | R.ATHYHLSSQVQEM | | 447.469 | 4 | 1786.854 | 1786.854 | 0 | 0.2 | 1208 | 370.1 | 370.1 | 370.1 | 7.47 |
| | AGK.I | | | | | | | | | | | | |
| 1012 | R.ATHYHLSSQVQEM | | 447.469 | 4 | 1786.856 | 1786.854 | 0 | 0.8 | 1208 | 366.9 | 366.9 | 366.9 | 7.72 |
| | AGK.I | | | | | | | | | | | | |
| 1012 | R.ATHYHLSSQVQEM | | 447.469 | 4 | 1786.855 | 1786.854 | 0 | 0.6 | 1208 | 364.9 | 364.9 | 364.9 | 7.47 |
| | AGK.I | | | | | | | | | | | | |
| 1562 | R.ATHYHLSSQVQEM | M[+16] | 451.468 | 4 | 1802.849 | 1802.849 | 0 | −0.1 | 1208 | 356.1 | 356.1 | 356.1 | 6.09 |
| | [+15.995]AGK.I | | | | | | | | | | | | |
| 1562 | R.ATHYHLSSQVQEM | M[+16] | 451.468 | 4 | 1802.850 | 1802.849 | 0 | 0.3 | 1208 | 344.9 | 344.9 | 344.9 | 5.86 |
| | [+15.995]AGK.I | | | | | | | | | | | | |
| 3483 | K.IDLLRDSHIFQLF | | 490.521 | 4 | 1959.061 | 1959.060 | 0 | 0.6 | 1224 | 356.2 | 356.2 | 356.2 | 5.89 |
| | WR.E | | | | | | | | | | | | |
| 838 | R.DSHIFQLFWR.E | | 674.844 | 2 | 1348.680 | 1348.680 | 0 | 0.2 | 1229 | 580.1 | 419.8 | 419.8 | 8.86 |
| 838 | R.DSHIFQLFWR.E | | 450.232 | 3 | 1348.680 | 1348.680 | 0 | 0.2 | 1229 | 500.4 | 500.4 | 500.4 | 7.81 |
| 1468 | R.EAAEPLSEPKEDQ | S[+80] | 1383.883 | 4 | 5532.510 | 5532.505 | 0 | 0.8 | 1239 | 770.3 | 762.8 | 45.5 | 12.66 |
| | EAAELLS | | | | | | | | | | | | |
| | [+79.966] | | | | | | | | | | | | |
| | EPEEESERHILELE | | | | | | | | | | | | |
| | EVYDYLYQPSYR.K | | | | | | | | | | | | |
| 1380 | R.EAAEPLSEPKEDQ | | 1363.893 | 4 | 5452.550 | 5452.539 | 0 | 1.9 | 1239 | 732.7 | 730.8 | 730.8 | 11.66 |
| | EAAELLSEPEEESER | | | | | | | | | | | | |
| | HILELEEVYDYLYQP | | | | | | | | | | | | |
| | SYR.K | | | | | | | | | | | | |
| 1380 | R.EAAEPLSEPKEDQ | | 1091.315 | 5 | 5452.548 | 5452.539 | 0 | 1.6 | 1239 | 724.6 | 724.6 | 724.6 | 11.66 |
| | EAAELLSEPEEESER | | | | | | | | | | | | |
| | HILELEEVYDYLYQP | | | | | | | | | | | | |
| | SYR.K | | | | | | | | | | | | |
| 1299 | R.EAAEPLSEPKEDQ | | 1047.813 | 3 | 3141.424 | 3141.423 | 0 | 0.4 | 1239 | 621.9 | 621.9 | 621.9 | 10.39 |
| | EAAELLSEPEEESER | | | | | | | | | | | | |
| | .H | | | | | | | | | | | | |
| 1469 | R.EAAEPLSEPKEDQ | S[+80] | 1107.308 | 5 | 5532.509 | 5532.505 | 0 | 0.6 | 1239 | 565.7 | 565.7 | 11.6 | 8.66 |
| | EAAELLSEPEEES | | | | | | | | | | | | |
| | [+79.966]ERHI | | | | | | | | | | | | |
| | LELE | | | | | | | | | | | | |
| | EVYDYLYQPSYR.K | | | | | | | | | | | | |
| 3065 | R.EAAEPLSEPKEDQ | Y[+80] | 1383.888 | 4 | 5532.529 | 5532.505 | 0 | 4.2 | 1239 | 537.2 | 375.6 | 10.9 | 5.22 |
| | EAAELLSEPEEESER | | | | | | | | | | | | |
| | HILELEEVY | | | | | | | | | | | | |
| | [+79.966] | | | | | | | | | | | | |
| | DYLYQPSYR.K | | | | | | | | | | | | |
| 1897 | R.KFIKLHQDLK.S | | 423.928 | 3 | 1269.768 | 1269.768 | 0 | 0.0 | 1285 | 449.5 | 449.5 | 449.5 | 5.75 |
| 1897 | R.KFIKLHQDLK.S | | 423.928 | 3 | 1269.768 | 1269.768 | 0 | 0.0 | 1285 | 316.5 | 316.5 | 316.5 | 3.67 |
| 2161 | K.DRVEQIKEYHHLH | | 402.382 | 6 | 2409.253 | 2409.253 | 0 | −0.3 | 1338 | 336.2 | 336.2 | 336.2 | 4.20 |
| | QAVHAAK.V | | | | | | | | | | | | |
| 1892 | R.VEQIKEYHHLHQA | | 428.431 | 5 | 2138.126 | 2138.125 | 0 | 0.4 | 1340 | 320.5 | 300.6 | 300.6 | 4.13 |
| | VHAAK.V | | | | | | | | | | | | |
| 1892 | R.VEQIKEYHHLHQA | | 428.431 | 5 | 2138.126 | 2138.125 | 0 | 0.5 | 1340 | 302.6 | 302.6 | 302.6 | 4.20 |
| | VHAAK.V | | | | | | | | | | | | |
| 946 | R.ETLDQINQELIQA | | 821.936 | 2 | 1642.865 | 1642.865 | 0 | 0.3 | 1391 | 705.7 | 705.7 | 705.7 | 12.96 |
| | K.K | | | | | | | | | | | | |
| 1077 | R.ETLDQINQELIQA | | 885.983 | 2 | 1770.959 | 1770.960 | 0 | 0.4 | 1391 | 586.9 | 559.6 | 559.6 | 9.06 |
| | KK.L | | | | | | | | | | | | |
| 1077 | R.ETLDQINQELIQA | | 590.991 | 3 | 1770.959 | 1770.960 | 0 | 0.5 | 1391 | 551.3 | 551.3 | 551.3 | 8.23 |
| | KK.L | | | | | | | | | | | | |
| 946 | R.ETLDQINQELIQA | | 821.936 | 2 | 1642.865 | 1642.865 | 0 | 0.2 | 1391 | 530.5 | 530.5 | 530.5 | 9.12 |
| | K.K | | | | | | | | | | | | |
| 946 | R.ETLDQINQELIQA | | 548.293 | 3 | 1642.865 | 1642.865 | 0 | 0.3 | 1391 | 513.9 | 513.9 | 513.9 | 7.59 |
| | K.K | | | | | | | | | | | | |
| 946 | R.ETLDQINQELIQA | | 821.936 | 2 | 1642.865 | 1642.865 | 0 | 0.2 | 1391 | 459.6 | 459.6 | 459.6 | 8.67 |
| | K.K | | | | | | | | | | | | |
| 1101 | K.KLLQDISEAR.C | | 586.835 | 2 | 1172.663 | 1172.663 | 0 | −0.5 | 1405 | 592.0 | 592.0 | 592.0 | 7.91 |
| 998 | K.LLQDISEAR.C | | 522.788 | 2 | 1044.568 | 1044.568 | 0 | −0.2 | 1406 | 549.9 | 283.8 | 283.8 | 6.35 |
| 998 | K.LLQDISEAR.C | | 522.788 | 2 | 1044.568 | 1044.568 | 0 | 0.0 | 1406 | 363.9 | 194.1 | 194.1 | 4.97 |
| 1029 | K.ALDKDQYLPRK.L | | 449.586 | 3 | 1346.743 | 1346.743 | 0 | 0.2 | 1498 | 558.3 | 558.3 | 558.3 | 7.52 |
| 2191 | K.ALDKDQYLPR.K | | 407.222 | 3 | 1219.651 | 1218.648 | 1 | 0.0 | 1498 | 458.7 | 458.7 | 458.7 | 5.65 |
| 2191 | K.ALDKDQYLPR.K | | 609.828 | 2 | 1218.648 | 1218.648 | 0 | 0.4 | 1498 | 437.3 | 437.3 | 437.3 | 6.80 |
| 832 | R.NLEWLKTVNESHG | | 500.256 | 4 | 1998.004 | 1998.004 | 0 | −0.2 | 1515 | 490.0 | 490.0 | 490.0 | 7.99 |
| | SVER.S | | | | | | | | | | | | |
| 832 | R.NLEWLKTVNESHG | | 500.257 | 4 | 1998.005 | 1998.004 | 0 | 0.3 | 1515 | 480.8 | 480.8 | 480.8 | 8.72 |
| | SVER.S | | | | | | | | | | | | |
| 1766 | R.NLEWLK.T | | 401.727 | 2 | 802.446 | 802.446 | 0 | 0.3 | 1515 | 310.0 | 210.0 | 210.0 | 5.01 |
| 1766 | R.NLEWLK.T | | 401.727 | 2 | 802.446 | 802.446 | 0 | 0.1 | 1515 | 300.9 | 229.0 | 229.0 | 5.01 |
| 2033 | K.TVNESHGSVER.S | | 405.531 | 3 | 1214.578 | 1214.576 | 0 | 1.3 | 1521 | 318.1 | 318.1 | 318.1 | 5.40 |
| 808 | R.SSLTLATAINQR. | | 637.857 | 2 | 1274.706 | 1274.706 | 0 | 0.1 | 1532 | 711.7 | 711.7 | 711.7 | 12.59 |
| | G | | | | | | | | | | | | |
| 808 | R.SSLTLATAINQR. | | 637.857 | 2 | 1274.707 | 1274.706 | 0 | 0.4 | 1532 | 668.6 | 668.6 | 668.6 | 10.86 |
| | G | | | | | | | | | | | | |
| 808 | R.SSLTLATAINQR. | | 637.857 | 2 | 1274.707 | 1274.706 | 0 | 0.1 | 1532 | 603.6 | 603.6 | 603.6 | 9.86 |
| | G | | | | | | | | | | | | |
| 808 | R.SSLTLATAINQR. | | 637.857 | 2 | 1274.706 | 1274.706 | 0 | 0.3 | 1532 | 556.2 | 1556.2 | 556.2 | 8.75 |
| | G | | | | | | | | | | | | |
| 808 | R.SSLTLATAINQR. | | 637.857 | 2 | 1274.707 | 1274.706 | 0 | 0.1 | 1532 | 508.2 | 508.2 | 508.2 | 7.81 |
| | G | | | | | | | | | | | | |
| 808 | R.SSLTLATAINQR. | | 425.574 | 3 | 1274.706 | 1274.706 | 0 | 0.1 | 1532 | 487.6 | 487.6 | 487.6 | 7.22 |
| | G | | | | | | | | | | | | |
| 808 | R.SSLTLATAINQR. | | 425.574 | 3 | 1274.706 | 1274.706 | 0 | 0.2 | 1532 | 470.6 | 470.6 | 470.6 | 6.97 |
| | G | | | | | | | | | | | | |
| 808 | R.SSLTLATAINQR. | | 637.857 | 2 | 1274.707 | 1274.706 | 0 | 0.5 | 1532 | 422.5 | 422.5 | 422.5 | 7.22 |
| | G | | | | | | | | | | | | |
| 808 | R.SSLTLATAINQR. | | 637.857 | 2 | 1274.707 | 1274.706 | 0 | 0.1 | 1532 | 416.3 | 416.3 | 416.3 | 7.46 |
| | G | | | | | | | | | | | | |
| 808 | R.SSLTLATAINQR. | | 637.857 | 2 | 1274.706 | 1274.706 | 0 | 0.2 | 1532 | 375.4 | 375.4 | 375.4 | 6.87 |
| | G | | | | | | | | | | | | |
| 813 | R.GIYVIQAPK.G | | 494.795 | 2 | 988.583 | 988.583 | 0 | 0.0 | 1544 | 476.8 | 387.2 | 387.2 | 7.33 |
| 1331 | K.ISPDTVLHLILPE | | 604.065 | 4 | 2413.236 | 2413.236 | 0 | 0.1 | 1557 | 687.9 | 687.9 | 687.9 | 11.96 |
| | SPGSHEESR.E | | | | | | | | | | | | |
| 937 | K.ISPDTVLHLILPE | | 997.769 | 4 | 3988.055 | 3988.055 | 0 | 0.1 | 1557 | 474.0 | 474.0 | 474.0 | 5.43 |
| | SPGSHEESREYSLEE | | | | | | | | | | | | |
| | VKELLNK.L | | | | | | | | | | | | |
| 937 | K.ISPDTVLHLILPE | | 997.770 | 4 | 3988.057 | 3988.055 | 0 | 0.5 | 1557 | 458.3 | 458.3 | 458.3 | 5.20 |
| | SPGSHEESREYSLEE | | | | | | | | | | | | |
| | VKELLNK.L | | | | | | | | | | | | |
| 937 | K.ISPDTVLHLILPE | | 665.516 | 6 | 3988.058 | 3988.055 | 0 | 0.8 | 1557 | 417.5 | 417.5 | 417.5 | 7.67 |
| | SPGSHEESREYSLEE | | | | | | | | | | | | |
| | VKELLNK.L | | | | | | | | | | | | |
| 937 | K.ISPDTVLHLILPE | | 665.516 | 6 | 3988.058 | 3988.055 | 0 | 0.6 | 1557 | 365.1 | 365.1 | 365.1 | 6.83 |
| | SPGSHEESREYSLEE | | | | | | | | | | | | |
| | VKELLNK.L | | | | | | | | | | | | |
| 937 | K.ISPDTVLHLILPE | | 665.516 | 6 | 3988.057 | 3988.055 | 0 | 0.5 | 1557 | 363.3 | 363.3 | 363.3 | 6.36 |
| | SPGSHEESREYSLEE | | | | | | | | | | | | |
| | VKELLNK.L | | | | | | | | | | | | |
| 937 | K.ISPDTVLHLILPE | | 798.417 | 5 | 3988.057 | 3988.055 | 0 | 0.4 | 1557 | 333.6 | 333.6 | 333.6 | 4.81 |
| | SPGSHEESREYSLEE | | | | | | | | | | | | |
| | VKELLNK.L | | | | | | | | | | | | |
| 937 | K.ISPDTVLHLILPE | | 798.417 | 5 | 3988.057 | 3988.055 | 0 | 0.5 | 1557 | 326.9 | 326.9 | 326.9 | 5.44 |
| | SPGSHEESREYSLEE | | | | | | | | | | | | |
| | VKELLNK.L | | | | | | | | | | | | |
| 982 | R.EYSLEEVKELLNK | | 531.950 | 3 | 1593.836 | 1593.837 | 0 | −0.4 | 1579 | 522.4 | 522.4 | 522.4 | 7.85 |
| | .L | | | | | | | | | | | | |
| 1718 | R.NNTEVERFSEVFC | C[+57] | 700.997 | 3 | 2100.977 | 2100.977 | 0 | 0.0 | 1602 | 498.1 | 498.1 | 498.1 | 7.99 |
| | [+57.021]SVQR.L | | | | | | | | | | | | |
| 1718 | R.NNTEVERFSEVFC | C[+57] | 700.997 | 3 | 2100.977 | 2100.977 | 0 | 0.2 | 1602 | 415.5 | 415.5 | 415.5 | 7.17 |
| | [+57.021]SVQR.L | | | | | | | | | | | | |
| 3334 | R.FSEVFC | C[+57] | 629.798 | 2 | 1258.588 | 1258.589 | 0 | −0.1 | 1609 | 544.1 | 544.1 | 544.1 | 7.78 |
| | [+57.021] | | | | | | | | | | | | |
| | SVQR.L | | | | | | | | | | | | |
| 928 | R.LSQAFIDLHSAGN | | 641.000 | 3 | 1920.985 | 1919.980 | 1 | 0.9 | 1619 | 492.9 | 492.9 | 492.9 | 8.91 |
| | MLFR.T | | | | | | | | | | | | |
| 1595 | R.LSQAFIDLHSAGN | M[+16] | 645.997 | 3 | 1935.975 | 1935.975 | 0 | 0.4 | 1619 | 407.2 | 407.2 | 407.2 | 7.59 |
| | M[+15.995]LFR.T | | | | | | | | | | | | |
| 899 | R.KQPPSDAALTMLS | | 582.987 | 3 | 1746.946 | 1746.946 | 0 | 0.2 | 1718 | 503.6 | 503.6 | 503.6 | 8.18 |
| | FIK.S | | | | | | | | | | | | |
| 899 | R.KQPPSDAALTMLS | | 582.987 | 3 | 1746.945 | 1746.946 | 0 | 0.6 | 1718 | 470.6 | 470.6 | 470.6 | 7.23 |
| | FIK.S | | | | | | | | | | | | |
| 899 | R.KQPPSDAALTMLS | | 582.987 | 3 | 1746.945 | 1746.946 | 0 | 0.3 | 1718 | 403.6 | 403.6 | 403.6 | 6.88 |
| | FIK.S | | | | | | | | | | | | |
| 899 | R.KQPPSDAALTMLS | | 873.976 | 2 | 1746.945 | 1746.946 | 0 | 0.6 | 1718 | 374.7 | 374.7 | 374.7 | 6.77 |
| | FIK.S | | | | | | | | | | | | |
| 899 | R.KQPPSDAALTMLS | | 873.976 | 2 | 1746.946 | 1746.946 | 0 | 0.2 | 1718 | 343.5 | 343.5 | 343.5 | 5.86 |
| | FIK.S | | | | | | | | | | | | |
| 899 | R.KQPPSDAALTMLS | | 873.977 | 2 | 1746.946 | 1746.946 | 0 | 0.3 | 1718 | 313.8 | 313.8 | 313.8 | 6.00 |
| | FIK.S | | | | | | | | | | | | |
| 809 | K.QPPSDAALTMLSF | | 540.289 | 3 | 1618.851 | 1618.851 | 0 | 0.3 | 1719 | 462.1 | 462.1 | 462.1 | 8.07 |
| | IK.S | | | | | | | | | | | | |
| 809 | K.QPPSDAALTMLSF | | 540.289 | 3 | 1618.851 | 1618.851 | 0 | 0.2 | 1719 | 455.9 | 455.9 | 455.9 | 7.10 |
| | IK.S | | | | | | | | | | | | |
| 809 | K.QPPSDAALTMLSF | | 540.288 | 3 | 1618.850 | 1618.851 | 0 | −0.4 | 1719 | 455.1 | 455.1 | 455.1 | 6.64 |
| | IK.S | | | | | | | | | | | | |
| 809 | K.QPPSDAALTMLSF | | 809.930 | 2 | 1618.853 | 1618.851 | 0 | 1.0 | 1719 | 434.7 | 434.7 | 434.7 | 8.56 |
| | IK.S | | | | | | | | | | | | |
| 809 | K.QPPSDAALTMLSF | | 809.929 | 2 | 1618.851 | 1618.851 | 0 | 0.2 | 1719 | 401.8 | 401.8 | 401.8 | 7.59 |
| | IK.S | | | | | | | | | | | | |
| 809 | K.QPPSDAALTMLSF | | 809.930 | 2 | 1618.852 | 1618.851 | 0 | 0.6 | 1719 | 400.3 | 400.3 | 400.3 | 7.59 |
| | IK.S | | | | | | | | | | | | |
| 1683 | K.SNC[+57.021]T | C[+57] | 411.884 | 3 | 1233.637 | 1233.637 | 0 | 0.1 | 1734 | 304.8 | 304.8 | 304.8 | 5.21 |
| | LRDVLR.A | | | | | | | | | | | | |
| 1312 | R.RVMEELPLMLLSE | | 630.350 | 4 | 2518.380 | 2518.377 | 0 | 1.1 | 1759 | 736.8 | 736.8 | 736.8 | 12.77 |
| | FSLVDKLR.I | | | | | | | | | | | | |
| 1312 | R.RVMEELPLMLLSE | | 840.132 | 3 | 2518.381 | 2518.377 | 0 | 1.5 | 1759 | 670.2 | 670.2 | 670.2 | 11.77 |
| | FSLVDKLR.I | | | | | | | | | | | | |
| 1633 | R.RVMEELPLM | M[+16] | 634.349 | 4 | 2534.375 | 2534.372 | 0 | 1.1 | 1759 | 652.7 | 652.7 | 346.7 | 11.77 |
| | [+15.995]LL | | | | | | | | | | | | |
| | SEFSLVDK | | | | | | | | | | | | |
| | LR.I | | | | | | | | | | | | |
| 1003 | R.RVMEELPLMLLSE | | 750.403 | 3 | 2249.193 | 2249.192 | 0 | 0.6 | 1759 | 526.7 | 526.7 | 526.7 | 9.12 |
| | FSLVDK.L | | | | | | | | | | | | |
| 1633 | R.RVMEELPLM | M[+16] | 845.463 | 3 | 2534.375 | 2534.372 | 0 | 1.1 | 1759 | 491.5 | 491.5 | 129.8 | 7.75 |
| | [+15.995]L | | | | | | | | | | | | |
| | LSEFSLVDK | | | | | | | | | | | | |
| | LR.I | | | | | | | | | | | | |
| 1593 | R.RVMEELPLM | M[+16] | 755.735 | 3 | 2265.189 | 2265.187 | 0 | 0.9 | 1759 | 366.2 | 366.2 | 167.8 | 7.47 |
| | [+15.995]L | | | | | | | | | | | | |
| | LSEFSLVDK | | | | | | | | | | | | |
| | .L | | | | | | | | | | | | |
| 1312 | R.RVMEELPLMLLSE | | 840.131 | 3 | 2518.378 | 2518.377 | 0 | 0.2 | 1759 | 340.6 | 340.6 | 340.6 | 5.66 |
| | FSLVDKLR.I | | | | | | | | | | | | |
| 862 | R.VMEELPLMLLSEF | | 788.098 | 3 | 2362.279 | 2362.276 | 0 | 1.1 | 1760 | 788.8 | 788.8 | 788.8 | 13.77 |
| | SLVDKLR.I | | | | | | | | | | | | |
| 1626 | R.VM[+15.995]EE | M[+16] | 793.428 | 3 | 2378.271 | 2378.271 | 0 | 0.2 | 1760 | 779.4 | 779.4 | 493.6 | 13.77 |
| | LPLMLLSEFSLVDKL | | | | | | | | | | | | |
| | R.I | | | | | | | | | | | | |
| 862 | R.VMEELPLMLLSEF | | 788.098 | 3 | 2362.278 | 2362.276 | 0 | 1.0 | 1760 | 738.7 | 738.7 | 738.7 | 12.77 |
| | SLVDKLR.I | | | | | | | | | | | | |
| 1606 | R.VMEELPLM | M[+16] | 793.429 | 3 | 2378.272 | 2378.271 | 0 | 0.4 | 1760 | 721.2 | 721.2 | 548.6 | 12.77 |
| | [+15.995]L | | | | | | | | | | | | |
| | LSEFSLVDKL | | | | | | | | | | | | |
| | R.I | | | | | | | | | | | | |
| 862 | R.VMEELPLMLLSEF | | 788.431 | 3 | 2363.279 | 2362.276 | 1 | 0.0 | 1760 | 705.0 | 705.0 | 705.0 | 12.77 |
| | SLVDKLR.I | | | | | | | | | | | | |
| 862 | R.VMEELPLMLLSEF | | 788.097 | 3 | 2362.278 | 2362.276 | 0 | 0.7 | 1760 | 691.5 | 691.5 | 691.5 | 11.77 |
| | SLVDKLR.I | | | | | | | | | | | | |
| 1606 | R.VMEELPLM | M[+16] | 793.430 | 3 | 2378.274 | 2378.271 | 0 | 1.4 | 1760 | 676.3 | 676.3 | 267.1 | 11.77 |
| | [+15.995]L | | | | | | | | | | | | |
| | LSEFSLVDKL | | | | | | | | | | | | |
| | R.I | | | | | | | | | | | | |
| 1626 | R.VM[+15.995]EE | M[+16] | 793.428 | 3 | 2378.271 | 2378.271 | 0 | 0.1 | 1760 | 671.1 | 671.1 | 400.2 | 11.77 |
| | LPLMLLSEFSLVDKL | | | | | | | | | | | | |
| | R.I | | | | | | | | | | | | |
| 1606 | R.VMEELPLM[+15. | M[+16] | 1189.640 | 2 | 2378.272 | 2378.271 | 0 | 0.6 | 1760 | 668.2 | 668.2 | 397.5 | 11.77 |
| | 995]LLSEFSLVDKL | | | | | | | | | | | | |
| | R.I | | | | | | | | | | | | |
| 1606 | R.VMEELPLM[+15. | M[+16] | 595.324 | 4 | 2378.273 | 2378.271 | 0 | 0.7 | 1760 | 644.3 | 644.3 | 282.9 | 10.17 |
| | 995]LLSEFSLVDKL | | | | | | | | | | | | |
| | R.I | | | | | | | | | | | | |
| 862 | R.VMEELPLMLLSEF | | 1181.641 | 2 | 2362.275 | 2362.276 | 0 | −0.5 | 1760 | 642.3 | 642.3 | 642.3 | 10.06 |
| | SLVDKLR.I | | | | | | | | | | | | |
| 1626 | R.VM[+15.995]EE | M[+16] | 793.428 | 3 | 2378.269 | 2378.271 | 0 | −0.9 | 1760 | 629.4 | 629.4 | 96.3 | 10.06 |
| | LPLMLLSEFSLVDKL | | | | | | | | | | | | |
| | R.I | | | | | | | | | | | | |
| 1082 | R.VMEELPLMLLSEF | | 1047.050 | 2 | 2093.093 | 2093.091 | 0 | 1.0 | 1760 | 624.2 | 624.2 | 624.2 | 10.96 |
| | SLVDK.L | | | | | | | | | | | | |
| 1606 | R.VMEELPLM[+15. | M[+16] | 793.430 | 3 | 2378.274 | 2378.271 | 0 | 1.4 | 1760 | 612.1 | 612.1 | 417.1 | 10.77 |
| | 995]LLSEFSLVDKL | | | | | | | | | | | | |
| | R.I | | | | | | | | | | | | |
| 1082 | R.VMEELPLMLLSEF | | 698.369 | 3 | 2093.093 | 2093.091 | 0 | 0.9 | 1760 | 576.2 | 576.2 | 576.2 | 9.37 |
| | SLVDK.L | | | | | | | | | | | | |
| 1614 | R.VMEELPLM[+15. | M[+16] | 703.700 | 3 | 2109.085 | 2109.086 | 0 | −0.2 | 1760 | 517.4 | 517.4 | 395.0 | 8.32 |
| | 995]LLSEFSLVDK. | | | | | | | | | | | | |
| | L | | | | | | | | | | | | |
| 1529 | R.VM[+15.995]EE | M[+16] | 1055.048 | 2 | 2109.088 | 2109.086 | 0 | 1.2 | 1760 | 501.4 | 414.2 | 1287.5 | 8.18 |
| | LPLMLLSEFSLVDK. | | | | | | | | | | | | |
| | L | | | | | | | | | | | | |
| 862 | R.VMEELPLMLLSEF | | 788.424 | 3 | 2363.258 | 2362.276 | 1 | −9.0 | 1760 | 454.3 | 454.3 | 454.3 | 5.82 |
| | SLVDKLR.I | | | | | | | | | | | | |
| 862 | R.VMEELPLMLLSEF | | 788.097 | 3 | 2362.277 | 2362.276 | 0 | 0.6 | 1760 | 129.4 | 429.4 | 429.4 | 7.40 |
| | SLVDKLR.I | | | | | | | | | | | | |
| 862 | R.VMEELPLMLLSEF | | 788.097 | 3 | 2362.277 | 2362.276 | 0 | 0.6 | 1760 | 420.9 | 420.9 | 420.9 | 7.40 |
| | SLVDKLR.I | | | | | | | | | | | | |
| 1614 | R.VMEELPLM[+15. | M[+16] | 1055.046 | 2 | 2109.085 | 2109.086 | 0 | −0.2 | 1760 | 384.6 | 384.6 | 234.1 | 7.72 |
| | 995]LLSEFSLVDK. | | | | | | | | | | | | |
| | L | | | | | | | | | | | | |
| 862 | R.VMEELPLMLLSEF | | 788.098 | 3 | 2362.278 | 2362.276 | 0 | 0.9 | 1760 | 362.0 | 362.0 | 362.0 | 7.28 |
| | SLVDKLR.I | | | | | | | | | | | | |
| 834 | R.IIMEQSMR.C | | 504.254 | 2 | 1007.502 | 1007.501 | 0 | 0.2 | 1780 | 486.8 | 486.8 | 486.8 | 7.34 |
| 834 | R.IIMEQSMR.C | | 504.254 | 2 | 1007.502 | 1007.501 | 0 | 0.2 | 1780 | 461.0 | 461.0 | 461.0 | 7.10 |
| 834 | R.IIMEQSMR.C | | 504.254 | 2 | 1007.501 | 1007.501 | 0 | 0.1 | 1780 | 382.8 | 382.8 | 382.8 | 16.72 |
| 834 | R.IIMEQSMR.C | | 504.254 | 2 | 1007.501 | 1007.501 | 0 | −0.1 | 1780 | 379.6 | 379.6 | 379.6 | 6.87 |
| 834 | R.IIMEQSMR.C | | 504.254 | 2 | 1007.501 | 1007.501 | 0 | 0.0 | 1780 | 368.7 | 368.7 | 368.7 | 6.72 |
| 834 | R.IIMEQSMR.C | | 504.254 | 2 | 1007.502 | 1007.501 | 0 | 0.3 | 1780 | 368.6 | 368.6 | 368.6 | 16.72 |
| 1531 | R.IIM[+15.995]E | M[+16] | 512.252 | 2 | 1023.496 | 1023.496 | 0 | 0.1 | 1780 | 366.5 | 366.5 | 4.4 | 6.72 |
| | QSMR.C | | | | | | | | | | | | |
| 834 | R.IIMEQSMR.C | | 504.254 | 2 | 1007.501 | 1007.501 | 0 | 0.1 | 1780 | 334.2 | 334.2 | 334.2 | 5.33 |
| 849 | K.VYSLLFADQLSYE | | 937.489 | 2 | 1873.971 | 1873.969 | 0 | 0.7 | 1891 | 434.3 | 427.9 | 427.9 | 7.83 |
| | VAR.Q | | | | | | | | | | | | |
| 4629 | R.EDYQLVMVC | C[+57] | 743.979 | 8 | 5944.781 | 5943.780 | 1 | −0.4 | 1922 | 308.1 | 308.1 | 25.4 | 2.53 |
| | [+57.021] | *2, | | | | | | | | | | | |
| | DGDWEHC | Y[+80] | | | | | | | | | | | |
| | [+57.021]Y | | | | | | | | | | | | |
| | [+79.966]LP | | | | | | | | | | | | |
| | SAFSQHKVFVTP | | | | | | | | | | | | |
| | QAPLEAIQAYLAGHY | | | | | | | | | | | | |
| | RVPK.Q | | | | | | | | | | | | |
| 851 | K.VFVTPQAPLEAIQ | | 667.871 | 4 | 2668.462 | 2668.461 | 0 | 0.3 | 1948 | 861.5 | 861.5 | 861.5 | 15.65 |
| | AYLAGHYRVPK.Q | | | | | | | | | | | | |
| 1411 | K.VFVTPQAPLEAIQ | | 782.087 | 3 | 2344.245 | 2344.245 | 0 | 0.0 | 1948 | 763.7 | 763.7 | 763.7 | 13.96 |
| | AYLAGHYR.V | | | | | | | | | | | | |
| 851 | K.VFVTPQAPLEAIQ | | 667.871 | 4 | 2668.462 | 2668.461 | 0 | 0.5 | 1948 | 699.3 | 699.3 | 699.3 | 11.77 |
| | AYLAGHYRVPK.Q | | | | | | | | | | | | |
| 851 | K.VFVTPQAPLEAIQ | | 890.159 | 3 | 2668.462 | 2668.461 | 0 | 0.5 | 1948 | 479.5 | 479.5 | 479.5 | 7.28 |
| | AYLAGHYRVPK.Q | | | | | | | | | | | | |
| 959 | K.VFVTPQAPLEAIQ | | 789.226 | 5 | 3942.102 | 3942.103 | 0 | 0.0 | 1948 | 474.8 | 424.3 | 424.3 | 6.64 |
| | AYLAGHYRVPKQTLS | | | | | | | | | | | | |
| | AAAVFNDR.L | | | | | | | | | | | | |
| 814 | K.QTLSAAAVFNDR. | | 646.833 | 2 | 1292.660 | 1292.659 | 0 | 0.1 | 1972 | 681.5 | 603.1 | 603.1 | 11.59 |
| | L | | | | | | | | | | | | |
| 814 | K.QTLSAAAVFNDR. | | 646.834 | 2 | 1292.660 | 1292.659 | 0 | 0.5 | 1972 | 494.8 | 445.2 | 445.2 | 7.81 |
| | L | | | | | | | | | | | | |
| 814 | K.QTLSAAAVFNDR. | | 646.834 | 2 | 1292.660 | 1292.659 | 0 | 0.4 | 1972 | 465.2 | 419.8 | 419.8 | 7.57 |
| | L | | | | | | | | | | | | |
| 814 | K.QTLSAAAVFNDR. | | 646.834 | 2 | 1292.660 | 1292.659 | 0 | 0.5 | 1972 | 389.5 | 354.3 | 354.3 | 5.82 |
| | L | | | | | | | | | | | | |
| 910 | K.MQLNVKNVPLKTI | | 551.999 | 3 | 1653.983 | 1653.983 | 0 | 0.1 | 2011 | 621.1 | 621.1 | 621.1 | 10.46 |
| | R.L | | | | | | | | | | | | |
| 1009 | R.LIDPQVDESR.V | | 586.301 | 2 | 1171.595 | 1171.595 | 0 | −0.2 | 2025 | 549.4 | 388.1 | 388.1 | 8.02 |
| 1244 | R.LIDPQVDESRVLG | | 707.886 | 4 | 2828.522 | 2828.519 | 0 | 1.0 | 2025 | 358.2 | 358.2 | 358.2 | 4.91 |
| | ALLPFLDAQYQK.V | | | | | | | | | | | | |
| 871 | R.VLGALLPFLDAQY | | 559.319 | 3 | 1675.943 | 1675.942 | 0 | 0.6 | 2035 | 567.7 | 567.7 | 567.7 | 9.37 |
| | QK.V | | | | | | | | | | | | |
| 871 | R.VLGALLPFLDAQY | | 838.475 | 2 | 1675.943 | 1675.942 | 0 | 1.0 | 2035 | 492.2 | 492.2 | 492.2 | 8.91 |
| | QK.V | | | | | | | | | | | | |
| 843 | R.TRVPQFSFLDIFP | | 847.967 | 2 | 1694.926 | 1694.927 | 0 | −0.3 | 2116 | 562.4 | 562.4 | 562.4 | 8.34 |
| | K.V | | | | | | | | | | | | |
| 843 | R.TRVPQFSFLDIFP | | 565.647 | 3 | 1694.928 | 1694.927 | 0 | 0.6 | 2116 | 559.5 | 559.5 | 559.5 | 8.20 |
| | K.V | | | | | | | | | | | | |
| 843 | R.TRVPQFSFLDIFP | | 565.647 | 3 | 1694.926 | 1694.927 | 0 | −0.4 | 2116 | 559.0 | 559.0 | 559.0 | 8.23 |
| | K.V | | | | | | | | | | | | |
| 843 | R.TRVPQFSFLDIFP | | 565.647 | 3 | 1694.927 | 1694.927 | 0 | 0.4 | 2116 | 506.6 | 506.6 | 506.6 | 7.99 |
| | K.V | | | | | | | | | | | | |
| 843 | R.TRVPQFSFLDIFP | | 847.966 | 2 | 1694.925 | 1694.927 | 0 | −0.9 | 2116 | 497.5 | 497.5 | 497.5 | 7.28 |
| | K.V | | | | | | | | | | | | |
| 843 | R.TRVPQFSFLDIFP | | 565.647 | 3 | 1694.926 | 1694.927 | 0 | 0.1 | 2116 | 484.3 | 484.3 | 484.3 | 8.72 |
| | K.V | | | | | | | | | | | | |
| 843 | R.TRVPQFSFLDIFP | | 565.647 | 3 | 1694.926 | 1694.927 | 0 | −0.3 | 2116 | 454.2 | 454.2 | 454.2 | 7.75 |
| | K.V | | | | | | | | | | | | |
| 843 | R.TRVPQFSFLDIFP | | 565.647 | 3 | 1694.928 | 1694.927 | 10 | 0.6 | 2116 | 441.9 | 441.9 | 441.9 | 7.75 |
| | K.V | | | | | | | | | | | | |
| 843 | R.TRVPQFSFLDIFP | | 565.647 | 3 | 1694.928 | 1694.927 | 0 | 0.6 | 2116 | 427.1 | 427.1 | 427.1 | 7.40 |
| | K.V | | | | | | | | | | | | |
| 843 | R.TRVPQFSFLDIFP | | 565.647 | 3 | 1694.927 | 1694.927 | 0 | 0.3 | 2116 | 399.2 | 399.2 | 399.2 | 8.25 |
| | K.V | | | | | | | | | | | | |
| 843 | R.TRVPQFSFLDIFP | | 565.647 | 3 | 1694.928 | 1694.927 | 0 | 0.6 | 2116 | 375.4 | 375.4 | 375.4 | 7.28 |
| | K.V | | | | | | | | | | | | |
| 830 | R.VPQFSFLDIFPK. | | 719.393 | 2 | 1437.779 | 1437.778 | 0 | 0.8 | 2118 | 626.8 | 626.8 | 626.8 | 10.59 |
| | V | | | | | | | | | | | | |
| 830 | R.VPQFSFLDIFPK. | | 719.393 | 2 | 1437.778 | 1437.778 | 0 | 0.5 | 2118 | 522.6 | 522.6 | 522.6 | 7.78 |
| | V | | | | | | | | | | | | |
| 830 | R.VPQFSFLDIFPK. | | 479.931 | 3 | 1437.779 | 1437.778 | 0 | 0.7 | 2118 | 481.0 | 481.0 | 481.0 | 7.22 |
| | V | | | | | | | | | | | | |
| 830 | R.VPQFSFLDIFPK. | | 479.931 | 3 | 1437.778 | 1437.778 | 0 | 0.4 | 2118 | 468.3 | 468.3 | 468.3 | 6.73 |
| | V | | | | | | | | | | | | |
| 1740 | R.SDTEPGMDLWEFC | | 1052.468 | 3 | 3155.390 | 3155.387 | 0 | 0.8 | 2148 | 675.9 | 675.9 | 675.9 | 11.58 |
| | [+57.021]SETFQR | | | | | | | | | | | | |
| | PYQYLR.RC[+57] | | | | | | | | | | | | |
| 3308 | R.SDTEPGMDLWEFC | | 829.129 | 4 | 3313.496 | 3311.489 | 2 | 0.1 | 2148 | 420.0 | 420.0 | 420.0 | 6.63 |
| | [+57.021]SETFQR | | | | | | | | | | | | |
| | PYQYLRR.FC[+57] | | | | | | | | | | | | |
| 1773 | R.FLNYQLR.D | | 477.264 | 2 | 953.520 | 953.520 | 0 | −0.1 | 2221 | 449.1 | 449.1 | 449.1 | 6.94 |
| 1773 | R.FLNYQLR.D | | 477.264 | 2 | 953.520 | 953.520 | 0 | 0.5 | 2221 | 378.6 | 270.0 | 270.0 | 4.48 |
| 1773 | R.FLNYQLR.D | | 477.264 | 2 | 953.520 | 953.520 | 0 | −0.1 | 2221 | 350.5 | 239.1 | 239.1 | 5.02 |
| 1088 | K.HMVTMDGVREEDL | | 735.026 | 3 | 2203.063 | 2203.063 | 0 | 0.0 | 2277 | 564.2 | 564.2 | 564.2 | 9.77 |
| | APFSLR.K | | | | | | | | | | | | |
| 1088 | K.HMVTMDGVREEDL | | 1102.033 | 2 | 2203.058 | 2203.063 | 0 | 2.4 | 2277 | 458.5 | 458.5 | 458.5 | 5.66 |
| | APFSLR.K | | | | | | | | | | | | |
| 1883 | K.HMVTMDGVREEDL | | 583.545 | 1 | 2331.158 | 2331.158 | 0 | −0.1 | 2277 | 454.4 | 454.4 | 454.4 | 7.44 |
| | APFSLRK.R | | | | | | | | | | | | |
| 2226 | K.HMVTMDGVR.E | | 523.250 | 2 | 1045.492 | 45.492 | 0 | 0.1 | 2277 | 429.8 | 429.8 | 429.8 | 6.99 |
| 3135 | K.HM[+15.995]VT | | 740.690 | 3 | 2220.055 | 2219.058 | 1 | −3.0 | 2277 | 369.2 | 369.2 | 158.1 | 6.34 |
| | MDGVREEDLAPFSLR | | | | | | | | | | | | |
| | .KM[+16] | | | | | | | | | | | | |
| 1883 | K.HMVTMDGVREEDL | | 777.724 | 3 | 2331.157 | 2331.158 | 0 | −0.7 | 2277 | 353.9 | 353.9 | 353.9 | 3.27 |
| | APFSLRK.R | | | | | | | | | | | | |
| 1121 | R.EEDLAPFSLRK.R | | 435.566 | 3 | 1304.685 | 1304.685 | 0 | 0.1 | 2286 | 613.8 | 613.8 | 613.8 | 10.40 |
| 930 | R.EEDLAPFSLR.K | | 588.798 | 2 | 1176.590 | 1176.590 | 0 | 0.0 | 2286 | 517.5 | 333.7 | 333.7 | 6.53 |
| 930 | R.EEDLAPFSLR.K | | 588.799 | 2 | 1176.590 | 1176.590 | 0 | 0.1 | 2286 | 441.2 | 387.4 | 387.4 | 7.33 |
| 1186 | K.RWESEPHPYVFFN | | 826.732 | 6 | 4955.353 | 4955.364 | 0 | 2.1 | 2297 | 535.5 | 319.1 | 319.1 | 4.65 |
| | DDHTTMTFIGFHLQP | | | | | | | | | | | | |
| | NINGSVDAISHLTGK | | | | | | | | | | | | |
| | .V | | | | | | | | | | | | |
| 840 | R.WESEPHPYVFFND | | 960.658 | 5 | 4799.263 | 4799.262 | 0 | 0.1 | 2298 | 828.7 | 828.7 | 828.7 | 14.16 |
| | DHTTMTFIGFHLQPN | | | | | | | | | | | | |
| | INGSVDAISHLTGK. | | | | | | | | | | | | |
| | V | | | | | | | | | | | | |
| 840 | R.WESEPHPYVFFND | | 960.658 | 5 | 4799.259 | 4799.262 | 0 | −0.8 | 2298 | 659.9 | 470.3 | 470.3 | 9.48 |
| | DHTTMTFIGFHLQPN | | | | | | | | | | | | |
| | INGSVDAISHLTGK. | | | | | | | | | | | | |
| | V | | | | | | | | | | | | |
| 840 | R.WESEPHPYVFFND | | 800.714 | 6 | 4799.249 | 4799.262 | 0 | −2.9 | 2298 | 580.4 | 580.4 | 580.4 | 7.72 |
| | DHTTMTFIGFHLQPN | | | | | | | | | | | | |
| | INGSVDAISHLTGK. | | | | | | | | | | | | |
| | V | | | | | | | | | | | | |
| 1513 | R.WESEPHPYVFFND | | 800.882 | 6 | 4800.256 | 4800.246 | 0 | 1.9 | 2298 | 572.3 | 520.4 | 45.7 | 8.19 |
| | DHTTMTFIGFHLQPN | | | | | | | | | | | | |
| | IN[+0.984]GSVDA | | | | | | | | | | | | |
| | ISHLTGK.VN[+1] | | | | | | | | | | | | |
| 840 | R.WESEPHPYVFFND | | 800.885 | 6 | 4800.273 | 4799.262 | 1 | 1.5 | 2298 | 546.2 | 463.9 | 15.1 | 6.96 |
| | DHTTMTFIGFHLQPN | | | | | | | | | | | | |
| | INGSVDAISHLTGK. | | | | | | | | | | | | |
| | V | | | | | | | | | | | | |
| 840 | R.WESEPHPYVFFND | | 800.717 | 6 | 4799.265 | 4799.262 | 0 | 0.6 | 2298 | 462.7 | 462.7 | 462.7 | 6.89 |
| | DHTTMTFIGFHLQPN | | | | | | | | | | | | |
| | INGSVDAISHLTGK. | | | | | | | | | | | | |
| | V | | | | | | | | | | | | |
| 868 | R.DVMTRDLYQGLLL | | 607.661 | 3 | 1820.969 | 1820.969 | 0 | 0.0 | 2344 | 494.1 | 494.1 | 494.1 | 8.72 |
| | QR.V | | | | | | | | | | | | |
| 868 | R.DVMTRDLYQGLLL | | 607.661 | 3 | 1820.969 | 1820.969 | 0 | 0.0 | 2344 | 303.9 | 303.9 | 303.9 | 5.58 |
| | QR.V | | | | | | | | | | | | |
| 1068 | R.DLYQGLLLQRVPF | | 662.366 | 4 | 2646.442 | 2646.440 | 0 | 0.6 | 2349 | 652.5 | 652.5 | 1652.5 | 11.46 |
| | NVDFDKLPR.H | | | | | | | | | | | | |
| 1068 | R.DLYQGLLLQRVPF | | 662.366 | 4 | 2646.442 | 2646.440 | 10 | 0.5 | 2349 | 600.3 | 600.3 | 600.3 | 10.46 |
| | NVDFDKLPR.H | | | | | | | | | | | | |
| 1068 | R.DLYQGLLLQRVPF | | 882.819 | 3 | 2646.442 | 2646.440 | 0 | 0.8 | 2349 | 582.8 | 582.8 | 582.8 | 9.46 |
| | NVDFDKLPR.H | | | | | | | | | | | | |
| 984 | R.DLYQGLLLQR.V | | 609.846 | 2 | 1218.684 | 1218.684 | 0 | −0.3 | 2349 | 582.0 | 582.0 | 582.0 | 8.86 |
| 1068 | R.DLYQGLLLQRVPF | | 882.820 | 3 | 2646.445 | 2646.440 | 0 | 1.6 | 2349 | 568.7 | 568.7 | 568.7 | 8.73 |
| | NVDFDKLPR.H | | | | | | | | | | | | |
| 1068 | R.DLYQGLLLQRVPF | | 662.366 | 4 | 2646.441 | 2646.440 | 0 | 0.4 | 2349 | 566.1 | 566.1 | 566.1 | 9.46 |
| | NVDFDKLPR.H | | | | | | | | | | | | |
| 1068 | R.DLYQGLLLQRVPF | | 530.094 | 5 | 2646.440 | 2646.440 | 0 | −0.3 | 2349 | 548.7 | 548.7 | 548.7 | 8.03 |
| | NVDFDKLPR.H | | | | | | | | | | | | |
| 984 | R.DLYQGLLLQR.V | | 406.900 | 3 | 1218.684 | 1218.684 | 0 | 0.0 | 2349 | 537.6 | 537.6 | 537.6 | 8.16 |
| 984 | R.DLYQGLLLQR.V | | 609.846 | 2 | 1218.684 | 1218.684 | 0 | −0.2 | 2349 | 459.6 | 459.6 | 459.6 | 7.33 |
| 1068 | R.DLYQGLLLQRVPF | | 882.818 | 3 | 2646.441 | 2646.440 | 0 | 0.2 | 2349 | 448.1 | 448.1 | 448.1 | 6.97 |
| | NVDFDKLPR.H | | | | | | | | | | | | |
| 841 | R.VPFNVDFDKLPR. | | 482.930 | 3 | 1446.774 | 1446.774 | 0 | 0.0 | 2359 | 651.5 | 651.5 | 651.5 | 11.40 |
| | H | | | | | | | | | | | | |
| 841 | R.VPFNVDFDKLPR. | | 482.929 | 3 | 1446.774 | 1446.774 | 0 | −0.1 | 2359 | 627.5 | 627.5 | 627.5 | 10.40 |
| | H | | | | | | | | | | | | |
| 841 | R.VPFNVDFDKLPR. | | 723.891 | 2 | 1446.774 | 1446.774 | 0 | 0.1 | 2359 | 543.7 | 543.7 | 543.7 | 8.56 |
| | H | | | | | | | | | | | | |
| 841 | R.VPFNVDFDKLPR. | | 482.930 | 3 | 1446.774 | 1446.774 | 0 | 0.2 | 2359 | 486.7 | 486.7 | 486.7 | 18.35 |
| | H | | | | | | | | | | | | |
| 841 | R.VPFNVDFDKLPR. | | 482.930 | 3 | 1446.774 | 1446.774 | 0 | −0.1 | 2359 | 321.8 | 321.8 | 321.8 | 5.29 |
| | H | | | | | | | | | | | | |
| 841 | R.VPFNVDFDKLPR. | | 723.891 | 2 | 1446.774 | 1446.774 | 0 | 0.1 | 2359 | 304.5 | 304.5 | 304.5 | 5.21 |
| | H | | | | | | | | | | | | |
| 1748 | R.LC[+57.021]LT | C[+57] | 984.496 | 3 | 2951.475 | 2951.474 | 0 | 0.2 | 2377 | 543.6 | 543.6 | 543.6 | 18.55 |
| | LGIPQATDPDKTYEL | | | | | | | | | | | | |
| | TTDNMLK.I | | | | | | | | | | | | |
| 1798 | K.ILAIEMR.F | | 423.249 | 2 | 845.491 | 845.491 | 0 | 0.0 | 2403 | 458.5 | 334.3 | 334.3 | 5.81 |
| 1798 | K.ILAIEMR.F | | 423.249 | 2 | 845.491 | 845.491 | 0 | 0.0 | 2403 | 395.4 | 395.4 | 395.4 | 6.72 |
| 1798 | K.ILAIEMR.F | | 845.491 | 1 | 845.491 | 845.491 | 0 | −0.7 | 2403 | 387.6 | 387.6 | 387.6 | 6.01 |
| 3975 | R.LIKFLSDLRR.G | | 420.931 | 3 | 1260.778 | 1260.779 | 0 | −0.2 | 2429 | 435.5 | 435.5 | 435.5 | 6.96 |
| 3975 | R.LIKFLSDLRR.G | | 420.931 | 3 | 1260.779 | 1260.779 | 0 | 0.1 | 2429 | 380.8 | 380.8 | 380.8 | 6.37 |
| 1781 | K.FLSDLRR.G | | 453.761 | 2 | 906.515 | 906.516 | 0 | −0.3 | 2432 | 330.7 | 227.7 | 227.7 | 4.75 |
| 1781 | K.FLSDLRR.G | | 453.761 | 2 | 906.516 | 906.516 | 0 | 0.1 | 2432 | 324.3 | 177.1 | 177.1 | 4.42 |
| 1781 | K.FLSDLRR.G | | 453.761 | 2 | 906.516 | 906.516 | 0 | 0.0 | 2432 | 316.5 | 164.2 | 164.2 | 4.48 |
| 1506 | R.GGTN[+0.984]A | N[+1] | 406.563 | 3 | 1217.674 | 1217.674 | 0 | 0.4 | 2439 | 504.8 | 464.5 | 464.5 | 7.62 |
| | DTIKLVK.V | | | | | | | | | | | | |
| 866 | K.VHGGTTADMIYSR | | 704.338 | 2 | 1407.668 | 1407.669 | 0 | 0.3 | 2451 | 752.8 | 752.8 | 752.8 | 12.88 |
| | .V | | | | | | | | | | | | |
| 1538 | K.VHGGTTADM | M[+16] | 712.336 | 2 | 1423.665 | 1423.663 | 0 | 0.9 | 2451 | 597.7 | 597.7 | 597.7 | 9.59 |
| | [+15.995] | | | | | | | | | | | | |
| | IYSR.V | | | | | | | | | | | | |
| 1538 | K.VHGGTTADM | M[+16] | 475.226 | 3 | 1423.664 | 1423.663 | 0 | 0.2 | 2451 | 518.7 | 518.7 | 518.7 | 7.81 |
| | [+15.995] | | | | | | | | | | | | |
| | IYSR.V | | | | | | | | | | | | |
| 866 | K.VHGGTTADMIYSR | | 469.894 | 3 | 1407.669 | 1407.669 | 0 | 0.0 | 2451 | 516.0 | 516.0 | 516.0 | 7.81 |
| | .V | | | | | | | | | | | | |
| 1538 | K.VHGGTTADM | M[+16] | 475.226 | 3 | 1423.663 | 1423.663 | 0 | 0.0 | 2451 | 500.1 | 500.1 | 500.1 | 7.81 |
| | [+15.995] | | | | | | | | | | | | |
| | IYSR.V | | | | | | | | | | | | |
| 866 | K.VHGGTTADMIYSR | | 469.894 | 3 | 1407.669 | 1407.669 | 0 | 0.0 | 2451 | 477.2 | 477.2 | 477.2 | 7.57 |
| | .V | | | | | | | | | | | | |
| 1538 | K.VHGGTTADM | M[+16] | 475.226 | 3 | 1423.663 | 1423.663 | 0 | 0.0 | 2451 | 476.2 | 476.2 | 476.2 | 7.57 |
| | [+15.995] | | | | | | | | | | | | |
| | IYSR.V | | | | | | | | | | | | |
| 1538 | K.VHGGTTADM | M[+16] | 475.226 | 3 | 1423.664 | 1423.663 | 0 | 0.4 | 2451 | 473.9 | 473.9 | 473.9 | 7.57 |
| | [+15.995] | | | | | | | | | | | | |
| | IYSR.V | | | | | | | | | | | | |
| 866 | K.VHGGTTADMIYSR | | 469.894 | 3 | 1407.668 | 1407.669 | 0 | 0.2 | 2451 | 473.5 | 1473.5 | 473.5 | 8.30 |
| | .V | | | | | | | | | | | | |
| 866 | K.VHGGTTADMIYSR | | 469.894 | 3 | 1407.668 | 1407.669 | 0 | −0.3 | 2451 | 448.8 | 448.8 | 448.8 | 6.86 |
| | .V | | | | | | | | | | | | |
| 866 | K.VHGGTTADMIYSR | | 704.338 | 2 | 1407.668 | 1407.669 | 0 | −0.2 | 2451 | 436.2 | 436.2 | 436.2 | 8.19 |
| | .V | | | | | | | | | | | | |
| 866 | K.VHGGTTADMIYSR | | 469.894 | 3 | 1407.668 | 1407.669 | 0 | 0.1 | 2451 | 416.6 | 416.6 | 416.6 | 7.22 |
| | .V | | | | | | | | | | | | |
| 866 | K.VHGGTTADMIYSR | | 469.894 | 3 | 1407.667 | 1407.669 | 0 | 0.8 | 2451 | 375.5 | 375.5 | 375.5 | 6.16 |
| | .V | | | | | | | | | | | | |
| 866 | K.VHGGTTADMIYSR | | 469.894 | 3 | 1407.668 | 1407.669 | 0 | −0.2 | 2451 | 365.0 | 365.0 | 365.0 | 7.10 |
| | .V | | | | | | | | | | | | |
| 866 | K.VHGGTTADMIYSR | | 704.334 | 2 | 1407.661 | 1407.669 | 0 | −5.7 | 2451 | 356.1 | 356.1 | 356.1 | 4.01 |
| | .V | | | | | | | | | | | | |
| 866 | K.VHGGTTADMIYSR | | 469.894 | 3 | 1407.668 | 1407.669 | 0 | −0.2 | 2451 | 326.0 | 326.0 | 326.0 | 15.48 |
| | .V | | | | | | | | | | | | |
| 866 | K.VHGGTTADMIYSR | | 469.894 | 3 | 1407.668 | 1407.669 | 0 | −0.3 | 2451 | 316.0 | 316.0 | 316.0 | 4.28 |
| | .V | | | | | | | | | | | | |
| 866 | K.VHGGTTADMIYSR | | 704.338 | 2 | 1407.668 | 1407.669 | 0 | −0.1 | 2451 | 304.8 | 304.8 | 304.8 | 15.63 |
| | .V | | | | | | | | | | | | |
| 1926 | R.VREAENVAFANK. | | 449.906 | 3 | 1347.702 | 1347.702 | 0 | 0.2 | 2464 | 414.4 | 414.1 | 414.1 | 6.80 |
| | D | | | | | | | | | | | | |
| 1926 | R.VREAENVAFANK. | | 674.354 | 2 | 1347.701 | 1347.702 | 0 | 0.4 | 2464 | 354.4 | 354.4 | 354.4 | 4.43 |
| | D | | | | | | | | | | | | |
| 1926 | R.VREAENVAFANK. | | 449.905 | 3 | 1347.702 | 1347.702 | 0 | −0.1 | 2464 | 324.5 | 324.5 | 324.5 | 15.29 |
| | D | | | | | | | | | | | | |
| 3318 | K.HSEEMIC | C[+57] | 669.985 | 3 | 2007.939 | 2007.938 | 0 | 0.7 | 2530 | 394.3 | 394.3 | 394.3 | 17.28 |
| | [+57.021]R | | | | | | | | | | | | |
| | LESAGLGYR.V | | | | | | | | | | | | |
| 890 | R.LESAGLGYR.V | | 483.256 | 2 | 965.505 | 965.505 | 0 | 0.1 | 2538 | 588.5 | 588.5 | 588.5 | 8.62 |
| 890 | R.LESAGLGYR.V | | 483.256 | 2 | 965.505 | 965.505 | 0 | 0.1 | 2538 | 548.4 | 548.4 | 548.4 | 7.78 |
| 890 | R.LESAGLGYR.V | | 483.256 | 2 | 965.505 | 965.505 | 0 | −0.4 | 2538 | 521.3 | 521.3 | 521.3 | 7.07 |
| 890 | R.LESAGLGYR.V | | 483.256 | 2 | 965.505 | 965.505 | 0 | −0.1 | 2538 | 509.9 | 509.9 | 509.9 | 7.81 |
| 890 | R.LESAGLGYR.V | | 483.256 | 2 | 965.505 | 965.505 | 0 | −0.3 | 2538 | 474.1 | 380.9 | 380.9 | 7.10 |
| 890 | R.LESAGLGYR.V | | 483.256 | 2 | 965.505 | 965.505 | 0 | 0.3 | 2538 | 389.1 | 389.1 | 389.1 | 6.72 |
| 890 | R.LESAGLGYR.V | | 483.256 | 2 | 965.505 | 965.505 | 0 | 0.0 | 2538 | 330.3 | 330.3 | 330.3 | 5.33 |
| 1190 | R.VSMEETADRLG | | 591.977 | 3 | 1773.917 | 1773.916 | 0 | 0.2 | 2547 | 634.2 | 634.2 | 634.2 | 10.77 |
| | SIPLR.Q | | | | | | | | | | | | |
| 1587 | R.VSM[+15.995]E | M[+16] | 597.308 | 3 | 1789.910 | 1789.911 | 0 | −0.5 | 2547 | 476.8 | 476.8 | 476.8 | 6.80 |
| | ETADRLGSIPLR.Q | | | | | | | | | | | | |
| 1587 | R.VSM[+15.995]E | M[+16] | 597.308 | 3 | 1789.910 | 1789.911 | 0 | −0.5 | 2547 | 475.4 | 475.4 | 475.4 | 16.80 |
| | ETADRLGSIPLR.Q | | | | | | | | | | | | |
| 1190 | R.VSMEETADRLGSI | | 591.977 | 3 | 1773.917 | 1773.916 | 0 | 0.2 | 2547 | 449.0 | 449.0 | 449.0 | 7.75 |
| | PLR.Q | | | | | | | | | | | | |
| 1587 | R.VSM[+15.995]E | M[+16] | 597.309 | 3 | 1789.912 | 1789.911 | 0 | 0.3 | 2547 | 440.6 | 440.6 | 440.6 | 7.28 |
| | ETADRLGSIPLR.Q | | | | | | | | | | | | |
| 1190 | R.VSMEETADRLGSI | | 591.977 | 3 | 1773.917 | 1773.916 | 0 | 0.2 | 2547 | 420.9 | 420.9 | 420.9 | 7.64 |
| | PLR.Q | | | | | | | | | | | | |
| 1190 | R.VSMEETADRLGSI | | 591.977 | 3 | 1773.916 | 1773.916 | 0 | 0.0 | 2547 | 416.2 | 416.2 | 416.2 | 7.64 |
| | PLR.Q | | | | | | | | | | | | |
| 1190 | R.VSMEETADRLGSI | | 887.462 | 2 | 1773.916 | 1773.916 | 0 | 0.0 | 2547 | 314.2 | 314.2 | 314.2 | 15.42 |
| | PLR.Q | | | | | | | | | | | | |
| 1587 | R.VSM[+15.995]E | M[+16] | 895.458 | 2 | 1789.910 | 1789.911 | 0 | −1.0 | 2547 | 306.8 | 306.8 | 306.8 | 14.87 |
| | ETADRLGSIPLR.Q | | | | | | | | | | | | |
| 1234 | R.VHALPPSLIPLVW | | 944.026 | 4 | 3773.082 | 3773.079 | 0 | 0.9 | 2568 | 761.7 | 761.7 | 761.7 | 13.39 |
| | DFGQLSDVAEKLYIQ | | | | | | | | | | | | |
| | QIVQR.L | | | | | | | | | | | | |
| 1139 | R.VHALPPSLIPLVW | | 877.811 | 3 | 2631.420 | 2631.418 | 0 | 0.6 | 2568 | 697.7 | 697.7 | 697.7 | 11.96 |
| | DFGQLSDVAEK.L | | | | | | | | | | | | |
| 1139 | R.VHALPPSLIPLVW | | 877.812 | 3 | 2631.420 | 2631.418 | 0 | 0.8 | 2568 | 624.9 | 624.9 | 624.9 | 10.96 |
| | DFGQLSDVAEK.L | | | | | | | | | | | | |
| 1139 | R.VHALPPSLIPLVW | | 877.813 | 3 | 2631.423 | 2631.418 | 0 | 1.9 | 2568 | 594.1 | 594.1 | 594.1 | 9.96 |
| | DFGQLSDVAEK.L | | | | | | | | | | | | |
| 1234 | R.VHALPPSLIPLVW | | 944.025 | 4 | 3773.078 | 3773.079 | 0 | 0.2 | 2568 | 592.0 | 592.0 | 592.0 | 8.66 |
| | DFGQLSDVAEKLYIQ | | | | | | | | | | | | |
| | QIVQR.L | | | | | | | | | | | | |
| 981 | K.LYIQQIVQR.L | | 580.843 | 2 | 1160.678 | 1160.679 | 0 | −0.2 | 2592 | 352.0 | 352.0 | 352.0 | 5.48 |
| 2006 | R.LVESISLDENGTR | | 716.867 | 2 | 1432.726 | 1432.728 | 0 | −1.0 | 2601 | 624.8 | 586.7 | 1586.7 | 9.88 |
| | .V | | | | | | | | | | | | |
| 2006 | R.LVESISLDENGTR | | 478.248 | 3 | 1432.728 | 1432.728 | 0 | 0.2 | 2601 | 467.4 | 467.4 | 467.4 | 6.73 |
| | .V | | | | | | | | | | | | |
| 938 | R.FFPKPYDDSR.L | | 636.307 | 2 | 1271.606 | 1271.606 | 0 | 0.2 | 2710 | 525.0 | 525.0 | 525.0 | 7.78 |
| 883 | R.FFPKPYDDSRLLL | | 742.392 | 3 | 2225.161 | 2225.160 | 0 | 0.4 | 2710 | 475.6 | 475.6 | 475.6 | 8.48 |
| | DEITR.A | | | | | | | | | | | | |
| 883 | R.FFPKPYDDSRLLL | | 557.046 | 4 | 2225.160 | 2225.160 | 0 | 0.0 | 2710 | 378.5 | 378.5 | 378.5 | 7.53 |
| | DEITR.A | | | | | | | | | | | | |
| 938 | R.FFPKPYDDSR.L | | 424.540 | 3 | 1271.606 | 1271.606 | 0 | 0.5 | 2710 | 375.5 | 375.5 | 375.5 | 6.87 |
| 938 | R.FFPKPYDDSR.L | | 424.540 | 3 | 1271.606 | 1271.606 | 0 | 0.4 | 2710 | 374.4 | 374.4 | 374.4 | 6.72 |
| 938 | R.FFPKPYDDSR.L | | 424.540 | 3 | 1271.606 | 1271.606 | 0 | 0.1 | 2710 | 355.8 | 355.8 | 355.8 | 5.33 |
| 938 | R.FFPKPYDDSR.L | | 424.540 | 3 | 1271.606 | 1271.606 | 0 | 0.3 | 2710 | 338.4 | 338.4 | 338.4 | 5.33 |
| 938 | R.FFPKPYDDSR.L | | 424.540 | 3 | 1271.606 | 1271.606 | 0 | 0.2 | 2710 | 336.2 | 336.2 | 336.2 | 5.33 |
| 902 | R.LLLDEITRAQDLF | | 809.133 | 3 | 2425.383 | 2425.381 | 0 | 0.6 | 2720 | 669.6 | 669.6 | 669.6 | 11.46 |
| | LDGVPLRK.T | | | | | | | | | | | | |
| 1256 | R.LLLDEITRAQDLF | | 766.435 | 3 | 2297.290 | 2297.286 | 0 | 1.7 | 2720 | 644.8 | 644.8 | 644.8 | 10.77 |
| | LDGVPLR.K | | | | | | | | | | | | |
| 902 | R.LLLDEITRAQDLF | | 809.132 | 3 | 2425.383 | 2425.381 | 0 | 0.5 | 2720 | 595.3 | 595.3 | 595.3 | 9.46 |
| | LDGVPLRK.T | | | | | | | | | | | | |
| 902 | R.LLLDEITRAQDLF | | 607.101 | 4 | 2425.381 | 2425.381 | 0 | −0.2 | 2720 | 513.5 | 513.5 | 513.5 | 8.41 |
| | LDGVPLRK.T | | | | | | | | | | | | |
| 2028 | R.LLLDEITR.A | | 486.790 | 2 | 972.572 | 972.572 | 0 | 0.0 | 2720 | 476.6 | 476.6 | 476.6 | 7.33 |
| 902 | R.LLLDEITRAQDLF | | 809.132 | 3 | 2425.381 | 2425.381 | 0 | −0.3 | 2720 | 463.5 | 463.5 | 463.5 | 6.26 |
| | LDGVPLRK.T | | | | | | | | | | | | |
| 2028 | R.LLLDEITR.A | | 486.790 | 2 | 972.572 | 972.572 | 0 | −0.2 | 2720 | 428.1 | 428.1 | 428.1 | 6.99 |
| 902 | R.LLLDEITRAQDLF | | 485.882 | 5 | 2425.379 | 2425.381 | 0 | 0.9 | 2720 | 414.4 | 414.4 | 414.4 | 6.75 |
| | LDGVPLRK.T | | | | | | | | | | | | |
| 2028 | R.LLLDEITR.A | | 486.790 | 2 | 972.573 | 972.572 | 0 | 0.1 | 2720 | 406.8 | 406.8 | 406.8 | 7.22 |
| 902 | R.LLLDEITRAQDLF | | 607.101 | 4 | 2425.383 | 2425.381 | 0 | 0.5 | 2720 | 391.7 | 391.7 | 391.7 | 7.21 |
| | LDGVPLRK.T | | | | | | | | | | | | |
| 1256 | R.LLLDEITRAQDLF | | 575.077 | 4 | 2297.287 | 2297.286 | 0 | 0.1 | 2720 | 388.1 | 388.1 | 388.1 | 6.93 |
| | LDGVPLR.K | | | | | | | | | | | | |
| 902 | R.LLLDEITRAQDLF | | 809.132 | 3 | 2425.382 | 2425.381 | 0 | 0.2 | 2720 | 378.2 | 378.2 | 378.2 | 6.97 |
| | LDGVPLRK.T | | | | | | | | | | | | |
| 2028 | R.LLLDEITR.A | | 486.790 | 2 | 972.572 | 972.572 | 0 | −0.1 | 2720 | 364.4 | 364.4 | 364.4 | 6.72 |
| 1256 | R.LLLDEITRAQDLF | | 1149.149 | 2 | 2297.290 | 2297.286 | 0 | 1.6 | 2720 | 307.9 | 307.9 | 307.9 | 5.81 |
| | LDGVPLR.K | | | | | | | | | | | | |
| 922 | R.AQDLFLDGVPLRK | | 736.417 | 2 | 1471.827 | 1471.827 | 0 | 0.3 | 2728 | 665.6 | 665.6 | 665.6 | 11.40 |
| | .T | | | | | | | | | | | | |
| 1189 | R.AQDLFLDGVPLR. | | 672.370 | 2 | 1343.732 | 1343.732 | 0 | 0.2 | 2728 | 616.9 | 616.9 | 616.9 | 10.59 |
| | K | | | | | | | | | | | | |
| 922 | R.AQDLFLDGVPLRK | | 491.280 | 3 | 1471.827 | 1471.827 | 0 | 0.0 | 2728 | 606.4 | 606.4 | 606.4 | 10.40 |
| | .T | | | | | | | | | | | | |
| 922 | R.AQDLFLDGVPLRK | | 491.280 | 3 | 1471.827 | 1471.827 | 0 | 0.0 | 2728 | 587.9 | 587.9 | 587.9 | 9.40 |
| | .T | | | | | | | | | | | | |
| 922 | R.AQDLFLDGVPLRK | | 491.281 | 3 | 1471.827 | 1471.827 | 0 | 0.1 | 2728 | 553.3 | 553.3 | 1553.3 | 8.56 |
| | .T | | | | | | | | | | | | |
| 922 | R.AQDLFLDGVPLRK | | 491.281 | 3 | 1471.827 | 1471.827 | 0 | 0.1 | 2728 | 476.2 | 476.2 | 476.2 | 8.11 |
| | .T | | | | | | | | | | | | |
| 922 | R.AQDLFLDGVPLRK | | 491.280 | 3 | 1471.827 | 1471.827 | 0 | 0.1 | 2728 | 442.2 | 442.2 | 442.2 | 7.13 |
| | .T | | | | | | | | | | | | |
| 922 | R.AQDLFLDGVPLRK | | 491.280 | 3 | 1471.826 | 1471.827 | 0 | −0.4 | 2728 | 377.2 | 331.2 | 331.2 | 4.92 |
| | .T | | | | | | | | | | | | |
| 922 | R.AQDLFLDGVPLRK | | 491.280 | 3 | 1471.827 | 1471.827 | 0 | 0.0 | 2728 | 371.0 | 371.0 | 371.0 | 6.91 |
| | .T | | | | | | | | | | | | |
| 922 | R.AQDLFLDGVPLRK | | 491.281 | 3 | 1471.827 | 1471.827 | 0 | 0.1 | 2728 | 347.7 | 347.7 | 347.7 | 5.14 |
| | .T | | | | | | | | | | | | |
| 2639 | K.IPLFLVGKPGSSK | | 448.275 | 3 | 1342.809 | 1342.809 | 0 | −0.2 | 2763 | 344.2 | 344.2 | 344.2 | 5.48 |
| | .S | | | | | | | | | | | | |
| 850 | K.TIVADAMQGPAAY | | 963.475 | 2 | 1925.943 | 1925.943 | 0 | 0.1 | 2780 | 664.0 | 664.0 | 664.0 | 11.96 |
| | SDLFR.S | | | | | | | | | | | | |
| 850 | K.TIVADAMQGPAAY | | 963.475 | 2 | 1925.943 | 1925.943 | 0 | 0.0 | 2780 | 654.2 | 654.2 | 654.2 | 11.96 |
| | SDLFR.S | | | | | | | | | | | | |
| 850 | K.TIVADAMQGPAAY | | 963.476 | 2 | 1925.945 | 1925.943 | 0 | 1.4 | 2780 | 650.1 | 650.1 | 650.1 | 11.96 |
| | SDLFR.S | | | | | | | | | | | | |
| 850 | K.TIVADAMQGPAAY | | 642.653 | 3 | 1925.944 | 1925.943 | 0 | 0.9 | 2780 | 646.0 | 646.0 | 646.0 | 10.37 |
| | SDLFR.S | | | | | | | | | | | | |
| 850 | K.TIVADAMQGPAAY | | 963.475 | 2 | 1925.942 | 1925.943 | 0 | −0.1 | 2780 | 586.5 | 586.5 | 586.5 | 9.96 |
| | SDLFR.S | | | | | | | | | | | | |
| 850 | K.TIVADAMQGPAAY | | 642.654 | 3 | 1925.946 | 1925.943 | 0 | 1.9 | 2780 | 581.1 | 581.1 | 581.1 | 9.37 |
| | SDLFR.S | | | | | | | | | | | | |
| 850 | K.TIVADAMQGPAAY | | 642.653 | 3 | 1925.943 | 1925.943 | 0 | 0.3 | 2780 | 575.7 | 575.7 | 575.7 | 9.37 |
| | SDLFR.S | | | | | | | | | | | | |
| 1545 | K.TIVADAM | M[+16] | 971.472 | 2 | 1941.937 | 1941.938 | 0 | 0.1 | 2780 | 574.4 | 574.4 | 574.4 | 9.96 |
| | [+15.995] | | | | | | | | | | | | |
| | QGPAAYSDLFR. | | | | | | | | | | | | |
| | S | | | | | | | | | | | | |
| 850 | K.TIVADAMQGPAAY | | 963.475 | 2 | 1925.943 | 1925.943 | 0 | 0.1 | 2780 | 551.3 | 517.9 | 517.9 | 9.12 |
| | SDLFR.S | | | | | | | | | | | | |
| 850 | K.TIVADAMQGPAAY | | 963.474 | 2 | 1925.941 | 1925.943 | 0 | −0.6 | 2780 | 482.5 | 482.5 | 482.5 | 7.48 |
| | SDLFR.S | | | | | | | | | | | | |
| 850 | K.TIVADAMQGPAAY | | 642.653 | 3 | 1925.944 | 1925.943 | 0 | 0.9 | 2780 | 405.2 | 405.2 | 405.2 | 7.24 |
| | SDLFR.S | | | | | | | | | | | | |
| 850 | K.TIVADAMQGPAAY | | 642.652 | 3 | 1925.942 | 1925.943 | 0 | −0.2 | 2780 | 400.4 | 400.4 | 400.4 | 6.99 |
| | SDLFR.S | | | | | | | | | | | | |
| 850 | K.TIVADAMQGPAAY | | 963.475 | 2 | 1925.942 | 1925.943 | 0 | 0.2 | 2780 | 349.6 | 349.6 | 349.6 | 6.09 |
| | SDLFR.S | | | | | | | | | | | | |
| 1688 | R.SLKQVHLVSFQC | C[+57] | 739.388 | 4 | 2954.530 | 2954.531 | 0 | −0.2 | 2798 | 852.8 | 852.8 | 852.8 | 15.35 |
| | [+57.021] | | | | | | | | | | | | |
| | SPHSTPQ | | | | | | | | | | | | |
| | GIISTFR.Q | | | | | | | | | | | | |
| 1688 | R.SLKQVHLVSFQC | C[+57] | 739.388 | 4 | 2954.530 | 2954.531 | 0 | −0.2 | 2798 | 633.8 | 633.8 | 1633.8 | 10.39 |
| | [+57.021] | | | | | | | | | | | | |
| | SPHSTPQ | | | | | | | | | | | | |
| | GIISTFR.Q | | | | | | | | | | | | |
| 1688 | R.SLKQVHLVSFQC | C[+57] | 591.913 | 5 | 2955.535 | 2954.531 | 1 | 0.3 | 2798 | 441.8 | 441.8 | 441.8 | 7.36 |
| | [+57.021] | | | | | | | | | | | | |
| | SPHSTPQ | | | | | | | | | | | | |
| | GIISTFR.Q | | | | | | | | | | | | |
| 1688 | R.SLKQVHLVSFQC | C[+57] | 591.712 | 5 | 2954.529 | 2954.531 | 0 | −0.7 | 2798 | 325.8 | 325.8 | 325.8 | 4.57 |
| | [+57.021] | | | | | | | | | | | | |
| | SPHSTPQ | | | | | | | | | | | | |
| | GIISTFR.Q | | | | | | | | | | | | |
| 1700 | K.QVHLVSFQC | C[+57] | 657.335 | 4 | 2626.320 | 2626.320 | 0 | 0.1 | 2801 | 372.4 | 372.4 | 372.4 | 7.24 |
| | [+57.021] | | | | | | | | | | | | |
| | SPHSTPQGII | | | | | | | | | | | | |
| | STFR.Q | | | | | | | | | | | | |
| 1416 | R.FQQGKDLQQYVSV | | 1154.274 | 3 | 3460.808 | 3460.803 | 0 | 1.3 | 2828 | 801.2 | 801.2 | 801.2 | 14.07 |
| | VVLDEVGLAEDSPKM | | | | | | | | | | | | |
| | PLK.T | | | | | | | | | | | | |
| 1416 | R.FQQGKDLQQYVSV | | 865.957 | 4 | 3460.805 | 3460.803 | 0 | 0.5 | 2828 | 799.4 | 799.4 | 799.4 | 13.08 |
| | VVLDEVGLAEDSPKM | | | | | | | | | | | | |
| | PLK.T | | | | | | | | | | | | |
| 1416 | R.FQQGKDLQQYVSV | | 865.957 | 4 | 3460.806 | 3460.803 | 0 | 0.7 | 2828 | 792.6 | 792.6 | 792.6 | 13.08 |
| | VVLDEVGLAEDSPKM | | | | | | | | | | | | |
| | PLK.T | | | | | | | | | | | | |
| 961 | R.FQQGKDLQQYVSV | | 1496.269 | 2 | 2991.531 | 2991.531 | 0 | −0.1 | 2828 | 630.4 | 630.4 | 630.4 | 10.39 |
| | VVLDEVGLAEDSPK. | | | | | | | | | | | | |
| | M | | | | | | | | | | | | |
| 1617 | R.FQQGKDLQQYVSV | M[+16] | 869.955 | 4 | 3476.798 | 3476.798 | 0 | −0.1 | 2828 | 616.9 | 616.9 | 616.9 | 10.08 |
| | VVLDEVGLAEDSPKM | | | | | | | | | | | | |
| | [+15.995]PLK.T | | | | | | | | | | | | |
| 961 | R.FQQGKDLQQYVSV | | 997.850 | 3 | 2991.537 | 2991.531 | 0 | 1.9 | 2828 | 554.6 | 554.6 | 554.6 | 7.58 |
| | VVLDEVGLAEDSPK. | | | | | | | | | | | | |
| | M | | | | | | | | | | | | |
| 1617 | R.FQQGKDLQQYVSV | M[+16] | 869.955 | 4 | 3476.799 | 3476.798 | 0 | 0.2 | 2828 | 541.8 | 448.9 | 448.9 | 7.27 |
| | VVLDEVGLAEDSPKM | | | | | | | | | | | | |
| | [+15.995]PLK.T | | | | | | | | | | | | |
| 1617 | R.FQQGKDLQQYVSV | M[+16] | 869.955 | 4 | 3476.798 | 3476.798 | 0 | 0.1 | 2828 | 534.0 | 534.0 | 534.0 | 7.27 |
| | VVLDEVGLAEDSPKM | | | | | | | | | | | | |
| | [+15.995]PLK.T | | | | | | | | | | | | |
| 961 | R.FQQGKDLQQYVSV | | 997.850 | 3 | 2991.535 | 2991.531 | 0 | 1.2 | 2828 | 477.4 | 477.4 | 477.4 | 7.36 |
| | VVLDEVGLAEDSPK. | | | | | | | | | | | | |
| | M | | | | | | | | | | | | |
| 961 | R.FQQGKDLQQYVSV | | 748.639 | 4 | 2991.533 | 2991.531 | 0 | 0.6 | 2828 | 404.5 | 404.5 | 404.5 | 6.42 |
| | VVLDEVGLAEDSPK. | | | | | | | | | | | | |
| | M | | | | | | | | | | | | |
| 961 | R.FQQGKDLQQYVSV | | 748.639 | 4 | 2991.533 | 2991.531 | 0 | 0.7 | 2828 | 379.5 | 379.5 | 379.5 | 6.07 |
| | VVLDEVGLAEDSPK. | | | | | | | | | | | | |
| | M | | | | | | | | | | | | |
| 961 | R.FQQGKDLQQYVSV | | 748.638 | 4 | 2991.532 | 2991.531 | 0 | 0.2 | 2828 | 360.0 | 360.0 | 360.0 | 4.79 |
| | VVLDEVGLAEDSPK. | | | | | | | | | | | | |
| | M | | | | | | | | | | | | |
| 1171 | K.DLQQYVSVVVLDE | | 718.882 | 4 | 2872.505 | 2872.501 | 0 | 1.2 | 2833 | 859.8 | 859.8 | 859.8 | 14.78 |
| | VGLAEDSPKMPLK.T | | | | | | | | | | | | |
| 1171 | K.DLQQYVSVVVLDE | | 718.882 | 4 | 2872.505 | 2872.501 | 0 | 1.3 | 2833 | 749.6 | 749.6 | 749.6 | 11.79 |
| | VGLAEDSPKMPLK.T | | | | | | | | | | | | |
| 1171 | K.DLQQYVSVVVLDE | | 958.173 | 3 | 2872.504 | 2872.501 | 0 | 1.1 | 2833 | 694.9 | 694.9 | 694.9 | 11.39 |
| | VGLAEDSPKMPLK.T | | | | | | | | | | | | |
| 1171 | K.DLQQYVSVVVLDE | | 1436.758 | 2 | 2872.508 | 2872.501 | 0 | 2.5 | 2833 | 656.6 | 656.6 | 656.6 | 10.15 |
| | VGLAEDSPKMPLK.T | | | | | | | | | | | | |
| 932 | K.DLQQYVSVVVLDE | | 1202.120 | 2 | 2403.232 | 2403.229 | 0 | 1.2 | 2833 | 599.7 | 599.7 | 599.7 | 9.96 |
| | VGLAEDSPK.M | | | | | | | | | | | | |
| 932 | K.DLQQYVSVVVLDE | | 801.748 | 3 | 2403.231 | 2403.229 | 0 | 0.7 | 2833 | 487.0 | 487.0 | 487.0 | 8.32 |
| | VGLAEDSPK.M | | | | | | | | | | | | |
| 932 | K.DLQQYVSVVVLDE | | 801.749 | 3 | 2403.232 | 2403.229 | 0 | 1.1 | 2833 | 421.5 | 421.5 | 421.5 | 6.99 |
| | VGLAEDSPK.M | | | | | | | | | | | | |
| 1686 | K.MPLKTLHPLLEDG | C[+57] | 689.355 | 4 | 2754.397 | 2754.395 | 0 | 0.4 | 2855 | 788.6 | 788.6 | 788.6 | 12.08 |
| | C[+57.021]IEDDP | | | | | | | | | | | | |
| | APHKK.V | | | | | | | | | | | | |
| 1686 | K.MPLKTLHPLLEDG | C[+57] | 689.354 | 1 | 2754.396 | 2754.395 | 0 | 0.1 | 2855 | 745.3 | 745.3 | 745.3 | 11.08 |
| | C[+57.021]IEDDP | | | | | | | | | | | | |
| | APHKK.V | | | | | | | | | | | | |
| 1723 | K.TLHPLLEDGC | C[+57] | 572.036 | 4 | 2285.122 | 2285.123 | 0 | 0.4 | 2859 | 867.3 | 867.3 | 867.3 | 15.05 |
| | [+57.021] | | | | | | | | | | | | |
| | IEDDPAPHK | | | | | | | | | | | | |
| | K.V | | | | | | | | | | | | |
| 1723 | K.TLHPLLEDGC | C[+57] | 572.036 | 4 | 2285.122 | 2285.123 | 0 | −0.3 | 2859 | 866.1 | 866.1 | 866.1 | 15.05 |
| | [+57.021] | | | | | | | | | | | | |
| | IEDDPAPHK | | | | | | | | | | | | |
| | K.V | | | | | | | | | | | | |
| 1723 | K.TLHPLLEDGC | C[+57] | 762.380 | 3 | 2285.124 | 2285.123 | 0 | 0.6 | 2859 | 774.7 | 774.7 | 774.7 | 12.39 |
| | [+57.021] | | | | | | | | | | | | |
| | IEDDPAPHK | | | | | | | | | | | | |
| | K.V | | | | | | | | | | | | |
| 1723 | K.TLHPLLEDGC | C[+57] | 457.831 | 5 | 2285.124 | 2285.123 | 0 | 0.4 | 2859 | 660.2 | 660.2 | 660.2 | 11.77 |
| | [+57.021] | | | | | | | | | | | | |
| | IEDDPAPHK | | | | | | | | | | | | |
| | K.V | | | | | | | | | | | | |
| 1723 | K.TLHPLLEDGC | C[+57] | 457.831 | 5 | 2285.124 | 2285.123 | 0 | 0.4 | 2859 | 516.9 | 516.9 | 516.9 | 8.72 |
| | [+57.021] | | | | | | | | | | | | |
| | IEDDPAPHK | | | | | | | | | | | | |
| | K.V | | | | | | | | | | | | |
| 1723 | K.TLHPLLEDGC | C[+57] | 457.831 | 5 | 2285.124 | 2285.123 | 0 | 0.4 | 2859 | 497.9 | 497.9 | 497.9 | 7.99 |
| | [+57.021] | | | | | | | | | | | | |
| | IEDDPAPHK | | | | | | | | | | | | |
| | K.V | | | | | | | | | | | | |
| 1966 | K.KVGFVGISNWALD | | 567.982 | 3 | 1701.933 | 1701.932 | 0 | 0.2 | 2878 | 430.9 | 430.9 | 430.9 | 7.83 |
| | PAK.M | | | | | | | | | | | | |
| 1966 | K.KVGFVGISNWALD | | 851.469 | 2 | 1701.931 | 1701.932 | 0 | −0.6 | 2878 | 397.4 | 397.4 | 397.4 | 6.77 |
| | PAK.M | | | | | | | | | | | | |
| 4630 | K.KVGFVGISNWALD | M[+16] | 530.533 | 4 | 2119.110 | 2119.112 | 0 | −0.6 | 2878 | 332.0 | 332.0 | 332.0 | 4.80 |
| | PAKM[+15.995]NR | | | | | | | | | | | | |
| | .G | | | | | | | | | | | | |
| 1006 | K.VGFVGISNWALDP | | 659.347 | 3 | 1976.028 | 1975.022 | 1 | 1.2 | 2879 | 652.3 | 652.3 | 652.3 | 11.77 |
| | AKMNR.G | | | | | | | | | | | | |
| 836 | K.VGFVGISNWALDP | | 787.422 | 2 | 1573.836 | 1573.837 | 0 | 0.8 | 2879 | 648.0 | 648.0 | 648.0 | 10.26 |
| | AK.M | | | | | | | | | | | | |
| 836 | K.VGFVGISNWALDP | | 787.422 | 2 | 1573.838 | 1573.837 | 0 | 0.2 | 2879 | 645.3 | 645.3 | 1645.3 | 10.96 |
| | AK.M | | | | | | | | | | | | |
| 1006 | K.VGFVGISNWALDP | | 659.347 | 3 | 1976.025 | 1975.022 | 1 | −0.2 | 2879 | 640.5 | 640.5 | 398.4 | 10.77 |
| | AKMNR.G | | | | | | | | | | | | |
| 1006 | K.VGFVGISNWALDP | | 659.013 | 3 | 1975.025 | 1975.022 | 0 | 1.6 | 2879 | 628.0 | 628.0 | 628.0 | 10.77 |
| | AKMNR.G | | | | | | | | | | | | |
| 1006 | K.VGFVGISNWALDP | | 659.013 | 3 | 1975.024 | 1975.022 | 0 | 1.3 | 2879 | 618.6 | 618.6 | 618.6 | 10.77 |
| | AKMNR.G | | | | | | | | | | | | |
| 1006 | K.VGFVGISNWALDP | | 659.012 | 3 | 1975.022 | 1975.022 | 0 | 0.1 | 2879 | 600.9 | 600.9 | 600.9 | 10.77 |
| | AKMNR.G | | | | | | | | | | | | |
| 1006 | K.VGFVGISNWALDP | | 659.012 | 3 | 1975.022 | 1975.022 | 0 | 0.0 | 2879 | 591.3 | 586.8 | 1586.8 | 9.77 |
| | AKMNR.G | | | | | | | | | | | | |
| 1530 | K.VGFVGISNWALDP | M[+16] | 664.344 | 3 | 1991.018 | 1991.017 | 0 | 0.5 | 2879 | 567.2 | 567.2 | 567.2 | 9.77 |
| | AKM[+15.995]NR. | | | | | | | | | | | | |
| | G | | | | | | | | | | | | |
| 1006 | K.VGFVGISNWALDP | | 659.013 | 3 | 1975.024 | 1975.022 | 0 | 1.3 | 2879 | 553.2 | 553.2 | 553.2 | 8.93 |
| | AKMNR.G | | | | | | | | | | | | |
| 1486 | K.VGFVGISN | N[+1] | 787.915 | 2 | 1574.822 | 1574.821 | 0 | 0.4 | 2879 | 533.2 | 533.2 | 533.2 | 9.12 |
| | [+0.984] | | | | | | | | | | | | |
| | WALDPAK.M | | | | | | | | | | | | |
| 836 | K.VGFVGISNWALDP | | 525.284 | 3 | 1573.838 | 1573.837 | 0 | 0.2 | 2879 | 510.8 | 510.8 | 510.8 | 7.59 |
| | AK.M | | | | | | | | | | | | |
| 1530 | K.VGFVGISNWALDP | M[+16] | 664.344 | 3 | 1991.018 | 1991.017 | 0 | 0.5 | 2879 | 504.3 | 504.3 | 504.3 | 8.72 |
| | AKM[+15.995]NR. | | | | | | | | | | | | |
| | G | | | | | | | | | | | | |
| 1006 | K.VGFVGISNWALDP | | 659.013 | 3 | 1975.025 | 1975.022 | 0 | 1.4 | 2879 | 493.1 | 489.8 | 489.8 | 7.99 |
| | AKMNR.G | | | | | | | | | | | | |
| 836 | K.VGFVGISNWALDP | | 787.422 | 2 | 1573.837 | 1573.837 | 0 | 0.2 | 2879 | 491.7 | 491.7 | 491.7 | 8.91 |
| | AK.M | | | | | | | | | | | | |
| 836 | K.VGFVGISNWALDP | | 787.421 | 2 | 1573.835 | 1573.837 | 0 | −1.3 | 2879 | 479.2 | 479.2 | 479.2 | 17.96 |
| | AK.M | | | | | | | | | | | | |
| 836 | K.VGFVGISNWALDP | | 787.422 | 2 | 1573.837 | 1573.837 | 0 | −0.5 | 2879 | 460.2 | 460.2 | 460.2 | 7.96 |
| | AK.M | | | | | | | | | | | | |
| 1530 | K.VGFVGISNWALDP | M[+16] | 996.011 | 2 | 1991.015 | 1991.017 | 0 | −0.7 | 2879 | 452.1 | 452.1 | 452.1 | 7.77 |
| | AKM[+15.995]NR. | | | | | | | | | | | | |
| | G | | | | | | | | | | | | |
| 1530 | K.VGFVGISNWALDP | M[+16] | 996.013 | 2 | 1991.018 | 1991.017 | 0 | 0.8 | 2879 | 419.4 | 419.4 | 419.4 | 8.37 |
| | AKM[+15.995]NR. | | | | | | | | | | | | |
| | G | | | | | | | | | | | | |
| 1105 | R.GIFVSRGSPNETE | | 678.688 | 3 | 2034.050 | 2034.050 | 0 | 0.1 | 2897 | 587.3 | 587.3 | 587.3 | 9.77 |
| | LIESAK.G | | | | | | | | | | | | |
| 1105 | R.GIFVSRGSPNETE | | 678.688 | 3 | 2034.050 | 2034.050 | 0 | 0.0 | 2897 | 559.1 | 533.7 | 533.7 | 18.93 |
| | LIESAK.G | | | | | | | | | | | | |
| 990 | R.GSPNETELIESAK | | 687.841 | 2 | 1374.674 | 1374.675 | 0 | 0.3 | 2903 | 675.8 | 675.8 | 675.8 | 10.88 |
| | .G | | | | | | | | | | | | |
| 990 | R.GSPNETELIESAK | | 687.841 | 2 | 1374.675 | 1374.675 | 0 | 0.1 | 2903 | 668.0 | 668.0 | 668.0 | 11.59 |
| | .G | | | | | | | | | | | | |
| 990 | R.GSPNETELIESAK | | 458.896 | 3 | 1374.675 | 1374.675 | 0 | 0.0 | 2903 | 544.0 | 544.0 | 544.0 | 7.43 |
| | .G | | | | | | | | | | | | |
| 990 | R.GSPNETELIESAK | | 458.896 | 3 | 1374.675 | 1374.675 | 0 | 0.0 | 2903 | 441.9 | 441.9 | 441.9 | 6.73 |
| | .G | | | | | | | | | | | | |
| 990 | R.GSPNETELIESAK | | 458.896 | 3 | 1374.675 | 1374.675 | 0 | 0.0 | 2903 | 338.5 | 338.5 | 338.5 | 4.73 |
| | .G | | | | | | | | | | | | |
| 3340 | K.GIC[+57.021]S | C[+57] | 681.838 | 2 | 1362.668 | 1362.668 | 0 | 0.3 | 2916 | 628.6 | 628.6 | 628.6 | 9.88 |
| | SDILVQDR.V | | | | | | | | | | | | |
| 1862 | R.VQGYFASFAK.A | | 559.287 | 2 | 1117.568 | 1117.568 | 0 | 0.1 | 2928 | 453.1 | 453.1 | 453.1 | 7.33 |
| 877 | K.RQDKEFFGLR.D | | 432.567 | 3 | 1295.686 | 1295.686 | 0 | 0.3 | 2945 | 509.5 | 509.5 | 509.5 | 7.38 |
| 2123 | R.QDKEFFGLRDYYS | | 674.683 | 3 | 2022.034 | 2022.033 | 0 | 0.2 | 2946 | 395.2 | 395.2 | 395.2 | 6.74 |
| | LIK.M | | | | | | | | | | | | |
| 2123 | R.QDKEFFGLRDYYS | | 506.264 | | 2022.033 | 2022.033 | 0 | 0.1 | 2946 | 338.2 | 338.2 | 338.2 | 5.58 |
| | LIK.M | | | | | | | | | | | | |
| 2041 | R.DYYSLIK.M | | 451.237 | 2 | 901.467 | 901.467 | 0 | −0.1 | 2955 | 375.7 | 375.7 | 375.7 | 6.72 |
| 1007 | K.ASNRKPSPQDIAQ | | 617.678 | 3 | 1851.020 | 1851.020 | 0 | 0.2 | 2969 | 559.0 | 559.0 | 559.0 | 8.93 |
| | AVLR.N | | | | | | | | | | | | |
| 1007 | K.ASNRKPSPQDIAQ | | 617.678 | 3 | 1851.019 | 1851.020 | 0 | 0.5 | 2969 | 449.6 | 449.6 | 449.6 | 6.80 |
| | AVLR.N | | | | | | | | | | | | |
| 839 | R.KPSPQDIAQAVLR | | 474.940 | 3 | 1422.806 | 1422.806 | 0 | 0.2 | 2973 | 482.5 | 482.5 | 482.5 | 7.81 |
| | .N | | | | | | | | | | | | |
| 839 | R.KPSPQDIAQAVLR | | 474.940 | 3 | 1422.806 | 1422.806 | 0 | 0.3 | 2973 | 413.1 | 413.1 | 413.1 | 6.75 |
| | .N | | | | | | | | | | | | |
| 839 | R.KPSPQDIAQAVLR | | 474.940 | 3 | 1422.806 | 1422.806 | 0 | 0.0 | 2973 | 373.3 | 373.3 | 373.3 | 6.87 |
| | .N | | | | | | | | | | | | |
| 839 | R.KPSPQDIAQAVLR | | 475.275 | 3 | 1423.810 | 1422.806 | 1 | 0.1 | 2973 | 371.2 | 371.2 | 371.2 | 5.38 |
| | .N | | | | | | | | | | | | |
| 815 | R.NFSGKDDIQALDI | | 1210.129 | 2 | 2419.251 | 2419.250 | 0 | 0.4 | 2986 | 629.9 | 572.0 | 572.0 | 10.77 |
| | FLANLPEAK.C | | | | | | | | | | | | |
| 815 | R.NFSGKDDIQALDI | | 1210.131 | 2 | 2419.254 | 2419.250 | 0 | 1.6 | 2986 | 587.1 | 587.1 | 587.1 | 9.77 |
| | FLANLPEAK.C | | | | | | | | | | | | |
| 1490 | R.N[+0.984]FSGK | N[+1] | 807.417 | 3 | 2420.236 | 2420.234 | 0 | 0.7 | 2986 | 533.9 | 533.9 | 322.5 | 8.20 |
| | DDIQALDIFLANLPE | | | | | | | | | | | | |
| | AK.C | | | | | | | | | | | | |
| 815 | R.NFSGKDDIQALDI | | 807.088 | 3 | 2419.250 | 2419.250 | 0 | 0.2 | 2986 | 489.0 | 489.0 | 489.0 | 8.72 |
| | FLANLPEAK.C | | | | | | | | | | | | |
| 1490 | R.N[+0.984]FSGK | N[+1] | 807.417 | 3 | 2420.236 | 2420.234 | 0 | 0.7 | 2986 | 468.4 | 468.4 | 232.7 | 7.51 |
| | DDIQALDIFLANLPE | | | | | | | | | | | | |
| | AK.C | | | | | | | | | | | | |
| 815 | R.NFSGKDDIQALDI | | 807.088 | 3 | 2419.251 | 2419.250 | 0 | 0.1 | 2986 | 461.7 | 461.7 | 461.7 | 7.75 |
| | FLANLPEAK.C | | | | | | | | | | | | |
| 815 | R.NFSGKDDIQALDI | | 1210.129 | 2 | 2419.250 | 2419.250 | 0 | −0.3 | 2986 | 444.7 | 444.7 | 444.7 | 8.48 |
| | FLANLPEAK.C | | | | | | | | | | | | |
| 815 | R.NFSGKDDIQALDI | | 807.088 | 3 | 2419.251 | 2419.250 | 0 | 0.1 | 2986 | 384.7 | 384.7 | 384.7 | 7.53 |
| | FLANLPEAK.C | | | | | | | | | | | | |
| 815 | R.NFSGKDDIQALDI | | 807.089 | 3 | 2419.251 | 2419.250 | 0 | 0.3 | 2986 | 362.5 | 266.1 | 266.1 | 5.53 |
| | FLANLPEAK.C | | | | | | | | | | | | |
| 815 | R.NFSGKDDIQALDI | | 807.088 | 3 | 2419.250 | 2419.250 | 0 | −0.2 | 2986 | 326.5 | 326.5 | 326.5 | 5.66 |
| | FLANLPEAK.C | | | | | | | | | | | | |
| 815 | R.NFSGKDDIQALDI | | 807.090 | 3 | 2419.256 | 2419.250 | 0 | 2.2 | 2986 | 326.2 | 326.2 | 326.2 | 5.89 |
| | FLANLPEAK.C | | | | | | | | | | | | |
| 815 | R.NFSGKDDIQALDI | | 605.568 | 4 | 2419.250 | 2419.250 | 0 | 0.4 | 2986 | 310.4 | 310.4 | 310.4 | 4.75 |
| | FLANLPEAK.C | | | | | | | | | | | | |
| 876 | K.DDIQALDIFLANL | | 629.336 | 3 | 1885.993 | 1885.991 | 0 | 1.1 | 2991 | 488.1 | 488.1 | 488.1 | 8.32 |
| | PEAK.C | | | | | | | | | | | | |
| 1717 | K.C[+57.021]SEE | C[+57] | 710.844 | | 1420.681 | 1420.681 | 0 | 0.1 | 3008 | 556.3 | 556.3 | 556.3 | 8.75 |
| | VSPMQLIK.Q | | | | | | | | | | | | |
| 1717 | K.C[+57.021]SEE | C[+57] | 710.844 | 2 | 1420.681 | 1420.681 | 0 | 0.0 | 3008 | 543.4 | 438.1 | 438.1 | 8.75 |
| | VSPMQLIK.Q | | | | | | | | | | | | |
| 1717 | K.C[+57.021]SEE | C[+57] | 474.232 | 3 | 1420.681 | 1420.681 | 0 | 0.4 | 3008 | 506.4 | 375.6 | 375.6 | 6.27 |
| | VSPMQLIK.Q | | | | | | | | | | | | |
| 1166 | K.QNIFGPSQKVPGG | | 758.365 | 3 | 2273.079 | 2273.079 | 0 | 0.2 | 3020 | 821.0 | 821.0 | 821.0 | 14.78 |
| | EQEDAESR.Y | | | | | | | | | | | | |
| 1166 | K.QNIFGPSQKVPGG | | 758.364 | 3 | 2273.078 | 2273.079 | 0 | −0.4 | 3020 | 763.6 | 763.6 | 763.6 | 13.06 |
| | EQEDAESR.Y | | | | | | | | | | | | |
| 940 | K.QNIFGPSQKVPGG | | 776.657 | 4 | 3103.605 | 3103.606 | 0 | 0.3 | 3020 | 691.6 | 691.6 | 691.6 | 10.37 |
| | EQEDAESRYLLVLTK | | | | | | | | | | | | |
| | .N | | | | | | | | | | | | |
| 940 | K.QNIFGPSQKVPGG | | 1035.207 | 3 | 3103.606 | 3103.606 | 0 | 0.0 | 3020 | 682.2 | 682.2 | 1682.2 | 11.08 |
| | EQEDAESRYLLVLTK | | | | | | | | | | | | |
| | .N | | | | | | | | | | | | |
| 940 | K.QNIFGPSQKVPGG | | 1035.208 | 3 | 3103.609 | 3103.606 | 0 | 1.0 | 3020 | 615.4 | 615.4 | 615.4 | 10.08 |
| | EQEDAESRYLLVLTK | | | | | | | | | | | | |
| | .N | | | | | | | | | | | | |
| 1166 | K.QNIFGPSQKVPGG | | 1137.044 | 2 | 2273.081 | 2273.079 | 0 | 0.5 | 3020 | 565.2 | 565.2 | 565.2 | 9.77 |
| | EQEDAESR.Y | | | | | | | | | | | | |
| 940 | K.QNIFGPSQKVPGG | | 776.657 | 4 | 3103.606 | 3103.606 | 0 | 0.1 | 3020 | 506.1 | 506.1 | 506.1 | 8.03 |
| | EQEDAESRYLLVLTK | | | | | | | | | | | | |
| | .N | | | | | | | | | | | | |
| 940 | K.QNIFGPSQKVPGG | | 776.657 | 4 | 3103.605 | 3103.606 | 0 | 0.3 | 3020 | 495.8 | 495.8 | 495.8 | 7.32 |
| | EQEDAESRYLLVLTK | | | | | | | | | | | | |
| | .N | | | | | | | | | | | | |
| 1950 | R.YLLVLTK.N | | 425.276 | 2 | 849.545 | 849.544 | 0 | 0.4 | 3041 | 387.6 | 387.6 | 387.6 | 6.54 |
| 1732 | K.NYVALQILQQTFF | C[+57] | 1127.806 | 4 | 4508.202 | 4508.187 | 0 | 3.4 | 3048 | 831.9 | 831.9 | 831.9 | 12.13 |
| | EGDQQPEIIFGSGFP | | | | | | | | | | | | |
| | KDQEYTQLC | | | | | | | | | | | | |
| | [+57.021]R.N | | | | | | | | | | | | |
| 957 | K.NYVALQILQQTFF | | 1105.564 | 3 | 3314.676 | 3314.673 | 0 | 0.9 | 3048 | 700.5 | 641.0 | 641.0 | 11.98 |
| | EGDQQPEIIFGSGFP | | | | | | | | | | | | |
| | K.D | | | | | | | | | | | | |
| 957 | K.NYVALQILQQTFF | | 829.425 | 4 | 3314.676 | 3314.673 | 0 | 0.8 | 3048 | 669.7 | 669.7 | 669.7 | 10.98 |
| | EGDQQPEIIFGSGFP | | | | | | | | | | | | |
| | K.D | | | | | | | | | | | | |
| 957 | K.NYVALQILQQTFF | | 1105.564 | 3 | 3314.677 | 3314.673 | 0 | 1.2 | 3048 | 459.9 | 459.9 | 459.9 | 16.33 |
| | EGDQQPEIIFGSGFP | | | | | | | | | | | | |
| | K.D | | | | | | | | | | | | |
| 4389 | K.HYLDINTVLEKWQ | | 596.321 | 3 | 1786.949 | 1786.949 | 0 | 0.0 | 3172 | 309.7 | 309.7 | 309.7 | 5.58 |
| | K.S | | | | | | | | | | | | |
| 4631 | R.QGPRALTEELHQK | | 502.939 | 3 | 1506.802 | 1506.802 | 0 | 0.2 | 3237 | 322.9 | 162.5 | 162.5 | 4.67 |
| | .V | | | | | | | | | | | | |
| 872 | R.ALTEELHQKVSEE | | 856.446 | 2 | 1711.885 | 1711.886 | 0 | 0.8 | 3241 | 826.4 | 826.4 | 826.4 | 12.68 |
| | AK.S | | | | | | | | | | | | |
| 872 | R.ALTEELHQKVSEE | | 856.446 | 2 | 1711.884 | 1711.886 | 0 | −1.0 | 3241 | 645.0 | 645.0 | 645.0 | 8.68 |
| | AK.S | | | | | | | | | | | | |
| 872 | R.ALTEELHQKVSEE | | 428.727 | 4 | 1711.886 | 1711.886 | 0 | −0.1 | 3241 | 540.2 | 540.2 | 540.2 | 8.93 |
| | AK.S | | | | | | | | | | | | |
| 872 | R.ALTEELHQKVSEE | | 428.976 | 4 | 1712.884 | 1711.886 | 1 | −3.3 | 3241 | 476.7 | 476.7 | 476.7 | 7.16 |
| | AK.S | | | | | | | | | | | | |
| 872 | R.ALTEELHQKVSEE | | 428.727 | 4 | 1711.886 | 1711.886 | 0 | −0.1 | 3241 | 437.4 | 320.0 | 320.0 | 5.97 |
| | AK.S | | | | | | | | | | | | |
| 1695 | K.SILLNC | C[+57] | 764.911 | 2 | 1528.816 | 1528.815 | 0 | 0.2 | 3256 | 666.9 | 618.0 | 618.0 | 11.96 |
| | [+57.021] | | | | | | | | | | | | |
| | ATPDAVVR.L | | | | | | | | | | | | |
| 1173 | R.LSAYSLGGFAAEW | | 811.395 | 3 | 2432.169 | 2432.167 | 0 | 0.8 | 3270 | 933.6 | 933.6 | 933.6 | 30.00 |
| | LSQEYFHR.Q | | | | | | | | | | | | |
| 1173 | R.LSAYSLGGFAAEW | | 811.395 | 3 | 2432.169 | 2432.167 | 0 | 0.9 | 3270 | 891.3 | 891.3 | 891.3 | 15.95 |
| | LSQEYFHR.Q | | | | | | | | | | | | |
| 1173 | R.LSAYSLGGFAAEW | | 608.798 | 4 | 2432.168 | 2432.167 | 0 | 0.5 | 3270 | 787.2 | 787.2 | 1787.2 | 13.37 |
| | LSQEYFHR.Q | | | | | | | | | | | | |
| 1173 | R.LSAYSLGGFAAEW | | 811.395 | 3 | 2432.172 | 2432.167 | 0 | 1.9 | 3270 | 751.6 | 681.3 | 681.3 | 13.96 |
| | LSQEYFHR.Q | | | | | | | | | | | | |
| 1173 | R.LSAYSLGGFAAEW | | 811.395 | 3 | 2432.169 | 2432.167 | 0 | 1.0 | 3270 | 627.2 | 627.2 | 627.2 | 10.96 |
| | LSQEYFHR.Q | | | | | | | | | | | | |
| 1173 | R.LSAYSLGGFAAEW | | 1216.588 | 2 | 2432.169 | 2432.167 | 0 | 1.0 | 3270 | 614.3 | 614.3 | 614.3 | 10.96 |
| | LSQEYFHR.Q | | | | | | | | | | | | |
| 1005 | R.QRHNSFADFLQAH | | 627.314 | 4 | 2506.234 | 2506.233 | 0 | 0.2 | 3291 | 676.1 | 676.1 | 676.1 | 10.39 |
| | LHTADLER.H | | | | | | | | | | | | |
| 1005 | R.QRHNSFADFLQAH | | 627.314 | 4 | 2506.234 | 2506.233 | 0 | 0.2 | 3291 | 616.4 | 616.4 | 616.4 | 8.66 |
| | LHTADLER.H | | | | | | | | | | | | |
| 1005 | R.QRHNSFADFLQAH | | 502.053 | 5 | 2506.234 | 2506.233 | 0 | 0.2 | 3291 | 553.3 | 553.3 | 553.3 | 8.93 |
| | LHTADLER.H | | | | | | | | | | | | |
| 1005 | R.QRHNSFADFLQAH | | 836.083 | 3 | 2506.235 | 2506.233 | 0 | 0.7 | 3291 | 538.2 | 538.2 | 538.2 | 6.82 |
| | LHTADLER.H | | | | | | | | | | | | |
| 1005 | R.QRHNSFADFLQAH | | 836.083 | 3 | 2506.233 | 2506.233 | 0 | 0.1 | 3291 | 513.8 | 513.8 | 513.8 | 6.61 |
| | LHTADLER.H | | | | | | | | | | | | |
| 1005 | R.QRHNSFADFLQAH | | 418.545 | 6 | 2506.234 | 2506.233 | 0 | 0.3 | 3291 | 426.6 | 426.6 | 426.6 | 8.37 |
| | LHTADLER.H | | | | | | | | | | | | |
| 854 | R.HNSFADFLQAHLH | | 556.274 | 4 | 2222.074 | 2222.074 | 0 | 0.2 | 3293 | 796.5 | 796.5 | 796.5 | 13.96 |
| | TADLER.H | | | | | | | | | | | | |
| 854 | R.HNSFADFLQAHLH | | 556.274 | 4 | 2222.074 | 2222.074 | 0 | −0.1 | 3293 | 724.6 | 724.6 | 724.6 | 12.96 |
| | TADLER.H | | | | | | | | | | | | |
| 854 | R.HNSFADFLQAHLH | | 445.221 | 5 | 2222.075 | 2222.074 | 0 | 0.3 | 3293 | 565.9 | 565.9 | 565.9 | 9.96 |
| | TADLER.H | | | | | | | | | | | | |
| 854 | R.HNSFADFLQAHLH | | 445.221 | 5 | 2222.075 | 2222.074 | 0 | 0.4 | 3293 | 473.2 | 473.2 | 473.2 | 8.67 |
| | TADLER.H | | | | | | | | | | | | |
| 854 | R.HNSFADFLQAHLH | | 1111.541 | 2 | 2222.074 | 2222.074 | 0 | 0.1 | 3293 | 416.3 | 416.3 | 416.3 | 6.45 |
| | TADLER.H | | | | | | | | | | | | |
| 943 | R.HAIFTEITTFSR. | | 711.873 | 2 | 1422.738 | 1422.738 | 0 | 0.4 | 3312 | 548.3 | 548.3 | 548.3 | 8.75 |
| | L | | | | | | | | | | | | |
| 943 | R.HAIFTEITTFSR. | | 711.873 | 2 | 1422.738 | 1422.738 | 0 | 0.3 | 3312 | 483.7 | 483.7 | 483.7 | 7.81 |
| | L | | | | | | | | | | | | |
| 943 | R.HAIFTEITTFSR. | | 474.917 | 3 | 1422.738 | 1422.738 | 0 | 0.0 | 3312 | 427.7 | 427.7 | 427.7 | 7.46 |
| | L | | | | | | | | | | | | |
| 943 | R.HAIFTEITTFSR. | | 474.917 | 3 | 1422.737 | 1422.738 | 0 | −0.4 | 3312 | 413.9 | 413.9 | 413.9 | 6.51 |
| | L | | | | | | | | | | | | |
| 943 | R.HAIFTEITTFSR. | | 474.917 | 3 | 1422.737 | 1422.738 | 0 | 0.1 | 3312 | 402.2 | 402.2 | 402.2 | 7.46 |
| | L | | | | | | | | | | | | |
| 943 | R.HAIFTEITTFSR. | | 474.917 | 3 | 1422.737 | 1422.738 | 0 | 0.1 | 3312 | 319.6 | 319.6 | 319.6 | 5.40 |
| | L | | | | | | | | | | | | |
| 1143 | R.APKPTLLWLQQFD | | 703.129 | 1 | 2809.493 | 2809.492 | 0 | 0.4 | 3341 | 668.3 | 668.3 | 668.3 | 11.77 |
| | TEYSFLKEVR.N | | | | | | | | | | | | |
| 1143 | R.APKPTLLWLQQFD | | 937.169 | 3 | 2809.491 | 2809.492 | 0 | 0.4 | 3341 | 642.2 | 642.2 | 642.2 | 10.06 |
| | TEYSFLKEVR.N | | | | | | | | | | | | |
| 1143 | R.APKPTLLWLQQFD | | 937.503 | 3 | 2810.494 | 2809.492 | 1 | −0.5 | 3341 | 577.5 | 577.5 | 577.5 | 9.06 |
| | TEYSFLKEVR.N | | | | | | | | | | | | |
| 894 | R.APKPTLLWLQQFD | | 809.099 | 3 | 2425.281 | 2425.280 | 0 | 0.5 | 3341 | 475.4 | 475.4 | 475.4 | 7.70 |
| | TEYSFLK.E | | | | | | | | | | | | |
| 894 | R.APKPTLLWLQQFD | | 809.099 | 3 | 2425.281 | 2425.280 | 0 | 0.5 | 3341 | 333.3 | 333.3 | 333.3 | 5.86 |
| | TEYSFLK.E | | | | | | | | | | | | |
| 858 | K.ILIFQTDFEDGIR | | 812.777 | 3 | 2436.317 | 2436.313 | 0 | 1.3 | 3374 | 463.9 | 463.9 | 463.9 | 7.51 |
| | SAQLIASAK.Y | | | | | | | | | | | | |
| 2170 | K.ILIFQTDFEDGIR | | 783.912 | 2 | 1566.817 | 1566.816 | 0 | 0.4 | 3374 | 396.9 | 396.9 | 396.9 | 17.10 |
| | .S | | | | | | | | | | | | |
| 848 | K.YSVINEINK.I | | 540.290 | 2 | 1079.573 | 1079.573 | 0 | 0.0 | 3396 | 498.2 | 498.2 | 498.2 | 7.34 |
| 855 | R.RSTLMVSDVTR.L | | 422.228 | 3 | 1264.668 | 1264.668 | 0 | 0.1 | 3447 | 505.4 | 505.4 | 505.4 | 7.57 |
| 855 | R.RSTLMVSDVTR.L | | 422.227 | 3 | 1264.668 | 1264.668 | 0 | 0.2 | 3447 | 493.1 | 493.1 | 493.1 | 7.57 |
| 1242 | R.STLMVSDVTRLQH | | 732.790 | 5 | 3659.920 | 3659.921 | 0 | 0.4 | 3448 | 705.0 | 705.0 | 705.0 | 11.68 |
| | VTISQLFAPGDLPEL | | | | | | | | | | | | |
| | GLEHR.A | | | | | | | | | | | | |
| 1242 | R.STLMVSDVTRLQH | | 732.790 | 5 | 3659.921 | 3659.921 | 0 | 0.1 | 3448 | 701.4 | 701.4 | 701.4 | 12.39 |
| | VTISQLFAPGDLPEL | | | | | | | | | | | | |
| | GLEHR.A | | | | | | | | | | | | |
| 1242 | R.STLMVSDVTRLQH | | 732.790 | 5 | 3659.920 | 3659.921 | 0 | −0.3 | 3448 | 677.4 | 677.4 | 677.4 | 10.68 |
| | VTISQLFAPGDLPEL | | | | | | | | | | | | |
| | GLEHR.A | | | | | | | | | | | | |
| 1242 | R.STLMVSDVTRLQH | | 915.736 | 4 | 3659.921 | 3659.921 | 0 | 0.0 | 3448 | 630.3 | 630.3 | 630.3 | 10.39 |
| | VTISQLFAPGDLPEL | | | | | | | | | | | | |
| | GLEHR.A | | | | | | | | | | | | |
| 845 | R.STLMVSDVTR.L | | 554.787 | 2 | 1108.567 | 1108.567 | 0 | −0.2 | 3448 | 547.5 | 547.5 | 1547.5 | 8.02 |
| 1579 | R.STLM[+15.995] | M[+16] | 562.784 | 2. | 1124.562 | 1124.562 | 0 | 0.0 | 3448 | 494.4 | 494.4 | 494.4 | 8.54 |
| | VSDVTR.L | | | | | | | | | | | | |
| 845 | R.STLMVSDVTR.L | | 554.787 | 2 | 1108.567 | 1108.567 | 0 | −0.2 | 3448 | 482.0 | 336.6 | 336.6 | 6.29 |
| 845 | R.STLMVSDVTR.L | | 554.787 | 2 | 1108.567 | 1108.567 | 0 | −0.1 | 3448 | 369.4 | 369.4 | 369.4 | 6.72 |
| 1579 | R.STLM[+15.995] | M[+16] | 562.785 | 2 | 1124.562 | 1124.562 | 0 | 0.1 | 3448 | 365.3 | 365.3 | 365.3 | 6.72 |
| | VSDVTR.L | | | | | | | | | | | | |
| 845 | R.STLMVSDVTR.L | | 554.787 | 2 | 1108.567 | 1108.567 | 0 | −0.2 | 3448 | 352.7 | 305.0 | 305.0 | 5.57 |
| 845 | R.STLMVSDVTR.L | | 554.787 | 2 | 1108.567 | 1108.567 | 0 | 0.2 | 3448 | 319.6 | 319.6 | 319.6 | 5.24 |
| 845 | R.STLMVSDVTR.L | | 554.787 | 2 | 1108.567 | 1108.567 | 0 | 0.2 | 3448 | 305.8 | 305.8 | 305.8 | 5.40 |
| 979 | R.LQHVTISQLFAPG | | 643.349 | 4 | 2570.373 | 2570.373 | 0 | 0.2 | 3458 | 838.0 | 838.0 | 838.0 | 14.95 |
| | DLPELGLEHR.A | | | | | | | | | | | | |
| 1391 | R.LQHVTISQLFAPG | | 1073.339 | 6 | 6434.999 | 6435.004 | 0 | 0.7 | 3458 | 813.6 | 813.6 | 813.6 | 12.03 |
| | DLPELGLEHRAEDGH | | | | | | | | | | | | |
| | EEAMETEASTSGEVA | | | | | | | | | | | | |
| | EVAEEAMETESSEKV | | | | | | | | | | | | |
| | GK.E | | | | | | | | | | | | |
| 979 | R.LQHVTISQLFAPG | | 857.462 | 3 | 2570.371 | 2570.373 | 0 | −0.5 | 3458 | 795.5 | 795.5 | 795.5 | 13.26 |
| | DLPELGLEHR.A | | | | | | | | | | | | |
| 979 | R.LQHVTISQLFAPG | | 643.349 | 4 | 2570.373 | 2570.373 | 0 | 0.2 | 3458 | 721.3 | 721.3 | 721.3 | 12.96 |
| | DLPELGLEHR.A | | | | | | | | | | | | |
| 1391 | R.LQHVTISQLFAPG | | 1073.507 | 6 | 6436.008 | 6435.004 | 1 | 0.1 | 3458 | 687.0 | 687.0 | 687.0 | 9.74 |
| | DLPELGLEHRAEDGH | | | | | | | | | | | | |
| | EEAMETEASTSGEVA | | | | | | | | | | | | |
| | EVAEEAMETESSEKV | | | | | | | | | | | | |
| | GK.E | | | | | | | | | | | | |
| 979 | R.LQHVTISQLFAPG | | 857.796 | 3 | 2571.373 | 2570.373 | 1 | −1.2 | 3458 | 614.5 | 614.5 | 614.5 | 10.26 |
| | DLPELGLEHR.A | | | | | | | | | | | | |
| 835 | R.LQHVTISQLFAPG | | 1230.971 | 5 | 6150.824 | 6150.819 | 0 | 0.9 | 3458 | 572.2 | 572.2 | 572.2 | 7.62 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 835 | R.LQHVTISQLFAPG | | 1231.171 | 5 | 6151.825 | 6150.819 | 1 | 0.4 | 3458 | 565.8 | 565.8 | 565.8 | 17.39 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 835 | R.LQHVTISQLFAPG | | 1231.171 | 5 | 6151.825 | 6150.819 | 1 | 0.4 | 3458 | 548.4 | 548.4 | 548.4 | 16.40 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 979 | R.LQHVTISQLFAPG | | 1285.690 | 2 | 2570.373 | 2570.373 | 0 | 0.3 | 3458 | 516.6 | 516.6 | 516.6 | 7.53 |
| | DLPELGLEHR.A | | | | | | | | | | | | |
| 835 | R.LQHVTISQLFAPG | | 879.551 | 7 | 6150.816 | 6150.819 | 0 | −0.5 | 3458 | 452.8 | 224.7 | 224.7 | 2.97 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 835 | R.LQHVTISQLFAPG | | 1026.310 | 6 | 6152.821 | 6150.819 | 2 | −0.7 | 3458 | 391.3 | 148.2 | 148.2 | 2.68 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 835 | R.LQHVTISQLFAPG | | 879.695 | 7 | 6151.820 | 6150.819 | 1 | −0.4 | 3458 | 387.7 | 387.7 | 387.7 | 4.24 |
| | DLPELGLEHRAEDGH | | | | | | | | | | | | |
| | EEAMETEASTSGEVA | | | | | | | | | | | | |
| | EVAEEAMETESSEK. | | | | | | | | | | | | |
| | V | | | | | | | | | | | | |
| 979 | R.LQHVTISQLFAPG | | 857.462 | 3 | 2570.371 | 2570.373 | 0 | −0.5 | 3458 | 375.4 | 375.4 | 375.4 | 6.77 |
| | DLPELGLEHR.A | | | | | | | | | | | | |
| 954 | R.AEDGHEEAMETEA | | 1129.704 | 5 | 5644.489 | 5644.492 | 0 | −0.5 | 3481 | 805.8 | 805.8 | 805.8 | 12.58 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.904 | 5 | 5645.493 | 5644.492 | 1 | 0.4 | 3481 | 779.6 | 779.6 | 779.6 | 11.58 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.703 | 5 | 5644.488 | 5644.492 | 0 | 0.8 | 3481 | 765.0 | 765.0 | 765.0 | 11.58 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1411.879 | 4 | 5644.492 | 5644.492 | 0 | 0.1 | 3481 | 762.5 | 762.5 | 1762.5 | 12.28 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.705 | 5 | 5644.493 | 5644.492 | 0 | 0.2 | 3481 | 726.8 | 726.8 | 726.8 | 11.28 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 939 | R.AEDGHEEAMETEA | | 1295.890 | 3 | 3885.656 | 3883.649 | 2 | 0.0 | 3481 | 726.4 | 726.4 | 726.4 | 11.97 |
| | STSGEVAEVAEEAME | | | | | | | | | | | | |
| | TESSEKVGK.E | | | | | | | | | | | | |
| 939 | R.AEDGHEEAMETEA | | 971.668 | 4 | 3883.651 | 3883.649 | 0 | 0.3 | 3481 | 702.3 | 702.3 | 702.3 | 11.97 |
| | STSGEVAEVAEEAME | | | | | | | | | | | | |
| | TESSEKVGK.E | | | | | | | | | | | | |
| 939 | R.AEDGHEEAMETEA | | 971.668 | 4 | 3883.649 | 3883.649 | 0 | 0.0 | 3481 | 660.9 | 660.9 | 660.9 | 10.97 |
| | STSGEVAEVAEEAME | | | | | | | | | | | | |
| | TESSEKVGK.E | | | | | | | | | | | | |
| 948 | R.AEDGHEEAMETEA | | 1200.493 | 3 | 3599.466 | 3599.464 | 0 | 0.3 | 3481 | 635.1 | 635.1 | 635.1 | 10.58 |
| | STSGEVAEVAEEAME | | | | | | | | | | | | |
| | TESSEK.V | | | | | | | | | | | | |
| 948 | R.AEDGHEEAMETEA | | 1200.493 | 3 | 3599.464 | 3599.464 | 0 | 0.1 | 3481 | 620.2 | 620.2 | 620.2 | 10.58 |
| | STSGEVAEVAEEAME | | | | | | | | | | | | |
| | TESSEK.V | | | | | | | | | | | | |
| 948 | R.AEDGHEEAMETEA | | 1200.493 | 3 | 3599.465 | 3599.464 | 0 | 0.2 | 3481 | 611.3 | 611.3 | 611.3 | 10.58 |
| | STSGEVAEVAEEAME | | | | | | | | | | | | |
| | TESSEK.V | | | | | | | | | | | | |
| 948 | R.AEDGHEEAMETEA | | 1200.494 | 3 | 3599.467 | 3599.464 | 0 | 0.7 | 3481 | 609.0 | 609.0 | 609.0 | 10.58 |
| | STSGEVAEVAEEAME | | | | | | | | | | | | |
| | TESSEK.V | | | | | | | | | | | | |
| 954 | R.AEDGHEEAMETEA | | 1129.705 | 5 | 5644.495 | 5644.492 | 0 | 0.5 | 3481 | 605.5 | 605.5 | 605.5 | 8.08 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1411.879 | 4 | 5644.494 | 5644.492 | 0 | 0.4 | 3481 | 588.2 | 588.2 | 588.2 | 8.28 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.705 | 5 | 5644.496 | 5644.492 | 0 | 0.8 | 3481 | 588.0 | 588.0 | 588.0 | 7.31 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.706 | 5 | 5644.500 | 5644.492 | 0 | 1.4 | 3481 | 582.5 | 582.5 | 582.5 | 17.55 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.905 | 5 | 5645.497 | 5644.492 | 1 | 0.2 | 3481 | 570.5 | 570.5 | 570.5 | 7.31 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1411.879 | 4 | 5644.495 | 5644.492 | 0 | 0.6 | 3481 | 564.6 | 564.6 | 564.6 | 18.28 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 3260 | R.AEDGHEEAM | M[+16] | 1133.304 | 5 | 5662.493 | 5660.487 | 2 | 0.1 | 3481 | 524.6 | 524.6 | 211.3 | 5.91 |
| | [+15.995] | | | | | | | | | | | | |
| | ETEASTSGEV | | | | | | | | | | | | |
| | AEVAEEAMETESSEK | | | | | | | | | | | | |
| | VGKETSELGGSDVSI | | | | | | | | | | | | |
| | LDTTR.L | | | | | | | | | | | | |
| 954 | R.AEDGHEEAMETEA | | 941.755 | 6 | 5645.493 | 5644.492 | 1 | 0.5 | 3481 | 519.6 | 519.6 | 519.6 | 4.73 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 954 | R.AEDGHEEAMETEA | | 1129.905 | 5 | 5645.497 | 5644.492 | 1 | 0.2 | 3481 | 508.9 | 508.9 | 508.9 | 6.03 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 948 | R.AEDGHEEAMETEA | | 1200.495 | 3 | 3599.470 | 3599.464 | 0 | 1.6 | 3481 | 465.7 | 465.7 | 465.7 | 7.08 |
| | STSGEVAEVAEEAME | | | | | | | | | | | | |
| | TESSEK.V | | | | | | | | | | | | |
| 954 | R.AEDGHEEAMETEA | | 1129.909 | 5 | 5645.515 | 5644.492 | 1 | 3.5 | 3481 | 418.4 | 418.4 | 418.4 | 4.11 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 948 | R.AEDGHEEAMETEA | | 900.622 | 4 | 3599.465 | 3599.464 | 0 | 0.3 | 3481 | 417.3 | 417.3 | 417.3 | 6.22 |
| | STSGEVAEVAEEAME | | | | | | | | | | | | |
| | TESSEK.V | | | | | | | | | | | | |
| 954 | R.AEDGHEEAMETEA | | 1129.708 | 5 | 5644.510 | 5644.492 | 0 | 3.1 | 3481 | 415.8 | 415.8 | 415.8 | 14.11 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 3072 | R.AEDGHEEAMETEA | S[+80] | 1145.699 | 5 | 5724.464 | 5724.458 | 0 | 1.0 | 3481 | 369.3 | 369.3 | 1.4 | 5.41 |
| | S[+79.966]TSGEV | | | | | | | | | | | | |
| | AEVAEEAMETESSEK | | | | | | | | | | | | |
| | VGKETSELGGSDVSI | | | | | | | | | | | | |
| | LDTTR.L | | | | | | | | | | | | |
| 939 | R.AEDGHEEAMETEA | | 972.170 | 4 | 3885.658 | 3883.649 | 2 | 0.4 | 3481 | 362.7 | 362.7 | 362.7 | 5.92 |
| | STSGEVAEVAEEAME | | | | | | | | | | | | |
| | TESSEKVGK.E | | | | | | | | | | | | |
| 4632 | R.AEDGHEEAMETEA | S[+80] | 954.916 | 6 | 5724.459 | 5724.458 | 0 | 0.0 | 3481 | 337.1 | 337.1 | 5.5 | 3.32 |
| | STS[+79.966]GEV | | | | | | | | | | | | |
| | AEVAEEAMETESSEK | | | | | | | | | | | | |
| | VGKETSELGGSDVSI | | | | | | | | | | | | |
| | LDTTR.L | | | | | | | | | | | | |
| 954 | R.AEDGHEEAMETEA | | 1129.704 | 5 | 5644.491 | 5644.492 | 0 | −0.2 | 3481 | 335.6 | 335.6 | 335.6 | 3.77 |
| | STSGEVAEVAEEAME | | | | | | | | | | | | |
| | TESSEKVGKETSELG | | | | | | | | | | | | |
| | GSDVSILDTTR.L | | | | | | | | | | | | |
| 988 | K.VGKETSELGGSDV | | 1032.527 | 2 | 2064.046 | 2064.046 | 0 | 0.2 | 3515 | 709.9 | 709.9 | 709.9 | 12.77 |
| | SILDTTR.L | | | | | | | | | | | | |
| 1119 | K.VGKETSELGGSDV | | 612.334 | 4 | 2446.314 | 2446.315 | 0 | 0.1 | 3515 | 552.2 | 552.2 | 552.2 | 18.62 |
| | SILDTTRLLR.S | | | | | | | | | | | | |
| 988 | K.VGKETSELGGSDV | | 688.687 | 3 | 2064.046 | 2064.046 | 0 | 0.3 | 3515 | 511.8 | 511.8 | 511.8 | 8.72 |
| | SILDTTR.L | | | | | | | | | | | | |
| 827 | K.ETSELGGSDVSIL | | 890.434 | 2 | 1779.860 | 1779.861 | 0 | −0.5 | 3518 | 648.9 | 648.9 | 648.9 | 10.26 |
| | DTTR.L | | | | | | | | | | | | |
| 827 | K.ETSELGGSDVSIL | | 593.959 | 3 | 1779.862 | 1779.861 | 0 | 0.6 | 3518 | 507.8 | 507.8 | 507.8 | 7.59 |
| | DTTR.L | | | | | | | | | | | | |
| 827 | K.ETSELGGSDVSIL | | 890.432 | 2 | 1779.857 | 1779.861 | 0 | −2.3 | 3518 | 449.9 | 449.9 | 449.9 | 17.96 |
| | DTTR.L | | | | | | | | | | | | |
| 1679 | R.SC[+57.021]VQ | C[+57] | 733.664 | 3 | 2198.977 | 2197.975 | 1 | −0.4 | 3538 | 533.5 | 533.5 | 469.8 | 8.23 |
| | SAVGMLRDQNESC | *2 | | | | | | | | | | | |
| | [+57.021]TR.N | | | | | | | | | | | | |
| 1713 | R.SC[+57.021]VQ | C[+57] | 604.300 | 2 | 1207.593 | 1207.592 | 0 | 0.3 | 3538 | 529.2 | 483.9 | 483.9 | 8.02 |
| | SAVGMLR.D | | | | | | | | | | | | |
| 1727 | R.VVLLLGLLNEDDA | C[+57] | 752.401 | 3 | 2255.187 | 2255.185 | 0 | 0.7 | 3561 | 455.0 | 455.0 | 455.0 | 7.94 |
| | C[+57.021]HASFL | | | | | | | | | | | | |
| | R.V | | | | | | | | | | | | |
| 1727 | R.VVLLLGLLNEDDA | C[+57] | 752.401 | 3 | 2255.187 | 2255.185 | 0 | 0.9 | 3561 | 403.4 | 403.4 | 403.4 | 17.59 |
| | C[+57.021]HASFL | | | | | | | | | | | | |
| | R.V | | | | | | | | | | | | |
| 828 | R.LSVFLKKQEESQF | | 517.884 | 5 | 2585.389 | 2585.388 | 0 | 0.4 | 3586 | 449.0 | 449.0 | 449.0 | 7.20 |
| | HPLEWLAR.E | | | | | | | | | | | | |
| 1038 | K.KQEESQFHPLEWL | | 633.324 | | 31897.958 | 1897.956 | 0 | 1.3 | 3592 | 565.8 | 565.8 | 565.8 | 9.96 |
| | AR.E | | | | | | | | | | | | |
| 859 | K.QEESQFHPLEWLA | | 590.625 | 3 | 1769.860 | 1769.861 | 0 | −0.3 | 3593 | 555.3 | 555.3 | 555.3 | 9.12 |
| | R.E | | | | | | | | | | | | |
| 859 | K.QEESQFHPLEWLA | | 885.434 | 2 | 1769.861 | 1769.861 | 0 | 0.3 | 3593 | 470.3 | 470.3 | 470.3 | 8.67 |
| | R.E | | | | | | | | | | | | |
| 1739 | R.EAC[+57.021]N | C[+57] | 855.381 | 2 | 1709.754 | 1709.755 | 0 | −0.6 | 3607 | 637.6 | 637.6 | 637.6 | 10.26 |
| | QDALQEAGTFR.H | | | | | | | | | | | | |
| 2234 | R.VQGAVTPLLASMI | | 640.021 | 3 | 1918.047 | 1918.047 | 0 | 0.3 | 3628 | 436.8 | 436.8 | 436.8 | 7.24 |
| | SFIDR.D | | | | | | | | | | | | |
| 1214 | K.IKDYLEELWVQAQ | | 908.479 | 3 | 2723.422 | 2722.397 | 1 | 7.7 | 3715 | 618.4 | 618.4 | 618.4 | 9.42 |
| | YITDAEGLPK.K | | | | | | | | | | | | |
| 1094 | K.IKDYLEELWVQAQ | | 713.380 | 4 | 2850.497 | 2850.492 | 0 | 1.7 | 3715 | 529.6 | 529.6 | 529.6 | 17.89 |
| | YITDAEGLPKK.F | | | | | | | | | | | | |
| 1856 | K.KFVDIFQQTPLGR | | 516.956 | 3 | 1548.853 | 1548.853 | 0 | 0.0 | 3738 | 409.9 | 409.9 | 409.9 | 7.22 |
| | .F | | | | | | | | | | | | |
| 1856 | K.KFVDIFQQTPLGR | | 517.291 | 3 | 1549.857 | 1548.853 | 1 | 0.1 | 3738 | 357.3 | 357.3 | 357.3 | 3.76 |
| | .F | | | | | | | | | | | | |
| 1379 | R.KLKAASEAPEEEV | | 688.617 | 4 | 2751.448 | 2751.446 | 0 | 0.4 | 3797 | 755.0 | 755.0 | 755.0 | 13.77 |
| | SLPWVHLAYQR.F | | | | | | | | | | | | |
| 1379 | R.KLKAASEAPEEEV | | 688.617 | 4 | 2751.448 | 2751.446 | 0 | 0.4 | 3797 | 708.1 | 708.1 | 708.1 | 12.77 |
| | SLPWVHLAYQR.F | | | | | | | | | | | | |
| 1379 | R.KLKAASEAPEEEV | | 688.868 | 4 | 2752.448 | 2751.446 | 1 | 0.7 | 3797 | 374.5 | 374.5 | 374.5 | 16.34 |
| | SLPWVHLAYQR.F | | | | | | | | | | | | |
| 1167 | K.LKAASEAPEEEVS | | 875.122 | 3 | 2623.351 | 2623.352 | 0 | 0.0 | 3798 | 849.1 | 849.1 | 849.1 | 14.78 |
| | LPWVHLAYQR.F | | | | | | | | | | | | |
| 1167 | K.LKAASEAPEEEVS | | 656.593 | 4 | 2623.352 | 2623.352 | 0 | 0.0 | 3798 | 698.8 | 698.8 | 698.8 | 11.77 |
| | LPWVHLAYQR.F | | | | | | | | | | | | |
| 1167 | K.LKAASEAPEEEVS | | 656.594 | 4 | 2623.352 | 2623.352 | 0 | 0.3 | 3798 | 605.3 | 605.3 | 605.3 | 10.77 |
| | LPWVHLAYQR.F | | | | | | | | | | | | |
| 1273 | K.AASEAPEEEVSLP | | 794.729 | 3 | 2382.172 | 2382.173 | 0 | 0.0 | 3800 | 835.6 | 835.6 | 835.6 | 14.95 |
| | WVHLAYQR.F | | | | | | | | | | | | |
| 1273 | K.AASEAPEEEVSLP | | 794.729 | 3 | 2382.172 | 2382.173 | 0 | −0.1 | 3800 | 755.7 | 755.7 | 755.7 | 13.96 |
| | WVHLAYQR.F | | | | | | | | | | | | |
| 1273 | K.AASEAPEEEVSLP | | 1191.589 | 2 | 2382.171 | 2382.173 | 0 | 0.6 | 3800 | 649.7 | 649.7 | 649.7 | 10.26 |
| | WVHLAYQR.F | | | | | | | | | | | | |
| 947 | R.ILTIYPQVLHSLM | | 628.685 | 3 | 1884.041 | 1884.041 | 0 | 0.1 | 3831 | 622.0 | 622.0 | 622.0 | 10.96 |
| | EAR.W | | | | | | | | | | | | |
| 947 | R.ILTIYPQVLHSLM | | 628.686 | 3 | 1884.042 | 1884.041 | 0 | 0.3 | 3831 | 617.4 | 617.4 | 617.4 | 10.96 |
| | EAR.W | | | | | | | | | | | | |
| 1588 | R.ILTIYPQVLHSLM | M[+16] | 634.017 | 3 | 1900.037 | 1900.036 | 0 | 0.5 | 3831 | 539.3 | 539.3 | 539.3 | 9.12 |
| | [+15.995]EAR.W | | | | | | | | | | | | |
| 947 | R.ILTIYPQVLHSLM | | 628.686 | 3 | 1884.042 | 1884.041 | 0 | 0.5 | 3831 | 498.2 | 498.2 | 498.2 | 8.91 |
| | EAR.W | | | | | | | | | | | | |
| 1947 | R.ILTIYPQVLHSLM | | 628.686 | 3 | 1884.042 | 1884.041 | 0 | 0.4 | 3831 | 445.9 | 445.9 | 445.9 | 7.94 |
| | EAR.W | | | | | | | | | | | | |
| 947 | R.ILTIYPQVLHSLM | | 628.685 | 3 | 1884.041 | 1884.041 | 0 | 0.1 | 3831 | 310.3 | 310.3 | 310.3 | 6.00 |
| | EAR.W | | | | | | | | | | | | |
| 992 | R.NTLKPSPQAWLQL | | 575.002 | 3 | 1722.990 | 1722.990 | 0 | 0.1 | 3873 | 665.2 | 665.2 | 665.2 | 11.96 |
| | VK.N | | | | | | | | | | | | |
| 992 | R.NTLKPSPQAWLQL | | 575.001 | 3 | 1722.989 | 1722.990 | 0 | 0.5 | 3873 | 640.8 | 640.8 | 640.8 | 10.26 |
| | VK.N | | | | | | | | | | | | |
| 992 | R.NTLKPSPQAWLQL | | 861.998 | 2 | 1722.989 | 1722.990 | 0 | 0.4 | 3873 | 518.9 | 518.9 | 518.9 | 8.21 |
| | VK.N | | | | | | | | | | | | |
| 992 | R.NTLKPSPQAWLQL | | 575.002 | 3 | 1722.991 | 1722.990 | 0 | 0.5 | 3873 | 336.7 | 336.7 | 336.7 | 6.09 |
| | VK.N | | | | | | | | | | | | |
| 4633 | K.NLSMPLELIC | C[+57] | 986.483 | 3 | 2957.433 | 2956.433 | 1 | −0.9 | 3888 | 711.5 | 703.0 | 272.1 | 11.87 |
| | [+57.021]SD | | | | | | | | | | | | |
| | EHMQGSG | | | | | | | | | | | | |
| | SLAQAVIR.E | | | | | | | | | | | | |
| 2814 | R.IFSTALFVEHVLL | | 673.703 | 3 | 2019.093 | 2019.091 | 0 | 1.1 | 3923 | 355.0 | 355.0 | 355.0 | 6.09 |
| | GTESR.V | | | | | | | | | | | | |
| 1047 | R.VPELQGLVTEHVF | | 646.363 | 3 | 1937.075 | 1937.074 | 0 | 0.6 | 3941 | 611.8 | 611.8 | 611.8 | 10.96 |
| | LLDK.C | | | | | | | | | | | | |
| 1047 | R.VPELQGLVTEHVF | | 646.364 | 3 | 1937.076 | 1937.074 | 0 | 0.9 | 3941 | 596.7 | 596.7 | 596.7 | 9.96 |
| | LLDK.C | | | | | | | | | | | | |
| 1047 | R.VPELQGLVTEHVF | | 646.363 | 3 | 1937.074 | 1937.074 | 0 | 0.0 | 3941 | 330.8 | 330.8 | 330.8 | 5.86 |
| | LLDK.C | | | | | | | | | | | | |
| 3298 | K.C[+57.021]LRE | C[+57] | 562.275 | 4 | 2246.079 | 2246.081 | 0 | 0.6 | 3958 | 418.3 | 418.3 | 1418.3 | 6.38 |
| | NSDVKTHGPFEAVMR | | | | | | | | | | | | |
| | .T | | | | | | | | | | | | |
| 1090 | R.ENSDVKTHGPFEA | | 606.293 | 3 | 1816.865 | 1816.865 | 0 | 0.1 | 3961 | 594.7 | 594.7 | 594.7 | 9.77 |
| | VMR.T | | | | | | | | | | | | |
| 1090 | R.ENSDVKTHGPFEA | | 908.936 | 2 | 1816.865 | 1816.865 | 0 | 0.0 | 3961 | 594.6 | 594.6 | 594.6 | 8.39 |
| | VMR.T | | | | | | | | | | | | |
| 1090 | R.ENSDVKTHGPFEA | | 606.293 | 3 | 1816.864 | 1816.865 | 0 | −0.4 | 3961 | 571.2 | 571.2 | 1571.2 | 9.06 |
| | VMR.T | | | | | | | | | | | | |
| 1090 | R.ENSDVKTHGPFEA | | 909.437 | 2 | 1817.866 | 1816.865 | 1 | −1.0 | 3961 | 515.3 | 515.3 | 160.9 | 6.63 |
| | VMR.T | | | | | | | | | | | | |
| 1090 | R.ENSDVKTHGPFEA | | 454.972 | 4 | 1816.864 | 1816.865 | 0 | 0.1 | 3961 | 422.5 | 422.5 | 1422.5 | 7.64 |
| | VMR.T | | | | | | | | | | | | |
| 1090 | R.ENSDVKTHGPFEA | | 454.972 | 4 | 1816.865 | 1816.865 | 0 | −0.1 | 3961 | 405.6 | 405.6 | 405.6 | 7.17 |
| | VMR.T | | | | | | | | | | | | |
| 1090 | R.ENSDVKTHGPFEA | | 454.972 | 4 | 1816.865 | 1816.865 | 0 | 0.3 | 3961 | 388.2 | 388.2 | 1388.2 | 7.28 |
| | VMR.T | | | | | | | | | | | | |
| 4634 | R.ENSDVKTHGPFEA | M[+16] | 611.625 | 3 | 1832.861 | 1832.860 | 0 | 0.6 | 3961 | 336.5 | 336.5 | 336.5 | 5.51 |
| | VM[+15.995]R.T | | | | | | | | | | | | |
| 4634 | R.ENSDVKTHGPFEA | M[+16] | 611.625 | 3 | 1832.861 | 1832.860 | 0 | 0.6 | 3961 | 318.6 | 318.6 | 318.6 | 5.42 |
| | VM[+15.995]R.T | | | | | | | | | | | | |
| 825 | K.THGPFEAVMR.T | | 572.782 | 2 | 1144.557 | 1144.557 | 0 | 0.0 | 3967 | 529.5 | 529.5 | 529.5 | 8.02 |
| 825 | K.THGPFEAVMR.T | | 572.782 | 2 | 1144.556 | 1144.557 | 0 | −0.4 | 3967 | 466.6 | 466.6 | 466.6 | 6.62 |
| 825 | K.THGPFEAVMR.T | | 572.782 | 2 | 1144.557 | 1144.557 | 0 | −0.2 | 3967 | 432.7 | 432.7 | 432.7 | 6.99 |
| 825 | K.THGPFEAVMR.T | | 572.782 | 2 | 1144.556 | 1144.557 | 0 | −0.3 | 3967 | 432.7 | 432.7 | 432.7 | 6.51 |
| 856 | K.DNAPPEKEVIESL | | 790.438 | 3 | 2369.301 | 2369.296 | 0 | 1.9 | 4080 | 623.9 | 623.9 | 623.9 | 10.77 |
| | LSLLFVQK.G | | | | | | | | | | | | |
| 856 | K.DNAPPEKEVIESL | | 1185.155 | 2 | 2369.303 | 2369.296 | 0 | 2.9 | 4080 | 473.3 | 473.3 | 473.3 | 7.24 |
| | LSLLFVQK.G | | | | | | | | | | | | |
| 856 | K.DNAPPEKEVIESL | | 593.080 | 4 | 2369.299 | 2369.296 | 0 | 1.1 | 4080 | 458.0 | 458.0 | 458.0 | 7.88 |
| | LSLLFVQK.G | | | | | | | | | | | | |
| 2072 | K.SLSPFNDVVDKTP | | 596.328 | 3 | 1786.970 | 1786.970 | 0 | 0.0 | 4116 | 402.5 | 402.5 | 1402.5 | 7.64 |
| | VIR.S | | | | | | | | | | | | |
| 2072 | K.SLSPFNDVVDKTP | | 893.988 | 2 | 1786.968 | 1786.970 | 0 | −1.0 | 4116 | 365.1 | 365.1 | 365.1 | 6.57 |
| | VIR.S | | | | | | | | | | | | |
| 1408 | K.TSAYSRNDELNHL | | 707.326 | 3 | 2119.965 | 2119.964 | 0 | 0.3 | 4186 | 768.3 | 768.3 | 768.3 | 13.77 |
| | EEEGR.F | | | | | | | | | | | | |
| 936 | K.TSAYSRNDELNHL | | 627.808 | 4 | 2508.211 | 2508.211 | 0 | −0.2 | 4186 | 506.5 | 506.5 | 506.5 | 8.41 |
| | EEEGRFLK.A | | | | | | | | | | | | |
| 936 | K.TSAYSRNDELNHL | | 836.741 | 3 | 2508.209 | 2508.211 | 0 | −0.9 | 4186 | 503.3 | 503.3 | 503.3 | 5.59 |
| | EEEGRFLK.A | | | | | | | | | | | | |
| 1408 | K.TSAYSRNDELNHL | | 530.747 | 4 | 2119.964 | 2119.964 | 0 | 0.1 | 4186 | 440.3 | 440.3 | 440.3 | 7.51 |
| | EEEGR.F | | | | | | | | | | | | |
| 936 | K.TSAYSRNDELNHL | | 502.649 | 5 | 2509.215 | 2508.211 | 1 | 0.2 | 4186 | 431.3 | 431.3 | 89.2 | 7.09 |
| | EEEGRFLK.A | | | | | | | | | | | | |
| 1408 | K.TSAYSRNDELNHL | | 1060.485 | 2 | 2119.963 | 2119.964 | 0 | −0.3 | 4186 | 428.1 | 428.1 | 428.1 | 6.26 |
| | EEEGR.F | | | | | | | | | | | | |
| 936 | K.TSAYSRNDELNHL | | 502.448 | 5 | 2508.210 | 2508.211 | 0 | −0.6 | 4186 | 398.1 | 398.1 | 398.1 | 6.26 |
| | EEEGRFLK.A | | | | | | | | | | | | |
| 936 | K.TSAYSRNDELNHL | | 627.808 | 4 | 2508.212 | 2508.211 | 0 | 0.2 | 4186 | 395.9 | 395.9 | 395.9 | 6.97 |
| | EEEGRFLK.A | | | | | | | | | | | | |
| 1482 | K.TSAYSRNDELN | N[+1] | 502.645 | 5 | 2509.195 | 2509.195 | 0 | −0.3 | 4186 | 366.3 | 366.3 | 41.3 | 6.97 |
| | [+0.984]HLEE | | | | | | | | | | | | |
| | EGRFL | | | | | | | | | | | | |
| | K.A | | | | | | | | | | | | |
| 936 | K.TSAYSRNDELNHL | | 502.645 | 5 | 2509.197 | 2508.211 | 1 | −7.2 | 4186 | 345.9 | 345.9 | 24.8 | 4.10 |
| | EEEGRFLK.A | | | | | | | | | | | | |
| 936 | K.TSAYSRNDELNHL | | 627.809 | 4 | 2508.212 | 2508.211 | 0 | 0.4 | 4186 | 343.6 | 343.6 | 343.6 | 5.35 |
| | EEEGRFLK.A | | | | | | | | | | | | |
| 936 | K.TSAYSRNDELNHL | | 837.075 | 3 | 2509.211 | 2508.211 | 1 | −1.5 | 4186 | 337.8 | 283.0 | 261.0 | 3.12 |
| | EEEGRFLK.A | | | | | | | | | | | | |
| 1408 | K.TSAYSRNDELNHL | | 530.993 | 4 | 2120.949 | 2119.964 | 1 | −8.8 | 4186 | 321.7 | 321.7 | 6.0 | 4.41 |
| | EEEGR.F | | | | | | | | | | | | |
| 978 | R.NDELNHLEEEGRF | | 614.971 | 3 | 1842.898 | 1842.898 | 0 | 0.1 | 4192 | 731.5 | 731.5 | 731.5 | 12.77 |
| | LK.A | | | | | | | | | | | | |
| 1494 | R.N[+0.984]DELN | N[+1] | 615.299 | 3 | 1843.884 | 1843.882 | 0 | 0.9 | 4192 | 668.8 | 668.8 | 231.3 | 11.77 |
| | HLEEEGRFLK.A | | | | | | | | | | | | |
| 1494 | R.N[+0.984]DELN | N[+1] | 461.726 | 4 | 1843.882 | 1843.882 | 0 | −0.1 | 4192 | 550.5 | 550.5 | 135.4 | 8.93 |
| | HLEEEGRFLK.A | | | | | | | | | | | | |
| 978 | R.NDELNHLEEEGRF | | 461.480 | 4 | 1842.897 | 1842.898 | 0 | 0.4 | 4192 | 527.7 | 527.7 | 527.7 | 18.23 |
| | LK.A | | | | | | | | | | | | |
| 1496 | R.NDELN[+0.984] | N[+1] | 485.883 | 3 | 1455.634 | 1455.635 | 0 | 0.2 | 4192 | 514.8 | 389.0 | 313.1 | 8.54 |
| | HLEEEGR.F | | | | | | | | | | | | |
| 1488 | R.NDELN[+0.984] | N[+1] | 461.726 | 4 | 1843.882 | 1843.882 | 0 | −0.3 | 4192 | 507.0 | 507.0 | 174.3 | 18.72 |
| | HLEEEGRFLK.A | | | | | | | | | | | | |
| 1496 | R.NDELN[+0.984] | N[+1] | 485.883 | 3 | 1455.634 | 1455.635 | 0 | −0.4 | 4192 | 500.7 | 415.4 | 339.0 | 6.86 |
| | HLEEEGR.F | | | | | | | | | | | | |
| 807 | R.NDELNHLEEEGR. | | 485.555 | 3 | 1454.650 | 1454.651 | 0 | 0.5 | 4192 | 491.4 | 491.4 | 1491.4 | 7.10 |
| | F | | | | | | | | | | | | |
| 807 | R.NDELNHLEEEGR. | | 727.829 | 2 | 1454.651 | 1454.651 | 0 | 0.0 | 4192 | 478.9 | 478.9 | 478.9 | 7.57 |
| | F | | | | | | | | | | | | |
| 978 | R.NDELNHLEEEGRF | | 921.952 | 2. | 1842.898 | 1842.898 | 0 | 0.3 | 4192 | 477.5 | 477.5 | 477.5 | 16.39 |
| | LK.A | | | | | | | | | | | | |
| 807 | R.NDELNHLEEEGR. | | 485.555 | 3 | 1454.650 | 1454.651 | 0 | −0.4 | 4192 | 464.4 | 464.4 | 464.4 | 6.62 |
| | F | | | | | | | | | | | | |
| 978 | R.NDELNHLEEEGRF | | 461.480 | 4 | 1842.898 | 1842.898 | 0 | 0.1 | 4192 | 453.5 | 453.5 | 453.5 | 8.48 |
| | LK.A | | | | | | | | | | | | |
| 978 | R.NDELNHLEEEGRF | | 614.971 | 3 | 1842.898 | 1842.898 | 0 | −0.2 | 4192 | 412.4 | 412.4 | 412.4 | 7.40 |
| | LK.A | | | | | | | | | | | | |
| 978 | R.NDELNHLEEEGRF | | 614.971 | 3 | 1842.898 | 1842.898 | 0 | 0.1 | 4192 | 408.8 | 408.8 | 1408.8 | 7.64 |
| | LK.A | | | | | | | | | | | | |
| 1488 | R.NDELN[+0.984] | N[+1] | 615.299 | 3 | 1843.882 | 1843.882 | 0 | 0.0 | 4192 | 379.7 | 379.7 | 244.5 | 7.28 |
| | HLEEEGRFLK.A | | | | | | | | | | | | |
| 978 | R.NDELNHLEEEGRF | | 461.480 | 4 | 1842.898 | 1842.898 | 0 | 0.0 | 4192 | 361.0 | 361.0 | 361.0 | 7.28 |
| | LK.A | | | | | | | | | | | | |
| 807 | R.NDELNHLEEEGR. | | 485.555 | 3 | 1454.650 | 1454.651 | 0 | −0.2 | 4192 | 359.8 | 359.8 | 359.8 | 5.48 |
| | F | | | | | | | | | | | | |
| 1496 | R.NDELN[+0.984] | N[+1] | 485.883 | 3 | 1455.635 | 1455.635 | 0 | 0.5 | 4192 | 332.4 | 332.4 | 187.2 | 5.48 |
| | HLEEEGR.F | | | | | | | | | | | | |
| 831 | R.GREPANEASVEYL | | 673.336 | 3 | 2017.994 | 2017.994 | 0 | 0.3 | 4214 | 528.3 | 528.3 | 528.3 | 8.93 |
| | QEVAR.I | | | | | | | | | | | | |
| 831 | R.GREPANEASVEYL | | 1009.501 | 2 | 2017.996 | 2017.994 | 0 | 0.9 | 4214 | 460.5 | 460.5 | 460.5 | 8.48 |
| | QEVAR.I | | | | | | | | | | | | |
| 831 | R.GREPANEASVEYL | | 1009.500 | 2. | 2017.993 | 2017.994 | 0 | −0.2 | 4214 | 427.7 | 427.7 | 427.7 | 7.64 |
| | QEVAR.I | | | | | | | | | | | | |
| 831 | R.GREPANEASVEYL | | 673.336 | 3 | 2017.994 | 2017.994 | 0 | −0.1 | 4214 | 422.8 | 422.8 | 422.8 | 7.64 |
| | QEVAR.I | | | | | | | | | | | | |
| 831 | R.GREPANEASVEYL | | 505.254 | 4 | 2017.994 | 2017.994 | 0 | 0.2 | 4214 | 412.7 | 412.7 | 412.7 | 6.80 |
| | QEVAR.I | | | | | | | | | | | | |
| 4636 | R.GREPAN | N[+1] | 673.665 | 3 | 2018.979 | 2018.978 | 0 | 0.7 | 4214 | 343.4 | 343.4 | 118.6 | 5.51 |
| | [+0.984]E | | | | | | | | | | | | |
| | ASVEYLQEVAR.I | | | | | | | | | | | | |
| 927 | R.EPANEASVEYLQE | | 902.939 | 2 | 1804.870 | 1804.871 | 0 | −0.7 | 4216 | 490.1 | 490.1 | 490.1 | 8.21 |
| | VAR.I | | | | | | | | | | | | |
| 1624 | R.AADFLSEPEGGPE | M[+16] | 641.300 | 32 | 1921.885 | 1921.885 | 0 | 0.1 | 4239 | 682.0 | 682.0 | 682.0 | 11.77 |
| | M[+15.995]AKEK. | | | | | | | | | | | | |
| | Q | | | | | | | | | | | | |
| 929 | R.AADFLSEPEGGPE | | 824.880 | | 1648.752 | 1648.752 | 0 | 0.0 | 4239 | 679.7 | 679.7 | 679.7 | 11.96 |
| | MAK.E | | | | | | | | | | | | |
| 1232 | R.AADFLSEPEGGPE | | 635.968 | 3 | 1905.890 | 1905.890 | 0 | −0.1 | 4239 | 667.8 | 667.8 | 667.8 | 11.77 |
| | MAKEK.Q | | | | | | | | | | | | |
| 929 | R.AADFLSEPEGGPE | | 824.880 | 2 | 1648.753 | 1648.752 | 0 | 0.3 | 4239 | 655.0 | 655.0 | 655.0 | 11.96 |
| | MAK.E | | | | | | | | | | | | |
| 1232 | R.AADFLSEPEGGPE | | 953.449 | 2 | 1905.890 | 1905.890 | 0 | 0.1 | 4239 | 627.3 | 627.3 | 627.3 | 10.77 |
| | MAKEK.Q | | | | | | | | | | | | |
| 929 | R.AADFLSEPEGGPE | | 550.256 | 3 | 1648.753 | 1648.752 | 0 | 0.2 | 4239 | 482.5 | 482.5 | 482.5 | 7.34 |
| | MAK.E | | | | | | | | | | | | |
| 1624 | R.AADFLSEPEGGPE | M[+16] | 961.446 | 2 | 1921.885 | 1921.885 | 0 | 0.3 | 4239 | 402.7 | 395.0 | 395.0 | 8.37 |
| | M[+15.995]AKEK. | | | | | | | | | | | | |
| | Q | | | | | | | | | | | | |
| 1036 | R.GMEFVQGLSKPGR | | 809.091 | 3 | 2425.258 | 2425.260 | 0 | −0.8 | 4288 | 542.7 | 542.7 | 542.7 | 7.04 |
| | PHQWVFPK.D | | | | | | | | | | | | |
| 1036 | R.GMEFVQGLSKPGR | | 607.070 | 4 | 2425.260 | 2425.260 | 0 | 0.1 | 4288 | 438.1 | 438.1 | 438.1 | 8.56 |
| | PHQWVFPK.D | | | | | | | | | | | | |
| 1036 | R.GMEFVQGLSKPGR | | 485.858 | 5 | 2425.260 | 2425.260 | 0 | 0.0 | 4288 | 429.5 | 429.5 | 429.5 | 7.83 |
| | PHQWVFPK.D | | | | | | | | | | | | |
| 1036 | R.GMEFVQGLSKPGR | | 607.070 | 4 | 2425.260 | 2425.260 | 0 | −0.1 | 4288 | 379.1 | 379.1 | 379.1 | 7.47 |
| | PHQWVFPK.D | | | | | | | | | | | | |
| 1551 | R.GM[+15.995]EF | M[+16] | 611.069 | 4 | 2441.255 | 2441.255 | 0 | 0.3 | 4288 | 372.0 | 372.0 | 372.0 | 7.47 |
| | VQGLSKPGRPHQWVF | | | | | | | | | | | | |
| | PK.D | | | | | | | | | | | | |
| 1036 | R.GMEFVQGLSKPGR | | 607.070 | 4 | 2425.260 | 2425.260 | 0 | −0.1 | 4288 | 350.6 | 350.6 | 350.6 | 6.09 |
| | PHQWVFPK.D | | | | | | | | | | | | |
| 1551 | R.GM[+15.995]EF | M[+16] | 489.057 | 5 | 2441.255 | 2441.255 | 0 | 0.1 | 4288 | 320.5 | 320.5 | 320.5 | 6.09 |
| | VQGLSKPGRPHQWVF | | | | | | | | | | | | |
| | PK.D | | | | | | | | | | | | |
| 1551 | R.GM[+15.995]EF | M[+16] | 489.057 | 5 | 2441.255 | 2441.255 | 0 | −0.1 | 4288 | 312.8 | 312.8 | 312.8 | 5.77 |
| | VQGLSKPGRPHQWVF | | | | | | | | | | | | |
| | PK.D | | | | | | | | | | | | |
| 4637 | K.PGRPHQWVFPK.D | | 450.247 | 3 | 1348.728 | 1348.727 | 0 | 0.2 | 4298 | 387.6 | 387.6 | 387.6 | 6.54 |
| 1058 | K.DVVKQQGLRQDHP | | 703.022 | 3 | 2107.050 | 2107.046 | 0 | 1.8 | 4309 | 541.0 | 541.0 | 541.0 | 7.24 |
| | GQMDR.Y | | | | | | | | | | | | |
| 1527 | K.DVVKQQGLRQDHP | M[+16] | 708.352 | 3 | 2123.040 | 2123.041 | 0 | −0.4 | 4309 | 423.3 | 318.0 | 318.0 | 3.57 |
| | GQM[+15.995]DR. | | | | | | | | | | | | |
| | Y | | | | | | | | | | | | |
| 1058 | K.DVVKQQGLRQDHP | | 527.517 | 4 | 2107.046 | 2107.046 | 0 | −0.2 | 4309 | 325.7 | 325.7 | 325.7 | 5.35 |
| | GQMDR.Y | | | | | | | | | | | | |
| 1058 | K.DVVKQQGLRQDHP | | 422.216 | 5 | 2107.049 | 2107.046 | 0 | 1.2 | 4309 | 316.2 | 316.2 | 316.2 | 5.50 |
| | GQMDR.Y | | | | | | | | | | | | |
| 1527 | K.DVVKQQGLRQDHP | M[+16] | 531.516 | 4 | 2123.042 | 2123.041 | 0 | 0.3 | 4309 | 311.5 | 311.5 | 311.5 | 5.27 |
| | GQM[+15.995]DR. | | | | | | | | | | | | |
| | Y | | | | | | | | | | | | |
| 1880 | K.QQGLRQDHPGQMD | | 933.444 | 3 | 2798.318 | 2796.316 | 2 | −1.5 | 4313 | 398.2 | 398.2 | 398.2 | 5.13 |
| | RYLVYGDEYK.A | | | | | | | | | | | | |
| 1544 | K.QQGLRQDHPGQM | M[+16] | 704.084 | | :2813.316 | 2812.311 | 1 | 0.6 | 4313 | 370.2 | 370.2 | 370.2 | 6.74 |
| | [+15.995]DRYL | | | | | | | | | | | | |
| | VYGDEYK.A | | | | | | | | | | | | |
| 1544 | K.QQGLRQDHPGQM | M[+16] | 703.833 | 4 | 2812.308 | 2812.311 | 0 | 0.9 | 4313 | 333.6 | 333.6 | 333.6 | 4.65 |
| | [+15.995]DRYL | | | | | | | | | | | | |
| | VYGDEYK.A | | | | | | | | | | | | |
| 1880 | K.QQGLRQDHPGQMD | | 699.835 | 4 | 2796.317 | 2796.316 | 0 | 0.3 | 4313 | 327.4 | 327.4 | 1327.4 | 5.35 |
| | RYLVYGDEYK.A | | | | | | | | | | | | |
| 1880 | K.QQGLRQDHPGQMD | | 699.835 | 4 | 2796.317 | 2796.316 | 0 | 0.5 | 4313 | 325.8 | 325.8 | 325.8 | 5.35 |
| | RYLVYGDEYK.A | | | | | | | | | | | | |
| 1544 | K.QQGLRQDHPGQM | M[+16] | 563.268 | 5 | 2812.309 | 2812.311 | 0 | 0.5 | 4313 | 309.5 | 309.5 | 309.5 | 4.56 |
| | [+15.995]DRYLV | | | | | | | | | | | | |
| | YGDEYK.A | | | | | | | | | | | | |
| 3802 | R.QDHPGQMDRYLVY | | 511.648 | 5 | 2554.211 | 2554.214 | 0 | −1.2 | 4318 | 365.7 | 365.7 | 365.7 | 16.26 |
| | GDEYKALR.D | | | | | | | | | | | | |
| 3802 | R.QDHPGQMDRYLVY | | 511.649 | 5 | 2554.214 | 2554.214 | 0 | −0.3 | 4318 | 340.5 | 340.5 | 340.5 | 5.58 |
| | GDEYKALR.D | | | | | | | | | | | | |
| 816 | R.QDHPGQMDRYLVY | | 738.669 | 3 | 2213.994 | 2213.992 | 0 | 0.7 | 4318 | 308.1 | 308.1 | 308.1 | 5.42 |
| | GDEYK.A | | | | | | | | | | | | |
| 970 | R.YLVYGDEYKALRD | | 987.521 | 2 | 1974.034 | 1974.033 | 0 | 0.3 | 4327 | 515.0 | 515.0 | 515.0 | 7.03 |
| | AVAK.A | | | | | | | | | | | | |
| 1785 | R.YLVYGDEYK.A | | 575.277 | 2 | 1149.546 | 1149.546 | 0 | −0.1 | 4327 | 472.1 | 472.1 | 472.1 | 7.10 |
| 844 | R.YLVYGDEYKALR. | | 745.388 | 2 | 1489.768 | 1489.769 | 0 | −0.3 | 4327 | 465.3 | 465.3 | 465.3 | 6.67 |
| | D | | | | | | | | | | | | |
| 1785 | R.YLVYGDEYK.A | | 575.277 | 2 | 1149.546 | 1149.546 | 0 | 0.5 | 4327 | 437.6 | 437.6 | 437.6 | 6.28 |
| 970 | R.YLVYGDEYKALRD | | 494.264 | 4 | 1974.034 | 1974.033 | 0 | 0.7 | 4327 | 408.1 | 408.1 | 408.1 | 8.06 |
| | AVAK.A | | | | | | | | | | | | |
| 970 | R.YLVYGDEYKALRD | | 658.683 | 3 | 1974.034 | 1974.033 | 0 | 0.3 | 4327 | 391.3 | 391.3 | 391.3 | 6.97 |
| | AVAK.A | | | | | | | | | | | | |
| 1785 | R.YLVYGDEYK.A | | 575.277 | 2 | 1149.546 | 1149.546 | 0 | −0.2 | 4327 | 385.8 | 385.8 | 385.8 | 6.72 |
| 970 | R.YLVYGDEYKALRD | | 658.683 | 3 | 1974.034 | 1974.033 | 0 | 0.4 | 4327 | 369.6 | 369.6 | 369.6 | 6.74 |
| | AVAK.A | | | | | | | | | | | | |
| 970 | R.YLVYGDEYKALRD | | 494.264 | 4 | 1974.033 | 1974.033 | 0 | 0.0 | 4327 | 367.5 | 367.5 | 367.5 | 6.97 |
| | AVAK.A | | | | | | | | | | | | |
| 1785 | R.YLVYGDEYK.A | | 575.277 | 2 | 1149.546 | 1149.546 | 0 | −0.5 | 4327 | 330.7 | 330.7 | 330.7 | 14.62 |
| 1726 | K.AVLEC | C[+57] | 614.360 | 2 | 1227.713 | 1227.713 | 0 | 0.0 | 4344 | 589.5 | 589.5 | 589.5 | 8.86 |
| | [+57.021] | | | | | | | | | | | | |
| | KPLGIK.T | | | | | | | | | | | | |
| 1726 | K.AVLEC | C[+57] | 409.909 | 3 | 1227.713 | 1227.713 | 0 | 0.2 | 4344 | 584.6 | 584.6 | 584.6 | 9.59 |
| | [+57.021] | | | | | | | | | | | | |
| | KPLGIK.T | | | | | | | | | | | | |
| 1194 | K.TPQSQQSAYFLLT | | 950.503 | 2 | 1899.999 | 1899.996 | 0 | 1.4 | 4362 | 598.1 | 598.1 | 598.1 | 9.96 |
| | LFR.E | | | | | | | | | | | | |
| 1194 | K.TPQSQQSAYFLLT | | 634.004 | 3 | 1899.998 | 1899.996 | 0 | 0.8 | 4362 | 383.2 | 383.2 | 383.2 | 6.88 |
| | LFR.E | | | | | | | | | | | | |
| 1194 | K.TPQSQQSAYFLLT | | 634.005 | 3 | 1899.999 | 1899.996 | 0 | 1.5 | 4362 | 357.0 | 324.2 | 324.2 | 5.49 |
| | LFR.E | | | | | | | | | | | | |
| 1878 | R.EVAILYR.S | | 432.253 | 2 | 863.498 | 863.499 | 0 | −0.2 | 4378 | 426.7 | 426.7 | 426.7 | 6.99 |
| 1685 | R.SHNASLHPTPEQC | C[+57] | 664.646 | 3 | 1991.924 | 1991.924 | 0 | 0.1 | 4385 | 809.9 | 1809.9 | 809.9 | 14.95 |
| | [+57.021]EAVSK. | | | | | | | | | | | | |
| | F | | | | | | | | | | | | |
| 1685 | R.SHNASLHPTPEQC | C[+57] | 498.737 | 4 | 1991.924 | 1991.924 | 0 | 0.1 | 4385 | 457.8 | 457.8 | 457.8 | 17.94 |
| | [+57.021]EAVSK. | | | | | | | | | | | | |
| | F | | | | | | | | | | | | |
| 1685 | R.SHNASLHPTPEQC | C[+57] | 498.737 | 4 | 1991.924 | 1991.924 | 0 | 0.0 | 4385 | 365.5 | 365.5 | 365.5 | 7.47 |
| | [+57.021]EAVSK. | | | | | | | | | | | | |
| | F | | | | | | | | | | | | |
| 1759 | K.ILSPPDISR.F | | 499.287 | 2 | 997.568 | 997.568 | 0 | −0.1 | 4409 | 457.8 | 457.8 | 457.8 | 17.10 |
| 1759 | K.ILSPPDISR.F | | 997.567 | 1 | 997.567 | 997.568 | 0 | 0.4 | 4409 | 442.2 | 442.2 | 442.2 | 6.23 |
| 1759 | K.ILSPPDISR.F | | 499.288 | 2 | 997.568 | 997.568 | 0 | 0.1 | 4409 | 400.9 | 400.9 | 400.9 | 6.83 |
| 1759 | K.ILSPPDISR.F | | 499.288 | 2 | 997.568 | 997.568 | 0 | 0.1 | 4409 | 398.4 | 398.4 | 398.4 | 6.72 |
| 1759 | K.ILSPPDISR.F | | 499.288 | 2 | 997.568 | 997.568 | 0 | 0.1 | 4409 | 396.3 | 396.3 | 396.3 | 6.87 |
| 1782 | K.ILSPPDISRFATS | | 837.471 | 3 | 2510.397 | 2510.398 | 0 | −0.3 | 4409 | 383.4 | 383.4 | 383.4 | 7.28 |
| | LVDNSVPLLR.A | | | | | | | | | | | | |
| 1759 | K.ILSPPDISR.F | | 499.288 | 2 | 997.568 | 997.568 | 0 | 0.1 | 4409 | 382.8 | 1382.8 | 382.8 | 6.72 |
| 1759 | K.ILSPPDISR.F | | 499.287 | 2 | 997.568 | 997.568 | | 0.1 | 4409 | 332.7 | 332.7 | 332.7 | 5.33 |
| 1759 | K.ILSPPDISR.F | | 499.288 | 2 | 997.568 | 997.568 | 0 | 0.3 | 4409 | 327.7 | 327.7 | 327.7 | 15.33 |
| 1759 | K.ILSPPDISR.F | | 499.288 | 2 | 997.568 | 997.568 | 0 | 0.1 | 4409 | 309.1 | 309.1 | 309.1 | 5.24 |
| 887 | R.FATSLVDNSVPLL | | 766.427 | 2 | 1531.848 | 1531.848 | 0 | −0.3 | 4418 | 654.4 | 654.4 | 654.4 | 11.26 |
| | R.A | | | | | | | | | | | | |
| 887 | R.FATSLVDNSVPLL | | 766.428 | 2 | 1531.848 | 1531.848 | 0 | 0.0 | 4418 | 597.1 | 597.1 | 597.1 | 9.96 |
| | R.A | | | | | | | | | | | | |
| 887 | R.FATSLVDNSVPLL | | 766.427 | 2 | 1531.847 | 1531.848 | 0 | −0.3 | 4418 | 585.5 | 585.5 | 585.5 | 9.26 |
| | R.A | | | | | | | | | | | | |
| 887 | R.FATSLVDNSVPLL | | 766.427 | 2 | 1531.848 | 1531.848 | 0 | −0.3 | 4418 | 511.0 | 511.0 | 511.0 | 8.21 |
| | R.A | | | | | | | | | | | | |
| 887 | R.FATSLVDNSVPLL | | 511.287 | 3 | 1531.847 | 1531.848 | 0 | −0.4 | 4418 | 454.8 | 454.8 | 454.8 | 6.64 |
| | R.A | | | | | | | | | | | | |
| 1485 | R.FATSLVDN | N[+1] | 766.920 | 2 | 1532.832 | 1532.832 | 0 | 0.2 | 4418 | 438.4 | 438.4 | 438.4 | 17.59 |
| | [+0.984]S | | | | | | | | | | | | |
| | VPLLR.A | | | | | | | | | | | | |
| 887 | R.FATSLVDNSVPLL | | 511.288 | 3 | 1531.848 | 1531.848 | 0 | 0.2 | 4418 | 434.1 | 434.1 | 434.1 | 7.96 |
| | R.A | | | | | | | | | | | | |
| 1485 | R.FATSLVDN | N[+1] | 511.616 | 3 | 1532.832 | 1532.832 | 0 | 0.1 | 4418 | 431.6 | 431.6 | 431.6 | 7.24 |
| | [+0.984]SVPL | | | | | | | | | | | | |
| | LR.A | | | | | | | | | | | | |
| 1485 | R.FATSLVDN | N[+1] | 766.920 | 2 | 1532.833 | 1532.832 | 0 | 0.4 | 4418 | 422.3 | 422.3 | 422.3 | 8.56 |
| | [+0.984]SVPL | | | | | | | | | | | | |
| | LR.A | | | | | | | | | | | | |
| 887 | R.FATSLVDNSVPLL | | 766.428 | 2 | 1531.848 | 1531.848 | 0 | 0.0 | 4418 | 398.6 | 398.6 | 398.6 | 7.72 |
| | R.A | | | | | | | | | | | | |
| 1485 | R.FATSLVDN | N[+1] | 766.919 | 2 | 1532.831 | 1532.832 | 0 | −0.5 | 4418 | 386.6 | 386.6 | 386.6 | 7.01 |
| | [+0.984]SVPL | | | | | | | | | | | | |
| | LR.A | | | | | | | | | | | | |
| 1098 | K.NLAFSPATMAHAF | | 938.477 | 3 | 2813.417 | 2813.411 | 0 | 1.8 | 4467 | 836.3 | 836.3 | 836.3 | 14.57 |
| | LPTMPEDLLAQAR.R | | | | | | | | | | | | |
| 1098 | K.NLAFSPATMAHAF | | 938.476 | 3 | 2813.413 | 2813.411 | 0 | 0.5 | 4467 | 686.0 | 686.0 | 686.0 | 11.58 |
| | LPTMPEDLLAQAR.R | | | | | | | | | | | | |
| 1098 | K.NLAFSPATMAHAF | | 704.109 | QTQN4 | 2813.414 | 2813.411 | 0 | 0.9 | 4467 | 581.3 | 581.3 | 581.3 | 8.98 |
| | LPTMPEDLLAQAR.R | | | | | | | | | | | | |
| 941 | K.NLAFSPATMAHAF | | 990.509 | 3 | 2969.514 | 2969.512 | 0 | 0.4 | 4467 | 572.9 | 572.9 | 572.9 | 8.66 |
| | LPTMPEDLLAQARR. | | | | | | | | | | | | |
| | W | | | | | | | | | | | | |
| 1098 | K.NLAFSPATMAHAF | | 1407.212 | 2 | 2813.416 | 2813.411 | 0 | 1.5 | 4467 | 568.7 | 568.7 | 568.7 | 9.58 |
| | LPTMPEDLLAQAR.R | | | | | | | | | | | | |
| 941 | K.NLAFSPATMAHAF | | 743.135 | 4 | 2969.516 | 2969.512 | 0 | 1.2 | 4467 | 476.4 | 464.3 | 464.3 | 7.36 |
| | LPTMPEDLLAQARR. | | | | | | | | | | | | |
| | W | | | | | | | | | | | | |
| 1639 | K.NLAFSPATM | M[+16] | 943.808 | 3 | 2829.410 | 2829.406 | 0 | 1.3 | 4467 | 433.0 | 433.0 | 164.1 | 6.97 |
| | [+15.995]AH | | | | | | | | | | | | |
| | AFLPTMPE | | | | | | | | | | | | |
| | DLLAQAR.R | | | | | | | | | | | | |
| 1303 | R.DGFHLVKDKADR. | | 467.581 | 3 | 1400.728 | 1400.728 | 0 | −0.2 | 4541 | 683.4 | 683.4 | 683.4 | 9.71 |
| | T | | | | | | | | | | | | |
| 2683 | K.ADRTQTGHVLGNP | | 452.243 | 4 | 1805.948 | 1805.948 | 0 | 0.1 | 4550 | 458.5 | 412.0 | 412.0 | 7.20 |
| | QRR.D | | | | | | | | | | | | |
| 2009 | R.TQTGHVLGNPQRR | | 488.599 | 3 | 1463.783 | 1463.783 | 0 | 0.5 | 4553 | 457.8 | 429.7 | 429.7 | 7.13 |
| | .D | | | | | | | | | | | | |
| 4638 | R.TQTGHVLGNPQR. | | 436.566 | 3 | 1307.683 | 1307.682 | 0 | 1.3 | 4553 | 340.8 | 340.8 | 340.8 | 5.71 |
| | R | | | | | | | | | | | | |
| 886 | K.GFLQQHILKDLEQ | | 627.692 | 3 | 1881.061 | 1881.059 | 0 | 0.9 | 4614 | 656.1 | 656.1 | 656.1 | 11.77 |
| | LAK.M | | | | | | | | | | | | |
| 886 | K.GFLQQHILKDLEQ | | 941.033 | 2 | 1881.059 | 1881.059 | 0 | 0.0 | 4614 | 599.5 | 599.5 | 599.5 | 8.39 |
| | LAK.M | | | | | | | | | | | | |
| 886 | K.GFLQQHILKDLEQ | | 471.020 | 4 | 1881.060 | 1881.059 | 0 | 0.4 | 4614 | 490.3 | 490.3 | 490.3 | 8.72 |
| | LAK.M | | | | | | | | | | | | |
| 886 | K.GFLQQHILKDLEQ | | 627.691 | 3 | 1881.059 | 1881.059 | 0 | 0.1 | 4614 | 363.7 | 363.7 | 363.7 | 7.53 |
| | LAK.M | | | | | | | | | | | | |
| 1043 | K.MLGHSADETIGVV | | 487.518 | 4 | 1947.049 | 1947.048 | 0 | 0.4 | 4630 | 743.0 | 743.0 | 743.0 | 12.96 |
| | HLVLR.R | | | | | | | | | | | | |
| 1043 | K.MLGHSADETIGVV | | 649.688 | 3 | 1947.049 | 1947.048 | 0 | 0.7 | 4630 | 708.8 | 708.8 | 708.8 | 12.96 |
| | HLVLR.R | | | | | | | | | | | | |
| 819 | K.MLGHSADETIGVV | | 526.543 | 4 | 2103.150 | 2103.149 | 0 | 0.2 | 4630 | 617.1 | 617.1 | 617.1 | 10.77 |
| | HLVLRR.L | | | | | | | | | | | | |
| 819 | K.MLGHSADETIGVV | | 421.436 | 5 | 2103.149 | 2103.149 | 0 | 0.0 | 4630 | 561.1 | 561.1 | 561.1 | 9.77 |
| | HLVLRR.L | | | | | | | | | | | | |
| 1043 | K.MLGHSADETIGVV | | 487.518 | 4 | 1947.048 | 1947.048 | 0 | 0.0 | 4630 | 551.6 | 551.6 | 551.6 | 9.12 |
| | HLVLR.R | | | | | | | | | | | | |
| 1625 | K.M[+15.995]LGH | M[+16] | 424.635 | 5 | 2119.145 | 2119.144 | 0 | 0.3 | 4630 | 508.9 | 508.9 | 508.9 | 8.72 |
| | SADETIGVVHLVLRR | | | | | | | | | | | | |
| | .L | | | | | | | | | | | | |
| 819 | K.MLGHSADETIGVV | | 526.543 | 4 | 2103.150 | 2103.149 | 0 | 0.2 | 4630 | 417.2 | 417.2 | 417.2 | 8.37 |
| | HLVLRR.L | | | | | | | | | | | | |
| 924 | R.RLLQEQHQLSSR. | | 498.943 | 3 | 1494.814 | 1494.814 | 0 | 0.2 | 4648 | 484.6 | 484.6 | 484.6 | 8.54 |
| | R | | | | | | | | | | | | |
| 1824 | R.LLQEQHQLSSR.R | | 446.909 | 3 | 1338.712 | 1338.712 | 0 | 0.0 | 4649 | 411.5 | 411.5 | 411.5 | 7.22 |
| 1111 | R.RLLNFDTELSTKE | | 618.324 | 3 | 1852.959 | 1852.959 | 0 | 0.0 | 4660 | 755.7 | 755.7 | 755.7 | 13.77 |
| | MR.N | | | | | | | | | | | | |
| 1111 | R.RLLNFDTELSTKE | | 463.995 | 4 | 1852.958 | 1852.959 | 0 | 0.4 | 4660 | 612.9 | 612.9 | 612.9 | 10.06 |
| | MR.N | | | | | | | | | | | | |
| 906 | R.RLLNFDTELSTK. | | 718.891 | 2 | 1436.775 | 1436.774 | 0 | 0.1 | 4660 | 599.6 | 599.6 | 599.6 | 9.59 |
| | E | | | | | | | | | | | | |
| 1111 | R.RLLNFDTELSTKE | | 463.995 | 4 | 1852.959 | 1852.959 | 0 | 0.2 | 4660 | 590.6 | 590.6 | 590.6 | 9.77 |
| | MR.N | | | | | | | | | | | | |
| 4639 | R.RLLNFDTELSTKE | M[+16] | 623.656 | 3 | 1868.954 | 1868.954 | 0 | 0.2 | 4660 | 551.9 | 1551.9 | 551.9 | 8.93 |
| | M[+15.995]R.N | | | | | | | | | | | | |
| 906 | R.RLLNFDTELSTK. | | 718.891 | 2 | 1436.775 | 1436.774 | 0 | 0.2 | 4660 | 545.1 | 545.1 | 545.1 | 8.02 |
| | E | | | | | | | | | | | | |
| 906 | R.RLLNFDTELSTK. | | 479.596 | 3 | 1436.774 | 1436.774 | 0 | −0.3 | 4660 | 509.6 | 509.6 | 509.6 | 7.83 |
| | E | | | | | | | | | | | | |
| 906 | R.RLLNFDTELSTK. | | 479.596 | 3 | 1436.774 | 1436.774 | 0 | 0.2 | 4660 | 501.4 | 501.4 | 501.4 | 8.54 |
| | E | | | | | | | | | | | | |
| 906 | R.RLLNFDTELSTK. | | 479.596 | 3 | 1436.774 | 1436.774 | 0 | 0.1 | 4660 | 446.7 | 446.7 | 446.7 | 7.57 |
| | E | | | | | | | | | | | | |
| 906 | R.RLLNFDTELSTK. | | 479.596 | 3 | 1436.774 | 1436.774 | 0 | −0.3 | 4660 | 424.1 | 419.5 | 419.5 | 6.75 |
| | E | | | | | | | | | | | | |
| 906 | R.RLLNFDTELSTK. | | 479.596 | 3 | 1436.774 | 1436.774 | 0 | −0.1 | 4660 | 412.0 | 412.0 | 412.0 | 6.99 |
| | E | | | | | | | | | | | | |
| 906 | R.RLLNFDTELSTK. | | 479.596 | 3 | 1436.774 | 1436.774 | 0 | 0.2 | 4660 | 399.6 | 399.6 | 399.6 | 6.87 |
| | E | | | | | | | | | | | | |
| 906 | R.RLLNFDTELSTK. | | 479.596 | 3 | 1436.774 | 1436.774 | 0 | 0.0 | 4660 | 378.3 | 378.3 | 378.3 | 7.10 |
| | E | | | | | | | | | | | | |
| 906 | R.RLLNFDTELSTK. | | 718.891 | 2 | 1436.775 | 1436.774 | 0 | 0.2 | 4660 | 324.8 | 324.8 | 324.8 | 5.33 |
| | E | | | | | | | | | | | | |
| 1111 | R.RLLNFDTELSTKE | | 618.324 | 3 | 1852.958 | 1852.959 | 0 | −0.4 | 4660 | 312.1 | 312.1 | 312.1 | 4.87 |
| | MR.N | | | | | | | | | | | | |
| 1533 | R.LLNFDTELSTKEM | M[+16] | 571.622 | 3 | 1712.853 | 1712.852 | 0 | 0.1 | 4661 | 559.8 | 559.8 | 559.8 | 8.20 |
| | [+15.995]R.N | | | | | | | | | | | | |
| 821 | R.LLNFDTELSTK.E | | 640.840 | 2 | 1280.673 | 1280.673 | 0 | 0.0 | 4661 | 540.0 | 1539.2 | 539.2 | 8.02 |
| 821 | R.LLNFDTELSTK.E | | 640.840 | 2 | 1280.674 | 1280.673 | 0 | 0.2 | 4661 | 526.2 | 483.0 | 483.0 | 8.02 |
| 1533 | R.LLNFDTELSTKEM | M[+16] | 856.930 | 2 | 1712.852 | 1712.852 | 0 | −0.2 | 4661 | 517.6 | 517.6 | 517.6 | 8.72 |
| | [+15.995]R.N | | | | | | | | | | | | |
| 821 | R.LLNFDTELSTK.E | | 640.841 | 2 | 1280.674 | 1280.673 | 0 | 0.4 | 4661 | 503.3 | 503.3 | 503.3 | 7.57 |
| 873 | R.LLNFDTELSTKEM | | 566.291 | 3 | 1696.857 | 1696.857 | 0 | −0.2 | 4661 | 500.2 | 500.2 | 500.2 | 8.72 |
| | R.N | | | | | | | | | | | | |
| 821 | R.LLNFDTELSTK.E | | 640.840 | 2 | 1280.673 | 1280.673 | 0 | 0.0 | 4661 | 481.4 | 349.9 | 349.9 | 6.29 |
| 821 | R.LLNFDTELSTK.E | | 640.841 | 2 | 1280.674 | 1280.673 | 0 | 0.6 | 4661 | 474.2 | 474.2 | 474.2 | 7.33 |
| 821 | R.LLNFDTELSTK.E | | 640.840 | 2 | 1280.673 | 1280.673 | 0 | −0.2 | 4661 | 457.6 | 332.5 | 332.5 | 6.04 |
| 821 | R.LLNFDTELSTK.E | | 640.840 | 2 | 1280.673 | 1280.673 | 0 | 0.0 | 4661 | 456.9 | 456.9 | 456.9 | 7.57 |
| 1533 | R.LLNFDTELSTKEM | M[+16] | 571.622 | 3 | 1712.853 | 1712.852 | 0 | 0.2 | 4661 | 407.6 | 407.6 | 407.6 | 7.17 |
| | [+15.995]R.N | | | | | | | | | | | | |
| 821 | R.LLNFDTELSTK.E | | 640.841 | 2 | 1280.674 | 1280.673 | 0 | 0.4 | 4661 | 364.2 | 360.1 | 360.1 | 6.87 |
| 821 | R.LLNFDTELSTK.E | | 640.836 | 2 | 1280.665 | 1280.673 | 0 | −6.8 | 4661 | 342.1 | 342.1 | 342.1 | 4.08 |
| 821 | R.LLNFDTELSTK.E | | 640.841 | 2 | 1280.674 | 1280.673 | 0 | 0.8 | 4661 | 308.6 | 249.1 | 249.1 | 5.54 |
| 1321 | R.NNWEKEIAAVISP | | 779.069 | 3 | 2335.193 | 2335.193 | 0 | 0.1 | 4675 | 670.4 | 670.4 | 670.4 | 11.77 |
| | ELEHLDK.T | | | | | | | | | | | | |
| 1014 | R.NNWEKEIAAVISP | | 758.992 | 5 | 3790.932 | 3790.932 | 0 | −0.1 | 4675 | 610.7 | 610.7 | 610.7 | 10.08 |
| | ELEHLDKTLPTMNNL | | | | | | | | | | | | |
| | ISQDK.R | | | | | | | | | | | | |
| 1498 | R.N[+0.984]NWEK | N[+1] | 584.800 | 4 | 2336.178 | 2336.177 | 0 | 0.4 | 4675 | 541.0 | 541.0 | 0.0 | 8.93 |
| | EIAAVISPELEHLDK | | | | | | | | | | | | |
| | .T | | | | | | | | | | | | |
| 1498 | R.N[+0.984]NWEK | N[+1] | 779.397 | 3 | 2336.178 | 2336.177 | 0 | 0.3 | 4675 | 487.0 | 487.0 | 10.8 | 7.75 |
| | EIAAVISPELEHLDK | | | | | | | | | | | | |
| | .T | | | | | | | | | | | | |
| 1055 | K.EIAAVISPELEHL | | 1092.581 | 3 | 3275.730 | 3275.730 | 0 | −0.2 | 4680 | 628.4 | 628.4 | 628.4 | 8.70 |
| | DKTLPTMNNLISQDK | | | | | | | | | | | | |
| | R.I | | | | | | | | | | | | |
| 1597 | K.EIAAVISPELEHL | M[+16] | 823.937 | 4 | 3292.726 | 3291.725 | 1 | 0.9 | 4680 | 560.6 | 413.9 | 349.3 | 7.40 |
| | DKTLPTM | | | | | | | | | | | | |
| | [+15.995]NNLIS | | | | | | | | | | | | |
| | QDKR.I | | | | | | | | | | | | |
| 933 | K.EIAAVISPELEHL | | 832.449 | 2 | 1663.890 | 1663.890 | 0 | 0.0 | 4680 | 504.7 | 504.7 | 504.7 | 18.91 |
| | DK.T | | | | | | | | | | | | |
| 1055 | K.EIAAVISPELEHL | | 819.688 | 4 | 3275.730 | 3275.730 | 0 | −0.2 | 4680 | 500.4 | 500.4 | 500.4 | 8.03 |
| | DKTLPTMNNLISQDK | | | | | | | | | | | | |
| | R.I | | | | | | | | | | | | |
| 1055 | K.EIAAVISPELEHL | | 1093.248 | 3 | 3277.730 | 3275.730 | 2 | −2.1 | 4680 | 393.1 | 393.1 | 148.5 | 4.27 |
| | DKTLPTMNNLISQDK | | | | | | | | | | | | |
| | R.I | | | | | | | | | | | | |
| 933 | K.EIAAVISPELEHL | | 555.636 | 3 | 1664.893 | 1663.890 | 1 | −0.6 | 4680 | 388.8 | 388.8 | 388.8 | 6.77 |
| | DK.T | | | | | | | | | | | | |
| 1597 | K.EIAAVISPELEHL | M[+16] | 549.623 | 6 | 3292.701 | 3291.725 | 1 | −8.6 | 4680 | 313.1 | 313.1 | 186.0 | 2.71 |
| | DKTLPTM | | | | | | | | | | | | |
| | [+15.995]NN | | | | | | | | | | | | |
| | LISQDKR.I | | | | | | | | | | | | |
| 882 | K.TLPTMNNLISQDK | | 815.933 | 2 | 1630.859 | 1630.858 | 0 | 0.5 | 4695 | 521.1 | 521.1 | 521.1 | 8.20 |
| | R.I | | | | | | | | | | | | |
| 882 | K.TLPTMNNLISQDK | | 815.933 | | 1630.859 | 1630.858 | 0 | 0.3 | 4695 | 496.4 | 496.4 | 496.4 | 7.99 |
| | R.I | | | | | | | | | | | | |
| 1574 | K.TLPTM | M[+16] | 549.623 | 3 | 1646.854 | 1646.853 | 0 | 0.5 | 4695 | 434.5 | 434.5 | 434.5 | 7.40 |
| | [+15.995]N | | | | | | | | | | | | |
| | NLISQDKR.I | | | | | | | | | | | | |
| 1574 | K.TLPTM | M[+16] | 549.622 | 3 | 1646.853 | 1646.853 | 0 | −0.2 | 4695 | 406.1 | 406.1 | 406.1 | 7.40 |
| | [+15.995] | | | | | | | | | | | | |
| | NNLISQDKR.I | | | | | | | | | | | | |
| 882 | K.TLPTMNNLISQDK | | 544.291 | 3 | 1630.859 | 1630.858 | 0 | 0.3 | 4695 | 307.4 | 307.4 | 307.4 | 5.58 |
| | R.I | | | | | | | | | | | | |
| 1838 | R.ISSNPVAK.I | | 408.235 | 2 | 815.462 | 815.462 | 0 | 0.0 | 4709 | 317.0 | 317.0 | 317.0 | 5.07 |
| 1172 | K.JIYGDPVTFLPHL | | 869.487 | | 1737.967 | 1737.969 | 0 | −0.8 | 4717 | 745.9 | 745.9 | 745.9 | 12.26 |
| | PR.K | | | | | | | | | | | | |
| 1172 | K.IIYGDPVTFLPHL | | 579.994 | 3 | 1737.968 | 1737.969 | 0 | −0.2 | 4717 | 691.8 | 691.8 | 691.8 | 11.96 |
| | PR.K | | | | | | | | | | | | |
| 846 | K.IIYGDPVTFLPHL | | 467.271 | 4 | 1866.064 | 1866.064 | 0 | 0.0 | 4717 | 639.1 | 639.1 | 639.1 | 10.77 |
| | PRK.S | | | | | | | | | | | | |
| 1172 | K.JIYGDPVTFLPHL | | 579.993 | 3 | 1737.965 | 1737.969 | 0 | −1.9 | 4717 | 621.9 | 621.9 | 621.9 | 10.26 |
| | PR.K | | | | | | | | | | | | |
| 1172 | K.IIYGDPVTFLPHL | | 579.994 | 3 | 1737.968 | 1737.969 | 0 | −0.7 | 4717 | 613.2 | 613.2 | 613.2 | 10.26 |
| | PR.K | | | | | | | | | | | | |
| 846 | K.IIYGDPVTFLPHL | | 467.272 | 4 | 1866.064 | 1866.064 | 0 | 0.4 | 4717 | 608.0 | 608.0 | 608.0 | 10.77 |
| | PRK.S | | | | | | | | | | | | |
| 846 | K.IIYGDPVTFLPHL | | 467.271 | 4 | 1866.064 | 1866.064 | 0 | 0.0 | 4717 | 558.0 | 558.0 | 558.0 | 18.93 |
| | PRK.S | | | | | | | | | | | | |
| 846 | K.IIYGDPVTFLPHL | | 467.271 | 4 | 1866.064 | 1866.064 | 0 | −0.1 | 4717 | 509.1 | 463.9 | 463.9 | 8.72 |
| | PRK.S | | | | | | | | | | | | |
| 846 | K.IIYGDPVTFLPHL | | 622.693 | 3 | 1866.065 | 1866.064 | 0 | 0.6 | 4717 | 475.5 | 475.5 | 475.5 | 18.48 |
| | PRK.S | | | | | | | | | | | | |
| 1172 | K.IIYGDPVTFLPHL | | 579.994 | 3 | 1737.969 | 1737.969 | 0 | 0.0 | 4717 | 435.7 | 435.7 | 435.7 | 8.56 |
| | PR.K | | | | | | | | | | | | |
| 846 | K.IIYGDPVTFLPHL | | 622.693 | 3 | 1866.064 | 1866.064 | 0 | 0.3 | 4717 | 424.3 | 424.3 | 424.3 | 7.64 |
| | PRK.S | | | | | | | | | | | | |
| 846 | K.IIYGDPVTFLPHL | | 467.271 | 4 | 1866.064 | 1866.064 | 0 | 0.0 | 4717 | 422.8 | 422.8 | 422.8 | 18.37 |
| | PRK.S | | | | | | | | | | | | |
| 2008 | R.KRITVEYLQHIVE | | 471.773 | 4 | 1884.071 | 1884.070 | 0 | 0.2 | 4745 | 427.0 | 427.0 | 427.0 | 7.64 |
| | QK.N | | | | | | | | | | | | |
| 1159 | R.ITVEYLQHIVEQK | | 800.440 | 2 | 1599.874 | 1599.874 | 0 | −0.4 | 4747 | 679.3 | 679.3 | 679.3 | 10.88 |
| | .N | | | | | | | | | | | | |
| 1159 | R.ITVEYLQHIVEQK | | 800.441 | 2 | 1599.875 | 1599.874 | 0 | 0.4 | 4747 | 644.2 | 481.6 | 481.6 | 10.59 |
| | .N | | | | | | | | | | | | |
| 1159 | R.ITVEYLQHIVEQK | | 800.441 | 2 | 1599.874 | 1599.874 | 0 | 0.1 | 4747 | 573.2 | 550.4 | 550.4 | 9.59 |
| | .N | | | | | | | | | | | | |
| 1512 | R.ITVEYLQHIVEQK | N[+1] | 547.045 | 4 | 2185.160 | 2185.161 | 0 | 0.6 | 4747 | 486.9 | 486.9 | 472.3 | 7.70 |
| | N[+0.984]GKER.V | | | | | | | | | | | | |
| 4640 | K.NGKERVPILWHFL | | 467.270 | 4 | 1866.057 | 1865.054 | 1 | 0.2 | 4760 | 320.0 | 320.0 | 320.0 | 5.20 |
| | QK.E | | | | | | | | | | | | |
| 879 | R.VPILWHFLQKEAE | | 470.520 | 4 | 1879.060 | 1879.059 | 0 | 0.4 | 4765 | 480.5 | 480.5 | 480.5 | 8.72 |
| | LR.L | | | | | | | | | | | | |
| 863 | R.VPILWHFLQK.E | | 427.589 | 3 | 1280.752 | 1280.751 | 0 | 0.2 | 4765 | 423.1 | 423.1 | 423.1 | 7.22 |
| 4641 | R.LVKFLPEILALQR | | 513.992 | 3 | 1539.962 | 1539.962 | 0 | 0.1 | 4780 | 451.4 | 451.4 | 451.4 | 7.38 |
| | .D | | | | | | | | | | | | |
| 853 | K.FLPEILALQR.D | | 600.361 | 2 | 1199.714 | 1199.715 | 0 | 0.5 | 4783 | 495.6 | 495.6 | 495.6 | 6.86 |
| 853 | K.FLPEILALQR.D | | 600.361 | 2 | 1199.715 | 1199.715 | 0 | 0.2 | 4783 | 462.2 | 462.2 | 462.2 | 7.33 |
| 1390 | R.DLVKQFQNVQQVE | | 905.149 | 3 | 2713.432 | 2713.431 | 0 | 0.4 | 4793 | 732.7 | 732.7 | 1732.7 | 12.46 |
| | YSSIRGFLSK.H | | | | | | | | | | | | |
| 1019 | R.DLVKQFQNVQQVE | | 1091.069 | 2 | 2181.131 | 2181.130 | 0 | 0.6 | 4793 | 632.5 | 548.9 | 548.9 | 10.77 |
| | YSSIR.G | | | | | | | | | | | | |
| 1019 | R.DLVKQFQNVQQVE | | 727.715 | 3 | 2181.132 | 2181.130 | 0 | 0.8 | 4793 | 541.7 | 541.7 | 541.7 | 8.93 |
| | YSSIR.G | | | | | | | | | | | | |
| 1019 | R.DLVKQFQNVQQVE | | 727.715 | 3 | 2181.132 | 2181.130 | 0 | 0.8 | 4793 | 537.9 | 537.9 | 537.9 | 18.93 |
| | YSSIR.G | | | | | | | | | | | | |
| 818 | K.QFQNVQQVEYSSI | | 863.431 | 2 | 1725.855 | 1725.856 | 0 | −0.5 | 4797 | 676.2 | 676.2 | 676.2 | 11.26 |
| | R.G | | | | | | | | | | | | |
| 1050 | K.QFQNVQQVEYSSI | | 753.391 | 3 | 2258.157 | 2258.156 | 0 | 0.4 | 4797 | 552.6 | 552.6 | 552.6 | 18.20 |
| | RGFLSK.H | | | | | | | | | | | | |
| 818 | K.QFQNVQQVEYSSI | | 575.957 | 3 | 1725.856 | 1725.856 | 0 | 0.1 | 4797 | 475.3 | 475.3 | 475.3 | 7.34 |
| | R.G | | | | | | | | | | | | |
| 925 | R.ITVFLSTWNK.L | | 604.837 | 2. | 1208.667 | 1208.667 | 0 | −0.3 | 4829 | 507.2 | 292.3 | 292.3 | 6.38 |
| 1720 | R.SLETNGEINLPKD | C[+57] | 1204.255 | 3 | 3610.751 | 3610.747 | 0 | 1.2 | 4842 | 695.5 | 695.5 | 695.5 | 11.39 |
| | YC[+57.021]STDL | | | | | | | | | | | | |
| | DLDTEFEILLPR.R | | | | | | | | | | | | |
| 1720 | R.SLETNGEINLPKD | C[+57] | 903.443 | 4 | 3610.752 | 3610.747 | 0 | 1.3 | 4842 | 622.6 | 575.7 | 575.7 | 8.82 |
| | YC[+57.021]STDL | | | | | | | | | | | | |
| | DLDTEFEILLPR.R | | | | | | | | | | | | |
| 1146 | R.SLETNGEINLPK. | | 657.849 | 2 | 1314.690 | 1314.690 | 0 | 0.0 | 4842 | 599.7 | 599.7 | 599.7 | 9.59 |
| | D | | | | | | | | | | | | |
| 1715 | R.GLGLC | C[+57] | 803.954 | 2 | 1606.900 | 1606.899 | 0 | 0.9 | 4875 | 496.0 | 496.0 | 496.0 | 8.91 |
| | [+57.021] | | | | | | | | | | | | |
| | ATALVSYLIR.L | | | | | | | | | | | | |
| 1715 | R.GLGLC[+57.021 | C[+57] | 536.305 | 3 | 1606.899 | 1606.899 | 0 | 0.5 | 4875 | 481.3 | 481.3 | 481.3 | 7.59 |
| | ]ATALVSYLIR.L | | | | | | | | | | | | |
| 1246 | R.LHNEIVYAVEK.L | | 657.856 | 2 | 1314.705 | 1314.705 | 0 | −0.4 | 4890 | 651.9 | 651.9 | 651.9 | 10.16 |
| 1246 | R.LHNEIVYAVEK.L | | 438.907 | 3 | 1314.705 | 1314.705 | 0 | 0.1 | 4890 | 451.2 | 451.2 | 451.2 | 7.57 |
| 1819 | R.LHNEIVYAVEKLS | | 411.485 | 4 | 1642.916 | 1642.916 | 0 | 0.1 | 4890 | 451.1 | 451.1 | 451.1 | 8.48 |
| | K.E | | | | | | | | | | | | |
| 1246 | R.LHNEIVYAVEK.L | | 438.907 | 3 | 1314.705 | 1314.705 | 0 | −0.2 | 4890 | 443.6 | 443.6 | 443.6 | 7.10 |
| 900 | R.ETVQEFDLEK.I | | 619.301 | 2 | 1237.595 | 1237.595 | 0 | 0.2 | 4946 | 511.6 | 511.6 | 511.6 | 17.81 |
| 4342 | R.ETVQEFDLEKIQR | | 817.923 | 2 | 1634.839 | 1634.838 | 0 | 0.2 | 4946 | 331.3 | 331.3 | 331.3 | 5.52 |
| | .Q | | | | | | | | | | | | |
| 1004 | R.LSLKGIPTLVYR. | | 680.422 | 2 | 1359.836 | 1359.836 | 0 | 0.2 | 4971 | 633.4 | 633.4 | 633.4 | 9.67 |
| | H | | | | | | | | | | | | |
| 1004 | R.LSLKGIPTLVYR. | | 453.950 | 3 | 1359.836 | 1359.836 | 0 | 0.1 | 4971 | 559.8 | 559.8 | 559.8 | 18.56 |
| | H | | | | | | | | | | | | |
| 1004 | R.LSLKGIPTLVYR. | | 453.950 | 3 | 1359.836 | 1359.836 | 0 | 0.1 | 4971 | 542.8 | 542.8 | 542.8 | 17.83 |
| | H | | | | | | | | | | | | |
| 1912 | K.GIPTLVYR.H | | 459.774 | 2 | 918.541 | 918.541 | 0 | 0.0 | 4975 | 370.4 | 370.4 | 370.4 | 6.72 |
| 1611 | R.HDWNYEHLFM | M[+16] | 502.236 | 4 | 2005.921 | 2005.923 | 0 | −0.6 | 4983 | 560.9 | 557.0 | 557.0 | 9.06 |
| | [+15.995] | | | | | | | | | | | | |
| | DIKNK.M | | | | | | | | | | | | |
| 966 | R.HDWNYEHLFMDIK | | 583.268 | 3 | 1747.790 | 1747.790 | 0 | 0.1 | 4983 | 543.5 | 543.5 | 543.5 | 8.02 |
| | .N | | | | | | | | | | | | |
| 966 | R.HDWNYEHLFMDIK | | 583.268 | 3 | 1747.790 | 1747.790 | 0 | 0.3 | 4983 | 510.5 | 510.5 | 510.5 | 17.81 |
| | .N | | | | | | | | | | | | |
| 1565 | R.HDWNYEHLFM | M[+16] | 588.600 | 3 | 1763.785 | 1763.785 | 0 | 0.1 | 4983 | 459.9 | 459.9 | 459.9 | 7.10 |
| | [+15.995] | | | | | | | | | | | | |
| | DIK.N | | | | | | | | | | | | |
| 966 | R.HDWNYEHLFMDIK | | 437.703 | 4 | 1747.791 | 1747.790 | 0 | 0.6 | 4983 | 402.1 | 357.8 | 357.8 | 5.93 |
| | .N | | | | | | | | | | | | |
| 966 | R.HDWNYEHLFMDIK | | 874.399 | 2 | 1747.790 | 1747.790 | 0 | 0.0 | 4983 | 369.1 | 369.1 | 369.1 | 6.69 |
| | .N | | | | | | | | | | | | |
| 1020 | K.HTIALWQFLSAHK | | 570.317 | 4 | 2278.246 | 2278.246 | 0 | 0.1 | 5074 | 695.9 | 695.9 | 695.9 | 11.77 |
| | SEQLLR.L | | | | | | | | | | | | |
| 1020 | K.HTIALWQFLSAHK | | 570.317 | 4 | 2278.246 | 2278.246 | 0 | 0.0 | 5074 | 684.3 | 1684.3 | 684.3 | 11.77 |
| | SEQLLR.L | | | | | | | | | | | | |
| 1020 | K.HTIALWQFLSAHK | | 570.317 | 4 | 2278.246 | 2278.246 | 0 | 0.0 | 5074 | 592.4 | 592.4 | 592.4 | 9.77 |
| | SEQLLR.L | | | | | | | | | | | | |
| 1020 | K.HTIALWQFLSAHK | | 570.317 | 4 | 2278.244 | 2278.246 | 0 | 0.5 | 5074 | 528.3 | 528.3 | 528.3 | 8.23 |
| | SEQLLR.L | | | | | | | | | | | | |
| 2095 | K.HTIALWQFLSAHK | | 517.952 | 3 | 1551.842 | 1551.843 | 0 | 0.4 | 5074 | 443.5 | 1443.5 | 443.5 | 6.86 |
| | .S | | | | | | | | | | | | |
| 2095 | K.HTIALWQFLSAHK | | 517.953 | 3 | 1551.843 | 1551.843 | 0 | −0.2 | 5074 | 400.1 | 400.1 | 400.1 | 7.22 |
| | .S | | | | | | | | | | | | |
| 903 | R.LHKEPFGEISSR. | | 467.250 | 3 | 1399.735 | 1399.733 | 0 | 1.9 | 5093 | 651.8 | 651.8 | 651.8 | 10.67 |
| | Y | | | | | | | | | | | | |
| 903 | R.LHKEPFGEISSR. | | 467.249 | 3 | 1399.733 | 1399.733 | 0 | 0.3 | 5093 | 637.8 | 637.8 | 637.8 | 9.67 |
| | Y | | | | | | | | | | | | |
| 903 | R.LHKEPFGEISSR. | | 467.250 | 3 | 1399.735 | 1399.733 | 0 | 1.8 | 5093 | 600.8 | 600.8 | 600.8 | 10.40 |
| | Y | | | | | | | | | | | | |
| 903 | R.LHKEPFGEISSR. | | 700.370 | 2 | 1399.733 | 1399.733 | 0 | 0.1 | 5093 | 591.3 | 591.3 | 591.3 | 7.29 |
| | Y | | | | | | | | | | | | |
| 903 | R.LHKEPFGEISSR. | | 467.249 | 3 | 1399.733 | 1399.733 | 0 | 0.4 | 5093 | 572.5 | 572.5 | 572.5 | 8.67 |
| | Y | | | | | | | | | | | | |
| 903 | R.LHKEPFGEISSR. | | 467.249 | 3 | 1399.733 | 1399.733 | 0 | 0.1 | 5093 | 555.2 | 555.2 | 555.2 | 7.83 |
| | Y | | | | | | | | | | | | |
| 903 | R.LHKEPFGEISSR. | | 467.249 | 3 | 1399.733 | 1399.733 | 0 | 0.1 | 5093 | 548.6 | 548.6 | 548.6 | 7.83 |
| | Y | | | | | | | | | | | | |
| 903 | R.LHKEPFGEISSR. | | 467.249 | 3 | 1399.733 | 1399.733 | 0 | −0.2 | 5093 | 513.1 | 513.1 | 513.1 | 17.38 |
| | Y | | | | | | | | | | | | |
| 903 | R.LHKEPFGEISSR. | | 467.250 | 3 | 1399.734 | 1399.733 | 0 | 1.0 | 5093 | 503.8 | 503.8 | 503.8 | 7.62 |
| | Y | | | | | | | | | | | | |
| 903 | R.LHKEPFGEISSR. | | 467.581 | 3 | 1400.729 | 1399.733 | 1 | 5.2 | 5093 | 462.8 | 393.0 | 393.0 | 4.33 |
| | Y | | | | | | | | | | | | |
| 903 | R.LHKEPFGEISSR. | | 467.249 | 3 | 1399.733 | 1399.733 | 0 | 0.2 | 5093 | 455.0 | 455.0 | 455.0 | 7.13 |
| | Y | | | | | | | | | | | | |
| 903 | R.LHKEPFGEISSR. | | 467.249 | 3 | 1399.732 | 1399.733 | 0 | −0.5 | 5093 | 429.0 | 429.0 | 429.0 | 6.32 |
| | Y | | | | | | | | | | | | |
| 903 | R.LHKEPFGEISSR. | | 467.249 | 3 | 1399.733 | 1399.733 | 0 | 0.4 | 5093 | 388.8 | 286.3 | 286.3 | 5.48 |
| | Y | | | | | | | | | | | | |
| 903 | R.LHKEPFGEISSR. | | 467.253 | 3 | 1399.743 | 1399.733 | 0 | 7.4 | 5093 | 344.7 | 287.7 | 287.7 | 3.63 |
| | Y | | | | | | | | | | | | |
| 903 | R.LHKEPFGEISSR. | | 467.249 | 3 | 1399.733 | 1399.733 | 0 | 0.4 | 5093 | 312.0 | 312.0 | 312.0 | 5.21 |
| | Y | | | | | | | | | | | | |
| 2609 | R.YKADLSPENAK.L | | 618.317 | 2 | 1235.627 | 1235.627 | 0 | 0.1 | 5105 | 456.6 | 456.6 | 456.6 | 6.90 |
| 1450 | K.LLSTFLNQTGLDA | | 958.201 | 3 | 2872.590 | 2872.589 | 0 | 0.1 | 5116 | 857.1 | 857.1 | 857.1 | 15.65 |
| | FLLELHEMIILK.L | | | | | | | | | | | | |
| 3259 | K.LLSTFLNQTGLDA | M[+16] | 722.901 | 4 | 2888.583 | 2888.584 | 0 | −0.4 | 5116 | 776.4 | 776.4 | 776.4 | 12.27 |
| | FLLELHEM | | | | | | | | | | | | |
| | [+15.995] | | | | | | | | | | | | |
| | IILK.L | | | | | | | | | | | | |
| 3259 | K.LLSTFLNQTGLDA | M[+16] | 963.534 | 3 | 2888.587 | 2888.584 | 0 | 0.9 | 5116 | 613.2 | 613.2 | 613.2 | 10.58 |
| | FLLELHEM | | | | | | | | | | | | |
| | [+15.995]IIL | | | | | | | | | | | | |
| | K.L | | | | | | | | | | | | |
| 2126 | R.FRPQWSLR.D | | 545.301 | 2 | 1089.595 | 1089.595 | 0 | −0.3 | 5152 | 429.2 | 429.2 | 429.2 | 6.12 |
| 1786 | R.FRPQWSLRDTLVS | | 752.724 | 3 | 2256.159 | 2256.159 | 0 | −0.3 | 5152 | 364.8 | 364.8 | 364.8 | 6.34 |
| | YMQTK.E | | | | | | | | | | | | |
| 1786 | R.FRPQWSLRDTLVS | | 564.795 | 4 | 2256.159 | 2256.159 | 0 | 0.2 | 5152 | 324.3 | 324.3 | 324.3 | 5.66 |
| | YMQTK.E | | | | | | | | | | | | |
| 1540 | R.DTLVSYM | M[+16] | 601.292 | 2 | 1201.577 | 1201.577 | 0 | −0.3 | 5160 | 508.8 | 508.8 | 508.8 | 7.81 |
| | [+15.995] | | | | | | | | | | | | |
| | QTK.E | | | | | | | | | | | | |
| 861 | R.DTLVSYMQTK.E | | 593.295 | 2 | 1185.582 | 1185.582 | 0 | 0.2 | 5160 | 495.0 | 495.0 | 495.0 | 17.81 |
| 861 | R.DTLVSYMQTK.E | | 593.295 | 2 | 1185.583 | 1185.582 | 0 | 0.5 | 5160 | 425.8 | 425.8 | 425.8 | 7.46 |
| 861 | R.DTLVSYMQTK.E | | 593.295 | 2 | 1185.582 | 1185.582 | 0 | 0.1 | 5160 | 412.1 | 412.1 | 412.1 | 6.99 |
| 861 | R.DTLVSYMQTK.E | | 593.295 | 2 | 1185.582 | 1185.582 | 0 | 0.4 | 5160 | 383.2 | 383.2 | 383.2 | 7.10 |
| 861 | R.DTLVSYMQTK.E | | 593.295 | 2 | 1185.582 | 1185.582 | 0 | 0.0 | 5160 | 354.7 | 354.7 | 354.7 | 15.33 |
| 1540 | R.DTLVSYM | M[+16] | 601.292 | 2 | 1201.577 | 1201.577 | 0 | 0.1 | 5160 | 315.5 | 315.5 | 315.5 | 5.40 |
| | [+15.995] | | | | | | | | | | | | |
| | QTK.E | | | | | | | | | | | | |
| 4642 | R.DT[+79.966]LV | C[+57], | 1413.672 | 3 | 4239.002 | 4238.999 | 0 | 0.8 | 5160 | 303.2 | 303.2 | 5.7 | 4.52 |
| | SYMQTKESEILPEMA | T[+80] | | | | | | | | | | | |
| | SQFPEEILLASC | | | | | | | | | | | | |
| | [+57.021]VSV | | | | | | | | | | | | |
| | WK.T | | | | | | | | | | | | |
| 4643 | K.ESEILPEMASQFP | C[+57] | 895.213 | 4 | 3577.829 | 3575.838 | 2 | −4.2 | 5170 | 318.9 | 231.4 | 231.4 | 2.89 |
| | EEILLASC | | | | | | | | | | | | |
| | [+57.021]VSVWK | | | | | | | | | | | | |
| | TAAVLK.W | | | | | | | | | | | | |
| 1144 | K.DNTVPLKLIALLA | | 1104.244 | 3 | 3310.717 | 3310.710 | 0 | 2.2 | 5226 | 800.7 | 790.3 | 790.3 | 14.39 |
| | NGEFHSGEQLGETLG | | | | | | | | | | | | |
| | MSR.A | | | | | | | | | | | | |
| 1575 | K.DNTVPLKLIALLA | M[+16] | 832.433 | 4 | 3326.711 | 3326.705 | 0 | 1.8 | 5226 | 760.5 | 760.5 | 760.5 | 13.39 |
| | NGEFHSGEQLGETLG | | | | | | | | | | | | |
| | M[+15.995]SR.A | | | | | | | | | | | | |
| 1144 | K.DNTVPLKLIALLA | | 662.949 | 5 | 3310.714 | 3310.710 | 0 | 1.3 | 5226 | 586.0 | 514.3 | 514.3 | 8.79 |
| | NGEFHSGEQLGETLG | | | | | | | | | | | | |
| | MSR.A | | | | | | | | | | | | |
| 1575 | K.DNTVPLKLIALLA | M[+16] | 832.683 | 1 | 3327.708 | 3326.705 | 1 | −0.1 | 5226 | 555.5 | 478.4 | 216.5 | 7.82 |
| | NGEFHSGEQLGETLG | | | | | | | | | | | | |
| | M[+15.995]SR.A | | | | | | | | | | | | |
| 1575 | K.DNTVPLKLIALLA | M[+16] | 1109.575 | 3 | 3326.710 | 3326.705 | 0 | 1.6 | 5226 | 523.8 | 523.8 | 523.8 | 7.58 |
| | NGEFHSGEQLGETLG | | | | | | | | | | | | |
| | M[+15.995]SR.A | | | | | | | | | | | | |
| 874 | K.LIALLANGEFHSG | | 848.764 | 3 | 2544.278 | 2543.292 | 1 | 7.1 | 5233 | 676.5 | 542.5 | 79.9 | 10.71 |
| | EQLGETLGMSR.A | | | | | | | | | | | | |
| 874 | K.LIALLANGEFHSG | | 848.435 | 3 | 2543.291 | 2543.292 | 0 | 0.3 | 5233 | 617.0 | 553.1 | 553.1 | 10.96 |
| | EQLGETLGMSR.A | | | | | | | | | | | | |
| 874 | K.LIALLANGEFHSG | | 848.429 | 3 | 2543.274 | 2543.292 | 0 | −7.4 | 5233 | 612.8 | 577.3 | 577.3 | 9.71 |
| | EQLGETLGMSR.A | | | | | | | | | | | | |
| 874 | K.LIALLANGEFHSG | | 848.435 | 3 | 2543.291 | 2543.292 | 0 | 0.3 | 5233 | 597.0 | 597.0 | 597.0 | 9.96 |
| | EQLGETLGMSR.A | | | | | | | | | | | | |
| 874 | K.LIALLANGEFHSG | | 848.436 | 3 | 2543.292 | 2543.292 | 0 | −0.2 | 5233 | 593.8 | 593.8 | 593.8 | 9.96 |
| | EQLGETLGMSR.A | | | | | | | | | | | | |
| 1515 | K.LIALLAN | N[+1] | 848.763 | 3 | 2544.276 | 2544.276 | 0 | −0.2 | 5233 | 591.1 | 558.5 | 267.7 | 9.96 |
| | [+0.984]GEFH | | | | | | | | | | | | |
| | SGEQLGETLGMSR.A | | | | | | | | | | | | |
| 874 | K.LIALLANGEFHSG | | 636.830 | 4 | 2544.297 | 2543.292 | 1 | 0.6 | 5233 | 484.3 | 484.3 | 211.3 | 8.32 |
| | EQLGETLGMSR.A | | | | | | | | | | | | |
| 874 | K.LIALLANGEFHSG | | 848.429 | 3 | 2543.273 | 2543.292 | 0 | −7.7 | 5233 | 338.9 | 338.9 | 338.9 | 4.45 |
| | EQLGETLGMSR.A | | | | | | | | | | | | |
| 852 | R.AAINKHIQTLR.D | | 422.255 | 3 | 1264.751 | 1264.748 | 0 | 1.6 | 5257 | 486.9 | 486.9 | 486.9 | 8.35 |
| 852 | R.AAINKHIQTLR.D | | 422.255 | 3 | 1264.750 | 1264.748 | 0 | 0.9 | 5257 | 436.6 | 436.6 | 436.6 | 8.00 |
| 1796 | R.AAINKHIQTLRDW | | 642.104 | 4 | 2565.394 | 2565.394 | 0 | 0.3 | 5257 | 408.1 | 408.1 | 408.1 | 7.09 |
| | GVDVFTVPGK.G | | | | | | | | | | | | |
| 1796 | R.AAINKHIQTLRDW | | 642.104 | 4 | 2565.394 | 2565.394 | 0 | 0.3 | 5257 | 351.3 | 351.3 | 351.3 | 5.35 |
| | GVDVFTVPGK.G | | | | | | | | | | | | |
| 826 | K.HIQTLRDWGVDVF | | 890.987 | 4 | 3560.927 | 3560.926 | 0 | 0.3 | 5262 | 717.3 | 717.3 | 717.3 | 12.08 |
| | TVPGKGYSLPEPIPL | | | | | | | | | | | | |
| | LNAK.Q | | | | | | | | | | | | |
| 826 | K.HIQTLRDWGVDVF | | 1187.648 | 3 | 3560.929 | 3560.926 | 0 | 0.8 | 5262 | 677.9 | 677.9 | 677.9 | 9.70 |
| | TVPGKGYSLPEPIPL | | | | | | | | | | | | |
| | LNAK.Q | | | | | | | | | | | | |
| 826 | K.HIQTLRDWGVDVF | | 890.987 | 4 | 3560.927 | 3560.926 | 0 | 0.1 | 5262 | 662.3 | 662.3 | 662.3 | 11.08 |
| | TVPGKGYSLPEPIPL | | | | | | | | | | | | |
| | LNAK.Q | | | | | | | | | | | | |
| 826 | K.HIQTLRDWGVDVF | | 1187.982 | 3 | 3561.930 | 3560.926 | 1 | 0.1 | 5262 | 596.9 | 596.9 | 517.9 | 7.70 |
| | TVPGKGYSLPEPIPL | | | | | | | | | | | | |
| | LNAK.Q | | | | | | | | | | | | |
| 826 | K.HIQTLRDWGVDVF | | 712.991 | 5 | 3560.927 | 3560.926 | 0 | 0.1 | 5262 | 507.7 | 507.7 | 507.7 | 8.03 |
| | TVPGKGYSLPEPIPL | | | | | | | | | | | | |
| | LNAK.Q | | | | | | | | | | | | |
| 826 | K.HIQTLRDWGVDVF | | 594.328 | 6 | 3560.930 | 3560.926 | 0 | 0.9 | 5262 | 457.0 | 457.0 | 457.0 | 7.19 |
| | TVPGKGYSLPEPIPL | | | | | | | | | | | | |
| | LNAK.Q | | | | | | | | | | | | |
| 826 | K.HIQTLRDWGVDVF | | 712.991 | 5 | 3560.928 | 3560.926 | 0 | 0.4 | 5262 | 414.4 | 414.4 | 414.4 | 6.47 |
| | TVPGKGYSLPEPIPL | | | | | | | | | | | | |
| | LNAK.Q | | | | | | | | | | | | |
| 1810 | K.HIQTLRDWGVDVF | | 690.037 | 3 | 2068.098 | 2068.097 | 0 | 0.2 | 5262 | 413.4 | 413.4 | 413.4 | 7.40 |
| | TVPGK.G | | | | | | | | | | | | |
| 826 | K.HIQTLRDWGVDVF | | 712.991 | 5 | 3560.926 | 3560.926 | 0 | −0.1 | 5262 | 391.5 | 391.5 | 391.5 | 6.83 |
| | TVPGKGYSLPEPIPL | | | | | | | | | | | | |
| | LNAK.Q | | | | | | | | | | | | |
| 3102 | K.HIQTLRDWGVDVF | N[+1] | 713.188 | 5 | 3561.909 | 3561.910 | 0 | −0.3 | 5262 | 388.1 | 388.1 | 87.7 | 6.59 |
| | TVPGKGYSLPEPIPL | | | | | | | | | | | | |
| | LN[+0.984]AK.Q | | | | | | | | | | | | |
| 1810 | K.HIQTLRDWGVDVF | | 517.780 | 4 | 2068.098 | 2068.097 | 0 | 0.2 | 5262 | 357.1 | 357.1 | 357.1 | 5.66 |
| | TVPGK.G | | | | | | | | | | | | |
| 826 | K.HIQTLRDWGVDVF | | 712.991 | 5 | 3560.926 | 3560.926 | 0 | 0.0 | 5262 | 347.2 | 347.2 | 347.2 | 6.17 |
| | TVPGKGYSLPEPIPL | | | | | | | | | | | | |
| | LNAK.Q | | | | | | | | | | | | |
| 1109 | R.DWGVDVFTVPGKG | | 938.169 | 3 | 2812.492 | 2812.492 | 0 | 0.2 | 5268 | 925.3 | 925.3 | 925.3 | 30.00 |
| | YSLPEPIPLLNAK.Q | | | | | | | | | | | | |
| 1510 | R.DWGVDVFTVPGKG | N[+1] | 938.498 | 3 | 2813.480 | 2813.476 | 0 | 1.2 | 5268 | 731.1 | 731.1 | 460.2 | 12.39 |
| | YSLPEPIPLLN | | | | | | | | | | | | |
| | [+0.984] | | | | | | | | | | | | |
| | AK.Q | | | | | | | | | | | | |
| 1109 | R.DWGVDVFTVPGKG | | 1406.751 | 2 | 2812.495 | 2812.492 | 0 | 1.1 | 5268 | 728.5 | 728.5 | 728.5 | 12.39 |
| | YSLPEPIPLLNAK.Q | | | | | | | | | | | | |
| 1109 | R.DWGVDVFTVPGKG | | 703.879 | 4 | 2812.493 | 2812.492 | 0 | 0.3 | 5268 | 617.3 | 617.3 | 617.3 | 9.79 |
| | YSLPEPIPLLNAK.Q | | | | | | | | | | | | |
| 1109 | R.DWGVDVFTVPGKG | | 938.169 | 3 | 2812.492 | 2812.492 | 0 | 0.1 | 5268 | 541.1 | 541.1 | 541.1 | 7.82 |
| | YSLPEPIPLLNAK.Q | | | | | | | | | | | | |
| 931 | R.DWGVDVFTVPGK. | | 660.336 | 2 | 1319.664 | 1319.663 | 0 | 0.9 | 5268 | 532.0 | 532.0 | 532.0 | 7.78 |
| | G | | | | | | | | | | | | |
| 931 | R.DWGVDVFTVPGK. | | 660.335 | 2 | 1319.663 | 1319.663 | 0 | 0.2 | 5268 | 445.4 | 445.4 | 445.4 | 7.33 |
| | G | | | | | | | | | | | | |
| 931 | R.DWGVDVFTVPGK. | | 660.335 | 2 | 1319.663 | 1319.663 | 0 | 0.2 | 5268 | 357.2 | 357.2 | 357.2 | 5.48 |
| | G | | | | | | | | | | | | |
| 950 | K.GYSLPEPIPLLNA | | 756.427 | 2 | 1511.846 | 1511.847 | 0 | 0.3 | 5280 | 688.3 | 688.3 | 688.3 | 11.26 |
| | K.Q | | | | | | | | | | | | |
| 950 | K.GYSLPEPIPLLNA | | 504.621 | 3 | 1511.847 | 1511.847 | 0 | 0.1 | 5280 | 611.0 | 611.0 | 611.0 | 10.37 |
| | K.Q | | | | | | | | | | | | |
| 950 | K.GYSLPEPIPLLNA | | 756.427 | 2 | 1511.846 | 1511.847 | 0 | −0.3 | 5280 | 606.1 | 606.1 | 606.1 | 10.26 |
| | K.Q | | | | | | | | | | | | |
| 4644 | K.GYSLPEPIPLLN | N[+1] | 756.919 | 2 | 1512.831 | 1512.831 | 0 | 0.3 | 5280 | 554.7 | 521.6 | 521.6 | 9.12 |
| | [+0.984]AK.Q | | | | | | | | | | | | |
| 950 | K.GYSLPEPIPLLNA | | 756.427 | 2 | 1511.846 | 1511.847 | 0 | 0.5 | 5280 | 544.2 | 544.2 | 544.2 | 8.42 |
| | K.Q | | | | | | | | | | | | |
| 950 | K.GYSLPEPIPLLNA | | 756.427 | 2 | 1511.847 | 1511.847 | 0 | 0.1 | 5280 | 529.1 | 529.1 | 529.1 | 9.12 |
| | K.Q | | | | | | | | | | | | |
| 950 | K.GYSLPEPIPLLNA | | 756.427 | 2 | 1511.847 | 1511.847 | 0 | 0.4 | 5280 | 513.3 | 513.3 | 513.3 | 8.91 |
| | K.Q | | | | | | | | | | | | |
| 950 | K.GYSLPEPIPLLNA | | 756.427 | 2 | 1511.847 | 1511.847 | 0 | 0.2 | 5280 | 430.4 | 430.4 | 430.4 | 8.56 |
| | K.Q | | | | | | | | | | | | |
| 950 | K.GYSLPEPIPLLNA | | 756.427 | 2 | 1511.846 | 1511.847 | 0 | 0.3 | 5280 | 377.6 | 377.6 | 377.6 | 7.01 |
| | K.Q | | | | | | | | | | | | |
| 4645 | K.GYSLPEPIPLLNA | | 1091.591 | 4 | 4363.341 | 4363.355 | 0 | 3.1 | 5280 | 369.5 | 369.5 | 369.5 | 4.56 |
| | KQILGQLDGGSVAVL | | | | | | | | | | | | |
| | PVVDSTNQYLLDR.I | | | | | | | | | | | | |
| 950 | K.GYSLPEPIPLLNA | | 756.427 | | 1511.846 | 1511.847 | 0 | 0.3 | 5280 | 309.0 | 309.0 | 309.0 | 5.29 |
| | K.Q | | | | | | | | | | | | |
| 3127 | K.QILGQLDGGSVAV | N[+1] | 1137.953 | 3 | 3411.843 | 3411.837 | 0 | 1.9 | 5294 | ##### | #### | 456.3 | 30.00 |
| | LPVVDSTN | | | | | | | | | | | | |
| | [+0.984] | | | | | | | | | | | | |
| | QYLLDRIGELK.S | | | | | | | | | | | | |
| 964 | K.QILGQLDGGSVAV | | 1137.623 | 3 | 3410.855 | 3410.853 | 0 | 0.5 | 5294 | 962.3 | 962.3 | 962.3 | 30.00 |
| | LPVVDSTNQYLLDRI | | | | | | | | | | | | |
| | GELK.S | | | | | | | | | | | | |
| 964 | K.QILGQLDGGSVAV | | 1137.624 | 3 | 3410.856 | 3410.853 | 0 | 0.9 | 5294 | 944.1 | 944.1 | 944.1 | 30.00 |
| | LPVVDSTNQYLLDRI | | | | | | | | | | | | |
| | GELK.S | | | | | | | | | | | | |
| 964 | K.QILGQLDGGSVAV | | 1137.953 | 3 | 3411.844 | 3410.853 | 1 | −3.7 | 5294 | 893.1 | 893.1 | 247.5 | 14.07 |
| | LPVVDSTNQYLLDRI | | | | | | | | | | | | |
| | GELK.S | | | | | | | | | | | | |
| 893 | K.QILGQLDGGSVAV | | 1435.766 | 2 | 2870.525 | 2870.526 | 0 | 0.2 | 5294 | 863.1 | 863.1 | 863.1 | 15.65 |
| | LPVVDSTNQYLLDR. | | | | | | | | | | | | |
| | I | | | | | | | | | | | | |
| 964 | K.QILGQLDGGSVAV | | 853.469 | 4 | 3410.853 | 340.853 | 0 | 0.1 | 5294 | 835.9 | 835.9 | 835.9 | 13.79 |
| | LPVVDSTNQYLLDRI | | | | | | | | | | | | |
| | GELK.S | | | | | | | | | | | | |
| 964 | K.QILGQLDGGSVAV | | 853.469 | 4 | 3410.854 | 340.853 | 0 | 0.2 | 5294 | 832.8 | 832.8 | 832.8 | 13.79 |
| | LPVVDSTNQYLLDRI | | | | | | | | | | | | |
| | GELK.S | | | | | | | | | | | | |
| 964 | K.QILGQLDGGSVAV | | 1137.623 | 3 | 3410.853 | 340.853 | 0 | 0.0 | 5294 | 829.1 | 829.1 | 829.1 | 14.39 |
| | LPVVDSTNQYLLDRI | | | | | | | | | | | | |
| | GELK.S | | | | | | | | | | | | |
| 964 | K.QILGQLDGGSVAV | | 853.469 | 4 | 3410.854 | 340.853 | 0 | 0.2 | 5294 | 825.1 | 825.1 | 825.1 | 13.79 |
| | LPVVDSTNQYLLDRI | | | | | | | | | | | | |
| | GELK.S | | | | | | | | | | | | |
| 3127 | K.QILGQLDGGSVAV | | 1137.956 | 3 | 3411.854 | 3411.837 | 0 | 5.1 | 5294 | 737.1 | 737.1 | 162.1 | 11.15 |
| | LPVVDSTN | | | | | | | | | | | | |
| | [+0.984] | | | | | | | | | | | | |
| | QYLLDRIGELK.SN | | | | | | | | | | | | |
| | [+1] | | | | | | | | | | | | |
| 893 | K.QILGQLDGGSVAV | | 957.513 | 3 | 2870.525 | 2870.526 | 0 | 0.3 | 5294 | 729.2 | 729.2 | 729.2 | 11.27 |
| | LPVVDSTNQYLLDR. | | | | | | | | | | | | |
| | I | | | | | | | | | | | | |
| 893 | K.QILGQLDGGSVAV | | 1435.767 | 2 | 2870.526 | 2870.526 | 0 | 0.0 | 5294 | 712.8 | 712.8 | 712.8 | 12.58 |
| | LPVVDSTNQYLLDR. | | | | | | | | | | | | |
| | I | | | | | | | | | | | | |
| 893 | K.QILGQLDGGSVAV | | 1435.768 | 2 | 2870.528 | 2870.526 | 0 | 0.8 | 5294 | 709.5 | 709.5 | 709.5 | 12.58 |
| | LPVVDSTNQYLLDR. | | | | | | | | | | | | |
| | I | | | | | | | | | | | | |
| 964 | K.QILGQLDGGSVAV | | 1137.625 | 3 | 3410.860 | 340.853 | 0 | 2.1 | 5294 | 687.8 | 687.8 | 687.8 | 11.39 |
| | LPVVDSTNQYLLDRI | | | | | | | | | | | | |
| | GELK.S | | | | | | | | | | | | |
| 964 | K.QILGQLDGGSVAV | | 1137.620 | 3 | 3410.845 | 340.853 | 0 | −2.4 | 5294 | 681.7 | 681.7 | 681.7 | 10.68 |
| | LPVVDSTNQYLLDRI | | | | | | | | | | | | |
| | GELK.S | | | | | | | | | | | | |
| 964 | K.QILGQLDGGSVAV | | 853.469 | 4 | 3410.853 | 340.853 | 0 | −0.1 | 5294 | 680.7 | 680.7 | 680.7 | 10.79 |
| | LPVVDSTNQYLLDRI | | | | | | | | | | | | |
| | GELK.S | | | | | | | | | | | | |
| 893 | K.QILGQLDGGSVAV | | 957.514 | 3 | 2870.527 | 2870.526 | 0 | 0.3 | 5294 | 677.3 | 677.3 | 677.3 | 10.98 |
| | LPVVDSTNQYLLDR. | | | | | | | | | | | | |
| | I | | | | | | | | | | | | |
| 964 | K.QILGQLDGGSVAV | | 853.469 | 4 | 3410.853 | 340.853 | 0 | 0.1 | 5294 | 674.3 | 674.3 | 674.3 | 10.79 |
| | LPVVDSTNQYLLDRI | | | | | | | | | | | | |
| | GELK.S | | | | | | | | | | | | |
| 3127 | K.QILGQLDGGSVAV | | 853.715 | 4 | 3411.840 | 3411.837 | 0 | 0.8 | 5294 | 628.0 | 628.0 | 220.5 | 9.79 |
| | LPVVDSTN | | | | | | | | | | | | |
| | [+0.984] | | | | | | | | | | | | |
| | QYLLDRIGELK.SN | | | | | | | | | | | | |
| | [+1] | | | | | | | | | | | | |
| 893 | K.QILGQLDGGSVAV | | 718.387 | 4 | 2870.527 | 2870.526 | 0 | 0.5 | 5294 | 626.7 | 626.7 | 626.7 | 9.98 |
| | LPVVDSTNQYLLDR. | | | | | | | | | | | | |
| | I | | | | | | | | | | | | |
| 893 | K.QILGQLDGGSVAV | | 957.513 | 3 | 2870.523 | 2870.526 | 0 | −0.8 | 5294 | 619.4 | 619.4 | 619.4 | 9.27 |
| | LPVVDSTNQYLLDR. | | | | | | | | | | | | |
| | I | | | | | | | | | | | | |
| 893 | K.QILGQLDGGSVAV | | 957.513 | 3 | 2870.525 | 2870.526 | 0 | 0.2 | 5294 | 591.8 | 591.8 | 591.8 | 8.98 |
| | LPVVDSTNQYLLDR. | | | | | | | | | | | | |
| | I | | | | | | | | | | | | |
| 964 | K.QILGQLDGGSVAV | | 1137.626 | 3 | 3410.864 | 340.853 | 0 | 3.1 | 5294 | 589.2 | 589.2 | 589.2 | 6.94 |
| | LPVVDSTNQYLLDRI | | | | | | | | | | | | |
| | GELK.S | | | | | | | | | | | | |
| 893 | K.QILGQLDGGSVAV | | 957.513 | 3 | 2870.525 | 2870.526 | 0 | 0.2 | 5294 | 580.7 | 580.7 | 580.7 | 8.98 |
| | LPVVDSTNQYLLDR. | | | | | | | | | | | | |
| | I | | | | | | | | | | | | |
| 893 | K.QILGQLDGGSVAV | | 718.388 | 4 | 2870.528 | 2870.526 | 0 | 0.8 | 5294 | 578.3 | 578.3 | 578.3 | 8.98 |
| | LPVVDSTNQYLLDR. | | | | | | | | | | | | |
| | I | | | | | | | | | | | | |
| 1505 | K.QILGQLDGGSVAV | | 957.842 | 3 | 2871.511 | 2871.5 | 0 | 0.4 | 5294 | 571.3 | 571.3 | 61.9 | 8.98 |
| | LPVVDSTN | | | | | | | | | | | | |
| | [+0.984]QYLLD | | | | | | | | | | | | |
| | R.IN[+1] | | | | | | | | | | | | |
| 893 | K.QILGQLDGGSVAV | | 957.514 | 3 | 2870.527 | 2870.526 | 0 | 0.3 | 5294 | 563.7 | 563.7 | 563.7 | 8.98 |
| | LPVVDSTNQYLLDR. | | | | | | | | | | | | |
| | I | | | | | | | | | | | | |
| 893 | K.QILGQLDGGSVAV | | 957.514 | 3 | 2870.528 | 2870.526 | 0 | 0.6 | 5294 | 547.2 | 547.2 | 547.2 | 7.17 |
| | LPVVDSTNQYLLDR. | | | | | | | | | | | | |
| | I | | | | | | | | | | | | |
| 893 | K.QILGQLDGGSVAV | | 957.514 | 3 | 2870.527 | 2870.526 | 0 | 0.3 | 5294 | 545.0 | 545.0 | 545.0 | 8.14 |
| | LPVVDSTNQYLLDR. | | | | | | | | | | | | |
| | I | | | | | | | | | | | | |
| 893 | K.QILGQLDGGSVAV | | 957.514 | 3 | 2870.528 | 2870.526 | 0 | 0.8 | 5294 | 538.9 | 538.9 | 538.9 | 17.17 |
| | LPVVDSTNQYLLDR. | | | | | | | | | | | | |
| | I | | | | | | | | | | | | |
| 893 | K.QILGQLDGGSVAV | | 718.387 | 4 | 2870.528 | 2870.526 | 0 | 0.7 | 5294 | 530.9 | 530.9 | 530.9 | 7.41 |
| | LPVVDSTNQYLLDR. | | | | | | | | | | | | |
| | I | | | | | | | | | | | | |
| 1505 | K.QILGQLDGGSVAV | | 958.516 | 3 | 2873.535 | 2871.5 | 2 | 6.3 | 5294 | 523.0 | 523.0 | 523.0 | 6.06 |
| | LPVVDSTN | | | | | | | | | | | | |
| | [+0.984]QYLL | | | | | | | | | | | | |
| | DR.IN[+1] | | | | | | | | | | | | |
| 893 | K.QILGQLDGGSVAV | | 718.387 | 4 | 2870.528 | 2870.526 | 0 | 0.7 | 5294 | 475.2 | 475.2 | 475.2 | 6.72 |
| | LPVVDSTNQYLLDR. | | | | | | | | | | | | |
| | I | | | | | | | | | | | | |
| 964 | K.QILGQLDGGSVAV | | 1137.624 | 3 | 3410.856 | 340.853 | 0 | 0.9 | 5294 | 472.3 | 472.3 | 472.3 | 6.89 |
| | LPVVDSTNQYLLDRI | | | | | | | | | | | | |
| | GELK.S | | | | | | | | | | | | |
| 964 | K.QILGQLDGGSVAV | | 1137.611 | 3 | 3410.819 | 340.853 | 0 | −9.9 | 5294 | 456.2 | 456.2 | 456.2 | 5.20 |
| | LPVVDSTNQYLLDRI | | | | | | | | | | | | |
| | GELK.S | | | | | | | | | | | | |
| 893 | K.QILGQLDGGSVAV | | 957.514 | 3 | 2870.528 | 2870.526 | 0 | 0.8 | 5294 | 433.3 | 433.3 | 433.3 | 6.22 |
| | LPVVDSTNQYLLDR. | | | | | | | | | | | | |
| | I | | | | | | | | | | | | |
| 893 | K.QILGQLDGGSVAV | | 957.513 | 3 | 2870.525 | 2870.526 | 0 | −0.4 | 5294 | 386.6 | 386.6 | 386.6 | 5.79 |
| | LPVVDSTNQYLLDR. | | | | | | | | | | | | |
| | I | | | | | | | | | | | | |
| 893 | K.QILGQLDGGSVAV | | 957.516 | 3 | 2870.532 | 2870.526 | 0 | 2.2 | 5294 | 385.4 | 385.4 | 385.4 | 6.49 |
| | LPVVDSTNQYLLDR. | | | | | | | | | | | | |
| | I | | | | | | | | | | | | |
| 893 | K.QILGQLDGGSVAV | | 957.513 | 3 | 2870.525 | 2870.526 | 0 | −0.2 | 5294 | 338.9 | 338.9 | 338.9 | 5.35 |
| | LPVVDSTNQYLLDR. | | | | | | | | | | | | |
| | I | | | | | | | | | | | | |
| 1692 | R.IGELKSGDAC | | 1033.503 | 2 | 2065.998 | 2065.997 | 0 | 0.4 | 5321 | 600.8 | 600.8 | 600.8 | 10.77 |
| | [+57.021]IA | | | | | | | | | | | | |
| | EYQQAGR | | | | | | | | | | | | |
| | .GC[+57] | | | | | | | | | | | | |
| 1692 | R.IGELKSGDAC | | 689.337 | 3 | 2065.997 | 2065.997 | 0 | 0.1 | 5321 | 532.8 | 532.8 | 532.8 | 18.93 |
| | [+57.021]IA | | | | | | | | | | | | |
| | EYQQAGR | | | | | | | | | | | | |
| | .GC[+57] | | | | | | | | | | | | |
| 1692 | R.IGELKSGDAC | | 689.337 | 3 | 2065.997 | 2065.997 | 0 | 0.1 | 5321 | 314.4 | 314.4 | 314.4 | 5.58 |
| | [+57.021]IA | | | | | | | | | | | | |
| | EYQQAGR | | | | | | | | | | | | |
| | .GC[+57] | | | | | | | | | | | | |
| 1698 | K.SGDAC | | 763.338 | 2 | 1525.669 | 1525.670 | 0 | 0.6 | 5326 | 851.0 | 851.0 | 851.0 | 15.26 |
| | [+57.021] | | | | | | | | | | | | |
| | IAEYQQAGR.G | | | | | | | | | | | | |
| | C[+57] | | | | | | | | | | | | |
| 1711 | K.SGDAC | | 609.280 | 3 | 1825.826 | 1825.825 | 0 | 0.5 | 5326 | 661.9 | 661.9 | 661.9 | 11.77 |
| | [+57.021] | | | | | | | | | | | | |
| | IAEYQQAGRGSR.G | | | | | | | | | | | | |
| | C[+57] | | | | | | | | | | | | |
| 1711 | K.SGDAC | | 913.416 | 2 | 1825.825 | 1825.825 | 0 | 0.4 | 5326 | 411.6 | 411.6 | 411.6 | 7.40 |
| | [+57.021]I | | | | | | | | | | | | |
| | AEYQQAGRGSR.G | | | | | | | | | | | | |
| | C[+57] | | | | | | | | | | | | |
| 822 | K.RGPAAIGLGPVIG | | 687.738 | 3 | 2061.200 | 2061.200 | 0 | 0.1 | 5364 | 548.2 | 548.2 | 548.2 | 19.12 |
| | IVMAEALR.K | | | | | | | | | | | | |
| 822 | K.RGPAAIGLGPVIG | | 687.738 | 3 | 2061.201 | 2061.200 | 0 | 0.2 | 5364 | 493.4 | 493.4 | 493.4 | 8.91 |
| | IVMAEALR.K | | | | | | | | | | | | |
| 822 | K.RGPAAIGLGPVIG | | 687.739 | 3 | 2061.203 | 2061.200 | 0 | 1.2 | 5364 | 460.7 | 460.7 | 460.7 | 8.67 |
| | IVMAEALR.K | | | | | | | | | | | | |
| 822 | K.RGPAAIGLGPVIG | | 687.739 | 3 | 2061.202 | 2061.200 | 0 | 0.7 | 5364 | 442.4 | 442.4 | 442.4 | 7.94 |
| | IVMAEALR.K | | | | | | | | | | | | |
| 919 | R.VKWPNDLYLQDRK | | 558.970 | 3 | 1674.895 | 1674.896 | 0 | −0.6 | 5393 | 570.2 | 570.2 | 570.2 | 8.38 |
| | .L | | | | | | | | | | | | |
| 912 | R.VKWPNDLYLQDR. | | 516.272 | 3 | 1546.801 | 1546.801 | 0 | 0.2 | 5393 | 559.9 | 467.1 | 467.1 | 7.83 |
| | K | | | | | | | | | | | | |
| 912 | R.VKWPNDLYLQDR. | | 516.272 | 3 | 1546.801 | 1546.801 | 0 | 0.0 | 5393 | 546.6 | 541.2 | 541.2 | 7.83 |
| | K | | | | | | | | | | | | |
| 919 | R.VKWPNDLYLQDRK | | 419.480 | 4 | 1674.896 | 1674.896 | 0 | 0.0 | 5393 | 537.2 | 537.2 | 537.2 | 8.25 |
| | .L | | | | | | | | | | | | |
| 912 | R.VKWPNDLYLQDR. | | 773.905 | 2 | 1546.802 | 1546.801 | 0 | 0.3 | 5393 | 514.6 | 442.0 | 442.0 | 7.62 |
| | K | | | | | | | | | | | | |
| 919 | R.VKWPNDLYLQDRK | | 558.970 | 3 | 1674.896 | 1674.896 | 0 | 0.3 | 5393 | 496.3 | 496.3 | 496.3 | 7.07 |
| | .L | | | | | | | | | | | | |
| 919 | R.VKWPNDLYLQDRK | | 558.970 | 3 | 1674.896 | 1674.896 | 0 | −0.4 | 5393 | 468.7 | 468.7 | 468.7 | 6.12 |
| | .L | | | | | | | | | | | | |
| 912 | R.VKWPNDLYLQDR. | | 516.272 | 3 | 1546.801 | 1546.801 | 0 | 0.0 | 5393 | 468.3 | 468.3 | 468.3 | 7.13 |
| | K | | | | | | | | | | | | |
| 919 | R.VKWPNDLYLQDRK | | 558.970 | 3 | 1674.895 | 1674.896 | 0 | 0.6 | 5393 | 411.7 | 396.4 | 396.4 | 6.01 |
| | .L | | | | | | | | | | | | |
| 4646 | R.VKWPN[+0.984] | N[+1] | 516.935 | 3 | 1548.789 | 1547.785 | 1 | 0.5 | 5393 | 305.1 | 305.1 | 305.1 | 3.33 |
| | DLYLQDR.K | | | | | | | | | | | | |
| 4647 | R.KLAGILVELAGIT | M[+16] | 738.425 | 4 | 2950.679 | 2950.676 | 0 | 1.0 | 5405 | 509.0 | 509.0 | 509.0 | 6.96 |
| | GDAAQIVIGAGINVA | | | | | | | | | | | | |
| | M[+15.995]R.R | | | | | | | | | | | | |
| 4648 | R.KLAGILVELAGIT | | 734.427 | 4 | 2934.685 | 2934.681 | 0 | 1.3 | 5405 | 430.4 | 430.4 | 430.4 | 6.61 |
| | GDAAQIVIGAGINVA | | | | | | | | | | | | |
| | MR.R | | | | | | | | | | | | |
| 4649 | K.LAGILVELAGITG | M[+16] | 941.534 | 3 | 2822.588 | 2822.581 | 0 | 2.6 | 5406 | 603.7 | 603.7 | 603.7 | 7.77 |
| | DAAQIVIGAGINVAM | | | | | | | | | | | | |
| | [+15.995]R.R | | | | | | | | | | | | |
| 1075 | K.LAGILVELAGITG | | 936.200 | 3 | 2806.586 | 2806.586 | 0 | 0.1 | 5406 | 596.1 | 596.1 | 596.1 | 8.98 |
| | DAAQIVIGAGINVAM | | | | | | | | | | | | |
| | R.R | | | | | | | | | | | | |
| 1075 | K.LAGILVELAGITG | | 702.403 | 4 | 2806.588 | 2806.586 | 0 | 0.8 | 5406 | 533.3 | 533.3 | 533.3 | 8.14 |
| | DAAQIVIGAGINVAM | | | | | | | | | | | | |
| | R.R | | | | | | | | | | | | |
| 857 | R.RVEESVVNQGWIT | | 895.737 | 4 | 3579.926 | 3579.924 | 0 | 0.5 | 5435 | 936.9 | 936.9 | 936.9 | 30.00 |
| | LQEAGINLDRNTLAA | | | | | | | | | | | | |
| | TLIR.E | | | | | | | | | | | | |
| 857 | R.RVEESVVNQGWIT | | 716.991 | 5 | 3580.925 | 3579.924 | 1 | 0.6 | 5435 | 873.5 | 755.4 | 132.5 | 14.09 |
| | LQEAGINLDRNTLAA | | | | | | | | | | | | |
| | TLIR.E | | | | | | | | | | | | |
| 857 | R.RVEESVVNQGWIT | | 895.737 | 4 | 3579.926 | 3579.924 | 0 | 0.4 | 5435 | 693.1 | 601.8 | 601.8 | 11.39 |
| | LQEAGINLDRNTLAA | | | | | | | | | | | | |
| | TLIR.E | | | | | | | | | | | | |
| 857 | R.RVEESVVNQGWIT | | 895.736 | 4 | 3579.923 | 3579.924 | 0 | 0.3 | 5435 | 683.4 | 604.3 | 604.3 | 10.68 |
| | LQEAGINLDRNTLAA | | | | | | | | | | | | |
| | TLIR.E | | | | | | | | | | | | |
| 857 | R.RVEESVVNQGWIT | | 895.737 | 4 | 3579.926 | 3579.924 | 0 | 0.5 | 5435 | 660.0 | 576.4 | 576.4 | 11.39 |
| | LQEAGINLDRNTLAA | | | | | | | | | | | | |
| | TLIR.E | | | | | | | | | | | | |
| 857 | R.RVEESVVNQGWIT | | 895.737 | 4 | 3579.926 | 3579.924 | 0 | 0.5 | 5435 | 641.4 | 637.4 | 637.4 | 10.39 |
| | LQEAGINLDRNTLAA | | | | | | | | | | | | |
| | TLIR.E | | | | | | | | | | | | |
| 1067 | R.RVEESVVNQGWIT | | 876.124 | 3 | 2626.356 | 2626.358 | 0 | −0.8 | 5435 | 523.4 | 523.4 | 523.4 | 17.45 |
| | LQEAGINLDR.N | | | | | | | | | | | | |
| 1292 | R.VEESVVNQGWITL | | 856.711 | 4 | 3423.821 | 3423.823 | 0 | 0.5 | 5436 | 839.4 | 839.4 | 839.4 | 13.08 |
| | QEAGINLDRNTLAAT | | | | | | | | | | | | |
| | LIR.E | | | | | | | | | | | | |
| 1292 | R.VEESVVNQGWITL | | 1141.946 | 3 | 3423.824 | 3423.823 | 0 | 0.4 | 5436 | 801.4 | 801.4 | 801.4 | 14.39 |
| | QEAGINLDRNTLAAT | | | | | | | | | | | | |
| | LIR.E | | | | | | | | | | | | |
| 1292 | R.VEESVVNQGWITL | | 856.711 | 4 | 3423.824 | 3423.823 | 0 | 0.3 | 5436 | 795.7 | 795.7 | 795.7 | 12.79 |
| | QEAGINLDRNTLAAT | | | | | | | | | | | | |
| | LIR.E | | | | | | | | | | | | |
| 1292 | R.VEESVVNQGWITL | | 1141.948 | 3 | 3423.828 | 3423.823 | 0 | 1.5 | 5436 | 786.8 | 786.8 | 786.8 | 13.39 |
| | QEAGINLDRNTLAAT | | | | | | | | | | | | |
| | LIR.E | | | | | | | | | | | | |
| 1292 | R.VEESVVNQGWITL | | 685.571 | 5 | 3423.824 | 3423.823 | 0 | 0.2 | 5436 | 673.0 | 673.0 | 673.0 | 10.79 |
| | QEAGINLDRNTLAAT | | | | | | | | | | | | |
| | LIR.E | | | | | | | | | | | | |
| 2188 | R.NTLAATLIR.E | | 486.795 | 2 | 972.583 | 972.584 | 0 | −0.3 | 5458 | 439.6 | 1310.8 | 310.8 | 5.08 |
| 1032 | R.AALELFEQEGLAP | | 787.415 | 3 | 2360.229 | 2360.229 | 0 | 0.4 | 5470 | 684.0 | 684.0 | 684.0 | 11.77 |
| | YLPRWEK.L | | | | | | | | | | | | |
| 1032 | R.AALELFEQEGLAP | | 787.415 | 3 | 2360.230 | 2360.229 | 0 | 0.7 | 5470 | 502.5 | 502.5 | 502.5 | 7.99 |
| | YLPRWEK.L | | | | | | | | | | | | |
| 1914 | R.AALELFEQEGLAP | | 959.011 | 2 | 1917.015 | 1917.012 | 0 | 1.9 | 5470 | 442.1 | 442.1 | 442.1 | 7.94 |
| | YLPR.W | | | | | | | | | | | | |
| 1914 | R.AALELFEQEGLAP | | 639.677 | 3 | 1917.015 | 1917.012 | 0 | 1.7 | 5470 | 375.6 | 375.6 | 375.6 | 7.12 |
| | YLPR.W | | | | | | | | | | | | |
| 1032 | R.AALELFEQEGLAP | | 787.415 | 3 | 2360.231 | 2360.229 | 0 | 0.8 | 5470 | 339.0 | 339.0 | 339.0 | 5.51 |
| | YLPRWEK.L | | | | | | | | | | | | |
| 1914 | R.AALELFEQEGLAP | | 639.675 | 3 | 1917.009 | 1917.012 | 0 | −1.4 | 5470 | 306.0 | 306.0 | 306.0 | 4.47 |
| | YLPR.W | | | | | | | | | | | | |
| 973 | R.WEKLDNFINRPVK | | 553.639 | 3 | 1658.901 | 1658.901 | 0 | 0.1 | 5487 | 520.5 | 520.5 | 520.5 | 7.83 |
| | .L | | | | | | | | | | | | |
| 973 | R.WEKLDNFINRPVK | | 415.481 | 4 | 1658.902 | 1658.901 | 0 | 0.2 | 5487 | 450.3 | 450.3 | 450.3 | 7.38 |
| | .L | | | | | | | | | | | | |
| 973 | R.WEKLDNFINRPVK | | 553.639 | 3 | 1658.901 | 1658.901 | 0 | 0.2 | 5487 | 448.7 | 448.7 | 448.7 | 7.13 |
| | .L | | | | | | | | | | | | |
| 820 | K.LDNFINRPVKLII | | 532.308 | 5 | 2657.513 | 2657.514 | 0 | 0.4 | 5490 | 466.4 | 466.4 | 466.4 | 6.73 |
| | GDKEIFGISR.G | | | | | | | | | | | | |
| 1931 | K.LDNFINRPVK.L | | 405.900 | 3 | 1215.684 | 1215.684 | 0 | 0.0 | 5490 | 433.2 | 433.2 | 433.2 | 6.99 |
| 820 | K.LDNFINRPVKLII | | 532.309 | 5 | 2657.514 | 2657.514 | 0 | −0.1 | 5490 | 300.6 | 300.6 | 300.6 | 5.27 |
| | GDKEIFGISR.G | | | | | | | | | | | | |
| 983 | K.LIIGDKEIFGISR | | 730.927 | 2 | 1460.847 | 1460.847 | 0 | 0.1 | 5500 | 667.7 | 667.7 | 667.7 | 11.40 |
| | .G | | | | | | | | | | | | |
| 983 | K.LIIGDKEIFGISR | | 487.620 | 3 | 1460.847 | 1460.847 | 0 | 0.3 | 5500 | 657.7 | 657.7 | 657.7 | 10.69 |
| | .G | | | | | | | | | | | | |
| 983 | K.LIIGDKEIFGISR | | 487.621 | 3 | 1460.847 | 1460.847 | 0 | 0.0 | 5500 | 604.9 | 604.9 | 604.9 | 10.40 |
| | .G | | | | | | | | | | | | |
| 983 | K.LIIGDKEIFGISR | | 487.621 | 3 | 1460.848 | 1460.847 | 0 | 0.3 | 5500 | 548.8 | 548.8 | 548.8 | 18.56 |
| | .G | | | | | | | | | | | | |
| 983 | K.LIIGDKEIFGISR | | 487.620 | 3 | 1460.846 | 1460.847 | 0 | 0.5 | 5500 | 542.7 | 542.7 | 542.7 | 7.13 |
| | .G | | | | | | | | | | | | |
| 983 | K.LIIGDKEIFGISR | | 487.621 | 3 | 1460.847 | 1460.847 | 0 | −0.2 | 5500 | 436.4 | 436.4 | 436.4 | 7.27 |
| | .G | | | | | | | | | | | | |
| 837 | R.GIDKQGALLLEQD | | 975.196 | 3 | 2923.573 | 2923.571 | 0 | 0.7 | 5513 | 875.9 | 875.9 | 875.9 | 15.35 |
| | GVIKPWMGGEISLR. | | | | | | | | | | | | |
| | S | | | | | | | | | | | | |
| 837 | R.GIDKQGALLLEQD | | 975.195 | 3 | 2923.571 | 2923.571 | 0 | 0.1 | 5513 | 826.4 | 826.4 | 826.4 | 14.39 |
| | GVIKPWMGGEISLR. | | | | | | | | | | | | |
| | S | | | | | | | | | | | | |
| 837 | R.GIDKQGALLLEQD | | 975.195 | 3 | 2923.572 | 2923.571 | 0 | 0.2 | 5513 | 802.2 | 802.2 | 802.2 | 14.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.2 | 5513 | 775.1 | 775.1 | 775.1 | 13.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 759.6 | 759.6 | 759.6 | 13.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.7 | 5513 | 758.9 | 758.9 | 758.9 | 13.39 |
| | GVIKPWMGGEISLR. | | | | | | | | | | | | |
| | S | | | | | | | | | | | | |
| 837 | R.GIDKQGALLLEQD | | 1462.790 | 2 | 2924.573 | 2923.571 | 1 | 0.3 | 5513 | 752.5 | 752.5 | 752.5 | 11.30 |
| | GVIKPWMGGEISLR. | | | | | | | | | | | | |
| | S | | | | | | | | | | | | |
| 837 | R.GIDKQGALLLEQD | | 975.195 | 3 | 2923.570 | 2923.571 | 0 | 0.5 | 5513 | 744.6 | 744.6 | 744.6 | 11.68 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.2 | 5513 | 720.6 | 720.6 | 720.6 | 12.39 |
| | GVIKPWMGGEISLR. | | | | | | | | | | | | |
| | S | | | | | | | | | | | | |
| 837 | R.GIDKQGALLLEQD | | 975.864 | 3 | 2925.576 | 2923.571 | 2 | −0.6 | 5513 | 714.1 | 714.1 | 714.1 | 11.68 |
| | GVIKPWMGGEISLR. | | | | | | | | | | | | |
| | S | | | | | | | | | | | | |
| 837 | R.GIDKQGALLLEQD | | 731.648 | 4 | 2923.569 | 2923.571 | 0 | 0.7 | 5513 | 706.1 | 706.1 | 706.1 | 11.68 |
| | GVIKPWMGGEISLR. | | | | | | | | | | | | |
| | S | | | | | | | | | | | | |
| 1612 | R.GIDKQGALLLEQD | M[+16] | 980.527 | 3 | 2939.566 | 2939.566 | 0 | 0.1 | 5513 | 703.6 | 703.6 | 703.6 | 12.39 |
| | GVIKPWM | | | | | | | | | | | | |
| | [+15.995]GGE | | | | | | | | | | | | |
| | ISLR.S | | | | | | | | | | | | |
| 837 | R.GIDKQGALLLEQD | | 731.648 | 4 | 2923.570 | 2923.571 | 0 | −0.4 | 5513 | 698.4 | 698.4 | 698.4 | 10.68 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.2 | 5513 | 697.4 | 697.4 | 697.4 | 11.39 |
| | GVIKPWMGGEISLR. | | | | | | | | | | | | |
| | S | | | | | | | | | | | | |
| 1612 | R.GIDKQGALLLEQD | M[+16] | 735.647 | 4 | 2939.567 | 2939.566 | 0 | 0.4 | 5513 | 694.0 | 694.0 | 694.0 | 11.39 |
| | GVIKPWM | | | | | | | | | | | | |
| | [+15.995]GG | | | | | | | | | | | | |
| | EISLR.S | | | | | | | | | | | | |
| 837 | R.GIDKQGALLLEQD | | 975.196 | 3 | 2923.572 | 2923.571 | 0 | 0.3 | 5513 | 693.5 | 693.5 | 693.5 | 11.39 |
| | GVIKPWMGGEISLR. | | | | | | | | | | | | |
| | S | | | | | | | | | | | | |
| 837 | R.GIDKQGALLLEQD | | 731.648 | 4 | 2923.571 | 2923.571 | 0 | 0.1 | 5513 | 685.2 | 685.2 | 685.2 | 11.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 679.9 | 679.9 | 679.9 | 11.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.5 | 5513 | 661.7 | 661.7 | 661.7 | 11.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.5 | 5513 | 638.5 | 638.5 | 638.5 | 10.39 |
| | GVIKPWMGGEISLR. | | | | | | | | | | | | |
| | S | | | | | | | | | | | | |
| 837 | R.GIDKQGALLLEQD | | 731.649 | 1 | 2923.573 | 2923.571 | 0 | 0.6 | 5513 | 630.5 | 630.5 | 630.5 | 10.39 |
| | GVIKPWMGGEISLR. | | | | | | | | | | | | |
| | S | | | | | | | | | | | | |
| 837 | R.GIDKQGALLLEQD | | 731.648 | 4 | 2923.571 | 2923.571 | 0 | 0.0 | 5513 | 622.4 | 622.4 | 622.4 | 10.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 617.2 | 617.2 | 617.2 | 10.39 |
| | GVIKPWMGGEISLR. | | | | | | | | | | | | |
| | S | | | | | | | | | | | | |
| 837 | R.GIDKQGALLLEQD | | 731.649 | 4 | 2923.572 | 2923.571 | 0 | 0.3 | 5513 | 611.0 | 611.0 | 611.0 | 10.39 |
| | GVIKPWMGGEISLR. | | | | | | | | | | | | |
| | S | | | | | | | | | | | | |
| 837 | R.GIDKQGALLLEQD | | 731.649 | 4 | 2923.572 | 2923.571 | 0 | 0.4 | 5513 | 600.9 | 600.9 | 600.9 | 10.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.2 | 5513 | 599.0 | 599.0 | 599.0 | 9.39 |
| | GVIKPWMGGEISLR. | | | | | | | | | | | | |
| | S | | | | | | | | | | | | |
| 837 | R.GIDKQGALLLEQD | | 731.648 | 4 | 2923.571 | 2923.571 | 0 | 0.1 | 5513 | 597.7 | 597.7 | 597.7 | 9.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.5 | 5513 | 595.0 | 595.0 | 595.0 | 9.39 |
| | GVIKPWMGGEISLR. | | | | | | | | | | | | |
| | S | | | | | | | | | | | | |
| 837 | R.GIDKQGALLLEQD | | 731.899 | 4 | 2924.575 | 2923.571 | 1 | 0.1 | 5513 | 593.5 | 593.5 | 593.5 | 9.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.5 | 5513 | 591.7 | 1591.7 | 591.7 | 9.39 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.6 | 5513 | 590.5 | 590.5 | 590.5 | 9.39 |
| | GVIKPWMGGEISLR. | | | | | | | | | | | | |
| | S | | | | | | | | | | | | |
| 837 | R.GIDKQGALLLEQD | | 975.196 | 3 | 2923.573 | 2923.571 | 0 | 0.5 | 5513 | 584.2 | 584.2 | 584.2 | 8.66 |
| | GVIKPWMGGEISLR. | | | | | | | | | | | | |
| | S | | | | | | | | | | | | |
| 837 | R.GIDKQGALLLEQD | | 731.899 | 4 | 2924.574 | 2923.571 | 1 | −0.2 | 5513 | 573.4 | 573.4 | 573.4 | 9.39 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 565.6 | 565.6 | 565.6 | 9.39 |
| | GVIKPWMGGEISLR. | | | | | | | | | | | | |
| | S | | | | | | | | | | | | |
| 1612 | R.GIDKQGALLLEQD | M[+16] | 735.648 | 4 | 2939.568 | 2939.566 | 0 | 0.8 | 5513 | 563.2 | 563.2 | 563.2 | 9.39 |
| | GVIKPWM | | | | | | | | | | | | |
| | [+15.995]GG | | | | | | | | | | | | |
| | EISLR.S | | | | | | | | | | | | |
| 837 | R.GIDKQGALLLEQD | | 731.648 | 4 | 2923.572 | 2923.571 | 0 | 0.2 | 5513 | 562.2 | 562.2 | 562.2 | 9.39 |
| | GVIKPWMGGEISLR. | | | | | | | | | | | | |
| | S | | | | | | | | | | | | |
| 1612 | R.GIDKQGALLLEQD | M[+16] | 735.898 | 4 | 2940.570 | 2939.566 | 1 | 0.3 | 5513 | 557.6 | 557.6 | 557.6 | 8.55 |
| | GVIKPWM | | | | | | | | | | | | |
| | [+15.995] | | | | | | | | | | | | |
| | GGEISLR.S | | | | | | | | | | | | |
| 1612 | R.GIDKQGALLLEQD | M[+16] | 735.898 | 4 | 2940.570 | 2939.566 | 1 | 0.1 | 5513 | 557.5 | 557.5 | 557.5 | 7.82 |
| | GVIKPWM | | | | | | | | | | | | |
| | [+15.995]GGE | | | | | | | | | | | | |
| | ISLR.S | | | | | | | | | | | | |
| 837 | R.GIDKQGALLLEQD | | 731.899 | 4 | 2924.576 | 2923.571 | 1 | 0.5 | 5513 | 548.1 | 548.1 | 548.1 | 7.82 |
| | GVIKPWMGGEISLR. | | | | | | | | | | | | |
| | S | | | | | | | | | | | | |
| 837 | R.GIDKQGALLLEQD | | 731.898 | 4 | 2924.571 | 2923.571 | 1 | −1.1 | 5513 | 544.0 | 544.0 | 544.0 | 7.11 |
| | GVIKPWMGGEISLR. | | | | | | | | | | | | |
| | S | | | | | | | | | | | | |
| 837 | R.GIDKQGALLLEQD | | 975.862 | 3 | 2925.571 | 2923.571 | 2 | 2.2 | 5513 | 542.9 | 542.9 | 542.9 | 6.87 |
| | GVIKPWMGGEISLR. | | | | | | | | | | | | |
| | S | | | | | | | | | | | | |
| 837 | R.GIDKQGALLLEQD | | 731.649 | 4 | 2923.572 | 2923.571 | 0 | 0.3 | 5513 | 541.9 | 541.9 | 541.9 | 8.55 |
| | GVIKPWMGGEISLR. | | | | | | | | | | | | |
| | S | | | | | | | | | | | | |
| 837 | R.GIDKQGALLLEQD | | 731.648 | 4 | 2923.571 | 2923.571 | 0 | 0.1 | 5513 | 540.0 | 540.0 | 540.0 | 8.55 |
| | GVIKPWMGGEISLR. | | | | | | | | | | | | |
| | S | | | | | | | | | | | | |
| 837 | R.GIDKQGALLLEQD | | 731.648 | 4 | 2923.571 | 2923.571 | 0 | 0.0 | 5513 | 526.3 | 526.3 | 526.3 | 7.82 |
| | GVIKPWMGGEISLR. | | | | | | | | | | | | |
| | S | | | | | | | | | | | | |
| 837 | R.GIDKQGALLLEQD | | 731.900 | 4 | 2924.578 | 2923.571 | 1 | 1.2 | 5513 | 510.8 | 510.8 | 510.8 | 7.61 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 508.1 | 508.1 | 508.1 | 7.36 |
| | GVIKPWMGGEISLR. | | | | | | | | | | | | |
| | S | | | | | | | | | | | | |
| 1612 | R.GIDKQGALLLEQD | M[+16] | 735.647 | 4 | 2939.567 | 2939.566 | 0 | 0.2 | 5513 | 506.8 | 506.8 | 506.8 | 8.34 |
| | GVIKPWM | | | | | | | | | | | | |
| | [+15.995]GGEI | | | | | | | | | | | | |
| | SLR.S | | | | | | | | | | | | |
| 837 | R.GIDKQGALLLEQD | | 731.649 | 4 | 2923.572 | 2923.571 | 0 | 0.4 | 5513 | 496.9 | 496.9 | 496.9 | 7.36 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.7 | 5513 | 494.8 | 494.8 | 494.8 | 7.61 |
| | GVIKPWMGGEISLR. | | | | | | | | | | | | |
| | S | | | | | | | | | | | | |
| 837 | R.GIDKQGALLLEQD | | 1462.289 | 2 | 2923.570 | 2923.571 | 0 | −0.3 | 5513 | 494.4 | 494.4 | 494.4 | 6.96 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.3 | 5513 | 485.8 | 485.8 | 485.8 | 7.36 |
| | GVIKPWMGGEISLR. | | | | | | | | | | | | |
| | S | | | | | | | | | | | | |
| 837 | R.GIDKQGALLLEQD | | 731.649 | 4 | 2923.574 | 2923.571 | 0 | 0.8 | 5513 | 485.0 | 485.0 | 485.0 | 17.61 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.6 | 5513 | 484.4 | 484.4 | 484.4 | 7.61 |
| | GVIKPWMGGEISLR. | | | | | | | | | | | | |
| | S | | | | | | | | | | | | |
| 837 | R.GIDKQGALLLEQD | | 731.648 | 4 | 2923.572 | 2923.571 | 0 | 0.2 | 5513 | 484.3 | 484.3 | 484.3 | 7.61 |
| | GVIKPWMGGEISLR. | | | | | | | | | | | | |
| | S | | | | | | | | | | | | |
| 837 | R.GIDKQGALLLEQD | | 731.648 | 4 | 2923.569 | 2923.571 | 0 | −0.6 | 5513 | 479.0 | 479.0 | 479.0 | 6.18 |
| | GVIKPWMGGEISLR. | | | | | | | | | | | | |
| | S | | | | | | | | | | | | |
| 837 | R.GIDKQGALLLEQD | | 731.649 | 4 | 2923.572 | 2923.571 | 0 | 0.3 | 5513 | 462.8 | 462.8 | 462.8 | 8.09 |
| | GVIKPWMGGEISLR. | | | | | | | | | | | | |
| | S | | | | | | | | | | | | |
| 1612 | R.GIDKQGALLLEQD | M[+16] | 735.897 | 4 | 2940.567 | 2939.566 | 1 | 0.7 | 5513 | 406.3 | 406.3 | 406.3 | 6.07 |
| | GVIKPWM | | | | | | | | | | | | |
| | [+15.995]G | | | | | | | | | | | | |
| | GEISLR.S | | | | | | | | | | | | |
| 837 | R.GIDKQGALLLEQD | | 975.195 | 3 | 2923.570 | 2923.571 | 0 | 0.3 | 5513 | 397.9 | 397.9 | 397.9 | 6.43 |
| | GVIKPWMGGEISLR. | | | | | | | | | | | | |
| | S | | | | | | | | | | | | |
| 837 | R.GIDKQGALLLEQD | | 731.899 | 4 | 2924.575 | 2923.571 | 1 | 0.3 | 5513 | 395.2 | 395.2 | 395.2 | 6.67 |
| | GVIKPWMGGEISLR. | | | | | | | | | | | | |
| | S | | | | | | | | | | | | |
| 837 | R.GIDKQGALLLEQD | | 731.650 | 4 | 2923.578 | 2923.571 | 0 | 2.3 | 5513 | 374.0 | 374.0 | 374.0 | 6.90 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.6 | 5513 | 355.8 | 355.8 | 355.8 | 5.51 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.6 | 5513 | 332.0 | 332.0 | 332.0 | 5.28 |
| | GVIKPWMGGEISLR. | | | | | | | | | | | | |
| | S | | | | | | | | | | | | |
| 837 | R.GIDKQGALLLEQD | | 731.649 | 4 | 2923.573 | 2923.571 | 0 | 0.5 | 5513 | 326.9 | 326.9 | 326.9 | 5.51 |
| | GVIKPWMGGEISLR. | | | | | | | | | | | | |
| | S | | | | | | | | | | | | |
| 837 | R.GIDKQGALLLEQD | | 732.150 | 4 | 2925.579 | 2923.571 | 2 | 0.3 | 5513 | 307.5 | 307.5 | 307.5 | 5.04 |
| | GVIKPWMGGEISLR. | | | | | | | | | | | | |
| | S | | | | | | | | | | | | |
| 915 | K.QGALLLEQDGVIK | | 837.452 | 3 | 2510.342 | 2510.344 | 0 | −0.6 | 5517 | 325.8 | 194.5 | 194.5 | 4.35 |
| | PWMGGEISLR.S | | | | | | | | | | | | |
| 864 | R.SAEKLQLPPLER. | | 690.896 | 2 | 1380.784 | 1380.785 | 0 | 0.3 | 5540 | 695.3 | 695.3 | 695.3 | 10.69 |
| | L | | | | | | | | | | | | |
| 864 | R.SAEKLQLPPLER. | | 690.896 | 2 | 1380.785 | 1380.785 | 0 | 0.1 | 5540 | 646.2 | 646.2 | 646.2 | 10.40 |
| | L | | | | | | | | | | | | |
| 864 | R.SAEKLQLPPLER. | | 690.896 | 2 | 1380.784 | 1380.785 | 0 | −0.3 | 5540 | 643.0 | 643.0 | 643.0 | 9.69 |
| | L | | | | | | | | | | | | |
| 864 | R.SAEKLQLPPLER. | | 690.896 | 2 | 1380.784 | 1380.785 | 0 | 0.3 | 5540 | 623.6 | 623.6 | 623.6 | 9.69 |
| | L | | | | | | | | | | | | |
| 864 | R.SAEKLQLPPLER. | | 690.896 | 2 | 1380.785 | 1380.785 | 0 | 0.1 | 5540 | 603.3 | 603.3 | 603.3 | 10.40 |
| | L | | | | | | | | | | | | |
| 864 | R.SAEKLQLPPLER. | | 460.933 | 3 | 1380.784 | 1380.785 | 0 | −0.1 | 5540 | 543.5 | 543.5 | 543.5 | 7.83 |
| | L | | | | | | | | | | | | |
| 864 | R.SAEKLQLPPLER. | | 460.933 | 3 | 1380.785 | 1380.785 | 0 | 0.0 | 5540 | 492.2 | 492.2 | 492.2 | 7.62 |
| | L | | | | | | | | | | | | |
| 864 | R.SAEKLQLPPLER. | | 460.933 | 3 | 1380.785 | 1380.785 | 0 | 0.1 | 5540 | 462.2 | 462.2 | 462.2 | 7.13 |
| | L | | | | | | | | | | | | |
| 864 | R.SAEKLQLPPLER. | | 460.933 | 3 | 1380.785 | 1380.785 | 0 | 0.4 | 5540 | 404.0 | 404.0 | 404.0 | 7.03 |
| | L | | | | | | | | | | | | |
| 864 | R.SAEKLQLPPLER. | | 460.933 | 3 | 1380.785 | 1380.785 | 0 | 0.1 | 5540 | 401.2 | 401.2 | 401.2 | 7.03 |
| | L | | | | | | | | | | | | |
| 4650 | R.SAEKLQLPPLERL | | 912.017 | 2 | 1823.026 | 1823.027 | 0 | −0.5 | 5540 | 392.7 | 392.7 | 392.7 | 6.26 |
| | TLD.- | | | | | | | | | | | | |
| 864 | R.SAEKLQLPPLER. | | 460.933 | 3 | 1380.785 | 1380.785 | 0 | 0.1 | 5540 | 371.0 | 371.0 | 371.0 | 6.91 |
| | L | | | | | | | | | | | | |
| 864 | R.SAEKLQLPPLER. | | 460.933 | 3 | 1380.785 | 1380.785 | 0 | 0.0 | 5540 | 356.1 | 356.1 | 356.1 | 5.52 |
| | L | | | | | | | | | | | | |
| 4651 | K.LQLPPLERLTLD. | | 704.414 | 2 | 1407.821 | 1407.821 | 0 | 0.0 | 5544 | 459.2 | 459.2 | 459.2 | 7.38 |
| | - | | | | | | | | | | | | |
| 1905 | K.LQLPPLER.L | | 483.293 | 2 | 965.578 | 965.578 | 0 | 0.0 | 5544 | 402.1 | 402.1 | 402.1 | 6.99 |
| 1905 | K.LQLPPLER.L | | 483.293 | 2 | 965.578 | 965.578 | 0 | 0.0 | 5544 | 378.4 | 378.4 | 378.4 | 6.72 |
| 1905 | K.LQLPPLER.L | | 483.293 | 2 | 965.578 | 965.578 | 0 | 0.2 | 5544 | 351.1 | 351.1 | 351.1 | 5.33 |

TABLE 8
RNF213 Peptides Rep 2

| | | | | | | | | | Start- | | | | |
|---|---|---|---|---|---|---|---|---|---|---|---|---|---|
| SEQ | | Modifi- | | | | Calc. | Off- | Mass | ing | | | | |
| ID | Peptide | cation | Observed | | Observed | mass | by-x | error | posi- | | | Delta | \|Log |
| NO: | <ProteinMetrics Confidential> | Type(s) | m/z | Z | (M + H) | (M + H) | error | (ppm) | tion | Score | Delta | Mod | Prob\| |
| 880 | K.ASWTVQESK.K | | 518.259 | 2 | 1035.511 | 1035.511 | 0 | 0.1 | 81 | 454.7 | 454.7 | 454.7 | 7.76 |
| 880 | K.ASWTVQESK.K | | 518.259 | 2 | 1035.510 | 1035.511 | 0 | 0.4 | 81 | 392.3 | 392.3 | 392.3 | 6.64 |
| 884 | R.GLSQEGTGPPTSAGEGHSR.T | | 608.955 | 3 | 1824.849 | 1824.847 | 0 | 1.1 | 215 | 782.9 | 782.9 | 782.9 | 13.98 |
| 884 | R.GLSQEGTGPPTSAGEGHSR.T | | 912.928 | 2 | 1824.848 | 1824.847 | 0 | 0.3 | 215 | 774.1 | 774.1 | 774.1 | 13.98 |
| 884 | R.GLSQEGTGPPTSAGEGHSR.T | | 608.954 | 3 | 1824.847 | 1824.847 | 0 | 0.2 | 215 | 675.0 | 675.0 | 675.0 | 11.98 |
| 1079 | R.GLSQEGTGPPTSAGEGHSRTEDAAQELLLPESK.G | | 1117.211 | 3 | 3349.617 | 3349.614 | 0 | 0.9 | 215 | 637.5 | 637.5 | 637.5 | 9.99 |
| 884 | R.GLSQEGTGPPTSAGEGHSR.T | | 608.954 | 3 | 1824.847 | 1824.847 | 0 | 0.1 | 215 | 565.5 | 565.5 | 565.5 | 9.38 |
| 884 | R.GLSQEGTGPPTSAGEGHSR.T | | 608.954 | 3 | 1824.847 | 1824.847 | 0 | 0.1 | 215 | 501.2 | 501.2 | 501.2 | 9.07 |
| 1079 | R.GLSQEGTGPPTSAGEGHSRTEDAAQELLLPESK.G | | 1117.210 | 3 | 3349.614 | 3349.614 | 0 | 0.1 | 215 | 599.6 | 599.6 | 599.6 | 8.99 |
| 1079 | R.GLSQEGTGPPTSAGEGHSRTEDAAQELLLPESK.G | | 1117.210 | 3 | 3349.616 | 3349.614 | 0 | 0.6 | 215 | 578.5 | 578.5 | 578.5 | 8.99 |
| 884 | R.GLSQEGTGPPTSAGEGHSR.T | | 608.954 | 3 | 1824.848 | 1824.847 | 0 | 0.6 | 215 | 383.6 | 383.6 | 383.6 | 7.97 |
| 1079 | R.GLSQEGTGPPTSAGEGHSRTEDAAQELLLPESK.G | | 838.159 | 4 | 3349.614 | 3349.614 | 0 | 0.2 | 215 | 506.9 | 506.9 | 506.9 | 7.50 |
| 1079 | R.GLSQEGTGPPTSAGEGHSRTEDAAQELLLPESK.G | | 838.159 | 4 | 3349.614 | 3349.614 | 0 | 0.2 | 215 | 496.8 | 496.8 | 496.8 | 7.50 |
| 884 | R.GLSQEGTGPPTSAGEGHSR.T | | 456.967 | 4 | 1824.846 | 1824.847 | 0 | 0.5 | 215 | 449.4 | 449.4 | 449.4 | 7.21 |
| 842 | R.GLSQEGTGPPTSAGEGHSRTEDAAQELLLPESKGGSSEPGTELQT | | 970.894 | 7 | 6790.213 | 6790.214 | 0 | −0.3 | 215 | 603.1 | 603.1 | 603.1 | 6.34 |
| | TEQQAGASASMAVDAVAEPANAVK.G | | | | | | | | | | | | |
| 1079 | R.GLSQEGTGPPTSAGEGHSRTEDAAQELLLPESK.G | | 670.729 | 5 | 3349.617 | 3349.614 | 0 | 0.7 | 215 | 475.6 | 475.6 | 475.6 | 6.33 |
| 1079 | R.GLSQEGTGPPTSAGEGHSRTEDAAQELLLPESK.G | | 838.159 | 4 | 3349.614 | 3349.614 | 0 | −0.2 | 215 | 410.1 | 410.1 | 410.1 | 6.04 |
| 1079 | R.GLSQEGTGPPTSAGEGHSRTEDAAQELLLPESK.G | | 670.729 | 5 | 3349.616 | 3349.614 | 0 | 0.5 | 215 | 383.0 | 383.0 | 383.0 | 5.99 |
| 1079 | R.GLSQEGTGPPTSAGEGHSRTEDAAQELLLPESK.G | | 670.730 | 5 | 3349.618 | 3349.614 | 0 | 1.2 | 215 | 376.8 | 376.8 | 376.8 | 5.99 |
| 1467 | R.GLSQEGTGPPTSAGEGHS[+79.966]RTEDAAQELLLPESK.G | S[+80] | 858.150 | 4 | 3429.578 | 3429.581 | 0 | −0.8 | 215 | 399.1 | 399.1 | 48.1 | 5.92 |
| 842 | R.GLSQEGTGPPTSAGEGHSRTEDAAQELLLPESKGGSSEPGTELQT | | 1358.849 | 5 | 6790.216 | 6790.214 | 0 | 0.2 | 215 | 466.2 | 466.2 | 466.2 | 5.38 |
| | TEQQAGASASMAVDAVAEPANAVK.G | | | | | | | | | | | | |
| 1185 | R.TEDAAQELLLPESK.G | | 772.396 | 2 | 1543.785 | 1543.785 | 0 | 0.3 | 234 | 642.2 | 559.6 | 559.6 | 10.72 |
| 2915 | R.TEDAAQELLLPESKGGSSEPGTELQTTEQQAGASASMAVDAVAEP | | 1246.851 | 4 | 4984.384 | 4984.385 | 0 | −0.3 | 234 | 669.5 | 669.5 | 669.5 | 8.55 |
| | ANAVK.G | | | | | | | | | | | | |
| 1185 | R.TEDAAQELLLPESK.G | | 772.396 | 2 | 1543.785 | 1543.785 | 0 | −0.3 | 234 | 538.3 | 488.8 | 488.8 | 8.40 |
| 1185 | R.TEDAAQELLLPESK.G | | 515.267 | 3 | 1543.785 | 1543.785 | 0 | 0.2 | 234 | 411.2 | 377.8 | 377.8 | 7.02 |
| 1474 | R.TEDAAQELLLPESKGGS[+79.966]SEPGTELQT[+79.966]T | N[+1], | 1045.867 | 5 | 5225.303 | 5225.268 | 0 | 6.7 | 234 | 410.6 | 410.6 | 6.7 | 3.58 |
| | [+79.966]EQQAGASASMAVDAVAEPAN[+0.984]AVK.G | S[+80], | | | | | | | | | | | |
| | | T[+80]* | | | | | | | | | | | |
| | | 2 | | | | | | | | | | | |
| 1210 | K.GGSSEPGTELQTTEQQAGASASMAVDAVAEPANAVKGAGKEMK.E | | 1387.660 | 3 | 4160.964 | 4160.971 | 0 | −1.7 | 248 | 1006.2 | 949.1 | 949.1 | 30.00 |
| 1078 | K.GGSSEPGTELQTTEQQAGASASMAVDAVAEPANAVKGAGK.E | | 1258.268 | 3 | 3772.790 | 3772.793 | 0 | −0.8 | 248 | 920.2 | 920.2 | 920.2 | 15.35 |
| 1210 | K.GGSSEPGTELQTTEQQAGASASMAVDAVAEPANAVKGAGKEMK.E | | 1040.999 | 4 | 4160.974 | 4160.971 | 0 | 0.6 | 248 | 856.6 | 856.6 | 856.6 | 14.20 |
| 1210 | K.GGSSEPGTELQTTEQQAGASASMAVDAVAEPANAVKGAGKEMK.E | | 1387.995 | 3 | 4161.970 | 4160.971 | 1 | −1.1 | 248 | 867.9 | 867.9 | 681.3 | 13.61 |
| 1078 | K.GGSSEPGTELQTTEQQAGASASMAVDAVAEPANAVKGAGK.E | | 1258.269 | 3 | 3772.792 | 3772.793 | 0 | −0.4 | 248 | 811.4 | 811.4 | 811.4 | 13.41 |
| 1210 | K.GGSSEPGTELQTTEQQAGASASMAVDAVAEPANAVKGAGKEMK.E | | 1387.664 | 3 | 4160.978 | 4160.971 | 0 | 1.7 | 248 | 819.5 | 819.5 | 819.5 | 13.19 |
| 1066 | K.GGSSEPGTELQTTEQQAGASASMAVDAVAEPANAVK.G | | 1153.878 | 3 | 3459.619 | 3459.618 | 0 | 0.2 | 248 | 751.2 | 751.2 | 751.2 | 12.75 |
| 1078 | K.GGSSEPGTELQTTEQQAGASASMAVDAVAEPANAVKGAGK.E | | 755.365 | 5 | 3772.794 | 3772.793 | 0 | 0.1 | 248 | 783.9 | 783.9 | 783.9 | 12.47 |
| 1078 | K.GGSSEPGTELQTTEQQAGASASMAVDAVAEPANAVKGAGK.E | | 943.952 | 4 | 3772.785 | 3772.793 | 0 | −2.1 | 248 | 777.8 | 777.8 | 777.8 | 11.89 |
| 1066 | K.GGSSEPGTELQTTEQQAGASASMAVDAVAEPANAVK.G | | 1153.878 | 3 | 3459.619 | 3459.618 | 0 | 0.2 | 248 | 743.5 | 743.5 | 743.5 | 11.75 |
| 1567 | K.GGSSEPGTELQTTEQQAGASASM[+15.995]AVDAVAEPANAVK. | M[+16] | 1159.210 | 3 | 3475.616 | 3475.613 | 0 | 0.7 | 248 | 721.8 | 721.8 | 721.8 | 11.75 |
| | G | | | | | | | | | | | | |
| 1066 | K.GGSSEPGTELQTTEQQAGASASMAVDAVAEPANAVK.G | | 1153.878 | 3 | 3459.620 | 3459.618 | 0 | 0.5 | 248 | 701.1 | 694.2 | 694.2 | 11.75 |
| 1078 | K.GGSSEPGTELQTTEQQAGASASMAVDAVAEPANAVKGAGK.E | | 943.954 | 4 | 3772.795 | 3772.793 | 0 | 0.5 | 248 | 740.2 | 740.2 | 740.2 | 11.47 |
| 1210 | K.GGSSEPGTELQTTEQQAGASASMAVDAVAEPANAVKGAGKEMK.E | | 1041.250 | 4 | 4161.978 | 4160.971 | 1 | 0.8 | 248 | 735.9 | 735.9 | 484.3 | 11.20 |
| 1210 | K.GGSSEPGTELQTTEQQAGASASMAVDAVAEPANAVKGAGKEMK.E | | 1040.999 | 4 | 4160.973 | 4160.971 | 0 | 0.3 | 248 | 718.4 | 564.8 | 564.8 | 11.20 |
| 1066 | K.GGSSEPGTELQTTEQQAGASASMAVDAVAEPANAVK.G | | 865.660 | 4 | 3459.618 | 3459.618 | 0 | 0.1 | 248 | 678.0 | 678.0 | 678.0 | 10.75 |
| 1567 | K.GGSSEPGTELQTTEQQAGASASM[+15.995]AVDAVAEPANAVK. | M[+16] | 1159.209 | 3 | 3475.614 | 3475.613 | 0 | 0.2 | 248 | 659.7 | 659.7 | 659.7 | 10.75 |
| | G | | | | | | | | | | | | |
| 1078 | K.GGSSEPGTELQTTEQQAGASASMAVDAVAEPANAVKGAGK.E | | 943.955 | 4 | 3772.797 | 3772.793 | 0 | 1.1 | 248 | 696.4 | 696.4 | 696.4 | 10.47 |
| 1567 | K.GGSSEPGTELQTTEQQAGASASM[+15.995]AVDAVAEPANAVK. | M[+16] | 1159.208 | 3 | 3475.610 | 3475.613 | 0 | −0.7 | 248 | 699.6 | 694.3 | 694.3 | 10.16 |
| | G | | | | | | | | | | | | |
| 1066 | K.GGSSEPGTELQTTEQQAGASASMAVDAVAEPANAVK.G | | 1154.546 | 3 | 3461.623 | 3459.618 | 2 | −0.6 | 248 | 672.1 | 638.8 | 375.1 | 10.16 |
| 1066 | K.GGSSEPGTELQTTEQQAGASASMAVDAVAEPANAVK.G | | 865.661 | 4 | 3459.620 | 3459.618 | 0 | 0.7 | 248 | 656.7 | 656.7 | 656.7 | 10.15 |
| 1210 | K.GGSSEPGTELQTTEQQAGASASMAVDAVAEPANAVKGAGKEMK.E | | 694.335 | 6 | 4160.973 | 4160.971 | 0 | 0.3 | 248 | 696.8 | 696.8 | 696.8 | 9.68 |
| 1567 | K.GGSSEPGTELQTTEQQAGASASM[+15.995]AVDAVAEPANAVK. | M[+16] | 1159.878 | 3 | 3477.620 | 3475.613 | 2 | 0.1 | 248 | 591.8 | 591.8 | 288.2 | 8.75 |
| | G | | | | | | | | | | | | |
| 1066 | K.GGSSEPGTELQTTEQQAGASASMAVDAVAEPANAVK.G | | 1154.211 | 3 | 3460.620 | 3459.618 | 1 | −0.5 | 248 | 566.2 | 533.2 | 369.0 | 8.16 |
| 1066 | K.GGSSEPGTELQTTEQQAGASASMAVDAVAEPANAVK.G | | 1154.212 | 3 | 3460.622 | 3459.618 | 1 | 0.3 | 248 | 567.0 | 567.0 | 392.4 | 8.15 |
| 1210 | K.GGSSEPGTELQTTEQQAGASASMAVDAVAEPANAVKGAGKEMK.E | | 694.335 | 6 | 4160.971 | 4160.971 | 0 | 0.1 | 248 | 637.9 | 487.8 | 487.8 | 8.08 |
| 1066 | K.GGSSEPGTELQTTEQQAGASASMAVDAVAEPANAVK.G | | 866.161 | 4 | 3461.621 | 3459.618 | 2 | −1.1 | 248 | 597.6 | 425.5 | 274.4 | 7.57 |
| 1066 | K.GGSSEPGTELQTTEQQAGASASMAVDAVAEPANAVK.G | | 1154.211 | 3 | 3460.620 | 3459.618 | 1 | −0.5 | 248 | 582.9 | 567.6 | 380.3 | 7.57 |
| 1066 | K.GGSSEPGTELQTTEQQAGASASMAVDAVAEPANAVK.G | | 1154.211 | 3 | 3460.620 | 3459.618 | 1 | 0.5 | 248 | 586.4 | 486.7 | 345.6 | 7.28 |
| 1210 | K.GGSSEPGTELQTTEQQAGASASMAVDAVAEPANAVKGAGKEMK.E | | 832.999 | 5 | 4160.966 | 4160.971 | 0 | −1.3 | 248 | 613.2 | 396.9 | 396.9 | 7.21 |
| 1210 | K.GGSSEPGTELQTTEQQAGASASMAVDAVAEPANAVKGAGKEMK.E | | 833.201 | 5 | 4161.975 | 4160.971 | 1 | 0.2 | 248 | 560.7 | 454.8 | 293.9 | 6.79 |
| 1567 | K.GGSSEPGTELQTTEQQAGASASM[+15.995]AVDAVAEPANAVK. | M[+16] | 869.659 | 4 | 3475.613 | 3475.613 | 0 | −0.1 | 248 | 484.2 | 484.2 | 484.2 | 6.76 |
| | G | | | | | | | | | | | | |
| 1066 | K.GGSSEPGTELQTTEQQAGASASMAVDAVAEPANAVK.G | | 1154.212 | 3 | 3460.623 | 3459.618 | 1 | 0.4 | 248 | 476.0 | 363.5 | 282.0 | 6.60 |
| 1066 | K.GGSSEPGTELQTTEQQAGASASMAVDAVAEPANAVK.G | | 1154.213 | 3 | 3460.624 | 3459.618 | 1 | 0.8 | 248 | 449.6 | 449.6 | 278.8 | 6.60 |
| 1066 | K.GGSSEPGTELQTTEQQAGASASMAVDAVAEPANAVK.G | | 1153.877 | 3 | 3459.618 | 3459.618 | 0 | −0.1 | 248 | 403.9 | 403.9 | 403.9 | 6.18 |
| 1066 | K.GGSSEPGTELQTTEQQAGASASMAVDAVAEPANAVK.G | | 1154.214 | 3 | 3460.626 | 3459.618 | 1 | 1.4 | 248 | 421.6 | 374.2 | 99.2 | 5.89 |
| 1066 | K.GGSSEPGTELQTTEQQAGASASMAVDAVAEPANAVK.G | | 1153.882 | 3 | 3459.632 | 3459.618 | 0 | 4.1 | 248 | 471.9 | 447.1 | 447.1 | 5.00 |
| 1066 | K.GGSSEPGTELQTTEQQAGASASMAVDAVAEPANAVK.G | | 692.729 | 5 | 3459.617 | 3459.618 | 0 | 0.3 | 248 | 447.5 | 447.5 | 447.5 | 4.76 |
| 1066 | K.GGSSEPGTELQTTEQQAGASASMAVDAVAEPANAVK.G | | 1153.880 | 3 | 3459.626 | 3459.618 | 0 | 2.3 | 248 | 403.0 | 325.8 | 325.8 | 4.62 |
| 1066 | K.GGSSEPGTELQTTEQQAGASASMAVDAVAEPANAVK.G | | 1154.214 | 3 | 3460.628 | 3459.618 | 1 | 2.0 | 248 | 328.5 | 287.5 | 237.9 | 4.24 |
| 1066 | K.GGSSEPGTELQTTEQQAGASASMAVDAVAEPANAVK.G | | 1153.876 | 3 | 3459.614 | 3459.618 | C | −1.3 | 248 | 388.7 | 301.9 | 301.9 | 3.75 |
| 1066 | K.GGSSEPGTELQTTEQQAGASASMAVDAVAEPANAVK.G | | 1153.877 | 3 | 3459.617 | 3459.618 | 0 | −0.4 | 248 | 349.4 | 248.0 | 248.0 | 3.57 |
| 1567 | K.GGSSEPGTELQTTEQQAGASASM[+15.995]AVDAVAEPANAVK. | M[+16] | 869.658 | 4 | 3475.610 | 3475.613 | 0 | −0.9 | 248 | 321.2 | 197.1 | 197.1 | 3.31 |
| | G | | | | | | | | | | | | |
| 1066 | K.GGSSEPGTELQTTEQQAGASASMAVDAVAEPANAVK.G | | 1154.550 | 3 | 3461.636 | 3459.618 | 2 | 3.4 | 248 | 310.8 | 242.9 | 242.9 | 2.63 |
| 1725 | R.MKQPPATTPPFKTHC[+57.021]QEAETK.T | C[+57] | 607.551 | 4 | 2427.182 | 2427.180 | 0 | 1.1 | 296 | 627.1 | 627.1 | 627.1 | 10.36 |
| 1725 | R.MKQPPATTPPFKTHC[+57.021]QEAETK.T | C[+57] | 607.550 | 4 | 2427.180 | 2427.180 | 0 | 0.2 | 296 | 603.2 | 603.2 | 603.2 | 10.36 |
| 1662 | R.MKQPPATT[+79.966]PPFKTHC[+57.021]QEAETK.T | C[+57], | 627.793 | 4 | 2508.149 | 2507.146 | 1 | −0.1 | 296 | 581.3 | 581.3 | 48.3 | 9.36 |
| | | T[+80] | | | | | | | | | | | |
| 1725 | R.MKQPPATTPPFKTHC[+57.021]QEAETK.T | C[+57] | 486.242 | 5 | 2427.183 | 2427.180 | 0 | 1.3 | 296 | 568.1 | 568.1 | 568.1 | 9.36 |
| 1725 | R.MKQPPATTPPFKTHC[+57.021]QEAETK.T | C[+57] | 809.732 | 3 | 2427.181 | 2427.180 | 0 | 0.7 | 296 | 618.5 | 618.5 | 618.5 | 8.94 |
| 1666 | R.M[+15.995]KQPPATTPPFKTHC[+57.021]QEAETK.T | C[+57], | 611.550 | 4 | 2443.176 | 2443.174 | 0 | 0.6 | 296 | 543.2 | 543.2 | 543.2 | 8.62 |
| | | M[+16] | | | | | | | | | | | |
| 1666 | R.M[+15.995]KQPPATTPPFKTHC[+57.021]QEAETK.T | C[+57], | 611.549 | 4 | 2443.175 | 2443.174 | 0 | 0.1 | 296 | 484.4 | 484.4 | 484.4 | 8.44 |
| | | M[+16] | | | | | | | | | | | |
| 1666 | R.M[+15.995]KQPPATTPPFKTHC[+57.021]QEAETK.T | C[+57], | 489.441 | 5 | 2443.176 | 2443.174 | 0 | 0.6 | 296 | 474.7 | 474.7 | 474.7 | 8.29 |
| | | M[+16] | | | | | | | | | | | |
| 824 | R.MKQPPATTPPFK.T | | 671.863 | 2 | 1342.719 | 1342.719 | 0 | 0.1 | 296 | 539.3 | 539.3 | 539.3 | 8.12 |
| 824 | R.MKQPPATTPPFK.T | | 448.244 | 3 | 1342.719 | 1342.719 | 0 | 0.0 | 296 | 453.6 | 453.6 | 453.6 | 7.49 |
| 1666 | R.M[+15.995]KQPPATTPPFKTHC[+57.021]QEAETK.T | C[+57], | 489.441 | 5 | 2443.177 | 2443.174 | 0 | 0.9 | 296 | 422.8 | 422.8 | 422.8 | 7.47 |
| | | M[+16] | | | | | | | | | | | |
| 824 | R.MKQPPATTPPFK.T | | 671.863 | 2 | 1342.718 | 1342.719 | 0 | −0.7 | 296 | 515.1 | 515.1 | 515.1 | 7.35 |
| 824 | R.MKQPPATTPPFK.T | | 671.863 | 2 | 1342.718 | 1342.719 | 0 | 0.7 | 296 | 489.2 | 489.2 | 489.2 | 7.35 |
| 1539 | R.M[+15.995]KQPPATTPPFK.T | M[+16] | 679.861 | 2 | 1358.715 | 1358.714 | 0 | 0.6 | 296 | 420.5 | 420.5 | 420.5 | 7.26 |
| 1666 | R.M[+15.995]KQPPATTPPFKTHC[+57.021]QEAETK.T | C[+57], | 611.551 | 4 | 2443.181 | 2443.174 | 0 | 2.6 | 296 | 464.1 | 464.1 | 464.1 | 6.88 |
| | | M[+16] | | | | | | | | | | | |
| 824 | R.MKQPPATTPPFK.T | | 448.244 | 3 | 1342.718 | 1342.719 | 0 | −0.8 | 296 | 417.4 | 417.4 | 417.4 | 6.49 |
| 1539 | R.M[+15.995]KQPPATTPPFK.T | M[+16] | 453.576 | 3 | 1358.714 | 1358.714 | 0 | 0.3 | 296 | 405.8 | 318.9 | 318.9 | 5.83 |
| 1539 | R.M[+15.995]KQPPATTPPFK.T | M[+16] | 453.576 | 3 | 1358.714 | 1358.714 | 0 | 0.0 | 296 | 417.9 | 261.2 | 261.2 | 5.73 |
| 1666 | R.M[+15.995]KQPPATTPPFKTHC[+57.021]QEAETK.T | C[+57], | 815.062 | 3 | 2443.172 | 2443.174 | 0 | −1.0 | 296 | 432.2 | 432.2 | 432.2 | 5.46 |
| | | M[+16] | | | | | | | | | | | |
| 1539 | R.M[+15.995]KQPPATTPPFK.T | M[+16] | 679.861 | 2 | 1358.714 | 1358.714 | 0 | 0.4 | 296 | 321.6 | 229.0 | 229.0 | 5.38 |
| 1666 | R.M[+15.995]KQPPATTPPFKTHC[+57.021]QEAETK.T | C[+57], | 489.440 | 5 | 2443.172 | 2443.174 | 0 | −0.9 | 296 | 321.3 | 321.3 | 321.3 | 4.96 |
| | | M[+16] | | | | | | | | | | | |
| 1539 | R.M[+15.995]KQPPATTPPFK.T | M[+16] | 453.576 | 3 | 1358.713 | 1358.714 | 0 | −0.3 | 296 | 322.6 | 220.1 | 220.1 | 4.79 |
| 1539 | R.M[+15.995]KQPPATTPPFK.T | M[+16] | 453.576 | 3 | 1358.713 | 1358.714 | 0 | −0.2 | 296 | 361.2 | 147.0 | 147.0 | 4.74 |
| 1539 | R.M[+15.995]KQPPATTPPFK.T | M[+16] | 453.576 | 3 | 1358.713 | 1358.714 | 0 | −0.2 | 296 | 314.1 | 204.1 | 204.1 | 4.72 |
| 1690 | K.THC[+57.021]QEAETKTKDEMAAAEEK.V | C[+57] | 769.680 | 3 | 2307.024 | 2307.023 | 0 | 0.6 | 308 | 794.5 | 794.5 | 794.5 | 11.94 |
| 1690 | K.THC[+57.021]QEAETKTKDEMAAAEEK.V | C[+57] | 577.511 | 4 | 2307.023 | 2307.023 | 0 | 0.0 | 308 | 689.2 | 689.2 | 689.2 | 11.36 |
| 1667 | K.THC[+57.021]QEAETKTKDEM[+15.995]AAAEEK.V | C[+57], | 581.510 | 4 | 2323.018 | 2323.018 | 0 | 0.0 | 308 | 512.4 | 512.4 | 512.4 | 7.85 |
| | | M[+16] | | | | | | | | | | | |
| 1667 | K.THC[+57.021]QEAETKTKDEM[+15.995]AAAEEK.V | C[+57], | 581.510 | 4 | 2323.019 | 2323.018 | 0 | 0.4 | 308 | 373.5 | 373.5 | 373.5 | 7.06 |
| | | M[+16] | | | | | | | | | | | |
| 1690 | K.THC[+57.021]QEAETKTKDEMAAAEEK.V | C[+57] | 462.209 | 5 | 2307.018 | 2307.023 | 0 | −2.1 | 308 | 349.9 | 349.9 | 349.9 | 4.96 |
| 1690 | K.THC[+57.021]QEAETKTKDEMAAAEEK.V | C[+57] | 462.209 | 5 | 2307.018 | 2307.023 | 0 | −2.1 | 308 | 329.4 | 329.4 | 329.4 | 4.77 |
| 1357 | K.TKDEMAAAEEKVGK.N | | 502.920 | 3 | 1506.746 | 1506.747 | 0 | −0.3 | 317 | 698.2 | 585.0 | 585.0 | 10.51 |
| 1493 | K.VGKN[+0.984]EQGEPEDLKKPEGK.N | N[+1] | 496.506 | 4 | 1983.003 | 1983.003 | 0 | 0.2 | 328 | 387.9 | 387.9 | 101.7 | 7.35 |
| 2203 | K.NQEADVQEVK.A | | 580.283 | 2 | 1159.558 | 1159.559 | 0 | −0.5 | 360 | 426.8 | 426.8 | 426.8 | 6.57 |
| 1716 | R.GGEEFGESKWDSNIC[+57.021]ELHYTR.D | C[+57] | 629.280 | 4 | 2514.098 | 2514.099 | 0 | −0.3 | 404 | 497.4 | 497.4 | 497.4 | 7.61 |
| 1701 | K.WDSNIC[+57.021]ELHYTR.D | C[+57] | 531.909 | 3 | 1593.712 | 1593.712 | 0 | 0.2 | 413 | 409.4 | 409.4 | 409.4 | 7.35 |
| 1701 | K.WDSNIC[+57.021]ELHYTR.D | C[+57] | 531.908 | 3 | 1593.711 | 1593.712 | 0 | 0.5 | 413 | 447.6 | 447.6 | 447.6 | 6.99 |
| 994 | K.SSLLGSGDWHQYYDIVYMKPHGR.L | | 678.078 | 4 | 2709.288 | 2709.288 | 0 | 0.1 | 484 | 542.1 | 542.1 | 542.1 | 9.25 |
| 994 | K.SSLLGSGDWHQYYDIVYMKPHGR.L | | 542.663 | 5 | 2709.288 | 2709.288 | 0 | 0.0 | 484 | 504.7 | 504.7 | 504.7 | 9.07 |
| 2034 | R.LQKVMNHITDGPRK.D | | 409.979 | 4 | 1636.895 | 1636.895 | 0 | 0.1 | 507 | 386.7 | 386.7 | 386.7 | 6.80 |
| 860 | K.VMNHITDGPRKDLVK.G | | 431.489 | 4 | 1722.934 | 1722.932 | 0 | 1.4 | 510 | 410.4 | 410.4 | 410.4 | 7.20 |
| 860 | K.VMNHITDGPRKDLVK.G | | 574.982 | 3 | 1722.932 | 1722.932 | 0 | 0.0 | 510 | 554.7 | 554.7 | 554.7 | 6.94 |
| 1526 | K.VM[+15.995]NHITDGPRKDLVK.G | M[+16] | 435.487 | 4 | 1738.927 | 1738.927 | 0 | 0.3 | 510 | 363.8 | 363.8 | 363.8 | 6.80 |
| 860 | K.VMNHITDGPRKDLVK.G | | 431.488 | 4 | 1722.932 | 1722.932 | 0 | −0.2 | 510 | 397.8 | 397.8 | 397.8 | 6.21 |
| 860 | K.VMNHITDGPRKDLVK.G | | 574.982 | 3 | 1722.932 | 1722.932 | 0 | −0.1 | 510 | 518.0 | 518.0 | 518.0 | 6.17 |
| 860 | K.VMNHITDGPRKDLVK.G | | 574.982 | 3 | 1722.932 | 1722.932 | 0 | −0.1 | 510 | 504.4 | 504.4 | 504.4 | 6.17 |
| 860 | K.VMNHITDGPRKDLVK.G | | 431.489 | 4 | 1722.933 | 1722.932 | 0 | 0.5 | 510 | 353.5 | 353.5 | 353.5 | 5.76 |
| 860 | K.VMNHITDGPRKDLVK.G | | 861.970 | 2 | 1722.932 | 1722.932 | 0 | 0.1 | 510 | 368.7 | 368.7 | 368.7 | 5.19 |
| 1526 | K.VM[+15.995]NHITDGPRKDLVK.G | M[+16] | 580.314 | 3 | 1738.926 | 1738.927 | 0 | −0.3 | 510 | 392.8 | 392.8 | 392.8 | 4.60 |
| 1834 | K.RYLWQHLKK.H | | 424.584 | 3 | 1271.738 | 1271.737 | 0 | 0.9 | 588 | 315.6 | 315.6 | 315.6 | 3.93 |
| 2363 | R.YLWQHLK.K | | 494.274 | 2 | 987.541 | 987.541 | 0 | 0.4 | 589 | 477.1 | 477.1 | 477.1 | 6.80 |
| 907 | R.YLWQHLKK.H | | 558.322 | 2 | 1115.636 | 1115.636 | 0 | −0.1 | 589 | 418.1 | 418.1 | 418.1 | 5.65 |
| 907 | R.YLWQHLKK.H | | 558.322 | 2 | 1115.636 | 1115.636 | 0 | 0.0 | 589 | 310.9 | 310.9 | 310.9 | 3.74 |
| 1704 | K.STDFLPVDC[+57.021]PVR.S | C[+57] | 703.343 | 2 | 1405.678 | 1405.678 | 0 | 0.0 | 606 | 449.4 | 449.4 | 449.4 | 8.05 |
| 1704 | K.STDFLPVDC[+57.021]PVR.S | C[+57] | 469.231 | 3 | 1405.678 | 1405.678 | 0 | −0.1 | 606 | 456.2 | 401.8 | 401.8 | 7.05 |
| 812 | R.DLSHILGIPQSWR.L | | 507.944 | 3 | 1521.817 | 1521.817 | 0 | −0.3 | 661 | 398.4 | 398.4 | 398.4 | 6.83 |
| 1678 | R.TYTWLGALPVLHC[+57.021]C[+57.021]MELAPR.H | C[+57]* | 797.061 | 3 | 2389.169 | 2388.166 | 1 | −0.3 | 688 | 472.5 | 472.5 | 472.5 | 7.25 |
| | | 2 | | | | | | | | | | | |
| 2001 | R.HKDAWRQPEDTWAALEGLSFSPFR.E | | 569.683 | 5 | 2844.386 | 2844.385 | 0 | 0.2 | 708 | 335.3 | 279.7 | 279.7 | 5.43 |
| 2002 | K.DAWRQPEDTWAALEGLSFSPFR.E | | 860.416 | 3 | 2579.233 | 2579.231 | 0 | 0.4 | 710 | 369.3 | 369.3 | 369.3 | 7.02 |
| 2002 | K.DAWRQPEDTWAALEGLSFSPFR.E | | 860.416 | 3 | 2579.233 | 2579.231 | 0 | 0.4 | 710 | 338.6 | 338.6 | 338.6 | 5.70 |
| 1133 | R.QPEDTWAALEGLSFSPFREQMLDTSSLLQFMR.E | | 933.453 | 4 | 3730.792 | 3730.788 | 0 | 1.0 | 714 | 798.7 | 798.7 | 798.7 | 12.47 |
| 885 | R.QPEDTWAALEGLSFSPFR.E | | 1025.998 | 2 | 2050.988 | 2050.987 | 0 | 0.6 | 714 | 485.0 | 485.0 | 485.0 | 9.07 |
| 1133 | R.QPEDTWAALEGLSFSPFREQMLDTSSLLQFMR.E | | 1244.269 | 3 | 3730.792 | 3730.788 | 0 | 1.2 | 714 | 570.2 | 570.2 | 570.2 | 8.99 |
| 1133 | R.QPEDTWAALEGLSFSPFREQMLDTSSLLQFMR.E | | 933.454 | 4 | 3730.792 | 3730.788 | 0 | 1.1 | 714 | 634.3 | 634.3 | 634.3 | 8.88 |
| 885 | R.QPEDTWAALEGLSFSPFR.E | | 684.334 | 3 | 2050.988 | 2050.987 | 0 | 0.5 | 714 | 456.1 | 456.1 | 456.1 | 8.39 |
| 1133 | R.QPEDTWAALEGLSFSPFREQMLDTSSLLQFMR.E | | 1244.270 | 3 | 3730.794 | 3730.788 | 0 | 1.7 | 714 | 495.0 | 495.0 | 495.0 | 7.19 |
| 885 | R.QPEDTWAALEGLSFSPFR.E | | 684.334 | 3 | 2050.988 | 2050.987 | 0 | 0.3 | 714 | 340.1 | 340.1 | 340.1 | 5.84 |
| 1616 | R.QPEDTWAALEGLSFSPFREQM[+15.995]LDTSSLLQFMR.E | M[+16] | 937.452 | 4 | 3746.787 | 3746.783 | 0 | 1.0 | 714 | 451.6 | 241.5 | 109.8 | 4.60 |
| 949 | R.EQMLDTSSLLQFMR.E | | 849.913 | 2 | 1698.819 | 1698.819 | 0 | 0.1 | 732 | 615.9 | 615.9 | 615.9 | 10.72 |
| 949 | R.EQMLDTSSLLQFMR.E | | 566.945 | 3 | 1698.820 | 1698.819 | 0 | 0.7 | 732 | 569.7 | 569.7 | 569.7 | 9.20 |
| 1558 | R.EQM[+15.995]LDTSSLLQFMR.E | M[+16] | 572.276 | 3 | 1714.815 | 1714.814 | 0 | 0.4 | 732 | 388.9 | 388.9 | 388.9 | 6.90 |
| 1558 | R.EQM[+15.995]LDTSSLLQFMR.E | M[+16] | 857.911 | 2 | 1714.815 | 1714.814 | 0 | 0.9 | 732 | 333.1 | 333.1 | 252.8 | 5.91 |
| 1860 | R.EKQHLLSIDEPLFR.S | | 575.649 | 3 | 1724.934 | 1724.933 | 0 | 0.3 | 746 | 386.3 | 386.3 | 386.3 | 7.14 |
| 1860 | R.EKQHLLSIDEPLFR.S | | 431.989 | 4 | 1724.933 | 1724.933 | 0 | −0.1 | 746 | 311.2 | 311.2 | 311.2 | 5.35 |
| 2190 | K.QHLLSIDEPLFR.S | | 489.937 | 3 | 1467.796 | 1467.795 | 0 | 0.1 | 748 | 409.0 | 409.0 | 409.0 | 7.53 |
| 1226 | R.DETGNNSVQTVFQGTLAATKR.W | | 746.377 | 3 | 2237.115 | 2237.116 | 0 | 0.2 | 892 | 671.3 | 671.3 | 671.3 | 11.12 |
| 867 | R.LVEIQFPAEHGWKESLLGDMEWR.L | | 693.348 | 4 | 2770.368 | 2770.366 | 0 | 1.0 | 942 | 436.5 | 436.5 | 436.5 | 7.81 |
| 1882 | R.LVEIQFPAEHGWK.E | | 518.609 | 3 | 1553.811 | 1553.811 | 0 | 0.2 | 942 | 335.7 | 335.7 | 335.7 | 5.91 |
| 996 | K.GLEDSVAKTFEK.C | | 441.898 | 3 | 1323.679 | 1323.679 | 0 | 0.1 | 986 | 517.2 | 517.2 | 517.2 | 7.94 |
| 972 | K.FGIVLSAVITK.S | | 574.358 | 2 | 1147.709 | 1147.709 | 0 | 0.3 | 1025 | 508.1 | 508.1 | 508.1 | 8.21 |
| 913 | R.TADNFDDILKHLLTLADVK.H | | 714.721 | 3 | 2142.148 | 2142.144 | 0 | 1.6 | 1040 | 666.2 | 666.2 | 666.2 | 11.70 |
| 913 | R.TADNFDDILKHLLTLADVK.H | | 536.292 | 4 | 2142.145 | 2142.144 | 0 | 0.2 | 1040 | 539.9 | 539.9 | 539.9 | 8.97 |
| 1060 | K.HLLTLADVK.H | | 505.306 | 2 | 1009.604 | 1009.604 | 0 | 0.1 | 1050 | 523.6 | 523.6 | 523.6 | 8.10 |
| 1724 | R.LC[+57.021]GTDEKILANVTEDAKR.L | C[+57] | 678.349 | 3 | 2033.033 | 2033.033 | 0 | 0.1 | 1063 | 592.3 | 592.3 | 592.3 | 9.36 |
| 1724 | R.LC[+57.021]GTDEKILANVTEDAKR.L | C[+57] | 509.014 | 4 | 2033.033 | 2033.033 | 0 | −0.1 | 1063 | 571.3 | 571.3 | 571.3 | 9.36 |
| 951 | K.ILANVTEDAKR.L | | 615.346 | 2 | 1229.684 | 1229.685 | 0 | −0.4 | 1070 | 606.6 | 606.6 | 606.6 | 9.26 |
| 951 | K.ILANVTEDAKR.L | | 410.566 | 3 | 1229.685 | 1229.685 | 0 | 0.2 | 1070 | 507.9 | 507.9 | 507.9 | 7.94 |
| 829 | K.ILANVTEDAK.R | | 537.295 | 2 | 1073.583 | 1073.584 | 0 | 0.7 | 1070 | 509.0 | 381.6 | 381.6 | 7.62 |
| 951 | K.ILANVTEDAKR.L | | 410.567 | 3 | 1229.685 | 1229.685 | 0 | 0.3 | 1070 | 404.3 | 404.3 | 404.3 | 7.55 |
| 951 | K.ILANVTEDAKR.L | | 410.567 | 3 | 1229.685 | 1229.685 | 0 | 0.2 | 1070 | 459.3 | 394.0 | 394.0 | 7.49 |
| 951 | K.ILANVTEDAKR.L | | 410.567 | 3 | 1229.686 | 1229.685 | 0 | 0.6 | 1070 | 436.4 | 436.4 | 436.4 | 7.07 |
| 829 | K.ILANVTEDAK.R | | 537.295 | 2 | 1073.584 | 1073.584 | 0 | 0.2 | 1070 | 467.1 | 350.8 | 350.8 | 6.23 |
| 1772 | K.RLIAVADSVLTK.V | | 429.266 | 3 | 1285.784 | 1285.784 | 0 | 0.2 | 1080 | 496.1 | 496.1 | 496.1 | 8.21 |
| 2210 | R.LIAVADSVLTK.V | | 565.345 | 2 | 1129.682 | 1129.683 | 0 | −0.2 | 1081 | 418.2 | 418.2 | 418.2 | 7.24 |
| 2210 | R.LIAVADSVLTK.V | | 565.345 | 2 | 1129.683 | 1129.683 | 0 | 0.0 | 1081 | 436.6 | 332.3 | 332.3 | 6.30 |
| 2210 | R.LIAVADSVLTK.V | | 565.345 | 2 | 1129.682 | 1129.683 | 0 | −0.2 | 1081 | 458.7 | 342.3 | 342.3 | 5.94 |
| 1220 | K.VVGDLLSGTILVGQLELIIK.H | | 694.093 | 3 | 2080.264 | 2080.263 | 0 | 0.7 | 1092 | 490.2 | 490.2 | 490.2 | 8.55 |
| 1973 | K.HKNQFLDIWQLR.E | | 533.291 | 3 | 1597.860 | 1597.860 | 0 | −0.1 | 1112 | 404.5 | 404.5 | 404.5 | 7.26 |
| 2135 | K.NQFLDIWQLR.E | | 666.857 | 2 | 1332.707 | 1332.706 | 0 | 0.5 | 1114 | 488.1 | 488.1 | 488.1 | 8.21 |
| 2369 | K.NQFLDIWQLREK.S | | 530.619 | 3 | 1589.843 | 1589.844 | 0 | 0.1 | 1114 | 465.3 | 465.3 | 465.3 | 7.49 |
| 2135 | K.NQFLDIWQLR.E | | 444.907 | 3 | 1332.706 | 1332.706 | 0 | 0.4 | 1114 | 416.5 | 416.5 | 416.5 | 7.02 |
| 1729 | K.SLSPQDEQC[+57.021]AVEEALDWR.R | C[+57] | 1066.983 | 2 | 2132.958 | 2132.955 | 0 | 1.4 | 1126 | 582.7 | 582.7 | 582.7 | 9.98 |
| 1689 | K.SLSPQDEQC[+57.021]AVEEALDWRREELLLLK.K | C[+57] | 782.899 | 4 | 3128.575 | 3127.573 | 1 | −0.2 | 1126 | 318.9 | 318.9 | 318.9 | 4.15 |
| 989 | R.EELLLLKK.E | | 493.318 | 2 | 985.629 | 985.629 | 0 | −0.2 | 1145 | 514.1 | 514.1 | 514.1 | 7.94 |
| 2221 | R.EELLLLK.K | | 429.271 | 2 | 857.535 | 857.534 | 0 | 0.3 | 1145 | 458.6 | 458.6 | 458.6 | 7.76 |
| 989 | R.EELLLLKK.E | | 493.318 | 2 | 985.629 | 985.629 | 0 | 0.1 | 1145 | 536.2 | 536.2 | 536.2 | 7.64 |
| 1703 | R.C[+57.021]VDSLLK.M | C[+57] | 417.723 | 2 | 834.439 | 834.439 | 0 | 0.0 | 1156 | 456.4 | 456.4 | 456.4 | 7.38 |
| 1703 | R.C[+57.021]VDSLLK.M | C[+57] | 417.723 | 2 | 834.439 | 834.439 | 0 | 0.1 | 1156 | 451.1 | 451.1 | 451.1 | 7.38 |
| 926 | K.HLIQVDFGVLAVR.H | | 489.621 | 3 | 1466.848 | 1466.848 | 0 | 0.1 | 1169 | 382.1 | 382.1 | 382.1 | 7.42 |
| 1160 | R.HSQDLSSKRLNDTVTVR.L | | 489.762 | 4 | 1956.026 | 1956.026 | 0 | 0.1 | 1182 | 564.5 | 564.5 | 564.5 | 9.36 |
| 1160 | R.HSQDLSSKRLNDTVTVR.L | | 489.762 | 4 | 1956.026 | 1956.026 | 0 | 0.1 | 1182 | 384.6 | 384.6 | 384.6 | 7.35 |
| 1951 | K.RLNDTVTVR.L | | 537.307 | 2 | 1073.606 | 1073.606 | 0 | −0.2 | 1190 | 353.4 | 353.4 | 353.4 | 4.83 |
| 2114 | R.LNDTVTVR.L | | 459.256 | 2 | 917.505 | 917.505 | 0 | 0.0 | 1191 | 414.5 | 414.5 | 414.5 | 7.15 |
| 1917 | R.LSTSSNSQRATHYHLSSQVQEMAGK.I | | 687.585 | 4 | 2747.317 | 2747.317 | 0 | 0.0 | 1199 | 408.1 | 408.1 | 408.1 | 7.33 |
| 1917 | R.LSTSSNSQRATHYHLSSQVQEMAGK.I | | 687.585 | 4 | 2747.317 | 2747.317 | 0 | 0.0 | 1199 | 310.1 | 310.1 | 310.1 | 5.61 |
| 811 | R.ATHYHLSSQVQEMAGKIDLLR.D | | 600.314 | 4 | 2398.235 | 2397.234 | 1 | −1.1 | 1208 | 779.6 | 779.6 | 779.6 | 13.12 |
| 811 | R.ATHYHLSSQVQEMAGKIDLLR.D | | 600.064 | 4 | 2397.235 | 2397.234 | 0 | 0.1 | 1208 | 722.0 | 722.0 | 722.0 | 12.70 |
| 1012 | R.ATHYHLSSQVQEMAGK.I | | 596.290 | 3 | 1786.855 | 1786.854 | 0 | 0.4 | 1208 | 668.6 | 668.6 | 668.6 | 11.72 |
| 1012 | R.ATHYHLSSQVQEMAGK.I | | 596.290 | 3 | 1786.855 | 1786.854 | 0 | 0.3 | 1208 | 667.3 | 667.3 | 667.3 | 11.72 |
| 1012 | R.ATHYHLSSQVQEMAGK.I | | 596.290 | 3 | 1786.855 | 1786.854 | 0 | 0.4 | 1208 | 666.6 | 666.6 | 666.6 | 11.72 |
| 1012 | R.ATHYHLSSQVQEMAGK.I | | 596.290 | 3 | 1786.854 | 1786.854 | 0 | 0.0 | 1208 | 565.3 | 565.3 | 565.3 | 9.72 |
| 1012 | R.ATHYHLSSQVQEMAGK.I | | 893.931 | 2 | 1786.855 | 1786.854 | 0 | 0.3 | 1208 | 639.1 | 639.1 | 639.1 | 9.30 |
| 811 | R.ATHYHLSSQVQEMAGKIDLLR.D | | 480.453 | 5 | 2398.237 | 2397.234 | 1 | 0.3 | 1208 | 560.4 | 560.4 | 560.4 | 9.12 |
| 1562 | R.ATHYHLSSQVQEM[+15.995]AGK.I | M[+16] | 601.621 | 3 | 1802.849 | 1802.849 | 0 | −0.1 | 1208 | 552.7 | 552.7 | 552.7 | 8.98 |
| 1562 | R.ATHYHLSSQVQEM[+15.995]AGK.I | M[+16] | 601.621 | 3 | 1802.850 | 1802.849 | 0 | 0.3 | 1208 | 493.6 | 493.6 | 493.6 | 8.80 |
| 1012 | R.ATHYHLSSQVQEMAGK.I | | 596.290 | 3 | 1786.854 | 1786.854 | 0 | 0.0 | 1208 | 406.2 | 406.2 | 406.2 | 8.42 |
| 1012 | R.ATHYHLSSQVQEMAGK.I | | 596.290 | 3 | 1786.855 | 1786.854 | 0 | 0.7 | 1208 | 518.0 | 518.0 | 518.0 | 8.21 |
| 1562 | R.ATHYHLSSQVQEM[+15.995]AGK.I | M[+16] | 601.621 | 3 | 1802.849 | 1802.849 | 0 | 0.1 | 1208 | 441.9 | 441.9 | 441.9 | 8.05 |
| 1012 | R.ATHYHLSSQVQEMAGK.I | | 447.469 | 4 | 1786.855 | 1786.854 | 0 | 0.4 | 1208 | 414.2 | 414.2 | 414.2 | 7.83 |
| 1012 | R.ATHYHLSSQVQEMAGK.I | | 447.470 | 4 | 1786.856 | 1786.854 | 0 | 1.1 | 1208 | 412.0 | 412.0 | 412.0 | 7.53 |
| 1012 | R.ATHYHLSSQVQEMAGK.I | | 447.469 | 4 | 1786.854 | 1786.854 | 0 | 0.1 | 1208 | 388.0 | 388.0 | 388.0 | 7.42 |
| 1012 | R.ATHYHLSSQVQEMAGK.I | | 447.469 | 4 | 1786.854 | 1786.854 | 0 | 0.1 | 1208 | 373.5 | 373.5 | 373.5 | 7.23 |
| 1012 | R.ATHYHLSSQVQEMAGK.I | | 447.470 | 4 | 1786.859 | 1786.854 | 0 | 2.7 | 1208 | 464.9 | 464.9 | 464.9 | 6.64 |
| 1562 | R.ATHYHLSSQVQEM[+15.995]AGK.I | M[+16] | 451.468 | 4 | 1802.849 | 1802.849 | 0 | −0.1 | 1208 | 373.1 | 359.6 | 359.6 | 6.18 |
| 1012 | R.ATHYHLSSQVQEMAGK.I | | 447.469 | 4 | 1786.854 | 1786.854 | 0 | 0.1 | 1208 | 359.9 | 359.9 | 359.9 | 6.09 |
| 1562 | R.ATHYHLSSQVQEM[+15.995]AGK.I | M[+16] | 451.468 | 4 | 1802.849 | 1802.849 | 0 | −0.2 | 1208 | 356.3 | 324.7 | 324.7 | 6.09 |
| 1562 | R.ATHYHLSSQVQEM[+15.995]AGK.I | M[+16] | 451.468 | 4 | 1802.849 | 1802.849 | 0 | 0.0 | 1208 | 374.8 | 328.4 | 328.4 | 5.99 |
| 1012 | R.ATHYHLSSQVQEMAGK.I | | 447.469 | 4 | 1786.856 | 1786.854 | 0 | 0.8 | 1208 | 340.5 | 340.5 | 340.5 | 5.91 |
| 1562 | R.ATHYHLSSQVQEM[+15.995]AGK.I | M[+16] | 451.468 | 4 | 1802.850 | 1802.849 | 0 | 0.3 | 1208 | 361.8 | 295.9 | 295.9 | 5.79 |
| 1012 | R.ATHYHLSSQVQEMAGK.I | | 447.469 | 4 | 1786.854 | 1786.854 | 0 | 0.1 | 1208 | 333.5 | 333.5 | 333.5 | 5.71 |
| 838 | R.DSHIFQLFWR.E | | 674.844 | 2 | 1348.681 | 1348.680 | 0 | 0.7 | 1229 | 554.0 | 378.9 | 378.9 | 8.39 |
| 838 | R.DSHIFQLFWR.E | | 450.231 | 3 | 1348.680 | 1348.680 | 0 | 0.1 | 1229 | 479.9 | 479.9 | 479.9 | 7.76 |
| 1299 | R.EAAEPLSEPKEDQEAAELLSEPEEESER.H | | 1047.813 | 3 | 3141.424 | 3141.423 | 0 | 0.4 | 1239 | 742.6 | 742.6 | 742.6 | 12.70 |
| 1468 | R.EAAEPLSEPKEDQEAAELLS[+79.966]EPEEESERHILELEEV | S[+80] | 1383.884 | 4 | 5532.513 | 5532.505 | 0 | 1.3 | 1239 | 709.0 | 709.0 | 21.9 | 11.20 |
| | YDYLYQPSYR.K | | | | | | | | | | | | |
| 1469 | R.EAAEPLSEPKEDQEAAELLSEPEEES[+79.966]ERHILELEEV | S[+80] | 1383.883 | 4 | 5532.509 | 5532.505 | 0 | 0.5 | 1239 | 651.6 | 651.6 | 46.7 | 10.20 |
| | YDYLYQPSYR.K | | | | | | | | | | | | |
| 1299 | R.EAAEPLSEPKEDQEAAELLSEPEEESER.H | | 1047.813 | 3 | 3141.424 | 3141.423 | 0 | 0.2 | 1239 | 580.4 | 580.4 | 580.4 | 9.70 |
| 1469 | R.EAAEPLSEPKEDQEAAELLSEPEEES[+79.966]ERHILELEEV | S[+80] | 1107.308 | 5 | 5532.510 | 5532.505 | 0 | 0.9 | 1239 | 636.3 | 636.3 | 32.1 | 9.20 |
| | YDYLYQPSYR.K | | | | | | | | | | | | |
| 1380 | R.EAAEPLSEPKEDQEAAELLSEPEEESERHILELEEVYDYLYQPS | | 1363.889 | 4 | 5452.535 | 5452.539 | 0 | −0.8 | 1239 | 653.6 | 572.1 | 572.1 | 9.01 |
| | YR.K | | | | | | | | | | | | |
| 1380 | R.EAAEPLSEPKEDQEAAELLSEPEEESERHILELEEVYDYLYQPS | | 1091.715 | 5 | 5454.546 | 5452.539 | 2 | 0.1 | 1239 | 602.4 | 549.1 | 549.1 | 8.60 |
| | YR.K | | | | | | | | | | | | |
| 1468 | R.EAAEPLSEPKEDQEAAELLS[+79.966]EPEEESERHILELEEV | S[+80] | 1107.308 | 5 | 5532.512 | 5532.505 | 0 | 1.2 | 1239 | 539.0 | 539.0 | 8.1 | 6.58 |
| | YDYLYQPSYR.K | | | | | | | | | | | | |
| 1380 | R.EAAEPLSEPKEDQEAAELLSEPEEESERHILELEEVYDYLYQPS | | 1091.314 | 5 | 5452.539 | 5452.539 | 0 | 0.0 | 1239 | 517.6 | 517.6 | 517.6 | 6.40 |
| | YR.K | | | | | | | | | | | | |
| 1897 | R.KFIKLHQDLK.S | | 423.927 | 3 | 1269.768 | 1269.768 | 0 | 0 .. | 1285 | 333.5 | 333.5 | 333.5 | 4.02 |
| 1897 | R.KFIKLHQDLK.S | | 423.928 | 3 | 1269.768 | 1269.768 | 0 | 0.3 | 1285 | 324.1 | 324.1 | 324.1 | 4.02 |
| 2956 | K.SGEVTLAEIDVIFKDFVNK.Y | | 708.713 | 3 | 2124.124 | 2124.122 | 0 | 0.6 | 1295 | 696.9 | 696.9 | 696.9 | 11.70 |
| 1873 | R.DWIKDRVEQIK.E | | 477.265 | 3 | 1429.781 | 1429.780 | 0 | 0.6 | 1334 | 357.6 | 357.6 | 357.6 | 5.09 |
| 2161 | K.DRVEQIKEYHHLHQAVHAAK.V | | 482.657 | 5 | 2409.254 | 2409.253 | 0 | 0.3 | 1338 | 436.1 | 436.1 | 436.1 | 6.05 |
| 1892 | R.VEQIKEYHHLHQAVHAAK.V | | 428.431 | 5 | 2138.127 | 2138.125 | 0 | 0.6 | 1340 | 479.1 | 479.1 | 479.1 | 7.22 |
| 1892 | R.VEQIKEYHHLHQAVHAAK.V | | 535.287 | 4 | 2138.126 | 2138.125 | 0 | 0.4 | 1340 | 424.5 | 424.5 | 424.5 | 6.39 |
| 1892 | R.VEQIKEYHHLHQAVHAAK.V | | 428.431 | 5 | 2138.126 | 2138.125 | 0 | 0.2 | 1340 | 396.1 | 396.1 | 396.1 | 5.98 |
| 1892 | R.VEQIKEYHHLHQAVHAAK.V | | 428.431 | 5 | 2138.127 | 2138.125 | 0 | 0.8 | 1340 | 336.5 | 336.5 | 336.5 | 4.66 |
| 1892 | R.VEQIKEYHHLHQAVHAAK.V | | 428.431 | 5 | 2138.127 | 2138.125 | 0 | 0.6 | 1340 | 336.8 | 336.8 | 336.8 | 4.47 |
| 2924 | K.ESLGLNGDFSVLNTLLNFTDNFDDFRR.E | | 1040.508 | 3 | 3119.508 | 3119.507 | 0 0 | 0.4 | 1364 | 644.2 | 644.2 | 644.2 | 10.11 |
| 1077 | R.ETLDQINQELIQAKK.L | | 590.992 | 3 | 1770.961 | 1770.960 | 0 | 0.6 | 1391 | 601.2 | 590.4 | 590.4 | 10.44 |
| 946 | R.ETLDQINQELIQAK.K | | 821.936 | 2 | 1642.865 | 1642.865 | 0 | −0.1 | 1391 | 476.3 | 418.1 | 418.1 | 8.05 |
| 946 | R.ETLDQINQELIQAK.K | | 548.293 | 3 | 1642.865 | 1642.865 | 0 | 0.2 | 1391 | 380.3 | 380.3 | 380.3 | 6.90 |
| 946 | R.ETLDQINQELIQAK.K | | 548.293 | 3 | 1642.864 | 1642.865 | 0 | 0.4 | 1391 | 317.8 | 317.8 | 317.8 | 4.52 |
| 1101 | K.KLLQDISEAR.C | | 586.835 | 2 | 1172.663 | 1172.663 | 0 | 0.1 | 1405 | 609.3 | 485.5 | 485.5 | 9.83 |
| 1101 | K.KLLQDISEAR.C | | 586.835 | 2 | 1172.663 | 1172.663 | 0 | 0.0 | 1405 | 321.7 | 321.7 | 321.7 | 5.71 |
| 998 | K.LLQDISEAR.C | | 522.788 | 2 | 1044.568 | 1044.568 | 0 | 0.0 | 1406 | 524.5 | 304.8 | 304.8 | 6.95 |
| 998 | K.LLQDISEAR.C | | 522.788 | 2 | 1044.568 | 1044.568 | 0 | −0.2 | 1406 | 536.1 | 315.6 | 315.6 | 6.37 |
| 998 | K.LLQDISEAR.C | | 522.788 | 2 | 1044.569 | 1044.568 | 0 | 0.1 | 1406 | 371.2 | 283.9 | 283.9 | 5.79 |
| 998 | K.LLQDISEAR.C | | 522.788 | 2 | 1044.568 | 1044.568 | 0 | −0.2 | 1406 | 386.4 | 253.2 | 253.2 | 4.92 |
| 2286 | R.EALGGINELK.V | | 522.290 | 2 | 1043.573 | 1043.573 | 0 | 0.2 | 1433 | 447.3 | 447.3 | 447.3 | 7.76 |
| 2191 | K.ALDKDQYLPR.K | | 406.887 | 3 | 1218.648 | 1218.648 | 0 | 0.0 | 1498 | 529.7 | 529.7 | 529.7 | 8.12 |
| 1029 | K.ALDKDQYLPRK.L | | 449.586 | 3 | 1346.743 | 1346.743 | 0 | −0.1 | 1498 | 489.3 | 489.3 | 489.3 | 7.30 |
| 2191 | K.ALDKDQYLPR.K | | 609.828 | 2 | 1218.648 | 1218.648 | 0 | 0.4 | 1498 | 420.0 | 420.0 | 420.0 | 7.07 |
| 1029 | K.ALDKDQYLPRK.L | | 449.586 | 3 | 1346.744 | 1346.743 | 0 | 0.7 | 1498 | 381.0 | 381.0 | 381.0 | 6.61 |
| 832 | R.NLEWLKTVNESHGSVER.S | | 500.257 | 4 | 1998.004 | 1998.004 | 0 | 0.0 | 1515 | 474.8 | 474.8 | 474.8 | 8.04 |
| 1766 | R.NLEWLK.T | | 401.727 | 2 | 802.446 | 802.446 | 0 | 0.0 | 1515 | 301.2 | 199.1 | 199.1 | 4.74 |
| 1766 | R.NLEWLK.T | | 401.727 | 2 | 802.446 | 802.446 | 0 | −0.1 | 1515 | 309.8 | 97.7 | 97.7 | 4.11 |
| 2033 | K.TVNESHGSVER.S | | 405.531 | 3 | 1214.577 | 1214.576 | 0 | 0.7 | 1521 | 372.6 | 372.6 | 372.6 | 7.23 |
| 808 | R.SSLTLATAINQR.G | | 637.857 | 2 | 1274.706 | 1274.706 | 0 | 0.0 | 1532 | 685.8 | 685.8 | 685.8 | 11.72 |
| 808 | R.SSLTLATAINQR.G | | 637.857 | 2 | 1274.706 | 1274.706 | 0 | −0.1 | 1532 | 657.0 | 657.0 | 657.0 | 11.72 |
| 808 | R.SSLTLATAINQR.G | | 637.857 | 2 | 1274.707 | 1274.706 | 0 | 0.7 | 1532 | 635.4 | 635.4 | 635.4 | 10.72 |
| 808 | R.SSLTLATAINQR.G | | 637.857 | 2 | 1274.707 | 1274.706 | 0 | 0.6 | 1532 | 577.1 | 577.1 | 577.1 | 9.12 |
| 808 | R.SSLTLATAINQR.G | | 637.857 | 2 | 1274.706 | 1274.706 | 0 | 0.0 | 1532 | 503.1 | 503.1 | 503.1 | 8.21 |
| 808 | R.SSLTLATAINQR.G | | 637.857 | 2 | 1274.707 | 1274.706 | 0 | 0.3 | 1532 | 367.7 | 367.7 | 367.7 | 7.42 |
| 808 | R.SSLTLATAINQR.G | | 425.574 | 3 | 1274.707 | 1274.706 | 0 | 0.2 | 1532 | 412.9 | 412.9 | 412.9 | 7.02 |
| 847 | R.GIYVIQAPKGGQK.I | | 679.893 | 2 | 1358.779 | 1358.779 | 0 | 0.2 | 1544 | 649.8 | 649.8 | 649.8 | 9.86 |
| 813 | R.GIYVIQAPK.G | | 494.795 | 2 | 988.583 | 988.583 | 0 | 0.2 | 1544 | 417.1 | 417.1 | 417.1 | 7.53 |
| 813 | R.GIYVIQAPK.G | | 494.795 | 2 | 988.583 | 988.583 | 0 | −0.1 | 1544 | 433.3 | 433.3 | 433.3 | 7.15 |
| 847 | R.GIYVIQAPKGGQK.I | | 679.893 | 2 | 1358.779 | 1358.779 | 0 | −0.2 | 1544 | 423.7 | 423.7 | 423.7 | 6.68 |
| 813 | R.GIYVIQAPK.G | | 494.795 | 2 | 988.583 | 988.583 | 0 | 0.3 | 1544 | 347.9 | 278.7 | 278.7 | 5.61 |
| 1331 | K.ISPDTVLHLILPESPGSHEESR.E | | 604.064 | 4 | 2413.236 | 2413.236 | 0 | 0.0 | 1557 | 633.2 | 633.2 | 633.2 | 10.98 |
| 937 | K.ISPDTVLHLILPESPGSHEESREYSLEEVKELLNK.L | | 798.417 | 5 | 3988.054 | 3988.055 | 0 | −0.1 | 1557 | 398.2 | 398.2 | 398.2 | 6.63 |
| 937 | K.ISPDTVLHLILPESPGSHEESREYSLEEVKELLNK.L | | 798.417 | 5 | 3988.055 | 3988.055 | 0 | 0.0 | 1557 | 373.4 | 373.4 | 373.4 | 6.63 |
| 937 | K.ISPDTVLHLILPESPGSHEESREYSLEEVKELLNK.L | | 665.516 | 6 | 3988.058 | 3988.055 | 0 | 0.6 | 1557 | 363.5 | 363.5 | 363.5 | 6.16 |
| 937 | K.ISPDTVLHLILPESPGSHEESREYSLEEVKELLNK.L | | 798.417 | 5 | 3988.057 | 3988.055 | 0 | 0.4 | 1557 | 400.9 | 400.9 | 400.9 | 6.08 |
| 937 | K.ISPDTVLHLILPESPGSHEESREYSLEEVKELLNK.L | | 997.770 | 4 | 3988.057 | 3988.055 | 0 | 0.4 | 1557 | 511.1 | 511.1 | 511.1 | 5.43 |
| 937 | K.ISPDTVLHLILPESPGSHEESREYSLEEVKELLNK.L | | 997.770 | 4 | 3988.057 | 3988.055 | 0 | 0.4 | 1557 | 432.4 | 432.4 | 432.4 | 5.04 |
| 937 | K.ISPDTVLHLILPESPGSHEESREYSLEEVKELLNK.L | | 665.683 | 6 | 3989.059 | 3988.055 | 1 | 0.1 | 1557 | 308.6 | 308.6 | 259.7 | 4.55 |
| 937 | K.ISPDTVLHLILPESPGSHEESREYSLEEVKELLNK.L | | 570.586 | 7 | 3988.058 | 3988.055 | 0 | 0.8 | 1557 | 305.5 | 305.5 | 305.5 | 4.03 |
| 982 | R.EYSLEEVKELLNK.L | | 797.422 | 2 | 1593.837 | 1593.837 | 0 | 0.1 | 1579 | 580.8 | 389.1 | 389.1 | 9.44 |
| 982 | R.EYSLEEVKELLNK.L | | 531.950 | 3 | 1593.837 | 1593.837 | 0 | −0.3 | 1579 | 468.7 | 468.7 | 468.7 | 7.19 |
| 1718 | R.NNTEVERFSEVFC[+57.021]SVQR.L | C[+57] | 700.998 | 3 | 2100.979 | 2100.977 | 0 | 0.9 | 1602 | 513.3 | 513.3 | 513.3 | 8.20 |
| 928 | R.LSQAFIDLHSAGNMLFR.T | | 640.665 | 3 | 1919.980 | 1919.980 | 0 | 0.2 | 1619 | 496.8 | 496.8 | 496.8 | 9.07 |
| 1595 | R.LSQAFIDLHSAGNM[+15.995]LFR.T | M[+16] | 645.997 | 3 | 1935.975 | 1935.975 | 0 | 0.4 | 1619 | 416.8 | 416.8 | 416.8 | 7.80 |
| 899 | R.KQPPSDAALTMLSFIK.S | | 873.976 | 2 | 1746.946 | 1746.946 | 0 | 0.2 | 1718 | 443.1 | 443.1 | 443.1 | 8.06 |
| 899 | R.KQPPSDAALTMLSFIK.S | | 582.987 | 3 | 1746.946 | 1746.946 | 0 | −0.1 | 1718 | 401.5 | 401.5 | 401.5 | 7.53 |
| 1899 | R.KQPPSDAALTMLSFIK.S | | 582.987 | 3 | 1746.946 | 1746.946 | 0 | 0.2 | 1718 | 386.7 | 386.7 | 386.7 | 7.42 |
| 899 | R.KQPPSDAALTMLSFIK.S | | 873.976 | 2 | 1746.946 | 1746.946 | 0 | −0.2 | 1718 | 327.2 | 327.2 | 327.2 | 5.80 |
| 809 | K.QPPSDAALTMLSFIK.S | | 809.929 | 2 | 1618.851 | 1618.851 | 0 | −0.1 | 1719 | 497.0 | 497.0 | 497.0 | 8.80 |
| 809 | K.QPPSDAALTMLSFIK.S | | 809.929 | 2 | 1618.851 | 1618.851 | 0 | 0.0 | 1719 | 459.3 | 459.3 | 459.3 | 8.65 |
| 809 | K.QPPSDAALTMLSFIK.S | | 540.289 | 3 | 1618.851 | 1618.851 | 0 | −0.1 | 1719 | 452.5 | 452.5 | 452.5 | 8.13 |
| 809 | K.QPPSDAALTMLSFIK.S | | 809.930 | 2 | 1618.852 | 1618.851 | 0 | 0.6 | 1719 | 452.6 | 388.5 | 388.5 | 7.76 |
| 809 | K.QPPSDAALTMLSFIK.S | | 540.289 | 3 | 1618.851 | 1618.851 | 0 | 0.2 | 1719 | 440.7 | 440.7 | 440.7 | 7.53 |
| 809 | K.QPPSDAALTMLSFIK.S | | 809.929 | 2 | 1618.852 | 1618.851 | 0 | 0.4 | 1719 | 399.9 | 399.9 | 399.9 | 7.42 |
| 809 | K.QPPSDAALTMLSFIK.S | | 540.288 | 3 | 1618.851 | 1618.851 | 0 | −0.2 | 1719 | 419.8 | 419.8 | 419.8 | 6.43 |
| 809 | K.QPPSDAALTMLSFIK.S | | 540.288 | 3 | 1618.851 | 1618.851 | 0 | −0.2 | 1719 | 397.3 | 397.3 | 397.3 | 6.31 |
| 809 | K.QPPSDAALTMLSFIK.S | | 809.929 | 2 | 1618.851 | 1618.851 | 0 | 0.3 | 1719 | 329.4 | 329.4 | 329.4 | 6.09 |
| 809 | K.QPPSDAALTMLSFIK.S | | 809.929 | 2 | 1618.851 | 1618.851 | 0 | −0.2 | 1719 | 323.4 | 323.4 | 323.4 | 5.51 |
| 1312 | R.RVMEELPLMLLSEFSLVDKLR.I | | 840.131 | 3 | 2518.378 | 2518.377 | 0 | 0.2 | 1759 | 679.0 | 679.0 | 679.0 | 11.70 |
| 1312 | R.RVMEELPLMLLSEFSLVDKLR.I | | 630.350 | 4 | 2518.378 | 2518.377 | 0 | 0.2 | 1759 | 649.5 | 649.5 | 649.5 | 10.70 |
| 1633 | R.RVMEELPLM[+15.995]LLSEFSLVDKLR.I | M[+16] | 845.462 | 3 | 2534.373 | 2534.372 | 0 | 0.3 | 1759 | 613.2 | 613.2 | 257.5 | 10.70 |
| 1633 | R.RVMEELPLM[+15.995]LLSEFSLVDKLR.I | M[+16] | 634.349 | 4 | 2534.374 | 2534.372 | 0 | 0.7 | 1759 | 561.6 | 561.6 | 313.5 | 9.70 |
| 1003 | R.RVMEELPLMLLSEFSLVDK.L | | 750.403 | 3 | 2249.193 | 2249.192 | 0 | 0.5 | 1759 | 496.0 | 496.0 | 496.0 | 8.47 |
| 1593 | R.RVMEELPLM[+15.995]LLSEFSLVDK.L | M[+16] | 755.734 | 3 | 2265.187 | 2265.187 | 0 | 0.0 | 1759 | 431.0 | 431.0 | 317.6 | 7.61 |
| 1626 | R.VM[+15.995]EELPLMLLSEFSLVDKLR.I | M[+16] | 793.429 | 3 | 2378.272 | 2378.271 | 0 | 0.5 | 1760 | 757.8 | 757.8 | 423.1 | 13.70 |
| 862 | R.VMEELPLMLLSEFSLVDKLR.I | | 591.325 | 4 | 2362.277 | 2362.276 | 0 | 0.3 | 1760 | 761.7 | 761.7 | 761.7 | 13.19 |
| 862 | R.VMEELPLMLLSEFSLVDKLR.I | | 788.098 | 3 | 2362.278 | 2362.276 | 0 | 1.0 | 1760 | 739.2 | 739.2 | 739.2 | 12.70 |
| 862 | R.VMEELPLMLLSEFSLVDKLR.I | | 788.097 | 3 | 2362.277 | 2362.276 | 0 | 0.6 | 1760 | 725.9 | 725.9 | 725.9 | 12.70 |
| 1606 | R.VMEELPLM[+15.995]LLSEFSLVDKLR.I | M[+16] | 793.429 | 3 | 2378.273 | 2378.271 | 0 | 1.0 | 1760 | 660.1 | 660.1 | 371.0 | 11.70 |
| 1606 | R.VMEELPLM[+15.995]LLSEFSLVDKLR.I | M[+16] | 595.323 | 4 | 2378.272 | 2378.271 | 0 | 0.4 | 1760 | 674.7 | 674.7 | 368.7 | 11.19 |
| 862 | R.VMEELPLMLLSEFSLVDKLR.I | | 1181.644 | 2 | 2362.280 | 2362.276 | 0 | 1.5 | 1760 | 630.3 | 630.3 | 630.3 | 10.70 |
| 1626 | R.VM[+15.995]EELPLMLLSEFSLVDKLR.I | M[+16] | 793.428 | 3 | 2378.270 | 2378.271 | 0 | −0.2 | 1760 | 645.8 | 645.8 | 99.7 | 10.12 |
| 1606 | R.VMEELPLM[+15.995]LLSEFSLVDKLR.I | M[+16] | 793.429 | 3 | 2378.272 | 2378.271 | 0 | 0.6 | 1760 | 574.5 | 574.5 | 399.5 | 9.70 |
| 1614 | R.VMEELPLM[+15.995]LLSEFSLVDK.L | M[+16] | 703.701 | 3 | 2109.089 | 2109.086 | 0 | 1.4 | 1760 | 577.8 | 577.8 | 402.7 | 9.46 |
| 1082 | R.VMEELPLMLLSEFSLVDK.L | | 1047.051 | 2 | 2093.094 | 2093.091 | 0 | 1.4 | 1760 | 526.7 | 526.7 | 526.7 | 9.25 |
| 1614 | R.VMEELPLM[+15.995]LLSEFSLVDK.L | M[+16] | 1055.048 | 2 | 2109.088 | 2109.086 | 0 | 1.1 | 1760 | 495.7 | 495.7 | 296.6 | 9.07 |
| 1626 | R.VM[+15.995]EELPLMLLSEFSLVDKLR.I | M[+16] | 1189.639 | 2 | 2378.272 | 2378.271 | 0 | 0.3 | 1760 | 530.0 | 530.0 | 276.3 | 8.97 |
| 1606 | R.VMEELPLM[+15.995]LLSEFSLVDKLR.I | M[+16] | 1189.639 | 2 | 2378.271 | 2378.271 | 0 | 0.2 | 1760 | 467.2 | 467.2 | 313.8 | 8.64 |
| 1082 | R.VMEELPLMLLSEFSLVDK.L | | 698.369 | 3 | 2093.093 | 2093.091 | 0 | 0.9 | 1760 | 518.4 | 518.4 | 518.4 | 8.55 |
| 1606 | R.VMEELPLM[+15.995]LLSEFSLVDKLR.I | M[+16] | 793.429 | 3 | 2378.273 | 2378.271 | 0 | 1.0 | 1760 | 419.9 | 419.9 | 331.8 | 8.41 |
| 862 | R.VMEELPLMLLSEFSLVDKLR.I | | 788.097 | 3 | 2362.278 | 2362.276 | 0 | 0.7 | 1760 | 404.7 | 404.7 | 404.7 | 7.81 |
| 1529 | R.VM[+15.995]EELPLMLLSEFSLVDK.L | M[+16] | 703.701 | 3 | 2109.087 | 2109.086 | 0 | 0.6 | 1760 | 454.0 | 454.0 | 233.1 | 7.79 |
| 862 | R.VMEELPLMLLSEFSLVDKLR.I | | 788.097 | 3 | 2362.278 | 2362.276 | 0 | 0.7 | 1760 | 447.1 | 447.1 | 447.1 | 7.56 |
| 1529 | R.VM[+15.995]EELPLMLLSEFSLVDK.L | M[+16] | 703.701 | 3 | 2109.087 | 2109.086 | 0 | 0.7 | 1760 | 375.9 | 375.9 | 196.2 | 7.16 |
| 862 | R.VMEELPLMLLSEFSLVDKLR.I | | 788.098 | 3 | 2362.279 | 2362.276 | 0 | 1.1 | 1760 | 347.2 | 347.2 | 347.2 | 6.08 |
| 834 | R.IIMEQSMR.C | | 504.254 | 2 | 1007.501 | 1007.501 | 0 | 0.0 | 1780 | 504.7 | 504.7 | 504.7 | 7.73 |
| 834 | R.IIMEQSMR.C | | 504.254 | 2 | 1007.501 | 1007.501 | 0 | −0.2 | 1780 | 434.4 | 434.4 | 434.4 | 7.35 |
| 834 | R.IIMEQSMR.C | | 504.254 | 2 | 1007.501 | 1007.501 | 0 | 0.1 | 1780 | 365.5 | 365.5 | 365.5 | 7.23 |
| 834 | R.IIMEQSMR.C | | 504.254 | 2 | 1007.501 | 1007.501 | 0 | −0.1 | 1780 | 349.9 | 349.9 | 349.9 | 5.71 |
| 834 | R.IIMEQSMR.C | | 504.254 | 2 | 1007.502 | 1007.501 | 0 | 0.3 | 1780 | 327.2 | 327.2 | 327.2 | 5.71 |
| 1531 | R.IIM[+15.995]EQSMR.C | M[+16] | 512.252 | 2 | 1023.496 | 1023.496 | 0 | 0.1 | 1780 | 306.8 | 306.8 | 29.3 | 5.14 |
| 1531 | R.IIM[+15.995]EQSMR.C | M[+16] | 512.252 | 2 | 1023.496 | 1023.496 | 0 | −0.5 | 1780 | 340.7 | 340.7 | 19.1 | 4.83 |
| 851 | K.VFVTPQAPLEAIQAYLAGHYRVPK.Q | | 667.871 | 4 | 2668.463 | 2668.461 | 0 | 0.6 | 1948 | 824.7 | 824.7 | 824.7 | 14.70 |
| 851 | K.VFVTPQAPLEAIQAYLAGHYRVPK.Q | | 667.871 | 4 | 2668.462 | 2668.461 | 0 | 0.3 | 1948 | 771.8 | 771.8 | 771.8 | 13.70 |
| 851 | K.VFVTPQAPLEAIQAYLAGHYRVPK.Q | | 667.871 | 4 | 2668.461 | 2668.461 | 0 | 0.1 | 1948 | 765.5 | 765.5 | 765.5 | 13.70 |
| 1411 | K.VFVTPQAPLEAIQAYLAGHYR.V | | 586.817 | 4 | 2344.246 | 2344.245 | 0 | 0.5 | 1948 | 724.4 | 724.4 | 724.4 | 12.46 |
| 851 | K.VFVTPQAPLEAIQAYLAGHYRVPK.Q | | 534.498 | 5 | 2668.461 | 2668.461 | 0 | 0.1 | 1948 | 694.1 | 694.1 | 694.1 | 11.19 |
| 814 | K.QTLSAAAVFNDR.L | | 646.833 | 2 | 1292.660 | 1292.659 | 0 | 0.1 | 1972 | 624.7 | 514.2 | 514.2 | 10.72 |
| 814 | K.QTLSAAAVFNDR.L | | 646.833 | 2 | 1292.659 | 1292.659 | 0 | −0.3 | 1972 | 579.6 | 481.6 | 481.6 | 9.13 |
| 814 | K.QTLSAAAVFNDR.L | | 646.834 | 2 | 1292.660 | 1292.659 | 0 | 0.4 | 1972 | 471.1 | 451.2 | 451.2 | 8.05 |
| 814 | K.QTLSAAAVFNDR.L | | 646.834 | 2 | 1292.660 | 1292.659 | 0 | 0.4 | 1972 | 469.9 | 419.2 | 419.2 | 8.05 |
| 814 | K.QTLSAAAVFNDR.L | | 431.558 | 3 | 1292.660 | 1292.659 | 0 | 0.1 | 1972 | 399.9 | 399.9 | 399.9 | 6.90 |
| 814 | K.QTLSAAAVFNDR.L | | 431.558 | 3 | 1292.659 | 1292.659 | 0 | −0.4 | 1972 | 399.1 | 399.1 | 399.1 | 6.13 |
| 1480 | K.QTLSAAAVFN[+0.984]DR.L | N[+1] | 647.321 | 2 | 1293.634 | 1293.643 | 0 | −7.5 | 1972 | 319.8 | 319.8 | 319.8 | 3.81 |
| 910 | K.MQLNVKNVPLKTIR.L | | 414.251 | 4 | 1653.982 | 1653.983 | 0 | −0.6 | 2011 | 358.4 | 358.4 | 358.4 | 4.89 |
| 1244 | R.LIDPQVDESRVLGALLPFLDAQYQK.V | | 943.512 | 3 | 2828.522 | 2828.519 | 0 | 1.0 | 2025 | 497.7 | 497.7 | 497.7 | 7.91 |
| 1244 | R.LIDPQVDESRVLGALLPFLDAQYQK.V | | 707.886 | 4 | 2828.521 | 2828.519 | 0 | 0.5 | 2025 | 413.0 | 413.0 | 413.0 | 7.30 |
| 1009 | R.LIDPQVDESR.V | | 586.301 | 2 | 1171.595 | 1171.595 | 0 | 0.1 | 2025 | 476.6 | 306.3 | 306.3 | 6.32 |
| 1009 | R.LIDPQVDESR.V | | 586.301 | 2 | 1171.595 | 1171.595 | 0 | −0.6 | 2025 | 421.0 | 231.7 | 231.7 | 5.46 |
| 871 | R.VLGALLPFLDAQYQK.V | | 559.319 | 3 | 1675.942 | 1675.942 | 0 | 0.3 | 2035 | 535.9 | 535.9 | 535.9 | 8.47 |
| 843 | R.TRVPQFSFLDIFPK.V | | 565.647 | 3 | 1694.927 | 1694.927 | 0 | 0.5 | 2116 | 509.6 | 509.6 | 509.6 | 7.64 |
| 843 | R.TRVPQFSFLDIFPK.V | | 565.647 | 3 | 1694.926 | 1694.927 | 0 | −0.4 | 2116 | 422.0 | 422.0 | 422.0 | 7.57 |
| 843 | R.TRVPQFSFLDIFPK.V | | 565.647 | 3 | 1694.927 | 1694.927 | 0 | 0.1 | 2116 | 391.3 | 391.3 | 391.3 | 7.43 |
| 843 | R.TRVPQFSFLDIFPK.V | | 565.647 | 3 | 1694.927 | 1694.927 | 0 | 0.1 | 2116 | 416.4 | 416.4 | 416.4 | 7.26 |
| 843 | R.TRVPQFSFLDIFPK.V | | 565.647 | 3 | 1694.927 | 1694.927 | 0 | 0.3 | 2116 | 385.9 | 385.9 | 385.9 | 7.14 |
| 843 | R.TRVPQFSFLDIFPK.V | | 847.966 | 2 | 1694.926 | 1694.927 | 0 | −0.6 | 2116 | 423.8 | 423.8 | 423.8 | 6.68 |
| 843 | R.TRVPQFSFLDIFPK.V | | 565.647 | 3 | 1694.927 | 1694.927 | 0 | 0.5 | 2116 | 359.5 | 359.5 | 359.5 | 6.11 |
| 843 | R.TRVPQFSFLDIFPK.V | | 565.647 | 3 | 1694.928 | 1694.927 | 0 | 0.6 | 2116 | 331.2 | 331.2 | 331.2 | 5.82 |
| 843 | R.TRVPQFSFLDIFPK.V | | 565.647 | 3 | 1694.928 | 1694.927 | 0 | 0.7 | 2116 | 318.4 | 318.4 | 318.4 | 5.54 |
| 830 | R.VPQFSFLDIFPK.V | | 719.393 | 2 | 1437.779 | 1437.778 | 0 | 0.7 | 2118 | 578.8 | 578.8 | 578.8 | 9.72 |
| 830 | R.VPQFSFLDIFPK.V | | 479.931 | 3 | 1437.777 | 1437.778 | 0 | −0.3 | 2118 | 401.9 | 401.9 | 401.9 | 6.72 |
| 830 | R.VPQFSFLDIFPK.V | | 719.393 | 2 | 1437.779 | 1437.778 | 0 | 0.7 | 2118 | 320.1 | 320.1 | 320.1 | 5.91 |
| 1691 | K.VTC[+57.021]RPPKEVIDMELSALR.S | C[+57] | 529.283 | 4 | 2114.109 | 2114.110 | 0 | −0.5 | 2130 | 354.3 | 354.3 | 354.3 | 5.31 |
| 1691 | K.VTC[+57.021]RPPKEVIDMELSALR.S | C[+57] | 529.533 | 4 | 2115.112 | 2114.110 | 1 | −0.6 | 2130 | 321.9 | 321.9 | 321.9 | 5.31 |
| 2476 | K.EVIDMELSALR.S | | 638.334 | 2 | 1275.661 | 1275.661 | 0 | 0.0 | 2137 | 586.0 | 455.0 | 455.0 | 9.12 |
| 2476 | K.EVIDMELSALR.S | | 638.334 | 2 | 1275.661 | 1275.661 | 0 | −0.3 | 2137 | 483.7 | 448.4 | 448.4 | 7.33 |
| 1740 | R.SDTEPGMDLWEFC[+57.021]SETFQRPYQYLR.R | C[+57] | 1052.468 | 3 | 3155.391 | 3155.387 | 0 | 1.1 | 2148 | 758.2 | 758.2 | 758.2 | 13.98 |
| 1740 | R.SDTEPGMDLWEFC[+57.021]SETFQRPYQYLR.R | C[+57 | 1052.468 | 3 | 3155.390 | 3155.387 | 0 | 0.8 | 2148 | 673.6 | 673.6 | 673.6 | 11.98 |
| 1773 | R.FLNYQLR.D | | 477.264 | 2 | 953.520 | 953.520 | 0 | 0.0 | 2221 | 426.0 | 426.0 | 426.0 | 7.15 |
| 1773 | R.FLNYQLR.D | | 477.264 | 2 | 953.520 | 953.520 | 0 | 0.0 | 2221 | 423.2 | 423.2 | 423.2 | 6.86 |
| 1773 | R.FLNYQLR.D | | 477.264 | 2 | 953.520 | 953.520 | 0 | 0.3 | 2221 | 371.2 | 371.2 | 371.2 | 6.45 |
| 1773 | R.FLNYQLR.D | | 477.264 | 2 | 953.520 | 953.520 | 0 | −0.2 | 2221 | 345.8 | 293.0 | 293.0 | 5.51 |
| 1773 | R.FLNYQLR.D | | 477.264 | 2 | 953.520 | 953.520 | 0 | 0.0 | 2221 | 354.4 | 354.4 | 354.4 | 5.42 |
| 1773 | R.FLNYQLR.D | | 477.264 | 2 | 953.520 | 953.520 | 0 | 0.0 | 2221 | 333.2 | 333.2 | 333.2 | 5.42 |
| 1773 | R.FLNYQLR.D | | 477.264 | 2 | 953.520 | 953.520 | 0 | −0.1 | 2221 | 352.2 | 270.0 | 270.0 | 15.12 |
| 833 | R.DFATPSLHTSDQSPGK.H | | 563.269 | 3 | 1687.792 | 1687.792 | 0 | 0.3 | 2261 | 519.2 | 381.1 | 381.1 | 7.62 |
| 833 | R.DFATPSLHTSDQSPGK.H | | 563.269 | 3 | 1687.792 | 1687.792 | 0 | 0.2 | 2261 | 386.4 | 386.4 | 386.4 | 6.64 |
| 1466 | R.DFATPS[+79.966]LHTSDQSPGKHMVTMDGVREEDLAPFSLR.K | S[+80] | 791.167 | 5 | 3951.806 | 3951.804 | 0 | 0.4 | 2261 | 346.8 | 346.8 | 36.0 | 4.64 |
| 1088 | K.HMVTMDGVREEDLAPFSLR.K | | 735.026 | 3 | 2203.065 | 2203.063 | 0 | 0.5 | 2277 | 515.6 | 515.6 | 515.6 | 8.20 |
| 1883 | K.HMVTMDGVREEDLAPFSLRK.R | | 583.545 | 4 | 2331.157 | 2331.158 | 0 | 0.7 | 2277 | 442.3 | 442.3 | 442.3 | 7.70 |
| 1088 | K.HMVTMDGVREEDLAPFSLR.K | | 735.361 | 3 | 2204.068 | 2203.063 | 1 | 0.5 | 2277 | 390.0 | 390.0 | 390.0 | 7.40 |
| 2226 | K.HMVTMDGVR.E | | 523.250 | 2 | 1045.492 | 1045.492 | 0 | 0.2 | 2277 | 437.9 | 437.9 | 437.9 | 7.35 |
| 1088 | K.HMVTMDGVREEDLAPFSLR.K | | 735.026 | 3 | 2203.063 | 2203.063 | 0 | 0.0 | 2277 | 305.3 | 305.3 | 305.3 | 5.61 |
| 1883 | K.HMVTMDGVREEDLAPFSLRK.R | | 467.038 | 5 | 2331.158 | 2331.158 | 0 | 0.0 | 2277 | 314.0 | 314.0 | 314.0 | 5.26 |
| 1883 | K.HMVTMDGVREEDLAPFSLRK.R | | 778.059 | 3 | 2332.161 | 2331.158 | 1 | −0.3 | 2277 | 364.6 | 364.6 | 364.6 | 5.05 |
| 1537 | K.HMVTM[+15.995]DGVREEDLAPFSLRK.R | M[+16] | 587.794 | 4 | 2348.153 | 2347.153 | 1 | 1.5 | 2277 | 358.0 | 358.0 | 126.6 | 4.96 |
| 1883 | K.HMVTMDGVREEDLAPFSLRK.R | | 777.724 | 3 | 2331.158 | 2331.158 | 0 | −0.2 | 2277 | 316.7 | 316.7 | 316.7 | 3.26 |
| 1121 | R.EEDLAPFSLRK.R | | 435.566 | 3 | 1304.684 | 1304.685 | 0 | −0.2 | 2286 | 466.7 | 466.7 | 466.7 | 7.78 |
| 1121 | R.EEDLAPFSLRK.R | | 652.846 | 2 | 1304.685 | 1304.685 | 0 | 0.4 | 2286 | 519.9 | 519.9 | 519.9 | 7.64 |
| 1121 | R.EEDLAPFSLRK.R | | 435.901 | 3 | 1305.688 | 1304.685 | 1 | −0.2 | 2286 | 513.0 | 513.0 | 513.0 | 6.86 |
| 930 | R.EEDLAPFSLR.K | | 588.798 | 2 | 1176.590 | 1176.590 | 0 | 0.0 | 2286 | 504.6 | 284.8 | 284.8 | 6.77 |
| 930 | R.EEDLAPFSLR.K | | 588.798 | 2 | 1176.589 | 1176.590 | 0 | −0.2 | 2286 | 467.7 | 337.0 | 337.0 | 6.23 |
| 1885 | R.KRWESEPHPYVFFNDDHTTMTFIGFHLQPNINGSVDAISHLTGK.V | | 727.357 | 7 | 5085.455 | 5083.458 | 2 | 2.1 | 2296 | 382.4 | 382.4 | 54.8 | 4.95 |
| 1885 | R.KRWESEPHPYVFFNDDHTTMTFIGFHLQPNINGSVDAISHLTGK.V | | 727.216 | 7 | 5084.470 | 5083.458 | 1 | 1.6 | 2296 | 319.3 | 319.3 | 14.3 | 3.96 |
| 1885 | R.KRWESEPHPYVFFNDDHTTMTFIGFHLQPNINGSVDAISHLTGK.V | | 727.069 | 7 | 5083.442 | 5083.458 | 0 | −3.2 | 2296 | 312.5 | 312.5 | 312.5 | 2.55 |
| 1186 | K.RWESEPHPYVFFNDDHTTMTFIGFHLQPNINGSVDAISHLTGK.V | | 826.900 | 6 | 4956.361 | 4955.364 | 1 | −1.2 | 2297 | 629.6 | 607.6 | 47.4 | 8.64 |
| 840 | R.WESEPHPYVFFNDDHTTMTFIGFHLQPNINGSVDAISHLTGK.V | | 960.859 | 5 | 4800.265 | 4799.262 | 1 | −0.2 | 2298 | 928.3 | 928.3 | 174.3 | 15.95 |
| 840 | R.WESEPHPYVFFNDDHTTMTFIGFHLQPNINGSVDAISHLTGK.V | | 960.660 | 5 | 4799.270 | 4799.262 | 0 | 1.6 | 2298 | 746.1 | 746.1 | 746.1 | 11.82 |
| 840 | R.WESEPHPYVFFNDDHTTMTFIGFHLQPNINGSVDAISHLTGK.V | | 800.717 | 6 | 4799.268 | 4799.262 | 0 | 1.1 | 2298 | 599.2 | 599.2 | 599.2 | 8.82 |
| 840 | R.WESEPHPYVFFNDDHTTMTFIGFHLQPNINGSVDAISHLTGK.V | | 800.714 | 6 | 4799.250 | 4799.262 | 0 | 2.7 | 2298 | 566.5 | 566.5 | 566.5 | 7.34 |
| 1513 | R.WESEPHPYVFFNDDHTTMTFIGFHLQPNIN[+0.984]GSVDAIS | N[+1] | 800.881 | 6 | 4800.247 | 4800.246 | 0 | 0.2 | 2298 | 550.2 | 550.2 | 64.6 | 7.01 |
| | HLTGK.V | | | | | | | | | | | | |
| 840 | R.WESEPHPYVFFNDDHTTMTFIGFHLQPNINGSVDAISHLTGK.V | | 800.717 | 6 | 4799.262 | 4799.262 | 0 | 0.0 | 2298 | 311.4 | 311.4 | 311.4 | 4.24 |
| 1068 | R.DLYQGLLLQRVPFNVDFDKLPR.H | | 882.819 | 3 | 2646.442 | 2646.440 | 0 | 0.8 | 2349 | 850.6 | 850.6 | 850.6 | 15.35 |
| 1068 | R.DLYQGLLLQRVPFNVDFDKLPR.H | | 662.366 | 4 | 2646.441 | 2646.440 | 0 | 0.4 | 2349 | 606.2 | 606.2 | 606.2 | 10.36 |
| 1068 | R.DLYQGLLLQRVPFNVDFDKLPR.H | | 662.366 | 4 | 2646.441 | 2646.440 | 0 | 0.3 | 2349 | 568.8 | 568.8 | 568.8 | 9.36 |
| 984 | R.DLYQGLLLQR.V | | 609.846 | 2 | 1218.684 | 1218.684 | 0 | 0.2 | 2349 | 560.3 | 560.3 | 560.3 | 9.12 |
| 1068 | R.DLYQGLLLQRVPFNVDFDKLPR.H | | 530.094 | 5 | 2646.441 | 2646.440 | 0 | 0.4 | 2349 | 541.7 | 541.7 | 541.7 | 8.11 |
| 1068 | R.DLYQGLLLQRVPFNVDFDKLPR.H | | 882.819 | 3 | 2646.442 | 2646.440 | 0 | 0.6 | 2349 | 541.5 | 541.5 | 541.5 | 8.03 |
| 984 | R.DLYQGLLLQR.V | | 406.900 | 3 | 1218.684 | 1218.684 | 0 | 0.0 | 2349 | 534.5 | 534.5 | 534.5 | 7.87 |
| 984 | R.DLYQGLLLQR.V | | 609.846 | 2 | 1218.684 | 1218.684 | 0 | 0.2 | 2349 | 398.5 | 398.5 | 398.5 | 7.23 |
| 1495 | R.DLYQGLLLQRVPFN[+0.984]VDFDKLPR.H | N[+1] | 883.152 | 3 | 2647.441 | 2647.424 | 0 | 6.4 | 2349 | 433.1 | 433.1 | 223.2 | 5.24 |
| 841 | R.VPFNVDFDKLPR.H | | 482.929 | 3 | 1446.774 | 1446.774 | 0 | −0.1 | 2359 | 605.0 | 605.0 | 605.0 | 10.44 |
| 841 | R.VPFNVDFDKLPR.H | | 482.929 | 3 | 1446.774 | 1446.774 | 0 | −0.2 | 2359 | 579.3 | 579.3 | 579.3 | 9.44 |
| 841 | R.VPFNVDFDKLPR.H | | 723.891 | 2 | 1446.775 | 1446.774 | 0 | 0.3 | 2359 | 494.6 | 494.6 | 494.6 | 7.94 |
| 841 | R.VPFNVDFDKLPR.H | | 723.892 | 2 | 1446.776 | 1446.774 | 0 | 1.4 | 2359 | 459.9 | 459.9 | 459.9 | 7.78 |
| 841 | R.VPFNVDFDKLPR.H | | 482.929 | 3 | 1446.774 | 1446.774 | 0 | −0.2 | 2359 | 430.6 | 430.6 | 430.6 | 7.55 |
| 841 | R.VPFNVDFDKLPR.H | | 482.930 | 3 | 1446.774 | 1446.774 | 0 | 0.1 | 2359 | 404.8 | 404.8 | 404.8 | 7.26 |
| 841 | R.VPFNVDFDKLPR.H | | 723.891 | 2 | 1446.774 | 1446.774 | 0 | −0.1 | 2359 | 391.4 | 391.4 | 391.4 | 7.14 |
| 2022 | R.VPFNVDFDK.L | | 540.772 | 2 | 1080.536 | 1080.536 | 0 | −0.1 | 2359 | 374.7 | 374.7 | 374.7 | 6.74 |
| 841 | R.VPFNVDFDKLPR.H | | 482.929 | 3 | 1446.774 | 1446.774 | 0 | 0.3 | 2359 | 367.2 | 367.2 | 367.2 | 6.37 |
| 1748 | R.LC[+57.021]LTLGIPQATDPDKTYELTTDNMLK.I | C[+57 | 984.496 | 3 | 2951.475 | 2951.474 | 0 | 0.2 | 2377 | 772.4 | 772.4 | 772.4 | 13.70 |
| 1798 | K.ILAIEMR.F | | 423.249 | 2 | 845.491 | 845.491 | 0 | 0.1 | 2403 | 410.4 | 410.4 | 410.4 | 7.35 |
| 1570 | K.ILAIEM[+15.995]R.F | M[+16] | 431.247 | 2 | 861.486 | 861.486 | 0 | −0.1 | 2403 | 372.2 | 372.2 | 372.2 | 6.74 |
| 1798 | K.ILAIEMR.F | | 423.250 | 2 | 845.492 | 845.491 | 0 | 0.3 | 2403 | 416.2 | 273.5 | 273.5 | 5.62 |
| 1798 | K.ILAIEMR.F | | 845.491 | 1 | 845.491 | 845.491 | 0 | −0.7 | 2403 | 386.7 | 239.2 | 239.2 | 4.96 |
| 1798 | K.ILAIEMR.F | | 423.751 | 2 | 846.495 | 845.491 | 1 | 0.7 | 2403 | 306.2 | 306.2 | 306.2 | 3.75 |
| 1781 | K.FLSDLRR.G | | 453.761 | 2 | 906.515 | 906.516 | 0 | −0.2 | 2432 | 401.0 | 401.0 | 401.0 | 6.58 |
| 1781 | K.FLSDLRR.G | | 453.762 | 2 | 906.516 | 906.516 | 0 | 0.2 | 2432 | 346.0 | 218.6 | 218.6 | 4.85 |
| 1781 | K.FLSDLRR.G | | 453.761 | 2 | 906.515 | 906.516 | 0 | −0.2 | 2432 | 300.8 | 300.8 | 300.8 | 4.83 |
| 1781 | K.FLSDLRR.G | | 453.762 | 2 | 906.516 | 906.516 | 0 | 0.2 | 2432 | 332.5 | 257.6 | 257.6 | 4.81 |
| 2259 | R.GGTNADTIKLVK.V | | 608.848 | 2 | 1216.689 | 1216.690 | 0 | −0.2 | 2439 | 632.9 | 476.7 | 476.7 | 10.44 |
| 1506 | R.GGTN[+0.984]ADTIKLVK.V | N[+1] | 406.563 | 3 | 1217.674 | 1217.674 | 0 | −0.1 | 2439 | 525.1 | 427.5 | 427.5 | 8.12 |
| 2259 | R.GGTNADTIKLVK.V | | 406.235 | 3 | 1216.689 | 1216.690 | 0 | 0.3 | 2439 | 496.8 | 496.8 | 496.8 | 7.35 |
| 2259 | R.GGTNADTIKLVK.V | | 609.350 | 2 | 1217.692 | 1216.690 | 1 | −1.0 | 2439 | 424.5 | 424.5 | 424.5 | 4.62 |
| 866 | K.VHGGTTADMIYSR.V | | 704.338 | 2 | 1407.668 | 1407.669 | 0 | −0.1 | 2451 | 717.7 | 717.7 | 717.7 | 12.72 |
| 1538 | K.VHGGTTADM[+15.995]IYSR.V | M[+16] | 712.336 | 2 | 1423.664 | 1423.663 | 0 | 0.3 | 2451 | 571.8 | 571.8 | 571.8 | 9.72 |
| 866 | K.VHGGTTADMIYSR.V | | 469.894 | 3 | 1407.668 | 1407.669 | 0 | −0.3 | 2451 | 476.1 | 476.1 | 476.1 | 8.06 |
| 866 | K.VHGGTTADMIYSR.V | | 704.338 | 2 | 1407.668 | 1407.669 | 0 | −0.5 | 2451 | 454.7 | 454.7 | 454.7 | 8.06 |
| 1538 | K.VHGGTTADM[+15.995]IYSR.V | M[+16] | 475.226 | 3 | 1423.663 | 1423.663 | 0 | −0.1 | 2451 | 407.7 | 407.7 | 407.7 | 7.53 |
| 1538 | K.VHGGTTADM[+15.995]IYSR.V | M[+16] | 475.226 | 3 | 1423.663 | 1423.663 | 0 | −0.4 | 2451 | 459.4 | 459.4 | 459.4 | 7.47 |
| 1538 | K.VHGGTTADM[+15.995]IYSR.V | M[+16] | 475.226 | 3 | 1423.663 | 1423.663 | 0 | 0.0 | 2451 | 393.5 | 393.5 | 393.5 | 7.42 |
| 1538 | K.VHGGTTADM[+15.995]IYSR.V | M[+16] | 475.226 | 3 | 1423.664 | 1423.663 | 0 | 0.0 | 2451 | 401.6 | 401.6 | 401.6 | 7.35 |
| 866 | K.VHGGTTADMIYSR.V | | 469.894 | 3 | 1407.668 | 1407.669 | 0 | −0.2 | 2451 | 421.8 | 421.8 | 421.8 | 7.24 |
| 1538 | K.VHGGTTADM[+15.995]IYSR.V | M[+16] | 475.226 | 3 | 1423.664 | 1423.663 | 0 0 | 0.0 | 2451 | 391.0 | 391.0 | 391.0 | 7.23 |
| 866 | K.VHGGTTADMIYSR.V | | 469.894 | 3 | 1407.668 | 1407.669 | 0 | 0.2 | 2451 | 458.5 | 458.5 | 458.5 | 7.18 |
| 866 | K.VHGGTTADMIYSR.V | | 469.894 | 3 | 1407.668 | 1407.669 | 0 | −0.3 | 2451 | 413.7 | 413.7 | 413.7 | 6.95 |
| 1538 | K.VHGGTTADM[+15.995]IYSR.V | M[+16] | 712.335 | 2 | 1423.663 | 1423.663 | 0 | 0.0 | 2451 | 338.5 | 338.5 | 338.5 | 6.38 |
| 1538 | K.VHGGTTADM[+15.995]IYSR.V | M[+16] | 712.336 | 2 | 1423.664 | 1423.663 | 0 | 0.4 | 2451 | 344.3 | 344.3 | 344.3 | 6.09 |
| 866 | K.VHGGTTADMIYSR.V | | 704.334 | 2 | 1407.660 | 1407.669 | 0 | −6.2 | 2451 | 376.2 | 376.2 | 376.2 | 5.71 |
| 1538 | K.VHGGTTADM[+15.995]IYSR.V | M[+16] | 475.226 | 3 | 1423.664 | 1423.663 | 0 | 0.4 | 2451 | 346.1 | 346.1 | 346.1 | 5.71 |
| 866 | K.VHGGTTADMIYSR.V | | 469.894 | 3 | 1407.667 | 1407.669 | 0 | 0.9 | 2451 | 353.3 | 353.3 | 353.3 | 5.51 |
| 866 | K.VHGGTTADMIYSR.V | | 469.894 | 3 | 1407.668 | 1407.669 | 0 | −0.3 | 2451 | 351.5 | 351.5 | 351.5 | 5.51 |
| 1538 | K.VHGGTTADM[+15.995]IYSR.V | M[+16] | 475.226 | 3 | 1423.663 | 1423.663 | 0 | −0.3 | 2451 | 331.7 | 331.7 | 331.7 | 5.13 |
| 866 | K.VHGGTTADMIYSR.V | | 469.894 | 3 | 1407.668 | 1407.669 | 0 | 0.4 | 2451 | 303.4 | 207.9 | 207.9 | 4.79 |
| 1538 | K.VHGGTTADM[+15.995]IYSR.V | M[+16] | 475.226 | 3 | 1423.663 | 1423.663 | 0 | 0.4 | 2451 | 314.8 | 314.8 | 314.8 | 4.55 |
| 1926 | R.VREAENVAFANK.D | | 674.354 | 2 | 1347.701 | 1347.702 | 0 | 0.1 | 2464 | 491.6 | 491.6 | 491.6 | 7.64 |
| 1926 | R.VREAENVAFANK.D | | 449.905 | 3 | 1347.701 | 1347.702 | 0 | −0.3 | 2464 | 454.7 | 454.7 | 454.7 | 6.90 |
| 1926 | R.VREAENVAFANK.D | | 449.906 | 3 | 1347.702 | 1347.702 | 0 | 0.2 | 2464 | 359.2 | 359.2 | 359.2 | 5.82 |
| 890 | R.LESAGLGYR.V | | 483.256 | 2 | 965.505 | 965.505 | 0 | −0.1 | 2538 | 570.3 | 570.3 | 570.3 | 8.83 |
| 890 | R.LESAGLGYR.V | | 483.256 | 2 | 965.505 | 965.505 | 0 | 0.1 | 2538 | 547.4 | 547.4 | 547.4 | 8.10 |
| 890 | R.LESAGLGYR.V | | 483.256 | 2 | 965.505 | 965.505 | 0 | 0.3 | 2538 | 523.9 | 523.9 | 523.9 | 7.51 |
| 890 | R.LESAGLGYR.V | | 483.256 | 2 | 965.505 | 965.505 | 0 | 0.2 | 2538 | 327.2 | 327.2 | 327.2 | 5.91 |
| 1190 | R.VSMEETADRLGSIPLR.Q | | 591.977 | 3 | 1773.916 | 1773.916 | 0 | 0.0 | 2547 | 650.6 | 650.6 | 650.6 | 11.44 |
| 1190 | R.VSMEETADRLGSIPLR.Q | | 591.977 | 3 | 1773.917 | 1773.916 | 0 | 0.3 | 2547 | 582.1 | 582.1 | 582.1 | 9.44 |
| 1587 | R.VSM[+15.995]EETADRLGSIPLR.Q | M[+16] | 597.309 | 3 | 1789.911 | 1789.911 | 0 | 0.0 | 2547 | 502.2 | 465.8 | 465.8 | 7.94 |
| 1587 | R.VSM[+15.995]EETADRLGSIPLR.Q | M[+16] | 597.308 | 3 | 1789.911 | 1789.911 | 0 | −0.4 | 2547 | 469.1 | 469.1 | 469.1 | 7.19 |
| 1190 | R.VSMEETADRLGSIPLR.Q | | 591.977 | 3 | 1773.916 | 1773.916 | 0 | −0.1 | 2547 | 377.7 | 377.7 | 377.7 | 7.14 |
| 1587 | R.VSM[+15.995]EETADRLGSIPLR.Q | M[+16] | 597.309 | 3 | 1789.911 | 1789.911 | 0 | −0.2 | 2547 | 406.1 | 406.1 | 406.1 | 6.68 |
| 1190 | R.VSMEETADRLGSIPLR.Q | | 591.977 | 3 | 1773.917 | 1773.916 | 0 | 0.1 | 2547 | 332.7 | 332.7 | 332.7 | 5.63 |
| 1547 | R.VSM[+15.995JEETADR.L | M[+16] | 527.230 | 2 | 1053.452 | 1053.452 | 0 | 0.1 | 2547 | 333.9 | 333.9 | 333.9 | 5.42 |
| 2512 | R.LGSIPLRQLVYR.V | | 472.289 | 3 | 1414.853 | 1414.853 | 0 | −0.3 | 2556 | 524.3 | 524.3 | 524.3 | 7.53 |
| 1234 | R.VHALPPSLIPLVWDFGQLSDVAEKLYIQQIVQR.L | | 944.026 | 4 | 3773.082 | 3773.079 | 0 | 0.8 | 2568 | 637.0 | 637.0 | 637.0 | 9.99 |
| 1139 | R.VHALPPSLIPLVWDFGQLSDVAEK.L | | 877.810 | 3 | 2631.416 | 2631.418 | 0 | −0.6 | 2568 | 322.5 | 322.5 | 322.5 | 5.58 |
| 981 | K.LYIQQIVQR.L | | 580.843 | 2 | 1160.678 | 1160.679 | 0 | 0.2 | 2592 | 586.5 | 586.5 | 586.5 | 8.54 |
| 981 | K.LYIQQIVQR.L | | 580.843 | 2 | 1160.679 | 1160.679 | 0 | 0.1 | 2592 | 303.6 | 303.6 | 303.6 | 5.43 |
| 2006 | R.LVESISLDENGTR.V | | 716.867 | 2 | 1432.728 | 1432.728 | 0 | −0.2 | 2601 | 437.0 | 422.5 | 422.5 | 8.42 |
| 2006 | R.LVESISLDENGTR.V | | 716.868 | 2 | 1432.728 | 1432.728 | 0 | 0.3 | 2601 | 359.5 | 316.9 | 316.9 | 5.89 |
| 2006 | R.LVESISLDENGTR.V | | 478.582 | 3 | 1433.730 | 1432.728 | 1 | −0.6 | 2601 | 460.2 | 445.1 | 445.1 | 5.27 |
| 883 | R.FFPKPYDDSRLLLDEITR.A | | 742.392 | 3 | 2225.161 | 2225.160 | 0 | 0.3 | 2710 | 409.4 | 409.4 | 409.4 | 7.81 |
| 883 | R.FFPKPYDDSRLLLDEITR.A | | 557.046 | 4 | 2225.160 | 2225.160 | 0 | 0.0 | 2710 | 396.6 | 396.6 | 396.6 | 7.70 |
| 883 | R.FFPKPYDDSRLLLDEITR.A | | 557.046 | 4 | 2225.162 | 2225.160 | 0 | 0.7 | 2710 | 378.6 | 378.6 | 378.6 | 7.70 |
| 938 | R.FFPKPYDDSR.L | | 636.307 | 2 | 1271.606 | 1271.606 | 0 | 0.3 | 2710 | 460.2 | 460.2 | 460.2 | 7.57 |
| 938 | R.FFPKPYDDSR.L | | 424.540 | 3 | 1271.606 | 1271.606 | 0 | 0.1 | 2710 | 401.6 | 401.6 | 401.6 | 7.35 |
| 938 | R.FFPKPYDDSR.L | | 636.306 | 2 | 1271.605 | 1271.606 | 0 | −0.3 | 2710 | 519.6 | 519.6 | 519.6 | 7.33 |
| 938 | R.FFPKPYDDSR.L | | 424.540 | 3 | 1271.606 | 1271.606 | 0 | 0.3 | 2710 | 368.3 | 368.3 | 368.3 | 7.23 |
| 883 | R.FFPKPYDDSRLLLDEITR.A | | 1113.084 | 2 | 2225.160 | 2225.160 | 0 | 0.1 | 2710 | 445.4 | 445.4 | 445.4 | 7.22 |
| 938 | R.FFPKPYDDSR.L | | 424.540 | 3 | 1271.606 | 1271.606 | 0 | 0.2 | 2710 | 362.1 | 362.1 | 362.1 | 7.03 |
| 938 | R.FFPKPYDDSR.L | | 424.540 | 3 | 1271.606 | 1271.606 | 0 | 0.4 | 2710 | 356.2 | 356.2 | 356.2 | 5.91 |
| 938 | R.FFPKPYDDSR.L | | 424.540 | 3 | 1271.606 | 1271.606 | 0 | 0.2 | 2710 | 335.7 | 335.7 | 335.7 | 5.91 |
| 938 | R.FFPKPYDDSR.L | | 424.540 | 3 | 1271.606 | 1271.606 | 0 | 0.0 | 2710 | 341.9 | 341.9 | 341.9 | 5.71 |
| 938 | R.FFPKPYDDSR.L | | 636.306 | 2 | 1271.605 | 1271.606 | 0 | 0.1 | 2710 | 327.0 | 327.0 | 327.0 | 5.71 |
| 902 | R.LLLDEITRAQDLFLDGVPLRK.T | | 809.132 | 3 | 2425.382 | 2425.381 | 0 | 0.1 | 2720 | 699.1 | 699.1 | 699.1 | 11.36 |
| 1256 | R.LLLDEITRAQDLFLDGVPLR.K | | 766.434 | 3 | 2297.287 | 2297.286 | 0 | 0.1 | 2720 | 581.5 | 581.5 | 581.5 | 9.70 |
| 902 | R.LLLDEITRAQDLFLDGVPLRK.T | | 809.132 | 3 | 2425.382 | 2425.381 | 0 | 0.3 | 2720 | 594.2 | 594.2 | 594.2 | 9.36 |
| 1256 | R.LLLDEITRAQDLFLDGVPLR.K | | 766.434 | 3 | 2297.286 | 2297.286 | 0 | −0.1 | 2720 | 553.0 | 553.0 | 553.0 | 8.97 |
| 902 | R.LLLDEITRAQDLFLDGVPLRK.T | | 607.101 | 4 | 2425.380 | 2425.381 | 0 | −0.4 | 2720 | 429.2 | 429.2 | 429.2 | 7.48 |
| 2028 | R.LLLDEITR.A | | 486.790 | 2 | 972.573 | 972.572 | 0 | 0.2 | 2720 | 465.6 | 465.6 | 465.6 | 7.38 |
| 2028 | R.LLLDEITR.A | | 486.790 | 2 | 972.573 | 972.572 | 0 | 0.4 | 2720 | 438.3 | 438.3 | 438.3 | 7.35 |
| 2028 | R.LLLDEITR.A | | 972.573 | 1 | 972.573 | 972.572 | 0 | 0.1 | 2720 | 424.4 | 424.4 | 424.4 | 7.35 |
| 902 | R.LLLDEITRAQDLFLDGVPLRK.T | | 607.101 | 4 | 2425.382 | 2425.381 | 0 | 0.4 | 2720 | 376.9 | 376.9 | 376.9 | 7.35 |
| 2028 | R.LLLDEITR.A | | 486.790 | 2 | 972.572 | 972.572 | 0 | −0.2 | 2720 | 383.7 | 383.7 | 383.7 | 7.03 |
| 902 | R.LLLDEITRAQDLFLDGVPLRK.T | | 485.882 | 5 | 2425.381 | 2425.381 | 0 | −0.1 | 2720 | 379.4 | 379.4 | 379.4 | 6.83 |
| 902 | R.LLLDEITRAQDLFLDGVPLRK.T | | 607.101 | 4 | 2425.383 | 2425.381 | 0 | 0.6 | 2720 | 335.7 | 335.7 | 335.7 | 5.55 |
| 922 | R.AQDLFLDGVPLRK.T | | 491.280 | 3 | 1471.827 | 1471.827 | 0 | 0.0 | 2728 | 666.3 | 666.3 | 666.3 | 11.44 |
| 922 | R.AQDLFLDGVPLRK.T | | 491.280 | 3 | 1471.827 | 1471.827 | 0 | 0.0 | 2728 | 637.7 | 637.7 | 637.7 | 10.44 |
| 922 | R.AQDLFLDGVPLRK.T | | 736.417 | 2 | 1471.827 | 1471.827 | 0 | −0.2 | 2728 | 599.0 | 599.0 | 599.0 | 8.86 |
| 922 | R.AQDLFLDGVPLRK.T | | 491.280 | 3 | 1471.827 | 1471.827 | 0 | 0.0 | 2728 | 503.5 | 503.5 | 503.5 | 8.53 |
| 922 | R.AQDLFLDGVPLRK.T | | 491.280 | 3 | 1471.827 | 1471.827 | 0 | 0.0 | 2728 | 487.2 | 487.2 | 487.2 | 8.53 |
| 922 | R.AQDLFLDGVPLRK.T | | 736.417 | 2 | 1471.827 | 1471.827 | 0 | −0.2 | 2728 | 557.1 | 557.1 | 557.1 | 8.13 |
| 922 | R.AQDLFLDGVPLRK.T | | 736.417 | 2 | 1471.827 | 1471.827 | 0 | 0.0 | 2728 | 552.9 | 552.9 | 552.9 | 8.12 |
| 922 | R.AQDLFLDGVPLRK.T | | 491.280 | 3 | 1471.827 | 1471.827 | 0 | −0.1 | 2728 | 457.9 | 457.9 | 457.9 | 7.78 |
| 922 | R.AQDLFLDGVPLRK.T | | 491.281 | 3 | 1471.827 | 1471.827 | 0 | 0.1 | 2728 | 448.4 | 448.4 | 448.4 | 7.78 |
| 922 | R.AQDLFLDGVPLRK.T | | 736.417 | 2 | 1471.827 | 1471.827 | 0 | 0.0 | 2728 | 455.3 | 455.3 | 455.3 | 7.49 |
| 922 | R.AQDLFLDGVPLRK.T | | 491.280 | 3 | 1471.826 | 1471.827 | 0 | −0.3 | 2728 | 501.9 | 501.9 | 501.9 | 7.35 |
| 922 | R.AQDLFLDGVPLRK.T | | 736.417 | 2 | 1471.827 | 1471.827 | 0 | −0.1 | 2728 | 402.2 | 402.2 | 402.2 | 7.26 |
| 922 | R.AQDLFLDGVPLRK.T | | 736.417 | 2 | 1471.827 | 1471.827 | 0 | −0.2 | 2728 | 467.1 | 467.1 | 467.1 | 7.19 |
| 922 | R.AQDLFLDGVPLRK.T | | 491.281 | 3 | 1471.827 | 1471.827 | 0 | 0.1 | 2728 | 353.5 | 353.5 | 353.5 | 5.82 |
| 2639 | K.IPLFLVGKPGSSK.S | | 448.275 | 3 | 1342.809 | 1342.809 | 0 | 0.1 | 2763 | 557.5 | 557.5 | 557.5 | 8.98 |
| 1545 | K.TIVADAM[+15.995]QGPAAYSDLFR.S | M[+16] | 971.472 | 2 | 1941.937 | 1941.938 | 0 | 0.2 | 2780 | 660.3 | 660.3 | 660.3 | 11.98 |
| 850 | K.TIVADAMQGPAAYSDLFR.S | | 963.475 | 2 | 1925.943 | 1925.943 | 0 | 0.0 | 2780 | 640.1 | 640.1 | 640.1 | 10.98 |
| 850 | K.TIVADAMQGPAAYSDLFR.S | | 642.653 | 3 | 1925.944 | 1925.943 | 0 | 0.7 | 2780 | 640.4 | 640.4 | 640.4 | 10.46 |
| 850 | K.TIVADAMQGPAAYSDLFR.S | | 963.475 | 2 | 1925.942 | 1925.943 | 0 | −0.3 | 2780 | 604.4 | 604.4 | 604.4 | 10.39 |
| 850 | K.TIVADAMQGPAAYSDLFR.S | | 963.475 | 2 | 1925.943 | 1925.943 | 0 | 0.2 | 2780 | 591.8 | 591.8 | 591.8 | 9.98 |
| 850 | K.TIVADAMQGPAAYSDLFR.S | | 963.476 | 2 | 1925.945 | 1925.943 | 0 | 1.1 | 2780 | 589.9 | 589.9 | 589.9 | 9.98 |
| 850 | K.TIVADAMQGPAAYSDLFR.S | | 642.653 | 3 | 1925.943 | 1925.943 | 0 | 0.2 | 2780 | 594.1 | 594.1 | 594.1 | 9.46 |
| 850 | K.TIVADAMQGPAAYSDLFR.S | | 642.653 | 3 | 1925.946 | 1925.943 | 0 | 1.5 | 2780 | 579.5 | 579.5 | 579.5 | 9.46 |
| 850 | K.TIVADAMQGPAAYSDLFR.S | | 963.475 | 2 | 1925.942 | 1925.943 | 0 | −0.2 | 2780 | 594.8 | 594.8 | 594.8 | 9.39 |
| 850 | K.TIVADAMQGPAAYSDLFR.S | | 963.475 | 2 | 1925.944 | 1925.943 | 0 | 0.5 | 2780 | 447.3 | 447.3 | 447.3 | 8.91 |
| 850 | K.TIVADAMQGPAAYSDLFR.S | | 642.653 | 3 | 1925.943 | 1925.943 | 0 | 0.2 | 2780 | 549.2 | 549.2 | 549.2 | 8.73 |
| 850 | K.TIVADAMQGPAAYSDLFR.S | | 642.653 | 3 | 1925.944 | 1925.943 | 0 | 0.6 | 2780 | 518.5 | 518.5 | 518.5 | 8.55 |
| 850 | K.TIVADAMQGPAAYSDLFR.S | | 642.653 | 3 | 1925.944 | 1925.943 | 0 | 0.7 | 2780 | 509.9 | 509.9 | 509.9 | 8.55 |
| 850 | K.TIVADAMQGPAAYSDLFR.S | | 963.475 | 2 | 1925.942 | 1925.943 | 0 | −0.2 | 2780 | 426.8 | 426.8 | 426.8 | 8.10 |
| 850 | K.TIVADAMQGPAAYSDLFR.S | | 963.475 | 2 | 1925.943 | 1925.943 | 0 | 0.1 | 2780 | 383.3 | 383.3 | 383.3 | 7.97 |
| 850 | K.TIVADAMQGPAAYSDLFR.S | | 642.653 | 3 | 1925.943 | 1925.943 | 0 | 0.4 | 2780 | 391.7 | 391.7 | 391.7 | 7.45 |
| 850 | K.TIVADAMQGPAAYSDLFR.S | | 642.653 | 3 | 1925.943 | 1925.943 | 0 | 0.2 | 2780 | 336.4 | 336.4 | 336.4 | 5.65 |
| 1688 | R.SLKQVHLVSFQC[+57.021]SPHSTPQGIISTFR.Q | C[+57] | 739.388 | 4 | 2954.531 | 2954.531 | 0 | 0.2 | 2798 | 749.0 | 749.0 | 749.0 | 12.70 |
| 1700 | K.QVHLVSFQC[+57.021]SPHSTPQGIISTFR.Q | C[+57] | 657.586 | 4 | 2627.321 | 2626.320 | 1 | −0.8 | 2801 | 325.8 | 325.8 | 325.8 | 5.58 |
| 1416 | R.FQQGKDLQQYVSVVVLDEVGLAEDSPKMPLK.T | | 1154.272 | 3 | 3460.803 | 3460.803 | 0 | 0.2 | 2828 | 783.3 | 783.3 | 783.3 | 12.64 |
| 1416 | R.FQQGKDLQQYVSVVVLDEVGLAEDSPKMPLK.T | | 865.957 | 4 | 3460.804 | 3460.803 | 0 | 0.2 | 2828 | 751.1 | 751.1 | 751.1 | 12.64 |
| 961 | R.FQQGKDLQQYVSVVVLDEVGLAEDSPK.M | | 1496.271 | 2 | 2991.535 | 2991.531 | 0 | 1.2 | 2828 | 602.0 | 602.0 | 602.0 | 10.70 |
| 1416 | R.FQQGKDLQQYVSVVVLDEVGLAEDSPKMPLK.T | | 865.956 | 4 | 3460.803 | 3460.803 | 0 | −0.1 | 2828 | 695.1 | 695.1 | 695.1 | 10.64 |
| 1416 | R.FQQGKDLQQYVSVVVLDEVGLAEDSPKMPLK.T | | 865.956 | 4 | 3460.803 | 3460.803 | 0 | 0.0 | 2828 | 653.6 | 653.6 | 653.6 | 10.64 |
| 1416 | R.FQQGKDLQQYVSVVVLDEVGLAEDSPKMPLK.T | | 865.956 | 4 | 3460.802 | 3460.803 | 0 | 0.5 | 2828 | 654.0 | 654.0 | 654.0 | 10.06 |
| 961 | R.FQQGKDLQQYVSVVVLDEVGLAEDSPK.M | | 1496.270 | 2 | 2991.532 | 2991.531 | 0 | 0.4 | 2828 | 533.7 | 533.7 | 533.7 | 8.97 |
| 1416 | R.FQQGKDLQQYVSVVVLDEVGLAEDSPKMPLK.T | | 692.966 | 5 | 3460.803 | 3460.803 | 0 | −0.2 | 2828 | 639.2 | 639.2 | 639.2 | 8.54 |
| 1416 | R.FQQGKDLQQYVSVVVLDEVGLAEDSPKMPLK.T | | 865.957 | 4 | 3460.804 | 3460.803 | 0 | 0.2 | 2828 | 555.5 | 555.5 | 555.5 | 7.91 |
| 961 | R.FQQGKDLQQYVSVVVLDEVGLAEDSPK.M | | 997.849 | 3 | 2991.533 | 2991.531 | 0 | 0.8 | 2828 | 450.0 | 450.0 | 450.0 | 7.75 |
| 961 | R.FQQGKDLQQYVSVVVLDEVGLAEDSPK.M | | 748.639 | 4 | 2991.533 | 2991.531 | 0 | 0.8 | 2828 | 462.2 | 462.2 | 462.2 | 7.52 |
| 1416 | R.FQQGKDLQQYVSVVVLDEVGLAEDSPKMPLK.T | | 692.967 | 5 | 3460.804 | 3460.803 | 0 | 0.2 | 2828 | 530.5 | 530.5 | 530.5 | 7.39 |
| 961 | R.FQQGKDLQQYVSVVVLDEVGLAEDSPK.M | | 997.850 | 3 | 2991.534 | 2991.531 | 0 | 1.0 | 2828 | 395.6 | 395.6 | 395.6 | 7.22 |
| 1617 | R.FQQGKDLQQYVSVVVLDEVGLAEDSPKM[+15.995]PLK.T | M[+16] | 869.955 | 4 | 3476.799 | 3476.798 | 0 | 0.4 | 2828 | 488.0 | 488.0 | 488.0 | 7.14 |
| 961 | R.FQQGKDLQQYVSVVVLDEVGLAEDSPK.M | | 748.639 | 4 | 2991.533 | 2991.531 | 0 | 0.5 | 2828 | 400.2 | 400.2 | 400.2 | 7.00 |
| 1617 | R.FQQGKDLQQYVSVVVLDEVGLAEDSPKM[+15.995]PLK.T | M[+16] | 696.166 | 5 | 3476.801 | 3476.798 | 0 | 0.8 | 2828 | 471.4 | 471.4 | 471.4 | 6.17 |
| 961 | R.FQQGKDLQQYVSVVVLDEVGLAEDSPK.M | | 748.639 | 4 | 2991.533 | 2991.531 | 0 | 0.5 | 2828 | 336.5 | 336.5 | 336.5 | 5.56 |
| 961 | R.FQQGKDLQQYVSVVVLDEVGLAEDSPK.M | | 748.639 | 4 | 2991.533 | 2991.531 | 0 | 0.7 | 2828 | 358.9 | 358.9 | 358.9 | 5.18 |
| 961 | R.FQQGKDLQQYVSVVVLDEVGLAEDSPK.M | | 748.639 | 4 | 2991.533 | 2991.531 | 0 | 0.5 | 2828 | 309.4 | 309.4 | 309.4 | 5.09 |
| 1546 | K.DLQQYVSVVVLDEVGLAEDSPKM[+15.995]PLK.T | M[+16] | 722.880 | 4 | 2888.497 | 2888.496 | 0 | 0.3 | 2833 | 629.1 | 629.1 | 629.1 | 10.19 |
| 932 | K.DLQQYVSVVVLDEVGLAEDSPK.M | | 1202.120 | 2 | 2403.232 | 2403.229 | 0 | 1.4 | 2833 | 530.8 | 449.4 | 449.4 | 9.25 |
| 1171 | K.DLQQYVSVVVLDEVGLAEDSPKMPLK.T | | 958.173 | 3 | 2872.504 | 2872.501 | 0 | 0.9 | 2833 | 571.1 | 536.8 | 536.8 | 9.11 |
| 1171 | K.DLQQYVSVVVLDEVGLAEDSPKMPLK.T | | 958.174 | 3 | 2872.507 | 2872.501 | 0 | 1.9 | 2833 | 554.1 | 554.1 | 554.1 | 8.97 |
| 1171 | K.DLQQYVSVVVLDEVGLAEDSPKMPLK.T | | 1436.756 | 2 | 2872.506 | 2872.501 | 0 | 1.5 | 2833 | 473.8 | 389.5 | 389.5 | 8.64 |
| 932 | K.DLQQYVSVVVLDEVGLAEDSPK.M | | 801.749 | 3 | 2403.232 | 2403.229 | 0 | 1.2 | 2833 | 487.8 | 427.5 | 427.5 | 7.95 |
| 1546 | K.DLQQYVSVVVLDEVGLAEDSPKM[+15.995]PLK.T | M[+16] | 722.880 | 4 | 2888.496 | 2888.496 | 0 | 0.0 | 2833 | 558.9 | 551.5 | 551.5 | 7.86 |
| 1171 | K.DLQQYVSVVVLDEVGLAEDSPKMPLK.T | | 575.306 | 5 | 2872.502 | 2872.501 | 0 | 0.2 | 2833 | 472.4 | 472.4 | 472.4 | 7.52 |
| 1686 | K.MPLKTLHPLLEDGC[+57.021]IEDDPAPHKK.V | C[+57] | 689.355 | 4 | 2754.397 | 2754.395 | 0 | 0.7 | 2855 | 581.4 | 581.4 | 581.4 | 7.94 |
| 1686 | K.MPLKTLHPLLEDGC[+57.021]IEDDPAPHKK.V | C[+57] | 459.905 | 6 | 2754.395 | 2754.395 | 0 | 0.0 | 2855 | 414.2 | 414.2 | 414.2 | 7.47 |
| 1723 | K.TLHPLLEDGC[+57.021]IEDDPAPHKK.V | C[+57] | 572.036 | 4 | 2285.121 | 2285.123 | 0 | 0.9 | 2859 | 867.9 | 867.9 | 867.9 | 15.11 |
| 1723 | K.TLHPLLEDGC[+57.021]IEDDPAPHKK.V | C[+57] | 457.830 | 5 | 2285.123 | 2285.123 | 0 | 0.0 | 2859 | 723.2 | 670.0 | 670.0 | 12.70 |
| 1723 | K.TLHPLLEDGC[+57.021]IEDDPAPHKK.V | C[+57] | 762.380 | 3 | 2285.124 | 2285.123 | 0 | 0.4 | 2859 | 703.1 | 703.1 | 703.1 | 11.28 |
| 1723 | K.TLHPLLEDGC[+57.021]IEDDPAPHKK.V | C[+57] | 572.036 | 4 | 2285.123 | 2285.123 | 0 | 0.1 | 2859 | 542.4 | 542.4 | 542.4 | 8.97 |
| 1966 | K.KVGFVGISNWALDPAK.M | | 567.982 | 3 | 1701.932 | 1701.932 | 0 | 0.0 | 2878 | 351.5 | 351.5 | 351.5 | 5.91 |
| 836 | K.VGFVGISNWALDPAK.M | | 787.422 | 2 | 1573.837 | 1573.837 | 0 | −0.1 | 2879 | 662.9 | 662.9 | 662.9 | 11.72 |
| 1530 | K.VGFVGISNWALDPAKM[+15.995]NR.G | M[+16] | 664.345 | 3 | 1991.019 | 1991.017 | 0 | 1.2 | 2879 | 660.5 | 660.5 | 660.5 | 11.70 |
| 1530 | K.VGFVGISNWALDPAKM[+15.995]NR.G | M[+16] | 664.344 | 3 | 1991.018 | 1991.017 | 0 | 0.8 | 2879 | 628.6 | 628.6 | 628.6 | 10.70 |
| 1006 | K.VGFVGISNWALDPAKMNR.G | | 659.012 | 3 | 1975.023 | 1975.022 | 0 | 0.4 | 2879 | 582.1 | 582.1 | 582.1 | 9.70 |
| 836 | K.VGFVGISNWALDPAK.M | | 787.422 | 2 | 1573.836 | 1573.837 | 0 | −0.6 | 2879 | 577.6 | 577.6 | 577.6 | 9.13 |
| 836 | K.VGFVGISNWALDPAK.M | | 787.422 | 2 | 1573.836 | 1573.837 | 0 | −0.6 | 2879 | 566.1 | 566.1 | 566.1 | 9.13 |
| 1530 | K.VGFVGISNWALDPAKM[+15.995]NR.G | M[+16] | 996.012 | 2 | 1991.017 | 1991.017 | 0 | 0.1 | 2879 | 478.9 | 478.9 | 478.9 | 8.64 |
| 1006 | K.VGFVGISNWALDPAKMNR.G | | 659.012 | 3 | 1975.020 | 1975.022 | 0 | 0.8 | 2879 | 557.6 | 557.6 | 557.6 | 8.39 |
| 836 | K.VGFVGISNWALDPAK.M | | 787.422 | 2 | 1573.837 | 1573.837 | 0 | −0.4 | 2879 | 501.2 | 501.2 | 501.2 | 8.22 |
| 836 | K.VGFVGISNWALDPAK.M | | 787.422 | 2 | 1573.838 | 1573.837 | 0 | 0.1 | 2879 | 510.7 | 510.7 | 510.7 | 8.21 |
| 836 | K.VGFVGISNWALDPAK.M | | 525.284 | 3 | 1573.837 | 1573.837 | 0 | −0.3 | 2879 | 454.8 | 454.8 | 454.8 | 6.66 |
| 1530 | K.VGFVGISNWALDPAKM[+15.995]NR.G | M[+16] | 664.344 | 3 | 1991.018 | 1991.017 | 0 | 0.4 | 2879 | 319.8 | 319.8 | 319.8 | 5.80 |
| 836 | K.VGFVGISNWALDPAK.M | | 525.618 | 3 | 1574.840 | 1573.837 | 1 | −0.2 | 2879 | 389.3 | 389.3 | 389.3 | 5.22 |
| 1105 | R.GIFVSRGSPNETELIESAK.G | | 678.689 | 3 | 2034.051 | 2034.050 | 0 | 0.5 | 2897 | 508.2 | 498.7 | 498.7 | 8.79 |
| 990 | R.GSPNETELIESAK.G | | 687.841 | 2 | 1374.675 | 1374.675 | 0 | 0.2 | 2903 | 640.9 | 640.9 | 640.9 | 10.72 |
| 990 | R.GSPNETELIESAK.G | | 687.841 | 2 | 1374.675 | 1374.675 | 0 | 0.0 | 2903 | 632.4 | 632.4 | 632.4 | 10.72 |
| 1737 | R.GSPNETELIESAKGIC[+57.021]SSDILVQDR.V | C[+57] | 906.780 | 3 | 2718.326 | 2718.325 | 0 | 0.5 | 2903 | 603.2 | 603.2 | 603.2 | 10.11 |
| 990 | R.GSPNETELIESAK.G | | 687.841 | 2 | 1374.675 | 1374.675 | 0 | −0.1 | 2903 | 590.4 | 590.4 | 590.4 | 9.72 |
| 990 | R.GSPNETELIESAK.G | | 458.896 | 3 | 1374.675 | 1374.675 | 0 | −0.1 | 2903 | 560.3 | 559.2 | 559.2 | 9.20 |
| 990 | R.GSPNETELIESAK.G | | 458.896 | 3 | 1374.675 | 1374.675 | 0 | −0.1 | 2903 | 464.9 | 450.9 | 450.9 | 7.53 |
| 990 | R.GSPNETELIESAK.G | | 458.896 | 3 | 1374.675 | 1374.675 | 0 | −0.1 | 2903 | 347.6 | 347.6 | 347.6 | 5.58 |
| 990 | R.GSPNETELIESAK.G | | 687.840 | 2 | 1374.673 | 1374.675 | 0 | −1.1 | 2903 | 346.5 | 346.5 | 346.5 | 5.51 |
| 990 | R.GSPNETELIESAK.G | | 458.896 | 3 | 1374.675 | 1374.675 | 0 | −0.1 | 2903 | 341.7 | 341.7 | 341.7 | 5.19 |
| 1862 | R.VQGYFASFAK.A | | 559.287 | 2 | 1117.567 | 1117.568 | 0 | −0.3 | 2928 | 436.1 | 436.1 | 436.1 | 6.95 |
| 1862 | R.VQGYFASFAK.A | | 559.287 | 2 | 1117.567 | 1117.568 | 0 | −0.4 | 2928 | 319.3 | 319.3 | 319.3 | 4.85 |
| 877 | K.RQDKEFFGLR.D | | 432.567 | 3 | 1295.686 | 1295.686 | 0 | 0.3 | 2945 | 503.5 | 503.5 | 503.5 | 7.64 |
| 2123 | R.QDKEFFGLRDYYSLIK.M | | 506.264 | 4 | 2022.033 | 2022.033 | 0 | 0.0 | 2946 | 395.2 | 395.2 | 395.2 | 7.09 |
| 2041 | R.DYYSLIK.M | | 451.237 | 2 | 901.467 | 901.467 | 0 | 0.4 | 2955 | 396.1 | 396.1 | 396.1 | 6.74 |
| 1007 | K.ASNRKPSPQDIAQAVLR.N | | 617.678 | 3 | 1851.020 | 1851.020 | 0 | 0.2 | 2969 | 644.3 | 644.3 | 644.3 | 10.70 |
| 1007 | K.ASNRKPSPQDIAQAVLR.N | | 617.678 | 3 | 1851.020 | 1851.020 | 0 | 0.1 | 2969 | 446.1 | 446.1 | 446.1 | 8.64 |
| 1007 | K.ASNRKPSPQDIAQAVLR.N | | 617.678 | 3 | 1851.020 | 1851.020 | 0 | 0.4 | 2969 | 464.2 | 464.2 | 464.2 | 8.04 |
| 1007 | K.ASNRKPSPQDIAQAVLR.N | | 617.678 | 3 | 1851.020 | 1851.020 | 0 | 0.1 | 2969 | 389.8 | 389.8 | 389.8 | 7.70 |
| 839 | R.KPSPQDIAQAVLR.N | | 474.940 | 3 | 1422.806 | 1422.806 | 0 | −0.3 | 2973 | 532.2 | 532.2 | 532.2 | 8.40 |
| 839 | R.KPSPQDIAQAVLR.N | | 474.940 | 3 | 1422.806 | 1422.806 | 0 | 0.0 | 2973 | 440.3 | 440.3 | 440.3 | 7.76 |
| 839 | R.KPSPQDIAQAVLR.N | | 474.940 | 3 | 1422.806 | 1422.806 | 0 | −0.2 | 2973 | 396.0 | 396.0 | 396.0 | 7.42 |
| 839 | R.KPSPQDIAQAVLR.N | | 474.940 | 3 | 1422.806 | 1422.806 | 0 | 0.0 | 2973 | 371.4 | 371.4 | 371.4 | 7.23 |
| 839 | R.KPSPQDIAQAVLR.N | | 475.275 | 3 | 1423.809 | 1422.806 | 1 | −0.3 | 2973 | 388.9 | 305.9 | 305.9 | 3.72 |
| 815 | R.NFSGKDDIQALDIFLANLPEAK.C | | 1210.129 | 2 | 2419.250 | 2419.250 | 0 | −0.1 | 2986 | 607.9 | 607.9 | 607.9 | 10.70 |
| 815 | R.NFSGKDDIQALDIFLANLPEAK.C | | 1210.129 | 2 | 2419.252 | 2419.250 | 0 | 0.5 | 2986 | 568.7 | 523.7 | 523.7 | 9.70 |
| 1490 | R.N[+0.984]FSGKDDIQALDIFLANLPEAK.C | N[+1] | 807.417 | 3 | 2420.235 | 2420.234 | 0 | 0.4 | 2986 | 412.8 | 412.8 | 339.0 | 7.81 |
| 815 | R.NFSGKDDIQALDIFLANLPEAK.C | | 807.088 | 3 | 2419.250 | 2419.250 | 0 | 0.0 | 2986 | 395.6 | 395.6 | 395.6 | 7.70 |
| 815 | R.NFSGKDDIQALDIFLANLPEAK.C | | 807.089 | 3 | 2419.252 | 2419.250 | 0 | 0.5 | 2986 | 444.1 | 444.1 | 444.1 | 7.56 |
| 1490 | R.N[+0.984]FSGKDDIQALDIFLANLPEAK.C | N[+1] | 807.416 | 3 | 2420.235 | 2420.234 | 0 | 0.1 | 2986 | 410.3 | 410.3 | 319.4 | 7.52 |
| 815 | R.NFSGKDDIQALDIFLANLPEAK.C | | 807.089 | 3 | 2419.254 | 2419.250 | 0 | 1.4 | 2986 | 433.4 | 433.4 | 433.4 | 7.33 |
| 815 | R.NFSGKDDIQALDIFLANLPEAK.C | | 807.089 | 3 | 2419.251 | 2419.250 | 0 | 0.3 | 2986 | 364.5 | 364.5 | 364.5 | 7.22 |
| 815 | R.NFSGKDDIQALDIFLANLPEAK.C | | 605.568 | 4 | 2419.252 | 2419.250 | 0 | 0.5 | 2986 | 321.2 | 321.2 | 321.2 | 5.56 |
| 815 | R.NFSGKDDIQALDIFLANLPEAK.C | | 807.089 | 3 | 2419.251 | 2419.250 | 0 | 0.4 | 2986 | 317.2 | 295.8 | 295.8 | 5.12 |
| 815 | R.NFSGKDDIQALDIFLANLPEAK.C | | 807.090 | 3 | 2419.254 | 2419.250 | 0 | 1.6 | 2986 | 306.7 | 306.7 | 306.7 | 5.12 |
| 876 | K.DDIQALDIFLANLPEAK.C | | 629.336 | 3 | 1885.992 | 1885.991 | 0 | 0.9 | 2991 | 474.6 | 474.6 | 474.6 | 7.79 |
| 1717 | K.C[+57.021]SEEVSPMQLIK.Q | C[+57 | 710.844 | 2 | 1420.681 | 1420.681 | 0 | 0.3 | 3008 | 509.3 | 422.2 | 422.2 | 8.80 |
| 1717 | K.C[+57.021]SEEVSPMQLIK.Q | C[+57] | 710.844 | 2 | 1420.681 | 1420.681 | 0 | −0.2 | 3008 | 500.8 | 463.9 | 463.9 | 8.80 |
| 1671 | K.C[+57.021]SEEVSPM[+15.995]QLIK.Q | C[+57], | 718.842 | 2 | 1436.676 | 1436.676 | 0 | 0.1 | 3008 | 467.0 | 299.7 | 299.7 | 6.61 |
| | | M[+16] | | | | | | | | | | | |
| 1166 | K.QNIFGPSQKVPGGEQEDAESR.Y | | 758.365 | 3 | 2273.079 | 2273.079 | 0 | −0.1 | 3020 | 873.7 | 873.7 | 873.7 | 15.65 |
| 1166 | K.QNIFGPSQKVPGGEQEDAESR.Y | | 758.365 | 3 | 2273.079 | 2273.079 | 0 | −0.1 | 3020 | 843.4 | 843.4 | 843.4 | 14.70 |
| 1166 | K.QNIFGPSQKVPGGEQEDAESR.Y | | 758.365 | 3 | 2273.080 | 2273.079 | 0 | 0.2 | 3020 | 745.6 | 745.6 | 745.6 | 12.70 |
| 940 | K.QNIFGPSQKVPGGEQEDAESRYLLVLTK.N | | 1035.207 | 3 | 3103.605 | 3103.606 | 0 | −0.2 | 3020 | 681.6 | 681.6 | 681.6 | 10.77 |
| 940 | K.QNIFGPSQKVPGGEQEDAESRYLLVLTK.N | | 776.657 | 4 | 3103.607 | 3103.606 | 0 | 0.3 | 3020 | 625.3 | 625.3 | 625.3 | 10.36 |
| 940 | K.QNIFGPSQKVPGGEQEDAESRYLLVLTK.N | | 776.657 | 4 | 3103.605 | 3103.606 | 0 | −0.3 | 3020 | 504.3 | 504.3 | 504.3 | 7.86 |
| 1784 | K.VPGGEQEDAESRYLLVLTK.N | | 702.036 | 3 | 2104.092 | 2104.092 | 0 | 0.0 | 3029 | 303.6 | 303.6 | 303.6 | 5.42 |
| 1950 | R.YLLVLTK.N | | 425.276 | 2 | 849.545 | 849.544 | 0 | 0.3 | 3041 | 362.9 | 362.9 | 362.9 | 7.03 |
| 1732 | K.NYVALQILQQTFFEGDQQPEIIFGSGFPKDQEYTQLC[+57.021] | C[+57 | 1127.803 | 4 | 4508.191 | 4508.187 | 0 | 0.9 | 3048 | 950.2 | 950.2 | 950.2 | 30.00 |
| | R.N | | | | | | | | | | | | |
| 957 | K.NYVALQILQQTFFEGDQQPEIIFGSGFPK.D | | 1105.563 | 3 | 3314.675 | 3314.673 | 0 | 0.4 | 3048 | 723.2 | 723.2 | 723.2 | 11.75 |
| 1732 | K.NYVALQILQQTFFEGDQQPEIIFGSGFPKDQEYTQLC[+57.021] | C[+57] | 1127.805 | 4 | 4508.197 | 4508.187 | 0 | 2.3 | 3048 | 686.5 | 673.9 | 673.9 | 9.06 |
| | R.N | | | | | | | | | | | | |
| 1732 | K.NYVALQILQQTFFEGDQQPEIIFGSGFPKDQEYTQLC[+57.021] | C[+57] | 902.444 | 5 | 4508.190 | 4508.187 | 0 | 0.7 | 3048 | 573.9 | 573.9 | 573.9 | 8.47 |
| | R.N | | | | | | | | | | | | |
| 957 | K.NYVALQILQQTFFEGDQQPEIIFGSGFPK.D | | 829.424 | 4 | 3314.674 | 3314.673 | 0 | 0.2 | 3048 | 586.0 | 541.0 | 541.0 | 8.15 |
| 1693 | K.DQEYTQLC[+57.021]R.N | C[+57] | 607.270 | 2 | 1213.534 | 1212.531 | 1 | −1.0 | 3077 | 358.0 | 266.8 | 266.8 | 2.86 |
| 1093 | K.YVDLGLGTHR.V | | 565.801 | 2 | 1130.595 | 1130.595 | 0 | −0.5 | 3128 | 670.7 | 670.7 | 670.7 | 11.13 |
| 1806 | R.LIVIEEK.D | | 422.263 | 2 | 843.519 | 843.519 | 00 | 0.1 | 3148 | 365.1 | 365.1 | 365.1 | 6.74 |
| 1806 | R.LIVIEEK.D | | 422.263 | 2 | 843.519 | 843.519 | | 0.0 | 3148 | 302.1 | 302.1 | 302.1 | 5.43 |
| 2000 | K.HYLDINTVLEK.W | | 448.910 | 3 | 1344.716 | 1344.716 | 0 | 0.2 | 3172 | 374.8 | 374.8 | 374.8 | 7.23 |
| 872 | R.ALTEELHQKVSEEAK.S | | 428.727 | 4 | 1711.887 | 1711.886 | 0 | 0.2 | 3241 | 490.7 | 399.5 | 399.5 | 8.53 |
| 872 | R.ALTEELHQKVSEEAK.S | | 428.727 | 4 | 1711.886 | 1711.886 | 0 | −0.2 | 3241 | 569.9 | 569.9 | 569.9 | 8.26 |
| 872 | R.ALTEELHQKVSEEAK.S | | 428.727 | 4 | 1711.885 | 1711.886 | 0 | 0.4 | 3241 | 507.2 | 507.2 | 507.2 | 7.95 |
| 872 | R.ALTEELHQKVSEEAK.S | | 428.727 | 4 | 1711.886 | 1711.886 | 0 | −0.2 | 3241 | 479.6 | 479.6 | 479.6 | 7.79 |
| 872 | R.ALTEELHQKVSEEAK.S | | 428.727 | 4 | 1711.887 | 1711.886 | 0 | 0.2 | 3241 | 477.3 | 477.3 | 477.3 | 7.78 |
| 872 | R.ALTEELHQKVSEEAK.S | | 428.727 | 4 | 1711.887 | 1711.886 | 0 | 0.2 | 3241 | 429.1 | 429.1 | 429.1 | 7.55 |
| 872 | R.ALTEELHQKVSEEAK.S | | 571.300 | 3 | 1711.885 | 1711.886 | 0 | −0.5 | 3241 | 550.7 | 550.7 | 550.7 | 7.53 |
| 872 | R.ALTEELHQKVSEEAK.S | | 428.727 | 4 | 1711.885 | 1711.886 | 0 | 0.4 | 3241 | 451.6 | 451.6 | 451.6 | 7.19 |
| 872 | R.ALTEELHQKVSEEAK.S | | 571.300 | 3 | 1711.886 | 1711.886 | 0 | −0.2 | 3241 | 421.4 | 421.4 | 421.4 | 6.49 |
| 1721 | K.VSEEAKSILLNC[+57.021]ATPDAVVR.L | C[+57] | 724.716 | 3 | 2172.134 | 2172.133 | 0 | 0.5 | 3250 | 448.7 | 448.7 | 448.7 | 8.04 |
| 1721 | K.VSEEAKSILLNC[+57.021]ATPDAVVR.L | C[+57] | 724.716 | 3 | 2172.133 | 2172.133 | 0 | 0.2 | 3250 | 487.1 | 344.0 | 344.0 | 6.96 |
| 1695 | K.SILLNC[+57.021]ATPDAVVR.L | C[+57] | 764.911 | 2 | 1528.815 | 1528.815 | 0 | −0.1 | 3256 | 693.2 | 588.5 | 588.5 | 11.72 |
| 1695 | K.SILLNC[+57.021]ATPDAVVR.L | C[+57] | 510.277 | 3 | 1528.816 | 1528.815 | 0 | 0.2 | 3256 | 445.2 | 445.2 | 445.2 | 7.24 |
| 1695 | K.SILLNC[+57.021]ATPDAVVR.L | C[+57] | 765.412 | 2 | 1529.818 | 1528.815 | 1 | −0.7 | 3256 | 354.5 | 279.9 | 279.9 | 3.35 |
| 1173 | R.LSAYSLGGFAAEWLSQEYFHR.Q | | 811.395 | 3 | 2432.170 | 2432.167 | 0 | 1.1 | 3270 | 875.5 | 875.5 | 875.5 | 15.95 |
| 1173 | R.LSAYSLGGFAAEWLSQEYFHR.Q | | 608.798 | 4 | 2432.169 | 2432.167 | 0 | 0.7 | 3270 | 722.9 | 722.9 | 722.9 | 12.46 |
| 1173 | R.LSAYSLGGFAAEWLSQEYFHR.Q | | 811.394 | 3 | 2432.168 | 2432.167 | 0 | 0.2 | 3270 | 685.7 | 685.7 | 685.7 | 11.98 |
| 1173 | R.LSAYSLGGFAAEWLSQEYFHR.Q | | 811.394 | 3 | 2432.167 | 2432.167 | 0 | 0.1 | 3270 | 625.6 | 625.6 | 625.6 | 10.98 |
| 1173 | R.LSAYSLGGFAAEWLSQEYFHR.Q | | 811.394 | 3 | 2432.167 | 2432.167 | 0 | −0.2 | 3270 | 474.8 | 474.8 | 474.8 | 8.91 |
| 1005 | R.QRHNSFADFLQAHLHTADLER.H | | 502.053 | 5 | 2506.233 | 2506.233 | 0 | 0.0 | 3291 | 578.2 | 578.2 | 578.2 | 9.70 |
| 1005 | R.QRHNSFADFLQAHLHTADLER.H | | 836.083 | 3 | 2506.233 | 2506.233 | 0 | −0.1 | 3291 | 635.5 | 635.5 | 635.5 | 9.28 |
| 1005 | R.QRHNSFADFLQAHLHTADLER.H | | 418.545 | 6 | 2506.234 | 2506.233 | 0 | 0.1 | 3291 | 468.1 | 468.1 | 468.1 | 8.64 |
| 1005 | R.QRHNSFADFLQAHLHTADLER.H | | 502.053 | 5 | 2506.234 | 2506.233 | 0 | 0.2 | 3291 | 464.2 | 464.2 | 464.2 | 8.64 |
| 1005 | R.QRHNSFADFLQAHLHTADLER.H | | 502.053 | 5 | 2506.234 | 2506.233 | 0 | 0.1 | 3291 | 451.3 | 451.3 | 451.3 | 7.75 |
| 1005 | R.QRHNSFADFLQAHLHTADLER.H | | 627.314 | 4 | 2506.235 | 2506.233 | 0 | 0.5 | 3291 | 558.4 | 558.4 | 558.4 | 7.55 |
| 854 | R.HNSFADFLQAHLHTADLER.H | | 445.221 | 5 | 2222.074 | 2222.074 | 0 | 0.0 | 3293 | 564.8 | 564.8 | 564.8 | 9.98 |
| 854 | R.HNSFADFLQAHLHTADLER.H | | 445.221 | 5 | 2222.074 | 2222.074 | 0 | 0.2 | 3293 | 542.0 | 542.0 | 542.0 | 9.25 |
| 854 | R.HNSFADFLQAHLHTADLER.H | | 556.274 | 4 | 2222.074 | 2222.074 | 0 | 0.1 | 3293 | 533.1 | 533.1 | 533.1 | 9.25 |
| 854 | R.HNSFADFLQAHLHTADLER.H | | 556.274 | 4 | 2222.074 | 2222.074 | 0 | −0.1 | 3293 | 540.9 | 540.9 | 540.9 | 8.65 |
| 854 | R.HNSFADFLQAHLHTADLER.H | | 556.274 | 4 | 2222.074 | 2222.074 | 0 | 0.2 | 3293 | 379.9 | 324.2 | 324.2 | 6.26 |
| 943 | R.HAIFTEITTFSR.L | | 711.872 | 2 | 1422.738 | 1422.738 | 0 | 0.0 | 3312 | 558.5 | 558.5 | 558.5 | 8.39 |
| 943 | R.HAIFTEITTFSR.L | | 474.917 | 3 | 1422.738 | 1422.738 | 0 | 0.0 | 3312 | 455.0 | 455.0 | 455.0 | 8.05 |
| 943 | R.HAIFTEITTFSR.L | | 474.917 | 3 | 1422.737 | 1422.738 | 0 | −0.2 | 3312 | 429.4 | 429.4 | 429.4 | 7.83 |
| 943 | R.HAIFTEITTFSR.L | | 474.917 | 3 | 1422.737 | 1422.738 | 0 | −0.1 | 3312 | 371.9 | 371.9 | 371.9 | 7.23 |
| 943 | R.HAIFTEITTFSR.L | | 474.917 | 3 | 1422.738 | 1422.738 | 0 | 0.0 | 3312 | 361.2 | 361.2 | 361.2 | 7.23 |
| 943 | R.HAIFTEITTFSR.L | | 711.873 | 2 | 1422.738 | 1422.738 | 0 | 0.4 | 3312 | 359.7 | 359.7 | 359.7 | 6.09 |
| 1143 | R.APKPTLLWLQQFDTEYSFLKEVR.N | | 937.168 | 3 | 2809.489 | 2809.492 | 0 | 1.0 | 3341 | 705.9 | 705.9 | 705.9 | 12.12 |
| 894 | R.APKPTLLWLQQFDTEYSFLK.E | | 809.099 | 3 | 2425.281 | 2425.280 | 0 | 0.3 | 3341 | 526.5 | 526.5 | 526.5 | 9.25 |
| 1143 | R.APKPTLLWLQQFDTEYSFLKEVR.N | | 703.128 | 4 | 2809.492 | 2809.492 | 0 | −0.1 | 3341 | 548.8 | 548.8 | 548.8 | 8.97 |
| 894 | R.APKPTLLWLQQFDTEYSFLK.E | | 809.098 | 3 | 2425.280 | 2425.280 | 0 | 0.1 | 3341 | 461.3 | 461.3 | 461.3 | 8.31 |
| 1143 | R.APKPTLLWLQQFDTEYSFLKEVR.N | | 703.129 | 4 | 2809.493 | 2809.492 | 0 | 0.2 | 3341 | 442.4 | 442.4 | 442.4 | 8.04 |
| 894 | R.APKPTLLWLQQFDTEYSFLK.E | | 809.433 | 3 | 2426.286 | 2425.280 | 1 | 0.8 | 3341 | 423.0 | 423.0 | 423.0 | 7.80 |
| 894 | R.APKPTLLWLQQFDTEYSFLK.E | | 607.076 | 4 | 2425.280 | 2425.280 | 0 | 0.0 | 3341 | 405.5 | 405.5 | 405.5 | 7.28 |
| 1143 | R.APKPTLLWLQQFDTEYSFLKEVR.N | | 703.129 | 4 | 2809.494 | 2809.492 | 0 | 0.5 | 3341 | 382.8 | 382.8 | 382.8 | 7.22 |
| 2170 | K.ILIFQTDFEDGIR.S | | 783.912 | 2 | 1566.817 | 1566.816 | 0 | 0.4 | 3374 | 526.6 | 526.6 | 526.6 | 8.98 |
| 2170 | K.ILIFQTDFEDGIR.S | | 783.912 | 2 | 1566.817 | 1566.816 | 0 | 0.4 | 3374 | 469.5 | 469.5 | 469.5 | 8.05 |
| 2170 | K.ILIFQTDFEDGIR.S | | 783.912 | 2 | 1566.817 | 1566.816 | 0 | 0.1 | 3374 | 431.8 | 431.8 | 431.8 | 7.53 |
| 848 | K.YSVINEINK.I | | 540.290 | 2 | 1079.573 | 1079.573 | 0 | −0.4 | 3396 | 481.2 | 481.2 | 481.2 | 7.33 |
| 1965 | R.IFVYFITK.L | | 515.798 | 2 | 1030.588 | 1030.597 | 0 | 8.5 | 3412 | 360.5 | 360.5 | 360.5 | 5.18 |
| 855 | R.RSTLMVSDVTR.L | | 422.228 | 3 | 1264.668 | 1264.668 | 0 | 0.1 | 3447 | 475.5 | 475.5 | 475.5 | 7.76 |
| 855 | R.RSTLMVSDVTR.L | | 422.228 | 3 | 1264.668 | 1264.668 | 0 | 0.2 | 3447 | 467.3 | 467.3 | 467.3 | 7.57 |
| 1581 | R.RSTLM[+15.995]VSDVTR.L | M[+16] | 427.559 | 3 | 1280.662 | 1280.663 | 0 | −0.2 | 3447 | 404.2 | 404.2 | 404.2 | 7.35 |
| 1242 | R.STLMVSDVTRLQHVTISQLFAPGDLPELGLEHR.A | | 915.736 | 4 | 3659.922 | 3659.921 | 0 | 0.3 | 3448 | 638.1 | 638.1 | 638.1 | 9.99 |
| 1242 | R.STLMVSDVTRLQHVTISQLFAPGDLPELGLEHR.A | | 732.790 | 5 | 3659.923 | 3659.921 | 0 | 0.4 | 3448 | 622.5 | 622.5 | 622.5 | 9.99 |
| 845 | R.STLMVSDVTR.L | | 554.787 | 2 | 1108.567 | 1108.567 | 0 | −0.2 | 3448 | 551.2 | 551.2 | 551.2 | 7.80 |
| 1579 | R.STLM[+15.995]VSDVTR.L | M[+16] | 562.784 | 2 | 1124.561 | 1124.562 | 0 | −0.6 | 3448 | 472.8 | 472.8 | 472.8 | 7.47 |
| 1579 | R.STLM[+15.995]VSDVTR.L | M[+16] | 562.784 | 2 | 1124.562 | 1124.562 | 0 | 0.0 | 3448 | 385.5 | 363.2 | 363.2 | 7.23 |
| 1579 | R.STLM[+15.995]VSDVTR.L | M[+16] | 562.784 | 2 | 1124.561 | 1124.562 | 0 | −0.5 | 3448 | 458.8 | 458.8 | 458.8 | 7.18 |
| 845 | R.STLMVSDVTR.L | | 554.787 | 2 | 1108.567 | 1108.567 | 0 | −0.2 | 3448 | 373.1 | 373.1 | 373.1 | 6.64 |
| 845 | R.STLMVSDVTR.L | | 554.787 | 2 | 1108.566 | 1108.567 | 0 | −0.4 | 3448 | 490.6 | 313.2 | 313.2 | 5.71 |
| 979 | R.LQHVTISQLFAPGDLPELGLEHR.A | | 643.349 | 4 | 2570.374 | 2570.373 | 0 | 0.5 | 3458 | 724.0 | 724.0 | 724.0 | 12.98 |
| 1391 | R.LQHVTISQLFAPGDLPELGLEHRAEDGHEEAMETEASTSGEVAEVA | | 920.150 | 7 | 6435.006 | 6435.004 | 0 | 0.3 | 3458 | 766.0 | 766.0 | 766.0 | 11.88 |
| | EEAMETESSEKVGK.E | | | | | | | | | | | | |
| 979 | R.LQHVTISQLFAPGDLPELGLEHR.A | | 857.462 | 3 | 2570.373 | 2570.373 | 0 | 0.0 | 3458 | 644.7 | 644.7 | 644.7 | 10.98 |
| 1391 | R.LQHVTISQLFAPGDLPELGLEHRAEDGHEEAMETEASTSGEVAEVA | | 920.149 | 7 | 6435.001 | 6435.004 | 0 | −0.5 | 3458 | 778.8 | 778.8 | 778.8 | 10.41 |
| | EEAMETESSEKVGK.E | | | | | | | | | | | | |
| 1391 | R.LQHVTISQLFAPGDLPELGLEHRAEDGHEEAMETEASTSGEVAEVA | | 805.257 | 8 | 6435.004 | 6435.004 | 0 | 0.0 | 3458 | 682.4 | 682.4 | 682.4 | 8.77 |
| | EEAMETESSEKVGK.E | | | | | | | | | | | | |
| 979 | R.LQHVTISQLFAPGDLPELGLEHR.A | | 643.349 | 4 | 2570.375 | 2570.373 | 0 | 0.9 | 3458 | 428.6 | 428.6 | 428.6 | 8.68 |
| 1391 | R.LQHVTISQLFAPGDLPELGLEHRAEDGHEEAMETEASTSGEVAEVA | | 920.293 | 7 | 6436.008 | 6435.004 | 1 | 0.1 | 3458 | 605.4 | 605.4 | 605.4 | 8.28 |
| | EEAMETESSEKVGK.E | | | | | | | | | | | | |
| 1391 | R.LQHVTISQLFAPGDLPELGLEHRAEDGHEEAMETEASTSGEVAEVA | | 920.149 | 7 | 6434.999 | 6435.004 | 0 | 0.8 | 3458 | 632.1 | 632.1 | 632.1 | 7.41 |
| | EEAMETESSEKVGK.E | | | | | | | | | | | | |
| 835 | R.LQHVTISQLFAPGDLPELGLEHRAEDGHEEAMETEASTSGEVAEVA | | 1230.971 | 5 | 6150.825 | 6150.819 | 0 | 1.0 | 3458 | 579.8 | 579.8 | 579.8 | 7.34 |
| | EEAMETESSEK.V | | | | | | | | | | | | |
| 835 | R.LQHVTISQLFAPGDLPELGLEHRAEDGHEEAMETEASTSGEVAEVA | | 1025.975 | 6 | 6150.813 | 6150.819 | 0 | −1.0 | 3458 | 521.1 | 521.1 | 521.1 | 5.84 |
| | EEAMETESSEK.V | | | | | | | | | | | | |
| 835 | R.LQHVTISQLFAPGDLPELGLEHRAEDGHEEAMETEASTSGEVAEVA | | 879.696 | 7 | 6151.826 | 6150.819 | 1 | 0.5 | 3458 | 467.5 | 370.9 | 370.9 | 5.37 |
| | EEAMETESSEK.V | | | | | | | | | | | | |
| 954 | R.AEDGHEEAMETEASTSGEVAEVAEEAMETESSEKVGKETSELGGSD | | 1129.704 | 5 | 5644.488 | 5644.492 | 0 | −0.6 | 3481 | 910.7 | 910.7 | 910.7 | 14.30 |
| | VSILDTTR.L | | | | | | | | | | | | |
| 954 | R.AEDGHEEAMETEASTSGEVAEVAEEAMETESSEKVGKETSELGGSD | | 1129.706 | 5 | 5644.502 | 5644.492 | 0 | 1.8 | 3481 | 736.9 | 736.9 | 736.9 | 10.88 |
| | VSILDTTR.L | | | | | | | | | | | | |
| 954 | R.AEDGHEEAMETEASTSGEVAEVAEEAMETESSEKVGKETSELGGSD | | 1129.705 | 5 | 5644.495 | 5644.492 | 0 | 0.4 | 3481 | 733.2 | 733.2 | 733.2 | 10.88 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.490 | 5644.492 | 0 | −0.4 | 3481 | 767.4 | 767.4 | 767.4 | 10.70 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.491 | 5644.492 | 0 | −0.2 | 3481 | 727.1 | 727.1 | 727.1 | 10.30 |
| | VSILDTTR.L | | | | | | | | | | | | |
| 948 | R.AEDGHEEAMETEASTSGEVAEVAEEAMETESSEK.V | | 1200.493 | 3 | 3599.465 | 3599.464 | 0 | 0.2 | 3481 | 603.2 | 603.2 | 603.2 | 10.26 |
| 939 | R.AEDGHEEAMETEASTSGEVAEVAEEAMETESSEKVGK.E | | 971.668 | 4 | 3883.649 | 3883.649 | 0 | 0.0 | 3481 | 609.6 | 609.6 | 609.6 | 9.99 |
| 954 | R.AEDGHEEAMETEASTSGEVAEVAEEAMETESSEKVGKETSELGGSD | | 1129.905 | 5 | 5645.497 | 5644.492 | 1 | 0.2 | 3481 | 669.7 | 669.7 | 669.7 | 8.99 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.491 | 5644.492 | 0 | −0.1 | 3481 | 633.1 | 633.1 | 633.1 | 8.88 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.491 | 5644.492 | 0 | −0.2 | 3481 | 661.0 | 661.0 | 661.0 | 8.70 |
| | VSILDTTR.L | | | | | | | | | | | | |
| 954 | R.AEDGHEEAMETEASTSGEVAEVAEEAMETESSEKVGKETSELGGSD | | 1411.877 | 4 | 5644.488 | 5644.492 | 0 | 0.7 | 3481 | 647.9 | 647.9 | 647.9 | 8.30 |
| | VSILDTTR.L | | | | | | | | | | | | |
| 954 | R.AEDGHEEAMETEASTSGEVAEVAEEAMETESSEKVGKETSELGGSD | | 1130.105 | 5 | 5646.496 | 5644.492 | 2 | −0.4 | 3481 | 628.0 | 628.0 | 628.0 | 8.30 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.491 | 5644.492 | 0 | −0.1 | 3481 | 629.8 | 629.8 | 629.8 | 8.28 |
| | VSILDTTR.L | | | | | | | | | | | | |
| 948 | R.AEDGHEEAMETEASTSGEVAEVAEEAMETESSEK.V | | 1200.494 | 3 | 3599.468 | 3599.464 | 0 | 0.9 | 3481 | 559.8 | 559.8 | 559.8 | 7.94 |
| 954 | R.AEDGHEEAMETEASTSGEVAEVAEEAMETESSEKVGKETSELGGSD | | 1129.705 | 5 | 5644.493 | 5644.492 | 0 | 0.2 | 3481 | 588.8 | 588.8 | 588.8 | 7.88 |
| | VSILDTTR.L | | | | | | | | | | | | |
| 954 | R.AEDGHEEAMETEASTSGEVAEVAEEAMETESSEKVGKETSELGGSD | | 1411.878 | 4 | 5644.492 | 5644.492 | 0 | 0.0 | 3481 | 572.3 | 572.3 | 572.3 | 7.88 |
| | VSILDTTR.L | | | | | | | | | | | | |
| 948 | R.AEDGHEEAMETEASTSGEVAEVAEEAMETESSEK.V | | 1201.161 | 3 | 3601.469 | 3599.464 | 2 | 0.7 | 3481 | 565.6 | 503.3 | 503.3 | 7.79 |
| 948 | R.AEDGHEEAMETEASTSGEVAEVAEEAMETESSEK.V | | 1200.492 | 3 | 3599.462 | 3599.464 | 0 | −0.7 | 3481 | 534.9 | 534.9 | 534.9 | 7.06 |
| 948 | R.AEDGHEEAMETEASTSGEVAEVAEEAMETESSEK.V | | 1200.493 | 3 | 3599.463 | 3599.464 | 0 | −0.3 | 3481 | 528.6 | 528.6 | 528.6 | 7.06 |
| 948 | R.AEDGHEEAMETEASTSGEVAEVAEEAMETESSEK.V | | 1201.161 | 3 | 3601.469 | 3599.464 | 2 | −0.5 | 3481 | 490.4 | 490.4 | 490.4 | 6.88 |
| 954 | R.AEDGHEEAMETEASTSGEVAEVAEEAMETESSEKVGKETSELGGSD | | 1130.106 | 5 | 5646.501 | 5644.492 | 2 | 0.3 | 3481 | 562.8 | 562.8 | 562.8 | 6.81 |
| | VSILDTTR.L | | | | | | | | | | | | |
| 948 | R.AEDGHEEAMETEASTSGEVAEVAEEAMETESSEK.V | | 1200.492 | 3 | 3599.462 | 3599.464 | 0 | −0.6 | 3481 | 463.3 | 463.3 | 463.3 | 6.54 |
| 954 | R.AEDGHEEAMETEASTSGEVAEVAEEAMETESSEKVGKETSELGGSD | | 1129.906 | 5 | 5645.499 | 5644.492 | 1 | 0.6 | 3481 | 529.5 | 529.5 | 529.5 | 6.26 |
| | VSILDTTR.L | | | | | | | | | | | | |
| 948 | R.AEDGHEEAMETEASTSGEVAEVAEEAMETESSEK.V | | 1200.491 | 3 | 3599.459 | 3599.464 | 0 | −1.4 | 3481 | 392.4 | 392.4 | 392.4 | 6.00 |
| 954 | R.AEDGHEEAMETEASTSGEVAEVAEEAMETESSEKVGKETSELGGSD | | 1129.704 | 5 | 5644.491 | 5644.492 | 0 | −0.2 | 3481 | 528.2 | 528.2 | 528.2 | 5.97 |
| | VSILDTTR.L | | | | | | | | | | | | |
| 954 | R.AEDGHEEAMETEASTSGEVAEVAEEAMETESSEKVGKETSELGGSD | | 1129.705 | 5 | 5644.497 | 5644.492 | 0 | 0.9 | 3481 | 480.5 | 480.5 | 480.5 | 5.70 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.488 | 5644.492 | 0 | −0.6 | 3481 | 528.1 | 528.1 | 528.1 | 5.68 |
| | VSILDTTR.L | | | | | | | | | | | | |
| 954 | R.AEDGHEEAMETEASTSGEVAEVAEEAMETESSEKVGKETSELGGSD | | 941.924 | 6 | 5646.508 | 5644.492 | 2 | 1.7 | 3481 | 529.0 | 529.0 | 529.0 | 5.36 |
| | VSILDTTR.L | | | | | | | | | | | | |
| 954 | R.AEDGHEEAMETEASTSGEVAEVAEEAMETESSEKVGKETSELGGSD | | 941.588 | 6 | 5644.489 | 5644.492 | 0 | −0.5 | 3481 | 548.8 | 548.8 | 548.8 | 4.97 |
| | VSILDTTR.L | | | | | | | | | | | | |
| 954 | R.AEDGHEEAMETEASTSGEVAEVAEEAMETESSEKVGKETSELGGSD | | 941.588 | 6 | 5644.491 | 5644.492 | 0 | −0.2 | 3481 | 496.0 | 496.0 | 496.0 | 4.79 |
| | VSILDTTR.L | | | | | | | | | | | | |
| 954 | R.AEDGHEEAMETEASTSGEVAEVAEEAMETESSEKVGKETSELGGSD | | 941.587 | 6 | 5644.486 | 5644.492 | 0 | −1.0 | 3481 | 491.1 | 491.1 | 491.1 | 4.79 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.491 | 5644.492 | 0 | −0.2 | 3481 | 390.8 | 390.8 | 390.8 | 4.32 |
| | VSILDTTR.L | | | | | | | | | | | | |
| 954 | R.AEDGHEEAMETEASTSGEVAEVAEEAMETESSEKVGKETSELGGSD | | 1129.704 | 5 | 5644.488 | 5644.492 | 0 | −0.6 | 3481 | 363.7 | 363.7 | 363.7 | 4.28 |
| | VSILDTTR.L | | | | | | | | | | | | |
| 954 | R.AEDGHEEAMETEASTSGEVAEVAEEAMETESSEKVGKETSELGGSD | | 1129.706 | 5 | 5644.502 | 5644.492 | 0 | 1.7 | 3481 | 348.3 | 348.3 | 348.3 | 3.58 |
| | VSILDTTR.L | | | | | | | | | | | | |
| 948 | R.AEDGHEEAMETEASTSGEVAEVAEEAMETESSEK.V | | 900.621 | 4 | 3599.463 | 3599.464 | 0 | 0.2 | 3481 | 304.0 | 304.0 | 304.0 | 3.58 |
| 988 | K.VGKETSELGGSDVSILDTTR.L | | 1032.526 | 2 | 2064.045 | 2064.046 | 0 | −0.2 | 3515 | 631.2 | 631.2 | 631.2 | 10.70 |
| 988 | K.VGKETSELGGSDVSILDTTR.L | | 688.687 | 3 | 2064.046 | 2064.046 | 0 | 0.1 | 3515 | 515.2 | 515.2 | 515.2 | 8.79 |
| 827 | K.ETSELGGSDVSILDTTR.L | | 890.434 | 2 | 1779.861 | 1779.861 | 0 | 0.1 | 3518 | 718.0 | 718.0 | 718.0 | 12.98 |
| 827 | K.ETSELGGSDVSILDTTR.L | | 890.433 | 2 | 1779.860 | 1779.861 | 0 | −0.6 | 3518 | 724.1 | 724.1 | 724.1 | 12.39 |
| 827 | K.ETSELGGSDVSILDTTR.L | | 890.433 | 2 | 1779.860 | 1779.861 | 0 | −0.6 | 3518 | 570.3 | 570.3 | 570.3 | 9.39 |
| 1679 | R.SC[+57.021]VQSAVGMLRDQNESC[+57.021]TR.N | C[+57]* | 733.330 | 3 | 2197.976 | 2197.975 | 0 | 0.7 | 3538 | 546.8 | 546.8 | 546.8 | 8.97 |
| | | 2 | | | | | | | | | | | |
| 1713 | R.SC[+57.021]VQSAVGMLR.D | C[+57] | 604.300 | 2 | 1207.592 | 1207.592 | 0 | −0.1 | 3538 | 513.6 | 417.4 | 417.4 | 8.21 |
| 1679 | R.SC[+57.021]VQSAVGMLRDQNESC[+57.021]TR.N | C[+57]* | 733.330 | 3 | 2197.976 | 2197.975 | 0 | 0.4 | 3538 | 498.0 | 498.0 | 498.0 | 8.20 |
| | | 2 | | | | | | | | | | | |
| 1713 | R.SC[+57.021]VQSAVGMLR.D | C[+57] | 604.299 | 2 | 1207.592 | 1207.592 | 0 | −0.5 | 3538 | 506.2 | 506.2 | 506.2 | 7.62 |
| 1727 | R.VVLLLGLLNEDDAC[+57.021]HASFLR.V | C[+57] | 752.400 | 3 | 2255.187 | 2255.185 | 0 | 0.6 | 3561 | 595.1 | 595.1 | 595.1 | 9.98 |
| 828 | R.LSVFLKKQEESQFHPLEWLAR.E | | 647.103 | 4 | 2585.390 | 2585.388 | 0 | 0.8 | 3586 | 394.4 | 382.0 | 382.0 | 6.87 |
| 828 | R.LSVFLKKQEESQFHPLEWLAR.E | | 517.883 | 5 | 2585.388 | 2585.388 | 0 | 0.1 | 3586 | 327.0 | 327.0 | 327.0 | 5.55 |
| 1038 | K.KQEESQFHPLEWLAR.E | | 475.245 | 4 | 1897.956 | 1897.956 | 0 | 0.5 | 3592 | 388.0 | 388.0 | 388.0 | 7.42 |
| 1038 | K.KQEESQFHPLEWLAR.E | | 475.244 | 4 | 1897.956 | 1897.956 | 0 | 0.2 | 3592 | 347.2 | 347.2 | 347.2 | 5.91 |
| 1038 | K.KQEESQFHPLEWLAR.E | | 475.244 | 4 | 1897.956 | 1897.956 | 0 | 0.2 | 3592 | 326.9 | 326.9 | 326.9 | 5.91 |
| 859 | K.QEESQFHPLEWLAR.E | | 590.625 | 3 | 1769.861 | 1769.861 | 0 | 0.0 | 3593 | 547.7 | 547.7 | 547.7 | 8.98 |
| 859 | K.QEESQFHPLEWLAR.E | | 885.434 | 2 | 1769.860 | 1769.861 | 0 | −0.1 | 3593 | 498.3 | 498.3 | 498.3 | 8.80 |
| 1739 | R.EAC[+57.021]NQDALQEAGTFR.H | C[+57] | 855.381 | 2 | 1709.754 | 1709.755 | 0 | −0.4 | 3607 | 673.6 | 673.6 | 673.6 | 11.13 |
| 2234 | R.VQGAVTPLLASMISFIDR.D | | 640.021 | 3 | 1918.048 | 1918.047 | 0 | 0.8 | 3628 | 423.6 | 423.6 | 423.6 | 7.57 |
| 1524 | R.DGNLELLT[+79.966]RPDTPPWARDLWM[+15.995]FIFS | M[+16], | 821.054 | 6 | 4921.289 | 4920.300 | 1 | 2.9 | 3646 | 374.6 | 241.2 | 15.8 | 2.64 |
| | [+79.966]DTMLLNIPLVMNNER.H | S[+80], | | | | | | | | | | | |
| | | T[+80] | | | | | | | | | | | |
| 1094 | K.IKDYLEELWVQAQYITDAEGLPKK.F | | 713.379 | 4 | 2850.495 | 2850.492 | 0 | 0.8 | 3715 | 677.8 | 677.8 | 677.8 | 11.36 |
| 1214 | K.IKDYLEELWVQAQYITDAEGLPK.K | | 908.479 | 3 | 2723.421 | 2722.397 | 1 | 7.5 | 3715 | 442.7 | 442.7 | 442.7 | 6.19 |
| 1856 | K.KFVDIFQQTPLGR.F | | 774.930 | 2 | 1548.853 | 1548.853 | 0 | 0.5 | 3738 | 542.6 | 542.6 | 542.6 | 8.40 |
| 1856 | K.KFVDIFQQTPLGR.F | | 516.956 | 3 | 1548.853 | 1548.853 | 0 | 0.4 | 3738 | 407.3 | 407.3 | 407.3 | 6.76 |
| 1856 | K.KFVDIFQQTPLGR.F | | 516.956 | 3 | 1548.853 | 1548.853 | 0 | 0.3 | 3738 | 318.6 | 318.6 | 318.6 | 5.04 |
| 1379 | R.KLKAASEAPEEEVSLPWVHLAYQR.F | | 688.617 | 4 | 2751.445 | 2751.446 | 0 | 0.6 | 3797 | 683.9 | 683.9 | 683.9 | 11.12 |
| 1379 | R.KLKAASEAPEEEVSLPWVHLAYQR.F | | 688.618 | 4 | 2751.448 | 2751.446 | 0 | 0.6 | 3797 | 574.5 | 574.5 | 574.5 | 9.70 |
| 1167 | K.LKAASEAPEEEVSLPWVHLAYQR.F | | 656.594 | 4 | 2623.352 | 2623.352 | 0 | 0.3 | 3798 | 607.0 | 607.0 | 607.0 | 10.70 |
| 1167 | K.LKAASEAPEEEVSLPWVHLAYQR.F | | 875.122 | 3 | 2623.353 | 2623.352 | 0 | 0.4 | 3798 | 569.4 | 569.4 | 569.4 | 9.11 |
| 1167 | K.LKAASEAPEEEVSLPWVHLAYQR.F | | 875.122 | 3 | 2623.351 | 2623.352 | 0 | −0.3 | 3798 | 545.0 | 545.0 | 545.0 | 8.39 |
| 1167 | K.LKAASEAPEEEVSLPWVHLAYQR.F | | 525.476 | 5 | 2623.351 | 2623.352 | 0 | −0.4 | 3798 | 530.1 | 521.1 | 521.1 | 7.87 |
| 1273 | K.AASEAPEEEVSLPWVHLAYQR.F | | 794.729 | 3 | 2382.172 | 2382.173 | 0 | 0.0 | 3800 | 801.8 | 801.8 | 801.8 | 14.95 |
| 1273 | K.AASEAPEEEVSLPWVHLAYQR.F | | 1191.590 | 2 | 2382.172 | 2382.173 | 0 | −0.3 | 3800 | 604.0 | 604.0 | 604.0 | 10.39 |
| 947 | R.ILTIYPQVLHSLMEAR.W | | 942.525 | 2 | 1884.042 | 1884.041 | 0 | 0.4 | 3831 | 559.1 | 523.2 | 523.2 | 8.98 |
| 947 | R.ILTIYPQVLHSLMEAR.W | | 628.686 | 3 | 1884.043 | 1884.041 | 0 | 0.7 | 3831 | 507.5 | 507.5 | 507.5 | 8.80 |
| 1588 | R.ILTIYPQVLHSLM[+15.995]EAR.W | M[+16] | 634.017 | 3 | 1900.037 | 1900.036 | 0 | 0.7 | 3831 | 465.8 | 465.8 | 465.8 | 8.65 |
| 947 | R.ILTIYPQVLHSLMEAR.W | | 628.686 | 3 | 1884.043 | 1884.041 | 0 | 0.7 | 3831 | 379.1 | 379.1 | 379.1 | 8.30 |
| 1588 | R.ILTIYPQVLHSLM[+15.995]EAR.W | M[+16] | 634.017 | 3 | 1900.037 | 1900.036 | 0 | 0.7 | 3831 | 391.3 | 391.3 | 391.3 | 7.71 |
| 947 | R.ILTIYPQVLHSLMEAR.W | | 628.686 | 3 | 1884.042 | 1884.041 | 0 | 0.6 | 3831 | 351.1 | 351.1 | 351.1 | 6.38 |
| 992 | R.NTLKPSPQAWLQLVK.N | | 575.002 | 3 | 1722.990 | 1722.990 | 0 | 0.2 | 3873 | 648.6 | 648.6 | 648.6 | 10.72 |
| 992 | R.NTLKPSPQAWLQLVK.N | | 575.002 | 3 | 1722.990 | 1722.990 | 0 | 0.1 | 3873 | 604.0 | 604.0 | 604.0 | 10.72 |
| 992 | R.NTLKPSPQAWLQLVK.N | | 861.998 | 2 | 1722.989 | 1722.990 | 0 | −0.7 | 3873 | 495.4 | 495.4 | 495.4 | 8.22 |
| 992 | R.NTLKPSPQAWLQLVK.N | | 575.335 | 3 | 1723.992 | 1722.990 | 1 | −1.0 | 3873 | 473.0 | 473.0 | 473.0 | 7.47 |
| 992 | R.NTLKPSPQAWLQLVK.N | | 575.001 | 3 | 1722.989 | 1722.990 | 0 | −0.5 | 3873 | 416.2 | 416.2 | 416.2 | 6.95 |
| 992 | R.NTLKPSPQAWLQLVK.N | | 431.503 | 4 | 1722.990 | 1722.990 | 0 | 0.0 | 3873 | 307.3 | 307.3 | 307.3 | 5.11 |
| 2814 | R.IFSTALFVEHVLLGTESR.V | | 673.702 | 3 | 2019.092 | 2019.091 | 0 | 0.7 | 3923 | 597.4 | 597.4 | 597.4 | 9.98 |
| 1047 | R.VPELQGLVTEHVFLLDK.C | | 646.363 | 3 | 1937.075 | 1937.074 | 0 | 0.2 | 3941 | 466.1 | 466.1 | 466.1 | 8.91 |
| 1047 | R.VPELQGLVTEHVFLLDK.C | | 646.364 | 3 | 1937.077 | 1937.074 | 0 | 1.2 | 3941 | 336.7 | 336.7 | 336.7 | 6.17 |
| 1090 | R.ENSDVKTHGPFEAVMR.T | | 606.293 | 3 | 1816.864 | 1816.865 | 0 | 0.3 | 3961 | 618.8 | 618.8 | 618.8 | 9.86 |
| 1090 | R.ENSDVKTHGPFEAVMR.T | | 606.293 | 3 | 1816.864 | 1816.865 | 0 | 0.3 | 3961 | 579.2 | 579.2 | 579.2 | 8.86 |
| 1090 | R.ENSDVKTHGPFEAVMR.T | | 606.293 | 3 | 1816.864 | 1816.865 | 0 | 0.5 | 3961 | 568.5 | 568.5 | 568.5 | 8.86 |
| 1090 | R.ENSDVKTHGPFEAVMR.T | | 454.972 | 4 | 1816.865 | 1816.865 | 0 | 0.0 | 3961 | 413.1 | 413.1 | 413.1 | 7.26 |
| 1090 | R.ENSDVKTHGPFEAVMR.T | | 454.972 | 4 | 1816.865 | 1816.865 | 0 | −0.1 | 3961 | 394.2 | 394.2 | 394.2 | 6.96 |
| 1090 | R.ENSDVKTHGPFEAVMR.T | | 909.437 | 2 | 1817.867 | 1816.865 | 1 | −0.4 | 3961 | 479.6 | 479.6 | 479.6 | 6.37 |
| 1090 | R.ENSDVKTHGPFEAVMR.T | | 454.972 | 4 | 1816.865 | 1816.865 | 0 | 0.2 | 3961 | 332.7 | 332.7 | 332.7 | 5.63 |
| 825 | K.THGPFEAVMR.T | | 572.782 | 2 | 1144.557 | 1144.557 | 0 | 0.1 | 3967 | 545.6 | 545.6 | 545.6 | 8.39 |
| 825 | K.THGPFEAVMR.T | | 572.782 | 2 | 1144.557 | 1144.557 | 0 | 0.1 | 3967 | 509.8 | 509.8 | 509.8 | 7.92 |
| 825 | K.THGPFEAVMR.T | | 572.782 | 2 | 1144.557 | 1144.557 | 0 | −0.1 | 3967 | 479.5 | 479.5 | 479.5 | 7.76 |
| 825 | K.THGPFEAVMR.T | | 572.782 | 2 | 1144.557 | 1144.557 | 0 | 0.2 | 3967 | 476.3 | 476.3 | 476.3 | 7.18 |
| 825 | K.THGPFEAVMR.T | | 572.783 | 2 | 1144.560 | 1144.557 | 0 | 2.4 | 3967 | 415.9 | 415.9 | 415.9 | 5.94 |
| 1681 | R.AWFASEQMIC[+57.021]PYC[+57.021]LTALPDEFSPAVSQA | C[+57]* | 1161.538 | 3 | 3482.599 | 3482.597 | 0 | 0.7 | 4023 | 739.9 | 739.9 | 739.9 | 12.26 |
| | HR.E | 2 | | | | | | | | | | | |
| 1681 | R.AWFASEQMIC[+57.021]PYC[+57.021]LTALPDEFSPAVSQA | C[+57]* | 871.405 | 4 | 3482.599 | 3482.597 | 0 | 0.7 | 4023 | 566.5 | 541.5 | 541.5 | 7.86 |
| | HR.E | 2 | | | | | | | | | | | |
| 856 | K.DNAPPEKEVIESLLSLLFVQK.G | | 790.438 | 3 | 2369.298 | 2369.296 | 0 | 0.9 | 4080 | 545.9 | 545.9 | 545.9 | 8.97 |
| 856 | K.DNAPPEKEVIESLLSLLFVQK.G | | 593.080 | 4 | 2369.297 | 2369.296 | 0 | 0.4 | 4080 | 406.6 | 406.6 | 406.6 | 7.30 |
| 2072 | K.SLSPFNDVVDKTPVIR.S | | 893.988 | 2 | 1786.969 | 1786.970 | 0 | 0.2 | 4116 | 384.3 | 384.3 | 384.3 | 6.85 |
| 2072 | K.SLSPFNDVVDKTPVIR.S | | 893.988 | 2 | 1786.969 | 1786.970 | 0 | 0.3 | 4116 | 388.5 | 388.5 | 388.5 | 6.56 |
| 1780 | K.AFITEDK.T | | 412.210 | 2 | 823.413 | 823.420 | 0 | −7.9 | 4161 | 325.8 | 325.8 | 325.8 | 4.05 |
| 1780 | K.AFITEDK.T | | 412.210 | 2 | 823.413 | 823.420 | 0 | 7.5 | 4161 | 302.6 | 302.6 | 302.6 | 3.77 |
| 1408 | K.TSAYSRNDELNHLEEEGR.F | | 707.326 | 3 | 2119.964 | 2119.964 | 0 | −0.1 | 4186 | 732.9 | 732.9 | 732.9 | 12.70 |
| 936 | K.TSAYSRNDELNHLEEEGRFLK.A | | 627.808 | 4 | 2508.211 | 2508.211 | 0 | 0.1 | 4186 | 473.4 | 473.4 | 473.4 | 8.29 |
| 936 | K.TSAYSRNDELNHLEEEGRFLK.A | | 502.448 | 5 | 2508.212 | 2508.211 | 0 | 0.1 | 4186 | 414.7 | 414.7 | 414.7 | 8.06 |
| 1408 | K.TSAYSRNDELNHLEEEGR.F | | 530.746 | 4 | 2119.964 | 2119.964 | 0 | 0.1 | 4186 | 380.9 | 380.9 | 380.9 | 7.70 |
| 936 | K.TSAYSRNDELNHLEEEGRFLK.A | | 502.649 | 5 | 2509.215 | 2508.211 | 1 | 0.0 | 4186 | 373.7 | 373.7 | 94.8 | 7.06 |
| 1408 | K.TSAYSRNDELNHLEEEGR.F | | 1060.484 | 2 | 2119.961 | 2119.964 | 0 | −1.6 | 4186 | 397.7 | 397.7 | 397.7 | 6.29 |
| 1408 | K.TSAYSRNDELNHLEEEGR.F | | 530.746 | 4 | 2119.964 | 2119.964 | 0 | 0.1 | 4186 | 351.9 | 351.9 | 351.9 | 6.08 |
| 936 | K.TSAYSRNDELNHLEEEGRFLK.A | | 836.742 | 3 | 2508.210 | 2508.211 | 0 | 0.4 | 4186 | 420.2 | 420.2 | 420.2 | 5.17 |
| 1482 | K.TSAYSRNDELN[+0.984]HLEEEGRFLK.A | N[+1] | 502.645 | 5 | 2509.197 | 2509.195 | 0 | 0.6 | 4186 | 311.4 | 311.4 | 20.7 | 5.07 |
| 807 | R.NDELNHLEEEGR.F | | 727.829 | 2 | 1454.651 | 1454.651 | 0 | 0.2 | 4192 | 792.6 | 792.6 | 792.6 | 13.72 |
| 1494 | R.N[+0.984]DELNHLEEEGRFLK.A | N[+1] | 615.299 | 3 | 1843.883 | 1843.882 | 0 | 0.3 | 4192 | 686.1 | 686.1 | 277.8 | 11.44 |
| 978 | R.NDELNHLEEEGRFLK.A | | 614.971 | 3 | 1842.898 | 1842.898 | 0 | −0.1 | 4192 | 574.6 | 574.6 | 574.6 | 9.44 |
| 978 | R.NDELNHLEEEGRFLK.A | | 614.971 | 3 | 1842.899 | 1842.898 | 0 | 0.6 | 4192 | 530.5 | 530.5 | 530.5 | 8.71 |
| 807 | R.NDELNHLEEEGR.F | | 485.555 | 3 | 1454.651 | 1454.651 | 0 | 0.1 | 4192 | 462.1 | 462.1 | 462.1 | 8.65 |
| 978 | R.NDELNHLEEEGRFLK.A | | 461.480 | 4 | 1842.898 | 1842.898 | 0 | 0.1 | 4192 | 448.2 | 448.2 | 448.2 | 8.38 |
| 1496 | R.NDELN[+0.984]HLEEEGR.F | N[+1] | 485.883 | 3 | 1455.635 | 1455.635 | 0 | 0.0 | 4192 | 492.5 | 397.2 | 341.1 | 8.21 |
| 978 | R.NDELNHLEEEGRFLK.A | | 461.480 | 4 | 1842.898 | 1842.898 | 0 | 0.1 | 4192 | 489.2 | 475.8 | 475.8 | 7.94 |
| 1488 | R.NDELN[+0.984]HLEEEGRFLK.A | N[+1] | 461.726 | 4 | 1843.882 | 1843.882 | 0 | 0.3 | 4192 | 479.5 | 467.9 | 169.7 | 7.79 |
| 978 | R.NDELNHLEEEGRFLK.A | | 614.971 | 3 | 1842.898 | 1842.898 | 0 | −0.2 | 4192 | 479.5 | 479.5 | 479.5 | 7.78 |
| 1488 | R.NDELN[+0.984]HLEEEGRFLK.A | N[+1] | 615.299 | 3 | 1843.882 | 1843.882 | 0 | 0.0 | 4192 | 471.6 | 471.6 | 228.4 | 7.78 |
| 1494 | R.N[+0.984]DELNHLEEEGRFLK.A | N[+1] | 461.726 | 4 | 1843.882 | 1843.882 | 0 | 0.1 | 4192 | 444.8 | 444.8 | 104.1 | 7.78 |
| 807 | R.NDELNHLEEEGR.F | | 485.555 | 3 | 1454.650 | 1454.651 | 0 | 0.3 | 4192 | 506.9 | 506.9 | 506.9 | 7.62 |
| 1496 | R.NDELN[+0.984]HLEEEGR.F | N[+1] | 485.883 | 3 | 1455.634 | 1455.635 | 0 | −0.3 | 4192 | 474.5 | 372.7 | 305.1 | 7.47 |
| 807 | R.NDELNHLEEEGR.F | | 727.829 | 2 | 1454.650 | 1454.651 | 0 | 0.3 | 4192 | 471.9 | 471.9 | 471.9 | 7.47 |
| 978 | R.NDELNHLEEEGRFLK.A | | 614.971 | 3 | 1842.898 | 1842.898 | 0 | 0.3 | 4192 | 506.5 | 506.5 | 506.5 | 7.35 |
| 978 | R.NDELNHLEEEGRFLK.A | | 461.731 | 4 | 1843.903 | 1842.898 | 1 | 0.6 | 4192 | 409.5 | 409.5 | 409.5 | 7.26 |
| 1488 | R.NDELN[+0.984]HLEEEGRFLK.A | N[+1] | 461.726 | 4 | 1843.882 | 1843.882 | 0 | 0.1 | 4192 | 382.5 | 382.5 | 100.9 | 7.14 |
| 978 | R.NDELNHLEEEGRFLK.A | | 461.480 | 4 | 1842.898 | 1842.898 | 0 | 0.3 | 4192 | 438.5 | 438.5 | 438.5 | 6.68 |
| 978 | R.NDELNHLEEEGRFLK.A | | 921.953 | 2 | 1842.898 | 1842.898 | 0 | 0.0 | 4192 | 439.1 | 439.1 | 439.1 | 6.13 |
| 1496 | R.NDELN[+0.984]HLEEEGR.F | N[+1] | 728.822 | 2 | 1456.637 | 1455.635 | 1 | 0.7 | 4192 | 447.8 | 447.8 | 151.5 | 5.31 |
| 2409 | R.FLKAYSPASR.G | | 570.314 | 2 | 1139.621 | 1139.621 | 0 | 0.2 | 4204 | 498.1 | 498.1 | 498.1 | 7.64 |
| 831 | R.GREPANEASVEYLQEVAR.I | | 673.336 | 3 | 2017.994 | 2017.994 | 0 | 0.1 | 4214 | 495.9 | 495.9 | 495.9 | 8.79 |
| 831 | R.GREPANEASVEYLQEVAR.I | | 1009.500 | 2 | 2017.993 | 2017.994 | 0 | 0.2 | 4214 | 401.8 | 401.8 | 401.8 | 7.81 |
| 831 | R.GREPANEASVEYLQEVAR.I | | 673.336 | 3 | 2017.993 | 2017.994 | 0 | −0.3 | 4214 | 493.5 | 493.5 | 493.5 | 7.61 |
| 831 | R.GREPANEASVEYLQEVAR.I | | 1009.501 | 2 | 2017.995 | 2017.994 | 0 | 0.6 | 4214 | 416.7 | 416.7 | 416.7 | 7.52 |
| 831 | R.GREPANEASVEYLQEVAR.I | | 1009.500 | 2 | 2017.993 | 2017.994 | 0 | −0.5 | 4214 | 386.6 | 386.6 | 386.6 | 7.11 |
| 831 | R.GREPANEASVEYLQEVAR.I | | 673.336 | 3 | 2017.995 | 2017.994 | 0 | 0.4 | 4214 | 342.0 | 342.0 | 342.0 | 5.89 |
| 831 | R.GREPANEASVEYLQEVAR.I | | 673.336 | 3 | 2017.994 | 2017.994 | 0 | 0.2 | 4214 | 339.0 | 339.0 | 339.0 | 5.89 |
| 1696 | R.IRLC[+57.021]LDR.A | C[+57] | 473.268 | 2 | 945.530 | 945.530 | 0 | −0.4 | 4232 | 381.3 | 381.3 | 381.3 | 5.85 |
| 1624 | R.AADFLSEPEGGPEM[+15.995]AKEK.Q | M[+16] | 641.300 | 3 | 1921.885 | 1921.885 | 0 0 | 0.2 | 4239 | 650.9 | 650.9 | 650.9 | 11.70 |
| 1232 | R.AADFLSEPEGGPEMAKEK.Q | | 635.968 | 3 | 1905.891 | 1905.890 | 0 | 0.4 | 4239 | 609.0 | 609.0 | 609.0 | 10.70 |
| 929 | R.AADFLSEPEGGPEMAK.E | | 824.880 | 2 | 1648.752 | 1648.752 | 0 | −0.3 | 4239 | 614.2 | 614.2 | 614.2 | 10.13 |
| 929 | R.AADFLSEPEGGPEMAK.E | | 824.880 | 2 | 1648.753 | 1648.752 | 0 | 0.1 | 4239 | 590.6 | 590.6 | 590.6 | 9.72 |
| 1232 | R.AADFLSEPEGGPEMAKEK.Q | | 635.969 | 3 | 1905.892 | 1905.890 | 0 | 0.9 | 4239 | 594.7 | 594.7 | 594.7 | 9.70 |
| 929 | R.AADFLSEPEGGPEMAK.E | | 824.880 | 2 | 1648.753 | 1648.752 | 0 | 0.6 | 4239 | 512.1 | 512.1 | 512.1 | 8.80 |
| 1618 | R.AADFLSEPEGGPEM[+15.995]AK.E | M[+16] | 832.878 | 2 | 1664.748 | 1664.747 | 0 | 0.3 | 4239 | 498.2 | 498.2 | 498.2 | 8.80 |
| 1232 | R.AADFLSEPEGGPEMAKEK.Q | | 953.449 | 2 | 1905.891 | 1905.890 | 0 | 0.3 | 4239 | 516.2 | 516.2 | 516.2 | 8.79 |
| 929 | R.AADFLSEPEGGPEMAK.E | | 824.880 | 2 | 1648.753 | 1648.752 | 0 | 0.3 | 4239 | 455.2 | 455.2 | 455.2 | 8.65 |
| 929 | R.AADFLSEPEGGPEMAK.E | | 550.256 | 3 | 1648.752 | 1648.752 | 0 | −0.1 | 4239 | 536.6 | 536.6 | 536.6 | 8.47 |
| 1624 | R.AADFLSEPEGGPEM[+15.995]AKEK.Q | M[+16] | 961.445 | 2 | 1921.883 | 1921.885 | 0 | 0.8 | 4239 | 540.3 | 540.3 | 540.3 | 8.39 |
| 929 | R.AADFLSEPEGGPEMAK.E | | 824.880 | 2 | 1648.753 | 1648.752 | 0 | 0.6 | 4239 | 507.7 | 507.7 | 507.7 | 8.21 |
| 1232 | R.AADFLSEPEGGPEMAKEK.Q | | 477.228 | 4 | 1905.889 | 1905.890 | 0 | −0.5 | 4239 | 536.7 | 536.7 | 536.7 | 7.87 |
| 1036 | R.GMEFVQGLSKPGRPHQWVFPK.D | | 809.092 | 3 | 2425.261 | 2425.260 | 0 | 0.4 | 4288 | 649.6 | 649.6 | 649.6 | 9.56 |
| 1036 | R.GMEFVQGLSKPGRPHQWVFPK.D | | 607.071 | 4 | 2425.260 | 2425.260 | 0 | 0.1 | 4288 | 495.2 | 495.2 | 495.2 | 9.07 |
| 1036 | R.GMEFVQGLSKPGRPHQWVFPK.D | | 607.070 | 4 | 2425.260 | 2425.260 | 0 | 0.0 | 4288 | 398.9 | 398.9 | 398.9 | 8.56 |
| 1036 | R.GMEFVQGLSKPGRPHQWVFPK.D | | 607.070 | 4 | 2425.259 | 2425.260 | 0 | 0.2 | 4288 | 370.6 | 370.6 | 370.6 | 7.97 |
| 1036 | R.GMEFVQGLSKPGRPHQWVFPK.D | | 809.426 | 3 | 2426.263 | 2425.260 | 1 | −0.1 | 4288 | 552.1 | 552.1 | 552.1 | 7.83 |
| 1551 | R.GM[+15.995]EFVQGLSKPGRPHQWVFPK.D | M[+16] | 489.057 | 5 | 2441.256 | 2441.255 | 0 | 0.3 | 4288 | 378.4 | 361.5 | 361.5 | 7.68 |
| 1551 | R.GM[+15.995]EFVQGLSKPGRPHQWVFPK.D | M[+16] | 814.423 | 3 | 2441.255 | 2441.255 | 0 | 0.3 | 4288 | 511.4 | 511.4 | 511.4 | 7.65 |
| 1036 | R.GMEFVQGLSKPGRPHQWVFPK.D | | 809.091 | 3 | 2425.257 | 2425.260 | 0 | 1.0 | 4288 | 547.1 | 547.1 | 547.1 | 7.24 |
| 1036 | R.GMEFVQGLSKPGRPHQWVFPK.D | | 485.858 | 5 | 2425.261 | 2425.260 | 0 | 0.5 | 4288 | 345.9 | 345.9 | 345.9 | 7.24 |
| 1551 | R.GM[+15.995]EFVQGLSKPGRPHQWVFPK.D | M[+16] | 489.057 | 5 | 2441.255 | 2441.255 | 0 | 0.0 | 4288 | 338.5 | 338.5 | 338.5 | 6.65 |
| 1036 | R.GMEFVQGLSKPGRPHQWVFPK.D | | 486.054 | 5 | 2426.242 | 2425.260 | 1 | −8.5 | 4288 | 334.7 | 334.7 | 334.7 | 4.60 |
| 1058 | K.DVVKQQGLRQDHPGQMDR.Y | | 703.021 | 3 | 2107.047 | 2107.046 | 0 | 0.4 | 4309 | 600.9 | 600.9 | 600.9 | 8.94 |
| 1058 | K.DVVKQQGLRQDHPGQMDR.Y | | 527.517 | 4 | 2107.047 | 2107.046 | 0 | 0.6 | 4309 | 371.0 | 371.0 | 371.0 | 7.06 |
| 1058 | K.DVVKQQGLRQDHPGQMDR.Y | | 422.215 | 5 | 2107.048 | 2107.046 | 0 | 0.7 | 4309 | 351.0 | 351.0 | 351.0 | 5.73 |
| 1527 | K.DVVKQQGLRQDHPGQM[+15.995]DR.Y | M[+16] | 531.516 | 4 | 2123.042 | 2123.041 | 0 | 0.4 | 4309 | 324.5 | 324.5 | 324.5 | 5.55 |
| 1527 | K.DVVKQQGLRQDHPGQM[+15.995]DR.Y | M[+16] | 531.516 | 4 | 2123.041 | 2123.041 | 0 | 0.1 | 4309 | 317.5 | 291.9 | 291.9 | 5.26 |
| 1527 | K.DVVKQQGLRQDHPGQM[+15.995]DR.Y | M[+16] | 531.516 | 4 | 2123.042 | 2123.041 | 0 | 0.3 | 4309 | 310.7 | 310.7 | 310.7 | 5.26 |
| 1880 | K.QQGLRQDHPGQMDRYLVYGDEYK.A | | 699.834 | 4 | 2796.316 | 2796.316 | 0 | 0.1 | 4313 | 437.1 | 437.1 | 437.1 | 7.17 |
| 1880 | K.QQGLRQDHPGQMDRYLVYGDEYK.A | | 560.069 | 5 | 2796.315 | 2796.316 | 0 | −0.2 | 4313 | 393.2 | 393.2 | 393.2 | 7.06 |
| 1880 | K.QQGLRQDHPGQMDRYLVYGDEYK.A | | 932.777 | 3 | 2796.317 | 2796.316 | 0 | 0.2 | 4313 | 480.2 | 480.2 | 480.2 | 7.02 |
| 1880 | K.QQGLRQDHPGQMDRYLVYGDEYK.A | | 560.069 | 5 | 2796.316 | 2796.316 | 0 | −0.1 | 4313 | 326.0 | 326.0 | 326.0 | 5.73 |
| 1544 | K.QQGLRQDHPGQM[+15.995]DRYLVYGDEYK.A | M[+16] | 704.084 | 4 | 2813.316 | 2812.311 | 1 | 0.6 | 4313 | 311.4 | 311.4 | 311.4 | 5.26 |
| 1544 | K.QQGLRQDHPGQM[+15.995]DRYLVYGDEYK.A | M[+16] | 563.268 | 5 | 2812.310 | 2812.311 | 0 | −0.2 | 4313 | 300.3 | 300.3 | 300.3 | 5.07 |
| 1880 | K.QQGLRQDHPGQMDRYLVYGDEYK.A | | 699.834 | 4 | 2796.315 | 2796.316 | 0 | −0.2 | 4313 | 332.9 | 332.9 | 332.9 | 4.96 |
| 1880 | K.QQGLRQDHPGQMDRYLVYGDEYK.A | | 932.778 | 3 | 2796.319 | 2796.316 | 0 | 1.0 | 4313 | 314.5 | 314.5 | 314.5 | 3.65 |
| 816 | R.QDHPGQMDRYLVYGDEYK.A | | 738.669 | 3 | 2213.993 | 2213.992 | 0 | 0.5 | 4318 | 388.7 | 388.7 | 388.7 | 7.22 |
| 816 | R.QDHPGQMDRYLVYGDEYK.A | | 554.253 | 4 | 2213.991 | 2213.992 | 0 | 0.6 | 4318 | 423.7 | 423.7 | 423.7 | 6.94 |
| 844 | R.YLVYGDEYKALR.D | | 745.388 | 2 | 1489.769 | 1489.769 | 0 | 0.2 | 4327 | 603.8 | 603.8 | 603.8 | 10.44 |
| 970 | R.YLVYGDEYKALRDAVAK.A | | 658.683 | 3 | 1974.034 | 1974.033 | 0 | 0.3 | 4327 | 416.7 | 416.7 | 416.7 | 7.47 |
| 970 | R.YLVYGDEYKALRDAVAK.A | | 494.264 | 4 | 1974.033 | 1974.033 | 0 | 0.2 | 4327 | 412.2 | 412.2 | 412.2 | 7.47 |
| 970 | R.YLVYGDEYKALRDAVAK.A | | 658.683 | 3 | 1974.033 | 1974.033 | 0 | 0.0 | 4327 | 441.7 | 441.7 | 441.7 | 7.40 |
| 1785 | R.YLVYGDEYK.A | | 575.277 | 2 | 1149.546 | 1149.546 | 0 | 0.1 | 4327 | 438.0 | 438.0 | 438.0 | 7.35 |
| 970 | R.YLVYGDEYKALRDAVAK.A | | 494.264 | 4 | 1974.033 | 1974.033 | 0 | 0.2 | 4327 | 383.3 | 383.3 | 383.3 | 7.35 |
| 970 | R.YLVYGDEYKALRDAVAK.A | | 494.264 | 4 | 1974.035 | 1974.033 | 0 | 0.8 | 4327 | 364.9 | 364.9 | 364.9 | 7.35 |
| 1785 | R.YLVYGDEYK.A | | 575.277 | 2 | 1149.546 | 1149.546 | 0 | 0.5 | 4327 | 478.4 | 478.4 | 478.4 | 7.18 |
| 1785 | R.YLVYGDEYK.A | | 575.277 | 2 | 1149.546 | 1149.546 | 0 | −0.5 | 4327 | 455.2 | 455.2 | 455.2 | 7.18 |
| 970 | R.YLVYGDEYKALRDAVAK.A | | 658.683 | 3 | 1974.033 | 1974.033 | 0 | 0.0 | 4327 | 369.4 | 369.4 | 369.4 | 7.06 |
| 970 | R.YLVYGDEYKALRDAVAK.A | | 987.521 | 2 | 1974.034 | 1974.033 | 0 | 0.4 | 4327 | 463.4 | 463.4 | 463.4 | 6.87 |
| 970 | R.YLVYGDEYKALRDAVAK.A | | 494.264 | 4 | 1974.033 | 1974.033 | 0 | 0.0 | 4327 | 321.1 | 321.1 | 321.1 | 5.55 |
| 1785 | R.YLVYGDEYK.A | | 575.277 | 2 | 1149.547 | 1149.546 | 0 | 0.5 | 4327 | 302.8 | 302.8 | 302.8 | 5.14 |
| 1785 | R.YLVYGDEYK.A | | 575.277 | 2 | 1149.547 | 1149.546 | 0 | 0.4 | 4327 | 316.4 | 316.4 | 316.4 | 5.10 |
| 1726 | K.AVLEC[+57.021]KPLGIK.T | C[+57] | 409.909 | 3 | 1227.713 | 1227.713 | 0 | 0.1 | 4344 | 572.8 | 572.8 | 572.8 | 9.72 |
| 1726 | K.AVLEC[+57.021]KPLGIK.T | C[+57] | 614.360 | 2 | 1227.713 | 1227.713 | 0 | −0.1 | 4344 | 595.1 | 595.1 | 595.1 | 9.12 |
| 1194 | K.TPQSQQSAYFLLTLFR.E | | 950.503 | 2 | 1899.999 | 1899.996 | 0 | 1.2 | 4362 | 497.0 | 497.0 | 497.0 | 8.80 |
| 1194 | K.TPQSQQSAYFLLTLFR.E | | 634.004 | 3 | 1899.998 | 1899.996 | 0 | 0.9 | 4362 | 364.3 | 364.3 | 364.3 | 7.19 |
| 1878 | R.EVAILYR.S | | 432.253 | 2 | 863.499 | 863.499 | 0 | 0.1 | 4378 | 456.4 | 456.4 | 456.4 | 8.05 |
| 1878 | R.EVAILYR.S | | 432.253 | 2 | 863.499 | 863.499 | 0 | 0.3 | 4378 | 423.0 | 423.0 | 423.0 | 7.35 |
| 1878 | R.EVAILYR.S | | 432.253 | 2 | 863.499 | 863.499 | 0 | 0.1 | 4378 | 344.0 | 344.0 | 344.0 | 5.91 |
| 1685 | R.SHNASLHPTPEQC[+57.021]EAVSK.F | C[+57] | 664.646 | 3 | 1991.924 | 1991.924 | 0 | 0.1 | 4385 | 758.2 | 758.2 | 758.2 | 13.98 |
| 1685 | R.SHNASLHPTPEQC[+57.021]EAVSK.F | C[+57] | 664.646 | 3 | 1991.924 | 1991.924 | 0 | 0.1 | 4385 | 746.9 | 746.9 | 746.9 | 12.98 |
| 1685 | R.SHNASLHPTPEQC[+57.021]EAVSK.F | C[+57] | 498.736 | 4 | 1991.924 | 1991.924 | 0 | −0.1 | 4385 | 478.0 | 478.0 | 478.0 | 8.91 |
| 1759 | K.ILSPPDISR.F | | 499.287 | 2 | 997.568 | 997.568 | 0 | −0.1 | 4409 | 373.4 | 373.4 | 373.4 | 7.23 |
| 1759 | K.ILSPPDISR.F | | 499.287 | 2 | 997.568 | 997.568 | 0 | −0.1 | 4409 | 370.1 | 370.1 | 370.1 | 7.23 |
| 1782 | K.ILSPPDISRFATSLVDNSVPLLR.A | | 837.471 | 3 | 2510.398 | 2510.398 | 0 | 0.0 | 4409 | 395.2 | 395.2 | 395.2 | 7.22 |
| 1759 | K.ILSPPDISR.F | | 499.287 | 2 | 997.568 | 997.568 | 0 | −0.1 | 4409 | 397.8 | 397.8 | 397.8 | 7.03 |
| 1759 | K.ILSPPDISR.F | | 499.288 | 2 | 997.568 | 997.568 | 0 | 0.0 | 4409 | 373.7 | 373.7 | 373.7 | 7.03 |
| 1759 | K.ILSPPDISR.F | | 499.287 | 2 | 997.567 | 997.568 | 0 | −0.3 | 4409 | 380.6 | 380.6 | 380.6 | 6.45 |
| 1759 | K.ILSPPDISR.F | | 499.287 | 2 | 997.567 | 997.568 | 0 | −0.2 | 4409 | 340.3 | 340.3 | 340.3 | 5.13 |
| 1759 | K.ILSPPDISR.F | | 499.287 | 2 | 997.567 | 997.568 | 0 | 0.4 | 4409 | 334.3 | 334.3 | 334.3 | 5.13 |
| 887 | R.FATSLVDNSVPLLR.A | | 766.427 | 2 | 1531.847 | 1531.848 | 0 | −0.3 | 4418 | 626.0 | 626.0 | 626.0 | 10.13 |
| 887 | R.FATSLVDNSVPLLR.A | | 766.428 | 2 | 1531.848 | 1531.848 | 0 | 0.1 | 4418 | 599.9 | 599.9 | 599.9 | 9.72 |
| 1485 | R.FATSLVDN[+0.984]SVPLLR.A | N[+1] | 766.920 | 2 | 1532.832 | 1532.832 | 0 | 0.3 | 4418 | 596.8 | 596.8 | 596.8 | 9.72 |
| 887 | R.FATSLVDNSVPLLR.A | | 766.427 | 2 | 1531.848 | 1531.848 | 0 | −0.3 | 4418 | 561.6 | 561.6 | 561.6 | 9.13 |
| 1485 | R.FATSLVDN[+0.984]SVPLLR.A | N[+1] | 766.920 | 2 | 1532.832 | 1532.832 | 0 | 0.3 | 4418 | 548.6 | 548.6 | 548.6 | 8.98 |
| 1485 | R.FATSLVDN[+0.984]SVPLLR.A | N[+1] | 766.920 | 2 | 1532.832 | 1532.832 | 0 | 0.4 | 4418 | 531.7 | 531.7 | 531.7 | 8.98 |
| 1485 | R.FATSLVDN[+0.984]SVPLLR.A | N[+1] | 766.920 | 2 | 1532.832 | 1532.832 | 0 | 0.2 | 4418 | 488.6 | 488.6 | 488.6 | 8.80 |
| 1485 | R.FATSLVDN[+0.984]SVPLLR.A | N[+1] | 766.920 | 2 | 1532.832 | 1532.832 | 0 | 0.2 | 4418 | 464.3 | 464.3 | 464.3 | 8.05 |
| 887 | R.FATSLVDNSVPLLR.A | | 766.428 | 2 | 1531.848 | 1531.848 | 0 | 0.2 | 4418 | 415.0 | 415.0 | 415.0 | 7.83 |
| 887 | R.FATSLVDNSVPLLR.A | | 766.427 | 2 | 1531.848 | 1531.848 | 0 | −0.3 | 4418 | 550.1 | 550.1 | 550.1 | 7.80 |
| 887 | R.FATSLVDNSVPLLR.A | | 766.428 | 2 | 1531.848 | 1531.848 | 0 | 0.3 | 4418 | 388.3 | 388.3 | 388.3 | 7.42 |
| 1485 | R.FATSLVDN[+0.984]SVPLLR.A | N[+1] | 511.616 | 3 | 1532.832 | 1532.832 | 0 | 0.2 | 4418 | 426.0 | 426.0 | 426.0 | 7.02 |
| 887 | R.FATSLVDNSVPLLR.A | | 511.288 | 3 | 1531.848 | 1531.848 | 0 | 0.1 | 4418 | 420.2 | 420.2 | 420.2 | 7.02 |
| 1485 | R.FATSLVDN[+0.984]SVPLLR.A | N[+1] | 511.615 | 3 | 1532.832 | 1532.832 | 0 | −0.1 | 4418 | 361.0 | 361.0 | 361.0 | 6.90 |
| 887 | R.FATSLVDNSVPLLR.A | | 511.287 | 3 | 1531.847 | 1531.848 | 0 | 0.8 | 4418 | 428.3 | 428.3 | 428.3 | 6.72 |
| 1098 | K.NLAFSPATMAHAFLPTMPEDLLAQAR.R | | 938.476 | 3 | 2813.413 | 2813.411 | 0 | 0.7 | 4467 | 857.6 | 857.6 | 857.6 | 15.95 |
| 941 | K.NLAFSPATMAHAFLPTMPEDLLAQARR.W | | 743.134 | 4 | 2969.515 | 2969.512 | 0 | 0.9 | 4467 | 601.6 | 601.6 | 601.6 | 10.70 |
| 1098 | K.NLAFSPATMAHAFLPTMPEDLLAQAR.R | | 1407.214 | 2 | 2813.420 | 2813.411 | 0 | 3.0 | 4467 | 668.8 | 570.7 | 570.7 | 10.57 |
| 1098 | K.NLAFSPATMAHAFLPTMPEDLLAQAR.R | | 938.474 | 3 | 2813.408 | 2813.411 | 0 | −1.2 | 4467 | 581.4 | 524.1 | 524.1 | 9.39 |
| 1098 | K.NLAFSPATMAHAFLPTMPEDLLAQAR.R | | 704.109 | 4 | 2813.414 | 2813.411 | 0 | 1.0 | 4467 | 552.8 | 552.8 | 552.8 | 8.73 |
| 1628 | K.NLAFSPATMAHAFLPTM[+15.995]PEDLLAQAR.R | M[+16] | 943.808 | 3 | 2829.411 | 2829.406 | 0 | 1.5 | 4467 | 553.9 | 511.9 | 5.0 | 8.65 |
| 1639 | K.NLAFSPATM[+15.995]AHAFLPTMPEDLLAQAR.R | M[+16] | 943.807 | 3 | 2829.405 | 2829.406 | 0 | 0.3 | 4467 | 547.1 | 547.1 | 6.7 | 8.06 |
| 1098 | K.NLAFSPATMAHAFLPTMPEDLLAQAR.R | | 938.475 | 3 | 2813.411 | 2813.411 | 0 | −0.1 | 4467 | 429.8 | 415.2 | 415.2 | 7.80 |
| 941 | K.NLAFSPATMAHAFLPTMPEDLLAQARR.W | | 743.134 | 4 | 2969.516 | 2969.512 | 0 | 1.1 | 4467 | 450.3 | 409.2 | 409.2 | 7.56 |
| 1303 | R.DGFHLVKDKADR.T | | 467.581 | 3 | 1400.728 | 1400.728 | 0 | −0.2 | 4541 | 669.2 | 574.4 | 574.4 | 9.09 |
| 1303 | R.DGFHLVKDKADR.T | | 700.868 | 2 | 1400.728 | 1400.728 | 0 | 0.2 | 4541 | 504.4 | 504.4 | 504.4 | 6.17 |
| 1303 | R.DGFHLVKDKADR.T | | 467.581 | 3 | 1400.728 | 1400.728 | 0 | −0.1 | 4541 | 369.5 | 369.5 | 369.5 | 5.19 |
| 2683 | K.ADRTQTGHVLGNPQRR.D | | 452.243 | 4 | 1805.949 | 1805.948 | 0 | 0.7 | 4550 | 585.6 | 585.6 | 585.6 | 9.09 |
| 2009 | R.TQTGHVLGNPQRR.D | | 488.599 | 3 | 1463.783 | 1463.783 | 0 | 0.3 | 4553 | 375.6 | 375.6 | 375.6 | 6.96 |
| 886 | K.GFLQQHILKDLEQLAK.M | | 627.692 | 3 | 1881.060 | 1881.059 | 0 | 0.6 | 4614 | 634.3 | 634.3 | 634.3 | 10.44 |
| 886 | K.GFLQQHILKDLEQLAK.M | | 471.020 | 4 | 1881.059 | 1881.059 | 0 | 0.1 | 4614 | 485.9 | 485.9 | 485.9 | 8.53 |
| 886 | K.GFLQQHILKDLEQLAK.M | | 627.692 | 3 | 1881.060 | 1881.059 | 0 | 0.5 | 4614 | 484.2 | 484.2 | 484.2 | 8.53 |
| 886 | K.GFLQQHILKDLEQLAK.M | | 941.033 | 2 | 1881.060 | 1881.059 | 0 | 0.1 | 4614 | 469.4 | 469.4 | 469.4 | 6.96 |
| 1043 | K.MLGHSADETIGVVHLVLR.R | | 649.688 | 3 | 1947.050 | 1947.048 | 0 | 0.8 | 4630 | 802.1 | 802.1 | 802.1 | 14.95 |
| 1043 | K.MLGHSADETIGVVHLVLR.R | | 487.517 | 4 | 1947.048 | 1947.048 | 0 | −0.3 | 4630 | 637.2 | 637.2 | 637.2 | 10.39 |
| 819 | K.MLGHSADETIGVVHLVLRR.L | | 526.543 | 4 | 2103.149 | 2103.149 | 0 | −0.2 | 4630 | 584.1 | 584.1 | 584.1 | 9.12 |
| 1625 | K.M[+15.995]LGHSADETIGVVHLVLRR.L | M[+16] | 424.635 | 5 | 2119.145 | 2119.144 | 0 | 0.5 | 4630 | 516.6 | 516.6 | 516.6 | 8.79 |
| 819 | K.MLGHSADETIGVVHLVLRR.L | | 421.436 | 5 | 2103.149 | 2103.149 | 0 | 0.0 | 4630 | 506.8 | 506.8 | 506.8 | 8.79 |
| 1043 | K.MLGHSADETIGVVHLVLR.R | | 487.517 | 4 | 1947.048 | 1947.048 | 0 | −0.3 | 4630 | 363.6 | 363.6 | 363.6 | 7.09 |
| 1043 | K.MLGHSADETIGVVHLVLR.R | | 649.688 | 3 | 1947.048 | 1947.048 | 0 | −0.1 | 4630 | 339.5 | 339.5 | 339.5 | 6.17 |
| 924 | R.RLLQEQHQLSSR.R | | 498.943 | 3 | 1494.814 | 1494.814 | 0 | 0.0 | 4648 | 476.5 | 476.5 | 476.5 | 8.05 |
| 924 | R.RLLQEQHQLSSR.R | | 498.943 | 3 | 1494.814 | 1494.814 | 0 | 0.2 | 4648 | 444.8 | 444.8 | 444.8 | 8.05 |
| 924 | R.RLLQEQHQLSSR.R | | 498.943 | 3 | 1494.814 | 1494.814 | 0 | 0.1 | 4648 | 419.8 | 419.8 | 419.8 | 7.35 |
| 1824 | R.LLQEQHQLSSR.R | | 669.860 | 2 | 1338.713 | 1338.712 | 0 | 0.0 | 4649 | 636.7 | 636.7 | 636.7 | 10.72 |
| 1824 | R.LLQEQHQLSSR.R | | 446.909 | 3 | 1338.713 | 1338.712 | 0 | 0.4 | 4649 | 428.7 | 428.7 | 428.7 | 7.53 |
| 1824 | R.LLQEQHQLSSR.R | | 446.909 | 3 | 1338.712 | 1338.712 | 0 | −0.1 | 4649 | 408.8 | 408.8 | 408.8 | 7.53 |
| 1824 | R.LLQEQHQLSSR.R | | 446.909 | 3 | 1338.712 | 1338.712 | 0 | −0.3 | 4649 | 449.0 | 449.0 | 449.0 | 7.18 |
| 1824 | R.LLQEQHQLSSR.R | | 446.910 | 3 | 1338.714 | 1338.712 | 0 | 1.2 | 4649 | 316.8 | 316.8 | 316.8 | 5.43 |
| 1762 | R.LLQEQHQLSSRR.L | | 498.943 | 3 | 1494.813 | 1494.814 | 0 | −0.3 | 4649 | 352.2 | 276.8 | 276.8 | 4.75 |
| 906 | R.RLLNFDTELSTK.E | | 718.891 | 2 | 1436.774 | 1436.774 | 0 | 0.0 | 4660 | 551.9 | 551.9 | 551.9 | 8.39 |
| 906 | R.RLLNFDTELSTK.E | | 479.596 | 3 | 1436.774 | 1436.774 | 0 | 0.1 | 4660 | 498.5 | 498.5 | 498.5 | 8.21 |
| 906 | R.RLLNFDTELSTK.E | | 479.596 | 3 | 1436.774 | 1436.774 | 0 | 0.1 | 4660 | 469.1 | 469.1 | 469.1 | 8.05 |
| 1111 | R.RLLNFDTELSTKEMR.N | | 463.995 | 4 | 1852.957 | 1852.959 | 0 | 0.6 | 4660 | 517.8 | 517.8 | 517.8 | 7.95 |
| 906 | R.RLLNFDTELSTK.E | | 479.596 | 3 | 1436.774 | 1436.774 | 0 | 0.2 | 4660 | 492.8 | 492.8 | 492.8 | 7.62 |
| 906 | R.RLLNFDTELSTK.E | | 718.891 | 2 | 1436.775 | 1436.774 | 0 | 0.1 | 4660 | 426.5 | 426.5 | 426.5 | 7.53 |
| 906 | R.RLLNFDTELSTK.E | | 479.596 | 3 | 1436.774 | 1436.774 | 0 | 0.1 | 4660 | 417.1 | 417.1 | 417.1 | 7.53 |
| 906 | R.RLLNFDTELSTK.E | | 479.596 | 3 | 1436.774 | 1436.774 | 0 | −0.1 | 4660 | 413.3 | 413.3 | 413.3 | 7.53 |
| 906 | R.RLLNFDTELSTK.E | | 479.596 | 3 | 1436.775 | 1436.774 | 0 | 0.1 | 4660 | 383.4 | 383.4 | 383.4 | 7.23 |
| 1484 | R.RLLN[+0.984]FDTELSTK.E | N[+1] | 479.925 | 3 | 1437.759 | 1437.758 | 0 | 0.3 | 4660 | 368.0 | 222.6 | 222.6 | 5.55 |
| 906 | R.RLLNFDTELSTK.E | | 479.596 | 3 | 1436.774 | 1436.774 | 0 | 0.4 | 4660 | 347.0 | 347.0 | 347.0 | 5.51 |
| 873 | R.LLNFDTELSTKEMR.N | | 566.291 | 3 | 1696.857 | 1696.857 | 0 | −0.1 | 4661 | 496.6 | 496.6 | 496.6 | 8.53 |
| 821 | R.LLNFDTELSTK.E | | 640.840 | 2 | 1280.673 | 1280.673 | 0 | 0.1 | 4661 | 486.6 | 457.5 | 457.5 | 8.21 |
| 1533 | R.LLNFDTELSTKEM[+15.995]R.N | M[+16] | 571.622 | 3 | 1712.852 | 1712.852 | 0 | 0.0 | 4661 | 532.4 | 532.4 | 532.4 | 8.12 |
| 821 | R.LLNFDTELSTK.E | | 640.841 | 2 | 1280.674 | 1280.673 | 0 | 0.5 | 4661 | 483.5 | 483.5 | 483.5 | 7.92 |
| 821 | R.LLNFDTELSTK.E | | 640.841 | 2 | 1280.674 | 1280.673 | 0 | 0.7 | 4661 | 462.2 | 462.2 | 462.2 | 7.76 |
| 821 | R.LLNFDTELSTK.E | | 640.840 | 2 | 1280.674 | 1280.673 | 0 | 0.2 | 4661 | 442.1 | 394.3 | 394.3 | 7.76 |
| 821 | R.LLNFDTELSTK.E | | 640.840 | 2 | 1280.673 | 1280.673 | 0 | 0.1 | 4661 | 437.1 | 371.8 | 371.8 | 7.53 |
| 821 | R.LLNFDTELSTK.E | | 640.841 | 2 | 1280.674 | 1280.673 | 0 | 0.3 | 4661 | 424.3 | 395.5 | 395.5 | 7.53 |
| 821 | R.LLNFDTELSTK.E | | 640.841 | 2 | 1280.674 | 1280.673 | 0 | 0.5 | 4661 | 451.6 | 353.8 | 353.8 | 6.53 |
| 821 | R.LLNFDTELSTK.E | | 640.840 | 2 | 1280.673 | 1280.673 | 0 | 0.0 | 4661 | 352.8 | 352.8 | 352.8 | 5.91 |
| 821 | R.LLNFDTELSTK.E | | 640.840 | 2 | 1280.673 | 1280.673 | 0 | −0.1 | 4661 | 401.0 | 253.3 | 253.3 | 5.62 |
| 821 | R.LLNFDTELSTK.E | | 427.563 | 3 | 1280.674 | 1280.673 | 0 | 0.2 | 4661 | 326.5 | 258.4 | 258.4 | 4.90 |
| 1321 | R.NNWEKEIAAVISPELEHLDK.T | | 779.069 | 3 | 2335.193 | 2335.193 | 0 | −0.1 | 4675 | 650.1 | 650.1 | 650.1 | 11.70 |
| 1321 | R.NNWEKEIAAVISPELEHLDK.T | | 779.069 | 3 | 2335.193 | 2335.193 | 0 | 0.0 | 4675 | 613.3 | 613.3 | 613.3 | 10.70 |
| 1014 | R.NNWEKEIAAVISPELEHLDKTLPTMNNLISQDK.R | | 758.992 | 5 | 3790.932 | 3790.932 | 0 | −0.1 | 4675 | 612.3 | 612.3 | 612.3 | 9.64 |
| 1321 | R.NNWEKEIAAVISPELEHLDK.T | | 779.070 | 3 | 2335.194 | 2335.193 | 0 | 0.5 | 4675 | 582.8 | 582.8 | 582.8 | 9.11 |
| 1498 | R.N[+0.984]NWEKEIAAVISPELEHLDK.T | N[+1] | 584.800 | 4 | 2336.179 | 2336.177 | 0 | 1.1 | 4675 | 518.3 | 518.3 | 0.0 | 8.79 |
| 1321 | R.NNWEKEIAAVISPELEHLDK.T | | 1168.101 | 2 | 2335.194 | 2335.193 | 0 | 0.6 | 4675 | 490.7 | 490.7 | 490.7 | 7.37 |
| 1498 | R.N[+0.984]NWEKEIAAVISPELEHLDK.T | N[+1] | 779.398 | 3 | 2336.180 | 2336.177 | 0 | 1.2 | 4675 | 408.7 | 408.7 | 0.0 | 7.33 |
| 1933 | K.EIAAVISPELEHLDK.T | | 555.302 | 3 | 1663.890 | 1663.890 | 0 | −0.2 | 4680 | 609.6 | 609.6 | 609.6 | 10.13 |
| 1055 | K.EIAAVISPELEHLDKTLPTMNNLISQDKR.I | | 1092.915 | 3 | 3276.730 | 3275.730 | 1 | −1.3 | 4680 | 728.8 | 728.8 | 298.6 | 9.64 |
| 933 | K.EIAAVISPELEHLDK.T | | 555.301 | 3 | 1663.889 | 1663.890 | 0 | −0.4 | 4680 | 571.8 | 571.8 | 571.8 | 9.13 |
| 1597 | K.EIAAVISPELEHLDKTLPTM[+15.995]NNLISQDKR.I | M[+16] | 659.151 | 5 | 3291.724 | 3291.725 | 0 | −0.5 | 4680 | 559.4 | 559.4 | 559.4 | 7.33 |
| 1055 | K.EIAAVISPELEHLDKTLPTMNNLISQDKR.I | | 819.688 | 4 | 3275.729 | 3275.730 | 0 | −0.5 | 4680 | 553.0 | 553.0 | 553.0 | 7.33 |
| 1055 | K.EIAAVISPELEHLDKTLPTMNNLISQDKR.I | | 819.688 | 4 | 3275.729 | 3275.730 | 0 | 0.4 | 4680 | 511.8 | 511.8 | 511.8 | 7.15 |
| 1597 | K.EIAAVISPELEHLDKTLPTM[+15.995]NNLISQDKR.I | M[+16] | 659.151 | 5 | 3291.726 | 3291.725 | 0 | 0.3 | 4680 | 434.3 | 434.3 | 434.3 | 6.75 |
| 1055 | K.EIAAVISPELEHLDKTLPTMNNLISQDKR.I | | 1093.249 | 3 | 3277.733 | 3275.730 | 2 | −1.1 | 4680 | 561.4 | 561.4 | 15.1 | 6.64 |
| 1597 | K.EIAAVISPELEHLDKTLPTM[+15.995]NNLISQDKR.I | M[+16] | 823.687 | 4 | 3291.726 | 3291.725 | 0 | 0.2 | 4680 | 415.0 | 415.0 | 415.0 | 6.46 |
| 1055 | K.EIAAVISPELEHLDKTLPTMNNLISQDKR.I | | 1092.581 | 3 | 3275.727 | 3275.730 | 0 | −1.1 | 4680 | 396.3 | 396.3 | 396.3 | 4.34 |
| 882 | K.TLPTMNNLISQDKR.I | | 815.933 | 2 | 1630.859 | 1630.858 | 0 | 0.6 | 4695 | 558.6 | 558.6 | 558.6 | 8.71 |
| 882 | K.TLPTMNNLISQDKR.I | | 544.291 | 3 | 1630.858 | 1630.858 | 0 | 0.1 | 4695 | 519.1 | 519.1 | 519.1 | 7.94 |
| 1574 | K.TLPTM[+15.995]NNLISQDKR.I | M[+16] | 549.622 | 3 | 1646.853 | 1646.853 | 0 | 0.2 | 4695 | 395.9 | 395.9 | 395.9 | 7.43 |
| 882 | K.TLPTMNNLISQDKR.I | | 544.291 | 3 | 1630.859 | 1630.858 | 0 | 0.4 | 4695 | 368.4 | 368.4 | 368.4 | 6.96 |
| 1574 | K.TLPTM[+15.995]NNLISQDKR.I | M[+16] | 549.622 | 3 | 1646.852 | 1646.853 | 0 | 0.5 | 4695 | 442.3 | 442.3 | 442.3 | 6.90 |
| 1574 | K.TLPTM[+15.995]NNLISQDKR.I | M[+16] | 549.622 | 3 | 1646.852 | 1646.853 | 0 | 0.8 | 4695 | 375.0 | 375.0 | 375.0 | 6.56 |
| 882 | K.TLPTMNNLISQDKR.I | | 544.291 | 3 | 1630.858 | 1630.858 | 0 | −0.2 | 4695 | 363.6 | 363.6 | 363.6 | 6.56 |
| 882 | K.TLPTMNNLISQDKR.I | | 544.291 | 3 | 1630.858 | 1630.858 | 0 | 0.1 | 4695 | 315.9 | 315.9 | 315.9 | 5.35 |
| 882 | K.TLPTMNNLISQDKR.I | | 544.291 | 3 | 1630.858 | 1630.858 | 0 | 0.2 | 4695 | 338.4 | 338.4 | 338.4 | 5.24 |
| 1481 | K.TLPTMN[+0.984]NLISQDKR.I | N[+1] | 544.619 | 3 | 1631.842 | 1631.842 | 0 | 0.2 | 4695 | 333.0 | 283.2 | 76.1 | 5.24 |
| 882 | K.TLPTMNNLISQDKR.I | | 544.291 | 3 | 1630.857 | 1630.858 | 0 | −0.4 | 4695 | 351.4 | 351.4 | 351.4 | 5.05 |
| 1838 | R.ISSNPVAK.I | | 408.234 | 2 | 815.461 | 815.462 | 0 | 1.9 | 4709 | 403.6 | 403.6 | 403.6 | 6.57 |
| 1838 | R.ISSNPVAK.I | | 408.234 | 2 | 815.461 | 815.462 | 0 | −1.7 | 4709 | 324.5 | 324.5 | 324.5 | 5.13 |
| 1172 | K.IIYGDPVTFLPHLPR.K | | 869.488 | 2 | 1737.968 | 1737.969 | 0 | −0.4 | 4717 | 694.0 | 694.0 | 694.0 | 11.13 |
| 846 | K.IIYGDPVTFLPHLPRK.S | | 467.272 | 4 | 1866.064 | 1866.064 | 0 | 0.3 | 4717 | 628.8 | 628.8 | 628.8 | 10.44 |
| 846 | K.IIYGDPVTFLPHLPRK.S | | 467.271 | 4 | 1866.064 | 1866.064 | 0 | 0.2 | 4717 | 621.5 | 621.5 | 621.5 | 10.44 |
| 1172 | K.IIYGDPVTFLPHLPR.K | | 579.994 | 3 | 1737.969 | 1737.969 | 0 | 0.0 | 4717 | 570.9 | 570.9 | 570.9 | 9.72 |
| 1172 | K.IIYGDPVTFLPHLPR.K | | 579.994 | 3 | 1737.968 | 1737.969 | 0 | −0.1 | 4717 | 492.7 | 492.7 | 492.7 | 8.80 |
| 1172 | K.IIYGDPVTFLPHLPR.K | | 579.994 | 3 | 1737.968 | 1737.969 | 0 | 0.2 | 4717 | 558.3 | 558.3 | 558.3 | 8.40 |
| 1172 | K.IIYGDPVTFLPHLPR.K | | 869.487 | 2 | 1737.967 | 1737.969 | 0 | −1.1 | 4717 | 528.7 | 528.7 | 528.7 | 8.40 |
| 846 | K.IIYGDPVTFLPHLPRK.S | | 622.693 | 3 | 1866.065 | 1866.064 | 0 | 0.6 | 4717 | 452.4 | 452.4 | 452.4 | 8.38 |
| 846 | K.IIYGDPVTFLPHLPRK.S | | 467.271 | 4 | 1866.064 | 1866.064 | 0 | 0.0 | 4717 | 437.4 | 437.4 | 437.4 | 8.15 |
| 1172 | K.IIYGDPVTFLPHLPR.K | | 435.248 | 4 | 1737.969 | 1737.969 | 0 | 0.4 | 4717 | 471.2 | 471.2 | 471.2 | 8.13 |
| 1172 | K.IIYGDPVTFLPHLPR.K | | 579.995 | 3 | 1737.969 | 1737.969 | 0 | 0.2 | 4717 | 381.9 | 381.9 | 381.9 | 7.71 |
| 1172 | K.IIYGDPVTFLPHLPR.K | | 580.329 | 3 | 1738.971 | 1737.969 | 1 | 0.4 | 4717 | 365.6 | 365.6 | 365.6 | 6.83 |
| 846 | K.IIYGDPVTFLPHLPRK.S | | 933.536 | 2 | 1866.065 | 1866.064 | 0 | 0.5 | 4717 | 434.6 | 434.6 | 434.6 | 6.73 |
| 2008 | R.KRITVEYLQHIVEQK.N | | 471.773 | 4 | 1884.070 | 1884.070 | 0 | 0.0 | 4745 | 510.3 | 457.8 | 457.8 | 7.94 |
| 2008 | R.KRITVEYLQHIVEQK.N | | 471.773 | 4 | 1884.070 | 1884.070 | 0 | 0.1 | 4745 | 342.7 | 342.7 | 342.7 | 5.82 |
| 2337 | K.RITVEYLQHIVEQK.N | | 439.750 | 4 | 1755.976 | 1755.975 | 0 | 0.6 | 4746 | 589.2 | 589.2 | 589.2 | 9.72 |
| 2337 | K.RITVEYLQHIVEQK.N | | 585.996 | 3 | 1755.975 | 1755.975 | 0 | −0.4 | 4746 | 454.0 | 454.0 | 454.0 | 8.06 |
| 1159 | R.ITVEYLQHIVEQK.N | | 800.441 | 2 | 1599.875 | 1599.874 | 0 | 0.4 | 4747 | 718.1 | 665.7 | 665.7 | 12.72 |
| 1159 | R.ITVEYLQHIVEQK.N | | 533.963 | 3 | 1599.874 | 1599.874 | 0 | −0.2 | 4747 | 664.6 | 664.6 | 664.6 | 11.72 |
| 1159 | R.ITVEYLQHIVEQK.N | | 800.441 | 2 | 1599.874 | 1599.874 | 0 | 0.0 | 4747 | 636.1 | 532.2 | 532.2 | 10.72 |
| 1159 | R.ITVEYLQHIVEQK.N | | 533.963 | 3 | 1599.874 | 1599.874 | 0 | 0.0 | 4747 | 621.6 | 621.6 | 621.6 | 10.72 |
| 1512 | R.ITVEYLQHIVEQKN[+0.984]GKER.V | N[+1] | 547.046 | 4 | 2185.161 | 2185.161 | 0 | 0.0 | 4747 | 595.2 | 595.2 | 505.5 | 9.36 |
| 1159 | R.ITVEYLQHIVEQK.N | | 800.441 | 2 | 1599.875 | 1599.874 | 0 | 0.4 | 4747 | 555.5 | 453.9 | 453.9 | 8.98 |
| 879 | R.VPILWHFLQKEAELR.L | | 470.520 | 4 | 1879.059 | 1879.059 | 0 | 0.2 | 4765 | 442.5 | 442.5 | 442.5 | 8.38 |
| 863 | R.VPILWHFLQK.E | | 640.880 | 2 | 1280.752 | 1280.751 | 0 | 0.4 | 4765 | 456.8 | 456.8 | 456.8 | 7.76 |
| 863 | R.VPILWHFLQK.E | | 427.589 | 3 | 1280.752 | 1280.751 | 0 | 0.2 | 4765 | 442.0 | 442.0 | 442.0 | 7.76 |
| 863 | R.VPILWHFLQK.E | | 427.589 | 3 | 1280.751 | 1280.751 | 0 | −0.1 | 4765 | 406.8 | 406.8 | 406.8 | 7.53 |
| 879 | R.VPILWHFLQKEAELR.L | | 470.770 | 4 | 1880.057 | 1879.059 | 1 | −2.8 | 4765 | 440.6 | 440.6 | 440.6 | 7.00 |
| 853 | K.FLPEILALQR.D | | 600.361 | 2 | 1199.715 | 1199.715 | 0 | 0.4 | 4783 | 439.9 | 439.9 | 439.9 | 7.35 |
| 853 | K.FLPEILALQR.D | | 600.361 | 2 | 1199.715 | 1199.715 | 0 | 0.2 | 4783 | 425.0 | 425.0 | 425.0 | 7.35 |
| 853 | K.FLPEILALQR.D | | 400.576 | 3 | 1199.715 | 1199.715 | 0 | 0.1 | 4783 | 441.5 | 441.5 | 441.5 | 7.05 |
| 853 | K.FLPEILALQR.D | | 600.361 | 2 | 1199.715 | 1199.715 | 0 | 0.0 | 4783 | 362.7 | 362.7 | 362.7 | 7.03 |
| 853 | K.FLPEILALQR.D | | 600.361 | 2 | 1199.715 | 1199.715 | 0 | 0.6 | 4783 | 313.3 | 313.3 | 313.3 | 5.14 |
| 1019 | R.DLVKQFQNVQQVEYSSIR.G | | 1091.069 | 2 | 2181.131 | 2181.130 | 0 | 0.5 | 4793 | 492.0 | 425.9 | 425.9 | 8.79 |
| 1019 | R.DLVKQFQNVQQVEYSSIR.G | | 727.715 | 3 | 2181.131 | 2181.130 | 0 | 0.3 | 4793 | 477.2 | 477.2 | 477.2 | 8.64 |
| 1019 | R.DLVKQFQNVQQVEYSSIR.G | | 1091.069 | 2 | 2181.131 | 2181.130 | 0 | 0.5 | 4793 | 381.7 | 381.7 | 381.7 | 8.29 |
| 1019 | R.DLVKQFQNVQQVEYSSIR.G | | 727.716 | 3 | 2181.132 | 2181.130 | 0 | 0.9 | 4793 | 458.7 | 458.7 | 458.7 | 8.04 |
| 1019 | R.DLVKQFQNVQQVEYSSIR.G | | 728.050 | 3 | 2182.136 | 2181.130 | 1 | 1.0 | 4793 | 418.5 | 418.5 | 159.7 | 7.52 |
| 1019 | R.DLVKQFQNVQQVEYSSIR.G | | 727.715 | 3 | 2181.131 | 2181.130 | 0 | 0.4 | 4793 | 406.9 | 406.9 | 406.9 | 7.52 |
| 818 | K.QFQNVQQVEYSSIR.G | | 863.431 | 2 | 1725.854 | 1725.856 | 0 | −0.7 | 4797 | 619.9 | 619.9 | 619.9 | 10.13 |
| 818 | K.QFQNVQQVEYSSIR.G | | 575.956 | 3 | 1725.855 | 1725.856 | 0 | −0.4 | 4797 | 495.3 | 495.3 | 495.3 | 7.70 |
| 925 | R.ITVFLSTWNK.L | | 604.837 | 2 | 1208.667 | 1208.667 | 0 | −0.3 | 4829 | 484.7 | 484.7 | 484.7 | 7.33 |
| 1720 | R.SLETNGEINLPKDYC[+57.021]STDLDLDTEFEILLPR.R | C[+57] | 1204.588 | 3 | 3611.748 | 3610.747 | 1 | −0.6 | 4842 | 759.8 | 696.8 | 308.4 | 12.41 |
| 1146 | R.SLETNGEINLPK.D | | 657.849 | 2 | 1314.690 | 1314.690 | 0 | 0.0 | 4842 | 581.5 | 581.5 | 581.5 | 9.72 |
| 1720 | R.SLETNGEINLPKDYC[+57.021]STDLDLDTEFEILLPR.R | C[+57] | 1204.249 | 3 | 3610.733 | 3610.747 | 0 | −3.7 | 4842 | 423.1 | 310.9 | 310.9 | 3.32 |
| 1715 | R.GLGLC[+57.021]ATALVSYLIR.L | C[+57] | 536.304 | 3 | 1606.899 | 1606.899 | 0 | 0.2 | 4875 | 508.9 | 508.9 | 508.9 | 8.29 |
| 1819 | R.LHNEIVYAVEKLSK.E | | 411.485 | 4 | 1642.916 | 1642.916 | 0 | 0.1 | 4890 | 489.4 | 489.4 | 489.4 | 8.53 |
| 1246 | R.LHNEIVYAVEK.L | | 438.907 | 3 | 1314.705 | 1314.705 | 0 | 0.3 | 4890 | 429.0 | 429.0 | 429.0 | 7.24 |
| 1246 | R.LHNEIVYAVEK.L | | 438.907 | 3 | 1314.705 | 1314.705 | 0 | −0.2 | 4890 | 374.6 | 374.6 | 374.6 | 7.23 |
| 1819 | R.LHNEIVYAVEKLSK.E | | 411.485 | 4 | 1642.916 | 1642.916 | 0 | 0.0 | 4890 | 340.5 | 340.5 | 340.5 | 5.63 |
| 1819 | R.LHNEIVYAVEKLSK.E | | 411.485 | 1 | 1642.917 | 1642.916 | 0 | 0.4 | 4890 | 306.9 | 306.9 | 306.9 | 5.16 |
| 4652 | R.DLTPLILSNC[+57.021]QYQVEEGRETVQEFDLEK.I | C[+57] | 1118.880 | 3 | 3354.626 | 3353.621 | 1 | 0.6 | 4928 | 394.2 | 394.2 | 14.0 | 7.02 |
| 900 | R.ETVQEFDLEK.I | | 619.301 | 2 | 1237.595 | 1237.595 | 0 | 0.0 | 4946 | 454.6 | 317.0 | 317.0 | 6.14 |
| 1004 | R.LSLKGIPTLVYR.H | | 453.950 | 3 | 1359.836 | 1359.836 | 0 | −0.1 | 4971 | 622.5 | 622.5 | 622.5 | 10.44 |
| 1912 | K.GIPTLVYR.H | | 459.774 | 2 | 918.541 | 918.541 | 0 | 0.4 | 4975 | 354.0 | 354.0 | 354.0 | 5.71 |
| 966 | R.HDWNYEHLFMDIK.N | | 583.268 | 3 | 1747.789 | 1747.790 | 0 | −0.5 | 4983 | 508.4 | 508.4 | 508.4 | 8.22 |
| 1611 | R.HDWNYEHLFM[+15.995]DIKNK.M | M[+16] | 502.236 | 1 | 2005.923 | 2005.923 | 0 | 0.3 | 4983 | 489.1 | 489.1 | 489.1 | 7.94 |
| 1565 | R.HDWNYEHLFM[+15.995]DIK.N | M[+16 | 441.702 | 4 | 1763.785 | 1763.785 | 0 | 0.0 | 4983 | 362.2 | 362.2 | 362.2 | 7.23 |
| 1565 | R.HDWNYEHLFM[+15.995]DIK.N | M[+16] | 588.600 | 3 | 1763.784 | 1763.785 | 0 | 0.2 | 4983 | 472.4 | 472.4 | 472.4 | 7.18 |
| 1565 | R.HDWNYEHLFM[+15.995]DIK.N | M[+16] | 588.600 | 3 | 1763.784 | 1763.785 | 0 | −0.2 | 4983 | 462.7 | 462.7 | 462.7 | 7.18 |
| 966 | R.HDWNYEHLFMDIK.N | | 874.398 | 2 | 1747.789 | 1747.790 | 0 | 0.3 | 4983 | 528.9 | 528.9 | 528.9 | 6.98 |
| 966 | R.HDWNYEHLFMDIK.N | | 874.398 | 2 | 1747.788 | 1747.790 | 0 | −1.0 | 4983 | 439.3 | 439.3 | 439.3 | 6.42 |
| 2192 | R.HDWNYEHLFMDIKNK.M | | 498.237 | 4 | 1989.927 | 1989.928 | 0 | −0.1 | 4983 | 354.0 | 354.0 | 354.0 | 5.82 |
| 966 | R.HDWNYEHLFMDIK.N | | 583.602 | 3 | 1748.793 | 1747.790 | 1 | 0.2 | 4983 | 339.8 | 339.8 | 339.8 | 5.71 |
| 1565 | R.HDWNYEHLFM[+15.995]DIK.N | M[+16] | 588.600 | 3 | 1763.784 | 1763.785 | 0 | −0.2 | 4983 | 350.5 | 350.5 | 350.5 | 5.32 |
| 966 | R.HDWNYEHLFMDIK.N | | 437.703 | 4 | 1747.789 | 1747.790 | 0 | −0.4 | 4983 | 341.7 | 341.7 | 341.7 | 5.32 |
| 1020 | K.HTIALWQFLSAHKSEQLLR.L | | 570.317 | 4 | 2278.247 | 2278.246 | 0 | 0.7 | 5074 | 706.0 | 706.0 | 706.0 | 12.70 |
| 2095 | K.HTIALWQFLSAHK.S | | 517.952 | 3 | 1551.841 | 1551.843 | 0 | −1.2 | 5074 | 412.4 | 412.4 | 412.4 | 6.76 |
| 2095 | K.HTIALWQFLSAHK.S | | 517.952 | 3 | 1551.842 | 1551.843 | 0 | −0.5 | 5074 | 399.0 | 399.0 | 399.0 | 6.64 |
| 903 | R.LHKEPFGEISSR.Y | | 467.249 | 3 | 1399.733 | 1399.733 | 0 | 0.1 | 5093 | 585.6 | 585.6 | 585.6 | 9.44 |
| 903 | R.LHKEPFGEISSR.Y | | 467.249 | 3 | 1399.734 | 1399.733 | 0 | 0.6 | 5093 | 576.2 | 576.2 | 576.2 | 9.44 |
| 903 | R.LHKEPFGEISSR.Y | | 467.249 | 3 | 1399.733 | 1399.733 | 0 | 0.1 | 5093 | 569.8 | 569.8 | 569.8 | 9.44 |
| 903 | R.LHKEPFGEISSR.Y | | 467.249 | 3 | 1399.733 | 1399.733 | 0 | −0.1 | 5093 | 546.1 | 546.1 | 546.1 | 8.71 |
| 903 | R.LHKEPFGEISSR.Y | | 467.249 | 3 | 1399.733 | 1399.733 | 0 | 0.1 | 5093 | 540.7 | 540.7 | 540.7 | 8.12 |
| 903 | R.LHKEPFGEISSR.Y | | 467.249 | 3 | 1399.733 | 1399.733 | 0 | 0.0 | 5093 | 519.4 | 519.4 | 519.4 | 7.94 |
| 903 | R.LHKEPFGEISSR.Y | | 467.249 | 3 | 1399.733 | 1399.733 | 0 | 0.0 | 5093 | 441.0 | 441.0 | 441.0 | 7.49 |
| 903 | R.LHKEPFGEISSR.Y | | 467.249 | 3 | 1399.732 | 1399.733 | 0 | −0.6 | 5093 | 483.3 | 483.3 | 483.3 | 7.35 |
| 903 | R.LHKEPFGEISSR.Y | | 467.249 | 3 | 1399.733 | 1399.733 | 0 | 0.2 | 5093 | 365.2 | 365.2 | 365.2 | 6.96 |
| 903 | R.LHKEPFGEISSR.Y | | 467.249 | 3 | 1399.732 | 1399.733 | 0 | −0.4 | 5093 | 399.8 | 399.8 | 399.8 | 6.56 |
| 903 | R.LHKEPFGEISSR.Y | | 700.370 | 2 | 1399.733 | 1399.733 | 0 | 0.3 | 5093 | 490.1 | 490.1 | 490.1 | 6.52 |
| 903 | R.LHKEPFGEISSR.Y | | 467.249 | 3 | 1399.732 | 1399.733 | 0 | −0.4 | 5093 | 424.5 | 424.5 | 424.5 | 6.49 |
| 903 | R.LHKEPFGEISSR.Y | | 700.370 | 2 | 1399.732 | 1399.733 | C | −0.4 | 5093 | 341.9 | 341.9 | 341.9 | 3.82 |
| 903 | R.LHKEPFGEISSR.Y | | 467.581 | 3 | 1400.728 | 1399.733 | 1 | −5.6 | 5093 | 357.4 | 357.4 | 357.4 | 2.63 |
| 903 | R.LHKEPFGEISSR.Y | | 467.581 | 3 | 1400.728 | 1399.733 | 1 | 5.6 | 5093 | 319.5 | 319.5 | 319.5 | 2.16 |
| 1820 | K.EPFGEISSR.Y | | 511.251 | 2 | 1021.495 | 1021.495 | 0 | −0.4 | 5096 | 372.6 | 372.6 | 372.6 | 6.64 |
| 1820 | K.EPFGEISSR.Y | | 511.251 | 2 | 1021.495 | 1021.495 | 0 | −0.1 | 5096 | 314.2 | 314.2 | 314.2 | 5.43 |
| 2609 | R.YKADLSPENAK.L | | 618.318 | 2 | 1235.628 | 1235.627 | 0 | 0.9 | 5105 | 535.1 | 535.1 | 535.1 | 8.71 |
| 1450 | K.LLSTFLNQTGLDAFLLELHEMIILK.L | | 958.203 | 3 | 2872.593 | 2872.589 | 0 | 1.4 | 5116 | 824.3 | 824.3 | 824.3 | 14.95 |
| 2126 | R.FRPQWSLR.D | | 545.301 | 2 | 1089.595 | 1089.595 | 0 | 0.1 | 5152 | 413.0 | 413.0 | 413.0 | 7.35 |
| 2126 | R.FRPQWSLR.D | | 545.301 | 2 | 1089.595 | 1089.595 | 0 | 0.4 | 5152 | 452.5 | 452.5 | 452.5 | 6.80 |
| 1786 | R.FRPQWSLRDTLVSYMQTK.E | | 564.795 | 4 | 2256.160 | 2256.159 | 0 | 0.1 | 5152 | 302.4 | 302.4 | 302.4 | 5.61 |
| 1786 | R.FRPQWSLRDTLVSYMQTK.E | | 752.725 | 3 | 2256.159 | 2256.159 | 0 | −0.3 | 5152 | 338.5 | 338.5 | 338.5 | 5.31 |
| 1540 | R.DTLVSYM[+15.995]QTK.E | M[+16] | 601.292 | 2 | 1201.577 | 1201.577 | 0 | 0.0 | 5160 | 549.8 | 546.8 | 546.8 | 8.39 |
| 861 | R.DTLVSYMQTK.E | | 593.295 | 2 | 1185.582 | 1185.582 | 0 | 0.2 | 5160 | 489.6 | 489.6 | 489.6 | 8.21 |
| 861 | R.DTLVSYMQTK.E | | 593.295 | 2 | 1185.582 | 1185.582 | 0 | 0.2 | 5160 | 475.0 | 475.0 | 475.0 | 8.05 |
| 861 | R.DTLVSYMQTK.E | | 593.295 | 2 | 1185.582 | 1185.582 | 0 | 0.4 | 5160 | 381.8 | 381.8 | 381.8 | 7.42 |
| 1734 | K.ESEILPEMASQFPEEILLASC[+57.021]VSVWK.T | C[+57] | 1496.740 | 2 | 2992.472 | 2992.468 | 0 | 1.2 | 5170 | 564.5 | 452.7 | 452.7 | 9.98 |
| 1734 | K.ESEILPEMASQFPEEILLASC[+57.021]VSVWK.T | C[+57] | 998.162 | 3 | 2992.472 | 2992.468 | 0 | 1.2 | 5170 | 582.1 | 574.8 | 574.8 | 9.46 |
| 1734 | K.ESEILPEMASQFPEEILLASC[+57.021]VSVWK.T | C[+57] | 998.162 | 3 | 2992.470 | 2992.468 | 0 | 0.7 | 5170 | 575.2 | 551.3 | 551.3 | 9.46 |
| 1144 | K.DNTVPLKLIALLANGEFHSGEQLGETLGMSR.A | | 828.433 | 4 | 3310.710 | 3310.710 | 0 | 0.0 | 5226 | 815.5 | 815.5 | 815.5 | 13.99 |
| 1144 | K.DNTVPLKLIALLANGEFHSGEQLGETLGMSR.A | | 828.682 | 4 | 3311.708 | 3310.710 | 1 | −1.8 | 5226 | 802.5 | 802.5 | 320.4 | 13.41 |
| 1144 | K.DNTVPLKLIALLANGEFHSGEQLGETLGMSR.A | | 1104.241 | 3 | 3310.709 | 3310.710 | 0 | −0.4 | 5226 | 774.1 | 774.1 | 774.1 | 12.41 |
| 1144 | K.DNTVPLKLIALLANGEFHSGEQLGETLGMSR.A | | 1104.242 | 3 | 3310.710 | 3310.710 | 0 | 0.0 | 5226 | 719.1 | 719.1 | 719.1 | 11.99 |
| 1575 | K.DNTVPLKLIALLANGEFHSGEQLGETLGM[+15.995]SR.A | M[+16] | 832.431 | 4 | 3326.702 | 3326.705 | ) | 0.8 | 5226 | 675.8 | 580.0 | 580.0 | 10.41 |
| 1144 | K.DNTVPLKLIALLANGEFHSGEQLGETLGMSR.A | | 1104.241 | 3 | 3310.708 | 3310.710 | 0 | 0.6 | 5226 | 612.9 | 612.9 | 612.9 | 9.41 |
| 1144 | K.DNTVPLKLIALLANGEFHSGEQLGETLGMSR.A | | 662.948 | 5 | 3310.710 | 3310.710 | 0 | 0.0 | 5226 | 589.5 | 589.5 | 589.5 | 8.47 |
| 1575 | K.DNTVPLKLIALLANGEFHSGEQLGETLGM[+15.995]SR.A | M[+16] | 832.431 | 4 | 3326.703 | 3326.705 | 0 | −0.5 | 5226 | 569.1 | 569.1 | 569.1 | 7.52 |
| 1144 | K.DNTVPLKLIALLANGEFHSGEQLGETLGMSR.A | | 828.433 | 4 | 3310.710 | 3310.710 | 0 | 0.1 | 5226 | 497.6 | 497.6 | 497.6 | 7.48 |
| 1144 | K.DNTVPLKLIALLANGEFHSGEQLGETLGMSR.A | | 828.433 | 4 | 3310.710 | 3310.710 | 0 | 0.1 | 5226 | 472.2 | 458.4 | 458.4 | 6.85 |
| 1575 | K.DNTVPLKLIALLANGEFHSGEQLGETLGM[+15.995]SR.A | M[+16] | 832.432 | 4 | 3326.706 | 3326.705 | 0 | 0.2 | 5226 | 349.2 | 349.2 | 349.2 | 5.37 |
| 1515 | K.LIALLAN[+0.984]GEFHSGEQLGETLGMSR.A | N[+1] | 848.764 | 3 | 2544.277 | 2544.276 | 0 | 0.4 | 5233 | 647.4 | 569.2 | 81.4 | 10.98 |
| 1515 | K.LIALLAN[+0.984]GEFHSGEQLGETLGMSR.A | N[+1] | 848.764 | 3 | 2544.279 | 2544.276 | 0 | 0.9 | 5233 | 562.6 | 524.8 | 260.9 | 9.98 |
| 874 | K.LIALLANGEFHSGEQLGETLGMSR.A | | 848.436 | 3 | 2543.292 | 2543.292 | 0 | −0.2 | 5233 | 388.5 | 388.5 | 388.5 | 7.97 |
| 874 | K.LIALLANGEFHSGEQLGETLGMSR.A | | 848.436 | 3 | 2543.295 | 2543.292 | 0 | 0.9 | 5233 | 377.9 | 377.9 | 377.9 | 7.49 |
| 874 | K.LIALLANGEFHSGEQLGETLGMSR.A | | 848.435 | 3 | 2543.291 | 2543.292 | 0 | 0.5 | 5233 | 408.5 | 388.0 | 388.0 | 7.21 |
| 874 | K.LIALLANGEFHSGEQLGETLGMSR.A | | 848.436 | 3 | 2543.293 | 2543.292 | 0 | 0.1 | 5233 | 321.6 | 312.1 | 312.1 | 5.96 |
| 852 | R.AAINKHIQTLR.D | | 422.255 | 3 | 1264.750 | 1264.748 | 0 | 1.2 | 5257 | 544.1 | 544.1 | 544.1 | 8.71 |
| 852 | R.AAINKHIQTLR.D | | 422.255 | 3 | 1264.749 | 1264.748 | 0 | 0.7 | 5257 | 527.0 | 487.0 | 487.0 | 8.71 |
| 852 | R.AAINKHIQTLR.D | | 422.255 | 3 | 1264.750 | 1264.748 | 0 | 1.1 | 5257 | 515.8 | 472.3 | 472.3 | 8.53 |
| 852 | R.AAINKHIQTLR.D | | 422.255 | 3 | 1264.749 | 1264.748 | 0 | 0.6 | 5257 | 497.4 | 497.4 | 497.4 | 8.53 |
| 852 | R.AAINKHIQTLR.D | | 422.254 | 3 | 1264.749 | 1264.748 | 0 | 0.2 | 5257 | 483.5 | 471.1 | 471.1 | 8.53 |
| 852 | R.AAINKHIQTLR.D | | 422.255 | 3 | 1264.750 | 1264.748 | 0 | 1.3 | 5257 | 411.9 | 411.9 | 411.9 | 8.15 |
| 1796 | R.AAINKHIQTLRDWGVDVFTVPGK.G | | 642.104 | 4 | 2565.394 | 2565.394 | 0 | 0.1 | 5257 | 329.0 | 329.0 | 329.0 | 5.73 |
| 1796 | R.AAINKHIQTLRDWGVDVFTVPGK.G | | 855.803 | 3 | 2565.393 | 2565.394 | 0 | 0.1 | 5257 | 303.1 | 303.1 | 303.1 | 3.84 |
| 826 | K.HIQTLRDWGVDVFTVPGKGYSLPEPIPLLNAK.Q | | 890.988 | 4 | 3560.928 | 3560.926 | 0 | 0.5 | 5262 | 638.5 | 638.5 | 638.5 | 9.64 |
| 826 | K.HIQTLRDWGVDVFTVPGKGYSLPEPIPLLNAK.Q | | 890.987 | 4 | 3560.927 | 3560.926 | 0 | 0.1 | 5262 | 608.7 | 608.7 | 608.7 | 9.64 |
| 826 | K.HIQTLRDWGVDVFTVPGKGYSLPEPIPLLNAK.Q | | 890.987 | 4 | 3560.927 | 3560.926 | 0 | 0.1 | 5262 | 564.3 | 564.3 | 564.3 | 8.64 |
| 826 | K.HIQTLRDWGVDVFTVPGKGYSLPEPIPLLNAK.Q | | 712.992 | 5 | 3560.929 | 3560.926 | 0 | 0.7 | 5262 | 544.0 | 544.0 | 544.0 | 7.91 |
| 1810 | K.HIQTLRDWGVDVFTVPGK.G | | 690.038 | 3 | 2068.099 | 2068.097 | 0 | 0.5 | 5262 | 394.3 | 394.3 | 394.3 | 7.40 |
| 826 | K.HIQTLRDWGVDVFTVPGKGYSLPEPIPLLNAK.Q | | 712.991 | 5 | 3560.927 | 3560.926 | 0 | 0.2 | 5262 | 403.1 | 403.1 | 403.1 | 7.35 |
| 826 | K.HIQTLRDWGVDVFTVPGKGYSLPEPIPLLNAK.Q | | 712.991 | 5 | 3560.928 | 3560.926 | 0 | 0.4 | 5262 | 422.6 | 422.6 | 422.6 | 6.75 |
| 826 | K.HIQTLRDWGVDVFTVPGKGYSLPEPIPLLNAK.Q | | 594.327 | 6 | 3560.924 | 3560.926 | 0 | 0.7 | 5262 | 376.4 | 376.4 | 376.4 | 6.13 |
| 826 | K.HIQTLRDWGVDVFTVPGKGYSLPEPIPLLNAK.Q | | 594.328 | 6 | 3560.929 | 3560.926 | 0 | 0.6 | 5262 | 389.3 | 389.3 | 389.3 | 6.12 |
| 1810 | K.HIQTLRDWGVDVFTVPGK.G | | 690.038 | 3 | 2068.098 | 2068.097 | 0 | 0.3 | 5262 | 332.7 | 332.7 | 332.7 | 5.89 |
| 826 | K.HIQTLRDWGVDVFTVPGKGYSLPEPIPLLNAK.Q | | 712.991 | 5 | 3560.927 | 3560.926 | 0 | 0.1 | 5262 | 343.1 | 343.1 | 343.1 | 5.31 |
| 826 | K.HIQTLRDWGVDVFTVPGKGYSLPEPIPLLNAK.Q | | 712.992 | 5 | 3560.929 | 3560.926 | 0 | 0.8 | 5262 | 308.2 | 308.2 | 308.2 | 4.55 |
| 1109 | R.DWGVDVFTVPGKGYSLPEPIPLLNAK.Q | | 938.170 | 3 | 2812.496 | 2812.492 | 0 | 1.5 | 5268 | 838.9 | 838.9 | 838.9 | 14.70 |
| 1510 | R.DWGVDVFTVPGKGYSLPEPIPLLN[+0.984]AK.Q | N[+1] | 938.498 | 3 | 2813.479 | 2813.476 | 0 | 1.0 | 5268 | 807.0 | 807.0 | 502.2 | 14.70 |
| 1109 | R.DWGVDVFTVPGKGYSLPEPIPLLNAK.Q | | 1406.751 | 2 | 2812.495 | 2812.492 | 0 | 0.9 | 5268 | 722.6 | 722.6 | 722.6 | 12.70 |
| 1109 | R.DWGVDVFTVPGKGYSLPEPIPLLNAK.Q | | 703.879 | 4 | 2812.495 | 2812.492 | 0 | 1.2 | 5268 | 560.6 | 560.6 | 560.6 | 9.19 |
| 1510 | R.DWGVDVFTVPGKGYSLPEPIPLLN[+0.984]AK.Q | N[+1] | 938.497 | 3 | 2813.478 | 2813.476 | 0 | 0.6 | 5268 | 577.7 | 577.7 | 215.8 | 8.82 |
| 931 | R.DWGVDVFTVPGK.G | | 660.335 | 2 | 1319.663 | 1319.663 | 0 | 0.3 | 5268 | 535.9 | 535.9 | 535.9 | 8.39 |
| 1510 | R.DWGVDVFTVPGKGYSLPEPIPLLN[+0.984]AK.Q | N[+1] | 938.498 | 3 | 2813.479 | 2813.476 | 0 | 1.0 | 5268 | 488.5 | 488.5 | 182.6 | 7.72 |
| 931 | R.DWGVDVFTVPGK.G | | 660.336 | 2 | 1319.664 | 1319.663 | 0 | 0.5 | 5268 | 425.7 | 425.7 | 425.7 | 7.53 |
| 1109 | R.DWGVDVFTVPGKGYSLPEPIPLLNAK.Q | | 938.163 | 3 | 2812.474 | 2812.492 | 0 | −6.4 | 5268 | 587.1 | 566.6 | 566.6 | 7.49 |
| 931 | R.DWGVDVFTVPGK.G | | 660.335 | 2 | 1319.663 | 1319.663 | 0 | 0.1 | 5268 | 374.9 | 374.9 | 374.9 | 7.42 |
| 931 | R.DWGVDVFTVPGK.G | | 440.559 | 3 | 1319.663 | 1319.663 | 0 | 0.2 | 5268 | 403.1 | 403.1 | 403.1 | 6.83 |
| 1109 | R.DWGVDVFTVPGKGYSLPEPIPLLNAK.Q | | 703.879 | 4 | 2812.493 | 2812.492 | 0 | 0.4 | 5268 | 324.4 | 324.4 | 324.4 | 5.38 |
| 2046 | R.DWGVDVFTVPGKGYSLPEPIPLLNAKQILGQLDGGSVAVLPVVDS | | 1133.798 | 5 | 5664.963 | 5664.000 | 1 | −7.2 | 5268 | 374.2 | 374.2 | 30.5 | 3.02 |
| | TNQYLLDR.I | | | | | | | | | | | | |
| 950 | K.GYSLPEPIPLLNAK.Q | | 756.427 | 2 | 1511.846 | 1511.847 | 0 | 0.5 | 5280 | 669.9 | 669.9 | 669.9 | 11.13 |
| 950 | K.GYSLPEPIPLLNAK.Q | | 756.427 | 2 | 1511.847 | 1511.847 | 0 | 0.1 | 5280 | 569.8 | 569.8 | 569.8 | 9.72 |
| 950 | K.GYSLPEPIPLLNAK.Q | | 756.427 | 2 | 1511.847 | 1511.847 | 0 | 0.1 | 5280 | 507.5 | 507.5 | 507.5 | 8.80 |
| 950 | K.GYSLPEPIPLLNAK.Q | | 504.620 | 3 | 1511.847 | 1511.847 | 0 | −0.1 | 5280 | 535.2 | 535.2 | 535.2 | 8.47 |
| 950 | K.GYSLPEPIPLLNAK.Q | | 756.427 | 2 | 1511.847 | 1511.847 | 0 | −0.1 | 5280 | 415.2 | 415.2 | 415.2 | 8.42 |
| 950 | K.GYSLPEPIPLLNAK.Q | | 756.427 | 2 | 1511.846 | 1511.847 | 0 | −0.5 | 5280 | 477.5 | 477.5 | 477.5 | 8.06 |
| 950 | K.GYSLPEPIPLLNAK.Q | | 756.427 | 2 | 1511.847 | 1511.847 | 0 | 0.3 | 5280 | 392.8 | 392.8 | 392.8 | 7.71 |
| 964 | K.QILGQLDGGSVAVLPVVDSTNQYLLDRIGELK.S | | 682.977 | 5 | 3410.855 | 3410.853 | 0 | 0.5 | 5294 | 882.2 | 882.2 | 882.2 | 14.48 |
| 964 | K.QILGQLDGGSVAVLPVVDSTNQYLLDRIGELK.S | | 853.469 | 4 | 3410.854 | 3410.853 | 0 | 0.4 | 5294 | 871.4 | 871.4 | 871.4 | 14.48 |
| 964 | K.QILGQLDGGSVAVLPVVDSTNQYLLDRIGELK.S | | 1137.622 | 3 | 3410.852 | 3410.853 | 0 | −0.2 | 5294 | 885.6 | 885.6 | 885.6 | 14.41 |
| 964 | K.QILGQLDGGSVAVLPVVDSTNQYLLDRIGELK.S | | 1137.622 | 3 | 3410.852 | 3410.853 | 0 | 0.4 | 5294 | 869.1 | 869.1 | 869.1 | 14.41 |
| 964 | K.QILGQLDGGSVAVLPVVDSTNQYLLDRIGELK.S | | 1137.622 | 3 | 3410.851 | 3410.853 | 0 | −0.7 | 5294 | 861.5 | 861.5 | 861.5 | 14.41 |
| 964 | K.QILGQLDGGSVAVLPVVDSTNQYLLDRIGELK.S | | 1137.624 | 3 | 3410.857 | 3410.853 | 0 | 1.3 | 5294 | 822.3 | 822.3 | 822.3 | 13.99 |
| 893 | K.QILGQLDGGSVAVLPVVDSTNQYLLDR.I | | 1435.767 | 2 | 2870.526 | 2870.526 | 0 | 0.2 | 5294 | 769.2 | 769.2 | 769.2 | 13.98 |
| 964 | K.QILGQLDGGSVAVLPVVDSTNQYLLDRIGELK.S | | 1137.624 | 3 | 3410.856 | 3410.853 | 0 | 0.9 | 5294 | 769.7 | 769.7 | 769.7 | 12.99 |
| 893 | K.QILGQLDGGSVAVLPVVDSTNQYLLDR.I | | 1435.768 | 2 | 2870.528 | 2870.526 | 0 | 0.7 | 5294 | 714.6 | 714.6 | 714.6 | 12.98 |
| 893 | K.QILGQLDGGSVAVLPVVDSTNQYLLDR.I | | 1435.766 | 2 | 2870.525 | 2870.526 | 0 | −0.2 | 5294 | 701.8 | 701.8 | 701.8 | 12.98 |
| 893 | K.QILGQLDGGSVAVLPVVDSTNQYLLDR.I | | 1435.767 | 2 | 2870.527 | 2870.526 | 0 | 0.5 | 5294 | 697.3 | 697.3 | 697.3 | 11.98 |
| 964 | K.QILGQLDGGSVAVLPVVDSTNQYLLDRIGELK.S | | 853.469 | 4 | 3410.853 | 3410.853 | 0 | 0.1 | 5294 | 737.0 | 737.0 | 737.0 | 11.47 |
| 893 | K.QILGQLDGGSVAVLPVVDSTNQYLLDR.I | | 1435.768 | 2 | 2870.529 | 2870.526 | 0 | 0.9 | 5294 | 634.7 | 634.7 | 634.7 | 10.98 |
| 893 | K.QILGQLDGGSVAVLPVVDSTNQYLLDR.I | | 1435.769 | 2 | 2870.531 | 2870.526 | 0 | 1.7 | 5294 | 618.8 | 618.8 | 618.8 | 10.98 |
| 893 | K.QILGQLDGGSVAVLPVVDSTNQYLLDR.I | | 1435.766 | 2 | 2870.525 | 2870.526 | 0 | −0.2 | 5294 | 603.2 | 603.2 | 603.2 | 10.98 |
| 964 | K.QILGQLDGGSVAVLPVVDSTNQYLLDRIGELK.S | | 853.469 | 4 | 3410.854 | 3410.853 | 0 | 0.3 | 5294 | 673.1 | 673.1 | 673.1 | 10.47 |
| 893 | K.QILGQLDGGSVAVLPVVDSTNQYLLDR.I | | 957.515 | 3 | 2870.529 | 2870.526 | 0 | 1.1 | 5294 | 633.2 | 633.2 | 633.2 | 10.46 |
| 893 | K.QILGQLDGGSVAVLPVVDSTNQYLLDR.I | | 957.514 | 3 | 2870.526 | 2870.526 | 0 | 0.1 | 5294 | 631.7 | 631.7 | 631.7 | 10.46 |
| 893 | K.QILGQLDGGSVAVLPVVDSTNQYLLDR.I | | 957.513 | 3 | 2870.526 | 2870.526 | 0 | −0.1 | 5294 | 618.3 | 618.3 | 618.3 | 10.46 |
| 893 | K.QILGQLDGGSVAVLPVVDSTNQYLLDR.I | | 1435.767 | 2 | 2870.527 | 2870.526 | 0 | 0.2 | 5294 | 597.9 | 597.9 | 597.9 | 9.98 |
| 893 | K.QILGQLDGGSVAVLPVVDSTNQYLLDR.I | | 1435.767 | 2 | 2870.526 | 2870.526 | 0 | 0.0 | 5294 | 594.9 | 594.9 | 594.9 | 9.98 |
| 893 | K.QILGQLDGGSVAVLPVVDSTNQYLLDR.I | | 718.387 | 4 | 2870.527 | 2870.526 | 0 | 0.3 | 5294 | 595.3 | 595.3 | 595.3 | 9.46 |
| 1505 | K.QILGQLDGGSVAVLPVVDSTN[+0.984]QYLLDR.I | N[+1] | 957.842 | 3 | 2871.512 | 2871.510 | 0 | 0.9 | 5294 | 588.8 | 588.8 | 155.0 | 9.46 |
| 893 | K.QILGQLDGGSVAVLPVVDSTNQYLLDR.I | | 718.387 | 4 | 2870.526 | 2870.526 | 0 | 0.1 | 5294 | 576.0 | 576.0 | 576.0 | 9.46 |
| 893 | K.QILGQLDGGSVAVLPVVDSTNQYLLDR.I | | 957.515 | 3 | 2870.530 | 2870.526 | 0 | 1.3 | 5294 | 571.7 | 571.7 | 571.7 | 9.46 |
| 893 | K.QILGQLDGGSVAVLPVVDSTNQYLLDR.I | | 957.513 | 3 | 2870.525 | 2870.526 | 0 | −0.1 | 5294 | 569.4 | 569.4 | 569.4 | 9.46 |
| 893 | K.QILGQLDGGSVAVLPVVDSTNQYLLDR.I | | 957.513 | 3 | 2870.525 | 2870.526 | 0 | 0.2 | 5294 | 597.3 | 597.3 | 597.3 | 8.88 |
| 893 | K.QILGQLDGGSVAVLPVVDSTNQYLLDR.I | | 957.514 | 3 | 2870.526 | 2870.526 | 0 | 0.0 | 5294 | 558.0 | 558.0 | 558.0 | 8.73 |
| 893 | K.QILGQLDGGSVAVLPVVDSTNQYLLDR.I | | 718.387 | 4 | 2870.526 | 2870.526 | 0 | −0.1 | 5294 | 549.0 | 549.0 | 549.0 | 8.73 |
| 893 | K.QILGQLDGGSVAVLPVVDSTNQYLLDR.I | | 957.514 | 3 | 2870.528 | 2870.526 | 0 | 0.8 | 5294 | 529.8 | 529.8 | 529.8 | 8.73 |
| 893 | K.QILGQLDGGSVAVLPVVDSTNQYLLDR.I | | 957.514 | 3 | 2870.529 | 2870.526 | 0 | 1.0 | 5294 | 522.2 | 522.2 | 522.2 | 8.73 |
| 1505 | K.QILGQLDGGSVAVLPVVDSTN[+0.984]QYLLDR.I | N[+1] | 957.842 | 3 | 2871.511 | 2871.510 | 0 | 0.5 | 5294 | 514.7 | 514.7 | 185.6 | 8.55 |
| 893 | K.QILGQLDGGSVAVLPVVDSTNQYLLDR.I | | 957.514 | 3 | 2870.526 | 2870.526 | 0 | 0.1 | 5294 | 510.5 | 510.5 | 510.5 | 8.55 |
| 964 | K.QILGQLDGGSVAVLPVVDSTNQYLLDRIGELK.S | | 1137.625 | 3 | 3410.859 | 3410.853 | 0 | 1.8 | 5294 | 557.5 | 557.5 | 557.5 | 8.26 |
| 893 | K.QILGQLDGGSVAVLPVVDSTNQYLLDR.I | | 957.514 | 3 | 2870.527 | 2870.526 | 0 | 0.4 | 5294 | 521.3 | 521.3 | 521.3 | 8.13 |
| 893 | K.QILGQLDGGSVAVLPVVDSTNQYLLDR.I | | 957.514 | 3 | 2870.527 | 2870.526 | 0 | 0.3 | 5294 | 485.4 | 485.4 | 485.4 | 7.95 |
| 964 | K.QILGQLDGGSVAVLPVVDSTNQYLLDRIGELK.S | | 853.470 | 4 | 3410.857 | 3410.853 | 0 | 1.2 | 5294 | 550.8 | 550.8 | 550.8 | 7.74 |
| 893 | K.QILGQLDGGSVAVLPVVDSTNQYLLDR.I | | 957.513 | 3 | 2870.525 | 2870.526 | 0 | 0.4 | 5294 | 473.2 | 473.2 | 473.2 | 6.92 |
| 1505 | K.QILGQLDGGSVAVLPVVDSTN[+0.984]QYLLDR.I | N[+1] | 958.516 | 3 | 2873.533 | 2871.510 | 2 | 5.8 | 5294 | 474.8 | 474.8 | 474.8 | 6.24 |
| 964 | K.QILGQLDGGSVAVLPVVDSTNQYLLDRIGELK.S | | 853.470 | 4 | 3410.856 | 3410.853 | 0 | 0.9 | 5294 | 428.0 | 428.0 | 428.0 | 6.11 |
| 893 | K.QILGQLDGGSVAVLPVVDSTNQYLLDR.I | | 957.508 | 3 | 2870.508 | 2870.526 | 0 | 6.1 | 5294 | 415.5 | 415.5 | 415.5 | 5.95 |
| 964 | K.QILGQLDGGSVAVLPVVDSTNQYLLDRIGELK.S | | 1137.615 | 3 | 3410.829 | 3410.853 | 0 | −6.9 | 5294 | 551.3 | 551.3 | 551.3 | 5.86 |
| 893 | K.QILGQLDGGSVAVLPVVDSTNQYLLDR.I | | 718.387 | 4 | 2870.526 | 2870.526 | 0 | 0.2 | 5294 | 326.2 | 326.2 | 326.2 | 5.65 |
| 893 | K.QILGQLDGGSVAVLPVVDSTNQYLLDR.I | | 957.850 | 3 | 2871.535 | 2870.526 | 1 | 2.0 | 5294 | 314.1 | 314.1 | 243.0 | 5.56 |
| 964 | K.QILGQLDGGSVAVLPVVDSTNQYLLDRIGELK.S | | 853.461 | 4 | 3410.822 | 3410.853 | 0 | −9.2 | 5294 | 475.2 | 475.2 | 475.2 | 4.77 |
| 1698 | K.SGDAC[+57.021]IAEYQQAGR.G | C[+57] | 763.338 | 2 | 1525.670 | 1525.670 | 0 | 0.3 | 5326 | 741.7 | 741.7 | 741.7 | 12.13 |
| 1711 | K.SGDAC[+57.021]IAEYQQAGRGSR.G | C[+57] | 609.280 | 3 | 1825.825 | 1825.825 | 0 | 0.2 | 5326 | 496.9 | 496.9 | 496.9 | 7.91 |
| 822 | K.RGPAAIGLGPVIGIVMAEALR.K | | 687.738 | 3 | 2061.200 | 2061.200 | 0 | 0.1 | 5364 | 537.1 | 537.1 | 537.1 | 9.25 |
| 822 | K.RGPAAIGLGPVIGIVMAEALR.K | | 687.738 | 3 | 2061.200 | 2061.200 | 0 | 0.1 | 5364 | 432.2 | 432.2 | 432.2 | 7.80 |
| 2620 | R.GPAAIGLGPVIGIVMAEALR.K | | 953.053 | 2 | 1905.099 | 1905.099 | 0 | 0.2 | 5365 | 503.1 | 503.1 | 503.1 | 9.07 |
| 2620 | R.GPAAIGLGPVIGIVMAEALR.K | | 635.705 | 3 | 1905.100 | 1905.099 | 0 | 0.2 | 5365 | 551.8 | 551.8 | 551.8 | 8.73 |
| 919 | R.VKWPNDLYLQDRK.L | | 419.480 | 4 | 1674.896 | 1674.896 | 0 | 0.0 | 5393 | 547.3 | 547.3 | 547.3 | 8.36 |
| 912 | R.VKWPNDLYLQDR.K | | 516.272 | 3 | 1546.801 | 1546.801 | 0 | 0.0 | 5393 | 527.1 | 527.1 | 527.1 | 8.12 |
| 912 | R.VKWPNDLYLQDR.K | | 516.272 | 3 | 1546.801 | 1546.801 | 0 | 0.1 | 5393 | 526.6 | 511.9 | 511.9 | 8.12 |
| 912 | R.VKWPNDLYLQDR.K | | 773.904 | 2 | 1546.801 | 1546.801 | 0 | 0.4 | 5393 | 485.1 | 485.1 | 485.1 | 7.95 |
| 912 | R.VKWPNDLYLQDR.K | | 516.272 | 3 | 1546.801 | 1546.801 | 0 | 0.1 | 5393 | 489.2 | 428.5 | 428.5 | 7.94 |
| 912 | R.VKWPNDLYLQDR.K | | 516.272 | 3 | 1546.802 | 1546.801 | 0 | 0.2 | 5393 | 471.6 | 471.6 | 471.6 | 7.78 |
| 919 | R.VKWPNDLYLQDRK.L | | 558.970 | 3 | 1674.896 | 1674.896 | 0 | −0.3 | 5393 | 552.9 | 552.9 | 552.9 | 7.18 |
| 912 | R.VKWPNDLYLQDR.K | | 773.904 | 2 | 1546.801 | 1546.801 | 0 | 0.3 | 5393 | 450.7 | 363.9 | 363.9 | 6.90 |
| 919 | R.VKWPNDLYLQDRK.L | | 558.970 | 3 | 1674.896 | 1674.896 | 0 | −0.3 | 5393 | 495.7 | 495.7 | 495.7 | 6.71 |
| 912 | R.VKWPNDLYLQDR.K | | 516.606 | 3 | 1547.804 | 1546.801 | 1 | −0.2 | 5393 | 532.6 | 532.6 | 532.6 | 6.45 |
| 857 | R.RVEESVVNQGWITLQEAGINLDRNTLAATLIR.E | | 895.737 | 4 | 3579.927 | 3579.924 | 0 | 0.7 | 5435 | 802.6 | 700.8 | 700.8 | 13.99 |
| 857 | R.RVEESVVNQGWITLQEAGINLDRNTLAATLIR.E | | 895.736 | 4 | 3579.923 | 3579.924 | 0 | 0.3 | 5435 | 836.9 | 795.6 | 795.6 | 13.41 |
| 857 | R.RVEESVVNQGWITLQEAGINLDRNTLAATLIR.E | | 895.736 | 4 | 3579.923 | 3579.924 | 0 | −0.2 | 5435 | 764.5 | 756.6 | 756.6 | 12.41 |
| 857 | R.RVEESVVNQGWITLQEAGINLDRNTLAATLIR.E | | 716.792 | 5 | 3579.931 | 3579.924 | 0 | 2.0 | 5435 | 726.6 | 645.5 | 645.5 | 11.47 |
| 857 | R.RVEESVVNQGWITLQEAGINLDRNTLAATLIR.E | | 895.736 | 4 | 3579.923 | 3579.924 | 0 | 0.3 | 5435 | 681.9 | 660.6 | 660.6 | 10.41 |
| 1292 | R.VEESVVNQGWITLQEAGINLDRNTLAATLIR.E | | 1141.947 | 3 | 3423.827 | 3423.823 | 0 | 1.0 | 5436 | 868.3 | 868.3 | 868.3 | 15.00 |
| 1292 | R.VEESVVNQGWITLQEAGINLDRNTLAATLIR.E | | 856.711 | 4 | 3423.824 | 3423.823 | 0 | 0.3 | 5436 | 771.1 | 771.1 | 771.1 | 12.47 |
| 1292 | R.VEESVVNQGWITLQEAGINLDRNTLAATLIR.E | | 685.571 | 5 | 3423.825 | 3423.823 | 0 | 0.7 | 5436 | 604.0 | 604.0 | 604.0 | 9.47 |
| 2188 | R.NTLAATLIR.E | | 486.795 | 2 | 972.584 | 972.584 | 0 | 0.1 | 5458 | 420.4 | 420.4 | 420.4 | 7.53 |
| 2188 | R.NTLAATLIR.E | | 486.795 | 2 | 972.584 | 972.584 | 0 | 0.1 | 5458 | 423.7 | 423.7 | 423.7 | 7.35 |
| 1793 | R.ELRAALELFEQEGLAPYLPR.W | | 772.752 | 3 | 2316.242 | 2315.239 | 1 | −0.2 | 5467 | 532.6 | 532.6 | 532.6 | 8.97 |
| 1032 | R.AALELFEQEGLAPYLPRWEK.L | | 787.414 | 3 | 2360.228 | 2360.229 | 0 | −0.3 | 5470 | 752.6 | 752.6 | 752.6 | 13.12 |
| 1914 | R.AALELFEQEGLAPYLPR.W | | 959.010 | 2 | 1917.012 | 1917.012 | 0 | 0.0 | 5470 | 467.6 | 467.6 | 467.6 | 8.91 |
| 1914 | R.AALELFEQEGLAPYLPR.W | | 639.676 | 3 | 1917.013 | 1917.012 | 0 | 0.5 | 5470 | 449.3 | 449.3 | 449.3 | 7.50 |
| 1914 | R.AALELFEQEGLAPYLPR.W | | 639.676 | 3 | 1917.013 | 1917.012 | 0 | 0.5 | 5470 | 322.3 | 322.3 | 322.3 | 5.65 |
| 973 | R.WEKLDNFINRPVK.L | | 415.481 | 4 | 1658.901 | 1658.901 | 0 | 0.1 | 5487 | 464.4 | 464.4 | 464.4 | 7.78 |
| 973 | R.WEKLDNFINRPVK.L | | 553.638 | 3 | 1658.900 | 1658.901 | 0 | 0.9 | 5487 | 532.5 | 532.5 | 532.5 | 7.53 |
| 973 | R.WEKLDNFINRPVK.L | | 415.481 | 4 | 1658.902 | 1658.901 | 0 | 0.1 | 5487 | 330.7 | 330.7 | 330.7 | 5.63 |
| 973 | R.WEKLDNFINRPVK.L | | 415.481 | 4 | 1658.901 | 1658.901 | 0 | 0.1 | 5487 | 321.3 | 321.3 | 321.3 | 5.44 |
| 820 | K.LDNFINRPVKLIIGDKEIFGISR.G | | 532.309 | 5 | 2657.514 | 2657.514 | 0 | 0.2 | 5490 | 484.3 | 484.3 | 484.3 | 8.44 |
| 820 | K.LDNFINRPVKLIIGDKEIFGISR.G | | 532.309 | 5 | 2657.515 | 2657.514 | 0 | 0.5 | 5490 | 419.5 | 419.5 | 419.5 | 7.47 |
| 820 | K.LDNFINRPVKLIIGDKEIFGISR.G | | 665.134 | 4 | 2657.515 | 2657.514 | 0 | 0.5 | 5490 | 473.6 | 468.2 | 468.2 | 7.40 |
| 1931 | K.LDNFINRPVK.L | | 405.900 | 3 | 1215.684 | 1215.684 | 0 | 0.0 | 5490 | 404.0 | 404.0 | 404.0 | 7.35 |
| 820 | K.LDNFINRPVKLIIGDKEIFGISR.G | | 665.134 | 4 | 2657.515 | 2657.514 | 0 | 0.5 | 5490 | 423.8 | 423.8 | 423.8 | 7.17 |
| 820 | K.LDNFINRPVKLIIGDKEIFGISR.G | | 665.134 | 4 | 2657.515 | 2657.514 | 0 | 0.6 | 5490 | 417.2 | 417.2 | 417.2 | 7.17 |
| 1931 | K.LDNFINRPVK.L | | 608.346 | 2 | 1215.684 | 1215.684 | 0 | 0.2 | 5490 | 450.4 | 450.4 | 450.4 | 6.99 |
| 1931 | K.LDNFINRPVK.L | | 608.346 | 2 | 1215.684 | 1215.684 | 0 | 0.1 | 5490 | 318.4 | 195.9 | 195.9 | 5.08 |
| 983 | K.LIIGDKEIFGISR.G | | 730.927 | 2 | 1460.847 | 1460.847 | 0 | 0.1 | 5500 | 654.7 | 654.7 | 654.7 | 11.44 |
| 983 | K.LIIGDKEIFGISR.G | | 487.620 | 3 | 1460.847 | 1460.847 | 0 | −0.4 | 5500 | 655.8 | 655.8 | 655.8 | 10.86 |
| 983 | K.LIIGDKEIFGISR.G | | 487.621 | 3 | 1460.847 | 1460.847 | 0 | −0.2 | 5500 | 619.8 | 619.8 | 619.8 | 9.86 |
| 983 | K.LIIGDKEIFGISR.G | | 487.621 | 3 | 1460.847 | 1460.847 | 0 | 0.2 | 5500 | 597.7 | 597.7 | 597.7 | 9.44 |
| 983 | K.LIIGDKEIFGISR.G | | 487.621 | 3 | 1460.847 | 1460.847 | 0 | 0.0 | 5500 | 563.0 | 563.0 | 563.0 | 9.44 |
| 983 | K.LIIGDKEIFGISR.G | | 487.621 | 3 | 1460.848 | 1460.847 | 0 | 0.3 | 5500 | 581.7 | 581.7 | 581.7 | 8.85 |
| 983 | K.LIIGDKEIFGISR.G | | 487.620 | 3 | 1460.845 | 1460.847 | 0 | −1.2 | 5500 | 524.1 | 524.1 | 524.1 | 7.24 |
| 983 | K.LIIGDKEIFGISR.G | | 487.621 | 3 | 1460.847 | 1460.847 | 0 | −0.2 | 5500 | 389.8 | 389.8 | 389.8 | 7.14 |
| 983 | K.LIIGDKEIFGISR.G | | 487.620 | 3 | 1460.846 | 1460.847 | 0 | −0.5 | 5500 | 427.5 | 427.5 | 427.5 | 6.97 |
| 837 | R.GIDKQGALLLEQDGVIKPWMGGEISLR.S | | 975.196 | 3 | 2923.573 | 2923.571 | 0 | 0.7 | 5513 | 886.2 | 886.2 | 886.2 | 15.65 |
| 837 | R.GIDKQGALLLEQDGVIKPWMGGEISLR.S | | 975.196 | 3 | 2923.572 | 2923.571 | 0 | 0.4 | 5513 | 797.6 | 797.6 | 797.6 | 13.70 |
| 837 | R.GIDKQGALLLEQDGVIKPWMGGEISLR.S | | 975.195 | 3 | 2923.571 | 2923.571 | 0 | −0.1 | 5513 | 791.3 | 791.3 | 791.3 | 13.70 |
| 1612 | R.GIDKQGALLLEQDGVIKPWM[+15.995]GGEISLR.S | M[+16] | 980.527 | 3 | 2939.567 | 2939.566 | 0 | 0.3 | 5513 | 791.2 | 791.2 | 791.2 | 13.70 |
| 837 | R.GIDKQGALLLEQDGVIKPWMGGEISLR.S | | 975.529 | 3 | 2924.572 | 2923.571 | 1 | −0.9 | 5513 | 750.2 | 750.2 | 750.2 | 13.12 |
| 837 | R.GIDKQGALLLEQDGVIKPWMGGEISLR.S | | 731.648 | 4 | 2923.571 | 2923.571 | 0 | 0.0 | 5513 | 722.5 | 722.5 | 722.5 | 12.70 |
| 837 | R.GIDKQGALLLEQDGVIKPWMGGEISLR.S | | 731.648 | 4 | 2923.572 | 2923.571 | 0 | 0.2 | 5513 | 710.8 | 710.8 | 710.8 | 12.70 |
| 837 | R.GIDKQGALLLEQDGVIKPWMGGEISLR.S | | 731.648 | 4 | 2923.571 | 2923.571 | 0 | 0.0 | 5513 | 701.0 | 701.0 | 701.0 | 12.70 |
| 837 | R.GIDKQGALLLEQDGVIKPWMGGEISLR.S | | 731.649 | 4 | 2923.574 | 2923.571 | 0 | 1.0 | 5513 | 695.1 | 695.1 | 695.1 | 11.70 |
| 1612 | R.GIDKQGALLLEQDGVIKPWM[+15.995]GGEISLR.S | M[+16] | 735.647 | 4 | 2939.567 | 2939.566 | 0 | 0.2 | 5513 | 687.5 | 687.5 | 687.5 | 11.70 |
| 837 | R.GIDKQGALLLEQDGVIKPWMGGEISLR.S | | 731.649 | 4 | 2923.574 | 2923.571 | 0 | 0.9 | 5513 | 684.5 | 684.5 | 684.5 | 11.70 |
| 837 | R.GIDKQGALLLEQDGVIKPWMGGEISLR.S | | 731.649 | 4 | 2923.572 | 2923.571 | 0 | 0.3 | 5513 | 683.9 | 683.9 | 683.9 | 11.70 |
| 837 | R.GIDKQGALLLEQDGVIKPWMGGEISLR.S | | 731.649 | 4 | 2923.572 | 2923.571 | 0 | 0.3 | 5513 | 682.1 | 682.1 | 682.1 | 11.70 |
| 837 | R.GIDKQGALLLEQDGVIKPWMGGEISLR.S | | 975.195 | 3 | 2923.571 | 2923.571 | 0 | −0.1 | 5513 | 671.7 | 671.7 | 671.7 | 11.70 |
| 837 | R.GIDKQGALLLEQDGVIKPWMGGEISLR.S | | 731.648 | 4 | 2923.571 | 2923.571 | 0 | 0.1 | 5513 | 658.3 | 658.3 | 658.3 | 11.70 |
| 837 | R.GIDKQGALLLEQDGVIKPWMGGEISLR.S | | 731.648 | 4 | 2923.571 | 2923.571 | 0 | −0.2 | 5513 | 658.1 | 658.1 | 658.1 | 11.70 |
| 837 | R.GIDKQGALLLEQDGVIKPWMGGEISLR.S | | 731.648 | 4 | 2923.572 | 2923.571 | 0 | 0.2 | 5513 | 657.8 | 657.8 | 657.8 | 11.70 |
| 837 | R.GIDKQGALLLEQDGVIKPWMGGEISLR.S | | 731.648 | 4 | 2923.572 | 2923.571 | 0 | 0.2 | 5513 | 657.7 | 657.7 | 657.7 | 11.70 |
| 837 | R.GIDKQGALLLEQDGVIKPWMGGEISLR.S | | 731.648 | 4 | 2923.571 | 2923.571 | 0 | 0.0 | 5513 | 654.8 | 654.8 | 654.8 | 11.70 |
| 837 | R.GIDKQGALLLEQDGVIKPWMGGEISLR.S | | 975.195 | 3 | 2923.570 | 2923.571 | 0 | −0.3 | 5513 | 698.8 | 698.8 | 698.8 | 11.12 |
| 1612 | R.GIDKQGALLLEQDGVIKPWM[+15.995]GGEISLR.SM[+16] | | 735.647 | 4 | 2939.564 | 2939.566 | 0 | −0.6 | 5513 | 659.6 | 659.6 | 659.6 | 11.12 |
| 837 | R.GIDKQGALLLEQDGVIKPWMGGEISLR.S | | 731.648 | 4 | 2923.568 | 2923.571 | 0 | −1.0 | 5513 | 654.0 | 654.0 | 654.0 | 11.12 |
| 837 | R.GIDKQGALLLEQDGVIKPWMGGEISLR.S | | 975.196 | 3 | 2923.572 | 2923.571 | 0 | 0.4 | 5513 | 649.9 | 649.9 | 649.9 | 10.70 |
| 1612 | R.GIDKQGALLLEQDGVIKPWM[+15.995]GGEISLR.S | M[+16] | 735.647 | 4 | 2939.568 | 2939.566 | 0 | 0.7 | 5513 | 649.4 | 649.4 | 649.4 | 10.70 |
| 837 | R.GIDKQGALLLEQDGVIKPWMGGEISLR.S | | 731.648 | 4 | 2923.571 | 2923.571 | 0 | −0.1 | 5513 | 648.3 | 648.3 | 648.3 | 10.70 |
| 837 | R.GIDKQGALLLEQDGVIKPWMGGEISLR.S | | 731.649 | 4 | 2923.574 | 2923.571 | 0 | 0.8 | 5513 | 644.5 | 644.5 | 644.5 | 10.70 |
| 837 | R.GIDKQGALLLEQDGVIKPWMGGEISLR.S | | 731.648 | 4 | 2923.571 | 2923.571 | 0 | 0.1 | 5513 | 641.7 | 641.7 | 641.7 | 10.70 |
| 837 | R.GIDKQGALLLEQDGVIKPWMGGEISLR.S | | 731.649 | 4 | 2923.573 | 2923.571 | 0 | 0.8 | 5513 | 639.1 | 639.1 | 639.1 | 10.70 |
| 837 | R.GIDKQGALLLEQDGVIKPWMGGEISLR.S | | 731.649 | 4 | 2923.572 | 2923.571 | 0 | 0.3 | 5513 | 632.3 | 632.3 | 632.3 | 10.70 |
| 837 | R.GIDKQGALLLEQDGVIKPWMGGEISLR.S | | 731.899 | 4 | 2924.575 | 2923.571 | 1 | 0.4 | 5513 | 631.7 | 631.7 | 631.7 | 10.70 |
| 837 | R.GIDKQGALLLEQDGVIKPWMGGEISLR.S | | 731.899 | 4 | 2924.576 | 2923.571 | 1 | 0.5 | 5513 | 631.1 | 631.1 | 631.1 | 10.70 |
| 837 | R.GIDKQGALLLEQDGVIKPWMGGEISLR.S | | 731.649 | 4 | 2923.574 | 2923.571 | 0 | 1.0 | 5513 | 627.7 | 627.7 | 627.7 | 10.70 |
| 837 | R.GIDKQGALLLEQDGVIKPWMGGEISLR.S | | 731.649 | 4 | 2923.573 | 2923.571 | 0 | 0.5 | 5513 | 622.8 | 622.8 | 622.8 | 10.70 |
| 837 | R.GIDKQGALLLEQDGVIKPWMGGEISLR.S | | 731.649 | 4 | 2923.574 | 2923.571 | 0 | 1.0 | 5513 | 619.4 | 619.4 | 619.4 | 10.70 |
| 837 | R.GIDKQGALLLEQDGVIKPWMGGEISLR.S | | 731.649 | 4 | 2923.573 | 2923.571 | 0 | 0.5 | 5513 | 616.3 | 616.3 | 616.3 | 10.70 |
| 837 | R.GIDKQGALLLEQDGVIKPWMGGEISLR.S | | 731.649 | 4 | 2923.573 | 2923.571 | 0 | 0.7 | 5513 | 613.5 | 613.5 | 613.5 | 10.70 |
| 837 | R.GIDKQGALLLEQDGVIKPWMGGEISLR.S | | 731.649 | 4 | 2923.573 | 2923.571 | 0 | 0.6 | 5513 | 604.6 | 604.6 | 604.6 | 10.70 |
| 837 | R.GIDKQGALLLEQDGVIKPWMGGEISLR.S | | 1462.288 | 2 | 2923.569 | 2923.571 | 0 | −0.8 | 5513 | 670.6 | 670.6 | 670.6 | 9.70 |
| 837 | R.GIDKQGALLLEQDGVIKPWMGGEISLR.S | | 731.649 | 4 | 2923.573 | 2923.571 | 0 | 0.8 | 5513 | 596.6 | 596.6 | 596.6 | 9.70 |
| 837 | R.GIDKQGALLLEQDGVIKPWMGGEISLR.S | | 731.649 | 4 | 2923.573 | 2923.571 | 0 | 0.8 | 5513 | 593.6 | 593.6 | 593.6 | 9.70 |
| 837 | R.GIDKQGALLLEQDGVIKPWMGGEISLR.S | | 731.649 | 4 | 2923.574 | 2923.571 | 0 | 1.1 | 5513 | 588.5 | 588.5 | 588.5 | 9.70 |
| 837 | R.GIDKQGALLLEQDGVIKPWMGGEISLR.S | | 731.648 | 4 | 2923.571 | 2923.571 | 0 | 0.1 | 5513 | 577.6 | 577.6 | 577.6 | 9.70 |
| 1612 | R.GIDKQGALLLEQDGVIKPWM[+15.995]GGEISLR.S | M[+16] | 735.647 | 4 | 2939.568 | 2939.566 | 0 | 0.6 | 5513 | 577.6 | 577.6 | 577.6 | 9.70 |
| 837 | R.GIDKQGALLLEQDGVIKPWMGGEISLR.S | | 731.649 | 4 | 2923.574 | 2923.571 | 0 | 0.9 | 5513 | 571.5 | 571.5 | 571.5 | 9.70 |
| 837 | R.GIDKQGALLLEQDGVIKPWMGGEISLR.S | | 731.649 | 4 | 2923.572 | 2923.571 | 0 | 0.4 | 5513 | 559.7 | 559.7 | 559.7 | 8.97 |
| 837 | R.GIDKQGALLLEQDGVIKPWMGGEISLR.S | | 731.648 | 4 | 2923.571 | 2923.571 | 0 | 0.1 | 5513 | 554.8 | 554.8 | 554.8 | 8.97 |
| 837 | R.GIDKQGALLLEQDGVIKPWMGGEISLR.S | | 731.649 | 4 | 2923.572 | 2923.571 | 0 | 0.4 | 5513 | 553.6 | 553.6 | 553.6 | 8.97 |
| 837 | R.GIDKQGALLLEQDGVIKPWMGGEISLR.S | | 731.648 | 4 | 2923.571 | 2923.571 | 0 | 0.0 | 5513 | 546.6 | 546.6 | 546.6 | 8.97 |
| 837 | R.GIDKQGALLLEQDGVIKPWMGGEISLR.S | | 731.649 | 4 | 2923.573 | 2923.571 | 0 | 0.5 | 5513 | 530.9 | 530.9 | 530.9 | 8.97 |
| 1612 | R.GIDKQGALLLEQDGVIKPWM[+15.995]GGEISLR.S | M[+16] | 735.647 | 4 | 2939.567 | 2939.566 | 0 | 0.3 | 5513 | 521.9 | 521.9 | 521.9 | 8.97 |
| 837 | R.GIDKQGALLLEQDGVIKPWMGGEISLR.S | | 731.649 | 4 | 2923.574 | 2923.571 | 0 | 1.0 | 5513 | 519.7 | 519.7 | 519.7 | 8.79 |
| 1612 | R.GIDKQGALLLEQDGVIKPWM[+15.995]GGEISLR.S | M[+16] | 980.527 | 3 | 2939.566 | 2939.566 | 0 | 0.1 | 5513 | 514.2 | 514.2 | 514.2 | 8.79 |
| 837 | R.GIDKQGALLLEQDGVIKPWMGGEISLR.S | | 731.649 | 4 | 2923.572 | 2923.571 | 0 | 0.4 | 5513 | 485.9 | 485.9 | 485.9 | 8.79 |
| 837 | R.GIDKQGALLLEQDGVIKPWMGGEISLR.S | | 731.649 | 4 | 2923.572 | 2923.571 | 0 | 0.4 | 5513 | 522.0 | 522.0 | 522.0 | 8.38 |
| 837 | R.GIDKQGALLLEQDGVIKPWMGGEISLR.S | | 731.648 | 4 | 2923.572 | 2923.571 | 0 | 0.2 | 5513 | 516.1 | 516.1 | 516.1 | 8.20 |
| 837 | R.GIDKQGALLLEQDGVIKPWMGGEISLR.S | | 731.649 | 4 | 2923.574 | 2923.571 | 0 | 1.0 | 5513 | 509.6 | 509.6 | 509.6 | 8.20 |
| 837 | R.GIDKQGALLLEQDGVIKPWMGGEISLR.S | | 731.649 | 4 | 2923.573 | 2923.571 | 0 | 0.5 | 5513 | 481.0 | 481.0 | 481.0 | 8.20 |
| 837 | R.GIDKQGALLLEQDGVIKPWMGGEISLR.S | | 975.196 | 3 | 2923.574 | 2923.571 | 0 | 0.9 | 5513 | 554.8 | 554.8 | 554.8 | 8.09 |
| 837 | R.GIDKQGALLLEQDGVIKPWMGGEISLR.S | | 975.195 | 3 | 2923.571 | 2923.571 | 0 | −0.1 | 5513 | 512.5 | 512.5 | 512.5 | 7.91 |
| 837 | R.GIDKQGALLLEQDGVIKPWMGGEISLR.S | | 731.649 | 4 | 2923.573 | 2923.571 | 0 | 0.6 | 5513 | 486.9 | 486.9 | 486.9 | 7.91 |
| 837 | R.GIDKQGALLLEQDGVIKPWMGGEISLR.S | | 731.649 | 4 | 2923.574 | 2923.571 | 0 | 1.0 | 5513 | 436.5 | 436.5 | 436.5 | 7.81 |
| 837 | R.GIDKQGALLLEQDGVIKPWMGGEISLR.S | | 731.648 | 4 | 2923.572 | 2923.571 | 0 | 0.3 | 5513 | 444.4 | 365.0 | 365.0 | 7.75 |
| 1612 | R.GIDKQGALLLEQDGVIKPWM[+15.995]GGEISLR.S | M[+16] | 588.720 | 5 | 2939.568 | 2939.566 | 0 | 0.8 | 5513 | 463.2 | 463.2 | 463.2 | 7.52 |
| 837 | R.GIDKQGALLLEQDGVIKPWMGGEISLR.S | | 731.648 | 4 | 2923.571 | 2923.571 | 0 | −0.2 | 5513 | 438.0 | 438.0 | 438.0 | 7.52 |
| 837 | R.GIDKQGALLLEQDGVIKPWMGGEISLR.S | | 731.649 | 4 | 2923.572 | 2923.571 | 0 | 0.3 | 5513 | 431.5 | 431.5 | 431.5 | 7.52 |
| 837 | R.GIDKQGALLLEQDGVIKPWMGGEISLR.S | | 731.649 | 4 | 2923.573 | 2923.571 | 0 | 0.5 | 5513 | 373.2 | 373.2 | 373.2 | 7.40 |
| 837 | R.GIDKQGALLLEQDGVIKPWMGGEISLR.S | | 975.195 | 3 | 2923.571 | 2923.571 | 0 | 0.1 | 5513 | 434.8 | 434.8 | 434.8 | 7.33 |
| 837 | R.GIDKQGALLLEQDGVIKPWMGGEISLR.S | | 731.899 | 4 | 2924.572 | 2923.571 | 1 | 0.8 | 5513 | 488.7 | 488.7 | 488.7 | 7.32 |
| 837 | R.GIDKQGALLLEQDGVIKPWMGGEISLR.S | | 731.900 | 4 | 2924.578 | 2923.571 | 1 | 1.4 | 5513 | 378.8 | 378.8 | 378.8 | 7.22 |
| 837 | R.GIDKQGALLLEQDGVIKPWMGGEISLR.S | | 975.530 | 3 | 2924.577 | 2923.571 | 1 | 0.7 | 5513 | 360.8 | 360.8 | 360.8 | 7.22 |
| 837 | R.GIDKQGALLLEQDGVIKPWMGGEISLR.S | | 731.642 | 4 | 2923.545 | 2923.571 | 0 | 8.9 | 5513 | 534.8 | 534.8 | 534.8 | 6.81 |
| 837 | R.GIDKQGALLLEQDGVIKPWMGGEISLR.S | | 731.649 | 4 | 2923.574 | 2923.571 | 0 | 1.2 | 5513 | 357.1 | 357.1 | 357.1 | 5.70 |
| 837 | R.GIDKQGALLLEQDGVIKPWMGGEISLR.S | | 731.648 | 4 | 2923.571 | 2923.571 | 0 | 0.0 | 5513 | 328.9 | 328.9 | 328.9 | 5.70 |
| 837 | R.GIDKQGALLLEQDGVIKPWMGGEISLR.S | | 731.899 | 4 | 2924.574 | 2923.571 | 1 | 0.3 | 5513 | 311.1 | 311.1 | 311.1 | 4.50 |
| 1659 | K.QGALLLEQDGVIKPWM[+15.995]GGEISLR.S | M[+16] | 842.785 | 3 | 2526.339 | 2526.339 | 0 | 0.3 | 5517 | 792.1 | 792.1 | 792.1 | 13.98 |
| 915 | K.QGALLLEQDGVIKPWMGGEISLR.S | | 1256.677 | 2 | 2512.347 | 2510.344 | 2 | −1.3 | 5517 | 457.2 | 457.2 | 457.2 | 6.65 |
| 915 | K.QGALLLEQDGVIKPWMGGEISLR.S | | 837.452 | 3 | 2510.342 | 2510.344 | 0 | 0.7 | 5517 | 325.0 | 300.0 | 300.0 | 5.19 |
| 864 | R.SAEKLQLPPLER.L | | 690.896 | 2 | 1380.785 | 1380.785 | 0 | 0.1 | 5540 | 670.5 | 670.5 | 670.5 | 11.44 |
| 864 | R.SAEKLQLPPLER.L | | 690.896 | 2 | 1380.784 | 1380.785 | 0 | −0.3 | 5540 | 570.3 | 570.3 | 570.3 | 8.86 |
| 864 | R.SAEKLQLPPLER.L | | 690.896 | 2 | 1380.785 | 1380.785 | 0 | 0.1 | 5540 | 486.0 | 486.0 | 486.0 | 8.53 |
| 864 | R.SAEKLQLPPLER.L | | 690.896 | 2 | 1380.784 | 1380.785 | 0 | −0.3 | 5540 | 599.4 | 599.4 | 599.4 | 8.26 |
| 864 | R.SAEKLQLPPLER.L | | 460.933 | 3 | 1380.785 | 1380.785 | 0 | 0.1 | 5540 | 475.5 | 475.5 | 475.5 | 7.78 |
| 864 | R.SAEKLQLPPLER.L | | 460.933 | 3 | 1380.784 | 1380.785 | 0 | −0.1 | 5540 | 448.6 | 448.6 | 448.6 | 7.78 |
| 864 | R.SAEKLQLPPLER.L | | 460.933 | 3 | 1380.785 | 1380.785 | 0 | 0.0 | 5540 | 482.8 | 482.8 | 482.8 | 7.64 |
| 864 | R.SAEKLQLPPLER.L | | 460.933 | 3 | 1380.785 | 1380.785 | 0 | 0.2 | 5540 | 435.5 | 435.5 | 435.5 | 7.55 |
| 4650 | R.SAEKLQLPPLERLTLD.- | | 912.017 | 2 | 1823.027 | 1823.027 | 0 | 0.1 | 5540 | 420.5 | 420.5 | 420.5 | 7.20 |
| 864 | R.SAEKLQLPPLER.L | | 460.933 | 3 | 1380.784 | 1380.785 | 0 | −0.2 | 5540 | 399.6 | 399.6 | 399.6 | 7.14 |
| 4650 | R.SAEKLQLPPLERLTLD.- | | 608.347 | 3 | 1823.027 | 1823.027 | 0 | 0.0 | 5540 | 379.8 | 379.8 | 379.8 | 6.80 |
| 864 | R.SAEKLQLPPLER.L | | 460.933 | 3 | 1380.784 | 1380.785 | 0 | −0.2 | 5540 | 386.8 | 386.8 | 386.8 | 6.56 |
| 1905 | K.LQLPPLER.L | | 483.293 | 2 | 965.578 | 965.578 | 0 | −0.1 | 5544 | 417.5 | 417.5 | 417.5 | 7.35 |
| 1905 | K.LQLPPLER.L | | 483.293 | 2 | 965.578 | 965.578 | 0 | 0.2 | 5544 | 413.8 | 413.8 | 413.8 | 7.35 |
| 1905 | K.LQLPPLER.L | | 483.293 | 2 | 965.578 | 965.578 | 0 | 0.0 | 5544 | 398.8 | 398.8 | 398.8 | 7.23 |

TABLE 9
RNF213 Peptides Rep 3

| | | | | | | | | | Start- | | | | |
|---|---|---|---|---|---|---|---|---|---|---|---|---|---|
| SEQ | | Modifi- | | | | Calc. | Off- | Mass | ing | | | | |
| ID | Peptide | cation | Observed | | Observed | mass | by-x | error | posi- | | | Delta | \|Log |
| NO: | <ProteinMetrics Confidential> | Type(s) | m/z | Z | (M + H) | (M + H) | error | (ppm) | tion | Score | Delta | Mod | Prob\| |
| 880 | K.ASWTVQESK.K | | 518.259 | 2 | 1035.510 | 1035.511 | 0 | −0.1 | 81 | 501.6 | 501.6 | 501.6 | 8.01 |
| 880 | K.ASWTVQESK.K | | 518.259 | 2 | 1035.510 | 1035.511 | 0 | 0.4 | 81 | 426.4 | 426.4 | 426.4 | 7.44 |
| 884 | R.GLSQEGTGPPTSAGEGHSR.T | | 608.954 | 3 | 1824.849 | 1824.847 | 0 | 0.9 | 215 | 842.0 | 842.0 | 842.0 | 14.78 |
| 884 | R.GLSQEGTGPPTSAGEGHSR.T | | 608.954 | 3 | 1824.848 | 1824.847 | 0 | 0.3 | 215 | 736.0 | 736.0 | 736.0 | 12.79 |
| 884 | R.GLSQEGTGPPTSAGEGHSR.T | | 608.954 | 3 | 1824.847 | 1824.847 | 0 | −0.2 | 215 | 700.1 | 700.1 | 700.1 | 12.79 |
| 884 | R.GLSQEGTGPPTSAGEGHSR.T | | 912.927 | 2 | 1824.848 | 1824.847 | 0 | 0.2 | 215 | 684.5 | 684.5 | 684.5 | 11.79 |
| 884 | R.GLSQEGTGPPTSAGEGHSR.T | | 608.955 | 3 | 1824.849 | 1824.847 | 0 | 1.0 | 215 | 662.6 | 662.6 | 662.6 | 11.79 |
| 884 | R.GLSQEGTGPPTSAGEGHSR.T | | 608.954 | 3 | 1824.846 | 1824.847 | 0 | −0.4 | 215 | 655.5 | 655.5 | 655.5 | 11.79 |
| 1079 | R.GLSQEGTGPPTSAGEGHSRTEDAAQELLLPESK.G | | 838.160 | 4 | 3349.616 | 3349.614 | 0 | 0.7 | 215 | 659.5 | 659.5 | 659.5 | 10.85 |
| 1079 | R.GLSQEGTGPPTSAGEGHSRTEDAAQELLLPESK.G | | 1117.210 | 3 | 3349.614 | 3349.614 | 0 | 0.1 | 215 | 654.9 | 654.9 | 654.9 | 10.85 |
| 884 | R.GLSQEGTGPPTSAGEGHSR.T | | 456.967 | 4 | 1824.847 | 1824.847 | 0 | −0.1 | 215 | 562.8 | 516.7 | 516.7 | 9.25 |
| 1079 | R.GLSQEGTGPPTSAGEGHSRTEDAAQELLLPESK.G | | 838.159 | 4 | 3349.614 | 3349.614 | 0 | −0.2 | 215 | 596.8 | 596.8 | 596.8 | 8.85 |
| 1467 | R.GLSQEGTGPPTSAGEGHS[+79.966]RTEDAAQELLLPESK.G | S[+80] | 858.401 | 4 | 3430.583 | 3429.581 | 1 | −0.3 | 215 | 588.3 | 588.3 | 22.6 | 8.31 |
| 1079 | R.GLSQEGTGPPTSAGEGHSRTEDAAQELLLPESK.G | | 838.159 | 4 | 3349.613 | 3349.614 | 0 | −0.4 | 215 | 506.4 | 506.4 | 506.4 | 8.05 |
| 1079 | R.GLSQEGTGPPTSAGEGHSRTEDAAQELLLPESK.G | | 1117.209 | 3 | 3349.613 | 3349.614 | 0 | −0.5 | 215 | 568.7 | 568.7 | 568.7 | 7.94 |
| 884 | R.GLSQEGTGPPTSAGEGHSR.T | | 608.954 | 3 | 1824.847 | 1824.847 | 0 | −0.1 | 215 | 361.6 | 361.6 | 361.6 | 7.73 |
| 1079 | R.GLSQEGTGPPTSAGEGHSRTEDAAQELLLPESK.G | | 670.729 | 5 | 3349.616 | 3349.614 | 0 | 0.5 | 215 | 517.4 | 517.4 | 517.4 | 6.74 |
| 884 | R.GLSQEGTGPPTSAGEGHSR.T | | 609.282 | 3 | 1825.831 | 1824.847 | 1 | −10.4 | 215 | 496.2 | 496.2 | 496.2 | 6.68 |
| 1079 | R.GLSQEGTGPPTSAGEGHSRTEDAAQELLLPESK.G | | 670.729 | 5 | 3349.616 | 3349.614 | 0 | 0.4 | 215 | 459.8 | 459.8 | 459.8 | 6.53 |
| 884 | R.GLSQEGTGPPTSAGEGHSR.T | | 608.954 | 3 | 1824.848 | 1824.847 | 0 | 0.4 | 215 | 346.7 | 346.7 | 346.7 | 6.45 |
| 1079 | R.GLSQEGTGPPTSAGEGHSRTEDAAQELLLPESK.G | | 670.729 | 5 | 3349.616 | 3349.614 | 0 | 0.7 | 215 | 371.2 | 371.2 | 371.2 | 6.06 |
| 1079 | R.GLSQEGTGPPTSAGEGHSRTEDAAQELLLPESK.G | | 838.158 | 4 | 3349.609 | 3349.614 | 0 | −1.5 | 215 | 365.1 | 365.1 | 365.1 | 5.88 |
| 1079 | R.GLSQEGTGPPTSAGEGHSRTEDAAQELLLPESK.G | | 670.729 | 5 | 3349.616 | 3349.614 | 0 | 0.5 | 215 | 400.3 | 345.0 | 345.0 | 4.99 |
| 842 | R.GLSQEGTGPPTSAGEGHSRTEDAAQELLLPESKGGSSEPGTELQ | | 970.894 | 7 | 6790.217 | 6790.214 | 0 | 0.3 | 215 | 511.1 | 511.1 | 511.1 | 4.91 |
| | TTEQQAGASASMAVDAVAEPANAVK.G | | | | | | | | | | | | |
| 1185 | R.TEDAAQELLLPESK.G | | 772.396 | 2 | 1543.785 | 1543.785 | 0 | 0.1 | 234 | 647.8 | 590.0 | 590.0 | 10.58 |
| 2915 | R.TEDAAQELLLPESKGGSSEPGTELQTTEQQAGASASMAVDAV | | 1246.851 | 4 | 4984.382 | 4984.385 | 0 | −0.6 | 234 | 745.7 | 745.7 | 745.7 | 10.01 |
| | AEPANAVK.G | | | | | | | | | | | | |
| 1185 | R.TEDAAQELLLPESK.G | | 772.396 | 2 | 1543.785 | 1543.785 | 0 | 0.2 | 234 | 582.0 | 534.1 | 534.1 | 9.58 |
| 1185 | R.TEDAAQELLLPESK.G | | 772.396 | 2 | 1543.785 | 1543.785 | 0 | −0.1 | 234 | 468.4 | 326.1 | 326.1 | 7.40 |
| 2915 | R.TEDAAQELLLPESKGGSSEPGTELQTTEQQAGASASMAVDAV | | 997.883 | 5 | 4985.387 | 4984.385 | 1 | −0.4 | 234 | 556.5 | 556.5 | 305.8 | 6.29 |
| | AEPANAVK.G | | | | | | | | | | | | |
| 1185 | R.TEDAAQELLLPESK.G | | 515.267 | 3 | 1543.786 | 1543.785 | 0 | 0.4 | 234 | 385.5 | 282.8 | 282.8 | 5.43 |
| 2915 | R.TEDAAQELLLPESKGGSSEPGTELQTTEQQAGASASMAVDAV | | 997.681 | 5 | 4984.377 | 4984.385 | 0 | −1.6 | 234 | 513.8 | 513.8 | 513.8 | 4.78 |
| | AEPANAVK.G | | | | | | | | | | | | |
| 1078 | K.GGSSEPGTELQTTEQQAGASASMAVDAVAEPANAVKGAGK.E | | 1258.269 | 3 | 3772.793 | 3772.793 | 0 | −0.1 | 248 | 949.8 | 949.8 | 949.8 | 15.95 |
| 1078 | K.GGSSEPGTELQTTEQQAGASASMAVDAVAEPANAVKGAGK.E | | 943.955 | 4 | 3772.797 | 3772.793 | 0 | 1.2 | 248 | 917.6 | 917.6 | 917.6 | 15.35 |
| 1078 | K.GGSSEPGTELQTTEQQAGASASMAVDAVAEPANAVKGAGK.E | | 1258.603 | 3 | 3773.794 | 3772.793 | 1 | −0.5 | 248 | 904.3 | 904.3 | 638.2 | 14.95 |
| 1078 | K.GGSSEPGTELQTTEQQAGASASMAVDAVAEPANAVKGAGK.E | | 1258.269 | 3 | 3772.793 | 3772.793 | 0 | −0.1 | 248 | 895.4 | 895.4 | 895.4 | 14.84 |
| 1078 | K.GGSSEPGTELQTTEQQAGASASMAVDAVAEPANAVKGAGK.E | | 943.954 | 4 | 3772.793 | 3772.793 | 0 | 0.1 | 248 | 771.0 | 771.0 | 771.0 | 12.32 |
| 1210 | K.GGSSEPGTELQTTEQQAGASASMAVDAVAEPANAVKGAGKE | | 1040.998 | 4 | 4160.969 | 4160.971 | 0 | −0.5 | 248 | 845.3 | 796.8 | 796.8 | 12.22 |
| | MK.E | | | | | | | | | | | | |
| 1210 | K.GGSSEPGTELQTTEQQAGASASMAVDAVAEPANAVKGAGKE | | 1040.998 | 4 | 4160.971 | 4160.971 | 0 | −0.1 | 248 | 784.9 | 725.5 | 725.5 | 12.13 |
| | MK.E | | | | | | | | | | | | |
| 1066 | K.GGSSEPGTELQTTEQQAGASASMAVDAVAEPANAVK.G | | 1154.213 | 3 | 3460.623 | 3459.618 | 1 | 0.5 | 248 | 707.8 | 585.5 | 308.2 | 11.58 |
| 1567 | K.GGSSEPGTELQTTEQQAGASASM[+15.995]AVDAVAEPANAVK | M[+16] | 1159.211 | 3 | 3475.617 | 3475.613 | 0 | 1.2 | 248 | 703.9 | 703.9 | 703.9 | 11.58 |
| | .G | | | | | | | | | | | | |
| 1483 | K.GGSSEPGTELQTTEQQAGASASMAVDAVAEPAN[+0.984]AVK | N[+1] | 1041.497 | 4 | 4162.964 | 4161.955 | 1 | 1.4 | 248 | 738.3 | 738.3 | 206.2 | 11.13 |
| | GAGKEMK.E | | | | | | | | | | | | |
| 1066 | K.GGSSEPGTELQTTEQQAGASASMAVDAVAEPANAVK.G | | 865.661 | 4 | 3459.621 | 3459.618 | 0 | 0.8 | 248 | 681.9 | 681.9 | 681.9 | 10.58 |
| 1567 | K.GGSSEPGTELQTTEQQAGASASM[+15.995]AVDAVAEPANAVK | M[+16] | 1159.210 | 3 | 3475.615 | 3475.613 | 0 | 0.5 | 248 | 669.1 | 669.1 | 669.1 | 10.58 |
| | .G | | | | | | | | | | | | |
| 1066 | K.GGSSEPGTELQTTEQQAGASASMAVDAVAEPANAVK.G | | 1153.877 | 3 | 3459.617 | 3459.618 | 0 | −0.3 | 248 | 651.4 | 651.4 | 651.4 | 10.58 |
| 1066 | K.GGSSEPGTELQTTEQQAGASASMAVDAVAEPANAVK.G | | 865.660 | 4 | 3459.620 | 3459.618 | 0 | 0.4 | 248 | 637.0 | 637.0 | 637.0 | 9.58 |
| 1066 | K.GGSSEPGTELQTTEQQAGASASMAVDAVAEPANAVK.G | | 865.660 | 4 | 3459.619 | 3459.618 | 0 | 0.3 | 248 | 604.4 | 604.4 | 604.4 | 9.58 |
| 1210 | K.GGSSEPGTELQTTEQQAGASASMAVDAVAEPANAVKGAGKE | | 833.202 | 5 | 4161.979 | 4160.971 | 1 | 1.0 | 248 | 671.7 | 671.7 | 231.8 | 9.04 |
| | MK.E | | | | | | | | | | | | |
| 1066 | K.GGSSEPGTELQTTEQQAGASASMAVDAVAEPANAVK.G | | 865.660 | 4 | 3459.618 | 3459.618 | 0 | −0.1 | 248 | 622.5 | 622.5 | 622.5 | 9.03 |
| 1567 | K.GGSSEPGTELQTTEQQAGASASM[+15.995]AVDAVAEPANAVK | M[+16] | 869.659 | 4 | 3475.613 | 3475.613 | 0 | 0.0 | 248 | 600.4 | 600.4 | 600.4 | 9.03 |
| | .G | | | | | | | | | | | | |
| 1078 | K.GGSSEPGTELQTTEQQAGASASMAVDAVAEPANAVKGAGK.E | | 755.365 | 5 | 3772.794 | 3772.793 | 0 | 0.3 | 248 | 611.6 | 604.2 | 604.2 | 8.77 |
| 1567 | K.GGSSEPGTELQTTEQQAGASASM[+15.995]AVDAVAEPANAVK | M[+16] | 1159.542 | 3 | 3476.612 | 3475.613 | 1 | −1.1 | 248 | 641.7 | 641.7 | 529.2 | 8.66 |
| | .G | | | | | | | | | | | | |
| 1066 | K.GGSSEPGTELQTTEQQAGASASMAVDAVAEPANAVK.G | | 1153.878 | 3 | 3459.620 | 3459.618 | 0 | 0.6 | 248 | 592.6 | 592.6 | 592.6 | 8.58 |
| 1066 | K.GGSSEPGTELQTTEQQAGASASMAVDAVAEPANAVK.G | | 1153.879 | 3 | 3459.623 | 3459.618 | 0 | 1.4 | 248 | 540.2 | 540.2 | 540.2 | 7.90 |
| 1066 | K.GGSSEPGTELQTTEQQAGASASMAVDAVAEPANAVK.G | | 1153.877 | 3 | 3459.617 | 3459.618 | 0 | −0.3 | 248 | 536.0 | 536.0 | 536.0 | 7.35 |
| 1210 | K.GGSSEPGTELQTTEQQAGASASMAVDAVAEPANAVKGAGKE | | 694.335 | 6 | 4160.972 | 4160.971 | 0 | 0.2 | 248 | 569.4 | 485.9 | 485.9 | 7.04 |
| | MK.E | | | | | | | | | | | | |
| 1066 | K.GGSSEPGTELQTTEQQAGASASMAVDAVAEPANAVK.G | | 865.660 | 4 | 3459.618 | 3459.618 | 0 | 0.0 | 248 | 510.4 | 510.4 | 510.4 | 7.00 |
| 1567 | K.GGSSEPGTELQTTEQQAGASASM[+15.995]AVDAVAEPANAVK | M[+16] | 1159.212 | 3 | 3475.620 | 3475.613 | 0 | 2.1 | 248 | 599.2 | 599.2 | 599.2 | 6.51 |
| | .G | | | | | | | | | | | | |
| 1066 | K.GGSSEPGTELQTTEQQAGASASMAVDAVAEPANAVK.G | | 1153.876 | 3 | 3459.612 | 3459.618 | 0 | −1.6 | 248 | 482.6 | 482.6 | 482.6 | 5.88 |
| 1210 | K.GGSSEPGTELQTTEQQAGASASMAVDAVAEPANAVKGAGKE | | 1040.993 | 4 | 4160.951 | 4160.971 | 0 | −4.9 | 248 | 490.4 | 490.4 | 490.4 | 4.91 |
| | MK.E | | | | | | | | | | | | |
| 1483 | K.GGSSEPGTELQTTEQQAGASASMAVDAVAEPAN[+0.984]AVK | N[+1] | 1041.495 | 4 | 4162.956 | 4161.955 | 1 | −0.5 | 248 | 349.6 | 349.6 | 42.4 | 3.21 |
| | GAGKEMK.E | | | | | | | | | | | | |
| 1725 | R.MKQPPATTPPFKTHC[+57.021]QEAETK.T | C[+57] | 607.550 | 4 | 2427.178 | 2427.180 | 0 | −0.7 | 296 | 679.7 | 679.7 | 679.7 | 10.28 |
| 1725 | R.MKQPPATTPPFKTHC[+57.021]QEAETK.T | C[+57] | 607.551 | 1 | 2427.181 | 2427.180 | 0 | 0.8 | 296 | 602.1 | 602.1 | 602.1 | 10.19 |
| 1666 | R.M[+15.995]KQPPATTPPFKTHC[+57.021]QEAETK.T | C[+57], | 611.549 | 4 | 2443.175 | 2443.174 | 0 | 0.3 | 296 | 562.1 | 562.1 | 562.1 | 9.19 |
| | | M[+16] | | | | | | | | | | | |
| 1725 | R.MKQPPATTPPFKTHC[+57.021]QEAETK.T | C[+57] | 809.731 | 3 | 2427.179 | 2427.180 | 0 | −0.1 | 296 | 1619.2 | 619.2 | 619.2 | 8.85 |
| 1666 | R.M[+15.995]KQPPATTPPFKTHC[+57.021]QEAETK.T | C[+57], | 611.549 | 4 | 2443.175 | 2443.174 | 0 | 0.0 | 296 | 506.3 | 506.3 | 506.3 | 8.39 |
| | | M[+16] | | | | | | | | | | | |
| 1725 | R.MKQPPATTPPFKTHC[+57.021]QEAETK.T | C[+57] | 486.242 | 5 | 2427.179 | 2427.180 | 0 | −0.3 | 296 | 446.5 | 446.5 | 446.5 | 8.18 |
| 1666 | R.M[+15.995]KQPPATTPPFKTHC[+57.021]QEAETK.T | C[+57], | 489.441 | 5 | 2443.174 | 2443.174 | 0 | −0.1 | 296 | 440.3 | 440.3 | 440.3 | 8.18 |
| | | M[+16] | | | | | | | | | | | |
| 824 | R.MKQPPATTPPFK.T | | 448.244 | 3 | 1342.719 | 1342.719 | 0 | 0.0 | 296 | 436.7 | 436.7 | 436.7 | 7.60 |
| 1662 | R.MKQPPATT[+79.966]PPFKTHC[+57.021]QEAETK.T | C[+57], | 627.542 | 4 | 2507.147 | 2507.146 | 0 | 0.3 | 296 | 424.7 | 406.6 | 32.7 | 7.47 |
| | | T[+80] | | | | | | | | | | | |
| 1725 | R.MKQPPATTPPFKTHC[+57.021]QEAETK.T | C[+57] | 486.242 | 5 | 2427.179 | 2427.180 | 0 | 0.0 | 296 | 420.5 | 420.5 | 420.5 | 7.47 |
| 1725 | R.MKQPPATTPPFKTHC[+57.021]QEAETK.T | C[+57] | 486.242 | 5 | 2427.180 | 2427.180 | 0 | 0.2 | 296 | 446.2 | 446.2 | 446.2 | 7.41 |
| 824 | R.MKQPPATTPPFK.T | | 448.245 | 3 | 1342.719 | 1342.719 | 0 | 0.2 | 296 | 417.2 | 417.2 | 417.2 | 7.38 |
| 1666 | R.M[+15.995]KQPPATTPPFKTHC[+57.021]QEAETK.T | C[+57], | 489.441 | 5 | 2443.174 | 2443.174 | 0 | −0.1 | 296 | 393.2 | 393.2 | 393.2 | 7.36 |
| | | M[+16] | | | | | | | | | | | |
| 1666 | R.M[+15.995]KQPPATTPPFKTHC[+57.021]QEAETK.T | C[+57], | 489.441 | 5 | 2443.177 | 2443.174 | 0 | 1.0 | 296 | 377.1 | 377.1 | 377.1 | 7.36 |
| | | M[+16] | | | | | | | | | | | |
| 824 | R.MKQPPATTPPFK.T | | 671.863 | 2 | 1342.718 | 1342.719 | 0 | −0.8 | 296 | 395.6 | 395.6 | 395.6 | 6.35 |
| 1666 | R.M[+15.995]KQPPATTPPFKTHC[+57.021]QEAETK.T | C[+57], | 611.551 | 4 | 2443.181 | 2443.174 | 0 | 2.6 | 296 | 477.9 | 477.9 | 477.9 | 6.34 |
| | | M[+16] | | | | | | | | | | | |
| 824 | R.MKQPPATTPPFK.T | | 671.863 | 2 | 1342.719 | 1342.719 | 0 | 0.5 | 296 | 347.0 | 347.0 | 347.0 | 5.78 |
| 1539 | R.M[+15.995]KQPPATTPPFK.T | M[+16] | 453.576 | 3 | 1358.713 | 1358.714 | 0 | −0.3 | 296 | 352.9 | 259.5 | 259.5 | 5.48 |
| 824 | R.MKQPPATTPPFK.T | | 448.244 | 3 | 1342.719 | 1342.719 | 0 | −0.1 | 296 | 310.6 | 310.6 | 310.6 | 5.32 |
| 1539 | R.M[+15.995]KQPPATTPPFK.T | M[+16] | 453.576 | 3 | 1358.712 | 1358.714 | 0 | −1.1 | 296 | 410.6 | 277.3 | 277.3 | 4.99 |
| 1666 | R.M[+15.995]KQPPATTPPFKTHC[+57.021]QEAETK.T | C[+57], | 489.440 | 5 | 2443.171 | 2443.174 | 0 | −1.5 | 296 | 344.1 | 344.1 | 344.1 | 4.73 |
| | | M[+16] | | | | | | | | | | | |
| 1687 | K.QPPATTPPFKTHC[+57.021]QEAETK.T | C[+57] | 542.767 | 4 | 2168.046 | 2168.044 | 0 | 0.9 | 298 | 360.3 | 360.3 | 360.3 | 7.70 |
| 3492 | K.QPPATTPPFK.T | | 542.295 | 2 | 1083.583 | 1083.583 | 0 | 0.0 | 298 | 331.2 | 331.2 | 331.2 | 5.86 |
| 1690 | K.THC[+57.021]QEAETKTKDEMAAAEEK.V | C[+57] | 769.681 | 3 | 2307.027 | 2307.023 | 0 | 2.0 | 308 | 817.3 | 817.3 | 817.3 | 11.55 |
| 1690 | K.THC[+57.021]QEAETKTKDEMAAAEEK.V | C[+57] | 577.511 | 4 | 2307.024 | 2307.023 | 0 | 0.4 | 308 | 608.6 | 608.6 | 608.6 | 10.19 |
| 1667 | K.THC[+57.021]QEAETKTKDEM[+15.995]AAAEEK.V | C[+57], | 581.510 | 4 | 2323.019 | 2323.018 | 0 | 0.4 | 308 | 512.1 | 512.1 | 512.1 | 7.84 |
| | | M[+16] | | | | | | | | | | | |
| 1690 | K.THC[+57.021]QEAETKTKDEMAAAEEK.V | C[+57] | 577.512 | 4 | 2307.024 | 2307.023 | 0 | 0.7 | 308 | 403.8 | 403.8 | 403.8 | 7.25 |
| 1690 | K.THC[+57.021]QEAETKTKDEMAAAEEK.V | C[+57] | 462.210 | 5 | 2307.022 | 2307.023 | 0 | −0.2 | 308 | 374.2 | 374.2 | 374.2 | 7.14 |
| 1357 | K.TKDEMAAAEEKVGK.N | | 502.921 | 3 | 1506.747 | 1506.747 | 0 | 0.0 | 317 | 696.0 | 617.6 | 617.6 | 10.99 |
| 1357 | K.TKDEMAAAEEKVGK.N | | 502.920 | 3 | 1506.747 | 1506.747 | 0 | −0.2 | 317 | 359.6 | 359.6 | 359.6 | 5.26 |
| 1493 | K.VGKN[+0.984]EQGEPEDLKKPEGK.N | N[+1] | 496.757 | 4 | 1984.008 | 1983.003 | 1 | 0.6 | 328 | 324.3 | 324.3 | 324.3 | 5.65 |
| 3402 | K.VGKNEQGEPEDLKKPEGK.N | | 496.256 | 4 | 1982.001 | 1982.019 | 0 | 8.9 | 328 | 333.8 | 333.8 | 333.8 | 4.09 |
| 3625 | R.SAAAVKNEKEQK.N | | 434.905 | 3 | 1302.701 | 1302.701 | 0 | −0.4 | 348 | 356.7 | 356.7 | 356.7 | 5.44 |
| 2203 | K.NQEADVQEVK.A | | 580.283 | 2 | 1159.559 | 1159.559 | 0 | −0.3 | 360 | 443.5 | 443.5 | 443.5 | 7.60 |
| 4142 | K.ASTLSPGGGVTVFFHAIISLHFPFNPDLHK.V | | 642.142 | 5 | 3206.683 | 3206.679 | 0 | 1.4 | 370 | 673.0 | 673.0 | 673.0 | 11.11 |
| 994 | K.SSLLGSGDWHQYYDIVYMKPHGR.L | | 678.078 | 4 | 2709.289 | 2709.288 | 0 | 0.3 | 484 | 531.3 | 531.3 | 531.3 | 9.12 |
| 994 | K.SSLLGSGDWHQYYDIVYMKPHGR.L | | 542.663 | 5 | 2709.288 | 2709.288 | 0 | 0.1 | 484 | 430.9 | 430.9 | 430.9 | 8.62 |
| 1590 | K.SSLLGSGDWHQYYDIVYM[+15.995]KPHGR.L | M[+16] | 682.076 | 4 | 2725.284 | 2725.283 | 0 | 0.3 | 484 | 495.6 | 495.6 | 495.6 | 18.21 |
| 994 | K.SSLLGSGDWHQYYDIVYMKPHGR.L | | 678.078 | 4 | 2709.289 | 2709.288 | 0 | 0.4 | 484 | 423.6 | 423.6 | 423.6 | 7.85 |
| 1590 | K.SSLLGSGDWHQYYDIVYM[+15.995]KPHGR.L | M[+16] | 682.077 | 4 | 2725.285 | 2725.283 | 0 | 0.8 | 484 | 433.2 | 433.2 | 433.2 | 7.65 |
| 860 | K.VMNHITDGPRKDLVK.G | | 574.982 | 3 | 1722.932 | 1722.932 | 0 | 0.1 | 510 | 577.8 | 577.8 | 577.8 | 7.64 |
| 860 | K.VMNHITDGPRKDLVK.G | | 431.489 | 4 | 1722.933 | 1722.932 | 0 | 0.5 | 510 | 366.1 | 366.1 | 366.1 | 6.93 |
| 860 | K.VMNHITDGPRKDLVK.G | | 431.489 | 4 | 1722.933 | 1722.932 | 0 | 0.4 | 510 | 362.6 | 362.6 | 362.6 | 6.93 |
| 860 | K.VMNHITDGPRKDLVK.G | | 861.970 | 2 | 1722.933 | 1722.932 | 0 | 0.3 | 510 | 482.3 | 482.3 | 482.3 | 6.28 |
| 860 | K.VMNHITDGPRKDLVK.G | | 431.489 | 4 | 1722.933 | 1722.932 | 0 | 0.4 | 510 | 332.6 | 332.6 | 332.6 | 5.64 |
| 1526 | K.VM[+15.995]NHITDGPRKDLVK.G | M[+16] | 580.314 | 3 | 1738.927 | 1738.927 | 0 | 0.0 | 510 | 374.6 | 374.6 | 374.6 | 5.38 |
| 1834 | K.RYLWQHLKK.H | | 424.584 | 3 | 1271.737 | 1271.737 | 0 | 0.1 | 588 | 405.1 | 405.1 | 405.1 | 5.84 |
| 2363 | R.YLWQHLK.K | | 494.274 | 2 | 987.541 | 987.541 | 0 | −0.2 | 589 | 448.4 | 448.4 | 448.4 | 7.42 |
| 907 | R.YLWQHLKK.H | | 558.322 | 2 | 1115.636 | 1115.636 | 0 | −0.1 | 589 | 483.6 | 483.6 | 483.6 | 6.40 |
| 2363 | R.YLWQHLK.K | | 494.275 | 2 | 987.542 | 987.541 | 0 | 0.7 | 589 | 310.6 | 310.6 | 310.6 | 5.58 |
| 907 | R.YLWQHLKK.H | | 558.322 | 2 | 1115.637 | 1115.636 | 0 | 0.4 | 589 | 357.7 | 357.7 | 357.7 | 4.25 |
| 956 | K.HVVPLPDGK.S | | 481.277 | 2 | 961.546 | 961.547 | 0 | −0.3 | 597 | 534.1 | 392.9 | 392.9 | 8.36 |
| 956 | K.HVVPLPDGK.S | | 481.277 | 2 | 961.546 | 961.547 | 0 | −0.4 | 597 | 463.5 | 383.4 | 383.4 | 6.89 |
| 956 | K.HVVPLPDGK.S | | 481.277 | 2 | 961.546 | 961.547 | 0 | −0.3 | 597 | 513.5 | 351.8 | 351.8 | 6.83 |
| 956 | K.HVVPLPDGK.S | | 481.277 | 2 | 961.546 | 961.547 | 0 | −0.3 | 597 | 375.9 | 216.4 | 216.4 | 5.89 |
| 1704 | K.STDFLPVDC[+57.021]PVR.S | C[+57] | 703.343 | 2 | 1405.678 | 1405.678 | 0 | 0.2 | 606 | 561.8 | 518.8 | 518.8 | 9.58 |
| 1704 | K.STDFLPVDC[+57.021]PVR.S | C[+57] | 469.231 | 3 | 1405.678 | 1405.678 | 0 | −0.1 | 606 | 443.2 | 443.2 | 443.2 | 7.06 |
| 812 | R.DLSHILGIPQSWR.L | | 507.944 | 3 | 1521.817 | 1521.817 | 0 | −0.3 | 661 | 498.2 | 498.2 | 498.2 | 8.78 |
| 812 | R.DLSHILGIPQSWR.L | | 507.944 | 3 | 1521.817 | 1521.817 | 0 | 0.0 | 661 | 341.8 | 341.8 | 341.8 | 6.04 |
| 2002 | K.DAWRQPEDTWAALEGLSFSPFR.E | | 860.415 | 3 | 2579.231 | 2579.231 | 0 | −0.1 | 710 | 421.6 | 421.6 | 421.6 | 7.21 |
| 2002 | K.DAWRQPEDTWAALEGLSFSPFR.E | | 860.415 | 3 | 2579.231 | 2579.231 | 0 | −0.2 | 710 | 374.7 | 374.7 | 374.7 | 7.09 |
| 1133 | R.QPEDTWAALEGLSFSPFREQMLDTSSLLQFMR.E | | 933.452 | 4 | 3730.787 | 3730.788 | 0 | −0.3 | 714 | 659.7 | 659.7 | 659.7 | 10.32 |
| 885 | R.QPEDTWAALEGLSFSPFR.E | | 1025.998 | 2 | 2050.988 | 2050.987 | 0 | 0.4 | 714 | 533.0 | 533.0 | 533.0 | 9.12 |
| 1133 | R.QPEDTWAALEGLSFSPFREQMLDTSSLLQFMR.E | | 1244.267 | 3 | 3730.786 | 3730.788 | 0 | −0.5 | 714 | 601.9 | 601.9 | 601.9 | 8.94 |
| 1133 | R.QPEDTWAALEGLSFSPFREQMLDTSSLLQFMR.E | | 933.453 | 4 | 3730.789 | 3730.788 | 0 | 0.2 | 714 | 595.7 | 595.7 | 595.7 | 7.77 |
| 885 | R.QPEDTWAALEGLSFSPFR.E | | 684.334 | 3 | 2050.987 | 2050.987 | 0 | 0.1 | 714 | 429.9 | 429.9 | 429.9 | 7.53 |
| 1616 | R.QPEDTWAALEGLSFSPFREQM[+15.995]LDTSSLLQFMR.E | M[+16] | 1249.936 | 3 | 3747.792 | 3746.783 | 1 | 1.6 | 714 | 512.3 | 512.3 | 144.5 | 7.50 |
| 949 | R.EQMLDTSSLLQFMR.E | | 849.914 | 2 | 1698.820 | 1698.819 | 0 | 0.6 | 732 | 667.1 | 667.1 | 667.1 | 11.58 |
| 949 | R.EQMLDTSSLLQFMR.E | | 849.914 | 2 | 1698.820 | 1698.819 | 0 | 0.6 | 732 | 586.3 | 586.3 | 586.3 | 9.58 |
| 949 | R.EQMLDTSSLLQFMR.E | | 566.945 | 3 | 1698.820 | 1698.819 | 0 | 0.5 | 732 | 493.2 | 493.2 | 493.2 | 7.69 |
| 1558 | R.EQM[+15.995]LDTSSLLQFMR.E | M[+16] | 572.277 | 3 | 1714.815 | 1714.814 | 0 | 0.6 | 732 | 411.8 | 411.8 | 411.8 | 7.10 |
| 1860 | R.EKQHLLSIDEPLFR.S | | 431.989 | 4 | 1724.933 | 1724.933 | 0 | 0.1 | 746 | 350.9 | 350.9 | 350.9 | 5.98 |
| 1860 | R.EKQHLLSIDEPLFR.S | | 575.649 | 3 | 1724.933 | 1724.933 | 0 | 0.2 | 746 | 301.6 | 301.6 | 301.6 | 5.50 |
| 2190 | K.QHLLSIDEPLFR.S | | 489.937 | 3 | 1467.795 | 1467.795 | 0 | −0.1 | 748 | 429.5 | 429.5 | 429.5 | 7.86 |
| 3084 | R.LLSLVDSAGQRDETGN[+0.984]NSVQTVFQGTLAATKR.W | N[+1] | 845.180 | 4 | 3377.696 | 3377.730 | 0 | −9.9 | 881 | 319.2 | 319.2 | 26.7 | 2.36 |
| 1226 | R.DETGNNSVQTVFQGTLAATKR.W | | 746.377 | 3 | 2237.117 | 2237.116 | 0 | 0.4 | 892 | 661.4 | 661.4 | 661.4 | 11.53 |
| 867 | R.LVEIQFPAEHGWKESLLGDMEWR.L | | 924.796 | 3 | 2772.373 | 2770.366 | 2 | 0.1 | 942 | 509.9 | 509.9 | 509.9 | 7.95 |
| 867 | R.LVEIQFPAEHGWKESLLGDMEWR.L | | 693.347 | 4 | 2770.365 | 2770.366 | 0 | −0.3 | 942 | 448.5 | 448.5 | 448.5 | 7.75 |
| 867 | R.LVEIQFPAEHGWKESLLGDMEWR.L | | 693.347 | 4 | 2770.368 | 2770.366 | 0 | 0.8 | 942 | 391.7 | 389.2 | 389.2 | 7.47 |
| 867 | R.LVEIQFPAEHGWKESLLGDMEWR.L | | 924.466 | 3 | 2771.382 | 2770.366 | 1 | 4.6 | 942 | 394.3 | 394.3 | 394.3 | 5.67 |
| 4202 | K.ESLLGDMEWR.L | | 618.290 | 2 | 1235.572 | 1235.573 | 0 | −0.2 | 955 | 558.0 | 558.0 | 558.0 | 8.91 |
| 996 | K.GLEDSVAKTFEK.C | | 662.344 | 2 | 1323.680 | 1323.679 | 0 | 0.6 | 986 | 594.6 | 594.6 | 594.6 | 8.77 |
| 996 | K.GLEDSVAKTFEK.C | | 441.898 | 3 | 1323.679 | 1323.679 | 0 | 0.0 | 986 | 526.7 | 526.7 | 526.7 | 8.65 |
| 996 | K.GLEDSVAKTFEK.C | | 441.898 | 3 | 1323.679 | 1323.679 | 0 | −0.2 | 986 | 337.1 | 337.1 | 337.1 | 5.32 |
| 972 | K.FGIVLSAVITK.S | | 574.358 | 2 | 1147.708 | 1147.709 | 0 | −0.1 | 1025 | 483.9 | 483.9 | 483.9 | 8.01 |
| 972 | K.FGIVLSAVITK.S | | 574.358 | 2 | 1147.709 | 1147.709 | 0 | 0.0 | 1025 | 401.9 | 401.9 | 401.9 | 7.64 |
| 913 | R.TADNFDDILKHLLTLADVK.H | | 714.720 | 3 | 2142.147 | 2142.144 | 0 | 1.1 | 1040 | 726.9 | 726.9 | 726.9 | 12.53 |
| 913 | R.TADNFDDILKHLLTLADVK.H | | 536.292 | 4 | 2142.144 | 2142.144 | 0 | 0.0 | 1040 | 571.5 | 571.5 | 571.5 | 9.53 |
| 913 | R.TADNFDDILKHLLTLADVK.H | | 536.542 | 4 | 2143.146 | 2142.144 | 1 | −0.8 | 1040 | 508.7 | 508.7 | 178.6 | 7.81 |
| 817 | R.TADNFDDILK.H | | 576.283 | 2 | 1151.558 | 1151.558 | 0 | 0.2 | 1040 | 368.0 | 368.0 | 368.0 | 7.14 |
| 1060 | K.HLLTLADVK.H | | 505.306 | 2 | 1009.604 | 1009.604 | 0 | −0.1 | 1050 | 549.5 | 549.5 | 549.5 | 8.14 |
| 1724 | R.LC[+57.021]GTDEKILANVTEDAKR.L | C[+57] | 509.014 | 4 | 2033.033 | 2033.033 | 0 | 0.1 | 1063 | 566.5 | 566.5 | 566.5 | 9.19 |
| 1724 | R.LC[+57.021]GTDEKILANVTEDAKR.L | C[+57] | 678.350 | 3 | 2033.034 | 2033.033 | 0 | 0.4 | 1063 | 507.6 | 507.6 | 507.6 | 8.39 |
| 951 | K.ILANVTEDAKR.L | | 615.346 | 2 | 1229.684 | 1229.685 | 0 | −0.4 | 1070 | 565.2 | 565.2 | 565.2 | 9.32 |
| 951 | K.ILANVTEDAKR.L | | 410.566 | 3 | 1229.685 | 1229.685 | 0 | −0.1 | 1070 | 559.3 | 559.3 | 559.3 | 8.65 |
| 951 | K.ILANVTEDAKR.L | | 410.566 | 3 | 1229.685 | 1229.685 | 0 | −0.1 | 1070 | 546.6 | 546.6 | 546.6 | 8.65 |
| 951 | K.ILANVTEDAKR.L | | 410.566 | 3 | 1229.685 | 1229.685 | 0 | −0.2 | 1070 | 444.6 | 444.6 | 444.6 | 7.54 |
| 951 | K.ILANVTEDAKR.L | | 410.567 | 3 | 1229.685 | 1229.685 | 0 | 0.0 | 1070 | 429.2 | 429.2 | 429.2 | 7.38 |
| 829 | K.ILANVTEDAK.R | | 537.295 | 2 | 1073.584 | 1073.584 | 0 | −0.2 | 1070 | 500.6 | 333.1 | 333.1 | 7.05 |
| 1772 | K.RLIAVADSVLTK.V | | 429.266 | 3 | 1285.784 | 1285.784 | 0 | −0.2 | 1080 | 444.8 | 444.8 | 444.8 | 7.80 |
| 1772 | K.RLIAVADSVLTK.V | | 643.396 | 2 | 1285.784 | 1285.784 | 0 | −0.1 | 1080 | 325.1 | 325.1 | 325.1 | 5.58 |
| 2210 | R.LIAVADSVLTK.V | | 565.345 | 2 | 1129.682 | 1129.683 | 0 | −0.2 | 1081 | 523.4 | 523.4 | 523.4 | 8.14 |
| 2210 | R.LIAVADSVLTK.V | | 565.345 | 2 | 1129.683 | 1129.683 | 0 | −0.1 | 1081 | 416.3 | 270.4 | 270.4 | 6.17 |
| 1220 | K.VVGDLLSGTILVGQLELIIK.H | | 694.093 | 3 | 2080.265 | 2080.263 | 0 | 1.0 | 1092 | 580.5 | 580.5 | 580.5 | 9.25 |
| 1973 | K.HKNQFLDIWQLR.E | | 533.291 | 3 | 1597.859 | 1597.860 | 0 | −0.3 | 1112 | 377.1 | 377.1 | 377.1 | 7.06 |
| 3513 | K.HKNQFLDIWQLREK.S | | 464.505 | 4 | 1854.998 | 1854.997 | 0 | 0.4 | 1112 | 375.6 | 375.6 | 375.6 | 6.73 |
| 3513 | K.HKNQFLDIWQLREK.S | | 464.505 | 4 | 1854.999 | 1854.997 | 0 | 0.8 | 1112 | 337.0 | 337.0 | 337.0 | 5.26 |
| 1973 | K.HKNQFLDIWQLR.E | | 533.626 | 3 | 1598.863 | 1597.860 | 1 | −0.4 | 1112 | 307.1 | 307.1 | 307.1 | 3.54 |
| 2135 | K.NQFLDIWQLR.E | | 666.857 | 2 | 1332.707 | 1332.706 | 0 | 0.5 | 1114 | 498.0 | 433.0 | 433.0 | 8.01 |
| 2135 | K.NQFLDIWQLR.E | | 444.907 | 3 | 1332.706 | 1332.706 | 0 | 0.0 | 1114 | 465.8 | 465.8 | 465.8 | 7.06 |
| 2369 | K.NQFLDIWQLREK.S | | 530.619 | 3 | 1589.843 | 1589.844 | 0 | −0.6 | 1114 | 439.5 | 439.5 | 439.5 | 6.46 |
| 1763 | R.REELLLLK.K | | 507.321 | 2 | 1013.635 | 1013.635 | 0 | −0.3 | 1144 | 345.3 | 345.3 | 345.3 | 5.58 |
| 3455 | R.REELLLLKK.E | | 571.369 | 2 | 1141.730 | 1141.730 | 0 | 0.0 | 1144 | 314.3 | 314.3 | 314.3 | 3.58 |
| 989 | R.EELLLLKK.E | | 493.318 | 2 | 985.629 | 985.629 | 0 | −0.1 | 1145 | 518.7 | 518.7 | 518.7 | 7.97 |
| 2221 | R.EELLLLK.K | | 429.271 | 2 | 857.534 | 857.534 | 0 | 0.2 | 1145 | 452.9 | 452.9 | 452.9 | 7.80 |
| 989 | R.EELLLLKK.E | | 493.318 | 2 | 985.629 | 985.629 | 0 | 0.0 | 1145 | 400.1 | 400.1 | 400.1 | 7.00 |
| 989 | R.EELLLLKK.E | | 493.318 | 2 | 985.629 | 985.629 | 0 | 0.1 | 1145 | 356.1 | 356.1 | 356.1 | 5.60 |
| 1703 | R.C[+57.021]VDSLLK.M | C[+57] | 417.723 | 2 | 834.439 | 834.439 | 0 | 0.1 | 1156 | 466.3 | 466.3 | 466.3 | 7.42 |
| 1703 | R.C[+57.021]VDSLLK.M | C[+57] | 417.723 | 2 | 834.439 | 834.439 | 0 | −0.2 | 1156 | 436.2 | 436.2 | 436.2 | 7.26 |
| 926 | K.HLIQVDFGVLAVR.H | | 489.621 | 3 | 1466.848 | 1466.848 | 0 | 0.4 | 1169 | 456.6 | 456.6 | 456.6 | 8.02 |
| 926 | K.HLIQVDFGVLAVR.H | | 489.621 | 3 | 1466.848 | 1466.848 | 0 | 0.1 | 1169 | 446.4 | 446.4 | 446.4 | 7.80 |
| 926 | K.HLIQVDFGVLAVR.H | | 489.621 | 3 | 1466.848 | 1466.848 | 0 | −0.1 | 1169 | 395.2 | 395.2 | 395.2 | 7.53 |
| 926 | K.HLIQVDFGVLAVR.H | | 489.621 | 3 | 1466.847 | 1466.848 | 0 | −0.6 | 1169 | 501.7 | 501.7 | 501.7 | 7.31 |
| 1160 | R.HSQDLSSKRLNDTVTVR.L | | 489.762 | 4 | 1956.026 | 1956.026 | 0 | 0.1 | 1182 | 558.2 | 558.2 | 558.2 | 8.52 |
| 2114 | R.LNDTVTVR.L | | 459.256 | 2 | 917.505 | 917.505 | 0 | 0.0 | 1191 | 366.6 | 366.6 | 366.6 | 6.87 |
| 811 | R.ATHYHLSSQVQEMAGKIDLLR.D | | 600.064 | 4 | 2397.236 | 2397.234 | 0 | 0.6 | 1208 | 698.1 | 698.1 | 698.1 | 11.53 |
| 1562 | R.ATHYHLSSQVQEM[+15.995]AGK.I | M[+16] | 601.621 | 3 | 1802.849 | 1802.849 | 0 | −0.2 | 1208 | 604.1 | 604.1 | 604.1 | 10.58 |
| 1562 | R.ATHYHLSSQVQEM[+15.995]AGK.I | M[+16] | 601.622 | 3 | 1802.850 | 1802.849 | 0 | 0.6 | 1208 | 600.4 | 600.4 | 600.4 | 10.58 |
| 1012 | R.ATHYHLSSQVQEMAGK.I | | 893.931 | 2 | 1786.854 | 1786.854 | 0 | 0.2 | 1208 | 662.9 | 662.9 | 662.9 | 10.24 |
| 1562 | R.ATHYHLSSQVQEM[+15.995]AGK.I | M[+16] | 601.621 | 3 | 1802.850 | 1802.849 | 0 | 0.4 | 1208 | 531.3 | 531.3 | 531.3 | 8.91 |
| 1012 | R.ATHYHLSSQVQEMAGK.I | | 596.290 | 3 | 1786.855 | 1786.854 | 0 | 0.5 | 1208 | 509.4 | 509.4 | 509.4 | 8.78 |
| 1562 | R.ATHYHLSSQVQEM[+15.995]AGK.I | M[+16] | 451.468 | 4 | 1802.850 | 1802.849 | 0 | 0.3 | 1208 | 460.3 | 460.3 | 460.3 | 8.57 |
| 1012 | R.ATHYHLSSQVQEMAGK.I | | 447.469 | 4 | 1786.856 | 1786.854 | 0 | 1.0 | 1208 | 443.5 | 443.5 | 443.5 | 8.57 |
| 1012 | R.ATHYHLSSQVQEMAGK.I | | 596.289 | 3 | 1786.854 | 1786.854 | 0 | −0.2 | 1208 | 524.1 | 524.1 | 524.1 | 8.36 |
| 1012 | R.ATHYHLSSQVQEMAGK.I | | 447.469 | 4 | 1786.855 | 1786.854 | 0 | 0.2 | 1208 | 512.4 | 512.4 | 512.4 | 8.23 |
| 1562 | R.ATHYHLSSQVQEM[+15.995]AGK.I | M[+16] | 451.468 | 4 | 1802.849 | 1802.849 | 0 | −0.3 | 1208 | 419.5 | 405.3 | 405.3 | 7.86 |
| 1012 | R.ATHYHLSSQVQEMAGK.I | | 447.470 | 4 | 1786.856 | 1786.854 | 0 | 1.2 | 1208 | 398.1 | 398.1 | 398.1 | 7.75 |
| 1562 | R.ATHYHLSSQVQEM[+15.995]AGK.I | M[+16] | 601.622 | 3 | 1802.851 | 1802.849 | 0 | 0.9 | 1208 | 415.4 | 415.4 | 415.4 | 7.64 |
| 1012 | R.ATHYHLSSQVQEMAGK.I | | 447.469 | 4 | 1786.856 | 1786.854 | 0 | 1.0 | 1208 | 406.4 | 406.4 | 406.4 | 7.64 |
| 1562 | R.ATHYHLSSQVQEM[+15.995]AGK.I | M[+16] | 451.468 | 4 | 1802.848 | 1802.849 | 0 | −0.5 | 1208 | 367.6 | 367.6 | 367.6 | 6.41 |
| 1012 | R.ATHYHLSSQVQEMAGK.I | | 447.469 | 4 | 1786.855 | 1786.854 | 0 | 0.2 | 1208 | 307.7 | 307.7 | 307.7 | 5.97 |
| 1012 | R.ATHYHLSSQVQEMAGK.I | | 447.469 | 4 | 1786.855 | 1786.854 | 0 | 0.6 | 1208 | 315.1 | 315.1 | 315.1 | 5.76 |
| 1012 | R.ATHYHLSSQVQEMAGK.I | | 447.469 | 4 | 1786.854 | 1786.854 | 0 | −0.2 | 1208 | 315.1 | 315.1 | 315.1 | 5.58 |
| 3483 | K.IDLLRDSHIFQLFWR.E | | 490.521 | 4 | 1959.061 | 1959.060 | 0 | 0.4 | 1224 | 314.5 | 314.5 | 314.5 | 5.32 |
| 838 | R.DSHIFQLFWR.E | | 674.843 | 2 | 1348.680 | 1348.680 | 0 | −0.1 | 1229 | 554.0 | 554.0 | 554.0 | 8.36 |
| 838 | R.DSHIFQLFWR.E | | 450.231 | 3 | 1348.679 | 1348.680 | 0 | −0.3 | 1229 | 443.3 | 443.3 | 443.3 | 7.60 |
| 1380 | R.EAAEPLSEPKEDQEAAELLSEPEEESERHILELEEVYDYLYQPS | | 1363.889 | 4 | 5452.536 | 5452.539 | 0 | −0.6 | 1239 | 936.9 | 739.0 | 739.0 | 14.21 |
| | YR.K | | | | | | | | | | | | |
| 1469 | R.EAAEPLSEPKEDQEAAELLSEPEEES[+79.966]ERHILELEE | S[+80] | 1383.883 | | 5532.509 | 5532.505 | 0 | 0.5 | 1239 | 796.8 | 779.7 | 146.9 | 12.13 |
| | VYDYLYQPSYR.K | | | | | | | | | | | | |
| 1380 | R.EAAEPLSEPKEDQEAAELLSEPEEESERHILELEEVYDYLYQPS | | 1363.889 | 4 | 5452.536 | 5452.539 | 0 | −0.6 | 1239 | 683.5 | 683.5 | 683.5 | 9.22 |
| | YR.K | | | | | | | | | | | | |
| 1380 | R.EAAEPLSEPKEDQEAAELLSEPEEESERHILELEEVYDYLYQPS | | 1091.313 | | 5452.537 | 5452.539 | 0 | −0.4 | 1239 | 627.1 | 627.1 | 627.1 | 9.13 |
| | YR.K | | | | | | | | | | | | |
| 1468 | R.EAAEPLSEPKEDQEAAELLS[+79.966]EPEEESERHILELEE | S[+80] | 1107.308 | 5 | 5532.511 | 5532.505 | 0 | 1.1 | 1239 | 610.7 | 610.7 | 3.5 | 9.13 |
| | VYDYLYQPSYR.K | | | | | | | | | | | | |
| 1299 | R.EAAEPLSEPKEDQEAAELLSEPEEESER.H | | 1047.812 | 3 | 3141.421 | 3141.423 | 0 | −0.6 | 1239 | 599.6 | 599.6 | 599.6 | 8.61 |
| 3076 | R.EAAEPLSEPKEDQEAAELLS[+79.966]EPEEESER.H | S[+80] | 1074.802 | 3 | 3222.391 | 3221.389 | 1 | −0.6 | 1239 | 582.1 | 582.1 | 96.3 | 8.61 |
| 1380 | R.EAAEPLSEPKEDQEAAELLSEPEEESERHILELEEVYDYLYQPS | | 1091.314 | 5 | 5452.539 | 5452.539 | 0 | 0.0 | 1239 | 541.8 | 541.8 | 541.8 | 6.91 |
| | YR.K | | | | | | | | | | | | |
| 1299 | R.EAAEPLSEPKEDQEAAELLSEPEEESER.H | | 786.362 | 4 | 3142.425 | 3141.423 | 1 | −0.3 | 1239 | 449.8 | 449.8 | 449.8 | 6.55 |
| 3065 | R.EAAEPLSEPKEDQEAAELLSEPEEESERHILELEEVY | Y[+80] | 922.924 | 6 | 5532.510 | 5532.505 | 0 | 0.8 | 1239 | 496.0 | 480.6 | 27.7 | 5.82 |
| | [+79.966]DYLYQPSYR.K | | | | | | | | | | | | |
| 1380 | R.EAAEPLSEPKEDQEAAELLSEPEEESERHILELEEVYDYLYQPS | | 909.763 | 6 | 5453.540 | 5452.539 | 1 | −0.4 | 1239 | 446.8 | 446.8 | 446.8 | 5.61 |
| | YR.K | | | | | | | | | | | | |
| 3065 | R.EAAEPLSEPKEDQEAAELLSEPEEESERHILELEEVY | Y[+80] | 1107.708 | 5 | 5534.508 | 5532.505 | 2 | −0.7 | 1239 | 443.9 | 443.9 | 7.1 | 4.78 |
| | [+79.966]DYLYQPSYR.K | | | | | | | | | | | | |
| 3475 | R.HILELEEVYDYLYQPSYR.K | | 777.382 | 3 | 2330.133 | 2330.134 | 0 | −0.6 | 1267 | 302.8 | 302.8 | 302.8 | 4.87 |
| 2956 | K.SGEVTLAEIDVIFKDFVNK.Y | | 708.713 | 3 | 2124.123 | 2124.122 | 0 | 0.4 | 1295 | 543.4 | 543.4 | 543.4 | 8.86 |
| 3848 | K.SGEVTLAEIDVIFKDFVNKYTDLDSELK.I | | 797.913 | 4 | 3188.629 | 3188.625 | 0 | 1.2 | 1295 | 413.7 | 413.7 | 413.7 | 7.05 |
| 3923 | K.YTDLDSELK.I | | 542.264 | 2 | 1083.521 | 1083.520 | 0 | 0.3 | 1314 | 459.9 | 402.4 | 402.4 | 7.60 |
| 2161 | K.DRVEQIKEYHHLHQAVHAAK.V | | 482.657 | 5 | 2409.254 | 2409.253 | 0 | 0.1 | 1338 | 496.5 | 496.5 | 496.5 | 7.04 |
| 2161 | K.DRVEQIKEYHHLHQAVHAAK.V | | 402.382 | 6 | 2409.255 | 2409.253 | 0 | 0.8 | 1338 | 495.3 | 495.3 | 495.3 | 7.04 |
| 2161 | K.DRVEQIKEYHHLHQAVHAAK.V | | 402.382 | 6 | 2409.256 | 2409.253 | 0 | 0.8 | 1338 | 323.5 | 323.5 | 323.5 | 4.31 |
| 2161 | K.DRVEQIKEYHHLHQAVHAAK.V | | 482.657 | 5 | 2409.255 | 2409.253 | 0 | 0.6 | 1338 | 313.6 | 313.6 | 313.6 | 4.03 |
| 2161 | K.DRVEQIKEYHHLHQAVHAAK.V | | 402.382 | 6 | 2409.255 | 2409.253 | 0 | 0.5 | 1338 | 304.4 | 226.0 | 226.0 | 3.95 |
| 1892 | R.VEQIKEYHHLHQAVHAAK.V | | 428.431 | 5 | 2138.126 | 2138.125 | 0 | 0.3 | 1340 | 359.7 | 359.7 | 359.7 | 4.64 |
| 1892 | R.VEQIKEYHHLHQAVHAAK.V | | 428.431 | 5 | 2138.128 | 2138.125 | 0 | 1.0 | 1340 | 311.0 | 311.0 | 311.0 | 4.37 |
| 1892 | R.VEQIKEYHHLHQAVHAAK.V | | 428.432 | 5 | 2138.130 | 2138.125 | 0 | 2.0 | 1340 | 301.8 | 301.8 | 301.8 | 3.07 |
| 946 | R.ETLDQINQELIQAK.K | | 821.936 | 2 | 1642.864 | 1642.865 | 0 | −0.3 | 1391 | 619.7 | 619.7 | 619.7 | 10.58 |
| 1077 | R.ETLDQINQELIQAKK.L | | 590.992 | 3 | 1770.960 | 1770.960 | 0 | 0.1 | 1391 | 614.8 | 614.8 | 614.8 | 10.32 |
| 946 | R.ETLDQINQELIQAK.K | | 821.936 | 2 | 1642.865 | 1642.865 | 0 | 0.5 | 1391 | 558.0 | 550.7 | 550.7 | 8.91 |
| 946 | R.ETLDQINQELIQAK.K | | 548.293 | 3 | 1642.866 | 1642.865 | 0 | 0.5 | 1391 | 380.2 | 380.2 | 380.2 | 6.79 |
| 1101 | K.KLLQDISEAR.C | | 586.835 | 2 | 1172.663 | 1172.663 | 0 | 0.0 | 1405 | 604.8 | 514.5 | 514.5 | 9.81 |
| 998 | K.LLQDISEAR.C | | 522.788 | 2 | 1044.568 | 1044.568 | 0 | −0.2 | 1406 | 519.8 | 303.5 | 303.5 | 6.65 |
| 998 | K.LLQDISEAR.C | | 522.788 | 2 | 1044.568 | 1044.568 | 0 | 0.0 | 1406 | 479.9 | 285.6 | 285.6 | 6.44 |
| 4530 | R.EALGGINELKVFVDLASISAGENDIDVDR.V | | 1020.524 | 3 | 3059.556 | 3059.553 | 0 | 0.9 | 1433 | 714.3 | 714.3 | 714.3 | 11.85 |
| 2286 | R.EALGGINELK.V | | 522.290 | 2 | 1043.573 | 1043.573 | 0 | −0.2 | 1433 | 446.4 | 446.4 | 446.4 | 7.80 |
| 2286 | R.EALGGINELK.V | | 522.290 | 2 | 1043.573 | 1043.573 | 0 | −0.6 | 1433 | 455.3 | 455.3 | 455.3 | 7.11 |
| 2191 | K.ALDKDQYLPR.K | | 609.828 | 2 | 1218.648 | 1218.648 | 0 | 0.1 | 1498 | 505.5 | 505.5 | 505.5 | 7.54 |
| 2191 | K.ALDKDQYLPR.K | | 609.828 | 2 | 1218.649 | 1218.648 | 0 | 0.6 | 1498 | 477.5 | 477.5 | 477.5 | 7.54 |
| 2191 | K.ALDKDQYLPR.K | | 406.887 | 3 | 1218.648 | 1218.648 | 0 | −0.1 | 1498 | 467.0 | 467.0 | 467.0 | 7.54 |
| 2191 | K.ALDKDQYLPR.K | | 406.887 | 3 | 1218.648 | 1218.648 | 0 | 0.0 | 1498 | 422.2 | 422.2 | 422.2 | 7.38 |
| 1029 | K.ALDKDQYLPRK.L | | 449.586 | 3 | 1346.743 | 1346.743 | 0 | 0.1 | 1498 | 459.0 | 459.0 | 459.0 | 7.21 |
| 1029 | K.ALDKDQYLPRK.L | | 449.586 | 3 | 1346.743 | 1346.743 | 0 | 0.3 | 1498 | 370.9 | 370.9 | 370.9 | 6.93 |
| 1029 | K.ALDKDQYLPRK.L | | 449.586 | 3 | 1346.743 | 1346.743 | 0 | −0.1 | 1498 | 415.9 | 415.9 | 415.9 | 6.84 |
| 2191 | K.ALDKDQYLPR.K | | 406.888 | 3 | 1218.648 | 1218.648 | 0 | 0.1 | 1498 | 348.7 | 348.7 | 348.7 | 5.60 |
| 832 | R.NLEWLKTVNESHGSVER.S | | 500.257 | 4 | 1998.005 | 1998.004 | 0 | 0.3 | 1515 | 389.5 | 389.5 | 389.5 | 7.47 |
| 832 | R.NLEWLKTVNESHGSVER.S | | 500.256 | 4 | 1998.003 | 1998.004 | 0 | −0.4 | 1515 | 443.4 | 443.4 | 443.4 | 6.83 |
| 832 | R.NLEWLKTVNESHGSVER.S | | 500.256 | 4 | 1998.003 | 1998.004 | 0 | −0.3 | 1515 | 330.2 | 330.2 | 330.2 | 5.99 |
| 2033 | K.TVNESHGSVER.S | | 405.530 | 3 | 1214.577 | 1214.576 | 0 | 0.5 | 1521 | 443.9 | 443.9 | 443.9 | 8.02 |
| 3644 | K.TVNESHGSVERSSLTLATAINQR.G | | 618.321 | 4 | 2470.263 | 2470.265 | 0 | −0.4 | 1521 | 429.9 | 429.9 | 429.9 | 7.59 |
| 3644 | K.TVNESHGSVERSSLTLATAINQR.G | | 618.321 | 4 | 2470.263 | 2470.265 | 0 | −0.4 | 1521 | 308.8 | 308.8 | 308.8 | 5.71 |
| 808 | R.SSLTLATAINQR.G | | 637.857 | 2 | 1274.707 | 1274.706 | 0 | 0.6 | 1532 | 696.8 | 696.8 | 696.8 | 11.58 |
| 808 | R.SSLTLATAINQR.G | | 637.857 | 2 | 1274.707 | 1274.706 | 0 | 0.3 | 1532 | 631.3 | 631.3 | 631.3 | 10.03 |
| 808 | R.SSLTLATAINQR.G | | 637.857 | 2 | 1274.706 | 1274.706 | 0 | −0.1 | 1532 | 524.0 | 524.0 | 524.0 | 8.91 |
| 808 | R.SSLTLATAINQR.G | | 637.857 | 2 | 1274.706 | 1274.706 | 0 | 0.0 | 1532 | 557.1 | 557.1 | 557.1 | 8.14 |
| 808 | R.SSLTLATAINQR.G | | 637.857 | 2 | 1274.707 | 1274.706 | 0 | 0.2 | 1532 | 520.1 | 369.2 | 369.2 | 8.14 |
| 808 | R.SSLTLATAINQR.G | | 637.857 | 2 | 1274.707 | 1274.706 | 0 | 0.2 | 1532 | 472.8 | 472.8 | 472.8 | 7.80 |
| 808 | R.SSLTLATAINQR.G | | 637.857 | 2 | 1274.707 | 1274.706 | 0 | 0.3 | 1532 | 455.7 | 455.7 | 455.7 | 7.80 |
| 808 | R.SSLTLATAINQR.G | | 425.574 | 3 | 1274.706 | 1274.706 | 0 | 0.1 | 1532 | 390.4 | 390.4 | 390.4 | 7.21 |
| 808 | R.SSLTLATAINQR.G | | 637.857 | 2 | 1274.707 | 1274.706 | 0 | 0.5 | 1532 | 353.0 | 353.0 | 353.0 | 6.04 |
| 808 | R.SSLTLATAINQR.G | | 425.908 | 3 | 1275.709 | 1274.706 | 1 | −0.2 | 1532 | 366.8 | 366.8 | 366.8 | 5.20 |
| 847 | R.GIYVIQAPKGGQK.I | | 453.598 | 3 | 1358.779 | 1358.779 | 0 | 0.2 | 1544 | 563.6 | 563.6 | 563.6 | 9.32 |
| 847 | R.GIYVIQAPKGGQK.I | | 679.893 | 2 | 1358.780 | 1358.779 | 0 | 0.3 | 1544 | 515.2 | 515.2 | 515.2 | 8.52 |
| 847 | R.GIYVIQAPKGGQK.I | | 453.598 | 3 | 1358.779 | 1358.779 | 0 | 0.1 | 1544 | 517.4 | 517.4 | 517.4 | 7.97 |
| 813 | R.GIYVIQAPK.G | | 494.795 | 2 | 988.582 | 988.583 | 0 | −0.3 | 1544 | 444.5 | 444.5 | 444.5 | 7.60 |
| 813 | R.GIYVIQAPK.G | | 494.795 | 2 | 988.582 | 988.583 | 0 | −0.3 | 1544 | 439.1 | 439.1 | 439.1 | 7.44 |
| 847 | R.GIYVIQAPKGGQK.I | | 453.598 | 3 | 1358.779 | 1358.779 | 0 | −0.1 | 1544 | 428.6 | 428.6 | 428.6 | 7.18 |
| 1331 | K.ISPDTVLHLILPESPGSHEESR.E | | 604.064 | 4 | 2413.235 | 2413.236 | 0 | −0.3 | 1557 | 581.7 | 562.5 | 562.5 | 9.79 |
| 937 | K.ISPDTVLHLILPESPGSHEESREYSLEEVKELLNK.L | | 798.418 | 5 | 3988.059 | 3988.055 | 0 | 0.9 | 1557 | 392.6 | 392.6 | 392.6 | 6.68 |
| 937 | K.ISPDTVLHLILPESPGSHEESREYSLEEVKELLNK.L | | 1330.025 | 3 | 3988.061 | 3988.055 | 0 | 1.5 | 1557 | 565.9 | 565.9 | 565.9 | 6.62 |
| 937 | K.ISPDTVLHLILPESPGSHEESREYSLEEVKELLNK.L | | 570.586 | 7 | 3988.057 | 3988.055 | 0 | 0.4 | 1557 | 396.5 | 396.5 | 396.5 | 5.72 |
| 937 | K.ISPDTVLHLILPESPGSHEESREYSLEEVKELLNK.L | | 997.770 | 4 | 3988.057 | 3988.055 | 0 | 0.4 | 1557 | 508.5 | 508.5 | 508.5 | 5.59 |
| 937 | K.ISPDTVLHLILPESPGSHEESREYSLEEVKELLNK.L | | 665.515 | 6 | 3988.056 | 3988.055 | 0 | 0.3 | 1557 | 334.0 | 334.0 | 334.0 | 5.17 |
| 937 | K.ISPDTVLHLILPESPGSHEESREYSLEEVKELLNK.L | | 997.769 | 4 | 3988.055 | 3988.055 | 0 | 0.0 | 1557 | 438.9 | 438.9 | 438.9 | 5.03 |
| 937 | K.ISPDTVLHLILPESPGSHEESREYSLEEVKELLNK.L | | 570.586 | 7 | 3988.057 | 3988.055 | 0 | 0.5 | 1557 | 346.6 | 346.6 | 346.6 | 4.63 |
| 937 | K.ISPDTVLHLILPESPGSHEESREYSLEEVKELLNK.L | | 998.018 | 4 | 3989.050 | 3988.055 | 1 | −2.0 | 1557 | 377.4 | 377.4 | 266.1 | 4.00 |
| 982 | R.EYSLEEVKELLNK.L | | 531.950 | 3 | 1593.837 | 1593.837 | 0 | −0.2 | 1579 | 476.1 | 476.1 | 476.1 | 8.31 |
| 3334 | R.FSEVFC[+57.021]SVQR.L | C[+57] | 420.201 | 3 | 1258.589 | 1258.589 | 0 | 0.1 | 1609 | 428.3 | 428.3 | 428.3 | 6.90 |
| 3097 | R.LSQAFIDLHSAGN[+0.984]MLFR.T | N[+1] | 641.333 | 3 | 1921.985 | 1920.964 | 1 | 9.2 | 1619 | 376.3 | 376.3 | 376.3 | 6.16 |
| 3300 | K.EGGDVTELLAALC[+57.021]R.Q | C[+57] | 752.382 | 2 | 1503.757 | 1503.747 | 0 | 6.4 | 1665 | 320.4 | 320.4 | 320.4 | 4.01 |
| 899 | R.KQPPSDAALTMLSFIK.S | | 582.987 | 3 | 1746.946 | 1746.946 | 0 | −0.1 | 1718 | 363.8 | 363.8 | 363.8 | 7.32 |
| 899 | R.KQPPSDAALTMLSFIK.S | | 582.987 | 3 | 1746.945 | 1746.946 | 0 | −0.3 | 1718 | 304.8 | 304.8 | 304.8 | 5.76 |
| 809 | K.QPPSDAALTMLSFIK.S | | 809.930 | 2 | 1618.852 | 1618.851 | 0 | 0.7 | 1719 | 438.5 | 438.5 | 438.5 | 7.86 |
| 809 | K.QPPSDAALTMLSFIK.S | | 540.289 | 3 | 1618.851 | 1618.851 | 0 | 0.0 | 1719 | 431.9 | 431.9 | 431.9 | 7.32 |
| 809 | K.QPPSDAALTMLSFIK.S | | 809.929 | 2 | 1618.851 | 1618.851 | 0 | 0.2 | 1719 | 348.1 | 348.1 | 348.1 | 7.01 |
| 809 | K.QPPSDAALTMLSFIK.S | | 810.430 | 2 | 1619.854 | 1618.851 | 1 | −0.5 | 1719 | 402.5 | 402.5 | 402.5 | 6.72 |
| 809 | K.QPPSDAALTMLSFIK.S | | 540.288 | 3 | 1618.850 | 1618.851 | 0 | −0.4 | 1719 | 415.9 | 415.9 | 415.9 | 6.18 |
| 809 | K.QPPSDAALTMLSFIK.S | | 540.288 | 3 | 1618.850 | 1618.851 | 0 | −0.4 | 1719 | 384.1 | 384.1 | 384.1 | 6.07 |
| 1683 | K.SNC[+57.021]TLRDVLR.A | C[+57] | 411.884 | 3 | 1233.637 | 1233.637 | 0 | 0.1 | 1734 | 320.1 | 320.1 | 320.1 | 5.42 |
| 1312 | R.RVMEELPLMLLSEFSLVDKLR.I | | 630.350 | 4 | 2518.378 | 2518.377 | 0 | 0.4 | 1759 | 622.3 | 622.3 | 622.3 | 10.53 |
| 1633 | R.RVMEELPLM[+15.995]LLSEFSLVDKLR.I | M[+16] | 634.349 | 4 | 2534.375 | 2534.372 | 0 | 1.0 | 1759 | 578.3 | 578.3 | 327.9 | 9.53 |
| 1633 | R.RVMEELPLM[+15.995]LLSEFSLVDKLR.I | M[+16] | 845.463 | 3 | 2534.376 | 2534.372 | 0 | 1.3 | 1759 | 567.8 | 567.8 | 155.0 | 9.53 |
| 1003 | R.RVMEELPLMLLSEFSLVDK.L | | 750.403 | 3 | 2249.193 | 2249.192 | 0 | 0.4 | 1759 | 466.3 | 466.3 | 466.3 | 8.01 |
| 1633 | R.RVMEELPLM[+15.995]LLSEFSLVDKLR.I | M[+16] | 634.349 | 4 | 2534.373 | 2534.372 | 0 | 0.4 | 1759 | 393.9 | 393.9 | 29.8 | 7.27 |
| 1633 | R.RVMEELPLM[+15.995]LLSEFSLVDKLR.I | M[+16] | 845.463 | 3 | 2534.375 | 2534.372 | 0 | 1.1 | 1759 | 306.4 | 306.4 | 176.0 | 5.53 |
| 862 | R.VMEELPLMLLSEFSLVDKLR.I | | 788.097 | 3 | 2362.277 | 2362.276 | 0 | 0.5 | 1760 | 809.2 | 809.2 | 809.2 | 14.54 |
| 1626 | R.VM[+15.995]EELPLMLLSEFSLVDKLR.I | M[+16] | 793.429 | 3 | 2378.272 | 2378.271 | 0 | 0.2 | 1760 | 719.7 | 719.7 | 472.4 | 12.53 |
| 1606 | R.VMEELPLM[+15.995]LLSEFSLVDKLR.I | M[+16] | 595.323 | 4 | 2378.271 | 2378.271 | 0 | 0.1 | 1760 | 704.0 | 704.0 | 365.4 | 11.99 |
| 1626 | R.VM[+15.995]EELPLMLLSEFSLVDKLR.I | M[+16] | 793.429 | 3 | 2378.271 | 2378.271 | 0 | 0.0 | 1760 | 696.2 | 696.2 | 95.5 | 11.53 |
| 1606 | R.VMEELPLM[+15.995]LLSEFSLVDKLR.I | M[+16] | 793.429 | 3 | 2378.273 | 2378.271 | 0 | 0.8 | 1760 | 688.3 | 688.3 | 579.5 | 11.53 |
| 862 | R.VMEELPLMLLSEFSLVDKLR.I | | 788.098 | 3 | 2362.278 | 2362.276 | 0 | 0.8 | 1760 | 686.3 | 686.3 | 686.3 | 11.53 |
| 862 | R.VMEELPLMLLSEFSLVDKLR.I | | 1181.643 | 2 | 2362.278 | 2362.276 | 0 | 0.7 | 1760 | 668.5 | 668.5 | 668.5 | 11.53 |
| 862 | R.VMEELPLMLLSEFSLVDKLR.I | | 788.097 | 3 | 2362.277 | 2362.276 | 0 | 0.3 | 1760 | 665.7 | 665.7 | 665.7 | 11.53 |
| 862 | R.VMEELPLMLLSEFSLVDKLR.I | | 591.325 | 4 | 2362.277 | 2362.276 | 0 | 0.5 | 1760 | 698.6 | 698.6 | 698.6 | 10.99 |
| 862 | R.VMEELPLMLLSEFSLVDKLR.I | | 788.097 | 3 | 2362.278 | 2362.276 | 0 | 0.7 | 1760 | 633.9 | 633.9 | 633.9 | 10.53 |
| 1082 | R.VMEELPLMLLSEFSLVDK.L | | 698.369 | 3 | 2093.092 | 2093.091 | 0 | 0.6 | 1760 | 568.5 | 568.5 | 568.5 | 9.25 |
| 1606 | R.VMEELPLM[+15.995]LLSEFSLVDKLR.I | M[+16] | 1189.641 | 2 | 2378.275 | 2378.271 | 0 | 1.8 | 1760 | 626.0 | 626.0 | 395.8 | 9.23 |
| 1082 | R.VMEELPLMLLSEFSLVDK.L | | 1047.050 | 2 | 2093.092 | 2093.091 | 0 | 0.7 | 1760 | 539.4 | 539.4 | 539.4 | 9.12 |
| 1614 | R.VMEELPLM[+15.995]LLSEFSLVDK.L | M[+16] | 1055.047 | 2 | 2109.087 | 2109.086 | 0 | 0.7 | 1760 | 521.3 | 521.3 | 317.2 | 9.12 |
| 1606 | R.VMEELPLM[+15.995]LLSEFSLVDKLR.I | M[+16] | 793.430 | 3 | 2378.274 | 2378.271 | 0 | 1.2 | 1760 | 561.0 | 561.0 | 315.1 | 8.98 |
| 1626 | R.VM[+15.995]EELPLMLLSEFSLVDKLR.I | M[+16] | 793.429 | 3 | 2378.271 | 2378.271 | 0 | 0.1 | 1760 | 543.4 | 543.4 | 316.7 | 8.86 |
| 1614 | R.VMEELPLM[+15.995]LLSEFSLVDK.L | M[+16] | 703.701 | 3 | 2109.087 | 2109.086 | 0 | 0.6 | 1760 | 517.5 | 517.5 | 351.8 | 8.45 |
| 1529 | R.VM[+15.995]EELPLMLLSEFSLVDK.L | M[+16] | 703.701 | 3 | 2109.088 | 2109.086 | 0 | 1.1 | 1760 | 433.3 | 433.3 | 150.0 | 8.08 |
| 1626 | R.VM[+15.995]EELPLMLLSEFSLVDKLR.I | M[+16] | 793.429 | 3 | 2378.272 | 2378.271 | 0 | 0.4 | 1760 | 475.6 | 475.6 | 87.0 | 7.75 |
| 1626 | R.VM[+15.995]EELPLMLLSEFSLVDKLR.I | M[+16] | 793.429 | 3 | 2378.273 | 2378.271 | 0 | 0.8 | 1760 | 366.7 | 366.7 | 205.6 | 7.09 |
| 834 | R.IIMEQSMR.C | | 504.254 | 2 | 1007.501 | 1007.501 | 0 | −0.2 | 1780 | 504.2 | 504.2 | 504.2 | 8.01 |
| 834 | R.IIMEQSMR.C | | 504.254 | 2 | 1007.501 | 1007.501 | 0 | −0.2 | 1780 | 393.8 | 393.8 | 393.8 | 7.14 |
| 1531 | R.IIM[+15.995]EQSMR.C | M[+16] | 512.252 | 2 | 1023.497 | 1023.496 | 0 | 0.5 | 1780 | 353.6 | 353.6 | 25.9 | 5.58 |
| 1531 | R.IIM[+15.995]EQSMR.C | M[+16] | 512.252 | 2 | 1023.496 | 1023.496 | 0 | −0.1 | 1780 | 323.2 | 323.2 | 34.4 | 5.58 |
| 834 | R.IIMEQSMR.C | | 504.254 | 2 | 1007.501 | 1007.501 | 0 | 0.0 | 1780 | 303.4 | 303.4 | 303.4 | 5.31 |
| 1531 | R.IIM[+15.995]EQSMR.C | M[+16] | 512.252 | 2 | 1023.496 | 1023.496 | 0 | −0.2 | 1780 | 319.0 | 319.0 | 11.0 | 5.18 |
| 834 | R.IIMEQSMR.C | | 504.254 | 2 | 1007.501 | 1007.501 | 0 | −0.6 | 1780 | 314.3 | 314.3 | 314.3 | 4.39 |
| 849 | K.VYSLLFADQLSYEVAR.Q | | 937.489 | 2 | 1873.971 | 1873.969 | 0 | 0.7 | 1891 | 514.6 | 514.6 | 514.6 | 8.78 |
| 849 | K.VYSLLFADQLSYEVAR.Q | | 625.328 | 3 | 1873.971 | 1873.969 | 0 | 0.6 | 1891 | 559.8 | 559.8 | 559.8 | 7.82 |
| 851 | K.VFVTPQAPLEAIQAYLAGHYRVPK.Q | | 667.871 | 4 | 2668.461 | 2668.461 | 0 | 0.1 | 1948 | 812.7 | 812.7 | 812.7 | 14.54 |
| 851 | K.VFVTPQAPLEAIQAYLAGHYRVPK.Q | | 534.498 | 5 | 2668.461 | 2668.461 | 0 | 0.1 | 1948 | 766.5 | 766.5 | 766.5 | 12.99 |
| 1411 | K.VFVTPQAPLEAIQAYLAGHYR.V | | 586.817 | 4 | 2344.245 | 2344.245 | 0 | 0.2 | 1948 | 742.5 | 742.5 | 742.5 | 12.25 |
| 1411 | K.VFVTPQAPLEAIQAYLAGHYR.V | | 782.087 | 3 | 2344.245 | 2344.245 | 0 | 0.2 | 1948 | 677.6 | 677.6 | 677.6 | 11.79 |
| 851 | K.VFVTPQAPLEAIQAYLAGHYRVPK.Q | | 667.871 | 4 | 2668.462 | 2668.461 | 0 | 0.5 | 1948 | 644.9 | 644.9 | 644.9 | 10.53 |
| 959 | K.VFVTPQAPLEAIQAYLAGHYRVPKQTLSAAAVENDR.L | | 789.227 | 5 | 3942.105 | 3942.103 | 0 | 0.7 | 1948 | 510.0 | 510.0 | 510.0 | 6.94 |
| 3778 | R.VPKQTLSAAAVFNDR.L | | 539.630 | 3 | 1616.875 | 1616.876 | 0 | 0.5 | 1969 | 389.4 | 389.4 | 389.4 | 6.15 |
| 814 | K.QTLSAAAVFNDR.L | | 646.834 | 2 | 1292.660 | 1292.659 | 0 | 0.3 | 1972 | 637.3 | 557.2 | 557.2 | 10.58 |
| 814 | K.QTLSAAAVFNDR.L | | 646.833 | 2 | 1292.660 | 1292.659 | 0 | 0.1 | 1972 | 551.7 | 551.7 | 551.7 | 8.91 |
| 814 | K.QTLSAAAVFNDR.L | | 646.834 | 2 | 1292.660 | 1292.659 | 0 | 0.5 | 1972 | 510.1 | 510.1 | 510.1 | 8.78 |
| 814 | K.QTLSAAAVFNDR.L | | 646.833 | 2 | 1292.659 | 1292.659 | 0 | −0.2 | 1972 | 320.6 | 320.6 | 320.6 | 6.04 |
| 3457 | K.MKMQLNVK.N | | 496.275 | 2 | 991.542 | 991.543 | 0 | −0.3 | 2009 | 367.7 | 367.7 | 367.7 | 6.48 |
| 3457 | K.MKMQLNVK.N | | 496.275 | 2 | 991.542 | 991.543 | 0 | −0.3 | 2009 | 333.6 | 333.6 | 333.6 | 5.20 |
| 1244 | R.LIDPQVDESRVLGALLPFLDAQYQK.V | | 943.512 | 3 | 2828.523 | 2828.519 | 0 | 1.1 | 2025 | 616.9 | 616.9 | 616.9 | 10.53 |
| 1244 | R.LIDPQVDESRVLGALLPFLDAQYQK.V | | 707.886 | 4 | 2828.522 | 2828.519 | 0 | 1.1 | 2025 | 375.8 | 375.8 | 375.8 | 7.71 |
| 1009 | R.LIDPQVDESR.V | | 586.301 | 2 | 1171.595 | 1171.595 | 0 | −0.5 | 2025 | 543.6 | 389.6 | 389.6 | 7.45 |
| 1244 | R.LIDPQVDESRVLGALLPFLDAQYQK.V | | 943.513 | 3 | 2828.525 | 2828.519 | 0 | 1.9 | 2025 | 405.5 | 405.5 | 405.5 | 6.09 |
| 871 | R.VLGALLPFLDAQYQK.V | | 838.475 | 2 | 1675.943 | 1675.942 | 0 | 0.5 | 2035 | 496.1 | 496.1 | 496.1 | 8.78 |
| 843 | R.TRVPQFSFLDIFPK.V | | 565.647 | 3 | 1694.927 | 1694.927 | 0 | 0.5 | 2116 | 491.0 | 491.0 | 491.0 | 7.97 |
| 843 | R.TRVPQFSFLDIFPK.V | | 565.647 | 3 | 1694.927 | 1694.927 | 0 | 0.3 | 2116 | 464.6 | 464.6 | 464.6 | 7.76 |
| 843 | R.TRVPQFSFLDIFPK.V | | 565.647 | 3 | 1694.926 | 1694.927 | 0 | −0.1 | 2116 | 452.6 | 452.6 | 452.6 | 7.76 |
| 843 | R.TRVPQFSFLDIFPK.V | | 565.648 | 3 | 1694.928 | 1694.927 | 0 | 1.1 | 2116 | 446.1 | 446.1 | 446.1 | 7.76 |
| 843 | R.TRVPQFSFLDIFPK.V | | 565.647 | 3 | 1694.927 | 1694.927 | 0 | 0.3 | 2116 | 401.6 | 401.6 | 401.6 | 7.38 |
| 843 | R.TRVPQFSFLDIFPK.V | | 565.648 | 3 | 1694.928 | 1694.927 | 0 | 0.8 | 2116 | 379.6 | 379.6 | 379.6 | 7.27 |
| 843 | R.TRVPQFSFLDIFPK.V | | 847.966 | 2 | 1694.926 | 1694.927 | 0 | −0.5 | 2116 | 454.4 | 454.4 | 454.4 | 6.63 |
| 843 | R.TRVPQFSFLDIFPK.V | | 847.966 | 2 | 1694.926 | 1694.927 | 0 | −0.5 | 2116 | 453.6 | 453.6 | 453.6 | 6.63 |
| 843 | R.TRVPQFSFLDIFPK.V | | 847.966 | 2 | 1694.926 | 1694.927 | 0 | −0.6 | 2116 | 420.9 | 420.9 | 420.9 | 6.46 |
| 843 | R.TRVPQFSFLDIFPK.V | | 565.647 | 3 | 1694.925 | 1694.927 | 0 | −0.7 | 2116 | 363.0 | 363.0 | 363.0 | 6.35 |
| 843 | R.TRVPQFSFLDIFPK.V | | 565.647 | 3 | 1694.927 | 1694.927 | 0 | 0.3 | 2116 | 340.4 | 340.4 | 340.4 | 5.98 |
| 843 | R.TRVPQFSFLDIFPK.V | | 565.647 | 3 | 1694.927 | 1694.927 | 0 | 0.4 | 2116 | 324.7 | 324.7 | 324.7 | 5.98 |
| 843 | R.TRVPQFSFLDIFPK.V | | 847.967 | 2 | 1694.927 | 1694.927 | 0 | 0.3 | 2116 | 308.7 | 270.3 | 270.3 | 5.21 |
| 830 | R.VPQFSFLDIFPK.V | | 719.393 | 2 | 1437.779 | 1437.778 | 0 | 0.8 | 2118 | 581.6 | 581.6 | 581.6 | 9.58 |
| 830 | R.VPQFSFLDIFPK.V | | 719.393 | 2 | 1437.779 | 1437.778 | 0 | 1.0 | 2118 | 403.5 | 403.5 | 403.5 | 7.86 |
| 830 | R.VPQFSFLDIFPK.V | | 719.393 | 2 | 1437.778 | 1437.778 | 0 | 0.1 | 2118 | 423.2 | 423.2 | 423.2 | 7.64 |
| 830 | R.VPQFSFLDIFPK.V | | 479.931 | 3 | 1437.778 | 1437.778 | 0 | 0.2 | 2118 | 454.7 | 454.7 | 454.7 | 7.26 |
| 830 | R.VPQFSFLDIFPK.V | | 479.931 | 3 | 1437.777 | 1437.778 | 0 | −0.2 | 2118 | 431.7 | 431.7 | 431.7 | 7.10 |
| 830 | R.VPQFSFLDIFPK.V | | 719.394 | 2 | 1437.780 | 1437.778 | 0 | 1.6 | 2118 | 317.5 | 317.5 | 317.5 | 5.97 |
| 830 | R.VPQFSFLDIFPK.V | | 719.393 | 2 | 1437.779 | 1437.778 | 0 | 1.1 | 2118 | 318.6 | 318.6 | 318.6 | 5.76 |
| 1691 | K.VTC[+57.021]RPPKEVIDMELSALR.S | C[+57] | 705.375 | 3 | 2114.109 | 2114.110 | 0 | −0.2 | 2130 | 554.5 | 554.5 | 554.5 | 8.86 |
| 1691 | K.VTC[+57.021]RPPKEVIDMELSALR.S | C[+57 | 529.283 | 4 | 2114.110 | 2114.110 | 0 | 0.0 | 2130 | 346.1 | 346.1 | 346.1 | 6.19 |
| 1691 | K.VTC[+57.021]RPPKEVIDMELSALR.S | C[+57] | 529.283 | 4 | 2114.110 | 2114.110 | 0 | 0.0 | 2130 | 330.3 | 330.3 | 330.3 | 5.99 |
| 1740 | R.SDTEPGMDLWEFC[+57.021]SETFQRPYQYLR.R | C[+57] | 789.604 | 4 | 3155.392 | 3155.387 | 0 | 1.6 | 2148 | 601.8 | 596.6 | 596.6 | 9.70 |
| 1740 | R.SDTEPGMDLWEFC[+57.021]SETFQRPYQYLR.R | C[+57] | 1052.469 | 3 | 3155.393 | 3155.387 | 0 | 1.6 | 2148 | 555.1 | 555.1 | 555.1 | 8.57 |
| 1740 | R.SDTEPGMDLWEFC[+57.021]SETFQRPYQYLR.R | C[+57] | 789.604 | 4 | 3155.392 | 3155.387 | 0 | 1.6 | 2148 | 525.5 | 525.5 | 525.5 | 8.03 |
| 3308 | R.SDTEPGMDLWEFC[+57.021]SETFQRPYQYLRR.F | C[+57] | 828.628 | 4 | 3311.491 | 3311.489 | 0 | 0.7 | 2148 | 326.1 | 267.5 | 267.5 | 5.23 |
| 1773 | R.FLNYQLR.D | | 477.264 | 2 | 953.521 | 953.520 | 0 | 0.2 | 2221 | 433.5 | 433.5 | 433.5 | 7.26 |
| 1773 | R.FLNYQLR.D | | 477.264 | 2 | 953.520 | 953.520 | 0 | 0.0 | 2221 | 370.7 | 370.7 | 370.7 | 7.14 |
| 1773 | R.FLNYQLR.D | | 477.264 | 2 | 953.521 | 953.520 | 0 | 0.2 | 2221 | 341.8 | 341.8 | 341.8 | 5.58 |
| 1773 | R.FLNYQLR.D | | 477.264 | 2 | 953.520 | 953.520 | 0 | 0.0 | 2221 | 341.2 | 341.2 | 341.2 | 5.58 |
| 1773 | R.FLNYQLR.D | | 477.264 | 2 | 953.520 | 953.520 | 0 | 0.1 | 2221 | 368.5 | 253.8 | 253.8 | 5.39 |
| 1773 | R.FLNYQLR.D | | 477.264 | 2 | 953.520 | 953.520 | 0 | 0.0 | 2221 | 347.3 | 225.7 | 225.7 | 5.32 |
| 3202 | K.HMVTM[+15.995]DGVREEDLAPFSLR.K | M[+16] | 740.358 | 3 | 2219.058 | 2219.058 | 0 | −0.1 | 2277 | 477.5 | 477.5 | 276.8 | 8.52 |
| 1883 | K.HMVTMDGVREEDLAPFSLRK.R | | 583.545 | 4 | 2331.158 | 2331.158 | 0 | 0.0 | 2277 | 405.5 | 405.5 | 405.5 | 7.47 |
| 1088 | K.HMVTMDGVREEDLAPFSLR.K | | 1102.035 | 2 | 2203.063 | 2203.063 | 0 | −0.1 | 2277 | 420.8 | 420.8 | 420.8 | 6.47 |
| 1883 | K.HMVTMDGVREEDLAPFSLRK.R | | 467.038 | 5 | 2331.158 | 2331.158 | 0 | 0.0 | 2277 | 356.8 | 356.8 | 356.8 | 5.85 |
| 1088 | K.HMVTMDGVREEDLAPFSLR.K | | 1102.035 | 2 | 2203.063 | 2203.063 | 0 | −0.4 | 2277 | 333.7 | 333.7 | 333.7 | 5.07 |
| 1121 | R.EEDLAPFSLRK.R | | 435.566 | 3 | 1304.685 | 1304.685 | 0 | 0.1 | 2286 | 579.5 | 579.5 | 579.5 | 9.32 |
| 930 | R.EEDLAPFSLR.K | | 588.798 | 2 | 1176.589 | 1176.590 | 0 | −0.2 | 2286 | 471.9 | 471.9 | 471.9 | 7.80 |
| 930 | R.EEDLAPFSLR.K | | 588.799 | 2 | 1176.590 | 1176.590 | 0 | 0.6 | 2286 | 426.4 | 420.4 | 420.4 | 7.64 |
| 1121 | R.EEDLAPFSLRK.R | | 652.846 | 2 | 1304.685 | 1304.685 | 0 | 0.3 | 2286 | 475.3 | 475.3 | 475.3 | 7.54 |
| 3179 | R.KRWESEPHPYVFFNDDHTTM[+15.995]TFIGFHLQPNINGS | M[+16] | 729.357 | 7 | 5099.458 | 5099.453 | 0 | 0.9 | 2296 | 371.0 | 371.0 | 371.0 | 6.03 |
| | VDAISHLTGK.V | | | | | | | | | | | | |
| 1186 | K.RWESEPHPYVFFNDDHTTMTFIGFHLQPNINGSVDAISHLTGK. | | 708.773 | 7 | 4955.369 | 4955.364 | 0 | 1.1 | 2297 | 530.0 | 530.0 | 530.0 | 7.29 |
| | V | | | | | | | | | | | | |
| 1186 | K.RWESEPHPYVFFNDDHTTMTFIGFHLQPNINGSVDAISHLTGK. | | 708.773 | 7 | 4955.369 | 4955.364 | 0 | 1.1 | 2297 | 483.7 | 483.7 | 483.7 | 7.15 |
| | V | | | | | | | | | | | | |
| 840 | R.WESEPHPYVFFNDDHTTMTFIGFHLQPNINGSVDAISHLTGK.V | | 960.659 | 5 | 4799.265 | 4799.262 | 0 | 0.5 | 2298 | 876.5 | 876.5 | 876.5 | 14.72 |
| 840 | R.WESEPHPYVFFNDDHTTMTFIGFHLQPNINGSVDAISHLTGK.V | | 960.659 | 5 | 4799.265 | 4799.262 | 0 | 0.6 | 2298 | 838.7 | 838.7 | 838.7 | 13.73 |
| 840 | R.WESEPHPYVFFNDDHTTMTFIGFHLQPNINGSVDAISHLTGK.V | | 800.717 | 6 | 4799.264 | 4799.262 | 0 | 0.4 | 2298 | 687.9 | 687.9 | 687.9 | 10.73 |
| 840 | R.WESEPHPYVFFNDDHTTMTFIGFHLQPNINGSVDAISHLTGK.V | | 800.717 | 6 | 4799.263 | 4799.262 | 0 | 0.2 | 2298 | 623.1 | 623.1 | 623.1 | 9.73 |
| 840 | R.WESEPHPYVFFNDDHTTMTFIGFHLQPNINGSVDAISHLTGK.V | | 800.881 | 6 | 4800.248 | 4799.262 | 1 | −3.8 | 2298 | 635.0 | 612.1 | 8.8 | 7.66 |
| 3120 | R.WESEPHPYVFFNDDHTTMTFIGFHLQPN[+0.984]INGSVDAI | N[+1] | 800.884 | 6 | 4800.266 | 4800.246 | 0 | 4.0 | 2298 | 550.8 | 550.8 | 22.2 | 5.68 |
| | SHLTGK.V | | | | | | | | | | | | |
| 840 | R.WESEPHPYVFFNDDHTTMTFIGFHLQPNINGSVDAISHLTGK.V | | 960.656 | 5 | 4799.250 | 4799.262 | 0 | −2.6 | 2298 | 486.6 | 343.0 | 343.0 | 4.26 |
| 868 | R.DVMTRDLYQGLLLQR.V | | 607.661 | 3 | 1820.970 | 1820.969 | 0 | 0.5 | 2344 | 517.2 | 517.2 | 517.2 | 8.52 |
| 1068 | R.DLYQGLLLQRVPFNVDFDKLPR.H | | 882.818 | 3 | 2646.440 | 2646.440 | 0 | −0.1 | 2349 | 811.9 | 811.9 | 811.9 | 14.19 |
| 984 | R.DLYQGLLLQR.V | | 609.846 | 2 | 1218.684 | 1218.684 | 0 | 0.2 | 2349 | 612.6 | 612.6 | 612.6 | 10.58 |
| 984 | R.DLYQGLLLQR.V | | 609.846 | 2 | 1218.684 | 1218.684 | 0 | 0.2 | 2349 | 611.2 | 611.2 | 611.2 | 10.58 |
| 1068 | R.DLYQGLLLQRVPFNVDFDKLPR.H | | 882.819 | 3 | 2646.442 | 2646.440 | 0 | 0.6 | 2349 | 594.5 | 594.5 | 594.5 | 9.19 |
| 1068 | R.DLYQGLLLQRVPFNVDFDKLPR.H | | 662.366 | 4 | 2646.442 | 2646.440 | 0 | 0.5 | 2349 | 562.3 | 562.3 | 562.3 | 9.19 |
| 1068 | R.DLYQGLLLQRVPFNVDFDKLPR.H | | 530.094 | 5 | 2646.442 | 2646.440 | 0 | 0.5 | 2349 | 561.6 | 561.6 | 561.6 | 8.65 |
| 1068 | R.DLYQGLLLQRVPFNVDFDKLPR.H | | 662.366 | 4 | 2646.442 | 2646.440 | 0 | 0.5 | 2349 | 548.9 | 548.9 | 548.9 | 8.52 |
| 984 | R.DLYQGLLLQR.V | | 406.900 | 3 | 1218.684 | 1218.684 | 0 | 0.3 | 2349 | 478.2 | 478.2 | 478.2 | 8.03 |
| 984 | R.DLYQGLLLQR.V | | 406.900 | 3 | 1218.684 | 1218.684 | 0 | 0.0 | 2349 | 519.3 | 519.3 | 519.3 | 7.69 |
| 841 | R.VPFNVDFDKLPR.H | | 482.930 | 3 | 1446.774 | 1446.774 | 0 | −0.1 | 2359 | 605.7 | 605.7 | 605.7 | 10.32 |
| 841 | R.VPFNVDFDKLPR.H | | 482.930 | 3 | 1446.774 | 1446.774 | 0 | 0.0 | 2359 | 575.8 | 575.8 | 575.8 | 9.32 |
| 841 | R.VPFNVDFDKLPR.H | | 723.891 | 2 | 1446.774 | 1446.774 | 0 | 0.3 | 2359 | 538.1 | 538.1 | 538.1 | 8.65 |
| 841 | R.VPFNVDFDKLPR.H | | 723.891 | 2 | 1446.774 | 1446.774 | 0 | 0.1 | 2359 | 490.5 | 490.5 | 490.5 | 8.52 |
| 841 | R.VPFNVDFDKLPR.H | | 482.930 | 3 | 1446.774 | 1446.774 | 0 | −0.1 | 2359 | 521.5 | 521.5 | 521.5 | 8.10 |
| 841 | R.VPFNVDFDKLPR.H | | 723.891 | 2 | 1446.774 | 1446.774 | 0 | −0.1 | 2359 | 1348.1 | 348.1 | 348.1 | 5.98 |
| 841 | R.VPFNVDFDKLPR.H | | 482.929 | 3 | 1446.774 | 1446.774 | 0 | −0.3 | 2359 | 334.0 | 213.9 | 213.9 | 5.34 |
| 1748 | R.LC[+57.021]LTLGIPQATDPDKTYELTTDNMLK.I | C[+57] | 984.496 | 3 | 2951.475 | 2951.474 | 0 | 0.2 | 2377 | 620.4 | 620.4 | 620.4 | 10.53 |
| 1748 | R.LC[+57.021]LTLGIPQATDPDKTYELTTDNMLK.I | C[+57] | 738.624 | 4 | 2951.476 | 2951.474 | 0 | 0.5 | 2377 | 466.5 | 466.5 | 466.5 | 7.21 |
| 1798 | K.ILAIEMR.F | | 423.249 | 2 | 845.491 | 845.491 | 0 | 0.1 | 2403 | 467.5 | 467.5 | 467.5 | 7.42 |
| 1798 | K.ILAIEMR.F | | 423.249 | 2 | 845.491 | 845.491 | 0 | −0.2 | 2403 | 424.7 | 424.7 | 424.7 | 7.26 |
| 1570 | K.ILAIEM[+15.995]R.F | M[+16] | 431.247 | 2 | 861.486 | 861.486 | 0 | −0.4 | 2403 | 330.5 | 330.5 | 330.5 | 5.58 |
| 3975 | R.LIKFLSDLRR.G | | 420.931 | 3 | 1260.779 | 1260.779 | 0 | 0.0 | 2429 | 462.1 | 462.1 | 462.1 | 7.21 |
| 1781 | K.FLSDLRR.G | | 453.762 | 2 | 906.516 | 906.516 | 0 | 0.2 | 2432 | 378.2 | 265.9 | 265.9 | 5.13 |
| 2259 | R.GGTNADTIKLVK.V | | 608.848 | 2 | 1216.690 | 1216.690 | 0 | 0.0 | 2439 | 620.2 | 442.6 | 442.6 | 9.77 |
| 1506 | R.GGTN[+0.984]ADTIKLVK.V | N[+1] | 406.563 | 3 | 1217.674 | 1217.674 | 0 | 0.0 | 2439 | 474.1 | 351.9 | 351.9 | 6.59 |
| 866 | K.VHGGTTADMIYSR.V | | 704.338 | 2 | 1407.668 | 1407.669 | 0 | −0.2 | 2451 | 667.2 | 667.2 | 667.2 | 11.58 |
| 1538 | K.VHGGTTADM[+15.995]IYSR.V | M[+16] | 712.336 | 2 | 1423.664 | 1423.663 | 0 | 0.4 | 2451 | 603.6 | 603.6 | 603.6 | 10.58 |
| 1538 | K.VHGGTTADM[+15.995]IYSR.V | M[+16] | 475.226 | 3 | 1423.664 | 1423.663 | 0 | 0.2 | 2451 | 504.3 | 504.3 | 504.3 | 8.23 |
| 866 | K.VHGGTTADMIYSR.V | | 469.894 | 3 | 1407.669 | 1407.669 | 0 | 0.0 | 2451 | 471.5 | 471.5 | 471.5 | 8.02 |
| 866 | K.VHGGTTADMIYSR.V | | 469.894 | 3 | 1407.668 | 1407.669 | 0 | −0.2 | 2451 | 465.6 | 465.6 | 465.6 | 8.02 |
| 866 | K.VHGGTTADMIYSR.V | | 469.894 | 3 | 1407.668 | 1407.669 | 0 | −0.3 | 2451 | 452.8 | 452.8 | 452.8 | 8.02 |
| 866 | K.VHGGTTADMIYSR.V | | 704.338 | 2 | 1407.668 | 1407.669 | 0 | −0.2 | 2451 | 442.9 | 442.9 | 442.9 | 8.02 |
| 866 | K.VHGGTTADMIYSR.V | | 469.894 | 3 | 1407.668 | 1407.669 | 0 | −0.2 | 2451 | 424.9 | 424.9 | 424.9 | 7.86 |
| 866 | K.VHGGTTADMIYSR.V | | 469.894 | 3 | 1407.668 | 1407.669 | 0 | −0.3 | 2451 | 461.0 | 461.0 | 461.0 | 7.80 |
| 1538 | K.VHGGTTADM[+15.995]IYSR.V | M[+16] | 475.226 | 3 | 1423.664 | 1423.663 | 0 | 0.6 | 2451 | 458.9 | 458.9 | 458.9 | 7.80 |
| 1538 | K.VHGGTTADM[+15.995]IYSR.V | M[+16] | 475.226 | 3 | 1423.664 | 1423.663 | 0 | 0.2 | 2451 | 431.0 | 431.0 | 431.0 | 7.64 |
| 866 | K.VHGGTTADMIYSR.V | | 469.894 | 3 | 1407.668 | 1407.669 | 0 | −0.3 | 2451 | 404.7 | 404.7 | 404.7 | 7.64 |
| 1538 | K.VHGGTTADM[+15.995]IYSR.V | M[+16] | 475.226 | 3 | 1423.664 | 1423.663 | 0 | 0.2 | 2451 | 380.7 | 380.7 | 380.7 | 7.53 |
| 1538 | K.VHGGTTADM[+15.995]IYSR.V | M[+16] | 475.226 | 3 | 1423.664 | 1423.663 | 0 | 0.5 | 2451 | 416.6 | 416.6 | 416.6 | 7.44 |
| 866 | K.VHGGTTADMIYSR.V | | 469.895 | 3 | 1407.669 | 1407.669 | 0 | 0.2 | 2451 | 414.9 | 414.9 | 414.9 | 7.44 |
| 866 | K.VHGGTTADMIYSR.V | | 469.894 | 3 | 1407.668 | 1407.669 | 0 | −0.3 | 2451 | 386.6 | 386.6 | 386.6 | 7.32 |
| 1538 | K.VHGGTTADM[+15.995]IYSR.V | M[+16] | 475.226 | 3 | 1423.663 | 1423.663 | 0 | −0.2 | 2451 | 369.4 | 369.4 | 369.4 | 7.14 |
| 1538 | K.VHGGTTADM[+15.995]IYSR.V | M[+16] | 475.226 | 3 | 1423.663 | 1423.663 | 0 | −0.5 | 2451 | 377.6 | 377.6 | 377.6 | 6.22 |
| 866 | K.VHGGTTADMIYSR.V | | 469.895 | 3 | 1407.669 | 1407.669 | 0 | 0.4 | 2451 | 340.1 | 340.1 | 340.1 | 6.04 |
| 1538 | K.VHGGTTADM[+15.995]IYSR.V | M[+16] | 475.226 | 3 | 1423.664 | 1423.663 | 0 | 0.2 | 2451 | 348.0 | 348.0 | 348.0 | 5.86 |
| 866 | K.VHGGTTADMIYSR.V | | 469.895 | 3 | 1407.669 | 1407.669 | 0 | 0.4 | 2451 | 337.1 | 337.1 | 337.1 | 5.86 |
| 866 | K.VHGGTTADMIYSR.V | | 469.894 | 3 | 1407.668 | 1407.669 | 0 | −0.5 | 2451 | 338.2 | 338.2 | 338.2 | 5.12 |
| 1538 | K.VHGGTTADM[+15.995]IYSR.V | M[+16] | 475.226 | 3 | 1423.663 | 1423.663 | 0 | −0.4 | 2451 | 375.9 | 353.3 | 353.3 | 5.05 |
| 1538 | K.VHGGTTADM[+15.995]IYSR.V | M[+16] | 475.226 | 3 | 1423.663 | 1423.663 | 0 | −0.5 | 2451 | 312.0 | 312.0 | 312.0 | 4.39 |
| 866 | K.VHGGTTADMIYSR.V | | 704.334 | 2 | 1407.661 | 1407.669 | 0 | −5.2 | 2451 | 319.3 | 319.3 | 319.3 | 4.13 |
| 1926 | R.VREAENVAFANK.D | | 449.905 | 3 | 1347.702 | 1347.702 | 0 | 0.1 | 2464 | 431.2 | 431.2 | 431.2 | 7.38 |
| 890 | R.LESAGLGYR.V | | 483.256 | 2 | 965.505 | 965.505 | 0 | 0.1 | 2538 | 569.3 | 569.3 | 569.3 | 8.81 |
| 890 | R.LESAGLGYR.V | | 483.256 | 2 | 965.505 | 965.505 | 0 | −0.1 | 2538 | 550.5 | 550.5 | 550.5 | 8.14 |
| 890 | R.LESAGLGYR.V | | 483.256 | 2 | 965.505 | 965.505 | 0 | −0.1 | 2538 | 523.0 | 523.0 | 523.0 | 8.14 |
| 890 | R.LESAGLGYR.V | | 483.256 | 2 | 965.505 | 965.505 | 0 | −0.1 | 2538 | 515.2 | 515.2 | 515.2 | 8.01 |
| 890 | R.LESAGLGYR.V | | 483.256 | 2 | 965.505 | 965.505 | 0 | −0.2 | 2538 | 483.2 | 483.2 | 483.2 | 8.01 |
| 890 | R.LESAGLGYR.V | | 483.256 | 2 | 965.505 | 965.505 | 0 | −0.1 | 2538 | 484.9 | 484.9 | 484.9 | 7.80 |
| 890 | R.LESAGLGYR.V | | 483.256 | 2 | 965.505 | 965.505 | 0 | −0.2 | 2538 | 400.8 | 400.8 | 400.8 | 7.44 |
| 890 | R.LESAGLGYR.V | | 483.256 | 2 | 965.505 | 965.505 | 0 | −0.2 | 2538 | 397.0 | 397.0 | 397.0 | 7.14 |
| 890 | R.LESAGLGYR.V | | 483.256 | 2 | 965.505 | 965.505 | 0 | −0.1 | 2538 | 307.6 | 307.6 | 307.6 | 5.31 |
| 1190 | R.VSMEETADRLGSIPLR.Q | | 591.977 | 3 | 1773.917 | 1773.916 | 0 | 0.4 | 2547 | 608.8 | 608.8 | 608.8 | 10.32 |
| 1190 | R.VSMEETADRLGSIPLR.Q | | 591.977 | 3 | 1773.916 | 1773.916 | 0 | −0.1 | 2547 | 515.9 | 515.9 | 515.9 | 7.97 |
| 1190 | R.VSMEETADRLGSIPLR.Q | | 591.977 | 3 | 1773.917 | 1773.916 | 0 | 0.3 | 2547 | 493.3 | 493.3 | 493.3 | 7.97 |
| 1587 | R.VSM[+15.995]EETADRLGSIPLR.Q | M[+16] | 597.309 | 3 | 1789.911 | 1789.911 | 0 | 0.0 | 2547 | 464.7 | 464.7 | 464.7 | 7.54 |
| 1587 | R.VSM[+15.995]EETADRLGSIPLR.Q | M[+16] | 597.308 | 3 | 1789.910 | 1789.911 | 0 | −0.5 | 2547 | 471.8 | 471.8 | 471.8 | 6.85 |
| 3537 | R.VSMEETADRLGSIPLRQLVYR.V | | 811.769 | 3 | 2433.293 | 2433.292 | 0 | 0.4 | 2547 | 315.9 | 315.9 | 315.9 | 5.19 |
| 1547 | R.VSM[+15.995]EETADR.L | M[+16] | 527.229 | 2 | 1053.451 | 1053.452 | 0 | −0.6 | 2547 | 345.5 | 345.5 | 345.5 | 4.66 |
| 2512 | R.LGSIPLRQLVYR.V | | 472.289 | 3 | 1414.853 | 1414.853 | 0 | −0.2 | 2556 | 551.9 | 551.9 | 551.9 | 8.10 |
| 2512 | R.LGSIPLRQLVYR.V | | 472.289 | 3 | 1414.852 | 1414.853 | 0 | −0.4 | 2556 | 420.6 | 420.6 | 420.6 | 7.60 |
| 1234 | R.VHALPPSLIPLVWDFGQLSDVAEKLYIQQIVQR.L | | 755.422 | 5 | 3773.083 | 3773.079 | 0 | 1.1 | 2568 | 749.6 | 749.6 | 749.6 | 11.32 |
| 1234 | R.VHALPPSLIPLVWDFGQLSDVAEKLYIQQIVQR.L | | 755.422 | 5 | 3773.079 | 3773.079 | 0 | 0.1 | 2568 | 705.1 | 705.1 | 705.1 | 11.32 |
| 1234 | R.VHALPPSLIPLVWDFGQLSDVAEKLYIQQIVQR.L | | 944.027 | 4 | 3773.084 | 3773.079 | 0 | 1.4 | 2568 | 642.5 | 642.5 | 642.5 | 9.85 |
| 981 | K.LYIQQIVQR.L | | 580.843 | 2 | 1160.678 | 1160.679 | 0 | −0.2 | 2592 | 540.5 | 540.5 | 540.5 | 8.36 |
| 981 | K.LYIQQIVQR.L | | 580.843 | 2 | 1160.678 | 1160.679 | 0 | −0.2 | 2592 | 448.3 | 448.3 | 448.3 | 8.02 |
| 2006 | R.LVESISLDENGTR.V | | 716.867 | 2 | 1432.727 | 1432.728 | 0 | −0.4 | 2601 | 531.9 | 531.9 | 531.9 | 7.99 |
| 2006 | R.LVESISLDENGTR.V | | 478.582 | 3 | 1433.730 | 1432.728 | 1 | −0.6 | 2601 | 405.3 | 358.1 | 358.1 | 3.44 |
| 883 | R.FFPKPYDDSRLLLDEITR.A | | 742.392 | 3 | 2225.160 | 2225.160 | 0 | −0.1 | 2710 | 489.7 | 489.7 | 489.7 | 8.72 |
| 883 | R.FFPKPYDDSRLLLDEITR.A | | 742.392 | 3 | 2225.160 | 2225.160 | 0 | −0.1 | 2710 | 448.8 | 448.8 | 448.8 | 8.52 |
| 883 | R.FFPKPYDDSRLLLDEITR.A | | 557.046 | 4 | 2225.160 | 2225.160 | 0 | 0.0 | 2710 | 425.3 | 425.3 | 425.3 | 8.36 |
| 883 | R.FFPKPYDDSRLLLDEITR.A | | 557.045 | 4 | 2225.160 | 2225.160 | 0 | −0.1 | 2710 | 416.7 | 416.7 | 416.7 | 8.36 |
| 938 | R.FFPKPYDDSR.L | | 636.306 | 2 | 1271.605 | 1271.606 | 0 | −0.1 | 2710 | 486.0 | 486.0 | 486.0 | 7.80 |
| 938 | R.FFPKPYDDSR.L | | 636.307 | 2 | 1271.606 | 1271.606 | 0 | 0.2 | 2710 | 439.0 | 439.0 | 439.0 | 7.44 |
| 938 | R.FFPKPYDDSR.L | | 424.540 | 3 | 1271.605 | 1271.606 | 0 | −0.5 | 2710 | 370.2 | 370.2 | 370.2 | 6.22 |
| 883 | R.FFPKPYDDSRLLLDEITR.A | | 557.297 | 4 | 2226.164 | 2225.160 | 1 | 0.3 | 2710 | 354.7 | 354.7 | 354.7 | 6.19 |
| 938 | R.FFPKPYDDSR.L | | 424.540 | 3 | 1271.606 | 1271.606 | 0 | 0.1 | 2710 | 358.6 | 358.6 | 358.6 | 6.04 |
| 938 | R.FFPKPYDDSR.L | | 424.540 | 3 | 1271.606 | 1271.606 | 0 | 0.2 | 2710 | 346.3 | 346.3 | 346.3 | 5.86 |
| 938 | R.FFPKPYDDSR.L | | 424.540 | 3 | 1271.606 | 1271.606 | 0 | 0.2 | 2710 | 335.2 | 335.2 | 335.2 | 5.86 |
| 938 | R.FFPKPYDDSR.L | | 424.540 | 3 | 1271.605 | 1271.606 | 0 | −0.1 | 2710 | 333.9 | 333.9 | 333.9 | 5.86 |
| 938 | R.FFPKPYDDSR.L | | 424.540 | 3 | 1271.605 | 1271.606 | 0 | −0.5 | 2710 | 339.0 | 339.0 | 339.0 | 5.58 |
| 902 | R.LLLDEITRAQDLFLDGVPLRK.T | | 809.132 | 3 | 2425.383 | 2425.381 | 0 | 0.6 | 2720 | 719.5 | 719.5 | 719.5 | 12.19 |
| 1256 | R.LLLDEITRAQDLFLDGVPLR.K | | 766.769 | 3 | 2298.291 | 2297.286 | 1 | 0.7 | 2720 | 518.8 | 518.8 | 518.8 | 8.72 |
| 902 | R.LLLDEITRAQDLFLDGVPLRK.T | | 607.101 | 4 | 2425.382 | 2425.381 | 0 | 0.1 | 2720 | 408.6 | 408.6 | 408.6 | 7.47 |
| 2028 | R.LLLDEITR.A | | 486.790 | 2 | 972.573 | 972.572 | 0 | 0.2 | 2720 | 450.2 | 450.2 | 450.2 | 7.42 |
| 902 | R.LLLDEITRAQDLFLDGVPLRK.T | | 607.101 | 4 | 2425.382 | 2425.381 | 0 | 0.3 | 2720 | 382.0 | 382.0 | 382.0 | 7.36 |
| 2028 | R.LLLDEITR.A | | 486.790 | 2 | 972.572 | 972.572 | 0 | −0.2 | 2720 | 361.0 | 361.0 | 361.0 | 7.32 |
| 2028 | R.LLLDEITR.A | | 486.790 | 2 | 972.573 | 972.572 | 0 | 0.4 | 2720 | 398.4 | 398.4 | 398.4 | 7.14 |
| 1256 | R.LLLDEITRAQDLFLDGVPLR.K | | 766.434 | 3 | 2297.288 | 2297.286 | 0 | 0.6 | 2720 | 350.2 | 350.2 | 350.2 | 6.19 |
| 902 | R.LLLDEITRAQDLFLDGVPLRK.T | | 809.133 | 3 | 2425.383 | 2425.381 | 0 | 0.6 | 2720 | 348.5 | 348.5 | 348.5 | 5.47 |
| 902 | R.LLLDEITRAQDLFLDGVPLRK.T | | 485.882 | 5 | 2425.382 | 2425.381 | 0 | 0.3 | 2720 | 341.0 | 341.0 | 341.0 | 5.31 |
| 922 | R.AQDLFLDGVPLRK.T | | 491.280 | 3 | 1471.827 | 1471.827 | 0 | −0.1 | 2728 | 641.8 | 641.8 | 641.8 | 10.32 |
| 922 | R.AQDLFLDGVPLRK.T | | 491.280 | 3 | 1471.827 | 1471.827 | 0 | −0.2 | 2728 | 613.2 | 613.2 | 613.2 | 10.32 |
| 1189 | R.AQDLFLDGVPLR.K | | 672.370 | 2 | 1343.732 | 1343.732 | 0 | 0.1 | 2728 | 584.0 | 584.0 | 584.0 | 9.58 |
| 1189 | R.AQDLFLDGVPLR.K | | 672.369 | 2 | 1343.732 | 1343.732 | 0 | −0.1 | 2728 | 577.6 | 577.6 | 577.6 | 9.58 |
| 922 | R.AQDLFLDGVPLRK.T | | 491.280 | 3 | 1471.827 | 1471.827 | 0 | −0.1 | 2728 | 598.1 | 598.1 | 598.1 | 9.32 |
| 922 | R.AQDLFLDGVPLRK.T | | 736.417 | 2 | 1471.826 | 1471.827 | 0 | −0.4 | 2728 | 596.9 | 596.9 | 596.9 | 9.32 |
| 922 | R.AQDLFLDGVPLRK.T | | 491.280 | 3 | 1471.827 | 1471.827 | 0 | −0.1 | 2728 | 574.5 | 574.5 | 574.5 | 9.32 |
| 922 | R.AQDLFLDGVPLRK.T | | 736.417 | 2 | 1471.827 | 1471.827 | 0 | −0.1 | 2728 | 540.1 | 540.1 | 540.1 | 8.65 |
| 922 | R.AQDLFLDGVPLRK.T | | 736.417 | 2 | 1471.827 | 1471.827 | 0 | −0.1 | 2728 | 507.0 | 507.0 | 507.0 | 8.52 |
| 922 | R.AQDLFLDGVPLRK.T | | 736.417 | 2 | 1471.827 | 1471.827 | 0 | −0.2 | 2728 | 369.4 | 369.4 | 369.4 | 7.27 |
| 922 | R.AQDLFLDGVPLRK.T | | 491.280 | 3 | 1471.827 | 1471.827 | 0 | −0.1 | 2728 | 332.6 | 332.6 | 332.6 | 5.98 |
| 2639 | K.IPLFLVGKPGSSK.S | | 448.275 | 3 | 1342.809 | 1342.809 | 0 | −0.2 | 2763 | 425.1 | 425.1 | 425.1 | 7.64 |
| 850 | K.TIVADAMQGPAAYSDLFR.S | | 963.475 | 2 | 1925.943 | 1925.943 | 0 | 0.3 | 2780 | 603.7 | 603.7 | 603.7 | 10.79 |
| 850 | K.TIVADAMQGPAAYSDLFR.S | | 963.477 | 2 | 1925.947 | 1925.943 | 0 | 2.3 | 2780 | 611.6 | 611.6 | 611.6 | 9.49 |
| 850 | K.TIVADAMQGPAAYSDLFR.S | | 642.653 | 3 | 1925.943 | 1925.943 | 0 | 0.4 | 2780 | 599.7 | 599.7 | 599.7 | 9.25 |
| 850 | K.TIVADAMQGPAAYSDLFR.S | | 642.653 | 3 | 1925.944 | 1925.943 | 0 | 0.5 | 2780 | 565.4 | 565.4 | 565.4 | 9.25 |
| 850 | K.TIVADAMQGPAAYSDLFR.S | | 642.652 | 3 | 1925.943 | 1925.943 | 0 | 0.1 | 2780 | 537.7 | 537.7 | 537.7 | 8.58 |
| 850 | K.TIVADAMQGPAAYSDLFR.S | | 642.652 | 3 | 1925.943 | 1925.943 | 0 | 0.1 | 2780 | 496.4 | 496.4 | 496.4 | 8.45 |
| 850 | K.TIVADAMQGPAAYSDLFR.S | | 963.473 | 2 | 1925.938 | 1925.943 | 0 | −2.2 | 2780 | 528.4 | 528.4 | 528.4 | 8.20 |
| 850 | K.TIVADAMQGPAAYSDLFR.S | | 642.653 | 3 | 1925.945 | 1925.943 | 0 | 1.0 | 2780 | 494.7 | 494.7 | 494.7 | 7.90 |
| 1545 | K.TIVADAM[+15.995]QGPAAYSDLFR.S | M[+16] | 647.984 | 3 | 1941.938 | 1941.938 | 0 | 0.3 | 2780 | 432.4 | 432.4 | 432.4 | 7.31 |
| 850 | K.TIVADAMQGPAAYSDLFR.S | | 642.654 | 3 | 1925.948 | 1925.943 | 0 | 2.7 | 2780 | 542.6 | 542.6 | 542.6 | 7.28 |
| 850 | K.TIVADAMQGPAAYSDLFR.S | | 642.652 | 3 | 1925.943 | 1925.943 | 0 | 0.1 | 2780 | 386.0 | 386.0 | 386.0 | 7.20 |
| 850 | K.TIVADAMQGPAAYSDLFR.S | | 642.653 | 3 | 1925.944 | 1925.943 | 0 | 0.5 | 2780 | 349.0 | 349.0 | 349.0 | 5.91 |
| 1688 | R.SLKQVHLVSFQC[+57.021]SPHSTPQGIISTFR.Q | C[+57] | 739.388 | 4 | 2954.531 | 2954.531 | 0 | 0.1 | 2798 | 770.2 | 770.2 | 770.2 | 13.53 |
| 1688 | R.SLKQVHLVSFQC[+57.021]SPHSTPQGIISTFR.Q | C[+57] | 591.712 | 5 | 2954.529 | 2954.531 | 0 | −0.7 | 2798 | 306.8 | 273.0 | 273.0 | 4.22 |
| 1416 | R.FQQGKDLQQYVSVVVLDEVGLAEDSPKMPLK.T | | 1154.274 | 3 | 3460.807 | 3460.803 | 0 | 1.2 | 2828 | 833.8 | 833.8 | 833.8 | 13.52 |
| 1416 | R.FQQGKDLQQYVSVVVLDEVGLAEDSPKMPLK.T | | 865.956 | 4 | 3460.804 | 3460.803 | 0 | 0.2 | 2828 | 733.7 | 733.7 | 733.7 | 11.52 |
| 1416 | R.FQQGKDLQQYVSVVVLDEVGLAEDSPKMPLK.T | | 865.956 | 4 | 3460.803 | 3460.803 | 0 | −0.1 | 2828 | 711.5 | 711.5 | 711.5 | 11.52 |
| 1416 | R.FQQGKDLQQYVSVVVLDEVGLAEDSPKMPLK.T | | 1154.274 | 3 | 3460.807 | 3460.803 | 0 | 1.2 | 2828 | 699.9 | 699.9 | 699.9 | 10.52 |
| 1617 | R.FQQGKDLQQYVSVVVLDEVGLAEDSPKM[+15.995]PLK.T | M[+16] | 1159.606 | 3 | 3476.804 | 3476.798 | 0 | 1.5 | 2828 | 629.2 | 626.6 | 626.6 | 8.97 |
| 1416 | R.FQQGKDLQQYVSVVVLDEVGLAEDSPKMPLK.T | | 692.967 | 5 | 3460.805 | 3460.803 | 0 | 0.6 | 2828 | 591.1 | 591.1 | 591.1 | 7.98 |
| 1617 | R.FQQGKDLQQYVSVVVLDEVGLAEDSPKM[+15.995]PLK.T | M[+16] | 696.166 | 5 | 3476.801 | 3476.798 | 0 | 0.7 | 2828 | 597.8 | 597.8 | 597.8 | 7.43 |
| 1416 | R.FQQGKDLQQYVSVVVLDEVGLAEDSPKMPLK.T | | 692.967 | 5 | 3460.804 | 3460.803 | 0 | 0.3 | 2828 | 537.5 | 537.5 | 537.5 | 7.31 |
| 961 | R.FQQGKDLQQYVSVVVLDEVGLAEDSPK.M | | 997.849 | 3 | 2991.533 | 2991.531 | 0 | 0.8 | 2828 | 430.6 | 430.6 | 430.6 | 7.21 |
| 1617 | R.FQQGKDLQQYVSVVVLDEVGLAEDSPKM[+15.995]PLK.T | M[+16] | 869.956 | 4 | 3476.804 | 3476.798 | 0 | 1.6 | 2828 | 485.3 | 485.3 | 485.3 | 6.94 |
| 961 | R.FQQGKDLQQYVSVVVLDEVGLAEDSPK.M | | 748.639 | 4 | 2991.533 | 2991.531 | 0 | 0.5 | 2828 | 368.8 | 368.8 | 368.8 | 6.73 |
| 961 | R.FQQGKDLQQYVSVVVLDEVGLAEDSPK.M | | 997.851 | 3 | 2991.538 | 2991.531 | 0 | 2.4 | 2828 | 448.0 | 448.0 | 448.0 | 6.45 |
| 961 | R.FQQGKDLQQYVSVVVLDEVGLAEDSPK.M | | 997.850 | 3 | 2991.536 | 2991.531 | 0 | 1.7 | 2828 | 347.4 | 347.4 | 347.4 | 5.81 |
| 961 | R.FQQGKDLQQYVSVVVLDEVGLAEDSPK.M | | 748.639 | 4 | 2991.533 | 2991.531 | 0 | 0.7 | 2828 | 312.7 | 312.7 | 312.7 | 5.17 |
| 1416 | R.FQQGKDLQQYVSVVVLDEVGLAEDSPKMPLK.T | | 577.640 | 6 | 3460.802 | 3460.803 | 0 | −0.4 | 2828 | 386.5 | 386.5 | 386.5 | 4.80 |
| 1416 | R.FQQGKDLQQYVSVVVLDEVGLAEDSPKMPLK.T | | 865.957 | 4 | 3460.806 | 3460.803 | 0 | 0.7 | 2828 | 353.2 | 205.3 | 205.3 | 4.26 |
| 1171 | K.DLQQYVSVVVLDEVGLAEDSPKMPLK.T | | 718.881 | 4 | 2872.504 | 2872.501 | 0 | 0.8 | 2833 | 818.1 | 818.1 | 818.1 | 13.99 |
| 1171 | K.DLQQYVSVVVLDEVGLAEDSPKMPLK.T | | 718.882 | 4 | 2872.505 | 2872.501 | 0 | 1.2 | 2833 | 713.9 | 713.9 | 713.9 | 11.99 |
| 1171 | K.DLQQYVSVVVLDEVGLAEDSPKMPLK.T | | 958.174 | 3 | 2872.507 | 2872.501 | 0 | 1.9 | 2833 | 737.2 | 737.2 | 737.2 | 11.23 |
| 932 | K.DLQQYVSVVVLDEVGLAEDSPK.M | | 1202.119 | 2 | 2403.231 | 2403.229 | 0 | 0.7 | 2833 | 620.5 | 576.6 | 576.6 | 10.79 |
| 1171 | K.DLQQYVSVVVLDEVGLAEDSPKMPLK.T | | 958.173 | 3 | 2872.505 | 2872.501 | 0 | 1.2 | 2833 | 607.3 | 553.3 | 553.3 | 10.53 |
| 1171 | K.DLQQYVSVVVLDEVGLAEDSPKMPLK.T | | 718.881 | 4 | 2872.504 | 2872.501 | 0 | 0.9 | 2833 | 614.3 | 591.5 | 591.5 | 9.99 |
| 932 | K.DLQQYVSVVVLDEVGLAEDSPK.M | | 1202.118 | 2 | 2403.229 | 2403.229 | 0 | 0.2 | 2833 | 456.6 | 456.6 | 456.6 | 8.78 |
| 932 | K.DLQQYVSVVVLDEVGLAEDSPK.M | | 801.748 | 3 | 2403.231 | 2403.229 | 0 | 0.7 | 2833 | 544.5 | 544.5 | 544.5 | 8.58 |
| 1546 | K.DLQQYVSVVVLDEVGLAEDSPKM[+15.995]PLK.T | M[+16] | 722.880 | 4 | 2888.497 | 2888.496 | 0 | 0.3 | 2833 | 550.8 | 550.8 | 550.8 | 8.32 |
| 1171 | K.DLQQYVSVVVLDEVGLAEDSPKMPLK.T | | 1436.758 | 2 | 2872.509 | 2872.501 | 0 | 2.8 | 2833 | 599.0 | 599.0 | 599.0 | 8.23 |
| 932 | K.DLQQYVSVVVLDEVGLAEDSPK.M | | 801.748 | 3 | 2403.230 | 2403.229 | 0 | 0.6 | 2833 | 413.0 | 413.0 | 413.0 | 7.53 |
| 932 | K.DLQQYVSVVVLDEVGLAEDSPK.M | | 801.748 | 3 | 2403.229 | 2403.229 | 0 | 0.1 | 2833 | 381.4 | 288.3 | 288.3 | 5.45 |
| 932 | K.DLQQYVSVVVLDEVGLAEDSPK.M | | 802.084 | 3 | 2404.237 | 2403.229 | 1 | 2.1 | 2833 | 326.6 | 309.1 | 309.1 | 4.05 |
| 1686 | K.MPLKTLHPLLEDGC[+57.021]IEDDPAPHKK.V | C[+57] | 689.354 | 4 | 2754.393 | 2754.395 | 0 | −0.8 | 2855 | 666.9 | 666.9 | 666.9 | 8.93 |
| 1686 | K.MPLKTLHPLLEDGC[+57.021]IEDDPAPHKK.V | C[+57] | 551.685 | 5 | 2754.396 | 2754.395 | 0 | 0.2 | 2855 | 550.5 | 550.5 | 550.5 | 8.52 |
| 1686 | K.MPLKTLHPLLEDGC[+57.021]IEDDPAPHKK.V | CI+57 | 459.905 | 6 | 2754.395 | 2754.395 | 0 | 0.1 | 2855 | 321.4 | 321.4 | 321.4 | 5.65 |
| 1723 | K.TLHPLLEDGC[+57.021]IEDDPAPHKK.V | C[+57] | 572.036 | 4 | 2285.122 | 2285.123 | 0 | −0.3 | 2859 | 790.1 | 790.1 | 790.1 | 13.53 |
| 1723 | K.TLHPLLEDGC[+57.021]IEDDPAPHKK.V | C[+57] | 572.036 | 4 | 2285.122 | 2285.123 | 0 | −0.3 | 2859 | 735.7 | 735.7 | 735.7 | 12.53 |
| 1723 | K.TLHPLLEDGC[+57.021]IEDDPAPHKK.V | C[+57] | 572.036 | 4 | 2285.123 | 2285.123 | 0 | 0.1 | 2859 | 725.9 | 725.9 | 725.9 | 12.53 |
| 1723 | K.TLHPLLEDGC[+57.021]IEDDPAPHKK.V | C[+57] | 762.379 | 3 | 2285.123 | 2285.123 | 0 | 0.0 | 2859 | 798.9 | 798.9 | 798.9 | 12.19 |
| 1723 | K.TLHPLLEDGC[+57.021]IEDDPAPHKK.V | C[+57] | 762.378 | 3 | 2285.121 | 2285.123 | 0 | −1.1 | 2859 | 761.6 | 761.6 | 761.6 | 11.27 |
| 1723 | K.TLHPLLEDGC[+57.021]IEDDPAPHKK.V | C[+57] | 457.831 | 5 | 2285.124 | 2285.123 | 0 | 0.2 | 2859 | 595.7 | 595.7 | 595.7 | 9.53 |
| 1723 | K.TLHPLLEDGC[+57.021]IEDDPAPHKK.V | C[+57 | 572.287 | 4 | 2286.126 | 2285.123 | 1 | −0.3 | 2859 | 581.2 | 581.2 | 581.2 | 9.53 |
| 1723 | K.TLHPLLEDGC[+57.021]IEDDPAPHKK.V | C[+57 | 457.830 | 5 | 2285.123 | 2285.123 | 0 | −0.1 | 2859 | 537.1 | 537.1 | 537.1 | 8.86 |
| 4119 | K.KVGFVGISNWALDPAKMNR.G | | 526.785 | 4 | 2104.120 | 2103.117 | 1 | −0.3 | 2878 | 478.7 | 478.7 | 68.5 | 7.97 |
| 1966 | K.KVGFVGISNWALDPAK.M | | 567.982 | 3 | 1701.933 | 1701.932 | 0 | 0.3 | 2878 | 409.3 | 385.6 | 385.6 | 7.64 |
| 1006 | K.VGFVGISNWALDPAKMNR.G | | 659.347 | 3 | 1976.025 | 1975.022 | 1 | −0.1 | 2879 | 670.7 | 670.7 | 259.6 | 11.53 |
| 1006 | K.VGFVGISNWALDPAKMNR.G | | 659.013 | 3 | 1975.024 | 1975.022 | 0 | 0.9 | 2879 | 660.1 | 660.1 | 660.1 | 11.53 |
| 1006 | K.VGFVGISNWALDPAKMNR.G | | 659.013 | 3 | 1975.024 | 1975.022 | 0 | 1.1 | 2879 | 651.5 | 651.5 | 651.5 | 11.53 |
| 836 | K.VGFVGISNWALDPAK.M | | 787.422 | 2 | 1573.837 | 1573.837 | 0 | −0.3 | 2879 | 646.4 | 646.4 | 646.4 | 10.58 |
| 1006 | K.VGFVGISNWALDPAKMNR.G | | 659.013 | 3 | 1975.023 | 1975.022 | 0 | 0.6 | 2879 | 604.9 | 604.9 | 604.9 | 10.53 |
| 1006 | K.VGFVGISNWALDPAKMNR.G | | 659.012 | 3 | 1975.023 | 1975.022 | 0 | 0.4 | 2879 | 602.2 | 602.2 | 602.2 | 10.53 |
| 1006 | K.VGFVGISNWALDPAKMNR.G | | 659.012 | 3 | 1975.023 | 1975.022 | 0 | 0.4 | 2879 | 1585.6 | 576.5 | 576.5 | 9.53 |
| 1530 | K.VGFVGISNWALDPAKM[+15.995]NR.G | M[+16] | 664.344 | 3 | 1991.018 | 1991.017 | 0 | 0.5 | 2879 | 1559.4 | 559.4 | 559.4 | 8.86 |
| 1006 | K.VGFVGISNWALDPAKMNR.G | | 659.013 | 3 | 1975.024 | 1975.022 | 0 | 1.0 | 2879 | 555.9 | 555.9 | 555.9 | 8.86 |
| 1530 | K.VGFVGISNWALDPAKM[+15.995]NR.G | M[+16] | 996.012 | 2 | 1991.017 | 1991.017 | 0 | −0.1 | 2879 | 530.4 | 530.4 | 530.4 | 8.86 |
| 836 | K.VGFVGISNWALDPAK.M | | 787.422 | 2 | 1573.837 | 1573.837 | 0 | −0.1 | 2879 | 464.7 | 464.7 | 464.7 | 8.57 |
| 836 | K.VGFVGISNWALDPAK.M | | 787.422 | 2 | 1573.837 | 1573.837 | 0 | −0.2 | 2879 | 428.2 | 428.2 | 428.2 | 8.41 |
| 836 | K.VGFVGISNWALDPAK.M | | 787.422 | 2 | 1573.837 | 1573.837 | 0 | −0.1 | 2879 | 469.4 | 469.4 | 469.4 | 8.02 |
| 836 | K.VGFVGISNWALDPAK.M | | 525.284 | 3 | 1573.837 | 1573.837 | 0 | 0.0 | 2879 | 452.8 | 452.8 | 452.8 | 7.26 |
| 1006 | K.VGFVGISNWALDPAKMNR.G | | 659.013 | 3 | 1975.024 | 1975.022 | 0 | 1.3 | 2879 | 347.0 | 347.0 | 347.0 | 6.19 |
| 1737 | R.GSPNETELIESAKGIC[+57.021]SSDILVQDR.V | C[+57] | 906.780 | 3 | 2718.326 | 2718.325 | 0 | 0.5 | 2903 | 725.0 | 725.0 | 725.0 | 12.53 |
| 990 | R.GSPNETELIESAK.G | | 687.841 | 2 | 1374.675 | 1374.675 | 0 | 0.1 | 2903 | 663.2 | 663.2 | 663.2 | 11.58 |
| 990 | R.GSPNETELIESAK.G | | 687.841 | 2 | 1374.675 | 1374.675 | 0 | −0.1 | 2903 | 601.7 | 601.7 | 601.7 | 10.58 |
| 990 | R.GSPNETELIESAK.G | | 458.896 | 3 | 1374.675 | 1374.675 | 0 | −0.1 | 2903 | 506.5 | 506.5 | 506.5 | 8.24 |
| 990 | R.GSPNETELIESAK.G | | 458.897 | 3 | 1374.675 | 1374.675 | 0 | 0.1 | 2903 | 466.2 | 465.9 | 465.9 | 7.26 |
| 1479 | R.GSPN[+0.984]ETELIESAK.G | N[+1] | 688.333 | 2 | 1375.660 | 1375.659 | 0 | 0.5 | 2903 | 366.5 | 366.5 | 366.5 | 6.87 |
| 3340 | K.GIC[+57.021]SSDILVQDR.V | C[+57] | 681.838 | 2 | 1362.668 | 1362.668 | 0 | −0.2 | 2916 | 439.6 | 439.6 | 439.6 | 7.86 |
| 1862 | R.VQGYFASFAK.A | | 559.288 | 2 | 1117.568 | 1117.568 | 0 | 0.0 | 2928 | 468.7 | 468.7 | 468.7 | 7.80 |
| 877 | K.RQDKEFFGLR.D | | 432.567 | 3 | 1295.685 | 1295.686 | 0 | −0.1 | 2945 | 397.9 | 397.9 | 397.9 | 7.06 |
| 877 | K.RQDKEFFGLR.D | | 432.567 | 3 | 1295.685 | 1295.686 | 0 | −0.4 | 2945 | 345.4 | 345.4 | 345.4 | 4.40 |
| 2123 | R.QDKEFFGLRDYYSLIK.M | | 506.264 | 4 | 2022.034 | 2022.033 | 0 | 0.3 | 2946 | 399.5 | 399.5 | 399.5 | 7.15 |
| 2041 | R.DYYSLIK.M | | 451.237 | 2 | 901.467 | 901.467 | 0 | 0.0 | 2955 | 358.5 | 358.5 | 358.5 | 5.58 |
| 1007 | K.ASNRKPSPQDIAQAVLR.N | | 617.678 | 3 | 1851.020 | 1851.020 | 0 | 0.5 | 2969 | 659.7 | 659.7 | 659.7 | 11.53 |
| 1007 | K.ASNRKPSPQDIAQAVLR.N | | 617.678 | 3 | 1851.021 | 1851.020 | 0 | 0.6 | 2969 | 561.3 | 561.3 | 561.3 | 9.53 |
| 1007 | K.ASNRKPSPQDIAQAVLR.N | | 617.678 | 3 | 1851.020 | 1851.020 | 0 | 0.3 | 2969 | 402.4 | 402.4 | 402.4 | 7.59 |
| 1007 | K.ASNRKPSPQDIAQAVLR.N | | 617.678 | 3 | 1851.020 | 1851.020 | 0 | 0.4 | 2969 | 345.1 | 345.1 | 345.1 | 6.19 |
| 839 | R.KPSPQDIAQAVLR.N | | 474.940 | 3 | 1422.806 | 1422.806 | 0 | 0.0 | 2973 | 390.0 | 390.0 | 390.0 | 7.75 |
| 839 | R.KPSPQDIAQAVLR.N | | 711.907 | 2 | 1422.807 | 1422.806 | 0 | 0.5 | 2973 | 380.5 | 380.5 | 380.5 | 7.75 |
| 839 | R.KPSPQDIAQAVLR.N | | 474.940 | 3 | 1422.806 | 1422.806 | 0 | −0.2 | 2973 | 436.4 | 436.4 | 436.4 | 7.64 |
| 839 | R.KPSPQDIAQAVLR.N | | 474.940 | 3 | 1422.807 | 1422.806 | 0 | 0.1 | 2973 | 376.1 | 376.1 | 376.1 | 7.32 |
| 839 | R.KPSPQDIAQAVLR.N | | 474.940 | 3 | 1422.806 | 1422.806 | 0 | 0.0 | 2973 | 371.5 | 371.5 | 371.5 | 7.32 |
| 839 | R.KPSPQDIAQAVLR.N | | 474.940 | 3 | 1422.806 | 1422.806 | 0 | 0.0 | 2973 | 362.5 | 362.5 | 362.5 | 7.14 |
| 839 | R.KPSPQDIAQAVLR.N | | 474.940 | 3 | 1422.807 | 1422.806 | 0 | 0.1 | 2973 | 337.0 | 337.0 | 337.0 | 6.04 |
| 815 | R.NFSGKDDIQALDIFLANLPEAK.C | | 1210.129 | 2 | 2419.251 | 2419.250 | 0 | 0.2 | 2986 | 476.5 | 438.8 | 438.8 | 8.52 |
| 815 | R.NFSGKDDIQALDIFLANLPEAK.C | | 807.089 | 3 | 2419.252 | 2419.250 | 0 | 0.5 | 2986 | 453.1 | 453.1 | 453.1 | 8.52 |
| 815 | R.NFSGKDDIQALDIFLANLPEAK.C | | 1210.129 | 2 | 2419.250 | 2419.250 | 0 | −0.1 | 2986 | 427.9 | 427.9 | 427.9 | 8.36 |
| 815 | R.NFSGKDDIQALDIFLANLPEAK.C | | 807.089 | 3 | 2419.254 | 2419.250 | 0 | 1.3 | 2986 | 489.1 | 489.1 | 489.1 | 8.18 |
| 815 | R.NFSGKDDIQALDIFLANLPEAK.C | | 807.088 | 3 | 2419.251 | 2419.250 | 0 | 0.1 | 2986 | 420.0 | 420.0 | 420.0 | 7.81 |
| 815 | R.NFSGKDDIQALDIFLANLPEAK.C | | 807.089 | 3 | 2419.252 | 2419.250 | 0 | 0.5 | 2986 | 418.0 | 418.0 | 418.0 | 7.59 |
| 815 | R.NFSGKDDIQALDIFLANLPEAK.C | | 807.089 | 3 | 2419.253 | 2419.250 | 0 | 1.2 | 2986 | 414.3 | 414.3 | 414.3 | 7.21 |
| 815 | R.NFSGKDDIQALDIFLANLPEAK.C | | 807.088 | 3 | 2419.251 | 2419.250 | 0 | 0.1 | 2986 | 342.5 | 342.5 | 342.5 | 5.99 |
| 815 | R.NFSGKDDIQALDIFLANLPEAK.C | | 807.089 | 3 | 2419.254 | 2419.250 | 0 | 1.3 | 2986 | 331.1 | 331.1 | 331.1 | 5.81 |
| 1490 | R.N[+0.984]FSGKDDIQALDIFLANLPEAK.C | N[+1] | 807.417 | 3 | 2420.235 | 2420.234 | 0 | 0.2 | 2986 | 325.5 | 325.5 | 219.2 | 5.81 |
| 815 | R.NFSGKDDIQALDIFLANLPEAK.C | | 605.568 | 4 | 2419.250 | 2419.250 | 0 | −0.4 | 2986 | 316.0 | 316.0 | 316.0 | 5.60 |
| 815 | R.NFSGKDDIQALDIFLANLPEAK.C | | 605.568 | 4 | 2419.251 | 2419.250 | 0 | 0.2 | 2986 | 320.6 | 320.6 | 320.6 | 5.27 |
| 815 | R.NFSGKDDIQALDIFLANLPEAK.C | | 605.568 | 4 | 2419.248 | 2419.250 | 0 | −0.9 | 2986 | 308.6 | 308.6 | 308.6 | 4.46 |
| 876 | K.DDIQALDIFLANLPEAK.C | | 629.335 | 3 | 1885.991 | 1885.991 | 0 | 0.2 | 2991 | 463.2 | 463.2 | 463.2 | 7.69 |
| 1717 | K.C[+57.021]SEEVSPMQLIK.Q | C[+57] | 710.844 | 2 | 1420.681 | 1420.681 | 0 | −0.2 | 3008 | 525.8 | 525.8 | 525.8 | 8.91 |
| 1671 | K.C[+57.021]SEEVSPM[+15.995]QLIK.Q | C[+57], | 718.842 | 2 | 1436.676 | 1436.676 | 0 | 0.2 | 3008 | 521.4 | 311.9 | 311.9 | 7.00 |
| | | M[+16] | | | | | | | | | | | |
| 1671 | K.C[+57.021]SEEVSPM[+15.995]QLIK.Q | C[+57], | 718.842 | 2 | 1436.676 | 1436.676 | 0 | 0.0 | 3008 | 458.2 | 257.0 | 257.0 | 6.33 |
| | | M[+16] | | | | | | | | | | | |
| 1166 | K.QNIFGPSQKVPGGEQEDAESR.Y | | 758.364 | 3 | 2273.079 | 2273.079 | 0 | −0.3 | 3020 | 853.0 | 853.0 | 853.0 | 15.48 |
| 1166 | K.QNIFGPSQKVPGGEQEDAESR.Y | | 758.365 | 3 | 2273.079 | 2273.079 | 0 | 0.1 | 3020 | 837.3 | 837.3 | 837.3 | 14.54 |
| 1166 | K.QNIFGPSQKVPGGEQEDAESR.Y | | 758.364 | 3 | 2273.078 | 2273.079 | 0 | −0.4 | 3020 | 701.3 | 701.3 | 701.3 | 11.61 |
| 940 | K.QNIFGPSQKVPGGEQEDAESRYLLVLTK.N | | 776.657 | 4 | 3103.606 | 3103.606 | 0 | 0.2 | 3020 | 616.0 | 616.0 | 616.0 | 10.19 |
| 940 | K.QNIFGPSQKVPGGEQEDAESRYLLVLTK.N | | 1035.207 | 3 | 3103.606 | 3103.606 | 0 | −0.1 | 3020 | 570.2 | 570.2 | 570.2 | 9.19 |
| 940 | K.QNIFGPSQKVPGGEQEDAESRYLLVLTK.N | | 776.657 | 4 | 3103.606 | 3103.606 | 0 | 0.0 | 3020 | 485.5 | 485.5 | 485.5 | 8.39 |
| 1950 | R.YLLVLTK.N | | 425.276 | 2 | 849.545 | 849.544 | 0 | 0.1 | 3041 | 364.3 | 364.3 | 364.3 | 6.87 |
| 1950 | R.YLLVLTK.N | | 425.276 | 2 | 849.545 | 849.544 | 0 | 0.1 | 3041 | 335.4 | 335.4 | 335.4 | 5.86 |
| 1732 | K.NYVALQILQQTFFEGDQQPEIIFGSGFPKDQEYTQLC. | C[+57] | 1127.804 | 4 | 4508.195 | 4508.187 | 0 | 1.9 | 3048 | 961.0 | 961.0 | 961.0 | 15.00 |
| | [+57.021]RN | | | | | | | | | | | | |
| 1732 | K.NYVALQILQQTFFEGDQQPEIIFGSGFPKDQEYTQLC. | C[+57] | 902.444 | 5 | 4508.193 | 4508.187 | 0 | 1.4 | 3048 | 767.2 | 767.2 | 767.2 | 12.32 |
| | [+57.021]RN | | | | | | | | | | | | |
| 957 | K.NYVALQILQQTFFEGDQQPEIIFGSGFPK.D | | 829.424 | 4 | 3314.675 | 3314.673 | 0 | 0.6 | 3048 | 664.5 | 664.5 | 664.5 | 10.58 |
| 957 | K.NYVALQILQQTFFEGDQQPEIIFGSGFPK.D | | 829.425 | 4 | 3314.678 | 3314.673 | 0 | 1.4 | 3048 | 632.3 | 632.3 | 632.3 | 9.58 |
| 957 | K.NYVALQILQQTFFEGDQQPEIIFGSGFPK.D | | 1105.565 | 3 | 3314.679 | 3314.673 | 0 | 1.7 | 3048 | 665.8 | 665.8 | 665.8 | 9.28 |
| 957 | K.NYVALQILQQTFFEGDQQPEIIFGSGFPK.D | | 1105.564 | 3 | 3314.677 | 3314.673 | 0 | 1.2 | 3048 | 397.7 | 397.7 | 397.7 | 5.86 |
| 1693 | K.DQEYTQLC[+57.021]R.N | C[+57] | 606.770 | 2 | 1212.532 | 1212.531 | 0 | 0.3 | 3077 | 480.3 | 480.3 | 480.3 | 7.80 |
| 1693 | K.DQEYTQLC[+57.021]R.N | C[+57] | 606.769 | 2 | 1212.531 | 1212.531 | 0 | −0.1 | 3077 | 426.0 | 426.0 | 426.0 | 7.44 |
| 1093 | K.YVDLGLGTHR.V | | 565.801 | 2 | 1130.595 | 1130.595 | 0 | −0.6 | 3128 | 559.9 | 559.9 | 559.9 | 7.45 |
| 1806 | R.LIVIEEK.D | | 422.263 | 2 | 843.519 | 843.519 | 0 | 0.0 | 3148 | 372.0 | 372.0 | 372.0 | 7.14 |
| 1806 | R.LIVIEEK.D | | 422.263 | 2 | 843.519 | 843.519 | 0 | 0.0 | 3148 | 351.2 | 351.2 | 351.2 | 5.86 |
| 4389 | K.HYLDINTVLEKWQK.S | | 596.321 | 3 | 1786.949 | 1786.949 | 0 | 0.1 | 3172 | 633.6 | 562.8 | 562.8 | 10.32 |
| 2000 | K.HYLDINTVLEK.W | | 448.910 | 3 | 1344.715 | 1344.716 | 0 | −0.3 | 3172 | 309.9 | 309.9 | 309.9 | 5.58 |
| 872 | R.ALTEELHQKVSEEAK.S | | 856.446 | 2 | 1711.884 | 1711.886 | 0 | −1.2 | 3241 | 800.2 | 800.2 | 800.2 | 12.06 |
| 872 | R.ALTEELHQKVSEEAK.S | | 571.300 | 3 | 1711.886 | 1711.886 | 0 | −0.2 | 3241 | 591.4 | 586.3 | 586.3 | 9.32 |
| 872 | R.ALTEELHQKVSEEAK.S | | 571.300 | 3 | 1711.886 | 1711.886 | 0 | −0.1 | 3241 | 580.9 | 580.9 | 580.9 | 9.32 |
| 872 | R.ALTEELHQKVSEEAK.S | | 428.727 | 4 | 1711.886 | 1711.886 | 0 | 0.0 | 3241 | 570.0 | 570.0 | 570.0 | 9.32 |
| 872 | R.ALTEELHQKVSEEAK.S | | 428.727 | 4 | 1711.886 | 1711.886 | 0 | −0.2 | 3241 | 512.1 | 484.1 | 484.1 | 8.52 |
| 872 | R.ALTEELHQKVSEEAK.S | | 571.300 | 3 | 1711.886 | 1711.886 | 0 | 0.0 | 3241 | 479.1 | 479.1 | 479.1 | 8.31 |
| 872 | R.ALTEELHQKVSEEAK.S | | 571.300 | 3 | 1711.885 | 1711.886 | 0 | −0.6 | 3241 | 531.9 | 531.9 | 531.9 | 7.73 |
| 872 | R.ALTEELHQKVSEEAK.S | | 856.447 | 2 | 1711.887 | 1711.886 | 0 | 0.3 | 3241 | 529.3 | 529.3 | 529.3 | 7.31 |
| 872 | R.ALTEELHQKVSEEAK.S | | 856.948 | 2 | 1712.889 | 1711.886 | 1 | −0.5 | 3241 | 561.2 | 561.2 | 561.2 | 7.06 |
| 872 | R.ALTEELHQKVSEEAK.S | | 571.300 | 3 | 1711.886 | 1711.886 | 0 | 0.0 | 3241 | 413.1 | 352.6 | 352.6 | 6.97 |
| 872 | R.ALTEELHQKVSEEAK.S | | 428.727 | 4 | 1711.886 | 1711.886 | 0 | 0.0 | 3241 | 425.5 | 341.4 | 341.4 | 6.20 |
| 872 | R.ALTEELHQKVSEEAK.S | | 856.446 | 2 | 1711.885 | 1711.886 | 0 | −0.6 | 3241 | 384.5 | 384.5 | 384.5 | 5.77 |
| 872 | R.ALTEELHQKVSEEAK.S | | 428.727 | 4 | 1711.887 | 1711.886 | 0 | 0.5 | 3241 | 302.9 | 302.9 | 302.9 | 5.50 |
| 872 | R.ALTEELHQKVSEEAK.S | | 428.727 | 4 | 1711.887 | 1711.886 | 0 | 0.4 | 3241 | 308.0 | 308.0 | 308.0 | 5.32 |
| 1695 | K.SILLNC[+57.021]ATPDAVVR.L | C[+57] | 764.912 | 2 | 1528.816 | 1528.815 | 0 | 0.7 | 3256 | 627.1 | 528.7 | 528.7 | 10.58 |
| 1695 | K.SILLNC[+57.021]ATPDAVVR.L | C[+57] | 510.277 | 3 | 1528.816 | 1528.815 | 0 | 0.4 | 3256 | 432.3 | 338.8 | 338.8 | 5.92 |
| 1173 | R.LSAYSLGGFAAEWLSQEYFHR.Q | | 811.395 | 3 | 2432.171 | 2432.167 | 0 | 1.5 | 3270 | 784.7 | 784.7 | 784.7 | 13.79 |
| 1173 | R.LSAYSLGGFAAEWLSQEYFHR.Q | | 608.798 | 4 | 2432.169 | 2432.167 | 0 | 0.7 | 3270 | 763.1 | 763.1 | 763.1 | 13.25 |
| 1173 | R.LSAYSLGGFAAEWLSQEYFHR.Q | | 1216.589 | 2 | 2432.171 | 2432.167 | 0 | 1.6 | 3270 | 550.5 | 550.5 | 550.5 | 9.12 |
| 1173 | R.LSAYSLGGFAAEWLSQEYFHR.Q | | 811.394 | 3 | 2432.166 | 2432.167 | 0 | −0.2 | 3270 | 407.7 | 407.7 | 407.7 | 7.85 |
| 1005 | R.QRHNSFADFLQAHLHTADLER.H | | 627.314 | 4 | 2506.234 | 2506.233 | 0 | 0.4 | 3291 | 682.8 | 682.8 | 682.8 | 10.19 |
| 1005 | R.QRHNSFADFLQAHLHTADLER.H | | 836.082 | 3 | 2506.233 | 2506.233 | 0 | −0.4 | 3291 | 662.2 | 662.2 | 662.2 | 10.19 |
| 1005 | R.QRHNSFADFLQAHLHTADLER.H | | 502.053 | 5 | 2506.234 | 2506.233 | 0 | 0.3 | 3291 | 503.2 | 503.2 | 503.2 | 8.72 |
| 1005 | R.QRHNSFADFLQAHLHTADLER.H | | 502.053 | 5 | 2506.234 | 2506.233 | 0 | 0.3 | 3291 | 431.0 | 431.0 | 431.0 | 7.81 |
| 1005 | R.QRHNSFADFLQAHLHTADLER.H | | 418.879 | 6 | 2508.240 | 2506.233 | 2 | 0.0 | 3291 | 429.3 | 429.3 | 75.9 | 6.58 |
| 1005 | R.QRHNSFADFLQAHLHTADLER.H | | 627.314 | 4 | 2506.233 | 2506.233 | 0 | −0.2 | 3291 | 381.5 | 381.2 | 381.2 | 5.93 |
| 854 | R.HNSFADFLQAHLHTADLER.H | | 556.274 | 4 | 2222.073 | 2222.074 | 0 | −0.3 | 3293 | 695.4 | 695.4 | 695.4 | 11.79 |
| 854 | R.HNSFADFLQAHLHTADLER.H | | 556.274 | 4 | 2222.073 | 2222.074 | 0 | −0.2 | 3293 | 660.3 | 660.3 | 660.3 | 11.79 |
| 854 | R.HNSFADFLQAHLHTADLER.H | | 556.274 | 4 | 2222.074 | 2222.074 | 0 | −0.1 | 3293 | 635.2 | 635.2 | 635.2 | 10.79 |
| 854 | R.HNSFADFLQAHLHTADLER.H | | 556.274 | 4 | 2222.074 | 2222.074 | 0 | 0.2 | 3293 | 620.0 | 620.0 | 620.0 | 10.79 |
| 854 | R.HNSFADFLQAHLHTADLER.H | | 445.221 | 5 | 2222.074 | 2222.074 | 0 | 0.1 | 3293 | 575.3 | 575.3 | 575.3 | 9.79 |
| 854 | R.HNSFADFLQAHLHTADLER.H | | 445.221 | 5 | 2222.074 | 2222.074 | 0 | 0.2 | 3293 | 429.5 | 429.5 | 429.5 | 8.62 |
| 854 | R.HNSFADFLQAHLHTADLER.H | | 741.360 | 3 | 2222.067 | 2222.074 | 0 | −3.1 | 3293 | 478.3 | 478.3 | 478.3 | 5.37 |
| 943 | R.HAIFTEITTFSR.L | | 711.873 | 2 | 1422.738 | 1422.738 | 0 | 0.1 | 3312 | 523.2 | 523.2 | 523.2 | 8.91 |
| 943 | R.HAIFTEITTFSR.L | | 711.873 | 2 | 1422.739 | 1422.738 | 0 | 0.6 | 3312 | 508.2 | 508.2 | 508.2 | 8.78 |
| 943 | R.HAIFTEITTFSR.L | | 711.873 | 2 | 1422.738 | 1422.738 | 0 | 0.3 | 3312 | 483.1 | 483.1 | 483.1 | 8.78 |
| 943 | R.HAIFTEITTFSR.L | | 711.873 | 2 | 1422.738 | 1422.738 | 0 | 0.1 | 3312 | 396.2 | 396.2 | 396.2 | 8.30 |
| 943 | R.HAIFTEITTFSR.L | | 474.917 | 3 | 1422.737 | 1422.738 | 0 | −0.2 | 3312 | 384.9 | 384.9 | 384.9 | 7.75 |
| 943 | R.HAIFTEITTFSR.L | | 474.917 | 3 | 1422.738 | 1422.738 | 0 | 0.0 | 3312 | 393.4 | 393.4 | 393.4 | 7.53 |
| 943 | R.HAIFTEITTFSR.L | | 474.917 | 3 | 1422.738 | 1422.738 | 0 | 0.0 | 3312 | 384.6 | 384.6 | 384.6 | 7.32 |
| 943 | R.HAIFTEITTFSR.L | | 474.917 | 3 | 1422.737 | 1422.738 | 0 | −0.7 | 3312 | 421.4 | 421.4 | 421.4 | 6.94 |
| 1143 | R.APKPTLLWLQQFDTEYSFLKEVR.N | | 937.169 | 3 | 2809.492 | 2809.492 | 0 | −0.1 | 3341 | 851.7 | 851.7 | 851.7 | 15.48 |
| 1143 | R.APKPTLLWLQQFDTEYSFLKEVR.N | | 703.129 | 4 | 2809.493 | 2809.492 | 0 | 0.4 | 3341 | 611.1 | 611.1 | 611.1 | 10.53 |
| 1143 | R.APKPTLLWLQQFDTEYSFLKEVR.N | | 562.905 | 5 | 2810.497 | 2809.492 | 1 | 0.3 | 3341 | 585.3 | 585.3 | 585.3 | 8.99 |
| 1143 | R.APKPTLLWLQQFDTEYSFLKEVR.N | | 703.128 | 4 | 2809.491 | 2809.492 | 0 | −0.5 | 3341 | 586.0 | 586.0 | 586.0 | 8.61 |
| 894 | R.APKPTLLWLQQFDTEYSFLK.E | | 809.099 | 3 | 2425.283 | 2425.280 | 0 | 1.2 | 3341 | 516.5 | 516.5 | 516.5 | 8.01 |
| 894 | R.APKPTLLWLQQFDTEYSFLK.E | | 607.076 | 4 | 2425.282 | 2425.280 | 0 | 0.6 | 3341 | 425.6 | 425.6 | 425.6 | 7.31 |
| 1143 | R.APKPTLLWLQQFDTEYSFLKEVR.N | | 703.129 | 4 | 2809.492 | 2809.492 | 0 | −0.1 | 3341 | 340.1 | 340.1 | 340.1 | 5.99 |
| 2170 | K.ILIFQTDFEDGIR.S | | 783.912 | 2 | 1566.816 | 1566.816 | 0 | −0.1 | 3374 | 483.8 | 483.8 | 483.8 | 8.78 |
| 858 | K.ILIFQTDFEDGIRSAQLIASAK.Y | | 812.777 | 3 | 2436.315 | 2436.313 | 0 | 0.7 | 3374 | 519.9 | 519.9 | 519.9 | 8.72 |
| 2170 | K.ILIFQTDFEDGIR.S | | 783.912 | 2 | 1566.817 | 1566.816 | 0 | 0.5 | 3374 | 462.8 | 462.8 | 462.8 | 8.57 |
| 858 | K.ILIFQTDFEDGIRSAQLIASAK.Y | | 609.834 | 4 | 2436.315 | 2436.313 | 0 | 0.9 | 3374 | 505.7 | 505.7 | 505.7 | 8.19 |
| 2170 | K.ILIFQTDFEDGIR.S | | 783.912 | 2 | 1566.817 | 1566.816 | 0 | 0.5 | 3374 | 367.8 | 367.8 | 367.8 | 7.32 |
| 2170 | K.ILIFQTDFEDGIR.S | | 522.943 | 3 | 1566.815 | 1566.816 | 0 | −0.7 | 3374 | 390.1 | 390.1 | 390.1 | 6.07 |
| 3530 | R.SAQLIASAK.Y | | 444.761 | 2 | 888.515 | 888.515 | 0 | 0.0 | 3387 | 370.0 | 370.0 | 370.0 | 7.32 |
| 3530 | R.SAQLIASAK.Y | | 444.761 | 2 | 888.515 | 888.515 | 0 | 0.2 | 3387 | 339.8 | 339.8 | 339.8 | 6.04 |
| 848 | K.YSVINEINK.I | | 540.290 | 2 | 1079.573 | 1079.573 | 0 | 0.0 | 3396 | 525.5 | 525.5 | 525.5 | 8.14 |
| 848 | K.YSVINEINK.I | | 540.290 | 2 | 1079.573 | 1079.573 | 0 | 0.0 | 3396 | 307.7 | 307.7 | 307.7 | 5.76 |
| 855 | R.RSTLMVSDVTR.L | | 422.227 | 3 | 1264.668 | 1264.668 | 0 | −0.2 | 3447 | 482.6 | 429.6 | 429.6 | 8.01 |
| 855 | R.RSTLMVSDVTR.L | | 422.228 | 3 | 1264.668 | 1264.668 | 0 | 0.1 | 3447 | 406.7 | 406.7 | 406.7 | 7.26 |
| 1581 | R.RSTLM[+15.995]VSDVTR.L | M[+16] | 427.559 | 3 | 1280.664 | 1280.663 | 0 | 0.7 | 3447 | 352.3 | 352.3 | 352.3 | 5.86 |
| 1242 | R.STLMVSDVTRLQHVTISQLFAPGDLPELGLEHR.A | | 915.737 | 4 | 3659.925 | 3659.921 | 0 | 0.9 | 3448 | 718.2 | 718.2 | 718.2 | 11.85 |
| 1242 | R.STLMVSDVTRLQHVTISQLFAPGDLPELGLEHR.A | | 732.791 | 5 | 3659.924 | 3659.921 | 0 | 0.8 | 3448 | 637.5 | 637.5 | 637.5 | 9.85 |
| 845 | R.STLMVSDVTR.L | | 554.787 | 2 | 1108.567 | 1108.567 | 0 | −0.1 | 3448 | 510.7 | 510.7 | 510.7 | 8.23 |
| 845 | R.STLMVSDVTR.L | | 554.787 | 2 | 1108.567 | 1108.567 | 0 | 0.0 | 3448 | 530.1 | 530.1 | 530.1 | 8.14 |
| 1579 | R.STLM[+15.995]VSDVTR.L | M[+16] | 562.784 | 2 | 1124.561 | 1124.562 | 0 | −0.2 | 3448 | 457.3 | 457.3 | 457.3 | 8.02 |
| 1579 | R.STLM[+15.995]VSDVTR.L | M[+16] | 562.785 | 2 | 1124.562 | 1124.562 | 0 | 0.1 | 3448 | 446.8 | 446.8 | 446.8 | 7.60 |
| 1579 | R.STLM[+15.995]VSDVTR.L | M[+16] | 562.784 | 2 | 1124.561 | 1124.562 | 0 | −0.2 | 3448 | 439.0 | 439.0 | 439.0 | 7.44 |
| 1242 | R.STLMVSDVTRLQHVTISQLFAPGDLPELGLEHR.A | | 915.735 | 4 | 3659.919 | 3659.921 | 0 | −0.8 | 3448 | 511.3 | 511.3 | 511.3 | 6.58 |
| 845 | R.STLMVSDVTR.L | | 554.787 | 2 | 1108.567 | 1108.567 | 0 | 0.1 | 3448 | 311.7 | 311.7 | 311.7 | 5.58 |
| 979 | R.LQHVTISQLFAPGDLPELGLEHR.A | | 857.462 | 3 | 2570.372 | 2570.373 | 0 | −0.1 | 3458 | 737.8 | 737.8 | 737.8 | 12.79 |
| 979 | R.LQHVTISQLFAPGDLPELGLEHR.A | | 643.349 | 4 | 2570.372 | 2570.373 | 0 | −0.2 | 3458 | 691.1 | 691.1 | 691.1 | 11.79 |
| 1391 | R.LQHVTISQLFAPGDLPELGLEHRAEDGHEEAMETEASTSGEVA | | 1073.340 | 6 | 6435.002 | 6435.004 | 0 | −0.4 | 3458 | 693.0 | 693.0 | 693.0 | 9.65 |
| | EVAEEAMETESSEKVGK.E | | | | | | | | | | | | |
| 1391 | R.LQHVTISQLFAPGDLPELGLEHRAEDGHEEAMETEASTSGEVA | | 1073.674 | 6 | 6437.010 | 6435.004 | 2 | −0.1 | 3458 | 651.6 | 651.6 | 651.6 | 9.65 |
| | EVAEEAMETESSEKVGK.E | | | | | | | | | | | | |
| 979 | R.LQHVTISQLFAPGDLPELGLEHR.A | | 514.880 | 5 | 2570.373 | 2570.373 | 0 | 0.2 | 3458 | 542.1 | 542.1 | 542.1 | 8.58 |
| 1391 | R.LQHVTISQLFAPGDLPELGLEHRAEDGHEEAMETEASTSGEVA | | 1073.507 | 6 | 6436.006 | 6435.004 | 1 | −0.3 | 3458 | 593.3 | 593.3 | 593.3 | 7.65 |
| | EVAEEAMETESSEKVGK.E | | | | | | | | | | | | |
| 835 | R.LQHVTISQLFAPGDLPELGLEHRAEDGHEEAMETEASTSGEVA | | 879.552 | 7 | 6150.819 | 6150.819 | 0 | −0.1 | 3458 | 593.1 | 593.1 | 593.1 | 7.45 |
| | EVAEEAMETESSEK.V | | | | | | | | | | | | |
| 1391 | R.LQHVTISQLFAPGDLPELGLEHRAEDGHEEAMETEASTSGEVA | | 920.149 | 7 | 6435.002 | 6435.004 | 0 | −0.3 | 3458 | 528.1 | 528.1 | 528.1 | 6.43 |
| | EVAEEAMETESSEKVGK.E | | | | | | | | | | | | |
| 1532 | R.LQHVTISQLFAPGDLPELGLEHRAEDGHEEAMETEASTSGEVA | M[+16] | 881.837 | 7 | 6166.817 | 6166.814 | 0 | 0.5 | 3458 | 465.4 | 465.4 | 60.9 | 5.29 |
| | EVAEEAM[+15.995]ETESSEK.V | | | | | | | | | | | | |
| 1835 | R.LQHVTISQLFAPGDLPELGLEHRAEDGHEEAMETEASTSGEVA | | 1231.372 | 5 | 6152.830 | 6150.819 | 2 | 0.7 | 3458 | 387.1 | 387.1 | 387.1 | 5.15 |
| | EVAEEAMETESSEK.V | | | | | | | | | | | | |
| 835 | R.LQHVTISQLFAPGDLPELGLEHRAEDGHEEAMETEASTSGEVA | | 879.696 | 7 | 6151.829 | 6150.819 | 1 | 1.0 | 3458 | 409.7 | 409.7 | 409.7 | 4.85 |
| | EVAEEAMETESSEK.V | | | | | | | | | | | | |
| 835 | R.LQHVTISQLFAPGDLPELGLEHRAEDGHEEAMETEASTSGEVA | | 879.553 | 7 | 6150.825 | 6150.819 | 0 | 0.9 | 3458 | 463.6 | 358.3 | 358.3 | 4.11 |
| | EVAEEAMETESSEK.V | | | | | | | | | | | | |
| 835 | R.LQHVTISQLFAPGDLPELGLEHRAEDGHEEAMETEASTSGEVA | | 879.552 | 7 | 6150.819 | 6150.819 | 0 | 0.0 | 3458 | 383.6 | 285.9 | 285.9 | 3.65 |
| | EVAEEAMETESSEK.V | | | | | | | | | | | | |
| 954 | R.AEDGHEEAMETEASTSGEVAEVAEEAMETESSEKVGKETSEL | | 1129.904 | 5 | 5645.492 | 5644.492 | 1 | −0.6 | 3481 | 845.9 | 845.9 | 845.9 | 11.73 |
| | GGSDVSILDTTR.L | | | | | | | | | | | | |
| 3260 | R.AEDGHEEAM[+15.995]ETEASTSGEVAEVAEEAMETESSEKV | M[+16] | 1132.903 | 5 | 5660.487 | 5660.487 | 0 | 0.0 | 3481 | 781.9 | 781.9 | 397.6 | 11.65 |
| | GKETSELGGSDVSILDTTR.L | | | | | | | | | | | | |
| 948 | R.AEDGHEEAMETEASTSGEVAEVAEEAMETESSEK.V | | 1200.493 | 3 | 3599.464 | 3599.464 | 0 | −0.1 | 3481 | 697.3 | 697.3 | 697.3 | 11.11 |
| 948 | R.AEDGHEEAMETEASTSGEVAEVAEEAMETESSEK.V | | 900.621 | 4 | 3599.462 | 3599.464 | 0 | −0.8 | 3481 | 708.9 | 708.9 | 708.9 | 10.66 |
| 939 | R.AEDGHEEAMETEASTSGEVAEVAEEAMETESSEKVGK.E | | 971.669 | 4 | 3883.654 | 3883.649 | 0 | 1.2 | 3481 | 642.2 | 642.2 | 642.2 | 9.85 |
| 948 | R.AEDGHEEAMETEASTSGEVAEVAEEAMETESSEK.V | | 1200.494 | 3 | 3599.466 | 3599.464 | 0 | 0.4 | 3481 | 593.4 | 593.4 | 593.4 | 9.11 |
| 939 | R.AEDGHEEAMETEASTSGEVAEVAEEAMETESSEKVGK.E | | 1295.223 | 3 | 3883.654 | 3883.649 | 0 | 1.1 | 3481 | 555.4 | 555.4 | 555.4 | 8.18 |
| 954 | R.AEDGHEEAMETEASTSGEVAEVAEEAMETESSEKVGKETSEL | | 1411.879 | 4 | 5644.493 | 5644.492 | 0 | 0.2 | 3481 | 611.6 | 611.6 | 611.6 | 8.10 |
| | GGSDVSILDTTR.L | | | | | | | | | | | | |
| 954 | R.AEDGHEEAMETEASTSGEVAEVAEEAMETESSEKVGKETSEL | | 1129.705 | 5 | 5644.495 | 5644.492 | 0 | 0.5 | 3481 | 629.5 | 629.5 | 629.5 | 7.88 |
| | GGSDVSILDTTR.L | | | | | | | | | | | | |
| 948 | R.AEDGHEEAMETEASTSGEVAEVAEEAMETESSEK.V | | 1200.495 | 3 | 3599.469 | 3599.464 | 0 | 1.3 | 3481 | 520.3 | 520.3 | 520.3 | 7.67 |
| 939 | R.AEDGHEEAMETEASTSGEVAEVAEEAMETESSEKVGK.E | | 777.536 | 5 | 3883.650 | 3883.649 | 0 | 0.1 | 3481 | 574.0 | 574.0 | 574.0 | 7.55 |
| 954 | R.AEDGHEEAMETEASTSGEVAEVAEEAMETESSEKVGKETSEL | | 1129.704 | 5 | 5644.491 | 5644.492 | 0 | −0.1 | 3481 | 556.3 | 556.3 | 556.3 | 6.98 |
| | GGSDVSILDTTR.L | | | | | | | | | | | | |
| 948 | R.AEDGHEEAMETEASTSGEVAEVAEEAMETESSEK.V | | 900.622 | 4 | 3599.467 | 3599.464 | 0 | 0.7 | 3481 | 452.9 | 452.9 | 452.9 | 6.59 |
| 954 | R.AEDGHEEAMETEASTSGEVAEVAEEAMETESSEKVGKETSEL | | 941.588 | 6 | 5644.491 | 5644.492 | 0 | −0.2 | 3481 | 592.6 | 592.6 | 592.6 | 6.14 |
| | GGSDVSILDTTR.L | | | | | | | | | | | | |
| 954 | R.AEDGHEEAMETEASTSGEVAEVAEEAMETESSEKVGKETSEL | | 1411.878 | 4 | 5644.489 | 5644.492 | 0 | −0.5 | 3481 | 542.1 | 542.1 | 542.1 | 6.06 |
| | GGSDVSILDTTR.L | | | | | | | | | | | | |
| 3073 | R.AEDGHEEAMETEASTSGEVAEVAEEAMETES[+79.966]SEK | S[+80] | 1145.900 | 5 | 5725.468 | 5724.458 | 1 | 1.2 | 3481 | 454.1 | 454.1 | 0.2 | 5.67 |
| | VGKETSELGGSDVSILDTTR.L | | | | | | | | | | | | |
| 4655 | R.AEDGHEEAMETEASTSGEVAEVAEEAMET[+79.966]ESSEK | T[+80] | 1146.099 | 5 | 5726.466 | 5724.458 | 2 | 0.2 | 3481 | 410.7 | 410.7 | 21.2 | 5.51 |
| | VGKETSELGGSDVSILDTTR.L | | | | | | | | | | | | |
| 3072 | R.AEDGHEEAMETEAS[+79.966]TSGEVAEVAEEAMETESSEK | S[+80] | 1145.700 | 5 | 5724.473 | 5724.458 | 0 | 2.5 | 3481 | 436.7 | 436.7 | 13.3 | 4.03 |
| | VGKETSELGGSDVSILDTTR.L | | | | | | | | | | | | |
| 954 | R.AEDGHEEAMETEASTSGEVAEVAEEAMETESSEKVGKETSEL | | 1412.380 | 4 | 5646.497 | 5644.492 | 2 | −0.3 | 3481 | 350.0 | 350.0 | 350.0 | 3.92 |
| | GGSDVSILDTTR.L | | | | | | | | | | | | |
| 988 | K.VGKETSELGGSDVSILDTTR.L | | 1032.526 | 2 | 2064.044 | 2064.046 | 0 | −0.7 | 3515 | 646.1 | 646.1 | 646.1 | 9.61 |
| 988 | K.VGKETSELGGSDVSILDTTR.L | | 688.687 | 3 | 2064.045 | 2064.046 | 0 | −0.2 | 3515 | 543.5 | 543.5 | 543.5 | 8.86 |
| 1119 | K.VGKETSELGGSDVSILDTTRLLR.S | | 612.335 | 4 | 2446.316 | 2446.315 | 0 | 0.6 | 3515 | 463.4 | 463.4 | 463.4 | 8.18 |
| 1827 | K.ETSELGGSDVSILDTTR.L | | 890.433 | 2 | 1779.860 | 1779.861 | 0 | −0.6 | 3518 | 687.7 | 687.7 | 687.7 | 10.87 |
| 827 | K.ETSELGGSDVSILDTTR.L | | 890.434 | 2 | 1779.861 | 1779.861 | 0 | 0.1 | 3518 | 459.2 | 459.2 | 459.2 | 8.78 |
| 827 | K.ETSELGGSDVSILDTTR.L | | 593.958 | 3 | 1779.860 | 1779.861 | 0 | −0.3 | 3518 | 314.5 | 314.5 | 314.5 | 5.25 |
| 1679 | R.SC[+57.021]VQSAVGMLRDQNESC[+57.021]TR.N | C[+57]* | 733.330 | 3 | 2197.976 | 2197.975 | 0 | 0.5 | 3538 | 535.9 | 535.9 | 535.9 | 8.86 |
| | | 2 | | | | | | | | | | | |
| 1713 | R.SC[+57.021]VQSAVGMLR.D | C[+57 | 604.300 | 2 | 1207.593 | 1207.592 | 0 | 0.4 | 3538 | 559.5 | 471.4 | 471.4 | 8.36 |
| 3279 | R.SC[+57.021]VQSAVGM[+15.995]LR.D | C[+57], | 612.298 | 2 | 1223.588 | 1223.587 | 0 | 0.6 | 3538 | 486.8 | 486.8 | 486.8 | 8.01 |
| | | M[+16] | | | | | | | | | | | |
| 3279 | R.SC[+57.021]VQSAVGM[+15.995]LR.D | C[+57], | 612.298 | 2 | 1223.588 | 1223.587 | 0 | 0.6 | 3538 | 355.1 | 355.1 | 355.1 | 6.04 |
| | | M[+16] | | | | | | | | | | | |
| 1727 | R.VVLLLGLLNEDDAC[+57.021]HASFLR.V | C[+57] | 752.400 | 3 | 2255.186 | 2255.185 | 0 | 0.4 | 3561 | 692.2 | 692.2 | 692.2 | 11.79 |
| 1727 | R.VVLLLGLLNEDDAC[+57.021]HASFLR.V | C[+57] | 752.400 | 3 | 2255.187 | 2255.185 | 0 | 0.6 | 3561 | 308.0 | 308.0 | 308.0 | 5.97 |
| 828 | R.LSVFLKKQEESQFHPLEWLAR.E | | 647.102 | 1 | 2585.388 | 2585.388 | 0 | 0.2 | 3586 | 484.6 | 484.6 | 484.6 | 7.84 |
| 828 | R.LSVFLKKQEESQFHPLEWLAR.E | | 518.084 | 5 | 2586.390 | 2585.388 | 1 | −0.2 | 3586 | 423.6 | 423.6 | 423.6 | 7.25 |
| 828 | R.LSVFLKKQEESQFHPLEWLAR.E | | 517.883 | 5 | 2585.386 | 2585.388 | 0 | −0.8 | 3586 | 405.1 | 405.1 | 405.1 | 6.55 |
| 1038 | K.KQEESQFHPLEWLAR.E | | 475.244 | 4 | 1897.956 | 1897.956 | 0 | 0.1 | 3592 | 427.8 | 427.8 | 427.8 | 7.64 |
| 1038 | K.KQEESQFHPLEWLAR.E | | 949.482 | 2 | 1897.956 | 1897.956 | 0 | 0.2 | 3592 | 533.3 | 533.3 | 533.3 | 7.57 |
| 1038 | K.KQEESQFHPLEWLAR.E | | 633.324 | 3 | 1897.958 | 1897.956 | 0 | 1.1 | 3592 | 378.5 | 378.5 | 378.5 | 7.53 |
| 859 | K.QEESQFHPLEWLAR.E | | 885.435 | 2 | 1769.863 | 1769.861 | 0 | 1.2 | 3593 | 697.1 | 697.1 | 697.1 | 11.58 |
| 859 | K.QEESQFHPLEWLAR.E | | 590.625 | 3 | 1769.861 | 1769.861 | 0 | 0.2 | 3593 | 462.0 | 462.0 | 462.0 | 8.02 |
| 1739 | R.EAC[+57.021]NQDALQEAGTFR.H | C[+57] | 855.381 | 2 | 1709.755 | 1709.755 | 0 | 0.3 | 3607 | 597.4 | 597.4 | 597.4 | 9.58 |
| 1739 | R.EAC[+57.021]NQDALQEAGTFR.H | C[+57] | 570.590 | 3 | 1709.755 | 1709.755 | 0 | 0.2 | 3607 | 482.9 | 482.9 | 482.9 | 7.47 |
| 1739 | R.EAC[+57.021]NQDALQEAGTFR.H | C[+57] | 570.590 | 3 | 1709.754 | 1709.755 | 0 | −0.3 | 3607 | 454.6 | 454.6 | 454.6 | 7.26 |
| 1739 | R.EAC[+57.021]NQDALQEAGTFR.H | C[+57] | 570.590 | 3 | 1709.755 | 1709.755 | 0 | −0.1 | 3607 | 405.0 | 405.0 | 405.0 | 6.90 |
| 1739 | R.EAC[+57.021]NQDALQEAGTFR.H | C[+57] | 570.590 | 3 | 1709.755 | 1709.755 | 0 | 0.1 | 3607 | 334.5 | 334.5 | 334.5 | 5.32 |
| 2234 | R.VQGAVTPLLASMISFIDR.D | | 640.021 | 3 | 1918.047 | 1918.047 | 0 | 0.4 | 3628 | 444.2 | 444.2 | 444.2 | 7.69 |
| 4035 | R.VQGAVTPLLASMISFIDRDGNLELLTRPDTPPWARDLWMFIFSD | | 1108.072 | 6 | 6643.398 | 6643.401 | 0 | −0.4 | 3628 | 497.1 | 497.1 | 497.1 | 4.23 |
| | TMLLNIPLVMNNER.H | | | | | | | | | | | | |
| 1214 | K.IKDYLEELWVQAQYITDAEGLPK.K | | 908.138 | 3 | 2722.400 | 2722.397 | 0 | 0.8 | 3715 | 613.0 | 613.0 | 613.0 | 10.53 |
| 1094 | K.IKDYLEELWVQAQYITDAEGLPKK.F | | 713.379 | 4 | 2850.494 | 2850.492 | 0 | 0.6 | 3715 | 502.3 | 502.3 | 502.3 | 8.39 |
| 1094 | K.IKDYLEELWVQAQYITDAEGLPKK.F | | 570.905 | 5 | 2850.494 | 2850.492 | 0 | 0.4 | 3715 | 556.5 | 556.5 | 556.5 | 7.98 |
| 1214 | K.IKDYLEELWVQAQYITDAEGLPK.K | | 681.356 | 4 | 2722.400 | 2722.397 | 0 | 1.0 | 3715 | 311.8 | 311.8 | 311.8 | 4.99 |
| 1856 | K.KFVDIFQQTPLGR.F | | 774.930 | 2 | 1548.853 | 1548.853 | 0 | −0.4 | 3738 | 447.4 | 447.4 | 447.4 | 8.02 |
| 1379 | R.KLKAASEAPEEEVSLPWVHLAYQR.F | | 688.617 | 4 | 2751.447 | 2751.446 | 0 | 0.1 | 3797 | 675.3 | 675.3 | 675.3 | 11.53 |
| 1379 | R.KLKAASEAPEEEVSLPWVHLAYQR.F | | 551.095 | 5 | 2751.447 | 2751.446 | 0 | 0.3 | 3797 | 375.4 | 375.4 | 375.4 | 7.27 |
| 1379 | R.KLKAASEAPEEEVSLPWVHLAYQR.F | | 688.618 | 4 | 2751.449 | 2751.446 | 0 | 0.8 | 3797 | 335.0 | 335.0 | 335.0 | 5.99 |
| 1167 | K.LKAASEAPEEEVSLPWVHLAYQR.F | | 656.593 | 4 | 2623.351 | 2623.352 | 0 | −0.3 | 3798 | 679.8 | 679.8 | 679.8 | 11.53 |
| 1167 | K.LKAASEAPEEEVSLPWVHLAYQR.F | | 875.122 | 3 | 2623.351 | 2623.352 | 0 | −0.2 | 3798 | 668.2 | 668.2 | 668.2 | 11.53 |
| 1273 | K.AASEAPEEEVSLPWVHLAYQR.F | | 794.729 | 3 | 2382.173 | 2382.173 | 0 | 0.2 | 3800 | 682.3 | 682.3 | 682.3 | 11.79 |
| 1273 | K.AASEAPEEEVSLPWVHLAYQR.F | | 1191.590 | 2 | 2382.172 | 2382.173 | 0 | −0.3 | 3800 | 598.7 | 598.7 | 598.7 | 9.79 |
| 947 | R.ILTIYPQVLHSLMEAR.W | | 628.686 | 3 | 1884.043 | 1884.041 | 0 | 0.7 | 3831 | 597.9 | 597.9 | 597.9 | 9.58 |
| 947 | R.ILTIYPQVLHSLMEAR.W | | 628.685 | 3 | 1884.041 | 1884.041 | 0 | 0.1 | 3831 | 521.8 | 521.8 | 521.8 | 8.91 |
| 947 | R.ILTIYPQVLHSLMEAR.W | | 628.685 | 3 | 1884.042 | 1884.041 | 0 | 0.2 | 3831 | 475.7 | 475.7 | 475.7 | 8.57 |
| 947 | R.ILTIYPQVLHSLMEAR.W | | 942.525 | 2 | 1884.043 | 1884.041 | 0 | 1.0 | 3831 | 475.3 | 475.3 | 475.3 | 8.57 |
| 947 | R.ILTIYPQVLHSLMEAR.W | | 628.686 | 3 | 1884.042 | 1884.041 | 0 | 0.3 | 3831 | 453.4 | 453.4 | 453.4 | 8.02 |
| 1588 | R.ILTIYPQVLHSLM[+15.995]EAR.W | M[+16] | 951.023 | 2 | 1901.039 | 1900.036 | 1 | −0.1 | 3831 | 366.8 | 366.8 | 366.8 | 7.53 |
| 1588 | R.ILTIYPQVLHSLM[+15.995]EAR.W | M[+16] | 634.351 | 3 | 1901.039 | 1900.036 | 1 | 0.0 | 3831 | 354.3 | 354.3 | 354.3 | 6.24 |
| 1588 | R.ILTIYPQVLHSLM[+15.995]EAR.W | M[+16] | 634.017 | 3 | 1900.036 | 1900.036 | 0 | −0.1 | 3831 | 308.3 | 308.3 | 308.3 | 5.97 |
| 992 | R.NTLKPSPQAWLQLVK.N | | 575.001 | 3 | 1722.990 | 1722.990 | 0 | −0.3 | 3873 | 720.3 | 720.3 | 720.3 | 12.58 |
| 992 | R.NTLKPSPQAWLQLVK.N | | 575.002 | 3 | 1722.990 | 1722.990 | 0 | 0.1 | 3873 | 676.3 | 676.3 | 676.3 | 11.58 |
| 992 | R.NTLKPSPQAWLQLVK.N | | 575.002 | 3 | 1722.990 | 1722.990 | 0 | −0.1 | 3873 | 659.9 | 659.9 | 659.9 | 11.58 |
| 992 | R.NTLKPSPQAWLQLVK.N | | 861.999 | 2 | 1722.990 | 1722.990 | 0 | 0.1 | 3873 | 559.8 | 559.8 | 559.8 | 8.91 |
| 2814 | R.IFSTALFVEHVLLGTESR.V | | 673.702 | 3 | 2019.093 | 2019.091 | 0 | 0.8 | 3923 | 662.9 | 662.9 | 662.9 | 11.79 |
| 2814 | R.IFSTALFVEHVLLGTESR.V | | 673.703 | 3 | 2019.095 | 2019.091 | 0 | 2.0 | 3923 | 521.9 | 521.9 | 521.9 | 7.27 |
| 1047 | R.VPELQGLVTEHVFLLDK.C | | 646.363 | 3 | 1937.075 | 1937.074 | 0 | 0.2 | 3941 | 575.0 | 575.0 | 575.0 | 9.79 |
| 1047 | R.VPELQGLVTEHVFLLDK.C | | 969.043 | 2 | 1937.078 | 1937.074 | 0 | 2.1 | 3941 | 422.3 | 422.3 | 422.3 | 7.32 |
| 3298 | K.C[+57.021]LRENSDVKTHGPFEAVMR.T | C[+57] | 562.272 | 4 | 2246.067 | 2246.081 | 0 | −5.9 | 3958 | 333.1 | 333.1 | 333.1 | 4.02 |
| 1090 | R.ENSDVKTHGPFEAVMR.T | | 606.293 | 3 | 1816.865 | 1816.865 | 0 | 0.2 | 3961 | 645.6 | 645.6 | 645.6 | 10.32 |
| 1090 | R.ENSDVKTHGPFEAVMR.T | | 606.293 | 3 | 1816.864 | 1816.865 | 0 | −0.6 | 3961 | 626.3 | 626.3 | 626.3 | 9.41 |
| 1090 | R.ENSDVKTHGPFEAVMR.T | | 454.972 | 4 | 1816.866 | 1816.865 | 0 | 0.5 | 3961 | 443.6 | 443.6 | 443.6 | 8.31 |
| 1090 | R.ENSDVKTHGPFEAVMR.T | | 908.936 | 2 | 1816.864 | 1816.865 | 0 | −0.1 | 3961 | 450.5 | 450.5 | 450.5 | 6.42 |
| 1090 | R.ENSDVKTHGPFEAVMR.T | | 454.972 | 4 | 1816.864 | 1816.865 | 0 | −0.2 | 3961 | 358.3 | 358.3 | 358.3 | 5.78 |
| 825 | K.THGPFEAVMR.T | | 572.782 | 2 | 1144.557 | 1144.557 | 0 | 0.0 | 3967 | 477.2 | 477.2 | 477.2 | 7.80 |
| 825 | K.THGPFEAVMR.T | | 572.782 | 2 | 1144.556 | 1144.557 | 0 | −0.3 | 3967 | 467.6 | 467.6 | 467.6 | 7.80 |
| 825 | K.THGPFEAVMR.T | | 572.782 | 2 | 1144.557 | 1144.557 | 0 | 0.0 | 3967 | 421.6 | 421.6 | 421.6 | 7.44 |
| 1681 | R.AWFASEQMIC[+57.021]PYC[+57.021]LTALPDEFSPAVS | C[+57]* | 1161.538 | 3 | 3482.600 | 3482.597 | 0 | 0.8 | 4023 | 791.4 | 693.2 | 693.2 | 13.12 |
| | QAHR.E | 2 | | | | | | | | | | | |
| 856 | K.DNAPPEKEVIESLLSLLFVQK.G | | 790.438 | 3 | 2369.298 | 2369.296 | 0 | 0.7 | 4080 | 563.9 | 563.9 | 563.9 | 9.53 |
| 856 | K.DNAPPEKEVIESLLSLLFVQK.G | | 1185.153 | 2 | 2369.298 | 2369.296 | 0 | 0.7 | 4080 | 444.4 | 444.4 | 444.4 | 7.97 |
| 856 | K.DNAPPEKEVIESLLSLLFVQK.G | | 593.080 | 4 | 2369.297 | 2369.296 | 0 | 0.4 | 4080 | 366.4 | 366.4 | 366.4 | 6.94 |
| 2072 | K.SLSPFNDVVDKTPVIR.S | | 596.328 | 3 | 1786.970 | 1786.970 | 0 | 0.2 | 4116 | 488.0 | 488.0 | 488.0 | 8.52 |
| 2072 | K.SLSPFNDVVDKTPVIR.S | | 893.989 | 2 | 1786.971 | 1786.970 | 0 | 0.5 | 4116 | 466.0 | 466.0 | 466.0 | 8.31 |
| 2072 | K.SLSPFNDVVDKTPVIR.S | | 596.328 | 3 | 1786.970 | 1786.970 | 0 | −0.2 | 4116 | 459.2 | 459.2 | 459.2 | 7.54 |
| 1408 | K.TSAYSRNDELNHLEEEGR.F | | 707.327 | 3 | 2119.965 | 2119.964 | 0 | 0.5 | 4186 | 731.0 | 731.0 | 731.0 | 12.53 |
| 936 | K.TSAYSRNDELNHLEEEGRFLK.A | | 627.808 | 4 | 2508.212 | 2508.211 | 0 | 0.1 | 4186 | 430.7 | 430.7 | 430.7 | 7.47 |
| 1408 | K.TSAYSRNDELNHLEEEGR.F | | 530.746 | 4 | 2119.963 | 2119.964 | 0 | −0.4 | 4186 | 382.6 | 382.6 | 382.6 | 7.47 |
| 936 | K.TSAYSRNDELNHLEEEGRFLK.A | | 502.649 | 5 | 2509.215 | 2508.211 | 1 | 0.2 | 4186 | 424.5 | 424.5 | 21.6 | 7.25 |
| 936 | K.TSAYSRNDELNHLEEEGRFLK.A | | 502.448 | 5 | 2508.210 | 2508.211 | 0 | −0.4 | 4186 | 371.6 | 371.6 | 371.6 | 7.14 |
| 936 | K.TSAYSRNDELNHLEEEGRFLK.A | | 836.741 | 3 | 2508.209 | 2508.211 | 0 | −0.8 | 4186 | 598.5 | 598.5 | 598.5 | 6.93 |
| 936 | K.TSAYSRNDELNHLEEEGRFLK.A | | 836.742 | 3 | 2508.210 | 2508.211 | 0 | −0.4 | 4186 | 535.9 | 535.9 | 535.9 | 6.26 |
| 936 | K.TSAYSRNDELNHLEEEGRFLK.A | | 502.448 | 5 | 2508.210 | 2508.211 | 0 | −0.6 | 4186 | 372.7 | 372.7 | 372.7 | 6.22 |
| 936 | K.TSAYSRNDELNHLEEEGRFLK.A | | 627.809 | 4 | 2508.212 | 2508.211 | 0 | 0.3 | 4186 | 317.1 | 317.1 | 317.1 | 5.37 |
| 1408 | K.TSAYSRNDELNHLEEEGR.F | | 1060.485 | 2 | 2119.962 | 2119.964 | 0 | −0.8 | 4186 | 311.0 | 311.0 | 311.0 | 3.65 |
| 3090 | K.TSAYSRNDELN[+0.984]HLEEEGR.F | N[+1] | 1061.489 | 2 | 2121.970 | 2120.948 | 1 | 9.0 | 4186 | 330.3 | 330.3 | 23.8 | 3.27 |
| 1494 | R.N[+0.984]DELNHLEEEGRFLK.A | N[+1] | 615.299 | 3 | 1843.882 | 1843.882 | 0 | −0.3 | 4192 | 703.3 | 703.3 | 312.3 | 12.32 |
| 978 | R.NDELNHLEEEGRFLK.A | | 614.971 | 3 | 1842.898 | 1842.898 | 0 | −0.1 | 4192 | 629.6 | 629.6 | 629.6 | 10.32 |
| 807 | R.NDELNHLEEEGR.F | | 485.555 | 3 | 1454.650 | 1454.651 | 0 | −0.3 | 4192 | 585.3 | 585.3 | 585.3 | 9.58 |
| 978 | R.NDELNHLEEEGRFLK.A | | 614.971 | 3 | 1842.898 | 1842.898 | 0 | −0.2 | 4192 | 599.8 | 599.8 | 599.8 | 9.32 |
| 1496 | R.NDELN[+0.984]HLEEEGR.F | N[+1] | 485.883 | 3 | 1455.634 | 1455.635 | 0 | −0.1 | 4192 | 493.7 | 493.7 | 310.7 | 8.23 |
| 807 | R.NDELNHLEEEGR.F | | 485.555 | 3 | 1454.650 | 1454.651 | 0 | −0.2 | 4192 | 482.8 | 482.8 | 482.8 | 8.01 |
| 978 | R.NDELNHLEEEGRFLK.A | | 461.480 | 4 | 1842.898 | 1842.898 | 0 | 0.0 | 4192 | 486.6 | 486.6 | 486.6 | 7.97 |
| 807 | R.NDELNHLEEEGR.F | | 727.828 | 2 | 1454.650 | 1454.651 | 0 | −0.8 | 4192 | 498.0 | 498.0 | 498.0 | 7.86 |
| 1488 | R.NDELN[+0.984]HLEEEGRFLK.A | N[+1] | 615.299 | 3 | 1843.882 | 1843.882 | 0 | −0.3 | 4192 | 420.0 | 420.0 | 336.6 | 7.60 |
| 1488 | R.NDELN[+0.984]HLEEEGRFLK.A | N[+1] | 461.726 | 4 | 1843.882 | 1843.882 | 0 | −0.1 | 4192 | 418.7 | 418.7 | 37.0 | 7.60 |
| 1494 | R.N[+0.984]DELNHLEEEGRFLK.A | N[+1] | 461.726 | 4 | 1843.882 | 1843.882 | 0 | −0.1 | 4192 | 386.9 | 386.9 | 85.6 | 7.27 |
| 1494 | R.N[+0.984]DELNHLEEEGRFLK.A | N[+1] | 461.726 | 4 | 1843.882 | 1843.882 | 0 | 0.1 | 4192 | 374.3 | 374.3 | 54.1 | 7.27 |
| 978 | R.NDELNHLEEEGRFLK.A | | 461.480 | 4 | 1842.899 | 1842.898 | 0 | 0.3 | 4192 | 370.0 | 370.0 | 370.0 | 7.06 |
| 978 | R.NDELNHLEEEGRFLK.A | | 461.480 | 1 | 1842.899 | 1842.898 | 0 | 0.3 | 4192 | 358.6 | 358.6 | 358.6 | 5.78 |
| 1496 | R.NDELN[+0.984]HLEEEGR.F | N[+1] | 486.217 | 3 | 1456.637 | 1455.635 | 1 | −0.6 | 4192 | 466.8 | 466.8 | 349.0 | 5.10 |
| 978 | R.NDELNHLEEEGRFLK.A | | 461.733 | 4 | 1843.910 | 1842.898 | 1 | 4.6 | 4192 | 322.8 | 322.8 | 322.8 | 4.17 |
| 2409 | R.FLKAYSPASR.G | | 570.314 | 2 | 1139.621 | 1139.621 | 0 | 0.3 | 4204 | 455.3 | 455.3 | 455.3 | 7.54 |
| 831 | R.GREPANEASVEYLQEVAR.I | | 1009.501 | 2 | 2017.994 | 2017.994 | 0 | 0.2 | 4214 | 532.6 | 532.6 | 532.6 | 8.86 |
| 831 | R.GREPANEASVEYLQEVAR.I | | 1009.501 | 2 | 2017.994 | 2017.994 | 0 | 0.3 | 4214 | 469.1 | 469.1 | 469.1 | 8.52 |
| 831 | R.GREPANEASVEYLQEVAR.I | | 673.336 | 3 | 2017.994 | 2017.994 | 0 | 0.2 | 4214 | 511.1 | 511.1 | 511.1 | 8.18 |
| 831 | R.GREPANEASVEYLQEVAR.I | | 673.337 | 3 | 2017.995 | 2017.994 | 0 | 0.8 | 4214 | 455.3 | 455.3 | 455.3 | 7.97 |
| 831 | R.GREPANEASVEYLQEVAR.I | | 673.336 | 3 | 2017.994 | 2017.994 | 0 | 0.1 | 4214 | 412.4 | 412.4 | 412.4 | 7.39 |
| 831 | R.GREPANEASVEYLQEVAR.I | | 505.254 | 4 | 2017.995 | 2017.994 | 0 | 0.4 | 4214 | 339.8 | 339.8 | 339.8 | 5.45 |
| 1696 | R.IRLC[+57.021]LDR.A | C[+57] | 473.269 | 2 | 945.530 | 945.530 | 0 | −0.1 | 4232 | 362.8 | 362.8 | 362.8 | 6.48 |
| 929 | R.AADFLSEPEGGPEMAK.E | | 824.880 | 2 | 1648.753 | 1648.752 | 0 | 0.5 | 4239 | 655.4 | 655.4 | 655.4 | 11.58 |
| 1232 | R.AADFLSEPEGGPEMAKEK.Q | | 635.969 | 3 | 1905.891 | 1905.890 | 0 | 0.6 | 4239 | 647.5 | 647.5 | 647.5 | 10.53 |
| 1232 | R.AADFLSEPEGGPEMAKEK.Q | | 953.449 | 2 | 1905.890 | 1905.890 | 0 | 0.3 | 4239 | 587.0 | 587.0 | 587.0 | 9.53 |
| 1232 | R.AADFLSEPEGGPEMAKEK.Q | | 635.969 | 3 | 1905.891 | 1905.890 | 0 | 0.6 | 4239 | 566.6 | 566.6 | 566.6 | 9.53 |
| 1624 | R.AADFLSEPEGGPEM[+15.995]AKEK.Q | M[+16] | 641.300 | 3 | 1921.885 | 1921.885 | 0 | −0.1 | 4239 | 554.3 | 554.3 | 554.3 | 8.31 |
| 1232 | R.AADFLSEPEGGPEMAKEK.Q | | 477.228 | 4 | 1905.890 | 1905.890 | 0 | 0.3 | 4239 | 498.8 | 498.8 | 498.8 | 8.19 |
| 1618 | R.AADFLSEPEGGPEM[+15.995]AK.E | M[+16] | 832.877 | 2 | 1664.747 | 1664.747 | 0 | 0.1 | 4239 | 494.7 | 494.7 | 494.7 | 8.01 |
| 1624 | R.AADFLSEPEGGPEM[+15.995]AKEK.Q | M[+16] | 641.300 | 3 | 1921.886 | 1921.885 | 0 | 0.6 | 4239 | 484.4 | 484.4 | 484.4 | 7.95 |
| 929 | R.AADFLSEPEGGPEMAK.E | | 550.256 | 3 | 1648.753 | 1648.752 | 0 | 0.1 | 4239 | 443.5 | 443.5 | 443.5 | 7.26 |
| 1232 | R.AADFLSEPEGGPEMAKEK.Q | | 635.968 | 3 | 1905.891 | 1905.890 | 0 | 0.3 | 4239 | 331.8 | 331.8 | 331.8 | 5.99 |
| 1036 | R.GMEFVQGLSKPGRPHQWVFPK.D | | 809.091 | 3 | 2425.259 | 2425.260 | 0 | −0.4 | 4288 | 615.1 | 615.1 | 615.1 | 9.45 |
| 1036 | R.GMEFVQGLSKPGRPHQWVFPK.D | | 607.070 | 4 | 2425.260 | 2425.260 | 0 | 0.0 | 4288 | 372.9 | 372.9 | 372.9 | 7.96 |
| 1551 | R.GM[+15.995]EFVQGLSKPGRPHQWVFPK.D | M[+16] | 489.057 | 5 | 2441.255 | 2441.255 | 0 | 0.0 | 4288 | 367.7 | 367.7 | 367.7 | 7.96 |
| 1551 | R.GM[+15.995]EFVQGLSKPGRPHQWVFPK.D | M[+16] | 611.069 | 4 | 2441.256 | 2441.255 | 0 | 0.4 | 4288 | 371.3 | 365.4 | 365.4 | 7.73 |
| 3674 | R.GMEFVQGLSK.P | | 548.279 | 2 | 1095.550 | 1095.550 | 0 | −0.1 | 4288 | 397.9 | 397.9 | 397.9 | 7.32 |
| 1551 | R.GM[+15.995]EFVQGLSKPGRPHQWVFPK.D | M[+16] | 814.424 | 3 | 2441.257 | 2441.255 | 0 | 0.7 | 4288 | 512.5 | 512.5 | 512.5 | 7.09 |
| 1036 | R.GMEFVQGLSKPGRPHQWVFPK.D | | 809.091 | 3 | 2425.260 | 2425.260 | 0 | −0.1 | 4288 | 499.1 | 499.1 | 499.1 | 7.09 |
| 1036 | R.GMEFVQGLSKPGRPHQWVFPK.D | | 607.070 | 4 | 2425.260 | 2425.260 | 0 | 0.0 | 4288 | 356.9 | 356.9 | 356.9 | 6.67 |
| 1058 | K.DVVKQQGLRQDHPGQMDR.Y | | 703.020 | 3 | 2107.047 | 2107.046 | 0 | 0.3 | 4309 | 589.1 | 589.1 | 589.1 | 7.85 |
| 1058 | K.DVVKQQGLRQDHPGQMDR.Y | | 527.517 | 4 | 2107.047 | 2107.046 | 0 | 0.3 | 4309 | 348.1 | 348.1 | 348.1 | 5.85 |
| 1058 | K.DVVKQQGLRQDHPGQMDR.Y | | 422.215 | 5 | 2107.047 | 2107.046 | 0 | 0.5 | 4309 | 311.6 | 311.6 | 311.6 | 5.37 |
| 1544 | K.QQGLRQDHPGQM[+15.995]DRYLVYGDEYK.A | M[+16] | 703.833 | 4 | 2812.311 | 2812.311 | 0 | 0.0 | 4313 | 394.7 | 394.7 | 394.7 | 7.14 |
| 1880 | K.QQGLRQDHPGQMDRYLVYGDEYK.A | | 560.269 | 5 | 2797.317 | 2796.316 | 1 | −0.6 | 4313 | 368.5 | 368.5 | 368.5 | 6.22 |
| 1880 | K.QQGLRQDHPGQMDRYLVYGDEYK.A | | 560.069 | 5 | 2796.316 | 2796.316 | 0 | 0.1 | 4313 | 338.4 | 338.4 | 338.4 | 5.65 |
| 1880 | K.QQGLRQDHPGQMDRYLVYGDEYK.A | | 932.776 | 3 | 2796.315 | 2796.316 | 0 | −0.4 | 4313 | 439.5 | 439.5 | 439.5 | 5.21 |
| 1880 | K.QQGLRQDHPGQMDRYLVYGDEYK.A | | 699.834 | 4 | 2796.316 | 2796.316 | 0 | −0.1 | 4313 | 314.5 | 314.5 | 314.5 | 5.19 |
| 816 | R.QDHPGQMDRYLVYGDEYK.A | | 738.669 | 3 | 2213.992 | 2213.992 | 0 | 0.1 | 4318 | 449.6 | 449.6 | 449.6 | 7.97 |
| 816 | R.QDHPGQMDRYLVYGDEYK.A | | 554.254 | 4 | 2213.993 | 2213.992 | 0 | 0.4 | 4318 | 407.7 | 407.7 | 407.7 | 7.59 |
| 3802 | R.QDHPGQMDRYLVYGDEYKALR.D | | 511.649 | 5 | 2554.214 | 2554.214 | 0 | −0.2 | 4318 | 434.5 | 434.5 | 434.5 | 7.47 |
| 3802 | R.QDHPGQMDRYLVYGDEYKALR.D | | 511.649 | 5 | 2554.215 | 2554.214 | 0 | 0.4 | 4318 | 418.5 | 418.5 | 418.5 | 7.25 |
| 816 | R.QDHPGQMDRYLVYGDEYK.A | | 554.254 | 4 | 2213.992 | 2213.992 | 0 | 0.1 | 4318 | 371.0 | 371.0 | 371.0 | 7.09 |
| 970 | R.YLVYGDEYKALRDAVAK.A | | 494.264 | 4 | 1974.033 | 1974.033 | 0 | −0.1 | 4327 | 415.5 | 415.5 | 415.5 | 8.02 |
| 1785 | R.YLVYGDEYK.A | | 575.277 | 2 | 1149.546 | 1149.546 | 0 | −0.2 | 4327 | 453.2 | 453.2 | 453.2 | 7.80 |
| 844 | R.YLVYGDEYKALR.D | | 497.261 | 3 | 1489.769 | 1489.769 | 0 | 0.1 | 4327 | 488.7 | 488.7 | 488.7 | 7.75 |
| 1844 | R.YLVYGDEYKALR.D | | 497.261 | 3 | 1489.769 | 1489.769 | 0 | 0.3 | 4327 | 482.9 | 482.9 | 482.9 | 7.75 |
| 970 | R.YLVYGDEYKALRDAVAK.A | | 658.683 | 3 | 1974.035 | 1974.033 | 0 | 0.7 | 4327 | 455.2 | 455.2 | 455.2 | 7.63 |
| 1785 | R.YLVYGDEYK.A | | 575.277 | 2 | 1149.546 | 1149.546 | 0 | −0.3 | 4327 | 448.2 | 448.2 | 448.2 | 7.60 |
| 1785 | R.YLVYGDEYK.A | | 575.277 | 2 | 1149.546 | 1149.546 | 0 | 0.0 | 4327 | 439.9 | 439.9 | 439.9 | 7.44 |
| 1785 | R.YLVYGDEYK.A | | 575.277 | 2 | 1149.546 | 1149.546 | 0 | 0.0 | 4327 | 429.5 | 429.5 | 429.5 | 7.44 |
| 844 | R.YLVYGDEYKALR.D | | 497.261 | 3 | 1489.769 | 1489.769 | 0 | 0.1 | 4327 | 434.7 | 434.7 | 434.7 | 7.38 |
| 970 | R.YLVYGDEYKALRDAVAK.A | | 494.264 | 4 | 1974.034 | 1974.033 | 0 | 0.3 | 4327 | 378.0 | 378.0 | 378.0 | 7.14 |
| 970 | R.YLVYGDEYKALRDAVAK.A | | 658.683 | 3 | 1974.034 | 1974.033 | 0 | 0.5 | 4327 | 363.3 | 363.3 | 363.3 | 7.14 |
| 1785 | R.YLVYGDEYK.A | | 575.277 | 2 | 1149.547 | 1149.546 | 0 | 0.2 | 4327 | 386.2 | 386.2 | 386.2 | 6.87 |
| 1970 | R.YLVYGDEYKALRDAVAK.A | | 987.520 | 2 | 1974.034 | 1974.033 | 0 | 0.2 | 4327 | 469.0 | 469.0 | 469.0 | 6.84 |
| 970 | R.YLVYGDEYKALRDAVAK.A | | 494.264 | 4 | 1974.034 | 1974.033 | 0 | 0.6 | 4327 | 329.2 | 329.2 | 329.2 | 5.85 |
| 1726 | K.AVLEC[+57.021]KPLGIK.T | C[+57] | 409.909 | 3 | 1227.713 | 1227.713 | 0 | −0.1 | 4344 | 511.3 | 511.3 | 511.3 | 8.23 |
| 1726 | K.AVLEC[+57.021]KPLGIK.T | C[+57] | 409.909 | 3 | 1227.713 | 1227.713 | 0 | 0.1 | 4344 | 351.0 | 351.0 | 351.0 | 6.04 |
| 3314 | K.AC[+57.021]KTPQSQQSAYFLLTLFR.E | C[+57] | 753.725 | 3 | 2259.160 | 2259.159 | 0 | 0.5 | 4359 | 301.9 | 301.9 | 301.9 | 5.53 |
| 1194 | K.TPQSQQSAYFLLTLFR.E | | 634.004 | 3 | 1899.998 | 1899.996 | 0 | 0.9 | 4362 | 376.3 | 376.3 | 376.3 | 7.21 |
| 1878 | R.EVAILYR.S | | 432.253 | 2 | 863.499 | 863.499 | 0 | 0.1 | 4378 | 418.1 | 418.1 | 418.1 | 7.44 |
| 1878 | R.EVAILYR.S | | 432.253 | 2 | 863.499 | 863.499 | 0 | −0.1 | 4378 | 371.6 | 371.6 | 371.6 | 7.32 |
| 1878 | R.EVAILYR.S | | 432.253 | 2 | 863.499 | 863.499 | 0 | −0.1 | 4378 | 301.3 | 301.3 | 301.3 | 5.58 |
| 1685 | R.SHNASLHPTPEQC[+57.021]EAVSK.F | C[+57] | 664.647 | 3 | 1991.925 | 1991.924 | 0 | 0.6 | 4385 | 653.3 | 653.3 | 653.3 | 11.79 |
| 1685 | R.SHNASLHPTPEQC[+57.021]EAVSK.F | C[+57] | 664.647 | 3 | 1991.925 | 1991.924 | 0 | 0.5 | 4385 | 636.0 | 636.0 | 636.0 | 10.79 |
| 1685 | R.SHNASLHPTPEQC[+57.021]EAVSK.F | C[+57] | 498.736 | 4 | 1991.924 | 1991.924 | 0 | −0.1 | 4385 | 443.9 | 443.9 | 443.9 | 8.23 |
| 1759 | K.ILSPPDISR.F | | 499.287 | 2 | 997.568 | 997.568 | 0 | −0.1 | 4409 | 389.3 | 389.3 | 389.3 | 7.14 |
| 1759 | K.ILSPPDISR.F | | 499.287 | 2 | 997.568 | 997.568 | 0 | −0.2 | 4409 | 335.8 | 335.8 | 335.8 | 6.04 |
| 1759 | K.ILSPPDISR.F | | 499.287 | 2 | 997.567 | 997.568 | 0 | −0.3 | 4409 | 355.1 | 355.1 | 355.1 | 5.86 |
| 1759 | K.ILSPPDISR.F | | 499.288 | 2 | 997.568 | 997.568 | 0 | 0.1 | 4409 | 338.5 | 338.5 | 338.5 | 5.86 |
| 1759 | K.ILSPPDISR.F | | 499.288 | 2 | 997.568 | 997.568 | 0 | 0.2 | 4409 | 315.4 | 315.4 | 315.4 | 5.76 |
| 1759 | K.ILSPPDISR.F | | 499.287 | 2 | 997.567 | 997.568 | 0 | −0.2 | 4409 | 300.9 | 300.9 | 300.9 | 5.31 |
| 1782 | K.ILSPPDISRFATSLVDNSVPLLR.A | | 628.356 | 4 | 2510.400 | 2510.398 | 0 | 1.1 | 4409 | 304.2 | 304.2 | 304.2 | 5.17 |
| 3269 | K.ILSPPDIS[+79.966]RFAT[+79.966]SLVDNSVPLLRAGP | C[+57], | 1062.016 | 6 | 6367.058 | 6367.072 | 0 | −2.1 | 4409 | 359.2 | 359.2 | 3.9 | 2.07 |
| | SDSN[+0.984]LDGTVT[+79.966]EMAIHAAAVLLC | N[+1], | | | | | | | | | | | |
| | [+57.021]GQNELLEPLK.N, | S[+80] | | | | | | | | | | | |
| | | T[+80]* | | | | | | | | | | | |
| | | 2 | | | | | | | | | | | |
| 887 | R.FATSLVDNSVPLLR.A | | 766.428 | 2 | 1531.848 | 1531.848 | 0 | −0.1 | 4418 | 620.6 | 620.6 | 620.6 | 10.58 |
| 887 | R.FATSLVDNSVPLLR.A | | 766.427 | 2 | 1531.848 | 1531.848 | 0 | −0.2 | 4418 | 590.5 | 590.5 | 590.5 | 9.58 |
| 887 | R.FATSLVDNSVPLLR.A | | 766.427 | 2 | 1531.848 | 1531.848 | 0 | −0.2 | 4418 | 556.6 | 556.6 | 556.6 | 8.91 |
| 887 | R.FATSLVDNSVPLLR.A | | 766.428 | 2 | 1531.848 | 1531.848 | 0 | −0.1 | 4418 | 545.2 | 500.5 | 500.5 | 8.91 |
| 1485 | R.FATSLVDN[+0.984]SVPLLR.A | N[+1] | 766.920 | 2 | 1532.833 | 1532.832 | 0 | 0.6 | 4418 | 489.2 | 489.2 | 489.2 | 8.78 |
| 1485 | R.FATSLVDN[+0.984]SVPLLR.A | N[+1] | 766.919 | 2 | 1532.831 | 1532.832 | 0 | −0.4 | 4418 | 582.1 | 582.1 | 582.1 | 8.67 |
| 887 | R.FATSLVDNSVPLLR.A | | 511.287 | 3 | 1531.848 | 1531.848 | 0 | −0.2 | 4418 | 450.1 | 450.1 | 450.1 | 8.03 |
| 887 | R.FATSLVDNSVPLLR.A | | 766.427 | 2 | 1531.848 | 1531.848 | 0 | −0.2 | 4418 | 420.5 | 420.5 | 420.5 | 7.86 |
| 1485 | R.FATSLVDN[+0.984]SVPLLR.A | N[+1] | 511.615 | 3 | 1532.831 | 1532.832 | 0 | −0.3 | 4418 | 425.6 | 425.6 | 425.6 | 7.32 |
| 887 | R.FATSLVDNSVPLLR.A | | 511.287 | 3 | 1531.848 | 1531.848 | 0 | −0.1 | 4418 | 420.0 | 420.0 | 420.0 | 7.10 |
| 887 | R.FATSLVDNSVPLLR.A | | 511.288 | 3 | 1531.848 | 1531.848 | 0 | 0.4 | 4418 | 373.4 | 373.4 | 373.4 | 6.79 |
| 1098 | K.NLAFSPATMAHAFLPTMPEDLLAQAR.R | | 938.477 | 3 | 2813.416 | 2813.411 | 0 | 1.8 | 4467 | 799.6 | 799.6 | 799.6 | 12.49 |
| 1628 | K.NLAFSPATMAHAFLPTM[+15.995]PEDLLAQAR.R | M[+16] | 943.807 | 3 | 2829.406 | 2829.406 | 0 | −0.3 | 4467 | 674.3 | 674.3 | 160.7 | 11.79 |
| 1098 | K.NLAFSPATMAHAFLPTMPEDLLAQAR.R | | 1407.212 | 2 | 2813.417 | 2813.411 | 0 | 2.1 | 4467 | 712.1 | 600.9 | 600.9 | 11.49 |
| 1639 | K.NLAFSPATM[+15.995]AHAFLPTMPEDLLAQAR.R | M[+16] | 943.808 | 3 | 2829.408 | 2829.406 | 0 | 0.7 | 4467 | 662.2 | 662.2 | 68.0 | 11.24 |
| 1639 | K.NLAFSPATM[+15.995]AHAFLPTMPEDLLAQAR.R | M[+16] | 943.807 | 3 | 2829.407 | 2829.406 | 0 | 0.2 | 4467 | 632.7 | 632.7 | 71.1 | 10.79 |
| 941 | K.NLAFSPATMAHAFLPTMPEDLLAQARR.W | | 743.135 | 4 | 2969.517 | 2969.512 | 0 | 1.4 | 4467 | 569.8 | 569.8 | 569.8 | 9.53 |
| 1098 | K.NLAFSPATMAHAFLPTMPEDLLAQAR.R | | 704.110 | 4 | 2813.416 | 2813.411 | 0 | 1.8 | 4467 | 601.1 | 566.9 | 566.9 | 8.95 |
| 1628 | K.NLAFSPATMAHAFLPTM[+15.995]PEDLLAQAR.R | M[+16] | 944.141 | 3 | 2830.409 | 2829.406 | 1 | −0.3 | 4467 | 493.8 | 493.8 | 78.0 | 8.21 |
| 941 | K.NLAFSPATMAHAFLPTMPEDLLAQARR.W | | 743.135 | 4 | 2969.518 | 2969.512 | 0 | 1.7 | 4467 | 546.1 | 546.1 | 546.1 | 7.56 |
| 1303 | R.DGFHLVKDKADR.T | | 467.581 | 3 | 1400.727 | 1400.728 | 0 | −0.5 | 4541 | 710.8 | 710.8 | 710.8 | 9.72 |
| 2683 | K.ADRTQTGHVLGNPQRR.D | | 452.243 | 4 | 1805.949 | 1805.948 | 0 | 0.6 | 4550 | 438.5 | 438.5 | 438.5 | 7.26 |
| 2009 | R.TQTGHVLGNPQRR.D | | 488.599 | 3 | 1463.782 | 1463.783 | 0 | −0.1 | 4553 | 549.4 | 549.4 | 549.4 | 8.65 |
| 2009 | R.TQTGHVLGNPQRR.D | | 488.599 | 3 | 1463.782 | 1463.783 | 0 | −0.1 | 4553 | 518.8 | 518.8 | 518.8 | 7.97 |
| 886 | K.GFLQQHILKDLEQLAK.M | | 627.692 | 3 | 1881.061 | 1881.059 | 0 | 0.7 | 4614 | 614.7 | 614.7 | 614.7 | 10.32 |
| 886 | K.GFLQQHILKDLEQLAK.M | | 471.021 | 4 | 1881.061 | 1881.059 | 0 | 0.7 | 4614 | 461.3 | 461.3 | 461.3 | 8.31 |
| 886 | K.GFLQQHILKDLEQLAK.M | | 941.034 | 2 | 1881.061 | 1881.059 | 0 | 1.0 | 4614 | 556.6 | 556.6 | 556.6 | 7.31 |
| 3522 | K.DLEQLAK.M | | 408.727 | 2 | 816.446 | 816.446 | 0 | 0.2 | 4623 | 334.0 | 120.9 | 120.9 | 4.55 |
| 3522 | K.DLEQLAK.M | | 408.727 | 2 | 816.446 | 816.446 | 0 | 0.2 | 4623 | 353.0 | 91.4 | 91.4 | 4.29 |
| 1043 | K.MLGHSADETIGVVHLVLR.R | | 487.517 | 4 | 1947.048 | 1947.048 | 0 | 0.0 | 4630 | 748.4 | 748.4 | 748.4 | 12.79 |
| 1043 | K.MLGHSADETIGVVHLVLR.R | | 649.688 | 3 | 1947.048 | 1947.048 | 0 | 0.0 | 4630 | 665.2 | 665.2 | 665.2 | 11.79 |
| 1625 | K.M[+15.995]LGHSADETIGVVHLVLRR.L | M[+16] | 424.635 | 5 | 2119.144 | 2119.144 | 0 | 0.1 | 4630 | 588.8 | 588.8 | 588.8 | 9.53 |
| 1043 | K.MLGHSADETIGVVHLVLR.R | | 487.517 | 4 | 1947.048 | 1947.048 | 0 | −0.2 | 4630 | 457.8 | 457.8 | 457.8 | 8.78 |
| 1625 | K.M[+15.995]LGHSADETIGVVHLVLRR.L | M[+16] | 424.635 | 5 | 2119.146 | 2119.144 | 0 | 1.0 | 4630 | 517.0 | 517.0 | 517.0 | 8.72 |
| 819 | K.MLGHSADETIGVVHLVLRR.L | | 421.436 | 5 | 2103.150 | 2103.149 | 0 | 0.4 | 4630 | 504.6 | 504.6 | 504.6 | 8.72 |
| 1625 | K.M[+15.995]LGHSADETIGVVHLVLRR.L | M[+16] | 530.541 | 4 | 2119.143 | 2119.144 | 0 | −0.7 | 4630 | 553.1 | 553.1 | 553.1 | 7.94 |
| 819 | K.MLGHSADETIGVVHLVLRR.L | | 526.543 | 4 | 2103.149 | 2103.149 | 0 | −0.2 | 4630 | 392.1 | 392.1 | 392.1 | 7.27 |
| 819 | K.MLGHSADETIGVVHLVLRR.L | | 701.722 | 3 | 2103.150 | 2103.149 | 0 | 0.3 | 4630 | 345.4 | 345.4 | 345.4 | 4.64 |
| 4265 | R.RLLQEQHQLSSRR.L | | 413.484 | 4 | 1650.915 | 1650.915 | 0 | 0.2 | 4648 | 587.3 | 492.4 | 492.4 | 9.32 |
| 924 | R.RLLQEQHQLSSR.R | | 498.943 | 3 | 1494.814 | 1494.814 | 0 | 0.0 | 4648 | 319.9 | 319.9 | 319.9 | 5.76 |
| 1824 | R.LLQEQHQLSSR.R | | 446.909 | 3 | 1338.713 | 1338.712 | 0 | 0.5 | 4649 | 456.8 | 456.8 | 456.8 | 8.02 |
| 1824 | R.LLQEQHQLSSR.R | | 446.909 | 3 | 1338.712 | 1338.712 | 0 | 0.0 | 4649 | 390.7 | 390.7 | 390.7 | 7.53 |
| 1824 | R.LLQEQHQLSSR.R | | 446.909 | 3 | 1338.712 | 1338.712 | 0 | −0.4 | 4649 | 306.6 | 306.6 | 306.6 | 5.58 |
| 1111 | R.RLLNFDTELSTKEMR.N | | 463.995 | 4 | 1852.959 | 1852.959 | 0 | 0.0 | 4660 | 559.8 | 559.8 | 559.8 | 8.65 |
| 1111 | R.RLLNFDTELSTKEMR.N | | 618.325 | 3 | 1852.960 | 1852.959 | 0 | 0.9 | 4660 | 559.7 | 559.7 | 559.7 | 8.65 |
| 1111 | R.RLLNFDTELSTKEMR.N | | 463.995 | 4 | 1852.959 | 1852.959 | 0 | 0.4 | 4660 | 531.2 | 531.2 | 531.2 | 8.65 |
| 1111 | R.RLLNFDTELSTKEMR.N | | 618.325 | 3 | 1852.960 | 1852.959 | 0 | 0.9 | 4660 | 495.1 | 495.1 | 495.1 | 8.52 |
| 906 | R.RLLNFDTELSTK.E | | 718.891 | 2 | 1436.775 | 1436.774 | 0 | 0.1 | 4660 | 504.0 | 504.0 | 504.0 | 8.23 |
| 906 | R.RLLNFDTELSTK.E | | 479.596 | 3 | 1436.775 | 1436.774 | 0 | 0.1 | 4660 | 451.4 | 451.4 | 451.4 | 8.02 |
| 906 | R.RLLNFDTELSTK.E | | 479.596 | 3 | 1436.774 | 1436.774 | 0 | −0.1 | 4660 | 438.4 | 438.4 | 438.4 | 7.86 |
| 906 | R.RLLNFDTELSTK.E | | 479.596 | 3 | 1436.774 | 1436.774 | 0 | −0.1 | 4660 | 410.0 | 410.0 | 410.0 | 7.86 |
| 1484 | R.RLLN[+0.984]FDTELSTK.E | N[+1] | 479.924 | 3 | 1437.759 | 1437.758 | 0 | 0.3 | 4660 | 433.4 | 349.8 | 349.8 | 6.46 |
| 906 | R.RLLNFDTELSTK.E | | 718.891 | 2 | 1436.775 | 1436.774 | 0 | 0.1 | 4660 | 359.1 | 359.1 | 359.1 | 6.04 |
| 906 | R.RLLNFDTELSTK.E | | 479.596 | 3 | 1436.774 | 1436.774 | 0 | −0.3 | 4660 | 344.0 | 344.0 | 344.0 | 6.04 |
| 821 | R.LLNFDTELSTK.E | | 640.841 | 2 | 1280.674 | 1280.673 | 0 | 0.4 | 4661 | 427.9 | 407.2 | 407.2 | 8.41 |
| 821 | R.LLNFDTELSTK.E | | 640.841 | 2 | 1280.674 | 1280.673 | 0 | 0.7 | 4661 | 519.3 | 519.3 | 519.3 | 8.01 |
| 821 | R.LLNFDTELSTK.E | | 640.841 | 2 | 1280.674 | 1280.673 | 0 | 0.4 | 4661 | 484.1 | 429.2 | 429.2 | 8.01 |
| 821 | R.LLNFDTELSTK.E | | 640.840 | 2 | 1280.673 | 1280.673 | 0 | 0.1 | 4661 | 473.2 | 473.2 | 473.2 | 7.80 |
| 821 | R.LLNFDTELSTK.E | | 640.841 | 2 | 1280.674 | 1280.673 | 0 | 0.4 | 4661 | 446.8 | 388.9 | 388.9 | 7.80 |
| 821 | R.LLNFDTELSTK.E | | 640.840 | 2 | 1280.674 | 1280.673 | 0 | 0.2 | 4661 | 445.3 | 362.8 | 362.8 | 7.80 |
| 821 | R.LLNFDTELSTK.E | | 640.840 | 2 | 1280.674 | 1280.673 | 0 | 0.2 | 4661 | 479.5 | 458.8 | 458.8 | 7.60 |
| 873 | R.LLNFDTELSTKEMR.N | | 848.932 | 2 | 1696.858 | 1696.857 | 0 | 0.0 | 4661 | 403.1 | 403.1 | 403.1 | 7.60 |
| 873 | R.LLNFDTELSTKEMR.N | | 566.291 | 3 | 1696.857 | 1696.857 | 0 | 0.0 | 4661 | 434.7 | 434.7 | 434.7 | 7.38 |
| 821 | R.LLNFDTELSTK.E | | 640.840 | 2 | 1280.674 | 1280.673 | 0 | 0.2 | 4661 | 452.2 | 340.5 | 340.5 | 6.42 |
| 3098 | R.LLN[+0.984]FDTELSTK.E | N[+1] | 641.332 | 2 | 1281.657 | 1281.657 | 0 | −0.2 | 4661 | 401.0 | 324.5 | 324.5 | 6.08 |
| 821 | R.LLNFDTELSTK.E | | 640.841 | 2 | 1280.674 | 1280.673 | 0 | 0.4 | 4661 | 340.8 | 340.8 | 340.8 | 6.04 |
| 821 | R.LLNFDTELSTK.E | | 640.840 | 2 | 1280.674 | 1280.673 | 0 | 0.2 | 4661 | 383.4 | 311.9 | 311.9 | 5.78 |
| 821 | R.LLNFDTELSTK.E | | 427.563 | 3 | 1280.674 | 1280.673 | 0 | 0.5 | 4661 | 347.0 | 232.8 | 232.8 | 5.06 |
| 1321 | R.NNWEKEIAAVISPELEHLDK.T | | 779.070 | 3 | 2335.194 | 2335.193 | 0 | 0.5 | 4675 | 645.0 | 645.0 | 645.0 | 10.53 |
| 1498 | R.N[+0.984]NWEKEIAAVISPELEHLDK.T | N[+1] | 584.800 | 4 | 2336.178 | 2336.177 | 0 | 0.6 | 4675 | 503.1 | 503.1 | 0.0 | 8.72 |
| 1014 | R.NNWEKEIAAVISPELEHLDKTLPTMNNLISQDK.R | | 948.488 | 4 | 3790.932 | 3790.932 | 0 | −0.1 | 4675 | 593.3 | 593.3 | 593.3 | 8.52 |
| 1014 | R.NNWEKEIAAVISPELEHLDKTLPTMNNLISQDK.R | | 758.992 | 5 | 3790.933 | 3790.932 | 0 | 0.2 | 4675 | 587.4 | 587.4 | 587.4 | 8.52 |
| 1014 | R.NNWEKEIAAVISPELEHLDKTLPTMNNLISQDK.R | | 758.992 | 5 | 3790.933 | 3790.932 | 0 | 0.2 | 4675 | 586.3 | 586.3 | 586.3 | 8.52 |
| 1498 | R.N[+0.984]NWEKEIAAVISPELEHLDK.T | N[+1] | 779.398 | 3 | 2336.179 | 2336.177 | 0 | 0.9 | 4675 | 424.8 | 424.8 | 10.3 | 7.59 |
| 1498 | R.N[+0.984]NWEKEIAAVISPELEHLDK.T | N[+1] | 584.800 | 4 | 2336.179 | 2336.177 | 0 | 0.7 | 4675 | 423.5 | 423.5 | 0.0 | 7.59 |
| 1014 | R.NNWEKEIAAVISPELEHLDKTLPTMNNLISQDK.R | | 948.489 | 4 | 3790.933 | 3790.932 | 0 | 0.2 | 4675 | 559.8 | 559.8 | 559.8 | 7.08 |
| 1498 | R.N[+0.984]NWEKEIAAVISPELEHLDK.T | N[+1] | 779.398 | 3 | 2336.179 | 2336.177 | 0 | 0.9 | 4675 | 311.9 | 311.9 | 4.4 | 5.25 |
| 1321 | R.NNWEKEIAAVISPELEHLDK.T | | 1168.101 | 2 | 2335.194 | 2335.193 | 0 | 0.5 | 4675 | 371.7 | 291.0 | 291.0 | 4.99 |
| 1055 | K.EIAAVISPELEHLDKTLPTMNNLISQDKR.I | | 656.152 | 5 | 3276.733 | 3275.730 | 1 | −0.2 | 4680 | 604.5 | 604.5 | 417.1 | 9.52 |
| 1055 | K.EIAAVISPELEHLDKTLPTMNNLISQDKR.I | | 655.952 | 5 | 3275.730 | 3275.730 | 0 | −0.1 | 4680 | 561.0 | 561.0 | 561.0 | 8.52 |
| 933 | K.EIAAVISPELEHLDK.T | | 555.302 | 3 | 1663.890 | 1663.890 | 0 | 0.0 | 4680 | 505.5 | 505.5 | 505.5 | 8.23 |
| 1055 | K.EIAAVISPELEHLDKTLPTMNNLISQDKR.I | | 1092.916 | 3 | 3276.734 | 3275.730 | 1 | 0.0 | 4680 | 622.0 | 622.0 | 340.1 | 8.17 |
| 933 | K.EIAAVISPELEHLDK.T | | 555.301 | 3 | 1663.889 | 1663.890 | 0 | −0.5 | 4680 | 497.4 | 497.4 | 497.4 | 7.86 |
| 3115 | K.EIAAVISPELEHLDKTLPTMN[+0.984]NLISQDKR.I | N[+1] | 656.349 | 5 | 3277.717 | 3276.714 | 1 | −0.3 | 4680 | 458.3 | 458.3 | 46.3 | 6.74 |
| 1055 | K.EIAAVISPELEHLDKTLPTMNNLISQDKR.I | | 819.688 | 4 | 3275.730 | 3275.730 | 0 | −0.1 | 4680 | 440.9 | 440.9 | 440.9 | 6.74 |
| 1597 | K.EIAAVISPELEHLDKTLPTM[+15.995]NNLISQDKR.I | M[+16] | 823.687 | 4 | 3291.728 | 3291.725 | 0 | 0.7 | 4680 | 417.5 | 417.5 | 417.5 | 6.37 |
| 1055 | K.EIAAVISPELEHLDKTLPTMNNLISQDKR.I | | 1092.581 | 3 | 3275.729 | 3275.730 | 0 | −0.5 | 4680 | 592.1 | 592.1 | 592.1 | 6.25 |
| 1055 | K.EIAAVISPELEHLDKTLPTMNNLISQDKR.I | | 1092.581 | 3 | 3275.729 | 3275.730 | 0 | −0.5 | 4680 | 534.0 | 534.0 | 534.0 | 5.58 |
| 882 | K.TLPTMNNLISQDKR.I | | 815.933 | 2 | 1630.859 | 1630.858 | 0 | 0.4 | 4695 | 535.9 | 535.9 | 535.9 | 8.65 |
| 882 | K.TLPTMNNLISQDKR.I | | 815.933 | 2 | 1630.858 | 1630.858 | 0 | 0.1 | 4695 | 523.3 | 523.3 | 523.3 | 8.65 |
| 882 | K.TLPTMNNLISQDKR.I | | 544.291 | 3 | 1630.858 | 1630.858 | 0 | −0.1 | 4695 | 404.3 | 404.3 | 404.3 | 7.38 |
| 1574 | K.TLPTM[+15.995]NNLISQDKR.I | M[+16] | 549.622 | 3 | 1646.853 | 1646.853 | 0 | −0.2 | 4695 | 401.2 | 401.2 | 401.2 | 7.38 |
| 882 | K.TLPTMNNLISQDKR.I | | 544.291 | 3 | 1630.858 | 1630.858 | 0 | −0.2 | 4695 | 376.2 | 376.2 | 376.2 | 7.27 |
| 882 | K.TLPTMNNLISQDKR.I | | 544.291 | 3 | 1630.858 | 1630.858 | 0 | −0.1 | 4695 | 369.9 | 369.9 | 369.9 | 7.06 |
| 1574 | K.TLPTM[+15.995]NNLISQDKR.I | M[+16] | 549.622 | 3 | 1646.852 | 1646.853 | 0 | −0.6 | 4695 | 463.6 | 463.6 | 463.6 | 6.63 |
| 882 | K.TLPTMNNLISQDKR.I | | 544.291 | 3 | 1630.858 | 1630.858 | 0 | −0.2 | 4695 | 317.7 | 317.7 | 317.7 | 5.50 |
| 882 | K.TLPTMNNLISQDKR.I | | 544.291 | 3 | 1630.858 | 1630.858 | 0 | −0.3 | 4695 | 314.3 | 314.3 | 314.3 | 5.50 |
| 882 | K.TLPTMNNLISQDKR.I | | 544.291 | 3 | 1630.857 | 1630.858 | 0 | −0.4 | 4695 | 404.8 | 354.8 | 354.8 | 5.29 |
| 1838 | R.ISSNPVAK.I | | 408.235 | 2 | 815.462 | 815.462 | 0 | 0.2 | 4709 | 347.6 | 347.6 | 347.6 | 5.58 |
| 1838 | R.ISSNPVAK.I | | 408.235 | 2 | 815.462 | 815.462 | 0 | 0.0 | 4709 | 302.6 | 302.6 | 302.6 | 5.58 |
| 1172 | K.IIYGDPVTFLPHLPR.K | | 579.995 | 3 | 1737.969 | 1737.969 | 0 | 0.2 | 4717 | 682.9 | 682.9 | 682.9 | 11.58 |
| 1172 | K.IIYGDPVTFLPHLPR.K | | 579.994 | 3 | 1737.968 | 1737.969 | 0 | −0.2 | 4717 | 656.5 | 656.5 | 656.5 | 11.58 |
| 1172 | K.IIYGDPVTFLPHLPR.K | | 869.488 | 2 | 1737.969 | 1737.969 | 0 | 0.2 | 4717 | 603.4 | 603.4 | 603.4 | 10.58 |
| 1172 | K.IIYGDPVTFLPHLPR.K | | 435.247 | 4 | 1737.967 | 1737.969 | 0 | −0.7 | 4717 | 672.9 | 672.9 | 672.9 | 10.13 |
| 846 | K.IIYGDPVTFLPHLPRK.S | | 467.271 | 4 | 1866.064 | 1866.064 | 0 | 0.1 | 4717 | 599.9 | 599.9 | 599.9 | 9.32 |
| 1172 | K.IIYGDPVTFLPHLPR.K | | 579.994 | 3 | 1737.969 | 1737.969 | 0 | 0.1 | 4717 | 499.9 | 499.9 | 499.9 | 8.78 |
| 846 | K.IIYGDPVTFLPHLPRK.S | | 467.271 | 4 | 1866.064 | 1866.064 | 0 | −0.1 | 4717 | 500.4 | 500.4 | 500.4 | 7.97 |
| 846 | K.IIYGDPVTFLPHLPRK.S | | 467.272 | 4 | 1866.064 | 1866.064 | 0 | 0.2 | 4717 | 447.0 | 447.0 | 447.0 | 7.76 |
| 1172 | K.IIYGDPVTFLPHLPR.K | | 579.994 | 3 | 1737.968 | 1737.969 | 0 | −0.1 | 4717 | 336.9 | 336.9 | 336.9 | 6.04 |
| 846 | K.IIYGDPVTFLPHLPRK.S | | 933.535 | 2 | 1866.062 | 1866.064 | 0 | −0.7 | 4717 | 402.7 | 402.7 | 402.7 | 5.89 |
| 2008 | R.KRITVEYLQHIVEQK.N | | 471.773 | 4 | 1884.070 | 1884.070 | 0 | 0.1 | 4745 | 542.1 | 542.1 | 542.1 | 8.65 |
| 2008 | R.KRITVEYLQHIVEQK.N | | 471.773 | 4 | 1884.070 | 1884.070 | 0 | 0.1 | 4745 | 507.1 | 507.1 | 507.1 | 7.97 |
| 2008 | R.KRITVEYLQHIVEQK.N | | 471.773 | 4 | 1884.070 | 1884.070 | 0 | 0.1 | 4745 | 344.3 | 344.3 | 344.3 | 5.98 |
| 1159 | R.ITVEYLQHIVEQK.N | | 533.963 | 3 | 1599.874 | 1599.874 | 0 | −0.3 | 4747 | 670.9 | 670.9 | 670.9 | 11.58 |
| 1512 | R.ITVEYLQHIVEQKN[+0.984]GKER.V | N[+1] | 547.046 | 4 | 2185.161 | 2185.161 | 0 | −0.2 | 4747 | 434.6 | 434.6 | 257.4 | 8.02 |
| 1159 | R.ITVEYLQHIVEQK.N | | 533.963 | 3 | 1599.873 | 1599.874 | 0 | −0.7 | 4747 | 522.8 | 522.8 | 522.8 | 7.45 |
| 863 | R.VPILWHFLQK.E | | 427.589 | 3 | 1280.751 | 1280.751 | 0 | −0.1 | 4765 | 388.7 | 388.7 | 388.7 | 7.53 |
| 879 | R.VPILWHFLQKEAELR.L | | 470.520 | 4 | 1879.059 | 1879.059 | 0 | 0.2 | 4765 | 371.2 | 371.2 | 371.2 | 7.49 |
| 853 | K.FLPEILALQR.D | | 600.361 | 2 | 1199.714 | 1199.715 | 0 | −0.3 | 4783 | 470.1 | 470.1 | 470.1 | 7.60 |
| 853 | K.FLPEILALQR.D | | 600.361 | 2 | 1199.715 | 1199.715 | 0 | 0.2 | 4783 | 464.3 | 464.3 | 464.3 | 7.60 |
| 853 | K.FLPEILALQR.D | | 400.576 | 3 | 1199.714 | 1199.715 | 0 | −0.3 | 4783 | 455.6 | 455.6 | 455.6 | 7.06 |
| 853 | K.FLPEILALQR.D | | 600.361 | 2 | 1199.715 | 1199.715 | 0 | 0.4 | 4783 | 358.6 | 358.6 | 358.6 | 5.86 |
| 853 | K.FLPEILALQR.D | | 400.911 | 3 | 1200.717 | 1199.715 | 1 | −0.6 | 4783 | 395.0 | 395.0 | 395.0 | 4.09 |
| 1019 | R.DLVKQFQNVQQVEYSSIR.G | | 1091.068 | 2 | 2181.129 | 2181.130 | 0 | −0.3 | 4793 | 611.4 | 596.2 | 596.2 | 10.53 |
| 1019 | R.DLVKQFQNVQQVEYSSIR.G | | 1091.069 | 2 | 2181.130 | 2181.130 | 0 | 0.0 | 4793 | 593.0 | 518.6 | 518.6 | 9.53 |
| 1019 | R.DLVKQFQNVQQVEYSSIR.G | | 727.715 | 3 | 2181.132 | 2181.130 | 0 | 0.8 | 4793 | 481.2 | 481.2 | 481.2 | 8.18 |
| 1019 | R.DLVKQFQNVQQVEYSSIR.G | | 727.715 | 3 | 2181.132 | 2181.130 | 0 | 0.8 | 4793 | 434.5 | 434.5 | 434.5 | 7.81 |
| 1019 | R.DLVKQFQNVQQVEYSSIR.G | | 727.716 | 3 | 2181.132 | 2181.130 | 0 | 1.1 | 4793 | 427.5 | 427.5 | 427.5 | 7.59 |
| 1019 | R.DLVKQFQNVQQVEYSSIR.G | | 727.716 | 3 | 2181.132 | 2181.130 | 0 | 1.1 | 4793 | 319.4 | 319.4 | 319.4 | 5.71 |
| 818 | K.QFQNVQQVEYSSIR.G | | 863.432 | 2 | 1725.856 | 1725.856 | 0 | 0.1 | 4797 | 626.8 | 626.8 | 626.8 | 10.58 |
| 818 | K.QFQNVQQVEYSSIR.G | | 863.431 | 2 | 1725.855 | 1725.856 | 0 | 0.0 | 4797 | 474.9 | 474.9 | 474.9 | 8.57 |
| 1050 | K.QFQNVQQVEYSSIRGFLSK.H | | 753.390 | 3 | 2258.156 | 2258.156 | 0 | −0.1 | 4797 | 413.3 | 413.3 | 413.3 | 7.59 |
| 818 | K.QFQNVQQVEYSSIR.G | | 575.957 | 3 | 1725.856 | 1725.856 | 0 | 0.4 | 4797 | 431.8 | 431.8 | 431.8 | 7.10 |
| 818 | K.QFQNVQQVEYSSIR.G | | 575.957 | 3 | 1725.855 | 1725.856 | 0 | −0.3 | 4797 | 381.5 | 381.5 | 381.5 | 6.99 |
| 925 | R.ITVFLSTWNK.L | | 604.837 | 2 | 1208.667 | 1208.667 | 0 | −0.1 | 4829 | 402.4 | 402.4 | 402.4 | 7.44 |
| 3272 | R.SLETN[+0.984]GEINLPKDYC[+57.021]STDLDLDTEFEI | C[+57], | 1205.250 | 3 | 3613.737 | 3611.731 | 2 | −0.3 | 4842 | 755.0 | 594.1 | 83.2 | 12.85 |
| | LLPR.R | N[+1] | | | | | | | | | | | |
| 1720 | R.SLETNGEINLPKDYC[+57.021]STDLDLDTEFEILLPR.R | C[+57] | 1204.255 | 3 | 3610.750 | 3610.747 | 0 | 0.9 | 4842 | 681.2 | 681.2 | 681.2 | 10.85 |
| 1146 | R.SLETNGEINLPK.D | | 657.849 | 2 | 1314.690 | 1314.690 | 0 | 0.2 | 4842 | 605.5 | 605.5 | 605.5 | 10.58 |
| 1504 | R.SLETN[+0.984]GEINLPK.D | N[+1] | 658.341 | 2 | 1315.675 | 1315.674 | 0 | 0.6 | 4842 | 463.2 | 463.2 | 45.2 | 7.80 |
| 1720 | R.SLETNGEINLPKDYC[+57.021]STDLDLDTEFEILLPR.R | C[+57] | 903.444 | 4 | 3610.752 | 3610.747 | 0 | 1.4 | 4842 | 504.9 | 485.8 | 485.8 | 6.54 |
| 1720 | R.SLETNGEINLPKDYC[+57.021]STDLDLDTEFEILLPR.R | C[+57] | 903.442 | 4 | 3610.746 | 3610.747 | 0 | −0.2 | 4842 | 496.6 | 496.6 | 496.6 | 6.54 |
| 1720 | R.SLETNGEINLPKDYC[+57.021]STDLDLDTEFEILLPR.R | C[+57] | 1204.257 | 3 | 3610.757 | 3610.747 | 0 | 2.8 | 4842 | 542.9 | 395.5 | 395.5 | 5.91 |
| 3271 | R.SLETNGEIN[+0.984]LPKDYC[+57.021]STDLDLDTEFEI | C[+57], | 1204.581 | 3 | 3611.728 | 3611.731 | 0 | −0.8 | 4842 | 445.6 | 385.7 | 13.2 | 5.50 |
| | LLPR.R | N[+1] | | | | | | | | | | | |
| 4656 | R.SLETNGEIN[+0.984]LPKDY[+79.966]C[+57.021]STD | C[+57], | 924.176 | 4 | 3693.683 | 3691.697 | 2 | −5.6 | 4842 | 302.7 | 290.1 | 42.4 | 2.46 |
| | LDLDTEFEILLPR.R | N[+1], | | | | | | | | | | | |
| | | Y[+80] | | | | | | | | | | | |
| 1715 | R.GLGLC[+57.021]ATALVSYLIR.L | C[+57] | 536.305 | 3 | 1606.899 | 1606.899 | 0 | 0.5 | 4875 | 524.1 | 524.1 | 524.1 | 8.37 |
| 1819 | R.LHNEIVYAVEKLSK.E | | 411.485 | 4 | 1642.916 | 1642.916 | 0 | 0.1 | 4890 | 496.2 | 496.2 | 496.2 | 8.52 |
| 1819 | R.LHNEIVYAVEKLSK.E | | 548.310 | 3 | 1642.916 | 1642.916 | 0 | −0.4 | 4890 | 501.0 | 501.0 | 501.0 | 7.97 |
| 1246 | R.LHNEIVYAVEK.L | | 438.907 | 3 | 1314.705 | 1314.705 | 0 | −0.2 | 4890 | 449.9 | 449.9 | 449.9 | 7.80 |
| 1246 | R.LHNEIVYAVEK.L | | 438.907 | 3 | 1314.705 | 1314.705 | 0 | −0.2 | 4890 | 421.4 | 421.4 | 421.4 | 7.64 |
| 1246 | R.LHNEIVYAVEK.L | | 657.856 | 2 | 1314.705 | 1314.705 | 0 | −0.2 | 4890 | 372.9 | 372.9 | 372.9 | 7.53 |
| 4342 | R.ETVQEFDLEKIQR.Q | | 817.923 | 2 | 1634.838 | 1634.838 | 0 | −0.1 | 4946 | 603.2 | 603.2 | 603.2 | 10.32 |
| 1004 | R.LSLKGIPTLVYR.H | | 680.422 | 2 | 1359.836 | 1359.836 | 0 | 0.0 | 4971 | 681.3 | 681.3 | 681.3 | 11.32 |
| 1004 | R.LSLKGIPTLVYR.H | | 453.950 | 3 | 1359.836 | 1359.836 | 0 | 0.0 | 4971 | 553.5 | 553.5 | 553.5 | 8.65 |
| 1004 | R.LSLKGIPTLVYR.H | | 453.950 | 3 | 1359.836 | 1359.836 | 0 | −0.1 | 4971 | 473.5 | 473.5 | 473.5 | 7.76 |
| 1004 | R.LSLKGIPTLVYR.H | | 453.950 | 3 | 1359.836 | 1359.836 | 0 | 0.2 | 4971 | 467.1 | 467.1 | 467.1 | 7.76 |
| 1912 | K.GIPTLVYR.H | | 459.774 | 2 | 918.541 | 918.541 | 0 | 0.4 | 4975 | 347.3 | 347.3 | 347.3 | 5.86 |
| 966 | R.HDWNYEHLFMDIK.N | | 583.268 | 3 | 1747.790 | 1747.790 | 0 | 0.1 | 4983 | 576.6 | 576.6 | 576.6 | 9.03 |
| 966 | R.HDWNYEHLFMDIK.N | | 583.268 | 3 | 1747.790 | 1747.790 | 0 | 0.4 | 4983 | 557.4 | 557.4 | 557.4 | 8.91 |
| 2192 | R.HDWNYEHLFMDIKNK.M | | 498.237 | 4 | 1989.928 | 1989.928 | 0 | −0.1 | 4983 | 455.0 | 455.0 | 455.0 | 8.31 |
| 966 | R.HDWNYEHLFMDIK.N | | 583.268 | 3 | 1747.790 | 1747.790 | 0 | 0.2 | 4983 | 491.8 | 491.8 | 491.8 | 8.23 |
| 966 | R.HDWNYEHLFMDIK.N | | 874.399 | 2 | 1747.790 | 1747.790 | 0 | 0.0 | 4983 | 524.1 | 524.1 | 524.1 | 7.57 |
| 1565 | R.HDWNYEHLFM[+15.995]DIK.N | M[+16] | 588.600 | 3 | 1763.785 | 1763.785 | 0 | 0.0 | 4983 | 406.7 | 406.7 | 406.7 | 7.44 |
| 1611 | R.HDWNYEHLFM[+15.995]DIKNK.M | M[+16] | 502.236 | 4 | 2005.920 | 2005.923 | 0 | −1.1 | 4983 | 501.0 | 501.0 | 501.0 | 7.05 |
| 1020 | K.HTIALWQFLSAHKSEQLLR.L | | 570.317 | 4 | 2278.247 | 2278.246 | 0 | 0.5 | 5074 | 496.8 | 496.8 | 496.8 | 8.18 |
| 2095 | K.HTIALWQFLSAHK.S | | 517.953 | 3 | 1551.843 | 1551.843 | 0 | 0.1 | 5074 | 454.6 | 454.6 | 454.6 | 7.60 |
| 903 | R.LHKEPFGEISSR.Y | | 467.249 | 3 | 1399.733 | 1399.733 | 0 | 0.3 | 5093 | 598.9 | 598.9 | 598.9 | 9.32 |
| 903 | R.LHKEPFGEISSR.Y | | 467.250 | 3 | 1399.734 | 1399.733 | 0 | 0.7 | 5093 | 599.4 | 599.4 | 599.4 | 8.77 |
| 903 | R.LHKEPFGEISSR.Y | | 467.249 | 3 | 1399.733 | 1399.733 | 0 | 0.1 | 5093 | 576.0 | 576.0 | 576.0 | 8.77 |
| 903 | R.LHKEPFGEISSR.Y | | 467.249 | 3 | 1399.733 | 1399.733 | 0 | 0.2 | 5093 | 586.5 | 586.5 | 586.5 | 8.55 |
| 903 | R.LHKEPFGEISSR.Y | | 467.249 | 3 | 1399.733 | 1399.733 | 0 | −0.2 | 5093 | 555.9 | 555.9 | 555.9 | 8.10 |
| 903 | R.LHKEPFGEISSR.Y | | 467.249 | 3 | 1399.733 | 1399.733 | 0 | 0.2 | 5093 | 555.6 | 555.6 | 555.6 | 8.10 |
| 903 | R.LHKEPFGEISSR.Y | | 467.249 | 3 | 1399.733 | 1399.733 | 0 | 0.1 | 5093 | 483.1 | 483.1 | 483.1 | 7.97 |
| 903 | R.LHKEPFGEISSR.Y | | 467.249 | 3 | 1399.732 | 1399.733 | 0 | −0.4 | 5093 | 512.9 | 512.9 | 512.9 | 7.75 |
| 903 | R.LHKEPFGEISSR.Y | | 467.249 | 3 | 1399.734 | 1399.733 | 0 | 0.5 | 5093 | 402.3 | 402.3 | 402.3 | 7.38 |
| 903 | R.LHKEPFGEISSR.Y | | 467.249 | 3 | 1399.732 | 1399.733 | 0 | −0.3 | 5093 | 362.5 | 362.5 | 362.5 | 7.06 |
| 903 | R.LHKEPFGEISSR.Y | | 467.249 | 3 | 1399.734 | 1399.733 | 0 | 0.6 | 5093 | 346.6 | 346.6 | 346.6 | 5.98 |
| 903 | R.LHKEPFGEISSR.Y | | 700.370 | 2 | 1399.733 | 1399.733 | 0 | −0.3 | 5093 | 346.7 | 346.7 | 346.7 | 4.44 |
| 2609 | R.YKADLSPENAK.L | | 618.317 | 2 | 1235.627 | 1235.627 | 0 | 0.1 | 5105 | 467.8 | 467.8 | 467.8 | 7.54 |
| 3259 | K.LLSTFLNQTGLDAFLLELHEM[+15.995]IILK.L | M[+16] | 963.534 | 3 | 2888.587 | 2888.584 | 0 | 1.0 | 5116 | 821.8 | 821.8 | 821.8 | 14.78 |
| 3259 | K.LLSTFLNQTGLDAFLLELHEM[+15.995]IILK.L | M[+16] | 963.534 | 3 | 2888.587 | 2888.584 | 0 | 0.8 | 5116 | 781.8 | 781.8 | 781.8 | 13.79 |
| 3259 | K.LLSTFLNQTGLDAFLLELHEM[+15.995]IILK.L | M[+16] | 722.902 | 4 | 2888.584 | 2888.584 | 0 | 0.0 | 5116 | 790.4 | 790.4 | 790.4 | 13.25 |
| 1450 | K.LLSTFLNQTGLDAFLLELHEMIILK.L | | 958.202 | 3 | 2872.591 | 2872.589 | 0 | 0.4 | 5116 | 658.8 | 658.8 | 658.8 | 11.79 |
| 1450 | K.LLSTFLNQTGLDAFLLELHEMIILK.L | | 718.904 | 4 | 2872.593 | 2872.589 | 0 | 1.2 | 5116 | 673.6 | 673.6 | 673.6 | 11.25 |
| 3723 | K.LKNPQTQTEER.F | | 672.350 | 2 | 1343.692 | 1343.691 | 0 | 0.4 | 5141 | 408.7 | 408.7 | 408.7 | 7.18 |
| 3723 | K.LKNPQTQTEER.F | | 448.568 | 3 | 1343.691 | 1343.691 | 0 | −0.5 | 5141 | 370.3 | 370.3 | 370.3 | 6.35 |
| 1786 | R.FRPQWSLRDTLVSYMQTK.E | | 564.796 | 4 | 2256.160 | 2256.159 | 0 | 0.4 | 5152 | 391.1 | 391.1 | 391.1 | 7.27 |
| 2126 | R.FRPQWSLR.D | | 545.301 | 2 | 1089.595 | 1089.595 | 0 | −0.3 | 5152 | 398.0 | 398.0 | 398.0 | 7.14 |
| 861 | R.DTLVSYMQTK.E | | 593.295 | 2 | 1185.582 | 1185.582 | 0 | 0.2 | 5160 | 503.9 | 442.6 | 442.6 | 8.23 |
| 1540 | R.DTLVSYM[+15.995]QTK.E | M[+16] | 601.292 | 2 | 1201.577 | 1201.577 | 0 | 0.2 | 5160 | 491.8 | 491.8 | 491.8 | 8.23 |
| 861 | R.DTLVSYMQTK.E | | 593.295 | 2 | 1185.582 | 1185.582 | 0 | 0.1 | 5160 | 454.4 | 454.4 | 454.4 | 7.80 |
| 861 | R.DTLVSYMQTK.E | | 593.295 | 2 | 1185.582 | 1185.582 | 0 | 0.1 | 5160 | 379.5 | 379.5 | 379.5 | 7.53 |
| 861 | R.DTLVSYMQTK.E | | 593.294 | 2 | 1185.580 | 1185.582 | 0 | −1.9 | 5160 | 318.8 | 318.8 | 318.8 | 4.26 |
| 4036 | K.PIPNPLLGLDSTASKDNTVPLKLIALLANGEFHSGEQLGETLGM | | 963.716 | 5 | 4814.550 | 4814.540 | 0 | 2.1 | 5211 | 483.7 | 410.9 | 410.9 | 5.06 |
| | SR.A | | | | | | | | | | | | |
| 1144 | K.DNTVPLKLIALLANGEFHSGEQLGETLGMSR.A | | 1104.241 | 3 | 3310.710 | 3310.710 | 0 | −0.1 | 5226 | 822.4 | 822.4 | 822.4 | 13.85 |
| 1144 | K.DNTVPLKLIALLANGEFHSGEQLGETLGMSR.A | | 1104.240 | 3 | 3310.706 | 3310.710 | 0 | −1.2 | 5226 | 759.5 | 759.5 | 759.5 | 11.94 |
| 1144 | K.DNTVPLKLIALLANGEFHSGEQLGETLGMSR.A | | 828.433 | 4 | 3310.711 | 3310.710 | 0 | 0.4 | 5226 | 728.6 | 683.4 | 683.4 | 11.85 |
| 1144 | K.DNTVPLKLIALLANGEFHSGEQLGETLGMSR.A | | 828.433 | 4 | 3310.709 | 3310.710 | 0 | −0.4 | 5226 | 691.1 | 691.1 | 691.1 | 10.85 |
| 1144 | K.DNTVPLKLIALLANGEFHSGEQLGETLGMSR.A | | 1104.574 | 3 | 3311.706 | 3310.710 | 1 | −2.2 | 5226 | 670.7 | 670.7 | 68.7 | 9.94 |
| 1575 | K.DNTVPLKLIALLANGEFHSGEQLGETLGM[+15.995]SR.A | M[+16] | 832.432 | 4 | 3326.708 | 3326.705 | 0 | 0.8 | 5226 | 574.1 | 520.5 | 520.5 | 8.85 |
| 1144 | K.DNTVPLKLIALLANGEFHSGEQLGETLGMSR.A | | 662.948 | 5 | 3310.709 | 3310.710 | 0 | −0.4 | 5226 | 471.2 | 471.2 | 471.2 | 6.76 |
| 1144 | K.DNTVPLKLIALLANGEFHSGEQLGETLGMSR.A | | 662.947 | 5 | 3310.708 | 3310.710 | 0 | −0.7 | 5226 | 493.9 | 493.9 | 493.9 | 6.59 |
| 1144 | K.DNTVPLKLIALLANGEFHSGEQLGETLGMSR.A | | 662.948 | 5 | 3310.711 | 3310.710 | 0 | 0.2 | 5226 | 399.9 | 399.9 | 399.9 | 6.26 |
| 874 | K.LIALLANGEFHSGEQLGETLGMSR.A | | 848.436 | 3 | 2543.292 | 2543.292 | 0 | 0.0 | 5233 | 745.4 | 646.4 | 646.4 | 12.79 |
| 874 | K.LIALLANGEFHSGEQLGETLGMSR.A | | 848.435 | 3 | 2543.291 | 2543.292 | 0 | −0.7 | 5233 | 740.4 | 691.3 | 691.3 | 11.87 |
| 1515 | K.LIALLAN[+0.984]GEFHSGEQLGETLGMSR.A | N[+1] | 848.764 | 3 | 2544.277 | 2544.276 | 0 | 0.2 | 5233 | 662.7 | 582.4 | 282.5 | 11.79 |
| 874 | K.LIALLANGEFHSGEQLGETLGMSR.A | | 848.436 | 3 | 2543.293 | 2543.292 | 0 | 0.4 | 5233 | 637.7 | 515.1 | 515.1 | 10.79 |
| 1874 | K.LIALLANGEFHSGEQLGETLGMSR.A | | 848.436 | 3 | 2543.292 | 2543.292 | 0 | −0.2 | 5233 | 620.8 | 620.8 | 620.8 | 10.79 |
| 874 | K.LIALLANGEFHSGEQLGETLGMSR.A | | 848.436 | 3 | 2543.293 | 2543.292 | 0 | 0.1 | 5233 | 569.6 | 469.3 | 469.3 | 9.24 |
| 874 | K.LIALLANGEFHSGEQLGETLGMSR.A | | 636.579 | 4 | 2543.293 | 2543.292 | 0 | 0.5 | 5233 | 508.5 | 508.5 | 508.5 | 8.45 |
| 874 | K.LIALLANGEFHSGEQLGETLGMSR.A | | 636.579 | 4 | 2543.293 | 2543.292 | 0 | 0.5 | 5233 | 472.5 | 407.7 | 407.7 | 8.24 |
| 874 | K.LIALLANGEFHSGEQLGETLGMSR.A | | 848.435 | 3 | 2543.291 | 2543.292 | 0 | −0.5 | 5233 | 544.0 | 434.2 | 434.2 | 8.20 |
| 1515 | K.LIALLAN[+0.984]GEFHSGEQLGETLGMSR.A | N[+] | 848.763 | 3 | 2544.276 | 2544.276 | 0 | −0.2 | 5233 | 418.9 | 418.9 | 68.1 | 8.07 |
| 874 | K.LIALLANGEFHSGEQLGETLGMSR.A | | 848.436 | 3 | 2543.293 | 2543.292 | 0 | 0.1 | 5233 | 316.1 | 316.1 | 316.1 | 5.97 |
| 852 | R.AAINKHIQTLR.D | | 422.255 | 3 | 1264.749 | 1264.748 | 0 | 0.4 | 5257 | 499.7 | 499.7 | 499.7 | 8.52 |
| 852 | R.AAINKHIQTLR.D | | 422.254 | 3 | 1264.749 | 1264.748 | 0 | 0.1 | 5257 | 414.7 | 414.7 | 414.7 | 7.60 |
| 1796 | R.AAINKHIQTLRDWGVDVFTVPGK.G | | 642.104 | 4 | 2565.394 | 2565.394 | 0 | 0.3 | 5257 | 399.8 | 399.8 | 399.8 | 7.36 |
| 1796 | R.AAINKHIQTLRDWGVDVFTVPGK.G | | 642.104 | 4 | 2565.394 | 2565.394 | 0 | 0.0 | 5257 | 340.5 | 340.5 | 340.5 | 5.65 |
| 826 | K.HIQTLRDWGVDVFTVPGKGYSLPEPIPLLNAK.Q | | 890.987 | 4 | 3560.927 | 3560.926 | 0 | 0.1 | 5262 | 627.4 | 627.4 | 627.4 | 9.52 |
| 826 | K.HIQTLRDWGVDVFTVPGKGYSLPEPIPLLNAK.Q | | 712.991 | 5 | 3560.926 | 3560.926 | 0 | −0.2 | 5262 | 462.1 | 462.1 | 462.1 | 7.51 |
| 826 | K.HIQTLRDWGVDVFTVPGKGYSLPEPIPLLNAK.Q | | 890.988 | 4 | 3560.929 | 3560.926 | 0 | 0.7 | 5262 | 520.3 | 520.3 | 520.3 | 7.30 |
| 1810 | K.HIQTLRDWGVDVFTVPGK.G | | 690.037 | 3 | 2068.098 | 2068.097 | 0 | 0.2 | 5262 | 372.4 | 372.4 | 372.4 | 7.27 |
| 826 | K.HIQTLRDWGVDVFTVPGKGYSLPEPIPLLNAK.Q | | 1187.648 | 3 | 3560.931 | 3560.926 | 0 | 1.2 | 5262 | 585.5 | 585.5 | 585.5 | 7.17 |
| 826 | K.HIQTLRDWGVDVFTVPGKGYSLPEPIPLLNAK.Q | | 712.991 | 5 | 3560.927 | 3560.926 | 0 | 0.3 | 5262 | 431.7 | 431.7 | 431.7 | 6.79 |
| 3102 | K.HIQTLRDWGVDVFTVPGKGYSLPEPIPLLN[+0.984]AK.Q | IN[+1] | 713.189 | 5 | 3561.913 | 3561.910 | 0 | 0.8 | 5262 | 407.1 | 407.1 | 199.3 | 6.57 |
| 826 | K.HIQTLRDWGVDVFTVPGKGYSLPEPIPLLNAK.Q | | 712.991 | 5 | 3560.926 | 3560.926 | 0 | 0.0 | 5262 | 353.9 | 353.9 | 353.9 | 4.79 |
| 1810 | K.HIQTLRDWGVDVFTVPGK.G | | 1034.552 | 2 | 2068.097 | 2068.097 | 0 | −0.1 | 5262 | 325.5 | 325.5 | 325.5 | 4.64 |
| 826 | K.HIQTLRDWGVDVFTVPGKGYSLPEPIPLLNAK.Q | | 594.327 | 6 | 3560.927 | 3560.926 | 0 | 0.2 | 5262 | 324.8 | 324.8 | 324.8 | 4.63 |
| 1109 | R.DWGVDVFTVPGKGYSLPEPIPLLNAK.Q | | 938.169 | 3 | 2812.492 | 2812.492 | 0 | 0.1 | 5268 | 863.5 | 863.5 | 863.5 | 15.48 |
| 1510 | R.DWGVDVFTVPGKGYSLPEPIPLLN[+0.984]AK.Q | N[+1] | 938.498 | 3 | 2813.479 | 2813.476 | 0 | 1.2 | 5268 | 825.8 | 825.8 | 592.9 | 14.54 |
| 1109 | R.DWGVDVFTVPGKGYSLPEPIPLLNAK.Q | | 938.170 | 3 | 2812.496 | 2812.492 | 0 | 1.3 | 5268 | 793.5 | 793.5 | 793.5 | 13.53 |
| 1109 | R.DWGVDVFTVPGKGYSLPEPIPLLNAK.Q | | 1406.752 | 2 | 2812.497 | 2812.492 | 0 | 1.6 | 5268 | 703.7 | 703.7 | 703.7 | 12.53 |
| 1510 | R.DWGVDVFTVPGKGYSLPEPIPLLN[+0.984]AK.Q | N[+1] | 938.497 | 3 | 2813.478 | 2813.476 | 0 | 0.6 | 5268 | 698.1 | 698.1 | 322.3 | 11.53 |
| 1109 | R.DWGVDVFTVPGKGYSLPEPIPLLNAK.Q | | 938.169 | 3 | 2812.493 | 2812.492 | 0 | 0.3 | 5268 | 665.8 | 665.8 | 665.8 | 11.53 |
| 931 | R.DWGVDVFTVPGK.G | | 660.335 | 2 | 1319.663 | 1319.663 | 0 | 0.2 | 5268 | 521.0 | 521.0 | 521.0 | 8.91 |
| 931 | R.DWGVDVFTVPGK.G | | 660.336 | 2 | 1319.665 | 1319.663 | 0 | 1.3 | 5268 | 521.4 | 521.4 | 521.4 | 8.36 |
| 1109 | R.DWGVDVFTVPGKGYSLPEPIPLLNAK.Q | | 703.879 | 4 | 2812.494 | 2812.492 | 0 | 0.7 | 5268 | 548.3 | 548.3 | 548.3 | 8.32 |
| 931 | R.DWGVDVFTVPGK.G | | 660.336 | 2 | 1319.664 | 1319.663 | 0 | 0.5 | 5268 | 390.9 | 390.9 | 390.9 | 7.53 |
| 931 | R.DWGVDVFTVPGK.G | | 440.559 | 3 | 1319.663 | 1319.663 | 0 | 0.2 | 5268 | 377.6 | 377.6 | 377.6 | 6.60 |
| 950 | K.GYSLPEPIPLLNAK.Q | | 756.427 | 2 | 1511.846 | 1511.847 | 0 | −0.3 | 5280 | 664.7 | 664.7 | 664.7 | 11.58 |
| 950 | K.GYSLPEPIPLLNAK.Q | | 504.620 | 3 | 1511.846 | 1511.847 | 0 | −0.4 | 5280 | 595.2 | 595.2 | 595.2 | 9.04 |
| 950 | K.GYSLPEPIPLLNAK.Q | | 756.427 | 2 | 1511.847 | 1511.847 | 0 | −0.1 | 5280 | 534.9 | 534.9 | 534.9 | 8.91 |
| 950 | K.GYSLPEPIPLLNAK.Q | | 756.427 | 2 | 1511.846 | 1511.847 | 0 | −0.4 | 5280 | 473.9 | 473.9 | 473.9 | 8.57 |
| 950 | K.GYSLPEPIPLLNAK.Q | | 756.427 | 2 | 1511.846 | 1511.847 | 0 | 0.3 | 5280 | 440.5 | 440.5 | 440.5 | 8.57 |
| 950 | K.GYSLPEPIPLLNAK.Q | | 756.427 | 2 | 1511.847 | 1511.847 | 0 | −0.1 | 5280 | 406.2 | 406.2 | 406.2 | 8.41 |
| 950 | K.GYSLPEPIPLLNAK.Q | | 756.427 | 2 | 1511.847 | 1511.847 | 0 | 0.1 | 5280 | 395.9 | 395.9 | 395.9 | 7.75 |
| 950 | K.GYSLPEPIPLLNAK.Q | | 504.620 | 3 | 1511.844 | 1511.847 | 0 | −1.6 | 5280 | 518.9 | 518.9 | 518.9 | 7.32 |
| 950 | K.GYSLPEPIPLLNAK.Q | | 756.427 | 2 | 1511.846 | 1511.847 | 0 | −0.5 | 5280 | 412.0 | 412.0 | 412.0 | 6.94 |
| 3127 | K.QILGQLDGGSVAVLPVVDSTN[+0.984]QYLLDRIGELK.S | N[+1] | 1137.953 | 3 | 3411.845 | 3411.837 | 0 | 2.4 | 5294 | 949.6 | 949.6 | 502.2 | 14.56 |
| 964 | K.QILGQLDGGSVAVLPVVDSTNQYLLDRIGELK.S | | 1137.626 | 3 | 3410.863 | 3410.853 | 0 | 2.9 | 5294 | 911.3 | 911.3 | 911.3 | 14.56 |
| 964 | K.QILGQLDGGSVAVLPVVDSTNQYLLDRIGELK.S | | 853.469 | 4 | 3410.856 | 3410.853 | 0 | 0.8 | 5294 | 847.0 | 847.0 | 847.0 | 13.32 |
| 964 | K.QILGQLDGGSVAVLPVVDSTNQYLLDRIGELK.S | | 682.977 | 5 | 3410.856 | 3410.853 | 0 | 0.8 | 5294 | 811.5 | 811.5 | 811.5 | 13.32 |
| 893 | K.QILGQLDGGSVAVLPVVDSTNQYLLDR.I | | 1435.766 | 2 | 2870.525 | 2870.526 | 0 | −0.4 | 5294 | 757.5 | 757.5 | 757.5 | 12.88 |
| 964 | K.QILGQLDGGSVAVLPVVDSTNQYLLDRIGELK.S | | 1137.623 | 3 | 3410.855 | 3410.853 | 0 | 0.6 | 5294 | 765.1 | 765.1 | 765.1 | 12.85 |
| 893 | K.QILGQLDGGSVAVLPVVDSTNQYLLDR.I | | 1435.768 | 2 | 2870.529 | 2870.526 | 0 | 0.9 | 5294 | 720.4 | 720.4 | 720.4 | 12.79 |
| 893 | K.QILGQLDGGSVAVLPVVDSTNQYLLDR.I | | 1435.769 | 2 | 2870.530 | 2870.526 | 0 | 1.4 | 5294 | 692.0 | 692.0 | 692.0 | 11.79 |
| 893 | K.QILGQLDGGSVAVLPVVDSTNQYLLDR.I | | 1435.771 | 2 | 2870.534 | 2870.526 | 0 | 2.8 | 5294 | 702.1 | 702.1 | 702.1 | 11.49 |
| 3127 | K.QILGQLDGGSVAVLPVVDSTN[+0.984]QYLLDRIGELK.S | N[+1] | 853.716 | 4 | 3411.841 | 3411.837 | 0 | 1.1 | 5294 | 711.6 | 711.6 | 310.7 | 11.32 |
| 964 | K.QILGQLDGGSVAVLPVVDSTNQYLLDRIGELK.S | | 853.470 | 4 | 3410.859 | 3410.853 | 0 | 1.9 | 5294 | 776.9 | 776.9 | 776.9 | 11.02 |
| 893 | K.QILGQLDGGSVAVLPVVDSTNQYLLDR.I | | 1435.769 | 2 | 2870.531 | 2870.526 | 0 | 1.7 | 5294 | 664.7 | 664.7 | 664.7 | 10.49 |
| 893 | K.QILGQLDGGSVAVLPVVDSTNQYLLDR.I | | 957.513 | 3 | 2870.525 | 2870.526 | 0 | −0.4 | 5294 | 664.6 | 664.6 | 664.6 | 10.34 |
| 893 | K.QILGQLDGGSVAVLPVVDSTNQYLLDR.I | | 957.514 | 3 | 2870.526 | 2870.526 | 0 | 0.1 | 5294 | 625.5 | 625.5 | 625.5 | 10.25 |
| 893 | K.QILGQLDGGSVAVLPVVDSTNQYLLDR.I | | 718.387 | 4 | 2870.526 | 2870.526 | 0 | 0.0 | 5294 | 603.2 | 603.2 | 603.2 | 10.25 |
| 1505 | K.QILGQLDGGSVAVLPVVDSTN[+0.984]QYLLDR.I | N[+1] | 1436.261 | 2 | 2871.515 | 2871.510 | 0 | 1.6 | 5294 | 607.0 | 607.0 | 85.3 | 9.49 |
| 893 | K.QILGQLDGGSVAVLPVVDSTNQYLLDR.I | | 957.513 | 3 | 2870.524 | 2870.526 | 0 | −0.6 | 5294 | 613.8 | 613.8 | 613.8 | 9.34 |
| 893 | K.QILGQLDGGSVAVLPVVDSTNQYLLDR.I | | 718.388 | 4 | 2870.529 | 2870.526 | 0 | 1.0 | 5294 | 576.8 | 576.8 | 576.8 | 9.25 |
| 893 | K.QILGQLDGGSVAVLPVVDSTNQYLLDR.I | | 1435.767 | 2 | 2870.526 | 2870.526 | 0 | 0.2 | 5294 | 559.7 | 559.7 | 559.7 | 9.12 |
| 893 | K.QILGQLDGGSVAVLPVVDSTNQYLLDR.I | | 957.514 | 3 | 2870.526 | 2870.526 | 0 | 0.0 | 5294 | 539.3 | 539.3 | 539.3 | 8.58 |
| 1505 | K.QILGQLDGGSVAVLPVVDSTN[+0.984]QYLLDR.I | N[+1] | 957.842 | 3 | 2871.512 | 2871.510 | 0 | 0.7 | 5294 | 529.6 | 529.6 | 235.0 | 8.58 |
| 893 | K.QILGQLDGGSVAVLPVVDSTNQYLLDR.I | | 718.387 | 4 | 2870.528 | 2870.526 | 0 | 0.7 | 5294 | 505.5 | 505.5 | 505.5 | 8.45 |
| 893 | K.QILGQLDGGSVAVLPVVDSTNQYLLDR.I | | 718.388 | 4 | 2870.530 | 2870.526 | 0 | 1.5 | 5294 | 499.7 | 499.7 | 499.7 | 8.45 |
| 893 | K.QILGQLDGGSVAVLPVVDSTNQYLLDR.I | | 957.515 | 3 | 2870.531 | 2870.526 | 0 | 1.7 | 5294 | 493.6 | 493.6 | 493.6 | 7.90 |
| 1505 | K.QILGQLDGGSVAVLPVVDSTN[+0.984]QYLLDR.I | N[+1] | 957.842 | 3 | 2871.512 | 2871.510 | 0 | 0.9 | 5294 | 550.8 | 550.8 | 110.9 | 7.81 |
| 1505 | K.QILGQLDGGSVAVLPVVDSTN[+0.984]QYLLDR.I | N[+1] | 957.842 | 3 | 2871.511 | 2871.510 | 0 | 0.5 | 5294 | 455.9 | 455.9 | 164.8 | 7.47 |
| 893 | K.QILGQLDGGSVAVLPVVDSTNQYLLDR.I | | 957.514 | 3 | 2870.526 | 2870.526 | 0 | 0.0 | 5294 | 368.5 | 368.5 | 368.5 | 7.42 |
| 893 | K.QILGQLDGGSVAVLPVVDSTNQYLLDR.I | | 957.516 | 3 | 2870.534 | 2870.526 | 0 | 2.8 | 5294 | 541.0 | 541.0 | 541.0 | 7.28 |
| 893 | K.QILGQLDGGSVAVLPVVDSTNQYLLDR.I | | 957.514 | 3 | 2870.527 | 2870.526 | 0 | 0.5 | 5294 | 371.1 | 371.1 | 371.1 | 6.99 |
| 3326 | K.QILGQLDGGSVAVLPVVDSTNQYLLDRIGELKSGDAC | C[+57] | 984.308 | 5 | 4917.512 | 4917.505 | 0 | 1.5 | 5294 | 406.9 | 406.9 | 406.9 | 5.45 |
| | [+57.021]IAEYQQAGR.G | | | | | | | | | | | | |
| 964 | K.QILGQLDGGSVAVLPVVDSTNQYLLDRIGELK.S | | 1137.614 | 3 | 3410.826 | 3410.853 | 0 | −7.9 | 5294 | 459.2 | 459.2 | 459.2 | 5.38 |
| 3326 | K.QILGQLDGGSVAVLPVVDSTNQYLLDRIGELKSGDAC | C[+57] | 984.310 | 5 | 4917.518 | 4917.505 | 0 | 2.7 | 5294 | 571.9 | 571.9 | 571.9 | 5.32 |
| | [+57.021]IAEYQQAGR.G | | | | | | | | | | | | |
| 893 | K.QILGQLDGGSVAVLPVVDSTNQYLLDR.I | | 957.513 | 3 | 2870.525 | 2870.526 | 0 | −0.4 | 5294 | 307.4 | 307.4 | 307.4 | 4.72 |
| 3326 | K.QILGQLDGGSVAVLPVVDSTNQYLLDRIGELKSGDAC | C[+57] | 984.309 | 5 | 4917.515 | 4917.505 | 0 | 1.9 | 5294 | 413.7 | 413.7 | 413.7 | 4.15 |
| | [+57.021]IAEYQQAGR.G | | | | | | | | | | | | |
| 893 | K.QILGQLDGGSVAVLPVVDSTNQYLLDR.I | | 957.856 | 3 | 2871.553 | 2870.526 | 1 | 8.1 | 5294 | 311.5 | 187.2 | 187.2 | 2.99 |
| 964 | K.QILGQLDGGSVAVLPVVDSTNQYLLDRIGELK.S | | 853.462 | 4 | 3410.824 | 3410.853 | 0 | −8.4 | 5294 | 322.6 | 322.6 | 322.6 | 2.82 |
| 1692 | R.IGELKSGDAC[+57.021]IAEYQQAGR.G | C[+57] | 689.337 | 3 | 2065.998 | 2065.997 | 0 | 0.2 | 5321 | 455.5 | 455.5 | 455.5 | 7.97 |
| 1698 | K.SGDAC[+57.021]IAEYQQAGR.G | C[+57] | 763.338 | 2 | 1525.670 | 1525.670 | 0 | −0.3 | 5326 | 596.6 | 596.6 | 596.6 | 9.58 |
| 1711 | K.SGDAC[+57.021]IAEYQQAGRGSR.G | C[+57] | 609.280 | 3 | 1825.825 | 1825.825 | 0 | 0.1 | 5326 | 537.5 | 537.5 | 537.5 | 8.31 |
| 1698 | K.SGDAC[+57.021]IAEYQQAGR.G | C[+57] | 509.228 | 3 | 1525.669 | 1525.670 | 0 | −0.4 | 5326 | 482.5 | 473.5 | 473.5 | 7.69 |
| 1698 | K.SGDAC[+57.021]IAEYQQAGR.G | C[+57] | 509.228 | 3 | 1525.669 | 1525.670 | 0 | −0.4 | 5326 | 338.1 | 338.1 | 338.1 | 5.32 |
| 4362 | K.RGPAAIGLGPVIGIVMAEALRK.L | | 548.330 | 4 | 2190.299 | 2189.295 | 1 | 0.2 | 5364 | 611.1 | 611.1 | 611.1 | 10.53 |
| 822 | K.RGPAAIGLGPVIGIVMAEALR.K | | 687.739 | 3 | 2061.201 | 2061.200 | 0 | 0.5 | 5364 | 519.7 | 519.7 | 519.7 | 8.98 |
| 2620 | R.GPAAIGLGPVIGIVMAEALR.K | | 953.054 | 2 | 1905.101 | 1905.099 | 0 | 1.2 | 5365 | 605.8 | 605.8 | 605.8 | 10.79 |
| 912 | R.VKWPNDLYLQDR.K | | 516.272 | 3 | 1546.801 | 1546.801 | 0 | 0.1 | 5393 | 535.5 | 462.3 | 462.3 | 8.10 |
| 912 | R.VKWPNDLYLQDR.K | | 516.272 | 3 | 1546.802 | 1546.801 | 0 | 0.2 | 5393 | 537.9 | 537.9 | 537.9 | 7.88 |
| 912 | R.VKWPNDLYLQDR.K | | 516.272 | 3 | 1546.801 | 1546.801 | 0 | 0.1 | 5393 | 467.5 | 419.2 | 419.2 | 7.76 |
| 919 | R.VKWPNDLYLQDRK.L | | 558.970 | 3 | 1674.896 | 1674.896 | 0 | −0.2 | 5393 | 495.4 | 495.4 | 495.4 | 7.63 |
| 912 | R.VKWPNDLYLQDR.K | | 773.905 | 2 | 1546.802 | 1546.801 | 0 | 0.5 | 5393 | 370.4 | 370.4 | 370.4 | 7.06 |
| 919 | R.VKWPNDLYLQDRK.L | | 558.970 | 3 | 1674.896 | 1674.896 | 0 | −0.2 | 5393 | 412.5 | 397.5 | 397.5 | 6.84 |
| 919 | R.VKWPNDLYLQDRK.L | | 419.480 | 4 | 1674.896 | 1674.896 | 0 | 0.0 | 5393 | 391.5 | 391.5 | 391.5 | 6.73 |
| 919 | R.VKWPNDLYLQDRK.L | | 558.970 | 3 | 1674.895 | 1674.896 | 0 | −0.5 | 5393 | 483.4 | 483.4 | 483.4 | 6.49 |
| 912 | R.VKWPNDLYLQDR.K | | 773.905 | 2 | 1546.802 | 1546.801 | 0 | 0.5 | 5393 | 320.1 | 320.1 | 320.1 | 5.78 |
| 2303 | K.WPNDLYLQDR.K | | 660.322 | 2 | 1319.637 | 1319.638 | 0 | 0.4 | 5395 | 418.8 | 270.4 | 270.4 | 5.97 |
| 857 | R.RVEESVVNQGWITLQEAGINLDRNTLAATLIR.E | | 716.791 | 5 | 3579.925 | 3579.924 | 0 | 0.1 | 5435 | 883.0 | 754.2 | 754.2 | 14.31 |
| 857 | R.RVEESVVNQGWITLQEAGINLDRNTLAATLIR.E | | 895.737 | 4 | 3579.926 | 3579.924 | 0 | 0.5 | 5435 | 846.8 | 846.8 | 846.8 | 13.85 |
| 857 | R.RVEESVVNQGWITLQEAGINLDRNTLAATLIR.E | | 895.737 | 4 | 3579.925 | 3579.924 | 0 | 0.3 | 5435 | 806.2 | 766.0 | 766.0 | 13.85 |
| 857 | R.RVEESVVNQGWITLQEAGINLDRNTLAATLIR.E | | 716.791 | 5 | 3579.926 | 3579.924 | 0 | 0.4 | 5435 | 801.3 | 659.9 | 659.9 | 13.32 |
| 857 | R.RVEESVVNQGWITLQEAGINLDRNTLAATLIR.E | | 895.737 | 4 | 3579.926 | 3579.924 | 0 | 0.4 | 5435 | 744.9 | 720.6 | 720.6 | 11.85 |
| 857 | R.RVEESVVNQGWITLQEAGINLDRNTLAATLIR.E | | 895.737 | 4 | 3579.927 | 3579.924 | 0 | 0.7 | 5435 | 716.9 | 625.8 | 625.8 | 11.85 |
| 1857 | R.RVEESVVNQGWITLQEAGINLDRNTLAATLIR.E | | 1193.980 | 3 | 3579.925 | 3579.924 | 0 | 0.3 | 5435 | 710.2 | 710.2 | 710.2 | 11.85 |
| 857 | R.RVEESVVNQGWITLQEAGINLDRNTLAATLIR.E | | 895.737 | 4 | 3579.926 | 3579.924 | 0 | 0.4 | 5435 | 669.4 | 669.4 | 669.4 | 10.85 |
| 1067 | R.RVEESVVNQGWITLQEAGINLDR.N | | 876.125 | 3 | 2626.360 | 2626.358 | 0 | 0.6 | 5435 | 621.6 | 621.6 | 621.6 | 10.24 |
| 857 | R.RVEESVVNQGWITLQEAGINLDRNTLAATLIR.E | | 895.737 | 4 | 3579.926 | 3579.924 | 0 | 0.4 | 5435 | 594.4 | 519.4 | 519.4 | 8.85 |
| 1067 | R.RVEESVVNQGWITLQEAGINLDR.N | | 876.125 | 3 | 2626.359 | 2626.358 | 0 | 0.3 | 5435 | 551.6 | 551.6 | 551.6 | 8.35 |
| 1292 | R.VEESVVNQGWITLQEAGINLDRNTLAATLIR.E | | 856.711 | 4 | 3423.822 | 3423.823 | 0 | −0.4 | 5436 | 828.5 | 828.5 | 828.5 | 13.32 |
| 1292 | R.VEESVVNQGWITLQEAGINLDRNTLAATLIR.E | | 1141.947 | 3 | 3423.826 | 3423.823 | 0 | 0.7 | 5436 | 701.7 | 701.7 | 701.7 | 11.85 |
| 1292 | R.VEESVVNQGWITLQEAGINLDRNTLAATLIR.E | | 857.213 | 4 | 3425.831 | 3423.823 | 2 | 0.4 | 5436 | 655.9 | 655.9 | 95.9 | 10.32 |
| 1292 | R.VEESVVNQGWITLQEAGINLDRNTLAATLIR.E | | 685.571 | 5 | 3423.827 | 3423.823 | 0 | 1.1 | 5436 | 594.4 | 550.5 | 550.5 | 8.32 |
| 4657 | R.VEES[+79.966]VVN[+0.984]QGWITLQEAGINLDRNT | N[+1], | 1328.994 | 3 | 3984.967 | 3982.968 | 2 | −1.9 | 5436 | 312.2 | 312.2 | 1.0 | 3.20 |
| | [+79.966]LAATLIRELR.A | S[+80], | | | | | | | | | | | |
| | | T[+80] | | | | | | | | | | | |
| 1032 | R.AALELFEQEGLAPYLPRWEK.L | | 787.415 | 3 | 2360.231 | 2360.229 | 0 | 0.9 | 5470 | 719.5 | 719.5 | 719.5 | 12.53 |
| 1032 | R.AALELFEQEGLAPYLPRWEK.L | | 787.416 | 3 | 2360.232 | 2360.229 | 0 | 1.6 | 5470 | 643.9 | 643.9 | 643.9 | 10.53 |
| 1032 | R.AALELFEQEGLAPYLPRWEK.L | | 590.813 | 4 | 2360.229 | 2360.229 | 0 | 0.0 | 5470 | 576.4 | 576.4 | 576.4 | 8.99 |
| 1914 | R.AALELFEQEGLAPYLPR.W | | 959.010 | 2 | 1917.012 | 1917.012 | 0 | 0.4 | 5470 | 452.6 | 452.6 | 452.6 | 8.78 |
| 1914 | R.AALELFEQEGLAPYLPR.W | | 959.011 | 2 | 1917.015 | 1917.012 | 0 | 2.0 | 5470 | 455.8 | 455.8 | 455.8 | 6.93 |
| 1032 | R.AALELFEQEGLAPYLPRWEK.L | | 1181.120 | 2 | 2361.233 | 2360.229 | 1 | 0.4 | 5470 | 368.8 | 304.8 | 304.8 | 5.92 |
| 973 | R.WEKLDNFINRPVK.L | | 553.639 | 3 | 1658.901 | 1658.901 | 0 | 0.1 | 5487 | 540.5 | 540.5 | 540.5 | 8.10 |
| 973 | R.WEKLDNFINRPVK.L | | 415.481 | 4 | 1658.901 | 1658.901 | 0 | −0.2 | 5487 | 400.2 | 400.2 | 400.2 | 7.18 |
| 973 | R.WEKLDNFINRPVK.L | | 829.954 | 2 | 1658.901 | 1658.901 | 0 | −0.1 | 5487 | 332.6 | 332.6 | 332.6 | 4.25 |
| 820 | K.LDNFINRPVKLIIGDKEIFGISR.G | | 665.134 | 4 | 2657.516 | 2657.514 | 0 | 0.8 | 5490 | 465.4 | 465.4 | 465.4 | 7.63 |
| 820 | K.LDNFINRPVKLIIGDKEIFGISR.G | | 532.309 | 5 | 2657.514 | 2657.514 | 0 | 0.1 | 5490 | 444.6 | 444.6 | 444.6 | 7.63 |
| 1931 | K.LDNFINRPVK.L | | 608.346 | 2 | 1215.684 | 1215.684 | 0 | 0.0 | 5490 | 459.0 | 459.0 | 459.0 | 7.60 |
| 820 | K.LDNFINRPVKLIIGDKEIFGISR.G | | 665.134 | 4 | 2657.515 | 2657.514 | 0 | 0.5 | 5490 | 472.0 | 472.0 | 472.0 | 7.41 |
| 1931 | K.LDNFINRPVK.L | | 405.900 | 3 | 1215.684 | 1215.684 | 0 | 0.0 | 5490 | 397.2 | 397.2 | 397.2 | 7.14 |
| 1931 | K.LDNFINRPVK.L | | 405.900 | 3 | 1215.684 | 1215.684 | 0 | −0.1 | 5490 | 347.7 | 347.7 | 347.7 | 6.04 |
| 1931 | K.LDNFINRPVK.L | | 405.900 | 3 | 1215.684 | 1215.684 | 0 | −0.1 | 5490 | 335.1 | 335.1 | 335.1 | 5.86 |
| 820 | K.LDNFINRPVKLIIGDKEIFGISR.G | | 665.134 | 4 | 2657.513 | 2657.514 | 0 | 0.3 | 5490 | 305.6 | 305.6 | 305.6 | 4.92 |
| 983 | K.LIIGDKEIFGISR.G | | 487.621 | 3 | 1460.847 | 1460.847 | 0 | −0.2 | 5500 | 660.5 | 660.5 | 660.5 | 11.32 |
| 983 | K.LIIGDKEIFGISR.G | | 487.621 | 3 | 1460.848 | 1460.847 | 0 | 0.3 | 5500 | 597.8 | 597.8 | 597.8 | 9.32 |
| 983 | K.LIIGDKEIFGISR.G | | 487.621 | 3 | 1460.847 | 1460.847 | 0 | −0.1 | 5500 | 562.7 | 562.7 | 562.7 | 9.32 |
| 983 | K.LIIGDKEIFGISR.G | | 730.927 | 2 | 1460.847 | 1460.847 | 0 | 0.1 | 5500 | 554.2 | 554.2 | 554.2 | 8.65 |
| 983 | K.LIIGDKEIFGISR.G | | 487.621 | 3 | 1460.847 | 1460.847 | 0 | 0.0 | 5500 | 397.0 | 397.0 | 397.0 | 7.27 |
| 983 | K.LIIGDKEIFGISR.G | | 487.955 | 3 | 1461.850 | 1460.847 | 1 | −0.2 | 5500 | 344.2 | 344.2 | 344.2 | 4.00 |
| 1612 | R.GIDKQGALLLEQDGVIKPWM[+15.995]GGEISLR.S | M[+16] | 980.527 | 3 | 2939.568 | 2939.566 | 0 | 0.6 | 5513 | 825.1 | 825.1 | 825.1 | 14.54 |
| 837 | R.GIDKQGALLLEQDGVIKPWMGGEISLR.S | | 975.196 | 3 | 2923.573 | 2923.571 | 0 | 0.6 | 5513 | 819.7 | 819.7 | 819.7 | 14.54 |
| 837 | R.GIDKQGALLLEQDGVIKPWMGGEISLR.S | | 975.196 | 3 | 2923.573 | 2923.571 | 0 | 0.6 | 5513 | 798.6 | 798.6 | 798.6 | 13.53 |
| 837 | R.GIDKQGALLLEQDGVIKPWMGGEISLR.S | | 731.648 | 4 | 2923.572 | 2923.571 | 0 | 0.2 | 5513 | 767.7 | 767.7 | 767.7 | 13.53 |
| 837 | R.GIDKQGALLLEQDGVIKPWMGGEISLR.S | | 731.649 | 4 | 2923.573 | 2923.571 | 0 | 0.5 | 5513 | 755.5 | 755.5 | 755.5 | 13.53 |
| 1837 | R.GIDKQGALLLEQDGVIKPWMGGEISLR.S | | 975.195 | 3 | 2923.570 | 2923.571 | 0 | −0.3 | 5513 | 733.7 | 733.7 | 733.7 | 12.53 |
| 1612 | R.GIDKQGALLLEQDGVIKPWM[+15.995]GGEISLR.S | M[+16] | 980.528 | 3 | 2939.569 | 2939.566 | 0 | 1.1 | 5513 | 719.2 | 719.2 | 719.2 | 12.53 |
| 837 | R.GIDKQGALLLEQDGVIKPWMGGEISLR.S | | 731.648 | 4 | 2923.570 | 2923.571 | 0 | −0.2 | 5513 | 706.1 | 706.1 | 706.1 | 12.53 |
| 837 | R.GIDKQGALLLEQDGVIKPWMGGEISLR.S | | 731.648 | 4 | 2923.570 | 2923.571 | 0 | −0.2 | 5513 | 681.9 | 681.9 | 681.9 | 11.53 |
| 837 | R.GIDKQGALLLEQDGVIKPWMGGEISLR.S | | 731.649 | 4 | 2923.573 | 2923.571 | 0 | 0.5 | 5513 | 674.7 | 674.7 | 674.7 | 11.53 |
| 1837 | R.GIDKQGALLLEQDGVIKPWMGGEISLR.S | | 731.649 | 4 | 2923.572 | 2923.571 | 0 | 0.4 | 5513 | 674.2 | 674.2 | 674.2 | 11.53 |
| 837 | R.GIDKQGALLLEQDGVIKPWMGGEISLR.S | | 975.195 | 3 | 2923.571 | 2923.571 | 0 | 0.0 | 5513 | 671.2 | 671.2 | 671.2 | 11.53 |
| 837 | R.GIDKQGALLLEQDGVIKPWMGGEISLR.S | | 731.649 | 4 | 2923.573 | 2923.571 | 0 | 0.6 | 5513 | 669.7 | 669.7 | 669.7 | 11.53 |
| 837 | R.GIDKQGALLLEQDGVIKPWMGGEISLR.S | | 585.520 | 5 | 2923.571 | 2923.571 | 0 | −0.1 | 5513 | 694.1 | 694.1 | 694.1 | 10.99 |
| 837 | R.GIDKQGALLLEQDGVIKPWMGGEISLR.S | | 731.649 | 4 | 2923.572 | 2923.571 | 0 | 0.3 | 5513 | 641.0 | 641.0 | 641.0 | 10.53 |
| 1612 | R.GIDKQGALLLEQDGVIKPWM[+15.995]GGEISLR.S | M[+16] | 735.648 | 4 | 2939.569 | 2939.566 | 0 | 1.1 | 5513 | 626.1 | 626.1 | 626.1 | 10.53 |
| 837 | R.GIDKQGALLLEQDGVIKPWMGGEISLR.S | | 731.649 | 4 | 2923.573 | 2923.571 | 0 | 0.6 | 5513 | 623.6 | 623.6 | 623.6 | 10.53 |
| 837 | R.GIDKQGALLLEQDGVIKPWMGGEISLR.S | | 975.195 | 3 | 2923.569 | 2923.571 | 0 | −0.6 | 5513 | 629.8 | 629.8 | 629.8 | 9.61 |
| 837 | R.GIDKQGALLLEQDGVIKPWMGGEISLR.S | | 731.649 | 4 | 2923.572 | 2923.571 | 0 | 0.4 | 5513 | 590.0 | 590.0 | 590.0 | 9.53 |
| 1837 | R.GIDKQGALLLEQDGVIKPWMGGEISLR.S | | 731.648 | 4 | 2923.571 | 2923.571 | 0 | 0.1 | 5513 | 583.7 | 583.7 | 583.7 | 9.53 |
| 837 | R.GIDKQGALLLEQDGVIKPWMGGEISLR.S | | 731.648 | 4 | 2923.572 | 2923.571 | 0 | 0.3 | 5513 | 577.2 | 577.2 | 577.2 | 9.53 |
| 1612 | R.GIDKQGALLLEQDGVIKPWM[+15.995]GGEISLR.S | M[+16] | 735.648 | 4 | 2939.569 | 2939.566 | 0 | 1.0 | 5513 | 576.9 | 576.9 | 576.9 | 9.53 |
| 837 | R.GIDKQGALLLEQDGVIKPWMGGEISLR.S | | 731.649 | 4 | 2923.572 | 2923.571 | 0 | 0.3 | 5513 | 569.1 | 569.1 | 569.1 | 8.98 |
| 1612 | R.GIDKQGALLLEQDGVIKPWM[+15.995]GGEISLR.S | M[+16] | 735.898 | 4 | 2940.570 | 2939.566 | 1 | 0.2 | 5513 | 537.3 | 537.3 | 537.3 | 8.86 |
| 837 | R.GIDKQGALLLEQDGVIKPWMGGEISLR.S | | 731.649 | 4 | 2923.574 | 2923.571 | 0 | 1.0 | 5513 | 532.6 | 532.6 | 532.6 | 8.86 |
| 837 | R.GIDKQGALLLEQDGVIKPWMGGEISLR.S | | 731.648 | 4 | 2923.572 | 2923.571 | 0 | 0.3 | 5513 | 525.4 | 525.4 | 525.4 | 8.86 |
| 837 | R.GIDKQGALLLEQDGVIKPWMGGEISLR.S | | 731.648 | 4 | 2923.572 | 2923.571 | 0 | 0.3 | 5513 | 499.4 | 499.4 | 499.4 | 8.72 |
| 1612 | R.GIDKQGALLLEQDGVIKPWM[+15.995]GGEISLR.S | M[+16] | 735.647 | 4 | 2939.568 | 2939.566 | 0 | 0.6 | 5513 | 1494.4 | 494.4 | 494.4 | 8.72 |
| 1612 | R.GIDKQGALLLEQDGVIKPWM[+15.995]GGEISLR.S | M[+16] | 980.527 | 3 | 2939.567 | 2939.566 | 0 | 0.4 | 5513 | 530.5 | 530.5 | 530.5 | 8.31 |
| 837 | R.GIDKQGALLLEQDGVIKPWMGGEISLR.S | | 975.195 | 3 | 2923.571 | 2923.571 | 0 | 0.0 | 5513 | 491.8 | 491.8 | 491.8 | 7.75 |
| 837 | R.GIDKQGALLLEQDGVIKPWMGGEISLR.S | | 731.648 | 4 | 2923.572 | 2923.571 | 0 | 0.3 | 5513 | 399.6 | 399.6 | 399.6 | 7.70 |
| 1612 | R.GIDKQGALLLEQDGVIKPWM[+15.995]GGEISLR.S | M[+16] | 980.528 | 3 | 2939.569 | 2939.566 | 0 | 1.1 | 5513 | 462.1 | 462.1 | 462.1 | 7.55 |
| 837 | R.GIDKQGALLLEQDGVIKPWMGGEISLR.S | | 731.648 | 4 | 2923.572 | 2923.571 | 0 | 0.3 | 5513 | 390.2 | 390.2 | 390.2 | 7.47 |
| 1612 | R.GIDKQGALLLEQDGVIKPWM[+15.995]GGEISLR.S | M[+16] | 981.195 | 3 | 2941.572 | 2939.566 | 2 | −0.3 | 5513 | 413.7 | 413.7 | 413.7 | 7.39 |
| 837 | R.GIDKQGALLLEQDGVIKPWMGGEISLR.S | | 731.649 | 4 | 2923.573 | 2923.571 | 0 | 0.6 | 5513 | 401.5 | 401.5 | 401.5 | 7.39 |
| 837 | R.GIDKQGALLLEQDGVIKPWMGGEISLR.S | | 731.649 | 4 | 2923.572 | 2923.571 | 0 | 0.4 | 5513 | 348.0 | 348.0 | 348.0 | 5.99 |
| 837 | R.GIDKQGALLLEQDGVIKPWMGGEISLR.S | | 731.900 | 4 | 2924.579 | 2923.571 | 1 | 1.7 | 5513 | 391.8 | 391.8 | 391.8 | 5.79 |
| 837 | R.GIDKQGALLLEQDGVIKPWMGGEISLR.S | | 731.648 | 4 | 2923.571 | 2923.571 | 0 | 0.1 | 5513 | 307.9 | 307.9 | 307.9 | 5.71 |
| 837 | R.GIDKQGALLLEQDGVIKPWMGGEISLR.S | | 731.649 | 4 | 2923.575 | 2923.571 | 0 | 1.3 | 5513 | 312.4 | 312.4 | 312.4 | 5.53 |
| 837 | R.GIDKQGALLLEQDGVIKPWMGGEISLR.S | | 731.649 | 4 | 2923.573 | 2923.571 | 0 | 0.7 | 5513 | 304.0 | 304.0 | 304.0 | 5.53 |
| 837 | R.GIDKQGALLLEQDGVIKPWMGGEISLR.S | | 585.520 | 5 | 2923.569 | 2923.571 | 0 | −0.8 | 5513 | 320.0 | 320.0 | 320.0 | 4.35 |
| 915 | K.QGALLLEQDGVIKPWMGGEISLR.S | | 628.341 | 4 | 2510.343 | 2510.344 | 0 | −0.1 | 5517 | 666.9 | 666.9 | 666.9 | 11.25 |
| 915 | K.QGALLLEQDGVIKPWMGGEISLR.S | | 837.453 | 3 | 2510.344 | 2510.344 | 0 | 0.2 | 5517 | 600.7 | 600.7 | 600.7 | 10.79 |
| 1659 | K.QGALLLEQDGVIKPWM[+15.995]GGEISLR.S | M[+16] | 842.784 | 3 | 2526.337 | 2526.339 | 0 | −0.5 | 5517 | 643.7 | 643.7 | 643.7 | 9.87 |
| 915 | K.QGALLLEQDGVIKPWMGGEISLR.S | | 1255.675 | 2 | 2510.342 | 2510.344 | 0 | −0.6 | 5517 | 643.1 | 643.1 | 643.1 | 9.87 |
| 1659 | K.QGALLLEQDGVIKPWM[+15.995]GGEISLR.S | M[+16] | 842.783 | 3 | 2526.335 | 2526.339 | 0 | −1.3 | 5517 | 577.7 | 577.7 | 577.7 | 8.33 |
| 864 | R.SAEKLQLPPLER.L | | 690.896 | 2 | 1380.784 | 1380.785 | 0 | −0.1 | 5540 | 646.0 | 646.0 | 646.0 | 10.32 |
| 864 | R.SAEKLQLPPLER.L | | 690.896 | 2 | 1380.784 | 1380.785 | 0 | −0.2 | 5540 | 572.9 | 572.9 | 572.9 | 9.32 |
| 864 | R.SAEKLQLPPLER.L | | 690.896 | 2 | 1380.784 | 1380.785 | 0 | −0.2 | 5540 | 561.2 | 561.2 | 561.2 | 8.77 |
| 864 | R.SAEKLQLPPLER.L | | 690.896 | 2 | 1380.784 | 1380.785 | 0 | −0.1 | 5540 | 555.2 | 555.2 | 555.2 | 8.65 |
| 864 | R.SAEKLQLPPLER.L | | 460.933 | 3 | 1380.785 | 1380.785 | 0 | 0.1 | 5540 | 441.7 | 441.7 | 441.7 | 7.76 |
| 864 | R.SAEKLQLPPLER.L | | 460.933 | 3 | 1380.784 | 1380.785 | 0 | −0.2 | 5540 | 480.0 | 480.0 | 480.0 | 7.54 |
| 864 | R.SAEKLQLPPLER.L | | 690.896 | 2 | 1380.784 | 1380.785 | 0 | −0.1 | 5540 | 399.9 | 399.9 | 399.9 | 7.27 |
| 4650 | R.SAEKLQLPPLERLTLD.- | | 912.017 | 2 | 1823.027 | 1823.027 | 0 | 0.1 | 5540 | 431.4 | 431.4 | 431.4 | 7.26 |
| 864 | R.SAEKLQLPPLER.L | | 460.933 | 3 | 1380.784 | 1380.785 | 0 | −0.7 | 5540 | 531.2 | 531.2 | 531.2 | 7.19 |
| 864 | R.SAEKLQLPPLER.L | | 460.933 | 3 | 1380.785 | 1380.785 | 0 | 0.0 | 5540 | 364.7 | 364.7 | 364.7 | 7.06 |
| 864 | R.SAEKLQLPPLER.L | | 460.933 | 3 | 1380.785 | 1380.785 | 0 | 0.0 | 5540 | 335.5 | 335.5 | 335.5 | 5.78 |
| 864 | R.SAEKLQLPPLER.L | | 460.933 | 3 | 1380.784 | 1380.785 | 0 | −0.3 | 5540 | 329.0 | 329.0 | 329.0 | 5.78 |
| 1905 | K.LQLPPLER.L | | 483.292 | 2 | 965.578 | 965.578 | 0 | −0.3 | 5544 | 429.6 | 429.6 | 429.6 | 7.44 |
| 1905 | K.LQLPPLER.L | | 483.293 | 2 | 965.578 | 965.578 | 0 | −0.2 | 5544 | 371.2 | 371.2 | 371.2 | 7.32 |
| 1905 | K.LQLPPLER.L | | 483.293 | 2 | 965.578 | 965.578 | 0 | 0.0 | 5544 | 370.4 | 370.4 | 370.4 | 7.14 |
| 1905 | K.LQLPPLER.L | | 483.293 | 2 | 965.578 | 965.578 | 0 | 0.1 | 5544 | 361.1 | 361.1 | 361.1 | 7.14 |
| 4651 | K.LQLPPLERLTLD.- | | 704.414 | 2 | 1407.820 | 1407.821 | 0 | −0.2 | 5544 | 346.6 | 346.6 | 346.6 | 5.98 |
| 1905 | K.LQLPPLER.L | | 483.293 | 2 | 965.578 | 965.578 | 0 | 0.3 | 5544 | 319.3 | 319.3 | 319.3 | 5.58 |

TABLE 10
Unique in N-Turbo vs. Control and C-Turbo vs. Control

| | | Avg | Avg | Avg | Avg % | Avg % |
|---|---|---|---|---|---|---|
| | | PSMs | PSMs | PSMs in | Sequence | Sequence |
| | | in C- | in N- | Control | Coverage | Coverage |
| Accession | Description | Turbo | Turbo | Turbo | C-Turbo | N-Turbo |
| Q14119 | Vascular endothelial zinc finger 1 OS = Homo sapiens OX = 9606 GN = VEZF1 PE = 1 SV = 2-[VEZF1_HUMAN] | 0 | 4 | 0 | 0.0 | 4.9 |
| Q16850-2 | Isoform 2 of Lanosterol 14-alpha demethylase OS = Homo sapiens OX=9606 GN=CYP51A1 - [CP51A_HUMAN] | 3 | 4 | 0 | 5.9 | 7.8 |
| O96013 | Serine/threonine-protein kinase PAK 4 OS = Homo sapiens OX = 9606 GN = PAK4 PE = 1 | 1 | 4 | 0 | 1.6 | 4.7 |
| | SV = 1 - [PAK4_HUMAN] | | | | | |
| O15269 | Serine palmitoyltransferase 1 OS = Homo sapiens OX = 9606 GN = SPTLC1 PE = 1 SV = 1 - [SPTC1_HUMAN] | 3 | 3 | 0 | 6.7 | 7.8 |
| Q08AE8-2 | Isoform 2 of Protein spire homolog 1 OS = Homo sapiens OX = 9606 GN = SPIRE1 - [SPIR1_HUMAN] | 3 | 3 | 0 | 3.0 | 3.0 |
| Q86SQ0-2 | Isoform 2 of Pleckstrin homology-like domain family B member 2 OS = Homo sapiens OX = 9606 GN = | 2 | 3 | 0 | 1.5 | 3.1 |
| | PHLDB2 - [PHLB2_HUMAN] | | | | | |
| Q969X6-2 | Isoform 2 of U3 small nucleolar RNA-associated protein 4 homolog OS=Homo sapiens OX = 9606 | 0 | 3 | 0 | 0.0 | 8.1 |
| | GN = UTP4 - [UTP4_HUMAN] | | | | | |
| Q02750-2 | Isoform 2 of Dual specificity mitogen-activated protein kinase kinase 1 OS = Homo sapiens | 2 | 3 | 0 | 6.3 | 14.7 |
| | OX = 9606 GN = MAP2K1 - [MP2K1_HUMAN] | | | | | |
| ASYKK6 | CCR4-NOT transcription complex subunit 1 OS = Homo sapiens OX = 9606 GN = CNOT1 PE = 1 | 1 | 3 | 0 | 0.5 | 1.9 |
| | SV = 2 - [CNOT1_HUMAN] | | | | | |
| Q15773 | Myeloid leukemia factor 2 OS = Homo sapiens OX = 9606 GN = MLF2 PE = 1 SV = 1 - [MLF2_HUMAN] | 0 | 3 | 0 | 0.0 | 15.7 |
| Q99755-3 | Isoform 3 of Phosphatidylinositol 4-phosphate 5-kinase type-1 alpha OS = Homo sapiens OX = | 0 | 3 | 0 | 0.0 | 8.6 |
| | 9606 GN = PIP5K1A - [PI51A_HUMAN] | | | | | |
| Q9NQC7-2 | Isoform 2 of Ubiquitin carboxyl-terminal hydrolase CYLD OS = Homo sapiens OX = 9606 GN = | 8 | 2 | 0 | 9.2 | 3.1 |
| | CYLD - [CYLD_HUMAN] | | | | | |
| Q13045-2 | Isoform 2 of Protein flightless-1 homolog OS = Homo sapiens OX = 9606 GN = FLII - [FLII_HUMAN] | 4 | 2 | 0 | 3.6 | 2.2 |
| Q96CB8 | Integrator complex subunit 12 OS = Homo sapiens OX = 9606 GN = INTS12 PE = 1 SV = 1 - [INT12_HUMAN] | 1 | 2 | 0 | 1.8 | 5.2 |
| P22061 | Protein-L-isoaspartate(D-aspartate) O-methyltransferase OS = Homo sapiens OX = 9606 GN = | 2 | 2 | 0 | 16.6 | 8.7 |
| | PCMT1 PE = 1 SV = 4 - [PIMT_HUMAN] | | | | | |
| O75477 | Erlin-1 OS = Homo sapiens OX = 9606 GN = ERLIN1 PE = 1 SV = 2 - [ERLN1_HUMAN] | 2 | 2 | 0 | 6.3 | 9.5 |
| Q15154-4 | Isoform 4 of Pericentriolar material 1 protein OS = Homo sapiens OX = 9606 GN = PCM1 - [PCM1_HUMAN] | 1 | 2 | 0 | 0.3 | 1.3 |
| P30533 | Alpha-2-macroglobulin receptor-associated protein OS = Homo sapiens OX = 9606 GN = LRPAP1 PE = | 0 | 2 | 0 | 0.0 | 8.3 |
| | 1 SV = 1 - [AMRP_HUMAN] | | | | | |
| Q6P1L5 | Protein FAM117B OS = Homo sapiens OX = 9606 GN = FAM117B PE = 1 SV = 2 - [F117B_HUMAN] | 0 | 2 | 0 | 0.0 | 3.2 |
| Q6XZF7 | Dynamin-binding protein OS = Homo sapiens OX = 9606 GN = DNMBP PE = 1 SV = 1 - [DNMBP_HUMAN] | 0 | 2 | 0 | 0.0 | 1.5 |
| Q7Z5Q1-7 | Isoform 6 of Cytoplasmic polyadenylation element-binding protein 2 OS = Homo sapiens OX = 9606 | 0 | 2 | 0 | 0.0 | 4.0 |
| | GN = CPEB2 - [CPEB2_HUMAN] | | | | | |
| Q9UHD2 | Serine/threonine-protein kinase TBK1 OS = Homo sapiens OX = 9606 GN = TBK1 PE = 1 SV = | 2 | 2 | 0 | 5.5 | 2.8 |
| | 1 - [TBK1_HUMAN] | | | | | |
| A6NED2 | RCC1 domain-containing protein 1 OS = Homo sapiens OX = 9606 GN = RCCD1 PE = 1 SV = | 2 | 2 | 0 | 7.6 | 9.3 |
| | 1 - [RCCD1_HUMAN] | | | | | |
| Q99456 | Keratin, type I cytoskeletal 12 OS = Homo sapiens OX = 9606 GN = KRT12 PE = 1 SV = | 1 | 2 | 0 | 1.2 | 1.8 |
| | 1 - [K1C12_HUMAN] | | | | | |
| O60716-21 | Isoform 3A of Catenin delta-1 OS = Homo sapiens OX = 9606 GN = CTNND1 - [CTND1_HUMAN] | 1 | 2 | 0 | 2.0 | 3.6 |
| Q8NB46 | Serine/threonine-protein phosphatase 6 regulatory ankyrin repeat subunit C OS = Homo sapiens | 1 | 2 | 0 | 1.5 | 2.2 |
| | OX = 9606 GN = ANKRD52 PE = 1 SV = 3 - [ANR52_HUMAN] | | | | | |
| P06789 | Replication protein E1 OS = Human papillomavirus type 18 OX = 333761 GN = E1 PE = 1 SV = | 1 | 2 | 0 | 1.6 | 2.2 |
| | 1 - [VE1_HPV18] | | | | | |
| P62857 | 40S ribosomal protein S28 OS = Homo sapiens OX = 9606 GN = RPS28 PE = 1 SV = 1 - [RS28_HUMAN] | 1 | 2 | 0 | 7.7 | 24.2 |
| P31751-2 | Isoform 2 of RAC-beta serine/threonine-protein kinase OS = Homo sapiens OX = 9606 GN = | 0 | 2 | 0 | 0.0 | 3.5 |
| | AKT2 - [AKT2_HUMAN] | | | | | |
| P82650 | 28S ribosomal protein S22, mitochondrial OS = Homo sapiens OX = 9606 GN = MRPS22 PE = 1 SV = | 0 | 2 | 0 | 0.0 | 8.0 |
| | 1 - [RT22_HUMAN] | | | | | |
| Q13131 | 5′-AMP-activated protein kinase catalytic subunit alpha-1 OS = Homo sapiens OX = 9606 GN = | 0 | 2 | 0 | 0.0 | 3.5 |
| | PRKAA1 PE = 1 SV = 4 - [AAPK1_HUMAN] | | | | | |
| Q92616 | elF-2-alpha kinase activator GCN1 OS = Homo sapiens OX = 9606 GN = GCN1 PE = 1 SV = | 0 | 2 | 0 | 0.0 | 0.9 |
| | 6 - [GCN1_HUMAN] | | | | | |
| Q92625 | Ankyrin repeat and SAM domain-containing protein 1A OS = Homo sapiens OX = 9606 GN = | 0 | 2 | 0 | 0.0 | 2.7 |
| | ANKS1A PE = 1 SV = 4 - [ANS1A_HUMAN] | | | | | |
| Q9HD45 | Transmembrane 9 superfamily member 3 OS = Homo sapiens OX = 9606 GN = TM9SF3 PE = 1 SV = | 0 | 2 | 0 | 0.0 | 2.6 |
| | 2 - [TM9S3_HUMAN] | | | | | |
| Q9UGR2 | Zinc finger CCCH domain-containing protein 7B OS = Homo sapiens OX = 9606 GN = ZC3H7B PE = | 0 | 2 | 0 | 0.0 | 1.2 |
| | 1 SV = 2 - [Z3H7B_HUMAN] | | | | | |
| Q9H0W8-2 | Isoform 2 of Protein SMG9 OS = Homo sapiens OX = 9606 GN = SMG9 - [SMG9_HUMAN] | 1 | 1 | 0 | 2.3 | 5.7 |
| O14925 | Mitochondrial import inner membrane translocase subunit Tim23 OS = Homo sapiens OX = | 1 | 1 | 0 | 4.5 | 8.6 |
| | 9606 GN = TIMM23 PE = 1 SV = 1 - [TIM23_HUMAN] | | | | | |
| P02662 | CRAP CASA1_BOVIN Alpha-S1-casein OS = Bos taurus GN = CSN1S1 PE = 1 SV = 2 | 1 | 1 | 0 | 3.4 | 6.9 |
| P08574 | Cytochrome c1, heme protein, mitochondrial OS = Homo sapiens OX = 9606 GN = CYC1 PE = | 1 | 1 | 0 | 2.9 | 5.7 |
| | 1 SV = 3 - [CY1_HUMAN] | | | | | |
| P16989-2 | Isoform 2 of Y-box-binding protein 3 OS = Homo sapiens OX = 9606 GN = YBX3 - [YBOX3_HUMAN] | 1 | 1 | 0 | 5.3 | 5.3 |
| P45973 | Chromobox protein homolog 5 OS = Homo sapiens OX = 9606 GN = CBX5 PE = 1 SV = 1 - [CBX5_HUMAN] | 1 | 1 | 0 | 3.3 | 6.8 |
| P78312-4 | Isoform 4 of Protein FAM193A OS = Homo sapiens OX = 9606 GN = FAM193A - [F193A_HUMAN] | 1 | 1 | 0 | 1.0 | 2.7 |
| Q7Z4G4-3 | Isoform 3 of tRNA (guanine(10)-N2)-methyltransferase homolog OS = Homo sapiens OX = 9606 | 1 | 1 | 0 | 1.7 | 4.8 |
| | GN = TRMT11 - [TRM11_HUMAN] | | | | | |
| O60493-4 | Isoform 4 of Sorting nexin-3 OS = Homo sapiens OX = 9606 GN = SNX3 - [SNX3_HUMAN] | 0 | 1 | 0 | 0.0 | 13.1 |
| O75128 | Protein cordon-bleu OS = Homo sapiens OX = 9606 GN = COBL PE = 1 SV = 2 - [COBL_HUMAN] | 0 | 1 | 0 | 0.0 | 1.5 |
| P14373-2 | Isoform Beta of Zinc finger protein RFP OS = Homo sapiens OX = 9606 GN = | 0 | 1 | 0 | 0.0 | 6.0 |
| | TRIM27 - [TRI27_HUMAN] | | | | | |
| P14635-2 | Isoform 2 of G2/mitotic-specific cyclin-B1 OS = Homo sapiens OX = 9606 GN = | 0 | 1 | 0 | 0.0 | 4.7 |
| | CCNB1 - [CCNB1_HUMAN] | | | | | |
| Q15155 | Nodal modulator 1 OS = Homo sapiens OX = 9606 GN = NOMO1 PE = 1 SV = 5 - [NOMO1_HUMAN] | 0 | 1 | 0 | 0.0 | 1.7 |
| Q6SJ93-2 | Isoform 2 of Protein FAM111B OS = Homo sapiens OX = 9606 GN = FAM111B - [F111B_HUMAN] | 0 | 1 | 0 | 0.0 | 1.9 |
| Q7Z739 | YTH domain-containing family protein 3 OS = Homo sapiens OX = 9606 GN = YTHDF3 PE = | 0 | 1 | 0 | 0.0 | 2.2 |
| | 1 SV = 1 - [YTHD3_HUMAN] | | | | | |
| Q8NI08-2 | Isoform 2 of Nuclear receptor coactivator 7 OS = Homo sapiens OX = 9606 GN = | 0 | 1 | 0 | 0.0 | 0.9 |
| | NCOA7 - [NCOA7_HUMAN] | | | | | |
| Q99755-4 | Isoform 4 of Phosphatidylinositol 4-phosphate 5-kinase type-1 alpha OS = Homo sapiens | 0 | 1 | 0 | 0.0 | 4.4 |
| | OX = 9606 GN = PIP5K1A - [PI51A_HUMAN] | | | | | |
| Q9BSH4 | Translational activator of cytochrome c oxidase 1 OS = Homo sapiens OX = 9606 GN = | 0 | 1 | 0 | 0.0 | 7.6 |
| | TACO1 PE = 1 SV = 1 - [TACO1_HUMAN] | | | | | |
| Q9NX74 | tRNA-dihydrouridine(20) synthase [NAD(P)+]-like OS = Homo sapiens OX = 9606 GN = DUS2 | 0 | 1 | 0 | 0.0 | 3.0 |
| | PE = 1 SV = 1 - [DUS2L_HUMAN] | | | | | |
| Q9UI30 | Multifunctional methyltransferase subunit TRM112-like protein OS = Homo sapiens | 0 | 1 | 0 | 0.0 | 14.9 |
| | OX = 9606 GN = TRMT112 PE = 1 SV = 1 - [TR112_HUMAN] | | | | | |
| Q9UN86-2 | Isoform B of Ras GTPase-activating protein-binding protein 2 OS = Homo sapiens OX = | 0 | 1 | 0 | 0.0 | 6.1 |
| | 9606 GN = G3BP2 - [G3BP2_HUMAN] | | | | | |
| Q9Y232-3 | Isoform 3 of Chromodomain Y-like protein OS = Homo sapiens OX = 9606 GN = | 0 | 1 | 0 | 0.0 | 4.7 |
| | CDYL - [CDYL_HUMAN] | | | | | |
| Q9Y3P9 | Rab GTPase-activating protein 1 OS = Homo sapiens OX = 9606 GN = RABGAP1 PE = 1 | 0 | 1 | 0 | 0.0 | 1.9 |
| | SV = 3 - [RBGP1_HUMAN] | | | | | |
| Q9Y6B6 | GTP-binding protein SAR1b OS = Homo sapiens OX = 9606 GN = SAR1B PE = 1 SV = | 0 | 1 | 0 | 0.0 | 6.4 |
| | 1 - [SAR1B_HUMAN] | | | | | |
| Q9NZM1-2 | Isoform 2 of Myoferlin OS = Homo sapiens OX = 9606 GN = MYOF - [MYOF_HUMAN] | 3 | 1 | 0 | 2.4 | 0.8 |
| O95391 | Pre-mRNA-splicing factor SLU7 OS = Homo sapiens OX = 9606 GN = SLU7 PE = 1 SV = | 1 | 1 | 0 | 2.0 | 3.5 |
| | 2 - [SLU7_HUMAN] | | | | | |
| Q15208 | Serine/threonine-protein kinase 38 OS = Homo sapiens OX = 9606 GN = STK38 PE = 1 | 1 | 1 | 0 | 2.0 | 2.8 |
| | SV = 1 - [STK38_HUMAN] | | | | | |
| Q6IN84 | rRNA methyltransferase 1, mitochondrial OS = Homo sapiens OX = 9606 GN = MRM1 PE = 1 | 1 | 1 | 0 | 3.4 | 3.4 |
| | SV = 1 - [MRM1_HUMAN] | | | | | |
| Q96SU4-5 | Isoform 5 of Oxysterol-binding protein-related protein 9 OS = Homo sapiens OX = 9606 | 1 | 1 | 0 | 1.4 | 1.4 |
| | GN = OSBPL9 - [OSBL9_HUMAN] | | | | | |
| Q9NX46 | ADP-ribose glycohydrolase ARH3 OS = Homo sapiens OX = 9606 GN = ADPRHL2 PE = 1 | 1 | 1 | 0 | 2.1 | 3.4 |
| | SV = 1 - [ARHL2_HUMAN] | | | | | |
| O60547-2 | Isoform 2 of GDP-mannose 4,6 dehydratase OS = Homo sapiens OX = 9606 GN = | 0 | 1 | 0 | 0.0 | 2.6 |
| | GMDS - [GMDS_HUMAN] | | | | | |
| P09001 | 39S ribosomal protein L3, mitochondrial OS = Homo sapiens OX = 9606 GN = MRPL3 PE = 1 | 0 | 1 | 0 | 0.0 | 3.9 |
| | SV = 1 - [RM03_HUMAN] | | | | | |
| P20618 | Proteasome subunit beta type-1 OS = Homo sapiens OX = 9606 GN = PSMB1 PE = | 0 | 1 | 0 | 0.0 | 5.5 |
| | 1 SV = 2 - [PSB1_HUMAN] | | | | | |
| P98082-3 | Isoform 3 of Disabled homolog 2 OS = Homo sapiens OX = 9606 GN = DAB2 - [DAB2_HUMAN] | 0 | 1 | 0 | 0.0 | 2.0 |
| Q00537 | Cyclin-dependent kinase 17 OS = Homo sapiens OX = 9606 GN = CDK17 PE = 1 SV = | 0 | 1 | 0 | 0.0 | 1.0 |
| | 2 - [CDK17_HUMAN] | | | | | |
| Q04323 | UBX domain-containing protein 1 OS = Homo sapiens OX = 9606 GN = UBXN1 PE = 1 SV = | 0 | 1 | 0 | 0.0 | 7.5 |
| | 2 - [UBXN1_HUMAN] | | | | | |
| Q12899 | Tripartite motif-containing protein 26 OS = Homo sapiens OX = 9606 GN = TRIM26 PE = | 0 | 1 | 0 | 0.0 | 2.3 |
| | 1 SV = 1 - [TRI26_HUMAN] | | | | | |
| Q13057 | Bifunctional coenzyme A synthase OS = Homo sapiens OX = 9606 GN = COASY PE = 1 SV = | 0 | 1 | 0 | 0.0 | 2.0 |
| | 4 - [COASY_HUMAN] | | | | | |
| Q13126-4 | Isoform 4 of S-methyl-5′-thioadenosine phosphorylase OS = Homo sapiens OX = 9606 GN = | 0 | 1 | 0 | 0.0 | 4.9 |
| | MTAP - [MTAP_HUMAN] | | | | | |
| Q13564-3 | Isoform 3 of NEDD8-activating enzyme E1 regulatory subunit OS = Homo sapiens OX = | 0 | 1 | 0 | 0.0 | 2.9 |
| | 9606 GN = NAE1 - [ULA1_HUMAN] | | | | | |
| Q7L099-2 | Isoform 2 of Protein RUFY3 OS = Homo sapiens OX = 9606 GN = RUFY3 - [RUFY3_HUMAN] | 0 | 1 | 0 | 0.0 | 2.3 |
| Q9NRG9-2 | Isoform 2 of Aladin OS = Homo sapiens OX = 9606 GN = AAAS - [AAAS_HUMAN] | 0 | 1 | 0 | 0.0 | 2.8 |
| Q9ULV3-5 | Isoform 5 of Cip1-interacting zinc finger protein OS = Homo sapiens OX = 9606 GN = | 0 | 1 | 0 | 0.0 | 1.3 |
| | CIZ1 - [CIZ1_HUMAN] | | | | | |
| Q9Y6C9 | Mitochondrial carrier homolog 2 OS = Homo sapiens OX = 9606 GN = MTCH2 PE = 1 SV = | 0 | 1 | 0 | 0.0 | 3.3 |
| | 1 - [MTCH2_HUMAN] | | | | | |
| O95819-4 | Isoform 4 of Mitogen-activated protein kinase kinase kinase kinase 4 OS = Homo sapiens | 4 | 1 | 0 | 4.3 | 1.1 |
| | OX = 9606 GN = MAP4K4 - [M4K4_HUMAN] | | | | | |
| Q96A49 | Synapse-associated protein 1 OS = Homo sapiens OX = 9606 GN = SYAP1 PE = 1 SV = | 3 | 1 | 0 | 9.5 | 2.4 |
| | 1 - [SYAP1_HUMAN] | | | | | |
| Q9NP81 | Serine -- tRNA ligase, mitochondrial OS = Homo sapiens OX = 9606 GN = SARS2 PE = 1 | 3 | 1 | 0 | 8.5 | 2.1 |
| | SV = 1 - [SYSM_HUMAN] | | | | | |
| O75794 | Cell division cycle protein 123 homolog OS = Homo sapiens OX = 9606 GN = CDC123 PE = 1 | 2 | 1 | 0 | 7.4 | 1.7 |
| | SV = 1 - [CD123_HUMAN] | | | | | |
| P50747 | Biotin -- protein ligase OS = Homo sapiens OX = 9606 GN = HLCS PE = 1 SV = 1 - [BPL1_HUMAN] | 2 | 1 | 0 | 2.7 | 1.2 |
| Q2M218-2 | Isoform 2 of AP2-associated protein kinase 1 OS = Homo sapiens OX = 9606 GN = | 2 | 1 | 0 | 4.9 | 2.5 |
| | AAK1 - [AAK1_HUMAN] | | | | | |
| P57737-3 | Isoform 3 of Coronin-7 OS = Homo sapiens OX = 9606 GN = CORO7 - [CORO7_HUMAN] | 2 | 1 | 0 | 2.7 | 1.1 |
| Q86YZ3 | Hornerin OS = Homo sapiens OX = 9606 GN = HRNR PE = 1 SV = 2 - [HORN_HUMAN] | 2 | 1 | 0 | 1.3 | 0.8 |
| Q96IR7 | 4-hydroxyphenylpyruvate dioxygenase-like protein OS = Homo sapiens OX = 9606 GN = HPDL | 2 | 1 | 0 | 7.7 | 3.3 |
| | PE = 1 SV = 1 - [HPDL_HUMAN] | | | | | |
| P31350 | Ribonucleoside-diphosphate reductase subunit M2 OS = Homo sapiens OX = 9606 GN = RRM2 | 1 | 1 | 0 | 5.2 | 2.7 |
| | PE = 1 SV = 1 - [RIR2_HUMAN] | | | | | |
| P51114-3 | Isoform 3 of Fragile X mental retardation syndrome-related protein 1 OS = Homo sapiens | 1 | 1 | 0 | 6.0 | 3.0 |
| | OX = 9606 GN = FXR1 - [FXR1_HUMAN] | | | | | |
| Q53EP0 | Fibronectin type Ill domain-containing protein 3B OS = Homo sapiens OX = 9606 GN = | 1 | 1 | 0 | 1.8 | 1.0 |
| | FNDC3B PE = 1 SV = 2 - [FND3B_HUMAN] | | | | | |
| Q92888-2 | Isoform 2 of Rho guanine nucleotide exchange factor 1 OS = Homo sapiens OX = 9606 GN = | 1 | 1 | 0 | 1.8 | 1.1 |
| | ARHGEF1 - [ARHG1_HUMAN] | | | | | |
| Q96A35 | 39S ribosomal protein L24, mitochondrial OS = Homo sapiens OX = 9606 GN = MRPL24 PE = | 1 | 1 | 0 | 11.4 | 5.7 |
| | 1 SV = 1 - [RM24_HUMAN] | | | | | |
| Q96T60 | Bifunctional polynucleotide phosphatase/kinase OS = Homo sapiens OX = 9606 GN = PNKP PE = | 1 | 1 | 0 | 4.5 | 2.3 |
| | 1 SV = 1 - [PNKP_HUMAN] | | | | | |
| Q9H992-2 | Isoform 2 of E3 ubiquitin-protein ligase MARCH7 OS = Homo sapiens OX = 9606 GN = | 1 | 1 | 0 | 2.4 | 1.8 |
| | MARCH7 - [MARH7_HUMAN] | | | | | |
| P18074-2 | Isoform 2 of General transcription and DNA repair factor IIH helicase subunit XPD OS = | 1 | 1 | 0 | 3.3 | 3.3 |
| | Homo sapiens OX = 9606 GN = ERCC2 - [ERCC2_HUMAN] | | | | | |
| P60228 | Eukaryotic translation initiation factor 3 subunit E OS = Homo sapiens OX = 9606 GN = | 1 | 1 | 0 | 2.2 | 2.2 |
| | EIF3E PE = 1 SV = 1 - [EIF3E_HUMAN] | | | | | |
| Q9BQL6 | Fermitin family homolog 1 OS = Homo sapiens OX = 9606 GN = FERMT1 PE = 1 SV = | 1 | 1 | 0 | 2.1 | 1.2 |
| | 1 - [FERM1_HUMAN] | | | | | |
| Q9P2R3 | Rabankyrin-5 OS = Homo sapiens OX = 9606 GN = ANKFY1 PE = 1 SV = 2 - [ANFY1_HUMAN] | 1 | 1 | 0 | 1.1 | 0.5 |
| Q9Y2I7 | 1-phosphatidylinositol 3-phosphate 5-kinase OS = Homo sapiens OX = 9606 GN = PIKFYVE PE = | 1 | 1 | 0 | 0.4 | 0.4 |
| | 1 SV = 3 - [FYV1_HUMAN] | | | | | |
| O43314-2 | Isoform 2 of Inositol hexakisphosphate and diphosphoinositol-pentakisphosphate kinase 2 | 1 | 1 | 0 | 0.8 | 1.0 |
| | OS = Homo sapiens OX = 9606 GN = PPIP5K2 - [VIP2_HUMAN] | | | | | |
| O75616 | GTPase Era, mitochondrial OS = Homo sapiens OX = 9606 GN = ERAL1 PE = 1 SV = | 1 | 1 | 0 | 3.1 | 1.9 |
| | 2 - [ERAL1_HUMAN] | | | | | |
| P21281 | V-type proton ATPase subunit B, brain isoform OS = Homo sapiens OX = 9606 GN = ATP6V1B2 | 1 | 1 | 0 | 2.2 | 2.4 |
| | PE = 1 SV = 3 - [VATB2_HUMAN] | | | | | |
| P29966 | Myristoylated alanine-rich C-kinase substrate OS = Homo sapiens OX = 9606 GN = MARCKS | 1 | 1 | 0 | 3.8 | 3.8 |
| | PE = 1 SV = 4 - [MARCS_HUMAN] | | | | | |
| P49593-2 | Isoform 2 of Protein phosphatase 1F OS = Homo sapiens OX = 9606 GN = PPM1F - [PPM1F_HUMAN] | 1 | 1 | 0 | 2.9 | 3.7 |
| P49642 | DNA primase small subunit OS = Homo sapiens OX = 9606 GN = PRIM1 PE = 1 SV = | 1 | 1 | 0 | 1.7 | 1.7 |
| | 1 - [PRI1_HUMAN] | | | | | |
| P50570-2 | Isoform 2 of Dynamin-2 OS = Homo sapiens OX = 9606 GN = DNM2 - [DYN2_HUMAN] | 1 | 1 | 0 | 1.1 | 0.5 |
| P53814-5 | Isoform B2 of Smoothelin OS = Homo sapiens OX = 9606 GN = SMTN - [SMTN_HUMAN] | 1 | 1 | 0 | 0.8 | 0.8 |
| Q01433-2 | Isoform Ex1A-2-3 of AMP deaminase 2 OS = Homo sapiens OX = 9606 GN = AMPD2 - [AMPD2_HUMAN] | 1 | 1 | 0 | 0.9 | 1.3 |
| Q15418-4 | Isoform 4 of Ribosomal protein S6 kinase alpha-1 OS = Homo sapiens OX = 9606 GN = | 1 | 1 | 0 | 2.2 | 2.1 |
| | RPS6KA1 - [KS6A1_HUMAN] | | | | | |
| Q16881-5 | Isoform 5 of Thioredoxin reductase 1, cytoplasmic OS = Homo sapiens OX = 9606 GN = | 1 | 1 | 0 | 2.1 | 2.1 |
| | TXNRD1 - [TRXR1_HUMAN] | | | | | |
| Q6IAN0 | Dehydrogenase/reductase SDR family member 7B OS = Homo sapiens OX = 9606 GN = DHRS7B | 1 | 1 | 0 | 2.8 | 2.9 |
| | PE = 1 SV = 2 - [DRS7B_HUMAN] | | | | | |
| Q7L0Y3 | tRNA methyltransferase 10 homolog C OS = Homo sapiens OX = 9606 GN = TRMT10C PE = 1 | 1 | 1 | 0 | 1.7 | 1.7 |
| | SV = 2 - [TM10C_HUMAN] | | | | | |
| Q8WVX9 | Fatty acyl-CoA reductase 1 OS = Homo sapiens OX = 9606 GN = FAR1 PE = 1 SV = | 1 | 1 | 0 | 1.7 | 2.1 |
| | 1 - [FACR1_HUMAN] | | | | | |
| Q8WWC4 | m-AAA protease-interacting protein 1, mitochondrial OS = Homo sapiens OX = 9606 | 1 | 1 | 0 | 2.8 | 2.8 |
| | GN = MAIP1 PE = 1 SV = 1 - [MAIP1_HUMAN] | | | | | |
| Q96R06 | Sperm-associated antigen 5 OS = Homo sapiens OX = 9606 GN = SPAG5 PE = 1 | 1 | 1 | 0 | 0.8 | 0.7 |
| | SV = 2 - [SPAG5_HUMAN] | | | | | |
| Q96S44 | EKC/KEOPS complex subunit TP53RK OS = Homo sapiens OX = 9606 GN = TP53RK PE = 1 | 1 | 1 | 0 | 4.0 | 3.6 |
| | SV = 2 - [PRPK_HUMAN] | | | | | |
| Q99805 | Transmembrane 9 superfamily member 2 OS = Homo sapiens OX = 9606 GN = TM9SF2 PE = 1 | 1 | 1 | 0 | 1.9 | 1.7 |
| | SV = 1 - [TM9S2_HUMAN] | | | | | |
| Q9BT92 | Trichoplein keratin filament-binding protein OS = Homo sapiens OX = 9606 GN = TCHP | 1 | 1 | 0 | 1.3 | 1.3 |
| | PE = 1 SV = 1 - [TCHP_HUMAN] | | | | | |
| Q9H479 | Fructosamine-3-kinase OS = Homo sapiens OX = 9606 GN = FN3K PE = 1 SV = 1 - [FN3K_HUMAN] | 1 | 1 | 0 | 2.8 | 3.0 |
| Q9NVC6 | Mediator of RNA polymerase Il transcription subunit 17 OS = Homo sapiens OX = 9606 | 1 | 1 | 0 | 1.6 | 1.6 |
| | GN = MED17 PE = 1 SV = 2 - [MED17_HUMAN] | | | | | |
| Q9UM00-1 | Isoform 3 of Calcium load-activated calcium channel OS = Homo sapiens OX = 9606 | 1 | 1 | 0 | 4.1 | 4.1 |
| | GN = TMCO1 - [TMCO1_HUMAN] | | | | | |
| A6NNY8 | Ubiquitin carboxyl-terminal hydrolase 27 OS = Homo sapiens OX = 9606 GN = USP27X PE = 1 | 0 | 1 | 0 | 0.0 | 1.9 |
| | SV = 3 - [UBP27_HUMAN] | | | | | |
| O00186 | Syntaxin-binding protein 3 OS = Homo sapiens OX = 9606 GN = STXBP3 PE = 1 | 0 | 1 | 0 | 0.0 | 1.0 |
| | SV = 2 - [STXB3_HUMAN] | | | | | |
| O15066 | Kinesin-like protein KIF3B OS = Homo sapiens OX = 9606 GN = KIF3B PE = 1 | 0 | 1 | 0 | 0.0 | 1.3 |
| | SV = 1 - [KIF3B_HUMAN] | | | | | |
| O15294-3 | Isoform 1 of UDP-N-acetylglucosamine -- peptide N-acetylglucosaminyltransferase 110 | 0 | 1 | 0 | 0.0 | 0.7 |
| | kDasubunit OS = Homo sapiens OX = 9606 GN = OGT - [OGT1_HUMAN] | | | | | |
| O75879 | Glutamyl-tRNA(GIn) amidotransferase subunit B, mitochondrial OS = Homo sapiens OX = | 0 | 1 | 0 | 0.0 | 1.3 |
| | 9606 GN = GATB PE = 1 SV = 1 - [GATB_HUMAN] | | | | | |
| O95835-2 | Isoform 2 of Serine/threonine-protein kinase LATS1 OS = Homo sapiens OX = 9606 GN = | 0 | 1 | 0 | 0.0 | 2.0 |
| | LATS1 - [LATS1_HUMAN] | | | | | |
| O95983-2 | Isoform 2 of Methyl-CpG-binding domain protein 3 OS = Homo sapiens OX = 9606 GN = | 0 | 1 | 0 | 0.0 | 3.2 |
| | MBD3 - [MBD3_HUMAN] | | | | | |
| O95985-3 | Isoform 3 of DNA topoisomerase 3-beta-1 OS = Homo sapiens OX = 9606 GN = | 0 | 1 | 0 | 0.0 | 1.6 |
| | TOP3B - [TOP3B_HUMAN] | | | | | |
| P05026-2 | Isoform 2 of Sodium/potassium-transporting ATPase subunit beta-1 OS = Homo sapiens OX = | 0 | 1 | 0 | 0.0 | 3.0 |
| | 9606 GN = ATP1B1 - [AT1B1_HUMAN] | | | | | |
| P06858 | Lipoprotein lipase OS = Homo sapiens OX = 9606 GN = LPL PE = 1 SV = 1 - [LIPL_HUMAN] | 0 | 1 | 0 | 0.0 | 1.9 |
| P19447 | General transcription and DNA repair factor IIH helicase subunit XPB OS = Homo sapiens | 0 | 1 | 0 | 0.0 | 2.2 |
| | OX = 9606 GN = ERCC3 PE = 1 SV = 1 - [ERCC3_HUMAN] | | | | | |
| P19838 | Nuclear factor NF-kappa-B p105 subunit OS = Homo sapiens OX = 9606 GN = NFKB1 PE = | 0 | 1 | 0 | 0.0 | 1.0 |
| | 1 SV = 2 - [NFKB1_HUMAN] | | | | | |
| P21266 | Glutathione S-transferase Mu 3 OS = Homo sapiens OX = 9606 GN = GSTM3 PE = 1 SV = | 0 | 1 | 0 | 0.0 | 1.6 |
| | 3 - [GSTM3_HUMAN] | | | | | |
| P28074 | Proteasome subunit beta type-5 OS = Homo sapiens OX = 9606 GN = PSMB5 PE = 1 SV = | 0 | 1 | 0 | 0.0 | 2.9 |
| | 3 - [PSB5_HUMAN] | | | | | |
| P33897 | ATP-binding cassette sub-family D member 1 OS = Homo sapiens OX = 9606 GN = ABCD1 PE = 1 | 0 | 1 | 0 | 0.0 | 1.5 |
| | SV = 2 - [ABCD1_HUMAN] | | | | | |
| P39019 | 40S ribosomal protein S19 OS = Homo sapiens OX = 9606 GN = RPS19 PE = 1 SV = | 0 | 1 | 0 | 0.0 | 4.6 |
| | 2 - [RS19_HUMAN] | | | | | |
| P51570 | Galactokinase OS = Homo sapiens OX = 9606 GN = GALK1 PE = 1 SV = 1 - [GALK1_HUMAN] | 0 | 1 | 0 | 0.0 | 1.0 |
| P55010 | Eukaryotic translation initiation factor 5 05 = Homo sapiens OX = 9606 GN = EIF5 PE = 1 | 0 | 1 | 0 | 0.0 | 2.0 |
| | SV = 2 - [IF5_HUMAN] | | | | | |
| Q05932-3 | Isoform 3 of Folylpolyglutamate synthase, mitochondrial OS = Homo sapiens OX = 9606 | 0 | 1 | 0 | 0.0 | 2.4 |
| | GN = FPGS - [FOLC_HUMAN] | | | | | |
| Q12933-4 | Isoform 4 of TNF receptor-associated factor 2 OS = Homo sapiens OX = 9606 GN = | 0 | 1 | 0 | 0.0 | 1.5 |
| | TRAF2 - [TRAF2_HUMAN] | | | | | |
| Q13371-2 | Isoform 2 of Phosducin-like protein OS = Homo sapiens OX = 9606 GN = PDCL - [PHLP_HUMAN] | 0 | 1 | 0 | 0.0 | 4.3 |
| Q13442 | 28 kDa heat- and acid-stable phosphoprotein OS = Homo sapiens OX = 9606 GN = PDAP1 | 0 | 1 | 0 | 0.0 | 2.8 |
| | PE = 1 SV = 1 - [HAP28_HUMAN] | | | | | |
| Q14444-2 | Isoform 2 of Caprin-1 OS = Homo sapiens OX = 9606 GN = CAPRIN1 - [CAPR1_HUMAN] | 0 | 1 | 0 | 0.0 | 1.1 |
| Q6P996-3 | Isoform 3 of Pyridoxal-dependent decarboxylase domain-containing protein 1 OS = | 0 | 1 | 0 | 0.0 | 2.6 |
| | Homo sapiens OX = 9606 GN = PDXDC1 - [PDXD1_HUMAN] | | | | | |
| Q6PL18 | ATPase family AAA domain-containing protein 2 OS = Homo sapiens OX = 9606 GN = | 0 | 1 | 0 | 0.0 | 0.6 |
| | ATAD2 PE = 1 SV = 1 - [ATAD2_HUMAN] | | | | | |
| Q7Z3K3-5 | Isoform 5 of Pogo transposable element with ZNF domain OS = Homo sapiens OX = | 0 | 1 | 0 | 0.0 | 0.6 |
| | 9606 GN = POGZ - [POGZ_HUMAN] | | | | | |
| Q86U44 | N6-adenosine-methyltransferase catalytic subunit OS = Homo sapiens OX = 9606 GN = | 0 | 1 | 0 | 0.0 | 1.4 |
| | METTL3 PE = 1 SV = 2 - [MTA70_HUMAN] | | | | | |
| Q8IWA4 | Mitofusin-1 OS = Homo sapiens OX = 9606 GN = MFN1 PE = 1 SV = 3 - [MFN1_HUMAN] | 0 | 1 | 0 | 0.0 | 1.1 |
| Q8N5A5-3 | Isoform 3 of Zinc finger CCCH-type with G patch domain-containing protein OS = | 0 | 1 | 0 | 0.0 | 1.8 |
| | Homo sapiens OX = 9606 GN = ZGPAT - [ZGPAT_HUMAN] | | | | | |
| Q8N9M1-3 | Isoform 3 of Uncharacterized protein C19orf47 OS = Homo sapiens OX = 9606 GN = | 0 | 1 | 0 | 0.0 | 3.2 |
| | C19orf47 - [CS047_HUMAN] | | | | | |
| Q8NCN5 | Pyruvate dehydrogenase phosphatase regulatory subunit, mitochondrial OS = | 0 | 1 | 0 | 0.0 | 1.1 |
| | Homo sapiens OX = 9606 GN = PDPR PE = 1 SV = 2 - [PDPR_HUMAN] | | | | | |
| Q8NEY1-5 | Isoform 5 of Neuron navigator 1 OS = Homo sapiens OX = 9606 GN = NAV1 - [NAV1_HUMAN] | 0 | 1 | 0 | 0.0 | 0.7 |
| Q8NHH9-3 | Isoform 3 of Atlastin-2 OS = Homo sapiens OX = 9606 GN = ATL2 - [ATLA2_HUMAN] | 0 | 1 | 0 | 0.0 | 2.6 |
| Q8WUB8-3 | Isoform 3 of PHD finger protein 10 OS = Homo sapiens OX = 9606 GN = | 0 | 1 | 0 | 0.0 | 1.3 |
| | PHF10 - [PHF10_HUMAN] | | | | | |
| Q93074-3 | Isoform 3 of Mediator of RNA polymerase Il transcription subunit 12 OS = Homo sapiens | 0 | 1 | 0 | 0.0 | 0.4 |
| | OX = 9606 GN = MED12 - [MED12_HUMAN] | | | | | |
| Q969Z0 | FAST kinase domain-containing protein 4 OS = Homo sapiens OX = 9606 GN = TBRG4 PE = 1 | 0 | 1 | 0 | 0.0 | 1.5 |
| | SV = 1 - [FAKD4_HUMAN] | | | | | |
| Q96AG3-3 | Isoform 3 of Solute carrier family 25 member 46 OS = Homo sapiens OX = 9606 | 0 | 1 | 0 | 0.0 | 3.8 |
| | GN = SLC25A46 - [S2546_HUMAN] | | | | | |
| Q96C19 | EF-hand domain-containing protein D2 OS = Homo sapiens OX = 9606 GN = EFHD2 PE = 1 | 0 | 1 | 0 | 0.0 | 2.4 |
| | SV = 1 - [EFHD2_HUMAN] | | | | | |
| Q96FZ2 | Abasic site processing protein HMCES OS = Homo sapiens OX = 9606 GN = HMCES PE = 1 | 0 | 1 | 0 | 0.0 | 2.3 |
| | SV = 1 - [HMCES_HUMAN] | | | | | |
| Q9BQA1-2 | Isoform 2 of Methylosome protein 50 OS = Homo sapiens OX = 9606 GN = | 0 | 1 | 0 | 0.0 | 2.6 |
| | WDR77 - [MEP50_HUMAN] | | | | | |
| Q9BQI3-2 | Isoform 2 of Eukaryotic translation initiation factor 2-alpha kinase 1 OS = | 0 | 1 | 0 | 0.0 | 1.3 |
| | Homo sapiens OX = 9606 GN = EIF2AK1 - [E2AK1_HUMAN] | | | | | |
| Q9BRZ2 | E3 ubiquitin-protein ligase TRIM56 OS = Homo sapiens OX = 9606 GN = TRIM56 PE = 1 | 0 | 1 | 0 | 0.0 | 1.5 |
| | SV = 3 - [TRI56_HUMAN] | | | | | |
| Q9BTC8-2 | Isoform 2 of Metastasis-associated protein MTA3 OS = Homo sapiens OX = 9606 GN = | 0 | 1 | 0 | 0.0 | 1.6 |
| | MTA3 - [MTA3_HUMAN] | | | | | |
| Q9BUB7 | Transmembrane protein 70, mitochondrial OS = Homo sapiens OX = 9606 GN = TMEM70 | 0 | 1 | 0 | 0.0 | 2.6 |
| | PE = 1 SV = 2 - [TMM70_HUMAN] | | | | | |
| Q9BUN8-2 | Isoform 2 of Derlin-1 OS = Homo sapiens OX = 9606 GN = DERL1 - [DERL1_HUMAN] | 0 | 1 | 0 | 0.0 | 2.7 |
| Q9C0D2 | Centrosomal protein of 295 kDa OS = Homo sapiens OX = 9606 GN = CEP295 PE = 1 | 0 | 1 | 0 | 0.0 | 0.3 |
| | SV = 4 - [CE295_HUMAN] | | | | | |
| Q9H3M7-2 | Isoform 2 of Thioredoxin-interacting protein OS = Homo sapiens OX = 9606 GN = | 0 | 1 | 0 | 0.0 | 2.9 |
| | TXNIP - [TXNIP_HUMAN] | | | | | |
| Q9H4H8-2 | Isoform 2 of Protein FAM83D OS = Homo sapiens OX = 9606 GN = FAM83D - [FA83D_HUMAN] | 0 | 1 | 0 | 0.0 | 1.6 |
| Q9H6R4-4 | Isoform 4 of Nucleolar protein 6 OS = Homo sapiens OX = 9606 GN = NOL6 - [NOL6_HUMAN] | 0 | 1 | 0 | 0.0 | 0.7 |
| Q9H788-2 | Isoform 2 of SH2 domain-containing protein 4A OS = Homo sapiens OX = 9606 GN = | 0 | 1 | 0 | 0.0 | 2.0 |
| | SH2D4A - [SH24A_HUMAN] | | | | | |
| Q9H936 | Mitochondrial glutamate carrier 1 OS = Homo sapiens OX = 9606 GN = SLC25A22 PE = 1 | 0 | 1 | 0 | 0.0 | 1.8 |
| | SV = 1 - [GHC1_HUMAN] | | | | | |
| Q9HAD4 | WD repeat-containing protein 41 OS = Homo sapiens OX = 9606 GN = WDR41 PE = 1 | 0 | 1 | 0 | 0.0 | 2.5 |
| | SV = 3 - [WDR41_HUMAN] | | | | | |
| Q9NP97 | Dynein light chain roadblock-type 1 OS = Homo sapiens OX = 9606 GN = DYNLRB1 PE = 1 | 0 | 1 | 0 | 0.0 | 11.5 |
| | SV = 3 - [DLRB1_HUMAN] | | | | | |
| Q9NUL3-4 | Isoform 4 of Double-stranded RNA-binding protein Staufen homolog 2 OS = Homo sapiens | 0 | 1 | 0 | 0.0 | 2.8 |
| | OX = 9606 GN = STAU2 - [STAU2_HUMAN] | | | | | |
| Q9NUQ7 | Ufm1-specific protease 2 OS = Homo sapiens OX = 9606 GN = UFSP2 PE = 1 SV = | 0 | 1 | 0 | 0.0 | 2.6 |
| | 3 - [UFSP2_HUMAN] | | | | | |
| Q9NVH1-3 | Isoform 3 of DnaJ homolog subfamily C member 11 OS = Homo sapiens OX = 9606 GN = | 0 | 1 | 0 | 0.0 | 1.8 |
| | DNAJC11 - [DJC11_HUMAN] | | | | | |
| Q9NWU5-2 | Isoform 2 of 39S ribosomal protein L22, mitochondrial OS = Homo sapiens OX = 9606 | 0 | 1 | 0 | 0.0 | 5.3 |
| | GN = MRPL22 - [RM22_HUMAN] | | | | | |
| Q9NWY4 | Histone PARylation factor 1 OS = Homo sapiens OX = 9606 GN = HPF1 PE = 1 SV = | 0 | 1 | 0 | 0.0 | 2.4 |
| | 2 - [HPF1_HUMAN] | | | | | |
| Q9NZL9-4 | Isoform 4 of Methionine adenosyltransferase 2 subunit beta OS = Homo sapiens OX = | 0 | 1 | 0 | 0.0 | 2.3 |
| | 9606 GN = MAT2B - [MAT2B_HUMAN] | | | | | |
| Q9P0W2-2 | Isoform 2 of SWI/SNF-related matrix-associated actin-dependent regulator of chromatin | 0 | 1 | 0 | 0.0 | 2.6 |
| | subfamily E member 1-related OS = Homo sapiens OX = 9606 GN = HMG20B - [HM20B_HUMAN] | | | | | |
| Q9P2Y4 | Zinc finger protein 219 OS = Homo sapiens OX = 9606 GN = ZNF219 PE = 1 | 0 | 1 | 0 | 0.0 | 1.8 |
| | SV = 2 - [ZN219_HUMAN] | | | | | |
| Q9UKY7-2 | Isoform 2 of Protein CDV3 homolog OS = Homo sapiens OX = 9606 GN = CDV3 - [CDV3_HUMAN] | 0 | 1 | 0 | 0.0 | 6.4 |
| Q9UMX1 | Suppressor of fused homolog OS = Homo sapiens OX = 9606 GN = SUFU PE = 1 | 0 | 1 | 0 | 0.0 | 2.3 |
| | SV = 2 - [SUFU_HUMAN] | | | | | |
| Q9Y2Z2-2 | Isoform 1 of Protein MTO1 homolog, mitochondrial OS = Homo sapiens OX = 9606 | 0 | 1 | 0 | 0.0 | 1.4 |
| | GN = MTO1 - [MTO1_HUMAN] | | | | | |
| Q9Y3S2 | Zinc finger protein 330 OS = Homo sapiens OX = 9606 GN = ZNF330 PE = 1 SV = | 0 | 1 | 0 | 0.0 | 2.6 |
| | 1 - [ZN330_HUMAN] | | | | | |
| Q9Y6V7 | Probable ATP-dependent RNA helicase DDX49 OS = Homo sapiens OX = 9606 GN = DDX49 | 0 | 1 | 0 | 0.0 | 2.9 |
| | PE = 1 SV = 1 - [DDX49_HUMAN] | | | | | |
| P18031 | Tyrosine-protein phosphatase non-receptor type 1 OS = Homo sapiens OX = 9606 | 5 | 0 | 0 | 11.9 | 0.0 |
| | GN = PTPN1 PE = 1 SV = 1 - [PTN1_HUMAN] | | | | | |
| P30837 | Aldehyde dehydrogenase X, mitochondrial OS = Homo sapiens OX = 9606 GN = ALDH1B1 | 5 | 0 | 0 | 8.1 | 0.0 |
| | PE = 1 SV = 3 - [AL1B1_HUMAN] | | | | | |
| Q9BVL4 | Protein adenylyltransferase SeIO, mitochondrial OS = Homo sapiens OX = 9606 | 4 | 0 | 0 | 5.8 | 0.0 |
| | GN = SELENOO PE = 1 SV = 3 - [SELO_HUMAN] | | | | | |
| Q17RY0-2 | Isoform 2 of Cytoplasmic polyadenylation element-binding protein 4 OS = Homo sapiens | 3 | 0 | 0 | 3.9 | 0.0 |
| | OX = 9606 GN = CPEB4 - [CPEB4_HUMAN] | | | | | |
| Q92890 | Ubiquitin recognition factor in ER-associated degradation protein 1 OS = Homo sapiens | 2 | 0 | 0 | 10.0 | 0.0 |
| | OX = 9606 GN = UFD1 PE = 1 SV = 3 - [UFD1_HUMAN] | | | | | |
| P46783 | 40S ribosomal protein S10 OS = Homo sapiens OX = 9606 GN = RPS10 PE = 1 | 2 | 0 | 0 | 11.5 | 0.0 |
| | SV = 1 - [RS10_HUMAN] | | | | | |
| Q6YP21-3 | Isoform 3 of Kynurenine -- oxoglutarate transaminase 3 OS = Homo sapiens OX = 9606 | 2 | 0 | 0 | 7.6 | 0.0 |
| | GN = KYAT3 - [KAT3_HUMAN] | | | | | |
| Q8WUF5 | RelA-associated inhibitor OS = Homo sapiens OX = 9606 GN = PPP1R13L PE = 1 | 2 | 0 | 0 | 3.6 | 0.0 |
| | SV = 4 - [IASPP_HUMAN] | | | | | |
| Q9Y6Y0 | Influenza virus NS1A-binding protein OS = Homo sapiens OX = 9606 GN = IVNS1ABP | 2 | 0 | 0 | 3.2 | 0.0 |
| | PE = 1 SV = 3 - [NS1BP_HUMAN] | | | | | |
| E9PRG8 | Uncharacterized protein C11orf98 OS = Homo sapiens OX = 9606 GN = C11orf98 PE = 4 | 2 | 0 | 0 | 15.2 | 0.0 |
| | SV = 2 - [CK098_HUMAN] | | | | | |
| O43896 | Kinesin-like protein KIF1C OS = Homo sapiens OX = 9606 GN = KIF1C PE = 1 | 2 | 0 | 0 | 2.0 | 0.0 |
| | SV = 3 - [KIF1C_HUMAN] | | | | | |
| Q9H267 | Vacuolar protein sorting-associated protein 33B OS = Homo sapiens OX = 9606 GN = | 2 | 0 | 0 | 2.4 | 0.0 |
| | VPS33B PE = 1 SV = 2 - [VP33B_HUMAN] | | | | | |
| P62258-2 | Isoform SV of 14-3-3 protein epsilon OS = Homo sapiens OX = 9606 GN = | 1 | 0 | 0 | 5.2 | 0.0 |
| | YWHAE - [1433E_HUMAN] | | | | | |
| Q99439-2 | Isoform 2 of Calponin-2 OS = Homo sapiens OX = 9606 GN = CNN2 - [CNN2_HUMAN] | 1 | 0 | 0 | 3.2 | 0.0 |
| Q9GZS1 | DNA-directed RNA polymerase I subunit RPA49 OS = Homo sapiens OX = 9606 GN = | 1 | 0 | 0 | 3.5 | 0.0 |
| | POLR1E PE = 1 SV = 3 - [RPA49_HUMAN] | | | | | |
| Q9NYL2 | Mitogen-activated protein kinase kinase kinase 20 OS = Homo sapiens OX = | 1 | 0 | 0 | 1.7 | 0.0 |
| | 9606 GN = MAP3K20 PE = 1 SV = 3 - [M3K20_HUMAN] | | | | | |
| Q9NZJO | Denticleless protein homolog OS = Homo sapiens OX = 9606 GN = DTL PE = 1 | 1 | 0 | 0 | 2.8 | 0.0 |
| | SV = 3 - [DTL_HUMAN] | | | | | |
| Q9UFCO | Leucine-rich repeat and WD repeat-containing protein 1 OS = Homo sapiens | 1 | 0 | 0 | 3.0 | 0.0 |
| | OX = 9606 GN = LRWD1 PE = 1 SV = 2 - [LRWD1_HUMAN] | | | | | |
| Q9Y2U8 | Inner nuclear membrane protein Man1 OS = Homo sapiens OX = 9606 GN = LEMD3 | 1 | 0 | 0 | 2.2 | 0.0 |
| | PE = 1 SV = 2 - [MAN1_HUMAN] | | | | | |
| O00443 | Phosphatidylinositol 4-phosphate 3-kinase C2 domain-containing subunit alpha | 1 | 0 | 0 | 0.8 | 0.0 |
| | [P3C2A_HUMAN] | | | | | |
| Q15070-2 | Isoform 2 of Mitochondrial inner membrane protein OXA1L OS = Homo sapiens OX = 9606 | 1 | 0 | 0 | 2.9 | 0.0 |
| | GN = OXA1L - [OXA1L_HUMAN] | | | | | |
| Q15814 | Tubulin-specific chaperone C OS = Homo sapiens OX = 9606 GN = TBCC PE = | 1 | 0 | 0 | 3.0 | 0.0 |
| | 1 SV = 2 - [TBCC_HUMAN] | | | | | |
| Q5T7W7 | Thiosulfate sulfurtransferase/rhodanese-like domain-containing protein 2 OS = | 1 | 0 | 0 | 2.2 | 0.0 |
| | Homo sapiens OX = 9606 GN = PIK3C2A PE = 1 SV = 2 - [TSTD2_HUMAN] | | | | | |
| Q6P1Q9 | Methyltransferase-like protein 2B OS = Homo sapiens OX = 9606 GN = METTL2B PE = 1 | 1 | 0 | 0 | 3.3 | 0.0 |
| | SV = 3 - [MET2B_HUMAN] | | | | | |
| Q969S9-2 | Isoform 2 of Ribosome-releasing factor 2, mitochondrial OS = Homo sapiens | 1 | 0 | 0 | 1.2 | 0.0 |
| | OX = 9606 GN = GFM2 - [RRF2M_HUMAN] | | | | | |
| Q9BQ70 | Transcription factor 25 OS = Homo sapiens OX = 9606 GN = TCF25 PE = 1 | 1 | 0 | 0 | 2.6 | 0.0 |
| | SV = 1 - [TCF25_HUMAN] | | | | | |
| Q9H1H9-3 | Isoform 3 of Kinesin-like protein KIF13A OS = Homo sapiens OX = 9606 | 1 | 0 | 0 | 0.6 | 0.0 |
| | GN = KIF13A - [KI13A_HUMAN] | | | | | |
| Q9UL54-2 | Isoform 2 of Serine/threonine-protein kinase TAO2 OS = Homo sapiens OX = 9606 | 1 | 0 | 0 | 1.1 | 0.0 |
| | GN = TAOK2 - [TAOK2_HUMAN] | | | | | |
| O15198-2 | Isoform B of Mothers against decapentaplegic homolog 9 OS = Homo sapiens | 1 | 0 | 0 | 1.9 | 0.0 |
| | OX = 9606 GN = SMAD9 - [SMAD9_HUMAN] | | | | | |
| O15270 | Serine palmitoyltransferase 2 OS = Homo sapiens OX = 9606 GN = SPTLC2 PE = 1 | 1 | 0 | 0 | 1.5 | 0.0 |
| | SV = 1 - [SPTC2_HUMAN] | | | | | |
| O43294-2 | Isoform 2 of Transforming growth factor beta-1-induced transcript 1 protein OS = | 1 | 0 | 0 | 1.9 | 0.0 |
| | Homo sapiens OX = 9606 GN = TGFB111 - [TGFI1_HUMAN] | | | | | |
| O43402 | ER membrane protein complex subunit 8 OS = Homo sapiens OX = 9606 GN = EMC8 PE = 1 | 1 | 0 | 0 | 5.6 | 0.0 |
| | SV = 1 - [EMC8_HUMAN] | | | | | |
| O43852-5 | Isoform 5 of Calumenin OS = Homo sapiens OX = 9606 GN = CALU - [CALU_HUMAN] | 1 | 0 | 0 | 3.5 | 0.0 |
| O60256 | Phosphoribosyl pyrophosphate synthase-associated protein 2 OS = Homo sapiens | 1 | 0 | 0 | 3.5 | 0.0 |
| | OX = 9606 GN = PRPSAP2 PE = 1 SV = 1 - [KPRB_HUMAN] | | | | | |
| O60869-2 | Isoform 2 of Endothelial differentiation-related factor 1 OS = Homo sapiens | 1 | 0 | 0 | 5.5 | 0.0 |
| | OX = 9606 GN = EDF1 - [EDF1_HUMAN] | | | | | |
| O75955-2 | Isoform 2 of Flotillin-1 OS = Homo sapiens OX = 9606 GN = FLOT1 - [FLOT1_HUMAN] | 1 | 0 | 0 | 2.8 | 0.0 |
| P06788 | Protein E7 OS = Human papillomavirus type 18 OX = 333761 GN = E7 PE = 1 | 1 | 0 | 0 | 4.1 | 0.0 |
| | SV = 2 - [VE7_HPV18] | | | | | |
| P09429 | High mobility group protein B1 OS = Homo sapiens OX = 9606 GN = HMGB1 PE = 1 | 1 | 0 | 0 | 4.7 | 0.0 |
| | SV = 3 - [HMGB1_HUMAN] | | | | | |
| P17252 | Protein kinase C alpha type OS = Homo sapiens OX = 9606 GN = PRKCA PE = 1 | 1 | 0 | 0 | 1.2 | 0.0 |
| | SV = 4 - [KPCA_HUMAN] | | | | | |
| P35270 | Sepiapterin reductase OS = Homo sapiens OX = 9606 GN = SPR PE = 1 | 1 | 0 | 0 | 4.5 | 0.0 |
| | SV = 1 - [SPRE_HUMAN] | | | | | |
| P38117 | Electron transfer flavoprotein subunit beta OS = Homo sapiens OX = 9606 | 1 | 0 | 0 | 2.7 | 0.0 |
| | GN = ETFB PE = 1 SV = 3 - [ETFB_HUMAN] | | | | | |
| P41214 | Eukaryotic translation initiation factor 2D OS = Homo sapiens OX = 9606 | 1 | 0 | 0 | 1.8 | 0.0 |
| | GN = EIF2D PE = 1 SV = 3 - [EIF2D_HUMAN] | | | | | |
| P51116 | Fragile X mental retardation syndrome-related protein 2 OS = Homo sapiens OX = | 1 | 0 | 0 | 1.2 | 0.0 |
| | 9606 GN = FXR2 PE = 1 SV = 2 - [FXR2_HUMAN] | | | | | |
| P53582 | Methionine aminopeptidase 1 OS = Homo sapiens OX = 9606 GN = METAP1 PE = 1 | 1 | 0 | 0 | 1.9 | 0.0 |
| | SV = 2 - [MAP11_HUMAN] | | | | | |
| P53680-2 | Isoform 2 of AP-2 complex subunit sigma OS = Homo sapiens OX = 9606 | 1 | 0 | 0 | 5.1 | 0.0 |
| | GN = AP2S1 - [AP2S1_HUMAN] | | | | | |
| P78362 | SRSF protein kinase 2 OS = Homo sapiens OX = 9606 GN = SRPK2 PE = 1 | 1 | 0 | 0 | 1.5 | 0.0 |
| | SV = 3 - [SRPK2_HUMAN] | | | | | |
| P82912-2 | Isoform 2 of 28S ribosomal protein S11, mitochondrial OS = Homo sapiens | 1 | 0 | 0 | 4.8 | 0.0 |
| | OX = 9606 GN = MRPS11 - [RT11_HUMAN] | | | | | |
| Q03164-2 | Isoform 2 of Histone-lysine N-methyltransferase 2A OS = Homo sapiens | 1 | 0 | 0 | 0.2 | 0.0 |
| | OX = 9606 GN = KMT2A - [KMT2A_HUMAN] | | | | | |
| Q05682-5 | Isoform 5 of Caldesmon OS = Homo sapiens OX = 9606 GN = CALD1 - [CALD1_HUMAN] | 1 | 0 | 0 | 2.1 | 0.0 |
| Q13332-6 | Isoform 2 of Receptor-type tyrosine-protein phosphatase S OS = Homo sapiens | 1 | 0 | 0 | 0.6 | 0.0 |
| | OX = 9606 GN = PTPRS - [PTPRS_HUMAN] | | | | | |
| Q13555-10 | Isoform 10 of Calcium/calmodulin-dependent protein kinase type II subunit | 1 | 0 | 0 | 1.6 | 0.0 |
| | [gamma OS = Homo sapiens OX = 9606 GN = CAMK2G - KCC2G_HUMAN] | | | | | |
| Q13888 | General transcription factor IIH subunit 2 OS = Homo sapiens OX = 9606 GN = | 1 | 0 | 0 | 1.5 | 0.0 |
| | GTF2H2 PE = 1 SV = 1 - [TF2H2_HUMAN] | | | | | |
| Q14106-2 | Isoform 2 of Protein Tob2 OS = Homo sapiens OX = 9606 GN = TOB2 - [TOB2_HUMAN] | 1 | 0 | 0 | 3.5 | 0.0 |
| Q14116-2 | Isoform 2 of Interleukin-18 OS = Homo sapiens OX = 9606 GN = IL18 - [IL18_HUMAN] | 1 | 0 | 0 | 3.5 | 0.0 |
| Q15036-2 | Isoform 2 of Sorting nexin-17 OS = Homo sapiens OX = 9606 GN = | 1 | 0 | 0 | 2.1 | 0.0 |
| | SNX17 - [SNX17_HUMAN] | | | | | |
| Q15058 | Kinesin-like protein KIF14 OS = Homo sapiens OX = 9606 GN = KIF14 PE = 1 | 1 | 0 | 0 | 0.4 | 0.0 |
| | SV = 1 - [KIF14_HUMAN] | | | | | |
| Q15437 | Protein transport protein Sec23B OS = Homo sapiens OX = 9606 GN = SEC23B | 1 | 0 | 0 | 1.1 | 0.0 |
| | PE = 1 SV = 2 - [SC23B_HUMAN] | | | | | |
| Q5VST9-5 | Isoform 4 of Obscurin OS = Homo sapiens OX = 9606 GN = OBSCN - [OBSCN_HUMAN] | 1 | 0 | 0 | 0.2 | 0.0 |
| Q5VYK3 | Proteasome adapter and scaffold protein ECM29 OS = Homo sapiens OX = 9606 GN = | 1 | 0 | 0 | 0.4 | 0.0 |
| | ECPAS PE = 1 SV = 2 - [ECM29_HUMAN] | | | | | |
| Q5VZL5-2 | Isoform 2 of Zinc finger MYM-type protein 4 OS = Homo sapiens OX = 9606 GN = | 1 | 0 | 0 | 1.3 | 0.0 |
| | ZMYM4 - [ZMYM4_HUMAN] | | | | | |
| Q6P1N0-2 | Isoform 2 of Coiled-coil and C2 domain-containing protein 1A OS = Homo sapiens | 1 | 0 | 0 | 0.9 | 0.0 |
| | OX = 9606 GN = CC2D1A - [C2D1A_HUMAN] | | | | | |
| Q7L7X3-3 | Isoform 3 of Serine/threonine-protein kinase TAO1 OS = Homo sapiens OX = 9606 | 1 | 0 | 0 | 0.9 | 0.0 |
| | GN = TAOK1 - [TAOK1_HUMAN] | | | | | |
| Q86UL3 | Glycerol-3-phosphate acyltransferase 4 OS = Homo sapiens OX = 9606 GN = GPAT4 | 1 | 0 | 0 | 1.8 | 0.0 |
| | PE = 1 SV = 1 - [GPAT4_HUMAN] | | | | | |
| Q86V81 | THO complex subunit 4 OS = Homo sapiens OX = 9606 GN = ALYREF PE = 1 | 1 | 0 | 0 | 3.8 | 0.0 |
| | SV = 3 - [THOC4_HUMAN] | | | | | |
| Q86VM9 | Zinc finger CCCH domain-containing protein 18 OS = Homo sapiens OX = 9606 | 1 | 0 | 0 | 1.2 | 0.0 |
| | GN = ZC3H18 PE = 1 SV = 2 - [ZCH18_HUMAN] | | | | | |
| Q86VP6 | Cullin-associated NEDD8-dissociated protein 1 OS = Homo sapiens OX = 9606 | 1 | 0 | 0 | 0.7 | 0.0 |
| | GN = CAND1 PE = 1 SV = 2 - [CAND1_HUMAN] | | | | | |
| Q86W56 | Poly(ADP-ribose) glycohydrolase OS = Homo sapiens OX = 9606 GN = PARG PE = 1 | 1 | 0 | 0 | 0.8 | 0.0 |
| | SV = 1 - [PARG_HUMAN] | | | | | |
| Q8IZ73-2 | Isoform 2 of RNA pseudouridylate synthase domain-containing protein 2 OS = | 1 | 0 | 0 | 2.5 | 0.0 |
| | Homo sapiens OX = 9606 GN = RPUSD2 - [RUSD2_HUMAN] | | | | | |
| Q8N1G4 | Leucine-rich repeat-containing protein 47 OS = Homo sapiens OX = 9606 GN = | 1 | 0 | 0 | 1.4 | 0.0 |
| | LRRC47 PE = 1 SV = 1 - [LRC47_HUMAN] | | | | | |
| Q8N726-2 | Isoform smARF of Tumor suppressor ARF OS = Homo sapiens OX = 9606 | 1 | 0 | 0 | 10.6 | 0.0 |
| | GN = CDKN2A - [ARF_HUMAN] | | | | | |
| Q8ND56-3 | Isoform 3 of Protein LSM14 homolog A OS = Homo sapiens OX = 9606 GN = | 1 | 0 | 0 | 2.5 | 0.0 |
| | LSM14A - [LS14A_HUMAN] | | | | | |
| Q8NF37 | Lysophosphatidylcholine acyltransferase 1 OS = Homo sapiens OX = 9606 GN = LPCAT1 | 1 | 0 | 0 | 1.3 | 0.0 |
| | PE = 1 SV = 2 - [PCAT1_HUMAN] | | | | | |
| Q8TCG1-2 | Isoform 2 of Protein CIP2A OS = Homo sapiens OX = 9606 GN = CIP2A - [CIP2A_HUMAN] | 1 | 0 | 0 | 1.3 | 0.0 |
| Q8WXG6-6 | Isoform 6 of MAP kinase-activating death domain protein OS = Homo sapiens OX = 9606 | 1 | 0 | 0 | 0.5 | 0.0 |
| | GN = MADD - [MADD_HUMAN] | | | | | |
| Q92538-3 | Isoform 3 of Golgi-specific brefeldin A-resistance guanine nucleotide exchange | 1 | 0 | 0 | 0.5 | 0.0 |
| | factor 1 OS = Homo sapiens OX = 9606 GN = GBF1 - [GBF1_HUMAN] | | | | | |
| Q96RE7 | Nucleus accumbens-associated protein 1 OS = Homo sapiens OX = 9606 GN = NACC1 | 1 | 0 | 0 | 1.2 | 0.0 |
| | PE = 1 SV = 1 - [NACC1_HUMAN] | | | | | |
| Q99497 | Protein/nucleic acid deglycase DJ-1 OS = Homo sapiens OX = 9606 GN = PARK7 | 1 | 0 | 0 | 7.2 | 0.0 |
| | PE = 1 SV = 2 - [PARK7_HUMAN] | | | | | |
| Q9BTL3 | RNA guanine-N7 methyltransferase activating subunit OS = Homo sapiens OX = 9606 | 1 | 0 | 0 | 6.5 | 0.0 |
| | GN = RAMAC PE = 1 SV = 1 - [RAMAC_HUMAN] | | | | | |
| Q9BU76-4 | Isoform 4 of Multiple myeloma tumor-associated protein 2 OS = Homo sapiens | 1 | 0 | 0 | 4.8 | 0.0 |
| | OX = 9606 GN = MMTAG2 - [MMTA2_HUMAN] | | | | | |
| Q9BUH8 | Brain-enriched guanylate kinase-associated protein OS = Homo sapiens OX = 9606 | 1 | 0 | 0 | 1.2 | 0.0 |
| | GN = BEGAIN PE = 1 SV = 1 - [BEGIN_HUMAN] | | | | | |
| Q9BUZ4 | TNF receptor-associated factor 4 OS = Homo sapiens OX = 9606 GN = TRAF4 PE = 1 | 1 | 0 | 0 | 2.0 | 0.0 |
| | SV = 1 - [TRAF4_HUMAN] | | | | | |
| Q9C0J8 | pre-mRNA 3′ end processing protein WDR33 OS = Homo sapiens OX = 9606 GN = WDR33 | 1 | 0 | 0 | 0.5 | 0.0 |
| | PE = 1 SV = 2 - [WDR33_HUMAN] | | | | | |
| Q9H8H0 | Nucleolar protein 11 OS = Homo sapiens OX = 9606 GN = NOL11 PE = 1 | 1 | 0 | 0 | 1.5 | 0.0 |
| | SV = 1 - [NOL11_HUMAN] | | | | | |
| Q9NVZ3-4 | Isoform 4 of Adaptin ear-binding coat-associated protein 2 OS = Homo sapiens | 1 | 0 | 0 | 6.3 | 0.0 |
| | OX = 9606 GN = NECAP2 - [NECP2_HUMAN] | | | | | |
| Q9NYL2-2 | Isoform 2 of Mitogen-activated protein kinase kinase kinase 20 OS = Homo sapiens | 1 | 0 | 0 | 1.5 | 0.0 |
| | OX = 9606 GN = MAP3K20 - [M3K20_HUMAN] | | | | | |
| Q9NZT1 | Calmodulin-like protein 5 OS = Homo sapiens OX = 9606 GN = CALML5 PE = 1 | 1 | 0 | 0 | 7.1 | 0.0 |
| | SV = 2 - [CALL5_HUMAN] | | | | | |
| Q9P2I0 | Cleavage and polyadenylation specificity factor subunit 2 OS = Homo sapiens | 1 | 0 | 0 | 1.1 | 0.0 |
| | OX = 9606 GN = CPSF2 PE = 1 SV = 2 - [CPSF2_HUMAN] | | | | | |
| Q9UJK0 | Ribosome biogenesis protein TSR3 homolog OS = Homo sapiens OX = 9606 | 1 | 0 | 0 | 3.1 | 0.0 |
| | GN = TSR3 PE = 1 SV = 1 - [TSR3_HUMAN] | | | | | |
| Q9UKL0 | REST corepressor 1 OS = Homo sapiens OX = 9606 GN = RCOR1 PE = 1 | 1 | 0 | 0 | 2.1 | 0.0 |
| | SV = 2 - [RCOR1_HUMAN] | | | | | |
| Q9ULV4 | Coronin-1C OS = Homo sapiens OX = 9606 GN = CORO1C PE = 1 SV = 1 - [COR1C_HUMAN] | 1 | 0 | 0 | 1.5 | 0.0 |
| Q9Y2S6 | Translation machinery-associated protein 7 OS = Homo sapiens OX = 9606 | 1 | 0 | 0 | 6.3 | 0.0 |
| | GN = TMA7 PE = 1 SV = 1 - [TMA7_HUMAN] | | | | | |
| Q9Y2Z4 | Tyrosine -- tRNA ligase, mitochondrial OS = Homo sapiens OX = 9606 GN = YARS2 | 1 | 0 | 0 | 2.4 | 0.0 |
| | PE = 1 SV = 2 - [SYYM_HUMAN] | | | | | |
| Q9Y619 | Mitochondrial ornithine transporter 1 OS = Homo sapiens OX = 9606 GN = SLC25A15 | 1 | 0 | 0 | 2.3 | 0.0 |
| | PE = 1 SV = 1 - [ORNT1_HUMAN] | | | | | |
| A0PJW6 | Transmembrane protein 223 OS = Homo sapiens OX = 9606 GN = TMEM223 PE = 1 | 0 | 0 | 0 | 0.0 | 0.0 |
| | SV = 1 - [TM223_HUMAN] | | | | | |
| O00401 | Neural Wiskott-Aldrich syndrome protein OS = Homo sapiens OX = 9606 GN = WASL | 0 | 0 | 0 | 0.0 | 0.0 |
| | PE = 1 SV = 2 - [WASL_HUMAN] | | | | | |
| O00422 | Histone deacetylase complex subunit SAP18 OS = Homo sapiens OX = 9606 GN = | 0 | 0 | 0 | 0.0 | 0.0 |
| | SAP18 PE = 1 SV = 1 - [SAP18_HUMAN] | | | | | |
| O00522 | Krev interaction trapped protein 1 OS = Homo sapiens OX = 9606 GN = KRIT1 | 0 | 0 | 0 | 0.0 | 0.0 |
| | PE = 1 SV = 2 - [KRIT1_HUMAN] | | | | | |
| O14641 | Segment polarity protein dishevelled homolog DVL-2 OS = Homo sapiens OX = | 0 | 0 | 0 | 0.0 | 0.0 |
| | 9606 GN = DVL2 PE = 1 SV = 1 - [DVL2_HUMAN] | | | | | |
| O14802 | DNA-directed RNA polymerase Ill subunit RPC1 OS = Homo sapiens OX = 9606 | 0 | 0 | 0 | 0.0 | 0.0 |
| | GN = POLR3A PE = 1 SV = 2 - [RPC1_HUMAN] | | | | | |
| O14879 | Interferon-induced protein with tetratricopeptide repeats 3 OS = Homo sapiens | 0 | 0 | 0 | 0.0 | 0.0 |
| | OX = 9606 GN = IFIT3 PE = 1 SV = 1 - [IFIT3_HUMAN] | | | | | |
| O14974-5 | Isoform 5 of Protein phosphatase 1 regulatory subunit 12A OS = Homo sapiens | 0 | 0 | 0 | 0.0 | 0.0 |
| | OX = 9606 GN = PPP1R12A - [MYPT1_HUMAN] | | | | | |
| O15111 | Inhibitor of nuclear factor kappa-B kinase subunit alpha OS = Homo sapiens | 0 | 0 | 0 | 0.0 | 0.0 |
| | OX = 9606 GN = CHUK PE = 1 SV = 2 - [IKKA_HUMAN] | | | | | |
| O15118 | NPC intracellular cholesterol transporter 1 OS = Homo sapiens OX = 9606 | 0 | 0 | 0 | 0.0 | 0.0 |
| | GN = NPC1 PE = 1 SV = 2 - [NPC1_HUMAN] | | | | | |
| O15213 | WD repeat-containing protein 46 OS = Homo sapiens OX = 9606 GN = WDR46 | 0 | 0 | 0 | 0.0 | 0.0 |
| | PE = 1 SV = 3 - [WDR46_HUMAN] | | | | | |
| O15533-2 | Isoform 2 of Tapasin OS = Homo sapiens OX = 9606 GN = TAPBP - [TPSN_HUMAN] | 0 | 0 | 0 | 0.0 | 0.0 |
| O43251-6 | Isoform 6 of RNA binding protein fox-1 homolog 2 OS = Homo sapiens OX = 9606 | 0 | 0 | 0 | 0.0 | 0.0 |
| | GN = RBFOX2 - [RFOX2_HUMAN] | | | | | |
| O43426 | Synaptojanin-1 OS = Homo sapiens OX = 9606 GN = SYNJ1 PE = 1 SV = | 0 | 0 | 0 | 0.0 | 0.0 |
| | 2 - [SYNJ1_HUMAN] | | | | | |
| O43707 | Alpha-actinin-4 OS = Homo sapiens OX = 9606 GN = ACTN4 PE = 1 SV = | 0 | 0 | 0 | 0.0 | 0.0 |
| | 2 - [ACTN4_HUMAN] | | | | | |
| O43837-3 | Isoform C of Isocitrate dehydrogenase [NAD] subunit beta, mitochondrial | 0 | 0 | 0 | 0.0 | 0.0 |
| | OS = Homo sapiens OX = 9606 GN = IDH3B - [IDH3B_HUMAN] | | | | | |
| O60216 | Double-strand-break repair protein rad21 homolog OS = Homo sapiens | 0 | 0 | 0 | 0.0 | 0.0 |
| | OX = 9606 GN = RAD21 PE = 1 SV = 2 - [RAD21_HUMAN] | | | | | |
| O60318 | Germinal-center associated nuclear protein OS = Homo sapiens OX = | 0 | 0 | 0 | 0.0 | 0.0 |
| | 9606 GN = MCM3AP PE = 1 SV = 2 - [GANP_HUMAN] | | | | | |
| O60524-4 | Isoform 4 of Nuclear export mediator factor NEMF OS = Homo sapiens | 0 | 0 | 0 | 0.0 | 0.0 |
| | OX = 9606 GN = NEMF - [NEMF_HUMAN] | | | | | |
| O60684 | Importin subunit alpha-7 OS = Homo sapiens OX = 9606 GN = KPNA6 PE = 1 | 0 | 0 | 0 | 0.0 | 0.0 |
| | SV = 1 - [IMA7_HUMAN] | | | | | |
| O60832-2 | Isoform 3 of H/ACA ribonucleoprotein complex subunit DKC1 OS = Homo sapiens | 0 | 0 | 0 | 0.0 | 0.0 |
| | OX = 9606 GN = DKC1 - [DKC1_HUMAN] | | | | | |
| O75037-3 | Isoform 3 of Kinesin-like protein KIF21B OS = Homo sapiens OX = 9606 | 0 | 0 | 0 | 0.0 | 0.0 |
| | GN = KIF21B - [KI21B_HUMAN] | | | | | |
| O75367-2 | Isoform 1 of Core histone macro-H2A.1 OS = Homo sapiens OX = 9606 | 0 | 0 | 0 | 0.0 | 0.0 |
| | GN = H2AFY - [H2AY_HUMAN] | | | | | |
| O75934 | Pre-mRNA-splicing factor SPF27 OS = Homo sapiens OX = 9606 GN = BCAS2 | 0 | 0 | 0 | 0.0 | 0.0 |
| | PE = 1 SV = 1 - [SPF27_HUMAN] | | | | | |
| O75962-5 | Isoform 5 of Triple functional domain protein OS = Homo sapiens | 0 | 0 | 0 | 0.0 | 0.0 |
| | OX = 9606 GN = TRIO - [TRIO_HUMAN] | | | | | |
| O95551 | Tyrosyl-DNA phosphodiesterase 2 OS = Homo sapiens OX = 9606 GN = TDP2 | 0 | 0 | 0 | 0.0 | 0.0 |
| | PE = 1 SV = 1 - [TYDP2_HUMAN] | | | | | |
| O95639-3 | Isoform 3 of Cleavage and polyadenylation specificity factor subunit 4 | 0 | 0 | 0 | 0.0 | 0.0 |
| | OS = Homo sapiens OX = 9606 GN = CPSF4 - [CPSF4_HUMAN] | | | | | |
| O95786-2 | Isoform 2 of Probable ATP-dependent RNA helicase DDX58 OS = Homo sapiens | 0 | 0 | 0 | 0.0 | 0.0 |
| | OX = 9606 GN = DDX58 - [DDX58_HUMAN] | | | | | |
| O96005-3 | Isoform 2 of Cleft lip and palate transmembrane protein 1 OS = | 0 | 0 | 0 | 0.0 | 0.0 |
| | Homo sapiens OX = 9606 GN = CLPTM1 - [CLPT1_HUMAN] | | | | | |
| P05386 | 60S acidic ribosomal protein P1 OS = Homo sapiens OX = 9606 GN = RPLP1 | 0 | 0 | 0 | 0.0 | 0.0 |
| | PE = 1 SV = 1 - [RLA1_HUMAN] | | | | | |
| P06702 | Protein S100-A9 OS = Homo sapiens OX = 9606 GN = S100A9 PE = 1 SV = | 0 | 0 | 0 | 0.0 | 0.0 |
| | 1 - [S10A9_HUMAN] | | | | | |
| P09914-2 | Isoform 2 of Interferon-induced protein with tetratricopeptide repeats 1 | 0 | 0 | 0 | 0.0 | 0.0 |
| | OS = Homo sapiens OX = 9606 GN = IFIT1 - [IFIT1_HUMAN] | | | | | |
| P11388 | DNA topoisomerase 2-alpha OS = Homo sapiens OX = 9606 GN = TOP2A PE = 1 | 0 | 0 | 0 | 0.0 | 0.0 |
| | SV = 3 - [TOP2A_HUMAN] | | | | | |
| P13807-2 | Isoform 2 of Glycogen [starch] synthase, muscle OS = Homo sapiens | 0 | 0 | 0 | 0.0 | 0.0 |
| | OX = 9606 GN = GYS1 - [GYS1_HUMAN] | | | | | |
| P14678-2 | Isoform SM-B of Small nuclear ribonucleoprotein-associated proteins B and | 0 | 0 | 0 | 0.0 | 0.0 |
| | B′ OS = Homo sapiens OX = 9606 GN = SNRPB - [RSMB_HUMAN] | | | | | |
| P17096 | High mobility group protein HMG-I/HMG-Y OS = Homo sapiens OX = 9606 GN = | 0 | 0 | 0 | 0.0 | 0.0 |
| | HMGA1 PE = 1 SV = 3 - [HMGA1_HUMAN] | | | | | |
| P17096-2 | Isoform HMG-Y of High mobility group protein HMG-I/HMG-Y OS = | 0 | 0 | 0 | 0.0 | 0.0 |
| | Homo sapiens OX = 9606 GN = HMGA1 - [HMGA1_HUMAN] | | | | | |
| P19474 | E3 ubiquitin-protein ligase TRIM21 OS = Homo sapiens OX = 9606 GN = | 0 | 0 | 0 | 0.0 | 0.0 |
| | TRIM21 PE = 1 SV = 1 - [RO52_HUMAN] | | | | | |
| P20592 | Interferon-induced GTP-binding protein Mx2 OS = Homo sapiens OX = | 0 | 0 | 0 | 0.0 | 0.0 |
| | 9606 GN = MX2 PE = 1 SV = 1 - [MX2_HUMAN] | | | | | |
| P22570-4 | Isoform 4 of NADPH: adrenodoxin oxidoreductase, mitochondrial OS = | 0 | 0 | 0 | 0.0 | 0.0 |
| | Homo sapiens OX = 9606 GN = FDXR - [ADRO_HUMAN] | | | | | |
| P22670 | MHC class Il regulatory factor RFX1 OS = Homo sapiens OX = 9606 | 0 | 0 | 0 | 0.0 | 0.0 |
| | GN = RFX1 PE = 1 SV = 2 - [RFX1_HUMAN] | | | | | |
| P22736 | Nuclear receptor subfamily 4 group A member 1 OS = Homo sapiens | 0 | 0 | 0 | 0.0 | 0.0 |
| | OX = 9606 GN = NR4A1 PE = 1 SV = 1 - [NR4A1_HUMAN] | | | | | |
| P23284 | Peptidyl-prolyl cis-trans isomerase B OS = Homo sapiens OX = 9606 | 0 | 0 | 0 | 0.0 | 0.0 |
| | GN = PPIB PE = 1 SV = 2 - [PPIB_HUMAN] | | | | | |
| P25098 | Beta-adrenergic receptor kinase 1 OS = Homo sapiens OX = 9606 | 0 | 0 | 0 | 0.0 | 0.0 |
| | GN = GRK2 PE = 1 SV = 2 - [ARBK1_HUMAN] | | | | | |
| P29728-2 | Isoform p69 of 2′-5′-oligoadenylate synthase 2 OS = Homo sapiens | 0 | 0 | 0 | 0.0 | 0.0 |
| | OX = 9606 GN = OAS2 - [OAS2_HUMAN] | | | | | |
| P31749-2 | Isoform 2 of RAC-alpha serine/threonine-protein kinase OS = Homo sapiens | 0 | 0 | 0 | 0.0 | 0.0 |
| | OX = 9606 GN = AKT1 - [AKT1_HUMAN] | | | | | |
| P32455 | Guanylate-binding protein 1 OS = Homo sapiens OX = 9606 GN = GBP1 PE = 1 | 0 | 0 | 0 | 0.0 | 0.0 |
| | SV = 2 - [GBP1_HUMAN] | | | | | |
| P32456 | Guanylate-binding protein 2 OS = Homo sapiens OX = 9606 GN = GBP2 PE = 1 | 0 | 0 | 0 | 0.0 | 0.0 |
| | SV = 3 - [GBP2_HUMAN] | | | | | |
| P39748-2 | Isoform FENMIT of Flap endonuclease 1 OS = Homo sapiens OX = 9606 | 0 | 0 | 0 | 0.0 | 0.0 |
| | GN = FEN1 - [FEN1_HUMAN] | | | | | |
| P42025 | Beta-centractin OS = Homo sapiens OX = 9606 GN = ACTR1B PE = 1 | 0 | 0 | 0 | 0.0 | 0.0 |
| | SV = 1 - [ACTY_HUMAN] | | | | | |
| P42696-2 | Isoform 2 of RNA-binding protein 34 OS = Homo sapiens OX = 9606 GN = | 0 | 0 | 0 | 0.0 | 0.0 |
| | RBM34 - [RBM34_HUMAN] | | | | | |
| P46060 | Ran GTPase-activating protein 1 OS = Homo sapiens OX = 9606 GN = RANGAP1 | 0 | 0 | 0 | 0.0 | 0.0 |
| | PE = 1 SV = 1 - [RAGP1_HUMAN] | | | | | |
| P48730-2 | Isoform 2 of Casein kinase I isoform delta OS = Homo sapiens OX = 9606 | 0 | 0 | 0 | 0.0 | 0.0 |
| | GN = CSNK1D - [KC1D_HUMAN] | | | | | |
| P50416-2 | Isoform 2 of Carnitine O-palmitoyltransferase 1, liver isoform OS = | 0 | 0 | 0 | 0.0 | 0.0 |
| | Homo sapiens OX = 9606 GN = CPT1A - [CPT1A_HUMAN] | | | | | |
| P50851-2 | Isoform 2 of Lipopolysaccharide-responsive and beige-like anchor protein OS = | 0 | 0 | 0 | 0.0 | 0.0 |
| | Homo sapiens OX = 9606 GN = LRBA - [LRBA_HUMAN] | | | | | |
| P52630-4 | Isoform 2 of Signal transducer and activator of transcription 2 OS = | 0 | 0 | 0 | 0.0 | 0.0 |
| | Homo sapiens OX = 9606 GN = STAT2 - [STAT2_HUMAN] | | | | | |
| P56524 | Histone deacetylase 4 OS = Homo sapiens OX = 9606 GN = HDAC4 PE = 1 | 0 | 0 | 0 | 0.0 | 0.0 |
| | SV = 3 - [HDAC4_HUMAN] | | | | | |
| P63167 | Dynein light chain 1, cytoplasmic OS = Homo sapiens OX = 9606 GN = DYNLL1 | 0 | 0 | 0 | 0.0 | 0.0 |
| | PE = 1 SV = 1 - [DYL1_HUMAN] | | | | | |
| P69905 | CRAP HBA_HUMAN Hemoglobin subunit alpha OS = Homo sapiens GN = HBA1 | 0 | 0 | 0 | 0.0 | 0.0 |
| | PE = 1 SV = 2 | | | | | |
| P85037 | Forkhead box protein K1 OS = Homo sapiens OX = 9606 GN = FOXK1 PE = 1 | 0 | 0 | 0 | 0.0 | 0.0 |
| | SV = 1 - [FOXK1_HUMAN] | | | | | |
| Q01167 | Forkhead box protein K2 OS = Homo sapiens OX = 9606 GN = FOXK2 PE = 1 | 0 | 0 | 0 | 0.0 | 0.0 |
| | SV = 3 - [FOXK2_HUMAN] | | | | | |
| Q01844-6 | Isoform 6 of RNA-binding protein EWS OS = Homo sapiens OX = 9606 | 0 | 0 | 0 | 0.0 | 0.0 |
| | GN = EWSR1 - [EWS_HUMAN] | | | | | |
| Q02880-2 | Isoform Beta-1 of DNA topoisomerase 2-beta OS = Homo sapiens OX = 9606 | 0 | 0 | 0 | 0.0 | 0.0 |
| | GN = TOP2B - [TOP2B_HUMAN] | | | | | |
| Q05086-2 | Isoform I of Ubiquitin-protein ligase E3A OS = Homo sapiens OX = 9606 | 0 | 0 | 0 | 0.0 | 0.0 |
| | GN = UBE3A - [UBE3A_HUMAN] | | | | | |
| Q05519-2 | Isoform 2 of Serine/arginine-rich splicing factor 11 OS = Homo sapiens | 0 | 0 | 0 | 0.0 | 0.0 |
| | OX = 9606 GN = SRSF11 - [SRS11_HUMAN] | | | | | |
| Q05655 | Protein kinase C delta type OS = Homo sapiens OX = 9606 GN = PRKCD | 0 | 0 | 0 | 0.0 | 0.0 |
| | PE = 1 SV = 2 - [KPCD_HUMAN] | | | | | |
| Q07021 | Complement component 1 Q subcomponent-binding protein, mitochondrial OS = | 0 | 0 | 0 | 0.0 | 0.0 |
| | [Homo sapiens OX = 9606 GN = C1QBP PE = 1 SV = 1 - 1QBP_HUMAN] | | | | | |
| Q07666-3 | Isoform 3 of KH domain-containing, RNA-binding, signal transduction- | 0 | 0 | 0 | 0.0 | 0.0 |
| | associated protein 1 OS = Homo sapiens OX = 9606 GN = KHDRBS1 - [KHDR1_HUMAN] | | | | | |
| Q10589-2 | Isoform 2 of Bone marrow stromal antigen 2 OS = Homo sapiens OX = 9606 | 0 | 0 | 0 | 0.0 | 0.0 |
| | GN = BST2 - [BST2_HUMAN] | | | | | |
| Q12888 | TP53-binding protein 1 OS = Homo sapiens OX = 9606 GN = TP53BP1 PE = 1 | 0 | 0 | 0 | 0.0 | 0.0 |
| | SV = 2 - [TP53B_HUMAN] | | | | | |
| Q12965 | Unconventional myosin-le OS = Homo sapiens OX = 9606 GN = MYO1E PE = 1 | 0 | 0 | 0 | 0.0 | 0.0 |
| | SV = 2 - [MYO1E_HUMAN] | | | | | |
| Q13107-2 | Isoform 2 of Ubiquitin carboxyl-terminal hydrolase 4 OS = Homo sapiens | 0 | 0 | 0 | 0.0 | 0.0 |
| | OX = 9606 GN = USP4 - [UBP4_HUMAN] | | | | | |
| Q13136-2 | Isoform 2 of Liprin-alpha-1 OS = Homo sapiens OX = 9606 GN = | 0 | 0 | 0 | 0.0 | 0.0 |
| | PPFIA1 - [LIPA1_HUMAN] | | | | | |
| Q13185 | Chromobox protein homolog 3 OS = Homo sapiens OX = 9606 GN = CBX3 | 0 | 0 | 0 | 0.0 | 0.0 |
| | PE = 1 SV = 4 - [CBX3_HUMAN] | | | | | |
| Q13242 | Serine/arginine-rich splicing factor 9 OS = Homo sapiens OX = 9606 | 0 | 0 | 0 | 0.0 | 0.0 |
| | GN = SRSF9 PE = 1 SV = 1 - [SRSF9_HUMAN] | | | | | |
| Q13325 | Interferon-induced protein with tetratricopeptide repeats 5 OS = | 0 | 0 | 0 | 0.0 | 0.0 |
| | Homo sapiens OX = 9606 GN = IFIT5 PE = 1 SV = 1 - [IFIT5_HUMAN] | | | | | |
| Q13370-2 | Isoform 2 of cGMP-inhibited 3′,5′-cyclic phosphodiesterase B OS = | 0 | 0 | 0 | 0.0 | 0.0 |
| | Homo sapiens OX = 9606 GN = PDE3B - [PDE3B_HUMAN] | | | | | |
| Q13616 | Cullin-1 OS = Homo sapiens OX = 9606 GN = CUL1 PE = 1 SV = 2 - [CUL1_HUMAN] | 0 | 0 | 0 | 0.0 | 0.0 |
| Q13671-2 | Isoform RIN1-delta of Ras and Rab interactor 1 OS = Homo sapiens | 0 | 0 | 0 | 0.0 | 0.0 |
| | OX = 9606 GN = RIN1 - [RIN1_HUMAN] | | | | | |
| Q14254 | Flotillin-2 OS = Homo sapiens OX = 9606 GN = FLOT2 PE = 1 | 0 | 0 | 0 | 0.0 | 0.0 |
| | SV = 2 - [FLOT2_HUMAN] | | | | | |
| Q15075 | Early endosome antigen 1 OS = Homo sapiens OX = 9606 GN = EEA1 PE = 1 | 0 | 0 | 0 | 0.0 | 0.0 |
| | SV = 2 - [EEA1_HUMAN] | | | | | |
| Q15287-3 | Isoform 3 of RNA-binding protein with serine-rich domain 1 OS = | 0 | 0 | 0 | 0.0 | 0.0 |
| | Homo sapiens OX = 9606 GN = RNPS1 - [RNPS1_HUMAN] | | | | | |
| Q15291-2 | Isoform 2 of Retinoblastoma-binding protein 5 OS = Homo sapiens | 0 | 0 | 0 | 0.0 | 0.0 |
| | OX = 9606 GN = RBBP5 - [RBBP5_HUMAN] | | | | | |
| Q15646-3 | Isoform 3 of 2′-5′-oligoadenylate synthase-like protein OS = | 0 | 0 | 0 | 0.0 | 0.0 |
| | Homo sapiens OX = 9606 GN = OASL - [OASL_HUMAN] | | | | | |
| Q15652-2 | Isoform 2 of Probable JmjC domain-containing histone demethylation | 0 | 0 | 0 | 0.0 | 0.0 |
| | protein 2C OS = Homo sapiens OX = 9606 GN = JMJD1C - [JHD2C_HUMAN] | | | | | |
| Q15750-2 | Isoform 2 of TGF-beta-activated kinase 1 and MAP3K7-binding | 0 | 0 | 0 | 0.0 | 0.0 |
| | protein 1 OS = Homo sapiens OX = 9606 GN = TAB1 - [TAB1_HUMAN] | | | | | |
| Q15776-2 | Isoform 2 of Zinc finger protein with KRAB and SCAN domains 8 | 0 | 0 | 0 | 0.0 | 0.0 |
| | OS = Homo sapiens OX = 9606 GN = ZKSCAN8 - [ZKSC8_HUMAN] | | | | | |
| Q32P41 | tRNA (guanine(37)-N1)-methyltransferase OS = Homo sapiens | 0 | 0 | 0 | 0.0 | 0.0 |
| | OX = 9606 GN = TRMT5 PE = 1 SV = 2 - [TRM5_HUMAN] | | | | | |
| Q3SY69 | Mitochondrial 10-formyltetrahydrofolate dehydrogenase OS = | 0 | 0 | 0 | 0.0 | 0.0 |
| | Homo sapiens OX = 9606 GN = ALDH1L2 PE = 1 SV = 2 - [AL1L2_HUMAN] | | | | | |
| Q460N5-4 | Isoform 4 of Protein mono-ADP-ribosyltransferase PARP14 OS = | 0 | 0 | 0 | 0.0 | 0.0 |
| | Homo sapiens OX = 9606 GN = PARP14 - [PAR14_HUMAN] | | | | | |
| Q53G44 | Interferon-induced protein 44-like OS = Homo sapiens OX = 9606 | 0 | 0 | 0 | 0.0 | 0.0 |
| | GN = IFI44L PE = 2 SV = 3 - [IF44L_HUMAN] | | | | | |
| Q5F1R6 | DnaJ homolog subfamily C member 21 OS = Homo sapiens OX = 9606 | 0 | 0 | 0 | 0.0 | 0.0 |
| | GN = DNAJC21 PE = 1 SV = 2 - [DJC21_HUMAN] | | | | | |
| Q5JTC6 | APC membrane recruitment protein 1 OS = Homo sapiens OX = 9606 | 0 | 0 | 0 | 0.0 | 0.0 |
| | GN = AMER1 PE = 1 SV = 2 - [AMER1_HUMAN] | | | | | |
| Q5K651 | Sterile alpha motif domain-containing protein 9 OS = Homo sapiens | 0 | 0 | 0 | 0.0 | 0.0 |
| | OX = 9606 GN = SAMD9 PE = 1 SV = 1 - [SAMD9_HUMAN] | | | | | |
| Q5MNZ6 | WD repeat domain phosphoinositide-interacting protein 3 OS = | 0 | 0 | 0 | 0.0 | 0.0 |
| | Homo sapiens OX = 9606 GN = WDR45B PE = 1 SV = 2 - [WIPI3_HUMAN] | | | | | |
| Q5QJE6 | Deoxynucleotidyltransferase terminal-interacting protein 2 OS = | 0 | 0 | 0 | 0.0 | 0.0 |
| | Homo sapiens OX = 9606 GN = DNTTIP2 PE = 1 SV = 2 - [TDIF2_HUMAN] | | | | | |
| Q5R3I4 | Tetratricopeptide repeat protein 38 OS = Homo sapiens OX = 9606 | 0 | 0 | 0 | 0.0 | 0.0 |
| | GN = TTC38 PE = 1 SV = 1 - [TTC38_HUMAN] | | | | | |
| Q5T749 | Keratinocyte proline-rich protein OS = Homo sapiens OX = 9606 | 0 | 0 | 0 | 0.0 | 0.0 |
| | GN = KPRP PE = 1 SV = 1 - [KPRP_HUMAN] | | | | | |
| Q5TA45-2 | Isoform 2 of Integrator complex subunit 11 OS = Homo sapiens | 0 | 0 | 0 | 0.0 | 0.0 |
| | OX = 9606 GN = INTS11 - [INT11_HUMAN] | | | | | |
| Q68DQ2-1 | Isoform 1 of Very large A-kinase anchor protein OS = Homo sapiens | 0 | 0 | 0 | 0.0 | 0.0 |
| | OX = 9606 GN = CRYBG3 - [CRBG3_HUMAN] | | | | | |
| Q69YN2-2 | Isoform 2 of CWF19-like protein 1 OS = Homo sapiens OX = 9606 | 0 | 0 | 0 | 0.0 | 0.0 |
| | GN = CWF19L1 - [C19L1_HUMAN] | | | | | |
| Q6P1M0 | Long-chain fatty acid transport protein 4 OS = Homo sapiens OX = | 0 | 0 | 0 | 0.0 | 0.0 |
| | 9606 GN = SLC27A4 PE = 1 SV = 1 - [S27A4_HUMAN] | | | | | |
| Q6P3W7 | SCY1-like protein 2 OS = Homo sapiens OX = 9606 GN = SCYL2 | 0 | 0 | 0 | 0.0 | 0.0 |
| | PE = 1 SV = 1 - [SCYL2_HUMAN] | | | | | |
| Q6P3X3 | Tetratricopeptide repeat protein 27 OS = Homo sapiens OX = 9606 | 0 | 0 | 0 | 0.0 | 0.0 |
| | GN = TTC27 PE = 1 SV = 1 - [TTC27_HUMAN] | | | | | |
| Q6P5R6 | 60S ribosomal protein L22-like 1 OS = Homo sapiens OX = 9606 GN = | 0 | 0 | 0 | 0.0 | 0.0 |
| | RPL22L1 PE = 1 SV = 2 - [RL22L_HUMAN] | | | | | |
| Q6PCE3 | Glucose 1,6-bisphosphate synthase OS = Homo sapiens OX = 9606 GN = | 0 | 0 | 0 | 0.0 | 0.0 |
| | PGM2L1 PE = 1 SV = 3 - [PGM2L_HUMAN] | | | | | |
| Q6PIW4-2 | Isoform 2 of Fidgetin-like protein 1 OS = Homo sapiens OX = 9606 | 0 | 0 | 0 | 0.0 | 0.0 |
| | GN = FIGNL1 - [FIGL1_HUMAN] | | | | | |
| Q6Q0C0-2 | Isoform 2 of E3 ubiquitin-protein ligase TRAF7 OS = Homo sapiens | 0 | 0 | 0 | 0.0 | 0.0 |
| | OX = 9606 GN = TRAF7 - [TRAF7_HUMAN] | | | | | |
| Q6R327 | Rapamycin-insensitive companion of mTOR OS = Homo sapiens OX = 9606 | 0 | 0 | 0 | 0.0 | 0.0 |
| | GN = RICTOR PE = 1 SV = 1 - [RICTR_HUMAN] | | | | | |
| Q6T4R5-2 | Isoform 2 of Nance-Horan syndrome protein OS = Homo sapiens | 0 | 0 | 0 | 0.0 | 0.0 |
| | OX = 9606 GN = NHS - [NHS_HUMAN] | | | | | |
| Q6WKZ4-3 | Isoform 2 of Rab11 family-interacting protein 1 OS = Homo sapiens | 0 | 0 | 0 | 0.0 | 0.0 |
| | OX = 9606 GN = RAB11FIP1 - [RFIP1_HUMAN] | | | | | |
| Q6ZSS7 | Major facilitator superfamily domain-containing protein 6 OS = | 0 | 0 | 0 | 0.0 | 0.0 |
| | Homo sapiens OX = 9606 GN = MFSD6 PE = 1 SV = 2 - [MFSD6_HUMAN] | | | | | |
| Q75N03-2 | Isoform 2 of E3 ubiquitin-protein ligase Hakai OS = Homo sapiens | 0 | 0 | 0 | 0.0 | 0.0 |
| | OX = 9606 GN = CBLL1 - [HAKAI_HUMAN] | | | | | |
| Q7Z4S6-6 | Isoform 6 of Kinesin-like protein KIF21A OS = Homo sapiens OX = 9606 | 0 | 0 | 0 | 0.0 | 0.0 |
| | GN = KIF21A - [KI21A_HUMAN] | | | | | |
| Q7Z7A3 | Cytoplasmic tRNA 2-thiolation protein 1 OS = Homo sapiens OX = 9606 | 0 | 0 | 0 | 0.0 | 0.0 |
| | GN = CTU1 PE = 1 SV = 1 - [CTU1_HUMAN] | | | | | |
| Q86TI2-4 | Isoform 3 of Dipeptidyl peptidase 9 OS = Homo sapiens OX = 9606 | 0 | 0 | 0 | 0.0 | 0.0 |
| | GN = DPP9 - [DPP9_HUMAN] | | | | | |
| Q86UE4 | Protein LYRIC OS = Homo sapiens OX = 9606 GN = MTDH PE = 1 | 0 | 0 | 0 | 0.0 | 0.0 |
| | SV = 2 - [LYRIC_HUMAN] | | | | | |
| Q86V21-2 | Isoform 2 of Acetoacetyl-CoA synthetase OS = Homo sapiens OX = 9606 | 0 | 0 | 0 | 0.0 | 0.0 |
| | GN = AACS - [AACS_HUMAN] | | | | | |
| Q86W50 | RNA N6-adenosine-methyltransferase METTL16 OS = Homo sapiens OX = 9606 | 0 | 0 | 0 | 0.0 | 0.0 |
| | GN = METTL16 PE = 1 SV = 2 - [MET16_HUMAN] | | | | | |
| Q86XZ4 | Spermatogenesis-associated serine-rich protein 2 OS = Homo sapiens | 0 | 0 | 0 | 0.0 | 0.0 |
| | OX = 9606 GN = SPATS2 PE = 1 SV = 1 - [SPAS2_HUMAN] | | | | | |
| Q86Y91-6 | Isoform 2 of Kinesin-like protein KIF18B OS = Homo sapiens OX = 9606 | 0 | 0 | 0 | 0.0 | 0.0 |
| | GN = KIF18B - [KI18B_HUMAN] | | | | | |
| Q86YQ8 | Copine-8 OS = Homo sapiens OX = 9606 GN = CPNE8 PE = 1 SV = 2 - [CPNE8_HUMAN] | 0 | 0 | 0 | 0.0 | 0.0 |
| Q8IUE6 | Histone H2A type 2-B OS = Homo sapiens OX = 9606 GN = HIST2H2AB PE = 1 | 0 | 0 | 0 | 0.0 | 0.0 |
| | SV = 3 - [H2A2B_HUMAN] | | | | | |
| Q8IVT5-2 | Isoform 2 of Kinase suppressor of Ras 1 OS = Homo sapiens OX = 9606 | 0 | 0 | 0 | 0.0 | 0.0 |
| | GN = KSR1 - [KSR1_HUMAN] | | | | | |
| Q8IWB1 | Inositol 1,4,5-trisphosphate receptor-interacting protein OS = Homo sapiens | 0 | 0 | 0 | 0.0 | 0.0 |
| | OX = 9606 GN = ITPRIP PE = 1 SV = 1 - [IPRI_HUMAN] | | | | | |
| Q8IY21 | Probable ATP-dependent RNA helicase DDX60 OS = Homo sapiens OX = 9606 | 0 | 0 | 0 | 0.0 | 0.0 |
| | GN = DDX60 PE = 1 SV = 3 - [DDX60_HUMAN] | | | | | |
| Q8N490-2 | Isoform 2 of Probable hydrolase PNKD OS = Homo sapiens OX = 9606 GN = | 0 | 0 | 0 | 0.0 | 0.0 |
| | PNKD - [PNKD_HUMAN] | | | | | |
| Q8N6R0-3 | Isoform 3 of eEF1A lysine and N-terminal methyltransferase OS = Homo sapiens | 0 | 0 | 0 | 0.0 | 0.0 |
| | OX = 9606 GN = EEF1AKNMT - [EFNMT_HUMAN] | | | | | |
| Q8N8S7-3 | Isoform 3 of Protein enabled homolog OS = Homo sapiens OX = 9606 | 0 | 0 | 0 | 0.0 | 0.0 |
| | GN = ENAH - [ENAH_HUMAN] | | | | | |
| Q8N9N7 | Leucine-rich repeat-containing protein 57 OS = Homo sapiens OX = 9606 | 0 | 0 | 0 | 0.0 | 0.0 |
| | GN = LRRC57 PE = 1 SV = 1 - [LRC57_HUMAN] | | | | | |
| Q8NB16-2 | Isoform 2 of Mixed lineage kinase domain-like protein OS = Homo sapiens | 0 | 0 | 0 | 0.0 | 0.0 |
| | OX = 9606 GN = MLKL - [MLKL_HUMAN] | | | | | |
| Q8NBJ5 | Procollagen galactosyltransferase 1 OS = Homo sapiens OX = 9606 GN = | 0 | 0 | 0 | 0.0 | 0.0 |
| | COLGALT1 PE = 1 SV = 1 - [GT251_HUMAN] | | | | | |
| Q8NBQ5 | Estradiol 17-beta-dehydrogenase 11 OS = Homo sapiens OX = 9606 GN = | 0 | 0 | 0 | 0.0 | 0.0 |
| | HSD17B11 PE = 1 SV = 3 - [DHB11_HUMAN] | | | | | |
| Q8NBX0 | Saccharopine dehydrogenase-like oxidoreductase OS = Homo sapiens | 0 | 0 | 0 | 0.0 | 0.0 |
| | OX = 9606 GN = SCCPDH PE = 1 SV = 1 - [SCPDL_HUMAN] | | | | | |
| Q8TB52 | F-box only protein 30 OS = Homo sapiens OX = 9606 GN = FBXO30 | 0 | 0 | 0 | 0.0 | 0.0 |
| | PE = 1 SV = 3 - [FBX30_HUMAN] | | | | | |
| Q8TC12 | Retinol dehydrogenase 11 OS = Homo sapiens OX = 9606 GN = RDH11 PE = 1 | 0 | 0 | 0 | 0.0 | 0.0 |
| | SV = 2 - [RDH11_HUMAN] | | | | | |
| Q8TCB0 | Interferon-induced protein 44 OS = Homo sapiens OX = 9606 GN = IFI44 PE = 2 | 0 | 0 | 0 | 0.0 | 0.0 |
| | SV = 2 - [IFI44_HUMAN] | | | | | |
| Q8TCS8 | Polyribonucleotide nucleotidyltransferase 1, mitochondrial OS = | 0 | 0 | 0 | 0.0 | 0.0 |
| | Homo sapiens OX = 9606 GN = PNPT1 PE = 1 SV = 2 - [PNPT1_HUMAN] | | | | | |
| Q8TEW0-9 | Isoform 9 of Partitioning defective 3 homolog OS = Homo sapiens | 0 | 0 | 0 | 0.0 | 0.0 |
| | OX = 9606 GN = PARD3 - [PARD3_HUMAN] | | | | | |
| Q8WUH6 | Transmembrane protein 263 OS = Homo sapiens OX = 9606 GN = TMEM263 | 0 | 0 | 0 | 0.0 | 0.0 |
| | PE = 1 SV = 1 - [TM263_HUMAN] | | | | | |
| Q8WVJ2 | NudC domain-containing protein 2 OS = Homo sapiens OX = 9606 GN = NUDCD2 | 0 | 0 | 0 | 0.0 | 0.0 |
| | PE = 1 SV = 1 - [NUDC2_HUMAN] | | | | | |
| Q8WVM8-2 | Isoform 2 of Sec1 family domain-containing protein 1 OS = Homo sapiens | 0 | 0 | 0 | 0.0 | 0.0 |
| | OX = 9606 GN = SCFD1 - [SCFD1_HUMAN] | | | | | |
| Q8WVY7 | Ubiquitin-like domain-containing CTD phosphatase 1 OS = Homo sapiens | 0 | 0 | 0 | 0.0 | 0.0 |
| | OX = 9606 GN = UBLCP1 PE = 1 SV = 2 - [UBCP1_HUMAN] | | | | | |
| Q8WWQ0 | PH-interacting protein OS = Homo sapiens OX = 9606 GN = PHIP PE = 1 | 0 | 0 | 0 | 0.0 | 0.0 |
| | SV = 2 - [PHIP_HUMAN] | | | | | |
| Q8WXA3-5 | Isoform 4 of RUN and FYVE domain-containing protein 2 OS = Homo sapiens | 0 | 0 | 0 | 0.0 | 0.0 |
| | OX = 9606 GN = RUFY2 - [RUFY2_HUMAN] | | | | | |
| Q8WXX5 | DnaJ homolog subfamily C member 9 OS = Homo sapiens OX = 9606 | 0 | 0 | 0 | 0.0 | 0.0 |
| | GN = DNAJC9 PE = 1 SV = 1 - [DNJC9_HUMAN] | | | | | |
| Q8WYP5 | Protein ELYS OS = Homo sapiens OX = 9606 GN = AHCTF1 PE = 1 | 0 | 0 | 0 | 0.0 | 0.0 |
| | SV = 3 - [ELYS_HUMAN] | | | | | |
| Q92552 | 28S ribosomal protein S27, mitochondrial OS = Homo sapiens OX = 9606 | 0 | 0 | 0 | 0.0 | 0.0 |
| | GN = MRPS27 PE = 1 SV = 3 - [RT27_HUMAN] | | | | | |
| Q92572 | AP-3 complex subunit sigma-1 OS = Homo sapiens OX = 9606 GN = AP3S1 | 0 | 0 | 0 | 0.0 | 0.0 |
| | PE = 1 SV = 1 - [AP3S1_HUMAN] | | | | | |
| Q92626 | Peroxidasin homolog OS = Homo sapiens OX = 9606 GN = PXDN PE = 1 | 0 | 0 | 0 | 0.0 | 0.0 |
| | SV = 2 - [PXDN_HUMAN] | | | | | |
| Q92985-2 | Isoform B of Interferon regulatory factor 7 OS = Homo sapiens | 0 | 0 | 0 | 0.0 | 0.0 |
| | OX = 9606 GN = IRF7 - [IRF7_HUMAN] | | | | | |
| Q969R5 | Lethal(3)malignant brain tumor-like protein 2 OS = Homo sapiens | 0 | 0 | 0 | 0.0 | 0.0 |
| | OX = 9606 GN = L3MBTL2 PE = 1 SV = 1 - [LMBL2_HUMAN] | | | | | |
| Q969X5-2 | Isoform 2 of Endoplasmic reticulum-Golgi intermediate compartment protein 1 | 0 | 0 | 0 | 0.0 | 0.0 |
| | OS = Homo sapiens OX = 9606 GN = ERGIC1 - [ERGI1_HUMAN] | | | | | |
| Q96B26 | Exosome complex component RRP43 OS = Homo sapiens OX = 9606 GN = | 0 | 0 | 0 | 0.0 | 0.0 |
| | EXOSC8 PE = 1 SV = 1 - [EXOS8_HUMAN] | | | | | |
| Q96BM9 | ADP-ribosylation factor-like protein 8A OS = Homo sapiens OX = | 0 | 0 | 0 | 0.0 | 0.0 |
| | 9606 GN = ARL8A PE = 1 SV = 1 - [ARL8A_HUMAN] | | | | | |
| Q96CU9-3 | Isoform 3 of FAD-dependent oxidoreductase domain-containing | 0 | 0 | 0 | 0.0 | 0.0 |
| | protein 1 OS = Homo sapiens OX = 9606 GN = FOXRED1 - [FXRD1_HUMAN] | | | | | |
| Q96D71-2 | Isoform 2 of RalBP1-associated Eps domain-containing protein 1 | 0 | 0 | 0 | 0.0 | 0.0 |
| | OS = Homo sapiens OX = 9606 GN = REPS1 - [REPS1_HUMAN] | | | | | |
| Q96GQ7 | Probable ATP-dependent RNA helicase DDX27 OS = Homo sapiens | 0 | 0 | 0 | 0.0 | 0.0 |
| | OX = 9606 GN = DDX27 PE = 1 SV = 2 - [DDX27_HUMAN] | | | | | |
| Q96H55-3 | Isoform 3 of Unconventional myosin-XIX OS = Homo sapiens | 0 | 0 | 0 | 0.0 | 0.0 |
| | OX = 9606 GN = MYO19 - [MYO19_HUMAN] | | | | | |
| Q96SI9-2 | Isoform 2 of Spermatid perinuclear RNA-binding protein OS = | 0 | 0 | 0 | 0.0 | 0.0 |
| | Homo sapiens OX = 9606 GN = STRBP - [STRBP_HUMAN] | | | | | |
| Q96ST3 | Paired amphipathic helix protein Sin3a OS = Homo sapiens OX = 9606 GN = SIN3A | 0 | 0 | 0 | 0.0 | 0.0 |
| | PE = 1 SV = 2 - [SIN3A_HUMAN] | | | | | |
| Q99590-2 | Isoform 2 of Protein SCAF11 OS = Homo sapiens OX = 9606 | 0 | 0 | 0 | 0.0 | 0.0 |
| | GN = SCAF11 - [SCAFB_HUMAN] | | | | | |
| Q9BSC4-2 | Isoform 2 of Nucleolar protein 10 OS = Homo sapiens | 0 | 0 | 0 | 0.0 | 0.0 |
| | OX = 9606 GN = NOL10 - [NOL10_HUMAN] | | | | | |
| Q9BT22 | Chitobiosyldiphosphodolichol beta-mannosyltransferase OS = | 0 | 0 | 0 | 0.0 | 0.0 |
| | Homo sapiens OX = 9606 GN = ALG1 PE = 1 SV = 2 - [ALG1_HUMAN] | | | | | |
| Q9BWT3-2 | Isoform 2 of Poly(A) polymerase gamma OS = Homo sapiens | 0 | 0 | 0 | 0.0 | 0.0 |
| | OX = 9606 GN = PAPOLG - [PAPOG_HUMAN] | | | | | |
| Q9BXB4 | Oxysterol-binding protein-related protein 11 OS = Homo sapiens OX = 9606 GN = OSBPL11 | 0 | 0 | 0 | 0.0 | 0.0 |
| | PE = 1 SV = 2 - [OSB11_HUMAN] | | | | | |
| Q9BYK8 | Helicase with zinc finger domain 2 OS = Homo sapiens OX = 9606 | 0 | 0 | 0 | 0.0 | 0.0 |
| | GN = HELZ2 PE = 1 SV = 6 - [HELZ2_HUMAN] | | | | | |
| Q9BYX4 | Interferon-induced helicase C domain-containing protein 1 | 0 | 0 | 0 | 0.0 | 0.0 |
| | OS = Homo sapiens OX = 9606 GN = IFIH1 PE = 1 SV = 3 - [IFIH1_HUMAN] | | | | | |
| Q9BZE9-4 | Isoform 4 of Tether containing UBX domain for GLUT4 OS = | 0 | 0 | 0 | 0.0 | 0.0 |
| | Homo sapiens OX = 9606 GN = ASPSCR1 - [ASPC1_HUMAN] | | | | | |
| Q9C0B5-2 | Isoform 2 of Palmitoyltransferase ZDHHC5 OS = Homo sapiens | 0 | 0 | 0 | 0.0 | 0.0 |
| | OX = 9606 GN = ZDHHC5 - [ZDHC5_HUMAN] | | | | | |
| Q9GZU8 | PSME3-interacting protein OS = Homo sapiens OX = 9606 GN = FAM192A | 0 | 0 | 0 | 0.0 | 0.0 |
| | PE = 1 SV = 1 - [PIP30_HUMAN] | | | | | |
| Q9GZY8-4 | Isoform 4 of Mitochondrial fission factor OS = Homo sapiens | 0 | 0 | 0 | 0.0 | 0.0 |
| | OX = 9606 GN = MFF - [MFF_HUMAN] | | | | | |
| Q9H000-2 | Isoform 2 of Probable E3 ubiquitin-protein ligase makorin-2 OS = | 0 | 0 | 0 | 0.0 | 0.0 |
| | Homo sapiens OX = 9606 GN = MKRN2 - [MKRN2_HUMAN] | | | | | |
| Q9H2P9-2 | Isoform 2 of Diphthine methyl ester synthase OS = Homo sapiens | 0 | 0 | 0 | 0.0 | 0.0 |
| | OX = 9606 GN = DPH5 - [DPH5_HUMAN] | | | | | |
| Q9H6F5 | Coiled-coil domain-containing protein 86 OS = Homo sapiens | 0 | 0 | 0 | 0.0 | 0.0 |
| | OX = 9606 GN = CCDC86 PE = 1 SV = 1 - [CCD86_HUMAN] | | | | | |
| Q9H6S1-3 | Isoform 2 of 5-azacytidine-induced protein 2 OS = | 0 | 0 | 0 | 0.0 | 0.0 |
| | Homo sapiens OX = 9606 GN = AZI2 - [AZI2_HUMAN] | | | | | |
| Q9H7U1-2 | Isoform 2 of Serine-rich coiled-coil domain-containing protein 2 | 0 | 0 | 0 | 0.0 | 0.0 |
| | OS = Homo sapiens OX = 9606 GN = CCSER2 - [CCSE2_HUMAN] | | | | | |
| Q9H8Y5 | Ankyrin repeat and zinc finger domain-containing protein 1 | 0 | 0 | 0 | 0.0 | 0.0 |
| | OS = Homo sapiens OX = 9606 GN = ANKZF1 PE = 1 SV = 1 - [ANKZ1_HUMAN] | | | | | |
| Q9H9F9 | Actin-related protein 5 OS = Homo sapiens OX = 9606 GN = ACTR5 | 0 | 0 | 0 | 0.0 | 0.0 |
| | PE = 1 SV = 2 - [ARP5_HUMAN] | | | | | |
| Q9HAU5 | Regulator of nonsense transcripts 2 OS = Homo sapiens OX = 9606 | 0 | 0 | 0 | 0.0 | 0.0 |
| | GN = UPF2 PE = 1 SV = 1 - [RENT2_HUMAN] | | | | | |
| Q9HC35 | Echinoderm microtubule-associated protein-like 4 OS = | 0 | 0 | 0 | 0.0 | 0.0 |
| | Homo sapiens OX = 9606 GN = EML4 PE = 1 SV = 3 - [EMAL4_HUMAN] | | | | | |
| Q9HCD5 | Nuclear receptor coactivator 5 OS = Homo sapiens OX = 9606 | 0 | 0 | 0 | 0.0 | 0.0 |
| | GN = NCOA5 PE = 1 SV = 2 - [NCOA5_HUMAN] | | | | | |
| Q9HCE1 | Helicase MOV-10 OS = Homo sapiens OX = 9606 GN = MOV10 | 0 | 0 | 0 | 0.0 | 0.0 |
| | PE = 1 SV = 2 - [MOV10_HUMAN] | | | | | |
| Q9NQ55-2 | Isoform 2 of Suppressor of SWI4 1 homolog OS = Homo sapiens | 0 | 0 | 0 | 0.0 | 0.0 |
| | OX = 9606 GN = PPAN - [SSF1_HUMAN] | | | | | |
| Q9NTX5-3 | Isoform 3 of Ethylmalonyl-CoA decarboxylase OS = Homo sapiens | 0 | 0 | 0 | 0.0 | 0.0 |
| | OX = 9606 GN = ECHDC1 - [ECHD1_HUMAN] | | | | | |
| Q9NV88-3 | Isoform 3 of Integrator complex subunit 9 OS = Homo sapiens | 0 | 0 | 0 | 0.0 | 0.0 |
| | OX = 9606 GN = INTS9 - [INT9_HUMAN] | | | | | |
| Q9NVH2-4 | Isoform 4 of Integrator complex subunit 7 OS = Homo sapiens | 0 | 0 | 0 | 0.0 | 0.0 |
| | OX = 9606 GN = INTS7 - [INT7_HUMAN] | | | | | |
| Q9NVJ2 | ADP-ribosylation factor-like protein 8B OS = Homo sapiens | 0 | 0 | 0 | 0.0 | 0.0 |
| | OX = 9606 GN = ARL8B PE = 1 SV = 1 - [ARL8B_HUMAN] | | | | | |
| Q9NX20 | 39S ribosomal protein L16, mitochondrial OS = Homo sapiens | 0 | 0 | 0 | 0.0 | 0.0 |
| | OX = 9606 GN = MRPL16 PE = 1 SV = 1 - [RM16_HUMAN] | | | | | |
| Q9P2E3-2 | Isoform 2 of NFX1-type zinc finger-containing protein 1 | 0 | 0 | 0 | 0.0 | 0.0 |
| | OS = Homo sapiens OX = 9606 GN = ZNFX1 - [ZNFX1_HUMAN] | | | | | |
| Q9UI10-3 | Isoform 3 of Translation initiation factor elF-2B subunit | 0 | 0 | 0 | 0.0 | 0.0 |
| | delta OS = Homo sapiens OX = 9606 GN = EIF2B4 - [EI2BD_HUMAN] | | | | | |
| Q9UIF8-4 | Isoform 4 of Bromodomain adjacent to zinc finger domain] | 0 | 0 | 0 | 0.0 | 0.0 |
| | protein 2B OS = Homo sapiens OX = 9606 GN = BAZ2B - [BAZ2B_HUMAN | | | | | |
| Q9UIG0-2 | Isoform 2 of Tyrosine-protein kinase BAZ1B OS = Homo sapiens | 0 | 0 | 0 | 0.0 | 0.0 |
| | OX = 9606 GN = BAZ1B - [BAZ1B_HUMAN] | | | | | |
| Q9UII4 | E3 ISG15 -- protein ligase HERC5 OS = Homo sapiens OX = 9606 | 0 | 0 | 0 | 0.0 | 0.0 |
| | GN = HERC5 PE = 1 SV = 2 - [HERC5_HUMAN] | | | | | |
| Q9UPP1-5 | Isoform 5 of Histone lysine demethylase PHF8 OS = | 0 | 0 | 0 | 0.0 | 0.0 |
| | Homo sapiens OX = 9606 GN = PHF8 - [PHF8_HUMAN] | | | | | |
| Q9UQR1 | Zinc finger protein 148 OS = Homo sapiens OX = 9606 GN = ZNF148 PE = 1 | 0 | 0 | 0 | 0.0 | 0.0 |
| | SV = 2 - [ZN148_HUMAN] | | | | | |
| Q9Y3Z3-4 | Isoform 4 of Deoxynucleoside triphosphate triphosphohydrolase | 0 | 0 | 0 | 0.0 | 0.0 |
| | SAMHD1 OS = Homo sapiens OX = 9606 GN = SAMHD1 - [SAMH1_HUMAN] | | | | | |
| Q9Y4F1 | FERM, ARHGEF and pleckstrin domain-containing protein 1 OS = Homo sapiens OX = 9606 | 0 | 0 | 0 | 0.0 | 0.0 |
| | GN = FARP1 PE = 1 SV = 1 - [FARP1_HUMAN] | | | | | |
| Q9Y4X5 | E3 ubiquitin-protein ligase ARIH1 OS = Homo sapiens OX = 9606 GN = ARIH1 PE = 1 | 0 | 0 | 0 | 0.0 | 0.0 |
| | SV = 2 - [ARI1_HUMAN] | | | | | |
| Q9Y6K5 | 2′-5′-oligoadenylate synthase 3 OS = Homo sapiens OX = 9606 GN = OAS3 PE = 1 | 0 | 0 | 0 | 0.0 | 0.0 |
| | SV = 3 - [OAS3_HUMAN] | | | | | |
| Q9Y6M0-3 | Isoform 3 of Testisin OS = Homo sapiens OX = 9606 GN = PRSS21 - [TEST_HUMAN] | 0 | 0 | 0 | 0.0 | 0.0 |

TABLE 11
SAINT C-Turbo vs. Control
Cterm Bait

| Prey | PreyGene | Spec | SpecSum | AvgSpec | ctrlCounts | AvgP | MaxP | TopoAvgP | TopoMaxP | SaintScore | logOddsScore | FoldChange | BFDR |
|---|---|---|---|---|---|---|---|---|---|---|---|---|---|
| Q9Y4E8 | USP15 | 59\|69\|61 | 189 | 63 | 4\|4\|7 | 1 | 1 | 1 | 1 | 1 | 52.07 | 12.6 | 0 |
| Q63HN8 | RNF213 | 439\|430\|464 | 1333 | 444.33 | 34\|39\|43 | 1 | 1 | 1 | 1 | 1 | 381.15 | 11.49 | 0 |
| Q9NQC7-2 | CYLD | 10\|7\|6 | 23 | 7.67 | 0\|0\|0 | 1 | 1 | 1 | 1 | 1 | 8.18 | 76.67 | 0 |
| P18031 | PTPN1 | 4\|5\|5 | 14 | 4.67 | 0\|0\|0 | 1 | 1 | 1 | 1 | 1 | 4.87 | 46.67 | 0 |
| Q13045-2 | FLII | 4\|4\|5 | 13 | 4.33 | 0\|0\|0 | 0.99 | 1 | 0.99 | 1 | 0.99 | 4.87 | 43.33 | 0 |
| Q9BVL4 | SELENOO | 3\|4\|4 | 11 | 3.67 | 0\|0\|0 | 0.98 | 0.99 | 0.98 | 0.99 | 0.98 | 3.12 | 36.67 | 0.01 |
| O95819-4 | MAP4K4 | 3\|4\|5 | 12 | 4 | 0\|0\|0 | 0.98 | 1 | 0.98 | 1 | 0.98 | 3.12 | 40 | 0 |
| P30837 | ALDH1B1 | 7\|3\|4 | 14 | 4.67 | 0\|0\|0 | 0.98 | 1 | 0.98 | 1 | 0.98 | 3.12 | 46.67 | 0 |
| Q9NZM1-2 | MYOF | 3\|2\|4 | 9 | 3 | 0\|0\|0 | 0.91 | 0.99 | 0.91 | 0.99 | 0.91 | 1.28 | 30 | 0.02 |
| Q9UM82 | SPATA2 | 10\|6\|14 | 30 | 10 | 2\|0\|0 | 0.91 | 1 | 0.91 | 1 | 0.91 | 1.05 | 15 | 0.03 |
| Q9NP81 | SARS2 | 2\|3\|4 | 9 | 3 | 0\|0\|0 | 0.91 | 0.99 | 0.91 | 0.99 | 0.91 | 1.28 | 30 | 0.02 |
| Q96A49 | SYAP1 | 5\|3\|2 | 10 | 3.33 | 0\|0\|0 | 0.91 | 1 | 0.91 | 1 | 0.91 | 1.28 | 33.33 | 0.01 |
| O15269 | SPTLC1 | 3\|2\|3 | 8 | 2.67 | 0\|0\|0 | 0.9 | 0.96 | 0.9 | 0.96 | 0.9 | 1.28 | 26.67 | 0.04 |
| Q17RY0-2 | CPEB4 | 2\|2\|4 | 8 | 2.67 | 0\|0\|0 | 0.85 | 0.99 | 0.85 | 0.99 | 0.85 | 1.28 | 26.67 | 0.04 |
| P46783 | RPS10 | 2\|2\|2 | 6 | 2 | 0\|0\|0 | 0.78 | 0.78 | 0.78 | 0.78 | 0.78 | 1.28 | 20 | 0.05 |
| P22061 | PCMT1 | 2\|2\|2 | 6 | 2 | 0\|0\|0 | 0.78 | 0.78 | 0.78 | 0.78 | 0.78 | 1.28 | 20 | 0.05 |
| O75794 | CDC123 | 2\|2\|2 | 6 | 2 | 0\|0\|0 | 0.78 | 0.78 | 0.78 | 0.78 | 0.78 | 1.28 | 20 | 0.05 |
| Q16850-2 | CYP51A1 | 4\|0\|4 | 8 | 2.67 | 0\|0\|0 | 0.66 | 0.99 | 0.66 | 0.99 | 0.66 | −3.26 | 26.67 | 0.08 |
| Q9UHD2 | TBK1 | 3\|0\|4 | 7 | 2.33 | 0\|0\|0 | 0.65 | 0.99 | 0.65 | 0.99 | 0.65 | −3.26 | 23.33 | 0.09 |
| Q92890 | UFD1 | 4\|0\|3 | 7 | 2.33 | 0\|0\|0 | 0.65 | 0.99 | 0.65 | 0.99 | 0.65 | −3.26 | 23.33 | 0.09 |
| Q8WUF5 | PPP1R13L | 2\|0\|4 | 6 | 2 | 0\|0\|0 | 0.59 | 0.99 | 0.59 | 0.99 | 0.59 | −3.26 | 20 | 0.13 |
| Q08AE8-2 | SPIRE1 | 6\|0\|2 | 8 | 2.67 | 0\|0\|0 | 0.59 | 1 | 0.59 | 1 | 0.59 | −3.26 | 26.67 | 0.12 |
| Q6YP21-3 | KYAT3 | 4\|0\|2 | 6 | 2 | 0\|0\|0 | 0.59 | 0.99 | 0.59 | 0.99 | 0.59 | −3.26 | 20 | 0.13 |
| Q2M218-2 | AAK1 | 4\|0\|2 | 6 | 2 | 0\|0\|0 | 0.59 | 0.99 | 0.59 | 0.99 | 0.59 | −3.26 | 20 | 0.13 |
| P50747 | HLCS | 0\|4\|2 | 6 | 2 | 0\|0\|0 | 0.59 | 0.99 | 0.59 | 0.99 | 0.59 | −3.26 | 20 | 0.13 |
| Q9Y6Y0 | IVNS1ABP | 2\|0\|4 | 6 | 2 | 0\|0\|0 | 0.59 | 0.99 | 0.59 | 0.99 | 0.59 | −3.26 | 20 | 0.13 |
| Q86SQ0-2 | PHLDB2 | 3\|2\|0 | 5 | 1.67 | 0\|0\|0 | 0.58 | 0.96 | 0.58 | 0.96 | 0.58 | −3.26 | 16.67 | 0.18 |
| Q9H267 | VPS33B | 3\|2\|0 | 5 | 1.67 | 0\|0\|0 | 0.58 | 0.96 | 0.58 | 0.96 | 0.58 | −3.26 | 16.67 | 0.18 |
| Q96IR7 | HPDL | 3\|0\|2 | 5 | 1.67 | 0\|0\|0 | 0.58 | 0.96 | 0.58 | 0.96 | 0.58 | −3.26 | 16.67 | 0.18 |
| A6NED2 | RCCD1 | 3\|0\|2 | 5 | 1.67 | 0\|0\|0 | 0.58 | 0.96 | 0.58 | 0.96 | 0.58 | −3.26 | 16.67 | 0.18 |
| P57737-3 | CORO7 | 0\|3\|2 | 5 | 1.67 | 0\|0\|0 | 0.58 | 0.96 | 0.58 | 0.96 | 0.58 | −3.26 | 16.67 | 0.18 |
| Q86YZ3 | HRNR | 2\|3\|0 | 5 | 1.67 | 0\|0\|0 | 0.58 | 0.96 | 0.58 | 0.96 | 0.58 | −3.26 | 16.67 | 0.18 |
| 075477 | ERLIN1 | 3\|2\|0 | 5 | 1.67 | 0\|0\|0 | 0.58 | 0.96 | 0.58 | 0.96 | 0.58 | −3.26 | 16.67 | 0.18 |
| O02750-2 | MAP2K1 | 3\|0\|2 | 5 | 1.67 | 0\|0\|0 | 0.58 | 0.96 | 0.58 | 0.96 | 0.58 | −3.26 | 16.67 | 0.18 |
| O43896 | KIF1C | 2\|3\|0 | 5 | 1.67 | 0\|0\|0 | 0.58 | 0.96 | 0.58 | 0.96 | 0.58 | −3.26 | 16.67 | 0.18 |
| E9PRG8 | C11ORF98 | 3\|0\|2 | 5 | 1.67 | 0\|0\|0 | 0.58 | 0.96 | 0.58 | 0.96 | 0.58 | −3.26 | 16.67 | 0.18 |
| P29373 | CRABP2 | 4\|7\|4 | 15 | 5 | 2\|0\|0 | 0.55 | 0.85 | 0.55 | 0.85 | 0.55 | −0.42 | 7.5 | 0.25 |
| Q9Y2U8 | LEMD3 | 0\|2\|2 | 4 | 1.33 | 0\|0\|0 | 0.52 | 0.78 | 0.52 | 0.78 | 0.52 | −3.26 | 13.33 | 0.26 |
| Q96T60 | PNKP | 2\|2\|0 | 4 | 1.33 | 0\|0\|0 | 0.52 | 0.78 | 0.52 | 0.78 | 0.52 | −3.26 | 13.33 | 0.26 |
| Q99456 | KRT12 | 2\|2\|0 | 4 | 1.33 | 0\|0\|0 | 0.52 | 0.78 | 0.52 | 0.78 | 0.52 | −3.26 | 13.33 | 0.26 |
| Q9GZS1 | POLR1E | 0\|2\|2 | 4 | 1.33 | 0\|0\|0 | 0.52 | 0.78 | 0.52 | 0.78 | 0.52 | −3.26 | 13.33 | 0.26 |
| Q96A35 | MRPL24 | 2\|0\|2 | 4 | 1.33 | 0\|0\|0 | 0.52 | 0.78 | 0.52 | 0.78 | 0.52 | −3.26 | 13.33 | 0.26 |
| P62258-2 | YWHAE | 2\|2\|0 | 4 | 1.33 | 0\|0\|0 | 0.52 | 0.78 | 0.52 | 0.78 | 0.52 | −3.26 | 13.33 | 0.26 |
| Q9NYL2 | MAP3K20 | 0\|2\|2 | 4 | 1.33 | 0\|0\|0 | 0.52 | 0.78 | 0.52 | 0.78 | 0.52 | −3.26 | 13.33 | 0.26 |
| Q53EP0 | FNDC3B | 2\|0\|2 | 4 | 1.33 | 0\|0\|0 | 0.52 | 0.78 | 0.52 | 0.78 | 0.52 | −3.26 | 13.33 | 0.26 |
| P31350 | RRM2 | 0\|2\|2 | 4 | 1.33 | 0\|0\|0 | 0.52 | 0.78 | 0.52 | 0.78 | 0.52 | −3.26 | 13.33 | 0.26 |
| P51114-3 | FXR1 | 2\|0\|2 | 4 | 1.33 | 0\|0\|0 | 0.52 | 0.78 | 0.52 | 0.78 | 0.52 | −3.26 | 13.33 | 0.26 |
| Q6P2H3-2 | CEP85 | 2\|4\|6 | 12 | 4 | 2\|0\|0 | 0.42 | 0.74 | 0.42 | 0.74 | 0.42 | −2.08 | 6 | 0.3 |
| P04259 | KRT6B | 52\|14\|8 | 74 | 24.67 | 14\|15\|17 | 0.33 | 1 | 0.33 | 1 | 0.33 | −46.96 | 1.61 | 0.32 |
| Q9UFC0 | LRWD1 | 0\|0\|4 | 4 | 1.33 | 0\|0\|0 | 0.33 | 0.99 | 0.33 | 0.99 | 0.33 | −3.26 | 13.33 | 0.34 |
| Q9NZJ0 | DTL | 0\|4\|0 | 4 | 1.33 | 0\|0\|0 | 0.33 | 0.99 | 0.33 | 0.99 | 0.33 | −3.26 | 13.33 | 0.34 |
| Q99439-2 | CNN2 | 4\|0\|0 | 4 | 1.33 | 0\|0\|0 | 0.33 | 0.99 | 0.33 | 0.99 | 0.33 | −3.26 | 13.33 | 0.34 |
| Q9H992-2 | 7-Mar | 0\|0\|4 | 4 | 1.33 | 0\|0\|0 | 0.33 | 0.99 | 0.33 | 0.99 | 0.33 | −3.26 | 13.33 | 0.34 |
| Q6N021 | TET2 | 0\|20\|0 | 20 | 6.67 | 0\|3\|0 | 0.33 | 1 | 0.33 | 1 | 0.33 | −5.85 | 6.67 | 0.33 |
| P02538 | KRT6A | 53\|15\|8 | 76 | 25.33 | 15\|15\|16 | 0.33 | 1 | 0.33 | 1 | 0.33 | −46.96 | 1.65 | 0.32 |
| Q92888-2 | ARHGEF1 | 0\|4\|0 | 4 | 1.33 | 0\|0\|0 | 0.33 | 0.99 | 0.33 | 0.99 | 0.33 | −3.26 | 13.33 | 0.34 |
| P08779 | KRT16 | 76\|13\|8 | 97 | 32.33 | 15\|23\|19 | 0.33 | 1 | 0.33 | 1 | 0.33 | −61.65 | 1.7 | 0.31 |
| Q5T7W7 | TSTD2 | 3\|0\|0 | 3 | 1 | 0\|0\|0 | 0.32 | 0.96 | 0.32 | 0.96 | 0.32 | −3.26 | 10 | 0.37 |
| Q6P1Q9 | METTL2B | 0\|3\|0 | 3 | 1 | 0\|0\|0 | 0.32 | 0.96 | 0.32 | 0.96 | 0.32 | −3.26 | 10 | 0.37 |
| O00443 | PIK3C2A | 0\|3\|0 | 3 | 1 | 0\|0\|0 | 0.32 | 0.96 | 0.32 | 0.96 | 0.32 | −3.26 | 10 | 0.37 |
| Q15814 | TBCC | 0\|3\|0 | 3 | 1 | 0\|0\|0 | 0.32 | 0.96 | 0.32 | 0.96 | 0.32 | −3.26 | 10 | 0.37 |
| Q9P2R3 | ANKFY1 | 3\|0\|0 | 3 | 1 | 0\|0\|0 | 0.32 | 0.96 | 0.32 | 0.96 | 0.32 | −3.26 | 10 | 0.37 |
| O95391 | SLU7 | 0\|3\|0 | 3 | 1 | 0\|0\|0 | 0.32 | 0.96 | 0.32 | 0.96 | 0.32 | −3.26 | 10 | 0.37 |
| Q9H1H9-3 | KIF13A | 0\|0\|3 | 3 | 1 | 0\|0\|0 | 0.32 | 0.96 | 0.32 | 0.96 | 0.32 | −3.26 | 10 | 0.37 |
| O60716-21 | CTNND1 | 0\|0\|3 | 3 | 1 | 0\|0\|0 | 0.32 | 0.96 | 0.32 | 0.96 | 0.32 | −3.26 | 10 | 0.37 |
| Q969S9-2 | GFM2 | 0\|0\|3 | 3 | 1 | 0\|0\|0 | 0.32 | 0.96 | 0.32 | 0.96 | 0.32 | −3.26 | 10 | 0.37 |
| P49674 | CSNK1E | 6\|0\|3 | 9 | 3 | 0\|2\|0 | 0.32 | 0.74 | 0.32 | 0.74 | 0.32 | −5.43 | 4.5 | 0.37 |
| Q9H0W8-2 | SMG9 | 0\|0\|3 | 3 | 1 | 0\|0\|0 | 0.32 | 0.96 | 0.32 | 0.96 | 0.32 | −3.26 | 10 | 0.37 |
| Q15070-2 | OXA1L | 0\|3\|0 | 3 | 1 | 0\|0\|0 | 0.32 | 0.96 | 0.32 | 0.96 | 0.32 | −3.26 | 10 | 0.37 |
| Q9BQL6 | FERMT1 | 0\|3\|0 | 3 | 1 | 0\|0\|0 | 0.32 | 0.96 | 0.32 | 0.96 | 0.32 | −3.26 | 10 | 0.37 |
| P18074-2 | ERCC2 | 0\|0\|3 | 3 | 1 | 0\|0\|0 | 0.32 | 0.96 | 0.32 | 0.96 | 0.32 | −3.26 | 10 | 0.37 |
| Q8NB46 | ANKRD52 | 0\|0\|3 | 3 | 1 | 0\|0\|0 | 0.32 | 0.96 | 0.32 | 0.96 | 0.32 | −3.26 | 10 | 0.37 |
| Q9UL54-2 | TAOK2 | 0\|0\|3 | 3 | 1 | 0\|0\|0 | 0.32 | 0.96 | 0.32 | 0.96 | 0.32 | −3.26 | 10 | 0.37 |
| O96013 | PAK4 | 3\|0\|0 | 3 | 1 | 0\|0\|0 | 0.32 | 0.96 | 0.32 | 0.96 | 0.32 | −3.26 | 10 | 0.37 |
| Q9BQ70 | TCF25 | 0\|0\|3 | 3 | 1 | 0\|0\|0 | 0.32 | 0.96 | 0.32 | 0.96 | 0.32 | −3.26 | 10 | 0.37 |
| P60228 | EIF3E | 3\|0\|0 | 3 | 1 | 0\|0\|0 | 0.32 | 0.96 | 0.32 | 0.96 | 0.32 | −3.26 | 10 | 0.37 |
| Q9Y217 | PIKFYVE | 3\|0\|0 | 3 | 1 | 0\|0\|0 | 0.32 | 0.96 | 0.32 | 0.96 | 0.32 | −3.26 | 10 | 0.37 |
| O75592-2 | MYCBP2 | 5\|2\|3 | 10 | 3.33 | 0\|2\|0 | 0.31 | 0.58 | 0.31 | 0.58 | 0.31 | −2.08 | 5 | 0.45 |
| P08240 | SRPRA | 5\|3\|2 | 10 | 3.33 | 2\|0\|0 | 0.31 | 0.58 | 0.31 | 0.58 | 0.31 | −2.08 | 5 | 0.45 |
| P08865 | RPSA | 4\|3\|3 | 10 | 3.33 | 0\|0\|2 | 0.29 | 0.4 | 0.29 | 0.4 | 0.29 | −1.21 | 5 | 0.45 |
| Q8WXG6-6 | MADD | 2\|0\|0 | 2 | 0.67 | 0\|0\|0 | 0.26 | 0.78 | 0.26 | 0.78 | 0.26 | −3.26 | 6.67 | 0.46 |
| Q01433-2 | AMPD2 | 2\|0\|0 | 2 | 0.67 | 0\|0\|0 | 0.26 | 0.78 | 0.26 | 0.78 | 0.26 | −3.26 | 6.67 | 0.46 |
| Q9UKL0 | RCOR1 | 0\|0\|2 | 2 | 0.67 | 0\|0\|0 | 0.26 | 0.78 | 0.26 | 0.78 | 0.26 | −3.26 | 6.67 | 0.46 |
| P49593-2 | PPM1F | 2\|0\|0 | 2 | 0.67 | 0\|0\|0 | 0.26 | 0.78 | 0.26 | 0.78 | 0.26 | −3.26 | 6.67 | 0.46 |
| Q9H8H0 | NOL11 | 0\|2\|0 | 2 | 0.67 | 0\|0\|0 | 0.26 | 0.78 | 0.26 | 0.78 | 0.26 | −3.26 | 6.67 | 0.46 |
| O75955-2 | FLOT1 | 0\|2\|0 | 2 | 0.67 | 0\|0\|0 | 0.26 | 0.78 | 0.26 | 0.78 | 0.26 | −3.26 | 6.67 | 0.46 |
| P41214 | EIF2D | 0\|2\|0 | 2 | 0.67 | 0\|0\|0 | 0.26 | 0.78 | 0.26 | 0.78 | 0.26 | −3.26 | 6.67 | 0.46 |
| A5YKK6 | CNOT1 | 2\|0\|0 | 2 | 0.67 | 0\|0\|0 | 0.26 | 0.78 | 0.26 | 0.78 | 0.26 | −3.26 | 6.67 | 0.46 |
| Q13555-10 | CAMK2G | 0\|0\|2 | 2 | 0.67 | 0\|0\|0 | 0.26 | 0.78 | 0.26 | 0.78 | 0.26 | −3.26 | 6.67 | 0.46 |
| Q05682-5 | CALD1 | 0\|0\|2 | 2 | 0.67 | 0\|0\|0 | 0.26 | 0.78 | 0.26 | 0.78 | 0.26 | −3.26 | 6.67 | 0.46 |
| Q9Y2Z4 | YARS2 | 0\|0\|2 | 2 | 0.67 | 0\|0\|0 | 0.26 | 0.78 | 0.26 | 0.78 | 0.26 | −3.26 | 6.67 | 0.46 |
| P51116 | FXR2 | 0\|2\|0 | 2 | 0.67 | 0\|0\|0 | 0.26 | 0.78 | 0.26 | 0.78 | 0.26 | −3.26 | 6.67 | 0.46 |
| Q9NVC6 | MED17 | 0\|2\|0 | 2 | 0.67 | 0\|0\|0 | 0.26 | 0.78 | 0.26 | 0.78 | 0.26 | −3.26 | 6.67 | 0.46 |
| Q9Y619 | SLC25A15 | 0\|0\|2 | 2 | 0.67 | 0\|0\|0 | 0.26 | 0.78 | 0.26 | 0.78 | 0.26 | −3.26 | 6.67 | 0.46 |
| Q8N726-2 | CDKN2A | 0\|2\|0 | 2 | 0.67 | 0\|0\|0 | 0.26 | 0.78 | 0.26 | 0.78 | 0.26 | −3.26 | 6.67 | 0.46 |
| Q96S44 | TP53RK | 2\|0\|0 | 2 | 0.67 | 0\|0\|0 | 0.26 | 0.78 | 0.26 | 0.78 | 0.26 | −3.26 | 6.67 | 0.46 |
| Q8WWC4 | MAIP1 | 0\|2\|0 | 2 | 0.67 | 0\|0\|0 | 0.26 | 0.78 | 0.26 | 0.78 | 0.26 | −3.26 | 6.67 | 0.46 |
| Q9C0J8 | WDR33 | 0\|0\|2 | 2 | 0.67 | 0\|0\|0 | 0.26 | 0.78 | 0.26 | 0.78 | 0.26 | −3.26 | 6.67 | 0.46 |
| Q15208 | STK38 | 0\|2\|0 | 2 | 0.67 | 0\|0\|0 | 0.26 | 0.78 | 0.26 | 0.78 | 0.26 | −3.26 | 6.67 | 0.46 |
| P78362 | SRPK2 | 0\|2\|0 | 2 | 0.67 | 0\|0\|0 | 0.26 | 0.78 | 0.26 | 0.78 | 0.26 | −3.26 | 6.67 | 0.46 |
| Q96R06 | SPAG5 | 2\|0\|0 | 2 | 0.67 | 0\|0\|0 | 0.26 | 0.78 | 0.26 | 0.78 | 0.26 | −3.26 | 6.67 | 0.46 |
| Q96RE7 | NACC1 | 0\|0\|2 | 2 | 0.67 | 0\|0\|0 | 0.26 | 0.78 | 0.26 | 0.78 | 0.26 | −3.26 | 6.67 | 0.46 |
| O14925 | TIMM23 | 0\|2\|0 | 2 | 0.67 | 0\|0\|0 | 0.26 | 0.78 | 0.26 | 0.78 | 0.26 | −3.26 | 6.67 | 0.46 |
| Q8N1G4 | LRRC47 | 0\|0\|2 | 2 | 0.67 | 0\|0\|0 | 0.26 | 0.78 | 0.26 | 0.78 | 0.26 | −3.26 | 6.67 | 0.46 |
| Q14116-2 | IL18 | 0\|2\|0 | 2 | 0.67 | 0\|0\|0 | 0.26 | 0.78 | 0.26 | 0.78 | 0.26 | −3.26 | 6.67 | 0.46 |
| Q96CB8 | INTS12 | 0\|2\|0 | 2 | 0.67 | 0\|0\|0 | 0.26 | 0.78 | 0.26 | 0.78 | 0.26 | −3.26 | 6.67 | 0.46 |
| O43852-5 | CALU | 0\|0\|2 | 2 | 0.67 | 0\|0\|0 | 0.26 | 0.78 | 0.26 | 0.78 | 0.26 | −3.26 | 6.67 | 0.46 |
| Q15036-2 | SNX17 | 2\|0\|0 | 2 | 0.67 | 0\|0\|0 | 0.26 | 0.78 | 0.26 | 0.78 | 0.26 | −3.26 | 6.67 | 0.46 |
| P50570-2 | DNM2 | 0\|2\|0 | 2 | 0.67 | 0\|0\|0 | 0.26 | 0.78 | 0.26 | 0.78 | 0.26 | −3.26 | 6.67 | 0.46 |
| Q96SU4-5 | OSBPL9 | 0\|0\|2 | 2 | 0.67 | 0\|0\|0 | 0.26 | 0.78 | 0.26 | 0.78 | 0.26 | −3.26 | 6.67 | 0.46 |
| Q9ULV4 | CORO1C | 2\|0\|0 | 2 | 0.67 | 0\|0\|0 | 0.26 | 0.78 | 0.26 | 0.78 | 0.26 | −3.26 | 6.67 | 0.46 |
| Q14106-2 | TOB2 | 0\|0\|2 | 2 | 0.67 | 0\|0\|0 | 0.26 | 0.78 | 0.26 | 0.78 | 0.26 | −3.26 | 6.67 | 0.46 |
| O60869-2 | EDF1 | 0\|0\|2 | 2 | 0.67 | 0\|0\|0 | 0.26 | 0.78 | 0.26 | 0.78 | 0.26 | −3.26 | 6.67 | 0.46 |
| P62857 | RPS28 | 0\|0\|2 | 2 | 0.67 | 0\|0\|0 | 0.26 | 0.78 | 0.26 | 0.78 | 0.26 | −3.26 | 6.67 | 0.46 |
| Q5VZL5-2 | ZMYM4 | 0\|0\|2 | 2 | 0.67 | 0\|0\|0 | 0.26 | 0.78 | 0.26 | 0.78 | 0.26 | −3.26 | 6.67 | 0.46 |
| Q86V81 | ALYREF | 2\|0\|0 | 2 | 0.67 | 0\|0\|0 | 0.26 | 0.78 | 0.26 | 0.78 | 0.26 | −3.26 | 6.67 | 0.46 |
| P02662 | P02662 | 2\|0\|0 | 2 | 0.67 | 0\|0\|0 | 0.26 | 0.78 | 0.26 | 0.78 | 0.26 | −3.26 | 6.67 | 0.46 |
| Q99497 | PARK7 | 0\|0\|2 | 2 | 0.67 | 0\|0\|0 | 0.26 | 0.78 | 0.26 | 0.78 | 0.26 | −3.26 | 6.67 | 0.46 |
| Q8ND56-3 | LSM14A | 2\|0\|0 | 2 | 0.67 | 0\|0\|0 | 0.26 | 0.78 | 0.26 | 0.78 | 0.26 | −3.26 | 6.67 | 0.46 |
| P17252 | PRKCA | 0\|0\|2 | 2 | 0.67 | 0\|0\|0 | 0.26 | 0.78 | 0.26 | 0.78 | 0.26 | −3.26 | 6.67 | 0.46 |
| Q9NX46 | ADPRHL2 | 2\|0\|0 | 2 | 0.67 | 0\|0\|0 | 0.26 | 0.78 | 0.26 | 0.78 | 0.26 | −3.26 | 6.67 | 0.46 |
| Q9BUZ4 | TRAF4 | 2\|0\|0 | 2 | 0.67 | 0\|0\|0 | 0.26 | 0.78 | 0.26 | 0.78 | 0.26 | −3.26 | 6.67 | 0.46 |
| Q15154-4 | PCM1 | 2\|0\|0 | 2 | 0.67 | 0\|0\|0 | 0.26 | 0.78 | 0.26 | 0.78 | 0.26 | −3.26 | 6.67 | 0.46 |
| Q9BUH8 | BEGAIN | 2\|0\|0 | 2 | 0.67 | 0\|0\|0 | 0.26 | 0.78 | 0.26 | 0.78 | 0.26 | −3.26 | 6.67 | 0.46 |
| Q8NF37 | LPCAT1 | 0\|0\|2 | 2 | 0.67 | 0\|0\|0 | 0.26 | 0.78 | 0.26 | 0.78 | 0.26 | −3.26 | 6.67 | 0.46 |
| Q9NZT1 | CALML5 | 2\|0\|0 | 2 | 0.67 | 0\|0\|0 | 0.26 | 0.78 | 0.26 | 0.78 | 0.26 | −3.26 | 6.67 | 0.46 |
| O15198-2 | SMAD9 | 2\|0\|0 | 2 | 0.67 | 0\|0\|0 | 0.26 | 0.78 | 0.26 | 0.78 | 0.26 | −3.26 | 6.67 | 0.46 |
| Q9UJK0 | TSR3 | 0\|2\|0 | 2 | 0.67 | 0\|0\|0 | 0.26 | 0.78 | 0.26 | 0.78 | 0.26 | −3.26 | 6.67 | 0.46 |
| Q9Y2S6 | TMA7 | 0\|0\|2 | 2 | 0.67 | 0\|0\|0 | 0.26 | 0.78 | 0.26 | 0.78 | 0.26 | −3.26 | 6.67 | 0.46 |
| Q9BT92 | TCHP | 0\|0\|2 | 2 | 0.67 | 0\|0\|0 | 0.26 | 0.78 | 0.26 | 0.78 | 0.26 | −3.26 | 6.67 | 0.46 |
| Q7L0Y3 | TRMT10C | 0\|0\|2 | 2 | 0.67 | 0\|0\|0 | 0.26 | 0.78 | 0.26 | 0.78 | 0.26 | −3.26 | 6.67 | 0.46 |
| P45973 | CBX5 | 2\|0\|0 | 2 | 0.67 | 0\|0\|0 | 0.26 | 0.78 | 0.26 | 0.78 | 0.26 | −3.26 | 6.67 | 0.46 |
| Q16881-5 | TXNRD1 | 2\|0\|0 | 2 | 0.67 | 0\|0\|0 | 0.26 | 0.78 | 0.26 | 0.78 | 0.26 | −3.26 | 6.67 | 0.46 |
| Q86VM9 | ZC3H18 | 0\|2\|0 | 2 | 0.67 | 0\|0\|0 | 0.26 | 0.78 | 0.26 | 0.78 | 0.26 | −3.26 | 6.67 | 0.46 |
| Q9H479 | FN3K | 0\|0\|2 | 2 | 0.67 | 0\|0\|0 | 0.26 | 0.78 | 0.26 | 0.78 | 0.26 | −3.26 | 6.67 | 0.46 |
| Q99805 | TM9SF2 | 2\|0\|0 | 2 | 0.67 | 0\|0\|0 | 0.26 | 0.78 | 0.26 | 0.78 | 0.26 | −3.26 | 6.67 | 0.46 |
| Q9UM00-1 | TMCO1 | 0\|2\|0 | 2 | 0.67 | 0\|0\|0 | 0.26 | 0.78 | 0.26 | 0.78 | 0.26 | −3.26 | 6.67 | 0.46 |
| Q13888 | GTF2H2 | 0\|0\|2 | 2 | 0.67 | 0\|0\|0 | 0.26 | 0.78 | 0.26 | 0.78 | 0.26 | −3.26 | 6.67 | 0.46 |
| Q5VYK3 | ECM29 | 0\|2\|0 | 2 | 0.67 | 0\|0\|0 | 0.26 | 0.78 | 0.26 | 0.78 | 0.26 | −3.26 | 6.67 | 0.46 |
| P06789 | P06789 | 2\|0\|0 | 2 | 0.67 | 0\|0\|0 | 0.26 | 0.78 | 0.26 | 0.78 | 0.26 | −3.26 | 6.67 | 0.46 |
| P06788 | P06788 | 0\|0\|2 | 2 | 0.67 | 0\|0\|0 | 0.26 | 0.78 | 0.26 | 0.78 | 0.26 | −3.26 | 6.67 | 0.46 |
| P29966 | MARCKS | 0\|0\|2 | 2 | 0.67 | 0\|0\|0 | 0.26 | 0.78 | 0.26 | 0.78 | 0.26 | −3.26 | 6.67 | 0.46 |
| Q6IN84 | MRM1 | 0\|0\|2 | 2 | 0.67 | 0\|0\|0 | 0.26 | 0.78 | 0.26 | 0.78 | 0.26 | −3.26 | 6.67 | 0.46 |
| Q9P210 | CPSF2 | 0\|2\|0 | 2 | 0.67 | 0\|0\|0 | 0.26 | 0.78 | 0.26 | 0.78 | 0.26 | −3.26 | 6.67 | 0.46 |
| P53814-5 | SMTN | 2\|0\|0 | 2 | 0.67 | 0\|0\|0 | 0.26 | 0.78 | 0.26 | 0.78 | 0.26 | −3.26 | 6.67 | 0.46 |
| Q13332-6 | PTPRS | 0\|0\|2 | 2 | 0.67 | 0\|0\|0 | 0.26 | 0.78 | 0.26 | 0.78 | 0.26 | −3.26 | 6.67 | 0.46 |
| O60256 | PRPSAP2 | 2\|0\|0 | 2 | 0.67 | 0\|0\|0 | 0.26 | 0.78 | 0.26 | 0.78 | 0.26 | −3.26 | 6.67 | 0.46 |
| P78312-4 | FAM193A | 0\|0\|2 | 2 | 0.67 | 0\|0\|0 | 0.26 | 0.78 | 0.26 | 0.78 | 0.26 | −3.26 | 6.67 | 0.46 |
| O43314-2 | PPIP5K2 | 2\|0\|0 | 2 | 0.67 | 0\|0\|0 | 0.26 | 0.78 | 0.26 | 0.78 | 0.26 | −3.26 | 6.67 | 0.46 |
| P38117 | ETFB | 2\|0\|0 | 2 | 0.67 | 0\|0\|0 | 0.26 | 0.78 | 0.26 | 0.78 | 0.26 | −3.26 | 6.67 | 0.46 |
| Q86VP6 | CAND1 | 2\|0\|0 | 2 | 0.67 | 0\|0\|0 | 0.26 | 0.78 | 0.26 | 0.78 | 0.26 | −3.26 | 6.67 | 0.46 |
| Q9NVZ3-4 | NECAP2 | 0\|0\|2 | 2 | 0.67 | 0\|0\|0 | 0.26 | 0.78 | 0.26 | 0.78 | 0.26 | −3.26 | 6.67 | 0.46 |
| Q7Z4G4-3 | TRMT11 | 0\|0\|2 | 2 | 0.67 | 0\|0\|0 | 0.26 | 0.78 | 0.26 | 0.78 | 0.26 | −3.26 | 6.67 | 0.46 |
| Q8WVX9 | FAR1 | 2\|0\|0 | 2 | 0.67 | 0\|0\|0 | 0.26 | 0.78 | 0.26 | 0.78 | 0.26 | −3.26 | 6.67 | 0.46 |
| P21281 | ATP6V1B2 | 2\|0\|0 | 2 | 0.67 | 0\|0\|0 | 0.26 | 0.78 | 0.26 | 0.78 | 0.26 | −3.26 | 6.67 | 0.46 |
| Q7L7X3-3 | TAOK1 | 0\|0\|2 | 2 | 0.67 | 0\|0\|0 | 0.26 | 0.78 | 0.26 | 0.78 | 0.26 | −3.26 | 6.67 | 0.46 |
| Q15058 | KIF14 | 2\|0\|0 | 2 | 0.67 | 0\|0\|0 | 0.26 | 0.78 | 0.26 | 0.78 | 0.26 | −3.26 | 6.67 | 0.46 |
| Q9BTL3 | FAM103A1 | 0\|0\|2 | 2 | 0.67 | 0\|0\|0 | 0.26 | 0.78 | 0.26 | 0.78 | 0.26 | −3.26 | 6.67 | 0.46 |
| Q5VST9-5 | OBSCN | 2\|0\|0 | 2 | 0.67 | 0\|0\|0 | 0.26 | 0.78 | 0.26 | 0.78 | 0.26 | −3.26 | 6.67 | 0.46 |
| P53582 | METAP1 | 0\|2\|0 | 2 | 0.67 | 0\|0\|0 | 0.26 | 0.78 | 0.26 | 0.78 | 0.26 | −3.26 | 6.67 | 0.46 |
| P08574 | CYC1 | 2\|0\|0 | 2 | 0.67 | 0\|0\|0 | 0.26 | 0.78 | 0.26 | 0.78 | 0.26 | −3.26 | 6.67 | 0.46 |
| O75616 | ERAL1 | 0\|0\|2 | 2 | 0.67 | 0\|0\|0 | 0.26 | 0.78 | 0.26 | 0.78 | 0.26 | −3.26 | 6.67 | 0.46 |
| P53680-2 | AP2S1 | 2\|0\|0 | 2 | 0.67 | 0\|0\|0 | 0.26 | 0.78 | 0.26 | 0.78 | 0.26 | −3.26 | 6.67 | 0.46 |
| Q03164-2 | KMT2A | 0\|0\|2 | 2 | 0.67 | 0\|0\|0 | 0.26 | 0.78 | 0.26 | 0.78 | 0.26 | −3.26 | 6.67 | 0.46 |
| Q15418-4 | RPS6KA1 | 2\|0\|0 | 2 | 0.67 | 0\|0\|0 | 0.26 | 0.78 | 0.26 | 0.78 | 0.26 | −3.26 | 6.67 | 0.46 |
| P49642 | PRIM1 | 0\|0\|2 | 2 | 0.67 | 0\|0\|0 | 0.26 | 0.78 | 0.26 | 0.78 | 0.26 | −3.26 | 6.67 | 0.46 |
| Q15437 | SEC23B | 0\|2\|0 | 2 | 0.67 | 0\|0\|0 | 0.26 | 0.78 | 0.26 | 0.78 | 0.26 | −3.26 | 6.67 | 0.46 |
| P16989-2 | YBX3 | 2\|0\|0 | 2 | 0.67 | 0\|0\|0 | 0.26 | 0.78 | 0.26 | 0.78 | 0.26 | −3.26 | 6.67 | 0.46 |
| Q8IZ73-2 | RPUSD2 | 0\|0\|2 | 2 | 0.67 | 0\|0\|0 | 0.26 | 0.78 | 0.26 | 0.78 | 0.26 | −3.26 | 6.67 | 0.46 |
| Q86W56 | PARG | 0\|2\|0 | 2 | 0.67 | 0\|0\|0 | 0.26 | 0.78 | 0.26 | 0.78 | 0.26 | −3.26 | 6.67 | 0.46 |
| P82912-2 | MRPS11 | 0\|0\|2 | 2 | 0.67 | 0\|0\|0 | 0.26 | 0.78 | 0.26 | 0.78 | 0.26 | −3.26 | 6.67 | 0.46 |
| P09429 | HMGB1 | 0\|0\|2 | 2 | 0.67 | 0\|0\|0 | 0.26 | 0.78 | 0.26 | 0.78 | 0.26 | −3.26 | 6.67 | 0.46 |
| Q8TCG1-2 | KIAA1524 | 0\|2\|0 | 2 | 0.67 | 0\|0\|0 | 0.26 | 0.78 | 0.26 | 0.78 | 0.26 | −3.26 | 6.67 | 0.46 |
| O15270 | SPTLC2 | 0\|2\|0 | 2 | 0.67 | 0\|0\|0 | 0.26 | 0.78 | 0.26 | 0.78 | 0.26 | −3.26 | 6.67 | 0.46 |
| Q9NYL2-2 | MAP3K20 | 0\|2\|0 | 2 | 0.67 | 0\|0\|0 | 0.26 | 0.78 | 0.26 | 0.78 | 0.26 | −3.26 | 6.67 | 0.46 |
| Q9BU76-4 | MMTAG2 | 0\|0\|2 | 2 | 0.67 | 0\|0\|0 | 0.26 | 0.78 | 0.26 | 0.78 | 0.26 | −3.26 | 6.67 | 0.46 |
| O43402 | EMC8 | 0\|2\|0 | 2 | 0.67 | 0\|0\|0 | 0.26 | 0.78 | 0.26 | 0.78 | 0.26 | −3.26 | 6.67 | 0.46 |
| Q92538-3 | GBF1 | 0\|0\|2 | 2 | 0.67 | 0\|0\|0 | 0.26 | 0.78 | 0.26 | 0.78 | 0.26 | −3.26 | 6.67 | 0.46 |
| Q6IAN0 | DHRS7B | 2\|0\|0 | 2 | 0.67 | 0\|0\|0 | 0.26 | 0.78 | 0.26 | 0.78 | 0.26 | −3.26 | 6.67 | 0.46 |
| Q86UL3 | GPAT4 | 0\|0\|2 | 2 | 0.67 | 0\|0\|0 | 0.26 | 0.78 | 0.26 | 0.78 | 0.26 | −3.26 | 6.67 | 0.46 |
| Q6P1N0-2 | CC2D1A | 2\|0\|0 | 2 | 0.67 | 0\|0\|0 | 0.26 | 0.78 | 0.26 | 0.78 | 0.26 | −3.26 | 6.67 | 0.46 |
| P35270 | SPR | 0\|2\|0 | 2 | 0.67 | 0\|0\|0 | 0.26 | 0.78 | 0.26 | 0.78 | 0.26 | −3.26 | 6.67 | 0.46 |
| O43294-2 | TGFB111 | 0\|2\|0 | 2 | 0.67 | 0\|0\|0 | 0.26 | 0.78 | 0.26 | 0.78 | 0.26 | −3.26 | 6.67 | 0.46 |
| P60660 | MYL6 | 3\|2\|4 | 9 | 3 | 2\|0\|0 | 0.25 | 0.4 | 0.25 | 0.4 | 0.25 | −2.08 | 4.5 | 0.62 |
| Q5SWA1 | PPP1R15B | 2\|3\|4 | 9 | 3 | 0\|2\|0 | 0.25 | 0.4 | 0.25 | 0.4 | 0.25 | −2.08 | 4.5 | 0.62 |
| Q9H425 | C1ORF198 | 3\|3\|3 | 9 | 3 | 0\|0\|2 | 0.23 | 0.23 | 0.23 | 0.23 | 0.23 | −1.21 | 4.5 | 0.62 |
| P61604 | HSPE1 | 3\|3\|3 | 9 | 3 | 0\|0\|2 | 0.23 | 0.23 | 0.23 | 0.23 | 0.23 | −1.21 | 4.5 | 0.62 |
| O60884 | DNAJA2 | 5\|0\|2 | 7 | 2.33 | 0\|0\|2 | 0.23 | 0.58 | 0.23 | 0.58 | 0.23 | −5.43 | 3.5 | 0.62 |
| A0FGR8-2 | ESYT2 | 0\|4\|3 | 7 | 2.33 | 0\|0\|2 | 0.21 | 0.4 | 0.21 | 0.4 | 0.21 | −5.43 | 3.5 | 0.62 |
| P49589-3 | CARS | 3\|0\|4 | 7 | 2.33 | 2\|0\|0 | 0.21 | 0.4 | 0.21 | 0.4 | 0.21 | −5.43 | 3.5 | 0.62 |
| Q12792-4 | TWF1 | 5\|3\|3 | 11 | 3.67 | 3\|0\|0 | 0.2 | 0.34 | 0.2 | 0.34 | 0.2 | −1.89 | 3.67 | 0.62 |
| P25789 | PSMA4 | 2\|3\|3 | 8 | 2.67 | 0\|0\|2 | 0.19 | 0.23 | 0.19 | 0.23 | 0.19 | −2.08 | 4 | 0.62 |
| P35222 | CTNNB1 | 2\|3\|3 | 8 | 2.67 | 0\|2\|0 | 0.19 | 0.23 | 0.19 | 0.23 | 0.19 | −2.08 | 4 | 0.62 |
| Q9UQ13-2 | SHOC2 | 2\|0\|4 | 6 | 2 | 2\|0\|0 | 0.17 | 0.4 | 0.17 | 0.4 | 0.17 | −5.43 | 3 | 0.62 |
| P43307 | SSR1 | 5\|0\|0 | 5 | 1.67 | 0\|2\|2 | 0.15 | 0.44 | 0.15 | 0.44 | 0.15 | −7.85 | 1.25 | 0.63 |
| Q15018 | ABRAXAS2 | 2\|0\|5 | 7 | 2.33 | 2\|2\|0 | 0.15 | 0.44 | 0.15 | 0.44 | 0.15 | −7.85 | 1.75 | 0.63 |
| O95486 | SEC24A | 2\|3\|2 | 7 | 2.33 | 2\|0\|0 | 0.15 | 0.23 | 0.15 | 0.23 | 0.15 | −2.08 | 3.5 | 0.63 |
| O95630-2 | STAMBP | 3\|3\|0 | 6 | 2 | 0\|0\|2 | 0.15 | 0.23 | 0.15 | 0.23 | 0.15 | −5.43 | 3 | 0.63 |
| P57678 | GEMIN4 | 5\|2\|0 | 7 | 2.33 | 0\|0\|3 | 0.13 | 0.34 | 0.13 | 0.34 | 0.13 | −5.85 | 2.33 | 0.63 |
| Q9UNL2 | SSR3 | 3\|3\|3 | 9 | 3 | 3\|0\|0 | 0.13 | 0.13 | 0.13 | 0.13 | 0.13 | −1.89 | 3 | 0.63 |
| P49840 | GSK3A | 4\|0\|3 | 7 | 2.33 | 0\|0\|3 | 0.12 | 0.24 | 0.12 | 0.24 | 0.12 | −5.85 | 2.33 | 0.63 |
| O00399 | DCTN6 | 0\|2\|3 | 5 | 1.67 | 0\|0\|2 | 0.11 | 0.23 | 0.11 | 0.23 | 0.11 | −5.43 | 2.5 | 0.63 |
| Q9HB19 | PLEKHA2 | 2\|2\|4 | 8 | 2.67 | 0\|0\|3 | 0.11 | 0.24 | 0.11 | 0.24 | 0.11 | −2.93 | 2.67 | 0.64 |
| O14757-2 | CHEK1 | 2\|0\|3 | 5 | 1.67 | 0\|0\|2 | 0.11 | 0.23 | 0.11 | 0.23 | 0.11 | −5.43 | 2.5 | 0.63 |
| Q04446 | GBE1 | 3\|2\|0 | 5 | 1.67 | 0\|2\|0 | 0.11 | 0.23 | 0.11 | 0.23 | 0.11 | −5.43 | 2.5 | 0.63 |
| Q9BYD3-2 | MRPL4 | 2\|0\|3 | 5 | 1.67 | 2\|0\|0 | 0.11 | 0.23 | 0.11 | 0.23 | 0.11 | −5.43 | 2.5 | 0.63 |
| Q5T7N3 | KANK4 | 5\|5\|4 | 14 | 4.67 | 0\|2\|3 | 0.11 | 0.14 | 0.11 | 0.14 | 0.11 | −3.02 | 2.8 | 0.65 |
| Q9Y3A4 | RRP7A | 0\|3\|2 | 5 | 1.67 | 2\|0\|0 | 0.11 | 0.23 | 0.11 | 0.23 | 0.11 | −5.43 | 2.5 | 0.63 |
| A8CG34 | POM121C | 3\|2\|0 | 5 | 1.67 | 0\|0\|2 | 0.11 | 0.23 | 0.11 | 0.23 | 0.11 | −5.43 | 2.5 | 0.63 |
| Q6ZNB6 | NFXL1 | 4\|3\|4 | 11 | 3.67 | 2\|0\|2 | 0.11 | 0.15 | 0.11 | 0.15 | 0.11 | −3.25 | 2.75 | 0.65 |
| P38606-2 | ATP6V1A | 0\|2\|3 | 5 | 1.67 | 2\|0\|0 | 0.11 | 0.23 | 0.11 | 0.23 | 0.11 | −5.43 | 2.5 | 0.63 |
| Q5TCZ1-3 | SH3PXD2A | 2\|0\|3 | 5 | 1.67 | 0\|0\|2 | 0.11 | 0.23 | 0.11 | 0.23 | 0.11 | −5.43 | 2.5 | 0.63 |
| O15460 | P4HA2 | 2\|0\|3 | 5 | 1.67 | 0\|2\|0 | 0.11 | 0.23 | 0.11 | 0.23 | 0.11 | −5.43 | 2.5 | 0.63 |
| Q95604 | HLA-C | 4\|2\|0 | 6 | 2 | 0\|3\|0 | 0.1 | 0.24 | 0.1 | 0.24 | 0.1 | −5.85 | 2 | 0.65 |
| P14923 | JUP | 8\|2\|2 | 12 | 4 | 3\|4\|0 | 0.09 | 0.27 | 0.09 | 0.27 | 0.09 | −7.98 | 1.71 | 0.65 |
| Q8IZT6-2 | ASPM | 0\|0\|3 | 3 | 1 | 0\|0\|2 | 0.08 | 0.23 | 0.08 | 0.23 | 0.08 | −5.43 | 1.5 | 0.66 |
| P49247 | RPIA | 0\|0\|3 | 3 | 1 | 2\|0\|0 | 0.08 | 0.23 | 0.08 | 0.23 | 0.08 | −5.43 | 1.5 | 0.66 |
| P01116-2 | KRAS | 5\|0\|0 | 5 | 1.67 | 0\|0\|4 | 0.08 | 0.24 | 0.08 | 0.24 | 0.08 | −7.33 | 1.25 | 0.65 |
| Q9UKK3 | PARP4 | 0\|0\|3 | 3 | 1 | 0\|0\|2 | 0.08 | 0.23 | 0.08 | 0.23 | 0.08 | −5.43 | 1.5 | 0.66 |
| Q8NEC7-3 | GSTCD | 0\|0\|3 | 3 | 1 | 2\|0\|0 | 0.08 | 0.23 | 0.08 | 0.23 | 0.08 | −5.43 | 1.5 | 0.66 |
| Q12968 | NFATC3 | 0\|0\|3 | 3 | 1 | 0\|2\|0 | 0.08 | 0.23 | 0.08 | 0.23 | 0.08 | −5.43 | 1.5 | 0.66 |
| Q15813 | TBCE | 3\|2\|2 | 7 | 2.33 | 0\|3\|0 | 0.08 | 0.13 | 0.08 | 0.13 | 0.08 | −2.93 | 2.33 | 0.66 |
| P42694 | HELZ | 2\|3\|2 | 7 | 2.33 | 3\|0\|0 | 0.08 | 0.13 | 0.08 | 0.13 | 0.08 | −2.93 | 2.33 | 0.66 |
| P51692 | STAT5B | 0\|0\|3 | 3 | 1 | 0\|0\|2 | 0.08 | 0.23 | 0.08 | 0.23 | 0.08 | −5.43 | 1.5 | 0.66 |
| Q9BYJ9 | YTHDF1 | 0\|4\|0 | 4 | 1.33 | 3\|0\|0 | 0.08 | 0.24 | 0.08 | 0.24 | 0.08 | −5.85 | 1.33 | 0.65 |
| Q99575 | POP1 | 0\|0\|3 | 3 | 1 | 2\|0\|0 | 0.08 | 0.23 | 0.08 | 0.23 | 0.08 | −5.43 | 1.5 | 0.66 |
| Q15650 | TRIP4 | 3\|0\|0 | 3 | 1 | 2\|0\|0 | 0.08 | 0.23 | 0.08 | 0.23 | 0.08 | −5.43 | 1.5 | 0.66 |
| P36871 | PGM1 | 8\|5\|4 | 17 | 5.67 | 4\|2\|2 | 0.08 | 0.23 | 0.08 | 0.23 | 0.08 | −6.42 | 2.13 | 0.65 |
| Q15477 | SKIV2L | 3\|0\|0 | 3 | 1 | 2\|0\|0 | 0.08 | 0.23 | 0.08 | 0.23 | 0.08 | −5.43 | 1.5 | 0.66 |
| Q61A86-2 | ELP | 0\|3\|0 | 3 | 1 | 0\|0\|2 | 0.08 | 0.23 | 0.08 | 0.23 | 0.08 | −5.43 | 1.5 | 0.66 |
| O15084 | ANKRD28 | 0\|3\|0 | 3 | 1 | 2\|0\|0 | 0.08 | 0.23 | 0.08 | 0.23 | 0.08 | −5.43 | 1.5 | 0.66 |
| P51812 | RPS6KA3 | 0\|4\|0 | 4 | 1.33 | 0\|0\|3 | 0.08 | 0.24 | 0.08 | 0.24 | 0.08 | −5.85 | 1.33 | 0.65 |
| O95861-4 | BPNT1 | 0\|0\|3 | 3 | 1 | 2\|0\|0 | 0.08 | 0.23 | 0.08 | 0.23 | 0.08 | −5.43 | 1.5 | 0.66 |
| Q9H583 | HEATR1 | 0\|3\|0 | 3 | 1 | 0\|0\|2 | 0.08 | 0.23 | 0.08 | 0.23 | 0.08 | −5.43 | 1.5 | 0.66 |
| 014744-4 | PRMT5 | 0\|0\|3 | 3 | 1 | 2\|0\|0 | 0.08 | 0.23 | 0.08 | 0.23 | 0.08 | −5.43 | 1.5 | 0.66 |
| Q9UIV1-2 | CNOT7 | 0\|3\|0 | 3 | 1 | 0\|2\|0 | 0.08 | 0.23 | 0.08 | 0.23 | 0.08 | −5.43 | 1.5 | 0.66 |
| Q5D862 | FLG2 | 0\|0\|5 | 5 | 1.67 | 0\|4\|0 | 0.08 | 0.24 | 0.08 | 0.24 | 0.08 | −7.33 | 1.25 | 0.65 |
| P99999 | CYCS | 0\|2\|2 | 4 | 1.33 | 0\|2\|0 | 0.07 | 0.11 | 0.07 | 0.11 | 0.07 | −5.43 | 2 | 0.68 |
| O60942 | RNGTT | 0\|2\|2 | 4 | 1.33 | 0\|0\|2 | 0.07 | 0.11 | 0.07 | 0.11 | 0.07 | −5.43 | 2 | 0.68 |
| O15357-2 | INPPL1 | 0\|2\|2 | 4 | 1.33 | 2\|0\|0 | 0.07 | 0.11 | 0.07 | 0.11 | 0.07 | −5.43 | 2 | 0.68 |
| Q16698-2 | DECR1 | 0\|2\|2 | 4 | 1.33 | 0\|0\|2 | 0.07 | 0.11 | 0.07 | 0.11 | 0.07 | −5.43 | 2 | 0.68 |
| Q9HCD6 | TANC2 | 2\|0\|2 | 4 | 1.33 | 2\|0\|0 | 0.07 | 0.11 | 0.07 | 0.11 | 0.07 | −5.43 | 2 | 0.68 |
| P10606 | COX5B | 2\|2\|0 | 4 | 1.33 | 0\|0\|2 | 0.07 | 0.11 | 0.07 | 0.11 | 0.07 | −5.43 | 2 | 0.68 |
| P78406 | RAE1 | 4\|2\|3 | 9 | 3 | 4\|0\|0 | 0.07 | 0.14 | 0.07 | 0.14 | 0.07 | −3.92 | 2.25 | 0.68 |
| P04083 | ANXA1 | 6\|5\|4 | 15 | 5 | 4\|2\|0 | 0.07 | 0.13 | 0.07 | 0.13 | 0.07 | −3.99 | 2.5 | 0.69 |
| Q86UU1-3 | PHLDB1 | 2\|0\|2 | 4 | 1.33 | 2\|0\|0 | 0.07 | 0.11 | 0.07 | 0.11 | 0.07 | −5.43 | 2 | 0.68 |
| Q9H6T3-2 | RPAP3 | 2\|0\|2 | 4 | 1.33 | 2\|0\|0 | 0.07 | 0.11 | 0.07 | 0.11 | 0.07 | −5.43 | 2 | 0.68 |
| P02452 | COL1A1 | 0\|2\|2 | 4 | 1.33 | 0\|2\|0 | 0.07 | 0.11 | 0.07 | 0.11 | 0.07 | −5.43 | 2 | 0.68 |
| Q9UK76 | JPT1 | 2\|0\|2 | 4 | 1.33 | 2\|0\|0 | 0.07 | 0.11 | 0.07 | 0.11 | 0.07 | −5.43 | 2 | 0.68 |
| Q8NFF5-3 | FLAD1 | 2\|0\|2 | 4 | 1.33 | 0\|2\|0 | 0.07 | 0.11 | 0.07 | 0.11 | 0.07 | −5.43 | 2 | 0.68 |
| P55735-2 | SEC13 | 2\|2\|0 | 4 | 1.33 | 0\|0\|2 | 0.07 | 0.11 | 0.07 | 0.11 | 0.07 | −5.43 | 2 | 0.68 |
| Q9H0S4 | DDX47 | 2\|3\|0 | 5 | 1.67 | 0\|3\|0 | 0.06 | 0.13 | 0.06 | 0.13 | 0.06 | −5.85 | 1.67 | 0.69 |
| O15460-2 | P4HA2 | 2\|0\|3 | 5 | 1.67 | 0\|3\|0 | 0.06 | 0.13 | 0.06 | 0.13 | 0.06 | −5.85 | 1.67 | 0.69 |
| Q53GA4 | PHLDA2 | 4\|2\|2 | 8 | 2.67 | 2\|2\|0 | 0.06 | 0.15 | 0.06 | 0.15 | 0.06 | −4.72 | 2 | 0.7 |
| Q8NC60 | NOA1 | 3\|2\|4 | 9 | 3 | 2\|2\|0 | 0.06 | 0.15 | 0.06 | 0.15 | 0.06 | −4.72 | 2.25 | 0.69 |
| Q15031 | LARS2 | 3\|3\|3 | 9 | 3 | 4\|0\|0 | 0.06 | 0.06 | 0.06 | 0.06 | 0.06 | −2.7 | 2.25 | 0.69 |
| Q6DKJ4-3 | NXN | 2\|4\|2 | 8 | 2.67 | 0\|2\|2 | 0.06 | 0.15 | 0.06 | 0.15 | 0.06 | −4.72 | 2 | 0.7 |
| P13798 | APEH | 0\|2\|3 | 5 | 1.67 | 3\|0\|0 | 0.06 | 0.13 | 0.06 | 0.13 | 0.06 | −5.85 | 1.67 | 0.69 |
| Q14558 | PRPSAP1 | 2\|4\|3 | 9 | 3 | 2\|0\|2 | 0.06 | 0.15 | 0.06 | 0.15 | 0.06 | −4.72 | 2.25 | 0.69 |
| P50750 | CDK9 | 3\|2\|0 | 5 | 1.67 | 0\|3\|0 | 0.06 | 0.13 | 0.06 | 0.13 | 0.06 | −5.85 | 1.67 | 0.69 |
| Q5SRE5 | NUP188 | 2\|5\|2 | 9 | 3 | 0\|2\|3 | 0.05 | 0.14 | 0.05 | 0.14 | 0.05 | −5.71 | 1.8 | 0.71 |
| O14976-2 | GAK | 0\|3\|5 | 8 | 2.67 | 3\|0\|2 | 0.05 | 0.14 | 0.05 | 0.14 | 0.05 | −9.18 | 1.6 | 0.7 |
| P29372-5 | MPG | 3\|6\|4 | 13 | 4.33 | 4\|2\|0 | 0.05 | 0.13 | 0.05 | 0.13 | 0.05 | −5.25 | 2.17 | 0.7 |
| Q8N543 | OGFOD1 | 4\|0\|2 | 6 | 2 | 2\|0\|2 | 0.05 | 0.15 | 0.05 | 0.15 | 0.05 | −7.85 | 1.5 | 0.7 |
| P14735 | IDE | 0\|2\|4 | 6 | 2 | 2\|0\|2 | 0.05 | 0.15 | 0.05 | 0.15 | 0.05 | −7.85 | 1.5 | 0.7 |
| Q9HAH7 | FBRS | 2\|2\|2 | 6 | 2 | 0\|3\|0 | 0.05 | 0.05 | 0.05 | 0.05 | 0.05 | −2.93 | 2 | 0.7 |
| P45974-2 | USP5 | 6\|4\|4 | 14 | 4.67 | 4\|2\|0 | 0.05 | 0.13 | 0.05 | 0.13 | 0.05 | −3.99 | 2.33 | 0.7 |
| Q92598-2 | HSPH1 | 2\|2\|5 | 9 | 3 | 0\|5\|0 | 0.05 | 0.14 | 0.05 | 0.14 | 0.05 | −5.01 | 1.8 | 0.7 |
| Q14974 | KPNB1 | 0\|2\|4 | 6 | 2 | 2\|2\|0 | 0.05 | 0.15 | 0.05 | 0.15 | 0.05 | −7.85 | 1.5 | 0.7 |
| Q7Z2E3-7 | APTX | 0\|0\|3 | 3 | 1 | 3\|0\|0 | 0.04 | 0.13 | 0.04 | 0.13 | 0.04 | −5.85 | 1 | 0.71 |
| P61421 | ATP6V0D1 | 0\|2\|0 | 2 | 0.67 | 0\|2\|0 | 0.04 | 0.11 | 0.04 | 0.11 | 0.04 | −5.43 | 1 | 0.71 |
| Q9P0U3-2 | SENP1 | 2\|0\|0 | 2 | 0.67 | 2\|0\|0 | 0.04 | 0.11 | 0.04 | 0.11 | 0.04 | −5.43 | 1 | 0.71 |
| Q04726-2 | TLE3 | 0\|2\|0 | 2 | 0.67 | 0\|2\|0 | 0.04 | 0.11 | 0.04 | 0.11 | 0.04 | −5.43 | 1 | 0.71 |
| Q9Y311 | FBXO7 | 0\|0\|2 | 2 | 0.67 | 2\|0\|0 | 0.04 | 0.11 | 0.04 | 0.11 | 0.04 | −5.43 | 1 | 0.71 |
| Q8TCJ2 | STT3B | 2\|0\|0 | 2 | 0.67 | 0\|0\|2 | 0.04 | 0.11 | 0.04 | 0.11 | 0.04 | −5.43 | 1 | 0.71 |
| P62633-2 | CNBP | 0\|0\|3 | 3 | 1 | 3\|0\|0 | 0.04 | 0.13 | 0.04 | 0.13 | 0.04 | −5.85 | 1 | 0.71 |
| P13674-3 | P4HA1 | 3\|7\|6 | 16 | 5.33 | 2\|3\|3 | 0.04 | 0.08 | 0.04 | 0.08 | 0.04 | −7.86 | 2 | 0.75 |
| Q9NPI6-2 | DCP1A | 3\|0\|0 | 3 | 1 | 3\|0\|0 | 0.04 | 0.13 | 0.04 | 0.13 | 0.04 | −5.85 | 1 | 0.71 |
| Q12769 | NUP160 | 0\|2\|0 | 2 | 0.67 | 0\|2\|0 | 0.04 | 0.11 | 0.04 | 0.11 | 0.04 | −5.43 | 1 | 0.71 |
| P25325 | MPST | 2\|0\|0 | 2 | 0.67 | 0\|0\|2 | 0.04 | 0.11 | 0.04 | 0.11 | 0.04 | −5.43 | 1 | 0.71 |
| O75319 | DUSP11 | 0\|2\|0 | 2 | 0.67 | 0\|2\|0 | 0.04 | 0.11 | 0.04 | 0.11 | 0.04 | −5.43 | 1 | 0.71 |
| Q13625-2 | TP53BP2 | 2\|0\|0 | 2 | 0.67 | 2\|0\|0 | 0.04 | 0.11 | 0.04 | 0.11 | 0.04 | −5.43 | 1 | 0.71 |
| P05091-2 | ALDH2 | 0\|0\|2 | 2 | 0.67 | 0\|2\|0 | 0.04 | 0.11 | 0.04 | 0.11 | 0.04 | −5.43 | 1 | 0.71 |
| P61163 | ACTR1A | 2\|0\|0 | 2 | 0.67 | 0\|0\|2 | 0.04 | 0.11 | 0.04 | 0.11 | 0.04 | −5.43 | 1 | 0.71 |
| Q9GZT9-2 | EGLN1 | 0\|2\|0 | 2 | 0.67 | 0\|0\|2 | 0.04 | 0.11 | 0.04 | 0.11 | 0.04 | −5.43 | 1 | 0.71 |
| O60573-2 | EIF4E2 | 0\|0\|2 | 2 | 0.67 | 2\|0\|0 | 0.04 | 0.11 | 0.04 | 0.11 | 0.04 | −5.43 | 1 | 0.71 |
| Q14669-4 | TRIP12 | 2\|0\|0 | 2 | 0.67 | 2\|0\|0 | 0.04 | 0.11 | 0.04 | 0.11 | 0.04 | −5.43 | 1 | 0.71 |
| Q96125 | RBM17 | 0\|2\|0 | 2 | 0.67 | 2\|0\|0 | 0.04 | 0.11 | 0.04 | 0.11 | 0.04 | −5.43 | 1 | 0.71 |
| O95373 | IPO7 | 2\|0\|0 | 2 | 0.67 | 0\|0\|2 | 0.04 | 0.11 | 0.04 | 0.11 | 0.04 | −5.43 | 1 | 0.71 |
| O00541-2 | PES1 | 0\|2\|0 | 2 | 0.67 | 0\|2\|0 | 0.04 | 0.11 | 0.04 | 0.11 | 0.04 | −5.43 | 1 | 0.71 |
| P78318 | IGBP1 | 0\|0\|2 | 2 | 0.67 | 0\|0\|2 | 0.04 | 0.11 | 0.04 | 0.11 | 0.04 | −5.43 | 1 | 0.71 |
| Q5JTV8 | TOR1AIP1 | 0\|0\|2 | 2 | 0.67 | 2\|0\|0 | 0.04 | 0.11 | 0.04 | 0.11 | 0.04 | −5.43 | 1 | 0.71 |
| Q9H4A4 | RNPEP | 0\|2\|0 | 2 | 0.67 | 0\|0\|2 | 0.04 | 0.11 | 0.04 | 0.11 | 0.04 | −5.43 | 1 | 0.71 |
| P25490 | YY1 | 2\|0\|0 | 2 | 0.67 | 0\|2\|0 | 0.04 | 0.11 | 0.04 | 0.11 | 0.04 | −5.43 | 1 | 0.71 |
| P48163-2 | ME1 | 0\|0\|2 | 2 | 0.67 | 0\|2\|0 | 0.04 | 0.11 | 0.04 | 0.11 | 0.04 | −5.43 | 1 | 0.71 |
| Q9NUL7 | DDX28 | 0\|0\|2 | 2 | 0.67 | 2\|0\|0 | 0.04 | 0.11 | 0.04 | 0.11 | 0.04 | −5.43 | 1 | 0.71 |
| Q02413 | DSG1 | 2\|0\|0 | 2 | 0.67 | 0\|2\|0 | 0.04 | 0.11 | 0.04 | 0.11 | 0.04 | −5.43 | 1 | 0.71 |
| Q15555-4 | MAPRE2 | 0\|0\|2 | 2 | 0.67 | 0\|0\|2 | 0.04 | 0.11 | 0.04 | 0.11 | 0.04 | −5.43 | 1 | 0.71 |
| Q8NBT2-2 | SPC24 | 2\|0\|0 | 2 | 0.67 | 0\|0\|2 | 0.04 | 0.11 | 0.04 | 0.11 | 0.04 | −5.43 | 1 | 0.71 |
| Q96KP1 | EXOC2 | 0\|2\|0 | 2 | 0.67 | 0\|2\|0 | 0.04 | 0.11 | 0.04 | 0.11 | 0.04 | −5.43 | 1 | 0.71 |
| P08134 | RHOC | 0\|0\|2 | 2 | 0.67 | 0\|0\|2 | 0.04 | 0.11 | 0.04 | 0.11 | 0.04 | −5.43 | 1 | 0.71 |
| P33981-2 | TTK | 6\|2\|6 | 14 | 4.67 | 2\|0\|5 | 0.04 | 0.06 | 0.04 | 0.06 | 0.04 | −7.81 | 2 | 0.71 |
| Q9BZV1-2 | UBXN6 | 0\|2\|0 | 2 | 0.67 | 0\|0\|2 | 0.04 | 0.11 | 0.04 | 0.11 | 0.04 | −5.43 | 1 | 0.71 |
| O75530-3 | EED | 0\|2\|0 | 2 | 0.67 | 2\|0\|0 | 0.04 | 0.11 | 0.04 | 0.11 | 0.04 | −5.43 | 1 | 0.71 |
| Q8N1N4 | KRT78 | 2\|0\|0 | 2 | 0.67 | 2\|0\|0 | 0.04 | 0.11 | 0.04 | 0.11 | 0.04 | −5.43 | 1 | 0.71 |
| Q8NEZ5 | FBXO22 | 2\|0\|0 | 2 | 0.67 | 2\|0\|0 | 0.04 | 0.11 | 0.04 | 0.11 | 0.04 | −5.43 | 1 | 0.71 |
| Q9NWT8 | AURKAIP1 | 0\|0\|3 | 3 | 1 | 3\|0\|0 | 0.04 | 0.13 | 0.04 | 0.13 | 0.04 | −5.85 | 1 | 0.71 |
| Q8NBM4 | UBAC2 | 0\|2\|0 | 2 | 0.67 | 2\|0\|0 | 0.04 | 0.11 | 0.04 | 0.11 | 0.04 | −5.43 | 1 | 0.71 |
| Q86WB0 | ZC3HC1 | 2\|0\|0 | 2 | 0.67 | 0\|0\|2 | 0.04 | 0.11 | 0.04 | 0.11 | 0.04 | −5.43 | 1 | 0.71 |
| Q9Y673-2 | ALG5 | 0\|0\|2 | 2 | 0.67 | 0\|0\|2 | 0.04 | 0.11 | 0.04 | 0.11 | 0.04 | −5.43 | 1 | 0.71 |
| Q9GZZ9 | UBA5 | 0\|2\|0 | 2 | 0.67 | 0\|2\|0 | 0.04 | 0.11 | 0.04 | 0.11 | 0.04 | −5.43 | 1 | 0.71 |
| Q9C0C7-4 | AMBRA1 | 0\|2\|0 | 2 | 0.67 | 2\|0\|0 | 0.04 | 0.11 | 0.04 | 0.11 | 0.04 | −5.43 | 1 | 0.71 |
| O00159-2 | MYO1C | 2\|0\|0 | 2 | 0.67 | 2\|0\|0 | 0.04 | 0.11 | 0.04 | 0.11 | 0.04 | −5.43 | 1 | 0.71 |
| Q86Y07-4 | VRK2 | 0\|0\|2 | 2 | 0.67 | 0\|2\|0 | 0.04 | 0.11 | 0.04 | 0.11 | 0.04 | −5.43 | 1 | 0.71 |
| P35611-2 | ADD1 | 0\|0\|2 | 2 | 0.67 | 2\|0\|0 | 0.04 | 0.11 | 0.04 | 0.11 | 0.04 | −5.43 | 1 | 0.71 |
| P06730 | EIF4E | 0\|0\|2 | 2 | 0.67 | 0\|2\|0 | 0.04 | 0.11 | 0.04 | 0.11 | 0.04 | −5.43 | 1 | 0.71 |
| Q96NW4 | ANKRD27 | 0\|0\|2 | 2 | 0.67 | 2\|0\|0 | 0.04 | 0.11 | 0.04 | 0.11 | 0.04 | −5.43 | 1 | 0.71 |
| Q7L1Q6-2 | BZW1 | 2\|0\|0 | 2 | 0.67 | 0\|2\|0 | 0.04 | 0.11 | 0.04 | 0.11 | 0.04 | −5.43 | 1 | 0.71 |
| P00492 | HPRT1 | 0\|0\|2 | 2 | 0.67 | 2\|0\|0 | 0.04 | 0.11 | 0.04 | 0.11 | 0.04 | −5.43 | 1 | 0.71 |
| P49590 | HARS2 | 0\|0\|2 | 2 | 0.67 | 2\|0\|0 | 0.04 | 0.11 | 0.04 | 0.11 | 0.04 | −5.43 | 1 | 0.71 |
| P27105 | STOM | 2\|0\|0 | 2 | 0.67 | 0\|0\|2 | 0.04 | 0.11 | 0.04 | 0.11 | 0.04 | −5.43 | 1 | 0.71 |
| O95677-5 | EYA4 | 2\|0\|0 | 2 | 0.67 | 0\|2\|0 | 0.04 | 0.11 | 0.04 | 0.11 | 0.04 | −5.43 | 1 | 0.71 |
| Q8N5F7 | NKAP | 2\|3\|2 | 7 | 2.33 | 0\|4\|0 | 0.03 | 0.06 | 0.03 | 0.06 | 0.03 | −3.92 | 1.75 | 0.75 |
| O43252 | PAPSS1 | 5\|4\|6 | 15 | 5 | 2\|2\|3 | 0.03 | 0.07 | 0.03 | 0.07 | 0.03 | −5.27 | 2.14 | 0.75 |
| Q9BZE1 | MRPL37 | 0\|2\|3 | 5 | 1.67 | 4\|0\|0 | 0.03 | 0.06 | 0.03 | 0.06 | 0.03 | −7.33 | 1.25 | 0.76 |
| O95376 | ARIH2 | 2\|2\|0 | 4 | 1.33 | 3\|0\|0 | 0.03 | 0.05 | 0.03 | 0.05 | 0.03 | −5.85 | 1.33 | 0.75 |
| P52298-3 | NCBP2 | 0\|4\|4 | 8 | 2.67 | 2\|0\|3 | 0.03 | 0.05 | 0.03 | 0.05 | 0.03 | −9.18 | 1.6 | 0.75 |
| Q9Y262 | EIF3L | 0\|2\|2 | 4 | 1.33 | 0\|3\|0 | 0.03 | 0.05 | 0.03 | 0.05 | 0.03 | −5.85 | 1.33 | 0.75 |
| O95817 | BAG3 | 4\|5\|8 | 17 | 5.67 | 3\|4\|2 | 0.03 | 0.1 | 0.03 | 0.1 | 0.03 | −7.59 | 1.89 | 0.75 |
| Q96Q11-2 | TRNT1 | 2\|3\|3 | 8 | 2.67 | 2\|0\|2 | 0.03 | 0.04 | 0.03 | 0.04 | 0.03 | −4.72 | 2 | 0.76 |
| P13674 | P4HA1 | 4\|7\|5 | 16 | 5.33 | 2\|3\|3 | 0.03 | 0.08 | 0.03 | 0.08 | 0.03 | −6.42 | 2 | 0.75 |
| Q96SK2-3 | TMEM209 | 0\|2\|2 | 4 | 1.33 | 0\|0\|3 | 0.03 | 0.05 | 0.03 | 0.05 | 0.03 | −5.85 | 1.33 | 0.75 |
| P41240 | CSK | 5\|4\|4 | 13 | 4.33 | 3\|3\|0 | 0.03 | 0.05 | 0.03 | 0.05 | 0.03 | −4.09 | 2.17 | 0.76 |
| Q9H9Y6-5 | POLR1B | 2\|3\|2 | 7 | 2.33 | 4\|0\|0 | 0.03 | 0.06 | 0.03 | 0.06 | 0.03 | −3.92 | 1.75 | 0.75 |
| Q99816-2 | TSG101 | 2\|2\|0 | 4 | 1.33 | 3\|0\|0 | 0.03 | 0.05 | 0.03 | 0.05 | 0.03 | −5.85 | 1.33 | 0.75 |
| Q96HA1 | POM121 | 2\|0\|0 | 2 | 0.67 | 0\|0\|3 | 0.02 | 0.05 | 0.02 | 0.05 | 0.02 | −5.85 | 0.67 | 0.77 |
| Q9UGP4 | LIMD1 | 3\|2\|2 | 7 | 2.33 | 0\|2\|2 | 0.02 | 0.04 | 0.02 | 0.04 | 0.02 | −4.72 | 1.75 | 0.77 |
| P61619 | SEC61A1 | 4\|3\|2 | 9 | 3 | 0\|2\|3 | 0.02 | 0.05 | 0.02 | 0.05 | 0.02 | −5.71 | 1.8 | 0.76 |
| Q01968 | OCRL | 3\|2\|0 | 5 | 1.67 | 0\|2\|2 | 0.02 | 0.04 | 0.02 | 0.04 | 0.02 | −7.85 | 1.25 | 0.78 |
| Q9H9T3-2 | ELP3 | 0\|0\|2 | 2 | 0.67 | 0\|0\|3 | 0.02 | 0.05 | 0.02 | 0.05 | 0.02 | −5.85 | 0.67 | 0.77 |
| Q86TB9-4 | PATL1 | 2\|0\|0 | 2 | 0.67 | 0\|0\|3 | 0.02 | 0.05 | 0.02 | 0.05 | 0.02 | −5.85 | 0.67 | 0.77 |
| Q9NVV4 | MTPAP | 2\|0\|3 | 5 | 1.67 | 0\|2\|2 | 0.02 | 0.04 | 0.02 | 0.04 | 0.02 | −7.85 | 1.25 | 0.78 |
| P48059 | LIMS1 | 0\|2\|0 | 2 | 0.67 | 0\|0\|3 | 0.02 | 0.05 | 0.02 | 0.05 | 0.02 | −5.85 | 0.67 | 0.77 |
| Q9Y653-5 | ADGRG1 | 2\|3\|0 | 5 | 1.67 | 0\|2\|2 | 0.02 | 0.04 | 0.02 | 0.04 | 0.02 | −7.85 | 1.25 | 0.78 |
| Q16513-3 | PKN2 | 0\|0\|4 | 4 | 1.33 | 2\|3\|0 | 0.02 | 0.05 | 0.02 | 0.05 | 0.02 | −9.18 | 0.8 | 0.78 |
| Q96IJ6 | GMPPA | 3\|0\|2 | 5 | 1.67 | 0\|2\|2 | 0.02 | 0.04 | 0.02 | 0.04 | 0.02 | −7.85 | 1.25 | 0.78 |
| Q9UKV8 | AGO2 | 4\|2\|2 | 8 | 2.67 | 3\|0\|2 | 0.02 | 0.05 | 0.02 | 0.05 | 0.02 | −5.71 | 1.6 | 0.77 |
| O43592 | XPOT | 2\|0\|3 | 5 | 1.67 | 0\|2\|2 | 0.02 | 0.04 | 0.02 | 0.04 | 0.02 | −7.85 | 1.25 | 0.78 |
| Q13418 | ILK | 3\|2\|0 | 5 | 1.67 | 2\|2\|0 | 0.02 | 0.04 | 0.02 | 0.04 | 0.02 | −7.85 | 1.25 | 0.78 |
| P55036 | PSMD4 | 4\|2\|5 | 11 | 3.67 | 0\|2\|4 | 0.02 | 0.05 | 0.02 | 0.05 | 0.02 | −6.72 | 1.83 | 0.76 |
| P61224-3 | RAP1B | 4\|2\|2 | 8 | 2.67 | 0\|2\|3 | 0.02 | 0.05 | 0.02 | 0.05 | 0.02 | −5.71 | 1.6 | 0.77 |
| Q13951 | CBFB | 2\|3\|2 | 7 | 2.33 | 2\|0\|2 | 0.02 | 0.04 | 0.02 | 0.04 | 0.02 | −4.72 | 1.75 | 0.77 |
| Q9Y3B2 | EXOSC1 | 2\|4\|3 | 9 | 3 | 3\|2\|0 | 0.02 | 0.05 | 0.02 | 0.05 | 0.02 | −5.71 | 1.8 | 0.76 |
| Q8WUM4 | PDCD6IP | 0\|4\|0 | 4 | 1.33 | 2\|0\|3 | 0.02 | 0.05 | 0.02 | 0.05 | 0.02 | −9.18 | 0.8 | 0.78 |
| Q9NQH7-2 | XPNPEP3 | 2\|0\|0 | 2 | 0.67 | 0\|0\|3 | 0.02 | 0.05 | 0.02 | 0.05 | 0.02 | −5.85 | 0.67 | 0.77 |
| O14818 | PSMA7 | 3\|3\|0 | 6 | 2 | 0\|2\|2 | 0.02 | 0.04 | 0.02 | 0.04 | 0.02 | −7.85 | 1.5 | 0.76 |
| Q13951-2 | CBFB | 3\|2\|2 | 7 | 2.33 | 2\|0\|2 | 0.02 | 0.04 | 0.02 | 0.04 | 0.02 | −4.72 | 1.75 | 0.77 |
| P51553 | IDH3G | 2\|2\|3 | 7 | 2.33 | 2\|2\|0 | 0.02 | 0.04 | 0.02 | 0.04 | 0.02 | 4.72 | 1.75 | 0.77 |
| Q14195 | DPYSL3 | 2\|0\|0 | 2 | 0.67 | 0\|3\|0 | 0.02 | 0.05 | 0.02 | 0.05 | 0.02 | −5.85 | 0.67 | 0.77 |
| Q9H910 | JPT2 | 5\|3\|3 | 11 | 3.67 | 0\|3\|3 | 0.02 | 0.05 | 0.02 | 0.05 | 0.02 | −5.41 | 1.83 | 0.76 |
| Q14847 | LASP1 | 3\|2\|6 | 11 | 3.67 | 2\|2\|3 | 0.02 | 0.07 | 0.02 | 0.07 | 0.02 | −8.15 | 1.57 | 0.76 |
| O60841 | EIF5B | 2\|0\|4 | 6 | 2 | 2\|3\|0 | 0.02 | 0.05 | 0.02 | 0.05 | 0.02 | −9.18 | 1.2 | 0.78 |
| P17706-2 | PTPN2 | 4\|5\|2 | 11 | 3.67 | 3\|0\|3 | 0.02 | 0.05 | 0.02 | 0.05 | 0.02 | −6.87 | 1.83 | 0.76 |
| Q86X95-2 | CIR1 | 3\|0\|3 | 6 | 2 | 0\|2\|2 | 0.02 | 0.04 | 0.02 | 0.04 | 0.02 | −7.85 | 1.5 | 0.76 |
| Q15369-2 | ELOC | 0\|2\|3 | 5 | 1.67 | 2\|2\|0 | 0.02 | 0.04 | 0.02 | 0.04 | 0.02 | −7.85 | 1.25 | 0.78 |
| Q8N0X7 | SPG20 | 2\|0\|0 | 2 | 0.67 | 0\|0\|3 | 0.02 | 0.05 | 0.02 | 0.05 | 0.02 | −5.85 | 0.67 | 0.77 |
| Q14643-4 | ITPR1 | 2\|0\|0 | 2 | 0.67 | 3\|0\|0 | 0.02 | 0.05 | 0.02 | 0.05 | 0.02 | −5.85 | 0.67 | 0.77 |
| Q8ND04 | SMG8 | 2\|0\|0 | 2 | 0.67 | 0\|0\|3 | 0.02 | 0.05 | 0.02 | 0.05 | 0.02 | −5.85 | 0.67 | 0.77 |
| P30086 | PEBP1 | 3\|0\|4 | 7 | 2.33 | 0\|2\|3 | 0.02 | 0.05 | 0.02 | 0.05 | 0.02 | −9.18 | 1.4 | 0.76 |
| Q6IQ22 | RAB12 | 0\|0\|3 | 3 | 1 | 4\|0\|0 | 0.02 | 0.06 | 0.02 | 0.06 | 0.02 | −7.33 | 0.75 | 0.76 |
| Q04206-2 | RELA | 2\|3\|4 | 9 | 3 | 3\|2\|0 | 0.02 | 0.05 | 0.02 | 0.05 | 0.02 | −5.71 | 1.8 | 0.76 |
| P17098 | ZNF8 | 3\|0\|2 | 5 | 1.67 | 2\|0\|2 | 0.02 | 0.04 | 0.02 | 0.04 | 0.02 | −7.85 | 1.25 | 0.78 |
| Q96GM8 | TOE1 | 0\|2\|0 | 2 | 0.67 | 0\|0\|3 | 0.02 | 0.05 | 0.02 | 0.05 | 0.02 | −5.85 | 0.67 | 0.77 |
| Q9Y570 | PPME1 | 6\|3\|2 | 11 | 3.67 | 3\|0\|4 | 0.02 | 0.06 | 0.02 | 0.06 | 0.02 | −7.98 | 1.57 | 0.76 |
| P61923 | COPZ1 | 3\|2\|0 | 5 | 1.67 | 0\|2\|2 | 0.02 | 0.04 | 0.02 | 0.04 | 0.02 | −7.85 | 1.25 | 0.78 |
| P61011-2 | SRP54 | 3\|0\|2 | 5 | 1.67 | 2\|2\|0 | 0.02 | 0.04 | 0.02 | 0.04 | 0.02 | −7.85 | 1.25 | 0.78 |
| Q8WVV9-5 | HNRNPLL | 6\|0\|6 | 12 | 4 | 3\|2\|3 | 0.02 | 0.02 | 0.02 | 0.02 | 0.02 | −13.44 | 1.5 | 0.78 |
| A1L0T0 | ILVBL | 3\|0\|3 | 6 | 2 | 2\|0\|2 | 0.02 | 0.04 | 0.02 | 0.04 | 0.02 | −7.85 | 1.5 | 0.76 |
| O75436 | VPS26A | 2\|0\|3 | 5 | 1.67 | 3\|0\|2 | 0.01 | 0.01 | 0.01 | 0.01 | 0.01 | −9.18 | 1 | 0.8 |
| Q96CS3 | FAF2 | 3\|0\|6 | 9 | 3 | 3\|2\|3 | 0.01 | 0.02 | 0.01 | 0.02 | 0.01 | −13.44 | 1.12 | 0.79 |
| O60762 | DPM1 | 2\|2\|0 | 4 | 1.33 | 2\|2\|0 | 0.01 | 0.01 | 0.01 | 0.01 | 0.01 | −7.85 | 1 | 0.8 |
| Q13347 | EIF31 | 3\|5\|0 | 8 | 2.67 | 3\|0\|4 | 0.01 | 0.02 | 0.01 | 0.02 | 0.01 | −11.81 | 1.14 | 0.79 |
| O75400-2 | PRPF40A | 4\|0\|3 | 7 | 2.33 | 0\|4\|2 | 0.01 | 0.02 | 0.01 | 0.02 | 0.01 | −10.24 | 1.17 | 0.79 |
| P10398 | ARAF | 2\|3\|0 | 5 | 1.67 | 3\|0\|2 | 0.01 | 0.01 | 0.01 | 0.01 | 0.01 | −9.18 | 1 | 0.8 |
| P50579-2 | METAP2 | 2\|0\|2 | 4 | 1.33 | 2\|0\|2 | 0.01 | 0.01 | 0.01 | 0.01 | 0.01 | −7.85 | 1 | 0.8 |
| Q9BV20 | MRI1 | 2\|3\|4 | 9 | 3 | 3\|0\|3 | 0.01 | 0.02 | 0.01 | 0.02 | 0.01 | −6.87 | 1.5 | 0.79 |
| P27695 | APEX1 | 2\|0\|4 | 6 | 2 | 3\|0\|3 | 0.01 | 0.02 | 0.01 | 0.02 | 0.01 | −10.39 | 1 | 0.81 |
| Q13330-2 | MTA1 | 0\|2\|3 | 5 | 1.67 | 2\|0\|3 | 0.01 | 0.01 | 0.01 | 0.01 | 0.01 | −9.18 | 1 | 0.8 |
| Q9H3P7 | ACBD3 | 3\|4\|2 | 9 | 3 | 2\|4\|0 | 0.01 | 0.02 | 0.01 | 0.02 | 0.01 | −6.72 | 1.5 | 0.79 |
| Q8IVF2-3 | AHNAK2 | 8\|5\|10 | 23 | 7.67 | 6\|4\|3 | 0.01 | 0.02 | 0.01 | 0.02 | 0.01 | −10.83 | 1.77 | 0.8 |
| Q9H078-4 | CLPB | 3\|0\|0 | 3 | 1 | 2\|0\|2 | 0.01 | 0.04 | 0.01 | 0.04 | 0.01 | −7.85 | 0.75 | 0.78 |
| Q14019 | COTL1 | 6\|2\|5 | 13 | 4.33 | 2\|2\|4 | 0.01 | 0.02 | 0.01 | 0.02 | 0.01 | −9.34 | 1.62 | 0.79 |
| Q7L5Y9-3 | MAEA | 0\|3\|2 | 5 | 1.67 | 2\|0\|3 | 0.01 | 0.01 | 0.01 | 0.01 | 0.01 | −9.18 | 1 | 0.8 |
| P67809 | YBX1 | 2\|7\|7 | 16 | 5.33 | 4\|2\|4 | 0.01 | 0.01 | 0.01 | 0.01 | 0.01 | −11.76 | 1.6 | 0.8 |
| Q9Y223-4 | GNE | 0\|2\|2 | 4 | 1.33 | 2\|0\|2 | 0.01 | 0.01 | 0.01 | 0.01 | 0.01 | −7.85 | 1 | 0.8 |
| P25787 | PSMA2 | 0\|2\|2 | 4 | 1.33 | 0\|2\|2 | 0.01 | 0.01 | 0.01 | 0.01 | 0.01 | −7.85 | 1 | 0.8 |
| O95831-3 | AIFM1 | 3\|2\|2 | 7 | 2.33 | 2\|0\|3 | 0.01 | 0.01 | 0.01 | 0.01 | 0.01 | −5.71 | 1.4 | 0.8 |
| Q9H089 | LSG1 | 2\|0\|2 | 4 | 1.33 | 0\|2\|2 | 0.01 | 0.01 | 0.01 | 0.01 | 0.01 | −7.85 | 1 | 0.8 |
| Q9ULC3 | RAB23 | 3\|0\|0 | 3 | 1 | 0\|2\|2 | 0.01 | 0.04 | 0.01 | 0.04 | 0.01 | −7.85 | 0.75 | 0.78 |
| O43237-2 | DYNC1LI2 | 2\|0\|4 | 6 | 2 | 0\|3\|3 | 0.01 | 0.02 | 0.01 | 0.02 | 0.01 | −10.39 | 1 | 0.81 |
| Q15019 | 2-Sep | 3\|0\|0 | 3 | 1 | 2\|0\|2 | 0.01 | 0.04 | 0.01 | 0.04 | 0.01 | −7.85 | 0.75 | 0.78 |
| O14929-2 | HAT1 | 4\|2\|0 | 6 | 2 | 2\|2\|2 | 0.01 | 0.02 | 0.01 | 0.02 | 0.01 | −10.49 | 1 | 0.81 |
| P06753-3 | TPM3 | 2\|0\|2 | 4 | 1.33 | 2\|0\|2 | 0.01 | 0.01 | 0.01 | 0.01 | 0.01 | −7.85 | 1 | 0.8 |
| Q99570 | PIK3R4 | 0\|2\|2 | 4 | 1.33 | 0\|2\|2 | 0.01 | 0.01 | 0.01 | 0.01 | 0.01 | −7.85 | 1 | 0.8 |
| Q9UNF1-2 | MAGED2 | 4\|5\|7 | 16 | 5.33 | 4\|2\|3 | 0.01 | 0.03 | 0.01 | 0.03 | 0.01 | −7.59 | 1.78 | 0.79 |
| P11166 | SLC2A1 | 4\|3\|6 | 13 | 4.33 | 4\|2\|2 | 0.01 | 0.02 | 0.01 | 0.02 | 0.01 | −7.86 | 1.62 | 0.79 |
| Q8N6T3 | ARFGAP1 | 4\|4\|4 | 12 | 4 | 0\|3\|4 | 0.01 | 0.01 | 0.01 | 0.01 | 0.01 | −5.11 | 1.71 | 0.8 |
| P04049 | RAF1 | 4\|2\|2 | 8 | 2.67 | 2\|0\|4 | 0.01 | 0.02 | 0.01 | 0.02 | 0.01 | −6.72 | 1.33 | 0.8 |
| P30101 | PDIA3 | 2\|2\|2 | 6 | 2 | 0\|2\|2 | 0.01 | 0.01 | 0.01 | 0.01 | 0.01 | −4.72 | 1.5 | 0.79 |
| Q13637 | RAB32 | 2\|4\|3 | 9 | 3 | 3\|3\|0 | 0.01 | 0.02 | 0.01 | 0.02 | 0.01 | −6.87 | 1.5 | 0.79 |
| O95487-2 | SEC24B | 2\|2\|3 | 7 | 2.33 | 3\|0\|2 | 0.01 | 0.01 | 0.01 | 0.01 | 0.01 | −5.71 | 1.4 | 0.8 |
| Q13470-2 | TNK1 | 15\|18\|13 | 46 | 15.33 | 9\|8\|5 | 0.01 | 0.03 | 0.01 | 0.03 | 0.01 | −10.07 | 2.09 | 0.79 |
| O43847 | NRDC | 0\|2\|0 | 2 | 0.67 | 0\|4\|0 | 0.01 | 0.02 | 0.01 | 0.02 | 0.01 | −7.33 | 0.5 | 0.8 |
| Q8IZH2-2 | XRN1 | 3\|0\|2 | 5 | 1.67 | 0\|2\|3 | 0.01 | 0.01 | 0.01 | 0.01 | 0.01 | −9.18 | 1 | 0.8 |
| O43159 | RRP8 | 0\|0\|3 | 3 | 1 | 2\|0\|2 | 0.01 | 0.04 | 0.01 | 0.04 | 0.01 | −7.85 | 0.75 | 0.78 |
| Q14432 | PDE3A | 7\|6\|4 | 17 | 5.67 | 2\|3\|4 | 0.01 | 0.03 | 0.01 | 0.03 | 0.01 | −7.59 | 1.89 | 0.78 |
| P12931 | SRC | 2\|2\|2 | 6 | 2 | 2\|2\|0 | 0.01 | 0.01 | 0.01 | 0.01 | 0.01 | −4.72 | 1.5 | 0.79 |
| Q96HC4-6 | PDLIM5 | 9\|5\|10 | 24 | 8 | 4\|5\|4 | 0.01 | 0.02 | 0.01 | 0.02 | 0.01 | −10.83 | 1.85 | 0.79 |
| P48729 | CSNK1A1 | 3\|5\|3 | 11 | 3.67 | 4\|0\|3 | 0.01 | 0.02 | 0.01 | 0.02 | 0.01 | −6.46 | 1.57 | 0.79 |
| Q8NBF2-2 | NHLRC2 | 2\|0\|2 | 4 | 1.33 | 2\|0\|2 | 0.01 | 0.01 | 0.01 | 0.01 | 0.01 | −7.85 | 1 | 0.8 |
| Q96A65 | EXOC4 | 3\|3\|3 | 9 | 3 | 2\|3\|0 | 0.01 | 0.01 | 0.01 | 0.01 | 0.01 | −4.3 | 1.8 | 0.78 |
| Q96MU7-2 | YTHDC1 | 0\|3\|4 | 7 | 2.33 | 3\|3\|0 | 0.01 | 0.02 | 0.01 | 0.02 | 0.01 | −10.39 | 1.17 | 0.79 |
| Q9Y3D8 | AK6 | 2\|3\|3 | 8 | 2.67 | 3\|2\|0 | 0.01 | 0.01 | 0.01 | 0.01 | 0.01 | −5.71 | 1.6 | 0.79 |
| P27707 | DCK | 4\|0\|2 | 6 | 2 | 2\|2\|2 | 0.01 | 0.02 | 0.01 | 0.02 | 0.01 | −10.49 | 1 | 0.81 |
| Q6YN16-2 | HSDL2 | 5\|3\|3 | 11 | 3.67 | 3\|0\|4 | 0.01 | 0.02 | 0.01 | 0.02 | 0.01 | −6.46 | 1.57 | 0.79 |
| O60427 | FADS1 | 0\|2\|2 | 4 | 1.33 | 0\|2\|2 | 0.01 | 0.01 | 0.01 | 0.01 | 0.01 | −7.85 | 1 | 0.8 |
| Q9UJZ1-2 | STOML2 | 2\|0\|2 | 4 | 1.33 | 2\|0\|2 | 0.01 | 0.01 | 0.01 | 0.01 | 0.01 | −7.85 | 1 | 0.8 |
| Q96G03 | PGM2 | 2\|2\|3 | 7 | 2.33 | 3\|0\|2 | 0.01 | 0.01 | 0.01 | 0.01 | 0.01 | −5.71 | 1.4 | 0.8 |
| Q9H3S7 | PTPN23 | 2\|0\|2 | 4 | 1.33 | 0\|2\|2 | 0.01 | 0.01 | 0.01 | 0.01 | 0.01 | −7.85 | 1 | 0.8 |
| Q9UNY4 | TTF2 | 4\|2\|5 | 11 | 3.67 | 3\|4\|0 | 0.01 | 0.02 | 0.01 | 0.02 | 0.01 | −7.98 | 1.57 | 0.79 |
| Q15056-2 | EIF4H | 3\|2\|2 | 7 | 2.33 | 0\|3\|2 | 0.01 | 0.01 | 0.01 | 0.01 | 0.01 | −5.71 | 1.4 | 0.8 |
| Q12906 | ILF3 | 21\|14\|19 | 54 | 18 | 29\|37\|29 | 0 | 0 | 0 | 0 | 0 | −99.68 | 0.57 | 0.92 |
| P40227 | CCT6A | 31\|30\|35 | 96 | 32 | 33\|35\|44 | 0 | 0 | 0 | 0 | 0 | −91.49 | 0.86 | 0.92 |
| P24941-2 | CDK2 | 8\|3\|7 | 18 | 6 | 5\|4\|6 | 0 | 0 | 0 | 0 | 0 | −16.28 | 1.2 | 0.87 |
| P16435 | POR | 7\|5\|6 | 18 | 6 | 9\|3\|8 | 0 | 0 | 0 | 0 | 0 | −19.06 | 0.9 | 0.9 |
| P84077 | ARF1 | 14\|15\|12 | 41 | 13.67 | 18\|14\|16 | 0 | 0 | 0 | 0 | 0 | −42.17 | 0.85 | 0.92 |
| P62826 | RAN | 23\|13\|13 | 49 | 16.33 | 18\|15\|18 | 0 | 0 | 0 | 0 | 0 | −44.22 | 0.96 | 0.91 |
| P51610-2 | HCFC1 | 2\|0\|2 | 4 | 1.33 | 12\|5\|4 | 0 | 0 | 0 | 0 | 0 | −32 | 0.19 | 0.92 |
| P51610-4 | HCFC1 | 3\|0\|2 | 5 | 1.67 | 13\|5\|5 | 0 | 0 | 0 | 0 | 0 | −35.01 | 0.22 | 0.92 |
| Q00535 | CDK5 | 5\|4\|4 | 13 | 4.33 | 4\|4\|3 | 0 | 0 | 0 | 0 | 0 | −9.93 | 1.18 | 0.87 |
| Q6Y7W6-4 | GIGYF2 | 6\|6\|10 | 22 | 7.33 | 5\|11\|9 | 0 | 0 | 0 | 0 | 0 | −23.72 | 0.88 | 0.9 |
| O00763-3 | ACACB | 129\|117\|127 | 373 | 124.33 | 116\|118\| | 0 | 0 | 0 | 0 | 0 | −233.73 | 1.09 | 0.92 |
| | | | | | 107 | | | | | | | | |
| Q9UMZ2-8 | SYNRG | 12\|8\|10 | 30 | 10 | 11\|9\|7 | 0 | 0 | 0 | 0 | 0 | −22.92 | 1.11 | 0.9 |
| Q12873-2 | CHD3 | 0\|0\|2 | 2 | 0.67 | 3\|4\|5 | 0 | 0 | 0 | 0 | 0 | −19.16 | 0.17 | 0.92 |
| Q9UBM7 | DHCR7 | 2\|0\|0 | 2 | 0.67 | 0\|3\|2 | 0 | 0 | 0 | 0 | 0 | −9.18 | 0.4 | 0.84 |
| P02545 | LMNA | 14\|19\|18 | 51 | 17 | 17\|16\|20 | 0 | 0 | 0 | 0 | 0 | −45.05 | 0.96 | 0.91 |
| P40429 | RPL13A | 7\|5\|11 | 23 | 7.67 | 9\|10\|8 | 0 | 0 | 0 | 0 | 0 | −28 | 0.85 | 0.91 |
| Q7Z3T8 | ZFYVE16 | 8\|6\|5 | 19 | 6.33 | 11\|3\|9 | 0 | 0 | 0 | 0 | 0 | −22.63 | 0.83 | 0.9 |
| Q13838 | DDX39B | 14\|13\|10 | 37 | 12.33 | 11\|10\|12 | 0 | 0 | 0 | 0 | 0 | −27.07 | 1.12 | 0.91 |
| Q96T37-4 | RBM15 | 2\|0\|2 | 4 | 1.33 | 5\|2\|3 | 0 | 0 | 0 | 0 | 0 | −16.29 | 0.4 | 0.92 |
| Q12834 | CDC20 | 3\|3\|8 | 14 | 4.67 | 4\|4\|3 | 0 | 0.01 | 0 | 0.01 | 0 | −11.41 | 1.27 | 0.81 |
| P02769 | P02769 | 21\|14\|13 | 48 | 16 | 39\|5\|6 | 0 | 0 | 0 | 0 | 0 | −41.58 | 0.96 | 0.91 |
| P02768 | ALB | 3\|2\|2 | 7 | 2.33 | 4\|2\|2 | 0 | 0 | 0 | 0 | 0 | −9.34 | 0.88 | 0.87 |
| P05387 | RPLP2 | 2\|4\|5 | 11 | 3.67 | 3\|3\|3 | 0 | 0 | 0 | 0 | 0 | −10.47 | 1.22 | 0.85 |
| P78346 | RPP30 | 0\|2\|0 | 2 | 0.67 | 0\|3\|2 | 0 | 0 | 0 | 0 | 0 | −9.18 | 0.4 | 0.84 |
| P05388 | RPLPO | 2\|6\|6 | 14 | 4.67 | 5\|5\|3 | 0 | 0 | 0 | 0 | 0 | −15.6 | 1.08 | 0.87 |
| P62979 | RPS27A | 26\|20\|28 | 74 | 24.67 | 25\|22\|21 | 0 | 0 | 0 | 0 | 0 | −53.79 | 1.09 | 0.92 |
| Q9BQ52-4 | ELAC2 | 3\|2\|0 | 5 | 1.67 | 2\|3\|3 | 0 | 0 | 0 | 0 | 0 | −13.44 | 0.63 | 0.87 |
| P35251-2 | RFC1 | 0\|4\|3 | 7 | 2.33 | 5\|4\|4 | 0 | 0 | 0 | 0 | 0 | −20.66 | 0.54 | 0.92 |
| Q15365 | PCBP1 | 24\|21\|30 | 75 | 25 | 24\|24\|24 | 0 | 0 | 0 | 0 | 0 | −57.05 | 1.04 | 0.92 |
| Q13085-4 | ACACA | 734\|656\|730 | 2120 | 706.67 | 657\|672\| | 0 | 0 | 0 | 0 | 0 | #NAME? | 1.06 | 0.92 |
| | | | | | 672 | | | | | | | | |
| P29401 | TKT | 5\|2\|4 | 11 | 3.67 | 3\|4\|3 | 0 | 0 | 0 | 0 | 0 | −11.76 | 1.1 | 0.86 |
| Q13907 | IDI1 | 4\|5\|3 | 12 | 4 | 5\|6\|4 | 0 | 0 | 0 | 0 | 0 | −16.28 | 0.8 | 0.92 |
| P84085 | ARF5 | 7\|9\|5 | 21 | 7 | 10\|6\|10 | 0 | 0 | 0 | 0 | 0 | −26.73 | 0.81 | 0.91 |
| P07900 | HSP90AA1 | 50\|42\|51 | 143 | 47.67 | 56\|55\|55 | 0 | 0 | 0 | 0 | 0 | −138.42 | 0.86 | 0.92 |
| P39023 | RPL3 | 26\|21\|18 | 65 | 21.67 | 23\|30\|25 | 0 | 0 | 0 | 0 | 0 | −69.46 | 0.83 | 0.92 |
| P35241 | RDX | 0\|2\|0 | 2 | 0.67 | 3\|3\|2 | 0 | 0 | 0 | 0 | 0 | −13.44 | 0.25 | 0.92 |
| O75439 | PMPCB | 8\|4\|6 | 18 | 6 | 9\|9\|11 | 0 | 0 | 0 | 0 | 0 | −32.71 | 0.62 | 0.92 |
| O95251-2 | KAT7 | 0\|0\|2 | 2 | 0.67 | 2\|3\|2 | 0 | 0 | 0 | 0 | 0 | −11.98 | 0.29 | 0.92 |
| Q96AC1 | FERMT2 | 17\|15\|13 | 45 | 15 | 9\|10\|14 | 0 | 0 | 0 | 0 | 0 | −22.74 | 1.36 | 0.9 |
| P10316 | HLA-A | 4\|2\|2 | 8 | 2.67 | 5\|3\|3 | 0 | 0 | 0 | 0 | 0 | −13.04 | 0.73 | 0.88 |
| P13646-3 | KRT13 | 10\|7\|6 | 23 | 7.67 | 9\|8\|6 | 0 | 0 | 0 | 0 | 0 | −21.22 | 1 | 0.9 |
| Q9H1A4 | ANAPC1 | 2\|0\|0 | 2 | 0.67 | 6\|6\|5 | 0 | 0 | 0 | 0 | 0 | −26.53 | 0.12 | 0.92 |
| Q7Z6Z7-3 | HUWE1 | 5\|4\|4 | 13 | 4.33 | 4\|7\|11 | 0 | 0 | 0 | 0 | 0 | −23.3 | 0.59 | 0.92 |
| Q99081 | TCF12 | 0\|2\|0 | 2 | 0.67 | 7\|3\|7 | 0 | 0 | 0 | 0 | 0 | −26.53 | 0.12 | 0.92 |
| P60981-2 | DSTN | 0\|0\|7 | 7 | 2.33 | 3\|4\|6 | 0 | 0 | 0 | 0 | 0 | −20.66 | 0.54 | 0.87 |
| Q5TDH0 | DDI2 | 4\|3\|2 | 9 | 3 | 5\|2\|2 | 0 | 0 | 0 | 0 | 0 | −10.47 | 1 | 0.86 |
| Q14562 | DHX8 | 2\|0\|0 | 2 | 0.67 | 0\|6\|4 | 0 | 0 | 0 | 0 | 0 | −15.92 | 0.2 | 0.92 |
| O60763 | USO1 | 3\|2\|4 | 9 | 3 | 5\|3\|3 | 0 | 0 | 0 | 0 | 0 | −13.04 | 0.82 | 0.88 |
| P18077 | RPL35A | 5\|3\|4 | 12 | 4 | 6\|5\|5 | 0 | 0 | 0 | 0 | 0 | −17.58 | 0.75 | 0.92 |
| Q15181 | PPA1 | 6\|4\|6 | 16 | 5.33 | 7\|7\|9 | 0 | 0 | 0 | 0 | 0 | −24.75 | 0.7 | 0.92 |
| Q04637-8 | EIF4G1 | 10\|12\|13 | 35 | 11.67 | 14\|16\|13 | 0 | 0 | 0 | 0 | 0 | −39.35 | 0.81 | 0.92 |
| P17844 | DDX5 | 75\|76\|71 | 222 | 74 | 72\|66\|76 | 0 | 0 | 0 | 0 | 0 | −151.24 | 1.04 | 0.92 |
| Q2TAY7 | SMU1 | 6\|4\|6 | 16 | 5.33 | 7\|3\|6 | 0 | 0 | 0 | 0 | 0 | −15.87 | 1 | 0.89 |
| Q9Y5A9-2 | YTHDF2 | 2\|2\|0 | 4 | 1.33 | 3\|2\|2 | 0 | 0 | 0 | 0 | 0 | −11.98 | 0.57 | 0.92 |
| 043242 | PSMD3 | 12\|8\|11 | 31 | 10.33 | 9\|15\|14 | 0 | 0 | 0 | 0 | 0 | −36.58 | 0.82 | 0.92 |
| Q8N3C0 | ASCC3 | 13\|11\|10 | 34 | 11.33 | 8\|11\|10 | 0 | 0 | 0 | 0 | 0 | −22.38 | 1.17 | 0.9 |
| Q9NRF8 | CTPS2 | 0\|0\|2 | 2 | 0.67 | 2\|3\|2 | 0 | 0 | 0 | 0 | 0 | −11.98 | 0.29 | 0.92 |
| Q8WU90 | ZC3H15 | 0\|2\|2 | 4 | 1.33 | 3\|2\|0 | 0 | 0 | 0 | 0 | 0 | −9.18 | 0.8 | 0.83 |
| Q9BUF5 | TUBB6 | 39\|38\|38 | 115 | 38.33 | 41\|33\|34 | 0 | 0 | 0 | 0 | 0 | −74.58 | 1.06 | 0.92 |
| P48960-2 | CD97 | 2\|3\|3 | 8 | 2.67 | 6\|2\|5 | 0 | 0 | 0 | 0 | 0 | −15.6 | 0.62 | 0.92 |
| Q562R1 | ACTBL2 | 23\|26\|34 | 83 | 27.67 | 47\|36\|37 | 0 | 0 | 0 | 0 | 0 | −114.22 | 0.69 | 0.92 |
| Q02878 | RPL6 | 20\|20\|18 | 58 | 19.33 | 26\|21\|23 | 0 | 0 | 0 | 0 | 0 | −59.44 | 0.83 | 0.92 |
| P16152 | CBR1 | 2\|3\|2 | 7 | 2.33 | 8\|6\|5 | 0 | 0 | 0 | 0 | 0 | −23.65 | 0.37 | 0.92 |
| Q9UNX3 | RPL26L1 | 16\|11\|12 | 39 | 13 | 15\|11\|13 | 0 | 0 | 0 | 0 | 0 | −32.74 | 1 | 0.91 |
| Q9UGP8 | SEC63 | 0\|0\|2 | 2 | 0.67 | 3\|2\|2 | 0 | 0 | 0 | 0 | 0 | −11.98 | 0.29 | 0.92 |
| O43776 | NARS | 12\|12\|13 | 37 | 12.33 | 12\|16\|11 | 0 | 0 | 0 | 0 | 0 | −31.22 | 0.95 | 0.92 |
| P26640 | VARS | 20\|18\|21 | 59 | 19.67 | 16\|19\|20 | 0 | 0 | 0 | 0 | 0 | −41.32 | 1.07 | 0.91 |
| P26641 | EEF1G | 13\|11\|15 | 39 | 13 | 16\|16\|16 | 0 | 0 | 0 | 0 | 0 | −43.89 | 0.81 | 0.92 |
| Q16527 | CSRP2 | 16\|10\|10 | 36 | 12 | 10\|9\|9 | 0 | 0 | 0 | 0 | 0 | −21.2 | 1.29 | 0.89 |
| P29692-3 | EEF1D | 8\|10\|9 | 27 | 9 | 7\|8\|8 | 0 | 0 | 0 | 0 | 0 | −18.23 | 1.17 | 0.9 |
| P60709 | ACTB | 90\|85\|91 | 266 | 88.67 | 109\|107\|97 | 0 | 0 | 0 | 0 | 0 | −248.92 | 0.85 | 0.92 |
| Q00534 | CDK6 | 9\|6\|9 | 24 | 8 | 8\|7\|7 | 0 | 0 | 0 | 0 | 0 | −20 | 1.09 | 0.9 |
| P58107 | EPPK1 | 24\|28\|22 | 74 | 24.67 | 32\|38\|32 | 0 | 0 | 0 | 0 | 0 | −92.81 | 0.73 | 0.92 |
| O00203-3 | AP3B1 | 0\|0\|3 | 3 | 1 | 3\|3\|3 | 0 | 0 | 0 | 0 | 0 | −14.8 | 0.33 | 0.92 |
| Q9UHB6-5 | LIMA1 | 10\|11\|8 | 29 | 9.67 | 5\|5\|6 | 0 | 0 | 0 | 0 | 0 | −10.07 | 1.81 | 0.84 |
| Q9UHB6-2 | LIMA1 | 10\|11\|10 | 31 | 10.33 | 6\|5\|7 | 0 | 0 | 0 | 0 | 0 | −9.61 | 1.72 | 0.87 |
| Q9NUU7 | DDX19A | 8\|6\|7 | 21 | 7 | 8\|3\|8 | 0 | 0 | 0 | 0 | 0 | −16.41 | 1.11 | 0.9 |
| P49411 | TUFM | 23\|29\|29 | 81 | 27 | 33\|26\|27 | 0 | 0 | 0 | 0 | 0 | −70.94 | 0.94 | 0.92 |
| P48147 | PREP | 3\|5\|3 | 11 | 3.67 | 7\|3\|4 | 0 | 0 | 0 | 0 | 0 | −15.06 | 0.79 | 0.89 |
| P62495 | ETF1 | 3\|4\|3 | 10 | 3.33 | 3\|3\|3 | 0 | 0 | 0 | 0 | 0 | −9.04 | 1.11 | 0.86 |
| Q14966 | ZNF638 | 3\|4\|0 | 7 | 2.33 | 9\|7\|4 | 0 | 0 | 0 | 0 | 0 | −30.95 | 0.35 | 0.92 |
| P50395 | GDI2 | 15\|15\|17 | 47 | 15.67 | 16\|19\|19 | 0 | 0 | 0 | 0 | 0 | −44.65 | 0.87 | 0.92 |
| P49841-2 | GSK3B | 6\|3\|4 | 13 | 4.33 | 4\|6\|3 | 0 | 0 | 0 | 0 | 0 | −13.81 | 1 | 0.88 |
| Q13643 | FHL3 | 2\|2\|2 | 6 | 2 | 3\|2\|2 | 0 | 0 | 0 | 0 | 0 | −8.15 | 0.86 | 0.92 |
| P25205 | MCM3 | 3\|2\|2 | 7 | 2.33 | 3\|2\|2 | 0 | 0 | 0 | 0 | 0 | −8.15 | 1 | 0.85 |
| Q16186 | ADRM1 | 4\|4\|4 | 12 | 4 | 3\|2\|6 | 0 | 0 | 0 | 0 | 0 | −9.95 | 1.09 | 0.88 |
| P0C0S5 | H2AFZ | 0\|0\|2 | 2 | 0.67 | 2\|2\|2 | 0 | 0 | 0 | 0 | 0 | −10.49 | 0.33 | 0.92 |
| O95453-4 | PARN | 3\|2\|0 | 5 | 1.67 | 2\|2\|2 | 0 | 0 | 0 | 0 | 0 | −10.49 | 0.83 | 0.84 |
| A0MZ66 | SHTN1 | 11\|12\|8 | 31 | 10.33 | 19\|19\|21 | 0 | 0 | 0 | 0 | 0 | −64.37 | 0.53 | 0.92 |
| Q96HS1 | PGAM5 | 3\|4\|4 | 11 | 3.67 | 6\|4\|3 | 0 | 0 | 0 | 0 | 0 | −13.81 | 0.85 | 0.92 |
| Q7Z478 | DHX29 | 5\|3\|4 | 12 | 4 | 3\|3\|2 | 0 | 0.01 | 0 | 0.01 | 0 | −7.86 | 1.5 | 0.83 |
| Q9NVM9 | INTS13 | 0\|0\|3 | 3 | 1 | 4\|2\|2 | 0 | 0 | 0 | 0 | 0 | −13.44 | 0.38 | 0.87 |
| Q99613-2 | EIF3C | 13\|12\|19 | 44 | 14.67 | 17\|17\|13 | 0 | 0 | 0 | 0 | 0 | −40.94 | 0.94 | 0.91 |
| Q12797-10 | ASPH | 3\|4\|3 | 10 | 3.33 | 3\|3\|2 | 0 | 0 | 0 | 0 | 0 | −7.86 | 1.25 | 0.85 |
| O15067 | PFAS | 23\|22\|20 | 65 | 21.67 | 21\|21\|25 | 0 | 0 | 0 | 0 | 0 | −52.58 | 0.97 | 0.92 |
| Q9NSV4-7 | DIAPH3 | 0\|0\|2 | 2 | 0.67 | 0\|2\|2 | 0 | 0.01 | 0 | 0.01 | 0 | −7.85 | 0.5 | 0.82 |
| P60891 | PRPS1 | 9\|7\|9 | 25 | 8.33 | 6\|10\|8 | 0 | 0 | 0 | 0 | 0 | −20.85 | 1.04 | 0.9 |
| Q9NZM5 | NOP53 | 2\|0\|0 | 2 | 0.67 | 0\|2\|2 | 0 | 0.01 | 0 | 0.01 | 0 | −7.85 | 0.5 | 0.82 |
| P31942-2 | HNRNPH3 | 6\|3\|4 | 13 | 4.33 | 4\|5\|4 | 0 | 0 | 0 | 0 | 0 | −13.81 | 1 | 0.88 |
| Q86YP4-2 | GATAD2A | 6\|4\|4 | 14 | 4.67 | 7\|5\|5 | 0 | 0 | 0 | 0 | 0 | −17.11 | 0.82 | 0.9 |
| Q9BXP5-5 | SRRT | 5\|2\|2 | 9 | 3 | 4\|4\|4 | 0 | 0 | 0 | 0 | 0 | −14.26 | 0.75 | 0.88 |
| P42765 | ACAA2 | 12\|12\|9 | 33 | 11 | 3\|6\|9 | 0 | 0 | 0 | 0 | 0 | −10.95 | 1.83 | 0.85 |
| P06748-2 | NPM1 | 6\|6\|6 | 18 | 6 | 9\|8\|9 | 0 | 0 | 0 | 0 | 0 | −24.93 | 0.69 | 0.92 |
| Q96H79 | ZC3HAV1L | 0\|2\|0 | 2 | 0.67 | 0\|2\|2 | 0 | 0.01 | 0 | 0.01 | 0 | −7.85 | 0.5 | 0.82 |
| Q9Y295 | DRG1 | 5\|8\|4 | 17 | 5.67 | 10\|4\|4 | 0 | 0 | 0 | 0 | 0 | −18.07 | 0.94 | 0.89 |
| Q6UXN9 | WDR82 | 5\|6\|5 | 16 | 5.33 | 4\|6\|4 | 0 | 0 | 0 | 0 | 0 | −12.01 | 1.14 | 0.89 |
| P17066 | HSPA6 | 12\|11\|11 | 34 | 11.33 | 13\|9\|12 | 0 | 0 | 0 | 0 | 0 | −26.81 | 1 | 0.91 |
| P49959 | MRE11 | 3\|3\|3 | 9 | 3 | 6\|5\|4 | 0 | 0 | 0 | 0 | 0 | −16.28 | 0.6 | 0.92 |
| P68371 | TUBB4B | 104\|104\|104 | 312 | 104 | 114\|111\| | 0 | 0 | 0 | 0 | 0 | −244.53 | 0.93 | 0.92 |
| | | | | | 109 | | | | | | | | |
| Q01813 | PFKP | 18\|27\|23 | 68 | 22.67 | 23\|22\|21 | 0 | 0 | 0 | 0 | 0 | −54.5 | 1.03 | 0.92 |
| P49792 | RANBP2 | 35\|36\|47 | 118 | 39.33 | 64\|55\|55 | 0 | 0 | 0 | 0 | 0 | −161.27 | 0.68 | 0.92 |
| P56545 | CTBP2 | 4\|2\|2 | 8 | 2.67 | 4\|2\|5 | 0 | 0 | 0 | 0 | 0 | −13.04 | 0.73 | 0.88 |
| P23588 | EIF4B | 7\|8\|9 | 24 | 8 | 10\|8\|7 | 0 | 0 | 0 | 0 | 0 | −22.07 | 0.96 | 0.91 |
| O60293-2 | ZFC3H1 | 2\|3\|2 | 7 | 2.33 | 0\|3\|4 | 0 | 0 | 0 | 0 | 0 | −7.98 | 1 | 0.85 |
| O76031 | CLPX | 4\|6\|5 | 15 | 5 | 7\|5\|4 | 0 | 0 | 0 | 0 | 0 | −15.87 | 0.94 | 0.9 |
| Q9NRZ9-3 | HELLS | 6\|4\|3 | 13 | 4.33 | 51\|50\|52 | 0 | 0 | 0 | 0 | 0 | −215.19 | 0.08 | 0.92 |
| Q86W42-3 | THOC6 | 0\|4\|2 | 6 | 2 | 4\|4\|4 | 0 | 0 | 0 | 0 | 0 | −19.16 | 0.5 | 0.92 |
| P18124 | RPL7 | 20\|13\|18 | 51 | 17 | 24\|13\|15 | 0 | 0 | 0 | 0 | 0 | −45.24 | 0.98 | 0.91 |
| O00154-6 | ACOT7 | 5\|5\|5 | 15 | 5 | 8\|5\|4 | 0 | 0 | 0 | 0 | 0 | −15.55 | 0.88 | 0.92 |
| P62873-2 | GNB1 | 3\|3\|4 | 10 | 3.33 | 2\|3\|5 | 0 | 0 | 0 | 0 | 0 | −10.22 | 1 | 0.88 |
| P31483 | TIA1 | 7\|5\|4 | 16 | 5.33 | 4\|4\|4 | 0 | 0 | 0 | 0 | 0 | −11.11 | 1.33 | 0.86 |
| P26368-2 | U2AF2 | 12\|7\|9 | 28 | 9.33 | 20\|9\|7 | 0 | 0 | 0 | 0 | 0 | −35.25 | 0.78 | 0.92 |
| P20340 | RAB6A | 8\|8\|8 | 24 | 8 | 9\|10\|11 | 0 | 0 | 0 | 0 | 0 | −26.54 | 0.8 | 0.92 |
| O00505 | KPNA3 | 2\|2\|2 | 6 | 2 | 3\|3\|2 | 0 | 0 | 0 | 0 | 0 | −9.34 | 0.75 | 0.92 |
| Q9Y4P3 | TBL2 | 6\|7\|5 | 18 | 6 | 4\|6\|4 | 0 | 0 | 0 | 0 | 0 | −12.01 | 1.29 | 0.88 |
| P08708 | RPS17 | 5\|6\|5 | 16 | 5.33 | 3\|8\|11 | 0 | 0 | 0 | 0 | 0 | −21.35 | 0.73 | 0.92 |
| Q01581 | HMGCS1 | 12\|13\|15 | 40 | 13.33 | 17\|19\|22 | 0 | 0 | 0 | 0 | 0 | −54.84 | 0.69 | 0.92 |
| P29692 | EEF1D | 9\|12\|11 | 32 | 10.67 | 7\|8\|10 | 0 | 0 | 0 | 0 | 0 | −19.13 | 1.28 | 0.9 |
| P78344 | EIF4G2 | 26\|24\|28 | 78 | 26 | 21\|11\|14 | 0 | 0 | 0 | 0 | 0 | −22.65 | 1.7 | 0.9 |
| Q9H2U1-3 | DHX36 | 5\|5\|5 | 15 | 5 | 4\|3\|3 | 0 | 0 | 0 | 0 | 0 | −7.34 | 1.5 | 0.85 |
| Q9BXB5 | OSBPL10 | 0\|0\|2 | 2 | 0.67 | 2\|3\|2 | 0 | 0 | 0 | 0 | 0 | −11.98 | 0.29 | 0.92 |
| Q96P48-7 | ARAP1 | 0\|0\|2 | 2 | 0.67 | 3\|0\|2 | 0 | 0 | 0 | 0 | 0 | −9.18 | 0.4 | 0.84 |
| Q14566 | MCM6 | 14\|8\|12 | 34 | 11.33 | 8\|13\|16 | 0 | 0 | 0 | 0 | 0 | −35.26 | 0.92 | 0.91 |
| O00139-2 | KIF2A | 4\|3\|5 | 12 | 4 | 3\|5\|5 | 0 | 0 | 0 | 0 | 0 | −13.81 | 0.92 | 0.89 |
| Q15637-4 | SF1 | 2\|3\|2 | 7 | 2.33 | 2\|2\|3 | 0 | 0 | 0 | 0 | 0 | −8.15 | 1 | 0.85 |
| Q9BSJ8 | ESYT1 | 2\|2\|0 | 4 | 1.33 | 2\|2\|2 | 0 | 0 | 0 | 0 | 0 | −10.49 | 0.67 | 0.92 |
| P62701 | RPS4X | 35\|35\|43 | 113 | 37.67 | 39\|41\|36 | 0 | 0 | 0 | 0 | 0 | −88.44 | 0.97 | 0.92 |
| P68431 | HIST1H3G; | 2\|2\|2 | 6 | 2 | 3\|2\|2 | 0 | 0 | 0 | 0 | 0 | −8.15 | 0.86 | 0.92 |
| | HIST1H3C; | | | | | | | | | | | | |
| | HIST [symbol] | | | | | | | | | | | | |
| P17858 | PFKL | 6\|7\|4 | 17 | 5.67 | 6\|7\|8 | 0 | 0 | 0 | 0 | 0 | −22.13 | 0.81 | 0.92 |
| Q9BTC0 | DIDO1 | 0\|0\|2 | 2 | 0.67 | 6\|4\|4 | 0 | 0 | 0 | 0 | 0 | −22.13 | 0.14 | 0.92 |
| O75083 | WDR1 | 18\|18\|16 | 52 | 17.33 | 19\|19\|14 | 0 | 0 | 0 | 0 | 0 | −40.71 | 1 | 0.91 |
| P58876 | HIST1H2BD | 3\|4\|6 | 13 | 4.33 | 9\|8\|5 | 0 | 0 | 0 | 0 | 0 | −25.47 | 0.59 | 0.92 |
| Q15149-7 | PLEC | 78\|76\|81 | 235 | 78.33 | 66\|62\|65 | 0 | 0 | 0 | 0 | 0 | −119.49 | 1.22 | 0.92 |
| P62913-2 | RPL11 | 7\|3\|4 | 14 | 4.67 | 3\|4\|4 | 0 | 0 | 0 | 0 | 0 | −11.41 | 1.27 | 0.84 |
| P53350 | PLK1 | 4\|3\|4 | 11 | 3.67 | 3\|3\|5 | 0 | 0 | 0 | 0 | 0 | −11.41 | 1 | 0.88 |
| P54886 | ALDH18A1 | 18\|16\|19 | 53 | 17.67 | 21\|16\|18 | 0 | 0 | 0 | 0 | 0 | −44.3 | 0.96 | 0.91 |
| P52732 | KIF11 | 10\|7\|11 | 28 | 9.33 | 10\|11\|9 | 0 | 0 | 0 | 0 | 0 | −28.2 | 0.93 | 0.91 |
| P46781 | RPS9 | 17\|16\|14 | 47 | 15.67 | 14\|16\|17 | 0 | 0 | 0 | 0 | 0 | −37.76 | 1 | 0.91 |
| P13804-2 | ETFA | 5\|6\|5 | 16 | 5.33 | 5\|7\|7 | 0 | 0 | 0 | 0 | 0 | −17.93 | 0.84 | 0.92 |
| O94808 | GFPT2 | 2\|2\|2 | 6 | 2 | 3\|2\|3 | 0 | 0 | 0 | 0 | 0 | −9.34 | 0.75 | 0.92 |
| P11182 | DBT | 67\|61\|76 | 204 | 68 | 59\|65\|71 | 0 | 0 | 0 | 0 | 0 | −143.5 | 1.05 | 0.92 |
| Q99459 | CDC5L | 3\|2\|0 | 5 | 1.67 | 7\|5\|5 | 0 | 0 | 0 | 0 | 0 | −26.53 | 0.29 | 0.92 |
| Q16512 | PKN1 | 0\|0\|2 | 2 | 0.67 | 4\|5\|5 | 0 | 0 | 0 | 0 | 0 | −22.13 | 0.14 | 0.92 |
| O15143 | ARPC1B | 12\|12\|8 | 32 | 10.67 | 11\|9\|11 | 0 | 0 | 0 | 0 | 0 | −27.78 | 1.03 | 0.91 |
| P20020-5 | ATP2B1 | 2\|2\|2 | 6 | 2 | 0\|2\|4 | 0 | 0 | 0 | 0 | 0 | −6.72 | 1 | 0.92 |
| P20340-2 | RAB6A | 8\|8\|9 | 25 | 8.33 | 11\|11\|12 | 0 | 0 | 0 | 0 | 0 | −31.51 | 0.74 | 0.92 |
| P40939 | HADHA | 17\|16\|17 | 50 | 16.67 | 16\|14\|19 | 0 | 0 | 0 | 0 | 0 | −37.17 | 1.02 | 0.91 |
| A0A0B4J2 | LOC- | 0\|0\|2 | 2 | 0.67 | 2\|0\|2 | 0 | 0.01 | 0 | 0.01 | 0 | −7.85 | 0.5 | 0.82 |
| D5-2 | 102724023 | | | | | | | | | | | | |
| P35579 | MYH9 | 18\|22\|26 | 66 | 22 | 23\|24\|21 | 0 | 0 | 0 | 0 | 0 | −56.97 | 0.97 | 0.92 |
| P31943 | HNRNPH1 | 38\|35\|38 | 111 | 37 | 44\|47\|54 | 0 | 0 | 0 | 0 | 0 | −124.15 | 0.77 | 0.92 |
| Q13356 | PPIL2 | 0\|0\|3 | 3 | 1 | 5\|4\|4 | 0 | 0 | 0 | 0 | 0 | −20.66 | 0.23 | 0.92 |
| Q8N1F7 | NUP93 | 3\|4\|4 | 11 | 3.67 | 4\|3\|6 | 0 | 0 | 0 | 0 | 0 | −13.81 | 0.85 | 0.92 |
| Q9BSV6 | TSEN34 | 3\|3\|3 | 9 | 3 | 3\|4\|0 | 0 | 0 | 0 | 0 | 0 | −6.46 | 1.29 | 0.83 |
| P38646 | HSPA9 | 28\|25\|34 | 87 | 29 | 36\|42\|34 | 0 | 0 | 0 | 0 | 0 | −100.12 | 0.78 | 0.92 |
| Q9UMS4 | PRPF19 | 5\|0\|0 | 5 | 1.67 | 8\|9\|12 | 0 | 0 | 0 | 0 | 0 | −44.25 | 0.17 | 0.92 |
| P60953 | CDC42 | 10\|7\|3 | 20 | 6.67 | 6\|6\|4 | 0 | 0 | 0 | 0 | 0 | −17.58 | 1.25 | 0.86 |
| Q9Y5B9 | SUPT16H | 7\|4\|8 | 19 | 6.33 | 12\|10\|9 | 0 | 0 | 0 | 0 | 0 | −35.41 | 0.61 | 0.92 |
| Q9GZT8-2 | NIF3L1 | 3\|3\|2 | 8 | 2.67 | 4\|4\|3 | 0 | 0 | 0 | 0 | 0 | −13.04 | 0.73 | 0.92 |
| O60610-2 | DIAPH1 | 5\|6\|6 | 17 | 5.67 | 6\|9\|8 | 0 | 0 | 0 | 0 | 0 | −22.9 | 0.74 | 0.92 |
| Q8N4C8-5 | MINK1 | 2\|3\|3 | 8 | 2.67 | 2\|3\|3 | 0 | 0 | 0 | 0 | 0 | −9.34 | 1 | 0.86 |
| Q9H0C8 | ILKAP | 0\|2\|3 | 5 | 1.67 | 2\|3\|3 | 0 | 0 | 0 | 0 | 0 | −13.44 | 0.63 | 0.87 |
| Q99460-2 | PSMD1 | 5\|4\|8 | 17 | 5.67 | 13\|9\|8 | 0 | 0 | 0 | 0 | 0 | −34.04 | 0.57 | 0.92 |
| Q9H6S0 | YTHDC2 | 2\|0\|0 | 2 | 0.67 | 2\|0\|2 | 0 | 0.01 | 0 | 0.01 | 0 | −7.85 | 0.5 | 0.82 |
| P60900-2 | PSMA6 | 2\|3\|0 | 5 | 1.67 | 4\|4\|2 | 0 | 0 | 0 | 0 | 0 | −16.29 | 0.5 | 0.92 |
| Q86UR5-12 | RIMS1 | 4\|0\|2 | 6 | 2 | 3\|3\|2 | 0 | 0 | 0 | 0 | 0 | −13.44 | 0.75 | 0.85 |
| P30566 | ADSL | 2\|2\|4 | 8 | 2.67 | 2\|2\|3 | 0 | 0.01 | 0 | 0.01 | 0 | −8.15 | 1.14 | 0.83 |
| Q8IX01-4 | SUGP2 | 2\|3\|2 | 7 | 2.33 | 24\|20\|24 | 0 | 0 | 0 | 0 | 0 | −93.96 | 0.1 | 0.92 |
| Q9NW13 | RBM28 | 0\|2\|3 | 5 | 1.67 | 5\|6\|7 | 0 | 0 | 0 | 0 | 0 | −27.96 | 0.28 | 0.92 |
| P38159 | RBMX | 11\|10\|14 | 35 | 11.67 | 34\|31\|32 | 0 | 0 | 0 | 0 | 0 | −111.88 | 0.36 | 0.92 |
| Q8IWZ3-6 | ANKHD1 | 6\|5\|10 | 21 | 7 | 6\|12\|10 | 0 | 0 | 0 | 0 | 0 | −29.32 | 0.75 | 0.91 |
| Q9Y285 | FARSA | 3\|0\|2 | 5 | 1.67 | 3\|5\|5 | 0 | 0 | 0 | 0 | 0 | −20.66 | 0.38 | 0.92 |
| P43034 | PAFAH1B1 | 7\|9\|6 | 22 | 7.33 | 7\|4\|8 | 0 | 0 | 0 | 0 | 0 | −16.44 | 1.16 | 0.89 |
| Q9BWM7 | SFXN3 | 3\|2\|3 | 8 | 2.67 | 3\|0\|4 | 0 | 0 | 0 | 0 | 0 | −7.98 | 1.14 | 0.84 |
| Q06124 | PTPN11 | 0\|4\|2 | 6 | 2 | 3\|4\|4 | 0 | 0 | 0 | 0 | 0 | −17.76 | 0.55 | 0.88 |
| Q9UHX1-4 | PUF60 | 7\|6\|7 | 20 | 6.67 | 6\|9\|6 | 0 | 0 | 0 | 0 | 0 | −18.77 | 0.95 | 0.92 |
| Q96HN2-2 | AHCYL2 | 5\|4\|6 | 15 | 5 | 7\|6\|5 | 0 | 0 | 0 | 0 | 0 | −18.31 | 0.83 | 0.92 |
| Q9UPQ9-1 | TNRC6B | 20\|20\|17 | 57 | 19 | 13\|15\|18 | 0 | 0 | 0 | 0 | 0 | −32.21 | 1.24 | 0.91 |
| P49916-4 | LIG3 | 0\|2\|0 | 2 | 0.67 | 2\|2\|0 | 0 | 0.01 | 0 | 0.01 | 0 | −7.85 | 0.5 | 0.82 |
| O95340 | PAPSS2 | 6\|6\|4 | 16 | 5.33 | 4\|7\|6 | 0 | 0 | 0 | 0 | 0 | −17.11 | 0.94 | 0.9 |
| O95347 | SMC2 | 11\|11\|10 | 32 | 10.67 | 14\|13\|18 | 0 | 0 | 0 | 0 | 0 | −41.87 | 0.71 | 0.92 |
| Q8IZP0-3 | ABI1 | 10\|13\|12 | 35 | 11.67 | 11\|11\|9 | 0 | 0 | 0 | 0 | 0 | −24.73 | 1.13 | 0.91 |
| Q99700-2 | ATXN2 | 0\|3\|5 | 8 | 2.67 | 4\|3\|3 | 0 | 0 | 0 | 0 | 0 | −16.29 | 0.8 | 0.86 |
| Q99729-3 | HNRNPAB | 4\|4\|4 | 12 | 4 | 4\|4\|5 | 0 | 0 | 0 | 0 | 0 | −12.29 | 0.92 | 0.92 |
| Q7L2H7-2 | EIF3M | 0\|0\|2 | 2 | 0.67 | 0\|2\|2 | 0 | 0.01 | 0 | 0.01 | 0 | −7.85 | 0.5 | 0.82 |
| Q9Y310 | RTCB | 11\|16\|18 | 45 | 15 | 15\|13\|13 | 0 | 0 | 0 | 0 | 0 | −35.19 | 1.1 | 0.91 |
| O75694-2 | NUP155 | 2\|2\|0 | 4 | 1.33 | 4\|3\|4 | 0 | 0 | 0 | 0 | 0 | −17.76 | 0.36 | 0.92 |
| P62829 | RPL23 | 10\|12\|12 | 34 | 11.33 | 14\|13\|14 | 0 | 0 | 0 | 0 | 0 | −36.85 | 0.83 | 0.92 |
| P62820 | RAB1A | 16\|14\|13 | 43 | 14.33 | 15\|18\|16 | 0 | 0 | 0 | 0 | 0 | −41.76 | 0.88 | 0.92 |
| P06733 | ENO1 | 87\|83\|87 | 257 | 85.67 | 85\|94\|105 | 0 | 0 | 0 | 0 | 0 | −216.77 | 0.9 | 0.92 |
| Q9Y4W6 | AFG3L2 | 3\|0\|3 | 6 | 2 | 3\|3\|6 | 0 | 0 | 0 | 0 | 0 | −19.16 | 0.5 | 0.92 |
| Q08378-2 | GOLGA3 | 0\|0\|2 | 2 | 0.67 | 6\|4\|0 | 0 | 0 | 0 | 0 | 0 | −15.92 | 0.2 | 0.92 |
| Q9NXH9-2 | TRMT1 | 9\|5\|9 | 23 | 7.67 | 8\|10\|10 | 0 | 0 | 0 | 0 | 0 | −29.32 | 0.82 | 0.92 |
| O95299 | NDUFA10 | 2\|2\|0 | 4 | 1.33 | 3\|3\|3 | 0 | 0 | 0 | 0 | 0 | −14.8 | 0.44 | 0.92 |
| Q92615 | LARP4B | 7\|8\|5 | 20 | 6.67 | 6\|7\|6 | 0 | 0 | 0 | 0 | 0 | −17.93 | 1.05 | 0.9 |
| Q99661 | KIF2C | 6\|4\|6 | 16 | 5.33 | 6\|6\|5 | 0 | 0 | 0 | 0 | 0 | −17.11 | 0.94 | 0.9 |
| P43490 | NAMPT | 16\|15\|16 | 47 | 15.67 | 18\|17\|19 | 0 | 0 | 0 | 0 | 0 | −44.65 | 0.87 | 0.92 |
| P10768 | ESD | 6\|5\|7 | 18 | 6 | 8\|9\|9 | 0 | 0 | 0 | 0 | 0 | −26.73 | 0.69 | 0.92 |
| Q9BZK7 | TBL1XR1 | 2\|2\|0 | 4 | 1.33 | 4\|2\|5 | 0 | 0 | 0 | 0 | 0 | −17.76 | 0.36 | 0.92 |
| P46063 | RECQL | 2\|3\|4 | 9 | 3 | 4\|4\|2 | 0 | 0 | 0 | 0 | 0 | −11.76 | 0.9 | 0.88 |
| O75964 | ATP5L | 0\|0\|2 | 2 | 0.67 | 2\|3\|0 | 0 | 0 | 0 | 0 | 0 | −9.18 | 0.4 | 0.84 |
| P42766 | RPL35 | 4\|0\|3 | 7 | 2.33 | 5\|3\|3 | 0 | 0 | 0 | 0 | 0 | −17.76 | 0.64 | 0.88 |
| P22087 | FBL | 3\|3\|3 | 9 | 3 | 6\|5\|6 | 0 | 0 | 0 | 0 | 0 | −18.88 | 0.53 | 0.92 |
| Q9UHD1 | CHORDC1 | 10\|15\|13 | 38 | 12.67 | 14\|14\|9 | 0 | 0 | 0 | 0 | 0 | −31.9 | 1.03 | 0.91 |
| Q96GX5-2 | MASTL | 3\|0\|0 | 3 | 1 | 0\|3\|5 | 0 | 0 | 0 | 0 | 0 | −13.21 | 0.38 | 0.86 |
| P53618 | COPB1 | 10\|10\|12 | 32 | 10.67 | 12\|13\|15 | 0 | 0 | 0 | 0 | 0 | −35.6 | 0.8 | 0.92 |
| P62318-2 | SNRPD3 | 2\|2\|0 | 4 | 1.33 | 2\|3\|2 | 0 | 0 | 0 | 0 | 0 | −11.98 | 0.57 | 0.92 |
| Q96JM3 | CHAMP1 | 2\|2\|2 | 6 | 2 | 6\|6\|5 | 0 | 0 | 0 | 0 | 0 | −20.94 | 0.35 | 0.92 |
| P50991 | CCT4 | 26\|20\|21 | 67 | 22.33 | 25\|24\|31 | 0 | 0 | 0 | 0 | 0 | −68.51 | 0.84 | 0.92 |
| P24666 | ACP1 | 4\|4\|5 | 13 | 4.33 | 3\|4\|4 | 0 | 0 | 0 | 0 | 0 | −9.93 | 1.18 | 0.87 |
| Q9GZS3 | WDR61 | 2\|3\|3 | 8 | 2.67 | 2\|4\|3 | 0 | 0 | 0 | 0 | 0 | −10.47 | 0.89 | 0.92 |
| Q96HC4 | PDLIM5 | 12\|7\|14 | 33 | 11 | 7\|7\|5 | 0 | 0 | 0 | 0 | 0 | −14.98 | 1.74 | 0.83 |
| Q9Y320-2 | TMX2 | 2\|0\|0 | 2 | 0.67 | 0\|2\|2 | 0 | 0.01 | 0 | 0.01 | 0 | −7.85 | 0.5 | 0.82 |
| P49327 | FASN | 66\|65\|65 | 196 | 65.33 | 65\|73\|73 | 0 | 0 | 0 | 0 | 0 | −156.56 | 0.93 | 0.92 |
| P00387-2 | CYB5R3 | 4\|3\|4 | 11 | 3.67 | 5\|5\|4 | 0 | 0 | 0 | 0 | 0 | −15.06 | 0.79 | 0.92 |
| O75340 | PDCD6 | 2\|0\|2 | 4 | 1.33 | 2\|2\|2 | 0 | 0 | 0 | 0 | 0 | −10.49 | 0.67 | 0.92 |
| P14618 | PKM | 80\|68\|71 | 219 | 73 | 80\|69\|81 | 0 | 0 | 0 | 0 | 0 | −174.79 | 0.95 | 0.92 |
| Q8TB61-3 | SLC35B2 | 0\|2\|0 | 2 | 0.67 | 0\|2\|2 | 0 | 0.01 | 0 | 0.01 | 0 | −7.85 | 0.5 | 0.82 |
| P25685 | DNAJB1 | 7\|8\|8 | 23 | 7.67 | 8\|11\|5 | 0 | 0 | 0 | 0 | 0 | −20.9 | 0.96 | 0.92 |
| P42167 | TMPO | 3\|2\|4 | 9 | 3 | 7\|6\|8 | 0 | 0 | 0 | 0 | 0 | −26.38 | 0.43 | 0.92 |
| P42166 | TMPO | 2\|2\|3 | 7 | 2.33 | 6\|5\|7 | 0 | 0 | 0 | 0 | 0 | −22.26 | 0.39 | 0.92 |
| P62195-2 | PSMC5 | 14\|14\|14 | 42 | 14 | 17\|15\|14 | 0 | 0 | 0 | 0 | 0 | −36.57 | 0.91 | 0.92 |
| Q8NFH3 | NUP43 | 4\|0\|3 | 7 | 2.33 | 5\|3\|3 | 0 | 0 | 0 | 0 | 0 | −17.76 | 0.64 | 0.88 |
| P18583-5 | SON | 6\|2\|6 | 14 | 4.67 | 12\|14\|8 | 0 | 0 | 0 | 0 | 0 | −44.69 | 0.41 | 0.92 |
| Q9NYK5 | MRPL39 | 4\|5\|4 | 13 | 4.33 | 4\|5\|6 | 0 | 0 | 0 | 0 | 0 | −14.62 | 0.87 | 0.92 |
| Q96PK6 | RBM14 | 21\|23\|21 | 65 | 21.67 | 23\|24\|23 | 0 | 0 | 0 | 0 | 0 | −54.65 | 0.93 | 0.92 |
| Q8N1G2 | CMTR1 | 2\|2\|4 | 8 | 2.67 | 5\|4\|5 | 0 | 0 | 0 | 0 | 0 | −16.93 | 0.57 | 0.92 |
| P41091 | EIF2S3 | 14\|19\|18 | 51 | 17 | 17\|22\|18 | 0 | 0 | 0 | 0 | 0 | −50 | 0.89 | 0.92 |
| O60231 | DHX16 | 2\|2\|3 | 7 | 2.33 | 5\|4\|4 | 0 | 0 | 0 | 0 | 0 | −15.6 | 0.54 | 0.92 |
| Q12906-7 | ILF3 | 21\|14\|19 | 54 | 18 | 29\|36\|30 | 0 | 0 | 0 | 0 | 0 | −99.68 | 0.57 | 0.92 |
| Q9BQE3 | TUBA1C | 81\|64\|72 | 217 | 72.33 | 71\|68\|73 | 0 | 0 | 0 | 0 | 0 | −159.29 | 1.02 | 0.92 |
| Q96KK5 | HIST1H2AH | 4\|5\|5 | 14 | 4.67 | 11\|9\|9 | 0 | 0 | 0 | 0 | 0 | −32.71 | 0.48 | 0.92 |
| Q9BQ39 | DDX50 | 0\|0\|2 | 2 | 0.67 | 4\|8\|5 | 0 | 0 | 0 | 0 | 0 | −26.53 | 0.12 | 0.92 |
| Q9NYY8 | FASTKD2 | 3\|4\|3 | 10 | 3.33 | 5\|5\|3 | 0 | 0 | 0 | 0 | 0 | −13.81 | 0.77 | 0.92 |
| P68366-2 | TUBA4A | 72\|62\|67 | 201 | 67 | 66\|67\|69 | 0 | 0 | 0 | 0 | 0 | −150.35 | 1 | 0.92 |
| Q9NS69 | TOMM22 | 2\|3\|0 | 5 | 1.67 | 3\|2\|7 | 0 | 0 | 0 | 0 | 0 | −18.99 | 0.42 | 0.92 |
| Q16658 | FSCN1 | 14\|15\|13 | 42 | 14 | 12\|10\|21 | 0 | 0 | 0 | 0 | 0 | −34.17 | 0.98 | 0.91 |
| Q9ULD2-2 | MTUS1 | 0\|0\|2 | 2 | 0.67 | 4\|4\|3 | 0 | 0 | 0 | 0 | 0 | −17.76 | 0.18 | 0.92 |
| Q9NXC5 | MIOS | 0\|0\|2 | 2 | 0.67 | 2\|2\|0 | 0 | 0.01 | 0 | 0.01 | 0 | −7.85 | 0.5 | 0.82 |
| P13489 | RNH1 | 6\|7\|10 | 23 | 7.67 | 7\|8\|9 | 0 | 0 | 0 | 0 | 0 | −22.42 | 0.96 | 0.9 |
| Q14839 | CHD4 | 0\|2\|5 | 7 | 2.33 | 13\|16\|14 | 0 | 0 | 0 | 0 | 0 | −65.05 | 0.16 | 0.92 |
| Q96124 | FUBP3 | 5\|4\|4 | 13 | 4.33 | 5\|6\|5 | 0 | 0 | 0 | 0 | 0 | −15.87 | 0.81 | 0.92 |
| P26639 | TARS | 24\|25\|31 | 80 | 26.67 | 37\|37\|34 | 0 | 0 | 0 | 0 | 0 | −96.82 | 0.74 | 0.92 |
| P50402 | EMD | 7\|7\|6 | 20 | 6.67 | 7\|7\|7 | 0 | 0 | 0 | 0 | 0 | −18.77 | 0.95 | 0.92 |
| Q96GA3 | LTV1 | 7\|6\|8 | 21 | 7 | 10\|8\|11 | 0 | 0 | 0 | 0 | 0 | −28.73 | 0.72 | 0.92 |
| P62899 | RPL31 | 4\|4\|3 | 11 | 3.67 | 3\|5\|3 | 0 | 0 | 0 | 0 | 0 | −11.41 | 1 | 0.88 |
| P31150 | GDI1 | 4\|6\|6 | 16 | 5.33 | 7\|5\|5 | 0 | 0 | 0 | 0 | 0 | −17.11 | 0.94 | 0.9 |
| Q86TG7 | PEG10 | 6\|5\|4 | 15 | 5 | 5\|5\|2 | 0 | 0 | 0 | 0 | 0 | −11.11 | 1.25 | 0.87 |
| P48444 | ARCN1 | 13\|14\|14 | 41 | 13.67 | 16\|16\|15 | 0 | 0 | 0 | 0 | 0 | −39.32 | 0.87 | 0.92 |
| Q9Y4E1-3 | WASHC2C | 2\|3\|2 | 7 | 2.33 | 2\|2\|4 | 0 | 0 | 0 | 0 | 0 | −9.34 | 0.88 | 0.87 |
| Q15233 | NONO | 45\|45\|49 | 139 | 46.33 | 48\|45\|47 | 0 | 0 | 0 | 0 | 0 | −102.03 | 0.99 | 0.92 |
| P29218 | IMPA1 | 3\|3\|5 | 11 | 3.67 | 5\|4\|6 | 0 | 0 | 0 | 0 | 0 | −16.28 | 0.73 | 0.92 |
| P35606-2 | COPB2 | 16\|15\|22 | 53 | 17.67 | 14\|22\|14 | 0 | 0 | 0 | 0 | 0 | −39.82 | 1.06 | 0.91 |
| O94776 | MTA2 | 8\|6\|13 | 27 | 9 | 16\|15\|16 | 0 | 0 | 0 | 0 | 0 | −52.61 | 0.57 | 0.92 |
| Q5JTZ9 | AARS2 | 3\|0\|3 | 6 | 2 | 2\|2\|2 | 0 | 0 | 0 | 0 | 0 | −10.49 | 1 | 0.83 |
| Q9UQ35 | SRRM2 | 7\|3\|8 | 18 | 6 | 18\|13\|15 | 0 | 0 | 0 | 0 | 0 | −58.89 | 0.39 | 0.92 |
| O00458 | IFRD1 | 4\|3\|4 | 11 | 3.67 | 41414 | 0 | 0 | 0 | 0 | 0 | −12.55 | 0.92 | 0.92 |
| Q9P2R7-2 | SUCLA2 | 5\|5\|6 | 16 | 5.33 | 10\|7\|6 | 0 | 0 | 0 | 0 | 0 | −22.9 | 0.7 | 0.92 |
| P16615 | ATP2A2 | 16\|13\|13 | 42 | 14 | 14\|15\|21 | 0 | 0 | 0 | 0 | 0 | −42.99 | 0.84 | 0.92 |
| Q9BRS2 | RIOK1 | 0\|0\|2 | 2 | 0.67 | 4\|4\|2 | 0 | 0 | 0 | 0 | 0 | −16.29 | 0.2 | 0.92 |
| Q9P0J0 | NDUFA13 | 0\|2\|0 | 2 | 0.67 | 0\|2\|2 | 0 | 0.01 | 0 | 0.01 | 0 | −7.85 | 0.5 | 0.82 |
| Q12972 | PPP1R8 | 2\|4\|4 | 10 | 3.33 | 5\|3\|5 | 0 | 0 | 0 | 0 | 0 | −15.6 | 0.77 | 0.92 |
| Q9P0J7 | KCMF1 | 4\|4\|5 | 13 | 4.33 | 0\|4\|5 | 0 | 0 | 0 | 0 | 0 | −7.25 | 1.44 | 0.84 |
| P29144 | TPP2 | 6\|0\|5 | 11 | 3.67 | 6\|9\|12 | 0 | 0 | 0 | 0 | 0 | −41.27 | 0.41 | 0.92 |
| P19525 | EIF2AK2 | 4\|3\|6 | 13 | 4.33 | 6\|9\|9 | 0 | 0 | 0 | 0 | 0 | −28.15 | 0.54 | 0.92 |
| Q9UKK9 | NUDT5 | 7\|6\|5 | 18 | 6 | 6\|7\|6 | 0 | 0 | 0 | 0 | 0 | −17.93 | 0.95 | 0.9 |
| Q8IVT2 | MISP | 19\|19\|19 | 57 | 19 | 24\|26\|24 | 0 | 0 | 0 | 0 | 0 | −62.73 | 0.77 | 0.92 |
| P53992 | SEC24C | 9\|8\|8 | 25 | 8.33 | 5\|6\|9 | 0 | 0 | 0 | 0 | 0 | −14.71 | 1.25 | 0.89 |
| Q00839-2 | HNRNPU | 26\|23\|32 | 81 | 27 | 33\|31\|29 | 0 | 0 | 0 | 0 | 0 | −79.63 | 0.87 | 0.92 |
| P14324-2 | FDPS | 4\|4\|4 | 12 | 4 | 6\|5\|5 | 0 | 0 | 0 | 0 | 0 | −15.87 | 0.75 | 0.92 |
| Q00610-2 | CLTC | 27\|23\|28 | 78 | 26 | 28\|26\|29 | 0 | 0 | 0 | 0 | 0 | −67.26 | 0.94 | 0.92 |
| O15355 | PPM1G | 3\|0\|0 | 3 | 1 | 3\|2\|2 | 0 | 0 | 0 | 0 | 0 | −11.98 | 0.43 | 0.85 |
| Q99832 | CCT7 | 20\|24\|21 | 65 | 21.67 | 24\|29\|28 | 0 | 0 | 0 | 0 | 0 | −69.75 | 0.8 | 0.92 |
| P04150-2 | NR3C1 | 2\|2\|4 | 8 | 2.67 | 2\|2\|3 | 0 | 0.01 | 0 | 0.01 | 0 | −8.15 | 1.14 | 0.83 |
| P01111 | NRAS | 9\|3\|6 | 18 | 6 | 4\|3\|6 | 0 | 0 | 0 | 0 | 0 | −13.81 | 1.38 | 0.83 |
| Q9H2P0 | ADNP | 10\|12\|10 | 32 | 10.67 | 19\|18\|15 | 0 | 0 | 0 | 0 | 0 | −50.87 | 0.62 | 0.92 |
| Q9BTE6 | AARSD1 | 3\|4\|6 | 13 | 4.33 | 2\|3\|4 | 0 | 0.01 | 0 | 0.01 | 0 | −9.04 | 1.44 | 0.82 |
| P61978-3 | HNRNPK | 24\|33\|34 | 91 | 30.33 | 35\|43\|39 | 0 | 0 | 0 | 0 | 0 | −108.39 | 0.78 | 0.92 |
| Q13111 | CHAF1A | 11\|7\|11 | 29 | 9.67 | 9\|8\|11 | 0 | 0 | 0 | 0 | 0 | −25.73 | 1.04 | 0.91 |
| P35527 | KRT9 | 69\|47\|11 | 127 | 42.33 | 50\|39\|35 | 0 | 0 | 0 | 0 | 0 | −146.99 | 1.02 | 0.92 |
| P61978 | HNRNPK | 26\|33\|35 | 94 | 31.33 | 35\|44\|39 | 0 | 0 | 0 | 0 | 0 | −105.96 | 0.8 | 0.92 |
| Q9P265 | DIP2B | 2\|0\|2 | 4 | 1.33 | 2\|4\|0 | 0 | 0 | 0 | 0 | 0 | −10.24 | 0.67 | 0.92 |
| Q14247 | CTTN | 15\|13\|17 | 45 | 15 | 14\|11\|14 | 0 | 0 | 0 | 0 | 0 | −29.78 | 1.15 | 0.91 |
| Q14244 | MAP7 | 35\|33\|43 | 111 | 37 | 31\|27\|37 | 0 | 0 | 0 | 0 | 0 | −66.56 | 1.17 | 0.92 |
| Q14240 | EIF4A2 | 22\|21\|24 | 67 | 22.33 | 21\|29\|27 | 0 | 0 | 0 | 0 | 0 | −63.14 | 0.87 | 0.92 |
| Q8IWX8 | CHERP | 0\|2\|0 | 2 | 0.67 | 6\|15\|7 | 0 | 0 | 0 | 0 | 0 | −42.4 | 0.07 | 0.92 |
| P24928 | POLR2A | 4\|4\|4 | 12 | 4 | 5\|5\|7 | 0 | 0 | 0 | 0 | 0 | −17.11 | 0.71 | 0.92 |
| Q9BRJ2 | MRPL45 | 4\|5\|4 | 13 | 4.33 | 4\|2\|4 | 0 | 0 | 0 | 0 | 0 | −8.76 | 1.3 | 0.86 |
| Q5JSZ5 | PRRC2B | 11\|8\|10 | 29 | 9.67 | 10\|7\|9 | 0 | 0 | 0 | 0 | 0 | −21.76 | 1.12 | 0.9 |
| Q13200 | PSMD2 | 10\|13\|15 | 38 | 12.67 | 13\|16\|17 | 0 | 0 | 0 | 0 | 0 | −43.15 | 0.83 | 0.92 |
| P63000 | RAC1 | 7\|7\|8 | 22 | 7.33 | 9\|8\|6 | 0 | 0 | 0 | 0 | 0 | −19.69 | 0.96 | 0.9 |
| Q8N163 | CCAR2 | 24\|21\|19 | 64 | 21.33 | 19\|25\|26 | 0 | 0 | 0 | 0 | 0 | −57.79 | 0.91 | 0.92 |
| Tx0002 | Tx0002 | 645\|609\|601 | 1855 | 618.33 | 642\|671\| | 0 | 0 | 0 | 0 | 0 | #NAME? | 0.93 | 0.92 |
| | | | | | 688 | | | | | | | | |
| Q15758 | SLC1A5 | 4\|6\|3 | 13 | 4.33 | 4\|4\|5 | 0 | 0 | 0 | 0 | 0 | −13.81 | 1 | 0.88 |
| Q8WX93-3 | PALLD | 12\|7\|18 | 37 | 12.33 | 8\|7\|8 | 0 | 0.01 | 0 | 0.01 | 0 | −19.69 | 1.61 | 0.81 |
| O14617-3 | AP3D1 | 3\|0\|2 | 5 | 1.67 | 2\|2\|5 | 0 | 0 | 0 | 0 | 0 | −14.8 | 0.56 | 0.92 |
| Q9GZR7 | DDX24 | 0\|3\|0 | 3 | 1 | 3\|2\|4 | 0 | 0 | 0 | 0 | 0 | −14.8 | 0.33 | 0.92 |
| Q9H4A3-2 | WNK1 | 3\|3\|7 | 13 | 4.33 | 7\|8\|8 | 0 | 0 | 0 | 0 | 0 | −26.82 | 0.57 | 0.92 |
| O15427 | SLC16A3 | 5\|3\|5 | 13 | 4.33 | 3\|2\|4 | 0 | 0 | 0 | 0 | 0 | −9.04 | 1.44 | 0.83 |
| Q9NWB6 | ARGLU1 | 2\|2\|2 | 6 | 2 | 4\|3\|3 | 0 | 0 | 0 | 0 | 0 | −11.76 | 0.6 | 0.92 |
| Q16795 | NDUFA9 | 3\|2\|3 | 8 | 2.67 | 5\|5\|4 | 0 | 0 | 0 | 0 | 0 | −16.93 | 0.57 | 0.92 |
| P62280 | RPS11 | 13\|10\|17 | 40 | 13.33 | 17\|11\|13 | 0 | 0 | 0 | 0 | 0 | −36.85 | 0.98 | 0.91 |
| P45880-2 | VDAC2 | 4\|5\|4 | 13 | 4.33 | 4\|3\|5 | 0 | 0 | 0 | 0 | 0 | −11.11 | 1.08 | 0.88 |
| P25786 | PSMA1 | 3\|2\|2 | 7 | 2.33 | 3\|3\|0 | 0 | 0 | 0 | 0 | 0 | −6.87 | 1.17 | 0.83 |
| Q9NYJ8 | TAB2 | 3\|2\|0 | 5 | 1.67 | 3\|0\|3 | 0 | 0 | 0 | 0 | 0 | −10.39 | 0.83 | 0.83 |
| Q96RP9 | GFM1 | 8\|4\|6 | 18 | 6 | 8\|7\|9 | 0 | 0 | 0 | 0 | 0 | −26.03 | 0.75 | 0.92 |
| O60488-2 | ACSL4 | 2\|3\|4 | 9 | 3 | 3\|4\|2 | 0 | 0 | 0 | 0 | 0 | −10.47 | 1 | 0.86 |
| P61158 | ACTR3 | 9\|9\|11 | 29 | 9.67 | 11\|7\|9 | 0 | 0 | 0 | 0 | 0 | −21.48 | 1.07 | 0.91 |
| P56134-4 | ATP5J2 | 4\|5\|5 | 14 | 4.67 | 5\|4\|3 | 0 | 0 | 0 | 0 | 0 | −11.11 | 1.17 | 0.88 |
| O95602 | POLR1A | 3\|2\|5 | 10 | 3.33 | 7\|7\|7 | 0 | 0 | 0 | 0 | 0 | −26.38 | 0.48 | 0.92 |
| O00178 | GTPBP1 | 2\|0\|0 | 2 | 0.67 | 2\|3\|0 | 0 | 0 | 0 | 0 | 0 | −9.18 | 0.4 | 0.84 |
| Q15054-3 | POLD3 | 3\|2\|2 | 7 | 2.33 | 2\|2\|3 | 0 | 0 | 0 | 0 | 0 | −8.15 | 1 | 0.85 |
| P62191 | PSMC1 | 8\|12\|10 | 30 | 10 | 11\|12\|11 | 0 | 0 | 0 | 0 | 0 | −31.51 | 0.88 | 0.91 |
| P11413 | G6PD | 13\|12\|13 | 38 | 12.67 | 11\|12\|11 | 0 | 0 | 0 | 0 | 0 | −25.35 | 1.12 | 0.91 |
| P12036-2 | NEFH | 3\|2\|2 | 7 | 2.33 | 3\|2\|2 | 0 | 0 | 0 | 0 | 0 | −8.15 | 1 | 0.85 |
| P11142 | HSPA8 | 45\|48\|56 | 149 | 49.67 | 62\|51\|54 | 0 | 0 | 0 | 0 | 0 | −134.65 | 0.89 | 0.92 |
| Q04864-2 | REL | 0\|2\|0 | 2 | 0.67 | 2\|3\|0 | 0 | 0 | 0 | 0 | 0 | −9.18 | 0.4 | 0.84 |
| Q96EK5 | KIF1BP | 2\|2\|3 | 7 | 2.33 | 2\|2\|2 | 0 | 0 | 0 | 0 | 0 | −6.96 | 1.17 | 0.84 |
| Q9BVP2-2 | GNL3 | 4\|2\|3 | 9 | 3 | 6\|7\|5 | 0 | 0 | 0 | 0 | 0 | −22.26 | 0.5 | 0.92 |
| Q13501-2 | SQSTM1 | 11\|9\|8 | 28 | 9.33 | 9\|7\|7 | 0 | 0 | 0 | 0 | 0 | −18.23 | 1.22 | 0.9 |
| P61081 | UBE2M | 8\|5\|9 | 22 | 7.33 | 8\|6\|6 | 0 | 0 | 0 | 0 | 0 | −19.16 | 1.1 | 0.9 |
| P62266 | RPS23 | 2\|2\|2 | 6 | 2 | 4\|3\|3 | 0 | 0 | 0 | 0 | 0 | −11.76 | 0.6 | 0.92 |
| P62263 | RPS14 | 21\|16\|19 | 56 | 18.67 | 20\|16\|18 | 0 | 0 | 0 | 0 | 0 | −43.09 | 1.04 | 0.91 |
| P62269 | RPS18 | 6\|4\|8 | 18 | 6 | 4\|3\|6 | 0 | 0 | 0 | 0 | 0 | −12.29 | 1.38 | 0.85 |
| Q9BR76 | CORO1B | 2\|2\|0 | 4 | 1.33 | 3\|3\|2 | 0 | 0 | 0 | 0 | 0 | −13.44 | 0.5 | 0.92 |
| Q9H2M9 | RAB3GAP2 | 0\|0\|2 | 2 | 0.67 | 2\|0\|2 | 0 | 0.01 | 0 | 0.01 | 0 | −7.85 | 0.5 | 0.82 |
| Q9H0U3 | MAGT1 | 0\|0\|2 | 2 | 0.67 | 2\|0\|2 | 0 | 0.01 | 0 | 0.01 | 0 | −7.85 | 0.5 | 0.82 |
| Q08945 | SSRP1 | 7\|5\|5 | 17 | 5.67 | 9\|5\|4 | 0 | 0 | 0 | 0 | 0 | −16.73 | 0.94 | 0.9 |
| Q9H0A0 | NAT10 | 7\|3\|10 | 20 | 6.67 | 15\|19\|18 | 0 | 0 | 0 | 0 | 0 | −67.47 | 0.38 | 0.92 |
| Q96C36 | PYCR2 | 2\|2\|3 | 7 | 2.33 | 3\|2\|3 | 0 | 0 | 0 | 0 | 0 | −9.34 | 0.88 | 0.87 |
| P07195 | LDHB | 12\|15\|11 | 38 | 12.67 | 11\|12\|12 | 0 | 0 | 0 | 0 | 0 | −27.98 | 1.09 | 0.91 |
| P11498 | PC | 809\|760\|796 | 2365 | 788.33 | 756\|751\| | 0 | 0 | 0 | 0 | 0 | #NAME? | 1.04 | 0.92 |
| | | | | | 768 | | | | | | | | |
| O00330 | PDHX | 4\|2\|4 | 10 | 3.33 | 4\|0\|3 | 0 | 0.01 | 0 | 0.01 | 0 | −7.98 | 1.43 | 0.81 |
| P23396 | RPS3 | 34\|31\|31 | 96 | 32 | 32\|35\|35 | 0 | 0 | 0 | 0 | 0 | −77.76 | 0.94 | 0.92 |
| O95163 | IKBKAP | 3\|3\|4 | 10 | 3.33 | 3\|4\|3 | 0 | 0 | 0 | 0 | 0 | −10.22 | 1 | 0.88 |
| Q96EY5 | MVB12A | 2\|2\|2 | 6 | 2 | 0\|2\|3 | 0 | 0 | 0 | 0 | 0 | −5.71 | 1.2 | 0.81 |
| Q9NXV6 | CDKN2AIP | 4\|4\|9 | 17 | 5.67 | 2\|7\|6 | 0 | 0 | 0 | 0 | 0 | −14.56 | 1.13 | 0.87 |
| P13639 | EEF2 | 69\|52\|79 | 200 | 66.67 | 85\|75\|83 | 0 | 0 | 0 | 0 | 0 | −218.29 | 0.82 | 0.92 |
| P52907 | CAPZA1 | 7\|8\|7 | 22 | 7.33 | 6\|6\|4 | 0 | 0 | 0 | 0 | 0 | −11.48 | 1.38 | 0.88 |
| P62841 | RPS15 | 5\|6\|9 | 20 | 6.67 | 5\|6\|7 | 0 | 0 | 0 | 0 | 0 | −16.7 | 1.11 | 0.89 |
| P15880 | RPS2 | 12\|16\|15 | 43 | 14.33 | 11\|12\|12 | 0 | 0 | 0 | 0 | 0 | −26.53 | 1.23 | 0.91 |
| Q9BXS5 | AP1M1 | 3\|5\|5 | 13 | 4.33 | 6\|8\|8 | 0 | 0 | 0 | 0 | 0 | −25.47 | 0.59 | 0.92 |
| O75330-2 | HMMR | 2\|2\|0 | 4 | 1.33 | 2\|3\|0 | 0 | 0 | 0 | 0 | 0 | −9.18 | 0.8 | 0.83 |
| P55809 | OXCT1 | 12\|11\|15 | 38 | 12.67 | 13\|18\|17 | 0 | 0 | 0 | 0 | 0 | −43.89 | 0.79 | 0.92 |
| Q9Y6Y8 | SEC23IP | 2\|0\|0 | 2 | 0.67 | 3\|2\|2 | 0 | 0 | 0 | 0 | 0 | −11.98 | 0.29 | 0.92 |
| O15160 | POLR1C | 6\|4\|3 | 13 | 4.33 | 4\|5\|6 | 0 | 0 | 0 | 0 | 0 | −16.28 | 0.87 | 0.89 |
| Q99829 | CPNE1 | 5\|6\|4 | 15 | 5 | 9\|9\|10 | 0 | 0 | 0 | 0 | 0 | −31.36 | 0.54 | 0.92 |
| P08243-2 | ASNS | 10\|11\|10 | 31 | 10.33 | 13\|12\|19 | 0 | 0 | 0 | 0 | 0 | −40.61 | 0.7 | 0.92 |
| P78527 | PRKDC | 16\|19\|13 | 48 | 16 | 16\|20\|26 | 0 | 0 | 0 | 0 | 0 | −58.13 | 0.77 | 0.92 |
| Q92747 | ARPC1A | 3\|2\|4 | 9 | 3 | 3\|2\|3 | 0 | 0 | 0 | 0 | 0 | −9.34 | 1.12 | 0.85 |
| O00165-5 | HAX1 | 2\|2\|2 | 6 | 2 | 3\|3\|3 | 0 | 0 | 0 | 0 | 0 | −10.47 | 0.67 | 0.92 |
| O43447-2 | PPIH | 2\|0\|0 | 2 | 0.67 | 2\|2\|0 | 0 | 0.01 | 0 | 0.01 | 0 | −7.85 | 0.5 | 0.82 |
| Q6ULP2-8 | AFTPH | 4\|2\|4 | 10 | 3.33 | 6\|7\|6 | 0 | 0 | 0 | 0 | 0 | −23.65 | 0.53 | 0.92 |
| Q99873-2 | PRMT1 | 5\|5\|6 | 16 | 5.33 | 9\|5\|3 | 0 | 0 | 0 | 0 | 0 | −15.43 | 0.94 | 0.9 |
| P31153 | MAT2A | 7\|10\|7 | 24 | 8 | 9\|10\|8 | 0 | 0 | 0 | 0 | 0 | −24.48 | 0.89 | 0.91 |
| Q6VY07 | PACS1 | 2\|0\|0 | 2 | 0.67 | 0\|2\|5 | 0 | 0 | 0 | 0 | 0 | −11.64 | 0.29 | 0.92 |
| P27448-8 | MARK3 | 0\|0\|2 | 2 | 0.67 | 5\|4\|3 | 0 | 0 | 0 | 0 | 0 | −19.16 | 0.17 | 0.92 |
| P57088 | TMEM33 | 5\|3\|3 | 11 | 3.67 | 4\|5\|2 | 0 | 0 | 0 | 0 | 0 | −11.41 | 1 | 0.87 |
| P61981 | YWHAG | 13\|9\|16 | 38 | 12.67 | 11\|11\|16 | 0 | 0 | 0 | 0 | 0 | −34.8 | 1 | 0.91 |
| Q14258 | TRIM25 | 5\|6\|7 | 18 | 6 | 8\|8\|9 | 0 | 0 | 0 | 0 | 0 | −25.43 | 0.72 | 0.92 |
| Q9UM54-6 | MYO6 | 9\|11\|9 | 29 | 9.67 | 28\|29\|28 | 0 | 0 | 0 | 0 | 0 | −97.72 | 0.34 | 0.92 |
| Q5VTR2 | RNF20 | 3\|6\|6 | 15 | 5 | 6\|5\|8 | 0 | 0 | 0 | 0 | 0 | −21.48 | 0.79 | 0.92 |
| Q9UJS0 | SLC25A13 | 16\|13\|10 | 39 | 13 | 12\|14\|16 | 0 | 0 | 0 | 0 | 0 | −38.08 | 0.93 | 0.91 |
| Q02543 | RPL18A | 7\|7\|10 | 24 | 8 | 10\|6\|9 | 0 | 0 | 0 | 0 | 0 | −22.07 | 0.96 | 0.9 |
| Q6IBS0 | TWF2 | 3\|2\|3 | 8 | 2.67 | 2\|2\|2 | 0 | 0 | 0 | 0 | 0 | −6.96 | 1.33 | 0.83 |
| Q9Y3Y2-4 | CHTOP | 12\|8\|9 | 29 | 9.67 | 9\|10\|8 | 0 | 0 | 0 | 0 | 0 | −22.92 | 1.07 | 0.9 |
| Q6P2E9 | EDC4 | 4\|4\|2 | 10 | 3.33 | 3\|4\|4 | 0 | 0 | 0 | 0 | 0 | −13.04 | 0.91 | 0.88 |
| P49189 | ALDH9A1 | 5\|6\|6 | 17 | 5.67 | 8\|6\|8 | 0 | 0 | 0 | 0 | 0 | −21.63 | 0.77 | 0.92 |
| P39656-3 | DDOST | 2\|0\|3 | 5 | 1.67 | 3\|2\|2 | 0 | 0 | 0 | 0 | 0 | −11.98 | 0.71 | 0.85 |
| Q9NUQ8 | ABCF3 | 3\|2\|4 | 9 | 3 | 3\|3\|4 | 0 | 0 | 0 | 0 | 0 | −11.76 | 0.9 | 0.88 |
| P33176 | KIF5B | 17\|19\|17 | 53 | 17.67 | 20\|17\|18 | 0 | 0 | 0 | 0 | 0 | −42.78 | 0.96 | 0.91 |
| P63104 | YWHAZ | 8\|5\|7 | 20 | 6.67 | 9\|7\|8 | 0 | 0 | 0 | 0 | 0 | −24.14 | 0.83 | 0.92 |
| Q13309 | SKP2 | 2\|2\|3 | 7 | 2.33 | 3\|4\|4 | 0 | 0 | 0 | 0 | 0 | −13.04 | 0.64 | 0.92 |
| Q9ULX3 | NOB1 | 4\|5\|8 | 17 | 5.67 | 4\|5\|4 | 0 | 0 | 0 | 0 | 0 | −12.29 | 1.31 | 0.85 |
| Q71UM5 | RPS27L | 6\|4\|7 | 17 | 5.67 | 9\|7\|5 | 0 | 0 | 0 | 0 | 0 | −22.13 | 0.81 | 0.92 |
| Q14677 | CLINT1 | 6\|4\|5 | 15 | 5 | 4\|5\|5 | 0 | 0 | 0 | 0 | 0 | −13.48 | 1.07 | 0.89 |
| Q96RQ3 | MCCC1 | 93\|91\|98 | 282 | 94 | 107\|113\| | 0 | 0 | 0 | 0 | 0 | −255.13 | 0.87 | 0.92 |
| | | | | | 106 | | | | | | | | |
| P61160 | ACTR2 | 0\|2\|0 | 2 | 0.67 | 5\|0\|0 | 0 | 0.01 | 0 | 0.01 | 0 | −8.81 | 0.4 | 0.83 |
| Q92499 | DDX1 | 9\|9\|8 | 26 | 8.67 | 6\|12\|10 | 0 | 0 | 0 | 0 | 0 | −24.14 | 0.93 | 0.92 |
| Q9H0B6-2 | KLC2 | 3\|3\|0 | 6 | 2 | 2\|3\|3 | 0 | 0 | 0 | 0 | 0 | −13.44 | 0.75 | 0.86 |
| Q7Z2W4 | ZC3HAV1 | 12\|14\|12 | 38 | 12.67 | 16\|13\|21 | 0 | 0 | 0 | 0 | 0 | −44.68 | 0.76 | 0.92 |
| P11177-2 | PDHB | 8\|9\|9 | 26 | 8.67 | 7\|12\|8 | 0 | 0 | 0 | 0 | 0 | −22.92 | 0.96 | 0.92 |
| P55039 | DRG2 | 3\|5\|5 | 13 | 4.33 | 4\|5\|5 | 0 | 0 | 0 | 0 | 0 | −15.06 | 0.93 | 0.89 |
| Q9NSD9 | FARSB | 10\|9\|11 | 30 | 10 | 11\|11\|9 | 0 | 0 | 0 | 0 | 0 | −26.21 | 0.97 | 0.91 |
| Q13490-2 | BIRC2 | 8\|6\|4 | 18 | 6 | 7\|7\|8 | 0 | 0 | 0 | 0 | 0 | −23.44 | 0.82 | 0.9 |
| Q9NZI8 | IGF2BP1 | 5\|7\|11 | 23 | 7.67 | 11\|8\|8 | 0 | 0 | 0 | 0 | 0 | −28 | 0.85 | 0.91 |
| P62070 | RRAS2 | 3\|0\|3 | 6 | 2 | 2\|2\|3 | 0 | 0 | 0 | 0 | 0 | −11.98 | 0.86 | 0.85 |
| Q13573 | SNW1 | 0\|0\|2 | 2 | 0.67 | 8\|9\|5 | 0 | 0 | 0 | 0 | 0 | −33.89 | 0.09 | 0.92 |
| Q92769-3 | HDAC2 | 5\|5\|6 | 16 | 5.33 | 5\|10\|6 | 0 | 0 | 0 | 0 | 0 | −20.36 | 0.76 | 0.92 |
| P62277 | RPS13 | 4\|3\|4 | 11 | 3.67 | 5\|5\|3 | 0 | 0 | 0 | 0 | 0 | −13.81 | 0.85 | 0.92 |
| Q9UJV9 | DDX41 | 5\|5\|5 | 15 | 5 | 6\|4\|5 | 0 | 0 | 0 | 0 | 0 | −13.18 | 1 | 0.92 |
| Q96EP5-2 | DAZAP1 | 0\|3\|0 | 3 | 1 | 2\|2\|3 | 0 | 0 | 0 | 0 | 0 | −11.98 | 0.43 | 0.85 |
| Q53GS9 | USP39 | 2\|4\|3 | 9 | 3 | 2\|5\|4 | 0 | 0 | 0 | 0 | 0 | −13.04 | 0.82 | 0.88 |
| P05023 | ATP1A1 | 6\|5\|11 | 22 | 7.33 | 6\|7\|7 | 0 | 0 | 0 | 0 | 0 | −19.16 | 1.1 | 0.89 |
| Q5T6F2 | UBAP2 | 0\|3\|4 | 7 | 2.33 | 5\|4\|3 | 0 | 0 | 0 | 0 | 0 | −19.16 | 0.58 | 0.92 |
| Q07065 | CKAP4 | 56\|56\|59 | 171 | 57 | 48\|49\|47 | 0 | 0 | 0 | 0 | 0 | −90.87 | 1.19 | 0.92 |
| P37108 | SRP14 | 5\|7\|6 | 18 | 6 | 4\|4\|3 | 0 | 0 | 0 | 0 | 0 | −8.5 | 1.64 | 0.83 |
| P22234 | PAICS | 14\|14\|15 | 43 | 14.33 | 15\|14\|12 | 0 | 0 | 0 | 0 | 0 | −30.67 | 1.05 | 0.91 |
| P50990 | CCT8 | 25\|22\|29 | 76 | 25.33 | 42\|50\|44 | 0 | 0 | 0 | 0 | 0 | −137.42 | 0.56 | 0.92 |
| Q16401-2 | PSMD5 | 2\|2\|2 | 6 | 2 | 3\|0\|3 | 0 | 0 | 0 | 0 | 0 | −6.87 | 1 | 0.92 |
| P31040 | SDHA | 14\|13\|16 | 43 | 14.33 | 20\|17\|19 | 0 | 0 | 0 | 0 | 0 | −50.49 | 0.77 | 0.92 |
| P51153 | RAB13 | 4\|3\|5 | 12 | 4 | 6\|5\|5 | 0 | 0 | 0 | 0 | 0 | −17.58 | 0.75 | 0.92 |
| P51151 | RAB9A | 4\|2\|3 | 9 | 3 | 3\|3\|3 | 0 | 0 | 0 | 0 | 0 | −10.47 | 1 | 0.86 |
| O00303 | EIF3F | 8\|11\|10 | 29 | 9.67 | 11\|12\|11 | 0 | 0 | 0 | 0 | 0 | −31.51 | 0.85 | 0.92 |
| P02786 | TFRC | 0\|0\|2 | 2 | 0.67 | 0\|3\|4 | 0 | 0 | 0 | 0 | 0 | −11.81 | 0.29 | 0.92 |
| P21796 | VDAC1 | 4\|3\|4 | 11 | 3.67 | 3\|2\|2 | 0 | 0.01 | 0 | 0.01 | 0 | −6.68 | 1.57 | 0.81 |
| O14579 | COPE | 6\|8\|8 | 22 | 7.33 | 8\|10\|7 | 0 | 0 | 0 | 0 | 0 | −23.68 | 0.88 | 0.92 |
| Q9UL25 | RAB21 | 4\|3\|5 | 12 | 4 | 5\|7\|4 | 0 | 0 | 0 | 0 | 0 | −17.58 | 0.75 | 0.92 |
| P0DMV9 | HSPA1B | 51\|48\|51 | 150 | 50 | 46\|41\|40 | 0 | 0 | 0 | 0 | 0 | −82.45 | 1.18 | 0.92 |
| P18621 | RPL17 | 8\|5\|9 | 22 | 7.33 | 5\|5\|8 | 0 | 0 | 0 | 0 | 0 | −16.7 | 1.22 | 0.89 |
| P62854 | RPS26 | 6\|4\|5 | 15 | 5 | 5\|5\|7 | 0 | 0 | 0 | 0 | 0 | −17.11 | 0.88 | 0.9 |
| P52597 | HNRNPF | 29\|22\|23 | 74 | 24.67 | 28\|31\|32 | 0 | 0 | 0 | 0 | 0 | −78.85 | 0.81 | 0.92 |
| P17655 | CAPN2 | 8\|4\|7 | 19 | 6.33 | 7\|10\|6 | 0 | 0 | 0 | 0 | 0 | −24.75 | 0.83 | 0.9 |
| P09211 | GSTP1 | 3\|3\|4 | 10 | 3.33 | 5\|5\|4 | 0 | 0 | 0 | 0 | 0 | −15.06 | 0.71 | 0.92 |
| Q13310-2 | PABPC4 | 2\|0\|0 | 2 | 0.67 | 2\|3\|4 | 0 | 0 | 0 | 0 | 0 | −14.8 | 0.22 | 0.92 |
| Q9BZX2 | UCK2 | 0\|2\|2 | 4 | 1.33 | 5\|4\|3 | 0 | 0 | 0 | 0 | 0 | −19.16 | 0.33 | 0.92 |
| A1X283 | SH3PXD2B | 2\|2\|4 | 8 | 2.67 | 7\|6\|6 | 0 | 0 | 0 | 0 | 0 | −23.65 | 0.42 | 0.92 |
| Q9H974-2 | QTRT2 | 2\|2\|0 | 4 | 1.33 | 2\|4\|4 | 0 | 0 | 0 | 0 | 0 | −16.29 | 0.4 | 0.92 |
| Q15020 | SART3 | 9\|6\|4 | 19 | 6.33 | 13\|4\|6 | 0 | 0 | 0 | 0 | 0 | −24.37 | 0.83 | 0.9 |
| Q9Y277 | VDAC3 | 6\|10\|7 | 23 | 7.67 | 8\|8\|8 | 0 | 0 | 0 | 0 | 0 | −22.42 | 0.96 | 0.9 |
| Q14914-2 | PTGR1 | 6\|5\|6 | 17 | 5.67 | 2\|3\|7 | 0 | 0 | 0 | 0 | 0 | −9.48 | 1.42 | 0.86 |
| Q8NI27 | THOC2 | 0\|0\|3 | 3 | 1 | 5\|3\|4 | 0 | 0 | 0 | 0 | 0 | −19.16 | 0.25 | 0.92 |
| P63092-3 | GNAS | 4\|4\|4 | 12 | 4 | 3\|4\|4 | 0 | 0 | 0 | 0 | 0 | −9.93 | 1.09 | 0.88 |
| Q9GZL7 | WDR12 | 4\|6\|6 | 16 | 5.33 | 4\|8\|7 | 0 | 0 | 0 | 0 | 0 | −19.6 | 0.84 | 0.92 |
| P30453 | HLA-A | 4\|2\|0 | 6 | 2 | 5\|3\|0 | 0 | 0 | 0 | 0 | 0 | −13.21 | 0.75 | 0.85 |
| P35610 | SOAT1 | 3\|3\|2 | 8 | 2.67 | 2\|2\|3 | 0 | 0 | 0 | 0 | 0 | −8.15 | 1.14 | 0.85 |
| P00403 | MT-CO2 | 2\|3\|3 | 8 | 2.67 | 4\|4\|2 | 0 | 0 | 0 | 0 | 0 | −11.76 | 0.8 | 0.92 |
| P17812 | CTPS1 | 12\|9\|12 | 33 | 11 | 11\|9\|10 | 0 | 0 | 0 | 0 | 0 | −25 | 1.1 | 0.91 |
| Q641Q2-2 | WASHC2A | 2\|3\|2 | 7 | 2.33 | 2\|3\|5 | 0 | 0 | 0 | 0 | 0 | −11.76 | 0.7 | 0.92 |
| P08670 | VIM | 60\|58\|65 | 183 | 61 | 74\|76\|70 | 0 | 0 | 0 | 0 | 0 | −178.64 | 0.83 | 0.92 |
| P32969 | RPL9P8; | 10\|8\|10 | 28 | 9.33 | 9\|13\|21 | 0 | 0 | 0 | 0 | 0 | −42.67 | 0.65 | 0.92 |
| | RPL9; | | | | | | | | | | | | |
| | RPL9P7; | | | | | | | | | | | | |
| | RPL9 | | | | | | | | | | | | |
| Q13177 | PAK2 | 6\|7\|10 | 23 | 7.67 | 6\|7\|7 | 0 | 0 | 0 | 0 | 0 | −17.62 | 1.15 | 0.89 |
| P42224 | STAT1 | 3\|8\|8 | 19 | 6.33 | 14\|8\|9 | 0 | 0 | 0 | 0 | 0 | −37.77 | 0.61 | 0.92 |
| Q8TA86 | RP9 | 0\|0\|2 | 2 | 0.67 | 2\|2\|2 | 0 | 0 | 0 | 0 | 0 | −10.49 | 0.33 | 0.92 |
| P04792 | HSPB1 | 18\|17\|19 | 54 | 18 | 18\|20\|15 | 0 | 0 | 0 | 0 | 0 | −40.42 | 1.02 | 0.91 |
| O43684-2 | BUB3 | 12\|11\|12 | 35 | 11.67 | 16\|13\|11 | 0 | 0 | 0 | 0 | 0 | −33.97 | 0.87 | 0.92 |
| Q14203-3 | DCTN1 | 14\|12\|14 | 40 | 13.33 | 16\|13\|13 | 0 | 0 | 0 | 0 | 0 | −34.81 | 0.95 | 0.92 |
| Q09666 | AHNAK | 157\|168\|173 | 498 | 166 | 209\|239\| | 0 | 0 | 0 | 0 | 0 | −561.04 | 0.75 | 0.92 |
| | | | | | 213 | | | | | | | | |
| Q15424-2 | SAFB | 3\|5\|2 | 10 | 3.33 | 9\|7\|9 | 0 | 0 | 0 | 0 | 0 | −31.96 | 0.4 | 0.92 |
| Q7LBC6 | KDM3B | 2\|0\|3 | 5 | 1.67 | 5\|3\|3 | 0 | 0 | 0 | 0 | 0 | −17.76 | 0.45 | 0.92 |
| O15091-2 | KIAA0391 | 2\|0\|0 | 2 | 0.67 | 0\|2\|2 | 0 | 0.01 | 0 | 0.01 | 0 | −7.85 | 0.5 | 0.82 |
| Q9BUK6-7 | MSTO1 | 2\|0\|0 | 2 | 0.67 | 2\|2\|0 | 0 | 0.01 | 0 | 0.01 | 0 | −7.85 | 0.5 | 0.82 |
| P55209-3 | NAP1L1 | 3\|3\|3 | 9 | 3 | 0\|3\|3 | 0 | 0 | 0 | 0 | 0 | −5.41 | 1.5 | 0.81 |
| P14625 | HSP90B1 | 6\|4\|10 | 20 | 6.67 | 7\|6\|6 | 0 | 0 | 0 | 0 | 0 | −19.6 | 1.05 | 0.89 |
| P62140 | PPP1CB | 9\|8\|8 | 25 | 8.33 | 7\|9\|9 | 0 | 0 | 0 | 0 | 0 | −20.58 | 1 | 0.91 |
| P13929-3 | ENO3 | 6\|6\|5 | 17 | 5.67 | 3\|6\|6 | 0 | 0 | 0 | 0 | 0 | −13.18 | 1.13 | 0.89 |
| Q6UWE0 | LRSAM1 | 4\|6\|5 | 15 | 5 | 9\|11\|5 | 0 | 0 | 0 | 0 | 0 | −27.42 | 0.6 | 0.92 |
| Q969U7-2 | PSMG2 | 3\|2\|4 | 9 | 3 | 4\|4\|3 | 0 | 0 | 0 | 0 | 0 | −13.04 | 0.82 | 0.88 |
| Q96SB4-4 | SRPK1 | 3\|0\|4 | 7 | 2.33 | 4\|3\|5 | 0 | 0 | 0 | 0 | 0 | −19.16 | 0.58 | 0.92 |
| Q86TX2 | ACOT1 | 0\|0\|2 | 2 | 0.67 | 4\|3\|2 | 0 | 0 | 0 | 0 | 0 | −14.8 | 0.22 | 0.92 |
| Q6RFH5 | WDR74 | 0\|0\|2 | 2 | 0.67 | 3\|8\|4 | 0 | 0 | 0 | 0 | 0 | −23.49 | 0.13 | 0.92 |
| P09651-3 | HNRNPA1 | 3\|5\|5 | 13 | 4.33 | 6\|8\|12 | 0 | 0 | 0 | 0 | 0 | −30.96 | 0.5 | 0.92 |
| Q8WUM0 | NUP133 | 6\|6\|4 | 16 | 5.33 | 6\|7\|6 | 0 | 0 | 0 | 0 | 0 | −19.6 | 0.84 | 0.92 |
| P07237 | P4HB | 4\|5\|4 | 13 | 4.33 | 2\|4\|3 | 0 | 0 | 0 | 0 | 0 | −7.59 | 1.44 | 0.84 |
| P47756-2 | CAPZB | 6\|9\|10 | 25 | 8.33 | 13\|7\|11 | 0 | 0 | 0 | 0 | 0 | −31.29 | 0.81 | 0.92 |
| Q9H9A6 | LRRC40 | 10\|10\|11 | 31 | 10.33 | 14\|7\|11 | 0 | 0 | 0 | 0 | 0 | −25.94 | 0.97 | 0.91 |
| P11387 | TOP1 | 4\|5\|5 | 14 | 4.67 | 7\|4\|4 | 0 | 0 | 0 | 0 | 0 | −14.62 | 0.93 | 0.92 |
| Q9UG63 | ABCF2 | 12\|12\|17 | 41 | 13.67 | 10\|13\|11 | 0 | 0 | 0 | 0 | 0 | −25.35 | 1.21 | 0.9 |
| P04899 | GNAI2 | 3\|4\|4 | 11 | 3.67 | 4\|4\|3 | 0 | 0 | 0 | 0 | 0 | −11.41 | 1 | 0.88 |
| P06493 | CDK1 | 14\|13\|14 | 41 | 13.67 | 9\|11\|6 | 0 | 0 | 0 | 0 | 0 | −14.63 | 1.58 | 0.89 |
| Q02978-2 | SLC25A11 | 0\|0\|3 | 3 | 1 | 3\|2\|2 | 0 | 0 | 0 | 0 | 0 | −11.98 | 0.43 | 0.85 |
| P11766 | ADH5 | 0\|4\|0 | 4 | 1.33 | 2\|3\|2 | 0 | 0.01 | 0 | 0.01 | 0 | −11.98 | 0.57 | 0.83 |
| O43491-4 | EPB41L2 | 4\|3\|2 | 9 | 3 | 4\|3\|4 | 0 | 0 | 0 | 0 | 0 | −13.04 | 0.82 | 0.88 |
| P62847-2 | RPS24 | 7\|5\|5 | 17 | 5.67 | 7\|4\|6 | 0 | 0 | 0 | 0 | 0 | −15.55 | 1 | 0.89 |
| P05787 | KRT8 | 61\|52\|57 | 170 | 56.67 | 47\|56\|53 | 0 | 0 | 0 | 0 | 0 | −110.66 | 1.09 | 0.92 |
| P05783 | KRT18 | 41\|47\|45 | 133 | 44.33 | 34\|41\|34 | 0 | 0 | 0 | 0 | 0 | −71.45 | 1.22 | 0.92 |
| P11169 | SLC2A3 | 2\|0\|0 | 2 | 0.67 | 2\|0\|3 | 0 | 0 | 0 | 0 | 0 | −9.18 | 0.4 | 0.84 |
| Q9Y520-4 | PRRC2C | 5\|6\|5 | 16 | 5.33 | 11\|5\|12 | 0 | 0 | 0 | 0 | 0 | −29.21 | 0.57 | 0.92 |
| P47914 | RPL29 | 3\|4\|5 | 12 | 4 | 4\|4\|4 | 0 | 0 | 0 | 0 | 0 | −12.55 | 1 | 0.88 |
| Q8WX19 | GATAD2B | 2\|4\|0 | 6 | 2 | 5\|4\|5 | 0 | 0 | 0 | 0 | 0 | −22.13 | 0.43 | 0.92 |
| O43318-2 | MAP3K7 | 4\|0\|4 | 8 | 2.67 | | 0 | 0 | 0 | 0 | 0 | −13.27 | 1 | 0.84 |
| P61513 | RPL37A | 3\|2\|3 | 8 | 2.67 | 3\|2\|3 | 0 | 0 | 0 | 0 | 0 | −9.34 | 1 | 0.86 |
| Q9UBQ0 | VPS29 | 0\|2\|2 | 4 | 1.33 | 2\|2\|2 | 0 | 0 | 0 | 0 | 0 | −10.49 | 0.67 | 0.92 |
| Q9NQT5-2 | EXOSC3 | 3\|0\|0 | 3 | 1 | 3\|2\|3 | 0 | 0 | 0 | 0 | 0 | −13.44 | 0.38 | 0.87 |
| Q13546 | RIPK1 | 4\|2\|0 | 6 | 2 | 0\|4\|4 | 0 | 0 | 0 | 0 | 0 | −13.27 | 0.75 | 0.85 |
| Q13547 | HDAC1 | 2\|3\|4 | 9 | 3 | 6\|4\|3 | 0 | 0 | 0 | 0 | 0 | −15.6 | 0.69 | 0.92 |
| P62241 | RPS8 | 12\|11\|15 | 38 | 12.67 | 16\|15\|15 | 0 | 0 | 0 | 0 | 0 | −41.38 | 0.83 | 0.92 |
| P62244 | RPS15A | 9\|12\|13 | 34 | 11.33 | 11\|11\|13 | 0 | 0 | 0 | 0 | 0 | −31.08 | 0.97 | 0.91 |
| P50454 | SERPINH1 | 12\|17\|16 | 45 | 15 | 18\|16\|16 | 0 | 0 | 0 | 0 | 0 | −44.68 | 0.9 | 0.91 |
| P62249 | RPS16 | 12\|11\|14 | 37 | 12.33 | 12\|12\|12 | 0 | 0 | 0 | 0 | 0 | −29.15 | 1.03 | 0.91 |
| Q9Y316-2 | MEMO1 | 0\|2\|2 | 4 | 1.33 | 0\|2\|4 | 0 | 0 | 0 | 0 | 0 | −10.24 | 0.67 | 0.92 |
| P12236 | SLC25A6 | 15\|10\|11 | 36 | 12 | 12\|14\|12 | 0 | 0 | 0 | 0 | 0 | −33.13 | 0.95 | 0.91 |
| P12235 | SLC25A4 | 9\|6\|7 | 22 | 7.33 | 6\|8\|6 | 0 | 0 | 0 | 0 | 0 | −17.62 | 1.1 | 0.9 |
| Q96J01 | THOC3 | 3\|3\|0 | 6 | 2 | 4\|3\|3 | 0 | 0 | 0 | 0 | 0 | −16.29 | 0.6 | 0.92 |
| Q9NP64-2 | ZCCHC17 | 3\|3\|4 | 10 | 3.33 | 6\|8\|7 | 0 | 0 | 0 | 0 | 0 | −24.11 | 0.48 | 0.92 |
| O60506-4 | SYNCRIP | 5\|5\|5 | 15 | 5 | 7\|8\|10 | 0 | 0 | 0 | 0 | 0 | −25.43 | 0.6 | 0.92 |
| Q8IY81 | FTSJ3 | 2\|0\|0 | 2 | 0.67 | 2\|3\|2 | 0 | 0 | 0 | 0 | 0 | −11.98 | 0.29 | 0.92 |
| Q09028-4 | RBBP4 | 3\|0\|2 | 5 | 1.67 | 5\|6\|4 | 0 | 0 | 0 | 0 | 0 | −23.55 | 0.33 | 0.92 |
| O94986-3 | CEP152 | 0\|2\|3 | 5 | 1.67 | 2\|2\|4 | 0 | 0 | 0 | 0 | 0 | −13.44 | 0.63 | 0.87 |
| P34931 | HSPA1L | 20\|16\|16 | 52 | 17.33 | 13\|15\|15 | 0 | 0 | 0 | 0 | 0 | −30.13 | 1.21 | 0.91 |
| Q96EN8 | MOCOS | 3\|5\|5 | 13 | 4.33 | 3\|5\|4 | 0 | 0 | 0 | 0 | 0 | −12.55 | 1.08 | 0.88 |
| P51149 | RAB7A | 13\|11\|11 | 35 | 11.67 | 13\|11\|14 | 0 | 0 | 0 | 0 | 0 | −31.55 | 0.92 | 0.91 |
| P51148 | RAB5C | 6\|4\|7 | 17 | 5.67 | 9\|7\|9 | 0 | 0 | 0 | 0 | 0 | −27.37 | 0.68 | 0.92 |
| P05166 | PCCB | 9\|9\|10 | 28 | 9.33 | 24\|22\|20 | 0 | 0 | 0 | 0 | 0 | −71.6 | 0.42 | 0.92 |
| P05165 | PCCA | 130\|120\|122 | 372 | 124 | 175\|170\| | 0 | 0 | 0 | 0 | 0 | −459.64 | 0.7 | 0.92 |
| | | | | | 184 | | | | | | | | |
| P53621 | COPA | 17\|13\|23 | 53 | 17.67 | 24\|16\|21 | 0 | 0 | 0 | 0 | 0 | −56.84 | 0.87 | 0.92 |
| O95573 | ACSL3 | 7\|6\|14 | 27 | 9 | 13\|11\|9 | 0 | 0 | 0 | 0 | 0 | −33.87 | 0.82 | 0.91 |
| P10412 | HIST1H1E | 3\|6\|4 | 13 | 4.33 | 8\|6\|3 | 0 | 0 | 0 | 0 | 0 | −18.92 | 0.76 | 0.9 |
| Q9UHD8-7 | 9-Sep | 12\|7\|13 | 32 | 10.67 | 10\|11\|15 | 0 | 0 | 0 | 0 | 0 | −35.85 | 0.89 | 0.91 |
| Q9P2E9 | RRBP1 | 4\|4\|12 | 20 | 6.67 | 10\|9\|10 | 0 | 0 | 0 | 0 | 0 | −32.71 | 0.69 | 0.91 |
| P30050 | RPL12 | 10\|7\|9 | 26 | 8.67 | 6\|5\|9 | 0 | 0 | 0 | 0 | 0 | −16.16 | 1.3 | 0.89 |
| P35998 | PSMC2 | 18\|16\|18 | 52 | 17.33 | 14\|20\|17 | 0 | 0 | 0 | 0 | 0 | −39.52 | 1.02 | 0.91 |
| P55795 | HNRNPH2 | 23\|18\|23 | 64 | 21.33 | 25\|25\|33 | 0 | 0 | 0 | 0 | 0 | −75.83 | 0.77 | 0.92 |
| P55084-2 | HADHB | 5\|4\|2 | 11 | 3.67 | 3\|3\|4 | 0 | 0 | 0 | 0 | 0 | −11.76 | 1.1 | 0.86 |
| P08729 | KRT7 | 37\|37\|42 | 116 | 38.67 | 45\|43\|41 | 0 | 0 | 0 | 0 | 0 | −101.02 | 0.9 | 0.92 |
| Q9NNW5 | WDR6 | 0\|0\|2 | 2 | 0.67 | 3\|2\|2 | 0 | 0 | 0 | 0 | 0 | −11.98 | 0.29 | 0.92 |
| Q07157-2 | TJP1 | 0\|0\|2 | 2 | 0.67 | 0\|3\|3 | 0 | 0 | 0 | 0 | 0 | −10.39 | 0.33 | 0.92 |
| Q9HAU0-5 | PLEKHA5 | 0\|0\|2 | 2 | 0.67 | 2\|4\|3 | 0 | 0 | 0 | 0 | 0 | −14.8 | 0.22 | 0.92 |
| Q13409-3 | DYNC112 | 3\|7\|7 | 17 | 5.67 | 5\|6\|8 | 0 | 0 | 0 | 0 | 0 | −21.48 | 0.89 | 0.9 |
| O14979 | HNRNPDL | 5\|5\|5 | 15 | 5 | 6\|5\|8 | 0 | 0 | 0 | 0 | 0 | −17.93 | 0.79 | 0.92 |
| Q86UK7-2 | ZNF598 | 6\|6\|9 | 21 | 7 | 9\|9\|6 | 0 | 0 | 0 | 0 | 0 | −22.42 | 0.88 | 0.9 |
| Q8WWI1-3 | LM07 | 5\|4\|8 | 17 | 5.67 | 10\|12\|15 | 0 | 0 | 0 | 0 | 0 | −43.61 | 0.46 | 0.92 |
| Q99567 | NUP88 | 5\|3\|6 | 14 | 4.67 | 5\|9\|9 | 0 | 0 | 0 | 0 | 0 | −26.82 | 0.61 | 0.92 |
| O15144 | ARPC2 | 4\|3\|3 | 10 | 3.33 | 3\|5\|4 | 0 | 0 | 0 | 0 | 0 | −12.55 | 0.83 | 0.92 |
| O15145 | ARPC3 | 4\|3\|3 | 10 | 3.33 | 4\|5\|3 | 0 | 0 | 0 | 0 | 0 | −12.55 | 0.83 | 0.92 |
| Q96KP4 | CNDP2 | 8\|6\|8 | 22 | 7.33 | 6\|7\|6 | 0 | 0 | 0 | 0 | 0 | −16.44 | 1.16 | 0.89 |
| Q9Y266 | NUDC | 2\|2\|3 | 7 | 2.33 | 3\|0\|3 | 0 | 0 | 0 | 0 | 0 | −6.87 | 1.17 | 0.83 |
| Q9Y265 | RUVBL1 | 6\|6\|6 | 18 | 6 | 6\|4\|4 | 0 | 0 | 0 | 0 | 0 | −10.57 | 1.29 | 0.88 |
| Q9Y263 | PLAA | 13\|6\|11 | 30 | 10 | 12\|10\|11 | 0 | 0 | 0 | 0 | 0 | −33.87 | 0.91 | 0.91 |
| Q08211 | DHX9 | 33\|33\|35 | 101 | 33.67 | 43\|43\|44 | 0 | 0 | 0 | 0 | 0 | −108.8 | 0.78 | 0.92 |
| Q5TFE4 | NT5DC1 | 3\|3\|4 | 10 | 3.33 | 6\|7\|9 | 0 | 0 | 0 | 0 | 0 | −25.47 | 0.45 | 0.92 |
| Q8TAT6 | NPLOC4 | 11\|7\|4 | 22 | 7.33 | 4\|9\|7 | 0 | 0 | 0 | 0 | 0 | −20.88 | 1.1 | 0.89 |
| P21333-2 | FLNA | 49\|34\|37 | 120 | 40 | 55\|48\|50 | 0 | 0 | 0 | 0 | 0 | −136.08 | 0.78 | 0.92 |
| P46777 | RPL5 | 28\|32\|36 | 96 | 32 | 33\|35\|29 | 0 | 0 | 0 | 0 | 0 | −76.37 | 0.99 | 0.92 |
| O43823 | AKAP8 | 6\|2\|3 | 11 | 3.67 | 8\|6\|5 | 0 | 0 | 0 | 0 | 0 | −23.65 | 0.58 | 0.92 |
| Q01085-2 | TIAL1 | 11\|6\|7 | 24 | 8 | 7\|7\|6 | 0 | 0 | 0 | 0 | 0 | −17.62 | 1.2 | 0.89 |
| Q96RT1-7 | ERBIN | 0\|2\|0 | 2 | 0.67 | 2\|0\|3 | 0 | 0 | 0 | 0 | 0 | −9.18 | 0.4 | 0.84 |
| Q99798 | ACO2 | 6\|2\|0 | 8 | 2.67 | 10\|7\|9 | 0 | 0 | 0 | 0 | 0 | −39.81 | 0.31 | 0.92 |
| Q13148 | TARDBP | 6\|5\|9 | 20 | 6.67 | 9\|11\|10 | 0 | 0 | 0 | 0 | 0 | −31.92 | 0.67 | 0.92 |
| Q13144 | EIF2B5 | 2\|2\|0 | 4 | 1.33 | 2\|2\|3 | 0 | 0 | 0 | 0 | 0 | −11.98 | 0.57 | 0.92 |
| P35237 | SERPINB6 | 6\|6\|8 | 20 | 6.67 | 8\|4\|7 | 0 | 0 | 0 | 0 | 0 | −16.44 | 1.05 | 0.9 |
| Q92785 | DPF2 | 2\|0\|0 | 2 | 0.67 | 0\|2\|2 | 0 | 0.01 | 0 | 0.01 | 0 | −7.85 | 0.5 | 0.82 |
| P52789 | HK2 | 4\|3\|4 | 11 | 3.67 | 6\|5\|5 | 0 | 0 | 0 | 0 | 0 | −17.58 | 0.69 | 0.92 |
| P54578-2 | USP14 | 6\|4\|4 | 14 | 4.67 | 6\|4\|5 | 0 | 0 | 0 | 0 | 0 | −14.62 | 0.93 | 0.89 |
| Q01469 | FABP5 | 4\|2\|5 | 11 | 3.67 | 3\|3\|3 | 0 | 0 | 0 | 0 | 0 | −10.47 | 1.22 | 0.85 |
| P62888 | RPL30 | 7\|4\|6 | 17 | 5.67 | 8\|7\|7 | 0 | 0 | 0 | 0 | 0 | −23.44 | 0.77 | 0.92 |
| O43175 | PHGDH | 2\|2\|2 | 6 | 2 | 3\|2\|2 | 0 | 0 | 0 | 0 | 0 | −8.15 | 0.86 | 0.92 |
| Q8IYW5 | RNF168 | 0\|0\|2 | 2 | 0.67 | 2\|3\|0 | 0 | 0 | 0 | 0 | 0 | −9.18 | 0.4 | 0.84 |
| P52209-2 | PGD | 4\|4\|0 | 8 | 2.67 | 3\|2\|7 | 0 | 0 | 0 | 0 | 0 | −18.99 | 0.67 | 0.92 |
| P26599 | PTBP1 | 8\|7\|10 | 25 | 8.33 | 9\|11\|14 | 0 | 0 | 0 | 0 | 0 | −33.29 | 0.74 | 0.92 |
| O43395 | PRPF3 | 2\|0\|0 | 2 | 0.67 | 6\|4\|4 | 0 | 0 | 0 | 0 | 0 | −22.13 | 0.14 | 0.92 |
| Q13557-12 | CAMK2D | 0\|0\|2 | 2 | 0.67 | 2\|2\|2 | 0 | 0 | 0 | 0 | 0 | −10.49 | 0.33 | 0.92 |
| P12956 | XRCC6 | 24\|26\|23 | 73 | 24.33 | 30\|27\|26 | 0 | 0 | 0 | 0 | 0 | −67.26 | 0.88 | 0.92 |
| P23526 | AHCY | 33\|33\|35 | 101 | 33.67 | 32\|36\|36 | 0 | 0 | 0 | 0 | 0 | −77.14 | 0.97 | 0.92 |
| P23527 | HIST1H2BO | 3\|3\|5 | 11 | 3.67 | 7\|9\|6 | 0 | 0 | 0 | 0 | 0 | −25.47 | 0.5 | 0.92 |
| P09874 | PARP1 | 31\|24\|30 | 85 | 28.33 | 44\|38\|42 | 0 | 0 | 0 | 0 | 0 | −117.49 | 0.69 | 0.92 |
| P23528 | CFL1 | 14\|11\|11 | 36 | 12 | 12\|12\|18 | 0 | 0 | 0 | 0 | 0 | −36.4 | 0.86 | 0.92 |
| Q96PK6-5 | RBM14 | 5\|6\|6 | 17 | 5.67 | 7\|7\|6 | 0 | 0 | 0 | 0 | 0 | −19.16 | 0.85 | 0.92 |
| P19388 | POLR2E | 2\|0\|2 | 4 | 1.33 | 0\|3\|3 | 0 | 0 | 0 | 0 | 0 | −10.39 | 0.67 | 0.92 |
| O14828-2 | SCAMP3 | 6\|0\|3 | 9 | 3 | 8\|6\|9 | 0 | 0 | 0 | 0 | 0 | −35.37 | 0.39 | 0.92 |
| Q14697 | GANAB | 13\|19\|15 | 47 | 15.67 | 18\|19\|16 | 0 | 0 | 0 | 0 | 0 | −46.72 | 0.89 | 0.91 |
| P19387 | POLR2C | 5\|4\|5 | 14 | 4.67 | 4\|3\|3 | 0 | 0 | 0 | 0 | 0 | −8.76 | 1.4 | 0.85 |
| Q9NW64 | RBM22 | 4\|6\|3 | 13 | 4.33 | 4\|7\|8 | 0 | 0 | 0 | 0 | 0 | −21.48 | 0.68 | 0.92 |
| P37802 | TAGLN2 | 17\|18\|16 | 51 | 17 | 19\|18\|20 | 0 | 0 | 0 | 0 | 0 | −46.71 | 0.89 | 0.92 |
| Q93009-3 | USP7 | 6\|5\|9 | 20 | 6.67 | 8\|9\|10 | 0 | 0 | 0 | 0 | 0 | −28 | 0.74 | 0.92 |
| P36507 | MAP2K2 | 2\|2\|4 | 8 | 2.67 | 3\|3\|2 | 0 | 0 | 0 | 0 | 0 | −9.34 | 1 | 0.85 |
| Q15654 | TRIP6 | 9\|5\|14 | 28 | 9.33 | 9\|10\|10 | 0 | 0 | 0 | 0 | 0 | −30.63 | 0.97 | 0.9 |
| P36873 | PPP1CC | 8\|7\|7 | 22 | 7.33 | 6\|8\|10 | 0 | 0 | 0 | 0 | 0 | −20.85 | 0.92 | 0.92 |
| Q9UPT8 | ZC3H4 | 4\|4\|4 | 12 | 4 | 6\|4\|3 | 0 | 0 | 0 | 0 | 0 | −12.29 | 0.92 | 0.92 |
| Q3MHD2 | LSM12 | 2\|3\|3 | 8 | 2.67 | 3\|2\|3 | 0 | 0 | 0 | 0 | 0 | −9.34 | 1 | 0.86 |
| Q6DD87 | ZNF787 | 0\|0\|2 | 2 | 0.67 | 5\|0\|2 | 0 | 0 | 0 | 0 | 0 | −11.64 | 0.29 | 0.92 |
| Q6DD88 | ATL3 | 2\|0\|0 | 2 | 0.67 | 3\|0\|3 | 0 | 0 | 0 | 0 | 0 | −10.39 | 0.33 | 0.92 |
| Q9Y5Q8-2 | GTF3C5 | 0\|0\|2 | 2 | 0.67 | 5\|5\|4 | 0 | 0 | 0 | 0 | 0 | −22.13 | 0.14 | 0.92 |
| Q5GLZ8-6 | HERC4 | 0\|2\|0 | 2 | 0.67 | 0\|2\|2 | 0 | 0.01 | 0 | 0.01 | 0 | −7.85 | 0.5 | 0.82 |
| Q9BRA2 | TXNDC17 | 0\|2\|0 | 2 | 0.67 | 2\|3\|2 | 0 | 0 | 0 | 0 | 0 | −11.98 | 0.29 | 0.92 |
| Q8WXF1 | PSPC1 | 4\|5\|4 | 13 | 4.33 | 2\|3\|5 | 0 | 0 | 0 | 0 | 0 | −8.76 | 1.3 | 0.86 |
| Q9BQ04 | RBM4B | 4\|2\|2 | 8 | 2.67 | 4\|2\|5 | 0 | 0 | 0 | 0 | 0 | −13.04 | 0.73 | 0.88 |
| Q9UBU8-2 | MORF4L1 | 4\|4\|3 | 11 | 3.67 | 2\|3\|3 | 0 | 0 | 0 | 0 | 0 | −7.86 | 1.38 | 0.84 |
| P22695 | UQCRC2 | 6\|7\|4 | 17 | 5.67 | 11\|10\|11 | 0 | 0 | 0 | 0 | 0 | −36.77 | 0.53 | 0.92 |
| P22694 | PRKACB | 4\|3\|0 | 7 | 2.33 | 2\|4\|3 | 0 | 0 | 0 | 0 | 0 | −14.8 | 0.78 | 0.86 |
| Q16555-2 | DPYSL2 | 7\|10\|9 | 26 | 8.67 | 5\|8\|6 | 0 | 0 | 0 | 0 | 0 | −14.98 | 1.37 | 0.89 |
| P11021 | HSPA5 | 18\|11\|11 | 40 | 13.33 | 14\|19\|16 | 0 | 0 | 0 | 0 | 0 | −45.17 | 0.82 | 0.91 |
| Q13620-1 | CUL4B | 3\|0\|3 | 6 | 2 | 0\|5\|5 | 0 | 0 | 0 | 0 | 0 | −15.97 | 0.6 | 0.92 |
| Q96CW1-2 | AP2M1 | 4\|2\|5 | 11 | 3.67 | 4\|2\|4 | 0 | 0 | 0 | 0 | 0 | −11.76 | 1.1 | 0.86 |
| Q13885 | TUBB2A | 86\|88\|87 | 261 | 87 | 100\|93\|91 | 0 | 0 | 0 | 0 | 0 | −212.04 | 0.92 | 0.92 |
| O00148 | DDX39A | 13\|13\|9 | 35 | 11.67 | 12\|12\|12 | 0 | 0 | 0 | 0 | 0 | −32.29 | 0.97 | 0.91 |
| Q9BW19 | KIFC1 | 4\|2\|2 | 8 | 2.67 | 2\|4\|3 | 0 | 0 | 0 | 0 | 0 | −10.47 | 0.89 | 0.86 |
| P48643 | CCT5 | 33\|30\|31 | 94 | 31.33 | 35\|43\|44 | 0 | 0 | 0 | 0 | 0 | −103.93 | 0.77 | 0.92 |
| Q15942 | ZYX | 4\|5\|8 | 17 | 5.67 | 6\|7\|6 | 0 | 0 | 0 | 0 | 0 | −19.6 | 0.89 | 0.9 |
| P49790 | NUP153 | 24\|23\|25 | 72 | 24 | 27\|29\|26 | 0 | 0 | 0 | 0 | 0 | −66.04 | 0.88 | 0.92 |
| Q14807-2 | KIF22 | 0\|3\|4 | 7 | 2.33 | 5\|4\|3 | 0 | 0 | 0 | 0 | 0 | −19.16 | 0.58 | 0.92 |
| O75179-7 | ANKRD17 | 8\|5\|11 | 24 | 8 | 6\|11\|10 | 0 | 0 | 0 | 0 | 0 | −28 | 0.89 | 0.91 |
| P30041 | PRDX6 | 8\|8\|9 | 25 | 8.33 | 7\|6\|6 | 0 | 0 | 0 | 0 | 0 | −13.55 | 1.32 | 0.89 |
| Q8TEU7-5 | RAPGEF6 | 0\|0\|2 | 2 | 0.67 | 2\|2\|2 | 0 | 0 | 0 | 0 | 0 | −10.49 | 0.33 | 0.92 |
| Q9Y678 | COPG1 | 19\|16\|13 | 48 | 16 | 13\|12\|14 | 0 | 0 | 0 | 0 | 0 | −29.78 | 1.23 | 0.91 |
| Q9Y679 | AUP1 | 5\|3\|0 | 8 | 2.67 | 4\|7\|4 | 0 | 0 | 0 | 0 | 0 | −23.55 | 0.53 | 0.92 |
| P50579-3 | METAP2 | 4\|0\|4 | 8 | 2.67 | 3\|3\|4 | 0 | 0 | 0 | 0 | 0 | −16.29 | 0.8 | 0.87 |
| P55786 | NPEPPS | 2\|4\|3 | 9 | 3 | 4\|4\|4 | 0 | 0 | 0 | 0 | 0 | −14.26 | 0.75 | 0.92 |
| P37837 | TALDO1 | 4\|3\|5 | 12 | 4 | 2\|4\|2 | 0 | 0.01 | 0 | 0.01 | 0 | −7.86 | 1.5 | 0.83 |
| Q16637-4 | SMN2; | 0\|3\|2 | 5 | 1.67 | 4\|3\|0 | 0 | 0 | 0 | 0 | 0 | −11.81 | 0.71 | 0.85 |
| | SMN1 | | | | | | | | | | | | |
| Q9NP72 | RAB18 | 2\|3\|4 | 9 | 3 | 4\|4\|5 | 0 | 0 | 0 | 0 | 0 | −15.6 | 0.69 | 0.92 |
| Q9NP77 | SSU72 | 2\|0\|0 | 2 | 0.67 | 2\|4\|4 | 0 | 0 | 0 | 0 | 0 | −16.29 | 0.2 | 0.92 |
| P62879 | GNB2 | 3\|3\|5 | 11 | 3.67 | 3\|3\|5 | 0 | 0 | 0 | 0 | 0 | −11.41 | 1 | 0.87 |
| Q9NRR4-2 | DROSHA | 3\|4\|3 | 10 | 3.33 | 3\|2\|3 | 0 | 0 | 0 | 0 | 0 | −7.86 | 1.25 | 0.85 |
| Q15003 | NCAPH | 9\|9\|4 | 22 | 7.33 | 6\|5\|6 | 0 | 0 | 0 | 0 | 0 | −17.11 | 1.29 | 0.88 |
| Q9C0C2 | TNKS1BP1 | 3\|3\|4 | 10 | 3.33 | 6\|4\|4 | 0 | 0 | 0 | 0 | 0 | −15.06 | 0.71 | 0.92 |
| Q15796-2 | SMAD2 | 2\|0\|0 | 2 | 0.67 | 2\|2\|2 | 0 | 0 | 0 | 0 | 0 | −10.49 | 0.33 | 0.92 |
| Q6KB66-2 | KRT80 | 2\|0\|0 | 2 | 0.67 | 3\|2\|0 | 0 | 0 | 0 | 0 | 0 | −9.18 | 0.4 | 0.84 |
| Q9UKG1 | APPL1 | 4\|3\|4 | 11 | 3.67 | 4\|2\|5 | 0 | 0 | 0 | 0 | 0 | −11.41 | 1 | 0.88 |
| Q06210-2 | GFPT1 | 13\|16\|20 | 49 | 16.33 | 18\|18\|19 | 0 | 0 | 0 | 0 | 0 | −49.22 | 0.89 | 0.91 |
| P18085 | ARF4 | 6\|9\|7 | 22 | 7.33 | 8\|3\|8 | 0 | 0 | 0 | 0 | 0 | −16.41 | 1.16 | 0.89 |
| O75821 | EIF3G | 2\|0\|0 | 2 | 0.67 | 2\|2\|4 | 0 | 0 | 0 | 0 | 0 | −13.44 | 0.25 | 0.92 |
| P30044-2 | PRDX5 | 5\|3\|4 | 12 | 4 | 3\|5\|4 | 0 | 0 | 0 | 0 | 0 | −12.55 | 1 | 0.88 |
| P78371-2 | CCT2 | 3\|2\|0 | 5 | 1.67 | 3\|3\|4 | 0 | 0 | 0 | 0 | 0 | −16.29 | 0.5 | 0.92 |
| Q9UGI8-2 | TES | 7\|9\|15 | 31 | 10.33 | 13\|11\|10 | 0 | 0 | 0 | 0 | 0 | −33.29 | 0.91 | 0.91 |
| Q5VV41 | ARHGEF16 | 6\|5\|7 | 18 | 6 | 7\|6\|10 | 0 | 0 | 0 | 0 | 0 | −22.9 | 0.78 | 0.92 |
| Q5VV42 | CDKAL1 | 2\|0\|0 | 2 | 0.67 | 3\|3\|0 | 0 | 0 | 0 | 0 | 0 | −10.39 | 0.33 | 0.92 |
| P04222 | HLA-C | 2\|3\|0 | 5 | 1.67 | 3\|5\|3 | 0 | 0 | 0 | 0 | 0 | −17.76 | 0.45 | 0.92 |
| P68104 | EEF1A1 | 68\|59\|64 | 191 | 63.67 | 74\|94\|97 | 0 | 0 | 0 | 0 | 0 | −233.41 | 0.72 | 0.92 |
| P27635 | RPL10 | 18\|18\|20 | 56 | 18.67 | 21\|22\|19 | 0 | 0 | 0 | 0 | 0 | 49.65 | 0.9 | 0.92 |
| Q9UHB9-4 | SRP68 | 3\|2\|3 | 8 | 2.67 | 3\|2\|4 | 0 | 0 | 0 | 0 | 0 | −10.47 | 0.89 | 0.92 |
| Q9Y2H6-2 | FNDC3A | 0\|3\|4 | 7 | 2.33 | 4\|2\|9 | 0 | 0 | 0 | 0 | 0 | −23.18 | 0.47 | 0.92 |
| Q9Y450 | HBS1L | 8\|4\|5 | 17 | 5.67 | 8\|10\|7 | 0 | 0 | 0 | 0 | 0 | −27.37 | 0.68 | 0.92 |
| Q92804-2 | TAF15 | 4\|4\|12 | 20 | 6.67 | 15\|12\|10 | 0 | 0 | 0 | 0 | 0 | −43.61 | 0.54 | 0.92 |
| Q5BKZ1 | ZNF326 | 3\|2\|3 | 8 | 2.67 | 5\|2\|5 | 0 | 0 | 0 | 0 | 0 | −14.26 | 0.67 | 0.92 |
| Q6L8Q7-2 | PDE12 | 5\|4\|5 | 14 | 4.67 | 7\|6\|8 | 0 | 0 | 0 | 0 | 0 | −22.13 | 0.67 | 0.92 |
| Q8NFH4 | NUP37 | 0\|0\|3 | 3 | 1 | 0\|3\|3 | 0 | 0 | 0 | 0 | 0 | −10.39 | 0.5 | 0.83 |
| Q13151 | HNRNPAO | 5\|8\|10 | 23 | 7.67 | 7\|7\|7 | 0 | 0 | 0 | 0 | 0 | −20.36 | 1.1 | 0.9 |
| Q13155 | AIMP2 | 2\|0\|2 | 4 | 1.33 | 3\|3\|2 | 0 | 0 | 0 | 0 | 0 | −13.44 | 0.5 | 0.92 |
| Q14192 | FHL2 | 5\|5\|6 | 16 | 5.33 | 6\|5\|5 | 0 | 0 | 0 | 0 | 0 | −14.36 | 1 | 0.9 |
| Q86TC9 | MYPN | 6\|4\|7 | 17 | 5.67 | 3\|4\|3 | 0 | 0.01 | 0 | 0.01 | 0 | −8.76 | 1.7 | 0.81 |
| P62891 | RPL39 | 4\|3\|3 | 10 | 3.33 | 6\|3\|2 | 0 | 0 | 0 | 0 | 0 | −11.42 | 0.91 | 0.89 |
| Q14204 | DYNC1H1 | 21\|21\|31 | 73 | 24.33 | 32\|26\|26 | 0 | 0 | 0 | 0 | 0 | −71.79 | 0.87 | 0.92 |
| P00338 | LDHA | 19\|16\|14 | 49 | 16.33 | 15\|15\|14 | 0 | 0 | 0 | 0 | 0 | −34.2 | 1.11 | 0.91 |
| Q92841 | DDX17 | 48\|47\|49 | 144 | 48 | 54\|53\|50 | 0 | 0 | 0 | 0 | 0 | −119.29 | 0.92 | 0.92 |
| P68032 | ACTC1 | 62\|62\|69 | 193 | 64.33 | 82\|78\|72 | 0 | 0 | 0 | 0 | 0 | −186.85 | 0.83 | 0.92 |
| P07814 | EPRS | 22\|23\|37 | 82 | 27.33 | 40\|30\|35 | 0 | 0 | 0 | 0 | 0 | −96.67 | 0.78 | 0.92 |
| P22102 | GART | 11\|11\|13 | 35 | 11.67 | 8\|9\|12 | 0 | 0 | 0 | 0 | 0 | −20.94 | 1.21 | 0.9 |
| O75643 | SNRNP200 | 5\|3\|5 | 13 | 4.33 | 12\|17\|14 | 0 | 0 | 0 | 0 | 0 | −54.63 | 0.3 | 0.92 |
| Q15185-4 | PTGES3 | 4\|4\|3 | 11 | 3.67 | 4\|4\|5 | 0 | 0 | 0 | 0 | 0 | −13.81 | 0.85 | 0.92 |
| Q8WWY3 | PRPF31 | 3\|2\|3 | 8 | 2.67 | 5\|4\|5 | 0 | 0 | 0 | 0 | 0 | −16.93 | 0.57 | 0.92 |
| O60271-4 | SPAG9 | 5\|5\|5 | 15 | 5 | 8\|6\|10 | 0 | 0 | 0 | 0 | 0 | −24.14 | 0.62 | 0.92 |
| P00761 | P00761 | 26\|28\|22 | 76 | 25.33 | 31\|24\|25 | 0 | 0 | 0 | 0 | 0 | −65.2 | 0.95 | 0.92 |
| Q12931-2 | TRAP1 | 20\|21\|25 | 66 | 22 | 26\|24\|29 | 0 | 0 | 0 | 0 | 0 | −67.26 | 0.84 | 0.92 |
| Q14739 | LBR | 9\|5\|6 | 20 | 6.67 | 9\|8\|7 | 0 | 0 | 0 | 0 | 0 | −24.14 | 0.83 | 0.9 |
| P61106 | RAB14 | 9\|12\|10 | 31 | 10.33 | 14\|16\|18 | 0 | 0 | 0 | 0 | 0 | −47.59 | 0.65 | 0.92 |
| Q9UNQ2 | DIMT1 | 3\|2\|2 | 7 | 2.33 | 3\|2\|3 | 0 | 0 | 0 | 0 | 0 | −9.34 | 0.88 | 0.87 |
| O43491 | EPB41L2 | 5\|4\|4 | 13 | 4.33 | 6\|3\|7 | 0 | 0 | 0 | 0 | 0 | −15.87 | 0.81 | 0.92 |
| P11586 | MTHFD1 | 5\|3\|6 | 14 | 4.67 | 6\|6\|5 | 0 | 0 | 0 | 0 | 0 | −18.88 | 0.82 | 0.9 |
| P61353 | RPL27 | 13\|13\|14 | 40 | 13.33 | 14\|14\|12 | 0 | 0 | 0 | 0 | 0 | −30.95 | 1 | 0.91 |
| O14828 | SCAMP3 | 4\|0\|3 | 7 | 2.33 | 10\|8\|7 | 0 | 0 | 0 | 0 | 0 | −38.32 | 0.28 | 0.92 |
| P38919 | EIF4A3 | 10\|12\|16 | 38 | 12.67 | 18\|24\|24 | 0 | 0 | 0 | 0 | 0 | −69.39 | 0.58 | 0.92 |
| Q15645 | TRIP13 | 6\|9\|8 | 23 | 7.67 | 8\|6\|6 | 0 | 0 | 0 | 0 | 0 | −17.62 | 1.15 | 0.9 |
| Q13423 | NNT | 9\|4\|9 | 22 | 7.33 | 11\|9\|12 | 0 | 0 | 0 | 0 | 0 | −36.77 | 0.69 | 0.92 |
| Q7Z794 | KRT77 | 5\|5\|0 | 10 | 3.33 | 5\|7\|5 | 0 | 0 | 0 | 0 | 0 | −26.53 | 0.59 | 0.92 |
| P35080-2 | PFN2 | 4\|4\|4 | 12 | 4 | 4\|5\|4 | 0 | 0 | 0 | 0 | 0 | −12.29 | 0.92 | 0.92 |
| P62081 | RPS7 | 24\|19\|27 | 70 | 23.33 | 18\|21\|25 | 0 | 0 | 0 | 0 | 0 | −50.51 | 1.09 | 0.91 |
| P48735 | IDH2 | 4\|0\|3 | 7 | 2.33 | 4\|2\|3 | 0 | 0 | 0 | 0 | 0 | −14.8 | 0.78 | 0.86 |
| O15371-2 | EIF3D | 29\|22\|30 | 81 | 27 | 29\|24\|23 | 0 | 0 | 0 | 0 | 0 | −60.33 | 1.07 | 0.92 |
| Q9NR45 | NANS | 0\|0\|2 | 2 | 0.67 | 3\|2\|3 | 0 | 0 | 0 | 0 | 0 | −13.44 | 0.25 | 0.92 |
| P84098 | RPL19 | 8\|10\|13 | 31 | 10.33 | 12\|6\|8 | 0 | 0 | 0 | 0 | 0 | −21.82 | 1.19 | 0.9 |
| Q9Y697-2 | NFS1 | 4\|0\|5 | 9 | 3 | 4\|2\|4 | 0 | 0 | 0 | 0 | 0 | −16.29 | 0.9 | 0.86 |
| P84090 | ERH | 3\|0\|0 | 3 | 1 | 4\|2\|2 | 0 | 0 | 0 | 0 | 0 | −13.44 | 0.38 | 0.87 |
| Q15269 | PWP2 | 3\|4\|3 | 10 | 3.33 | 0\|4\|4 | 0 | 0 | 0 | 0 | 0 | −7.6 | 1.25 | 0.84 |
| P84095 | RHOG | 3\|3\|3 | 9 | 3 | 4\|3\|3 | 0 | 0 | 0 | 0 | 0 | −10.22 | 0.9 | 0.92 |
| P07355 | ANXA2 | 6\|4\|8 | 18 | 6 | 6\|5\|5 | 0 | 0 | 0 | 0 | 0 | −15.87 | 1.12 | 0.89 |
| Q8TDN6 | BRIX1 | 0\|0\|2 | 2 | 0.67 | 2\|2\|0 | 0 | 0.01 | 0 | 0.01 | 0 | −7.85 | 0.5 | 0.82 |
| P62136 | PPP1CA | 10\|10\|10 | 30 | 10 | 9\|9\|11 | 0 | 0 | 0 | 0 | 0 | −22.38 | 1.03 | 0.91 |
| O00487 | PSMD14 | 4\|3\|4 | 11 | 3.67 | 2\|4\|5 | 0 | 0 | 0 | 0 | 0 | −11.41 | 1 | 0.88 |
| Q8NC51-4 | SERBP1 | 4\|4\|6 | 14 | 4.67 | 4\|3\|4 | 0 | 0 | 0 | 0 | 0 | −9.93 | 1.27 | 0.86 |
| P05141 | SLC25A5 | 13\|10\|13 | 36 | 12 | 13\|15\|11 | 0 | 0 | 0 | 0 | 0 | −34.35 | 0.92 | 0.92 |
| Q9UQ80 | PA2G4 | 42\|26\|37 | 105 | 35 | 37\|36\|31 | 0 | 0 | 0 | 0 | 0 | −88.26 | 1.01 | 0.92 |
| O14545 | TRAFD1 | 2\|2\|2 | 6 | 2 | 3\|0\|2 | 0 | 0 | 0 | 0 | 0 | −5.71 | 1.2 | 0.81 |
| Q14683 | SMC1A | 6\|5\|10 | 21 | 7 | 8\|10\|6 | 0 | 0 | 0 | 0 | 0 | −24.14 | 0.88 | 0.9 |
| P04818-2 | TYMS | 4\|3\|5 | 12 | 4 | 4\|4\|3 | 0 | 0 | 0 | 0 | 0 | −11.41 | 1.09 | 0.87 |
| P47712 | PLA2G4A | 4\|2\|4 | 10 | 3.33 | 6\|4\|4 | 0 | 0 | 0 | 0 | 0 | −16.93 | 0.71 | 0.92 |
| Q9BV38 | WDR18 | 8\|5\|5 | 18 | 6 | 4\|6\|5 | 0 | 0 | 0 | 0 | 0 | −13.18 | 1.2 | 0.88 |
| Q96EE3 | SEH1L | 6\|4\|7 | 17 | 5.67 | 5\|7\|6 | 0 | 0 | 0 | 0 | 0 | −18.31 | 0.94 | 0.9 |
| Q9Y666 | SLC12A7 | 2\|2\|3 | 7 | 2.33 | 2\|4\|0 | 0 | 0.01 | 0 | 0.01 | 0 | −6.72 | 1.17 | 0.83 |
| Q9UBE0 | SAE1 | 4\|6\|7 | 17 | 5.67 | 9\|8\|9 | 0 | 0 | 0 | 0 | 0 | −28.7 | 0.65 | 0.92 |
| Q92900-2 | UPF1 | 14\|12\|18 | 44 | 14.67 | 17\|19\|19 | 0 | 0 | 0 | 0 | 0 | −51 | 0.8 | 0.92 |
| Q969S3 | ZNF622 | 6\|5\|8 | 19 | 6.33 | 5\|6\|4 | 0 | 0 | 0 | 0 | 0 | −13.18 | 1.27 | 0.88 |
| Q16891-2 | IMMT | 2\|2\|4 | 8 | 2.67 | 4\|6\|0 | 0 | 0 | 0 | 0 | 0 | −11.39 | 0.8 | 0.87 |
| O00571 | DDX3X | 60\|41\|50 | 151 | 50.33 | 51\|53\|68 | 0 | 0 | 0 | 0 | 0 | −147.58 | 0.88 | 0.92 |
| P14866 | HNRNPL | 30\|34\|39 | 103 | 34.33 | 43\|43\|45 | 0 | 0 | 0 | 0 | 0 | −115.29 | 0.79 | 0.92 |
| P52594-2 | AGFG1 | 3\|5\|5 | 13 | 4.33 | 5\|6\|5 | 0 | 0 | 0 | 0 | 0 | −17.58 | 0.81 | 0.92 |
| P14868 | DARS | 9\|12\|10 | 31 | 10.33 | 13\|14\|10 | 0 | 0 | 0 | 0 | 0 | −33.55 | 0.84 | 0.92 |
| O15027-5 | SEC16A | 14\|11\|15 | 40 | 13.33 | 21\|20\|21 | 0 | 0 | 0 | 0 | 0 | −61.99 | 0.65 | 0.92 |
| Q5T200-2 | ZC3H13 | 8\|4\|7 | 19 | 6.33 | 7\|5\|5 | 0 | 0 | 0 | 0 | 0 | −17.11 | 1.12 | 0.89 |
| P31947-2 | SFN | 3\|2\|2 | 7 | 2.33 | 2\|4\|2 | 0 | 0 | 0 | 0 | 0 | −9.34 | 0.88 | 0.87 |
| Q08123 | NSUN2 | 7\|16\|13 | 36 | 12 | 16\|12\|14 | 0 | 0 | 0 | 0 | 0 | −43.71 | 0.86 | 0.91 |
| P0DPH8 | PODPH8 | 69\|55\|65 | 189 | 63 | 69\|63\|66 | 0 | 0 | 0 | 0 | 0 | −156.46 | 0.95 | 0.92 |
| Q9Y446 | PKP3 | 5\|4\|3 | 12 | 4 | 5\|5\|5 | 0 | 0 | 0 | 0 | 0 | −16.28 | 0.8 | 0.92 |
| O60678-2 | PRMT3 | 5\|6\|4 | 15 | 5 | 4\|2\|5 | 0 | 0 | 0 | 0 | 0 | −9.93 | 1.36 | 0.86 |
| Q969V3-2 | NCLN | 3\|5\|3 | 11 | 3.67 | 4\|5\|2 | 0 | 0 | 0 | 0 | 0 | −11.41 | 1 | 0.87 |
| Q9Y3A5 | SBDS | 10\|10\|11 | 31 | 10.33 | 14\|13\|12 | 0 | 0 | 0 | 0 | 0 | −34.35 | 0.79 | 0.92 |
| P15170-2 | GSPT1 | 5\|5\|6 | 16 | 5.33 | 6\|8\|7 | 0 | 0 | 0 | 0 | 0 | −20.36 | 0.76 | 0.92 |
| P42677 | RPS27 | 7\|7\|7 | 21 | 7 | 11\|7\|6 | 0 | 0 | 0 | 0 | 0 | −20.85 | 0.88 | 0.92 |
| Q04760 | GLO1 | 11\|8\|11 | 30 | 10 | 12\|11\|9 | 0 | 0 | 0 | 0 | 0 | −29.02 | 0.94 | 0.91 |
| Q5VT66-3 | 1-Mar | 5\|3\|5 | 13 | 4.33 | 4\|5\|3 | 0 | 0 | 0 | 0 | 0 | −12.55 | 1.08 | 0.88 |
| Q01082 | SPTBN1 | 4\|6\|5 | 15 | 5 | 7\|8\|7 | 0 | 0 | 0 | 0 | 0 | −23.44 | 0.68 | 0.92 |
| Q01081 | U2AF1 | 3\|9\|7 | 19 | 6.33 | 9\|6\|8 | 0 | 0 | 0 | 0 | 0 | −26.82 | 0.83 | 0.9 |
| P26373 | RPL13 | 25\|27\|28 | 80 | 26.67 | 28\|22\|23 | 0 | 0 | 0 | 0 | 0 | −52.31 | 1.1 | 0.92 |
| P42704 | LRPPRC | 0\|4\|4 | 8 | 2.67 | 4\|7\|4 | 0 | 0 | 0 | 0 | 0 | −23.55 | 0.53 | 0.92 |
| O43719 | HTATSF1 | 4\|2\|3 | 9 | 3 | 3\|3\|5 | 0 | 0 | 0 | 0 | 0 | −13.04 | 0.82 | 0.88 |
| P35637-2 | FUS | 3\|6\|7 | 16 | 5.33 | 6\|11\|11 | 0 | 0 | 0 | 0 | 0 | −33.63 | 0.57 | 0.92 |
| P27816-4 | MAP4 | 9\|9\|8 | 26 | 8.67 | 23\|14\|15 | 0 | 0 | 0 | 0 | 0 | −54.87 | 0.5 | 0.92 |
| Q14687-2 | GSE1 | 6\|7\|9 | 22 | 7.33 | 6\|5\|7 | 0 | 0 | 0 | 0 | 0 | −15.26 | 1.22 | 0.89 |
| P52272-2 | HNRNPM | 6\|3\|4 | 13 | 4.33 | 8\|8\|12 | 0 | 0 | 0 | 0 | 0 | −33.63 | 0.46 | 0.92 |
| Q9UHI6 | DDX20 | 0\|4\|5 | 9 | 3 | 3\|2\|3 | 0 | 0.01 | 0 | 0.01 | 0 | −13.44 | 1.12 | 0.83 |
| P46977 | STT3A | 4\|3\|3 | 10 | 3.33 | 0\|4\|3 | 0 | 0.01 | 0 | 0.01 | 0 | −6.46 | 1.43 | 0.81 |
| Q9H9B4 | SFXN1 | 4\|5\|8 | 17 | 5.67 | 8\|7\|7 | 0 | 0 | 0 | 0 | 0 | −23.44 | 0.77 | 0.9 |
| Q8NDV7-2 | TNRC6A | 9\|18\|12 | 39 | 13 | 8\|6\|12 | 0 | 0 | 0 | 0 | 0 | −20.37 | 1.5 | 0.86 |
| P34932 | HSPA4 | 14\|8\|12 | 34 | 11.33 | 5\|5\|8 | 0 | 0.01 | 0 | 0.01 | 0 | −12.39 | 1.89 | 0.81 |
| P82675 | MRPS5 | 4\|2\|5 | 11 | 3.67 | 4\|3\|4 | 0 | 0 | 0 | 0 | 0 | −13.04 | 1 | 0.87 |
| Q8IXB1-2 | DNAJC10 | 0\|0\|2 | 2 | 0.67 | 3\|4\|3 | 0 | 0 | 0 | 0 | 0 | −16.29 | 0.2 | 0.92 |
| P21291 | CSRP1 | 9\|8\|5 | 22 | 7.33 | 8\|8\|8 | 0 | 0 | 0 | 0 | 0 | −24.14 | 0.92 | 0.9 |
| P26358-2 | DNMT1 | 13\|17\|16 | 46 | 15.33 | 18\|16\|16 | 0 | 0 | 0 | 0 | 0 | −42.99 | 0.92 | 0.91 |
| Q8IWA0 | WDR75 | 5\|2\|3 | 10 | 3.33 | 4\|7\|9 | 0 | 0 | 0 | 0 | 0 | −25.03 | 0.5 | 0.92 |
| O95456-2 | PSMG1 | 4\|5\|6 | 15 | 5 | 6\|6\|8 | 0 | 0 | 0 | 0 | 0 | −20.88 | 0.75 | 0.92 |
| P46779-4 | RPL28 | 12\|13\|20 | 45 | 15 | 11\|10\|12 | 0 | 0 | 0 | 0 | 0 | −24.18 | 1.36 | 0.9 |
| Q04695 | KRT17 | 54\|45\|40 | 139 | 46.33 | 41\|38\|46 | 0 | 0 | 0 | 0 | 0 | −91.66 | 1.11 | 0.92 |
| Q8NI36 | WDR36 | 0\|4\|5 | 9 | 3 | 7\|5\|12 | 0 | 0 | 0 | 0 | 0 | −36.69 | 0.38 | 0.92 |
| Q71RC2-3 | LARP4 | 11\|12\|14 | 37 | 12.33 | 17\|13\|8 | 0 | 0 | 0 | 0 | 0 | −31.43 | 0.97 | 0.91 |
| Q96A08 | HIST1H2BA | 0\|0\|3 | 3 | 1 | 3\|2\|0 | 0 | 0.01 | 0 | 0.01 | 0 | −9.18 | 0.6 | 0.81 |
| Q8NE71-2 | ABCF1 | 7\|5\|7 | 19 | 6.33 | 13\|10\|11 | 0 | 0 | 0 | 0 | 0 | −37.26 | 0.56 | 0.92 |
| P31930 | UQCRC1 | 8\|6\|7 | 21 | 7 | 14\|15\|12 | 0 | 0 | 0 | 0 | 0 | −44.49 | 0.51 | 0.92 |
| P04844 | RPN2 | 11\|10\|9 | 30 | 10 | 11\|8\|14 | 0 | 0 | 0 | 0 | 0 | −28.61 | 0.91 | 0.92 |
| P04843 | RPN1 | 14\|15\|16 | 45 | 15 | 14\|15\|15 | 0 | 0 | 0 | 0 | 0 | −34.2 | 1.02 | 0.91 |
| P55265 | ADAR | 12\|13\|14 | 39 | 13 | 18\|23\|17 | 0 | 0 | 0 | 0 | 0 | −54.84 | 0.67 | 0.92 |
| Q15366-6 | PCBP2 | 27\|20\|32 | 79 | 26.33 | 22\|25\|27 | 0 | 0 | 0 | 0 | 0 | −61.08 | 1.07 | 0.92 |
| O95235-2 | KIF20A | 0\|3\|4 | 7 | 2.33 | 4\|8\|6 | 0 | 0 | 0 | 0 | 0 | −27.96 | 0.39 | 0.92 |
| P61221 | ABCE1 | 5\|5\|4 | 14 | 4.67 | 6\|5\|5 | 0 | 0 | 0 | 0 | 0 | −15.87 | 0.88 | 0.92 |
| P61254 | RPL26 | 16\|11\|12 | 39 | 13 | 16\|11\|13 | 0 | 0 | 0 | 0 | 0 | −33.97 | 0.97 | 0.91 |
| P27694 | RPA1 | 21\|9\|14 | 44 | 14.67 | 12\|10\|19 | 0 | 0 | 0 | 0 | 0 | −38.46 | 1.07 | 0.91 |
| Q15459 | SF3A1 | 42\|36\|37 | 115 | 38.33 | 44\|37\|47 | 0 | 0 | 0 | 0 | 0 | −101.4 | 0.9 | 0.92 |
| Q96EY1-2 | DNAJA3 | 3\|3\|4 | 10 | 3.33 | 5\|7\|5 | 0 | 0 | 0 | 0 | 0 | −18.88 | 0.59 | 0.92 |
| P55072 | VCP | 10\|7\|9 | 26 | 8.67 | 8\|4\|4 | 0 | 0 | 0 | 0 | 0 | −11.48 | 1.62 | 0.86 |
| P51571 | SSR4 | 4\|4\|3 | 11 | 3.67 | 3\|4\|3 | 0 | 0 | 0 | 0 | 0 | −10.22 | 1.1 | 0.87 |
| P51572 | BCAP31 | 3\|5\|6 | 14 | 4.67 | 5\|7\|7 | 0 | 0 | 0 | 0 | 0 | −21.48 | 0.74 | 0.92 |
| P06737-2 | PYGL | 0\|0\|3 | 3 | 1 | 6\|7\|5 | 0 | 0 | 0 | 0 | 0 | −27.96 | 0.17 | 0.92 |
| P13861-2 | PRKAR2A | 2\|4\|2 | 8 | 2.67 | 3\|6\|7 | 0 | 0 | 0 | 0 | 0 | −19.58 | 0.5 | 0.92 |
| Q9BYD1 | MRPL13 | 3\|3\|0 | 6 | 2 | 2\|3\|3 | 0 | 0 | 0 | 0 | 0 | −13.44 | 0.75 | 0.86 |
| P13645 | KRT10 | 43\|41\|20 | 104 | 34.67 | 38\|58\|36 | 0 | 0 | 0 | 0 | 0 | −135.86 | 0.79 | 0.92 |
| P13647 | KRT5 | 32\|12\|8 | 52 | 17.33 | 18\|17\|11 | 0 | 0 | 0 | 0 | 0 | −46.96 | 1.13 | 0.89 |
| Q9BTE3-2 | MCMBP | 4\|3\|5 | 12 | 4 | 6\|6\|4 | 0 | 0 | 0 | 0 | 0 | −17.58 | 0.75 | 0.92 |
| Q07020 | RPL18 | 12\|11\|12 | 35 | 11.67 | 14\|14\|14 | 0 | 0 | 0 | 0 | 0 | −36.4 | 0.83 | 0.92 |
| Q9NSE4 | IARS2 | 16\|13\|15 | 44 | 14.67 | 14\|18\|15 | 0 | 0 | 0 | 0 | 0 | −39.32 | 0.94 | 0.91 |
| Q9NYF8-3 | BCLAF1 | 2\|3\|6 | 11 | 3.67 | 4\|2\|4 | 0 | 0 | 0 | 0 | 0 | −11.76 | 1.1 | 0.85 |
| Q03252 | LMNB2 | 0\|0\|4 | 4 | 1.33 | 3\|5\|3 | 0 | 0 | 0 | 0 | 0 | −17.76 | 0.36 | 0.88 |
| P17612 | PRKACA | 3\|3\|3 | 9 | 3 | 6\|4\|4 | 0 | 0 | 0 | 0 | 0 | −15.06 | 0.64 | 0.92 |
| Q5SW79 | CEP170 | 6\|5\|4 | 15 | 5 | 7\|6\|10 | 0 | 0 | 0 | 0 | 0 | −24.75 | 0.65 | 0.92 |
| Q04917 | YWHAH | 5\|4\|3 | 12 | 4 | 4\|5\|9 | 0 | 0 | 0 | 0 | 0 | −20.17 | 0.67 | 0.92 |
| Q9Y230 | RUVBL2 | 16\|15\|12 | 43 | 14.33 | 17\|15\|18 | 0 | 0 | 0 | 0 | 0 | −44.68 | 0.86 | 0.92 |
| Q92974-3 | ARHGEF2 | 4\|6\|7 | 17 | 5.67 | 8\|11\|8 | 0 | 0 | 0 | 0 | 0 | −30.01 | 0.63 | 0.92 |
| P10515 | DLAT | 13\|15\|13 | 41 | 13.67 | 13\|13\|13 | 0 | 0 | 0 | 0 | 0 | −29.78 | 1.05 | 0.91 |
| Q9Y606-2 | PUS1 | 4\|3\|2 | 9 | 3 | 0\|5\|3 | 0 | 0 | 0 | 0 | 0 | −9.12 | 1.12 | 0.84 |
| P98170 | XIAP | 18\|17\|23 | 58 | 19.33 | 19\|18\|15 | 0 | 0 | 0 | 0 | 0 | −39.25 | 1.12 | 0.91 |
| P08237 | PFKM | 20\|18\|17 | 55 | 18.33 | 18\|18\|17 | 0 | 0 | 0 | 0 | 0 | −40.42 | 1.04 | 0.91 |
| P18754 | RCC1 | 10\|9\|9 | 28 | 9.33 | 8\|8\|8 | 0 | 0 | 0 | 0 | 0 | −17.96 | 1.17 | 0.9 |
| P08238 | HSP90AB1 | 98\|90\|94 | 282 | 94 | 111\|111\| | 0 | 0 | 0 | 0 | 0 | −262.89 | 0.85 | 0.92 |
| | | | | | 109 | | | | | | | | |
| Q7L014 | DDX46 | 0\|2\|4 | 6 | 2 | 16\|14\|8 | 0 | 0 | 0 | 0 | 0 | −57.57 | 0.16 | 0.92 |
| Q6P1J9 | CDC73 | 3\|0\|2 | 5 | 1.67 | 2\|4\|4 | 0 | 0 | 0 | 0 | 0 | −16.29 | 0.5 | 0.92 |
| Q1KMD3 | HNRNPUL2 | 2\|0\|0 | 2 | 0.67 | 5\|5\|3 | 0 | 0 | 0 | 0 | 0 | −20.66 | 0.15 | 0.92 |
| O15226 | NKRF | 2\|0\|0 | 2 | 0.67 | 5\|3\|0 | 0 | 0 | 0 | 0 | 0 | −13.21 | 0.25 | 0.92 |
| Q9P2J5 | LARS | 12\|9\|9 | 30 | 10 | 17\|11\|19 | 0 | 0 | 0 | 0 | 0 | −46.36 | 0.64 | 0.92 |
| P28340 | POLD1 | 2\|5\|3 | 10 | 3.33 | 3\|4\|3 | 0 | 0 | 0 | 0 | 0 | −11.76 | 1 | 0.86 |
| P35232 | PHB | 3\|3\|2 | 8 | 2.67 | 5\|3\|2 | 0 | 0 | 0 | 0 | 0 | −11.76 | 0.8 | 0.92 |
| P30876 | POLR2B | 9\|5\|4 | 18 | 6 | 4\|6\|6 | 0 | 0 | 0 | 0 | 0 | −15.87 | 1.12 | 0.87 |
| P15924 | DSP | 42\|20\|24 | 86 | 28.67 | 28\|30\|26 | 0 | 0 | 0 | 0 | 0 | −73.52 | 1.02 | 0.92 |
| P52948-6 | NUP98 | 26\|27\|25 | 78 | 26 | 19\|22\|25 | 0 | 0 | 0 | 0 | 0 | −44.12 | 1.18 | 0.92 |
| P36542 | ATP5C1 | 7\|7\|5 | 19 | 6.33 | 7\|8\|12 | 0 | 0 | 0 | 0 | 0 | −28 | 0.7 | 0.92 |
| P40616-2 | ARL1 | 3\|3\|3 | 9 | 3 | 2\|5\|5 | 0 | 0 | 0 | 0 | 0 | −12.55 | 0.75 | 0.92 |
| Q9Y3F4 | STRAP | 6\|4\|8 | 18 | 6 | 6\|7\|6 | 0 | 0 | 0 | 0 | 0 | −19.6 | 0.95 | 0.9 |
| P17980 | PSMC3 | 9\|9\|8 | 26 | 8.67 | 8\|9\|6 | 0 | 0 | 0 | 0 | 0 | −18.23 | 1.13 | 0.9 |
| P17987 | TCP1 | 42\|32\|38 | 112 | 37.33 | 44\|41\|48 | 0 | 0 | 0 | 0 | 0 | −114.28 | 0.84 | 0.92 |
| P06239 | LCK | 3\|2\|3 | 8 | 2.67 | 3\|3\|2 | 0 | 0 | 0 | 0 | 0 | −9.34 | 1 | 0.86 |
| P63220 | RPS21 | 4\|6\|4 | 14 | 4.67 | 3\|4\|4 | 0 | 0 | 0 | 0 | 0 | −9.93 | 1.27 | 0.86 |
| O43143 | DHX15 | 14\|17\|19 | 50 | 16.67 | 14\|21\|19 | 0 | 0 | 0 | 0 | 0 | −46.27 | 0.93 | 0.91 |
| P67775 | PPP2CA | 3\|4\|6 | 13 | 4.33 | 5\|7\|6 | 0 | 0 | 0 | 0 | 0 | −20.15 | 0.72 | 0.92 |
| P28072 | PSMB6 | 0\|2\|2 | 4 | 1.33 | 3\|3\|2 | 0 | 0 | 0 | 0 | 0 | −13.44 | 0.5 | 0.92 |
| O75663 | TIPRL | 4\|2\|0 | 6 | 2 | 3\|0\|4 | 0 | 0.01 | 0 | 0.01 | 0 | −11.81 | 0.86 | 0.83 |
| Q8IYB8 | SUPV3L1 | 2\|0\|0 | 2 | 0.67 | 3\|0\|2 | 0 | 0 | 0 | 0 | 0 | −9.18 | 0.4 | 0.84 |
| P53597 | SUCLG1 | 0\|3\|2 | 5 | 1.67 | 2\|2\|3 | 0 | 0 | 0 | 0 | 0 | −11.98 | 0.71 | 0.85 |
| Q6PI48 | DARS2 | 6\|5\|6 | 17 | 5.67 | 7\|9\|7 | 0 | 0 | 0 | 0 | 0 | −22.9 | 0.74 | 0.92 |
| O43709 | BUD23 | 4\|4\|5 | 13 | 4.33 | 4\|5\|4 | 0 | 0 | 0 | 0 | 0 | −12.29 | 1 | 0.89 |
| P84103-2 | SRSF3 | 2\|0\|0 | 2 | 0.67 | 2\|0\|5 | 0 | 0 | 0 | 0 | 0 | −11.64 | 0.29 | 0.92 |
| P04406 | GAPDH | 36\|35\|36 | 107 | 35.67 | 37\|36\|30 | 0 | 0 | 0 | 0 | 0 | −73.05 | 1.04 | 0.92 |
| P42285 | SKIV2L2 | 4\|5\|6 | 15 | 5 | 8\|6\|5 | 0 | 0 | 0 | 0 | 0 | −19.6 | 0.79 | 0.92 |
| P62424 | RPL7A | 22\|16\|20 | 58 | 19.33 | 30\|24\|22 | 0 | 0 | 0 | 0 | 0 | −70.54 | 0.76 | 0.92 |
| P28331-3 | NDUFS1 | 5\|2\|4 | 11 | 3.67 | 4\|3\|3 | 0 | 0 | 0 | 0 | 0 | −11.76 | 1.1 | 0.86 |
| P31939-2 | ATIC | 7\|7\|6 | 20 | 6.67 | 7\|7\|4 | 0 | 0 | 0 | 0 | 0 | −15.26 | 1.11 | 0.9 |
| Q14152 | EIF3A | 0\|4\|4 | 8 | 2.67 | 12\|7\|4 | 0 | 0 | 0 | 0 | 0 | −35.13 | 0.35 | 0.92 |
| Q14151 | SAFB2 | 3\|0\|2 | 5 | 1.67 | 13\|10\|13 | 0 | 0 | 0 | 0 | 0 | −54.62 | 0.14 | 0.92 |
| P26038 | MSN | 2\|2\|3 | 7 | 2.33 | 2\|3\|2 | 0 | 0 | 0 | 0 | 0 | −8.15 | 1 | 0.85 |
| Q9Y5J1 | UTP18 | 2\|0\|2 | 4 | 1.33 | 3\|3\|3 | 0 | 0 | 0 | 0 | 0 | −14.8 | 0.44 | 0.92 |
| P19623 | SRM | 5\|4\|7 | 16 | 5.33 | 4\|3\|5 | 0 | 0 | 0 | 0 | 0 | −11.11 | 1.33 | 0.86 |
| P34897-3 | SHMT2 | 5\|9\|7 | 21 | 7 | 5\|7\|7 | 0 | 0 | 0 | 0 | 0 | −17.93 | 1.11 | 0.89 |
| P63241 | EIF5A | 9\|8\|8 | 25 | 8.33 | 8\|6\|6 | 0 | 0 | 0 | 0 | 0 | −14.71 | 1.25 | 0.89 |
| P63244 | RACK1 | 11\|10\|12 | 33 | 11 | 11\|14\|15 | 0 | 0 | 0 | 0 | 0 | −35.6 | 0.82 | 0.92 |
| Q13443 | ADAM9 | 0\|2\|3 | 5 | 1.67 | 5\|2\|3 | 0 | 0 | 0 | 0 | 0 | −16.29 | 0.5 | 0.92 |
| P62333 | PSMC6 | 6\|7\|6 | 19 | 6.33 | 10\|5\|9 | 0 | 0 | 0 | 0 | 0 | −22.42 | 0.79 | 0.92 |
| Q16576 | RBBP7 | 5\|4\|4 | 13 | 4.33 | 8\|7\|6 | 0 | 0 | 0 | 0 | 0 | −22.13 | 0.62 | 0.92 |
| Q9NZB2 | FAM120A | 4\|7\|5 | 16 | 5.33 | 8\|8\|5 | 0 | 0 | 0 | 0 | 0 | −22.13 | 0.76 | 0.92 |
| P30479 | HLA-B | 0\|2\|0 | 2 | 0.67 | 5\|3\|2 | 0 | 0 | 0 | 0 | 0 | −16.29 | 0.2 | 0.92 |
| O60566 | BUB1B | 6\|8\|12 | 26 | 8.67 | 8\|11\|9 | 0 | 0 | 0 | 0 | 0 | −27.45 | 0.93 | 0.9 |
| Q9UBU9 | NXF1 | 6\|6\|9 | 21 | 7 | 8\|6\|10 | 0 | 0 | 0 | 0 | 0 | −22.42 | 0.88 | 0.9 |
| Q13509 | TUBB3 | 59\|57\|64 | 180 | 60 | 62\|61\|59 | 0 | 0 | 0 | 0 | 0 | −134.02 | 0.99 | 0.92 |
| P61026 | RAB10 | 5\|4\|5 | 14 | 4.67 | 9\|9\|12 | 0 | 0 | 0 | 0 | 0 | −34.04 | 0.47 | 0.92 |
| P61020 | RAB5B | 3\|0\|6 | 9 | 3 | 6\|6\|7 | 0 | 0 | 0 | 0 | 0 | −29.46 | 0.47 | 0.92 |
| P59998 | ARPC4 | 2\|3\|3 | 8 | 2.67 | 2\|3\|2 | 0 | 0 | 0 | 0 | 0 | −8.15 | 1.14 | 0.85 |
| P12694 | BCKDHA | 2\|0\|2 | 4 | 1.33 | 3\|3\|2 | 0 | 0 | 0 | 0 | 0 | −13.44 | 0.5 | 0.92 |
| Q16719 | KYNU | 2\|4\|3 | 9 | 3 | 2\|2\|3 | 0 | 0.01 | 0 | 0.01 | 0 | −8.15 | 1.29 | 0.83 |
| Q7L576 | CYFIP1 | 2\|4\|3 | 9 | 3 | 3\|3\|3 | 0 | 0 | 0 | 0 | 0 | −10.47 | 1 | 0.86 |
| Q9UQE7 | SMC3 | 14\|9\|8 | 31 | 10.33 | 15\|12\|14 | 0 | 0 | 0 | 0 | 0 | −40.44 | 0.76 | 0.91 |
| P48634 | PRRC2A | 2\|3\|5 | 10 | 3.33 | 6\|4\|7 | 0 | 0 | 0 | 0 | 0 | −20.94 | 0.59 | 0.92 |
| Q96KR1 | ZFR | 8\|6\|7 | 21 | 7 | 17\|14\|12 | 0 | 0 | 0 | 0 | 0 | −47.18 | 0.49 | 0.92 |
| Q15286 | RAB35 | 4\|5\|4 | 13 | 4.33 | 5\|6\|4 | 0 | 0 | 0 | 0 | 0 | −14.62 | 0.87 | 0.92 |
| P61247 | RPS3A | 25\|14\|16 | 55 | 18.33 | 12\|12\|14 | 0 | 0 | 0 | 0 | 0 | −27.16 | 1.45 | 0.89 |
| Q13619 | CUL4A | 5\|3\|3 | 11 | 3.67 | 9\|10\|11 | 0 | 0 | 0 | 0 | 0 | −36.37 | 0.37 | 0.92 |
| P20339-2 | RAB5A | 4\|4\|6 | 14 | 4.67 | 4\|6\|7 | 0 | 0 | 0 | 0 | 0 | −17.11 | 0.82 | 0.9 |
| P15311 | EZR | 3\|2\|0 | 5 | 1.67 | 3\|5\|4 | 0 | 0 | 0 | 0 | 0 | −19.16 | 0.42 | 0.92 |
| P60866 | RPS20 | 5\|3\|3 | 11 | 3.67 | 6\|4\|3 | 0 | 0 | 0 | 0 | 0 | −13.81 | 0.85 | 0.89 |
| Q9BQC3-2 | DPH2 | 0\|0\|2 | 2 | 0.67 | 2\|0\|3 | 0 | 0 | 0 | 0 | 0 | −9.18 | 0.4 | 0.84 |
| P11908 | PRPS2 | 9\|7\|8 | 24 | 8 | 4\|7\|8 | 0 | 0 | 0 | 0 | 0 | −14.98 | 1.26 | 0.89 |
| P56537 | EIF6 | 9\|6\|6 | 21 | 7 | 6\|6\|6 | 0 | 0 | 0 | 0 | 0 | −15.26 | 1.17 | 0.89 |
| P10909-4 | CLU | 7\|14\|11 | 32 | 10.67 | 10\|8\|7 | 0 | 0 | 0 | 0 | 0 | −22.07 | 1.28 | 0.89 |
| O14776-2 | TCERG1 | 0\|2\|0 | 2 | 0.67 | 2\|3\|0 | 0 | 0 | 0 | 0 | 0 | −9.18 | 0.4 | 0.84 |
| Q6S8J3 | POTEE | 25\|19\|18 | 62 | 20.67 | 22\|24\|19 | 0 | 0 | 0 | 0 | 0 | −53.29 | 0.95 | 0.92 |
| O43390-4 | HNRNPR | 3\|4\|2 | 9 | 3 | 3\|3\|3 | 0 | 0 | 0 | 0 | 0 | −10.47 | 1 | 0.86 |
| P29353 | SHC1 | 7\|4\|6 | 17 | 5.67 | 7\|6\|7 | 0 | 0 | 0 | 0 | 0 | −20.88 | 0.85 | 0.9 |
| Q12904 | AIMP1 | 6\|3\|3 | 12 | 4 | 5\|3\|5 | 0 | 0 | 0 | 0 | 0 | −13.81 | 0.92 | 0.88 |
| P49419-2 | ALDH7A1 | 8\|3\|9 | 20 | 6.67 | 7\|3\|5 | 0 | 0 | 0 | 0 | 0 | −16.28 | 1.33 | 0.86 |
| Q9UJW0 | DCTN4 | 2\|0\|2 | 4 | 1.33 | 4\|4\|5 | 0 | 0 | 0 | 0 | 0 | −20.66 | 0.31 | 0.92 |
| Q9BXY0 | MAK16 | 0\|2\|2 | 4 | 1.33 | 2\|2\|2 | 0 | 0 | 0 | 0 | 0 | −10.49 | 0.67 | 0.92 |
| Q9P035 | HACD3 | 0\|0\|2 | 2 | 0.67 | 3\|3\|3 | 0 | 0 | 0 | 0 | 0 | −14.8 | 0.22 | 0.92 |
| Q00341-2 | HDLBP | 10\|7\|9 | 26 | 8.67 | 7\|5\|6 | 0 | 0 | 0 | 0 | 0 | −13.81 | 1.44 | 0.88 |
| P27816 | MAP4 | 17\|16\|18 | 51 | 17 | 36\|33\|26 | 0 | 0 | 0 | 0 | 0 | −95.41 | 0.54 | 0.92 |
| Q12874 | SF3A3 | 16\|16\|19 | 51 | 17 | 15\|13\|16 | 0 | 0 | 0 | 0 | 0 | −31.3 | 1.16 | 0.91 |
| Q13363-2 | CTBP1 | 3\|0\|0 | 3 | 1 | 4\|2\|3 | 0 | 0 | 0 | 0 | 0 | −14.8 | 0.33 | 0.92 |
| P51532-5 | SMARCA4 | 2\|0\|3 | 5 | 1.67 | 3\|4\|2 | 0 | 0 | 0 | 0 | 0 | −14.8 | 0.56 | 0.92 |
| Q93052 | LPP | 5\|7\|6 | 18 | 6 | 14\|8\|9 | 0 | 0 | 0 | 0 | 0 | −33.26 | 0.58 | 0.92 |
| P08754 | GNAI3 | 3\|4\|5 | 12 | 4 | 6\|6\|7 | 0 | 0 | 0 | 0 | 0 | −21.48 | 0.63 | 0.92 |
| P31327 | CPS1 | 112\|102\|101 | 315 | 105 | 131\|130\| | 0 | 0 | 0 | 0 | 0 | −313.92 | 0.81 | 0.92 |
| | | | | | 126 | | | | | | | | |
| P62937 | PPIA | 11\|12\|16 | 39 | 13 | 13\|16\|15 | 0 | 0 | 0 | 0 | 0 | −38.89 | 0.89 | 0.91 |
| P09622 | DLD | 13\|9\|8 | 30 | 10 | 10\|8\|9 | 0 | 0 | 0 | 0 | 0 | −22.92 | 1.11 | 0.9 |
| Q00325-2 | SLC25A3 | 12\|10\|13 | 35 | 11.67 | 14\|13\|13 | 0 | 0 | 0 | 0 | 0 | −35.6 | 0.87 | 0.92 |
| O43865 | AHCYL1 | 5\|8\|7 | 20 | 6.67 | 7\|8\|11 | 0 | 0 | 0 | 0 | 0 | −26.73 | 0.77 | 0.92 |
| P36776-2 | LONP1 | 3\|3\|5 | 11 | 3.67 | 6\|8\|11 | 0 | 0 | 0 | 0 | 0 | −29.52 | 0.44 | 0.92 |
| Q92905 | COPS5 | 4\|3\|4 | 11 | 3.67 | 4\|2\|3 | 0 | 0 | 0 | 0 | 0 | −9.04 | 1.22 | 0.86 |
| Q9Y314 | NOSIP | 8\|7\|6 | 21 | 7 | 6\|6\|5 | 0 | 0 | 0 | 0 | 0 | −14.08 | 1.24 | 0.89 |
| P46776 | RPL27A | 9\|9\|6 | 24 | 8 | 10\|5\|9 | 0 | 0 | 0 | 0 | 0 | −22.42 | 1 | 0.9 |
| P46778 | RPL21 | 23\|27\|27 | 77 | 25.67 | 29\|24\|21 | 0 | 0 | 0 | 0 | 0 | −56.41 | 1.04 | 0.92 |
| O60701-2 | UGDH | 7\|6\|8 | 21 | 7 | 6\|6\|7 | 0 | 0 | 0 | 0 | 0 | −16.44 | 1.11 | 0.9 |
| P43897 | TSFM | 4\|0\|3 | 7 | 2.33 | 4\|3\|7 | 0 | 0 | 0 | 0 | 0 | −22.13 | 0.5 | 0.92 |
| Q3ZCQ8 | TIMM50 | 0\|3\|3 | 6 | 2 | 4\|6\|5 | 0 | 0 | 0 | 0 | 0 | −23.55 | 0.4 | 0.92 |
| P34896-2 | SHMT1 | 2\|3\|2 | 7 | 2.33 | 3\|4\|4 | 0 | 0 | 0 | 0 | 0 | −13.04 | 0.64 | 0.92 |
| Q6P2Q9 | PRPF8 | 18\|13\|17 | 48 | 16 | 30\|35\|35 | 0 | 0 | 0 | 0 | 0 | −108.73 | 0.48 | 0.92 |
| O75874 | IDH1 | 14\|13\|18 | 45 | 15 | 19\|14\|16 | 0 | 0 | 0 | 0 | 0 | −41.76 | 0.92 | 0.91 |
| Q92878 | RAD50 | 3\|0\|2 | 5 | 1.67 | 11\|7\|5 | 0 | 0 | 0 | 0 | 0 | −35.38 | 0.22 | 0.92 |
| P06241 | FYN | 6\|4\|4 | 14 | 4.67 | 5\|5\|4 | 0 | 0 | 0 | 0 | 0 | −13.48 | 1 | 0.89 |
| Q5T4S7-3 | UBR4 | 6\|3\|5 | 14 | 4.67 | 5\|0\|4 | 0 | 0.01 | 0 | 0.01 | 0 | −8.72 | 1.56 | 0.81 |
| Q9Y3U8 | RPL36 | 3\|4\|3 | 10 | 3.33 | 2\|3\|4 | 0 | 0 | 0 | 0 | 0 | −9.04 | 1.11 | 0.86 |
| Q68CZ2 | TNS3 | 0\|4\|2 | 6 | 2 | 3\|4\|7 | 0 | 0 | 0 | 0 | 0 | −22.13 | 0.43 | 0.92 |
| P63010-3 | AP2B1 | 4\|4\|7 | 15 | 5 | 5\|4\|4 | 0 | 0 | 0 | 0 | 0 | −12.29 | 1.15 | 0.87 |
| Q5HY18 | RABL3 | 2\|0\|0 | 2 | 0.67 | 0\|6\|7 | 0 | 0 | 0 | 0 | 0 | −20.2 | 0.15 | 0.92 |
| P62753 | RPS6 | 13\|13\|19 | 45 | 15 | 19\|16\|20 | 0 | 0 | 0 | 0 | 0 | −49.22 | 0.82 | 0.91 |
| P62750 | RPL23A | 7\|8\|7 | 22 | 7.33 | 11\|8\|9 | 0 | 0 | 0 | 0 | 0 | −25.73 | 0.79 | 0.92 |
| P22392 | NME2 | 0\|2\|0 | 2 | 0.67 | 3\|2\|2 | 0 | 0 | 0 | 0 | 0 | −11.98 | 0.29 | 0.92 |
| P04075 | ALDOA | 10\|14\|13 | 37 | 12.33 | 13\|13\|16 | 0 | 0 | 0 | 0 | 0 | −38.08 | 0.88 | 0.92 |
| P09104-2 | ENO2 | 3\|2\|3 | 8 | 2.67 | 0\|3\|5 | 0 | 0 | 0 | 0 | 0 | −9.12 | 1 | 0.86 |
| Q6UB35 | MTHFD1L | 5\|3\|5 | 13 | 4.33 | 5\|7\|6 | 0 | 0 | 0 | 0 | 0 | −20.15 | 0.72 | 0.92 |
| P41250 | GARS | 21\|20\|24 | 65 | 21.67 | 24\|29\|33 | 0 | 0 | 0 | 0 | 0 | −76.05 | 0.76 | 0.92 |
| P41252 | IARS | 10\|5\|9 | 24 | 8 | 12\|11\|15 | 0 | 0 | 0 | 0 | 0 | −42.65 | 0.63 | 0.92 |
| Q9Y5Y2 | NUBP2 | 0\|2\|3 | 5 | 1.67 | 6\|0\|5 | 0 | 0 | 0 | 0 | 0 | −17.44 | 0.45 | 0.92 |
| Q14697-2 | GANAB | 13\|17\|14 | 44 | 14.67 | 17\|17\|16 | 0 | 0 | 0 | 0 | 0 | −42.99 | 0.88 | 0.91 |
| Q7KZI7-15 | MARK2 | 9\|4\|3 | 16 | 5.33 | 4\|3\|5 | 0 | 0.01 | 0 | 0.01 | 0 | −12.55 | 1.33 | 0.81 |
| Q13813-3 | SPTAN1 | 3\|3\|2 | 8 | 2.67 | 10\|11\|12 | 0 | 0 | 0 | 0 | 0 | −43.25 | 0.24 | 0.92 |
| Q9H4B7 | TUBB1 | 11\|12\|12 | 35 | 11.67 | 13\|11\|10 | 0 | 0 | 0 | 0 | 0 | −26.81 | 1.03 | 0.91 |
| P15121 | AKR1B1 | 2\|6\|6 | 14 | 4.67 | 4\|3\|3 | 0 | 0 | 0 | 0 | 0 | −11.76 | 1.4 | 0.83 |
| Q8TCU4-3 | ALMS1 | 0\|2\|2 | 4 | 1.33 | 2\|3\|2 | 0 | 0 | 0 | 0 | 0 | −11.98 | 0.57 | 0.92 |
| P46821 | MAP1B | 4\|5\|6 | 15 | 5 | 7\|4\|9 | 0 | 0 | 0 | 0 | 0 | −20.88 | 0.75 | 0.92 |
| P09543-2 | CNP | 6\|7\|8 | 21 | 7 | 5\|4\|6 | 0 | 0 | 0 | 0 | 0 | −11.74 | 1.4 | 0.87 |
| O60341 | KDM1A | 3\|2\|3 | 8 | 2.67 | 4\|4\|2 | 0 | 0 | 0 | 0 | 0 | −11.76 | 0.8 | 0.92 |
| Q6ZSR9 | Q6ZSR9 | 0\|3\|2 | 5 | 1.67 | 2\|2\|2 | 0 | 0 | 0 | 0 | 0 | −10.49 | 0.83 | 0.84 |
| Q9NV06 | DCAF13 | 2\|2\|2 | 6 | 2 | 4\|4\|0 | 0 | 0 | 0 | 0 | 0 | −9.18 | 0.75 | 0.92 |
| P19367-4 | HK1 | 3\|3\|4 | 10 | 3.33 | 2\|2\|3 | 0 | 0.01 | 0 | 0.01 | 0 | −6.68 | 1.43 | 0.83 |
| P61019 | RAB2A | 11\|8\|7 | 26 | 8.67 | 4\|7\|7 | 0 | 0 | 0 | 0 | 0 | −13.81 | 1.44 | 0.87 |
| Q6DKI1 | RPL7L1 | 2\|0\|2 | 4 | 1.33 | 3\|2\|3 | 0 | 0 | 0 | 0 | 0 | −13.44 | 0.5 | 0.92 |
| Q9NW82 | WDR70 | 4\|2\|2 | 8 | 2.67 | 4\|2\|3 | 0 | 0 | 0 | 0 | 0 | −10.47 | 0.89 | 0.86 |
| P07910-2 | HNRNPC | 4\|7\|7 | 18 | 6 | 12\|13\|8 | 0 | 0 | 0 | 0 | 0 | −38.11 | 0.55 | 0.92 |
| Q13618-2 | CUL3 | 4\|4\|4 | 12 | 4 | 6\|7\|4 | 0 | 0 | 0 | 0 | 0 | −17.11 | 0.71 | 0.92 |
| Q8IX12-2 | CCAR1 | 2\|0\|0 | 2 | 0.67 | 3\|0\|4 | 0 | 0 | 0 | 0 | 0 | −11.81 | 0.29 | 0.92 |
| Q9Y5K6 | CD2AP | 0\|3\|0 | 3 | 1 | 2\|3\|0 | 0 | 0.01 | 0 | 0.01 | 0 | −9.18 | 0.6 | 0.81 |
| O95433 | AHSA1 | 21\|22\|25 | 68 | 22.67 | 23\|19\|22 | 0 | 0 | 0 | 0 | 0 | −47.54 | 1.06 | 0.92 |
| Q66PJ3-7 | ARL6IP4 | 3\|3\|4 | 10 | 3.33 | 2\|2\|3 | 0 | 0.01 | 0 | 0.01 | 0 | −6.68 | 1.43 | 0.83 |
| Q9NR30 | DDX21 | 19\|19\|25 | 63 | 21 | 33\|30\|35 | 0 | 0 | 0 | 0 | 0 | −93.33 | 0.64 | 0.92 |
| P46087 | NOP2 | 2\|2\|0 | 4 | 1.33 | 6\|4\|3 | 0 | 0 | 0 | 0 | 0 | −20.66 | 0.31 | 0.92 |
| P49736 | MCM2 | 10\|12\|15 | 37 | 12.33 | 10\|12\|12 | 0 | 0 | 0 | 0 | 0 | −28.28 | 1.09 | 0.91 |
| Q96L92 | SNX27 | 2\|2\|2 | 6 | 2 | 4\|2\|4 | 0 | 0 | 0 | 0 | 0 | −11.76 | 0.6 | 0.92 |
| Q9Y2L1 | DIS3 | 5\|6\|7 | 18 | 6 | 14\|10\|8 | 0 | 0 | 0 | 0 | 0 | −34.59 | 0.56 | 0.92 |
| Q9H0H5 | RACGAP1 | 2\|0\|0 | 2 | 0.67 | 2\|0\|3 | 0 | 0 | 0 | 0 | 0 | −9.18 | 0.4 | 0.84 |
| Q15436 | SEC23A | 3\|6\|6 | 15 | 5 | 4\|4\|6 | 0 | 0 | 0 | 0 | 0 | −15.06 | 1.07 | 0.88 |
| P09960 | LTA4H | 0\|0\|2 | 2 | 0.67 | 3\|2\|4 | 0 | 0 | 0 | 0 | 0 | −14.8 | 0.22 | 0.92 |
| P30084 | ECHS1 | 2\|0\|0 | 2 | 0.67 | 2\|2\|2 | 0 | 0 | 0 | 0 | 0 | −10.49 | 0.33 | 0.92 |
| O15027 | SEC16A | 13\|10\|14 | 37 | 12.33 | 20\|19\|22 | 0 | 0 | 0 | 0 | 0 | −62.71 | 0.61 | 0.92 |
| Q0VDF9 | HSPA14 | 2\|0\|3 | 5 | 1.67 | 3\|5\|3 | 0 | 0 | 0 | 0 | 0 | −17.76 | 0.45 | 0.92 |
| O75746-2 | SLC25A12 | 3\|6\|3 | 12 | 4 | 6\|3\|5 | 0 | 0 | 0 | 0 | 0 | −15.06 | 0.86 | 0.89 |
| Q9NQW7 | XPNPEP1 | 2\|0\|0 | 2 | 0.67 | 4\|4\|0 | 0 | 0 | 0 | 0 | 0 | −13.27 | 0.25 | 0.92 |
| Q6P587 | FAHD1 | 3\|2\|3 | 8 | 2.67 | 3\|2\|3 | 0 | 0 | 0 | 0 | 0 | −9.34 | 1 | 0.86 |
| P23921 | RRM1 | 17\|12\|13 | 42 | 14 | 14\|16\|15 | 0 | 0 | 0 | 0 | 0 | −38.46 | 0.93 | 0.91 |
| O00299 | CLIC1 | 8\|9\|10 | 27 | 9 | 9\|7\|7 | 0 | 0 | 0 | 0 | 0 | −18.23 | 1.17 | 0.9 |
| O94925-3 | GLS | 2\|0\|0 | 2 | 0.67 | 3\|3\|6 | 0 | 0 | 0 | 0 | 0 | −19.16 | 0.17 | 0.92 |
| P31689 | DNAJA1 | 5\|8\|5 | 18 | 6 | 5\|4\|5 | 0 | 0 | 0 | 0 | 0 | −12.01 | 1.29 | 0.87 |
| P11802 | CDK4 | 3\|0\|3 | 6 | 2 | 3\|3\|3 | 0 | 0 | 0 | 0 | 0 | −14.8 | 0.67 | 0.92 |
| Q15046 | KARS | 6\|4\|0 | 10 | 3.33 | 9\|6\|8 | 0 | 0 | 0 | 0 | 0 | −35.37 | 0.43 | 0.92 |
| Q12789-3 | GTF3C1 | 0\|0\|3 | 3 | 1 | 3\|3\|2 | 0 | 0 | 0 | 0 | 0 | −13.44 | 0.38 | 0.87 |
| P49915 | GMPS | 15\|13\|16 | 44 | 14.67 | 13\|15\|18 | 0 | 0 | 0 | 0 | 0 | −38.1 | 0.96 | 0.91 |
| P02533 | KRT14 | 51\|24\|16 | 91 | 30.33 | 25\|27\|26 | 0 | 0 | 0 | 0 | 0 | −73.11 | 1.17 | 0.91 |
| Q8IWS0-5 | PHF6 | 2\|0\|7 | 9 | 3 | 4\|7\|5 | 0 | 0 | 0 | 0 | 0 | −25.05 | 0.56 | 0.89 |
| P04264 | KRT1 | 59\|46\|23 | 128 | 42.67 | 65\|62\|40 | 0 | 0 | 0 | 0 | 0 | −176.54 | 0.77 | 0.92 |
| Q9UJU6 | DBNL | 5\|4\|6 | 15 | 5 | 4\|4\|5 | 0 | 0 | 0 | 0 | 0 | −12.29 | 1.15 | 0.88 |
| Q01780 | EXOSC10 | 8\|7\|5 | 20 | 6.67 | 7\|9\|14 | 0 | 0 | 0 | 0 | 0 | −31.9 | 0.67 | 0.92 |
| O60502 | MGEA5 | 0\|7\|3 | 10 | 3.33 | 6\|2\|7 | 0 | 0 | 0 | 0 | 0 | −23.49 | 0.67 | 0.89 |
| Q99426 | TBCB | 9\|5\|9 | 23 | 7.67 | 9\|8\|7 | 0 | 0 | 0 | 0 | 0 | −24.14 | 0.96 | 0.9 |
| P05198 | EIF2S1 | 10\|13\|12 | 35 | 11.67 | 18\|14\|15 | 0 | 0 | 0 | 0 | 0 | −44.43 | 0.74 | 0.92 |
| P07437 | TUBB | 100\|104\|105 | 309 | 103 | 115\|113\| | 0 | 0 | 0 | 0 | 0 | −261.52 | 0.9 | 0.92 |
| | | | | | 115 | | | | | | | | |
| Q9Y490 | TLN1 | 5\|0\|6 | 11 | 3.67 | 6\|6\|5 | 0 | 0 | 0 | 0 | 0 | −26.53 | 0.65 | 0.9 |
| P78347-2 | GTF21 | 2\|2\|4 | 8 | 2.67 | 5\|12\|8 | 0 | 0 | 0 | 0 | 0 | −31.87 | 0.32 | 0.92 |
| Q01650 | SLC7A5 | 5\|5\|5 | 15 | 5 | 854 | 0 | 0 | 0 | 0 | 0 | −15.55 | 0.88 | 0.92 |
| P43243 | MATR3 | 2\|5\|4 | 11 | 3.67 | 10\|7\|10 | 0 | 0 | 0 | 0 | 0 | −34.75 | 0.41 | 0.92 |
| P23381 | WARS | 17\|13\|15 | 45 | 15 | 19\|18\|18 | 0 | 0 | 0 | 0 | 0 | −49.22 | 0.82 | 0.92 |
| P49005 | POLD2 | 7\|4\|5 | 16 | 5.33 | 5\|4\|2 | 0 | 0 | 0 | 0 | 0 | −9.93 | 1.45 | 0.84 |
| Q9NVI7-2 | ATAD3A | 4\|6\|5 | 15 | 5 | 7\|7\|8 | 0 | 0 | 0 | 0 | 0 | −23.44 | 0.68 | 0.92 |
| P00374-2 | DHFR | 3\|3\|0 | 6 | 2 | 2\|3\|3 | 0 | 0 | 0 | 0 | 0 | −13.44 | 0.75 | 0.86 |
| P27708 | CAD | 11\|14\|16 | 41 | 13.67 | 18\|22\|17 | 0 | 0 | 0 | 0 | 0 | −55.44 | 0.72 | 0.92 |
| P31946-2 | YWHAB | 9\|9\|11 | 29 | 9.67 | 8\|13\|14 | 0 | 0 | 0 | 0 | 0 | −31.08 | 0.83 | 0.92 |
| Q9Y4L1 | HYOU1 | 4\|3\|7 | 14 | 4.67 | 6\|6\|9 | 0 | 0 | 0 | 0 | 0 | −24.11 | 0.67 | 0.92 |
| Q9NQW6-2 | ANLN | 3\|3\|7 | 13 | 4.33 | 5\|4\|3 | 0 | 0 | 0 | 0 | 0 | −12.55 | 1.08 | 0.86 |
| Q13283 | G3BP1 | 2\|0\|3 | 5 | 1.67 | 2\|3\|2 | 0 | 0 | 0 | 0 | 0 | −11.98 | 0.71 | 0.85 |
| O75600 | GCAT | 2\|0\|0 | 2 | 0.67 | 0\|2\|2 | 0 | 0.01 | 0 | 0.01 | 0 | −7.85 | 0.5 | 0.82 |
| P23258 | TUBG1 | 7\|3\|3 | 13 | 4.33 | 6\|4\|5 | 0 | 0 | 0 | 0 | 0 | −16.28 | 0.87 | 0.89 |
| Q14103-3 | HNRNPD | 12\|17\|18 | 47 | 15.67 | 24\|18\|20 | 0 | 0 | 0 | 0 | 0 | −60.02 | 0.76 | 0.92 |
| P20042 | EIF2S2 | 11\|14\|12 | 37 | 12.33 | 15\|14\|10 | 0 | 0 | 0 | 0 | 0 | −32.74 | 0.95 | 0.91 |
| Q9Y281-3 | CFL2 | 8\|7\|9 | 24 | 8 | 5\|6\|8 | 0 | 0 | 0 | 0 | 0 | −14.98 | 1.26 | 0.89 |
| Q9GZZ1 | NAA50 | 5\|0\|4 | 9 | 3 | 4\|4\|5 | 0 | 0 | 0 | 0 | 0 | −20.66 | 0.69 | 0.89 |
| O96008 | TOMM40 | 0\|2\|2 | 4 | 1.33 | 2\|6\|0 | 0 | 0 | 0 | 0 | 0 | −13.06 | 0.5 | 0.92 |
| Q99707 | MTR | 4\|5\|6 | 15 | 5 | 5\|6\|4 | 0 | 0 | 0 | 0 | 0 | −14.62 | 1 | 0.89 |
| O94966-2 | USP19 | 0\|0\|2 | 2 | 0.67 | 4\|2\|0 | 0 | 0 | 0 | 0 | 0 | −10.24 | 0.33 | 0.92 |
| P25705 | ATP5A1 | 35\|27\|32 | 94 | 31.33 | 35\|37\|41 | 0 | 0 | 0 | 0 | 0 | −97.82 | 0.83 | 0.92 |
| O43615 | TIMM44 | 6\|4\|5 | 15 | 5 | 7\|6\|6 | 0 | 0 | 0 | 0 | 0 | −19.6 | 0.79 | 0.92 |
| Q8WWM7-6 | ATXN2L | 6\|6\|8 | 20 | 6.67 | 6\|5\|8 | 0 | 0 | 0 | 0 | 0 | −16.44 | 1.05 | 0.9 |
| P61313 | RPL15 | 11\|8\|9 | 28 | 9.33 | 13\|10\|10 | 0 | 0 | 0 | 0 | 0 | −30.24 | 0.85 | 0.92 |
| Q9HCM7 | FBRSL1 | 3\|4\|5 | 12 | 4 | 2\|4\|4 | 0 | 0 | 0 | 0 | 0 | −10.22 | 1.2 | 0.86 |
| P04908 | HIST1H2AE; | 0\|3\|4 | 7 | 2.33 | 8\|6\|8 | 0 | 0 | 0 | 0 | 0 | −33.89 | 0.32 | 0.92 |
| | HIST1H2AB | | | | | | | | | | | | |
| Q13263 | TRIM28 | 4\|3\|4 | 11 | 3.67 | 8\|8\|12 | 0 | 0 | 0 | 0 | 0 | −33.63 | 0.39 | 0.92 |
| O75131 | CPNE3 | 5\|4\|6 | 15 | 5 | 9\|5\|4 | 0 | 0 | 0 | 0 | 0 | −18.34 | 0.83 | 0.92 |
| P61006 | RAB8A | 5\|7\|6 | 18 | 6 | 8\|7\|7 | 0 | 0 | 0 | 0 | 0 | −21.63 | 0.82 | 0.92 |
| P08559-3 | PDHA1 | 14\|8\|8 | 30 | 10 | 11\|11\|11 | 0 | 0 | 0 | 0 | 0 | −30.24 | 0.91 | 0.91 |
| P21399 | ACO1 | 5\|3\|4 | 12 | 4 | 3\|6\|4 | 0 | 0 | 0 | 0 | 0 | −13.81 | 0.92 | 0.89 |
| P22626 | HNRNPA2B1 | 0\|5\|3 | 8 | 2.67 | 2\|5\|5 | 0 | 0 | 0 | 0 | 0 | −19.16 | 0.67 | 0.88 |
| Q15738 | NSDHL | 2\|5\|0 | 7 | 2.33 | 2\|3\|5 | 0 | 0 | 0 | 0 | 0 | −16.29 | 0.7 | 0.86 |
| Q15029-2 | EFTUD2 | 10\|9\|12 | 31 | 10.33 | 22\|19\|22 | 0 | 0 | 0 | 0 | 0 | −67.54 | 0.49 | 0.92 |
| Q7L2E3-3 | DHX30 | 7\|7\|9 | 23 | 7.67 | 13\|8\|10 | 0 | 0 | 0 | 0 | 0 | −29.47 | 0.74 | 0.92 |
| P54136 | RARS | 11\|11\|10 | 32 | 10.67 | 13\|13\|13 | 0 | 0 | 0 | 0 | 0 | −34.35 | 0.82 | 0.92 |
| Q02952-3 | AKAP12 | 2\|0\|5 | 7 | 2.33 | 4\|4\|6 | 0 | 0 | 0 | 0 | 0 | −22.13 | 0.5 | 0.89 |
| Q96PV6 | LENG8 | 2\|0\|2 | 4 | 1.33 | 2\|2\|2 | 0 | 0 | 0 | 0 | 0 | −10.49 | 0.67 | 0.92 |
| Q06830 | PRDX1 | 10\|4\|10 | 24 | 8 | 6\|5\|6 | 0 | 0 | 0 | 0 | 0 | −17.11 | 1.41 | 0.87 |
| O00116 | AGPS | 10\|6\|8 | 24 | 8 | 6\|5\|7 | 0 | 0 | 0 | 0 | 0 | −15.26 | 1.33 | 0.88 |
| Q03519-2 | TAP2 | 0\|3\|3 | 6 | 2 | 4\|2\|0 | 0 | 0.01 | 0 | 0.01 | 0 | −10.24 | 1 | 0.81 |
| P30520 | ADSS | 6\|8\|5 | 19 | 6.33 | 8\|6\|8 | 0 | 0 | 0 | 0 | 0 | −21.63 | 0.86 | 0.9 |
| Q15428 | SF3A2 | 6\|7\|6 | 19 | 6.33 | 3\|5\|10 | 0 | 0 | 0 | 0 | 0 | −14.98 | 1.06 | 0.9 |
| P10155-3 | TROVE2 | 2\|2\|2 | 6 | 2 | 2\|3\|2 | 0 | 0 | 0 | 0 | 0 | −8.15 | 0.86 | 0.92 |
| P49588 | AARS | 7\|6\|6 | 19 | 6.33 | 11\|10\|11 | 0 | 0 | 0 | 0 | 0 | −32.59 | 0.59 | 0.92 |
| Q9HCC0 | MCCC2 | 26\|19\|24 | 69 | 23 | 29\|26\|25 | 0 | 0 | 0 | 0 | 0 | −70.23 | 0.86 | 0.92 |
| P30419 | NMT1 | 3\|3\|4 | 10 | 3.33 | 5\|2\|4 | 0 | 0 | 0 | 0 | 0 | −11.41 | 0.91 | 0.88 |
| Q14498-2 | RBM39 | 7\|7\|6 | 20 | 6.67 | 7\|4\|9 | 0 | 0 | 0 | 0 | 0 | −17.62 | 1 | 0.9 |
| P60842 | EIF4A1 | 63\|51\|62 | 176 | 58.67 | 64\|70\|69 | 0 | 0 | 0 | 0 | 0 | −169.3 | 0.87 | 0.92 |
| Q96151-2 | RCC1L | 0\|2\|0 | 2 | 0.67 | 0\|2\|2 | 0 | 0.01 | 0 | 0.01 | 0 | −7.85 | 0.5 | 0.82 |
| P47755 | CAPZA2 | 5\|7\|5 | 17 | 5.67 | 4\|5\|6 | 0 | 0 | 0 | 0 | 0 | −13.18 | 1.13 | 0.89 |
| Q8TDD1 | DDX54 | 2\|2\|3 | 7 | 2.33 | 2\|5\|3 | 0 | 0 | 0 | 0 | 0 | −11.76 | 0.7 | 0.92 |
| P48507 | GCLM | 4\|4\|5 | 13 | 4.33 | 4\|2\|3 | 0 | 0 | 0 | 0 | 0 | −7.59 | 1.44 | 0.84 |
| P53985 | SLC16A1 | 6\|5\|4 | 15 | 5 | 5\|4\|5 | 0 | 0 | 0 | 0 | 0 | −13.48 | 1.07 | 0.89 |
| Q9BRX2 | PELO | 8\|5\|3 | 16 | 5.33 | 6\|5\|7 | 0 | 0 | 0 | 0 | 0 | −20.15 | 0.89 | 0.89 |
| Q96D46 | NMD3 | 5\|4\|5 | 14 | 4.67 | 6\|6\|6 | 0 | 0 | 0 | 0 | 0 | −18.31 | 0.78 | 0.92 |
| P08195-2 | SLC3A2 | 18\|18\|17 | 53 | 17.67 | 19\|23\|21 | 0 | 0 | 0 | 0 | 0 | −52.44 | 0.84 | 0.92 |
| P51659 | HSD17B4 | 8\|7\|7 | 22 | 7.33 | 7\|7\|8 | 0 | 0 | 0 | 0 | 0 | −18.51 | 1 | 0.9 |
| Q13586 | STIM1 | 0\|2\|0 | 2 | 0.67 | 3\|3\|0 | 0 | 0 | 0 | 0 | 0 | −10.39 | 0.33 | 0.92 |
| P56192 | MARS | 6\|4\|8 | 18 | 6 | 7\|8\|15 | 0 | 0 | 0 | 0 | 0 | −33.82 | 0.6 | 0.92 |
| P35573-2 | AGL | 0\|0\|2 | 2 | 0.67 | 2\|2\|0 | 0 | 0.01 | 0 | 0.01 | 0 | −7.85 | 0.5 | 0.82 |
| P20700 | LMNB1 | 20\|18\|16 | 54 | 18 | 17\|21\|19 | 0 | 0 | 0 | 0 | 0 | −46.71 | 0.95 | 0.92 |
| P55884 | EIF3B | 0\|2\|4 | 6 | 2 | 2\|3\|2 | 0 | 0.01 | 0 | 0.01 | 0 | −11.98 | 0.86 | 0.83 |
| P43246 | MSH2 | 8\|9\|9 | 26 | 8.67 | 10\|11\|17 | 0 | 0 | 0 | 0 | 0 | −36.62 | 0.68 | 0.92 |
| P07737 | PFN1 | 51\|49\|54 | 154 | 51.33 | 48\|47\|45 | 0 | 0 | 0 | 0 | 0 | −96.23 | 1.1 | 0.92 |
| P52294 | KPNA1 | 0\|0\|4 | 4 | 1.33 | 3\|2\|4 | 0 | 0 | 0 | 0 | 0 | −14.8 | 0.44 | 0.86 |
| P52292 | KPNA2 | 5\|4\|8 | 17 | 5.67 | 4\|0\|7 | 0 | 0.01 | 0 | 0.01 | 0 | −9.41 | 1.55 | 0.81 |
| P00966 | ASS1 | 19\|20\|27 | 66 | 22 | 30\|41\|39 | 0 | 0 | 0 | 0 | 0 | −109.1 | 0.6 | 0.92 |
| P62910 | RPL32 | 6\|6\|7 | 19 | 6.33 | 7\|5\|6 | 0 | 0 | 0 | 0 | 0 | −15.26 | 1.06 | 0.9 |
| P14618-2 | PKM | 73\|61\|64 | 198 | 66 | 72\|62\|72 | 0 | 0 | 0 | 0 | 0 | −156.69 | 0.96 | 0.92 |
| Q03518 | TAP1 | 4\|2\|0 | 6 | 2 | 3\|4\|3 | 0 | 0 | 0 | 0 | 0 | −16.29 | 0.6 | 0.88 |
| P62917 | RPL8 | 11\|11\|6 | 28 | 9.33 | 10\|6\|9 | 0 | 0 | 0 | 0 | 0 | −23.68 | 1.12 | 0.9 |
| O00425 | IGF2BP3 | 6\|6\|8 | 20 | 6.67 | 11\|10\|10 | 0 | 0 | 0 | 0 | 0 | −31.29 | 0.65 | 0.92 |
| Q9H832 | UBE2Z | 4\|0\|3 | 7 | 2.33 | 4\|2\|2 | 0 | 0 | 0 | 0 | 0 | −13.44 | 0.88 | 0.85 |
| Q15382 | RHEB | 5\|4\|3 | 12 | 4 | 6\|4\|3 | 0 | 0 | 0 | 0 | 0 | −13.81 | 0.92 | 0.89 |
| Q10471 | GALNT2 | 4\|2\|3 | 9 | 3 | 5\|3\|4 | 0 | 0 | 0 | 0 | 0 | −14.26 | 0.75 | 0.92 |
| Q00796 | SORD | 0\|3\|4 | 7 | 2.33 | 3\|4\|3 | 0 | 0 | 0 | 0 | 0 | −16.29 | 0.7 | 0.88 |
| Q9H0U4 | RAB1B | 13\|15\|15 | 43 | 14.33 | 16\|19\|18 | 0 | 0 | 0 | 0 | 0 | −46.72 | 0.81 | 0.92 |
| O00743-2 | PPP6C | 4\|3\|2 | 9 | 3 | 2\|3\|3 | 0 | 0 | 0 | 0 | 0 | −9.34 | 1.12 | 0.85 |
| Q9NTJ3 | SMC4 | 10\|11\|11 | 32 | 10.67 | 10\|12\|7 | 0 | 0 | 0 | 0 | 0 | −22.38 | 1.1 | 0.91 |
| O43683-2 | BUB1 | 2\|2\|0 | 4 | 1.33 | 3\|3\|4 | 0 | 0 | 0 | 0 | 0 | −16.29 | 0.4 | 0.92 |
| P35658-2 | NUP214 | 17\|15\|24 | 56 | 18.67 | 22\|26\|25 | 0 | 0 | 0 | 0 | 0 | −68.55 | 0.77 | 0.92 |
| P35268 | RPL22 | 9\|6\|6 | 21 | 7 | 7\|7\|7 | 0 | 0 | 0 | 0 | 0 | −18.77 | 1 | 0.9 |
| Q71U36-2 | TUBA1A | 81\|62\|75 | 218 | 72.67 | 77\|72\|76 | 0 | 0 | 0 | 0 | 0 | −178.23 | 0.97 | 0.92 |
| Q10713 | PMPCA | 3\|4\|3 | 10 | 3.33 | 3\|5\|4 | 0 | 0 | 0 | 0 | 0 | −12.55 | 0.83 | 0.92 |
| P00367 | GLUD1 | 2\|3\|3 | 8 | 2.67 | 2\|3\|3 | 0 | 0 | 0 | 0 | 0 | −9.34 | 1 | 0.86 |
| Q9UKN8 | GTF3C4 | 5\|0\|3 | 8 | 2.67 | 4\|5\|7 | 0 | 0 | 0 | 0 | 0 | −25.05 | 0.5 | 0.92 |
| Q3KQU3-4 | MAP7D1 | 11\|7\|6 | 24 | 8 | 8\|7\|9 | 0 | 0 | 0 | 0 | 0 | −22.42 | 1 | 0.9 |
| Q99623 | PHB2 | 4\|3\|7 | 14 | 4.67 | 8\|6\|9 | 0 | 0 | 0 | 0 | 0 | −26.82 | 0.61 | 0.92 |
| Q8N884 | MB21D1 | 0\|2\|0 | 2 | 0.67 | 0\|2\|2 | 0 | 0.01 | 0 | 0.01 | 0 | −7.85 | 0.5 | 0.82 |
| P11940-2 | PABPC1 | 4\|3\|2 | 9 | 3 | 4\|6\|5 | 0 | 0 | 0 | 0 | 0 | −18.21 | 0.6 | 0.92 |
| P28288 | ABCD3 | 4\|2\|3 | 9 | 3 | 4\|6\|6 | 0 | 0 | 0 | 0 | 0 | −19.58 | 0.56 | 0.92 |
| P23246 | SFPQ | 25\|29\|23 | 77 | 25.67 | 26\|23\|27 | 0 | 0 | 0 | 0 | 0 | −58.8 | 1.01 | 0.92 |
| Q7KZF4 | SND1 | 30\|26\|34 | 90 | 30 | 33\|32\|37 | 0 | 0 | 0 | 0 | 0 | −85.76 | 0.88 | 0.92 |
| P24752 | ACAT1 | 8\|13\|10 | 31 | 10.33 | 14\|12\|10 | 0 | 0 | 0 | 0 | 0 | −34.01 | 0.86 | 0.91 |
| Q99714 | HSD17B10 | 2\|2\|0 | 4 | 1.33 | 2\|2\|2 | 0 | 0 | 0 | 0 | 0 | −10.49 | 0.67 | 0.92 |
| Q9H307 | PNN | 2\|0\|3 | 5 | 1.67 | 3\|6\|3 | 0 | 0 | 0 | 0 | 0 | −19.16 | 0.42 | 0.92 |
| Q14157-1 | UBAP2L | 9\|11\|12 | 32 | 10.67 | 17\|6\|14 | 0 | 0 | 0 | 0 | 0 | −33.18 | 0.86 | 0.92 |
| P52701 | MSH6 | 6\|4\|4 | 14 | 4.67 | 7\|4\|4 | 0 | 0 | 0 | 0 | 0 | −14.62 | 0.93 | 0.89 |
| P11310 | ACADM | 3\|0\|3 | 6 | 2 | 4\|3\|0 | 0 | 0 | 0 | 0 | 0 | −11.81 | 0.86 | 0.84 |
| Q5T9A4 | ATAD3B | 5\|6\|5 | 16 | 5.33 | 5\|5\|5 | 0 | 0 | 0 | 0 | 0 | −13.18 | 1.07 | 0.89 |
| P43686-2 | PSMC4 | 4\|5\|7 | 16 | 5.33 | 5\|7\|6 | 0 | 0 | 0 | 0 | 0 | −18.31 | 0.89 | 0.9 |
| Q14166 | TTLL12 | 4\|4\|7 | 15 | 5 | 8\|5\|9 | 0 | 0 | 0 | 0 | 0 | −23.44 | 0.68 | 0.92 |
| P10809 | HSPD1 | 12\|22\|22 | 56 | 18.67 | 24\|19\|27 | 0 | 0 | 0 | 0 | 0 | −70.52 | 0.8 | 0.92 |
| Q7Z6E9-2 | RBBP6 | 4\|4\|7 | 15 | 5 | 10\|10\|12 | 0 | 0 | 0 | 0 | 0 | −36.77 | 0.47 | 0.92 |
| Q16836 | HADH | 5\|4\|5 | 14 | 4.67 | 6\|6\|3 | 0 | 0 | 0 | 0 | 0 | −14.62 | 0.93 | 0.92 |
| Q15043-2 | SLC39A14 | 0\|0\|2 | 2 | 0.67 | 2\|2\|0 | 0 | 0.01 | 0 | 0.01 | 0 | −7.85 | 0.5 | 0.82 |
| Q9UJU6-2 | DBNL | 5\|4\|5 | 14 | 4.67 | 4\|4\|5 | 0 | 0 | 0 | 0 | 0 | −12.29 | 1.08 | 0.89 |
| Q16543 | CDC37 | 4\|3\|6 | 13 | 4.33 | 5\|6\|4 | 0 | 0 | 0 | 0 | 0 | −16.28 | 0.87 | 0.89 |
| Q13257 | MAD2L1 | 3\|4\|4 | 11 | 3.67 | 3\|2\|4 | 0 | 0 | 0 | 0 | 0 | −9.04 | 1.22 | 0.86 |
| P49368 | CCT3 | 29\|28\|23 | 80 | 26.67 | 36\|36\|41 | 0 | 0 | 0 | 0 | 0 | −105.12 | 0.71 | 0.92 |
| P32119 | PRDX2 | 6\|7\|4 | 17 | 5.67 | 6\|5\|3 | 0 | 0 | 0 | 0 | 0 | −13.48 | 1.21 | 0.88 |
| Q07866-8 | KLC1 | 5\|0\|4 | 9 | 3 | 3\|4\|2 | 0 | 0 | 0 | 0 | 0 | −14.8 | 1 | 0.85 |
| P19013 | KRT4 | 5\|3\|0 | 8 | 2.67 | 4\|2\|3 | 0 | 0 | 0 | 0 | 0 | −14.8 | 0.89 | 0.85 |
| Q8IZP0-2 | ABI1 | 9\|11\|12 | 32 | 10.67 | 10\|10\|8 | 0 | 0 | 0 | 0 | 0 | −22.66 | 1.14 | 0.9 |
| P26196 | DDX6 | 5\|6\|7 | 18 | 6 | 4\|5\|4 | 0 | 0 | 0 | 0 | 0 | −10.83 | 1.38 | 0.87 |
| Q9BUQ8 | DDX23 | 2\|3\|3 | 8 | 2.67 | 8\|2\|7 | 0 | 0 | 0 | 0 | 0 | −20.77 | 0.47 | 0.92 |
| Q9BWE0 | REPIN1 | 0\|5\|3 | 8 | 2.67 | 5\|3\|4 | 0 | 0 | 0 | 0 | 0 | −19.16 | 0.67 | 0.88 |
| Q9BWD1 | ACAT2 | 10\|7\|7 | 24 | 8 | 13\|10\|11 | 0 | 0 | 0 | 0 | 0 | −33.29 | 0.71 | 0.92 |
| Q9Y5M8 | SRPRB | 3\|4\|3 | 10 | 3.33 | 6\|5\|4 | 0 | 0 | 0 | 0 | 0 | −16.28 | 0.67 | 0.92 |
| P33992 | MCM5 | 15\|20\|15 | 50 | 16.67 | 18\|15\|20 | 0 | 0 | 0 | 0 | 0 | −43.45 | 0.94 | 0.91 |
| P33993 | MCM7 | 30\|27\|29 | 86 | 28.67 | 29\|32\|31 | 0 | 0 | 0 | 0 | 0 | −71.88 | 0.93 | 0.92 |
| P33991 | MCM4 | 11\|18\|18 | 47 | 15.67 | 19\|21\|12 | 0 | 0 | 0 | 0 | 0 | −48.97 | 0.9 | 0.91 |
| P50914 | RPL14 | 3\|3\|3 | 9 | 3 | 3\|5\|5 | 0 | 0 | 0 | 0 | 0 | −13.81 | 0.69 | 0.92 |
| P49755 | TMED10 | 3\|3\|4 | 10 | 3.33 | 3\|4\|6 | 0 | 0 | 0 | 0 | 0 | −13.81 | 0.77 | 0.92 |
| P49756 | RBM25 | 0\|4\|3 | 7 | 2.33 | 4\|5\|4 | 0 | 0 | 0 | 0 | 0 | −20.66 | 0.54 | 0.92 |
| O75534-2 | CSDE1 | 10\|8\|15 | 33 | 11 | 12\|11\|9 | 0 | 0 | 0 | 0 | 0 | −29.02 | 1.03 | 0.91 |
| P49591 | SARS | 7\|6\|5 | 18 | 6 | 5\|4\|6 | 0 | 0 | 0 | 0 | 0 | −13.18 | 1.2 | 0.89 |
| Q86V48-2 | LUZP1 | 0\|4\|4 | 8 | 2.67 | 4\|4\|7 | 0 | 0 | 0 | 0 | 0 | −23.55 | 0.53 | 0.92 |
| P61586 | RHOA | 0\|6\|4 | 10 | 3.33 | 4\|4\|5 | 0 | 0 | 0 | 0 | 0 | −20.66 | 0.77 | 0.88 |
| Q14938-6 | NFIX | 0\|0\|2 | 2 | 0.67 | 2\|0\|2 | 0 | 0.01 | 0 | 0.01 | 0 | −7.85 | 0.5 | 0.82 |
| Q9H0D6 | XRN2 | 5\|7\|7 | 19 | 6.33 | 5\|7\|5 | 0 | 0 | 0 | 0 | 0 | −15.55 | 1.12 | 0.89 |
| O43172-2 | PRPF4 | 8\|4\|6 | 18 | 6 | 9\|8\|10 | 0 | 0 | 0 | 0 | 0 | −30.01 | 0.67 | 0.92 |
| Q7Z417 | NUFIP2 | 3\|3\|3 | 9 | 3 | 4\|8\|5 | 0 | 0 | 0 | 0 | 0 | −18.88 | 0.53 | 0.92 |
| P16401 | HIST1H1B | 3\|4\|5 | 12 | 4 | 8\|6\|5 | 0 | 0 | 0 | 0 | 0 | −21.48 | 0.63 | 0.92 |
| Q9UBX3 | SLC25A10 | 5\|5\|5 | 15 | 5 | 2\|5\|4 | 0 | 0 | 0 | 0 | 0 | −8.5 | 1.36 | 0.86 |
| Q9HC07 | TMEM165 | 4\|2\|3 | 9 | 3 | 2\|3\|3 | 0 | 0 | 0 | 0 | 0 | −9.34 | 1.12 | 0.85 |
| Q9NR12 | PDLIM7 | 15\|13\|12 | 40 | 13.33 | 13\|12\|10 | 0 | 0 | 0 | 0 | 0 | −26.53 | 1.14 | 0.91 |
| P28838-2 | LAP3 | 8\|7\|12 | 27 | 9 | 5\|6\|6 | 0 | 0 | 0 | 0 | 0 | −12.64 | 1.59 | 0.84 |
| P35613-2 | BSG | 8\|5\|8 | 21 | 7 | 5\|7\|5 | 0 | 0 | 0 | 0 | 0 | −15.55 | 1.24 | 0.89 |
| Q86X55-1 | CARM1 | 5\|4\|4 | 13 | 4.33 | 5\|5\|2 | 0 | 0 | 0 | 0 | 0 | −11.11 | 1.08 | 0.88 |
| O95782-2 | AP2A1 | 5\|7\|5 | 17 | 5.67 | 6\|5\|7 | 0 | 0 | 0 | 0 | 0 | −16.7 | 0.94 | 0.9 |
| O94979-6 | SEC31A | 6\|7\|7 | 20 | 6.67 | 4\|12\|9 | 0 | 0 | 0 | 0 | 0 | −23.45 | 0.8 | 0.92 |
| P13010 | XRCC5 | 38\|36\|43 | 117 | 39 | 45\|51\|49 | 0 | 0 | 0 | 0 | 0 | −122.42 | 0.81 | 0.92 |
| Q96IU4 | ABHD14B | 3\|3\|3 | 9 | 3 | 2\|3\|2 | 0 | 0 | 0 | 0 | 0 | −6.68 | 1.29 | 0.84 |
| Q9BW92 | TARS2 | 2\|0\|2 | 4 | 1.33 | 3\|2\|3 | 0 | 0 | 0 | 0 | 0 | −13.44 | 0.5 | 0.92 |
| P27824 | CANX | 10\|8\|8 | 26 | 8.67 | 6\|6\|8 | 0 | 0 | 0 | 0 | 0 | −14.71 | 1.3 | 0.89 |
| P40763-3 | STAT3 | 9\|7\|8 | 24 | 8 | 14\|10\|14 | 0 | 0 | 0 | 0 | 0 | −38.47 | 0.63 | 0.92 |
| P62906 | RPL10A | 10\|7\|10 | 27 | 9 | 10\|11\|11 | 0 | 0 | 0 | 0 | 0 | −30.74 | 0.84 | 0.92 |
| P83731 | RPL24 | 8\|9\|7 | 24 | 8 | 6\|5\|7 | 0 | 0 | 0 | 0 | 0 | −13.81 | 1.33 | 0.89 |
| O95071-2 | UBR5 | 3\|2\|3 | 8 | 2.67 | 12\|8\|7 | 0 | 0 | 0 | 0 | 0 | −34.75 | 0.3 | 0.92 |
| P28070 | PSMB4 | 4\|3\|3 | 10 | 3.33 | 4\|3\|4 | 0 | 0 | 0 | 0 | 0 | −11.41 | 0.91 | 0.88 |
| P52565 | ARHGDIA | 11\|5\|12 | 28 | 9.33 | 8\|11\|10 | 0 | 0 | 0 | 0 | 0 | −30.63 | 0.97 | 0.91 |
| Q15393 | SF3B3 | 4\|2\|4 | 10 | 3.33 | 7\|4\|2 | 0 | 0 | 0 | 0 | 0 | −15.52 | 0.77 | 0.92 |
| Q96T51 | RUFY1 | 2\|2\|3 | 7 | 2.33 | 4\|2\|0 | 0 | 0.01 | 0 | 0.01 | 0 | −6.72 | 1.17 | 0.83 |
| Q9Y2W1 | THRAP3 | 6\|4\|7 | 17 | 5.67 | 6\|9\|8 | 0 | 0 | 0 | 0 | 0 | −24.75 | 0.74 | 0.92 |
| O60563 | CCNT1 | 4\|3\|8 | 15 | 5 | 5\|4\|6 | 0 | 0 | 0 | 0 | 0 | −16.28 | 1 | 0.88 |
| Q9UMR2 | DDX19B | 4\|3\|5 | 12 | 4 | 7\|2\|7 | 0 | 0 | 0 | 0 | 0 | −17.47 | 0.75 | 0.92 |
| P35908 | KRT2 | 38\|32\|20 | 90 | 30 | 41\|51\|33 | 0 | 0 | 0 | 0 | 0 | −126.75 | 0.72 | 0.92 |
| Q8IY37 | DHX37 | 0\|0\|2 | 2 | 0.67 | 2\|2\|2 | 0 | 0 | 0 | 0 | 0 | −10.49 | 0.33 | 0.92 |
| P53004 | BLVRA | 0\|3\|4 | 7 | 2.33 | 4\|2\|3 | 0 | 0 | 0 | 0 | 0 | −14.8 | 0.78 | 0.86 |
| Q8N6H7 | ARFGAP2 | 4\|4\|3 | 11 | 3.67 | 5\|4\|3 | 0 | 0 | 0 | 0 | 0 | −12.55 | 0.92 | 0.92 |
| Q9Y305-3 | ACOT9 | 3\|7\|9 | 19 | 6.33 | 5\|7\|3 | 0 | 0 | 0 | 0 | 0 | −16.28 | 1.27 | 0.86 |
| P00558 | PGK1 | 31\|30\|31 | 92 | 30.67 | 31\|29\|28 | 0 | 0 | 0 | 0 | 0 | −62.68 | 1.05 | 0.92 |
| Q9Y2Z0-2 | SUGT1 | 2\|3\|2 | 7 | 2.33 | 2\|3\|4 | 0 | 0 | 0 | 0 | 0 | −10.47 | 0.78 | 0.92 |
| Q14137-2 | BOP1 | 5\|2\|0 | 7 | 2.33 | 0\|4\|4 | 0 | 0.01 | 0 | 0.01 | 0 | −13.27 | 0.88 | 0.83 |
| Q13428-3 | TCOF1 | 0\|0\|4 | 4 | 1.33 | 14\|15\|8 | 0 | 0 | 0 | 0 | 0 | −56.15 | 0.11 | 0.92 |
| Q9BY77-2 | POLDIP3 | 3\|3\|4 | 10 | 3.33 | 3\|2\|3 | 0 | 0 | 0 | 0 | 0 | −7.86 | 1.25 | 0.85 |
| Q13428-7 | TCOF1 | 0\|0\|4 | 4 | 1.33 | 14\|16\|7 | 0 | 0 | 0 | 0 | 0 | −55.94 | 0.11 | 0.92 |
| Q05048 | CSTF1 | 5\|3\|3 | 11 | 3.67 | 4\|5\|4 | 0 | 0 | 0 | 0 | 0 | −13.81 | 0.85 | 0.89 |
| Q5VT06 | CEP350 | 14\|17\|24 | 55 | 18.33 | 35\|43\|36 | 0 | 0 | 0 | 0 | 0 | −125.55 | 0.48 | 0.92 |
| Q92621 | NUP205 | 2\|0\|3 | 5 | 1.67 | 2\|3\|3 | 0 | 0 | 0 | 0 | 0 | −13.44 | 0.63 | 0.87 |
| P09382 | LGALS1 | 3\|0\|4 | 7 | 2.33 | 3\|3\|4 | 0 | 0 | 0 | 0 | 0 | −16.29 | 0.7 | 0.88 |
| P53396 | ACLY | 46\|44\|43 | 133 | 44.33 | 44\|42\|45 | 0 | 0 | 0 | 0 | 0 | −94.34 | 1.02 | 0.92 |
| O76021 | RSL1D1 | 2\|2\|2 | 6 | 2 | 7\|5\|5 | 0 | 0 | 0 | 0 | 0 | −20.94 | 0.35 | 0.92 |
| Q9NYB9-2 | ABI2 | 3\|2\|3 | 8 | 2.67 | 4\|6\|3 | 0 | 0 | 0 | 0 | 0 | −15.6 | 0.62 | 0.92 |
| P41227 | NAA10 | 3\|6\|5 | 14 | 4.67 | 5\|3\|6 | 0 | 0 | 0 | 0 | 0 | −15.06 | 1 | 0.89 |
| P29353-3 | SHC1 | 7\|4\|6 | 17 | 5.67 | 6\|6\|8 | 0 | 0 | 0 | 0 | 0 | −20.88 | 0.85 | 0.9 |
| P49207 | RPL34 | 4\|5\|3 | 12 | 4 | 5\|4\|3 | 0 | 0 | 0 | 0 | 0 | −12.55 | 1 | 0.88 |
| O75717 | WDHD1 | 0\|0\|2 | 2 | 0.67 | 0\|3\|5 | 0 | 0 | 0 | 0 | 0 | −13.21 | 0.25 | 0.92 |
| P47897-2 | QARS | 3\|4\|0 | 7 | 2.33 | 4\|5\|0 | 0 | 0 | 0 | 0 | 0 | −14.48 | 0.78 | 0.86 |
| Q8IWR0 | ZC3H7A | 3\|3\|5 | 11 | 3.67 | 3\|4\|3 | 0 | 0 | 0 | 0 | 0 | −10.22 | 1.1 | 0.86 |
| Q02809 | PLOD1 | 14\|11\|18 | 43 | 14.33 | 11\|8\|7 | 0 | 0 | 0 | 0 | 0 | −17.44 | 1.65 | 0.87 |
| Q9HCS7 | XAB2 | 0\|0\|2 | 2 | 0.67 | 50\|55\|51 | 0 | 0 | 0 | 0 | 0 | −233.9 | 0.01 | 0.92 |
| Q9NR50-3 | EIF2B3 | 3\|0\|3 | 6 | 2 | 4\|3\|4 | 0 | 0 | 0 | 0 | 0 | −17.76 | 0.55 | 0.92 |
| P06899 | HIST1H2BJ | 3\|3\|4 | 10 | 3.33 | 6\|9\|6 | 0 | 0 | 0 | 0 | 0 | −24.11 | 0.48 | 0.92 |
| P07384 | CAPN1 | 0\|3\|3 | 6 | 2 | 4\|5\|0 | 0 | 0 | 0 | 0 | 0 | −14.48 | 0.67 | 0.92 |
| Q9NZ01 | TECR | 5\|2\|5 | 12 | 4 | 5\|5\|5 | 0 | 0 | 0 | 0 | 0 | −18.21 | 0.8 | 0.92 |
| Q16531 | DDB1 | 3\|3\|4 | 10 | 3.33 | 3\|5\|8 | 0 | 0 | 0 | 0 | 0 | −17.6 | 0.63 | 0.92 |
| P06576 | ATP5B | 7\|12\|7 | 26 | 8.67 | 9\|9\|11 | 0 | 0 | 0 | 0 | 0 | −26.97 | 0.9 | 0.91 |
| O75150 | RNF40 | 3\|5\|3 | 11 | 3.67 | 4\|4\|5 | 0 | 0 | 0 | 0 | 0 | −13.81 | 0.85 | 0.89 |
| O75153 | CLUH | 6\|3\|4 | 13 | 4.33 | 8\|4\|5 | 0 | 0 | 0 | 0 | 0 | −18.88 | 0.76 | 0.9 |
| O75152 | ZC3H11A | 0\|0\|2 | 2 | 0.67 | 2\|3\|3 | 0 | 0 | 0 | 0 | 0 | −13.44 | 0.25 | 0.92 |
| Q02218-2 | OGDH | 2\|0\|0 | 2 | 0.67 | 2\|4\|5 | 0 | 0 | 0 | 0 | 0 | −17.76 | 0.18 | 0.92 |
| Q15717 | ELAVL1 | 7\|9\|11 | 27 | 9 | 10\|8\|9 | 0 | 0 | 0 | 0 | 0 | −24.48 | 1 | 0.91 |
| P07954-2 | FH | 2\|2\|4 | 8 | 2.67 | 2\|4\|4 | 0 | 0 | 0 | 0 | 0 | −11.76 | 0.8 | 0.88 |
| O60749-2 | SNX2 | 2\|0\|0 | 2 | 0.67 | 2\|4\|3 | 0 | 0 | 0 | 0 | 0 | −14.8 | 0.22 | 0.92 |
| O43432-4 | EIF4G3 | 2\|0\|5 | 7 | 2.33 | 2\|7\|4 | 0 | 0 | 0 | 0 | 0 | −20.58 | 0.54 | 0.89 |
| P36404-2 | ARL2 | 2\|4\|3 | 9 | 3 | 2\|3\|3 | 0 | 0 | 0 | 0 | 0 | −9.34 | 1.12 | 0.85 |
| Q96FW1 | OTUB1 | 6\|4\|5 | 15 | 5 | 6\|8\|8 | 0 | 0 | 0 | 0 | 0 | −23.44 | 0.68 | 0.92 |
| P36578 | RPL4 | 44\|46\|42 | 132 | 44 | 39\|40\|44 | 0 | 0 | 0 | 0 | 0 | −86.4 | 1.07 | 0.92 |
| P63173 | RPL38 | 12\|12\|12 | 36 | 12 | 13\|10\|9 | 0 | 0 | 0 | 0 | 0 | −23.01 | 1.12 | 0.91 |
| P30508 | HLA-C | 5\|5\|0 | 10 | 3.33 | 6\|6\|4 | 0 | 0 | 0 | 0 | 0 | −25.05 | 0.63 | 0.92 |
| Q05639 | EEF1A2 | 24\|23\|20 | 67 | 22.33 | 27\|25\|25 | 0 | 0 | 0 | 0 | 0 | −64.78 | 0.87 | 0.92 |
| Q9BY44 | EIF2A | 8\|7\|16 | 31 | 10.33 | 9\|5\|9 | 0 | 0 | 0 | 0 | 0 | −19.69 | 1.35 | 0.86 |
| P12004 | PCNA | 11\|9\|8 | 28 | 9.33 | 9\|11\|16 | 0 | 0 | 0 | 0 | 0 | −34.08 | 0.78 | 0.92 |
| Q9BQ67 | GRWD1 | 3\|3\|4 | 10 | 3.33 | 5\|4\|6 | 0 | 0 | 0 | 0 | 0 | −16.28 | 0.67 | 0.92 |
| P07951-3 | TPM2 | 0\|0\|2 | 2 | 0.67 | 2\|2\|2 | 0 | 0 | 0 | 0 | 0 | −10.49 | 0.33 | 0.92 |
| O95758-1 | PTBP3 | 2\|0\|0 | 2 | 0.67 | 3\|2\|0 | 0 | 0 | 0 | 0 | 0 | −9.18 | 0.4 | 0.84 |
| P22314-2 | UBA1 | 3\|4\|5 | 12 | 4 | 4\|4\|3 | 0 | 0 | 0 | 0 | 0 | −11.41 | 1.09 | 0.87 |

[symbol]

indicates data missing or illegible when filed

TABLE 12
SAINT N-Turbo vs. Control
N-term Bait

| | | | | | | | | | | | log- | Fold- | |
|---|---|---|---|---|---|---|---|---|---|---|---|---|---|
| Prey | PreyGene | Spec | SpecSum | AvgSpec | ctrlCounts | AvgP | MaxP | TopoAvgP | TopoMaxP | SaintScore | OddsScore | Change | BFDR |
| Q63HN8 | RNF213 | 46\|450\|44 | 1338 | 446 | 34\|39\|43 | 1 | 1 | 3 | 1 | 1 | 396.34 | 11.53 | 0 |
| Q16850-2 | CYP51A1 | 3\|4\|4 | 11 | 3.67 | 0\|0\|0 | 0.98 | 0.99 | 0.98 | 0.99 | 0.98 | 3.17 | 36.67 | 0 |
| O96013 | PAK4 | 3\|3\|5 | 11 | 3.67 | 0\|0\|0 | 0.97 | 3 | 0.97 | 1 | 0.97 | 3.17 | 36.67 | 0.01 |
| O15269 | SPTLC1 | 2\|4\|4 | 10 | 3.33 | 0\|0\|0 | 0.93 | 0.99 | 0.93 | 0.99 | 0.93 | 1.33 | 33.33 | 0.02 |
| Q86SQ0-2 | PHLDB2 | 3\|5\|2 | 10 | 3.33 | 0\|0\|0 | 0.92 | 1 | 0.92 | 1 | 0.92 | 1.33 | 33.33 | 0.03 |
| Q969X6-2 | UTP4 | 5\|3\|2 | 10 | 3.33 | 0\|0\|0 | 0.92 | 3 | 0.92 | 1 | 0.92 | 1.33 | 33.33 | 0.03 |
| ASYKK6 | CNOT1 | 3\|4\|2 | 9 | 3 | 0\|0\|0 | 0.91 | 0.99 | 0.91 | 0.99 | 0.91 | 1.33 | 30 | 0.05 |
| Q02750-2 | MAP2K1 | 2\|3\|4 | 9 | 3 | 0\|0\|0 | 0.91 | 0.99 | 0.91 | 0.99 | 0.91 | 1.33 | 30 | 0.05 |
| Q15773 | MLF2 | 2\|3\|3 | 8 | 2.67 | 0\|0\|0 | 0.9 | 0.96 | 0.9 | 0.96 | 0.9 | 1.33 | 26.67 | 0.06 |
| Q99755-3 | PIPSK1A | 2\|3\|3 | 8 | 2.67 | 0\|0\|0 | 0.9 | 0.96 | 0.9 | 0.96 | 0.9 | 1.33 | 26.67 | 0.06 |
| Q9NQC7-2 | CYLD | 2\|3\|2 | 7 | 2.33 | 0\|0\|0 | 0.85 | 0.96 | 0.85 | 0.96 | 0.85 | 1.33 | 23.33 | 0.07 |
| O75477 | ERLIN1 | 2\|2\|2 | 6 | 2 | 0\|0\|0 | 0.79 | 0.79 | 0.79 | 0.79 | 0.79 | 1.33 | 20 | 0.07 |
| Q14119 | VEZF1 | 8\|0\|5 | 13 | 4.33 | 0\|0\|0 | 0.67 | 1 | 0.67 | 1 | 0.67 | −3.21 | 43.33 | 0.08 |
| Q08AE8-2 | SPIRE1 | 4\|6\|0 | 10 | 3.33 | 0\|0\|0 | 0.66 | 3 | 0.66 | 1 | 0.66 | −3.21 | 33.33 | 0.1 |
| Q13045-2 | FLII | 4\|0\|3 | 7 | 2.33 | 0\|0\|0 | 0.65 | 0.99 | 0.65 | 0.99 | 0.65 | −3.21 | 23.33 | 0.12 |
| Q6P1L5 | FAM117B | 0\|3\|3 | 6 | 2 | 0\|0\|0 | 0.64 | 0.96 | 0.64 | 0.96 | 0.64 | −3.21 | 20 | 0.14 |
| Q6XZF7 | DNMBP | 3\|0\|3 | 6 | 2 | 0\|0\|0 | 0.64 | 0.96 | 0.64 | 0.96 | 0.64 | −3.21 | 20 | 0.14 |
| Q96CB8 | INTS12 | 0\|2\|5 | 7 | 2.33 | 0\|0\|0 | 0.6 | 1 | 0.6 | 1 | 0.6 | −3.21 | 23.33 | 0.16 |
| Q725Q1-7 | CPEB2 | 4\|2\|0 | 6 | 2 | 0\|0\|0 | 0.59 | 0.99 | 0.59 | 0.99 | 0.59 | −3.21 | 20 | 0.18 |
| Q15154-4 | PCM1 | 4\|0\|2 | 6 | 2 | 0\|0\|0 | 0.59 | 0.99 | 0.59 | 0.99 | 0.59 | −3.21 | 20 | 0.18 |
| P22061 | PCMT1 | 0\|4\|2 | 6 | 2 | 0\|0\|0 | 0.59 | 0.99 | 0.59 | 0.99 | 0.59 | −3.21 | 20 | 0.18 |
| P30533 | LRPAP1 | 2\|0\|4 | 6 | 2 | 0\|0\|0 | 0.59 | 0.99 | 0.59 | 0.99 | 0.59 | −3.21 | 20 | 0.18 |
| Q13131 | PRKAA1 | 3\|0\|2 | 5 | 1.67 | 0\|0\|0 | 0.58 | 0.96 | 0.58 | 0.96 | 0.58 | −3.21 | 16.67 | 0.22 |
| Q99456 | KRT12 | 2\|3\|0 | 5 | 1.67 | 0\|0\|0 | 0.58 | 0.96 | 0.58 | 0.96 | 0.58 | −3.21 | 16.67 | 0.22 |
| Q92616 | GCN1 | 0\|3\|2 | 5 | 1.67 | 0\|0\|0 | 0.58 | 0.96 | 0.58 | 0.96 | 0.58 | −3.21 | 16.67 | 0.22 |
| Q9UHD2 | TBK1 | 2\|3\|0 | 5 | 1.67 | 0\|0\|0 | 0.58 | 0.96 | 0.58 | 0.96 | 0.58 | −3.21 | 16.67 | 0.22 |
| P62857 | RPS28 | 2\|0\|3 | 5 | 1.67 | 0\|0\|0 | 0.58 | 0.96 | 0.58 | 0.96 | 0.58 | −3.21 | 16.67 | 0.22 |
| Q9HD45 | TM9SF3 | 2\|3\|0 | 5 | 1.67 | 0\|0\|0 | 0.58 | 0.96 | 0.58 | 0.96 | 0.58 | −3.21 | 16.67 | 0.22 |
| O60716-21 | CTNND1 | 2\|0\|3 | 5 | 1.67 | 0\|0\|0 | 0.58 | 0.96 | 0.58 | 0.96 | 0.58 | −3.21 | 16.67 | 0.22 |
| P82650 | MRPS22 | 2\|0\|3 | 5 | 1.67 | 0\|0\|0 | 0.58 | 0.96 | 0.58 | 0.96 | 0.58 | −3.21 | 16.67 | 0.22 |
| P06789 | P06789 | 0\|3\|2 | 5 | 1.67 | 0\|0\|0 | 0.58 | 0.96 | 0.58 | 0.96 | 0.58 | −3.21 | 16.67 | 0.22 |
| P31751-2 | AKT2 | 2\|3\|0 | 5 | 1.67 | 0\|0\|0 | 0.58 | 0.96 | 0.58 | 0.96 | 0.58 | −3.21 | 16.67 | 0.22 |
| A6NED2 | RCCD1 | 2\|0\|3 | 5 | 1.67 | 0\|0\|0 | 0.58 | 0.96 | 0.58 | 0.96 | 0.58 | −3.21 | 16.67 | 0.22 |
| Q8NB46 | ANKRD52 | 0\|3\|2 | 5 | 1.67 | 0\|0\|0 | 0.58 | 0.96 | 0.58 | 0.96 | 0.58 | −3.21 | 16.67 | 0.22 |
| Q9UGR2 | ZC3H78 | 3\|2\|0 | 5 | 1.67 | 0\|0\|0 | 0.58 | 0.96 | 0.58 | 0.96 | 0.58 | −3.21 | 16.67 | 0.22 |
| Q92625 | ANKS1A | 0\|2\|3 | 5 | 1.67 | 0\|0\|0 | 0.58 | 0.96 | 0.58 | 0.96 | 0.58 | −3.21 | 16.67 | 0.22 |
| Q9NX74 | DUS2 | 2\|2\|0 | 4 | 1.33 | 0\|0\|0 | 0.53 | 0.79 | 0.53 | 0.79 | 0.53 | −3.21 | 13.33 | 0.29 |
| Q9Y232-3 | CDYL | 2\|0\|2 | 4 | 1.33 | 0\|0\|0 | 0.53 | 0.79 | 0.53 | 0.79 | 0.53 | −3.21 | 13.33 | 0.29 |
| O14925 | TIMM23 | 2\|2\|0 | 4 | 1.33 | 0\|0\|0 | 0.53 | 0.79 | 0.53 | 0.79 | 0.53 | −3.21 | 13.33 | 0.29 |
| Q9UI30 | TRMT112 | 2\|0\|2 | 4 | 1.33 | 0\|0\|0 | 0.53 | 0.79 | 0.53 | 0.79 | 0.53 | −3.21 | 13.33 | 0.29 |
| Q9UN86-2 | G3BP2 | 2\|0\|2 | 4 | 1.33 | 0\|0\|0 | 0.53 | 0.79 | 0.53 | 0.79 | 0.53 | −3.21 | 13.33 | 0.29 |
| P02662 | P02662 | 2\|0\|2 | 0 | 1.33 | 0\|0\|0 | 0.53 | 0.79 | 0.53 | 0.79 | 0.53 | −3.21 | 13.33 | 0.29 |
| Q9Y3P9 | RABGAP1 | 2\|2\|0 | 4 | 1.33 | 0\|0\|0 | 0.53 | 0.79 | 0.53 | 0.79 | 0.53 | −3.21 | 13.33 | 0.29 |
| P14373-2 | TRIM27 | 2\|2\|0 | 4 | 1.33 | 0\|0\|0 | 0.53 | 0.79 | 0.53 | 0.79 | 0.53 | −3.21 | 13.33 | 0.29 |
| P45973 | CBXS | 2\|2\|0 | 4 | 1.33 | 0\|0\|0 | 0.53 | 0.79 | 0.53 | 0.79 | 0.53 | −3.21 | 13.33 | 0.29 |
| Q9H0W8-2 | SMG9 | 0\|2\|2 | 4 | 1.33 | 0\|0\|0 | 0.53 | 0.79 | 0.53 | 0.79 | 0.53 | −3.21 | 13.33 | 0.29 |
| P78312-4 | FAM193A | 2\|0\|2 | 4 | 1.33 | 0\|0\|0 | 0.53 | 0.79 | 0.53 | 0.79 | 0.53 | −3.21 | 13.33 | 0.29 |
| P14635-2 | CCNB1 | 2\|0\|2 | 4 | 1.33 | 0\|0\|0 | 0.53 | 0.79 | 0.53 | 0.79 | 0.53 | −3.21 | 13.33 | 0.29 |
| P08574 | CYC1 | 2\|0\|2 | 4 | 1.33 | 0\|0\|0 | 0.53 | 0.79 | 0.53 | 0.79 | 0.53 | −3.21 | 13.33 | 0.29 |
| Q9Y6B6 | SAR1B | 2\|2\|0 | 0 | 1.33 | 0\|0\|0 | 0.53 | 0.79 | 0.53 | 0.79 | 0.53 | −3.21 | 13.33 | 0.29 |
| O75128 | COBL | 2\|2\|0 | 4 | 1.33 | 0\|0\|0 | 0.53 | 0.79 | 0.53 | 0.79 | 0.53 | −3.21 | 13.33 | 0.29 |
| Q99755-4 | PIPSK1A | 0\|2\|2 | 4 | 1.33 | 0\|0\|0 | 0.53 | 0.79 | 0.53 | 0.79 | 0.53 | −3.21 | 13.33 | 0.29 |
| O60493-4 | SNX3 | 2\|2\|0 | 4 | 1.33 | 0\|0\|0 | 0.53 | 0.79 | 0.53 | 0.79 | 0.53 | −3.21 | 13.33 | 0.29 |
| Q15155 | NOMO1 | 2\|2\|0 | 4 | 1.33 | 0\|0\|0 | 0.53 | 0.79 | 0.53 | 0.79 | 0.53 | −3.21 | 13.33 | 0.29 |
| Q9BSH4 | TACO1 | 2\|0\|2 | 4 | 1.33 | 0\|0\|0 | 0.53 | 0.79 | 0.53 | 0.79 | 0.53 | −3.21 | 13.33 | 0.29 |
| Q7Z739 | YTHDF3 | 2\|0\|2 | 4 | 1.33 | 0\|0\|0 | 0.53 | 0.79 | 0.53 | 0.79 | 0.53 | −3.21 | 13.33 | 0.29 |
| P08865 | RPSA | 6\|4\|4 | 14 | 4.67 | 0\|0\|2 | 0.52 | 0.75 | 0.52 | 0.75 | 0.52 | −0.37 | 7 | 0.36 |
| O75592-2 | MYCBP2 | 2\|5\|5 | 12 | 4 | 0\|2\|0 | 0.43 | 0.59 | 0.43 | 0.59 | 0.43 | −2.03 | 6 | 0.36 |
| Q6P2H3-2 | CEP85 | 8\|3\|2 | 13 | 4.33 | 2\|0\|0 | 0.43 | 0.92 | 0.43 | 0.92 | 0.43 | −2.03 | 6.5 | 0.36 |
| O95630-2 | STAMBP | 2\|8\|2 | 12 | 4 | 0\|0\|2 | 0.38 | 0.92 | 0.38 | 0.92 | 0.38 | −2.03 | 6 | 0.37 |
| P45974-2 | USPS | 6\|8\|9 | 23 | 7.67 | 4\|2\|0 | 0.37 | 0.58 | 0.37 | 0.58 | 0.37 | −1.89 | 3.83 | 0.37 |
| Q65193-2 | FAM111B | 4\|0\|0 | 4 | 1.33 | 0\|0\|0 | 0.33 | 0.99 | 0.33 | 0.99 | 0.33 | −3.21 | 13.33 | 0.38 |
| Q7Z4G4-3 | TRMT11 | 0\|4\|0 | 4 | 1.33 | 0\|0\|0 | 0.33 | 0.99 | 0.33 | 0.99 | 0.33 | −3.21 | 13.33 | 0.38 |
| P16989-2 | YBX3 | 0\|0\|4 | 4 | 1.33 | 0\|0\|0 | 0.33 | 0.99 | 0.33 | 0.99 | 0.33 | −3.21 | 13.33 | 0.38 |
| O8N108-2 | NCOA7 | 4\|0\|0 | 4 | 1.33 | 0\|0\|0 | 0.33 | 0.99 | 0.33 | 0.99 | 0.33 | −3.21 | 13.33 | 0.38 |
| Q00537 | CDK17 | 3\|0\|0 | 3 | 1 | 0\|0\|0 | 0.32 | 0.96 | 0.32 | 0.96 | 0.32 | −3.21 | 10 | 0.39 |
| Q04323 | UBXN | 3\|0\|0 | 3 | 1 | 0\|0\|0 | 0.32 | 0.96 | 0.32 | 0.96 | 0.32 | −3.21 | 10 | 0.39 |
| P98082-3 | DAB2 | 3\|0\|0 | 3 | 1 | 0\|0\|0 | 0.32 | 0.96 | 0.32 | 0.96 | 0.32 | −3.21 | 10 | 0.39 |
| P09001 | MRPL3 | 3\|0\|0 | 3 | 1 | 0\|0\|0 | 0.32 | 0.96 | 0.32 | 0.96 | 0.32 | −3.21 | 10 | 0.39 |
| Q9NZM1-2 | MYOF | 0\|3\|0 | 3 | 1 | 0\|0\|0 | 0.32 | 0.96 | 0.32 | 0.96 | 0.32 | −3.21 | 10 | 0.39 |
| Q13126-4 | MTAP | 0\|0\|3 | 3 | 1 | 0\|0\|0 | 0.32 | 0.96 | 0.32 | 0.96 | 0.32 | −3.21 | 10 | 0.39 |
| Q15208 | STK38 | 0\|3\|0 | 3 | 1 | 0\|0\|0 | 0.32 | 0.96 | 0.32 | 0.96 | 0.32 | −3.21 | 10 | 0.39 |
| Y6C9 | MTCH2 | 0\|0\|3 | 3 | 1 | 0\|0\|0 | 0.32 | 0.96 | 0.32 | 0.96 | 0.32 | −3.21 | 10 | 0.39 |
| Q9ULV3-5 | CIZ1 | 0\|0\|3 | 3 | 1 | 0\|0\|0 | 0.32 | 0.96 | 0.32 | 0.96 | 0.32 | −3.21 | 10 | 0.39 |
| Q12899 | TRIM26 | 0\|3\|0 | 3 | 1 | 0\|0\|0 | 0.32 | 0.96 | 0.32 | 0.96 | 0.32 | −3.21 | 10 | 0.39 |
| Q96SU4-5 | OSBPL9 | 3\|0\|0 | 3 | 1 | 0\|0\|0 | 0.32 | 0.96 | 0.32 | 0.96 | 0.32 | −3.21 | 10 | 0.39 |
| O95391 | SLU7 | 0\|0\|3 | 3 | 1 | 0\|0\|0 | 0.32 | 0.96 | 0.32 | 0.96 | 0.32 | −3.21 | 10 | 0.39 |
| Q9NX46 | ADPRHL2 | 3\|0\|0 | 3 | 1 | 0\|0\|0 | 0.32 | 0.96 | 0.32 | 0.96 | 0.32 | −3.21 | 10 | 0.39 |
| Q9NRG9-2 | AAAS | 0\|0\|3 | 3 | 1 | 0\|0\|0 | 0.32 | 0.96 | 0.32 | 0.96 | 0.32 | −3.21 | 10 | 0.39 |
| Q7L099-2 | RUFY3 | 0\|0\|3 | 3 | 3 | 010\|0 | 0.32 | 0.96 | 0.32 | 0.96 | 0.32 | −3.21 | 10 | 0.39 |
| Q6IN84 | MRM1 | 0\|3\|0 | 3 | 1 | 0\|0\|0 | 0.32 | 0.96 | 0.32 | 0.96 | 0.32 | −3.21 | 10 | 0.39 |
| P20618 | PSMB1 | 0\|0\|3 | 3 | 3 | 0\|0\|0 | 0.32 | 0.96 | 0.32 | 0.96 | 0.32 | −3.21 | 10 | 0.39 |
| Q13564-3 | NAE1 | 0\|0\|3 | 3 | 1 | 0\|0\|0 | 0.32 | 0.96 | 0.32 | 0.96 | 0.32 | −3.21 | 10 | 0.39 |
| Q13057 | COASY | 3\|0\|0 | 3 | 1 | 0\|0\|0 | 0.32 | 0.96 | 0.32 | 0.96 | 0.32 | −3.21 | 10 | 0.39 |
| O60547-2 | GMDS | 0\|0\|3 | 3 | 1 | 0\|0\|0 | 0.32 | 0.96 | 0.32 | 0.96 | 0.32 | −3.21 | 10 | 0.39 |
| Q9UM82 | SPATA2 | 4\|2\|4 | 10 | 3.33 | 2\|0\|0 | 0.31 | 0.41 | 0.31 | 0.41 | 0.31 | −2.03 | 5 | 0.46 |
| Q9UQ13-2 | SHOC2 | 3\|3\|4 | 10 | 3.33 | 2\|0\|0 | 0.3 | 0.41 | 0.3 | 0.41 | 0.3 | −1.16 | 5 | 0.46 |
| Q6ZNB6 | NFXL1 | 3\|2\|6 | 11 | 3.67 | 2\|0\|2 | 0.28 | 0.8 | 0.28 | 0.8 | 0.28 | −4.67 | 2.75 | 0.47 |
| O6P996-3 | PDXDC1 | 2\|0\|0 | 2 | 0.67 | 0\|0\|0 | 0.26 | 0.79 | 0.26 | 0.79 | 0.26 | −3.21 | 6.67 | 0.47 |
| Q96T60 | PNKP | 2\|0\|0 | 2 | 0.67 | 0\|0\|0 | 0.26 | 0.79 | 0.26 | 0.79 | 0.26 | −3.21 | 6.67 | 0.47 |
| Q9BUN8-2 | DERL1 | 0\|0\|2 | 2 | 0.67 | 0\|0\|0 | 0.26 | 0.79 | 0.26 | 0.79 | 0.26 | −3.21 | 6.67 | 0.47 |
| Q8NHH9-3 | ATL2 | 2\|0\|0 | 2 | 0.67 | 0\|0\|0 | 0.26 | 0.79 | 0.26 | 0.79 | 0.26 | −3.21 | 6.67 | 0.47 |
| O01433-2 | AMPD2 | 0\|2\|0 | 2 | 0.67 | 0\|0\|0 | 0.26 | 0.79 | 0.26 | 0.79 | 0.26 | −3.21 | 6.67 | 0.47 |
| P49593-2 | PPM1F | 2\|0\|0 | 2 | 0.67 | 0\|0\|0 | 0.26 | 0.79 | 0.26 | 0.79 | 0.26 | −3.21 | 6.67 | 0.47 |
| O14444-2 | CAPRIN1 | 2\|0\|0 | 2 | 0.67 | 0\|0\|0 | 0.26 | 0.79 | 0.26 | 0.79 | 0.26 | −3.21 | 6.67 | 0.47 |
| Q96FZ2 | HMCES | 0\|0\|2 | 2 | 0.67 | 0\|0\|0 | 0.26 | 0.79 | 0.26 | 0.79 | 0.26 | −3.21 | 6.67 | 0.47 |
| O15066 | KIF3B | 0\|0\|2 | 2 | 0.67 | 0\|0\|0 | 0.26 | 0.79 | 0.26 | 0.79 | 0.26 | −3.21 | 6.67 | 0.47 |
| Q9NVC6 | MED17 | 0\|2\|0 | 2 | 0.67 | 0\|0\|0 | 0.26 | 0.79 | 0.26 | 0.79 | 0.26 | −3.21 | 6.67 | 0.47 |
| Q9H4H8-2 | FAM83D | 0\|2\|0 | 2 | 0.67 | 0\|0\|0 | 0.26 | 0.79 | 0.26 | 0.79 | 0.26 | −3.21 | 6.67 | 0.47 |
| P19447 | ERCC3 | 2\|0\|0 | 2 | 0.67 | 0\|0\|0 | 0.26 | 0.79 | 0.26 | 0.79 | 0.26 | −3.21 | 6.67 | 0.47 |
| Q6PL18 | ATAD2 | 0\|2\|0 | 2 | 0.67 | 0\|0\|0 | 0.26 | 0.79 | 0.26 | 0.79 | 0.26 | −3.21 | 6.67 | 0.47 |
| P33897 | ABCD1 | 2\|0\|0 | 2 | 0.67 | 0\|0\|0 | 0.26 | 0.79 | 0.26 | 0.79 | 0.26 | −3.21 | 6.67 | 0.47 |
| Q8WWC4 | MAIP1 | 2\|0\|0 | 2 | 0.67 | 0\|0\|0 | 0.26 | 0.79 | 0.26 | 0.79 | 0.26 | −3.21 | 6.67 | 0.47 |
| Q96C19 | EFHD2 | 2\|0\|0 | 2 | 0.67 | 0\|0\|0 | 0.26 | 0.79 | 0.26 | 0.79 | 0.26 | −3.21 | 6.67 | 0.47 |
| Q96R06 | SPAG5 | 0\|0\|2 | 2 | 0.67 | 0\|0\|0 | 0.26 | 0.79 | 0.26 | 0.79 | 0.26 | −3.21 | 6.67 | 0.47 |
| Q9H788-2 | SH2D4A | 0\|2\|0 | 2 | 0.67 | 0\|0\|0 | 0.26 | 0.79 | 0.26 | 0.79 | 0.26 | −3.21 | 6.67 | 0.47 |
| Q9BTC8-2 | MTA3 | 0\|0\|2 | 2 | 0.67 | 0\|0\|0 | 0.26 | 0.79 | 0.26 | 0.79 | 0.26 | −3.21 | 6.67 | 0.47 |
| Q9HAD4 | WDR41 | 2\|0\|0 | 2 | 0.67 | 0\|0\|0 | 0.26 | 0.79 | 0.26 | 0.79 | 0.26 | −3.21 | 6.67 | 0.47 |
| Q9BQ13-2 | EIF2AK1 | 0\|2\|0 | 2 | 0.67 | 0\|0\|0 | 0.26 | 0.79 | 0.26 | 0.79 | 0.26 | −3.21 | 6.67 | 0.47 |
| 095985-3 | TOP3B | 2\|0\|0 | 2 | 0.67 | 0\|0\|0 | 0.26 | 0.79 | 0.26 | 0.79 | 0.26 | −3.21 | 6.67 | 0.47 |
| Q13442 | PDAP1 | 2\|0\|0 | 2 | 0.67 | 0\|0\|0 | 0.26 | 0.79 | 0.26 | 0.79 | 0.26 | −3.21 | 6.67 | 0.47 |
| Q9H3M7-2 | TXNIP | 2\|0\|0 | 2 | 0.67 | 0\|0\|0 | 0.26 | 0.79 | 0.26 | 0.79 | 0.26 | −3.21 | 6.67 | 0.47 |
| Q8N5A5-3 | ZGPAT | 0\|0\|2 | 2 | 0.67 | 0\|0\|0 | 0.26 | 0.79 | 0.26 | 0.79 | 0.26 | −3.21 | 6.67 | 0.47 |
| O00186 | STXBP3 | 0\|2\|0 | 2 | 0.67 | 0\|0\|0 | 0.26 | 0.79 | 0.26 | 0.79 | 0.26 | −3.21 | 6.67 | 0.47 |
| Q969ZO | TBRG4 | 2\|0\|0 | 2 | 0.67 | 0\|0\|0 | 0.26 | 0.79 | 0.26 | 0.79 | 0.26 | −3.21 | 6.67 | 0.47 |
| Q9BQA1-2 | WDR77 | 0\|0\|2 | 2 | 0.67 | 0\|0\|0 | 0.26 | 0.79 | 0.26 | 0.79 | 0.26 | −3.21 | 6.67 | 0.47 |
| P06858 | LPL | 0\|2\|0 | 2 | 0.67 | 0\|0\|0 | 0.26 | 0.79 | 0.26 | 0.79 | 0.26 | −3.21 | 6.67 | 0.47 |
| P50570-2 | DNM2 | 2\|0\|0 | 2 | 0.67 | 0\|0\|0 | 0.26 | 0.79 | 0.26 | 0.79 | 0.26 | −3.21 | 6.67 | 0.47 |
| Q9C0D2 | CEP295 | 0\|2\|0 | 2 | 0.67 | 0\|0\|0 | 0.26 | 0.79 | 0.26 | 0.79 | 0.26 | −3.21 | 6.67 | 0.47 |
| O95835-2 | LATS1 | 0\|0\|2 | 2 | 0.67 | 0\|0\|0 | 0.26 | 0.79 | 0.26 | 0.79 | 0.26 | −3.21 | 6.67 | 0.47 |
| Q9Y3S2 | ZNF330 | 0\|2\|0 | 2 | 0.67 | 0\|0\|0 | 0.26 | 0.79 | 0.26 | 0.79 | 0.26 | −3.21 | 6.67 | 0.47 |
| O95819-4 | MAP4K4 | 0\|0\|2 | 2 | 0.67 | 0\|0\|0 | 0.26 | 0.79 | 0.26 | 0.79 | 0.26 | −3.21 | 6.67 | 0.47 |
| Q9NUQ7 | UFSP2 | 2\|0\|0 | 2 | 0.67 | 0\|0\|0 | 0.26 | 0.79 | 0.26 | 0.79 | 0.26 | −3.21 | 6.67 | 0.47 |
| Q93074-3 | MED12 | 0\|2\|0 | 2 | 0.67 | 0\|0\|0 | 0.26 | 0.79 | 0.26 | 0.79 | 0.26 | −3.21 | 6.67 | 0.47 |
| Q9Y6V7 | DDX49 | 0\|0\|2 | 2 | 0.67 | 0\|0\|0 | 0.26 | 0.79 | 0.26 | 0.79 | 0.26 | −3.21 | 6.67 | 0.47 |
| Q8NCN5 | PDPR | 0\|2\|0 | 2 | 0.67 | 0\|0\|0 | 0.26 | 0.79 | 0.26 | 0.79 | 0.26 | −3.21 | 6.67 | 0.47 |
| Q9P2R3 | ANKFY1 | 0\|0\|2 | 2 | 0.67 | 0\|0\|0 | 0.26 | 0.79 | 0.26 | 0.79 | 0.26 | −3.21 | 6.67 | 0.47 |
| Q9H6R4-4 | NOL6 | 0\|2\|0 | 2 | 0.67 | 0\|0\|0 | 0.26 | 0.79 | 0.26 | 0.79 | 0.26 | −3.21 | 6.67 | 0.47 |
| Q9UMX1 | SUFU | 0\|2\|0 | 2 | 0.67 | 0\|0\|0 | 0.26 | 0.79 | 0.26 | 0.79 | 0.26 | −3.21 | 6.67 | 0.47 |
| Q8N9M1-3 | C19ORF47 | 2\|0\|0 | 2 | 0.67 | 0\|0\|0 | 0.26 | 0.79 | 0.26 | 0.79 | 0.26 | −3.21 | 6.67 | 0.47 |
| Q8NEY1-5 | NAV1 | 0\|2\|0 | 2 | 0.67 | 0\|0\|0 | 0.26 | 0.79 | 0.26 | 0.79 | 0.26 | −3.21 | 6.67 | 0.47 |
| O15294-3 | OGT | 2\|0\|0 | 2 | 0.67 | 0\|0\|0 | 0.26 | 0.79 | 0.26 | 0.79 | 0.26 | −3.21 | 6.67 | 0.47 |
| Q9P0W2-2 | HMG20B | 0\|2\|0 | 2 | 0.67 | 0\|0\|0 | 0.26 | 0.79 | 0.26 | 0.79 | 0.26 | −3.21 | 6.67 | 0.47 |
| Q9NVH1-3 | DNAIC11 | 0\|0\|2 | 2 | 0.67 | 0\|0\|0 | 0.26 | 0.79 | 0.26 | 0.79 | 0.26 | −3.21 | 6.67 | 0.47 |
| Q96A35 | MRPL24 | 2\|0\|0 | 2 | 0.67 | 0\|0\|0 | 0.26 | 0.79 | 0.26 | 0.79 | 0.26 | −3.21 | 6.67 | 0.47 |
| Q9BT92 | TCHP | 2\|0\|0 | 2 | 0.67 | 0\|0\|0 | 0.26 | 0.79 | 0.26 | 0.79 | 0.26 | −3.21 | 6.67 | 0.47 |
| Q7L0Y3 | TRMT10C | 0\|2\|0 | 2 | 0.67 | 0\|0\|0 | 0.26 | 0.79 | 0.26 | 0.79 | 0.26 | −3.21 | 6.67 | 0.47 |
| Q16881-5 | TXNRD1 | 2\|0\|0 | 2 | 0.67 | 0\|0\|0 | 0.26 | 0.79 | 0.26 | 0.79 | 0.26 | −3.21 | 6.67 | 0.47 |
| Q86U44 | METTL3 | 0\|0\|2 | 2 | 0.67 | 0\|0\|0 | 0.26 | 0.79 | 0.26 | 0.79 | 0.26 | −3.21 | 6.67 | 0.47 |
| Q9H479 | FN3K | 0\|2\|0 | 2 | 0.67 | 0\|0\|0 | 0.26 | 0.79 | 0.26 | 0.79 | 0.26 | −3.21 | 6.67 | 0.47 |
| Q9NUL3-4 | STAU2 | 0\|0\|2 | 2 | 0.67 | 0\|0\|0 | 0.26 | 0.79 | 0.26 | 0.79 | 0.26 | −3.21 | 6.67 | 0.47 |
| Q99805 | M9SF2 | 0\|2\|0 | 2 | 0.67 | 0\|0\|0 | 0.26 | 0.79 | 0.26 | 0.79 | 0.26 | −3.21 | 6.67 | 0.47 |
| Q9UM00-1 | TMCO1 | 0\|2\|0 | 2 | 0.67 | 0\|0\|0 | 0.26 | 0.79 | 0.26 | 0.79 | 0.26 | −3.21 | 6.67 | 0.47 |
| Q9NWY4 | HPF1 | 0\|2\|0 | 2 | 0.67 | 0\|0\|0 | 0.26 | 0.79 | 0.26 | 0.79 | 0.26 | −3.21 | 6.67 | 0.47 |
| P55010 | EIFS | 0\|0\|2 | 2 | 0.67 | 0\|0\|0 | 0.26 | 0.79 | 0.26 | 0.79 | 0.26 | −3.21 | 6.67 | 0.47 |
| P29966 | MARCKS | 0\|2\|0 | 2 | 0.67 | 0\|0\|0 | 0.26 | 0.79 | 0.26 | 0.79 | 0.26 | −3.21 | 6.67 | 0.47 |
| Q96S44 | TP53RK | 2\|0\|0 | 2 | 0.67 | 0\|0\|0 | 0.26 | 0.79 | 0.26 | 0.79 | 0.26 | −3.21 | 6.67 | 0.47 |
| Q12933-4 | TRAF2 | 0\|0\|2 | 2 | 0.67 | 0\|0\|0 | 0.26 | 0.79 | 0.26 | 0.79 | 0.26 | −3.21 | 6.67 | 0.47 |
| Q96AG3-3 | SLC25A46 | 0\|2\|0 | 2 | 0.67 | 0\|0\|0 | 0.26 | 0.79 | 0.26 | 0.79 | 0.26 | −3.21 | 6.67 | 0.47 |
| O75794 | CDC123 | 2\|0\|0 | 2 | 0.67 | 0\|0\|0 | 0.26 | 0.79 | 0.26 | 0.79 | 0.26 | −3.21 | 6.67 | 0.47 |
| Q9NP81 | SARS2 | 0\|2\|0 | 2 | 0.67 | 0\|0\|0 | 0.26 | 0.79 | 0.26 | 0.79 | 0.26 | −3.21 | 6.67 | 0.47 |
| Q96IR7 | HPDL | 2\|0\|0 | 2 | 0.67 | 0\|0\|0 | 0.26 | 0.79 | 0.26 | 0.79 | 0.26 | −3.21 | 6.67 | 0.47 |
| P53814-5 | SMTN | 0\|0\|2 | 2 | 0.67 | 0\|0\|0 | 0.26 | 0.79 | 0.26 | 0.79 | 0.26 | −3.21 | 6.67 | 0.47 |
| Q8IWA4 | MFN1 | 0\|2\|0 | 2 | 0.67 | 0\|0\|0 | 0.26 | 0.79 | 0.26 | 0.79 | 0.26 | −3.21 | 6.67 | 0.47 |
| Q9H992-2 | 7-Mar | 0\|0\|2 | 2 | 0.67 | 0\|0\|0 | 0.26 | 0.79 | 0.26 | 0.79 | 0.26 | −3.21 | 6.67 | 0.47 |
| O43314-2 | PPIP5K2 | 0\|2\|0 | 2 | 0.67 | 0\|0\|0 | 0.26 | 0.79 | 0.26 | 0.79 | 0.26 | −3.21 | 6.67 | 0.47 |
| P19838 | NFKB1 | 2\|0\|0 | 2 | 0.67 | 0\|0\|0 | 0.26 | 0.79 | 0.26 | 0.79 | 0.26 | −3.21 | 6.67 | 0.47 |
| P51570 | GALK1 | 2\|0\|0 | 2 | 0.67 | 0\|0\|0 | 0.26 | 0.79 | 0.26 | 0.79 | 0.26 | −3.21 | 6.67 | 0.47 |
| Q9NP97 | DYNLRB1 | 0\|2\|0 | 2 | 0.67 | 0\|0\|0 | 0.26 | 0.79 | 0.26 | 0.79 | 0.26 | −3.21 | 6.67 | 0.47 |
| Q15418-4 | RPS6KA1 | 2\|0\|0 | 2 | 0.67 | 0\|0\|0 | 0.26 | 0.79 | 0.26 | 0.79 | 0.26 | −3.21 | 6.67 | 0.47 |
| P57737-3 | CORO7 | 0\|2\|0 | 2 | 0.67 | 0\|0\|0 | 0.26 | 0.79 | 0.26 | 0.79 | 0.26 | −3.21 | 6.67 | 0.47 |
| Q9Y222-2 | MTO1 | 2\|0\|0 | 2 | 0.67 | 0\|0\|0 | 0.26 | 0.79 | 0.26 | 0.79 | 0.26 | −3.21 | 6.67 | 0.47 |
| Q9H936 | SLC25A22 | 0\|0\|2 | 2 | 0.67 | 0\|0\|0 | 0.26 | 0.79 | 0.26 | 0.79 | 0.26 | −3.21 | 6.67 | 0.47 |
| Q86YZ3 | HRNR | 0\|0\|2 | 2 | 0.67 | 0\|0\|0 | 0.26 | 0.79 | 0.26 | 0.79 | 0.26 | −3.21 | 6.67 | 0.47 |
| Q8WVX9 | FAR1 | 2\|0\|0 | 2 | 0.67 | 0\|0\|0 | 0.26 | 0.79 | 0.26 | 0.79 | 0.26 | −3.21 | 6.67 | 0.47 |
| P21281 | ATP6V1B2 | 0\|2\|0 | 2 | 0.67 | 0\|0\|0 | 0.26 | 0.79 | 0.26 | 0.79 | 0.26 | −3.21 | 6.67 | 0.47 |
| O95983-2 | MBD3 | 0\|0\|2 | 2 | 0.67 | 0\|0\|0 | 0.26 | 0.79 | 0.26 | 0.79 | 0.26 | −3.21 | 6.67 | 0.47 |
| Q9BQL6 | FERMT1 | 0\|0\|2 | 2 | 0.67 | 0\|0\|0 | 0.26 | 0.79 | 0.26 | 0.79 | 0.26 | −3.21 | 6.67 | 0.47 |
| Q9P2Y4 | ZNF219 | 0\|0\|2 | 2 | 0.67 | 0\|0\|0 | 0.26 | 0.79 | 0.26 | 0.79 | 0.26 | −3.21 | 6.67 | 0.47 |
| P18074-2 | ERCC2 | 2\|0\|0 | 2 | 0.67 | 0\|0\|0 | 0.26 | 0.79 | 0.26 | 0.79 | 0.26 | −3.21 | 6.67 | 0.47 |
| Q9NZL9-4 | MAT2B | 2\|0\|0 | 2 | 0.67 | 0\|0\|0 | 0.26 | 0.79 | 0.26 | 0.79 | 0.26 | −3.21 | 6.67 | 0.47 |
| Q9BUB7 | TMEM70 | 0\|2\|0 | 2 | 0.67 | 0\|0\|0 | 0.26 | 0.79 | 0.26 | 0.79 | 0.26 | −3.21 | 6.67 | 0.47 |
| Q53EP0 | FNDC3B | 0\|2\|0 | 2 | 0.67 | 0\|0\|0 | 0.26 | 0.79 | 0.26 | 0.79 | 0.26 | −3.21 | 6.67 | 0.47 |
| O75879 | GATB | 2\|0\|0 | 3 | 0.67 | 0\|0\|0 | 0.26 | 0.79 | 0.26 | 0.79 | 0.26 | −3.21 | 6.67 | 0.47 |
| O75616 | ERAL1 | 2\|0\|0 | 2 | 0.67 | 0\|0\|0 | 0.26 | 0.79 | 0.26 | 0.79 | 0.26 | −3.21 | 6.67 | 0.47 |
| Q05932-3 | FPGS | 0\|2\|0 | 2 | 0.67 | 0\|0\|0 | 0.26 | 0.79 | 0.26 | 0.79 | 0.26 | −3.21 | 6.67 | 0.47 |
| P49642 | PRIM1 | 0\|0\|2 | 2 | 0.67 | 0\|0\|0 | 0.26 | 0.79 | 0.26 | 0.79 | 0.26 | −3.21 | 6.67 | 0.47 |
| P31350 | RRM2 | 0\|2\|0 | 2 | 0.67 | 0\|0\|0 | 0.26 | 0.79 | 0.26 | 0.79 | 0.26 | −3.21 | 6.67 | 0.47 |
| Q2M218-2 | AAK1 | 2\|0\|0 | 2 | 0.67 | 0\|0\|0 | 0.26 | 0.79 | 0.26 | 0.79 | 0.26 | −3.21 | 6.67 | 0.47 |
| Q13371-2 | PDCL | 0\|0\|2 | 2 | 0.67 | 0\|0\|0 | 0.26 | 0.79 | 0.26 | 0.79 | 0.26 | −3.21 | 6.67 | 0.47 |
| Q92888-2 | ARHGEF1 | 0\|2\|0 | 2 | 0.67 | 0\|0\|0 | 0.26 | 0.79 | 0.26 | 0.79 | 0.26 | −3.21 | 6.67 | 0.47 |
| P50747 | HLCS | 2\|0\|0 | 2 | 0.67 | 0\|0\|0 | 0.26 | 0.79 | 0.26 | 0.79 | 0.26 | −3.21 | 6.67 | 0.47 |
| Q7Z3K3-5 | POGZ | 0\|2\|0 | 2 | 0.67 | 0\|0\|0 | 0.26 | 0.79 | 0.26 | 0.79 | 0.26 | −3.21 | 6.67 | 0.47 |
| A6NNY8 | USP27X | 0\|0\|2 | 2 | 0.67 | 0\|0\|0 | 0.26 | 0.79 | 0.26 | 0.79 | 0.26 | −3.21 | 6.67 | 0.47 |
| Q9UKY7-2 | CDV3 | 0\|0\|2 | 2 | 0.67 | 0\|0\|0 | 0.26 | 0.79 | 0.26 | 0.79 | 0.26 | −3.21 | 6.67 | 0.47 |
| P05026-2 | ATP1B1 | 2\|0\|0 | 3 | 0.67 | 0\|0\|0 | 0.26 | 0.79 | 0.26 | 0.79 | 0.26 | −3.21 | 6.67 | 0.47 |
| Q96A49 | SYAP1 | 0\|2\|0 | 2 | 0.67 | 0\|0\|0 | 0.26 | 0.79 | 0.26 | 0.79 | 0.26 | −3.21 | 6.67 | 0.47 |
| Q6IAN0 | DHRS7B | 0\|2\|0 | 2 | 0.67 | 0\|0\|0 | 0.26 | 0.79 | 0.26 | 0.79 | 0.26 | −3.21 | 6.67 | 0.47 |
| P28074 | PSMBS | 2\|0\|0 | 2 | 0.67 | 0\|0\|0 | 0.26 | 0.79 | 0.26 | 0.79 | 0.26 | −3.21 | 6.67 | 0.47 |
| P51114-3 | FXR1 | 0\|2\|0 | 2 | 0.67 | 0\|0\|0 | 0.26 | 0.79 | 0.26 | 0.79 | 0.26 | −3.21 | 6.67 | 0.47 |
| P39019 | RPS19 | 0\|0\|2 | 2 | 0.67 | 0\|0\|0 | 0.26 | 0.79 | 0.26 | 0.79 | 0.26 | −3.21 | 6.67 | 0.47 |
| P60228 | EIF3E | 0\|0\|2 | 2 | 0.67 | 0\|0\|0 | 0.26 | 0.79 | 0.26 | 0.79 | 0.26 | −3.21 | 6.67 | 0.47 |
| Q9BRZ2 | TRIM56 | 0\|0\|2 | 2 | 0.67 | 0\|0\|0 | 0.26 | 0.79 | 0.26 | 0.79 | 0.26 | −3.21 | 6.67 | 0.47 |
| Q9NWU5-1 | MRPL22 | 0\|2\|0 | 2 | 0.67 | 0\|0\|0 | 0.26 | 0.79 | 0.26 | 0.79 | 0.26 | −3.21 | 6.67 | 0.47 |
| Q8WUB8-3 | PHF10 | 0\|0\|2 | 2 | 0.67 | 0\|0\|0 | 0.26 | 0.79 | 0.26 | 0.79 | 0.26 | −3.21 | 6.67 | 0.47 |
| Q9Y217 | PIKFYVE | 2\|0\|0 | 2 | 0.67 | 0\|0\|0 | 0.26 | 0.79 | 0.26 | 0.79 | 0.26 | −3.21 | 6.67 | 0.47 |
| P21266 | GSTM3 | 0\|0\|2 | 2 | 0.67 | 0\|0\|0 | 0.26 | 0.79 | 0.26 | 0.79 | 0.26 | −3.21 | 6.67 | 0.47 |
| O60884 | DNAJA2 | 2\|4\|3 | 9 | 3 | 0\|0\|2 | 0.25 | 0.41 | 0.25 | 0.41 | 0.25 | −2.03 | 4.5 | 0.62 |
| O95373 | IPO7 | 3\|4\|2 | 9 | 3 | 0\|0\|2 | 0.25 | 0.41 | 0.25 | 0.41 | 0.25 | −2.03 | 4.5 | 0.62 |
| P38606-2 | ATP6VIA | 4\|3\|2 | 9 | 3 | 2\|0\|0 | 0.25 | 0.41 | 0.25 | 0.41 | 0.25 | −2.03 | 4.5 | 0.62 |
| Q9P0U3-2 | SENP1 | 0\|5\|2 | 7 | 2.33 | 2\|0\|0 | 0.24 | 0.59 | 0.24 | 0.59 | 0.24 | −5.38 | 3.5 | 0.62 |
| Q15650 | TRIP4 | 0\|4\|3 | 7 | 2.33 | 2\|0\|0 | 0.22 | 0.41 | 0.22 | 0.41 | 0.22 | −5.38 | 3.5 | 0.62 |
| Q9HB19 | PLEKHA2 | 3\|5\|3 | 11 | 3.67 | 0\|0\|3 | 0.21 | 0.35 | 0.21 | 0.35 | 0.21 | −1.84 | 3.67 | 0.62 |
| Q8N6T3 | ARFGAP1 | 2\|3\|10 | 15 | 5 | 0\|3\|4 | 0.21 | 0.63 | 0.21 | 0.63 | 0.21 | −7.93 | 2.14 | 0.62 |
| Q6IQ22 | RAB12 | 4\|5\|5 | 14 | 4.67 | 4\|0\|0 | 0.21 | 0.25 | 0.21 | 0.25 | 0.21 | −1.76 | 3.5 | 0.62 |
| Q6N021 | TET2 | 0\|7\|2 | 9 | 3 | 0\|3\|0 | 0.21 | 0.58 | 0.21 | 0.58 | 0.21 | −5.8 | 3 | 0.62 |
| O95604 | HLA-C | 4\|3\|4 | 11 | 3.67 | 0\|3\|0 | 0.21 | 0.25 | 0.21 | 0.25 | 0.21 | −1.84 | 3.67 | 0.62 |
| P25789 | PSMA4 | 3\|3\|2 | 8 | 2.67 | 0\|0\|2 | 0.2 | 0.24 | 0.2 | 0.24 | 0.2 | −2.03 | 4 | 0.63 |
| P55735-2 | SEC13 | 5\|0\|0 | 5 | 1.67 | 0\|0\|2 | 0.2 | 0.59 | 0.2 | 0.59 | 0.2 | −5.38 | 2.5 | 0.62 |
| Q04206-2 | RELA | 7\|3\|2 | 12 | 4 | 3\|2\|0 | 0.19 | 0.56 | 0.19 | 0.56 | 0.19 | −5.66 | 2.4 | 0.63 |
| Q02413 | DSG1 | 4\|2\|0 | 6 | 2 | 0\|2\|0 | 0.17 | 0.41 | 0.17 | 0.41 | 0.17 | −5.38 | 3 | 0.63 |
| P35222 | CTNNB1 | 4\|0\|2 | 6 | 2 | 0\|2\|0 | 0.17 | 0.41 | 0.17 | 0.41 | 0.17 | −5.38 | 3 | 0.63 |
| Q13330-2 | MTA1 | 016\|5 | 11 | 3.67 | 2\|0\|3 | 0.16 | 0.33 | 0.16 | 0.33 | 0.16 | −9.13 | 2.2 | 0.63 |
| Q9Y2P8 | RCL1 | 2\|3\|2 | 7 | 2.33 | 2\|0\|0 | 0.16 | 0.24 | 0.16 | 0.24 | 0.16 | −2.03 | 3.5 | 0.63 |
| P02452 | COL1A1 | 2\|2\|3 | 7 | 2.33 | 0\|2\|0 | 0.16 | 0.24 | 0.16 | 0.24 | 0.16 | −2.03 | 3.5 | 0.63 |
| Q8NC60 | NOA1 | 0\|5\|0 | 5 | 1.67 | 2\|2\|0 | 0.15 | 0.46 | 0.15 | 0.46 | 0.15 | −7.8 | 1.25 | 0.63 |
| Q8ND04 | SMG8 | 2\|3\|4 | 9 | 3 | 0\|0\|3 | 0.15 | 0.25 | 0.15 | 0.25 | 0.15 | −2.88 | 3 | 0.63 |
| Q9UNL2 | SSR3 | 3\|3\|3 | 9 | 3 | 3\|0\|0 | 0.14 | 0.14 | 0.14 | 0.14 | 0.14 | −1.84 | 3 | 0.63 |
| P50750 | CDK9 | 3\|3\|3 | 9 | 3 | 0\|3\|0 | 0.14 | 0.14 | 0.14 | 0.14 | 0.14 | −1.84 | 3 | 0.63 |
| Q9H9Y6-5 | POLR18 | 3\|5\|3 | 11 | 3.67 | 4\|0\|0 | 0.13 | 0.25 | 0.13 | 0.25 | 0.13 | −2.65 | 2.75 | 0.64 |
| P99999 | CYCS | 2\|2\|2 | 6 | 2 | 0\|2\|0 | 0.12 | 0.12 | 0.12 | 0.12 | 0.12 | −2.03 | 3 | 0.65 |
| Q9H425 | C1ORF198 | 2\|3\|0 | 5 | 1.67 | 0\|0\|2 | 0.12 | 0.24 | 0.12 | 0.24 | 0.12 | −5.38 | 2.5 | 0.64 |
| O60942 | RNGTT | 3\|2\|0 | 5 | 1.67 | 0\|0\|2 | 0.12 | 0.24 | 0.12 | 0.24 | 0.12 | −5.38 | 2.5 | 0.64 |
| Q9UKK3 | PARP4 | 0\|2\|3 | 5 | 1.67 | 0\|0\|2 | 0.12 | 0.24 | 0.12 | 0.24 | 0.1 | −5.38 | 2.5 | 0.64 |
| P78318 | IGBP1 | 2\|2\|2 | 6 | 2 | 0\|0\|2 | 0.12 | 0.12 | 0.12 | 0.12 | 0.12 | −2.03 | 3 | 0.65 |
| Q00653-4 | NFKB2 | 4\|4\|3 | 11 | 3.67 | 0\|4\|0 | 0.12 | 0.15 | 0.12 | 0.15 | 0.12 | −2.65 | 2.75 | 0.64 |
| P08240 | SRPRA | 0\|3\|2 | 5 | 1.67 | 2\|0\|0 | 0.12 | 0.24 | 0.12 | 0.24 | 0.12 | −5.38 | 2.5 | 0.64 |
| Q9C0C9 | UBE20 | 3\|0\|2 | 5 | 1.67 | 0\|2\|0 | 0.12 | 0.24 | 0.12 | 0.24 | 0.12 | −5.38 | 2.5 | 0.64 |
| P49674 | CSNK1E | 2\|2\|2 | 6 | 2 | 0\|2\|0 | 0.1 | 0.12 | 0.12 | 0.12 | 0.12 | −2.03 | 3 | 0.65 |
| Q12905 | ILF2 | 2\|2\|4 | 8 | 2.67 | 0\|3\|0 | 0.12 | 0.25 | 0.12 | 0.25 | 0.12 | −2.88 | 2.67 | 0.64 |
| O60568 | PLOD3 | 3\|0\|2 | 5 | 1.67 | 2\|0\|0 | 0.12 | 0.24 | 0.12 | 0.24 | 0.12 | −5.38 | 2.5 | 0.64 |
| Q86TG7 | PEG10 | 8\|11\|11 | 30 | 10 | 5\|5\|2 | 0.11 | 0.17 | 0.11 | 0.17 | 0.11 | −5.47 | 2.5 | 0.65 |
| Q15031 | LARS2 | 5\|2\|3 | 10 | 3.33 | 4\|0\|0 | 0.11 | 0.25 | 0.11 | 0.25 | 0.11 | −3.87 | 2.5 | 0.65 |
| Q9BYJ9 | YTHDF1 | 2\|4\|0 | 6 | 2 | 3\|0\|0 | 0.1 | 0.25 | 0.1 | 0.25 | 0.1 | −5.8 | 2 | 0.65 |
| P78406 | RAE1 | 0\|5\|3 | 8 | 2.67 | 4\|0\|0 | 0.1 | 0.25 | 0.1 | 0.25 | 0.1 | −7.28 | 2 | 0.65 |
| Q14432 | PDE3A | 7\|9\|2 | 18 | 6 | 2\|3\|4 | 0.1 | 0.28 | 0.1 | 0.28 | 0.1 | −10.42 | 2 | 0.65 |
| Q9UNY4 | TTF2 | 5\|3\|8 | 16 | 5.33 | 3\|4\|0 | 0.1 | 0.28 | 0.1 | 0.28 | 0.1 | −6.41 | 2.29 | 0.65 |
| O00399 | DCTN6 | 2\|0\|2 | 4 | 1.33 | 0\|0\|2 | 0.08 | 0.12 | 0.08 | 0.12 | 0.08 | −5.38 | 2 | 0.67 |
| Q96CS3 | FAF2 | 8\|3\|4 | 15 | 5 | 3\|2\|3 | 0.08 | 0.24 | 0.08 | 0.24 | 0.08 | −7.81 | 1.88 | 0.66 |
| Q15165-3 | PON2 | 2\|0\|2 | 4 | 1.33 | 0\|0\|2 | 0.08 | 0.12 | 0.08 | 0.12 | 0.08 | −5.38 | 2 | 0.67 |
| O8WYA6-4 | CTNNBL1 | 0\|2\|2 | 4 | 1.33 | 0\|0\|2 | 0.08 | 0.12 | 0.08 | 0.12 | 0.08 | −5.38 | 2 | 0.67 |
| Q15776 | ZKSCAN8 | 2\|2\|0 | 4 | 1.33 | 0\|0\|2 | 0.08 | 0.12 | 0.08 | 0.12 | 0.08 | −5.38 | 2 | 0.67 |
| P61604 | HSPE3 | 2\|2\|0 | 4 | 1.33 | 0\|0\|2 | 0.08 | 0.12 | 0.08 | 0.12 | 0.08 | −5.38 | 2 | 0.67 |
| Q8TC12 | STT3B | 0\|3\|0 | 3 | 1 | 0\|0\|2 | 0.08 | 0.24 | 0.08 | 0.24 | 0.08 | −5.38 | 1.5 | 0.66 |
| P62633-2 | CNBP | 0\|0\|4 | 4 | 1.33 | 3\|0\|0 | 0.08 | 0.25 | 0.08 | 0.25 | 0.08 | −5.8 | 1.33 | 0.66 |
| Q9C0D5-2 | TANC1 | 0\|0\|3 | 3 | 1 | 0\|0\|2 | 0.08 | 0.24 | 0.08 | 0.24 | 0.08 | −5.38 | 1.5 | 0.66 |
| O14757-2 | CHEK1 | 2\|0\|2 | 4 | 1.33 | 0\|0\|2 | 0.08 | 0.12 | 0.08 | 0.12 | 0.08 | −5.38 | 2 | 0.67 |
| Q61A86-2 | ELP2 | 0\|2\|2 | 4 | 1.33 | 0\|0\|2 | 0.08 | 0.12 | 0.08 | 0.12 | 0.08 | −5.38 | 2 | 0.67 |
| Q04446 | GBE1 | 0\|2\|2 | 4 | 1.33 | 0\|2\|0 | 0.08 | 0.12 | 0.08 | 0.12 | 0.08 | −5.38 | 2 | 0.67 |
| O00410-2 | IPO5 | 2\|2\|0 | 4 | 1.33 | 0\|2\|0 | 0.08 | 0.12 | 0.08 | 0.12 | 0.08 | −5.38 | 2 | 0.67 |
| P51809-3 | VAMP7 | 2\|0\|2 | 4 | 1.33 | 0\|2\|0 | 0.08 | 0.12 | 0.08 | 0.12 | 0.08 | −5.38 | 2 | 0.67 |
| Q9GZT9-2 | EGLN1 | 0\|3\|0 | 3 | 1 | 0\|0\|2 | 0.08 | 0.24 | 0.08 | 0.24 | 0.08 | −5.38 | 1.5 | 0.66 |
| O60573-2 | EIF4E2 | 0\|0\|3 | 3 | 1 | 2\|0\|0 | 0.08 | 0.24 | 0.08 | 0.24 | 0.08 | −5.38 | 1.5 | 0.66 |
| P49840 | GSK3A | 2\|3\|2 | 7 | 2.33 | 0\|0\|3 | 0.08 | 0.14 | 0.08 | 0.14 | 0.08 | −2.88 | 2.33 | 0.66 |
| P57678 | GEMIN4 | 2\|2\|3 | 7 | 2.33 | 0\|0\|3 | 0.08 | 0.14 | 0.08 | 0.14 | 0.08 | −2.88 | 2.33 | 0.66 |
| Q14847 | LASP1 | 4\|7\|5 | 16 | 5.33 | 2\|2\|3 | 0.08 | 0.21 | 0.08 | 0.21 | 0.08 | −5.22 | 2.29 | 0.67 |
| P49589-3 | CARS | 2\|2\|0 | 4 | 1.33 | 2\|0\|0 | 0.08 | 0.12 | 0.08 | 0.12 | 0.08 | −5.38 | 2 | 0.67 |
| Q9NUL7 | DDX28 | 2\|0\|2 | 4 | 1.33 | 2\|0\|0 | 0.08 | 0.12 | 0.08 | 0.12 | 0.08 | −5.38 | 2 | 0.67 |
| O99575 | POP1 | 0\|2\|2 | 4 | 1.33 | 2\|0\|0 | 0.08 | 0.12 | 0.08 | 0.12 | 0.08 | −5.38 | 2 | 0.67 |
| O75319 | DUSP11 | 0\|3\|0 | 3 | 1 | 0\|2\|0 | 0.08 | 0.24 | 0.08 | 0.24 | 0.08 | −5.38 | 1.5 | 0.66 |
| Q9H6T3-2 | RPAP3 | 3\|0\|0 | 3 | 1 | 2\|0\|0 | 0.08 | 0.24 | 0.08 | 0.24 | 0.08 | −5.38 | 1.5 | 0.66 |
| O43847 | NRDC | 0\|0\|5 | 5 | 1.67 | 0\|4\|0 | 0.08 | 0.25 | 0.08 | 0.25 | 0.08 | −7.28 | 1.25 | 0.66 |
| P08134 | RHOC | 2\|0\|2 | 4 | 1.33 | 0\|0\|2 | 0.08 | 0.12 | 0.08 | 0.12 | 0.08 | −5.38 | 2 | 0.67 |
| Q9UK76 | JPT1 | 0\|3\|0 | 3 | 1 | 2\|0\|0 | 0.08 | 0.24 | 0.08 | 0.24 | 0.08 | −5.38 | 1.5 | 0.66 |
| P86791 | CCZ1 | 2\|2\|0 | 4 | 1.33 | 2\|0\|0 | 0.08 | 0.12 | 0.08 | 0.12 | 0.08 | −5.38 | 2 | 0.67 |
| A8CG34 | POM121C | 2\|2\|0 | 4 | 1.33 | 0\|0\|2 | 0.08 | 0.12 | 0.08 | 0.12 | 0.08 | −5.38 | 2 | 0.67 |
| Q9BZV1-2 | UBXN6 | 0\|2\|2 | 4 | 1.33 | 0\|0\|2 | 0.08 | 0.12 | 0.08 | 0.12 | 0.08 | −5.38 | 2 | 0.67 |
| O75530-3 | EED | 0\|2\|2 | 4 | 1.33 | 2\|0\|0 | 0.08 | 0.12 | 0.08 | 0.12 | 0.08 | −5.38 | 2 | 0.67 |
| Q8NEZS | FBXO22 | 2\|0\|2 | 4 | 1.33 | 2\|0\|0 | 0.08 | 0.12 | 0.08 | 0.12 | 0.08 | −5.38 | 2 | 0.67 |
| Q93008-1 | USP9X | 2\|2\|0 | 4 | 1.33 | 0\|0\|2 | 0.08 | 0.12 | 0.08 | 0.12 | 0.08 | −5.38 | 2 | 0.67 |
| P51692 | STAT5B | 2\|2\|0 | 4 | 1.33 | 0\|0\|2 | 0.08 | 0.12 | 0.08 | 0.12 | 0.08 | −5.38 | 2 | 0.67 |
| O95861-4 | BPNT1 | 2\|2\|0 | 4 | 1.33 | 2\|0\|0 | 0.08 | 0.12 | 0.08 | 0.12 | 0.08 | −5.38 | 2 | 0.67 |
| Q9H583 | HEATR1 | 0\|0\|3 | 3 | 1 | 0\|0\|2 | 0.08 | 0.24 | 0.08 | 0.24 | 0.08 | −5.38 | 1.5 | 0.66 |
| Q9BVS4 | RIOK2 | 0\|2\|2 | 0 | 1.33 | 0\|2\|0 | 0.08 | 0.12 | 0.08 | 0.12 | 0.08 | −5.38 | 2 | 0.67 |
| Q13283 | G3BP1 | 7\|5\|5 | 17 | 5.67 | 2\|3\|2 | 0.08 | 0.21 | 0.08 | 0.21 | 0.08 | −3.87 | 2.43 | 0.66 |
| O00159-2 | MYO1C | 0\|3\|0 | 3 | 1 | 2\|0\|0 | 0.08 | 0.24 | 0.08 | 0.24 | 0.08 | −5.38 | 1.5 | 0.66 |
| O14744-4 | PRMTS | 0\|3\|0 | 3 | 3 | 2\|0\|0 | 0.08 | 0.24 | 0.08 | 0.24 | 0.08 | −5.38 | 1.5 | 0.66 |
| Q9H4A4 | RNPEP | 2\|2\|0 | 4 | 1.33 | 0\|0\|2 | 0.08 | 0.12 | 0.08 | 0.12 | 0.08 | −5.38 | 2 | 0.67 |
| Q8NEC7-3 | GSTCD | 2\|0\|2 | 4 | 1.33 | 2\|0\|0 | 0.08 | 0.12 | 0.08 | 0.12 | 0.08 | −5.38 | 2 | 0.67 |
| P00492 | HPRT1 | 2\|0\|2 | 4 | 1.33 | 2\|0\|0 | 0.08 | 0.12 | 0.08 | 0.12 | 0.08 | −5.38 | 2 | 0.67 |
| P43487-2 | RANBP1 | 2\|0\|2 | 4 | 1.33 | 0\|2\|0 | 0.08 | 0.12 | 0.08 | 0.12 | 0.08 | −5.38 | 2 | 0.67 |
| Q9Y2X3 | NOP58 | 2\|2\|0 | 0 | 1.33 | 0\|0\|2 | 0.08 | 0.12 | 0.08 | 0.12 | 0.08 | −5.38 | 2 | 0.67 |
| A1L0T0 | ILVBL | 3\|4\|3 | 10 | 3.33 | 2\|0\|2 | 0.08 | 0.15 | 0.08 | 0.15 | 0.08 | −3.21 | 2.5 | 0.67 |
| P27105 | STOM | 3\|0\|0 | 3 | 1 | 0\|0\|2 | 0.08 | 0.24 | 0.08 | 0.24 | 0.08 | −5.38 | 1.5 | 0.66 |
| O00178 | GTPBP1 | 4\|5\|3 | 12 | 4 | 2\|3\|0 | 0.07 | 0.14 | 0.07 | 0.14 | 0.07 | −4.25 | 2.4 | 0.7 |
| P30086 | PEBP1 | 3\|5\|4 | 12 | 4 | 0\|2\|3 | 0.07 | 0.14 | 0.07 | 0.14 | 0.07 | −4.25 | 2.4 | 0.7 |
| Q9H0S4 | DDX47 | 2\|3\|0 | 5 | 1.67 | 0\|3\|0 | 0.06 | 0.14 | 0.06 | 0.14 | 0.06 | −5.8 | 1.67 | 0.7 |
| Q9NVP1 | DDX18 | 2\|0\|3 | 5 | 1.67 | 0\|0\|3 | 0.06 | 0.14 | 0.06 | 0.14 | 0.06 | −5.8 | 1.67 | 0.7 |
| Q9Y262 | EIF3L | 2\|3\|0 | 5 | 1.67 | 0\|3\|0 | 0.06 | 0.14 | 0.06 | 0.14 | 0.06 | −5.8 | 1.67 | 0.7 |
| Q5GLZ8-6 | HERC4 | 2\|4\|2 | 8 | 2.67 | 0\|2\|2 | 0.06 | 0.15 | 0.06 | 0.15 | 0.06 | −4.67 | 2 | 0.7 |
| Q14558 | PRPSAP1 | 2\|4\|2 | 8 | 2.67 | 2\|0\|2 | 0.06 | 0.15 | 0.06 | 0.15 | 0.06 | −4.67 | 2 | 0.7 |
| Q12792-4 | TWF1 | 3\|0\|2 | 5 | 1.67 | 3\|0\|0 | 0.06 | 0.14 | 0.06 | 0.14 | 0.06 | −5.8 | 1.67 | 0.7 |
| Q8NFQ8 | TORLAIP2 | 2\|2\|2 | 6 | 2 | 0\|3\|0 | 0.05 | 0.05 | 0.05 | 0.05 | 0.05 | −2.88 | 2 | 0.71 |
| P61619 | SEC61A1 | 5\|3\|2 | 10 | 3.33 | 0\|2\|3 | 0.05 | 0.14 | 0.05 | 0.14 | 0.05 | −5.66 | 2 | 0.71 |
| Q5SRE5 | NUP188 | 4\|4\|4 | 12 | 4 | 0\|2\|3 | 0.05 | 0.05 | 0.05 | 0.05 | 0.05 | −2.97 | 2.4 | 0.71 |
| O14976-2 | GAK | 4\|4\|4 | 12 | 4 | 3\|0\|2 | 0.05 | 0.05 | 0.05 | 0.05 | 0.05 | −2.97 | 2.4 | 0.71 |
| Q9H6S0 | YTHDC2 | 4\|0\|0 | 4 | 1.33 | 2\|0\|2 | 0.05 | 0.15 | 0.05 | 0.15 | 0.05 | −7.8 | 1 | 0.71 |
| P01116-2 | KRAS | 3\|3\|2 | 8 | 2.67 | 0\|0\|4 | 0.05 | 0.07 | 0.05 | 0.07 | 0.05 | −3.87 | 2 | 0.71 |
| P61964 | WDRS | 2\|4\|0 | 6 | 2 | 0\|2\|2 | 0.05 | 0.15 | 0.05 | 0.15 | 0.05 | −7.8 | 1.5 | 0.71 |
| P82930 | MRPS34 | 0\|0\|3 | 3 | 3 | 3\|0\|0 | 0.05 | 0.14 | 0.05 | 0.14 | 0.05 | −5.8 | 1 | 0.72 |
| O75330-2 | HMMR | 5\|0\|0 | 5 | 1.67 | 2\|3\|0 | 0.05 | 0.14 | 0.05 | 0.14 | 0.05 | −9.13 | 1 | 0.72 |
| O95376 | ARIH2 | 0\|3\|0 | 3 | 1 | 3\|0\|0 | 0.05 | 0.14 | 0.05 | 0.14 | 0.05 | −5.8 | 1 | 0.72 |
| Q9Y3B2 | EXOSC1 | 5\|2\|2 | 9 | 3 | 3\|2\|0 | 0.05 | 0.14 | 0.05 | 0.14 | 0.05 | −5.66 | 1.8 | 0.71 |
| Q9NQH7-2 | XPNPEP3 | 0\|0\|3 | 3 | 1 | 0\|0\|3 | 0.05 | 0.14 | 0.05 | 0.14 | 0.05 | −5.8 | 1 | 0.72 |
| Q969N2-4 | PIGT | 2\|4\|0 | 6 | 2 | 0\|2\|2 | 0.05 | 0.15 | 0.05 | 0.15 | 0.05 | −7.8 | 1.5 | 0.71 |
| Q9Y5K5-2 | UCHLS | 3\|0\|0 | 3 | 3 | 3\|0\|0 | 0.05 | 0.14 | 0.05 | 0.14 | 0.05 | −5.8 | 1 | 0.72 |
| O99570 | PIK3R4 | 0\|2\|4 | 6 | 2 | 0\|2\|2 | 0.05 | 0.15 | 0.05 | 0.15 | 0.05 | −7.8 | 1.5 | 0.71 |
| Q9NWT8 | AURKAIP1 | 3\|0\|0 | 3 | 3 | 3\|0\|0 | 0.05 | 0.14 | 0.05 | 0.14 | 0.05 | −5.8 | 1 | 0.72 |
| Q92598-2 | HSPH1 | 5\|2\|2 | 9 | 3 | 0\|5\|0 | 0.05 | 0.15 | 0.05 | 0.15 | 0.05 | −4.96 | 1.8 | 0.71 |
| Q50862 | FLG2 | 4\|0\|0 | 4 | 1.33 | 0\|4\|0 | 0.05 | 0.15 | 0.05 | 0.15 | 0.05 | −7.28 | 1 | 0.71 |
| Q92536 | SLC7A6 | 4\|0\|0 | 4 | 1.33 | 0\|0\|4 | 0.05 | 0.15 | 0.05 | 0.15 | 0.05 | −7.28 | 1 | 0.71 |
| Q9NR50-3 | EIF283 | 5\|4\|10 | 19 | 6.33 | 4\|3\|4 | 0.05 | 0.14 | 0.05 | 0.14 | 0.05 | −9.88 | 1.73 | 0.72 |
| P49023-2 | PXN | 0\|0\|2 | 2 | 0.67 | 0\|0\|2 | 0.04 | 0.12 | 0.04 | 0.12 | 0.04 | −5.38 | 1 | 0.72 |
| Q96HA1 | POM121 | 2\|2\|0 | 4 | 1.33 | 0\|0\|3 | 0.04 | 0.05 | 0.04 | 0.05 | 0.04 | −5.8 | 1.33 | 0.75 |
| O75165 | DNAIC13 | 0\|2\|0 | 2 | 0.67 | 0\|2\|0 | 0.04 | 0.12 | 0.04 | 0.12 | 0.04 | −5.38 | 3 | 0.72 |
| P61163 | ACTRIA | 2\|0\|0 | 2 | 0.6 | 0\|0\|2 | 0.04 | 0.12 | 0.04 | 0.12 | 0.04 | −5.38 | 1 | 0.72 |
| Q9Y2T2 | AP3M1 | 0\|2\|0 | 2 | 0.67 | 0\|0\|2 | 0.04 | 0.12 | 0.04 | 0.12 | 0.04 | −5.38 | 3 | 0.72 |
| O15460-2 | P4HA2 | 0\|2\|2 | 4 | 1.33 | 0\|3\|0 | 0.04 | 0.05 | 0.04 | 0.05 | 0.04 | −5.8 | 1.33 | 0.75 |
| Q86TB9-4 | PATL1 | 2\|0\|2 | 4 | 1.33 | 0\|0\|3 | 0.04 | 0.05 | 0.04 | 0.05 | 0.04 | −5.8 | 1.33 | 0.75 |
| Q8IZT6-2 | ASPM | 0\|2\|0 | 2 | 0.67 | 0\|0\|2 | 0.04 | 0.12 | 0.04 | 0.12 | 0.04 | −5.38 | 3 | 0.72 |
| Q7Z2E3-7 | APTX | 2\|2\|0 | 4 | 1.33 | 3\|0\|0 | 0.04 | 0.05 | 0.04 | 0.05 | 0.04 | −5.8 | 1.33 | 0.75 |
| Q16643 | DBN1 | 2\|0\|0 | 2 | 0.67 | 2\|0\|0 | 0.04 | 0.12 | 0.04 | 0.12 | 0.04 | −5.38 | 1 | 0.72 |
| Q92879-2 | CELF1 | 0\|2\|2 | 4 | 1.33 | 3\|0\|0 | 0.04 | 0.05 | 0.04 | 0.05 | 0.04 | −5.8 | 1.33 | 0.75 |
| QST1M5 | FKBP15 | 0\|2\|0 | 2 | 0.67 | 0\|2\|0 | 0.04 | 0.12 | 0.04 | 0.12 | 0.04 | −5.38 | 1 | 0.72 |
| O15504 | NUPL2 | 0\|2\|0 | 2 | 0.67 | 2\|0\|0 | 0.04 | 0.12 | 0.04 | 0.12 | 0.04 | −5.38 | 1 | 0.72 |
| A0FGR8-2 | ESYT2 | 0\|2\|0 | 2 | 0.67 | 0\|0\|2 | 0.04 | 0.12 | 0.04 | 0.12 | 0.04 | −5.38 | 3 | 0.72 |
| O14530-2 | TXNDC9 | 0\|0\|2 | 2 | 0.67 | 0\|0\|2 | 0.04 | 0.12 | 0.04 | 0.12 | 0.04 | −5.38 | 1 | 0.72 |
| Q13625-2 | TP53BP2 | 0\|2\|0 | 2 | 0.67 | 2\|0\|0 | 0.04 | 0.12 | 0.04 | 0.12 | 0.04 | −5.38 | 3 | 0.72 |
| P25786 | PSMA1 | 5\|4\|5 | 14 | 4.67 | 3\|3\|0 | 0.04 | 0.06 | 0.04 | 0.06 | 0.04 | −4.04 | 2.33 | 0.72 |
| Q13769 | THOCS | 2\|0\|2 | 4 | 1.33 | 0\|3\|0 | 0.04 | 0.05 | 0.04 | 0.05 | 0.04 | −5.8 | 1.33 | 0.75 |
| P46100-2 | ATRX | 0\|0\|2 | 2 | 0.67 | 0\|2\|0 | 0.04 | 0.12 | 0.04 | 0.12 | 0.04 | −5.38 | 1 | 0.72 |
| Q38726 | TWISTNB | 0\|0\|2 | 2 | 0.67 | 0\|0\|2 | 0.04 | 0.12 | 0.04 | 0.12 | 0.04 | −5.38 | 3 | 0.72 |
| P49821-2 | NDUFV1 | 2\|0\|0 | 2 | 0.67 | 2\|0\|0 | 0.04 | 0.12 | 0.04 | 0.12 | 0.04 | −5.38 | 1 | 0.72 |
| P42694 | HELZ | 0\|2\|2 | 4 | 1.33 | 3\|0\|0 | 0.04 | 0.05 | 0.04 | 0.05 | 0.04 | −5.8 | 1.33 | 0.75 |
| Q8IW45 | NAXD | 2\|0\|0 | 2 | 0.67 | 0\|0\|2 | 0.04 | 0.12 | 0.04 | 0.12 | 0.04 | −5.38 | 1 | 0.72 |
| Q9HCD6 | TANC2 | 2\|0\|0 | 2 | 0.67 | 2\|0\|0 | 0.04 | 0.12 | 0.04 | 0.12 | 0.04 | −5.38 | 1 | 0.72 |
| P46459 | NSF | 2\|0\|0 | 2 | 0.67 | 2\|0\|0 | 0.04 | 0.12 | 0.04 | 0.12 | 0.04 | −5.38 | 1 | 0.72 |
| Q9UJA5 | TRMT6 | 2\|0\|2 | 4 | 1.33 | 3\|0\|0 | 0.04 | 0.05 | 0.04 | 0.05 | 0.04 | −5.8 | 1.33 | 0.75 |
| Q6PKG0-3 | LARP1 | 0\|0\|2 | 2 | 0.67 | 2\|0\|0 | 0.04 | 0.12 | 0.04 | 0.12 | 0.04 | −5.38 | 1 | 0.72 |
| Q96F86 | EDC3 | 2\|2\|0 | 4 | 1.33 | 0\|0\|3 | 0.04 | 0.05 | 0.04 | 0.05 | 0.04 | −5.8 | 1.33 | 0.75 |
| Q9Y2A7 | NCKAP1 | 0\|2\|0 | 2 | 0.67 | 0\|2\|0 | 0.04 | 0.12 | 0.04 | 0.12 | 0.04 | −5.38 | 1 | 0.72 |
| Q15018 | ABRAXAS2 | 3\|3\|3 | 9 | 3 | 2\|2\|0 | 0.04 | 0.04 | 0.04 | 0.04 | 0.04 | −3.21 | 2.25 | 0.72 |
| Q15555-4 | MAPRE2 | 2\|0\|0 | 2 | 0.67 | 0\|0\|2 | 0.04 | 0.12 | 0.04 | 0.12 | 0.04 | −5.38 | 1 | 0.72 |
| P51553 | IDH3G | 3\|3\|3 | 9 | 3 | 2\|2\|0 | 0.04 | 0.04 | 0.04 | 0.04 | 0.04 | −3.21 | 2.25 | 0.72 |
| P60660 | MYL6 | 0\|2\|0 | 2 | 0.67 | 2\|0\|0 | 0.04 | 0.12 | 0.04 | 0.12 | 0.04 | −5.38 | 1 | 0.72 |
| O15084 | ANKRD28 | 0\|2\|0 | 2 | 0.67 | 2\|0\|0 | 0.04 | 0.12 | 0.04 | 0.12 | 0.04 | −5.38 | 1 | 0.72 |
| Q9NX63 | CHCHD3 | 0\|0\|2 | 2 | 0.67 | 0\|2\|0 | 0.04 | 0.12 | 0.04 | 0.12 | 0.04 | −5.38 | 3 | 0.72 |
| P62316 | SNRPD2 | 2\|0\|2 | 4 | 1.33 | 3\|0\|0 | 0.04 | 0.05 | 0.04 | 0.05 | 0.04 | −5.8 | 1.33 | 0.75 |
| Q8N0X7 | SPG20 | 0\|2\|2 | 4 | 1.33 | 0\|0\|3 | 0.04 | 0.05 | 0.04 | 0.05 | 0.04 | −5.8 | 1.33 | 0.75 |
| P12268 | IMPDH2 | 0\|0\|2 | 2 | 0.67 | 2\|0\|0 | 0.04 | 0.12 | 0.04 | 0.12 | 0.04 | −5.38 | 1 | 0.72 |
| P30260 | CDC27 | 0\|2\|0 | 2 | 0.67 | 0\|0\|2 | 0.04 | 0.12 | 0.04 | 0.12 | 0.04 | −5.38 | 1 | 0.72 |
| Q14643-4 | ITPR1 | 0\|2\|2 | 4 | 1.33 | 3\|0\|0 | 0.04 | 0.05 | 0.04 | 0.05 | 0.04 | −5.8 | 1.33 | 0.75 |
| O8NBM4 | UBAC2 | 2\|0\|0 | 2 | 0.67 | 2\|0\|0 | 0.04 | 0.12 | 0.04 | 0.12 | 0.04 | −5.38 | 1 | 0.72 |
| O60333-3 | KIF1B | 0\|0\|2 | 2 | 0.67 | 0\|0\|2 | 0.04 | 0.12 | 0.04 | 0.12 | 0.04 | −5.38 | 1 | 0.72 |
| Q86WB0 | ZC3HC1 | 0\|0\|2 | 2 | 0.67 | 0\|0\|2 | 0.04 | 0.12 | 0.04 | 0.12 | 0.04 | −5.38 | 1 | 0.72 |
| O15305 | PMM2 | 0\|2\|0 | 2 | 0.67 | 0\|0\|2 | 0.04 | 0.12 | 0.04 | 0.12 | 0.04 | −5.38 | 1 | 0.72 |
| P41236 | PPP1R2 | 2\|0\|0 | 2 | 0.67 | 0\|2\|0 | 0.04 | 0.12 | 0.04 | 0.12 | 0.04 | −5.38 | 1 | 0.72 |
| Q9BVC4-4 | MLST8 | 0\|2\|0 | 2 | 0.67 | 0\|0\|2 | 0.04 | 0.12 | 0.04 | 0.12 | 0.04 | −5.38 | 1 | 0.72 |
| P53007 | SLC25A1 | 0\|2\|0 | 2 | 0.67 | 0\|0\|2 | 0.04 | 0.12 | 0.04 | 0.12 | 0.04 | −5.38 | 1 | 0.72 |
| QSTCZ1-3 | SH3PXD2A | 0\|0\|2 | 2 | 0.67 | 0\|0\|2 | 0.04 | 0.12 | 0.04 | 0.12 | 0.04 | −5.38 | 3 | 0.72 |
| O15460 | P4HA2 | 0\|2\|0 | 2 | 0.67 | 0\|2\|0 | 0.04 | 0.12 | 0.04 | 0.12 | 0.04 | −5.38 | 1 | 0.72 |
| Q9BZE1 | MRPL37 | 2\|0\|3 | 5 | 1.67 | 4\|0\|0 | 0.03 | 0.07 | 0.03 | 0.07 | 0.03 | −7.28 | 1.25 | 0.76 |
| Q9H078-4 | CLPB | 3\|2\|3 | 8 | 2.67 | 2\|0\|2 | 0.03 | 0.04 | 0.03 | 0.04 | 0.03 | −4.67 | 2 | 0.75 |
| P61160 | ACTR2 | 2\|4\|0 | 6 | 2 | 5\|0\|0 | 0.03 | 0.07 | 0.03 | 0.07 | 0.03 | −8.76 | 1.2 | 0.76 |
| P61224-3 | RAP1B | 3\|4\|3 | 10 | 3.33 | 0\|2\|3 | 0.03 | 0.05 | 0.03 | 0.05 | 0.03 | −4.25 | 2 | 0.76 |
| QST7N3 | KANK4 | 4\|4\|2 | 10 | 3.33 | 0\|2\|3 | 0.03 | 0.05 | 0.03 | 0.05 | 0.03 | −5.66 | 2 | 0.75 |
| Q8NFH4 | NUP37 | 3\|4\|5 | 12 | 4 | 0\|3\|3 | 0.03 | 0.06 | 0.03 | 0.06 | 0.03 | −5.36 | 2 | 0.76 |
| Q86X95-2 | CIR1 | 2\|3\|3 | 8 | 2.67 | 0\|2\|2 | 0.03 | 0.04 | 0.03 | 0.04 | 0.03 | −4.67 | 2 | 0.75 |
| Q81YB8 | SUPV3L1 | 4\|3\|3 | 10 | 3.33 | 3\|0\|2 | 0.03 | 0.05 | 0.03 | 0.05 | 0.03 | −4.25 | 2 | 0.76 |
| P78346 | RPP30 | 2\|2\|4 | 8 | 2.67 | 0\|3\|2 | 0.02 | 0.05 | 0.02 | 0.05 | 0.02 | −5.66 | 1.6 | 0.76 |
| Q16775-2 | HAGH | 0\|0\|2 | 2 | 0.67 | 0\|0\|3 | 0.02 | 0.05 | 0.02 | 0.05 | 0.02 | −5.8 | 0.67 | 0.76 |
| Q9UGP4 | LIMD1 | 2\|0\|3 | 5 | 1.67 | 0\|2\|2 | 0.02 | 0.04 | 0.02 | 0.04 | 0.02 | −7.8 | 1.25 | 0.77 |
| Q13347 | EIF31 | 2\|0\|6 | 8 | 2.67 | 3\|0\|4 | 0.02 | 0.06 | 0.02 | 0.06 | 0.02 | −11.76 | 1.14 | 0.76 |
| Q9H9T3-2 | ELP3 | 0\|0\|2 | 2 | 0.67 | 0\|0\|3 | 0.02 | 0.05 | 0.02 | 0.05 | 0.02 | −5.8 | 0.67 | 0.76 |
| Q9Y320-2 | TMX2 | 0\|2\|3 | 5 | 1.67 | 0\|2\|2 | 0.02 | 0.04 | 0.02 | 0.04 | 0.02 | −7.8 | 1.25 | 0.77 |
| Q9NPI6-2 | DCP1A | 0\|2\|0 | 2 | 0.67 | 3\|0\|0 | 0.02 | 0.05 | 0.02 | 0.05 | 0.02 | −5.8 | 0.67 | 0.76 |
| Q04864-2 | REL | 3\|2\|4 | 9 | 3 | 2\|3\|0 | 0.02 | 0.05 | 0.02 | 0.05 | 0.02 | −5.66 | 1.8 | 0.76 |
| Q15813 | TBCE | 0\|2\|0 | 2 | 0.67 | 0\|3\|0 | 0.02 | 0.05 | 0.02 | 0.05 | 0.02 | −5.8 | 0.67 | 0.76 |
| O43592 | XPOT | 2\|0\|3 | 5 | 1.67 | 0\|2\|2 | 0.02 | 0.04 | 0.02 | 0.04 | 0.02 | −7.8 | 1.25 | 0.77 |
| Q13951 | CBFB | 2\|2\|3 | 7 | 2.33 | 2\|0\|2 | 0.02 | 0.04 | 0.02 | 0.04 | 0.02 | −4.67 | 1.75 | 0.76 |
| Q9BUK6-7 | MSTO1 | 2\|3\|0 | 5 | 1.67 | 2\|2\|0 | 0.02 | 0.04 | 0.02 | 0.04 | 0.02 | −7.8 | 1.25 | 0.77 |
| O60841 | EIF5B | 2\|4\|0 | 6 | 2 | 2\|3\|0 | 0.02 | 0.05 | 0.02 | 0.05 | 0.02 | −9.13 | 1.2 | 0.77 |
| Q9NSV4-7 | DIAPH3 | 2\|0\|3 | 5 | 1.67 | 0\|2\|2 | 0.02 | 0.04 | 0.02 | 0.04 | 0.02 | −7.8 | 1.25 | 0.77 |
| Q13951-2 | CBFB | 2\|2\|3 | 7 | 2.33 | 2\|0\|2 | 0.02 | 0.04 | 0.02 | 0.04 | 0.02 | −4.67 | 1.75 | 0.76 |
| Q5T457-3 | UBR4 | 4\|8\|4 | 16 | 5.33 | 5\|0\|4 | 0.02 | 0.06 | 0.02 | 0.06 | 0.02 | −7.2 | 1.78 | 0.76 |
| Q14195 | DPYSL3 | 0\|2\|0 | 2 | 0.67 | 0\|3\|0 | 0.02 | 0.05 | 0.02 | 0.05 | 0.02 | −5.8 | 0.67 | 0.76 |
| Q8NC51-4 | SERBP1 | 9\|6\|7 | 22 | 7.33 | 4\|3\|4 | 0.02 | 0.04 | 0.02 | 0.04 | 0.02 | −7.05 | 2 | 0.77 |
| Q8IZH2-2 | XRN1 | 2\|4\|2 | 8 | 2.67 | 0\|2\|3 | 0.02 | 0.05 | 0.02 | 0.05 | 0.02 | −5.66 | 1.6 | 0.76 |
| Q9Y223-4 | GNE | 0\|3\|2 | 5 | 1.67 | 2\|0\|2 | 0.02 | 0.04 | 0.02 | 0.04 | 0.02 | −7.8 | 1.25 | 0.77 |
| P12931 | SRC | 2\|0\|3 | 5 | 1.67 | 2\|2\|0 | 0.02 | 0.04 | 0.02 | 0.04 | 0.02 | −7.8 | 1.25 | 0.77 |
| O96900 | RPL36AL | 0\|2\|0 | 2 | 0.67 | 0\|3\|0 | 0.02 | 0.05 | 0.02 | 0.05 | 0.02 | −5.8 | 0.67 | 0.76 |
| Q7KZ17-15 | MARK2 | 8\|10\|7 | 25 | 8.33 | 4\|3\|5 | 0.02 | 0.05 | 0.02 | 0.05 | 0.02 | −6.82 | 2.08 | 0.76 |
| Q49A26-5 | GLYR1 | 3\|2\|0 | 5 | 1.67 | 2\|0\|2 | 0.02 | 0.04 | 0.02 | 0.04 | 0.02 | −7.8 | 1.25 | 0.77 |
| Q96A65 | EXOC4 | 4\|3\|2 | 9 | 3 | 2\|3\|0 | 0.02 | 0.05 | 0.02 | 0.05 | 0.02 | −5.66 | 1.8 | 0.76 |
| P13798 | APEH | 2\|0\|0 | 2 | 0.67 | 3\|0\|0 | 0.02 | 0.05 | 0.02 | 0.05 | 0.02 | −5.8 | 0.67 | 0.76 |
| Q96SK2-3 | TMEM209 | 2\|0\|0 | 2 | 0.67 | 0\|0\|3 | 0.02 | 0.05 | 0.02 | 0.05 | 0.02 | −5.8 | 0.67 | 0.76 |
| P10398 | ARAF | 4\|0\|0 | 4 | 1.33 | 3\|0\|2 | 0.02 | 0.05 | 0.02 | 0.05 | 0.02 | −9.13 | 0.8 | 0.77 |
| O96008 | TOMM40 | 0\|3\|7 | 10 | 3.33 | 2\|6\|0 | 0.02 | 0.06 | 0.02 | 0.06 | 0.02 | −13.01 | 1.25 | 0.76 |
| O60427 | FADS1 | 2\|3\|0 | 5 | 1.67 | 0\|2\|2 | 0.02 | 0.04 | 0.02 | 0.04 | 0.02 | −7.8 | 1.25 | 0.77 |
| Q9H357 | PTPN23 | 0\|3\|2 | 5 | 1.67 | 0\|2\|2 | 0.02 | 0.04 | 0.02 | 0.04 | 0.02 | −7.8 | 1.25 | 0.77 |
| Q14938-6 | NFIX | 3\|0\|2 | 5 | 1.67 | 2\|0\|2 | 0.02 | 0.04 | 0.02 | 0.04 | 0.02 | −7.8 | 1.25 | 0.77 |
| Q9NVV4 | MTPAP | 3\|0\|0 | 3 | 1 | 0\|2\|2 | 0.01 | 0.04 | 0.01 | 0.04 | 0.01 | −7.8 | 0.75 | 0.78 |
| Q8NSF7 | NKAP | 0\|2\|2 | 4 | 1.33 | 0\|4\|0 | 0.01 | 0.02 | 0.01 | 0.02 | 0.01 | −7.28 | 1 | 0.78 |
| Q12834 | CDC20 | 8\|3\|7 | 18 | 6 | 4\|4\|3 | 0.01 | 0.01 | 0.01 | 0.01 | 0.01 | −11.36 | 1.64 | 0.81 |
| O75436 | VPS26A | 2\|3\|3 | 8 | 2.67 | 3\|0\|2 | 0.01 | 0.01 | 0.01 | 0.01 | 0.01 | −5.66 | 1.6 | 0.79 |
| Q9BZE4 | GTPBP4 | 0\|0\|3 | 3 | 1 | 2\|0\|2 | 0.01 | 0.04 | 0.01 | 0.04 | 0.01 | −7.8 | 0.75 | 0.78 |
| Q5TDH0 | DDI2 | 7\|5\|4 | 16 | 5.33 | 5\|2\|2 | 0.01 | 0.03 | 0.01 | 0.03 | 0.01 | −7.54 | 1.78 | 0.79 |
| O60762 | DPM1 | 2\|2\|2 | 6 | 2 | 2\|2\|0 | 0.01 | 0.01 | 0.01 | 0.01 | 0.01 | −4.67 | 1.5 | 0.79 |
| Q8WU90 | ZC3H15 | 2\|0\|3 | 5 | 1.67 | 3\|2\|0 | 0.01 | 0.01 | 0.01 | 0.01 | 0.01 | −9.13 | 1 | 0.81 |
| P50579-2 | METAP2 | 2\|0\|2 | 4 | 1.33 | 2\|0\|2 | 0.01 | 0.01 | 0.01 | 0.01 | 0.01 | −7.8 | 3 | 0.8 |
| Q9BV20 | MRI1 | 3\|2\|4 | 9 | 3 | 3\|0\|3 | 0.01 | 0.02 | 0.01 | 0.02 | 0.01 | −6.82 | 1.5 | 0.8 |
| Q96H79 | ZC3HAV1L | 3\|0\|0 | 3 | 1 | 0\|2\|2 | 0.01 | 0.04 | 0.01 | 0.04 | 0.01 | −7.8 | 0.75 | 0.78 |
| Q53GA4 | PHLDA2 | 2\|2\|2 | 6 | 2 | 2\|2\|0 | 0.01 | 0.01 | 0.01 | 0.01 | 0.01 | −4.67 | 1.5 | 0.79 |
| Q9BSJ8 | ESYT1 | 0\|4\|2 | 6 | 2 | 2\|2\|2 | 0.01 | 0.02 | 0.01 | 0.02 | 0.01 | −10.44 | 1 | 0.81 |
| Q15369-2 | ELOC | 2\|0\|2 | 4 | 1.33 | 2\|2\|0 | 0.01 | 0.01 | 0.01 | 0.01 | 0.01 | −7.8 | 1 | 0.8 |
| Q9H3P7 | ACBD3 | 4\|4\|0 | 8 | 2.67 | 2\|4\|0 | 0.01 | 0.02 | 0.01 | 0.02 | 0.01 | −10.2 | 1.33 | 0.78 |
| Q8N4C8-5 | MINK1 | 4\|0\|6 | 10 | 3.33 | 2\|3\|3 | 0.01 | 0.02 | 0.01 | 0.02 | 0.01 | −13.39 | 1.25 | 0.79 |
| Q7L2H7-2 | EIF3M | 2\|2\|2 | 6 | 2 | 0\|2\|2 | 0.01 | 0.01 | 0.01 | 0.01 | 0.01 | −4.67 | 1.5 | 0.79 |
| 43307 | SSR1 | 0\|0\|3 | 3 | 1 | 0\|2\|2 | 0.01 | 0.04 | 0.01 | 0.04 | 0.01 | −7.8 | 0.75 | 0.78 |
| P29372-5 | MPG | 4\|3\|0 | 7 | 2.33 | 4\|2\|0 | 0.01 | 0.02 | 0.01 | 0.02 | 0.01 | −10.2 | 1.17 | 0.8 |
| O75340 | PDCD6 | 4\|0\|3 | 7 | 2.33 | 2\|2\|2 | 0.01 | 0.02 | 0.01 | 0.02 | 0.01 | −10.44 | 1.17 | 0.8 |
| Q8TB61-3 | SLC35B2 | 2\|0\|2 | 4 | 1.33 | 0\|2\|2 | 0.01 | 0.01 | 0.01 | 0.01 | 0.01 | −7.8 | 1 | 0.8 |
| 29YSQ9 | GTF3C3 | 0\|0\|3 | 3 | 1 | 2\|0\|2 | 0.01 | 0.04 | 0.01 | 0.04 | 0.01 | −7.8 | 0.75 | 0.78 |
| Q7L2J0 | MEPCE | 0\|0\|3 | 3 | 1 | 2\|2\|0 | 0.01 | 0.04 | 0.01 | 0.04 | 0.01 | −7.8 | 0.75 | 0.78 |
| Q5JTZ9 | AARS2 | 4\|3\|2 | 9 | 3 | 2\|2\|2 | 0.01 | 0.02 | 0.01 | 0.02 | 0.01 | −6.91 | 1.5 | 0.8 |
| Q9YSA9-2 | YTHDF2 | 5\|3\|2 | 10 | 3.33 | 3\|2\|2 | 0.01 | 0.02 | 0.01 | 0.02 | 0.01 | −8.1 | 1.43 | 0.8 |
| Q9BTE6 | AARSD1 | 7\|4\|5 | 16 | 5.33 | 2\|3\|4 | 0.01 | 0.03 | 0.01 | 0.03 | 0.01 | −7.54 | 1.78 | 0.79 |
| Q9Y653-5 | ADGRG1 | 3\|0\|0 | 3 | 1 | 0\|2\|2 | 0.01 | 0.04 | 0.01 | 0.04 | 0.01 | −7.8 | 0.75 | 0.78 |
| Q7L5Y9-3 | MAEA | 3\|3\|3 | 9 | 3 | 2\|0\|3 | 0.01 | 0.01 | 0.01 | 0.01 | 0.01 | −4.25 | 1.8 | 0.78 |
| Q9GZR7 | DDX24 | 3\|2\|7 | 12 | 4 | 3\|2\|4 | 0.01 | 0.03 | 0.01 | 0.03 | 0.01 | −10.42 | 1.33 | 0.79 |
| P67809 | YBX3 | 7\|7\|7 | 21 | 7 | 4\|2\|4 | 0.01 | 0.01 | 0.01 | 0.01 | 0.01 | −4.57 | 2.1 | 0.79 |
| P25787 | PSMA2 | 2\|0\|2 | 4 | 1.33 | 0\|2\|2 | 0.01 | 0.01 | 0.01 | 0.01 | 0.01 | −7.8 | 1 | 0.8 |
| Q96U6 | GMPPA | 2\|2\|2 | 6 | 2 | 0\|2\|2 | 0.01 | 0.01 | 0.01 | 0.01 | 0.01 | −4.67 | 1.5 | 0.79 |
| Q9H2M9 | RAB3GAP2 | 2\|0\|2 | 4 | 1.33 | 2\|0\|2 | 0.01 | 0.01 | 0.01 | 0.01 | 0.01 | −7.8 | 1 | 0.8 |
| Q9HOU3 | MAGT1 | 2\|2\|0 | 4 | 1.33 | 2\|0\|2 | 0.01 | 0.01 | 0.01 | 0.01 | 0.01 | −7.8 | 1 | 0.8 |
| Q96EY5 | MVB12A | 2\|3\|3 | 8 | 2.67 | 0\|2\|3 | 0.01 | 0.01 | 0.01 | 0.01 | 0.01 | −5.66 | 1.6 | 0.79 |
| O95831-3 | AIFM1 | 2\|2\|3 | 7 | 2.33 | 2\|0\|3 | 0.01 | 0.01 | 0.01 | 0.01 | 0.01 | −5.66 | 1.4 | 0.8 |
| Q9UJZ1-2 | STOML2 | 0\|3\|0 | 3 | 1 | 2\|0\|2 | 0.01 | 0.04 | 0.01 | 0.04 | 0.01 | −7.8 | 0.75 | 0.78 |
| Q8N556 | AFAP1 | 2\|3\|3 | 8 | 2.67 | 3\|2\|0 | 0.01 | 0.01 | 0.01 | 0.01 | 0.01 | 5.66 | 1.6 | 0.79 |
| Q12788 | TBL3 | 7\|8\|8 | 23 | 7.67 | 3\|3\|5 | 0.01 | 0.01 | 0.01 | 0.01 | 0.01 | −5.69 | 2.09 | 0.79 |
| Q13418 | ILK | 0\|3\|0 | 3 | 1 | 2\|2\|0 | 0.01 | 0.04 | 0.01 | 0.04 | 0.01 | −7.8 | 0.75 | 0.78 |
| Q9P265 | DIP28 | 2\|2\|4 | 8 | 2.67 | 2\|4\|0 | 0.01 | 0.02 | 0.01 | 0.02 | 0.01 | −6.67 | 1.33 | 0.8 |
| P04083 | ANXA1 | 2\|2\|4 | 8 | 2.67 | 4\|2\|0 | 0.01 | 0.02 | 0.01 | 0.02 | 0.01 | −6.67 | 1.33 | 0.8 |
| Q9H089 | LSG1 | 2\|0\|2 | 4 | 1.33 | 0\|2\|2 | 0.01 | 0.01 | 0.01 | 0.01 | 0.01 | −7.8 | 3 | 0.8 |
| P11766 | ADHS | 0\|5\|3 | 8 | 2.67 | 2\|3\|2 | 0.01 | 0.02 | 0.01 | 0.02 | 0.01 | −11.93 | 1.14 | 0.8 |
| Q92905 | COPSS | 4\|2\|7 | 13 | 4.33 | 4\|2\|3 | 0.01 | 0.03 | 0.01 | 0.03 | 0.01 | −10.42 | 1.44 | 0.79 |
| Q9ULC3 | RAB23 | 2\|0\|2 | 4 | 1.33 | 0\|2\|2 | 0.01 | 0.01 | 0.01 | 0.01 | 0.01 | −7.8 | 1 | 0.8 |
| O43237-2 | DYNC1LI2 | 3\|3\|4 | 10 | 3.33 | 0\|3\|3 | 0.01 | 0.02 | 0.01 | 0.02 | 0.01 | −5.36 | 1.67 | 0.79 |
| Q8N684-2 | ICPSF7 | 0\|0\|3 | 3 | 1 | 0\|2\|2 | 0.01 | 0.04 | 0.01 | 0.04 | 0.01 | −7.8 | 0.75 | 0.78 |
| Q15019 | 2-Sep | 2\|2\|0 | 4 | 1.33 | 2\|0\|2 | 0.01 | 0.01 | 0.01 | 0.01 | 0.01 | −7.8 | 1 | 0.8 |
| O95817 | BAG3 | 4\|5\|7 | 16 | 5.33 | 3\|4\|2 | 0.01 | 0.03 | 0.01 | 0.03 | 0.01 | −7.54 | 1.78 | 0.79 |
| P06753-3 | TPM3 | 2\|0\|2 | 4 | 1.33 | 2\|0\|2 | 0.01 | 0.01 | 0.01 | 0.01 | 0.01 | −7.8 | 1 | 0.8 |
| O14694 | USP10 | 2\|3\|2 | 7 | 2.33 | 3\|0\|2 | 0.01 | 0.01 | 0.01 | 0.01 | 0.01 | −5.66 | 1.4 | 0.8 |
| O14818 | PSMA7 | 2\|2\|0 | 4 | 1.33 | 0\|2\|2 | 0.01 | 0.01 | 0.01 | 0.01 | 0.01 | −7.8 | 1 | 0.8 |
| Q6DKJ4-3 | NXN | 2\|0\|2 | 4 | 1.33 | 0\|2\|2 | 0.01 | 0.01 | 0.01 | 0.01 | 0.01 | −7.8 | 3 | 0.8 |
| Q96Q11-2 | TRNT1 | 2\|2\|0 | 4 | 1.33 | 2\|0\|2 | 0.01 | 0.01 | 0.01 | 0.01 | 0.01 | −7.8 | 3 | 0.8 |
| Q10567-4 | AP181 | 2\|0\|2 | 4 | 1.33 | 2\|0\|2 | 0.01 | 0.01 | 0.01 | 0.01 | 0.01 | −7.8 | 1 | 0.8 |
| Q8TEU7-5 | RAPGEF6 | 3\|4\|2 | 9 | 3 | 2\|2\|2 | 0.01 | 0.02 | 0.01 | 0.02 | 0.01 | −6.91 | 1.5 | 0.8 |
| O5VV42 | CDKAL1 | 0\|2\|4 | 6 | 2 | 3\|3\|0 | 0.01 | 0.02 | 0.01 | 0.02 | 0.01 | −10.34 | 1 | 0.81 |
| Q9H910 | JPT2 | 3\|3\|4 | 10 | 3.33 | 0\|3\|3 | 0.01 | 0.02 | 0.01 | 0.02 | 0.01 | −5.36 | 1.67 | 0.79 |
| Q8TDN6 | BRIX1 | 0\|0\|3 | 3 | 1 | 2\|2\|0 | 0.01 | 0.04 | 0.01 | 0.04 | 0.01 | −7.8 | 0.75 | 0.78 |
| O14545 | TRAFD1 | 2\|3\|3 | 8 | 2.67 | 3\|0\|2 | 0.01 | 0.01 | 0.01 | 0.01 | 0.01 | −5.66 | 1.6 | 0.79 |
| Q9Y666 | SLC12A7 | 3\|3\|4 | 10 | 3.33 | 2\|4\|0 | 0.01 | 0.02 | 0.01 | 0.02 | 0.01 | −5.2 | 1.67 | 0.79 |
| O43159 | RRP8 | 0\|0\|3 | 3 | 1 | 2\|0\|2 | 0.01 | 0.04 | 0.01 | 0.04 | 0.01 | −7.8 | 0.75 | 0.78 |
| P46977 | STT3A | 5\|5\|2 | 12 | 4 | 0\|4\|3 | 0.01 | 0.02 | 0.01 | 0.02 | 0.01 | −7.93 | 1.71 | 0.78 |
| Q8NBF2-2 | NHLRC2 | 2\|2\|0 | 4 | 1.33 | 2\|0\|2 | 0.01 | 0.01 | 0.01 | 0.01 | 0.01 | −7.8 | 1 | 0.8 |
| P12694 | BCKDHA | 0\|6\|5 | 11 | 3.67 | 3\|3\|2 | 0.01 | 0.02 | 0.01 | 0.02 | 0.01 | −13.39 | 1.38 | 0.79 |
| Q86VF2-5 | IGFN1 | 0\|2\|0 | 2 | 0.67 | 4\|0\|0 | 0.01 | 0.02 | 0.01 | 0.02 | 0.01 | −7.28 | 0.5 | 0.8 |
| Q9NWT1 | PAK1IP1 | 3\|2\|0 | 5 | 1.67 | 3\|0\|2 | 0.01 | 0.01 | 0.01 | 0.01 | 0.01 | −9.13 | 1 | 0.81 |
| Q9Y312 | AAR2 | 4\|0\|0 | 4 | 1.33 | 4\|2\|0 | 0.01 | 0.02 | 0.01 | 0.02 | 0.01 | −10.2 | 0.67 | 0.8 |
| Q8TCU4-3 | ALMS1 | 5\|3\|5 | 13 | 4.33 | 2\|3\|2 | 0.01 | 0.02 | 0.01 | 0.02 | 0.01 | −6.63 | 1.86 | 0.78 |
| Q6ZSR9 | Q6ZSR9 | 4\|3\|2 | 9 | 3 | 2\|2\|2 | 0.01 | 0.02 | 0.01 | 0.02 | 0.01 | −6.91 | 1.5 | 0.8 |
| P49005 | POLD2 | 8\|7\|6 | 21 | 7 | 5\|4\|2 | 0.01 | 0.01 | 0.01 | 0.01 | 0.01 | −7.05 | 1.91 | 0.81 |
| Q9Y3D8 | AK6 | 2\|0\|3 | 5 | 1.67 | 3\|2\|0 | 0.01 | 0.01 | 0.01 | 0.01 | 0.01 | −9.13 | 1 | 0.81 |
| Q15738 | NSDHL | 8\|5\|3 | 16 | 5.33 | 2\|3\|5 | 0.01 | 0.04 | 0.01 | 0.04 | 0.01 | −10.17 | 1.6 | 0.79 |
| P17098 | ZNF8 | 2\|2\|0 | 4 | 1.33 | 2\|0\|2 | 0.01 | 0.01 | 0.01 | 0.01 | 0.01 | −7.8 | 1 | 0.8 |
| Q9Y6Y8 | SEC23IP | 4\|4\|4 | 12 | 4 | 3\|2\|2 | 0.01 | 0.01 | 0.01 | 0.01 | 0.01 | −5.22 | 1.71 | 0.81 |
| Q8N884 | MB21D1 | 2\|2\|2 | 6 | 2 | 0\|2\|2 | 0.01 | 0.01 | 0.01 | 0.01 | 0.01 | −4.67 | 1.5 | 0.79 |
| P61923 | COPZ1 | 2\|0\|2 | 4 | 1.33 | 0\|2\|2 | 0.01 | 0.01 | 0.01 | 0.01 | 0.01 | −7.8 | 1 | 0.8 |
| Q96G03 | PGM2 | 0\|3\|2 | 5 | 1.67 | 3\|0\|2 | 0.01 | 0.01 | 0.01 | 0.01 | 0.01 | −9.13 | 1 | 0.81 |
| O92530 | PSMF1 | 0\|2\|0 | 2 | 0.67 | 4\|0\|0 | 0.01 | 0.02 | 0.01 | 0.02 | 0.01 | −7.28 | 0.5 | 0.8 |
| Q15056-2 | EIF4H | 3\|2\|3 | 8 | 2.67 | 0\|3\|2 | 0.01 | 0.01 | 0.01 | 0.01 | 0.01 | −5.66 | 1.6 | 0.79 |
| Q14974 | KPNB1 | 2\|2\|0 | 4 | 1.33 | 2\|2\|0 | 0.01 | 0.01 | 0.01 | 0.01 | 0.01 | −7.8 | 1 | 0.8 |
| P40227 | CCT6A | 36\|34\|34 | 104 | 34.67 | 33\|35\|44 | 0 | 0 | 0 | 0 | 0 | −85.12 | 0.93 | 0.92 |
| P24941-2 | CDK2 | 5\|5\|5 | 15 | 5 | 5\|4\|6 | 0 | 0 | 0 | 0 | 0 | −13.14 | 1 | 0.92 |
| P16435 | POR | 5\|6\|4 | 15 | 5 | 9\|3\|8 | 0 | 0 | 0 | 0 | 0 | −20.73 | 0.75 | 0.92 |
| P84077 | ARF1 | 14\|13\|17 | 44 | 14.67 | 18\|14\|16 | 0 | 0 | 0 | 0 | 0 | −40.47 | 0.92 | 0.92 |
| P51610-4 | HCFC1 | 0\|2\|0 | 2 | 0.67 | 13\|5\|5 | 0 | 0 | 0 | 0 | 0 | −34.96 | 0.09 | 0.92 |
| Q00535 | CDKS | 4\|4\|4 | 12 | 4 | 4\|4\|3 | 0 | 0 | 0 | 0 | 0 | −9.88 | 1.09 | 0.88 |
| Q00534 | CDK6 | 10\|9\|8 | 27 | 9 | 8\|7\|7 | 0 | 0 | 0 | 0 | 0 | −17.01 | 1.23 | 0.9 |
| O00763-3 | ACACB | 07\|107\|11 | 327 | 109 | 16\|18\|10 | 0 | 0 | 0 | 0 | 0 | −248.32 | 0.96 | 0.92 |
| Q9UMZ2-8 | SYNRG | 9\|6\|6 | 21 | 7 | 11\|9\|7 | 0 | 0 | 0 | 0 | 0 | −26.11 | 0.78 | 0.92 |
| POC0S5 | H2AFZ | 2\|2\|2 | 6 | 2 | 2\|2\|2 | 0 | 0 | 0 | 0 | 0 | −6.91 | 3 | 0.92 |
| Q9UBM7 | DHCR7 | 2\|2\|0 | 0 | 1.33 | 0\|3\|2 | 0 | 0 | 0 | 0 | 0 | −9.13 | 0.8 | 0.83 |
| P02545 | LMNA | 12\|12\|14 | 38 | 12.67 | 17\|16\|20 | 0 | 0 | 0 | 0 | 0 | −48.41 | 0.72 | 0.92 |
| P40429 | RPL13A | 9\|9\|6 | 24 | 8 | 9\|10\|8 | 0 | 0 | 0 | 0 | 0 | −26.11 | 0.89 | 0.92 |
| Q7Z3T8 | ZFYVE16 | 5\|5\|3 | 13 | 4.33 | 11\|3\|9 | 0 | 0 | 0 | 0 | 0 | −26.5 | 0.57 | 0.92 |
| Q13838 | DDX39B | 9\|10\|11 | 30 | 10 | 11\|10\|12 | 0 | 0 | 0 | 0 | 0 | −28.56 | 0.91 | 0.92 |
| Q96T37-4 | RBM15 | 5\|5\|2 | 12 | 4 | 5\|2\|3 | 0 | 0 | 0 | 0 | 0 | −11.71 | 1.2 | 0.86 |
| P02769 | P02769 | 7\|9\|12 | 28 | 9.33 | 39\|5\|6 | 0 | 0 | 0 | 0 | 0 | −52.96 | 0.56 | 0.92 |
| P02768 | ALB | 2\|2\|2 | 6 | 2 | 4\|2\|2 | 0 | 0 | 0 | 0 | 0 | −9.29 | 0.75 | 0.92 |
| P04259 | KRT6B | 15\|14\|22 | 51 | 17 | 14\|15\|17 | 0 | 0 | 0 | 0 | 0 | −36.52 | 1.11 | 0.91 |
| P05387 | RPLP2 | 0\|4\|2 | 6 | 2 | 3\|3\|3 | 0 | 0 | 0 | 0 | 0 | −14.75 | 0.67 | 0.87 |
| P05388 | RPLP0 | 6\|0\|5 | 11 | 3.67 | 5\|5\|3 | 0 | 0 | 0 | 0 | 0 | −20.61 | 0.85 | 0.88 |
| P62979 | RPS27A | 32\|32\|29 | 93 | 31 | 25\|22\|21 | 0 | 0 | 0 | 0 | 0 | −40.71 | 1.37 | 0.92 |
| Q9BQ52-4 | ELAC2 | 5\|3\|2 | 10 | 3.33 | 2\|3\|3 | 0 | 0.01 | 0 | 0.01 | 0 | −9.29 | 1.25 | 0.83 |
| O76021 | RSL1D1 | 5\|0\|8 | 13 | 4.33 | 7\|5\|5 | 0 | 0 | 0 | 0 | 0 | −26.48 | 0.76 | 0.89 |
| Q15365 | PCBP1 | 24\|25\|25 | 74 | 24.67 | 24\|24\|24 | 0 | 0 | 0 | 0 | 0 | −52.54 | 1.03 | 0.92 |
| P29401 | TKT | 5\|3\|4 | 12 | 4 | 3\|4\|3 | 0 | 0 | 0 | 0 | 0 | −10.17 | 1.2 | 0.86 |
| Q13907 | IDI1 | 4\|4\|3 | 11 | 3.67 | 5\|6\|4 | 0 | 0 | 0 | 0 | 0 | −16.23 | 0.73 | 0.92 |
| P84085 | ARFS | 9\|7\|10 | 26 | 8.67 | 10\|6\|10 | 0 | 0 | 0 | 0 | 0 | −23.23 | 1 | 0.91 |
| Q9NY93-2 | DDX56 | 2\|2\|0 | 4 | 1.33 | 2\|0\|3 | 0 | 0 | 0 | 0 | 0 | −9.13 | 0.8 | 0.83 |
| P39023 | RPL3 | 17\|10\|21 | 48 | 16 | 23\|30\|25 | 0 | 0 | 0 | 0 | 0 | −85.6 | 0.62 | 0.92 |
| P35241 | RDX | 2\|2\|0 | 4 | 1.33 | 3\|3\|2 | 0 | 0 | 0 | 0 | 0 | −13.39 | 0.5 | 0.92 |
| O75439 | PMPCB | 11\|6\|12 | 29 | 9.67 | 9\|9\|11 | 0 | 0 | 0 | 0 | 0 | −28.68 | 1 | 0.91 |
| O95251-2 | KAT7 | 0\|3\|0 | 3 | 1 | 2\|3\|2 | 0 | 0 | 0 | 0 | 0 | −11.93 | 0.43 | 0.86 |
| Q96AC1 | FERMT2 | 13\|11\|15 | 39 | 13 | 9\|10\|14 | 0 | 0 | 0 | 0 | 0 | −25.58 | 1.18 | 0.91 |
| P62805 | HIST1H4L; | 2\|0\|0 | 2 | 0.67 | 314\|3 | 0 | 0 | 0 | 0 | 0 | −16.24 | 0.2 | 0.92 |
| | HIST1H4 | | | | | | | | | | | | |
| P10316 | HLA-A | 7\|6\|6 | 19 | 6.33 | 5\|3\|3 | 0 | 0 | 0 | 0 | 0 | −7.05 | 1.73 | 0.84 |
| P13646-3 | KRT13 | 9\|7\|7 | 23 | 7.67 | 9\|8\|6 | 0 | 0 | 0 | 0 | 0 | −19.64 | 1 | 0.91 |
| Q9H1A4 | ANAPC1 | 2\|3\|5 | 10 | 3.33 | 6\|6\|5 | 0 | 0 | 0 | 0 | 0 | −20.89 | 0.59 | 0.92 |
| Q72627-3 | IHUWE1 | 16\|10\|8 | 34 | 11.33 | 4\|7\|11 | 0 | 0 | 0 | 0 | 0 | −16.85 | 1.55 | 0.86 |
| Q09160 | HLA-A | 2\|2\|2 | 6 | 2 | 2\|3\|0 | 0 | 0 | 0 | 0 | 0 | −5.66 | 1.2 | 0.82 |
| Q99081 | TCF12 | 0\|0\|2 | 2 | 0.67 | 7\|3\|7 | 0 | 0 | 0 | 0 | 0 | −26.48 | 0.12 | 0.92 |
| Q9UKM9-2 | RALY | 2\|0\|0 | 2 | 0.67 | 2\|2\|2 | 0 | 0 | 0 | 0 | 0 | −10.44 | 0.33 | 0.92 |
| P60981-2 | DSTN | 5\|0\|3 | 8 | 2.67 | 3\|4\|6 | 0 | 0 | 0 | 0 | 0 | −20.61 | 0.62 | 0.89 |
| Q14562 | DHX8 | 2\|0\|0 | 2 | 0.67 | 0\|6\|4 | 0 | 0 | 0 | 0 | 0 | −15.87 | 0.2 | 0.92 |
| O60763 | USO1 | 4\|4\|3 | 11 | 3.67 | 5\|3\|3 | 0 | 0 | 0 | 0 | 0 | −11.36 | 3 | 0.88 |
| P18077 | RPL35A | 4\|4\|3 | 11 | 3.67 | 615\|5 | 0 | 0 | 0 | 0 | 0 | −17.53 | 0.69 | 0.92 |
| Q15181 | PPA1 | 7\|6\|11 | 24 | 8 | 7\|7\|9 | 0 | 0 | 0 | 0 | 0 | −21.17 | 1.04 | 0.9 |
| Q04637-8 | EIF4G1 | 17\|15\|17 | 49 | 16.33 | 14\|16\|13 | 0 | 0 | 0 | 0 | 0 | −31.53 | 1.14 | 0.92 |
| P17844 | DDX5 | 69\|79\|79 | 227 | 75.67 | 72\|66\|76 | 0 | 0 | 0 | 0 | 0 | −154.11 | 1.06 | 0.92 |
| Q7Z5K2 | WAPL | 0\|0\|3 | 3 | 1 | 3\|4\|2 | 0 | 0 | 0 | 0 | 0 | −14.75 | 0.33 | 0.92 |
| O94906 | PRPF6 | 0\|0\|2 | 2 | 0.67 | 5\|5\|4 | 0 | 0 | 0 | 0 | 0 | −22.08 | 0.14 | 0.92 |
| Q2TAY7 | SMU1 | 5\|4\|6 | 15 | 5 | 7\|3\|6 | | 0 | 0 | 0 | 0 | −15.82 | 0.94 | 0.9 |
| Q15054-3 | POLD3 | 3\|2\|2 | 7 | 2.33 | 2\|2\|3 | 0 | 0 | 0 | 0 | 0 | −8.1 | 1 | 0.86 |
| P04908 | HIST1H2AE; | 4\|3\|5 | 12 | 4 | 8\|6\|8 | 0 | 0 | 0 | | 0 | −25.42 | 0.55 | 0.92 |
| | HIST1H | | | | | | | | | | | | |
| Q8N3C0 | ASCC3 | 14\|14\|15 | 43 | 14.33 | 8\|11\|10 | 0 | 0 | 0 | 0 | | −16.64 | 1.48 | 0.9 |
| Q9NRF8 | CTPS2 | 2\|3\|0 | 5 | 1.67 | 2\|3\|2 | 0 | 0 | 0 | 0 | 0 | −11.93 | 0.71 | 0.86 |
| Q9BUF5 | TUBB6 | 35\|34\|37 | 106 | 35.33 | 41\|33\|34 | 0 | 0 | 0 | 0 | 0 | −80.35 | 0.98 | 0.92 |
| Q96CW5-2 | TUBGCP3 | 0\|0\|3 | 3 | 1 | 0\|2\|4 | 0 | 0.01 | 0 | 0.01 | 0 | −10.2 | 0.5 | 0.83 |
| P48960-2 | CD97 | 3\|4\|5 | 12 | 4 | 6\|2\|5 | 0 | 0 | 0 | 0 | 0 | −13.76 | 0.92 | 0.89 |
| O562R1 | ACTBL2 | 34\|32\|38 | 104 | 34.67 | 47\|36\|37 | 0 | 0 | 0 | 0 | 0 | −98.01 | 0.87 | 0.92 |
| Q02878 | RPL6 | 22\|20\|20 | 62 | 20.67 | 26\|21\|23 | 0 | 0 | 0 | 0 | 0 | −56.15 | 0.89 | 0.92 |
| P16152 | CBR1 | 3\|8\|4 | 15 | 5 | 8\|6\|5 | 0 | 0 | 0 | 0 | 0 | −21.43 | 0.79 | 0.9 |
| Q9UNX3 | RPL26L1 | 11\|13\|13 | 37 | 12.33 | 15\|11\|13 | 0 | 0 | 0 | 0 | 0 | −32.69 | 0.95 | 0.92 |
| Q9UNX4 | WDR3 | 3\|2\|2 | 7 | 2.33 | 3\|3\|0 | 0 | 0 | 0 | 0 | 0 | −6.82 | 1.17 | 0.84 |
| Q9Y5S2 | CDC42BPB | 0\|0\|2 | 2 | 0.67 | 6\|3\|3 | 0 | 0 | 0 | 0 | 0 | −19.11 | 0.17 | 0.92 |
| Q9UGP8 | SEC63 | 3\|3\|3 | 9 | 3 | 3\|2\|2 | 0 | 0 | 0 | 0 | 0 | −6.63 | 1.29 | 0.84 |
| O43776 | NARS | 12\|15\|13 | 40 | 13.33 | 12\|16\|11 | 0 | 0 | 0 | 0 | 0 | −31.17 | 1.03 | 0.91 |
| P26640 | VARS | 24\|19\|23 | 66 | 22 | 16\|19\|20 | 0 | 0 | 0 | 0 | 0 | −39.82 | 1.2 | 0.92 |
| P26641 | EEF1G | 15\|12\|17 | 44 | 14.67 | 16\|16\|16 | 0 | 0 | 0 | 0 | 0 | −42.12 | 0.92 | 0.92 |
| Q16527 | CSRP2 | 10\|10\|14 | 34 | 11.33 | 10\|9\|9 | 0 | 0 | 0 | 0 | 0 | −21.15 | 1.21 | 0.91 |
| Q71UM5 | RPS27L | 8\|8\|11 | 27 | 9 | 9\|7\|5 | 0 | 0 | 0 | 0 | 0 | −15.83 | 1.29 | 0.89 |
| P60709 | ACTB | 100\|94\|10 | 296 | 98.67 | 109\|107\|97 | 0 | 0 | 0 | 0 | 0 | −234.56 | 0.95 | 0.92 |
| Q6Y7W6-4 | GIGYF2 | 7\|9\|6 | 22 | 7.33 | 5\|11\|9 | 0 | 0 | 0 | 0 | 0 | −23.67 | 0.88 | 0.91 |
| O00203-3 | AP381 | 2\|2\|0 | 4 | 1.33 | 3\|3\|3 | 0 | 0 | 0 | 0 | 0 | −14.75 | 0.44 | 0.92 |
| Q9UHB6-5 | LIMA1 | 8\|7\|8 | 23 | 7.67 | 5\|5\|6 | 0 | 0 | 0 | 0 | 0 | −11.43 | 1.44 | 0.88 |
| Q9UHB6-2 | LIMA1 | 12\|7\|8 | 27 | 9 | 6\|5\|7 | 0 | 0 | 0 | 0 | 0 | −13.76 | 1.5 | 0.86 |
| Q9NUU7 | DDX19A | 14\|9\|10 | 33 | 11 | 8\|3\|8 | 0 | 0 | 0 | 0 | 0 | −12.07 | 1.74 | 0.84 |
| O75400-2 | PRPF40A | 2\|2\|2 | 6 | 2 | 0\|4\|2 | 0 | 0 | 0 | 0 | 0 | −6.67 | 1 | 0.92 |
| P49411 | TUFM | 27\|26\|30 | 83 | 27.67 | 33\|26\|27 | 0 | 0 | 0 | 0 | 0 | −66.16 | 0.97 | 0.92 |
| P48147 | PREP | 4\|5\|3 | 12 | 4 | 7\|3\|4 | 0 | 0 | 0 | 0 | 0 | −15.01 | 0.86 | 0.9 |
| P23588 | EIF4B | 10\|12\|8 | 30 | 10 | 10\|8\|7 | 0 | 0 | 0 | 0 | 0 | −20.53 | 1.2 | 0.9 |
| P62495 | ETF1 | 4\|4\|6 | 14 | 4.67 | 3\|3\|3 | 0 | 0.01 | 0 | 0.01 | 0 | −7.54 | 1.56 | 0.82 |
| Q14966 | ZNF638 | 0\|2\|5 | 7 | 2.33 | 9\|7\|4 | 0 | 0 | 0 | 0 | 0 | −30.9 | 0.35 | 0.92 |
| P50395 | GDI2 | 13\|14\|17 | 44 | 14.67 | 16\|19\|19 | 0 | 0 | 0 | 0 | 0 | −47.91 | 0.81 | 0.92 |
| P49841-2 | GSK3B | 7\|5\|8 | 20 | 6.67 | 4\|6\|3 | 0 | 0 | 0 | 0 | 0 | −10.78 | 1.54 | 0.85 |
| Q13643 | FHL3 | 0\|2\|2 | 4 | 1.33 | 3\|2\|2 | 0 | 0 | 0 | 0 | 0 | −11.93 | 0.57 | 0.92 |
| P25205 | MCM3 | 0\|2\|2 | 4 | 1.33 | 3\|2\|2 | 0 | 0 | 0 | 0 | 0 | −11.93 | 0.57 | 0.92 |
| Q16186 | ADRM1 | 6\|6\|5 | 17 | 5.67 | 3\|2\|6 | 0 | 0 | 0 | 0 | 0 | −8.47 | 1.55 | 0.85 |
| Q12873-2 | CHD3 | 2\|0\|2 | 4 | 1.33 | 3\|4\|5 | 0 | 0 | 0 | 0 | 0 | −19.11 | 0.33 | 0.92 |
| A0MZ66 | SHTN1 | 16\|17\|17 | 50 | 16.67 | 19\|19\|21 | 0 | 0 | 0 | 0 | 0 | −49.12 | 0.85 | 0.92 |
| Q96HS1 | PGAM5 | 3\|2\|3 | 8 | 2.67 | 6\|4\|3 | 0 | 0 | 0 | 0 | 0 | −15.55 | 0.62 | 0.92 |
| Q72478 | DHX29 | 4\|5\|2 | 11 | 3.67 | 3\|3\|2 | 0 | 0.01 | 0 | 0.01 | 0 | −9.29 | 1.38 | 0.83 |
| Q99613-2 | EIF3C | 18\|18\|17 | 53 | 17.67 | 17\|17\|13 | 0 | 0 | 0 | 0 | 0 | −33.33 | 1.13 | 0.92 |
| 21399 | ACO1 | 4\|7\|4 | 15 | 5 | 3\|6\|4 | 0 | 0 | 0 | 0 | 0 | −12.24 | 1.15 | 0.87 |
| Q12797-10 | ASPH | 4\|4\|4 | 12 | 4 | 3\|3\|2 | 0 | 0 | 0 | 0 | 0 | −6.37 | 1.5 | 0.84 |
| O15067 | PFAS | 24\|17\|24 | 65 | 21.67 | 21\|21\|25 | 0 | 0 | 0 | 0 | 0 | −57.34 | 0.97 | 0.92 |
| P10909-4 | ICLU | 89\|9 | 26 | 8.67 | 10\|8\|7 | 0 | 0 | 0 | 0 | 0 | −20.53 | 1.04 | 0.91 |
| Q9NZM5 | NOP53 | 2\|0\|0 | 2 | 0.67 | 0\|2\|2 | 0 | 0.01 | 0 | 0.01 | 0 | −7.8 | 0.5 | 0.82 |
| P31942-2 | HNRNPH3 | 3\|3\|3 | 9 | 3 | 4\|5\|4 | 0 | 0 | 0 | 0 | 0 | −13.76 | 0.69 | 0.92 |
| Q86YP4-2 | GATAD2A | 5\|5\|4 | 14 | 4.67 | 7\|5\|5 | 0 | 0 | 0 | 0 | 0 | −17.06 | 0.82 | 0.92 |
| Q9NW64 | RBM22 | 5\|4\|8 | 17 | 5.67 | 4\|7\|8 | 0 | 0 | 0 | 0 | 0 | −19.55 | 0.89 | 0.9 |
| Q9BXP5-5 | SRRT | 4\|3\|2 | 9 | 3 | 4\|4\|4 | 0 | 0 | 0 | 0 | 0 | −14.21 | 0.75 | 0.92 |
| P06748-2 | NPM1 | 4\|6\|4 | 14 | 4.67 | 9\|8\|9 | 0 | 0 | 0 | 0 | 0 | −28.65 | 0.54 | 0.92 |
| Q9Y295 | DRG1 | 7\|9\|8 | 24 | 8 | 10\|4\|4 | 0 | 0 | 0 | 0 | 0 | −13.51 | 1.33 | 0.89 |
| Q6UXN9 | WDR82 | 5\|4\|4 | 13 | 4.33 | 4\|6\|4 | 0 | 0 | 0 | 0 | 0 | −13.43 | 0.93 | 0.9 |
| P17066 | HSPA6 | 14\|13\|12 | 39 | 13 | 13\|9\|12 | 0 | 0 | 0 | 0 | 0 | −25.3 | 1.15 | 0.91 |
| P49959 | MRE11 | 4\|4\|6 | 14 | 4.67 | 6\|5\|4 | 0 | 0 | 0 | 0 | 0 | −14.57 | 0.93 | 0.89 |
| P49790 | NUP153 | 27\|26\|26 | 79 | 26.33 | 27\|29\|26 | 0 | 0 | 0 | 0 | 0 | −61.39 | 0.96 | 0.92 |
| Q01813 | PFKP | 14\|15\|14 | 43 | 14.33 | 23\|22\|21 | 0 | 0 | 0 | 0 | 0 | −61.35 | 0.65 | 0.92 |
| P49792 | RANBP2 | 48\|42\|50 | 140 | 46.67 | 64\|55\|55 | 0 | 0 | 0 | 0 | 0 | −148.4 | 0.8 | 0.92 |
| P56545 | CTBP2 | 4\|4\|3 | 11 | 3.67 | 4\|2\|5 | 0 | 0 | 0 | 0 | 0 | −11.36 | 1 | 0.88 |
| O60293-2 | ZFC3H1 | 2\|2\|2 | 6 | 2 | 0\|3\|4 | 0 | 0 | 0 | 0 | 0 | −7.93 | 0.86 | 0.92 |
| O76031 | CLPX | 6\|4\|5 | 15 | 5 | 7\|5\|4 | 0 | 0 | 0 | 0 | 0 | −15.82 | 0.94 | 0.9 |
| Q8WWK9- | CKAP2 | 0\|2\|2 | 4 | 1.33 | 2\|2\|2 | 0 | 0 | 0 | 0 | 0 | −10.44 | 0.67 | 0.92 |
| Q9NRZ9-3 | HELLS | 4\|4\|9 | 17 | 5.67 | 51\|50\|52 | 0 | 0 | 0 | 0 | 0 | −211.19 | 0.11 | 0.92 |
| Q86W42-3 | THOC6 | 3\|5\|4 | 12 | 4 | 4\|4\|4 | 0 | 0 | 0 | 0 | 0 | −12.5 | 1 | 0.88 |
| O00154-6 | ACOT7 | 5\|5\|5 | 15 | 5 | 8\|5\|4 | 0 | 0 | 0 | 0 | 0 | −15.5 | 0.88 | 0.92 |
| P62873-2 | GNB1 | 3\|5\|3 | 11 | 3.67 | 2\|3\|5 | 0 | 0 | 0 | 0 | 0 | −10.17 | 1.1 | 0.87 |
| P31483 | TIA1 | 3\|5\|5 | 13 | 4.33 | 4\|4\|4 | 0 | 0 | 0 | 0 | 0 | −12.5 | 1.08 | 0.88 |
| P26368-2 | U2AF2 | 10\|10\|11 | 31 | 10.33 | 20\|9\|7 | 0 | 0 | 0 | 0 | 0 | −30.03 | 0.86 | 0.92 |
| O00505 | KPNA3 | 2\|2\|0 | 4 | 1.33 | 3\|3\|2 | 0 | 0 | 0 | 0 | 0 | −13.39 | 0.5 | 0.92 |
| Q9Y4P3 | TBL2 | 4\|6\|3 | 13 | 4.33 | 4\|6\|4 | 0 | 0 | 0 | 0 | 0 | −15.01 | 0.93 | 0.89 |
| P08708 | RPS17 | 7\|5\|7 | 19 | 6.33 | 3\|8\|11 | 0 | 0 | 0 | 0 | 0 | −21.3 | 0.86 | 0.92 |
| Q01581 | HMGCS1 | 20\|19\|20 | 59 | 19.67 | 17\|19\|22 | 0 | 0 | 0 | 0 | 0 | −43.35 | 1.02 | 0.92 |
| P29692 | EEFID | 12\|11\|15 | 38 | 12.67 | 7\|8\|10 | 0 | 0 | 0 | 0 | 0 | −16.22 | 1.52 | 0.89 |
| P78344 | EIF4G2 | 34\|37\|35 | 106 | 35.33 | 21\|11\|14 | 0 | 0 | 0 | 0 | 0 | −10.86 | 2.3 | 0.87 |
| Q9H2U1-3 | DHX36 | 5\|6\|5 | 16 | 5.33 | 4\|3\|3 | 0 | 0 | 0 | 0 | 0 | −7.29 | 1.6 | 0.84 |
| Q9BXB5 | OSBPL10 | 3\|2\|2 | 7 | 2.33 | 2\|3\|2 | 0 | 0 | 0 | 0 | 0 | −8.1 | 1 | 0.86 |
| Q14566 | MCM6 | 13\|13\|13 | 39 | 13 | 8\|13\|16 | 0 | 0 | 0 | 0 | 0 | −27.34 | 1.05 | 0.91 |
| O00139-2 | KIF2A | 5\|6\|4 | 15 | 5 | 3\|5\|5 | 0 | 0 | 0 | 0 | 0 | −12.24 | 1.15 | 0.88 |
| Q15637-4 | SF1 | 2\|0\|2 | 4 | 1.33 | 2\|2\|3 | 0 | 0 | 0 | 0 | 0 | −11.93 | 0.57 | 0.92 |
| P62703 | RPS4X | 38\|36\|42 | 116 | 38.67 | 39\|41\|36 | 0 | 0 | 0 | 0 | 0 | −86.88 | 1 | 0.92 |
| P27694 | RPA1 | 13\|14\|14 | 41 | 13.67 | 12\|10\|19 | 0 | 0 | 0 | 0 | 0 | −31.95 | 3 | 0.92 |
| P27695 | APEX1 | 2\|2\|0 | 4 | 1.33 | 3\|0\|3 | 0 | 0 | 0 | 0 | 0 | −10.34 | 0.67 | 0.92 |
| P17858 | PFKL | 3\|4\|3 | 10 | 3.33 | 6\|7\|8 | 0 | 0 | 0 | 0 | 0 | −24.06 | 0.48 | 0.92 |
| Q9BTC0 | DIDO1 | 3\|2\|3 | 8 | 2.67 | 6\|4\|4 | 0 | 0 | 0 | 0 | 0 | −16.88 | 0.57 | 0.92 |
| O75083 | WDR1 | 9\|14\|12 | 35 | 11.67 | 19\|19\|14 | 0 | 0 | 0 | 0 | 0 | −52.8 | 0.67 | 0.92 |
| O43252 | PAPSS1 | 3\|3\|4 | 10 | 3.33 | 2\|2\|3 | 0 | 0.01 | 0 | 0.01 | 0 | −6.63 | 1.43 | 0.83 |
| Q15149-7 | PLEC | 66\|60\|73 | 199 | 66.33 | 66\|62\|65 | 0 | 0 | 0 | 0 | 0 | −142.57 | 1.03 | 0.92 |
| P62913-2 | RPL11 | 3\|4\|3 | 10 | 3.33 | 3\|4\|4 | 0 | 0 | 0 | 0 | 0 | −11.36 | 0.91 | 0.89 |
| P53350 | PLK1 | 2\|3\|4 | 9 | 3 | 3\|3\|5 | 0 | 0 | 0 | 0 | 0 | −12.99 | 0.82 | 0.89 |
| P35251-2 | RFC1 | 3\|3\|3 | 9 | 3 | 5\|4\|4 | 0 | 0 | 0 | 0 | 0 | −13.76 | 0.69 | 0.92 |
| P54886 | ALDH18A1 | 12\|13\|16 | 41 | 13.67 | 21\|16\|18 | 0 | 0 | 0 | 0 | 0 | −50.95 | 0.75 | 0.92 |
| Q02978-2 | SLC25A11 | 4\|2\|4 | 10 | 3.33 | 3\|2\|2 | 0 | 0.01 | 0 | 0.01 | 0 | −8.1 | 1.43 | 0.82 |
| P52732 | KIF11 | 8\|9\|6 | 23 | 7.67 | 10\|11\|9 | 0 | 0 | 0 | 0 | 0 | −29.94 | 0.77 | 0.92 |
| P46781 | RPS9 | 14\|14\|15 | 43 | 14.33 | 14\|16\|17 | 0 | 0 | 0 | 0 | 0 | −37.71 | 0.91 | 0.92 |
| O94808 | GFPT2 | 2\|2\|2 | 6 | 2 | 3\|2\|3 | 0 | 0 | 0 | 0 | 0 | −9.29 | 0.75 | 0.92 |
| P11182 | DBT | 63\|65\|62 | 190 | 63.33 | 59\|65\|71 | 0 | 0 | 0 | 0 | 0 | −141.96 | 0.97 | 0.92 |
| Q99459 | CDC5L | 3\|0\|3 | 6 | 2 | 7\|5\|5 | 0 | 0 | 0 | 0 | 0 | −26.48 | 0.35 | 0.92 |
| O75376 | NCOR1 | 2\|0\|3 | 5 | 1.67 | 7\|7\|8 | 0 | 0 | 0 | 0 | 0 | −33.84 | 0.23 | 0.92 |
| P11216 | PYGB | 2\|2\|2 | 6 | 2 | 3\|3\|3 | 0 | 0 | 0 | 0 | 0 | −10.42 | 0.67 | 0.92 |
| Q16512 | PKN1 | 4\|5\|5 | 14 | 4.67 | 4\|5\|5 | 0 | 0 | 0 | 0 | 0 | −13.43 | 1 | 0.89 |
| P20020-5 | ATP281 | 2\|2\|0 | 4 | 1.33 | 0\|2\|4 | 0 | 0 | 0 | 0 | 0 | −10.2 | 0.67 | 0.92 |
| P20340-2 | RAB6A | 10\|12\|10 | 32 | 10.67 | 11\|11\|12 | 0 | 0 | 0 | 0 | 0 | −28.23 | 0.94 | 0.91 |
| P40939 | HADHA | 17\|14\|22 | 53 | 17.67 | 16\|14\|19 | 0 | 0 | 0 | 0 | 0 | −40.12 | 1.08 | 0.92 |
| P36915 | GNL1 | 2\|2\|3 | 7 | 2.33 | 3\|2\|2 | 0 | 0 | 0 | 0 | 0 | −8.1 | 1 | 0.86 |
| A0A0B4J2D | LOC- | 0\|2\|0 | 2 | 0.67 | 2\|0\|2 | 0 | 0.01 | 0 | 0.01 | 0 | −7.8 | 0.5 | 0.82 |
| | 102724023 | | | | | | | | | | | | |
| Q9UNF1-2 | MAGED2 | 3\|3\|5 | 11 | 3.67 | 4\|2\|3 | 0 | 0 | 0 | 0 | 0 | −8.99 | 1.22 | 0.85 |
| P31943 | HNRNPH1 | 43\|43\|41 | 127 | 42.33 | 44\|47\|54 | 0 | 0 | 0 | 0 | 0 | −114.1 | 0.88 | 0.92 |
| Q13356 | PPIL2 | 2\|2\|3 | 7 | 2.33 | 5\|4\|4 | 0 | 0 | 0 | 0 | 0 | −15.55 | 0.54 | 0.92 |
| Q8N1F7 | NUP93 | 4\|4\|5 | 13 | 4.33 | 4\|3\|6 | 0 | 0 | 0 | 0 | 0 | −12.24 | 1 | 0.89 |
| Q9BSV6 | TSEN34 | 4\|0\|3 | 7 | 2.33 | 3\|4\|0 | 0 | 0.01 | 0 | 0.01 | 0 | −11.76 | 1 | 0.83 |
| Q9Y316-2 | MEMO1 | 3\|0\|2 | 5 | 1.67 | 0\|2\|4 | 0 | 0.01 | 0 | 0.01 | 0 | −10.2 | 0.83 | 0.83 |
| P38646 | HSPA9 | 33\|28\|32 | 93 | 31 | 36\|42\|34 | 0 | 0 | 0 | 0 | 0 | −94.79 | 0.83 | 0.92 |
| Q9UMS4 | PRPF19 | 4\|2\|6 | 12 | 4 | 8\|9\|12 | 0 | 0 | 0 | 0 | 0 | −37.55 | 0.41 | 0.92 |
| P49916-4 | LIG3 | 0\|0\|2 | 2 | 0.67 | 2\|2\|0 | 0 | 0.01 | 0 | 0.01 | 0 | −7.8 | 0.5 | 0.82 |
| P60953 | CDC42 | 6\|7\|6 | 19 | 6.33 | 6\|6\|4 | 0 | 0 | 0 | 0 | 0 | −12.86 | 1.19 | 0.89 |
| Q9Y5B9 | SUPT16H | 12\|8\|11 | 31 | 10.33 | 12\|10\|9 | 0 | 0 | 0 | 0 | 0 | −27.73 | 1 | 0.91 |
| Q12986-2 | INFX1 | 3\|0\|2 | 5 | 1.67 | 2\|2\|3 | 0 | 0 | 0 | 0 | 0 | −11.93 | 0.71 | 0.86 |
| Q9GZT8-2 | INIF3L1 | 2\|3\|3 | 8 | 2.67 | 4\|4\|3 | 0 | 0 | 0 | 0 | 0 | −12.99 | 0.73 | 0.92 |
| O60610-2 | DIAPH1 | 4\|8\|3 | 15 | 5 | 6\|9\|8 | 0 | 0 | 0 | 0 | 0 | −26.78 | 0.65 | 0.91 |
| Q8IVF2-3 | AHNAK2 | 2\|5\|4 | 11 | 3.67 | 6\|4\|3 | 0 | 0 | 0 | 0 | 0 | −15.55 | 0.85 | 0.89 |
| Q9H0C8 | ILKAP | 2\|2\|2 | 6 | 2 | 2\|3\|3 | 0 | 0 | 0 | 0 | 0 | −9.29 | 0.75 | 0.92 |
| Q96151-2 | RCC1L | 0\|2\|0 | 2 | 0.67 | 0\|2\|2 | 0 | 0.01 | 0 | 0.01 | 0 | −7.8 | 0.5 | 0.82 |
| Q99460-2 | PSMD1 | 6\|6\|10 | 22 | 7.33 | 13\|9\|8 | 0 | 0 | 0 | 0 | 0 | −29.94 | 0.73 | 0.92 |
| Q13586 | STIM1 | 2\|2\|0 | 4 | 1.33 | 3\|3\|0 | 0 | 0 | 0 | 0 | 0 | −10.34 | 0.67 | 0.92 |
| Q86UR5-1 | RIMS1 | 2\|2\|0 | 4 | 1.33 | 3\|3\|2 | 0 | 0 | 0 | 0 | 0 | −13.39 | 0.5 | 0.92 |
| P30566 | ADSL | 3\|2\|2 | 7 | 2.33 | 2\|2\|3 | 0 | 0 | 0 | 0 | 0 | −8.1 | 1 | 0.86 |
| Q81X01-4 | SUGP2 | 7\|7\|5 | 19 | 6.33 | 24\|20\|24 | 0 | 0 | 0 | 0 | 0 | −84.41 | 0.28 | 0.92 |
| O95628-8 | CNOT4 | 0\|0\|2 | 2 | 0.67 | 2\|2\|0 | 0 | 0.01 | 0 | 0.01 | 0 | −7.8 | 0.5 | 0.82 |
| Q9NW13 | RBM28 | 4\|4\|3 | 11 | 3.67 | 5\|6\|7 | 0 | 0 | 0 | 0 | 0 | −20.1 | 0.61 | 0.92 |
| P38159 | RBMX | 16\|20\|21 | 57 | 19 | 34\|31\|32 | 0 | 0 | 0 | 0 | 0 | −98.01 | 0.59 | 0.92 |
| Q81WZ3-6 | ANKHD1 | 9\|9\|12 | 30 | 10 | 6\|12\|10 | 0 | 0 | 0 | 0 | 0 | −22.61 | 1.07 | 0.91 |
| Q9Y285 | FARSA | 3\|4\|4 | 11 | 3.67 | 3\|5\|5 | 0 | 0 | 0 | 0 | 0 | −13.76 | 0.85 | 0.92 |
| P43034 | PAFAH1B1 | 6\|8\|10 | 24 | 8 | 7\|4\|8 | 0 | 0 | 0 | 0 | 0 | −16.39 | 1.26 | 0.89 |
| Q9BRJ2 | MRPL45 | 4\|5\|4 | 13 | 4.33 | 4\|2\|4 | 0 | 0 | 0 | 0 | 0 | −8.71 | 1.3 | 0.86 |
| Q9BWM7 | SFXN3 | 3\|2\|3 | 8 | 2.67 | 3\|0\|4 | 0 | 0 | 0 | 0 | 0 | −7.93 | 1.14 | 0.85 |
| Q06124 | PTPN11 | 2\|3\|3 | 8 | 2.67 | 3\|4\|4 | 0 | 0 | 0 | 0 | 0 | −12.99 | 0.73 | 0.92 |
| P31939-2 | ATIC | 8\|9\|5 | 22 | 7.33 | 7\|7\|4 | 0 | 0 | 0 | 0 | 0 | −16.65 | 1.22 | 0.89 |
| Q9UHX1-4 | PUF60 | 9\|8\|10 | 27 | 9 | 6\|9\|6 | 0 | 0 | 0 | 0 | 0 | −15.83 | 1.29 | 0.9 |
| P13010 | XRCC5 | 46\|45\|45 | 136 | 45.33 | 45\|51\|49 | 0 | 0 | 0 | 0 | 0 | −107.91 | 0.94 | 0.92 |
| Q9UPQ9-1 | TNRC6B | 17\|12\|12 | 41 | 13.67 | 13\|15\|18 | 0 | 0 | 0 | 0 | 0 | −39.65 | 0.89 | 0.92 |
| O95340 | PAPSS2 | 8\|5\|6 | 19 | 6.33 | 4\|7\|6 | 0 | 0 | 0 | 0 | 0 | −15.5 | 1.12 | 0.89 |
| O95347 | SMC2 | 18\|18\|15 | 51 | 17 | 14\|13\|18 | 0 | 0 | 0 | 0 | 0 | −33.87 | 1.13 | 0.92 |
| Q8IZP0-3 | ABI1 | 12\|12\|13 | 37 | 12.33 | 11\|11\|9 | 0 | 0 | 0 | 0 | 0 | −21.79 | 1.19 | 0.91 |
| Q8IZP0-2 | ABI1 | 11\|10\|12 | 33 | 11 | 10\|10\|8 | 0 | 0 | 0 | 0 | 0 | −21.15 | 1.18 | 0.91 |
| Q99729-3 | HNRNPAB | 5\|4\|4 | 13 | 4.33 | 4\|4\|5 | 0 | 0 | 0 | 0 | 0 | −12.24 | 1 | 0.89 |
| Q9Y310 | RTCB | 15\|18\|8 | 41 | 13.67 | 15\|13\|13 | 0 | 0 | 0 | 0 | 0 | −40.39 | 1 | 0.91 |
| P37108 | SRP14 | 5\|5\|7 | 17 | 5.67 | 4\|4\|3 | 0 | 0 | 0 | 0 | 0 | −8.45 | 1.55 | 0.84 |
| P62829 | RPL23 | 15\|13\|13 | 41 | 13.67 | 14\|13\|14 | 0 | 0 | 0 | 0 | 0 | −32.08 | 3 | 0.92 |
| P62820 | RABIA | 18\|14\|16 | 48 | 16 | 15\|18\|16 | 0 | 0 | 0 | 0 | 0 | −40.12 | 0.98 | 0.92 |
| P62826 | RAN | 14\|15\|19 | 48 | 16 | 18\|15\|18 | 0 | 0 | 0 | 0 | 0 | −42.54 | 0.94 | 0.92 |
| Q9Y4W6 | AFG3L2 | 3\|3\|3 | 9 | 3 | 3\|3\|6 | 0 | 0 | 0 | 0 | 0 | −12.5 | 0.75 | 0.92 |
| Q08378-2 | GOLGA3 | 0\|3\|2 | 5 | 1.67 | 6\|4\|0 | 0 | 0 | 0 | 0 | 0 | −15.87 | 0.5 | 0.92 |
| Q9NXH9-2 | TRMT1 | 8\|11\|8 | 27 | 9 | 8\|10\|10 | 0 | 0 | 0 | 0 | 0 | −24.09 | 0.96 | 0.91 |
| O95299 | NDUFA10 | 5\|0\|6 | 11 | 3.67 | 3\|3\|3 | 0 | 0.01 | 0 | 0.01 | 0 | −14.75 | 1.22 | 0.82 |
| Q92615 | LARP4B | 5\|9\|6 | 20 | 6.67 | 6\|7\|6 | 0 | 0 | 0 | 0 | 0 | −17.88 | 1.05 | 0.9 |
| Q99661 | KIF2C | 4\|5\|6 | 15 | 5 | 6\|6\|5 | 0 | 0 | 0 | 0 | 0 | −17.06 | 0.88 | 0.9 |
| Q9Y4E8 | USP15 | 8\|5\|5 | 18 | 6 | 4\|4\|7 | 0 | 0 | 0 | 0 | 0 | −13.14 | 1.2 | 0.88 |
| P43490 | NAMPT | 20\|17\|20 | 57 | 19 | 18\|17\|19 | 0 | 0 | 0 | 0 | 0 | −41.54 | 1.06 | 0.92 |
| Q9BZK7 | TBL1XR1 | 2\|2\|2 | 6 | 2 | 4\|2\|5 | 0 | 0 | 0 | 0 | 0 | −12.99 | 0.55 | 0.92 |
| Q81YWS | RNF168 | 0\|0\|2 | 2 | 0.67 | 2\|3\|0 | 0 | 0 | 0 | 0 | 0 | −9.13 | 0.4 | 0.84 |
| P13674-3 | P4HA1 | 3\|2\|3 | 8 | 2.67 | 2\|3\|3 | 0 | 0 | 0 | 0 | 0 | −9.29 | 1 | 0.86 |
| P46063 | RECOL | 6\|6\|6 | 18 | 6 | 4\|4\|2 | 0 | 0 | 0 | 0 | 0 | −5.91 | 1.8 | 0.83 |
| O75964 | ATPSL | 2\|0\|2 | 4 | 1.33 | 2\|3\|0 | 0 | 0 | 0 | 0 | 0 | −9.13 | 0.8 | 0.83 |
| P42765 | ACAA2 | 69\|10 | 25 | 8.33 | 3\|6\|9 | 0 | 0 | 0 | 0 | 0 | −15.11 | 1.39 | 0.88 |
| P42766 | RPL35 | 4\|2\|2 | 8 | 2.67 | 5\|3\|3 | 0 | 0 | 0 | 0 | 0 | −12.99 | 0.73 | 0.89 |
| P22087 | FBL | 5\|5\|4 | 14 | 4.67 | 6\|5\|6 | 0 | 0 | 0 | 0 | 0 | −17.06 | 0.82 | 0.92 |
| P68371 | TUBB4B | 14\|112\|11 | 345 | 115 | 14\|111\|10 | 0 | 0 | 0 | 0 | 0 | −232.7 | 1.03 | 0.92 |
| Q9UHD1 | CHORDC1 | 12\|11\|14 | 37 | 12.33 | 14\|14\|9 | 0 | 0 | 0 | 0 | 0 | −30.3 | 1 | 0.91 |
| Q96GX5-2 | MASTL | 0\|2\|0 | 2 | 0.67 | 0\|3\|5 | 0 | 0 | 0 | 0 | 0 | −13.16 | 0.25 | 0.92 |
| P53618 | COPB1 | 10\|14\|12 | 36 | 12 | 12\|13\|15 | 0 | 0 | 0 | 0 | 0 | −35.55 | 0.9 | 0.92 |
| P62318-2 | SNRPD3 | 2\|2\|2 | 6 | 2 | 2\|3\|2 | 0 | 0 | 0 | 0 | 0 | −8.1 | 0.86 | 0.92 |
| P18124 | RPL7 | 17\|18\|16 | 51 | 17 | 24\|13\|15 | 0 | 0 | 0 | 0 | 0 | −40.43 | 0.98 | 0.92 |
| P50991 | CCT4 | 23\|20\|28 | 71 | 23.67 | 25\|24\|31 | 0 | 0 | 0 | 0 | 0 | −68.46 | 0.89 | 0.92 |
| P24666 | ACP1 | 5\|5\|5 | 15 | 5 | 3\|4\|4 | 0 | 0 | 0 | 0 | 0 | −8.45 | 1.36 | 0.87 |
| Q9GZS3 | WDR61 | 3\|4\|3 | 10 | 3.33 | 2\|4\|3 | 0 | 0 | 0 | 0 | 0 | −8.99 | 1.11 | 0.87 |
| Q96HC4 | PDLIM5 | 6\|8\|9 | 23 | 7.67 | 7\|7\|5 | 0 | 0 | 0 | 0 | 0 | −16.39 | 1.21 | 0.9 |
| 095433 | AHSA1 | 21\|27\|21 | 69 | 23 | 23\|19\|22 | 0 | 0 | 0 | 0 | 0 | −47.49 | 1.08 | 0.92 |
| P49327 | FASN | 66\|68\|64 | 198 | 66 | 65\|73\|73 | 0 | 0 | 0 | 0 | 0 | −158.04 | 0.94 | 0.92 |
| Q8NG31-2 | KNL1 | 2\|0\|0 | 2 | 0.67 | 0\|3\|4 | 0 | 0 | 0 | 0 | 0 | −11.76 | 0.29 | 0.92 |
| P00387-2 | CYB5R3 | 4\|4\|4 | 12 | 4 | 5\|5\|4 | 0 | 0 | 0 | 0 | 0 | −13.43 | 0.86 | 0.92 |
| P14618 | PKM | 75\|67\|74 | 216 | 72 | 80\|69\|81 | 0 | 0 | 0 | 0 | 0 | −176.31 | 0.94 | 0.92 |
| P25685 | DNAIB1 | 9\|8\|4 | 21 | 7 | 8\|11\|5 | 0 | 0 | 0 | 0 | 0 | −26.03 | 0.88 | 0.91 |
| P42167 | TMPO | 4\|0\|3 | 7 | 2.33 | 7\|6\|8 | 0 | 0 | 0 | 0 | 0 | −32.34 | 0.33 | 0.92 |
| P42166 | TMPO | 2\|0\|3 | 5 | 1.67 | 6\|5\|7 | 0 | 0 | 0 | 0 | 0 | −27.91 | 0.28 | 0.92 |
| Q14019 | COTL1 | 2\|2\|0 | 4 | 1.33 | 2\|2\|4 | 0 | 0 | 0 | 0 | 0 | −13.39 | 0.5 | 0.92 |
| P62195-2 | PSMC5 | 18\|17\|16 | 51 | 17 | 17\|15\|14 | 0 | 0 | 0 | 0 | 0 | −33.6 | 1.11 | 0.92 |
| Q8NFH3 | NUP43 | 4\|4\|4 | 12 | 4 | 5\|3\|3 | 0 | 0 | 0 | 0 | 0 | −9.88 | 1.09 | 0.88 |
| P18583-5 | SON | 5\|4\|4 | 13 | 4.33 | 12\|14\|8 | 0 | 0 | 0 | 0 | 0 | −39.44 | 0.38 | 0.92 |
| Q9NYK5 | MRPL39 | 5\|4\|0 | 9 | 3 | 4\|5\|6 | 0 | 0 | 0 | 0 | 0 | −23.5 | 0.6 | 0.92 |
| Q96PK6 | RBM14 | 24\|23\|24 | 71 | 23.67 | 23\|24\|23 | 0 | 0 | 0 | 0 | 0 | −51.64 | 1.01 | 0.92 |
| Q9BQE3 | TUBA1C | 81\|68\|73 | 222 | 74 | 71\|68\|73 | 0 | 0 | 0 | 0 | 0 | −153.22 | 1.05 | 0.92 |
| Q8N1G2 | CMTR1 | 2\|2\|2 | 6 | 2 | 5\|4\|5 | 0 | 0 | 0 | 0 | 0 | −16.88 | 0.43 | 0.92 |
| P41091 | EIF253 | 19\|17\|16 | 52 | 17.33 | 17\|22\|18 | C | 0 | 0 | 0 | 0 | −46.67 | 0.91 | 0.92 |
| O60231 | DHX16 | 0\|2\|3 | 5 | 1.67 | 5\|4\|4 | 0 | 0 | 0 | 0 | 0 | −20.61 | 0.38 | 0.92 |
| Q12906-7 | ILF3 | 20\|20\|28 | 68 | 22.67 | 29\|36\|30 | 0 | 0 | 0 | 0 | 0 | −87.49 | 0.72 | 0.92 |
| Q96KK5 | HIST1H2AH | 5\|4\|6 | 15 | 5 | 11\|9\|9 | 0 | 0 | 0 | 0 | 0 | −32.66 | 0.52 | 0.92 |
| Q9BQ39 | DDX50 | 3\|3\|3 | 9 | 3 | 4\|8\|5 | 0 | 0 | 0 | 0 | 0 | −18.8 | 0.53 | 0.92 |
| Q9NYY8 | FASTKD2 | 3\|6\|3 | 12 | 4 | 5\|5\|3 | 0 | 0 | 0 | 0 | 0 | −13.76 | 0.92 | 0.88 |
| P68366-2 | TUBA4A | 72\|61\|67 | 200 | 66.67 | 66\|67\|69 | 0 | 0 | 0 | 0 | 0 | −151.83 | 0.99 | 0.92 |
| Q9NS69 | TOMM22 | 3\|5\|3 | 11 | 3.67 | 3\|2\|7 | 0 | 0 | 0 | 0 | 0 | −12.32 | 0.92 | 0.88 |
| Q16658 | FSCN1 | 14\|9\|10 | 33 | 11 | 12\|10\|21 | 0 | 0 | 0 | 0 | 0 | −40.77 | 0.77 | 0.92 |
| Q9ULD2-2 | MTUS1 | 0\|3\|2 | 5 | 1.67 | 4\|4\|3 | 0 | 0 | 0 | 0 | 0 | −17.71 | 0.45 | 0.92 |
| P13489 | RNH3 | 9\|11\|9 | 29 | 9.67 | 7\|8\|9 | 0 | 0 | 0 | 0 | 0 | −17.91 | 1.21 | 0.9 |
| Q14839 | CHD4 | 6\|7\|8 | 21 | 7 | 13\|16\|14 | 0 | 0 | 0 | 0 | 0 | −47.13 | 0.49 | 0.92 |
| P50402 | EMD | 9\|7\|8 | 24 | 8 | 7\|7\|7 | 0 | 0 | 0 | 0 | 0 | −17.28 | 1.14 | 0.9 |
| Q96GA3 | LTV1 | 9\|7\|7 | 23 | 7.67 | 10\|8\|11 | 0 | 0 | 0 | 0 | 0 | −26.92 | 0.79 | 0.92 |
| P31150 | GDI1 | 4\|6\|6 | 16 | 5.33 | 7\|5\|5 | 0 | 0 | 0 | 0 | 0 | −17.06 | 0.94 | 0.9 |
| Q96EN8 | MOCOS | 8\|5\|6 | 19 | 6.33 | 3\|5\|4 | 0 | 0 | 0 | 0 | 0 | −9.61 | 1.58 | 0.84 |
| P48444 | ARCN1 | 16\|18\|16 | 50 | 16.67 | 16\|16\|15 | 0 | 0 | 0 | 0 | 0 | −34.77 | 1.06 | 0.92 |
| Q9Y4E1-3 | WASHC2C | 2\|3\|0 | 5 | 1.67 | 2\|2\|4 | 0 | 0 | 0 | 0 | 0 | −13.39 | 0.63 | 0.87 |
| Q15233 | NONO | 42\|46\|41 | 129 | 43 | 48\|45\|47 | 0 | 0 | 0 | 0 | 0 | −108.04 | 0.92 | 0.92 |
| P29218 | IMPA1 | 5\|5\|3 | 13 | 4.33 | 5\|4\|6 | 0 | 0 | 0 | 0 | 0 | −16.23 | 0.87 | 0.92 |
| P21796 | VDAC1 | 4\|4\|2 | 10 | 3.33 | 3\|2\|2 | 0 | 0.01 | 0 | 0.01 | 0 | −8.1 | 1.43 | 0.82 |
| P14923 | JUP | 2\|2\|2 | 6 | 2 | 3\|4\|0 | 0 | 0 | 0 | 0 | 0 | −7.93 | 0.86 | 0.92 |
| P35606-2 | COPB2 | 19\|18\|25 | 62 | 20.67 | 14\|22\|14 | 0 | 0 | 0 | 0 | 0 | −35.38 | 1.24 | 0.91 |
| Q9UQ35 | SRRM2 | 5\|5\|9 | 19 | 6.33 | 18\|13\|15 | 0 | 0 | 0 | 0 | 0 | −53.55 | 0.41 | 0.92 |
| O00458 | IFRD1 | 6\|5\|5 | 16 | 5.33 | 4\|4\|4 | 0 | 0 | 0 | 0 | 0 | −9.61 | 1.33 | 0.87 |
| P18463 | HLA-B | 4\|3\|4 | 11 | 3.67 | 4\|3\|3 | 0 | 0 | 0 | 0 | 0 | −10.17 | 1.1 | 0.87 |
| Q9P2R7-2 | SUCLA2 | 5\|7\|5 | 17 | 5.67 | 10\|7\|6 | 0 | 0 | 0 | 0 | 0 | −22.85 | 0.74 | 0.92 |
| P16615 | ATP2A2 | 20\|18\|20 | 58 | 19.33 | 14\|15\|21 | 0 | 0 | 0 | 0 | 0 | −35.4 | 1.16 | 0.92 |
| Q9BRS2 | RIOK1 | 3\|3\|0 | 6 | 2 | 4\|4\|2 | 0 | 0 | 0 | 0 | 0 | −16.24 | 0.6 | 0.92 |
| Q9P0J0 | NDUFA13 | 2\|0\|0 | 2 | 0.67 | 0\|2\|2 | 0 | 0.01 | 0 | 0.01 | 0 | −7.8 | 0.5 | 0.82 |
| Q12972 | PPP1R8 | 3\|3\|3 | 9 | 3 | 5\|3\|5 | 0 | 0 | 0 | 0 | 0 | −13.76 | 0.69 | 0.92 |
| P29144 | TPP2 | 10\|10\|10 | 30 | 10 | 6\|9\|12 | 0 | 0 | 0 | 0 | 0 | −19.98 | 1.11 | 0.91 |
| P19525 | EIF2AK2 | 7\|10\|6 | 23 | 7.67 | 6\|9\|9 | 0 | 0 | 0 | 0 | 0 | −22.37 | 0.96 | 0.91 |
| Q9UKK9 | NUDTS | 5\|5\|6 | 16 | 5.33 | 6\|7\|6 | 0 | 0 | 0 | 0 | 0 | −17.88 | 0.84 | 0.92 |
| Q8IVT2 | MISP | 18\|18\|20 | 56 | 18.67 | 24\|26\|24 | 0 | 0 | 0 | 0 | 0 | −64.38 | 0.76 | 0.92 |
| Q00839-2 | HNRNPU | 29\|31\|29 | 89 | 29.67 | 33\|31\|29 | 0 | 0 | 0 | 0 | 0 | −69.98 | 0.96 | 0.92 |
| P14324-2 | FDPS | 4\|3\|4 | 11 | 3.67 | 6\|5\|5 | 0 | 0 | 0 | 0 | 0 | −17.53 | 0.69 | 0.92 |
| Q00610-2 | CLTC | 25\|24\|26 | 75 | 25 | 28\|26\|29 | 0 | 0 | 0 | 0 | 0 | −65.63 | 0.9 | 0.92 |
| O15355 | PPM1G | 2\|2\|2 | 6 | 2 | 3\|2\|2 | 0 | 0 | 0 | 0 | 0 | −8.1 | 0.86 | 0.92 |
| Q99832 | CCT7 | 24\|21\|22 | 67 | 22.33 | 24\|29\|28 | 0 | 0 | 0 | 0 | 0 | −68.01 | 0.83 | 0.92 |
| P04150-2 | NR3C1 | 3\|4\|2 | 9 | 3 | 2\|2\|3 | 0 | 0.01 | 0 | 0.01 | 0 | −8.1 | 1.29 | 0.83 |
| P01113 | NRAS | 6\|6\|6 | 18 | 6 | 4\|3\|6 | 0 | 0 | 0 | 0 | 0 | −9.36 | 1.38 | 0.88 |
| Q9H2P0 | ADNP | 14\|11\|14 | 39 | 13 | 19\|18\|15 | 0 | 0 | 0 | 0 | 0 | −48.94 | 0.75 | 0.92 |
| P61978-3 | HNRNPK | 38\|36\|31 | 105 | 35 | 35\|43\|39 | 0 | 0 | 0 | 0 | 0 | −95.96 | 0.9 | 0.92 |
| Q13111 | CHAFIA | 9\|8\|11 | 28 | 9.33 | 9\|8\|11 | 0 | 0 | 0 | 0 | 0 | −24.09 | 3 | 0.91 |
| Q8N543 | OGFOD1 | 2\|0\|0 | 2 | 0.67 | 2\|0\|2 | 0 | 0.01 | 0 | 0.01 | 0 | −7.8 | 0.5 | 0.82 |
| P35527 | KRT9 | 44\|33\|52 | 129 | 43 | 50\|39\|35 | 0 | 0 | 0 | 0 | 0 | −101.22 | 1.04 | 0.92 |
| P61978 | HNRNPK | 38\|38\|33 | 109 | 36.33 | 35\|44\|39 | 0 | 0 | 0 | 0 | 0 | −93.94 | 0.92 | 0.92 |
| Q14247 | CTTN | 12\|12\|14 | 38 | 12.67 | 14\|11\|14 | 0 | 0 | 0 | 0 | 0 | −31.17 | 0.97 | 0.92 |
| Q14244 | MAP7 | 40\|32\|39 | 111 | 37 | 31\|27\|37 | 0 | 0 | 0 | 0 | 0 | −67.96 | 1.17 | 0.92 |
| Q14240 | EIF4A2 | 23\|22\|19 | 64 | 21.33 | 21\|29\|27 | 0 | 0 | 0 | 0 | 0 | −66.42 | 0.83 | 0.92 |
| Q81WVX8 | CHERP | 2\|0\|2 | 4 | 1.33 | 6\|5\|7 | 0 | 0 | 0 | 0 | 0 | −42.35 | 0.14 | 0.92 |
| P24928 | POLRZA | 6\|7\|6 | 19 | 6.33 | 5\|5\|7 | 0 | 0 | 0 | 0 | 0 | −14.03 | 1.12 | 0.9 |
| Q5JSZS | PRRC2B | 8\|8\|7 | 23 | 7.67 | 10\|7\|9 | 0 | 0 | 0 | 0 | 0 | −23.23 | 0.88 | 0.92 |
| Q13200 | PSMD2 | 18\|14\|19 | 51 | 17 | 13\|16\|17 | 0 | 0 | 0 | 0 | 0 | −36.52 | 1.11 | 0.92 |
| P63000 | RAC1 | 6\|6\|7 | 19 | 6.33 | 9\|8\|6 | 0 | 0 | 0 | 0 | 0 | −21.17 | 0.83 | 0.92 |
| Q8N163 | CCAR2 | 20\|18\|22 | 60 | 20 | 19\|25\|26 | 0 | 0 | 0 | 0 | 0 | −59.39 | 0.86 | 0.92 |
| Tx0002 | Tx0002 | 71\|632\|65 | 1957 | 652.33 | 42\|671\|68 | 0 | 0 | 0 | 0 | 0 | #NAME? | 0.98 | 0.92 |
| Q15758 | SLC1A5 | 5\|5\|0 | 10 | 3.33 | 4\|4\|5 | 0 | 0 | 0 | 0 | 0 | −20.61 | 0.77 | 0.89 |
| Q8WX93-3 | PALLD | 8\|5\|11 | 24 | 8 | 8\|7\|8 | 0 | 0 | 0 | 0 | 0 | −22.85 | 1.04 | 0.9 |
| Q9NXCS | MIOS | 0\|2\|0 | 2 | 0.67 | 2\|2\|0 | 0 | 0.01 | 0 | 0.01 | 0 | −7.8 | 0.5 | 0.82 |
| O14617-3 | AP3D1 | 3\|4\|3 | 10 | 3.33 | 2\|2\|5 | 0 | 0 | 0 | 0 | 0 | −8.99 | 1.11 | 0.87 |
| P15170-2 | GSPT1 | 4\|3\|5 | 12 | 4 | 6\|8\|7 | 0 | 0 | 0 | 0 | 0 | −24.06 | 0.57 | 0.92 |
| O15427 | SLC16A3 | 3\|4\|2 | 9 | 3 | 3\|2\|4 | 0 | 0 | 0 | 0 | 0 | −10.4 | 1 | 0.87 |
| Q9NWB6 | ARGLU1 | 3\|2\|2 | 7 | 2.33 | 4\|3\|3 | 0 | 0 | 0 | 0 | 0 | −11.71 | 0.7 | 0.92 |
| Q16795 | NDUFA9 | 4\|3\|2 | 9 | 3 | 5\|5\|4 | 0 | 0 | 0 | 0 | 0 | −16.88 | 0.64 | 0.92 |
| P62280 | RPS11 | 14\|13\|17 | 44 | 14.67 | 17\|11\|13 | 0 | 0 | 0 | 0 | 0 | −32.08 | 1.07 | 0.91 |
| P27816-4 | MAP4 | 11\|11\|12 | 34 | 11.33 | 23\|14\|15 | 0 | 0 | 0 | 0 | 0 | −48.88 | 0.65 | 0.92 |
| Q9BTX1-6 | NDC1 | 2\|0\|0 | 2 | 0.67 | 3\|3\|0 | 0 | 0 | 0 | 0 | 0 | −10.34 | 0.33 | 0.92 |
| Q6ZW31-2 | SYDE1 | 0\|2\|0 | 2 | 0.67 | 2\|4\|0 | 0 | 0 | 0 | 0 | 0 | −10.2 | 0.33 | 0.92 |
| Q96RP9 | GFM1 | 6\|8\|8 | 22 | 7.33 | 8\|7\|9 | 0 | 0 | 0 | 0 | 0 | −22.37 | 0.92 | 0.92 |
| O60488-2 | ACSL4 | 4\|2\|3 | 9 | 3 | 3\|4\|2 | 0 | 0 | 0 | 0 | 0 | −10.42 | 1 | 0.87 |
| O95235-2 | KIF20A | 2\|4\|3 | 9 | 3 | 4\|8\|6 | 0 | 0 | 0 | 0 | 0 | −22.21 | 0.5 | 0.92 |
| P61158 | ACTR3 | 5\|7\|4 | 16 | 5.33 | 11\|7\|9 | 0 | 0 | 0 | 0 | 0 | −29.96 | 0.59 | 0.92 |
| P56134-4 | ATPSJ2 | 4\|4\|4 | 12 | 4 | 5\|4\|3 | 0 | 0 | 0 | 0 | 0 | −11.06 | 1 | 0.92 |
| O95602 | POLRIA | 13\|4\|5 | 22 | 7.33 | 7\|7\|7 | 0 | 0 | 0 | 0 | 0 | −22.08 | 1.05 | 0.88 |
| Q9H832 | UBE2Z | 0\|0\|3 | 3 | 3 | 4\|2\|2 | 0 | 0 | 0 | 0 | 0 | −13.39 | 0.38 | 0.87 |
| O94776 | MTA2 | 18\|19\|17 | 54 | 18 | 16\|15\|16 | 0 | 0 | 0 | 0 | 0 | −33.33 | 1.15 | 0.92 |
| Q15382 | RHEB | 5\|6\|4 | 15 | 5 | 6\|4\|3 | 0 | 0 | 0 | 0 | 0 | −12.24 | 1.15 | 0.88 |
| P62191 | PSMC1 | 11\|6\|12 | 29 | 9.67 | 11\|12\|11 | 0 | 0 | 0 | 0 | 0 | −35.15 | 0.85 | 0.91 |
| P11413 | G6PD | 10\|10\|11 | 31 | 10.33 | 11\|12\|11 | 0 | 0 | 0 | 0 | 0 | −28.23 | 0.91 | 0.92 |
| P12036-2 | NEFH | 3\|2\|3 | 8 | 2.67 | 3\|2\|2 | 0 | 0 | 0 | 0 | 0 | −8.1 | 1.14 | 0.85 |
| P11142 | HSPA8 | 50\|55\|52 | 157 | 52.33 | 62\|51\|54 | 0 | 0 | 0 | 0 | 0 | −126.65 | 0.94 | 0.92 |
| Q9BVP2-2 | GNL3 | 2\|5\|3 | 10 | 3.33 | 6\|7\|5 | 0 | 0 | 0 | 0 | 0 | −22.21 | 0.56 | 0.92 |
| Q13501-2 | SQSTM1 | 11\|10\|11 | 32 | 10.67 | 9\|7\|7 | 0 | 0 | 0 | 0 | 0 | −15.32 | 1.39 | 0.9 |
| P30492 | HLA-B | 4\|3\|4 | 1.1 | 3.67 | 4\|3\|3 | 0 | 0 | 0 | 0 | 0 | −10.17 | 1.1 | 0.87 |
| P30493 | HLA-B | 4\|3\|4 | 11 | 3.67 | 4\|3\|3 | 0 | 0 | 0 | 0 | 0 | −10.17 | 1.1 | 0.87 |
| P61081 | UBE2M | 8\|6\|8 | 22 | 7.33 | 8\|6\|6 | 0 | 0 | 0 | 0 | 0 | −17.57 | 1.1 | 0.9 |
| P62266 | RPS23 | 2\|2\|2 | 6 | 2 | 4\|3\|3 | 0 | 0 | 0 | 0 | 0 | −11.71 | 0.6 | 0.92 |
| P62263 | RPS14 | 19\|20\|18 | 57 | 19 | 20\|16\|18 | 0 | 0 | 0 | 0 | 0 | −40.1 | 1.06 | 0.92 |
| Q16513-3 | PKN2 | 0\|0\|2 | 2 | 0.67 | 2\|3\|0 | 0 | 0 | 0 | 0 | 0 | −9.13 | 0.4 | 0.84 |
| P62269 | RPS18 | 6\|6\|5 | 17 | 5.67 | 4\|3\|6 | 0 | 0 | 0 | 0 | 0 | −10.78 | 1.31 | 0.88 |
| Q9BR76 | CORO1B | 3\|2\|4 | 9 | 3 | 3\|3\|2 | 0 | 0 | 0 | 0 | 0 | −9.29 | 1.12 | 0.85 |
| O08945 | SSRP1 | 3\|4\|5 | 1.2 | 4 | 9\|5\|4 | 0 | 0 | 0 | 0 | 0 | −20.13 | 0.67 | 0.92 |
| P20339-2 | RAB5A | 7\|4\|7 | 18 | 6 | 4\|6\|7 | 0 | 0 | 0 | 0 | 0 | −17.06 | 1.06 | 0.9 |
| Q96C36 | PYCR2 | 2\|3\|0 | 5 | 1.67 | 3\|2\|3 | 0 | 0 | 0 | 0 | 0 | −13.39 | 0.63 | 0.87 |
| P07195 | LDHB | 10\|10\|11 | 31 | 10.33 | 11\|12\|12 | 0 | 0 | 0 | 0 | 0 | −29.43 | 0.89 | 0.92 |
| O00330 | PDHX | 2\|3\|3 | 8 | 2.67 | 4\|0\|3 | 0 | 0 | 0 | 0 | 0 | −7.93 | 1.14 | 0.85 |
| P53992 | SEC24C | 5\|7\|10 | 22 | 7.33 | 5\|6\|9 | 0 | 0 | 0 | 0 | 0 | −19.11 | 1.1 | 0.9 |
| Q9NTJ3 | SMC4 | 12\|9\|15 | 36 | 12 | 10\|12\|7 | 0 | 0 | 0 | 0 | 0 | −23.79 | 1.24 | 0.9 |
| O95163 | IKBKAP | 4\|7\|4 | 15 | 5 | 3\|4\|3 | 0 | 0.01 | 0 | 0.01 | 0 | −8.71 | 1.5 | 0.82 |
| Q9NXV6 | CDKN2AIP | 5\|5\|6 | 16 | 5.33 | 2\|7\|6 | 0 | 0 | 0 | 0 | 0 | −13.07 | 1.07 | 0.89 |
| P13639 | EEF2 | 87\|80\|85 | 252 | 84 | 85\|75\|83 | 0 | 0 | 0 | 0 | 0 | −172.2 | 1.04 | 0.92 |
| P52907 | CAPZA1 | 7\|7\|4 | 18 | 6 | 6\|6\|4 | 0 | 0 | 0 | 0 | 0 | −15.82 | 1.12 | 0.89 |
| P62841 | RPS15 | 7\|4\|10 | 21 | 7 | 5\|6\|7 | 0 | 0 | 0 | 0 | 0 | −18.27 | 1.17 | 0.88 |
| Q9GZL7 | WDR12 | 6\|5\|6 | 17 | 5.67 | 4\|8\|7 | 0 | 0 | 0 | 0 | 0 | −17.88 | 0.89 | 0.92 |
| Q86UV5-2 | USP48 | 2\|0\|0 | 2 | 0.67 | 2\|2\|2 | 0 | 0 | 0 | 0 | 0 | −10.44 | 0.33 | 0.92 |
| P15880 | RPS2 | 11\|14\|14 | 39 | 13 | 11\|12\|12 | 0 | 0 | 0 | 0 | 0 | −27.93 | 1.11 | 0.91 |
| Q9BXS5 | AP1M1 | 5\|8\|7 | 20 | 6.67 | 6\|8\|8 | 0 | 0 | 0 | 0 | 0 | −21.58 | 0.91 | 0.91 |
| Q86XP3-2 | DDX42 | 0\|2\|0 | 2 | 0.67 | 2\|2\|0 | 0 | 0.01 | 0 | 0.01 | 0 | −7.8 | 0.5 | 0.82 |
| P55809 | OXCT1 | 13\|14\|13 | 40 | 13.33 | 13\|18\|17 | 0 | 0 | 0 | 0 | 0 | −40.47 | 0.83 | 0.92 |
| P53985 | SLC16A1 | 5\|6\|7 | 18 | 6 | 5\|4\|5 | 0 | 0 | 0 | 0 | 0 | −11.96 | 1.29 | 0.88 |
| P08195-2 | SLC3A2 | 21\|19\|15 | 55 | 18.33 | 19\|23\|21 | 0 | 0 | 0 | 0 | 0 | −55.75 | 0.87 | 0.92 |
| O99829 | CPNE1 | 6\|5\|6 | 17 | 5.67 | 9\|9\|10 | 0 | 0 | 0 | 0 | 0 | −29.27 | 0.61 | 0.92 |
| P08243-2 | ASNS | 10\|9\|12 | 31 | 10.33 | 13\|12\|19 | 0 | 0 | 0 | 0 | 0 | −42.38 | 0.7 | 0.92 |
| P78527 | PRKDC | 23\|15\|26 | 64 | 21.33 | 16\|20\|26 | 0 | 0 | 0 | 0 | 0 | −54.51 | 1.03 | 0.92 |
| Q92747 | ARPCIA | 3\|2\|0 | 5 | 1.67 | 3\|2\|3 | 0 | 0 | 0 | 0 | 0 | −13.39 | 0.63 | 0.87 |
| P11908 | PRPS2 | 5\|7\|7 | 19 | 6.33 | 4\|7\|8 | 0 | 0 | 0 | 0 | 0 | −17.88 | 1 | 0.9 |
| Q6ULP2-8 | \|AFTPH | 5\|3\|4 | 12 | 4 | 6\|7\|6 | 0 | 0 | 0 | 0 | 0 | −21.43 | 0.63 | 0.92 |
| Q99873-2 | PRMT1 | 7\|4\|5 | 16 | 5.33 | 9\|5\|3 | 0 | 0 | 0 | 0 | 0 | −16.94 | 0.94 | 0.9 |
| P31153 | MAT2A | 8\|10\|9 | 27 | 9 | 9\|10\|8 | 0 | 0 | 0 | 0 | 0 | −22.87 | 1 | 0.91 |
| P52565 | ARHGDIA | 10\|9\|11 | 30 | 10 | 8\|11\|10 | 0 | 0 | 0 | 0 | 0 | −23.79 | 1.03 | 0.91 |
| P27448-8 | MARK3 | 4\|0\|2 | 6 | 2 | 5\|4\|3 | 0 | 0 | 0 | 0 | 0 | −19.11 | 0.5 | 0.92 |
| P57088 | TMEM33 | 4\|3\|0 | 7 | 2.33 | 4\|5\|2 | 0 | 0 | 0 | 0 | 0 | −17.71 | 0.64 | 0.89 |
| P61981 | YWHAG | 9\|7\|11 | 27 | 9 | 11\|11\|16 | 0 | 0 | 0 | 0 | 0 | −38.42 | 0.71 | 0.92 |
| Q9UM54-6 | MYO6 | 15\|11\|19 | 45 | 15 | 28\|29\|28 | 0 | 0 | 0 | 0 | 0 | −92.84 | 0.53 | 0.92 |
| Q5VTR2 | RNF20 | 3\|3\|3 | 9 | 3 | 6\|5\|8 | 0 | 0 | 0 | 0 | 0 | −21.43 | 0.47 | 0.92 |
| Q9UJS0 | SLC25A13 | 13\|13\|16 | 42 | 14 | 12\|14\|16 | 0 | 0 | 0 | 0 | 0 | −33.25 | 1 | 0.92 |
| Q96GD4-4 | AURKB | 0\|0\|3 | 3 | 1 | 3\|0\|2 | 0 | 0.01 | 0 | 0.01 | 0 | −9.13 | 0.6 | 0.81 |
| Q02543 | RPL18A | 8\|8\|7 | 23 | 7.67 | 10\|6\|9 | 0 | 0 | 0 | 0 | 0 | −22.02 | 0.92 | 0.92 |
| Q05639 | EEF1A2 | 25\|23\|22 | 70 | 23.33 | 27\|25\|25 | 0 | 0 | 0 | 0 | 0 | −61.5 | 0.91 | 0.92 |
| Q6P2E9 | EDC4 | 5\|2\|3 | 10 | 3.33 | 3\|4\|4 | 0 | 0 | 0 | 0 | 0 | −12.99 | 0.91 | 0.88 |
| Q99567 | NUP88 | 5\|6\|9 | 20 | 6.67 | 5\|9\|9 | 0 | 0 | 0 | 0 | 0 | −22.85 | 0.87 | 0.91 |
| P49189 | ALDH9A1 | 7\|3\|4 | 14 | 4.67 | 8\|6\|8 | 0 | 0 | 0 | 0 | 0 | −25.42 | 0.64 | 0.92 |
| Q9Y570 | PPME1 | 2\|3\|0 | 5 | 1.67 | 3\|0\|4 | 0 | 0 | 0 | 0 | 0 | −11.76 | 0.71 | 0.85 |
| P39656-3 | DDOST | 0\|3\|3 | 6 | 2 | 3\|2\|2 | 0 | 0 | 0 | 0 | 0 | −11.93 | 0.86 | 0.85 |
| Q9NUQ8 | ABCF3 | 4\|3\|4 | 11 | 3.67 | 3\|3\|4 | 0 | 0 | 0 | 0 | 0 | −10.17 | 1.1 | 0.87 |
| P33176 | KIFSB | 15\|18\|24 | 57 | 19 | 20\|17\|18 | 0 | 0 | 0 | 0 | 0 | −45.83 | 1.04 | 0.92 |
| O43390-4 | HNRNPR | 4\|0\|2 | 6 | 2 | 3\|3\|3 | 0 | 0 | 0 | 0 | 0 | −14.75 | 0.67 | 0.87 |
| P63104 | YWHAZ | 10\|5\|6 | 21 | 7 | 9\|7\|8 | 0 | 0 | 0 | 0 | 0 | −24.09 | 0.88 | 0.91 |
| Q13309 | SKP2 | 3\|0\|2 | 5 | 1.67 | 3\|4\|4 | 0 | 0 | 0 | 0 | 0 | −17.71 | 0.45 | 0.92 |
| Q9ULX3 | NOB1 | 6\|6\|5 | 17 | 5.67 | 4\|5\|4 | 0 | 0 | 0 | 0 | 0 | −10.78 | 1.31 | 0.88 |
| P29692-3 | EEFID | 11\|10\|13 | 34 | 11.33 | 7\|8\|8 | 0 | 0 | 0 | 0 | 0 | −15.32 | 1.48 | 0.89 |
| Q14677 | CLINT1 | 2\|4\|4 | 10 | 3.33 | 4\|5\|5 | 0 | 0 | 0 | 0 | 0 | −16.88 | 0.71 | 0.92 |
| Q96RQ3 | MCCC1 | 10\|109\|10 | 326 | 108.67 | 07\|13\|10 | 0 | 0 | 0 | 0 | 0 | −230.55 | 1 | 0.92 |
| P40692 | MLH1 | 2\|0\|4 | 6 | 2 | 7\|3\|4 | 0 | 0 | 0 | 0 | 0 | −22.08 | 0.43 | 0.92 |
| Q12906 | ILF3 | 20\|21\|28 | 69 | 23 | 29\|37\|29 | 0 | 0 | 0 | 0 | 0 | −87.49 | 0.73 | 0.92 |
| Q99623 | PHB2 | 7\|6\|5 | 18 | 6 | 8\|6\|9 | 0 | 0 | 0 | 0 | 0 | −22.85 | 0.78 | 0.92 |
| Q9H0B6-2 | KLC2 | 5\|3\|3 | 11 | 3.67 | 2\|3\|3 | 0 | 0.01 | 0 | 0.01 | 0 | −7.81 | 1.38 | 0.83 |
| Q7Z2W4 | ZC3HAV1 | 21\|14\|18 | 53 | 17.67 | 16\|13\|21 | 0 | 0 | 0 | 0 | 0 | −41.33 | 1.06 | 0.92 |
| P04406 | GAPDH | 38\|34\|32 | 104 | 34.67 | 37\|36\|30 | 0 | 0 | 0 | 0 | 0 | −77.39 | 1.01 | 0.92 |
| P61764 | STXBP1 | 2\|2\|3 | 7 | 2.33 | 4\|2\|2 | 0 | 0 | 0 | 0 | 0 | −9.29 | 0.88 | 0.87 |
| P11177-2 | PDHB | 11\|9\|11 | 31 | 10.33 | 7\|12\|8 | 0 | 0 | 0 | 0 | 0 | −21.43 | 1.15 | 0.91 |
| P55039 | DRG2 | 6\|6\|7 | 19 | 6.33 | 4\|5\|5 | 0 | 0 | 0 | 0 | 0 | −10.52 | 1.36 | 0.88 |
| P55036 | PSMD4 | 2\|3\|2 | 7 | 2.33 | 0\|2\|4 | 0 | 0.01 | 0 | 0.01 | 0 | −6.67 | 1.17 | 0.83 |
| O9NSD9 | FARSB | 10\|11\|11 | 32 | 10.67 | 11\|11\|9 | 0 | 0 | 0 | 0 | 0 | −24.68 | 1.03 | 0.91 |
| PS2298-3 | NCBP2 | 0\|0\|3 | 3 | 1 | 2\|0\|3 | 0 | 0.01 | 0 | 0.01 | 0 | −9.13 | 0.6 | 0.81 |
| Q13490-2 | BIRC2 | 9\|3\|8 | 20 | 6.67 | 7\|7\|8 | 0 | 0 | 0 | 0 | 0 | −25.42 | 0.91 | 0.9 |
| Q9NZI8 | GF2BP1 | 9\|6\|7 | 22 | 7.33 | 11\|8\|8 | 0 | 0 | 0 | 0 | 0 | −26.11 | 0.81 | 0.92 |
| P62070 | RRAS2 | 2\|3\|3 | 8 | 2.67 | 2\|2\|3 | 0 | 0 | 0 | 0 | 0 | −8.1 | 1.14 | 0.85 |
| Q92769-3 | HDAC2 | 7\|6\|11 | 24 | 8 | 5\|10\|6 | 0 | 0 | 0 | 0 | 0 | −18.72 | 1.14 | 0.89 |
| P62277 | RPS13 | 3\|4\|4 | 11 | 3.67 | 5\|5\|3 | 0 | 0 | 0 | 0 | 0 | −13.76 | 0.85 | 0.92 |
| Q9UJV9 | DDX41 | 4\|2\|3 | 9 | 3 | 6\|4\|5 | 0 | 0 | 0 | 0 | 0 | −18.16 | 0.6 | 0.92 |
| Q96EP5-2 | DAZAP1 | 3\|0\|2 | 5 | 1.67 | 2\|2\|3 | 0 | 0 | 0 | 0 | 0 | −11.93 | 0.71 | 0.86 |
| Q53GS9 | USP39 | 5\|2\|4 | 11 | 3.67 | 2\|5\|4 | 0 | 0 | 0 | 0 | 0 | −12.99 | 1 | 0.88 |
| P05023 | ATPIA1 | 7\|8\|6 | 21 | 7 | 6\|7\|7 | 0 | 0 | 0 | 0 | 0 | −17.57 | 1.05 | 0.9 |
| Q5T6F2 | UBAP2 | 3\|6\|4 | 13 | 4.33 | 5\|4\|3 | 0 | 0 | 0 | 0 | 0 | −12.5 | 1.08 | 0.87 |
| Q07065 | CKAP4 | 47\|44\|48 | 139 | 46.33 | 48\|49\|47 | 0 | 0 | 0 | 0 | 0 | −108.22 | 0.97 | 0.92 |
| O75694-2 | NUP155 | 2\|2\|3 | 7 | 2.33 | 4\|3\|4 | 0 | 0 | 0 | 0 | 0 | −12.99 | 0.64 | 0.92 |
| P50990 | CCT8 | 33\|29\|30 | 92 | 30.67 | 42\|50\|44 | 0 | 0 | 0 | 0 | 0 | −123.46 | 0.68 | 0.92 |
| Q9UBF8-2 | PI4KB | 2\|0\|2 | 4 | 1.33 | 2\|2\|2 | 0 | 0 | 0 | 0 | 0 | −10.44 | 0.67 | 0.92 |
| Q16401-2 | PSMD5 | 2\|0\|2 | 4 | 1.33 | 3\|0\|3 | 0 | 0 | 0 | 0 | 0 | −10.34 | 0.67 | 0.92 |
| P31040 | SDHA | 13\|13\|16 | 42 | 14 | 20\|17\|19 | 0 | 0 | 0 | 0 | 0 | −50.44 | 0.75 | 0.92 |
| P51153 | RAB13 | 4\|5\|5 | 14 | 4.67 | 6\|5\|5 | 0 | 0 | 0 | 0 | 0 | −15.82 | 0.88 | 0.92 |
| P51151 | RAB9A | 4\|2\|5 | 11 | 3.67 | 3\|3\|3 | 0 | 0 | 0 | 0 | 0 | −10.42 | 1.22 | 0.85 |
| Q96124 | FUBP3 | 5\|5\|8 | 18 | 6 | 5\|6\|5 | 0 | 0 | 0 | 0 | 0 | −14.32 | 1.12 | 0.89 |
| O00303 | EIF3F | 11\|10\|10 | 31 | 10.33 | 11\|12\|11 | 0 | 0 | 0 | 0 | 0 | −28.23 | 0.91 | 0.92 |
| P02786 | TFRC | 4\|3\|3 | 10 | 3.33 | 0\|3\|4 | 0 | 0.01 | 0 | 0.01 | 0 | −6.41 | 1.43 | 0.82 |
| O14579 | COPE | 7\|9\|6 | 22 | 7.33 | 8\|10\|7 | 0 | 0 | 0 | 0 | 0 | −23.63 | 0.88 | 0.91 |
| Q9UL25 | RAB21 | 4\|4\|6 | 14 | 4.67 | 5\|7\|4 | 0 | 0 | 0 | 0 | 0 | −15.82 | 0.88 | 0.9 |
| P0DMV9 | HSPA1B | 53\|43\|41 | 137 | 45.67 | 46\|41\|40 | 0 | 0 | 0 | 0 | 0 | −92.5 | 1.08 | 0.92 |
| P18621 | RPL17 | 6\|7\|7 | 20 | 6.67 | 5\|5\|8 | 0 | 0 | 0 | 0 | 0 | −15.21 | 1.11 | 0.9 |
| P62854 | RPS26 | 4\|4\|5 | 13 | 4.33 | 5\|5\|7 | 0 | 0 | 0 | 0 | 0 | −17.06 | 0.76 | 0.92 |
| P52597 | HNRNPF | 26\|27\|27 | 80 | 26.67 | 28\|31\|32 | 0 | 0 | 0 | 0 | 0 | −72.19 | 0.88 | 0.92 |
| P17655 | CAPN2 | 5\|4\|5 | 14 | 4.67 | 7\|10\|6 | 0 | 0 | 0 | 0 | 0 | −24.7 | 0.61 | 0.92 |
| P09211 | GSTP1 | 4\|4\|7 | 15 | 5 | 5\|5\|4 | 0 | 0 | 0 | 0 | 0 | −13.43 | 1.07 | 0.88 |
| Q13310-2 | PABPC4 | 2\|0\|3 | 5 | 1.67 | 2\|3\|4 | 0 | 0 | 0 | 0 | 0 | −14.75 | 0.56 | 0.92 |
| Q9BZX2 | UCK2 | 2\|2\|2 | 6 | 2 | 5\|4\|3 | 0 | 0 | 0 | 0 | 0 | −14.21 | 0.5 | 0.92 |
| A1X283 | SH3PXD2B | 8\|3\|6 | 17 | 5.67 | 7\|6\|6 | 0 | 0 | 0 | 0 | 0 | −21.43 | 0.89 | 0.9 |
| Q9H974-2 | QTRT2 | 3\|4\|3 | 10 | 3.33 | 2\|4\|4 | 0 | 0 | 0 | 0 | 0 | −10.17 | 1 | 0.88 |
| Q15020 | SART3 | 6\|7\|8 | 21 | 7 | 13\|4\|6 | 0 | 0 | 0 | 0 | 0 | −20.78 | 0.91 | 0.91 |
| Q9Y277 | VDAC3 | 6\|7\|6 | 19 | 6.33 | 8\|8\|8 | 0 | 0 | 0 | 0 | 0 | −22.37 | 0.79 | 0.92 |
| Q14914-2 | PTGR1 | 4\|5\|9 | 18 | 6 | 2\|3\|7 | 0 | 0.01 | 0 | 0.01 | 0 | −10.89 | 1.5 | 0.82 |
| P63092-3 | GNAS | 4\|3\|3 | 10 | 3.33 | 3\|4\|4 | 0 | 0 | 0 | 0 | 0 | −11.36 | 0.91 | 0.89 |
| P30453 | HLA-A | 3\|4\|4 | 11 | 3.67 | 5\|3\|0 | 0 | 0 | 0 | 0 | 0 | −7.46 | 1.38 | 0.84 |
| P35610 | SOAT1 | 3\|2\|2 | 7 | 2.33 | 2\|2\|3 | 0 | 0 | 0 | 0 | 0 | −8.1 | 1 | 0.86 |
| P00403 | MT-CO2 | 2\|2\|3 | 7 | 2.33 | 4\|4\|2 | 0 | 0 | 0 | 0 | 0 | −11.71 | 0.7 | 0.92 |
| P17812 | CTPS1 | 12\|10\|11 | 33 | 11 | 11\|9\|10 | 0 | 0 | 0 | 0 | 0 | −23.5 | 1.1 | 0.91 |
| 45880-2 | VDAC2 | 2\|4\|5 | 11 | 3.67 | 4\|3\|5 | 0 | 0 | 0 | 0 | 0 | −14.21 | 0.92 | 0.88 |
| Q64102-2 | WASHC2A | 2\|3\|0 | 5 | 1.67 | 2\|3\|5 | 0 | 0 | 0 | 0 | 0 | −16.24 | 0.5 | 0.92 |
| P08670 | VIM | 68\|70\|69 | 207 | 69 | 74\|76 \|70 | 0 | 0 | 0 | 0 | 0 | −162.73 | 0.94 | 0.92 |
| P32969 | RPL9P8; | 12\|8\|9 | 29 | 9.67 | 9 \|13 \|21 | 0 | 0 | 0 | 0 | 0 | −42.62 | 0.67 | 0.92 |
| | RPL9; | | | | | | | | | | | | |
| | RPL91 | | | | | | | | | | | | |
| Q13177 | PAK2 | 5\|4\|6 | 15 | 5 | 6\|7\|7 | 0 | 0 | 0 | 0 | 0 | −20.83 | 0.75 | 0.92 |
| O43747 | AP1G1 | 0\|2\|0 | 2 | 0.67 | 2\|0\|2 | 0 | 0.01 | 0 | 0.01 | 0 | −7.8 | 0.5 | 0.82 |
| P42224 | STATI | 6\|8\|8 | 22 | 7.33 | 14\|8\|9 | 0 | 0 | 0 | 0 | 0 | −31.24 | 0.71 | 0.92 |
| P22234 | PAICS | 13\|13\|17 | 43 | 14.33 | 15\|14\|12 | 0 | 0 | 0 | 0 | 0 | −32.08 | 1.05 | 0.91 |
| P04792 | HSPB1 | 17\|19\|18 | 54 | 18 | 18\|20\|15 | 0 | 0 | 0 | 0 | 0 | −40.37 | 1.02 | 0.92 |
| O43684-2 | BUB3 | 13\|10\|11 | 34 | 11.33 | 16\|13\|11 | 0 | 0 | 0 | 0 | 0 | −35.55 | 0.85 | 0.92 |
| Q09666 | AHNAK | 25\|137\|13 | 393 | 131 | 09\|239\|21 | 0 | 0 | 0 | 0 | 0 | −621.06 | 0.59 | 0.92 |
| Q15424-2 | ISAFB | 2\|4\|4 | 10 | 3.33 | 9\|7\|9 | 0 | 0 | 0 | 0 | 0 | −31.91 | 0.4 | 0.92 |
| O15091-2 | KIAA0391 | 2\|0\|0 | 2 | 0.67 | 0\|2\|2 | 0 | 0.01 | 0 | 0.01 | 0 | −7.8 | 0.5 | 0.82 |
| P55209-3 | NAP1L1 | 3\|3\|3 | 9 | 3 | 0\|3\|3 | 0 | 0 | 0 | 0 | 0 | −5.36 | 1.5 | 0.81 |
| P14625 | HSP9081 | 7\|6\|6 | 19 | 6.33 | 7\|6\|6 | 0 | 0 | 0 | 0 | 0 | −16.39 | 3 | 0.9 |
| P62140 | PPP1CB | 11\|9\|9 | 29 | 9.67 | 7\|9\|9 | 0 | 0 | 0 | 0 | 0 | −19.08 | 1.16 | 0.91 |
| P13929-3 | ENO3 | 3\|3\|6 | 12 | 4 | 3\|6\|6 | 0 | 0 | 0 | 0 | 0 | −16.23 | 0.8 | 0.89 |
| Q6UWE0 | LRSAM1 | 5\|3\|3 | 11 | 3.67 | 9\|11\|5 | 0 | 0 | 0 | 0 | 0 | −29.52 | 0.44 | 0.92 |
| Q969U7-2 | PSMG2 | 4\|4\|4 | 12 | 4 | 4\|4\|3 | 0 | 0 | 0 | 0 | 0 | −9.88 | 1.09 | 0.88 |
| Q9P0J7 | KCMF1 | 414\|4 | 12 | 4 | 0\|4\|5 | 0 | 0 | 0 | 0 | 0 | −7.2 | 1.33 | 0.85 |
| Q96584-4 | SRPK1 | 4\|3\|5 | 12 | 4 | 4\|3\|5 | 0 | 0 | 0 | 0 | 0 | −12.5 | 1 | 0.88 |
| Q86TX2 | ACOT1 | 0\|0\|3 | 3 | 1 | 4\|3\|2 | 0 | 0 | 0 | 0 | 0 | −14.75 | 0.33 | 0.92 |
| O95573 | ACSL3 | 15\|11\|14 | 40 | 13.33 | 13\|11\|9 | 0 | 0 | 0 | 0 | 0 | −25.58 | 1.21 | 0.91 |
| P09651-3 | HNRNPA1 | 10\|7\|5 | 22 | 7.33 | 6\|8\|12 | 0 | 0 | 0 | 0 | 0 | −26.74 | 0.85 | 0.91 |
| O14979 | HNRNPDL | 5\|5\|4 | 14 | 4.67 | 6\|5\|8 | 0 | 0 | 0 | 0 | 0 | −19.55 | 0.74 | 0.92 |
| Q8WUM0 | NUP133 | 4\|5\|3 | 12 | 4 | 6\|7\|6 | 0 | 0 | 0 | 0 | 0 | −21.43 | 0.63 | 0.92 |
| P07237 | P4HB | 3\|2\|0 | 5 | 1.67 | 2\|4\|3 | 0 | 0 | 0 | 0 | 0 | −14.75 | 0.56 | 0.92 |
| P47756-2 | CAPZB | 9\|9\|10 | 28 | 9.33 | 13\|7\|11 | 0 | 0 | 0 | 0 | 0 | −26.16 | 0.9 | 0.92 |
| Q9H9A6 | LRRC40 | 12\|11\|9 | 32 | 10.67 | 14\|7\|11 | 0 | 0 | 0 | 0 | 0 | −27.4 | 1 | 0.91 |
| P11387 | TOP1 | 4\|5\|5 | 14 | 4.67 | 7\|4\|4 | 0 | 0 | 0 | 0 | 0 | −14.57 | 0.93 | 0.92 |
| Q9UG63 | ABCF2 | 10\|17\|15 | 42 | 14 | 10\|13\|11 | 0 | 0 | 0 | 0 | 0 | −28.23 | 1.24 | 0.91 |
| Q14683 | SMC1A | 11\|14\|9 | 34 | 11.33 | 8\|10\|6 | 0 | 0 | 0 | 0 | 0 | −17.91 | 1.42 | 0.89 |
| P04899 | GNA12 | 4\|4\|4 | 12 | 4 | 4\|4\|3 | 0 | 0 | 0 | 0 | 0 | −9.88 | 1.09 | 0.88 |
| P06493 | CDK1 | 12\|11\|9 | 32 | 10.67 | 9\|11\|6 | 0 | 0 | 0 | 0 | 0 | −20.25 | 1.23 | 0.91 |
| O43491-4 | EPB41L2 | 7\|3\|0 | 10 | 3.33 | 4\|3\|4 | 0 | 0 | 0 | 0 | 0 | −17.71 | 0.91 | 0.85 |
| P62847-2 | RPS24 | 5\|5\|5 | 15 | 5 | 7\|4\|6 | 0 | 0 | 0 | 0 | 0 | −15.5 | 0.88 | 0.92 |
| P05787 | KRT8 | 46\|44\|48 | 138 | 46 | 47\|56\|53 | 0 | 0 | 0 | 0 | 0 | −122.73 | 0.88 | 0.92 |
| P05783 | KRT18 | 38\|32\|29 | 99 | 33 | 34\|41\|34 | 0 | 0 | 0 | 0 | 0 | −89.39 | 0.91 | 0.92 |
| P11166 | SLC2A1 | 3\|3\|4 | 10 | 3.33 | 4\|2\|2 | 0 | 0 | 0 | 0 | 0 | −7.81 | 1.25 | 0.85 |
| O9Y520-4 | PRRC2C | 7\|6\|12 | 25 | 8.33 | 11\|5\|12 | 0 | 0 | 0 | 0 | 0 | −27.29 | 0.89 | 0.91 |
| P47914 | RPL29 | 5\|5\|5 | 15 | 5 | 4\|4\|4 | 0 | 0 | 0 | 0 | 0 | −9.61 | 1.25 | 0.88 |
| Q8WX19 | GATAD2B | 4\|3\|7 | 14 | 4.67 | 5\|4\|5 | 0 | 0 | 0 | 0 | 0 | −15.01 | 1 | 0.88 |
| Q9NVM9 | INTS13 | 0\|3\|3 | 6 | 2 | 4\|2\|2 | 0 | 0 | 0 | 0 | 0 | −13.39 | 0.75 | 0.86 |
| O43318-2 | MAP3K7 | 3\|0\|2 | 5 | 1.67 | 4\|4\|4 | 0 | 0 | 0 | 0 | 0 | −13.22 | 0.63 | 0.87 |
| P61513 | RPL37A | 2\|2\|3 | 7 | 2.33 | 3\|2\|3 | 0 | 0 | 0 | 0 | 0 | −9.29 | 0.88 | 0.87 |
| Q9UBQ0 | VPS29 | 0\|0\|2 | 2 | 0.67 | 2\|2\|2 | 0 | 0 | 0 | 0 | 0 | −10.44 | 0.33 | 0.92 |
| Q9NR45 | NANS | 2\|4\|4 | 10 | 3.33 | 3\|2\|3 | 0 | 0 | 0 | 0 | 0 | −9.29 | 1.25 | 0.85 |
| Q9NQT5-2 | EXOSC3 | 2\|2\|2 | 6 | 2 | 3\|2\|3 | 0 | 0 | 0 | 0 | 0 | −9.29 | 0.75 | 0.92 |
| Q13546 | RIPK1 | 3\|3\|3 | 9 | 3 | 0\|4\|4 | 0 | 0 | 0 | 0 | 0 | −7.55 | 1.12 | 0.86 |
| P62081 | RPS7 | 22\|25\|29 | 76 | 25.33 | 18\|21\|25 | 0 | 0 | 0 | 0 | 0 | −46.04 | 1.19 | 0.92 |
| P62241 | RPS8 | 14\|15\|12 | 41 | 13.67 | 16\|15\|15 | 0 | 0 | 0 | 0 | 0 | −39.65 | 0.89 | 0.92 |
| P62244 | RPS15A | 11\|12\|11 | 34 | 11.33 | 11\|11\|13 | 0 | 0 | 0 | 0 | 0 | −27.93 | 0.97 | 0.91 |
| P50454 | SERPINH1 | 14\|15\|18 | 47 | 15.67 | 18\|16\|16 | 0 | 0 | 0 | 0 | 0 | −41.33 | 0.94 | 0.92 |
| P62249 | RPS16 | 14\|14\|14 | 42 | 14 | 12\|12\|12 | 0 | 0 | 0 | 0 | 0 | −24.77 | 1.17 | 0.91 |
| P12236 | SLC25A6 | 13\|11\|12 | 36 | 12 | 12\|14\|12 | 0 | 0 | 0 | 0 | 0 | −31.5 | 0.95 | 0.92 |
| P12235 | SLC25A4 | 8\|6\|8 | 22 | 7.33 | 6\|8\|6 | 0 | 0 | 0 | 0 | 0 | −17.57 | 1.1 | 0.9 |
| O95487-2 | SEC24B | 2\|2\|0 | 4 | 1.33 | 3\|0\|2 | 0 | 0 | 0 | 0 | 0 | −9.13 | 0.8 | 0.83 |
| Q9NP64-2 | ZCCHC17 | 2\|3\|4 | 9 | 3 | 6\|8\|7 | 0 | 0 | 0 | 0 | 0 | −26.33 | 0.43 | 0.92 |
| O60506-4 | SYNCRIP | 7\|5\|5 | 17 | 5.67 | 7\|8\|10 | 0 | 0 | 0 | 0 | 0 | −25.39 | 0.68 | 0.92 |
| Q09028-4 | RBBP4 | 5\|5\|5 | 15 | 5 | 5\|6\|4 | 0 | 0 | 0 | 0 | 0 | −13.14 | 1 | 0.92 |
| O94986-3 | CEP152 | 2\|3\|3 | 8 | 2.67 | 2\|2\|4 | 0 | 0 | 0 | 0 | 0 | −9.29 | 1 | 0.86 |
| P34931 | HSPA1L | 21\|15\|16 | 52 | 17.33 | 13\|15\|15 | 0 | 0 | 0 | 0 | 0 | −31.53 | 1.21 | 0.91 |
| P34932 | HSPA4 | 8\|5\|12 | 25 | 8.33 | 5\|5\|8 | 0 | 0 | 0 | 0 | 0 | −16.65 | 1.39 | 0.86 |
| P51149 | RAB7A | 13\|10\|12 | 35 | 11.67 | 13\|11\|14 | 0 | 0 | 0 | 0 | 0 | −33.08 | 0.92 | 0.92 |
| P51148 | RAB5C | 6\|9\|9 | 24 | 8 | 9\|7\|9 | 0 | 0 | 0 | 0 | 0 | −23.63 | 0.96 | 0.91 |
| P05166 | PCCB | 19\|18\|16 | 53 | 17.67 | 24\|22\|20 | 0 | 0 | 0 | 0 | 0 | −57.78 | 0.8 | 0.92 |
| P05165 | PCCA | 49\|149\|15 | 448 | 149.33 | 75\|170\|18 | 0 | 0 | 0 | 0 | 0 | −410.1 | 0.85 | 0.92 |
| P08559-3 | PDHA1 | 10\|11\|12 | 33 | 11 | 11\|11\|11 | 0 | 0 | 0 | 0 | 0 | −27.02 | 1 | 0.91 |
| Q6RFH5 | WDR74 | 6\|7\|10 | 23 | 7.67 | 3\|8\|4 | 0 | 0 | 0 | 0 | 0 | −11.62 | 1.53 | 0.85 |
| P55265 | ADAR | 18\|16\|19 | 53 | 17.67 | 18\|23\|17 | 0 | 0 | 0 | 0 | 0 | −47.89 | 0.91 | 0.92 |
| P10412 | HIST1H1E | 9\|6\|4 | 19 | 6.33 | 8\|6\|3 | 0 | 0 | 0 | 0 | 0 | −17.1 | 1.12 | 0.89 |
| Q9UHD8-7 | 9-Sep | 11\|12\|12 | 35 | 11.67 | 10\|11\|15 | 0 | 0 | 0 | 0 | 0 | −29.1 | 0.97 | 0.92 |
| P30050 | RPL12 | 10\|4\|7 | 21 | 7 | 6\|5\|9 | 0 | 0 | 0 | 0 | 0 | −20.83 | 1.05 | 0.9 |
| P35998 | PSMC2 | 16\|17\|15 | 48 | 16 | 14\|20\|17 | 0 | 0 | 0 | 0 | 0 | −40.97 | 0.94 | 0.92 |
| P55795 | HNRNPH2 | 25\|21\|22 | 68 | 22.67 | 25\|25\|33 | 0 | 0 | 0 | 0 | 0 | −70.5 | 0.82 | 0.92 |
| O905140 | MFN2 | 2\|0\|3 | 5 | 1.67 | 0\|3\|3 | 0 | 0 | 0 | 0 | 0 | −10.34 | 0.83 | 0.84 |
| PS5084-2 | HADHB | 5\|3\|4 | 12 | 4 | 3\|3\|4 | 0 | 0 | 0 | 0 | 0 | −10.17 | 1.2 | 0.86 |
| P08729 | KRT7 | 44\|39\|43 | 126 | 42 | 45\|43\|41 | 0 | 0 | 0 | 0 | 0 | −97.87 | 0.98 | 0.92 |
| Q9NNWS | WDR6 | 0\|3\|0 | 3 | 3 | 3\|2\|2 | 0 | 0 | 0 | 0 | 0 | −11.93 | 0.43 | 0.86 |
| P60893 | PRPS1 | 7\|6\|9 | 22 | 7.33 | 6\|10\|8 | 0 | 0 | 0 | 0 | 0 | −22.37 | 0.92 | 0.91 |
| Q9HAU0-5 | PLEKHAS | 2\|2\|0 | 4 | 1.33 | 2\|4\|3 | 0 | 0 | 0 | 0 | 0 | −14.75 | 0.44 | 0.92 |
| Q13409-3 | DYNC112 | 11\|6\|9 | 26 | 8.67 | 5\|6\|8 | 0 | 0 | 0 | 0 | 0 | −16.39 | 1.37 | 0.88 |
| Q86UK7-2 | ZNF598 | 9\|9\|8 | 26 | 8.67 | 9\|9\|6 | 0 | 0 | 0 | 0 | 0 | −19.36 | 1.08 | 0.91 |
| Q8WWI1-3 | LMO7 | 15\|15\|11 | 41 | 13.67 | 10\|12\|15 | 0 | 0 | 0 | 0 | 0 | −30.3 | 1.11 | 0.91 |
| O15143 | ARPC1B | 9\|6\|9 | 24 | 8 | 11\|9\|11 | 0 | 0 | 0 | 0 | 0 | −31.24 | 0.77 | 0.92 |
| O15144 | ARPC2 | 3\|3\|3 | 9 | 3 | 3\|5\|4 | 0 | 0 | 0 | 0 | 0 | −12.5 | 0.75 | 0.92 |
| O15145 | ARPC3 | 3\|4\|3 | 10 | 3.33 | 4\|5\|3 | 0 | 0 | 0 | 0 | 0 | −12.5 | 0.83 | 0.92 |
| Q96KP4 | CNDP2 | 7\|8\|4 | 19 | 6.33 | 6\|7\|6 | 0 | 0 | 0 | 0 | 0 | −19.55 | 1 | 0.9 |
| Q9Y266 | NUDC | 2\|2\|2 | 6 | 2 | 3\|0\|3 | 0 | 0 | 0 | 0 | 0 | −6.82 | 3 | 0.92 |
| Q9Y265 | RUVBL1 | 4\|5\|5 | 14 | 4.67 | 6\|4\|4 | 0 | 0 | 0 | 0 | 0 | −13.43 | 1 | 0.89 |
| Q9Y263 | PLAA | 11\|12\|12 | 35 | 11.67 | 12\|10\|11 | 0 | 0 | 0 | 0 | 0 | −25.58 | 1.06 | 0.91 |
| Q08211 | DHX9 | 35\|35\|39 | 109 | 36.33 | 43\|43\|44 | 0 | 0 | 0 | 0 | 0 | −105.42 | 0.84 | 0.92 |
| P11310 | ACADM | 3\|2\|2 | 7 | 2.33 | 4\|3\|0 | 0 | 0 | 0 | 0 | 0 | −7.93 | 1 | 0.85 |
| Q5TFE4 | NTSDC1 | 4\|4\|5 | 13 | 4.33 | 6\|7\|9 | 0 | 0 | 0 | 0 | 0 | −23.39 | 0.59 | 0.92 |
| QSTAT6 | NPLOC4 | 7\|4\|7 | 18 | 6 | 4\|9\|7 | 0 | 0 | 0 | 0 | 0 | −20.83 | 0.9 | 0.9 |
| P21333-2 | FLNA | 34\|35\|30 | 99 | 33 | 55\|48\|50 | 0 | 0 | 0 | 0 | 0 | −143.56 | 0.65 | 0.92 |
| P63010-3 | AP281 | 8\|6\|7 | 21 | 7 | 5\|4\|4 | 0 | 0 | 0 | 0 | 0 | −9.36 | 1.62 | 0.85 |
| P46777 | RPLS | 35\|36\|28 | 99 | 33 | 33\|35\|29 | 0 | 0 | 0 | 0 | 0 | −76.32 | 1.02 | 0.92 |
| O43823 | AKAP8 | 6\|6\|7 | 19 | 6.33 | 8\|6\|5 | 0 | 0 | 0 | 0 | 0 | −16.39 | 1 | 0.9 |
| O95816 | BAG2 | 3\|3\|3 | 9 | 3 | 2\|2\|2 | 0 | 0 | 0 | 0 | 0 | −5.46 | 1.5 | 0.81 |
| Q01085-2 | ITIAL1 | 6\|7\|9 | 22 | 7.33 | 7\|7\|6 | 0 | 0 | 0 | 0 | 0 | −17.57 | 1.1 | 0.9 |
| Q99798 | ACO2 | 6\|4\|5 | 15 | 5 | 10\|7\|9 | 0 | 0 | 0 | 0 | 0 | −28.65 | 0.58 | 0.92 |
| Q13148 | TARDBP | 8\|10\|9 | 27 | 9 | 9\|11\|10 | 0 | 0 | 0 | 0 | 0 | −26.49 | 0.9 | 0.92 |
| Q13144 | EIF2BS | 3\|2\|2 | 7 | 2.33 | 2\|2\|3 | 0 | 0 | 0 | 0 | 0 | −8.1 | 1 | 0.86 |
| Q00325-2 | SLC25A3 | 14\|11\|14 | 39 | 13 | 14\|13\|13 | 0 | 0 | 0 | 0 | 0 | −33.92 | 0.97 | 0.92 |
| Q92785 | DPF2 | 2\|0\|0 | 2 | 0.67 | 0\|2\|2 | 0 | 0.01 | 0 | 0.01 | 0 | −7.8 | 0.5 | 0.82 |
| P46087 | NOP2 | 4\|3\|6 | 13 | 4.33 | 6\|4\|3 | 0 | 0 | 0 | 0 | 0 | −13.76 | 1 | 0.88 |
| P54578-2 | USP14 | 6\|7\|6 | 19 | 6.33 | 6\|4\|5 | 0 | 0 | 0 | 0 | 0 | −11.69 | 1.27 | 0.89 |
| Q01469 | FABPS | 3\|2\|5 | 10 | 3.33 | 3\|3\|3 | 0 | 0 | 0 | 0 | 0 | −10.42 | 1.11 | 0.85 |
| P35579 | MYH9 | 19\|14\|17 | 50 | 16.67 | 23\|24\|21 | 0 | 0 | 0 | 0 | 0 | −63.93 | 0.74 | 0.92 |
| O14929-2 | HAT1 | 0\|2\|0 | 2 | 0.67 | 2\|2\|2 | 0 | 0 | 0 | 0 | 0 | −10.44 | 0.33 | 0.92 |
| P62888 | RPL30 | 3\|6\|6 | 15 | 5 | 8\|7\|7 | 0 | 0 | 0 | 0 | 0 | −25.42 | 0.68 | 0.92 |
| Q96P11-2 | NSUN5 | 0\|0\|2 | 2 | 0.67 | 2\|2\|4 | 0 | 0 | 0 | 0 | 0 | −13.39 | 0.25 | 0.92 |
| O43175 | PHGDH | 0\|0\|2 | 2 | 0.67 | 3\|2\|2 | 0 | 0 | 0 | 0 | 0 | −11.93 | 0.29 | 0.92 |
| Q9NYB9-2 | ABI2 | 5\|4\|5 | 14 | 4.67 | 4\|6\|3 | 0 | 0 | 0 | 0 | 0 | −12.24 | 1.08 | 0.89 |
| P26599 | PTBP1 | 9\|7\|11 | 27 | 9 | 9\|11\|14 | 0 | 0 | 0 | 0 | 0 | −33.24 | 0.79 | 0.92 |
| O43396 | TXNL1 | 3\|3\|3 | 9 | 3 | 3\|5\|0 | 0 | 0 | 0 | 0 | 0 | −7.46 | 1.12 | 0.85 |
| O43395 | PRPF3 | 0\|2\|0 | 2 | 0.67 | 6\|4\|4 | 0 | 0 | 0 | 0 | 0 | −22.08 | 0.14 | 0.92 |
| Q13557-12 | CAMK2D | 2\|2\|2 | 6 | 2 | 2\|2\|2 | 0 | 0 | 0 | 0 | 0 | −6.91 | 1 | 0.92 |
| P12956 | XRCC6 | 26\|23\|23 | 72 | 24 | 30\|27\|26 | 0 | 0 | 0 | 0 | 0 | −67.21 | 0.87 | 0.92 |
| P23526 | AHCY | 35\|34\|40 | 109 | 36.33 | 32\|36\|36 | 0 | 0 | 0 | 0 | 0 | −75.63 | 1.05 | 0.92 |
| P23527 | HIST1H2B0 | 4\|5\|5 | 14 | 4.67 | 7\|9\|6 | 0 | 0 | 0 | 0 | 0 | −23.39 | 0.64 | 0.92 |
| P09874 | PARP1 | 36\|34\|39 | 109 | 36.33 | 44\|38\|42 | 0 | 0 | 0 | 0 | 0 | −99.67 | 0.88 | 0.92 |
| P23528 | CFL1 | 9\|10\|9 | 28 | 9.33 | 12\|12\|18 | 0 | 0 | 0 | 0 | 0 | −39.8 | 0.67 | 0.92 |
| Q96PK6-5 | RBM14 | 6\|5\|5 | 16 | 5.33 | 7\|7\|6 | 0 | 0 | 0 | 0 | 0 | −19.11 | 0.8 | 0.92 |
| P19388 | POLR2E | 2\|2\|2 | 6 | 2 | 0\|3\|3 | 0 | 0 | 0 | 0 | 0 | −6.82 | 1 | 0.92 |
| O14828-2 | SCAMP3 | 6\|4\|6 | 16 | 5.33 | 8\|6\|9 | 0 | 0 | 0 | 0 | 0 | −24.7 | 0.7 | 0.92 |
| Q14697 | GANAB | 18\|19\|18 | 55 | 18.33 | 18\|19\|16 | 0 | 0 | 0 | 0 | 0 | −38.92 | 1.04 | 0.92 |
| P19387 | POLR2C | 5\|4\|5 | 14 | 4.67 | 4\|3\|3 | 0 | 0 | 0 | 0 | 0 | −8.71 | 1.4 | 0.86 |
| Q93009-3 | USP7 | 10\|8\|7 | 25 | 8.33 | 8\|9\|10 | 0 | 0 | 0 | 0 | 0 | −24.43 | 0.93 | 0.91 |
| P36507 | MAP2K2 | 3\|3\|3 | 9 | 3 | 3\|3\|2 | 0 | 0 | 0 | 0 | 0 | −7.81 | 1.12 | 0.86 |
| Q15654 | TRIP6 | 8\|10\|9 | 27 | 9 | 9\|10\|10 | 0 | 0 | 0 | 0 | 0 | −25.3 | 0.93 | 0.91 |
| P36873 | PPP1CC | 9\|7\|7 | 23 | 7.67 | 6\|8\|10 | 0 | 0 | 0 | 0 | 0 | −20.8 | 0.96 | 0.91 |
| P36873 | PGM1 | 3\|2\|4 | 9 | 3 | 4\|2\|2 | 0 | 0 | 0 | 0 | 0 | −9.29 | 1.12 | 0.85 |
| Q9UPT8 | ZC3H4 | 4\|4\|5 | 13 | 4.33 | 6\|4\|3 | 0 | 0 | 0 | 0 | 0 | −12.24 | 1 | 0.89 |
| Q3MHD2 | LSM12 | 0\|0\|2 | 2 | 0.67 | 3\|2\|3 | 0 | 0 | 0 | 0 | 0 | −13.39 | 0.25 | 0.92 |
| Q6DD87 | ZNF787 | 4\|2\|2 | 8 | 2.67 | 5\|0\|2 | 0 | 0.01 | 0 | 0.01 | 0 | −7.76 | 1.14 | 0.83 |
| Q6DD88 | ATL3 | 0\|2\|2 | 4 | 1.33 | 3\|0\|3 | 0 | 0 | 0 | 0 | 0 | −10.34 | 0.67 | 0.92 |
| Q9Y508-2 | GTF3C5 | 5\|5\|4 | 14 | 4.67 | 5\|5\|4 | 0 | 0 | 0 | 0 | 0 | −13.43 | 1 | 0.89 |
| Q9BRA2 | TXNDC17 | 2\|2\|4 | 8 | 2.67 | 2\|3\|2 | 0 | 0.01 | 0 | 0.01 | 0 | −8.1 | 1.14 | 0.84 |
| Q96176-9 | MMS19 | 0\|2\|0 | 2 | 0.67 | 0\|2\|2 | 0 | 0.01 | 0 | 0.01 | 0 | −7.8 | 0.5 | 0.82 |
| Q9BQ04 | RBM4B | 0\|2\|3 | 5 | 1.67 | 4\|2\|5 | 0 | 0 | 0 | 0 | 0 | −17.71 | 0.45 | 0.92 |
| Q5T6V5 | C9ORF64 | 0\|0\|2 | 2 | 0.67 | 3\|2\|0 | 0 | 0 | 0 | 0 | 0 | −9.13 | 0.4 | 0.84 |
| Q9UBU8-2 | MORF4L1 | 4\|4\|4 | 12 | 4 | 2\|3\|3 | 0 | 0 | 0 | 0 | 0 | −6.37 | 1.5 | 0.84 |
| P06241 | FYN | 5\|4\|6 | 15 | 5 | 5\|5\|4 | 0 | 0 | 0 | 0 | 0 | −13.43 | 1.07 | 0.89 |
| P22695 | UQCRC2 | 10\|12\|12 | 34 | 11.33 | 11\|10\|11 | 0 | 0 | 0 | 0 | 0 | −25.86 | 1.06 | 0.91 |
| P22694 | PRKACB | 4\|3\|2 | 9 | 3 | 2\|4\|3 | 0 | 0 | 0 | 0 | 0 | −10.42 | 1 | 0.87 |
| Q16555-2 | DPYSL2 | 8\|10\|5 | 23 | 7.67 | 5\|8\|6 | 0 | 0 | 0 | 0 | 0 | −17.88 | 1.21 | 0.89 |
| P11021 | HSPA5 | 13\|17\|14 | 44 | 14.67 | 14\|19\|16 | 0 | 0 | 0 | 0 | 0 | −41.71 | 0.9 | 0.92 |
| Q13620-1 | CUL4B | 4\|4\|2 | 10 | 3.33 | 0\|5\|5 | 0 | 0 | 0 | 0 | 0 | −11.39 | 1 | 0.87 |
| Q96CW1-2 | AP2M1 | 5\|3\|2 | 10 | 3.33 | 4\|2\|4 | 0 | 0 | 0 | 0 | 0 | −11.71 | 1 | 0.87 |
| Q13885 | TUBB2A | 94\|91\|96 | 281 | 93.67 | 100\|93\|91 | 0 | 0 | 0 | 0 | 0 | −204.46 | 0.99 | 0.92 |
| O00148 | DDX39A | 12\|11\|17 | 40 | 13.33 | 12\|12\|12 | 0 | 0 | 0 | 0 | 0 | −29.1 | 1.11 | 0.91 |
| Q9BW19 | KIFC1 | 2\|4\|3 | 9 | 3 | 2\|4\|3 | 0 | 0 | 0 | 0 | 0 | −10.42 | 1 | 0.87 |
| P48643 | CCTS | 25\|27\|27 | 79 | 26.33 | 35\|43\|44 | 0 | 0 | 0 | 0 | 0 | −112.92 | 0.65 | 0.92 |
| Q14807-2 | KIF22 | 2\|3\|0 | 5 | 1.67 | 5\|4\|3 | 0 | 0 | 0 | 0 | 0 | −19.11 | 0.42 | 0.92 |
| 075179-7 | ANKRD17 | 7\|10\|11 | 28 | 9.33 | 6\|11\|10 | 0 | 0 | 0 | 0 | 0 | −24.43 | 1.04 | 0.91 |
| P30041 | PRDX6 | 8\|10\|5 | 23 | 7.67 | 7\|6\|6 | 0 | 0 | 0 | 0 | 0 | −17.88 | 1.21 | 0.89 |
| Q9Y678 | COPG1 | 17\|10\|16 | 43 | 14.33 | 13\|12\|14 | 0 | 0 | 0 | 0 | 0 | −34.3 | 1.1 | 0.91 |
| Q9Y679 | AUP1 | 4\|5\|4 | 13 | 4.33 | 4\|7\|4 | 0 | 0 | 0 | 0 | 0 | −14.57 | 0.87 | 0.92 |
| P50579-3 | METAP2 | 4\|2\|3 | 9 | 3 | 3\|3\|4 | 0 | 0 | 0 | 0 | 0 | −11.71 | 0.9 | 0.88 |
| P55786 | NPEPPS | 3\|3\|4 | 10 | 3.33 | 4\|4\|4 | 0 | 0 | 0 | 0 | 0 | −12.5 | 0.83 | 0.92 |
| P37837 | TALDO1 | 2\|2\|2 | 6 | 2 | 2\|4\|2 | 0 | 0 | 0 | 0 | 0 | −9.29 | 0.75 | 0.92 |
| Q16637-4 | SMN2; | 0\|3\|3 | 6 | 2 | 4\|3\|0 | 0 | 0 | 0 | 0 | 0 | −11.76 | 0.86 | 0.85 |
| | SMN1 | | | | | | | | | | | | |
| Q9NP72 | RAB18 | 3\|4\|3 | 10 | 3.33 | 4\|4\|5 | 0 | 0 | 0 | 0 | 0 | −13.76 | 0.77 | 0.92 |
| P62879 | GNB2 | 3\|4\|4 | 11 | 3.67 | 3\|3\|5 | 0 | 0 | 0 | 0 | 0 | −11.36 | 1 | 0.88 |
| Q9NRR4-2 | DROSHA | 2\|2\|0 | 4 | 1.33 | 3\|2\|3 | 0 | 0 | 0 | 0 | 0 | −13.39 | 0.5 | 0.92 |
| Q15003 | NCAPH | 9\|9\|4 | 22 | 7.33 | 6\|5\|6 | 0 | 0 | 0 | 0 | 0 | −17.06 | 1.29 | 0.88 |
| Q9C0C2 | TNKS1BP1 | 3\|2\|3 | 8 | 2.67 | 6\|4\|4 | 0 | 0 | 0 | 0 | 0 | −16.88 | 0.57 | 0.92 |
| Q15796-2 | SMAD2 | 2\|0\|0 | 2 | 0.67 | 2\|2\|2 | 0 | 0 | 0 | 0 | 0 | −10.44 | 0.33 | 0.92 |
| Q6KB66-2 | KRT80 | 2\|0\|2 | 4 | 1.33 | 3\|2\|0 | 0 | 0 | 0 | 0 | 0 | −9.13 | 0.8 | 0.83 |
| Q9UKG1 | APPL1 | 5\|5\|6 | 16 | 5.33 | 4\|2\|5 | 0 | 0 | 0 | 0 | 0 | −8.45 | 1.45 | 0.86 |
| Q9H2G2-2 | SLK | 0\|2\|2 | 4 | 1.33 | 3\|3\|3 | 0 | 0 | 0 | 0 | 0 | −14.75 | 0.44 | 0.92 |
| Q06210-2 | GFPT1 | 21\|21\|20 | 62 | 20.6 | 18\|18\|19 | 0 | 0 | 0 | 0 | 0 | −38.38 | 1.13 | 0.92 |
| P18085 | ARF4 | 9\|8\|7 | 24 | 8 | 8\|3\|8 | 0 | 0 | 0 | 0 | 0 | −14.9 | 1.26 | 0.9 |
| O75821 | EIF3G | 2\|3\|3 | 8 | 2.67 | 2\|2\|4 | 0 | 0 | 0 | 0 | 0 | −9.29 | 1 | 0.86 |
| P30044-2 | PRDX5 | 6\|3\|5 | 14 | 4.67 | 3\|5\|4 | 0 | 0 | 0 | 0 | 0 | −12.5 | 1.17 | 0.87 |
| P78373-2 | CCT2 | 4\|0\|3 | 7 | 2.33 | 3\|3\|4 | 0 | 0 | 0 | 0 | 0 | −16.24 | 0.7 | 0.88 |
| Q9UG18-2 | TES | 12\|7\|8 | 27 | 9 | 13\|11\|10 | 0 | 0 | 0 | 0 | 0 | −33.24 | 0.79 | 0.91 |
| Q5VV41 | ARHGEF16 | 3\|8\|6 | 17 | 5.67 | 7\|6\|10 | 0 | 0 | 0 | 0 | 0 | −26.78 | 0.74 | 0.91 |
| P04222 | HLA-C | 3\|4\|4 | 11 | 3.67 | 3\|5\|3 | 0 | 0 | 0 | 0 | 0 | −11.36 | 1 | 0.88 |
| P68104 | EEF1A1 | 86\|81\|83 | 250 | 83.33 | 74\|94\|97 | 0 | 0 | 0 | 0 | 0 | −196.66 | 0.94 | 0.92 |
| P27635 | RPL10 | 21\|17\|20 | 58 | 19.33 | 21\|22\|19 | 0 | 0 | 0 | 0 | 0 | −51.18 | 0.94 | 0.92 |
| Q9UHB9-4 | SRP68 | 5\|5\|4 | 14 | 4.67 | 3\|2\|4 | 0 | 0 | 0 | 0 | 0 | −7.54 | 1.56 | 0.84 |
| Q9Y2H6-2 | FNDC3A | 4\|7\|4 | 15 | 5 | 4\|2\|9 | 0 | 0 | 0 | 0 | 0 | −14.2 | 1 | 0.89 |
| Q9Y450 | HBS1L | 6\|4\|7 | 17 | 5.67 | 8\|10\|7 | 0 | 0 | 0 | 0 | 0 | −27.32 | 0.68 | 0.92 |
| Q92804-2 | TAF15 | 25\|11\|17 | 53 | 17.67 | 15\|12\|10 | 0 | 0 | 0 | 0 | 0 | −30.3 | 1.43 | 0.89 |
| Q5BKZ1 | ZNF326 | 3\|6\|3 | 12 | 4 | 5\|2\|5 | 0 | 0 | 0 | 0 | 0 | −12.5 | 1 | 0.87 |
| Q6L807-2 | PDE12 | 5\|7\|7 | 19 | 6.33 | 7\|6\|8 | 0 | 0 | 0 | 0 | 0 | −20.31 | 0.9 | 0.92 |
| Q13151 | HNRNPAO | 8\|9\|8 | 25 | 8.33 | 7\|7\|7 | 0 | 0 | 0 | 0 | 0 | −15.83 | 1.19 | 0.9 |
| Q13155 | AIMP2 | 2\|4\|3 | 9 | 3 | 3\|3\|2 | 0 | 0 | 0 | 0 | 0 | −9.29 | 1.12 | 0.85 |
| Q14192 | FHL2 | 6\|5\|4 | 15 | 5 | 6\|5\|5 | 0 | 0 | 0 | 0 | 0 | −15.82 | 0.94 | 0.9 |
| Q86TC9 | MYPN | 5\|5\|6 | 16 | 5.33 | 3\|4\|3 | 0 | 0 | 0 | 0 | 0 | −7.29 | 1.6 | 0.84 |
| P62891 | RPL39 | 3\|3\|3 | 9 | 3 | 6\|3\|2 | 0 | 0 | 0 | 0 | 0 | −11.37 | 0.82 | 0.92 |
| Q14204 | DYNC1H1 | 26\|26\|20 | 72 | 24 | 32\|26\|26 | 0 | 0 | 0 | 0 | 0 | −73.47 | 0.86 | 0.92 |
| P62899 | RPL31 | 4\|4\|3 | 11 | 3.67 | 3\|5\|3 | 0 | 0 | 0 | 0 | 0 | −11.36 | 1 | 0.88 |
| P00338 | LDHA | 15\|11\|15 | 41 | 13.67 | 15\|15\|14 | 0 | 0 | 0 | 0 | 0 | −38.84 | 0.93 | 0.92 |
| Q92841 | DDX17 | 50\|50\|45 | 145 | 48.33 | 54\|53\|50 | 0 | 0 | 0 | 0 | 0 | −122.36 | 0.92 | 0.92 |
| P68032 | ACTC | 73\|71\|76 | 220 | 73.33 | 82\|78\|72 | 0 | 0 | 0 | 0 | 0 | −172.53 | 0.95 | 0.92 |
| P07814 | EPRS | 32\|33\|37 | 102 | 34 | 40\|30\|35 | 0 | 0 | 0 | 0 | 0 | −79.78 | 0.97 | 0.92 |
| P22102 | GART | 12\|9\|13 | 34 | 11.33 | 8\|9\|12 | 0 | 0 | 0 | 0 | 0 | −23.79 | 1.17 | 0.91 |
| O75643 | SNRNP200 | 11\|7\|7 | 25 | 8.33 | 12\|17\|14 | 0 | 0 | 0 | 0 | 0 | −44.99 | 0.58 | 0.92 |
| Q15185-4 | PTGES3 | 4\|4\|5 | 13 | 4.33 | 4\|4\|5 | 0 | 0 | 0 | 0 | 0 | −12.24 | 3 | 0.89 |
| Q8WWY3 | PRPF31 | 3\|5\|3 | 11 | 3.67 | 5\|4\|5 | 0 | 0 | 0 | 0 | 0 | −15.01 | 0.79 | 0.9 |
| P04049 | RAF1 | 3\|0\|2 | 5 | 1.67 | 2\|0\|4 | 0 | 0.01 | 0 | 0.01 | 0 | −10.2 | 0.83 | 0.83 |
| O60271-4 | ISPAG9 | 3\|3\|5 | 11 | 3.67 | 8\|6\|10 | 0 | 0 | 0 | 0 | 0 | −28.1 | 0.46 | 0.92 |
| Q9ULX6-2 | AKAPSL | 2\|2\|4 | 8 | 2.67 | 0\|4\|4 | 0 | 0 | 0 | 0 | 0 | −9.13 | 1 | 0.85 |
| Q12931-2 | TRAP1 | 22\|24\|23 | 69 | 23 | 26\|24\|29 | 0 | 0 | 0 | 0 | 0 | −63.93 | 0.87 | 0.92 |
| Q14739 | LBR | 6\|5\|8 | 19 | 6.33 | 9\|8\|7 | 0 | 0 | 0 | 0 | 0 | −24.09 | 0.79 | 0.92 |
| O75534-2 | CSDE1 | 12\|11\|13 | 36 | 12 | 12\|11\|9 | 0 | 0 | 0 | 0 | 0 | −24.4 | 1.12 | 0.91 |
| Q6UUV7-3 | CRTC3 | 2\|2\|0 | 4 | 1.33 | 6\|0\|0 | 0 | 0 | 0 | 0 | 0 | −9.65 | 0.67 | 0.92 |
| Q9UNQ2 | DIMT1 | 3\|5\|3 | 11 | 3.67 | 3\|2\|3 | 0 | 0.01 | 0 | 0.01 | 0 | −7.81 | 1.38 | 0.83 |
| O43491 | EPB41L2 | 7\|4\|3 | 14 | 4.67 | 6\|3\|7 | 0 | 0 | 0 | 0 | 0 | −17.53 | 0.88 | 0.89 |
| P11586 | MTHFD1 | 6\|7\|5 | 18 | 6 | 6\|6\|5 | 0 | 0 | 0 | 0 | 0 | −15.5 | 1.06 | 0.9 |
| P61353 | RPL27 | 12\|13\|13 | 38 | 12.67 | 14\|14\|12 | 0 | 0 | 0 | 0 | 0 | −32.37 | 0.95 | 0.92 |
| O14828 | SCAMP3 | 6\|5\|4 | 15 | 5 | 10\|8\|7 | 0 | 0 | 0 | 0 | 0 | −27.32 | 0.6 | 0.92 |
| P38919 | EIF4A3 | 19\|19\|21 | 59 | 19.67 | 18\|24\|24 | 0 | 0 | 0 | 0 | 0 | −52.87 | 0.89 | 0.92 |
| Q15645 | TRIP13 | 6\|4\|6 | 16 | 5.33 | 8\|6\|6 | 0 | 0 | 0 | 0 | 0 | −20.83 | 0.8 | 0.92 |
| Q13423 | NNT | 8\|11\|8 | 27 | 9 | 11\|9\|12 | 0 | 0 | 0 | 0 | 0 | −28.97 | 0.84 | 0.91 |
| Q7Z794 | KRT77 | 5\|5\|5 | 15 | 5 | 5\|7\|5 | 0 | 0 | 0 | 0 | 0 | −15.5 | 0.88 | 0.92 |
| P35080-2 | PFN2 | 4\|4\|4 | 12 | 4 | 4\|5\|4 | 0 | 0 | 0 | 0 | 0 | −12.24 | 0.92 | 0.92 |
| O8NI27 | THOC2 | 3\|3\|2 | 8 | 2.67 | 5\|3\|4 | 0 | 0 | 0 | 0 | 0 | −14.21 | 0.67 | 0.92 |
| Q13547 | HDAC1 | 4\|7\|9 | 20 | 6.67 | 6\|4\|3 | 0 | 0.01 | 0 | 0.01 | 0 | −12.24 | 1.54 | 0.83 |
| P48735 | IDH2 | 2\|2\|0 | 4 | 1.33 | 4\|2\|3 | 0 | 0 | 0 | 0 | 0 | −14.75 | 0.44 | 0.92 |
| O43660-2 | PLRG1 | 0\|3\|0 | 3 | 1 | 0\|2\|4 | 0 | 0.01 | 0 | 0.01 | 0 | −10.2 | 0.5 | 0.83 |
| P09884 | POLA1 | 2\|0\|0 | 2 | 0.67 | 3\|3\|0 | 0 | 0 | 0 | 0 | 0 | −10.34 | 0.33 | 0.92 |
| 015371-2 | EIF3D | 30\|27\|27 | 84 | 28 | 29\|24\|23 | 0 | 0 | 0 | 0 | 0 | −52.9 | 1.11 | 0.92 |
| Q13085-4 | ACACA | 45\|663\|67 | 1986 | 662 | 57\|672\|67 | 0 | 0 | 0 | 0 | 0 | #NAME? | 0.99 | 0.92 |
| P10768 | ESD | 10\|8\|9 | 27 | 9 | 8\|9\|9 | 0 | 0 | 0 | 0 | 0 | −21.7 | 1.04 | 0.91 |
| P84098 | RPL19 | 9\|10\|10 | 29 | 9.67 | 12\|6\|8 | 0 | 0 | 0 | 0 | 0 | −20.32 | 1.12 | 0.91 |
| Q9Y697-2 | NFS1 | 4\|3\|2 | 9 | 3 | 4\|2\|4 | 0 | 0 | 0 | 0 | 0 | −11.71 | 0.9 | 0.88 |
| P84090 | ERH | 3\|3\|3 | 9 | 3 | 4\|2\|2 | 0 | 0 | 0 | 0 | 0 | −7.81 | 1.12 | 0.86 |
| Q15269 | PWP2 | 514\|2 | 11 | 3.67 | 0\|4\|4 | 0 | 0.01 | 0 | 0.01 | 0 | −9.13 | 1.38 | 0.82 |
| P84095 | RHOG | 3\|3\|2 | 8 | 2.67 | 4\|3\|3 | 0 | 0 | 0 | 0 | 0 | −11.71 | 0.8 | 0.92 |
| P61221 | ABCE1 | 6\|6\|5 | 17 | 5.67 | 6\|5\|5 | 0 | 0 | 0 | 0 | 0 | −14.32 | 1.06 | 0.9 |
| Q13637 | RAB32 | 3\|0\|2 | 5 | 1.67 | 3\|3\|0 | 0 | 0 | 0 | 0 | 0 | −10.34 | 0.83 | 0.84 |
| P62136 | PPP1CA | 11\|10\|10 | 31 | 10.33 | 9\|9\|11 | 0 | 0 | 0 | 0 | 0 | −22.33 | 1.07 | 0.91 |
| O00487 | PSMD14 | 7\|5\|7 | 19 | 6.33 | 2\|4\|5 | 0 | 0 | 0 | 0 | 0 | −8.45 | 1.73 | 0.83 |
| P05141 | SLC25AS | 13\|11\|12 | 36 | 12 | 13\|15\|11 | 0 | 0 | 0 | 0 | 0 | −32.69 | 0.92 | 0.92 |
| O90080 | PA2G4 | 34\|37\|33 | 104 | 34.67 | 37\|36\|31 | 0 | 0 | 0 | 0 | 0 | −77.09 | 1 | 0.92 |
| P37802 | TAGLN2 | 15\|14\|13 | 42 | 14 | 19\|18\|20 | 0 | 0 | 0 | 0 | 0 | −51.69 | 0.74 | 0.92 |
| P04818-2 | TYMS | 4\|4\|3 | 11 | 3.67 | 4\|4\|3 | 0 | 0 | 0 | 0 | 0 | −11.36 | 1 | 0.88 |
| P47712 | PLA2G4A | 7\|8\|4 | 19 | 6.33 | 6\|4\|4 | 0 | 0 | 0 | 0 | 0 | −13.43 | 1.36 | 0.87 |
| Q96101 | THOC3 | 2\|3\|3 | 8 | 2.67 | 4\|3\|3 | 0 | 0 | 0 | 0 | 0 | −11.71 | 0.8 | 0.92 |
| Q9BV38 | WDR18 | 6\|4\|3 | 13 | 4.33 | 4\|6\|5 | 0 | 0 | 0 | 0 | 0 | −16.23 | 0.87 | 0.89 |
| Q96EE3 | SEH1L | 9\|8\|5 | 22 | 7.33 | 5\|7\|6 | 0 | 0 | 0 | 0 | 0 | −16.65 | 1.22 | 0.89 |
| Q9UBE0 | SAE1 | 7\|9\|7 | 23 | 7.67 | 9\|8\|9 | 0 | 0 | 0 | 0 | 0 | −23.23 | 0.88 | 0.91 |
| Q92900-2 | UPF1 | 23\|22\|19 | 64 | 21.33 | 17\|19\|19 | 0 | 0 | 0 | 0 | 0 | −39.82 | 1.16 | 0.92 |
| P13674 | P4HA1 | 3\|2\|3 | 8 | 2.67 | 2\|3\|3 | 0 | 0 | 0 | 0 | 0 | −9.29 | 3 | 0.86 |
| Q96953 | ZNF622 | 6\|5\|5 | 16 | 5.33 | 5\|6\|4 | 0 | 0 | 0 | 0 | 0 | −13.14 | 1.07 | 0.89 |
| Q16891-2 | IMMT | 5\|7\|4 | 16 | 5.33 | 4\|6\|0 | 0 | 0.01 | 0 | 0.0 | 0 | −8.27 | 1.6 | 0.82 |
| 000571 | DDX3X | 55\|59\|54 | 168 | 56 | 51\|53\|68 | 0 | 0 | 0 | 0 | 0 | −126.46 | 0.98 | 0.92 |
| P14866 | HNRNPL | 39\|34\|37 | 110 | 36.67 | 43\|43\|45 | 0 | 0 | 0 | 0 | 0 | −108.31 | 0.84 | 0.92 |
| P52594-2 | AGFG1 | 8\|5\|5 | 18 | 6 | 5\|6\|5 | 0 | 0 | 0 | 0 | 0 | −14.32 | 1.12 | 0.89 |
| P14868 | DARS | 11\|13\|13 | 37 | 12.33 | 13\|14\|10 | 0 | 0 | 0 | 0 | 0 | −30.3 | 1 | 0.91 |
| O95239 | KIF4A | 0\|2\|0 | 2 | 0.67 | 3\|2\|2 | 0 | 0 | 0 | 0 | 0 | −11.93 | 0.29 | 0.92 |
| Q96JM3 | CHAMP1 | 4\|4\|2 | 10 | 3.33 | 6\|6\|5 | 0 | 0 | 0 | 0 | 0 | −20.89 | 0.59 | 0.92 |
| O15027-5 | SEC16A | 18\|18\|23 | 59 | 19.67 | 21\|20\|21 | 0 | 0 | 0 | 0 | 0 | −49.6 | 0.95 | 0.92 |
| Q5T200-2 | ZC3H13 | 2\|0\|0 | 2 | 0.67 | 7\|5\|5 | 0 | 0 | 0 | 0 | 0 | −26.48 | 0.12 | 0.92 |
| P31947-2 | SFN | 2\|0\|2 | 4 | 1.33 | 2\|4\|2 | 0 | 0 | 0 | 0 | 0 | −13.39 | 0.5 | 0.92 |
| Q08123 | NSUN2 | 14\|13\|12 | 39 | 13 | 16\|12\|14 | 0 | 0 | 0 | 0 | 0 | −34.76 | 0.93 | 0.92 |
| P0DPH8 | PODPH8 | 73\|64\|68 | 205 | 68.33 | 69\|63\|66 | 0 | 0 | 0 | 0 | 0 | −142.55 | 1.04 | 0.92 |
| Q13470-2 | TNK1 | 6\|4\|4 | 14 | 4.67 | 9\|8\|5 | 0 | 0 | 0 | 0 | 0 | −23.39 | 0.64 | 0.92 |
| Q9Y446 | PKP3 | 7\|4\|5 | 16 | 5.33 | 5\|5\|5 | 0 | 0 | 0 | 0 | 0 | −14.57 | 1.07 | 0.89 |
| P35221 | CINNA1 | 0\|0\|2 | 2 | 0.67 | 5\|3\|0 | 0 | 0 | 0 | 0 | 0 | −13.16 | 0.25 | 0.92 |
| O60678-2 | PRMT3 | 8\|4\|4 | 16 | 5.33 | 4\|2\|5 | 0 | 0.01 | 0 | 0.01 | 0 | −9.88 | 1.45 | 0.81 |
| Q969V3-2 | NCLN | 55\|5 | 15 | 5 | 4\|5\|2 | 0 | 0 | 0 | 0 | 0 | −8.45 | 1.36 | 0.87 |
| O9Y3A5 | SBDS | 13\|15\|12 | 40 | 13.33 | 14\|13\|12 | 0 | 0 | 0 | 0 | 0 | −31.17 | 1.03 | 0.91 |
| Q9H4A3-2 | WNK1 | 5\|8\|10 | 23 | 7.67 | 7\|8\|8 | 0 | 0 | 0 | 0 | 0 | −22.85 | 3 | 0.9 |
| P42677 | RPS27 | 9\|8\|11 | 28 | 9.33 | 11\|7\|6 | 0 | 0 | 0 | 0 | 0 | −19.36 | 1.17 | 0.9 |
| P98170 | XIAP | 14\|18\|15 | 47 | 15.67 | 19\|18\|15 | 0 | 0 | 0 | 0 | 0 | −43.77 | 0.9 | 0.92 |
| Q99569-2 | PKP4 | 3\|2\|2 | 7 | 2.33 | 0\|3\|4 | 0 | 0 | 0 | 0 | 0 | −7.93 | 1 | 0.85 |
| Q04760 | GLO1 | 10\|12\|8 | 30 | 10 | 12\|11\|9 | 0 | 0 | 0 | 0 | 0 | −28.97 | 0.94 | 0.91 |
| Q5VT66-3 | 1-Mar | 5\|4\|3 | 12 | 4 | 4\|5\|3 | 0 | 0 | 0 | 0 | 0 | −12.5 | 1 | 0.88 |
| Q01082 | SPTBN1 | 6\|5\|3 | 14 | 4.67 | 7\|8\|7 | 0 | 0 | 0 | 0 | 0 | −25.42 | 0.64 | 0.92 |
| Q01081 | U2AF1 | 6\|10\|7 | 23 | 7.67 | 9\|6\|8 | 0 | 0 | 0 | 0 | 0 | −21.17 | 1 | 0.9 |
| P26373 | RPL13 | 26\|23\|27 | 76 | 25.33 | 28\|22\|23 | 0 | 0 | 0 | 0 | 0 | −55.17 | 1.04 | 0.92 |
| P42704 | LRPPRC | 4\|4\|6 | 14 | 4.67 | 4\|7\|4 | 0 | 0 | 0 | 0 | 0 | −14.57 | 0.93 | 0.89 |
| O43719 | HTATSF1 | 6\|4\|3 | 13 | 4.33 | 3\|3\|5 | 0 | 0 | 0 | 0 | 0 | −11.36 | 1.18 | 0.86 |
| 35637-2 | FUS | 5\|7\|6 | 18 | 6 | 6\|11\|11 | 0 | 0 | 0 | 0 | 0 | −29.27 | 0.64 | 0.92 |
| P59998 | ARPC4 | 3\|0\|2 | 5 | 1.67 | 2\|3\|2 | 0 | 0 | 0 | 0 | 0 | −11.93 | 0.71 | 0.86 |
| Q14687-2 | GSE1 | 7\|9\|5 | 21 | 7 | 6\|5\|7 | 0 | 0 | 0 | 0 | 0 | −16.65 | 1.17 | 0.89 |
| PS2272-2 | HNRNPM | 5\|5\|5 | 15 | 5 | 8\|8\|12 | 0 | 0 | 0 | 0 | 0 | −29.27 | 0.54 | 0.92 |
| Q9UHI6 | DDX20 | 3\|3\|2 | 8 | 2.67 | 3\|2\|3 | 0 | 0 | 0 | 0 | 0 | −9.29 | 1 | 0.86 |
| Q9H984 | SFXN1 | 6\|0\|6 | 12 | 4 | 8\|7\|7 | 0 | 0 | 0 | 0 | 0 | −33.84 | 0.55 | 0.92 |
| Q8NDV7-2 | TNRC6A | 7\|9\|14 | 30 | 10 | 8\|6\|12 | 0 | 0 | 0 | 0 | 0 | −23.29 | 1.15 | 0.9 |
| P09543-2 | CNP | 4\|4\|5 | 13 | 4.33 | 5\|4\|6 | 0 | 0 | 0 | 0 | 0 | −14.57 | 0.87 | 0.92 |
| P82675 | MRPSS | 6\|2\|3 | 11 | 3.67 | 4\|3\|4 | 0 | 0 | 0 | 0 | 0 | −12.99 | 1 | 0.86 |
| Q81X81-2 | DNAIC10 | 0\|0\|3 | 3 | 1 | 3\|4\|3 | 0 | 0 | 0 | 0 | 0 | −16.24 | 0.3 | 0.92 |
| P21291 | CSRP1 | 7\|11\|7 | 25 | 8.33 | 8\|8\|8 | 0 | 0 | 0 | 0 | 0 | −20.8 | 1.04 | 0.9 |
| P26358-2 | DNMT1 | 14\|16\|16 | 46 | 15.33 | 18\|16\|16 | 0 | 0 | 0 | 0 | 0 | −41.33 | 0.92 | 0.92 |
| Q9NYJ8 | TAB2 | 3\|2\|3 | 8 | 2.67 | 3\|0\|3 | 0 | 0 | 0 | 0 | 0 | −6.82 | 1.33 | 0.82 |
| Q8IWA0 | WDR75 | 4\|3\|5 | 12 | 4 | 4\|7\|9 | 0 | 0 | 0 | 0 | 0 | −22.76 | 0.6 | 0.92 |
| O95456-2 | PSMG1 | 6\|6\|6 | 18 | 6 | 6\|6\|8 | 0 | 0 | 0 | 0 | 0 | −17.57 | 0.9 | 0.92 |
| P46779-4 | IRPL28 | 12\|17\|15 | 44 | 14.67 | 11\|10\|12 | 0 | 0 | 0 | 0 | 0 | −24.13 | 1.33 | 0.91 |
| Q04695 | KRT17 | 38\|33\|42 | 113 | 37.67 | 41\|38\|46 | 0 | 0 | 0 | 0 | 0 | −102.54 | 0.9 | 0.92 |
| Q8N136 | WDR36 | 7\|10\|8 | 25 | 8.33 | 7\|5\|12 | 0 | 0 | 0 | 0 | 0 | −20.66 | 1.04 | 0.91 |
| Q71RC2-3 | LARP4 | 12\|13\|15 | 40 | 13.33 | 17\|13\|8 | 0 | 0 | 0 | 0 | 0 | −29.89 | 1.05 | 0.91 |
| Q96HC4-6 | \|PDLIM5 | 4\|4\|5 | 13 | 4.33 | 4\|5\|4 | 0 | 0 | 0 | 0 | 0 | −12.24 | 1 | 0.89 |
| P48729 | CSNK1A1 | 3\|2\|4 | 9 | 3 | 4\|0\|3 | 0 | 0.01 | 0 | 0.01 | 0 | −7.93 | 1.29 | 0.83 |
| Q96A08 | HIST1H2BA | 0\|2\|2 | 4 | 1.33 | 3\|2\|0 | 0 | 0 | 0 | 0 | 0 | −9.13 | 0.8 | 0.83 |
| Q8NE71-2 | \|ABCF1 | 10\|11\|12 | 33 | 11 | 13\|10\|11 | 0 | 0 | 0 | 0 | 0 | −28.23 | 0.97 | 0.91 |
| P31930 | UQCRC1 | 8\|11\|13 | 32 | 10.67 | 14\|15\|12 | 0 | 0 | 0 | 0 | 0 | −40.39 | 0.78 | 0.92 |
| P04844 | RPN2 | 13\|9\|10 | 32 | 10.67 | 11\|8\|14 | 0 | 0 | 0 | 0 | 0 | −28.56 | 0.97 | 0.91 |
| P04843 | RPN1 | 17\|17\|10 | 44 | 14.67 | 14\|15\|15 | 0 | 0 | 0 | 0 | 0 | −40.56 | 1 | 0.92 |
| Q9BYG3 | NIFK | 2\|2\|3 | 7 | 2.33 | 3\|0\|3 | 0 | 0 | 0 | 0 | 0 | −6.82 | 1.17 | 0.84 |
| P33981-2 | TTK | 3\|3\|3 | 9 | 3 | 2\|0\|5 | 0 | 0 | 0 | 0 | 0 | −6.2 | 1.29 | 0.83 |
| Q15366-6 | PCBP2 | 25\|27\|25 | 77 | 25.67 | 22\|25\|27 | 0 | 0 | 0 | 0 | 0 | −53.44 | 1.04 | 0.92 |
| Q9P2E9 | RRBP1 | 8\|9\|11 | 28 | 9.33 | 10\|9\|10 | 0 | 0 | 0 | 0 | 0 | −25.3 | 0.97 | 0.91 |
| P61254 | RPL26 | 11\|13\|12 | 36 | 12 | 16\|11\|13 | 0 | 0 | 0 | 0 | 0 | −33.92 | 0.9 | 0.92 |
| P68431 | HIST1H3G; | 2\|2\|2 | 6 | 2 | 3\|2\|2 | 0 | 0 | 0 | 0 | 0 | −8.1 | 0.86 | 0.92 |
| | HIST1H3 | | | | | | | | | | | | |
| Q15459 | SF3A1 | 42\|40\|39 | 121 | 40.33 | 44\|37\|47 | 0 | 0 | 0 | 0 | 0 | −96.68 | 0.95 | 0.92 |
| Q9H0J9 | PARP12 | 2\|0\|0 | 2 | 0.67 | 3\|2\|0 | 0 | 0 | 0 | 0 | 0 | −9.13 | 0.4 | 0.84 |
| Q96EY1-2 | DNAJA3 | 4\|5\|5 | 14 | 4.67 | 5\|7\|5 | 0 | 0 | 0 | 0 | 0 | −17.06 | 0.82 | 0.92 |
| P55072 | VCP | 9\|8\|9 | 26 | 8.67 | 8\|4\|4 | 0 | 0 | 0 | 0 | 0 | −10.02 | 1.62 | 0.87 |
| P51571 | SSR4 | 3\|2\|3 | 8 | 2.67 | 3\|4\|3 | 0 | 0 | 0 | 0 | 0 | −11.71 | 0.8 | 0.92 |
| PS1572 | BCAP31 | 8\|9\|5 | 22 | 7.33 | 5\|7\|7 | 0 | 0 | 0 | 0 | 0 | −17.88 | 1.16 | 0.9 |
| P17706-2 | PTPN2 | 2\|3\|3 | 8 | 2.67 | 3\|0\|3 | 0 | 0 | 0 | 0 | 0 | −6.82 | 1.33 | 0.82 |
| P06737-2 | PYGL | 3\|4\|5 | 12 | 4 | 6\|7\|5 | 0 | 0 | 0 | 0 | 0 | −20.1 | 0.67 | 0.92 |
| P13861-2 | PRKAR2A | 0\|5\|4 | 9 | 3 | 3\|6\|7 | 0 | 0 | 0 | 0 | 0 | −25 | 0.56 | 0.92 |
| Q9BYD1 | MRPL13 | 2\|3\|3 | 8 | 2.67 | 2\|3\|3 | 0 | 0 | 0 | 0 | 0 | −9.29 | 1 | 0.86 |
| P13645 | KRT10 | 40\|28\|32 | 100 | 33.33 | 38\|58\|36 | 0 | 0 | 0 | 0 | 0 | −119.67 | 0.76 | 0.92 |
| P13647 | KRTS | 19\|13\|15 | 47 | 15.67 | 18\|17\|11 | 0 | 0 | 0 | 0 | 0 | −38.05 | 1.02 | 0.92 |
| Q9BTE3-2 | MCMBP | 4\|5\|4 | 13 | 4.33 | 6\|6\|4 | 0 | 0 | 0 | 0 | 0 | −15.82 | 0.81 | 0.92 |
| Q07020 | RPL18 | 12\|14\|11 | 37 | 12.33 | 14\|14\|14 | 0 | 0 | 0 | 0 | 0 | −36.35 | 0.88 | 0.92 |
| Q9NSE4 | LARS2 | 18\|13\|13 | 44 | 14.67 | 14\|18\|15 | 0 | 0 | 0 | 0 | 0 | −39.27 | 0.94 | 0.92 |
| Q9NYF8-3 | BCLAF1 | 0\|2\|0 | 2 | 0.67 | 4\|2\|4 | 0 | 0 | 0 | 0 | 0 | −16.24 | 0.2 | 0.92 |
| Q03252 | LMNB2 | 5\|4\|3 | 12 | 4 | 3\|5\|3 | 0 | 0 | 0 | 0 | 0 | −11.36 | 1.09 | 0.88 |
| P17612 | PRKACA | 5\|7\|5 | 17 | 5.67 | 6\|4\|4 | 0 | 0 | 0 | 0 | 0 | −11.96 | 1.21 | 0.88 |
| Q5SW79 | CEP170 | 7\|4\|5 | 16 | 5.33 | 7\|6\|10 | 0 | 0 | 0 | 0 | 0 | −24.7 | 0.7 | 0.92 |
| Q04917 | YWHAH | 6\|4\|3 | 13 | 4.33 | 4\|5\|9 | 0 | 0 | 0 | 0 | 0 | −20.13 | 0.72 | 0.92 |
| Q9Y230 | RUVBL2 | 13\|13\|13 | 39 | 13 | 17\|15\|18 | 0 | 0 | 0 | 0 | 0 | −42.94 | 0.78 | 0.92 |
| Q92974-3 | ARHGEF2 | 9\|11\|12 | 32 | 10.67 | 8\|11\|8 | 0 | 0 | 0 | 0 | 0 | −21.43 | 1.19 | 0.91 |
| P10515 | DLAT | 13\|14\|9 | 36 | 12 | 13\|13\|13 | 0 | 0 | 0 | 0 | 0 | −35.99 | 0.92 | 0.92 |
| Q9Y606-2 | PUS1 | 2\|3\|2 | 7 | 2.33 | 0\|5\|3 | 0 | 0 | 0 | 0 | 0 | −9.07 | 0.88 | 0.87 |
| P08237 | PFKM | 14\|20\|19 | 53 | 17.67 | 18\|18\|17 | 0 | 0 | 0 | 0 | 0 | −45 | 1 | 0.92 |
| P18754 | RCC3 | 11\|8\|9 | 28 | 9.33 | 8\|8\|8 | 0 | 0 | 0 | 0 | 0 | −19.36 | 1.17 | 0.9 |
| P08238 | HSP90AB1 | 97\|90\|111 | 298 | 99.33 | 11\|111\|10 | 0 | 0 | 0 | 0 | 0 | −262.84 | 0.9 | 0.92 |
| Q7L014 | DDX46 | 3\|5\|4 | 12 | 4 | 16\|14\|8 | 0 | 0 | 0 | 0 | 0 | −47.47 | 0.32 | 0.92 |
| Q1KMD3 | HNRNPUL2 | 0\|4\|4 | 8 | 2.67 | 5\|5\|3 | 0 | 0 | 0 | 0 | 0 | −20.61 | 0.62 | 0.92 |
| P58876 | HIST1H2BD | 4\|5\|6 | 15 | 5 | 9\|8\|5 | 0 | 0 | 0 | 0 | 0 | −23.39 | 0.68 | 0.92 |
| O15226 | NKRF | 0\|3\|2 | 5 | 1.67 | 5\|3\|0 | 0 | 0 | 0 | 0 | 0 | −13.16 | 0.63 | 0.87 |
| Q9P215 | LARS | 17\|14\|15 | 46 | 15.33 | 17\|11\|19 | 0 | 0 | 0 | 0 | 0 | −37.77 | 0.98 | 0.92 |
| P28340 | POLD1 | 5\|3\|4 | 12 | 4 | 3\|4\|3 | 0 | 0 | 0 | 0 | 0 | −10.17 | 1.2 | 0.86 |
| P35237 | SERPINB6 | 6\|6\|9 | 21 | 7 | 8\|4\|7 | 0 | 0 | 0 | 0 | 0 | −16.39 | 1.11 | 0.9 |
| P35232 | PHB | 3\|2\|2 | 7 | 2.33 | 5\|3\|2 | 0 | 0 | 0 | 0 | 0 | −11.71 | 0.7 | 0.92 |
| P30876 | POLR2B | 6\|6\|5 | 17 | 5.67 | 4\|6\|6 | 0 | 0 | 0 | 0 | 0 | −14.32 | 1.06 | 0.9 |
| P15924 | DSP | 28\|24\|29 | 81 | 27 | 28\|30\|26 | 0 | 0 | 0 | 0 | 0 | −66.84 | 0.96 | 0.92 |
| P52948-6 | NUP98 | 17\|20\|18 | 55 | 18.33 | 19\|22\|25 | 0 | 0 | 0 | 0 | 0 | −56.09 | 0.83 | 0.92 |
| Q81X12-2 | CCAR1 | 0\|2\|3 | 5 | 1.67 | 3\|0\|4 | 0 | 0 | 0 | 0 | 0 | −11.76 | 0.71 | 0.85 |
| O95071-2 | UBRS | 7\|4\|4 | 15 | 5 | 12\|8\|7 | 0 | 0 | 0 | 0 | 0 | −29.96 | 0.56 | 0.92 |
| Q9Y3F4 | STRAP | 7\|7\|6 | 20 | 6.67 | 6\|7\|6 | 0 | 0 | 0 | 0 | 0 | −16.39 | 1.05 | 0.9 |
| P17980 | PSMC3 | 8\|11\|8 | 27 | 9 | 8\|9\|6 | 0 | 0 | 0 | 0 | 0 | −18.18 | 1.17 | 0.9 |
| P17987 | TCP1 | 42\|41\|44 | 127 | 42.33 | 44\|41\|48 | 0 | 0 | 0 | 0 | 0 | −99.62 | 0.95 | 0.92 |
| P06239 | LCK | 3\|0\|4 | 7 | 2.33 | 3\|3\|2 | 0 | 0 | 0 | 0 | 0 | −13.39 | 0.88 | 0.85 |
| P63220 | RPS21 | 4\|5\|3 | 12 | 4 | 3\|4\|4 | 0 | 0 | 0 | 0 | 0 | −11.36 | 1.09 | 0.88 |
| O43143 | DHX15 | 19\|17\|14 | 50 | 16.67 | 14\|21\|19 | 0 | 0 | 0 | 0 | 0 | −46.22 | 0.93 | 0.92 |
| P67775 | PPP2CA | 3\|5\|5 | 13 | 4.33 | 5\|7\|6 | 0 | 0 | 0 | 0 | 0 | −20.1 | 0.72 | 0.92 |
| O75663 | TIPRL | 2\|3\|0 | 5 | 1.67 | 3\|0\|4 | 0 | 0 | 0 | 0 | 0 | −11.76 | 0.71 | 0.85 |
| P53597 | SUCLG1 | 2\|2\|4 | 8 | 2.67 | 2\|2\|3 | 0 | 0.01 | 0 | 0.01 | 0 | −8.1 | 1.14 | 0.84 |
| Q6PI48 | DARS2 | 6\|5\|7 | 18 | 6 | 7\|9\|7 | 0 | 0 | 0 | 0 | 0 | −22.85 | 0.78 | 0.92 |
| O43709 | BUD23 | 7\|6\|6 | 19 | 6.33 | 4\|5\|4 | 0 | 0 | 0 | 0 | 0 | −9.36 | 1.46 | 0.87 |
| P84103-2 | ISRSF3 | 2\|0\|3 | 5 | 1.67 | 2\|0\|5 | 0 | 0 | 0 | 0 | 0 | −11.59 | 0.71 | 0.85 |
| P42285 | SKIV2L2 | 5\|8\|8 | 21 | 7 | 8\|6\|5 | 0 | 0 | 0 | 0 | 0 | −17.88 | 1.11 | 0.9 |
| P62424 | RPLZA | 22\|21\|23 | 66 | 22 | 30\|24\|22 | 0 | 0 | 0 | 0 | 0 | −61.87 | 0.87 | 0.92 |
| P28331-3 | NDUFS1 | 5\|4\|5 | 14 | 4.67 | 4\|3\|3 | 0 | 0 | 0 | 0 | 0 | −8.71 | 1.4 | 0.86 |
| P07900 | HSP90AA1 | 43\|44\|48 | 135 | 45 | 56\|55\|55 | 0 | 0 | 0 | 0 | 0 | −136.68 | 0.81 | 0.92 |
| Q14152 | EIF3A | 2\|3\|7 | 1.2 | 4 | 12\|7\|4 | 0 | 0 | 0 | 0 | 0 | −28.89 | 0.52 | 0.92 |
| Q14151 | SAFB2 | 4\|5\|4 | 13 | 4.33 | 13\|10\|13 | 0 | 0 | 0 | 0 | 0 | −42.17 | 0.36 | 0.92 |
| P26038 | MSN | 2\|2\|0 | 4 | 1.33 | 2\|3\|2 | 0 | 0 | 0 | 0 | 0 | −11.93 | 0.57 | 0.92 |
| Q9Y5J1 | UTP18 | 2\|0\|2 | 4 | 1.33 | 3\|3\|3 | 0 | 0 | 0 | 0 | 0 | −14.75 | 0.44 | 0.92 |
| P19623 | SRM | 5\|8\|5 | 18 | 6 | 4\|3\|5 | 0 | 0 | 0 | 0 | 0 | −9.61 | 1.5 | 0.84 |
| P15121 | AKR181 | 4\|5\|4 | 13 | 4.33 | 4\|3\|3 | 0 | 0 | 0 | 0 | 0 | −8.71 | 1.3 | 0.86 |
| P34897-3 | SHMT2 | 5\|6\|7 | 18 | 6 | 5\|7\|7 | 0 | 0 | 0 | 0 | 0 | −17.88 | 0.95 | 0.9 |
| P63241 | EIFSA | 9\|4\|8 | 21 | 7 | 8\|6\|6 | 0 | 0 | 0 | 0 | 0 | −20.83 | 1.05 | 0.9 |
| P63244 | RACK1 | 15\|13\|15 | 43 | 14.33 | 11\|14\|15 | 0 | 0 | 0 | 0 | 0 | −30.9 | 1.07 | 0.92 |
| Q13443 | ADAM9 | 4\|6\|6 | 16 | 5.33 | 5\|2\|3 | 0 | 0 | 0 | 0 | 0 | −8.71 | 1.6 | 0.83 |
| P62333 | PSMC6 | 8\|6\|5 | 19 | 6.33 | 10\|5\|9 | 0 | 0 | 0 | 0 | 0 | −24.09 | 0.79 | 0.92 |
| Q16576 | RBBP7 | 6\|6\|5 | 17 | 5.67 | 8\|7\|6 | 0 | 0 | 0 | 0 | 0 | −20.31 | 0.81 | 0.92 |
| P26639 | TARS | 34\|27\|38 | 99 | 33 | 37\|37\|34 | 0 | 0 | 0 | 0 | 0 | −91.49 | 0.92 | 0.92 |
| Q9NZB2 | FAM120A | 7\|8\|10 | 25 | 8.33 | 8\|8\|5 | 0 | 0 | 0 | 0 | 0 | −17.28 | 1.19 | 0.9 |
| P30479 | HLA-B | 3\|3\|3 | 9 | 3 | 5\|3\|2 | 0 | 0 | 0 | 0 | 0 | −10.17 | 0.9 | 0.92 |
| P13804-2 | ETFA | 5\|5\|3 | 13 | 4.33 | 5\|7\|7 | 0 | 0 | 0 | 0 | 0 | −21.43 | 0.68 | 0.92 |
| O60566 | BUB1B | 9\|9\|13 | 31 | 10.33 | 8\|11\|9 | 0 | 0 | 0 | 0 | 0 | −22.61 | 1.11 | 0.91 |
| Q9UBU9 | NXF1 | 11\|9\|9 | 29 | 9.67 | 8\|6\|10 | 0 | 0 | 0 | 0 | 0 | −17.91 | 1.21 | 0.9 |
| P30475 | HLA-B | 3\|4\|3 | 10 | 3.33 | 3\|4\|3 | 0 | 0 | 0 | 0 | 0 | −10.17 | 1 | 0.88 |
| Q13509 | TU883 | 54\|62\|65 | 181 | 60.33 | 62\|61\|59 | 0 | 0 | 0 | 0 | 0 | −138.54 | 0.99 | 0.92 |
| P61026 | RAB10 | 7\|7\|9 | 23 | 7.67 | 9\|9\|12 | 0 | 0 | 0 | 0 | 0 | −28.15 | 0.77 | 0.92 |
| P61020 | RAB58 | 6\|4\|4 | 14 | 4.67 | 6\|6\|7 | 0 | 0 | 0 | 0 | 0 | −19.55 | 0.74 | 0.92 |
| Q16719 | KYNU | 3\|3\|2 | 8 | 2.67 | 2\|2\|3 | 0 | 0 | 0 | 0 | 0 | −8.1 | 1.14 | 0.85 |
| Q7L576 | CYFIP1 | 4\|3\|4 | 11 | 3.67 | 3\|3\|3 | 0 | 0 | 0 | 0 | 0 | −8.99 | 1.22 | 0.86 |
| Q9UQE7 | SMC3 | 14\|11\|15 | 40 | 13.33 | 15\|12\|14 | 0 | 0 | 0 | 0 | 0 | −35.14 | 0.98 | 0.92 |
| P48634 | PRRC2A | 6\|6\|6 | 18 | 6 | 6\|4\|7 | 0 | 0 | 0 | 0 | 0 | −14.03 | 1.06 | 0.9 |
| Q96KR1 | ZFR | 14\|13\|13 | 40 | 13.33 | 17\|14\|12 | 0 | 0 | 0 | 0 | 0 | −34.44 | 0.93 | 0.92 |
| Q15286 | RAB35 | 4\|5\|6 | 15 | 5 | 5\|6\|4 | 0 | 0 | 0 | 0 | 0 | −14.57 | 3 | 0.89 |
| P61247 | RPS3A | 12\|9\|12 | 33 | 11 | 12\|12\|14 | 0 | 0 | 0 | 0 | 0 | −34.75 | 0.87 | 0.92 |
| Q13619 | CUL4A | 5\|6\|5 | 16 | 5.33 | 9\|10\|11 | 0 | 0 | 0 | 0 | 0 | −31.87 | 0.53 | 0.92 |
| P15313 | EZR | 6\|6\|8 | 20 | 6.67 | 3\|5\|4 | 0 | 0 | 0 | 0 | 0 | −8.2 | 1.67 | 0.84 |
| P60866 | RPS20 | 5\|3\|6 | 14 | 4.67 | 6\|4\|3 | 0 | 0 | 0 | 0 | 0 | −13.76 | 1.08 | 0.88 |
| Q9BQC3-2 | DPH2 | 0\|0\|2 | 2 | 0.67 | 2\|0\|3 | 0 | 0 | 0 | 0 | 0 | −9.13 | 0.4 | 0.84 |
| O00165-5 | HAX1 | 3\|4\|5 | 12 | 4 | 3\|3\|3 | 0 | 0 | 0 | 0 | 0 | −8.99 | 1.33 | 0.85 |
| P56537 | EIF6 | 8\|6\|4 | 18 | 6 | 6\|6\|6 | 0 | 0 | 0 | 0 | 0 | −18.27 | 1 | 0.9 |
| O14776-2 | TCERG1 | 0\|0\|2 | 2 | 0.67 | 2\|3\|0 | 0 | 0 | 0 | 0 | 0 | −9.13 | 0.4 | 0.84 |
| Q658J3 | POTEE | 22\|20\|22 | 64 | 21.33 | 22\|24\|19 | 0 | 0 | 0 | 0 | 0 | −50.15 | 0.98 | 0.92 |
| P29353 | SHC1 | 9\|8\|8 | 25 | 8.33 | 7\|6\|7 | 0 | 0 | 0 | 0 | 0 | −14.67 | 1.25 | 0.9 |
| Q12904 | AIMP1 | 4\|3\|2 | 9 | 3 | 5\|3\|5 | 0 | 0 | 0 | 0 | 0 | −15.55 | 0.69 | 0.92 |
| P49419-2 | ALDH7A1 | 6\|2\|5 | 13 | 4.33 | 7\|3\|5 | 0 | 0 | 0 | 0 | 0 | −18.16 | 0.87 | 0.89 |
| Q9UJW0 | DCTN4 | 3\|5\|3 | 11 | 3.67 | 4\|4\|5 | 0 | 0 | 0 | 0 | 0 | −13.76 | 0.85 | 0.89 |
| Q9BXY0 | MAK16 | 2\|0\|0 | 2 | 0.67 | 2\|2\|2 | 0 | 0 | 0 | 0 | 0 | −10.44 | 0.33 | 0.92 |
| Q9P035 | HACD3 | 0\|3\|2 | 5 | 1.67 | 3\|3\|3 | 0 | 0 | 0 | 0 | 0 | −14.75 | 0.56 | 0.92 |
| Q00341-2 | HDLBP | 6\|6\|7 | 19 | 6.33 | 7\|5\|6 | 0 | 0 | 0 | 0 | 0 | −15.21 | 1.06 | 0.9 |
| P27816 | MAP4 | 25\|20\|21 | 66 | 22 | 36\|33\|26 | 0 | 0 | 0 | 0 | 0 | −87.49 | 0.69 | 0.92 |
| Q12874 | SF3A3 | 15\|14\|14 | 43 | 14.33 | 15\|13\|16 | 0 | 0 | 0 | 0 | 0 | −34.15 | 0.98 | 0.92 |
| Q14203-3 | DCTN1 | 14\|17\|20 | 51 | 17 | 16\|13\|13 | 0 | 0 | 0 | 0 | 0 | −31.8 | 1.21 | 0.91 |
| Q13363-2 | CTBP1 | 3\|3\|2 | 8 | 2.67 | 4\|2\|3 | 0 | 0 | 0 | 0 | 0 | −10.42 | 0.89 | 0.92 |
| P51532-5 | SMARCA4 | 2\|4\|3 | 9 | 3 | 3\|4\|2 | 0 | 0 | 0 | 0 | 0 | −10.42 | 1 | 0.87 |
| Q93052 | LPP | 11\|13\|12 | 36 | 12 | 14\|8\|9 | 0 | 0 | 0 | 0 | 0 | −23.23 | 1.16 | 0.91 |
| P08754 | GNA13 | 5\|5\|3 | 13 | 4.33 | 6\|6\|7 | 0 | 0 | 0 | 0 | 0 | −21.43 | 0.68 | 0.92 |
| P31327 | CPS1 | 12\|109\|11 | 332 | 110.67 | 31\|130\|12 | 0 | 0 | 0 | 0 | 0 | −300.74 | 0.86 | 0.92 |
| P62937 | PPIA | 12\|12\|8 | 32 | 10.67 | 13\|16\|15 | 0 | 0 | 0 | 0 | 0 | −44.29 | 0.73 | 0.92 |
| P09622 | DLD | 12\|10\|9 | 31 | 10.33 | 10\|8\|9 | 0 | 0 | 0 | 0 | 0 | −21.43 | 1.15 | 0.91 |
| O43865 | AHCYL | 11\|6\|8 | 25 | 8.33 | 7\|8\|11 | 0 | 0 | 0 | 0 | 0 | −24.88 | 0.96 | 0.91 |
| P36776-2 | LONP1 | 5\|5\|8 | 18 | 6 | 6\|8\|11 | 0 | 0 | 0 | 0 | 0 | −25.39 | 0.72 | 0.92 |
| Q9Y314 | NOSIP | 7\|7\|6 | 20 | 6.67 | 6\|6\|5 | 0 | 0 | 0 | 0 | 0 | −14.03 | 1.18 | 0.9 |
| P46776 | RPL27A | 5\|6\|8 | 19 | 6.33 | 10\|5\|9 | 0 | 0 | 0 | 0 | 0 | −24.09 | 0.79 | 0.92 |
| Q8WXF1 | PSPC1 | 4\|2\|6 | 12 | 4 | 2\|3\|5 | 0 | 0 | 0 | 0 | 0 | −11.71 | 1.2 | 0.85 |
| P46778 | RPL21 | 25\|19\|20 | 64 | 21.33 | 29\|24\|21 | 0 | 0 | 0 | 0 | 0 | −62.68 | 0.86 | 0.92 |
| O60701-2 | UGDH | 8\|8\|6 | 22 | 7.33 | 6\|6\|7 | 0 | 0 | 0 | 0 | 0 | −16.39 | 1.16 | 0.9 |
| P43897 | TSFM | 2\|5\|7 | 14 | 4.67 | 4\|3\|7 | 0 | 0 | 0 | 0 | 0 | −16.88 | 3 | 0.88 |
| P52209-2 | PGD | 4\|2\|4 | 10 | 3.33 | 3\|2\|7 | 0 | 0 | 0 | 0 | 0 | −14.04 | 0.83 | 0.92 |
| Q3ZCQ8 | TIMM50 | 4\|4\|3 | 11 | 3.67 | 4\|6\|5 | 0 | 0 | 0 | 0 | 0 | −16.23 | 0.73 | 0.92 |
| P34896-2 | SHMT1 | 5\|4\|2 | 11 | 3.67 | 3\|4\|4 | 0 | 0 | 0 | 0 | 0 | −12.99 | 3 | 0.88 |
| Q6P2Q9 | PRPF8 | 19\|22\|21 | 62 | 20.67 | 30\|35\|35 | 0 | 0 | 0 | 0 | 0 | −95.88 | 0.62 | 0.92 |
| Q9H7E2-3 | TDRD3 | 3\|6\|3 | 12 | 4 | 6\|4\|5 | 0 | 0 | 0 | 0 | 0 | −16.23 | 0.8 | 0.89 |
| O75874 | IDH1 | 18\|16\|15 | 49 | 16.33 | 19\|14\|16 | 0 | 0 | 0 | 0 | 0 | −38.59 | 3 | 0.92 |
| Q92878 | RAD50 | 3\|4\|7 | 14 | 4.67 | 11\|7\|5 | 0 | 0 | 0 | 0 | 0 | −26.78 | 0.61 | 0.92 |
| Q15942 | ZYX | 4\|4\|7 | 15 | 5 | 6\|7\|6 | 0 | 0 | 0 | 0 | 0 | −19.55 | 0.79 | 0.9 |
| Q9Y3U8 | RPL36 | 4\|3\|4 | 11 | 3.67 | 2\|3\|4 | 0 | 0 | 0 | 0 | 0 | −8.99 | 1.22 | 0.86 |
| Q68CZ2 | TNS3 | 8\|4\|5 | 17 | 5.67 | 3\|4\|7 | 0 | 0 | 0 | 0 | 0 | −13.43 | 1.21 | 0.87 |
| Q14527 | HLTE | 3\|3\|4 | 10 | 3.33 | 2\|4\|3 | 0 | 0 | 0 | 0 | 0 | −8.99 | 1.11 | 0.87 |
| P62753 | RPS6 | 14\|16\|16 | 46 | 15.33 | 19\|16\|20 | 0 | 0 | 0 | 0 | 0 | −47.47 | 0.84 | 0.92 |
| P62750 | RPL23A | 6\|8\|9 | 23 | 7.67 | 11\|8\|9 | 0 | 0 | 0 | 0 | 0 | −27.4 | 0.82 | 0.92 |
| P22392 | NME2 | 2\|2\|2 | 6 | 2 | 3\|2\|2 | 0 | 0 | 0 | 0 | 0 | −8.1 | 0.86 | 0.92 |
| P04075 | ALDOA | 14\|15\|15 | 44 | 14.67 | 13\|13\|16 | 0 | 0 | 0 | 0 | 0 | −31.8 | 1.05 | 0.92 |
| P09104-2 | JENO2 | 0\|0\|2 | 2 | 0.67 | 0\|3\|5 | 0 | 0 | 0 | 0 | 0 | −13.16 | 0.25 | 0.92 |
| Q6UB35 | MTHFD1L | 7\|6\|6 | 19 | 6.33 | 5\|7\|6 | 0 | 0 | 0 | 0 | 0 | −15.21 | 1.06 | 0.9 |
| P00761 | P00761 | 28\|27\|24 | 79 | 26.33 | 31\|24\|25 | 0 | 0 | 0 | 0 | 0 | −62.02 | 0.99 | 0.92 |
| P41250 | GARS | 17\|20\|21 | 58 | 19.3 | 24\|29\|33 | 0 | 0 | 0 | 0 | 0 | −81.53 | 0.67 | 0.92 |
| P41252 | LARS | 5\|9\|10 | 24 | 8 | 12\|11\|15 | 0 | 0 | 0 | 0 | 0 | −42.6 | 0.63 | 0.92 |
| Q9YSY2 | NUBP2 | 3\|4\|2 | 9 | 3 | 6\|0\|5 | 0 | 0 | 0 | 0 | 0 | −12.66 | 0.82 | 0.88 |
| Q14697-2 | GANAB | 19\|21\|16 | 56 | 18.67 | 17\|17\|16 | 0 | 0 | 0 | 0 | 0 | −38.3 | 1.12 | 0.92 |
| Q13813-3 | SPTAN1 | 0\|5\|5 | 10 | 3.33 | 10\|11\|12 | 0 | 0 | 0 | 0 | 0 | −50.12 | 0.3 | 0.92 |
| Q9H487 | TUBB1 | 10\|11\|11 | 32 | 10.67 | 13\|11\|10 | 0 | 0 | 0 | 0 | 0 | −28.23 | 0.94 | 0.92 |
| P07355 | ANXA2 | 6\|6\|7 | 19 | 6.33 | 6\|5\|5 | 0 | 0 | 0 | 0 | 0 | −12.86 | 1.19 | 0.89 |
| P46821 | MAP1B | 5\|6\|3 | 14 | 4.67 | 7\|4\|9 | 0 | 0 | 0 | 0 | 0 | −22.76 | 0.7 | 0.92 |
| Q9H0A0 | NAT10 | 21\|18\|20 | 59 | 19.67 | 15\|19\|18 | 0 | 0 | 0 | 0 | 0 | −37.75 | 1.13 | 0.92 |
| O60341 | KDM1A | 3\|3\|3 | 9 | 3 | 4\|4\|2 | 0 | 0 | 0 | 0 | 0 | −10.17 | 0.9 | 0.92 |
| Q9NV06 | DCAF13 | 4\|4\|3 | 11 | 3.67 | 4\|4\|0 | 0 | 0 | 0 | 0 | 0 | −7.55 | 1.38 | 0.84 |
| P19367-4 | HK1 | 0\|2\|0 | 2 | 0.67 | 2\|2\|3 | 0 | 0 | 0 | 0 | 0 | −11.93 | 0.29 | 0.92 |
| P61019 | RAB2A | 6\|6\|6 | 18 | 6 | 4\|7\|7 | 0 | 0 | 0 | 0 | 0 | −15.21 | 3 | 0.92 |
| Q6DK11 | RPL7L1 | 4\|3\|3 | 10 | 3.33 | 3\|2\|3 | 0 | 0 | 0 | 0 | 0 | −7.81 | 1.25 | 0.85 |
| Q9NW82 | WDR70 | 2\|3\|3 | 8 | 2.67 | 4\|2\|3 | 0 | 0 | 0 | 0 | 0 | −10.42 | 0.89 | 0.92 |
| P58107 | EPPK1 | 25\|18\|23 | 66 | 22 | 32\|38\|32 | 0 | 0 | 0 | 0 | 0 | −100.51 | 0.65 | 0.92 |
| P07910-2 | HNRNPC | 6\|7\|6 | 19 | 6.33 | 12\|13\|8 | 0 | 0 | 0 | 0 | 0 | −33.83 | 0.58 | 0.92 |
| Q13618-2 | CUL3 | 5\|4\|4 | 13 | 4.33 | 6\|7\|4 | 0 | 0 | 0 | 0 | 0 | −17.06 | 0.76 | 0.92 |
| P36542 | ATP5C1 | 8\|4\|8 | 20 | 6.67 | 7\|8\|12 | 0 | 0 | 0 | 0 | 0 | −29.96 | 0.74 | 0.92 |
| Q66PJ3-7 | ARL6IP4 | 2\|2\|2 | 6 | 2 | 2\|2\|3 | 0 | 0 | 0 | 0 | 0 | −8.1 | 0.86 | 0.92 |
| Q9NR30 | DDX21 | 32\|35\|34 | 101 | 33.67 | 33\|30\|35 | 0 | 0 | 0 | 0 | 0 | −71.48 | 1.03 | 0.92 |
| P52789 | HK2 | 6\|4\|6 | 16 | 5.33 | 6\|5\|5 | 0 | 0 | 0 | 0 | 0 | −15.82 | 3 | 0.9 |
| P49736 | MCM2 | 11\|8\|12 | 31 | 10.33 | 10\|12\|12 | 0 | 0 | 0 | 0 | 0 | −31.46 | 0.91 | 0.91 |
| Q96QD8 | SLC38A2 | 0\|0\|2 | 2 | 0.67 | 2\|2\|2 | 0 | 0 | 0 | 0 | 0 | −10.44 | 0.33 | 0.92 |
| Q96L92 | SNX27 | 3\|3\|4 | 10 | 3.33 | 4\|2\|4 | 0 | 0 | 0 | 0 | 0 | −10.17 | 1 | 0.88 |
| Q9Y2L1 | DIS3 | 7\|9\|9 | 25 | 8.33 | 14\|10\|8 | 0 | 0 | 0 | 0 | 0 | −30.69 | 0.78 | 0.92 |
| Q92621 | NUP205 | 2\|5\|4 | 11 | 3.67 | 2\|3\|3 | 0 | 0.01 | 0 | 0.01 | 0 | −9.29 | 1.38 | 0.83 |
| Q15436 | SEC23A | 8\|4\|3 | 15 | 5 | 4\|4\|6 | 0 | 0 | 0 | 0 | 0 | −15.01 | 1.07 | 0.87 |
| P30466 | HLA-B | 3\|3\|3 | 9 | 3 | 4\|3\|2 | 0 | 0 | 0 | 0 | 0 | −8.99 | 1 | 0.92 |
| P30464 | HLA-B | 3\|4\|3 | 10 | 3.33 | 3\|4\|3 | 0 | 0 | 0 | 0 | 0 | −10.17 | 3 | 0.88 |
| P09960 | LTA4H | 0\|3\|3 | 6 | 2 | 3\|2\|4 | 0 | 0 | 0 | 0 | 0 | −14.75 | 0.67 | 0.92 |
| Q6NVY1 | HIBCH | 0\|2\|0 | 2 | 0.67 | 0\|3\|2 | 0 | 0 | 0 | 0 | 0 | −9.13 | 0.4 | 0.84 |
| P30084 | ECHS1 | 2\|0\|0 | 2 | 0.67 | 2\|2\|2 | 0 | 0 | 0 | 0 | 0 | −10.44 | 0.33 | 0.92 |
| O15027 | SEC16A | 18\|16\|23 | 57 | 19 | 20\|19\|22 | 0 | 0 | 0 | 0 | 0 | −51.57 | 0.93 | 0.92 |
| P20340 | RAB6A | 11\|12\|10 | 33 | 11 | 9\|10\|11 | 0 | 0 | 0 | 0 | 0 | −23.5 | 1.1 | 0.91 |
| Q0VDF9 | HSPA14 | 4\|2\|4 | 10 | 3.33 | 3\|5\|3 | 0 | 0 | 0 | 0 | 0 | −12.99 | 0.91 | 0.88 |
| O75746-2 | SLC25A12 | 6\|7\|4 | 17 | 5.67 | 6\|3\|5 | 0 | 0 | 0 | 0 | 0 | −13.43 | 1.21 | 0.88 |
| Q6P587 | FAHD1 | 3\|3\|2 | 8 | 2.67 | 3\|2\|3 | 0 | 0 | 0 | 0 | 0 | −9.29 | 1 | 0.86 |
| P23921 | RRM1 | 18\|16\|16 | 50 | 16.67 | 14\|16\|15 | 0 | 0 | 0 | 0 | 0 | −32.43 | 1.11 | 0.92 |
| Q96MU7-2 | YTHDC1 | 0\|0\|3 | 3 | 1 | 3\|3\|0 | 0 | 0 | 0 | 0 | 0 | −10.34 | 0.5 | 0.84 |
| O00299 | CLIC1 | 9\|8\|8 | 25 | 8.33 | 9\|7\|7 | 0 | 0 | 0 | 0 | 0 | −18.18 | 1.09 | 0.91 |
| O94925-3 | GLS | 4\|4\|0 | 8 | 2.67 | 3\|3\|6 | 0 | 0 | 0 | 0 | 0 | −19.11 | 0.67 | 0.92 |
| P31689 | DNAJA1 | 5\|4\|6 | 15 | 5 | 5\|4\|5 | 0 | 0 | 0 | 0 | 0 | −13.43 | 1.07 | 0.89 |
| P11802 | CDK4 | 3\|0\|4 | 7 | 2.33 | 3\|3\|3 | 0 | 0 | 0 | 0 | 0 | −14.75 | 0.78 | 0.87 |
| Q15046 | KARS | 3\|4\|5 | 12 | 4 | 9\|6\|8 | 0 | 0 | 0 | 0 | 0 | −26.78 | 0.52 | 0.92 |
| Q12789-3 | GTF3C1 | 3\|2\|2 | 7 | 2.33 | 3\|3\|2 | 0 | 0 | 0 | 0 | 0 | −9.29 | 0.88 | 0.87 |
| P02538 | KRT6A | 15\|14\|21 | 50 | 16.67 | 15\|15\|16 | 0 | 0 | 0 | 0 | 0 | −36.52 | 1.09 | 0.91 |
| P49915 | GMPS | 15\|14\|18 | 47 | 15.67 | 13\|15\|18 | 0 | 0 | 0 | 0 | 0 | −36.52 | 1.02 | 0.92 |
| P02533 | KRT14 | 23\|18\|22 | 63 | 21 | 25\|27\|26 | 0 | 0 | 0 | 0 | 0 | −69.41 | 0.81 | 0.92 |
| Q81WS0-5 | PHF6 | 3\|5\|5 | 13 | 4.33 | 4\|7\|5 | 0 | 0 | 0 | 0 | 0 | −17.53 | 0.81 | 0.92 |
| P04264 | KRT1 | 51\|40\|43 | 134 | 44.67 | 65\|62\|40 | 0 | 0 | 0 | 0 | 0 | −142.59 | 0.8 | 0.92 |
| Q9UJU6 | DBNL | 4\|3\|4 | 11 | 3.67 | 4\|4\|5 | 0 | 0 | 0 | 0 | 0 | −13.76 | 0.85 | 0.92 |
| Q01780 | EXOSC10 | 12\|11\|8 | 31 | 10.33 | 7\|9\|14 | 0 | 0 | 0 | 0 | 0 | −26.47 | 1.03 | 0.91 |
| O60502 | MGEAS | 7\|5\|5 | 17 | 5.67 | 6\|2\|7 | 0 | 0 | 0 | 0 | 0 | −13.07 | 1.13 | 0.89 |
| Q99426 | TBCB | 8\|11\|10 | 29 | 9.67 | 9\|8\|7 | 0 | 0 | 0 | 0 | 0 | −19.36 | 1.21 | 0.9 |
| P05198 | EIF2S1 | 13\|10\|13 | 36 | 12 | 18\|14\|15 | 0 | 0 | 0 | 0 | 0 | −44.38 | 0.77 | 0.92 |
| P07437 | TUBB | 10\|104\|12 | 336 | 112 | 15\|113\|11 | 0 | 0 | 0 | 0 | 0 | −255.26 | 0.98 | 0.92 |
| Q9Y490 | TLN1 | 7\|5\|7 | 19 | 6.33 | 6\|6\|5 | 0 | 0 | 0 | 0 | 0 | −15.5 | 1.12 | 0.9 |
| P78347-2 | GTF21 | 3\|5\|3 | 11 | 3.67 | 5\|12\|8 | 0 | 0 | 0 | 0 | 0 | −29.39 | 0.44 | 0.92 |
| P43246 | MSH2 | 13\|11\|11 | 35 | 11.67 | 10\|11\|17 | 0 | 0 | 0 | 0 | 0 | −31.54 | 0.92 | 0.92 |
| P43243 | MATR3 | 7\|8\|6 | 21 | 7 | 10\|7\|10 | 0 | 0 | 0 | 0 | 0 | −26.11 | 0.78 | 0.92 |
| P23381 | WARS | 24\|18\|19 | 61 | 20.33 | 19\|18\|18 | 0 | 0 | 0 | 0 | 0 | −41.27 | 1.11 | 0.92 |
| Q9NV17-2 | ATAD3A | 7\|6\|8 | 21 | 7 | 7\|7\|8 | 0 | 0 | 0 | 0 | 0 | −19.95 | 0.95 | 0.91 |
| P00374-2 | DHFR | 3\|3\|3 | 9 | 3 | 2\|3\|3 | 0 | 0 | 0 | 0 | 0 | −7.81 | 1.12 | 0.86 |
| P27707 | DCK | 2\|3\|2 | 7 | 2.33 | 2\|2\|2 | 0 | 0 | 0 | 0 | 0 | −6.91 | 1.17 | 0.84 |
| P27708 | CAD | 17\|19\|18 | 54 | 18 | 18\|22\|17 | 0 | 0 | 0 | 0 | 0 | −45.11 | 0.95 | 0.92 |
| Q8TEQ6 | GEMINS | 0\|0\|2 | 2 | 0.67 | 4\|2\|0 | 0 | 0 | 0 | 0 | 0 | −10.2 | 0.33 | 0.92 |
| P31946-2 | YWHAB | 7\|8\|13 | 28 | 9.33 | 8\|13\|14 | 0 | 0 | 0 | 0 | 0 | −34.53 | 0.8 | 0.91 |
| Q9Y4L1 | HYOU1 | 5\|6\|5 | 16 | 5.33 | 6\|6\|9 | 0 | 0 | 0 | 0 | 0 | −20.31 | 0.76 | 0.92 |
| Q9NQW6-1 | ANLN | 4\|5\|4 | 13 | 4.33 | 5\|4\|3 | 0 | 0 | 0 | 0 | 0 | −11.06 | 1.08 | 0.88 |
| O75600 | GCAT | 0\|0\|2 | 2 | 0.67 | 0\|2\|2 | 0 | 0.01 | 0 | 0.01 | 0 | −7.8 | 0.5 | 0.82 |
| P23258 | TUBG1 | 6\|7\|4 | 17 | 5.67 | 6\|4\|5 | 0 | 0 | 0 | 0 | 0 | −14.57 | 1.13 | 0.89 |
| Q81W19-3 | IMGA | 0\|2\|2 | 4 | 1.33 | 10\|17\|13 | 0 | 0 | 0 | 0 | 0 | −60.53 | 0.1 | 0.92 |
| Q14103-3 | HNRNPD | 21\|20\|21 | 62 | 20.67 | 24\|18\|20 | 0 | 0 | 0 | 0 | 0 | −46.6 | 1 | 0.92 |
| P20042 | EIF2S2 | 13\|11\|16 | 40 | 13.33 | 15\|14\|10 | 0 | 0 | 0 | 0 | 0 | −32.69 | 1.03 | 0.91 |
| Q9Y281-3 | CFL2 | 7\|7\|5 | 19 | 6.33 | 5\|6\|8 | 0 | 0 | 0 | 0 | 0 | −17.88 | 1 | 0.9 |
| P49207 | RPL34 | 3\|3\|4 | 10 | 3.33 | 5\|4\|3 | 0 | 0 | 0 | 0 | 0 | −12.5 | 0.83 | 0.92 |
| P41240 | CSK | 3\|3\|3 | 9 | 3 | 3\|3\|0 | 0 | 0 | 0 | 0 | 0 | −5.36 | 1.5 | 0.81 |
| Q9GZZ1 | NAASO | 5\|3\|6 | 14 | 4.67 | 4\|4\|5 | 0 | 0 | 0 | 0 | 0 | −13.76 | 1.08 | 0.88 |
| O94966-2 | USP19 | 3\|2\|3 | 8 | 2.67 | 4\|2\|0 | 0 | 0.01 | 0 | 0.01 | 0 | −6.67 | 1.33 | 0.82 |
| P25705 | ATPSA1 | 36\|26\|35 | 97 | 32.33 | 35\|37\|41 | 0 | 0 | 0 | 0 | 0 | −99.54 | 0.86 | 0.92 |
| O43615 | TIMM44 | 8\|6\|9 | 23 | 7.67 | 7\|6\|6 | 0 | 0 | 0 | 0 | 0 | −16.39 | 1.21 | 0.9 |
| Q8WWM7 | ATXN2L | 9\|8\|8 | 25 | 8.33 | 6\|5\|8 | 0 | 0 | 0 | 0 | 0 | −13.5 | 1.32 | 0.9 |
| P61313 | RPL15 | 9\|8\|8 | 25 | 8.33 | 13\|10\|10 | 0 | 0 | 0 | 0 | 0 | −30.19 | 0.76 | 0.92 |
| Q9HCM7 | FBRSL1 | 4\|4\|4 | 12 | 4 | 2\|4\|4 | 0 | 0 | 0 | 0 | 0 | −8.71 | 1.2 | 0.87 |
| P11498 | PC | 50\|845\|80 | 2501 | 833.67 | 56\|751\|76 | 0 | 0 | 0 | 0 | 0 | #NAME? | 1.1 | 0.92 |
| Q13263 | TRIM28 | 8\|7\|5 | 20 | 6.67 | 8\|8\|12 | 0 | 0 | 0 | 0 | 0 | −29.27 | 0.71 | 0.92 |
| Q16822 | PCK2 | 2\|0\|2 | 4 | 1.33 | 4\|4\|4 | 0 | 0 | 0 | 0 | 0 | −19.11 | 0.33 | 0.92 |
| O75131 | CPNE3 | 6\|6\|7 | 19 | 6.33 | 9\|5\|4 | 0 | 0 | 0 | 0 | 0 | −15.24 | 1.06 | 0.9 |
| P61006 | RAB8A | 9\|9\|10 | 28 | 9.33 | 8\|7\|7 | 0 | 0 | 0 | 0 | 0 | −15.57 | 1.27 | 0.9 |
| P53621 | COPA | 17\|18\|13 | 48 | 16 | 24\|16\|21 | 0 | 0 | 0 | 0 | 0 | −56.79 | 0.79 | 0.92 |
| O95453-4 | PARN | 2\|0\|0 | 2 | 0.67 | 2\|2\|2 | 0 | 0 | 0 | 0 | 0 | −10.44 | 0.33 | 0.92 |
| P22626 | HNRNPA281 | 4\|2\|4 | 10 | 3.33 | 2\|5\|5 | 0 | 0 | 0 | 0 | 0 | −14.21 | 0.83 | 0.92 |
| Q6YN16-2 | HSDL2 | 3\|4\|3 | 10 | 3.33 | 3\|0\|4 | 0 | 0.01 | 0 | 0.01 | 0 | −6.41 | 1.43 | 0.82 |
| Q15029-2 | EFTUD2 | 12\|9\|10 | 31 | 10.33 | 22\|19\|22 | 0 | 0 | 0 | 0 | 0 | −67.49 | 0.49 | 0.92 |
| Q7L2E3-3 | DHX30 | 13\|9\|10 | 32 | 10.67 | 13\|8\|10 | 0 | 0 | 0 | 0 | 0 | −26.16 | 1.03 | 0.91 |
| P54136 | RARS | 16\|13\|12 | 41 | 13.67 | 13\|13\|13 | 0 | 0 | 0 | 0 | 0 | −31.17 | 1.05 | 0.91 |
| Q02952-3 | AKAP12 | 5\|7\|7 | 19 | 6.33 | 4\|4\|6 | 0 | 0 | 0 | 0 | 0 | −11.96 | 1.36 | 0.88 |
| Q96PV6 | LENG8 | 2\|2\|3 | 7 | 2.33 | 2\|2\|2 | 0 | 0 | 0 | 0 | 0 | −6.91 | 1.17 | 0.84 |
| Q06830 | PRDX1 | 9\|8\|8 | 25 | 8.33 | 6\|5\|6 | 0 | 0 | 0 | 0 | 0 | −11.18 | 1.47 | 0.88 |
| O00116 | AGPS | 4\|6\|4 | 14 | 4.67 | 6\|5\|7 | 0 | 0 | 0 | 0 | 0 | −18.27 | 0.78 | 0.92 |
| Q03519-2 | TAP2 | 3\|0\|3 | 6 | 2 | 4\|2\|0 | 0 | 0.01 | 0 | 0.01 | 0 | −10.2 | 1 | 0.82 |
| P30520 | ADSS | 5\|5\|7 | 17 | 5.67 | 8\|6\|8 | 0 | 0 | 0 | 0 | 0 | −21.58 | 0.77 | 0.92 |
| Q15428 | SF3A2 | 4\|4\|6 | 14 | 4.67 | 3\|5\|10 | 0 | 0 | 0 | 0 | 0 | −17.98 | 0.78 | 0.92 |
| P10155-3 | TROVE2 | 3\|3\|2 | 8 | 2.67 | 2\|3\|2 | 0 | 0 | 0 | 0 | 0 | −8.1 | 1.14 | 0.85 |
| P49588 | AARS | 8\|6\|6 | 20 | 6.67 | 11\|10\|11 | 0 | 0 | 0 | 0 | 0 | −32.54 | 0.63 | 0.92 |
| Q9HCC0 | MCCC2 | 27\|34\|29 | 90 | 30 | 29\|26\|25 | 0 | 0 | 0 | 0 | 0 | −57.59 | 1.12 | 0.92 |
| P30419 | NMT1 | 3\|3\|7 | 13 | 4.33 | 5\|2\|4 | 0 | 0 | 0 | 0 | 0 | −11.36 | 1.18 | 0.85 |
| Q14498-2 | RBM39 | 7\|4\|6 | 17 | 5.67 | 7\|4\|9 | 0 | 0 | 0 | 0 | 0 | −20.83 | 0.85 | 0.91 |
| P60842 | EIF4A1 | 60\|59\|55 | 174 | 58 | 64\|70\|69 | 0 | 0 | 0 | 0 | 0 | −162.55 | 0.86 | 0.92 |
| P47755 | CAPZA2 | 4\|5\|5 | 14 | 4.67 | 4\|5\|6 | 0 | 0 | 0 | 0 | 0 | −14.57 | 0.93 | 0.92 |
| Q8TDD1 | DDX54 | 5\|3\|3 | 11 | 3.67 | 2\|5\|3 | 0 | 0 | 0 | 0 | 0 | −10.17 | 1.1 | 0.87 |
| P48507 | GCLM | 5\|5\|5 | 15 | 5 | 4\|2\|3 | 0 | 0 | 0 | 0 | 0 | −6.14 | 1.67 | 0.83 |
| P06733 | ENO1 | 87\|83\|96 | 266 | 88.67 | 85\|94\|105 | 0 | 0 | 0 | 0 | 0 | −216.72 | 0.94 | 0.92 |
| Q9BRX2 | PELO | 6\|7\|6 | 19 | 6.33 | 6\|5\|7 | 0 | 0 | 0 | 0 | 0 | −15.21 | 1.06 | 0.9 |
| Q96D46 | INMD3 | 5\|6\|4 | 15 | 5 | 6\|6\|6 | 0 | 0 | 0 | 0 | 0 | −18.27 | 0.83 | 0.92 |
| O15160 | POLRIC | 5\|6\|7 | 18 | 6 | 4\|5\|6 | 0 | 0 | 0 | 0 | 0 | −13.14 | 1.2 | 0.89 |
| P51659 | HSD1784 | 3\|5\|5 | 13 | 4.33 | 7\|7\|8 | 0 | 0 | 0 | 0 | 0 | −25.42 | 0.59 | 0.92 |
| P60900-2 | PSMA6 | 4\|3\|3 | 10 | 3.33 | 4\|4\|2 | 0 | 0 | 0 | 0 | 0 | −10.17 | 1 | 0.88 |
| P56192 | MARS | 6\|7\|7 | 20 | 6.67 | 7\|8\|15 | 0 | 0 | 0 | 0 | 0 | −29.72 | 0.67 | 0.92 |
| P35573-2 | AGL | 0\|2\|0 | 2 | 0.67 | 2\|2\|0 | 0 | 0.01 | 0 | 0.01 | 0 | −7.8 | 0.5 | 0.82 |
| P20700 | LMNB1 | 22\|21\|20 | 63 | 21 | 17\|21\|19 | 0 | 0 | 0 | 0 | 0 | −40.72 | 1.11 | 0.92 |
| P55884 | EIF3B | 3\|3\|3 | 9 | 3 | 2\|3\|2 | 0 | 0 | 0 | 0 | 0 | −6.63 | 1.29 | 0.84 |
| Q01650 | SLC7A5 | 6\|5\|5 | 16 | 5.33 | 8\|5\|4 | 0 | 0 | 0 | 0 | 0 | −15.5 | 0.94 | 0.9 |
| P07737 | PFN1 | 48\|45\|45 | 138 | 46 | 48\|47\|45 | 0 | 0 | 0 | 0 | 0 | −101.98 | 0.99 | 0.92 |
| P08779 | KRT16 | 15\|12\|18 | 45 | 15 | 15\|23\|19 | 0 | 0 | 0 | 0 | 0 | −53.5 | 0.79 | 0.92 |
| P52294 | KPNA1 | 0\|3\|3 | 6 | 2 | 3\|2\|4 | 0 | 0 | 0 | 0 | 0 | −14.75 | 0.67 | 0.92 |
| P52292 | KPNA2 | 5\|7\|5 | 17 | 5.67 | 4\|0\|7 | 0 | 0 | 0 | 0 | 0 | −7.94 | 1.55 | 0.84 |
| P00966 | ASS1 | 33\|33\|34 | 100 | 33.33 | 30\|41\|39 | 0 | 0 | 0 | 0 | 0 | −84.25 | 0.91 | 0.92 |
| P62910 | RPL32 | 8\|6\|7 | 21 | 7 | 7\|5\|6 | 0 | 0 | 0 | 0 | 0 | −15.21 | 1.17 | 0.9 |
| P14618-2 | PKM | 69\|60\|67 | 196 | 65.33 | 72\|62\|72 | 0 | 0 | 0 | 0 | 0 | −158.2 | 0.95 | 0.92 |
| Q03518 | TAP1 | 4\|3\|2 | 9 | 3 | 3\|4\|3 | 0 | 0 | 0 | 0 | 0 | −11.71 | 0.9 | 0.88 |
| P62917 | RPL8 | 12\|8\|11 | 31 | 10.33 | 10\|6\|9 | 0 | 0 | 0 | 0 | 0 | −20.53 | 1.24 | 0.9 |
| O00425 | IGF2BP3 | 7\|6\|8 | 21 | 7 | 11\|10\|10 | 0 | 0 | 0 | 0 | 0 | −31.24 | 0.68 | 0.92 |
| Q10471 | GALNT2 | 3\|4\|4 | 11 | 3.67 | 5\|3\|4 | 0 | 0 | 0 | 0 | 0 | −12.5 | 0.92 | 0.92 |
| Q00796 | SORD | 5\|4\|5 | 14 | 4.67 | 3\|4\|3 | 0 | 0 | 0 | 0 | 0 | −8.71 | 1.4 | 0.86 |
| Q9H0U4 | RAB18 | 16\|18\|18 | 52 | 17.33 | 16\|19\|18 | 0 | 0 | 0 | 0 | 0 | −41.86 | 0.98 | 0.92 |
| O00743-2 | PPP6C | 3\|3\|2 | 8 | 2.67 | 2\|3\|3 | 0 | 0 | 0 | 0 | 0 | −9.29 | 1 | 0.86 |
| P23396 | RPS3 | 28\|26\|29 | 83 | 27.67 | 32\|35\|35 | 0 | 0 | 0 | 0 | 0 | −85.71 | 0.81 | 0.92 |
| P35658-2 | INUP214 | 26\|22\|26 | 74 | 24.67 | 22\|26\|25 | 0 | 0 | 0 | 0 | 0 | −56.67 | 1.01 | 0.92 |
| P35268 | RPL22 | 5\|7\|8 | 20 | 6.67 | 7\|7\|7 | 0 | 0 | 0 | 0 | 0 | −20.31 | 0.95 | 0.91 |
| Q71U36-2 | TUBA1A | 81\|71\|75 | 227 | 75.67 | 77\|72\|76 | 0 | 0 | 0 | 0 | 0 | −164.18 | 1.01 | 0.92 |
| Q10713 | PMPCA | 4\|3\|3 | 10 | 3.33 | 3\|5\|4 | 0 | 0 | 0 | 0 | 0 | −12.5 | 0.83 | 0.92 |
| P00367 | GLUD1 | 0\|3\|3 | 6 | 2 | 2\|3\|3 | 0 | 0 | 0 | 0 | 0 | −13.39 | 0.75 | 0.86 |
| Q9UKN8 | GTF3C4 | 5\|5\|5 | 15 | 5 | 4\|5\|7 | 0 | 0 | 0 | 0 | 0 | −14.32 | 0.94 | 0.92 |
| P00568 | AK1 | 3\|2\|0 | 5 | 1.67 | 2\|2\|3 | 0 | 0 | 0 | 0 | 0 | −11.93 | 0.71 | 0.86 |
| Q3KQU3-4 | MAP7D1 | 7\|5\|5 | 17 | 5.67 | 8\|7\|9 | 0 | 0 | 0 | 0 | 0 | −24.09 | 0.71 | 0.92 |
| Q92499 | DDX1 | 13\|11\|9 | 33 | 11 | 6\|12\|10 | 0 | 0 | 0 | 0 | 0 | −22.61 | 1.18 | 0.91 |
| P11940-2 | PABPC1 | 5\|2\|6 | 13 | 4.33 | 4\|6\|5 | 0 | 0 | 0 | 0 | 0 | −18.16 | 0.87 | 0.89 |
| P28288 | ABCD3 | 4\|4\|8 | 16 | 5.33 | 4\|6\|6 | 0 | 0 | 0 | 0 | 0 | −15.82 | 1 | 0.89 |
| P23246 | SFPQ | 27\|28\|24 | 79 | 26.33 | 26\|23\|27 | 0 | 0 | 0 | 0 | 0 | −57.25 | 1.04 | 0.92 |
| Q7KZF4 | SND1 | 34\|30\|37 | 101 | 33.67 | 33\|32\|37 | 0 | 0 | 0 | 0 | 0 | −79.24 | 0.99 | 0.92 |
| P24752 | ACAT1 | 14\|12\|16 | 42 | 14 | 14\|12\|10 | 0 | 0 | 0 | 0 | 0 | −27.65 | 1.17 | 0.91 |
| Q99714 | HSD17810 | 2\|0\|3 | 5 | 1.67 | 2\|2\|2 | 0 | 0 | 0 | 0 | 0 | −10.44 | 0.83 | 0.84 |
| Q9H307 | PNN | 3\|0\|2 | 5 | 1.67 | 3\|6\|3 | 0 | 0 | 0 | 0 | 0 | −19.11 | 0.42 | 0.92 |
| Q14157-1 | UBAP2L | 11\|8\|15 | 34 | 11.33 | 17\|6\|14 | 0 | 0 | 0 | 0 | 0 | −34.88 | 0.92 | 0.91 |
| P52701 | MSH6 | 5\|5\|9 | 19 | 6.33 | 7\|4\|4 | 0 | 0 | 0 | 0 | 0 | −13.14 | 1.27 | 0.87 |
| Q5T9A4 | ATAD3B | 7\|5\|5 | 17 | 5.67 | 5\|5\|5 | 0 | 0 | 0 | 0 | 0 | −13.14 | 1.13 | 0.89 |
| P43686-2 | PSMC4 | 7\|6\|8 | 21 | 7 | 5\|7\|6 | 0 | 0 | 0 | 0 | 0 | −15.21 | 1.17 | 0.9 |
| Q14166 | TTLL12 | 7\|8\|7 | 22 | 7.33 | 8\|5\|9 | 0 | 0 | 0 | 0 | 0 | −18.46 | 1 | 0.91 |
| Q14160 | SCRIB | 0\|0\|2 | 2 | 0.67 | 3\|5\|0 | 0 | 0 | 0 | 0 | 0 | −13.16 | 0.25 | 0.92 |
| P10809 | HSPD1 | 24\|21\|23 | 68 | 22.67 | 24\|19\|27 | 0 | 0 | 0 | 0 | 0 | −54.6 | 0.97 | 0.92 |
| Q7Z6E9-2 | RBBP6 | 2\|3\|4 | 9 | 3 | 10\|10\|12 | 0 | 0 | 0 | 0 | 0 | −41.8 | 0.28 | 0.92 |
| Q08499-6 | PDE4D | 0\|0\|2 | 2 | 0.67 | 2\|0\|2 | 0 | 0.01 | 0 | 0.01 | 0 | −7.8 | 0.5 | 0.82 |
| Q16836 | HADH | 4\|3\|4 | 11 | 3.67 | 6\|6\|3 | 0 | 0 | 0 | 0 | 0 | −16.23 | 0.73 | 0.92 |
| Q9UJU6-2 | DBNL | 4\|3\|4 | 11 | 3.67 | 4\|4\|5 | 0 | 0 | 0 | 0 | 0 | −13.76 | 0.85 | 0.92 |
| Q16543 | CDC37 | 5\|4\|7 | 16 | 5.33 | 5\|6\|4 | 0 | 0 | 0 | 0 | 0 | −14.57 | 1.07 | 0.89 |
| Q13257 | MAD2L1 | 3\|2\|2 | 7 | 2.33 | 3\|2\|4 | 0 | 0 | 0 | 0 | 0 | −10.42 | 0.78 | 0.92 |
| P49368 | CCT3 | 33\|31\|38 | 102 | 34 | 36\|36\|41 | 0 | 0 | 0 | 0 | 0 | −91.04 | 0.9 | 0.92 |
| P32119 | PRDX2 | 5\|5\|4 | 14 | 4.67 | 6\|5\|3 | 0 | 0 | 0 | 0 | 0 | −13.43 | 1 | 0.89 |
| O07866-8 | KLC1 | 5\|4\|3 | 12 | 4 | 3\|4\|2 | 0 | 0 | 0 | 0 | 0 | −8.99 | 1.33 | 0.85 |
| P19013 | KRT4 | 3\|3\|4 | 10 | 3.33 | 4\|2\|3 | 0 | 0 | 0 | 0 | 0 | −8.99 | 1.11 | 0.87 |
| P26196 | DDX6 | 6\|6\|3 | 15 | 5 | 4\|5\|4 | 0 | 0 | 0 | 0 | 0 | −13.76 | 1.15 | 0.88 |
| P61073 | CXCR4 | 2\|2\|2 | 6 | 2 | 3\|2\|2 | 0 | 0 | 0 | 0 | 0 | −8.1 | 0.86 | 0.92 |
| Q9BUQ8 | DDX23 | 3\|4\|5 | 12 | 4 | 8\|2\|7 | 0 | 0 | 0 | 0 | 0 | −18.67 | 0.71 | 0.92 |
| Q9BWE0 | REPIN1 | 5\|5\|2 | 12 | 4 | 5\|3\|4 | 0 | 0 | 0 | 0 | 0 | −14.21 | 1 | 0.88 |
| Q9BWD1 | ACAT2 | 12\|9\|11 | 32 | 10.67 | 13\|10\|11 | 0 | 0 | 0 | 0 | 0 | −29.79 | 0.94 | 0.91 |
| Q9Y5M8 | SRPRB | 5\|4\|6 | 15 | 5 | 6\|5\|4 | 0 | 0 | 0 | 0 | 0 | −14.57 | 1 | 0.89 |
| P33992 | MCM5 | 18\|16\|15 | 49 | 16.33 | 18\|15\|20 | 0 | 0 | 0 | 0 | 0 | −43.4 | 0.92 | 0.92 |
| P33993 | MCM7 | 29\|25\|29 | 83 | 27.67 | 29\|32\|31 | 0 | 0 | 0 | 0 | 0 | −75.01 | 0.9 | 0.92 |
| P33991 | MCM4 | 18\|16\|19 | 53 | 17.67 | 19\|21\|12 | 0 | 0 | 0 | 0 | 0 | −40.64 | 1.02 | 0.92 |
| P50914 | RPL14 | 3\|0\|0 | 3 | 3 | 3\|5\|5 | 0 | 0 | 0 | 0 | 0 | −20.61 | 0.23 | 0.92 |
| P49755 | TMED10 | 2\|3\|2 | 7 | 2.33 | 3\|4\|6 | 0 | 0 | 0 | 0 | 0 | −15.55 | 0.54 | 0.92 |
| P49756 | RBM25 | 2\|3\|0 | 5 | 1.67 | 4\|5\|4 | 0 | 0 | 0 | 0 | 0 | −20.61 | 0.38 | 0.92 |
| P61106 | RAB14 | 12\|19\|15 | 46 | 15.33 | 14\|16\|18 | 0 | 0 | 0 | 0 | 0 | −42.12 | 0.96 | 0.92 |
| O43242 | PSMD3 | 13\|14\|10 | 37 | 12.33 | 9\|15\|14 | 0 | 0 | 0 | 0 | 0 | −33.08 | 0.97 | 0.92 |
| P49591 | SARS | 3\|4\|2 | 9 | 3 | 5\|4\|6 | 0 | 0 | 0 | 0 | 0 | −18.16 | 0.6 | 0.92 |
| Q86V48-2 | LUZP1 | 3\|2\|0 | 5 | 1.67 | 4\|4\|7 | 0 | 0 | 0 | 0 | 0 | −23.5 | 0.33 | 0.92 |
| P61586 | RHOA | 3\|2\|5 | 10 | 3.33 | 4\|4\|5 | 0 | 0 | 0 | 0 | 0 | −15.55 | 0.77 | 0.89 |
| Q9H0D6 | XRN2 | 5\|6\|6 | 17 | 5.67 | 5\|7\|5 | 0 | 0 | 0 | 0 | 0 | −15.5 | 1 | 0.9 |
| O43172-2 | PRPF4 | 10\|7\|8 | 25 | 8.33 | 9\|8\|10 | 0 | 0 | 0 | 0 | 0 | −24.43 | 0.93 | 0.91 |
| Q72417 | NUFIP2 | 7\|6\|6 | 19 | 6.33 | 4\|8\|5 | 0 | 0 | 0 | 0 | 0 | −14.03 | 1.12 | 0.9 |
| P16401 | HIST1H1B | 12\|4\|6 | 22 | 7.33 | 8\|6\|5 | 0 | 0 | 0 | 0 | 0 | −19.55 | 1.16 | 0.87 |
| Q9UBX3 | SLC25A10 | 6\|7\|5 | 18 | 6 | 2\|5\|4 | 0 | 0 | 0 | 0 | 0 | −8.45 | 1.64 | 0.84 |
| Q9HC07 | TMEM165 | 3\|2\|2 | 7 | 2.33 | 2\|3\|3 | 0 | 0 | 0 | 0 | 0 | −9.29 | 0.88 | 0.87 |
| Q9NR12 | PDLIM7 | 12\|12\|11 | 35 | 11.67 | 13\|12\|10 | 0 | 0 | 0 | 0 | 0 | −27.93 | 1 | 0.91 |
| P28838-2 | LAP3 | 6\|7\|7 | 20 | 6.67 | 5\|6\|6 | 0 | 0 | 0 | 0 | 0 | −14.03 | 1.18 | 0.9 |
| Q8WVV9-5 | HNRNPLL | 3\|0\|2 | 5 | 1.67 | 3\|2\|3 | 0 | 0 | 0 | 0 | 0 | −13.39 | 0.63 | 0.87 |
| P35613-2 | BSG | 6\|6\|6 | 18 | 6 | 5\|7\|5 | 0 | 0 | 0 | 0 | 0 | −14.03 | 1.06 | 0.9 |
| Q9NP92 | MRPS30 | 2\|2\|0 | 4 | 1.33 | 0\|2\|3 | 0 | 0 | 0 | 0 | 0 | −9.13 | 0.8 | 0.83 |
| Q86X55-1 | CARM1 | 6\|6\|5 | 17 | 5.67 | 5\|5\|2 | 0 | 0 | 0 | 0 | 0 | −9.61 | 1.42 | 0.87 |
| O95782-2 | AP2A1 | 6\|5\|6 | 17 | 5.67 | 6\|5\|7 | 0 | 0 | 0 | 0 | 0 | −16.65 | 0.94 | 0.92 |
| O94979-6 | SEC31A | 8\|6\|9 | 23 | 7.67 | 4\|12\|9 | 0 | 0 | 0 | 0 | 0 | −23.4 | 0.92 | 0.91 |
| Q96HN2-2 | AHCYL2 | 6\|5\|4 | 15 | 5 | 7\|6\|5 | 0 | 0 | 0 | 0 | 0 | −18.27 | 0.83 | 0.92 |
| Q96IU4 | ABHD14B | 3\|3\|2 | 8 | 2.67 | 2\|3\|2 | 0 | 0 | 0 | 0 | 0 | −8.1 | 1.14 | 0.85 |
| Q9BW92 | TARS2 | 4\|4\|3 | 11 | 3.67 | 3\|2\|3 | 0 | 0 | 0 | 0 | 0 | −7.81 | 1.38 | 0.84 |
| P27824 | CANX | 10\|8\|9 | 27 | 9 | 6\|6\|8 | 0 | 0 | 0 | 0 | 0 | −14.67 | 1.35 | 0.89 |
| P40763-3 | STAT3 | 15\|10\|13 | 38 | 12.67 | 14\|10\|14 | 0 | 0 | 0 | 0 | 0 | −33.08 | 3 | 0.91 |
| P62906 | RPL10A | 7\|9\|8 | 24 | 8 | 10\|11\|11 | 0 | 0 | 0 | 0 | 0 | −30.69 | 0.75 | 0.92 |
| P83731 | RPL24 | 7\|7\|8 | 22 | 7.33 | 6\|5\|7 | 0 | 0 | 0 | 0 | 0 | −13.76 | 1.22 | 0.9 |
| P40616-2 | ARL0 | 2\|2\|2 | 6 | 2 | 2\|5\|5 | 0 | 0 | 0 | 0 | 0 | −14.21 | 0.5 | 0.92 |
| P28070 | PSMB4 | 5\|5\|3 | 13 | 4.33 | 4\|3\|4 | 0 | 0 | 0 | 0 | 0 | −11.36 | 1.18 | 0.87 |
| P28072 | PSMB6 | 2\|3\|2 | 7 | 2.33 | 3\|3\|2 | 0 | 0 | 0 | 0 | 0 | −9.29 | 0.88 | 0.87 |
| Q15393 | SF3B3 | 6\|5\|6 | 17 | 5.67 | 7\|4\|2 | 0 | 0 | 0 | 0 | 0 | −10.69 | 1.31 | 0.88 |
| Q9Y2W1 | THRAP3 | 4\|6\|8 | 18 | 6 | 6\|9\|8 | 0 | 0 | 0 | 0 | 0 | −24.7 | 0.78 | 0.91 |
| O60563 | CCNT1 | 4\|6\|3 | 13 | 4.33 | 5\|4\|6 | 0 | 0 | 0 | 0 | 0 | −16.23 | 0.87 | 0.89 |
| Q9UMR2 | DDX19B | 11\|7\|7 | 25 | 8.33 | 7\|2\|7 | 0 | 0 | 0 | 0 | 0 | −11.32 | 1.56 | 0.85 |
| P35908 | KRT2 | 48\|31\|34 | 113 | 37.67 | 40\|51\|33 | 0 | 0 | 0 | 0 | 0 | −104.43 | 0.91 | 0.92 |
| Q81Y37 | DHX37 | 2\|2\|0 | 4 | 1.33 | 2\|2\|2 | 0 | 0 | 0 | 0 | 0 | −10.44 | 0.67 | 0.92 |
| P53004 | BLVRA | 5\|2\|3 | 10 | 3.33 | 4\|2\|3 | 0 | 0 | 0 | 0 | 0 | −10.42 | 1.11 | 0.85 |
| Q8N6H7 | ARFGAP2 | 5\|6\|7 | 18 | 6 | 5\|4\|3 | 0 | 0 | 0 | 0 | 0 | −9.61 | 1.5 | 0.86 |
| Q9Y305-3 | ACOT9 | 7\|6\|5 | 18 | 6 | 5\|7\|3 | 0 | 0 | 0 | 0 | 0 | −13.14 | 1.2 | 0.89 |
| P00558 | PGK1 | 27\|33\|31 | 91 | 30.33 | 31\|29\|28 | 0 | 0 | 0 | 0 | 0 | −67.03 | 1.03 | 0.92 |
| Q9Y220-2 | SUGT1 | 2\|2\|2 | 6 | 2 | 2\|3\|4 | 0 | 0 | 0 | 0 | 0 | −10.42 | 0.67 | 0.92 |
| Q14137-2 | BOP1 | 3\|2\|4 | 9 | 3 | 0\|4\|4 | 0 | 0 | 0 | 0 | 0 | −9.13 | 1.12 | 0.85 |
| Q14258 | TRIM25 | 9\|7\|7 | 23 | 7.67 | 8\|8\|9 | 0 | 0 | 0 | 0 | 0 | −22.02 | 0.92 | 0.91 |
| Q9BY77-2 | POLDIP3 | 2\|0\|2 | 4 | 1.33 | 3\|2\|3 | 0 | 0 | 0 | 0 | 0 | −13.39 | 0.5 | 0.92 |
| Q05048 | CSTF1 | 4\|5\|5 | 14 | 4.67 | 4\|5\|4 | 0 | 0 | 0 | 0 | 0 | −12.24 | 1.08 | 0.89 |
| Q5VT06 | CEP350 | 19\|17\|22 | 58 | 19.33 | 35\|43\|36 | 0 | 0 | 0 | 0 | 0 | −118.64 | 0.51 | 0.92 |
| P09382 | LGALS1 | 3\|2\|3 | 8 | 2.67 | 3\|3\|4 | 0 | 0 | 0 | 0 | 0 | −11.71 | 0.8 | 0.92 |
| P53396 | ACLY | 35\|34\|35 | 104 | 34.67 | 44\|42\|45 | 0 | | 0 | 0 | 0 | −108.31 | 0.79 | 0.92 |
| Q99700-2 | ATXN2 | 6\|2\|4 | 12 | 4 | 4\|3\|3 | 0 | 0 | 0 | 0 | 0 | −11.71 | 1.2 | 0.85 |
| P41227 | NAA10 | 6\|4\|4 | 14 | 4.67 | 5\|3\|6 | 0 | 0 | 0 | 0 | 0 | −13.43 | 1 | 0.89 |
| P29353-3 | SHC3 | 9\|8\|8 | 25 | 8.33 | 6\|6\|8 | 0 | 0 | 0 | 0 | 0 | −14.67 | 1.25 | 0.9 |
| O75717 | WDHD1 | 2\|0\|2 | 4 | 1.33 | 0\|3\|5 | 0 | 0 | 0 | 0 | 0 | −13.16 | 0.5 | 0.92 |
| P47897-2 | QARS | 3\|3\|0 | 6 | 2 | 4\|5\|0 | 0 | 0 | 0 | 0 | 0 | −14.43 | 0.67 | 0.92 |
| Q81WVR0 | ZC3H7A | 4\|5\|2 | 11 | 3.67 | 3\|4\|3 | 0 | 0 | 0 | 0 | 0 | −11.71 | 1.1 | 0.86 |
| Q02809 | PLOD1 | 11\|10\|10 | 31 | 10.33 | 11\|8\|7 | 0 | 0 | 0 | 0 | 0 | −18.81 | 1.19 | 0.91 |
| P06899 | HIST1H2BJ | 4\|5\|5 | 14 | 4.67 | 6\|9\|6 | 0 | 0 | 0 | 0 | 0 | −22.08 | 0.67 | 0.92 |
| P07384 | CAPN1 | 3\|2\|0 | 5 | 1.67 | 4\|5\|0 | 0 | 0 | 0 | 0 | 0 | −14.43 | 0.56 | 0.92 |
| Q9NZ01 | TECR | 6\|5\|5 | 16 | 5.33 | 5\|5\|5 | 0 | 0 | 0 | 0 | 0 | −13.14 | 1.07 | 0.89 |
| Q16531 | DDB1 | 8\|4\|4 | 16 | 5.33 | 3\|5\|8 | 0 | 0 | 0 | 0 | 0 | −15.83 | 1 | 0.89 |
| P06576 | ATPSB | 7\|5\|8 | 20 | 6.67 | 9\|9\|11 | 0 | 0 | 0 | 0 | 0 | −30.58 | 0.69 | 0.92 |
| O75150 | RNF40 | 7\|7\|5 | 19 | 6.33 | 4\|4\|5 | 0 | 0 | 0 | 0 | 0 | −10.78 | 1.46 | 0.87 |
| O75153 | CLUH | 5\|3\|6 | 14 | 4.67 | 8\|4\|5 | 0 | 0 | 0 | 0 | 0 | −18.83 | 0.82 | 0.9 |
| O75152 | ZC3H11A | 2\|3\|0 | 5 | 1.67 | 2\|3\|3 | 0 | 0 | 0 | 0 | 0 | −13.39 | 0.63 | 0.87 |
| Q02218-2 | OGDH | 2\|0\|2 | 4 | 1.33 | 2\|4\|5 | 0 | 0 | 0 | 0 | 0 | −17.71 | 0.36 | 0.92 |
| Q99707 | MTR | 4\|2\|2 | 8 | 2.67 | 5\|6\|4 | 0 | 0 | 0 | 0 | 0 | −18.16 | 0.53 | 0.92 |
| Q15717 | ELAVLI | 8\|9\|9 | 26 | 8.67 | 10\|8\|9 | 0 | 0 | 0 | 0 | 0 | −22.87 | 0.96 | 0.92 |
| P07954-2 | FH | 3\|3\|3 | 9 | 3 | 2\|4\|4 | 0 | 0 | 0 | 0 | 0 | −10.17 | 0.9 | 0.92 |
| O60749-2 | SNX2 | 2\|0\|0 | 2 | 0.67 | 2\|4\|3 | 0 | 0 | 0 | 0 | 0 | −14.75 | 0.22 | 0.92 |
| O43432-4 | EIF4G3 | 4\|5\|4 | 13 | 4.33 | 2\|7\|4 | 0 | 0 | 0 | 0 | 0 | −12.17 | 1 | 0.89 |
| Q07157-2 | TIP1 | 3\|2\|3 | 8 | 2.67 | 0\|3\|3 | 0 | 0 | 0 | 0 | 0 | −6.82 | 1.33 | 0.82 |
| Q96DG6 | CMBL | 0\|2\|0 | 2 | 0.67 | 2\|2\|2 | 0 | 0 | 0 | 0 | 0 | −10.44 | 0.33 | 0.92 |
| P36404-2 | ARL2 | 2\|2\|4 | 8 | 2.67 | 2\|3\|3 | 0 | 0 | 0 | 0 | 0 | −9.29 | 3 | 0.85 |
| Q96FW1 | OTUB1 | 7\|6\|6 | 19 | 6.33 | 6\|8\|8 | 0 | 0 | 0 | 0 | 0 | −19.95 | 0.86 | 0.92 |
| P36578 | RPL4 | 47\|36\|39 | 122 | 40.67 | 39\|40\|44 | 0 | 0 | 0 | 0 | 0 | −95.27 | 0.99 | 0.92 |
| P63173 | RPL38 | 12\|10\|12 | 34 | 11.33 | 13\|10\|9 | 0 | 0 | 0 | 0 | 0 | −25.86 | 1.06 | 0.91 |
| Q8N1G0-2 | ZNF687 | 0\|0\|2 | 2 | 0.67 | 3\|3\|3 | 0 | 0 | 0 | 0 | 0 | −14.75 | 0.22 | 0.92 |
| P30508 | HLA-C | 6\|5\|7 | 18 | 6 | 6\|6\|4 | 0 | 0 | 0 | 0 | 0 | −14.32 | 1.12 | 0.89 |
| Q9Y3Y2-4 | CHTOP | 12\|11\|10 | 33 | 11 | 9\|10\|8 | 0 | 0 | 0 | 0 | 0 | −19.98 | 1.22 | 0.91 |
| Q9BY44 | EIF2A | 13\|10\|12 | 35 | 11.67 | 9\|5\|9 | 0 | 0 | 0 | 0 | 0 | −15.32 | 1.52 | 0.89 |
| P12004 | PCNA | 9\|9\|12 | 30 | 10 | 9\|11\|16 | 0 | 0 | 0 | 0 | 0 | −32.31 | 0.83 | 0.92 |
| Q9BQ67 | GRWD1 | 4\|4\|5 | 13 | 4.33 | 5\|4\|6 | 0 | 0 | 0 | 0 | 0 | −14.57 | 0.87 | 0.92 |
| P07951-3 | PM2 | 2\|0\|3 | 5 | 1.67 | 2\|2\|2 | 0 | 0 | 0 | 0 | 0 | −10.44 | 0.83 | 0.84 |
| P22314-2 | JUBA1 | 4\|5\|3 | 12 | 4 | 4\|4\|3 | 0 | 0 | 0 | 0 | 0 | −11.36 | 1.09 | 0.88 |

1. A method of inhibiting inflammatory cell death or hypoxia-induced cell death in a subject in need thereof, comprising
(i) inhibiting the RZ domain of RNF213 protein in the cells of the subject;
(ii) inhibiting a functional ATPase domain of RNF213 protein in the cells of the subject;
(iii) inhibiting RNF213 protein expression or degrading RNF213 protein in the cells of the subject;
(iv) activating the RING domain of RNF213 protein in the cells of the subject;
(v) inhibiting oligomerization of RNF213 protein in the cells of the subject; or
(vi) inhibiting an RNF213 activator in the cells of the subject.

2-6. (canceled)

7. The method of claim 1, wherein the inflammatory cell death is pyroptotic cell death.

8-13. (canceled)

14. A method of preventing or treating a vascular occlusive event or disorder in a subject in need thereof, comprising
(i) inhibiting the RZ domain of RNF213 protein in the cells of the subject;
(ii) inhibiting a functional ATPase domain of RNF213 protein in the cells of the subject;
(iii) inhibiting RNF213 protein expression or degrading RNF213 protein in the cells of the subject;
(iv) activating the RING domain of RNF213 protein in the cells of the subject;
(v) inhibiting oligomerization of RNF213 protein in the cells of the subject; or
(vi) inhibiting an RNF213 activator in the cells of the subject.

15-19. (canceled)

20. The method of claim 14 wherein the vascular occlusive event or disorder is a stroke, myocardial infarction, arterial thrombosis, atherosclerosis, peripheral arterial disease, renal vascular disease, Moyamoya disease (MMD), or Moyamoya syndrome.

21. A method of preventing or treating an autoimmune or inflammatory disease in a subject in need thereof, comprising
(i)inhibiting the RZ domain of RNF213 protein in the cells of the subject;
(ii) inhibiting a functional ATPase domain of RNF213 protein in the cells of the subject;
(iii) inhibiting RNF213 protein expression or degrading RNF213 protein in the cells of the subject;
(iv) activating the RING domain of RNF213 protein in the cells of the subject;
(v) inhibiting oligomerization of RNF213 protein in the cells of the subject; or
(vi) inhibiting an RNF213 activator in the cells of the subject.

22-26. (canceled)

27. The method of claim 21, wherein the autoimmune or inflammatory disease is Graves' disease, systemic lupus erythematosus, Sjögren's syndrome, multiple sclerosis, or rheumatoid arthritis.

28. A method of preventing or treating a disease associated with lipotoxicity in a subject in need thereof, comprising
(i) inhibiting the RZ domain of RNF213 protein in the cells of the subject;
(ii) inhibiting a functional ATPase domain of RNF213 protein in the cells of the subject;
(iii) inhibiting RNF213 protein expression or degrading RNF213 protein in the cells of the subject;
(iv) activating the RING domain of RNF213 protein in the cells of the subject;
(v) inhibiting oligomerization of RNF213 protein in the cells of the subject; or
(vi) inhibiting an RNF213 activator in the cells of the subject.

29-33. (canceled)

34. The method of claim 28, wherein the disease associated with lipotoxicity is hepatic steatosis, non-alcoholic steatohepatitis (NASH), non-alcoholic fatty liver disease (NAFLD), primary lipodystrophy, or secondary lipodystrophy.

35-55. (canceled)

56. The method of claim 1, wherein the RNF213 activator is ABL1/2 or TNK1.

57. The method of claim 56, wherein inhibiting ABL1/2 comprises administering to the subject an effective amount of asciminib, dasatinib, imatinib, AP 24149, ponatinib, ruserontinib, PD173952, bosutinib, rebastinib, olverembatinib, KW-2449, bafetinib, or nilotinib.

58. The method of claim 56, wherein inhibiting TNK1 comprises administering to the subject an effective amount of TP-5801, TP-5801 TFA, XMD8-92, CZC-25146, or CZC-25146 hydrochloride.

59. The method of claim 1, wherein degrading RNF213 protein comprises administering to the subject an effective amount of PROTAC or a molecular glue.

60. The method claim 1, wherein inhibiting RNF213 protein expression comprises administering to the subject an effective amount of an siRNA, an shRNA, an anti-sense oligonucleotide, or a site-specific nuclease.

61-84. (canceled)

85. The method of claim 1, wherein the functional ATPase domain of RNF213 protein is the 3^{rd} or 4^{th} ATPase domain.

86. (canceled)

87. The method of claim 1, wherein the subject is human.
